(19) United States
(12) Patent Application Publication
(10) Pub. No.: US 2025/0179061 A1
(43) Pub. Date: Jun. 5, 2025

(54) RIP1 MODULATORS, PREPARATIONS, AND USES THEREOF

(71) Applicant: SIRONAX LTD., Grand Cayman (KY)

(72) Inventors: Yaning SU, Beijing (CN); Hanying RUAN, Beijing (CN); Yanping XU, Beijing (CN); Yimin JIANG, Beijing (CN); Zhen SUN, Shanghai (CN); Junliang HAO, Shanghai (CN); Bo LI, Beijing (CN); Zhiyuan ZHANG, Beijing (CN)

(21) Appl. No.: 18/840,084

(22) PCT Filed: Feb. 24, 2023
(86) PCT No.: PCT/CN2023/078015

§ 371 Date: Aug. 21, 2024

(30) Foreign Application Priority Data
Feb. 28, 2022 (WO) PCT/CN2022/078424

(51) Int. Cl. C07D 417/12; A61K 31/4196; A61K 31/427; A61K 31/428; A61K 31/4439; A61K 31/496; A61K 31/501; A61K 31/506; A61K 31/5377; A61K 31/538; A61K 31/5386; A61K 31/635; C07D 249/12; C07D 401/10; C07D 401/12; C07D 401/14; C07D 403/10; C07D 403/12; C07D 403/14; C07D 405/10; C07D 405/14; C07D 409/14; C07D 413/12; C07D 413/14; C07D 417/04; C07D 417/14; C07D 471/10; C07D 487/04; C07D 487/10; C07D 491/048; C07D 491/107; C07D 498/04; C07D 498/10

(52) U.S. Cl. CPC C07D 417/12; A61K 31/4196; A61K 31/427; A61K 31/428; A61K 31/4439; A61K 31/496; A61K 31/501; A61K 31/506; A61K 31/5377; A61K 31/538; A61K 31/5386; A61K 31/635; C07D 249/12; C07D 401/10; C07D 401/12; C07D 401/14; C07D 403/10; C07D 403/12; C07D 403/14; C07D 405/10; C07D 405/14; C07D 409/14; C07D 413/12; C07D 413/14; C07D 417/04; C07D 417/14; C07D 471/10; C07D 487/04; C07D 487/10; C07D 491/048; C07D 491/107; C07D 498/04; C07D 498/10

(57) ABSTRACT

This disclosure provides compounds of Formula I, compositions comprising the same, and methods of using the same, including use in treating various diseases and conditions, e.g., including those mediated by receptor-interacting protein 1 (RIP1) signaling.

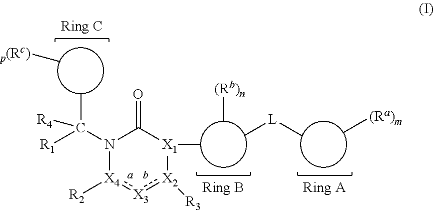

### CROSS REFERENCE TO RELATED APPLICATION

This application claims priority to International Application No. PCT/CN2022/078424, filed on Feb. 28, 2022, the content of which is incorporated by reference in its entirety.

### FIELD OF THE DISCLOSURE

The present disclosure relates to compounds that modulate the receptor-interacting protein 1 (RIP1), compositions comprising the compounds, methods of preparing the compounds, and methods of using the compounds to treat various diseases or conditions, e.g., those mediated by RIP1.

### BACKGROUND OF THE DISCLOSURE

Necroptosis, an important form of programmed cell death (PCD), is a highly regulated caspase-independent type of cell death that plays a critical role in many necrotic cell diseases, manifested in various pathological forms of cell death, including ischemic brain injury, neurodegenerative diseases, viral infections, and peripheral autoimmune diseases. (Dunai, et al., December 2011, Pathol. Oncol. Res.: POR 17 (4): 791-800. J. Med. Chem. 2020, 63, 4, 1490-1510. Nature Reviews Drug Discovery, 19, 553-571(2020)). Tumor necrosis factor alpha (TNF-α)-induced NF-κB activation plays a central role in the immune system and inflammatory responses.

Receptor-interacting protein 1 (RIP1) is a multi-functional signal transducer involved in mediating nuclear factor κB (NF-κB) activation, apoptosis, and necroptosis. The kinase activity of RIP1 is critically involved in mediating necroptosis, a caspase-independent pathway of necrotic cell death. (Holler et al. Nat Immunol 2000; 1: 489-495; Degterev et al. Nat Chem Biol 2008; 4: 313-321). RIP1 can contribute to PD-1 immunotherapy resistance (e.g., Manguso et al., 2017 Nature 547, 413-418) and can act as a checkpoint kinase governing tumor immunity (e.g., Wang et al, Cancer Cell 34, 757-774, Nov. 12, 2018). RIP1 has emerged as a promising therapeutic target for the treatment of a wide range of human neurodegenerative, autoimmune, and inflammatory diseases, such as psoriasis, rheumatoid arthritis, and ulcerative colitis (Pharmacol. Res. Perspect. 2017, 5, e00365, PNAS May 14, 2019 116 (20) 9714-9722), as well for CNS indications such as ALS and Alzheimer's disease. (Nat. Rev. Neurosci. 2019, 20, 19-33).

Certain compounds for modulating necrosis or necroptosis are disclosed in U.S. Pat. Nos. 9,974,762, 10,092,529, 6,756,394, 8,278,344, U.S. Patent Publication No. 20120122889, U.S. Patent Publication No. 20090099242, U.S. Patent Publication No. 20100317701, U.S. Patent Publication No. 20110144169, U.S. Patent Publication No. 20030083386, U.S. Patent Publication No. 201200309795, WO2009023272, WO2010075290, WO2010075561, WO2012125544, WO 2020/103884, WO-2021233397, WO-2021233396, WO-2021233394, WO-2020103884, and WO-2020103859.

### SUMMARY OF THE DISCLOSURE

One aspect of this disclosure provides a compound selected from compounds of the Formulae disclosed herein (e.g., Formulae I, IIa, IIb, IIc, IId, IIIa, IIIb, IIIc, IIId, IIIe, IIIf, IVa, IVb, Va, Vb, Vc, Vd, VIa, VIb, VIc, VId, VIe, VIf, VIIa, VIIb, VIIc, VIIIa, VIIIb, VIIIc, VIIId, VIIIe, VIIIf, and Compounds 1 to 702) disclosed herein, a tautomer thereof, a solvate or stereoisomer of the compound or the tautomer, or a pharmaceutically acceptable salt of the foregoing, which can be employed in the treatment of various diseases or conditions, such as diseases or conditions caused by axonal degeneration. For example, disclosed herein is a compound of the following structural Formula I:

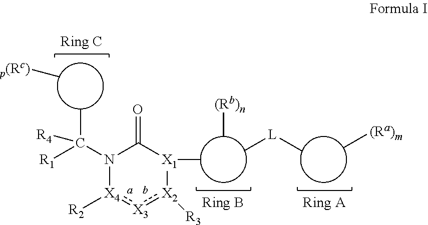

a tautomer thereof, a solvate or stereoisomer of the compound or the tautomer, or a pharmaceutically acceptable salt of the foregoing.

In one aspect of the disclosure, the compounds of the Formulae disclosed herein are selected from Compounds 1 to 702 shown below, a tautomer thereof, a solvate or stereoisomer of the compound or the tautomer, or a pharmaceutically acceptable salt of the foregoing.

In some embodiments, the disclosure provides pharmaceutical compositions comprising a compound of the Formulae disclosed herein, a tautomer thereof, a hydrate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 702 shown below, a tautomer thereof, a hydrate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of the Formulae disclosed herein, a tautomer thereof, a hydrate or stereoisomer of the compound or the tautomer, or a pharmaceutically acceptable salt of the foregoing, or a pharmaceutical composition comprising any of the foregoing, wherein the disease or condition is selected from an inflammatory disease, an immune disease (e.g., an autoimmune disease), an allergic disease, transplant rejection, a necrotic cell disease, a neurodegenerative disease, a central nervous system (CNS) disease, an ocular disease, an infectious disease, and a malignancy.

A further aspect of the disclosure provides methods of treating a disease or condition mediated by RIP1, comprising administering to a subject in need thereof, a therapeutically effective amount of a compound of the Formulae disclosed herein, a tautomer thereof, a hydrate or stereoisomer of the compound or the tautomer, or a pharmaceutically acceptable salt of the foregoing, or a pharmaceutical composition comprising any of the foregoing.

In some embodiments, the methods of treatment comprise administering to a subject in need thereof, a compound selected from Compounds 1 to 702 shown below, a tautomer thereof, a hydrate or stereoisomer of the compound or the tautomer, or a pharmaceutically acceptable salt of the foregoing, or a pharmaceutical composition comprising any of the foregoing.

In some embodiments, the methods of treatment comprise administration of an additional active pharmaceutical agent to the subject in need thereof, either in the same pharmaceutical composition as a compound of the Formulae disclosed herein, a tautomer thereof, a hydrate or stereoisomer of the compound or the tautomer, or a pharmaceutically acceptable salt of the foregoing, or in a separate composition. In some embodiments, the methods of treatment comprise administering a compound selected from Compounds 1 to 702 shown below, a tautomer thereof, a hydrate or stereoisomer of the compound or the tautomer, or a pharmaceutically acceptable salt of the foregoing with an additional active pharmaceutical agent either in the same pharmaceutical composition or in a separate composition. When administered as a separate dosage form, the additional therapeutic agent may be administered prior to, at the same time as, or following administration of the compound, tautomer, hydrate, stereoisomer, or a pharmaceutically acceptable salt disclosed herein.

Also disclosed herein are methods of mediating, e.g., inhibiting, RIP1, comprising contacting the RIP1 protein or a fragment thereof with a compound of the Formulae disclosed herein, a tautomer thereof, a hydrate or stereoisomer of the compound or the tautomer, or a pharmaceutically acceptable salt of the foregoing, or a pharmaceutical composition comprising any of the foregoing. In some embodiments, the methods of inhibiting RIP1 comprise contacting the RIP1 protein or a fragment thereof with a compound selected from Compounds 1 to 702 shown below, a tautomer thereof, a hydrate or stereoisomer of the compound or the tautomer, or a pharmaceutically acceptable salt of the foregoing, or a pharmaceutical composition comprising any of the foregoing.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C↓C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “ring” or “ring system” refers to a monocyclic and a polycyclic (e.g., bicyclic and tricyclic) group. A ring can be a carbon cycle or heterocycle, aromatic or non-aromatic. For example, a bicyclic ring can be a fused, bridged, or spiro cyclic system.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, 3-10, 3-8, 3-6, 3-4, or 5-6 carbon atoms. Even further for example, the cycloalkyl group may be a monocyclic group of 3-12, 3-8, 3-6, 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6], and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not an aromatic ring, as “aromatic ring” is defined herein.

The term “heterocyclic” or “heterocycle” or “heterocyclyl” refers to a ring selected from 3- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from, e.g., oxygen, sulfur, nitrogen, and silicon. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein.

Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1, 1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6], and [6,6] ring systems as mentioned above; a fused bicyclic aryl ring such as 7- to 12-membered bicyclic aryl ring systems as mentioned above, a fused tricyclic aryl ring such as 10- to 15-membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, phosphorus, and silicon, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds. A double bond may be depicted as [symbol] (two solid lines). The depiction of [symbol] (a solid line and a dashed line), as used herein, denotes a bond that may be a double bond or a single bond.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN,” “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2]p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

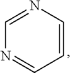

or as a cycle with an inside circle, such as

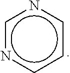

The term “aryl” herein refers to a group selected from: monocyclic carbocyclic aromatic rings, for example, phenyl; bicyclic ring systems such as 7-12 membered, e.g., 9-10 membered, bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O)R′, —C(═O)NR′R″, or —C(═O)OR′, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereoisomers, and conversion to the pure stereoisomers, and (3) separation of the substantially pure or enriched stereoisomers directly under chiral conditions.

In the present disclosure, certain single stereoisomers, e.g., substantially pure enantiomers, are separated from one another, e.g., by a chiral separation. However, the absolute configuration of certain separated single stereoisomers are not presently known. Such unknown chiral centers are denoted with a * in the structural formulae and the corresponding compounds are denoted as “single unknown stereoisomer.” For instance, Compounds 3 and 4 (Examples 3 and 4) were synthesized and separated by chiral separation and the chemical structures of Compounds 3 and 4 each bear a star to indicate the unknown absolute configuration, and indicated as “single unknown stereoisomer.”

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n—COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺ (C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂H or —CDH₂ contains one or more deuteriums in place of hydrogen. For example, the compounds may be radiolabeled with radioactive isotopes, such as for example tritium (³H), iodine-125 (¹²⁵I), or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position.

Combinations of chemical components, e.g., substituents, ring structures, linkers (L), and/or heteroatoms, envisioned by this disclosure are those that result in the formation of stable or chemically feasible compounds.

In some embodiments, substituents are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, ═NR′, ═N—OR′, —NR′R″, —SR′, —SiR′R″R′″, —OC(═O)R′, —C(═O)R′, —CO₂R′, —C(═O)NR′R″, —OC(═O)NR′R″, —NR″C(═O)R′, —NR′—C(═O)NR″R′″, —NR′—SO₂NR″R′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN, —NO₂, —N₃, —CH(Ph)₂, perfluoro(C₁-C₄)alkoxy, and perfluoro(C₁-C₄)alkyl, in a number ranging from zero to three, with those groups having zero, one, or two substituents being particularly preferred. R′, R″, and R′″ each independently refer to hydrogen, unsubstituted C₁-C₈ alkyl and heteroalkyl, C₁-C₈ alkyl and heteroalkyl substituted with one to three halogens, unsubstituted aryl, aryl substituted with one to three halogens, unsubstituted alkyl, alkoxy, or thioalkoxy groups, or aryl-(C₁-C₄) alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, ═O, —NR′R″, —SR′, —SiR′R″R′″, —OC(═O)R′, —C(═O)R′, —CO₂R′, —C(═O)NR′R″, —OC(═O)NR′R″, —NR″C(═O)R′, —NR″CO₂R′, —NR′—SO₂NR″R′″, —S(═O)R′, —SO₂R′, —SO₂NR′R″, —NR″SO₂R, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₆-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, isocyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

In some embodiments, substituents are structurally depicted herein. For example, a ring substituted by R at any chemically feasible position can be depicted as

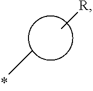

wherein the symbol “*” denotes a point of connection to another component of a molecule. In another example, a bicyclic group substituted by R at any chemically feasible position can be depicted as

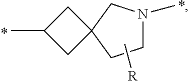

wherein the symbol “*” denotes a point of connection to another component of a molecule and R can be substituted at any chemically feasible position on the 4-membered ring or the 5-membered ring of the bicyclic group.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography.

Chromatography can involve any number of methods including, for example, reverse-phase and normal phase; size exclusion; ion exchange; high, medium, and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., ALS, Parkinson's disease, multiple sclerosis, traumatic brain injury, diabetic neuropathy, and CIPN. The disease or condition may be caused by axonal degeneration. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., ALS, Parkinson's disease, multiple sclerosis, traumatic brain injury, diabetic neuropathy, and CIPN. The disease or condition may be caused by axonal degeneration. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages, for example, a range of ±10% with respect to a specific point value) that is recognized by one of ordinary skill in the art.

### II. Compounds and Compositions

In a first embodiment, a compound of this disclosure is a compound of the following structural Formula I:

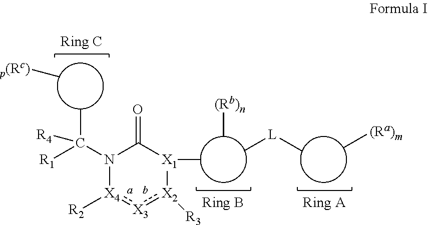

a tautomer thereof, a solvate or stereoisomer of the compound or the tautomer, or a pharmaceutically acceptable salt of the foregoing, wherein:
X₁ is C or N; X₂ is C or N; X₃ is C, N, or absent; X₄ is C or N;
Ring A is phenyl, 5- to 9-membered heteroaryl, 5- to 6-membered cycloalkyl, or 5- to 9-membered heterocyclyl;
Ring B is phenyl, 5- to 9-membered heteroaryl, 5- to 6-membered cycloalkyl, or 4- to 8-membered heterocyclyl;
Ring C is phenyl, 5- to 6-membered heteroaryl, 5- to 6-membered cycloalkyl, or 5- to 6-membered heterocyclyl;
bond a and bond b are each independently selected from a single bond and a double bond, provided that bond a and bond b cannot be double bond at the same time, and when X₃ is absent, the bond between X₂ and X₄ is a single bond or a double bond;
R^{a}, for each occurrence, is independently selected from halogen, cyano, ═O, NO₂, optionally substituted C₁ to C₆ alkyl, optionally substituted C₂ to C₆ alkenyl, optionally substituted C₂ to C₆ alkynyl, optionally substituted acyl, optionally substituted 3 to 10-membered cycloalkyl, optionally substituted 3 to 10-membered heterocyclyl, optionally substituted phenyl, optionally substituted 5 to 10-membered heteroaryl, optionally substituted nitrogen, and optionally substituted oxygen;
R^{b}, for each occurrence, is independently selected from halogen, CN, ═O, and C₁-C₄ alkyl optionally substituted by 1 to 3 groups of halogen;
R^{c}, for each occurrence, is independently, is selected from halogen, CN, C₁-C₆ alkyl, OR^{s1}, and —C(═O)OR^{s1};
R₁ is H, R₂ is selected from H, halogen, CN, OR^{s1}, —NP^{p1}R^{q1}, ═O, and C₁ to C₃ alkyl optionally substituted by 1 to 3 groups selected from halogen, or R₁ and R₂ join to form a 5- to 6-membered carbocycle or heterocycle optionally substituted by 1 to 3 groups selected from halogen and C₁ to C₃ alkyl optionally substituted by 1 to 3 groups selected from halogen;
R₃ is selected from H and ═O, provided that when R₃ is ═O, X₂ is C;
R₄ is selected from H and C₁ to C₃ alkyl;
L is selected from —NR^{x}—, —(CH₂)ᵤO(CH₂)ᵤ—, —(CH₂)ᵤS(═O)_{w}—(CH₂)ᵤ—, —S(═O)_{w}(═NR^{x})—, —NR^{x}S(═O)_{w}—, —S(═O)_{w}(NR^{x})—, —C(═O)—, and C₁-C₃ alkylene, wherein the C₁-C₃ alkylene of L is optionally substituted by 1 to 2 groups selected from OH, C₁-C₃ alkyl, and ═CHR^{x}, wherein the C₁-C₃ alkyl of the C₁-C₃ alkylene of L optionally join to form a C₃-C₄ cycloalkyl; wherein
R^{p1} and R^{q1}, for each occurrence, are each independently selected from hydrogen and C₁-C₄ alkyl optionally substituted with 1 to 3 groups selected from halogen, CN, and OH;
R^{s1}, for each occurrence, is independently selected from hydrogen and C₁-C₄ alkyl optionally substituted with 1 to 3 groups selected from halogen, CN, and OH; and
R^{x} is selected from H and C₁-C₄ alkyl;
m and p are each an integer independently selected from 0, 1, 2, 3, and 4;
n is an integer selected from 0, 1, and 2;
w, for each occurrence, is an integer independently selected from 0, 1, and 2;
u, for each occurrence, is an integer independently selected from 0, 1, and 2; provided that the compounds is not:

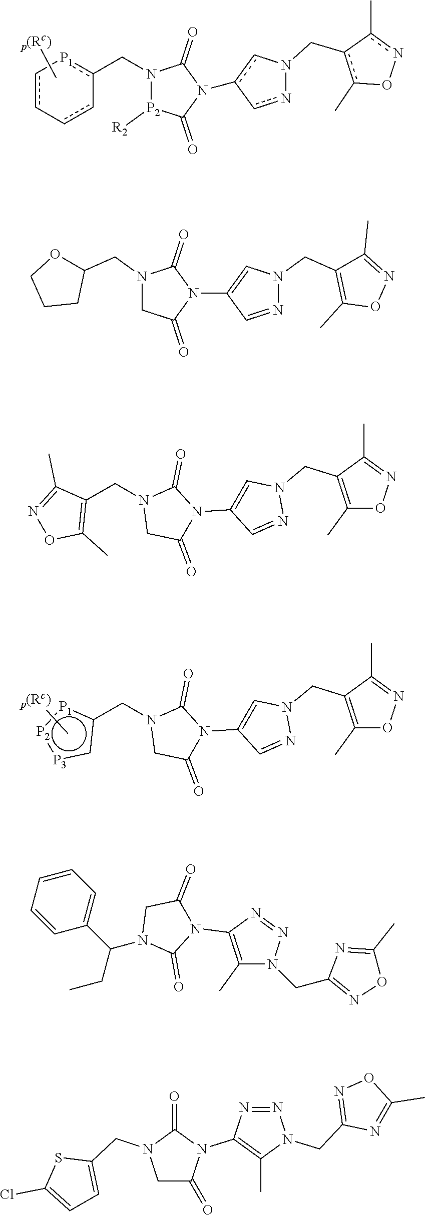

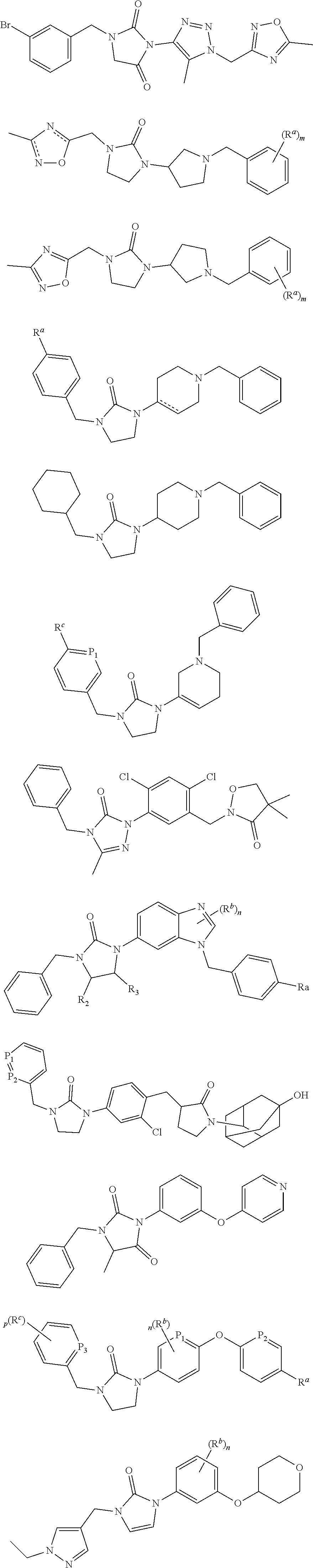

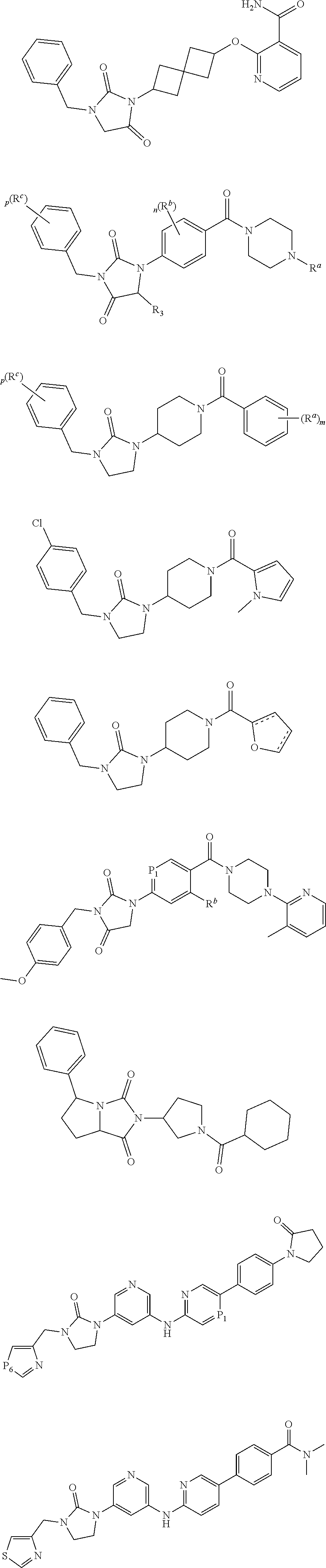

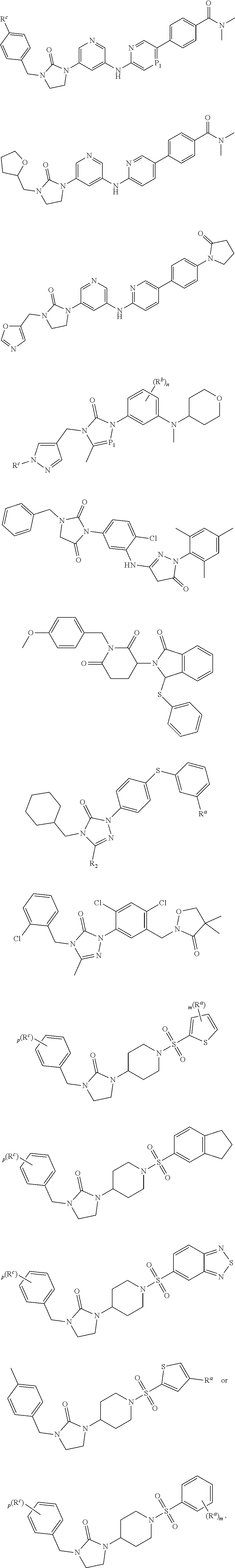

wherein P₁, P₂, and P₃, for each occurrence, are each independently selected from C and N, and P₆ is independently selected from S and O.

Combinations of substituents or other variations (e.g., optional presence of heteroatoms in a ring) as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In a second embodiment, in a compound, tautomer, a solvate or stereoisomer of the compound or the tautomer, or pharmaceutically acceptable salt of this disclosure, Ring A is a phenyl, pyridinyl, pyrimidinyl, pyrazinyl, thiazolyl, pyrazolyl, imidazolyl, pyrrolyl, pyridazinyl, piperazinyl, oxazolyl, isoxazolyl, triazolyl, cyclopentyl, cyclohexanyl, tetrahydro-furanyl, or tetrahydro-pyranyl group; and all other variables not specifically defined herein are defined in the preceding embodiment.

In a third embodiment, in a compound, tautomer, a solvate or stereoisomer of the compound or the tautomer, or pharmaceutically acceptable salt of this disclosure, Ring B is a phenyl, pyridinyl, thiazolyl, cyclopentenyl, cyclobutanyl, cyclohexanyl, piperidyl, or pyrrolidinyl group, or a 5- to 8-membered bicyclic group optionally containing one or two N atoms; and all other variables not specifically defined herein are defined in any one of the preceding embodiments.

In a fourth embodiment, in a compound, tautomer, a solvate or stereoisomer of the compound or the tautomer, or pharmaceutically acceptable salt of this disclosure, Ring C is phenyl, pyridinyl, thiazolyl, isothiazolyl, oxazolyl, cyclopentyl, cyclopentenyl, cyclohexanyl, cyclohexenyl, isoxazolyl, tetrahydro-pyranyl, or dihydro-pyranyl group; and all other variables not specifically defined herein are defined in any one of the preceding embodiments.

In a fifth embodiment, a compound of the disclosure is one of the following structural formula IIa:

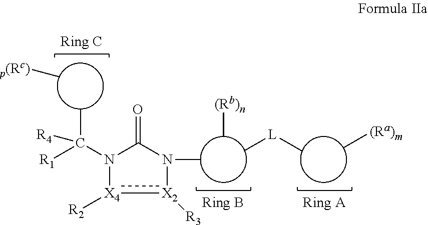

a tautomer thereof, a solvate or stereoisomer of the compound or the tautomer, or a pharmaceutically acceptable salt of the foregoing, wherein R¹ and R² do not join to form a 5- to 6-membered carbocycle or heterocycle; and all other variables not specifically defined herein are as defined in any one of the preceding embodiments.

In a sixth embodiment, a compound of the disclosure is one of the following structural formula IIb:

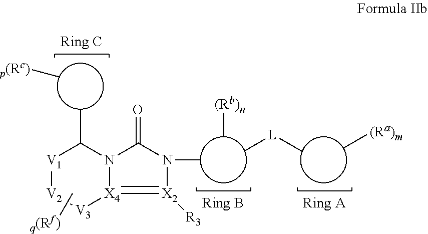

a tautomer thereof, a solvate or stereoisomer of the compound or the tautomer, or a pharmaceutically acceptable salt of the foregoing, wherein V₁, V₂, and V₃ are each independently selected from C, O, and N, R^{f}, for each occurrence, is independently selected from C₁-C₃ alkyl and halogen, and q is selected from 0, 1, and 2; and all other variables not specifically defined herein are as defined in any of the appropriate preceding embodiments.

In a seventh embodiment, a compound of the disclosure is one of the following structural formula IIc:

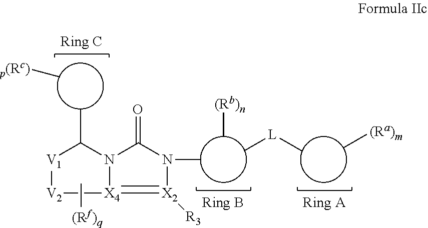

a tautomer thereof, a solvate or stereoisomer of the compound or the tautomer, or a pharmaceutically acceptable salt of the foregoing, wherein V₁ and V₂ are each independently selected from C, O, and N, R^{f}, for each occurrence, is independently selected from C₁-C₃ alkyl and halogen, and q is selected from 0, 1, and 2; and all other variables not specifically defined herein are as defined in any of the appropriate preceding embodiments.

In an eighth embodiment, a compound of the disclosure is one of the following structural formula IId:

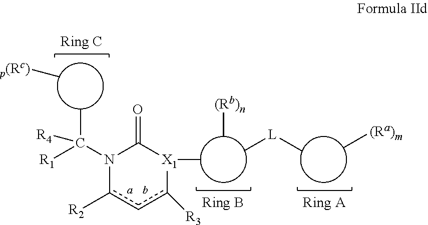

a tautomer thereof, a solvate or stereoisomer of the compound or the tautomer, or a pharmaceutically acceptable salt of the foregoing; wherein R¹ and R² do not join to form a 5- to 6-membered carbocycle or heterocycle; and all other variables not specifically defined herein are as defined in any of the appropriate preceding embodiments.

In a ninth embodiment, a compound of the disclosure is one of the following structural formula IIIa:

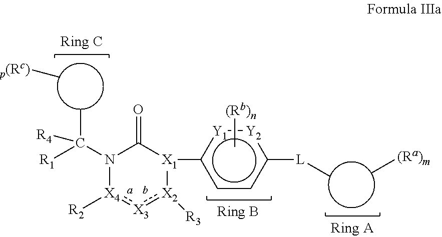

a tautomer thereof, a solvate or stereoisomer of the compound or the tautomer, or a pharmaceutically acceptable salt of the foregoing; wherein Y₁ and Y₂ are each independently selected from C and N; and all other variables not specifically defined herein are as defined in any of the appropriate preceding embodiments.

In a tenth embodiment, a compound of the disclosure is one of the following structural formula IIIb:

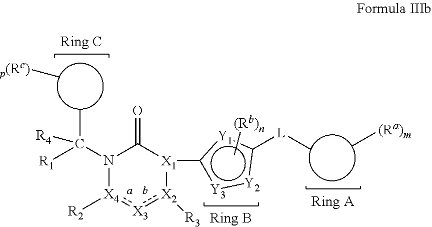

a tautomer thereof, a solvate or stereoisomer of the compound or the tautomer, or a pharmaceutically acceptable salt of the foregoing; wherein Y₁, is selected from S, C, O, and N, Y₂ and Y₃ are each independently selected from S, C, O and N; and all other variables not specifically defined herein are as defined in any of the appropriate preceding embodiments.

As disclosed herein, combinations of heteroatoms are those that result in the formation of stable or chemically feasible compounds. For example, with respect to Formula IIIb, the clause “Y₂ and Y₃ are each independently selected from S, C, O and N” refer to those combinations of heteroatoms that result in the formation of stable or chemically feasible compounds. For instance, when Y₂ and Y₃ of Formula IIIb are both O, such combination of Y₂ and Y₃ would result in unstable or chemically infeasible compounds. Such unstable or chemically infeasible compounds are not intended to be covered by the compounds of this disclosure.

In an eleventh embodiment, a compound of the disclosure is one of the following structural formula IIIc:

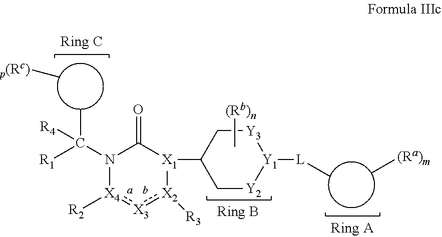

a tautomer thereof, a solvate or stereoisomer of the compound or the tautomer, or a pharmaceutically acceptable salt of the foregoing, Y₁ is selected from C and N, Y₂ and Y₃ are each independently C or absent; and all other variables not specifically defined herein are as defined in any of the appropriate preceding embodiments.

In a twelfth embodiment, a compound of the disclosure is one of the following structural formula IIId:

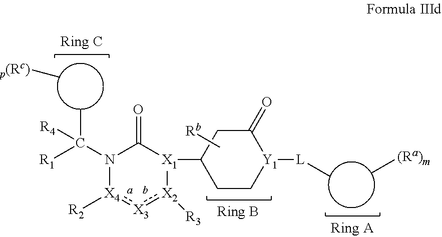

a tautomer thereof, a solvate or stereoisomer of the compound or the tautomer, or a pharmaceutically acceptable salt of the foregoing, Y₁ is selected from C and N; and all other variables not specifically defined herein are as defined in any of the appropriate preceding embodiments.

In a thirteenth embodiment, a compound of the disclosure is one of the following structural formula IIIe:

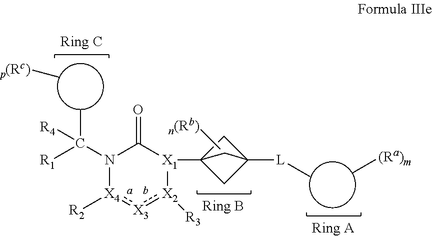

a tautomer thereof, a solvate or stereoisomer of the compound or the tautomer, or a pharmaceutically acceptable salt of the foregoing; and all other variables not specifically defined herein are as defined in any of the appropriate preceding embodiments.

In a fourteenth embodiment, a compound of the disclosure is one of the following tructural formula IIIf

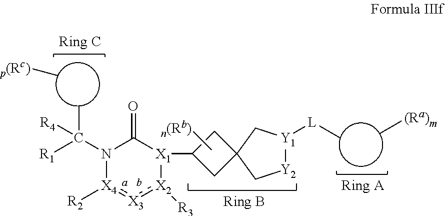

a tautomer thereof, a solvate or stereoisomer of the compound or the tautomer, or a pharmaceutically acceptable salt of the foregoing, wherein Y₁ is selected from C and N; and all other variables not specifically defined herein are as defined in any of the appropriate preceding embodiments.

In a fifteenth embodiment, a compound of the disclosure is one of the following structural formula IVa:

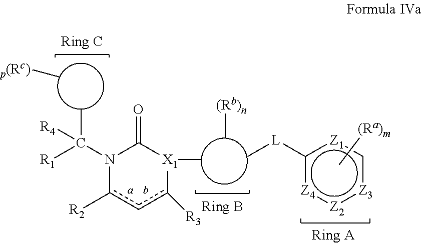

a tautomer thereof, a solvate or stereoisomer of the compound or the tautomer, or a pharmaceutically acceptable salt of the foregoing, wherein Z₁, Z₂, Z₃ and Z₄ are each independently selected from C and N; and all other variables not specifically defined herein are as defined in any of the appropriate preceding embodiments.

In a sixteenth embodiment, a compound of the disclosure is one of the following structural formula IVb:

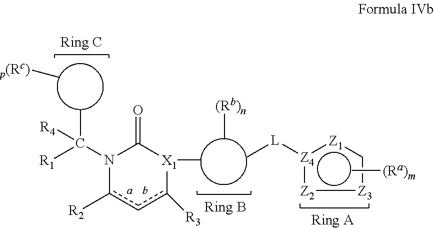

a tautomer thereof, a solvate or stereoisomer of the compound or the tautomer, or a pharmaceutically acceptable salt of the foregoing, wherein Z₁, Z₂, and Z₃, are each independently selected from S, O, C, and N, and Z₄ is selected from C and N; and all other variables not specifically defined herein are as defined in any of the appropriate preceding embodiments.

In a seventeenth embodiment, a compound of the disclosure is one of the following structural formula Va:

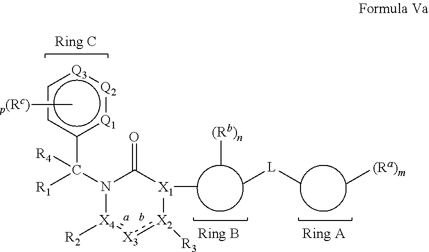

a tautomer thereof, a solvate or stereoisomer of the compound or the tautomer, or a pharmaceutically acceptable salt of the foregoing, wherein Q₁, Q₂, and Q₃ are each independently selected from C and N; and all other variables not specifically defined herein are as defined in any of the appropriate preceding embodiments.

In an eighteenth embodiment, a compound of the disclosure is one of the following structural formula Vb:

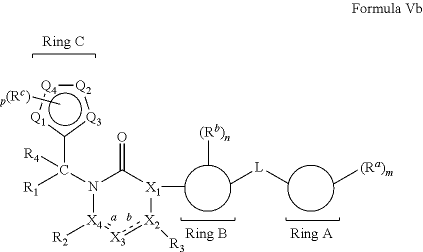

a tautomer thereof, a solvate or stereoisomer of the compound or the tautomer, or a pharmaceutically acceptable salt of the foregoing, wherein Q₁, Q₂, Q₃, and Q₄ are each independently selected from C, N, S, and O; and all other variables not specifically defined herein are as defined in any of the appropriate preceding embodiments.

In a nineteenth embodiment, a compound of the disclosure is one of the following structural formula Vc:

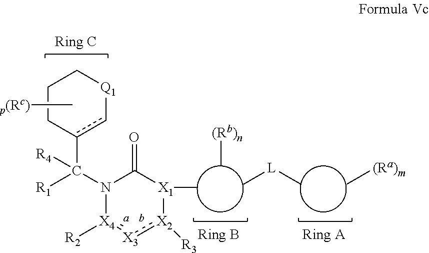

a tautomer thereof, a solvate or stereoisomer of the compound or the tautomer, or a pharmaceutically acceptable salt of the foregoing, wherein Q₁ is C, O, or absent; and all other variables not specifically defined herein are as defined in any of the appropriate preceding embodiments.

In a twentieth embodiment, a compound of the disclosure is one of the following structural formula Vd:

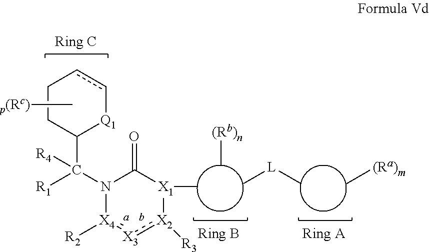

a tautomer thereof, a solvate or stereoisomer of the compound or the tautomer, or a pharmaceutically acceptable salt of the foregoing, wherein Q₁ is C or absent; and all other variables not specifically defined herein are as defined in any of the appropriate preceding embodiments.

In a twenty-first embodiment, a compound of the disclosure is one of the following structural formula VIa:

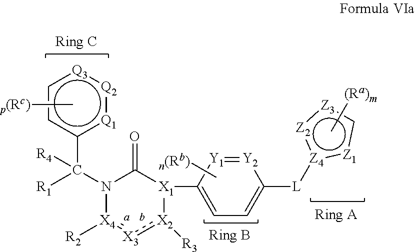

a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z₁, Z₂, and Z₃, are each independently selected from S, O, C, and N, Z₄ is selected from C and N; and all other variables not specifically defined herein are as defined in any of the appropriate preceding embodiments.

In a twenty-second embodiment, a compound of the disclosure is one of the following structural formula VIb:

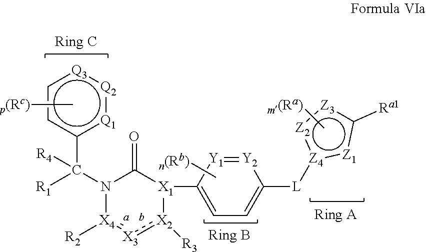

a tautomer thereof, a solvate or stereoisomer of the compound or the tautomer, or a pharmaceutically acceptable salt of the foregoing, wherein R^{a}, for each occurrence, is independently selected from absent, halogen, CN, NO₂, NH₂, —NH(C₁-C₃ alkyl), —OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{a} is selected from optionally substituted C₁-C₃ alkyl, optionally substituted 3- to 6-membered cycloalkyl, optionally substituted 3- to 6-membered heterocyclyl, —NC(═O)R^{p2}, and —NR^{p2}R^{q2}; wherein R^{p2} and R^{q2}, for each occurrence, are each independently selected from hydrogen and optionally substituted C₁-C₆ alkyl, or R^{p2} and R^{q2} join and form an optionally substituted 3 to 10-membered heterocyclyl; and all other variables not specifically defined herein are as defined in any of the appropriate preceding embodiments.

In a twenty-third embodiment, in a compound, tautomer, a solvate or stereoisomer of the compound or the tautomer, or pharmaceutically acceptable salt of this disclosure, R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, and NO₂, and R^{a1} is selected from COOMe, COOEt,

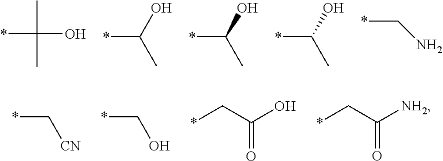

and —NR^{p2}R^{q2}, wherein R^{p2} and R^{q2} join and form a 3 to 10-membered heterocyclyl optionally substituted by 1 to 2 groups selected from halogen, OH, CN, —O(C₁-C₃ alkyl), NH₂, NHBoc, NH(C₁-C₃ alkyl), and C₁-C₃ alkyl optionally substituted with 1 to 3 groups selected from halogen, CN, NH₂, NHBoc, and OH; and all other variables not specifically defined herein are as defined in any of the appropriate preceding embodiments.

In a twenty-fourth embodiment, a compound of the disclosure is one of the following structural formula VIc:

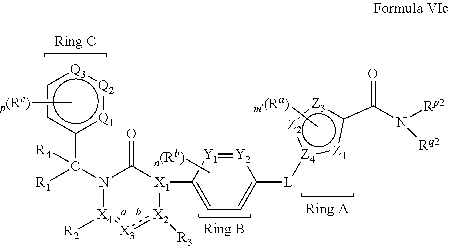

a tautomer thereof, a solvate or stereoisomer of the compound or the tautomer, or a pharmaceutically acceptable salt of the foregoing, wherein R^{a}, for each occurrence, is independently selected from absent, halogen, CN, NO₂, NH₂, —NH(C₁-C₃ alkyl), OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{p2} and R^{q2} are independently selected from H, optionally substituted C₁ to C₆ alkyl, optionally substituted 3 to 10-membered cycloalkyl, optionally substituted 3 to 10-membered heterocyclyl, and optionally substituted 3 to 10-membered heteroaryl; and all other variables not specifically defined herein are as defined in any of the appropriate preceding embodiments.

In a twenty-fifth embodiment, in a compound, tautomer, a solvate or stereoisomer of the compound or the tautomer, or pharmaceutically acceptable salt of this disclosure, R^{a}, for each occurrence, is independently selected from absent, CH₃, CF₂, F, Cl, CN, NH₂, NHCH₃, and OH; and all other variables not specifically defined herein are as defined in any of the appropriate preceding embodiments.

In a twenty-sixth embodiment, a compound of the disclosure is one of the following structural formula VId:

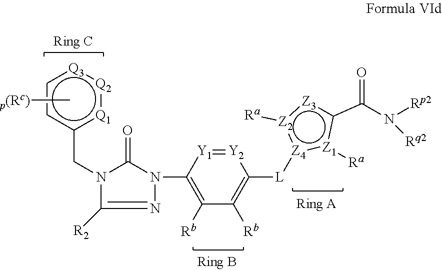

a tautomer thereof, a solvate or stereoisomer of the compound or the tautomer, or a pharmaceutically acceptable salt of the foregoing, wherein L is selected from —O—, —N(R^{x})—, —CH₂—, —S—, —S(═O)—, —S(═O)₂—, and C₁-C₃ alkylene optionally substituted by 1 to 2 groups selected from C₁-C₂ alkyl, wherein the C₁-C₂ alkyl of the C₁-C₃ alkylene of L optionally join to form a C₃-C₄ cycloalkyl, R^{a}, for each occurrence, is independently selected from H and C₁-C₃ alkyl (e.g., C₃ alkyl, C₂ alkyl, preferably methyl), R^{b}, for each occurrence, is independently selected from absent, F, Cl, Br, CH₃, and CN, R^{c} is selected from F, Cl, Br, CH₃, —OCH₃, and CN, p is 0, 1, or 2, provided that when L is —O— or —N(R^{x})—, Z₄ is C, and when L is —CH₂—, —S—, —S(═O)—, —S(═O)₂—, or C₁-C₃ alkylene, Z₄ is C or N; and all other variables not specifically defined herein are as defined in any of the appropriate preceding embodiments.

In a twenty-seventh embodiment, a compound of the disclosure is one of the following structural formula VIe:

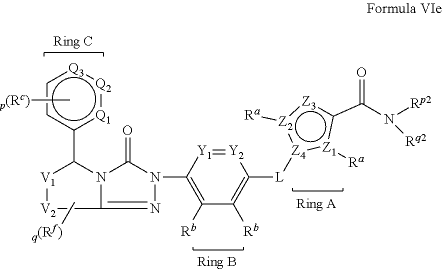

a tautomer thereof, a solvate or stereoisomer of the compound or the tautomer, or a pharmaceutically acceptable salt of the foregoing, wherein V₁ and V₂ are each independently selected from C, N, and O, L is selected from —O—, —N(R^{x})—, —CH₂—, —S—, —S(═O)—, —S(═O)₂—, and C₁-C₃ alkylene optionally substituted by 1 to 2 groups selected from C₁-C₂ alkyl, wherein the C₁-C₂ alkyl of the C₁-C₃ alkylene of L optionally join to form a C₃-C₄ cycloalkyl, R^{a}, for each occurrence, is independently selected from H and C₁-C₃ alkyl (e.g., C₃ alkyl, C₂ alkyl, preferably methyl), R^{b}, for each occurrence, is independently selected from absent, F, Cl, Br, CH₃, and CN, R^{c} is selected from F, Cl, Br, —OCH₃, CH₃, and CN, R^{f}, for each occurrence, is independently selected from C₁-C₃ alkyl and halogen, p is 0, 1, or 2, q is 0, 1, or 2, provided that when L is —O— or —N(R^{x})—, Z₄ is C, and when L is —CH₂—, —S—, —S(═O)—, —S(═O)₂—, or C₁-C₃ alkylene, Z₄ is C or N; and all other variables not specifically defined herein are as defined in any of the appropriate preceding embodiments.

In a twenty-eighth embodiment, a compound of the disclosure is one of the following structural formula VIf

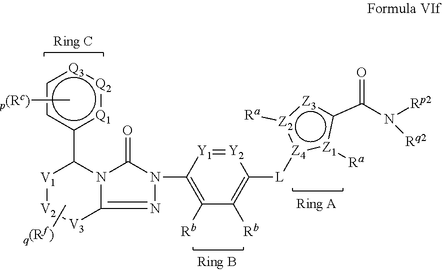

a tautomer thereof, a solvate or stereoisomer of the compound or the tautomer, or a pharmaceutically acceptable salt of the foregoing, wherein V₁, V₂, and V₃ are each independently selected from C, N, and O, L is selected from —O—, —N(R^{x})—, —CH₂—, —S—, —S(═O)—, —S(═O)₂—, and C₁-C₃ alkylene optionally substituted by 1 to 2 groups selected from C₁-C₂ alkyl, wherein the C₁-C₂ alkyl of the C₁-C₃ alkylene of L optionally join to form a C₃-C₄ cycloalkyl, R^{a}, for each occurrence, is independently selected from H and C₁-C₃ alkyl (e.g., C₃ alkyl, C₂ alkyl, preferably methyl), R^{b}, for each occurrence, is independently selected from absent, F, Cl, Br, CH₃, and CN, R^{c} is selected from F, Cl, Br, CH₃, —OCH₃, and CN, R^{f}, for each occurrence, is independently selected from C₁-C₃ alkyl and halogen, p is 0, 1, or 2, q is 0, 1, or 2, provided that when L is —O— or —N(R^{x})—, Z₄ is C, and when L is —CH₂—, —S(═O)—, —S(═O)₂—, or C₁-C₃ alkylene optionally substituted by C₃-C₄ cycloalkyl, Z₄ is C or N; and all other variables not specifically defined herein are as defined in any of the appropriate preceding embodiments.

In a twenty-ninth embodiment, a compound of the disclosure is one of the following structural formula VIIa:

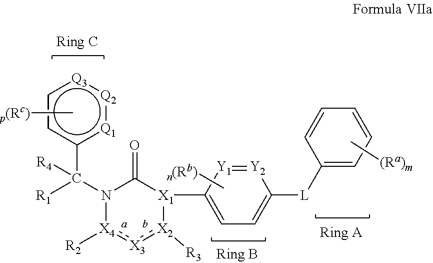

a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N; and all other variables not specifically defined herein are as defined in any of the appropriate receding embodiments.

In a thirtieth embodiment, a compound of the disclosure is one of the following structural formula VIIb:

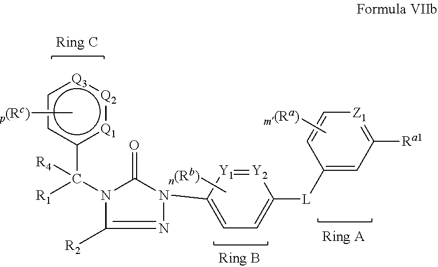

a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z₁ is selected from C and N, R^{a}, for each occurrence, is independently selected from absent, halogen, CN, NO₂, NH₂, —NH(C₁-C₃ alkyl), —OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{a1} is selected from optionally substituted C₁-C₃ alkyl, optionally substituted 3- to 6-membered cycloalkyl, optionally substituted 3- to 6-membered heterocyclyl, —NC(═O)R^{p2}, and —NR^{p2}R^{q2}, wherein R^{p2} and R^{q2}, for each occurrence, are each independently selected from hydrogen and optionally substituted C₁-C₆ alkyl, or R^{p2} and R^{q2} join and form an optionally substituted 3 to 10-membered heterocyclyl; and all other variables not specifically defined herein are as defined in any of the appropriate preceding embodiments.

In a thirty-first embodiment, in a compound, tautomer, a solvate or stereoisomer of the compound or the tautomer, or pharmaceutically acceptable salt of this disclosure, R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, and NO₂, and R^{a1} is selected from COOMe, COOEt,

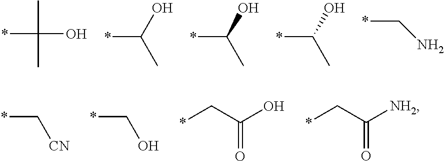

—N^{p2}R^{q2}, wherein R^{p2} and R^{q2} join and form a 3 to 10-membered heterocyclyl optionally substituted by 1 to 2 groups selected from halogen, OH, CN, —O(C₁-C₃ alkyl), NH₂, NHBoc, NH(C₁-C₃ alkyl), and C₁-C₃ alkyl optionally substituted with 1 to 3 groups selected from halogen, CN, NH₂, NHBoc, and OH; and all other variables not specifically defined herein are as defined in any of the appropriate preceding embodiments.

In a thirty-second embodiment, a compound of the disclosure is one of the following structural formula VIIc:

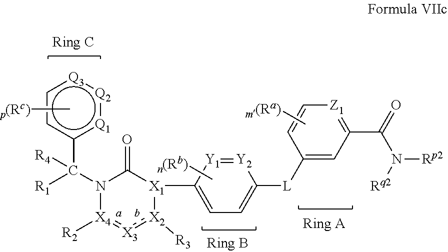

a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z₁ is selected from C and N, R^{a}, for each occurrence is independently selected from absent, halogen, CN, NO₂, NH₂, —NH(C₁-C₃ alkyl), —OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{P2} and R^{q2}, are independently selected from H, optionally substituted C₁ to C₆ alkyl, optionally substituted 3 to 10-membered cycloalkyl, optionally substituted 3 to 10-membered heterocyclyl, and optionally substituted 3 to 10-membered heteroaryl; and all other variables not specifically defined herein are as defined in any of the appropriate preceding embodiments.

In a thirty-third embodiment, in a compound, tautomer, a solvate or stereoisomer of the compound or the tautomer, or pharmaceutically acceptable salt of this disclosure, R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, and NO₂, and wherein the 3 to 10-membered cycloalkyl, 3 to 10-membered heterocyclyl, and 3 to 10-membered heteroaryl of R^{P2} and R^{q2} are optionally substituted with 1 to 2 groups selected from halogen, OH, CN, —O(C₁-C₃ alkyl), NH₂, NHBoc, NH(C₁-C₃ alkyl), and C₁-C₃ alkyl optionally substituted with halogen, CN, NH₂, NHBoc, and OH; and all other variables not specifically defined herein are as defined in any of the appropriate preceding embodiments.

In a thirty-fourth embodiment, a compound of the disclosure is one of the following structural VIIIa, VIIIb, or VIIIc:

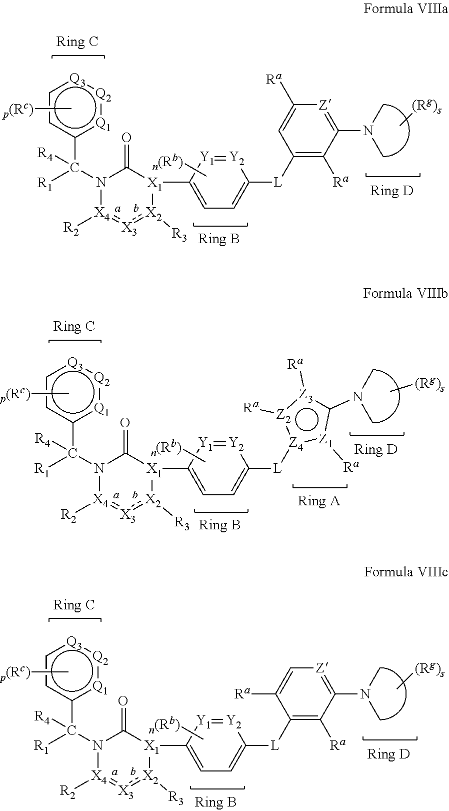

a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z′ is selected from C and N, Z₁, Z₂, and Z₃, are each independently selected from S, O, C, and N, Z₄ is selected from C and N, R^{a}, for each occurrence is independently selected from absent, halogen, CN, NO₂, NH₂, —NH(C₁-C₃ alkyl), —OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, Ring D is 3 to 10-membered heterocyclyl, R for each occurrence, is independently selected from halogen, OH, CN, —O(C₁-C₃ alkyl), NH₂, NHBoc, NH(C₁-C₃ alkyl), and C₁-C₃ alkyl optionally substituted with 1 to 3 groups selected from halogen, CN, NH₂, NHBoc, and OH, s is an integer selected from 0, 1, and 2; and all other variables not specifically defined herein are as defined in any of the appropriate preceding embodiments.

In a thirty-fifth embodiment, a compound of the disclosure is one of the following structural formula VIIId, VIIIe, or VIIIf:

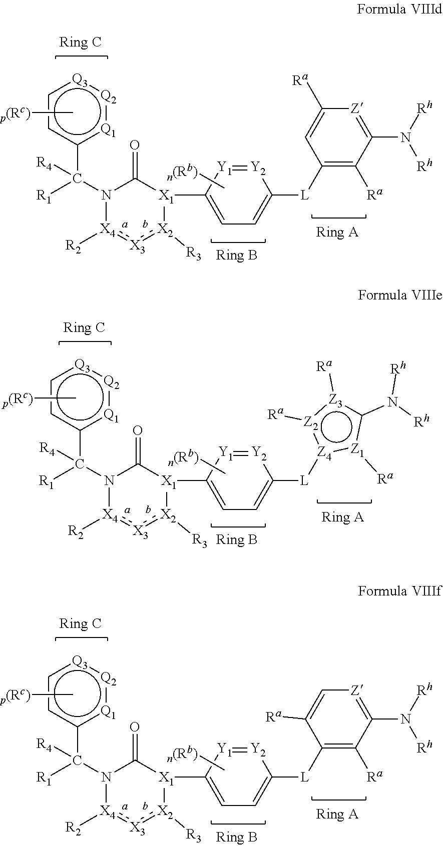

a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z′ is selected from C and N, Z₁, Z₂ and Z₃ are each independently selected from S, O, C and N, Z₄ is selected from C and N, R^{a}, for each occurrence is independently selected from absent, halogen, CN, NO₂, NH₂, NH(C₁-C₃ alkyl), OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, R^{h}, for each occurrence, is independently selected from H, C₁-C₃ alkyl optionally substituted with 1-3 groups selected from halogen and 3 to 4-membered cycloalkyl, 3-6 membered cycloalkyl optionally substituted by 1 to 3 groups selected from halogen and C₁-C₃ alkyl, and 3-6 membered heterocyclyl optionally substituted by 1 to 3 groups selected from halogen and C₁-C₃ alkyl; and all other variables not specifically defined herein are as defined in any of the appropriate preceding embodiments.

In a thirty-sixth embodiment, in a compound, tautomer, a solvate or stereoisomer of the compound or the tautomer, or pharmaceutically acceptable salt of this disclosure,

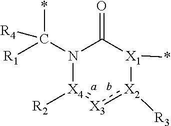

of Formula I is selected from:

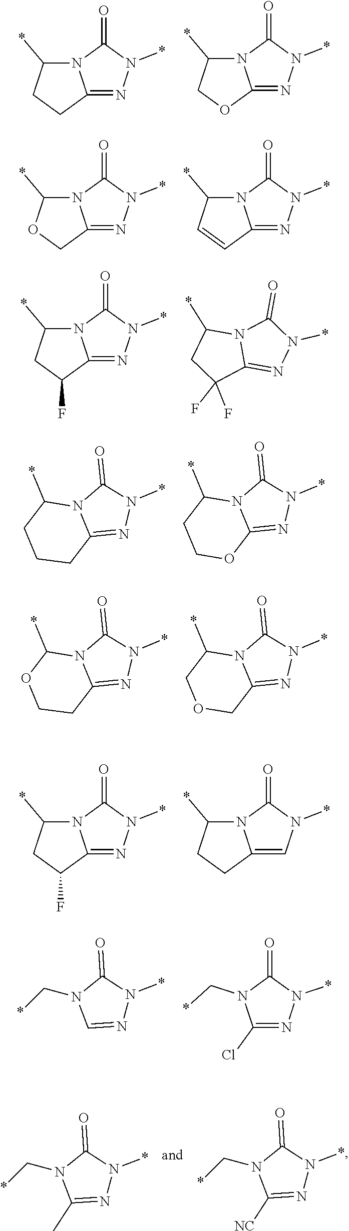

Ring B substituted with n groups of R^{b} is selected from:

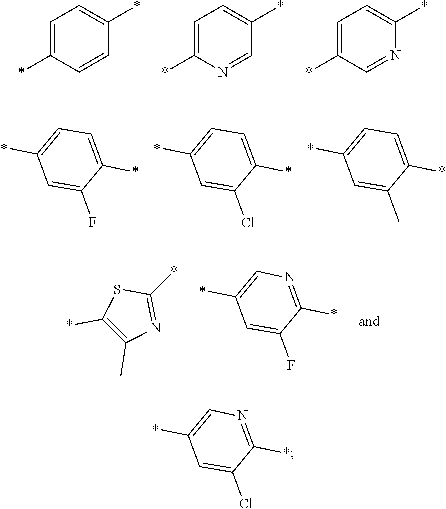

Ring C substituted with p groups of R^{c} is selected from:

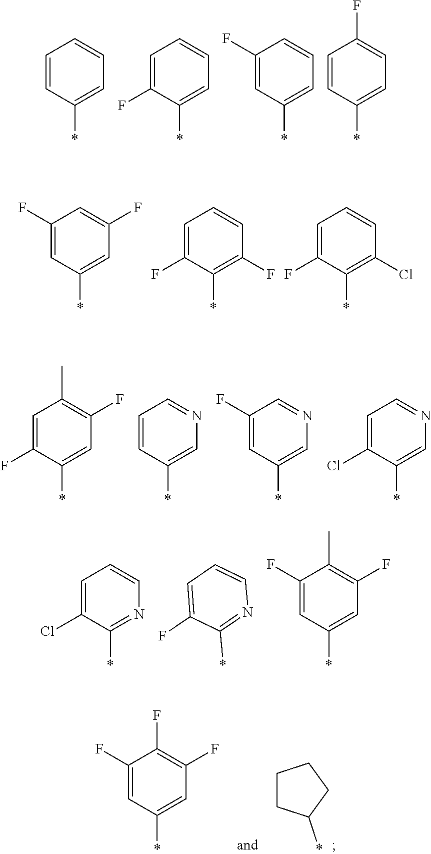

and L is selected from:

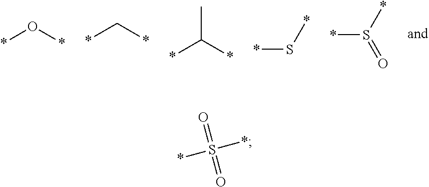

and all other variables not specifically defined herein are as defined in any of the appropriate preceding embodiments.

In a thirty-seventh embodiment, in a compound, tautomer, a solvate or stereoisomer of the compound or the tautomer, or pharmaceutically acceptable salt of this disclosure, Ring A substituted with m groups of R^{a} is selected from:

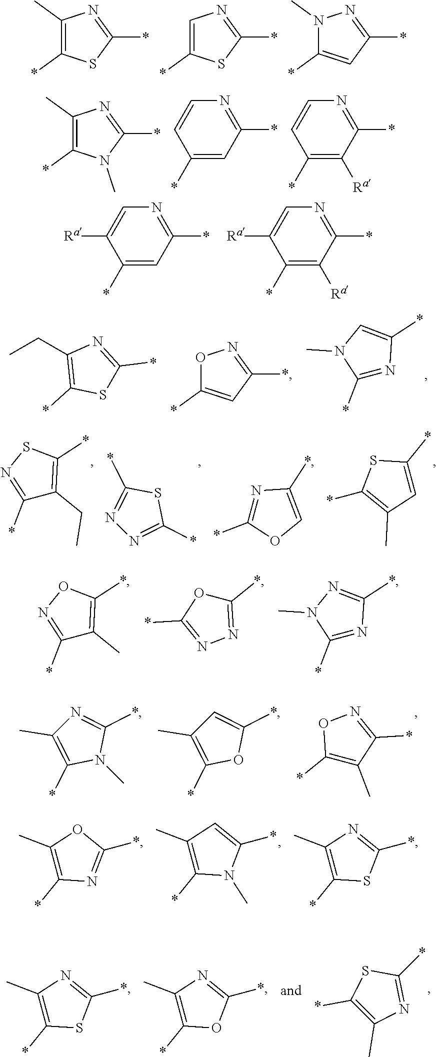

wherein R^{a′}, for each occurrence, is independently selected from F, Cl, —OCH₃, CH₃, NH₂, and CN; L is —O—; the position denoted by the * on the left side of the above structures is connected to L, and the position denoted by the * on the right side of the above structures is connected to an R^{a}; and all other variables not specifically defined herein are as defined in any of the appropriate preceding embodiments.

In a thirty-eighth embodiment, in a compound, tautomer, a solvate or stereoisomer of the compound or the tautomer, or pharmaceutically acceptable salt of this disclosure, Ring A substituted by m groups of R^{a} is selected from:

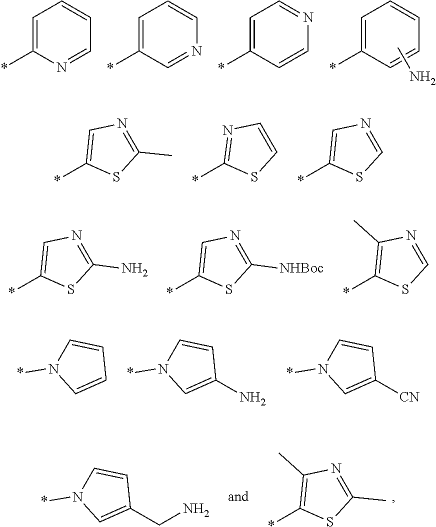

and L is —SO₂—; and all other variables not specifically defined herein are as defined in any of the appropriate preceding embodiments.

In a thirty-ninth embodiment, in a compound, tautomer, a solvate or stereoisomer of the compound or the tautomer, or pharmaceutically acceptable salt of this disclosure,

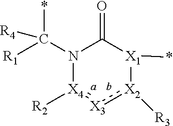

of Formula I is selected from:

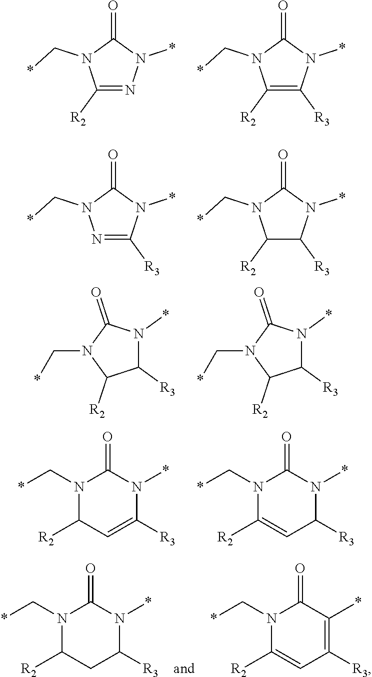

wherein R² is selected from H, halogen, CN, —NH₂, OH, OCH₃, ═O, and C₁ to C₃ alkyl optionally substituted by 1 to 3 groups selected from halogen; and all other variables not specifically defined herein are as defined in any of the appropriate preceding embodiments.

In a fortieth embodiment, in a compound, tautomer, a solvate or stereoisomer of the compound or the tautomer, or pharmaceutically acceptable salt of this disclosure,

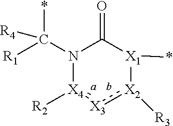

of Formula I is selected from:

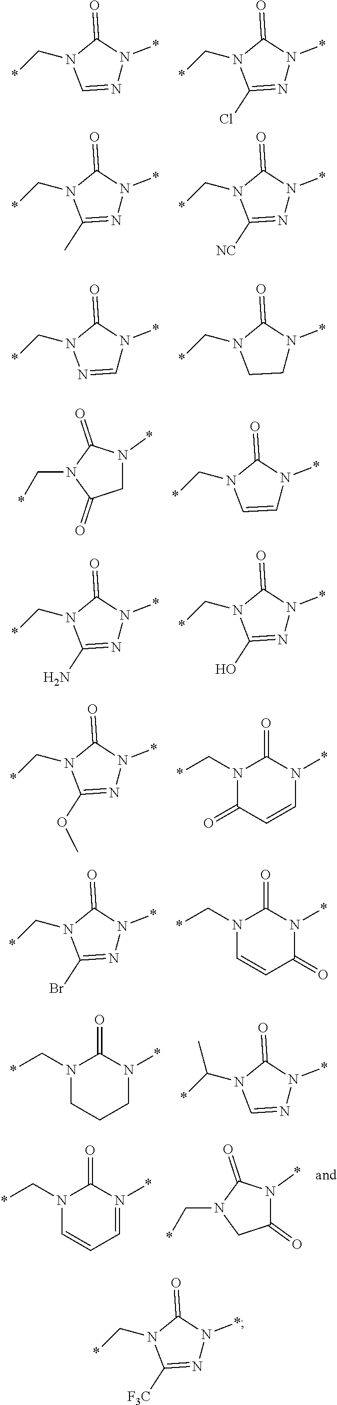

and all other variables not specifically defined herein are as defined in any of the appropriate preceding embodiments.

In a forty-first embodiment, in a compound, tautomer, a solvate or stereoisomer of the compound or the tautomer, or pharmaceutically acceptable salt of this disclosure,

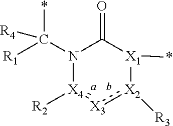

of Formula I is selected from:

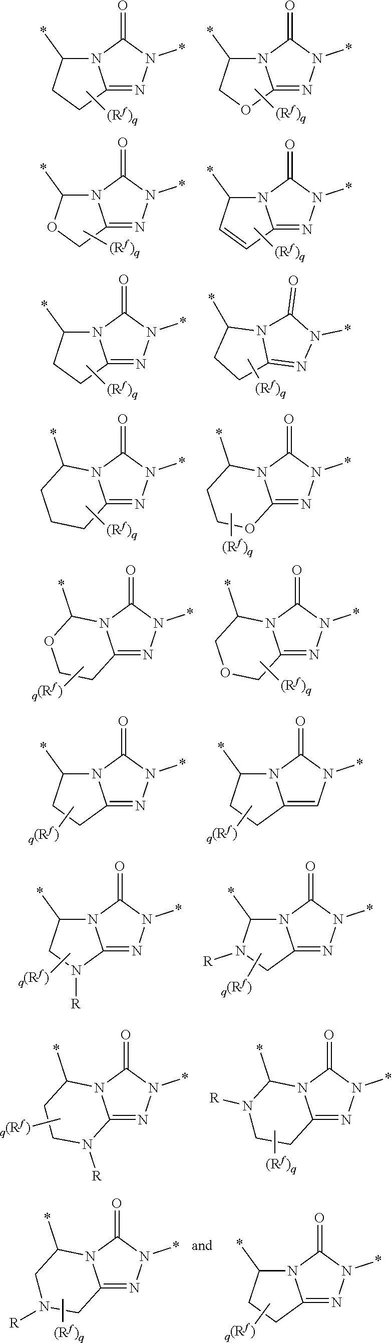

wherein R^{f}, for each occurrence, is independently selected from C₁-C₂ alkyl and halogen, and q is selected from 0, 1, and 2; and all other variables not specifically defined herein are as defined in any of the appropriate preceding embodiments.

In a forty-second embodiment, in a compound, tautomer, a solvate or stereoisomer of the compound or the tautomer, or pharmaceutically acceptable salt of this disclosure,

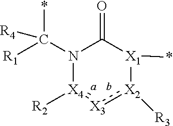

of Formula I is selected from:

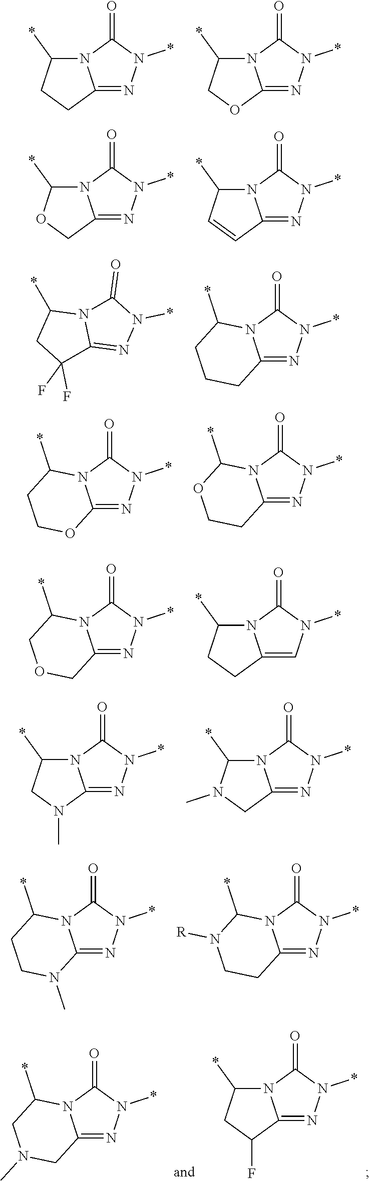

and all other variables not specifically defined herein are as defined in any of the appropriate preceding embodiments.

In a forty-third embodiment, in a compound, tautomer, a solvate or stereoisomer of the compound or the tautomer, or pharmaceutically acceptable salt of this disclosure, Ring A is selected from:

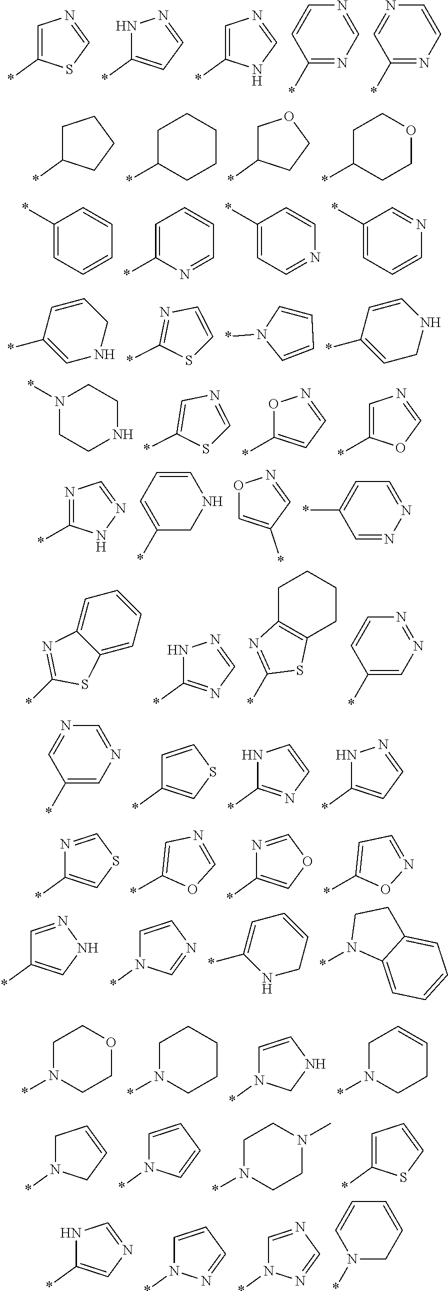

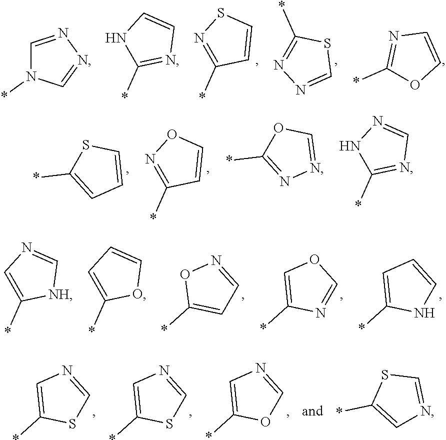

wherein Ring A is substituted with m groups of R^{a}; and all other variables not specifically defined herein are as defined in any of the appropriate preceding embodiments.

In a forty-fourth embodiment, in a compound, tautomer, a solvate or stereoisomer of the compound or the tautomer, or pharmaceutically acceptable salt of this disclosure, Ring A substituted by m groups of R^{a} is selected from:

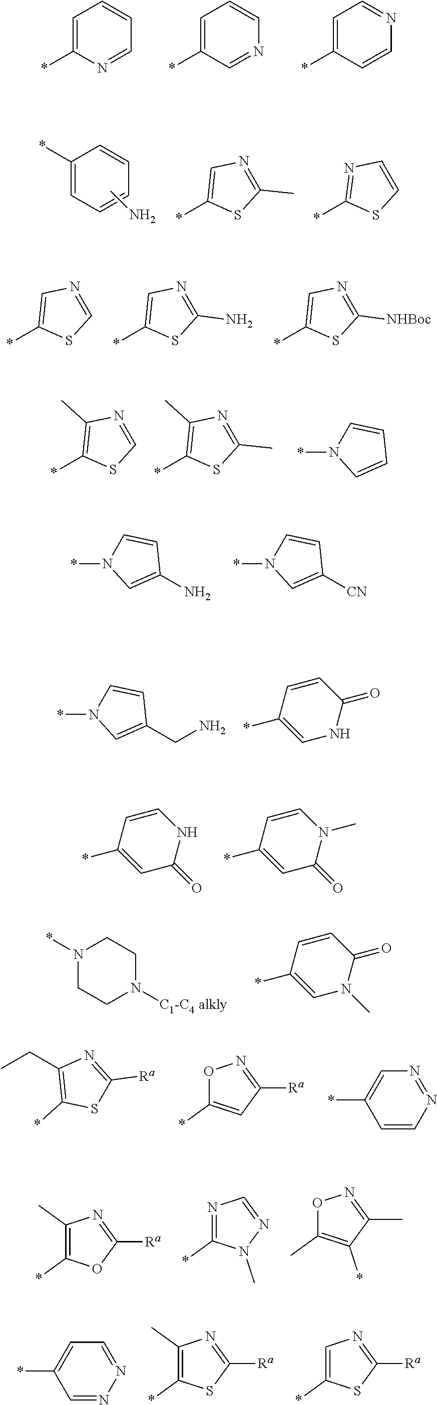

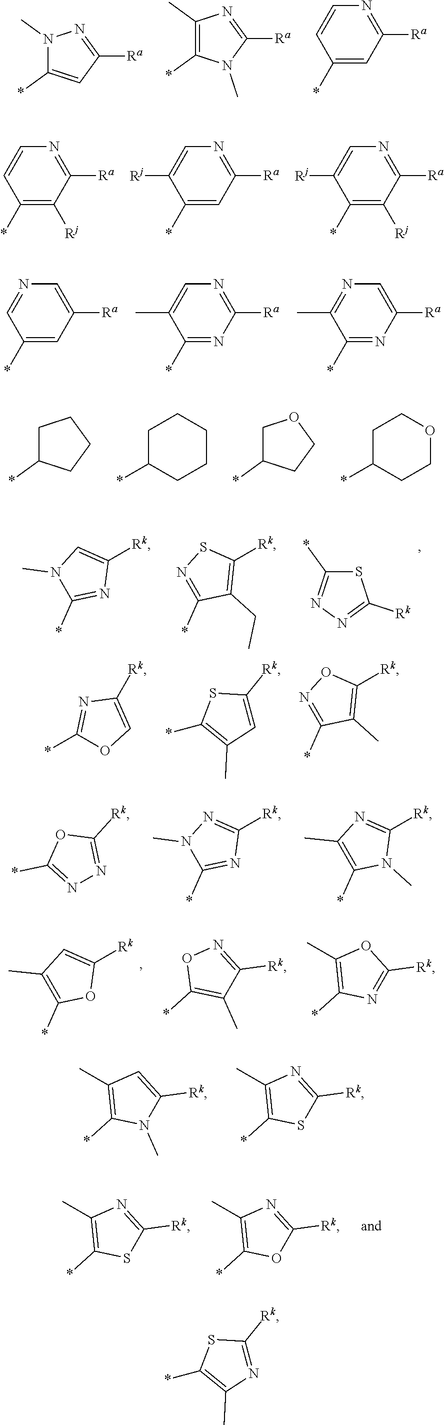

wherein R^{k} is selected from —C(═O)NH₂, —C(═O)NHCH₃, —C(═O)N(CH₃)₂, and, wherein R^{j}, for each occurrence, is independently selected from F, Cl, CH₃, and CN; and all other variables not specifically defined herein are as defined in any of the appropriate preceding embodiments.

In a forty-fifth embodiment, in a compound, tautomer, a solvate or stereoisomer of the compound or the tautomer, or pharmaceutically acceptable salt of this disclosure, Ring B is selected from:

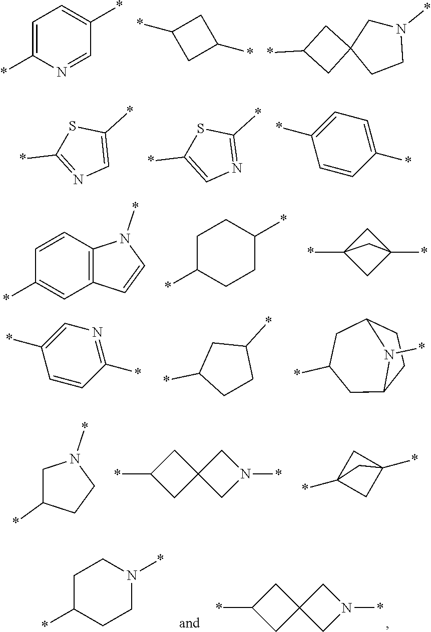

wherein Ring B is substituted with n groups of R^{b}; and all other variables not specifically defined herein are as defined in any of the appropriate preceding embodiments.

In a forty-sixth embodiment, in a compound, tautomer, a solvate or stereoisomer of the compound or the tautomer, or pharmaceutically acceptable salt of this disclosure, Ring B substituted by n groups of R^{b} is selected from:

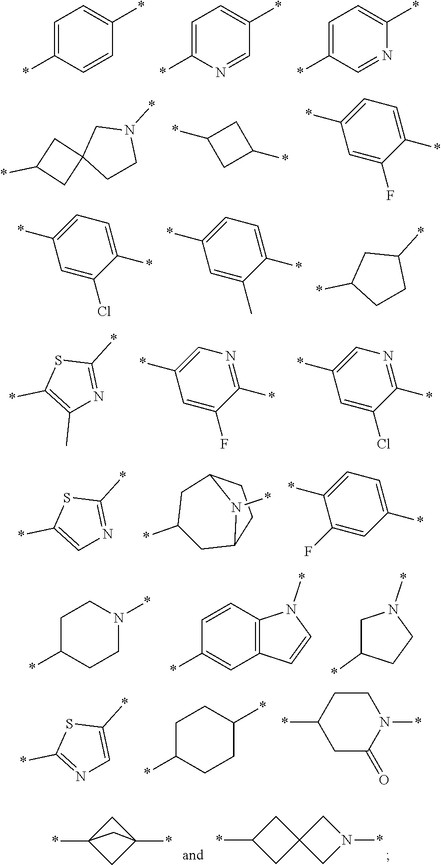

and all other variables not specifically defined herein are as defined in any of the appropriate preceding embodiments.

In a forty-seventh embodiment, in a compound, tautomer, a solvate or stereoisomer of the compound or the tautomer, or pharmaceutically acceptable salt of this disclosure, Ring C is selected from:

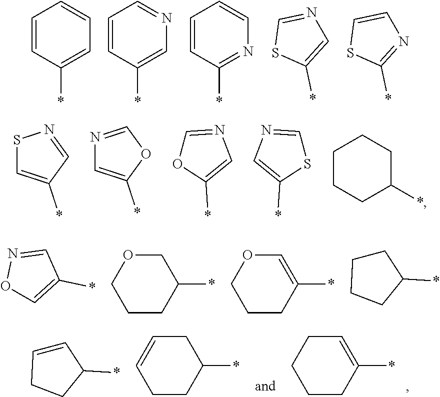

wherein Ring C is substituted with p groups of R^{c}; and all other variables not specifically defined herein are as defined in any of the appropriate preceding embodiments.

In a forty-eighth embodiment, in a compound, tautomer, a solvate or stereoisomer of the compound or the tautomer, or pharmaceutically acceptable salt of this disclosure, Ring C substituted by p groups of R^{c} is selected from:

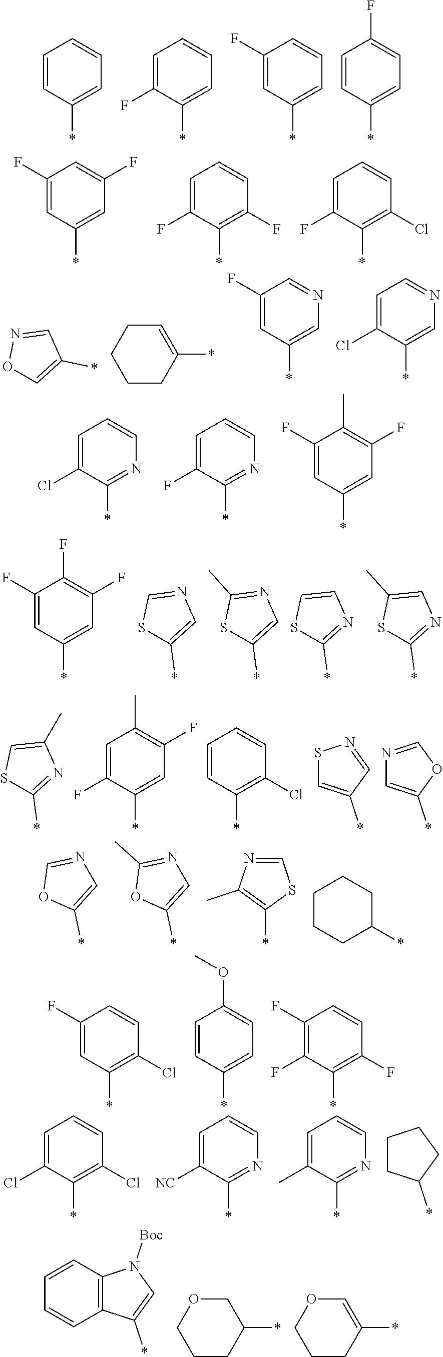

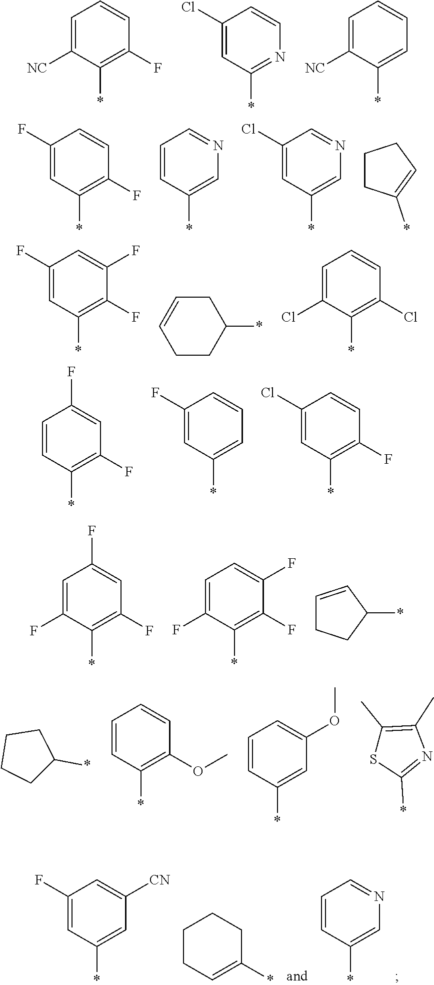

and all other variables not specifically defined herein are as defined in any of the appropriate preceding embodiments.

In a forty-ninth embodiment, in a compound, tautomer, a solvate or stereoisomer of the compound or the tautomer, or pharmaceutically acceptable salt of this disclosure,
R^{a}, for each occurrence, is independently selected from absent; halogen; cyano; ═O; NO₂; C₁ to C₆ alkyl optionally substituted by 1 to 4 groups selected from halogen, CN, —OR^{s}, —C(═O)NR^{p}R^{q}, —C(═O)OR^{s}, —N3, ═NRp, ═NOR^{s}, —NR^{p}R^{q}, 3 to 10-membered cycloalkyl, and 3 to 10-membered heterocyclyl, wherein the 3 to 10-membered cycloalkyl and the 3 to 10-membered heterocyclyl are each optionally substituted by 1 to 4 groups selected from C₁ to C₆ alkyl, halogen, and OR^{s};
C₂ to C₆ alkenyl optionally substituted by 1 to 4 groups selected from halogen, CN, —OR^{s}, —C(═O)NR^{p}R^{q}, —C(═O)OR^{s}, —N3, ═NRp, ═NOR^{s}, —NR^{p}R^{q}, 3 to 10-membered cycloalkyl, and 3 to 10-membered heterocyclyl, wherein the 3 to 10-membered cycloalkyl and the 3 to 10-membered heterocyclyl are each optionally substituted by 1 to 4 groups selected from C₁ to C₆ alkyl, halogen, and OR^{s};
C₂ to C₆ alkynyl optionally substituted by 1 to 4 groups selected from halogen, CN, —OR^{s}, —C(═O)NR^{p}R^{q}, —C(═O)OR^{s}, —N3, ═NRp, =NOR^{s}, —NR^{p}R^{q}, 3 to 10-membered cycloalkyl, and 3 to 10-membered heterocyclyl, wherein the 3 to 10-membered cycloalkyl and the 3 to 10-membered heterocyclyl are each optionally substituted by 1 to 4 groups selected from C₁ to C₆ alkyl, halogen, and OR^{s};
3 to 10-membered cycloalkyl optionally substituted by 1 to 4 groups selected from halogen, CN, OR^{s}, —C(═O)NR^{p}R^{q}, —C(═O)OR, and —NR^{p}R^{q};
3 to 10-membered heterocyclyl optionally substituted by 1 to 4 groups selected from halogen, CN, OR^{s}, —C(═O)NR^{p}R^{q}, —C(═O)OR, and —NR^{p}R^{q};
—C(═O)R^{s};
—C(═O)OR^{s};
—C(═O)(C═O)OR^{s};
—C(═O)NR^{p}R^{q}NR^{p}R^{q};
—C(═O)NR^{p}R^{q}OR^{s};
—C(═O)NR^{p}R^{q};
—NR^{p}R^{q};
—NR^{p}C(═O)R^{s}, wherein R^{p} and R^{s} are defined below in this claim or the R^{p} and R^{s} of NR^{p}C(═O)R^{s} join and form a 5 to 10-membered heterocyclyl;
—NR^{p2}C(═O)OR^{s2}, wherein R^{p2} and R^{s2} are defined below in this claim or the R^{p2} and R^{s2} of —NR^{p2}C(═O)OR^{s2} join and form a 5 to 10-membered heterocyclyl;
—OR^{s}:
wherein:
R^{p} and R^{q}, for each occurrence, are independently selected from hydrogen and C₁-C₆ alkyl, or R^{p} and R^{q} join and form a 3 to 10-membered heterocyclyl, wherein:
the C₁-C₄ alkyl of any one of R^{p} and R^{q} is optionally substituted with 1 to 3 groups selected from halogen, —NR^{p1}C(═O)OR^{s1}, cyano, —OH, —OR^{s1}, —O(C₁ to C₃ alkyl)OR^{s1}, 3 to 10-membered cycloalkyl, 3 to 10-membered heterocyclyl, and phenyl; wherein
the 3 to 10-membered heterocyclyl of any one of R^{p} and R^{q}, and the 3 to 10-membered cycloalkyl and 3 to 10-membered heterocyclyl of the C₁-C₄ alkyl of any one of R^{p} and R^{q}, are each optionally substituted with 1 to 3 groups selected from halogen, CN, ═O, NR^{p1}R^{q1}, OR^{s1}, —NR^{p1}C(═O)R^{s1}, —NR^{p1}C(═O)OR^{s1}, 3 to 10-membered cycloalkyl, and C₁ to C₃ alkyl optionally substituted with C₃-C₄ cycloalkyl;
R^{s}, for each occurrence, is independently selected from hydrogen, C₁-C₆ alkyl, phenyl, 5 to 6-membered heteroaryl, 3 to 10-membered cycloalkyl, and 3 to 10-membered heterocyclyl,
wherein the C₁-C₆ alkyl, phenyl, 5 to 6-membered heteroaryl,
3 to 10-membered cycloalkyl, and 3 to 10-membered heterocyclyl of R^{s} are each optionally substituted with 1 to 3 groups selected from halogen, NR^{p1}R^{g1}, —NR^{p1}C(═O)OR^{s1}, cyano, —OH, —O(C₁ to C₃ alkyl), —O(C₁ to C₃ alkyl)OH, —O(C₁ to C₃ alkyl)O(C₁ to C₃ alkyl), 3 to 10-membered cycloalkyl, 3 to 10-membered heterocyclyl, and phenyl;
R^{p1} and R^{q1}, for each occurrence, are each independently selected from hydrogen and C₁-C₄ alkyl optionally substituted by 1 to 3 groups of halogen, CN, and OH;
R^{s1}, for each occurrence, is independently selected from hydrogen and C₁-C₄ alkyl optionally substituted by 1 to 3 groups of halogen, CN, and OH;
R^{p2}, for each occurrence, is independently selected from hydrogen and C₁-C₄ alkyl optionally substituted by 1 to 3 groups of halogen, CN, and OH;
R^{s2}, for each occurrence, is independently selected from hydrogen and C₁-C₄ alkyl optionally substituted by 1 to 3 groups of halogen, CN, and OH;
and all other variables not specifically defined herein are as defined in any of the appropriate preceding embodiments.

In a fiftieth embodiment, in a compound, tautomer, a solvate or stereoisomer of the compound or the tautomer, or pharmaceutically acceptable salt of this disclosure, R^{a} is selected from absent, NH₂, NO₂, ═O, cyano, I, F, Cl, Br, —CH₃, —CH(CH₃)₂, —CH₂CN, —CF₃, —CH₂OH, —CH₂CH₂OH, —C(CH₃)₂OH, —CH₂C(CH₃)₂OH, —CHF₂, —CHCH₃OH, —CH₂CONH₂, —CH₂OOOH, —CHCH₃NH₂, —CH₂OH, —CH₂CH₂OH, —CH₂N₃, —CH₂NH₂, —CH₂OCH₃,

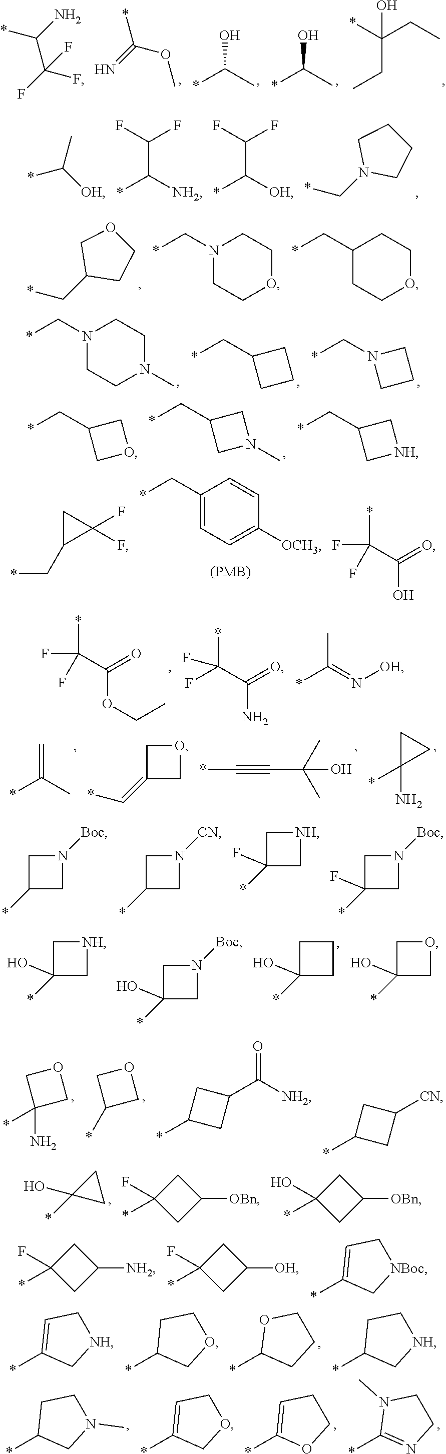

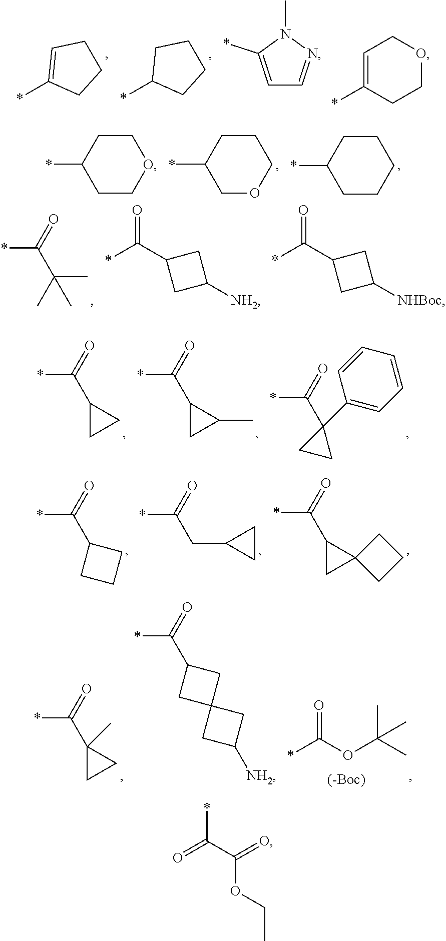

### —C(═O)NHNH₂,

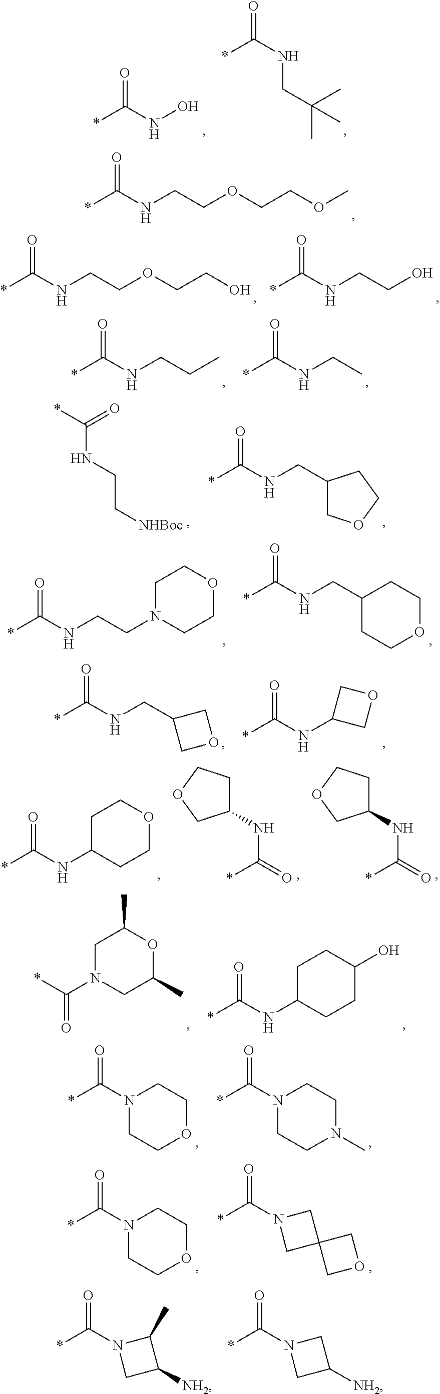

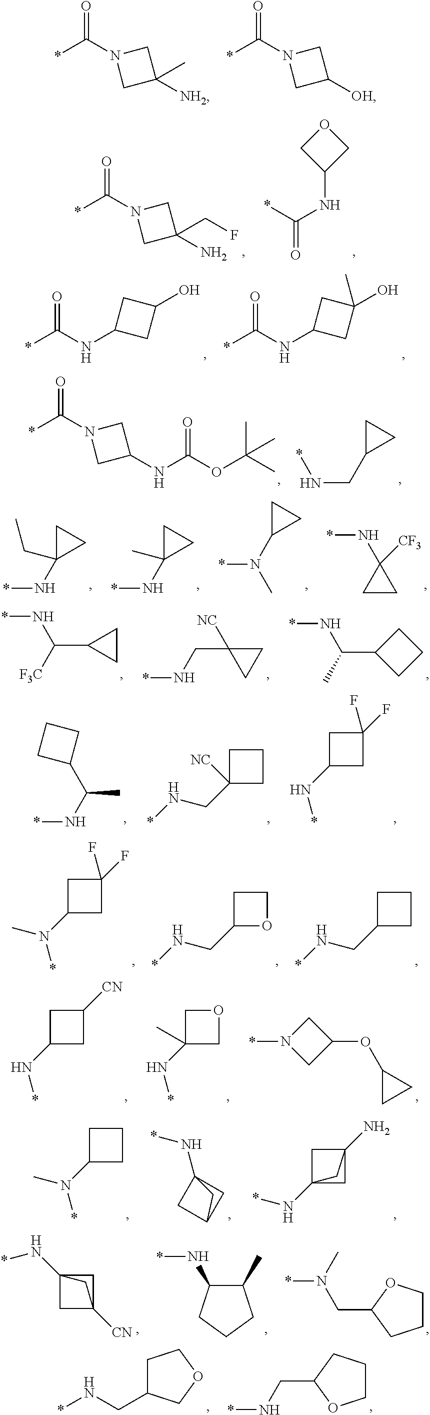

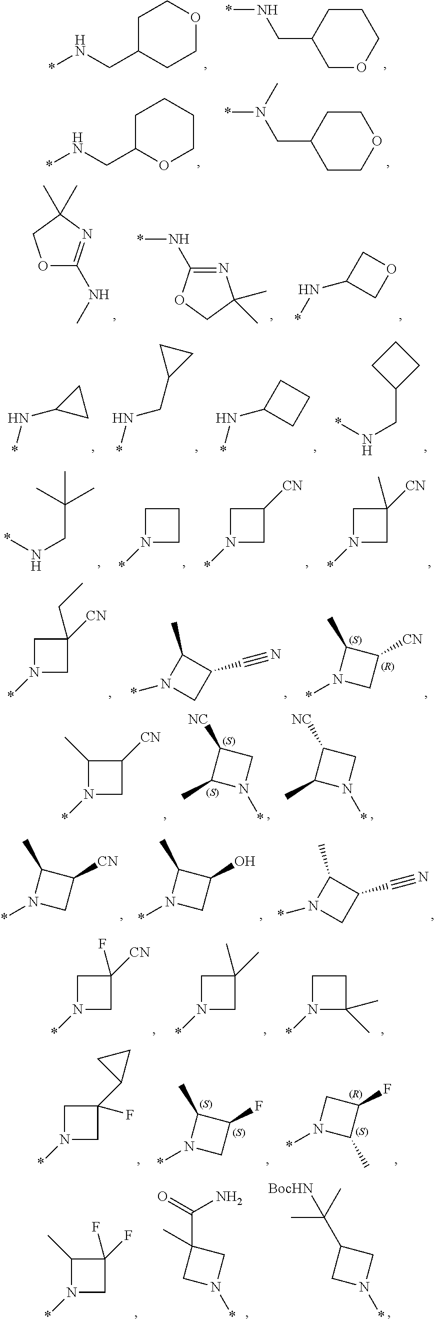

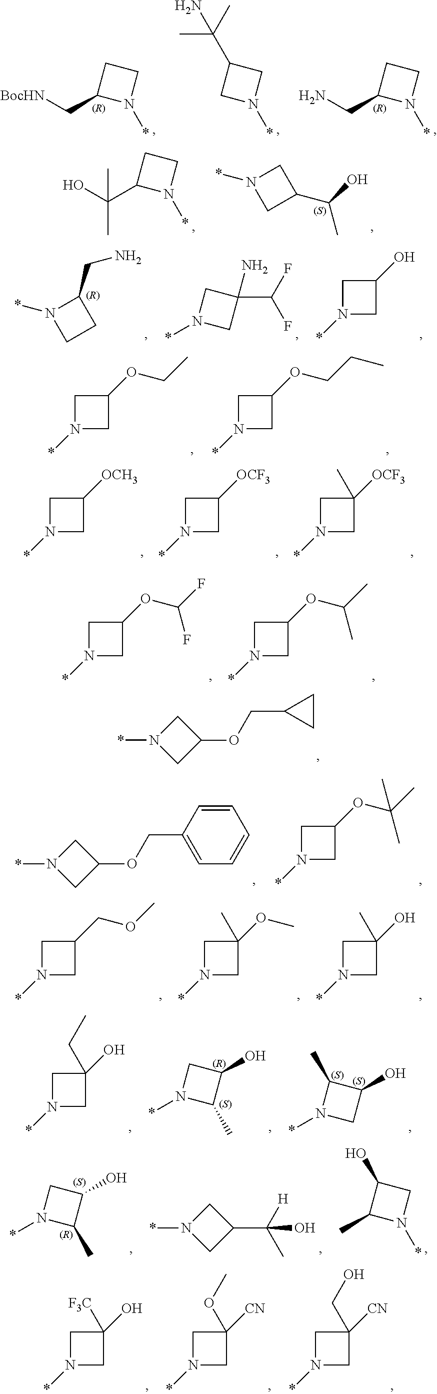

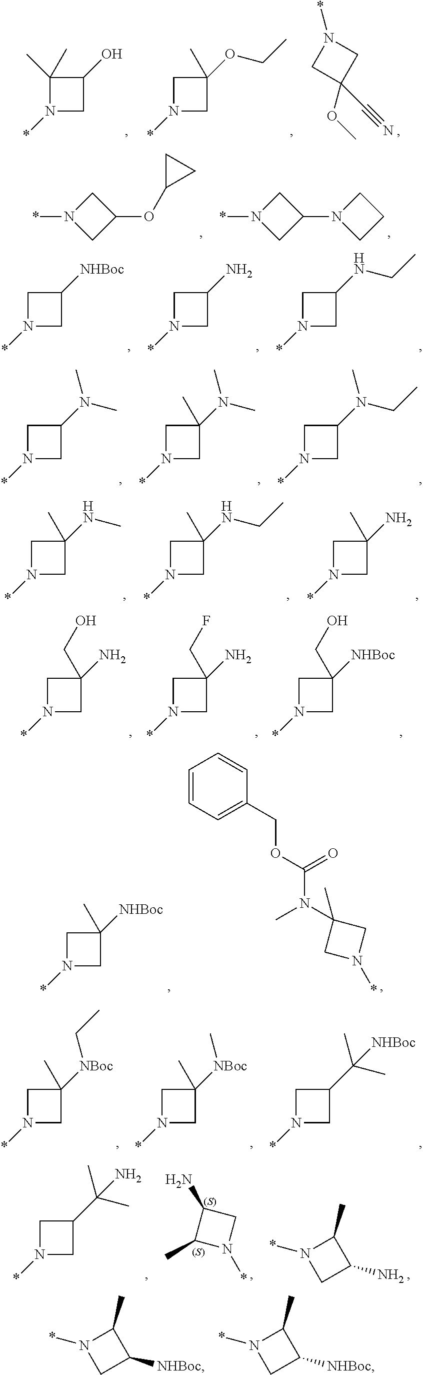

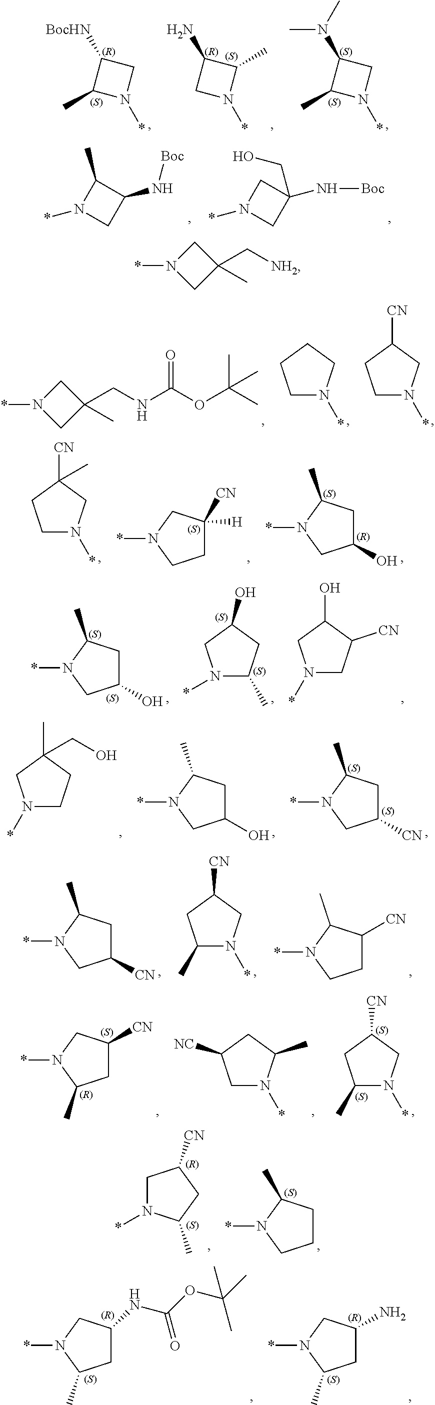

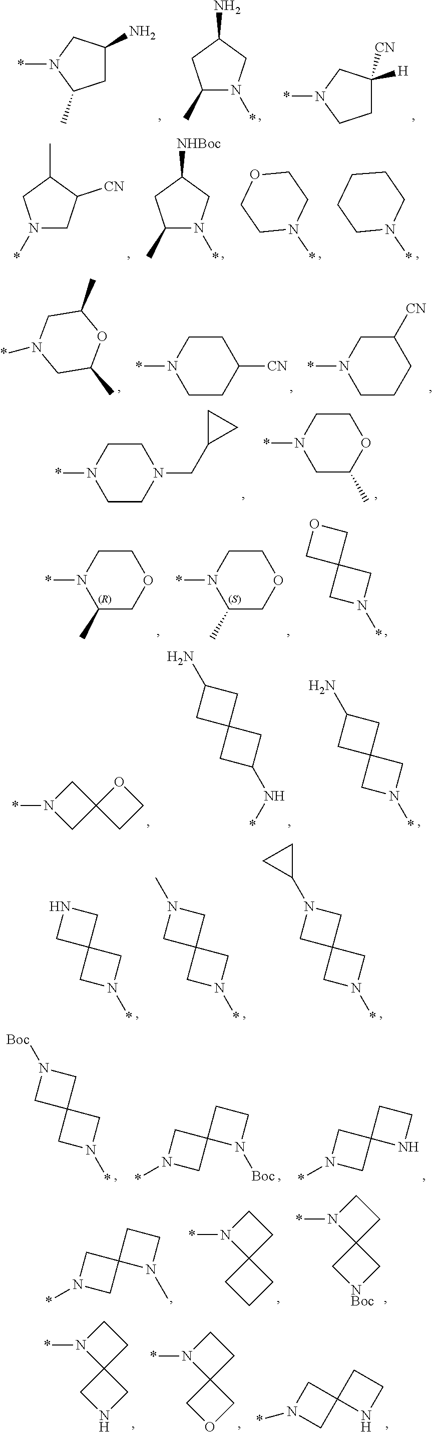

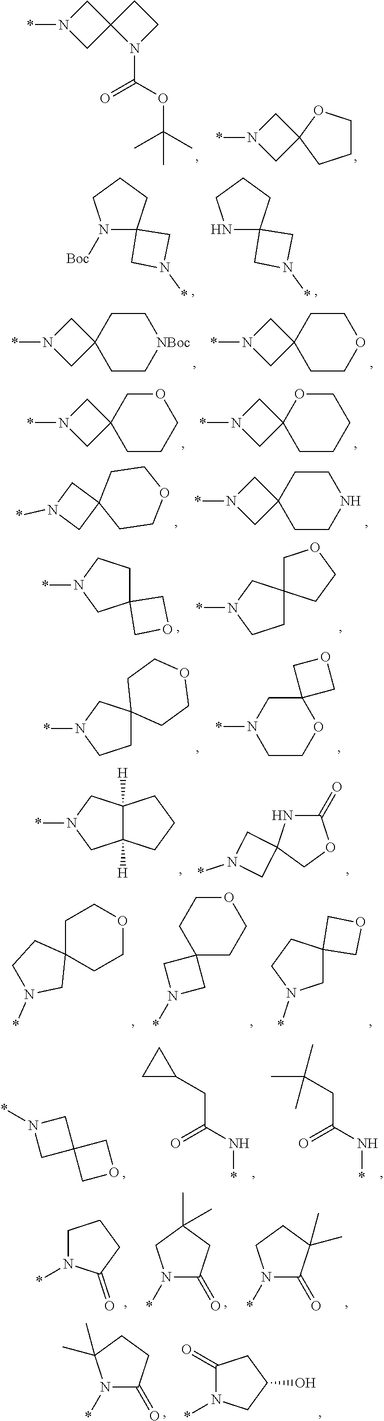

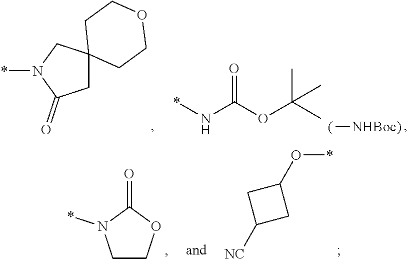

and all other variables not specifically defined herein are as defined in any of the appropriate preceding embodiments.

In a fifty-first embodiment, in a compound, tautomer, a solvate or stereoisomer of the compound or the tautomer, or pharmaceutically acceptable salt of this disclosure, R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, NO₂,

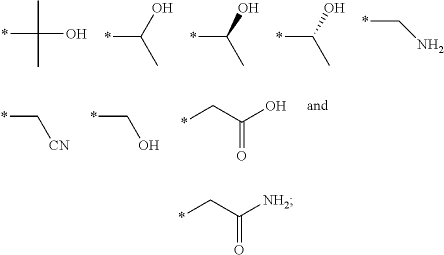

and all other variables not specifically defined herein are as defined in any of the appropriate preceding embodiments.

In a fifty-second embodiment, in a compound, tautomer, a solvate or stereoisomer of the compound or the tautomer, or pharmaceutically acceptable salt of this disclosure, R^{b}, for each occurrence, is independently selected from absent, halogen, ═O, and C₁-C₂ alkyl; and all other variables not specifically defined herein are as defined in any of the appropriate preceding embodiments.

In a fifty-third embodiment, in a compound, tautomer, a solvate or stereoisomer of the compound or the tautomer, or pharmaceutically acceptable salt of this disclosure, R^{b}, for each occurrence, is independently selected from absent, —CH₃, ═O, F, and Cl; and all other variables not specifically defined herein are as defined in any of the appropriate preceding embodiments.

In a fifty-fourth embodiment, in a compound, tautomer, a solvate or stereoisomer of the compound or the tautomer, or pharmaceutically acceptable salt of this disclosure, R^{c}, for each occurrence, is independently selected from absent, C₁-C₃ alkyl, CN, halogen, —OR^{s1}, and —C(═O)OR^{s1}, wherein R^{s1} is H or C₁-C₄ alkyl; and all other variables not specifically defined herein are as defined in any of the appropriate preceding embodiments.

In a fifty-fifth embodiment, in a compound, tautomer, a solvate or stereoisomer of the compound or the tautomer, or pharmaceutically acceptable salt of this disclosure, R^{c}, for each occurrence, is independently selected from absent, CH₃, CN, F, Cl, —OCH₃, and —C(═O)OC(CH₃)₃; and all other variables not specifically defined herein are as defined in any of the appropriate preceding embodiments.

In a fifty-sixth embodiment, in a compound, tautomer, a solvate or stereoisomer of the compound or the tautomer, or pharmaceutically acceptable salt of this disclosure, R₂ is selected from H, halogen, CN, OR^{s1}, —NR^{p1}R^{q1}, ═O, and C₁ to C₂ alkyl optionally substituted by 1 to 3 groups selected from halogen, wherein R^{s1}, R^{p1}, and R^{q1} are independently selected from H and CH₃; and all other variables not specifically defined herein are as defined in any of the appropriate preceding embodiments.

In a fifty-seventh embodiment, in a compound, tautomer, a solvate or stereoisomer of the compound or the tautomer, or pharmaceutically acceptable salt of this disclosure, R₂ is selected from H, CH₃, CF₃, CN, F, Cl, Br, OH, OCH₃, NH₂, and ═O; and all other variables not specifically defined herein are as defined in any of the appropriate preceding embodiments.

In a fifty-eighth embodiment, in a compound, tautomer, a solvate or stereoisomer of the compound or the tautomer, or pharmaceutically acceptable salt of this disclosure, R₁ and R₂ join to form a 5- to 6-membered ring optionally substituted by 1 to 2 groups selected from halogen and C₁ to C₂ alkyl optionally substituted by 1 to 2 groups selected from halogen; and all other variables not specifically defined herein are as defined in any of the appropriate preceding embodiments.

In a fifty-ninth embodiment, in a compound, tautomer, a solvate or stereoisomer of the compound or the tautomer, or pharmaceutically acceptable salt of this disclosure, R₁ and R₂ join to form a 5- to 6-membered ring optionally substituted by 1 to 2 groups selected from F and CH₃; and all other variables not specifically defined herein are as defined in any of the appropriate preceding embodiments.

In a sixtieth embodiment, in a compound, tautomer, a solvate or stereoisomer of the compound or the tautomer, or pharmaceutically acceptable salt of this disclosure, L is selected from —N(R^{x})—, —(CH₂)ᵤO(CH₂)ᵤ—, —(CH₂)ᵤS(═O)—(CH₂)ᵤ—, —S(═O)(═NR^{x})—, —(NR^{x})S(═O)_{w}—, —S(═O)_{w}(NR^{x})—, —C(═O)—, and C₁-C₃ alkylene, wherein the C₁-C₃ alkylene of L is optionally substituted by 1 to 2 groups selected from OH, C₁-C₃ alkyl, and ═CHR^{x}, wherein the C₁-C₃ alkyl of the C₁-C₃ alkylene of L optionally join to form a C₃-C₄ cycloalkyl, wherein R^{x} is selected from H and C₁-C₂ alkyl, u, for each occurrence, is independently 0 or 1; and all other variables not specifically defined herein are as defined in in any of the appropriate preceding embodiments.

In a sixty-first embodiment, in a compound, tautomer, a solvate or stereoisomer of the compound or the tautomer, or pharmaceutically acceptable salt of this disclosure, L is selected from

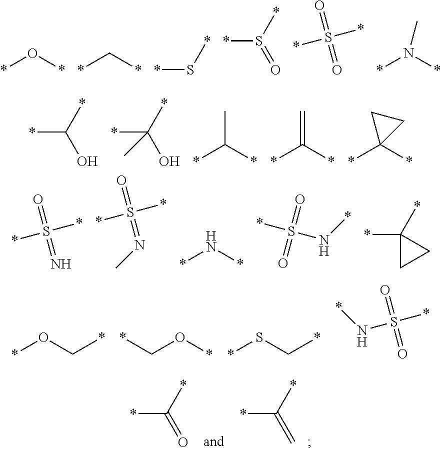

and all other variables not specifically defined herein are as defined in any of the appropriate preceding embodiments.

In a sixty-second embodiment, in a compound, tautomer, a solvate or stereoisomer of the compound or the tautomer, or pharmaceutically acceptable salt of this disclosure, L is selected from

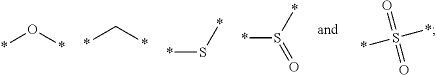

and all other variables not specifically defined herein are as defined in any of the appropriate preceding embodiments.

In certain embodiments, the at least one compound of the disclosure is selected from Compounds 1 to 702, a tautomer thereof, a solvate or stereoisomer of the compound or the tautomer, or a pharmaceutically acceptable salt of the foregoing.

Another aspect of the disclosure provides a pharmaceutical composition comprising at least one compound selected from a compound of the Formulae disclosed herein (e.g., Compounds 1 to 702) disclosed herein, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of the Formulae disclosed herein (e.g., Compounds 1 to 702) disclosed herein, a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of the Formulae disclosed herein (e.g., Compounds 1 to 702) disclosed herein, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water-soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used in the aforementioned form or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment. For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e., the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

In another aspect of this disclosure, a compound, tautomer, hydrate, stereoisomer, or pharmaceutically acceptable salt as described herein, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof, is for use in treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy. In some embodiments, the disease or condition is mediated by receptor-interacting protein 1 (RIP1) signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection.

In another aspect, disclosed herein is a compound, tautomer, hydrate, stereoisomer, or pharmaceutically acceptable salt as described herein, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof, for use as a medicament.

In another aspect, disclosed herein is use of a compound, tautomer, hydrate, stereoisomer, or pharmaceutically acceptable salt as described herein, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof, for the manufacture of a medicament for treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy. In some embodiments, the disease or condition is mediated by RIP1 signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection. In yet another aspect, disclosed herein is a method of treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy in a subject, comprising administering a therapeutically effective amount of a compound, tautomer, a hydrate or stereoisomer of the compound or the tautomer, or pharmaceutically acceptable salt as described herein, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof. In some embodiments, the disease or condition is mediated by RIP1 signaling. In some embodiments, the disease or condition is selected from ulcerative colitis, Crohn's disease, psoriasis, rheumatoid arthritis, ALS, Alzheimer's disease, and a viral infection.

In a further aspect of this disclosure, a compound, tautomer, hydrate, stereoisomer, or pharmaceutically acceptable salt as described herein, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof, is for use in treating a disease or condition mediated by RIP1 signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection. In another aspect, disclosed herein is use of a compound, tautomer, a hydrate or stereoisomer of the compound or the tautomer, or pharmaceutically acceptable salt as described herein, including a compound of the Formulae disclosed herein, Compounds 1 to 702 a tautomer thereof, a hydrate or stereoisomer of the compound or the tautomer, or a pharmaceutically acceptable salt of the foregoing, or a pharmaceutical composition thereof, for the manufacture of a medicament for treating a disease or condition mediated by RIP1 signaling. In some embodiments, the disease or condition is selected from ulcerative colitis, Crohn's disease, psoriasis, rheumatoid arthritis, ALS, Alzheimer's disease, and a viral infection. In yet another aspect, disclosed herein is a method of treating a disease or condition mediated by RIP1 signaling in a subject, comprising administering a therapeutically effective amount of a compound, tautomer, a hydrate or stereoisomer of the compound or the tautomer, or pharmaceutically acceptable salt as described herein, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof. In some embodiments, the disease or condition is selected from ulcerative colitis, Crohn's disease, psoriasis, rheumatoid arthritis, ALS, Alzheimer's disease, and a viral infection.

In another aspect of this disclosure, a compound, tautomer, a hydrate or stereoisomer of the compound or the tautomer, or pharmaceutically acceptable salt as described herein, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof, is for use in mediating, e.g., inhibiting, RIP1 by contacting the RIP1 protein or a fragment thereof (e.g., kinase domain, intermediate domain, and/or death domain) with the compound, tautomer, a hydrate or stereoisomer of the compound or the tautomer, pharmaceutically acceptable salt, or pharmaceutical composition. In yet another aspect, disclosed herein is a method of inhibiting RIP1, comprising contacting the RIP1 protein or a fragment thereof (e.g., kinase domain, intermediate domain, and/or death domain) with a compound, tautomer, a hydrate or stereoisomer of the compound or the tautomer, or pharmaceutically acceptable salt as described herein to a subject, including a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof.

A compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof may be administered once daily, twice daily, or three times daily, for example, for the treatment of a disease or condition as described above, e.g., a disease or condition selected from an inflammatory disease, an immune disease (e.g., an autoimmune disease), an allergic disease, transplant rejection, a necrotic cell disease, a neurodegenerative disease, CNS disease, ischemic brain injury, an ocular disease, an infectious disease, and a malignancy, including those mediated by RIP1 signaling; a disease or condition selected from ulcerative colitis, Crohn's disease, psoriasis, rheumatoid arthritis, ALS, Alzheimer's disease, and a viral infection, including those mediated by RIP1 signaling; a disease or condition mediated by RIP1 signaling.

In some embodiments, 2 mg to 1500 mg or 5 mg to 1000 mg of a compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof are administered once daily, twice daily, or three times daily.

A compound of the Formulae disclosed herein, Compounds 1 to 702, a tautomer thereof, a hydrate or stereoisomer of the compound or the tautomer, or a pharmaceutically acceptable salt of the foregoing, or a pharmaceutical composition thereof may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, hydrates, stereoisomers, and pharmaceutically acceptable salt disclosed herein. Generally, administration is adjusted to achieve a therapeutic dosage of about 0.1 to 50, preferably 0.5 to 10, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

The subject compositions may also be coformulated and/or coadministered with a different compound to treat applicable indications, or to treat programmed cell death. In some embodiments, applicable indications include brain injury, neurodegenerative diseases, viral infections, immune tolerance, and cancer, e.g., to promote tumor immunity in pancreatic cancer and melanoma.

### EXAMPLES

In order that the disclosure described herein may be more fully understood, the following examples are disclosed herein. It should be understood that these examples are for illustrative purposes only and are not to be construed as limiting this disclosure in any way.

### Example 1. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following synthetic schemes for compounds 1 to 702 as representative examples of Formula I.

The intermediates 5-phenyl-2,5,6,7-tetrahydro-3H-pyrrolo[2,1-c][1,2,4]triazol-3-one, methyl 5-(2-fluoro-4-(3-oxo-5-phenyl-6,7-dihydro-3H-pyrrolo[2,1-c][1,2,4]triazol-2(5H)-yl)phenoxy)-4-methylthiazole-2-carboxylate and 2-(3-fluoro-4-hydroxyphenyl)-5-phenyl-2,5,6,7-tetrahydro-3H-pyrrolo[2,1-c][1,2,4]triazol-3-one were purified by chiral HPLC to give two stereoisomers. They were used as starting materials to synthesize the corresponding target compounds.

### Example 1: Method 1

### 2-(4-((2-bromo-4-methylthiazol-5-yl)oxy)-3-fluorophenyl)-4-(2,6-difluorobenzyl)-2,4-dihy dro-3H-1,2,4-triazol-3-one

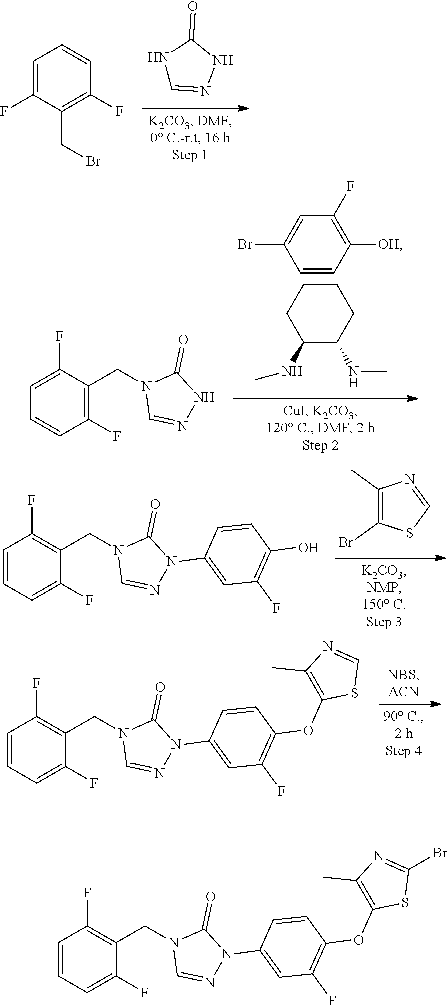

### Step 1: 4-(2,6-difluorobenzyl)-2,4-dihydro-3H-1,2,4-triazol-3-one

2,4-dihydro-3H-1,2,4-triazol-3-one (41.06 g, 0.483 mol) was dissolved in 300 mL DMF. K₂CO₃ (138 g, 1 mol) was added. 2-(bromomethyl)-1,3-difluorobenzene (100 g, 0.483 mol) in 200 mL DMF was added dropwise to the solution at 0° C. The reaction mixture was stirred at room temperature for 16 h. The solvent was evaporated to dryness to obtain a residue. Water was added to the residue and extracted with EA (2 L X 3). The organic layers were combined and evaporated to dryness to give a crude product. It was triturated with tert-Butyl methyl ether to give 59 g white solid. Yield: 57.9%. LC-MS (m/z): 212.2 [M+H]⁺.

### Step 2:

### 4-(2,6-difluorobenzyl)-2-(3-fluoro-4-hydroxyphenyl)-2,4-dihydro-3H-1,2,4-triazol-3-one

4-(2,6-difluorobenzyl)-2,4-dihydro-3H-1,2,4-triazol-3-one (20 g, 0.094 mol), and 4-bromo-2-fluorophenol (17.8 g, 0.094 mol) were mixed in 400 mL 1,4-dioxane. K₂CO₃ (39.0 g, 0.28 mol), CuI (2.66 g, 0.014 mol) and DMDACH ((1S,2S)—N¹,N²-dimethylcyclohexane-1,2-diamine) (4 g, 0.028 mol) were added. The reaction mixture was refluxed for 16 h under N₂. It was cooled and filtered. The solid was washed with DCM. The organic layers were combined and evaporated to dryness to obtain a residue. The residue was purified by column chromatography (PE/EA=9/1 to 4/1) to give 4-(2,6-difluorobenzyl)-2-(3-fluoro-4-hydroxyphenyl)-2,4-dihydro-3H-1,2,4-triazol-3-one as beige solid (10 g, yield: 33.1%). LC-MS (m/z): 322.3 [M+H]⁺.

### Step 3: 4-(2,6-difluorobenzyl)-2-(3-fluoro-4-((4-methylthiazol-5-yl)oxy)phenyl)-2,4-dihydro-3H-1,2,4-triazol-3-one

To a solution of 5-bromo-4-methylthiazole (4.96 g, 0.0279 mol) in NMP (50 mL) was added 4-(2,6-difluorobenzyl)-2-(3-fluoro-4-hydroxyphenyl)-2,4-dihydro-3H-1,2,4-triazol-3-on e (6.87 g, 0.0214 mol) and potassium carbonate (5.92 g, 0.0429 mol) under Ar. The reaction mixture was stirred at 150° C. for 2 h. Water was added and the reaction solution was extracted with EtOAc (300 mL×3). The organic layers were combined and washed with water (150 mL) and brine. The organic layer was dried with MgSO₄, filtered and evaporated to dryness to obtain a residue. The residue was purified by column chromatography (PE/EA=9/1 to 4/1) to give 4-(2,6-difluorobenzyl)-2-(3-fluoro-4-((4-methylthiazol-5-yl)oxy)phenyl)-2,4-dihydro-3H-1,2,4-triazol-3-one was obtained as a light-yellow solid (5.9 g, yield: 66.0%). LC-MS (m/z): 419.1 [M+H]⁺.

### Step 4:

### 2-(4-((2-bromo-4-methylthiazol-5-yl)oxy)-3-fluorophenyl)-4-(2,6-difluorobenzyl)-2,4-dihy dro-3H-1,2,4-triazol-3-one

To a solution of 4-(2,6-difluorobenzyl)-2-(3-fluoro-4-hydroxyphenyl)-2,4-dihydro-3H-1,2,4-triazol-3-one (2 g, 4.78 mmol) in MeCN (50 mL) was added 1-bromopyrrolidine-2,5-dione (1.70 g, 9.56 mmol). The reaction was stirred at 90° C. for 2 h. The reaction progress was monitored by LCMS. When the starting material was consumed, the reaction solution was concentrated to dryness and the residue was extracted with EtOAc (100 md×3). The organic layers were combined and washed with water (50 mL) and brine. The organic layer was dried with MgSO₄, filtered and evaporated to dryness to obtain a residue. The residue was purified by column chromatography (PE/EA=9/1 to 1/1) to give 2-(4-((2-bromo-4-methylthiazol-5-yl)oxy)-3-fluorophenyl)-4-(2,6-difluorobenzyl)-2,4-dihydro-3H-1,2,4-triazol-3-one as light-yellow solid(1.82 g, yield: 76.56R). LC-MS (m/z): 497.1, 499.1. [M+H]⁺. ¹H NMR (400 MHz, DMSO-d₆) δ 8.35 (s, 1H), 7.88 (dd, J=12.4, 2.4 Hz, 1H), 7.71-7.64 (m, 1H), 7.48 (tt, J 8.4, 6.8 Hz, 1H), 7.33 (t, J=9.2 Hz, 1H), 7.21-7.10 (i, 2H), 4.98 (s, 2H), 2.21 (s, 3H).

### Examples (Compounds) 2-9 were Synthesized Using a Similar Method to that Used in Example 1

| Example | | | Appear- | | MS |
|---|---|---|---|---|---|
| (Compound) | | | ance | | (m/z) |
| No. | Activi- | | and | ¹H NMR Data | [M + |
| Method | ty | Structure and Name | Yield | | H]⁺ |
| Example 1 Method 1 | ++++ | 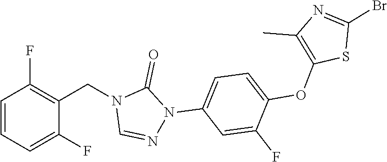 [SMILES: Cc1nc(Br)sc1Oc1ccc(-n2ncn(Cc3c(F)cccc3F)c2=O)cc1F] | light- yellow solid, yield: 76.6% | ¹H NMR (400 MHz, DMSO-d₆) δ 8.35 (s, 1H), 7.88 (dd, J = 12.4, 2.4 Hz, 1H), 7.71-7.64 (m, 1H), 7.48 (tt, J = 8.4, 6.8 Hz, 1H), 7.33 (t, J = 9.2 Hz, 1H), 7.21-7.10 (m, 2H), 4.98 (s, 2H), 2.21 (s, 3H). | 497.1, 499.1 |
| | | 2-(4-((2-bromo-4-methylthiazol-5- | | | |
| | | yl)oxy)-3-fluorophenyl)-4-(2,6-difluoro- | | | |
| | | benzyl)-2,4-dihydro-3H-1,2,4-tri- | | | |
| | | azol-3-one | | | |
| Example 2 Method 1 | ++++ | 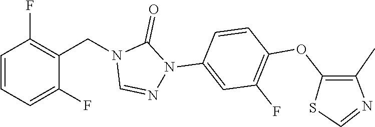 [SMILES: Cc1ncsc1Oc1ccc(-n2ncn(Cc3c(F)cccc3F)c2=O)cc1F] | brown solid, yield: 70% | ¹H NMR (400 MHz, CDCl₃) δ 8.44 (s, 1H), 7.91 (dd, J = 12.0, 2.4 Hz, 1H), 7.73 (ddd, J = 9.2, 2.4, 1.6 Hz, 1H), 7.58 (s, 1H), 7.41-7.33 (m, 1H), 7.03-6.94 (m, 3H), 4.97 (s, 2H), 2.37 (s, 3H). | 419.4 |
| | | 4-(2,6-difluorobenzyl)-2-(3-fluoro- | | | |
| | | 4-((4-methylthiazol-5-yl)oxy)phenyl)- | | | |
| | | 2,4-dihydro-3H-1,2,4-triazol-3-one | | | |
| Example 3 Method 1 Single unknown stereoisomer | ++++ | 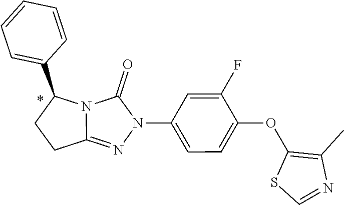 [SMILES: Cc1ncsc1Oc1ccc(-n2nc3n(c2=O)[C@H](c2ccccc2)CC3)cc1F] | white solid, yield: 37.6% | ¹H NMR (400 MHz, Chloroform-d) δ 8.40 (s, 1H), 7.88 (dd, J = 12.4, 2.4 Hz, 1H), 7.69 (dt, J = 9.2, 2.0 Hz, 1H), 7.43-7.31 (m, 3H), 7.26-7.23 (m, 2H), 6.98 (t, J = 8.8 Hz, 1H), 5.28 (dd, J = 7.6, 4.0 Hz, 1H), 3.12- 2.97 (m, 2H), 2.97-2.85 (m, 1H), 2.60-2.46 (m, 1H), 2.36 (s, 3H). | 409.2 |
| | | (S)-2-(3-fluoro-4-((4-methylthiazol- | | | |
| | | 5-yl)oxy)phenyl)-5-phenyl-2,5,6,7- | | | |
| | | tetrahydro-3H-pyrrolo[2,1-c][1,2,4] | | | |
| | | triazol-3-one | | | |
| Example 4 Method 1 Single unknown stereoisomer | ++++ | 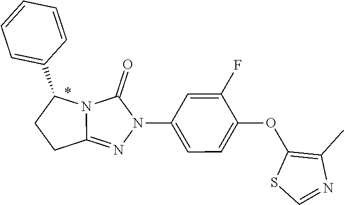 [SMILES: Cc1ncsc1Oc1ccc(-n2nc3n(c2=O)[C@@H](c2ccccc2)CC3)cc1F] | white solid, yield: 39.1% | ¹H NMR (400 MHz, Chloroform-d) δ 8.42 (s, 1H), 7.88 (dd, J = 12.4, 2.4 Hz, 1H), 7.69 (dt, J = 9.2, 2.0 Hz, 1H), 7.44-7.31 (m, 3H), 7.26-7.23 (m, 2H), 6.98 (t, J = 8.8 Hz, 1H), 5.28 (dd, J = 7.6, 4.0 Hz, 1H), 3.13- 2.98 (m, 2H), 2.98-2.86 (m, 1H), 2.61-2.44 (m, 1H), 2.36 (s, 3H). | 409.2 |
| | | (R)-2-(3-fluoro-4-((4-methylthiazol- | | | |
| | | 5-yl)oxy)phenyl)-5-phenyl-2,5,6,7- | | | |
| | | tetrahydro-3H-pyrrolo[2,1-c][1,2,4] | | | |
| | | triazol-3-one | | | |
| Example 5 Method 1 | ++++ | 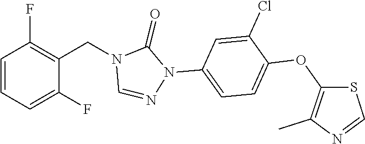 [SMILES: Cc1ncsc1Oc1ccc(-n2ncn(Cc3c(F)cccc3F)c2=O)cc1Cl] | Red solid, yield: 31.5% | ¹H NMR (400 MHz, DMSO-d₆) δ 8.75 (s, 1H), 8.34 (s, 1H), 8.07 (d, J = 2.4 Hz, 1H), 7.80 (dd, J = 9.2, 2.8 Hz, 1H), 7.48 (tt, J = 8.4, 6.8 Hz, 1H), 7.20-7.10 (m, 3H), 4.98 (s, 2H), 2.24 (s, 3H). | 435.2 |
| | | 2-(3-chloro-4-((4-methylthiazol-5-yl) | | | |
| | | oxy)phenyl)-4-(2,6-difluorobenzyl)- | | | |
| | | 2,4-dihydro-3H-1,2,4-triazol-3-one | | | |
| Example 6 Method 1 | +++ | 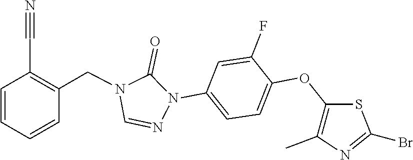 [SMILES: Cc1nc(Br)sc1Oc1ccc(-n2ncn(Cc3ccccc3C#N)c2=O)cc1F] | Yellow solid, yield: 18.8% | ¹H NMR (400 MHz, DMSO-d₆) δ 8.38 (s, 1H), 7.93 (dd, J = 12.4, 2.4 Hz, 1H), 7.74 (ddd, J = 9.2, 2.4, 1.2 Hz, 1H), 7.55-7.45 (m, 3H), 7.16 (t, J = 8.0 Hz, 2H), 4.98 (s, 2H), 2.35 (s, 3H). | 485.4, 487.4 |
| | | 2-((1-(4-((2-bromo-4-methylthiazol- | | | |
| | | 5-yl)oxy)-3-fluorophenyl)-5-oxo-1- | | | |
| | | 5-dihydro-4H-1,2,4-triazol-4-yl)meth- | | | |
| | | yl)benzonitrile | | | |
| Example 7 Method 1 | ++++ | 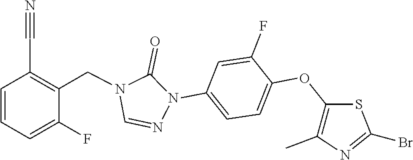 [SMILES: Cc1nc(Br)sc1Oc1ccc(-n2ncn(Cc3c(F)cccc3C#N)c2=O)cc1F] | Yellow solid, yield: 19.4% | ¹H NMR (400 MHz, DMSO-d₆) δ 8.40 (s, 1H), 7.91 (dd, J = 12.4, 2.4 Hz, 1H), 7.72 (ddd, J = 9.2, 2.4, 1.2 Hz, 1H), 7.50-7.44 (m, 2H), 7.16 (t, J = 8.0 Hz, 2H), 4.98 (s, 2H), 2.35 (s, 3H). | 503.9, 505.9 |
| | | 2-((1-(4-((2-bromo-4-methylthiazol- | | | |
| | | 5-yl)oxy)-3-fluorophenyl)-5-oxo-1,5- | | | |
| | | dihydro-4H-1,2,4-triazol-4-yl)meth- | | | |
| | | yl)-3-fluorobenzonitrile | | | |
| Example 8 Method 1 | ++++ | 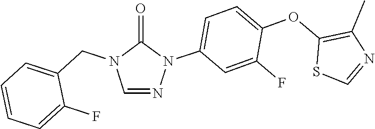 [SMILES: Cc1ncsc1Oc1ccc(-n2ncn(Cc3ccccc3F)c2=O)cc1F] | light- yellow solid, yield: 42.0% | ¹H NMR (400 MHz, DMSO-d₆) δ 8.73 (s, 1H), 8.36 (s, 1H), 7.90 (dd, J = 12.6, 2.5 Hz, 1H), 7.71 (ddd, J = 9.0, 2.6, 1.5 Hz, 1H), 7.40 (ddt, J = 11.8, 6.3, 3.7 Hz, 2H), 7.31-7.16 (m, 3H), 4.96 (s, 2H), 2.26 (s, 3H). | 401.1 |
| | | 2-(3-fluoro-4-((4-methylthiazol-5-yl) | | | |
| | | oxy)phenyl)-4-(2-fluorobenzyl)-2,4- | | | |
| | | dihydro-3H-1,2,4-triazol-3-one | | | |
| Example 9 Method 1 | +++ | 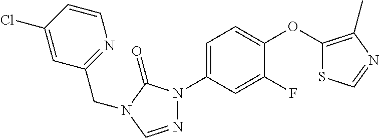 [SMILES: Cc1ncsc1Oc1ccc(-n2ncn(Cc3cc(Cl)ccn3)c2=O)cc1F] | yellow solid, yield: 37.6% | ¹H NMR (400 MHz, DMSO-d₆) δ 8.73 (s, 1H), 8.51 (d, J = 5.4 Hz, 1H), 8.37 (s, 1H), 7.91 (dd, J = 12.6, 2.5 Hz, 1H), 7.72 (ddd, J = 9.1, 2.6, 1.4 Hz, 1H), 7.63 (d, J = 2.0 Hz, 1H), 7.50 (dd, J = 5.4, 2.0 Hz, 1H), 7.25 (t, J = 9.1 Hz, 1H), 5.04 (s, 2H), 2.26 (s, 3H). | 418.1 |
| | | 4-((4-chloropyridin-2-yl)methyl)-2- | | | |
| | | (3-fluoro-4-((4-methylthiazol-5-yl) | | | |
| | | oxy)phenyl)-2,4-dihydro-3H-1,2,4-tri- | | | |
| | | azol-3-one | | | |

### Example 10: Method 2

### methyl 5-(4-(4-((4-chloropyridin-2-yl)methyl)-5-oxo-4,5-dihydro-1H-1,2,4-triazol-1-yl)-2-fluorophenoxy)-4-methylthiazole-2-carboxylate

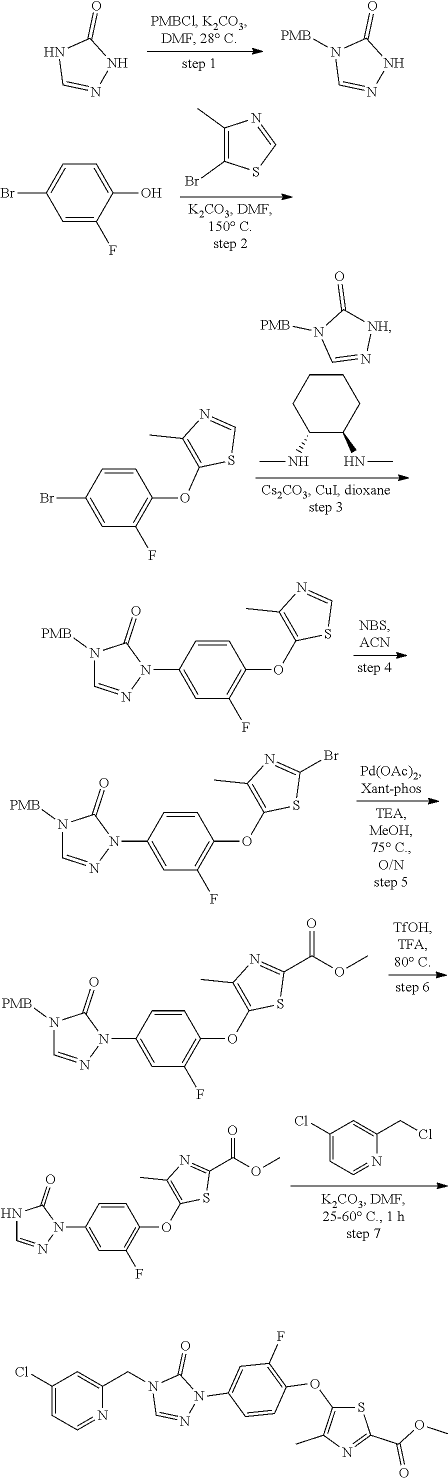

### Step 1: 4-(4-methoxybenzyl)-2,4-dihydro-3H-1,2,4-triazol-3-one

To a solution of 2,4-dihydro-3H-1,2,4-triazol-3-one (85 g, 1 mol, 1.0 eq.), and K₂CO₃ (207 g, 1.5 mol, 1.5 eq.) in DMF (800 mL), a solution of PMBCl (172 g, 1.1 mol, 1.1 eq.) in DMF (200 mL) was added dropwise slowly at 0° C. The resulted solution was stirred at 28° C. overnight. It was filtered and the filtrate was concentrated under vacuo and diluted with EA (500 mL), washed with brine (50 mL×2), dried over Na₂SO₄, concentrated to 200 mL, added PE (400 mL) and filtered to obtain a solid. 4-(4-methoxybenzyl)-2,4-dihydro-3H-1,2,4-triazol-3-one was obtained as a white solid (90 g, yield: 90%). LC-MS (m/z): 206.1 [M+H]⁺.

### Step 2: 5-(4-bromo-2-fluorophenoxy)-4-methylthiazole

A solution of 4-bromo-2-fluorophenol (54 g, 281 mmol), 5-bromo-4-methyl-1,3-thiazole (50 g, 281 mmol), K₂CO₃ (78 g, 562 mmol) in DMF (800 mL) was stirred at 150° C. for 2 h. The resulted solution was filtered and the filtrate was diluted with EA (1.5 L), washed with water (500 mL×2) and brine (1 L), dried over Na₂SO₄, concentrated and purified by silica gel column (EA: PE=0:1˜1:1) to obtain 5-(4-bromo-2-fluorophenoxy)-4-methylthiazole as an off-white solid (77 g, yield: 85%). LC-MS (m/z): 287.9 [M+H]⁺.

### Step 3: 2-(3-fluoro-4-((4-methylthiazol-5-yl)oxy)phenyl)-4-(4-methoxybenzyl)-2,4-dihydro-3H-1,2,4-triazol-3-one

A solution of 4-(2-methoxy-5-methylphenyl)-2H-1,2,4-triazol-3-one (55 g, 267 mmol), 5-(4-bromo-2-fluorophenoxy)-4-methyl-1,3-thiazole (77 g, 267 mmol), (1R,2R)-1-N,2-N-dimethylcyclohexane-1,2-diamine (11.4 g, 80 mmol), CuI (15 g, 80 mmol), Cs₂CO₃ (174 g, 534 mmol) in DMF (1 L) was stirred at 100° C. for 16 h. The resulted solution was filtered and the filtrate concentrated, purified by silica gel column (EA: PE=0:1˜2:1) to obtain 2-(3-fluoro-4-((4-methylthiazol-5-yl)oxy)phenyl)-4-(4-methoxybenzyl)-2,4-dihydro-3H-1,2,4-t riazol-3-one as a colorless oil (65 g, yield: 78%). LC-MS (m/z): 413.1 [M+H]⁺.

### Step 4: 2-(4-((2-bromo-4-methylthiazol-5-yl)oxy)-3-fluorophenyl)-4-(4-methoxybenzyl)-2,4-dihydro-3H-1,2,4-triazol-3-one

To a solution of 2-{3-fluoro-4-[(4-methyl-1,3-thiazol-5-yl)oxy]phenyl}-4-(2-methoxy-5-methylphenyl)-1,2,4-triazol-3-one (60 g, 146 mmol) in ACN (800 mL), a solution of NBS (31 g, 175 mmol) in ACN (200 mL) was added slowly at 0° C., the reaction was stirred at r.t. for 10 min. The resulted solution was filtered, and the filtrate was concentrated and purified by silica gel column with EA: PE=0:1˜2:1 to obtain 2-(4-((2-bromo-4-methylthiazol-5-yl)oxy)-3-fluorophenyl)-4-(4-methoxybenzyl)-2,4-dihydro-3H-1,2,4-triazol-3-one as a yellow solid (60 g, yield: 75%). LC-MS (m/z): 491.0 [M+H]⁺. ¹H NMR (400 MHz, CDCl₃) δ 7.92 (dd, J=12.2, 2.5 Hz, 1H), 7.79-7.72 (m, 1H), 7.41 (s, 1H), 7.29 (d, J=2.9 Hz, 1H), 7.27 (s, 1H), 7.04 (t, J=8.9 Hz, 1H), 6.94-6.89 (m, 2H), 4.79 (s, 2H), 3.81 (s, 3H), 2.30 (s, 3H).

### Step 5: methyl 5-(2-fluoro-4-(4-(4-methoxybenzyl)-5-oxo-4,5-dihydro-1H-1,2,4-triazol-1-yl) phenoxy)-4-methylthiazole-2-carboxylate

A solution of 2-{4-[(2-bromo-4-methyl-1,3-thiazol-5-yl)oxy]-3-fluorophenyl}-4-(2-methoxy-5-methylphenyl)-1,2,4-triazol-3-one (1.2 g, 2.4 mmol), Pd(OAc)₂ (110 mg, 0.4 mmol), Xant-Phos (280 mg, 0.4 mmol), TEA (1.2 g, 12 mmol) in MeOH (30 mL) was stirred under an atmosphere of CO at 75° C. for 16 h. The resulted solution was concentrated and purified by Combi-Flash (EA: PE=0:1˜2:1) to obtain methyl 5-(2-fluoro-4-(4-(4-methoxybenzyl)-5-oxo-4,5-dihydro-1H-1,2,4-triazol-1-yl)phenoxy)-4-meth ylthiazole-2-carboxylate as an off-white solid (600 mg, yield: 48%). LC-MS (m/z): 471.0 [M+H]⁺.

### Step 6: methyl 5-(2-fluoro-4-(5-oxo-4,5-dihydro-1H-1,2,4-triazol-1-yl)phenoxy)-4-methylthiazole-2-carboxylate

A solution of methyl 5-{2-fluoro-4-[4-(2-methoxy-5-methylphenyl)-5-oxo-1,2,4-triazol-1-yl]phenoxy}-4-methyl-1,3-thiazole-2-carboxylate (600 mg, 1.3 mmol), TfOH (574 mg, 3.8 mmol) in TFA (3 mL) was stirred at 80° C. for 16 h. The resulted solution was diluted with EA (30 mL), washed with water (30 mL), brine (30 mL), dried over Na₂SO₄, concentrated and purified by Combi-Flash (EA PE=0:1˜2:1) to obtain methyl 5-(2-fluoro-4-(5-oxo-4,5-dihydro-1H-1,2,4-triazol-1-yl)phenoxy)-4-methylthiazole-2-carboxylate (350 mg, yield: 70%) as a green solid. LC-MS (m/z): 413.0 [M+H]⁺.

### Step 7: methyl 5-(4-(4-((4-chloropyridin-2-yl)methyl)-5-oxo-4,5-dihydro-1H-1,2,4-triazol-1-yl)-2-fluorophenoxy)-4-methylthiazole-2-carboxylate

To a solution of 4-chloro-2-(chloromethyl)pyridine (30 mg, 0.185 mmol) in DMF (5 mL) was added methyl 5-[2-fluoro-4-(5-oxo-4H-1,2,4-triazol-1-yl)phenoxy]-4-methyl-thiazole-2-carboxylate (32 mg, 0.0926 mmol) and potassium carbonate (38 mg, 0.278 mmol). The reaction mixture was stirred at 25° C. for 2 h. The reaction was taken up in EtOAc (100 mL) and the organic layers were combined and washed with water (2×50 mL) then brine (50 mL). The reaction mixture was dried with MgSO₄ and evaporated to dryness. The crude product was then purified by Pre-TLC (PE:EA=2:3) to give methyl 5-[4-[4-[(4-chloro-2-pyridyl)methyl]-5-oxo-1,2,4-triazol-1-yl]-2-fluoro-phenoxy]-4-methyl-thi azole-2-carboxylate as white solid(25 mg, yield: 28.4%). MS (m/z): 476.1 [M+H]⁺. ¹H NMR (400 MHz, DMSO) δ 8.51 (d, J=5.4 Hz, 1H), 8.40 (s, 1H), 7.96 (dd, J=12.5, 2.5 Hz, 1H), 7.78 (ddd, J=9.1, 2.5, 1.4 Hz, 1H), 7.64 (d, J=2.0 Hz, 1H), 7.54-7.42 (m, 2H), 5.05 (s, 2H), 3.87 (s, 3H), 2.35 (s, 3H).

### Examples (Compounds) 11-12 were Synthesized Using a Similar Method to that Used in Example 10

| Compound | Ac- | | | | |
|---|---|---|---|---|---|
| No. | tivi- | | Appearance | | MS (m/z) |
| Method | ty | Structure and Name | and Yield | ¹H NMR Data | [M + H]⁺ |
| Example 10 Method 2 | ++ | 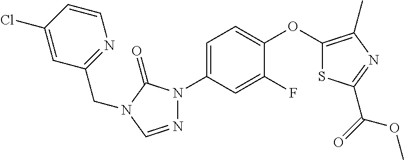 [SMILES: COC(=O)c1nc(C)c(Oc2ccc(-n3ncn(Cc4cc(Cl)ccn4)c3=O)cc2F)s1] | white solid, yield: 28.4% | ¹H NMR (400 MHz, DMSO-d₆) δ 8.51 (d, J = 5.4 Hz, 1H), 8.40 (s, 1H), 7.96 (dd, J = 12.5, 2.5 Hz, 1H), 7.78 (ddd, J = 9.1, 2.5, 1.4 Hz, 1H), 7.64 (d, J = 2.0 Hz, 1H), 7.54-7.42 (m, 2H), 5.05 (s, 2H), 3.87 (s, 3H), 2.35 (s, 3H). | 476.1 |
| | | methyl | | | |
| | | 5-(4-(4-((4-chloropyridin-2-yl) | | | |
| | | methyl)-5-oxo-4,5-dihydro-1H- | | | |
| | | 1,2,4- | | | |
| | | triazol-1-yl)-2-fluorophenoxy)- | | | |
| | | 4-methylthiazole-2-carboxylate | | | |
| Example 11 Method 2 | + | 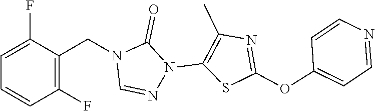 [SMILES: Cc1nc(Oc2ccncc2)sc1-n1ncn(Cc2c(F)cccc2F)c1=O] | white solid, yield: 7.1% | ¹H NMR (400 MHz, Chloroform-d) δ 8.07 (d, J = 7.6 Hz, 2H), 7.62 (s, 1H), 7.46-7.34 (m, 1H), 7.01 (t, J = 8.0 Hz, 2H), 6.56 (d, J = 7.2 Hz, 2H), 4.98 (s, 2H), 2.41 (s, 3H). | 402.2 |
| | | 4-(2,6-difluorobenzyl)-2-(4-meth- | | | |
| | | yl-2-(pyridin-4-yloxy)thiazol-5-yl)- | | | |
| | | 2,4-dihydro-3H-1,2,4-triazol-3- | | | |
| | | one | | | |
| Example 12 Method 2 | ++++ | 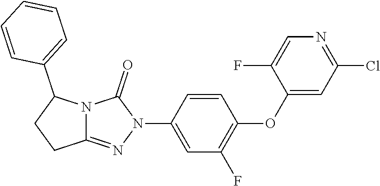 [SMILES: O=c1n(-c2ccc(Oc3cc(Cl)ncc3F)c(F)c2)nc2n1C(c1ccccc1)CC2] | grey solid, yield: 54.2% | ¹H NMR (400 MHz, Chloroform-d) δ 8.24 (d, J = 2.4 Hz, 1H), 8.04-7.96 (m, 1H), 7.87 (ddd, J = 9.2, 2.4, 1.6 Hz, 1H), 7.44-7.33 (m, 3H), 7.28- 7.21 (m, 3H), 6.65 (dd, J = 6.0, 1.2 Hz, 1H), 5.30 (dd, J = 7.6, 4.0 Hz, 1H), 3.16-3.00 (m, 2H), 3.00-2.89 (m, 1H), 2.64-2.48 (m, 1H). | 441.2 |
| | | 2-(4-((2-chloro-5-fluoropyridin-4- | | | |
| | | yl)oxy)-3-fluorophenyl)-5-phenyl- | | | |
| | | 2,5,6,7-tetrahydro-3H-pyrrolo[2,1- | | | |
| | | c][1,2,4]triazol-3-one | | | |

### Example 13: Method 3

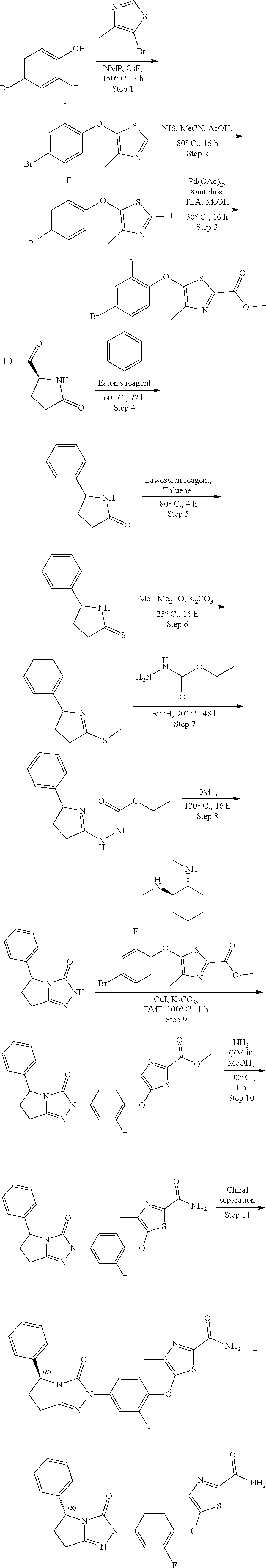

### Step 1: 5-(4-bromo-2-fluoro-phenoxy)-4-methyl-thiazole

To a solution of 4-Bromo-2-fluorophenal (1.00 eq, 20.0 g, 105 mmol) in NMP (200 mL), was added 5-Bromo-4-methylthiazole (1.10 eq, 20.5 g, 115 mmol) and CsF (3.00 eq, 49.9 g, 314 mmol), the mixture was stirred for 3 h at 150° C. under nitrogen atmosphere. The reaction was diluted with EtOAc (1000 mL) and the organics washed with water (500 mL×2), then saturated brine (500 mL). The organics were then separated and dried (with MgSO₄) before concentration to dryness. The residue was purified by silica gel chromatography eluted with 50% EtOAc in petroleum ether to give 5-(4-bromo-2-fluoro-phenoxy)-4-methyl-thiazole (18 g, 56.2 mmol, 53.7% yield) as a yellow oil. MS (m/z): 288.2, 290.2 [M+H]⁺.

### Step 2: 5-(4-bromo-2-fluoro-phenoxy)-2-iodo-4-methyl-thiazole

To a solution of 5-(4-bromo-2-fluoro-phenoxy)-4-methyl-thiazole (1.00 eq, 14 g, 48.6 mmol) in MeCN (300 mL), was added NIS (2.20 eq, 18.6 g, 106.8 mmol), AcOH (30.0 mL), the mixture was stirred for 16 h at 80° C. under nitrogen atmosphere. The reaction was quenched with saturated solution of Na₂S₂O₄. The mixture was diluted with EtOAc (500 mL) and the organics washed with water (200 mL×2), then saturated brine (200 mL). The organics were then separated and dried (MgSO₄) before concentration to dryness. The residue was purified by silica gel chromatography eluted with 30% EtOAc in petroleum ether to give 5-(4-bromo-2-fluoro-phenoxy)-2-iodo-4-methyl-thiazole (12 g, 29.0 mmol, 59.6% yield) as a yellow solid. MS (m/z): 414.2, 416.2 [M+H]⁺.

### Step 3: methyl 5-(4-bromo-2-fluoro-phenoxy)-4-methyl-thiazole-2-carboxylate

To a solution of 5-(4-bromo-2-fluoro-phenoxy)-2-iodo-4-methyl-thiazole (1.00 eq, 10 g, 24.2 mmol) in methanol (300 mL), was added Pd(OAc)₂ (0.30 eq, 1.62 g, 7.24 mmol), Xantphos (0.60 eq, 8.42 g, 14.50 mmol) and TEA (5.0 mL), the mixture was stirred for 16 h at 50° C. under CO balloon. The reaction was taken up in EtOAc (500 mL) and the organics washed with water (300 mL×2) and saturated brine (200 mL). The organics were then separated and dried (MgSO₄) before concentration to dryness. The crude was then purified by flash column chromatography eluted with 30% EtOAc in petroleum ether. The desired fractions were concentrated to dryness in vacuo to afford methyl 5-(4-bromo-2-fluoro-phenoxy)-4-methyl-thiazole-2-carboxylate (5.8 g, 15.1 mmol, 62.43% yield) as a brown oil. MS (m/z): 346.2, 348.2 [M+H]⁺. ¹H NMR (400 MHz, Chloroform-d) δ 7.38 (ddd, J=10.0, 2.4, 0.8 Hz, 1H), 7.27-7.23 (m, 1H), 6.94 (t, J=8.4 Hz, 1H), 3.98 (d, J=0.8 Hz, 3H), 2.41 (d, J=0.8 Hz, 3H).

### Step 4: 5-phenylpyrrolidin-2-one

A solution of benzene (242 g, 3.10 mol, 2.0 eq) in Eaton's reagent (500 mL) was added into a suspension of (S)-5-oxopyrrolidine-2-carboxylic acid (200 g, 1.55 mol, 1.0 eq) in Eaton's reagent (1500 mL). The reaction was heated to 60° C. for 72 h under nitrogen atmosphere. The mixture reaction was poured into ice water and adjusted to pH>7 by a NaOH aqueous solution. The aqueous layer was extracted with EtOAc, washed with water and brine, dried over Na₂SO₄, filtered, and concentrated to give 5-phenylpyrrolidin-2-one (130 g, 52% yield) as a white solid. MS (m/z): 162.2 [M+H]⁺.

### Step 5: 5-phenylpyrrolidine-2-thione

To a solution of 5-phenylpyrrolidin-2-one (130 g, 0.80 mol, 1.0 eq) in toluene (1200 mL) was added Lawession reagent (640.3 g, 1.59 mol, 2.0 eq). The mixture was stirred at 80° C. for 4 h under nitrogen atmosphere. Then cooled to 20° C. and concentrated, purified by chromatography (EtOAc/petroleum ether=1/6 to 1:1) to give 5-phenylpyrrolidine-2-thione (128 g, 91% yield) as a white solid. MS (m/z): 178.0 [M+H]⁺.

### Step 6: 5-(methylthio)-2-phenyl-3,4-dihydro-2H-pyrrole

To a solution of 5-phenylpyrrolidine-2-thione (128 g, 0.72 mol, 1.0 eq) in Me₂CO (1750 mL) was added MeI (122.5 g, 0.86 mol, 1.2 eq) and K₂CO₃ (249.7 g, 1.80 mol, 2.5 eq). The reaction mixture was stirred at 25° C. for 16 h under nitrogen atmosphere. The mixture reaction was extracted with EtOAc, and the organic layer was washed with water and brine, then dried over Na₂SO₄, filtered and concentrated. The residue was purified by chromatography (EtOAc/petroleum ether=2/1) to give 5-(methylthio)-2-phenyl-3,4-dihydro-2H-pyrrole (77 g, 55.7% yield) as a white oil. MS (m/z): 192.1 [M+H]⁺.

### Step 7: ethyl 2-(2-phenyl-3,4-dihydro-2H-pyrrol-5-yl)hydrazine-1-carboxylate

To a solution of 5-(methylthio)-2-phenyl-3,4-dihydro-2H-pyrrole (77 g, 0.40 mol, 1.0 eq) in EtOH (500 mL) was added ethyl hydrazinecarboxylate (41.9 g, 0.40 mol, 1.0 eq), the mixture reaction was heated to 90° C. for 48 h under nitrogen atmosphere. The reaction mixture was concentrated and the residue was washed with EtOAc/petroleum ether (5/1). The solid was collected to give ethyl 2-(2-phenyl-3,4-dihydro-2H-pyrrol-5-yl)hydrazine-1-carboxylate (80 g, 80.0% yield) as a white solid. MS (m/z): 248.2 [M+H]⁺.

### Step 8:5-phenyl-2,5,6,7-tetrahydro-3H-pyrrolo[2,1-c][1,2,4]triazol-3-one

Ethyl 2-(2-phenyl-3,4-dihydro-2H-pyrrol-5-yl)hydrazine-1-carboxylate (40 g, 0.16 mol) in DMF (200 mL) was stirred at 130° C. for 16 h. DMF was evaporated in vacuum. The residue was purified by chromatography (MeOH/DCM=2% to 10%) to provide 5-phenyl-2,5,6,7-tetrahydro-3H-pyrrolo[2,1-c][1,2,4]triazol-3-one (16 g, 49.0% yield) as a white solid. MS (m/z): 202.1 [M+H]⁺. ¹H NMR (400 MHz, Chloroform-d) δ 9.35 (brs, 1H), 7.41-7.29 (m, 3H), 7.20 (d, J=7.2 Hz, 2H), 5.22 (dd, J=8.0, 4.0 Hz, 1H), 3.08-2.98 (m, 1H), 2.97-2.86 (m, 1H), 2.86-2.75 (m, 1H), 2.51-2.39 (m, 1H).

### Step 9: methyl 5-[2-fluoro-4-(3-oxo-5-phenyl-6,7-dihydro-5H-pyrrolo[2,1-c][1,2,4]triazol-2-yl)phenoxy]-4-methyl-thiazole-2-carboxylate

To a solution of 5-phenyl-2,5,6,7-tetrahydropyrrolo[2,1-c][1,2,4]triazol-3-one (1.00 eq, 2906 mg, 14.4 mmol) in DMF (250 mL), was added CuI (0.50 eq, 1379 mg, 7.22 mmol), K₂CO₃ (2.00 eq, 3986 mg, 28.9 mmol), methyl 5-(4-bromo-2-fluoro-phenoxy)-4-methyl-thiazole-2-carboxylate (1.00 eq, 5000 mg, 14.4 mmol), (1R,2R)—N1,N2-dimethylcyclohexane-1,2-diamine (1.00 eq, 2051 mg, 14.4 mmol). The reaction mixture was stirred for 1 h at 100° C. under nitrogen. The reaction was concentrated to dryness and the residue was taken up in EtOAc (1000 mL) and the organics washed with water (300 mL×2) and saturated brine (300 mL). The organics were then separated and dried (with MgSO₄) before concentration to dryness. The crude was then purified by flash column chromatography eluted with 50% EtOAc in petroleum ether. The desired fractions were concentrated to dryness in vacuo to afford methyl 5-[2-fluoro-4-(3-oxo-5-phenyl-6,7-dihydro-5H-pyrrolo[2,1-c][1,2,4]triazol-2-yl)phenoxy]-4-m ethyl-thiazole-2-carboxylate (5 g, 10.2 mmol, 70.5% yield) as a yellow oil. MS (m/z): 467.2 [M+H]⁺.

methyl 5-[2-fluoro-4-(3-oxo-5-phenyl-6,7-dihydro-5H-pyrrolo[2,1-c][1,2,4]triazol-2-yl) phenoxy]-4-methyl-thiazole-2-carboxylate (10.0 g) was purified by chiral separation by SFC under condition (column: Lux Cellulose-4; column Size: 5 cm×25 cm, Sum; Mobile Phase A: CO₂/Mobile Phase B: MeOH:CAN; Wave Length: UV 220 nm; 25° C.).

### methyl (R)-5-(2-fluoro-4-(3-oxo-5-phenyl-6,7-dihydro-3H-pyrrolo[2,1-c][1,2,4]triazol-2(5H)-yl)phenoxy)-4-methylthiazole-2-carboxylate

4.545 g white solid, the first peak (Peak-1: ee %=99.86%, chemical purity=96.97%, retention time: 1.356 min), MS (m/z): 467.2 [M+H]⁺. H NMR (400 MHz, Chloroform-d)¹H NMR (400 MHz, Chloroform-d) δ 7.93 (dd, J=12.4, 2.4 Hz, 1H), 7.76 (dt, J=9.2, 2.0 Hz, 1H), 7.44-7.32 (m, 3H), 7.27-7.23 (m, 2H), 7.11 (t, J=8.8 Hz, 1H), 5.32-5.25 (m, 1H), 3.96 (s, 3H), 3.13-2.99 (m, 2H), 2.99-2.87 (m, 1H), 2.59-2.45 (m, 1H), 2.43 (s, 3H).

### methyl (S)-5-(2-fluoro-4-(3-oxo-5-phenyl-6,7-dihydro-3H-pyrrolo[2,1-c][1,2,4]triazol-2(5H)-yl)phenoxy)-4-methylthiazole-2-carboxylate

4.575 g white solid, the second peak (Peak-2: ee %=99.92%, chemical purity=99.50%, retention time: 1.766 min), MS (m/z): 467.2 [M+H]⁺. δ 7.93 (dd, J=12.4, 2.4 Hz, 1H), 7.76 (dt, J=9.2, 2.0 Hz, 1H), 7.44-7.32 (m, 3H), 7.26-7.22 (m, 2H), 7.11 (t, J=8.8 Hz, 1H), 5.28 (dd, J=7.6, 4.0 Hz, 1H), 3.96 (s, 3H), 3.18-2.99 (m, 2H), 2.97-2.91 (m, 1H), 2.59-2.47 (m, 1H), 2.43 (s, 3H).

### Step 10: 5-(2-fluoro-4-(3-oxo-5-phenyl-6,7-dihydro-3H-pyrrolo[2,1-c][1,2,4]triazol-2(5H)-yl)phenoxy)-4-methylthiazole-2-carboxamide

To a solution of methyl 5-[2-fluoro-4-(3-oxo-5-phenyl-6,7-dihydro-5H-pyrrolo [2,1-c][1,2,4]triazol-2-yl)phenoxy]-4-methyl-thiazole-2-carboxylate (1.00 eq, 5 g, 10.7 mmol) in methanol (5 mL), was added NH₃ (7M in MeOH) (30 mL). The reaction mixture was stirred for 1 h at 100° C. under nitrogen. The reaction mixture was concentrated to dryness, then triturated with MeOH to give 5-(2-fluoro-4-(3-oxo-5-phenyl-6,7-dihydro-3H-pyrrolo[2,1-c][1,2,4]triazol-2(5H)-yl)phenoxy)-4-methylthiazole-2-carboxami de (4 g, 8.8 mmol, 68.2% yield) as an off-white solid. MS (m/z): 452.2 [M+H]⁺. ¹H NMR (400 MHz, DMSO-d₆) δ 8.09 (brs, 1H), 7.88 (dd, J=12.8, 2.4 Hz, 1H), 7.79 (brs, 1H), 7.70 (dt, J=9.2, 2.0 Hz, 1H), 7.42-7.37 (m, 3H), 7.36-7.30 (m, 3H), 5.32 (dd, J=8.0, 4.8 Hz, 1H), 3.07-2.97 (m, 2H), 2.97-2.85 (m, 1H), 2.38-2.32 (m, 1H), 2.31 (s, 3H).

### Step 11

5-(2-fluoro-4-(3-oxo-5-phenyl-6,7-dihydro-3H-pyrrolo[2,1-c][1,2,4]triazol-2(5H)-yl)phenoxy)-4-methylthiazole-2-carboxamide(1.0 g) was separated by chiral separation by SFC under basic condition [column: Lux Cellulose-4; column Size: 5 cm×25 cm, Sum; Mobile Phase A: CO₂/Mobile Phase B: MeOH:CAN(0.5% 2 mM NH₃-MeOH); Wave Length: UV 220 nm; 25° C.)] to give two compounds.

### (R)-5-(2-fluoro-4-(3-oxo-5-phenyl-6,7-dihydro-3H-pyrrolo[2,1-c][1,2,4]triazol-2(5H)-yl)ph enoxy)-4-methylthiazole-2-carboxamide

0.403 g white solid, yield: 40.3%, the first peak (peak-1: ee %=97.50%, chemical purity=99.41%, retention time: 1.940 min), MS (m/z): 452.2 [M+H]⁺. ¹H NMR (400 MHz, Chloroform-d) δ 7.92 (dd, J=12.4, 2.4 Hz, 1H), 7.74 (dt, J=9.2, 2.0 Hz, 1H), 7.43-7.37 (m, 2H), 7.36-7.31 (m, 1H), 7.27-7.23 (m, 1H), 7.15-7.04 (m, 2H), 5.50 (brs, 1H), 5.28 (dd, J=7.6, 4.0 Hz, 1H), 3.13-2.98 (m, 2H), 2.98-2.86 (m, 1H), 2.61-2.46 (m, 1H), 2.37 (s, 3H).

### (S)-5-(2-fluoro-4-(3-oxo-5-phenyl-6,7-dihydro-3H-pyrrolo[2,1-c][1,2,4]triazol-2(5H)-yl)phenoxy)-4-methylthiazole-2-carboxamide

0.374 g white solid, yield: 37.4, the second peak (peak-2: ee %=98.50%, chemical purity=98.91%, retention time: 2.198 min), MS (m/z): 452.2 [M+H]³⁰. ¹H NMR (400 MHz, DMSO-d₆) δ 8.09 (brs, 1H), 7.88 (dd, J=12.8, 2.4 Hz, 1H), 7.79 (brs, 1H), 7.73-7.66 (m, 1H), 7.43-7.37 (m, 3H), 7.36-7.30 (m, 3H), 5.32 (dd, J=8.0, 4.8 Hz, 1H), 3.09-2.96 (m, 2H), 2.96-2.87 (m, 1H), 2.39-2.28 (n, 4H).

Examples (Compounds) 14-36, 685-702 were synthesized using a similar method to that used in Example 13

| Com - | | | Appear- | | MS |
|---|---|---|---|---|---|
| pound | | | ance | | (m/z) |
| No. | Activi- | | and | | [M + |
| Method | ty | Structure and Name | Yield | ¹H NMR Data | H]⁺ |
| Example 13 Method 3 | ++++ | 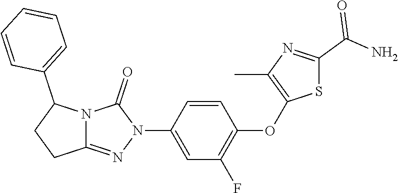 [SMILES: Cc1nc(C(N)=O)sc1Oc1ccc(-n2nc3n(c2=O)C(c2ccccc2)CC3)cc1F] | off-white solid, yield: 68.2% | ¹H NMR (400 MHz, DMSO-d₆) δ 8.09 (brs, 1H), 7.88 (dd, J = 12.8, 2.4 Hz, 1H), 7.79 (brs, 1H), 7.70 (dt, J = 9.2, 2.0 Hz, 1H), 7.42-7.37 (m, 3H), 7.36-7.30 (m, 3H), 5.32 (dd, J = 8.0, 4.8 Hz, 1H), 3.07-2.97 (m, 2H), 2.97-2.85 (m, 1H), 2.38-2.32 (m, 1H), 2.31 (s, 3H). | 452.2 |
| | | 5-(2-fluoro-4-(3-oxo-5-phenyl-6,7- | | | |
| | | dihydro-3H-pyrrolo[2,1-c][1,2,4] | | | |
| | | triazol-2(5H)-yl)phenoxy)-4-meth- | | | |
| | | ylthiazole-2-carboxamide | | | |
| Example 14 Method 3 Single unknown stereo- isomer | ++++ | 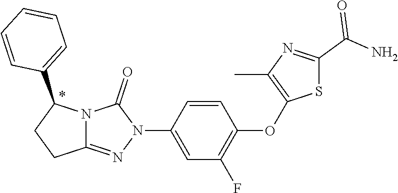 [SMILES: Cc1nc(C(N)=O)sc1Oc1ccc(-n2nc3n(c2=O)[C@H](c2ccccc2)CC3)cc1F] | white solid, yield: 43.5% | ¹H NMR (400 MHz, DMSO-d₆) δ 8.09 (brs, 1H), 7.88 (dd, J = 12.8, 2.4 Hz, 1H), 7.79 (brs, 1H), 7.73-7.66 (m, 1H), 7.43-7.37 (m, 3H), 7.36-7.30 (m, 3H), 5.32 (dd, J = 8.0, 4.8 Hz, 1H), 3.09-2.96 (m, 2H), 2.96-2.87 (m, 1H), 2.39-2.28 (m, 4H). | 452.3 |
| | | (S)-5-(2-fluoro-4-(3-oxo-5-phenyl- | | | |
| | | 6,7-dihydro-3H-pyrrolo[2,1-c][1,2,4,] | | | |
| | | triazol-2(5H)-yl)phenoxy)-4- | | | |
| | | methylthiazole-2-carboxamide | | | |
| Example 15 Method 3 Single unknown stereo- isomer | +++ | 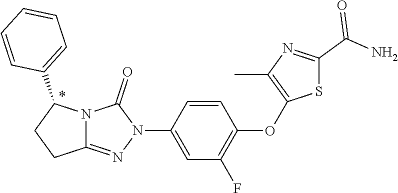 [SMILES: Cc1nc(C(N)=O)sc1Oc1ccc(-n2nc3n(c2=O)[C@@H](c2ccccc2)CC3)cc1F] | white solid, yield: 45.9% | ¹H NMR (400 MHz, Chloroform-d) δ 7.92 (dd, J = 12.4, 2.4 Hz, 1H), 7.74 (dt, J = 9.2, 2.0 Hz, 1H), 7.43-7.37 (m, 2H), 7.36-7.31 (m, 1H), 7.27- 7.23 (m, 1H), 7.15-7.04 (m, 2H), 5.50 (brs, 1H), 5.28 (dd, J = 7.6, 4.0 Hz, 1H), 3.13-2.98 (m, 2H), 2.98- 2.86 (m, 1H), 2.61-2.46 (m, 1H), 2.37 (s, 3H). | 452.2 |
| | | (R)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methylthiazole-2-carboxamide | | | |
| Example 16 Method 3 Single unknown stereo- isomer | ++++ | 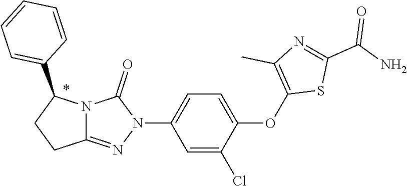 [SMILES: Cc1nc(C(N)=O)sc1Oc1ccc(-n2nc3n(c2=O)[C@H](c2ccccc2)CC3)cc1Cl] | white solid, yield: 9.8% | ¹H NMR (400 MHz, Chloroform-d) δ 8.15 (d, J = 2.4 Hz, 1H), 7.87 (dd, J = 9.2, 2.4 Hz, 1H), 7.37 (dq, J = 14.4, 7.2 Hz, 3H), 7.27-7.23 (m, 2H), 7.06-6.94 (m, 2H), 5.54 (s, 1H), 5.28 (dd, J = 7.6, 4.4 Hz, 1H), 3.05 (dt, J = 14.0, 8.0 Hz, 2H), 3.00-2.86 (m, 1H), 2.54 (td, J = 11.2, 10.0, 5.2 Hz, 1H), 2.34 (s, 3H). | 468.2 |
| | | (S)-5-(2-chloro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methylthiazole-2-carboxamide | | | |
| Example 17 Method 3 Single unknown stereo- isomer | ++++ | 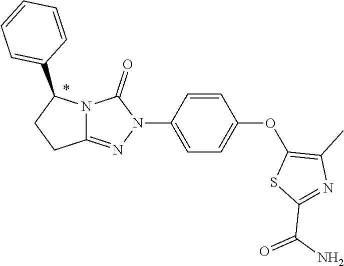 [SMILES: Cc1nc(C(N)=O)sc1Oc1ccc(-n2nc3n(c2=O)[C@H](c2ccccc2)CC3)cc1] | light yellow solid, yield: 11.7% | ¹H NMR (400 MHz, Chloroform-d) δ 7.94 (d, J = 8.8 Hz, 2H), 7.43-7.29 (m, 3H), 7.27-7.23 (m, 2H), 7.11- 7.03 (m, 3H), 5.49 (brs, 1H), 5.29 (dd, J = 7.6, 4.0 Hz, 1H), 3.11-2.98 (m, 2H), 2.98-2.86 (m, 1H), 2.57- 2.48 (m, 1H), 2.31 (s, 3H). | 434.2 |
| | | (S)-4-methyl-5-(4-(3-oxo-5-phenyl- | | | |
| | | 6,7-dihydro-3H-pyrrolo[2,1-c][1,2,4] | | | |
| | | triazol-2(5H)-yl)phenoxy)thi- | | | |
| | | azole-2-carboxamide | | | |
| Example 18 Method 3 Single unknown stereo- isomer | +++++ | 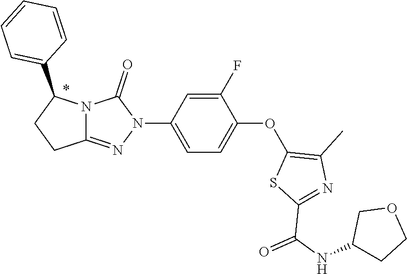 | white solid, yield: 41.1% | ¹H NMR (400 MHz, DMSO-d₆) δ 8.87 (d, J = 7.2 Hz, 1H), 7.88 (dd, J = 12.4, 2.4 Hz, 1H), 7.69 (d, J = 9.6 Hz, 1H), 7.43-7.29 (m, 6H), 5.32 (dd, J = 8.0, 4.8 Hz, 1H), 4.46-4.37 (m, 1H), 3.89-3.78 (m, 2H), 3.70 (q, J = 7.6 Hz, 1H), 3.58 (dd, J = 8.8, 5.2 Hz, 1H), 3.09-2.98 (m, 2H), 2.96-2.86 (m, 1H), 2.38-2.28 (m, 4H), 2.18- 2.05 (m, 1H), 2.05-1.93 (m, 1H). | 522.2 |
| | | 5-(2-fluoro-4-((S)-3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methyl-N-((S)-tetrahydrofuran-3- | | | |
| | | yl)thiazole-2-carboxamide | | | |
| Example 19 Method 3 Single unknown stereo- isomer | ++++ | 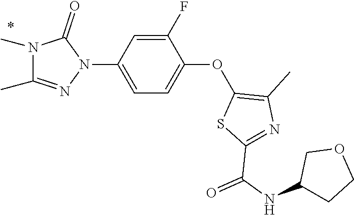 | white solid, yield: 39.5% | ¹H NMR (400 MHz, DMSO-d₆) δ 8.88 (d, J = 6.4 Hz, 1H), 7.88 (dd, J = 12.8, 2.4 Hz, 1H), 7.70 (d, J = 9.2 Hz, 1H), 7.44-7.29 (m, 6H), 5.32 (dd, J = 8.0, 4.8 Hz, 1H), 4.46-4.37 (m, 1H), 3.89-3.78 (m, 2H), 3.70 (q, J = 7.6 Hz, 1H), 3.58 (dd, J = 8.8, 4.8 Hz, 1H), 3.09-2.97 (m, 2H), 2.96-2.82 (m, 1H), 2.42-2.25 (m, 4H), 2.18- 2.04 (m, 1H), 2.05-1.93 (m, 1H). | 522.2 |
| | | 5-(2-fluoro-4-((S)-3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methyl-N-((S)-tetrahydrofuran-3- | | | |
| | | yl)thiazole-2-carboxamide | | | |
| Example | ++++ | (S)-2-(4-((2-((2S,6R)-2,6-dimethyl- | white | ¹H NMR (400 MHz, DMSO-d₆) δ | 550.2 |
| 20 | | morpholine-4-carbonyl)-4-methyl- | solid, | 7.88 (d, J = 13.2 Hz, 1H), 7.71 (d, J = | |
| Method 3 | | thiazol-5-yl)oxy)-3-fluorophenyl)- | yield: | 9.2 Hz, 1H), 7.45-7.36 (m, 3H), | |
| Single | | 5-phenyl-2,5,6,7-tetrahydro-3H- | 31.6% | 7.36-7.29 (m, 3H), 5.34-5.30 (m, | |
| unknown | | pyrrolo[2,1-c][1,2,4]triazol-3-one | | 2H), 4.35-4.18 (m, 1H), 3.70-3.48 | |
| stereo- | | | | (m, 3H), 3.10-2.83 (m, 3H), 2.40- | |
| isomer | | | | 2.25 (m, 5H), 1.12 (d, J = 6.2 Hz, | |
| | | | | 6H). | |
| Example | ++++ | (S)-5-(2-fluoro-4-(3-oxo-5-phenyl- | white | ¹H NMR (400 MHz, DMSO-d₆) δ | 508.2 |
| 21 | | 6,7-dihydro-3H-pyrrolo[2,1-c][1,2,4] | solid, | 9.54 (brs, 1H), 7.88 (dd, J = 12.8, 2.4 | |
| Method 3 | | triazol-2(5H)-yl)phenoxy)-4- | yield: | Hz, 1H), 7.73-7.66 (m, 1H), 7.44- | |
| Single | | methyl-N-(oxetan-3-yl)thiazole-2- | 29.6% | 7.29 (m, 6H), 5.32 (dd, J = 8.0, 4.8 | |
| unknown | | carboxamide | | Hz, 1H), 4.97 (p, J = 7.2 Hz, 1H), | |
| stereo- | | | | 4.73-4.62 (m, 4H), 3.09-2.85 (m, | |
| isomer | | | | 3H), 2.38-2.29 (m, 4H). | |
| Example 22 Method 3 | ++++ | 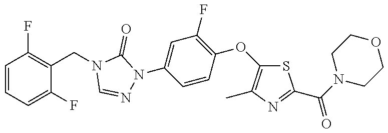 [SMILES: Cc1nc(C(=O)N2CCOCC2)sc1Oc1ccc(-n2ncn(Cc3c(F)cccc3F)c2=O)cc1F] | white solid, yield: 22.0% | ¹H NMR (400 MHz, DMSO-d₆) δ 8.36 (s, 1H), 7.90 (dd, J = 12.8, 2.4 Hz, 1H), 7.72 (dd, J = 9.2, 2.4 Hz, 1H), 7.46 (dq, J = 32.4, 9.2, 8.4 Hz, 2H), 7.16 (t, J = 8.0 Hz, 2H), 4.98 (s, 2H), 3.69-3.59 (m, 4H), 2.52-2.33 (m, 4H) 2.30 (s, 3H). | 532.1 |
| | | 4-(2,6-difluorobenzyl)-2-(3-fluoro- | | | |
| | | 4-((4-methyl-2-(morpholin-4-car- | | | |
| | | bonyl)thiazol-5-yl)oxy)phenyl)- | | | |
| | | 2,4-dihydro-3H-1,2,4-triazol-3-one | | | |
| Example 23 Method 3 | ++++ | 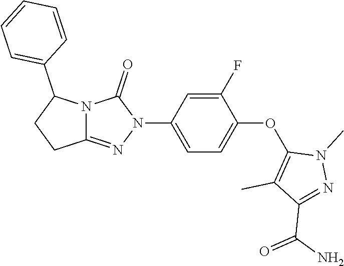 [SMILES: Cc1c(C(N)=O)nn(C)c1Oc1ccc(-n2nc3n(c2=O)C(c2ccccc2)CC3)cc1F] | white solid, yield: 18.2% | ¹H NMR (400 MHz, Chloroform-d) δ 7.91 (dd, J = 12.4, 2.4 Hz, 1H), 7.65 (dt, J = 9.2, 2.0 Hz, 1H), 7.37 (dq, J = 14.0, 7.2 Hz, 3H), 7.25-7.15 (m, 2H), 6.84-6.75 (m, 2H), 5.31-5.24 (m, 2H), 3.71 (s, 3H), 3.13-3.01 (m, 2H), 2.98-2.84 (m, 1H), 2.53 (tt, J = 9.6, 5.6 Hz, 1H), 2.09 (s, 3H). | 449.2 |
| | | 5-(2-fluoro-4-(3-oxo-5-phenyl-6,7- | | | |
| | | dihydro-3H-pyrrolo[2,1-c][1,2,4] | | | |
| | | triazol-2(5H)-yl)phenoxy)-1,4- | | | |
| | | dimethyl-1H-pyrazole-3-carboxa- | | | |
| | | mide | | | |
| Example 24 Method 3 | ++++ | 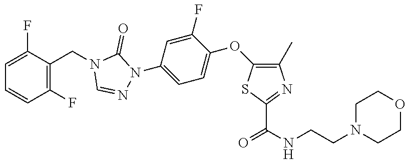 [SMILES: Cc1nc(C(=O)NCCN2CCOCC2)sc1Oc1ccc(-n2ncn(Cc3c(F)cccc3F)c2=O)cc1F] | white solid, yield: 53.8% | ¹H NMR (400 MHz, Chloroform-d) δ 7.93 (dd, J = 12.0, 2.4 Hz, 1H), 7.75 (dt, J = 9.2, 2.0 Hz, 1H), 7.58 (s, 1H), 7.47 (brs, 1H), 7.43-7.31 (m, 1H), 7.06 (t, J = 8.8 Hz, 1H), 6.98 (t, J = 8.0 Hz, 2H), 4.97 (s, 2H), 3.79-3.72 (m, 4H), 3.60-3.51 (m, 2H), 2.71- 2.46 (m, 6H), 2.35 (s, 3H). | 575.2 |
| | | 5-(4-(4-(2,6-diflurobenzyl)-5-oxo- | | | |
| | | 4,5-dihydro-1H-1,2,4-triazol-1- | | | |
| | | yl)-2-fluorophenoxy)-4-methyl-N- | | | |
| | | (2-morpholinoethyl)thiazole-2-car- | | | |
| | | boxamide | | | |
| Example 25 Method 3 | ++++ | 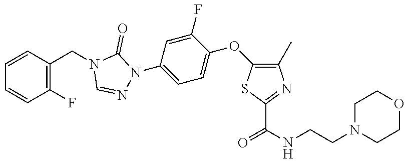 [SMILES: Cc1nc(C(=O)NCCN2CCOCC2)sc1Oc1ccc(-n2ncn(Cc3ccccc3F)c2=O)cc1F] | white solid, yield: 29.9% | ¹H NMR (400 MHz, Chloroform-d) δ 7.92 (dd, J = 12.0, 2.4 Hz, 1H), 7.75 (dt, J = 9.2, 2.0 Hz, 1H), 7.58 (s, 1H), 7.52-7.47 (m, 1H), 7.44 (td, J = 7.6, 1.6 Hz, 1H), 7.40-7.32 (m, 1H), 7.21-7.03 (m, 3H), 4.91 (d, J = 1.2 Hz, 2H), 3.75 (t, J = 4.8 Hz, 4H), 3.55 (q, J = 6.0 Hz, 2H), 2.61 (t, J = 6.0 Hz, 2H), 2.57-2.50 (m, 4H), 2.35 (s, 3H). | 557.2 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-4-methyl-N-(2- | | | |
| | | morpholinoethyl)thiazole-2-carbox- | | | |
| | | amide | | | |
| Example 26 Method 3 | +++ | 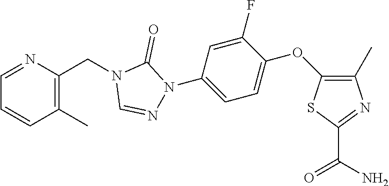 [SMILES: Cc1cccnc1Cn1cnn(-c2ccc(Oc3sc(C(N)=O)nc3C)c(F)c2)c1=O] | yellow solid, yield: 43.5% | ¹H NMR (400 MHz, DMSO-d₆) δ 8.33-8.28 (m, 2H), 8.10 (brs, 1H), 7.96 (dd, J = 12.8, 2.4 Hz, 1H), 7.81- 7.74 (m, 2H), 7.64 (d, J = 7.6 Hz, 1H), 7.41 (t, J = 9.2 Hz, 1H), 7.24 (dd, J = 7.6, 4.8 Hz, 1H), 5.03 (s, 2H), 2.38 (s, 3H), 2.31 (s, 3H). | 441.2 |
| | | 5-(2-fluoro-4-(4-((3-methylpyridin- | | | |
| | | 2-yl)methyl)-5-oxo-4,5-dihydro- | | | |
| | | 1H-1,2,4-triazol-1-yl)phenoxy)-4- | | | |
| | | methylthiazole-2-carboxamide | | | |
| Example 27 Method 3 | ++++ | 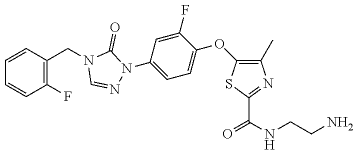 [SMILES: Cc1nc(C(=O)NCCN)sc1Oc1ccc(-n2ncn(Cc3ccccc3F)c2=O)cc1F] | white solid, yield: 66.3% | ¹H NMR (400 MHz, DMSO-d₆) δ 8.68 (brs, 1H), 8.37 (s, 1H), 7.93 (dd, J = 12.4, 2.4 Hz, 1H), 7.73 (dt, J = 9.2, 1.6 Hz, 1H), 7.39 (td, J = 9.2, 8.4, 3.2 Hz, 3H), 7.25-7.18 (m, 2H), 4.96 (s, 2H), 3.27-3.20 (m, 2H), 2.68 (t, J = 6.4 Hz, 2H), 2.31 (s, 3H), 1.23 (brs, 1H). | 487.3 |
| | | N-(2-aminoethyl)-5-(2-fluoro-4-(4- | | | |
| | | (2-fluorobenzyl)-5-oxo-4,5-dihy- | | | |
| | | dro-1H-1,2,4-triazol-1-yl)phenoxy)- | | | |
| | | 4-methylthiazole-2-carboxamide | | | |
| Example 28 Method 3 | ++++ | 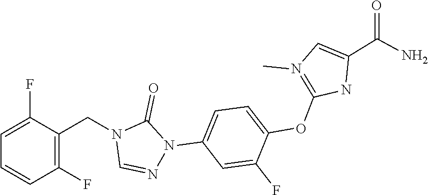 | white solid, yield: 37.2% | ¹H NMR (400 MHz, DMSO-d₆) δ 8.36 (s, 1H), 7.87 (dd, J = 12.4, 2.4 Hz, 1H), 7.75-7.60 (m, 2H), 7.52- 7.42 (m, 2H), 7.16 (t, J = 8.1 Hz, 2H), 6.95 (s, 1H), 4.99 (s, 2H), 3.59 (s, 3H). | 445.0 |
| | | 2-(4-(4-(2,6-difluorobenzyl)-5-oxo- | | | |
| | | 4,5-dihydro-1H-1,2,4-triazol-1- | | | |
| | | yl)-2-fluorophenoxy)-1-methyl-1H- | | | |
| | | imidazole-4-carboxamide | | | |
| Example 29 Method 3 | ++++ | 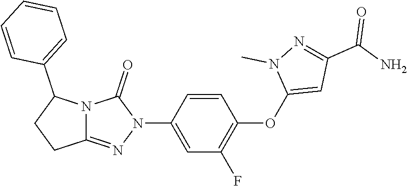 [SMILES: Cn1nc(C(N)=O)cc1Oc1ccc(-n2nc3n(c2=O)C(c2ccccc2)CC3)cc1F] | white solid, yield: 56.4% | ¹H NMR (400 MHz, CDCl₃) δ 7.96- 7.88 (m, 1H), 7.77 (d, J = 1.2 Hz, 1H), 7.44-7.33 (m, 3H), 7.27 (s, 1H), 7.25 (d, J = 2.8 Hz, 1H), 7.21 (d, J = 8.8 Hz, 1H), 6.06 (s, 1H), 5.31- 5.28 (m, 1H), 3.83 (s, 3H), 3.13- 2.85 (m, 3H), 2.58-2.45 (m, 1H). | 435.4 |
| | | 5-(2-fluoro-4-(3-oxo-5-phenyl-6,7- | | | |
| | | dihydro-3H-pyrrolo[2,1-c][1,2,4] | | | |
| | | triazol-2(5H)-yl)phenoxy)-1-meth- | | | |
| | | yl-1H-pyrazole-3-carboxamide | | | |
| Example 30 Method 3 Single unknown stereo- isomer | ++++ | 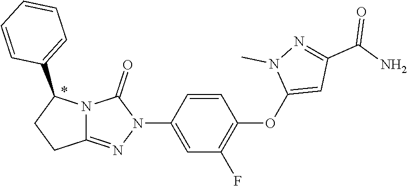 [SMILES: Cn1nc(C(N)=O)cc1Oc1ccc(-n2nc3n(c2=O)[C@H](c2ccccc2)CC3)cc1F] | white solid, yield: 74.6% | ¹H NMR (400 MHz, DMSO-d₆) δ 7.96-7.88 (m, 1H), 7.77 (d, J = 1.2 Hz, 1H), 7.44-7.33 (m, 3H), 7.26 (s, 1H), 7.24 (d, J = 2.8 Hz, 1H), 7.20 (d, J = 8.8 Hz, 1H), 6.05 (s, 1H), 5.36- 5.28 (m, 1H), 3.78 (s, 3H), 3.12- 2.86 (m, 3H), 2.58-2.45 (m, 1H). | 435.1 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-1- | | | |
| | | methyl-1H-pyrazole-3-carboxamide | | | |
| Example 31 Method 3 | ++++ | 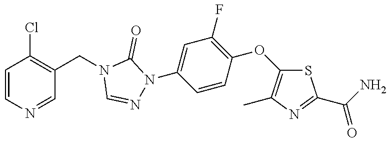 [SMILES: Cc1nc(C(N)=O)sc1Oc1ccc(-n2ncn(Cc3cnccc3Cl)c2=O)cc1F] | white solid, yield: 16.7% | ¹H NMR (400 MHz, DMSO-d₆) δ 8.63 (s, 1H), 8.54 (d, J = 5.2 Hz, 1H), 8.38 (s, 1H), 8.09 (s, 1H), 7.92 (dd, J = 12.4, 2.4 Hz, 1H), 7.80 (s, 1H), 7.73 (dt, J = 8.8, 2.0 Hz, 1H), 7.62 (d, J = 5.2 Hz, 1H), 7.39 (t, J = 9.2 Hz, 1H), 5.04 (s, 2H), 2.30 (s, 3H). | 461.9 |
| | | 5-(4-(4-((4-chloropyridin-3-yl)meth- | | | |
| | | yl)-5-oxo-4,5-dihydro-1H-1,2,4- | | | |
| | | triazol-1-yl)-2-fluorophenoxy)-4- | | | |
| | | methylthiazole-2-carboxamide | | | |
| Example 32 Method 3 | +++ | 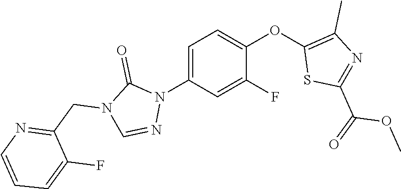 [SMILES: COC(=O)c1nc(C)c(Oc2ccc(-n3ncn(Cc4ncccc4F)c3=O)cc2F)s1] | light- yellow solid, yield: 71.9% | ¹H NMR (400 MHz, DMSO-d₆) δ 8.39 (s, 2H), 7.95 (dd, J = 12.6, 2.5 Hz, 1H), 7.86-7.70 (m, 2H), 7.48 (ddd, J = 9.1, 6.9, 2.7 Hz, 2H), 5.14 (d, J = 1.8 Hz, 2H), 3.87 (s, 3H), 2.35 (s, 3H). | 460.1 |
| | | methyl | | | |
| | | 5-(2-fluoro-4-(4-((3-fluoropyridin- | | | |
| | | 2-yl)methyl-5-oxo-4,5-dihydro- | | | |
| | | 1H-1,2,4-triazol-1-yl)phenoxy)-4- | | | |
| | | methylthiazole-2-carboxylate | | | |
| Example 33 Method 3 | +++ | 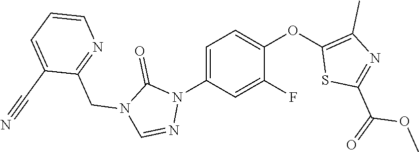 [SMILES: COC(=O)c1nc(C)c(Oc2ccc(-n3ncn(Cc4ncccc4C#N)c3=O)cc2F)s1] | light- yellow solid, yield: 73.7% | ¹H NMR (400 MHz, DMSO-d₆) δ 8.79 (dd, J = 5.0, 1.7 Hz, 1H), 8.45-8.34 (m, 2H), 7.95 (dd, J = 12.5, 2.5 Hz, 1H), 7.78 (dt, J = 9.1, 1.8 Hz, 1H), 7.58 (dd, J = 7.9, 4.9 Hz, 1H), 7.48 (t, J = 9.1 Hz, 1H), 5.29 (s, 2H), 3.87 (s, 3H), 2.35 (s, 3H). | 467.1 |
| | | methyl | | | |
| | | 5-(4-(4-((3-cyanopyridin-2-yl)meth- | | | |
| | | yl)-5-oxo-4,5-dihydro-1H-1,2,4- | | | |
| | | triazol-1-yl)-2-fluorophenoxy)-4- | | | |
| | | methylthiazole-2-carboxylate | | | |
| Example 34 Method 3 | +++ | 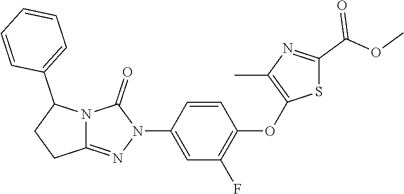 [SMILES: COC(=O)c1nc(C)c(Oc2ccc(-n3nc4n(c3=O)C(c3ccccc3)CC4)cc2F)s1] | yellow oil, yield: 70.5% | NA | 467.2 |
| | | methyl | | | |
| | | 5-(2-fluoro-4-(3-oxo-5-phenyl-6,7- | | | |
| | | dihydro-3H-pyrrolo[2,1-c][1,2,4] | | | |
| | | triazol-2(5H)-yl)phenoxy)-4-meth- | | | |
| | | ylthiazole-2-carboxylate | | | |
| Example 35 Method 3 Single unknown stereo- isomer | ++ | 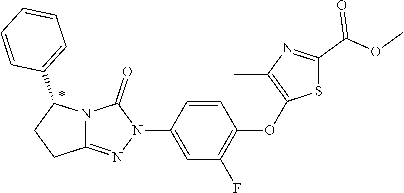 [SMILES: COC(=O)c1nc(C)c(Oc2ccc(-n3nc4n(c3=O)[C@@H](c3ccccc3)CC4)cc2F)s1] | white solid, yield: 21.0% | ¹H NMR (400 MHz, Chloroform-d) δ 7.93 (dd, J = 12.4, 2.4 Hz, 1H), 7.76 (dt, J = 9.2, 2.0 Hz, 1H), 7.44-7.32 (m, 3H), 7.27-7.23 (m, 2H), 7.11 (t, J = 8.8 Hz, 1H), 5.32-5.25 (m, 1H), 3.96 (s, 3H), 3.13-2.99 (m, 2H), 2.99-2.87 (m, 1H), 2.59-2.45 (m, 1H), 2.43 (s, 3H). | 467.2 |
| | | methyl | | | |
| | | (R)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methylthiazole-2-carboxylate | | | |
| Example 36 Method 3 Single unknown stereo- isomer | ++++ | 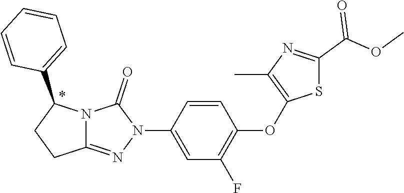 [SMILES: COC(=O)c1nc(C)c(Oc2ccc(-n3nc4n(c3=O)[C@H](c3ccccc3)CC4)cc2F)s1] | white solid, yield: 20.0% | ¹H NMR (400 MHz, Chloroform-d) δ 7.93 (dd, J = 12.4, 2.4 Hz, 1H), 7.76 (dt, J = 9.2, 2.0 Hz, 1H), 7.44-7.32 (m, 3H), 7.26-7.22 (m, 2H), 7.11 (t, J = 8.8 Hz, 1H), 5.28 (dd, J = 7.6, 4.0 Hz, 1H), 3.96 (s, 3H), 3.18-2.99 (m, 2H), 2.97-2.91 (m, 1H), 2.59-2.47 (m, 1H), 2.43 (s, 3H). | 467.2 |
| | | methyl | | | |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methylthiazole-2-carboxylate | | | |
| Example 685 Method 3 Single unknown stereo- isomer | +++ | 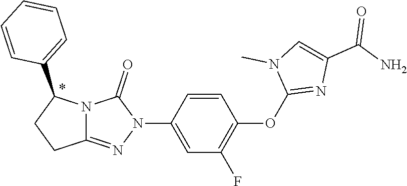 [SMILES: Cn1cc(C(N)=O)nc1Oc1ccc(-n2nc3n(c2=O)[C@H](c2ccccc2)CC3)cc1F] | white solid, yield: 14.09% | ¹H NMR (400 MHz, Chloroform-d) δ 7.90 (dd, J = 12.0, 2.8 Hz, 1H), 7.82- 7.75 (m, 1H), 7.48-7.29 (m, 6H), 7.26-7.23 (m, 1H), 6.63 (brs, 1H), 5.36-5.24 (m, 2H), 3.63 (s, 3H), 3.11-3.00 (m, 2H), 2.97-2.84 (m, 1H), 2.59-2.48 (m, 1H). | 435.4 |
| | | (S)-2-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-1- | | | |
| | | methyl-1H-imidazole-4-carboxamide | | | |
| Example 686 Method 3 Single unknown stereo- isomer | ++++ | 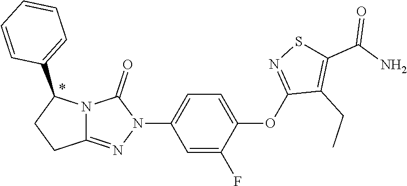 [SMILES: CCc1c(Oc2ccc(-n3nc4n(c3=O)[C@H](c3ccccc3)CC4)cc2F)nsc1C(N)=O] | white solid, yield: 5.99% | ¹H NMR (400 MHz, DMSO-d₆) δ 7.85 (dd, J = 12.4, 2.4 Hz, 1H), 7.76- 7.69 (m, 2H), 7.68-7.64 (m, 1H), 7.52 (t, J = 8.8 Hz, 1H), 7.43-7.36 (m, 2H), 7.37-7.30 (m, 3H), 5.33 (dd, J = 8.0, 4.8 Hz, 1H), 4.26-4.18 (m, 2H), 3.09-2.98 (m, 2H), 2.97- 2.90 (m, 1H), 2.39-2.30 (m, 1H), 0.91 (t, J = 7.2 Hz, 3H). | 466.4 |
| | | (S)-4-ethyl-3-(2-fluoro-4-(3-oxo- | | | |
| | | 5-phenyl-6,7-dihydro-3H-pyrrolo | | | |
| | | [2,1-c][1,2,4]triazol-2(5H)-yl)phe- | | | |
| | | noxy)isothiazole-5-carboxamide | | | |
| Example 687 Method 3 Single unknown stereo- isomer | ++++ | 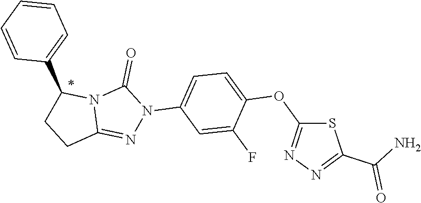 [SMILES: NC(=O)c1nnc(Oc2ccc(-n3nc4n(c3=O)[C@H](c3ccccc3)CC4)cc2F)s1] | white solid, yield: 6.62% | ¹H NMR (400 MHz, DMSO-d₆) δ 8.49 (brs, 1H), 8.13 (brs, 1H), 7.94 (dd, J = 12.4, 2.4 Hz, 1H), 7.85- 7.78 (m, 1H), 7.78-7.71 (m, 1H), 7.43-7.31 (m, 5H), 5.33 (dd, J = 7.6, 4.8 Hz, 1H), 3.09-2.99 (m, 2H), 2.98-2.88 (m, 1H), 2.39-2.31 (m, 1H). | 439.2 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-1,3,4- | | | |
| | | thiadiazole-2-carboxamide | | | |
| Example 688 Method 3 Single unknown stereo- isomer | +++ | 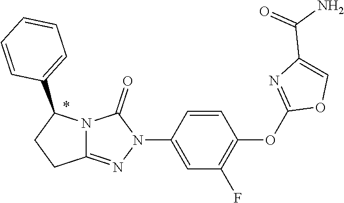 [SMILES: NC(=O)c1coc(Oc2ccc(-n3nc4n(c3=O)[C@H](c3ccccc3)CC4)cc2F)n1] | white solid, yield: 9.57% | ¹H NMR (400 MHz, DMSO-d₆) δ 8.30 (brs, 1H), 7.94-7.86 (m, 1H), 7.84-7.74 (m, 2H), 7.49-7.44 (m, 2H), 7.43-7.37 (m, 2H), 7.36-7.31 (m, 3H), 5.33 (dd, J = 7.6, 4.8 Hz, 1H), 3.10-2.98 (m, 2H), 2.96-2.87 (m, 1H), 2.42-2.28 (m, 1H). | 422.1 |
| | | (S)-2-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)oxa- | | | |
| | | zole-4-carboxamide | | | |
| Example 689 Method 3 Single unknown stereo- isomer | ++++ | 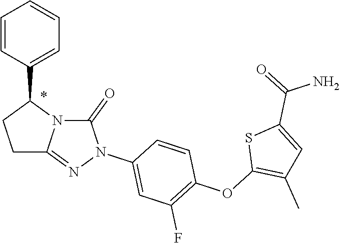 [SMILES: Cc1cc(C(N)=O)sc1Oc1ccc(-n2nc3n(c2=O)[C@H](c2ccccc2)CC3)cc1F] | white solid, yield: 12.03% | ¹H NMR (400 MHz, DMSO-d₆) δ 7.86 (dd, J = 12.8, 2.4 Hz, 2H), 7.69 (dt, J = 9.2, 1.6 Hz, 1H), 7.49 (s, 1H), 7.43-7.37 (m, 2H), 7.32 (q, J = 11.2, 7.2 Hz, 5H), 5.32 (dd, J = 8.0, 4.8 Hz, 1H), 3.08-2.97 (m, 2H), 2.96-2.86 (m, 1H), 2.36-2.30 (m, 1H), 2.07 (s, 3H). | 451.1 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | mthylthiophene-2-carboxamide | | | |
| Example 690 Method 3 Single unknown stereo- isomer | ++++ | 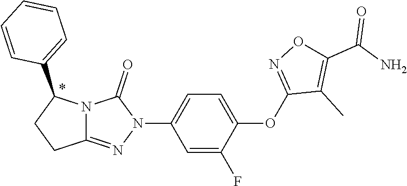 [SMILES: Cc1c(Oc2ccc(-n3nc4n(c3=O)[C@H](c3ccccc3)CC4)cc2F)noc1C(N)=O] | white solid, yield: 7.09% | ¹H NMR (400 MHz, DMSO-d₆) δ 8.21 (brs, 1H), 7.96 (brs, 1H), 7.88 (dd, J = 12.4, 2.4 Hz, 1H), 7.79- 7.71 (m, 1H), 7.64-7.55 (m, 1H), 7.44-7.37 (m, 2H), 7.37-7.30 (m, 3H), 5.33 (dd, J = 7.6, 4.8 Hz, 1H), 3.10-2.88 (m, 3H), 2.40-2.28 (m, 1H), 2.19 (s, 3H). | 436.1 |
| | | (S)-3-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methylisoxazole-5-carboxamide | | | |
| Example 691 Method 3 Single unknown stereo- isomer | ++ | 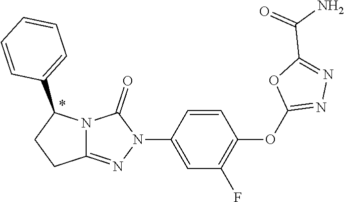 [SMILES: NC(=O)c1nnc(Oc2ccc(-n3nc4n(c3=O)[C@H](c3ccccc3)CC4)cc2F)o1] | white solid, yield: 2.89% | ¹H NMR (400 MHz, DMSO-d₆) δ 8.57 (brs, 1H), 8.18 (brs, 1H), 7.99- 7.91 (m, 1H), 7.77-7.71 (m, 1H), 7.71-7.66 (m, 1H), 7.43-7.37 (m, 2H), 7.36-7.30 (m, 3H), 5.33 (dd, J = 7.6, 5.2 Hz, 1H), 3.09-2.98 (m, 2H), 2.97-2.89 (m, 1H), 2.38-2.27 (m, 1H). | 423.1 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-1,3,4- | | | |
| | | oxadiazole-2-carboxamide | | | |
| Example 692 Method 3 Single unknown stereo- isomer | ++++ | 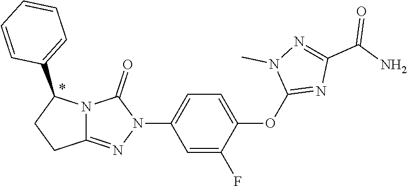 [SMILES: Cn1nc(C(N)=O)nc1Oc1ccc(-n2nc3n(c2=O)[C@H](c2ccccc2)CC3)cc1F] | white solid, yield: 39% | ¹H NMR (400 MHz, Methanol-d₄) δ 7.93 (dd, J = 12.4, 2.4 Hz, 1H), 7.84 (dt, J = 8.8, 2.0 Hz, 1H), 7.60 (t, J = 8.8 Hz, 1H), 7.46-7.17 (m, 5H), 5.35 (dd, J = 7.8, 4.8 Hz, 1H), 3.91 (s, 3H), 3.12-2.91 (m, 3H), 2.54- 2.43 (m, 1H). | 436.1 |
| | | 5-[2-fluoro-4-[(S)-3-oxo-5-phenyl- | | | |
| | | 6,7-dihydro-5H-pyrrolo[2,1-c][1,2,4] | | | |
| | | triazol-2-yl]phenoxy]-1-meth- | | | |
| | | yl-1,2,4-triazole-3-carboxamide | | | |
| Example 693 Method 3 Single unknown stereo- isomer | ++++ | 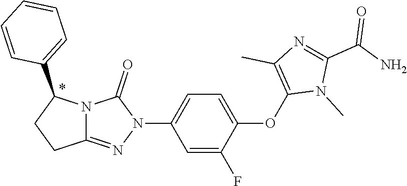 [SMILES: Cc1nc(C(N)=O)n(C)c1Oc1ccc(-n2nc3n(c2=O)[C@H](c2ccccc2)CC3)cc1F] | white solid, yield: 23% | ¹H NMR (400 MHz, Methanol-d₄) δ 7.77 (dd, J = 12.8, 2.4 Hz, 1H), 7.57 (dt, J = 9.2, 2.0 Hz, 1H), 7.37-7.12 (m, 5H), 6.88 (t, J = 9.2 Hz, 1H), 5.21 (dd, J = 7.6, 4.8 Hz, 1H), 3.21 (s, 3H), 3.05-2.75 (m, 3H), 2.44- 2.25 (m, 1H), 1.92 (s, 3H). | 449.2 |
| | | 5-[2-fluoro-4-[(5S)-3-oxo-5-phenyl- | | | |
| | | 6,7-dihydro-5H-pyrrolo[2,1-c][1,2,4] | | | |
| | | triazol-2-yl]phenoxy]-1,4-di- | | | |
| | | methyl-imidazole-2-carboxamid | | | |
| Example 694 Method 3 Single unknown stereo- isomer | ++++ | 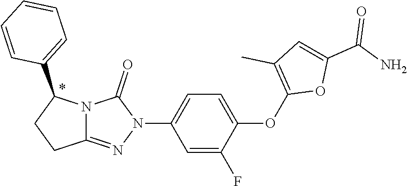 [SMILES: Cc1cc(C(N)=O)oc1Oc1ccc(-n2nc3n(c2=O)[C@H](c2ccccc2)CC3)cc1F] | White solid, yield: 17% | ¹H NMR (400 MHz, Methanol-d₄) 7.75 (dd, J = 12.8, 2.4 Hz, 1H), 7.61 (dt, J = 9.2, 2.0 Hz, 1H), 7.35-7.15 (m, 5H), 7.01 (t, J = 9.2 Hz, 1H), 6.95 (s, 1H), 5.22 (dd, J = 8.0, 4.4 Hz, 1H), 3.04-2.79 (m, 3H), 2.38- 2.26 (m, 1H), 1.83 (s, 3H). | 435.1 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methylfuran-2-carboxamide | | | |
| Example 695 Method 3 Single unknown stereo- isomer | ++++ | 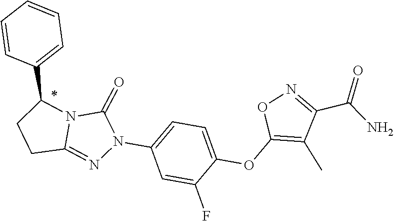 [SMILES: Cc1c(C(N)=O)noc1Oc1ccc(-n2nc3n(c2=O)[C@H](c2ccccc2)CC3)cc1F] | white solid, yield: 37% | ¹H NMR (400 MHz, DMSO-d₆) δ 8.05 (brs, 1H), 7.91 (dd, J = 12.8, 2.8 Hz, 1H), 7.81 (brs, 1H), 7.75 (ddd, J = 9.2, 2.4, 1.2 Hz, 1H), 7.49 (t, J = 9.2 Hz, 1H), 7.43-7.28 (m, 5H), 5.32 (dd, J = 8.0, 4.8 Hz, 1H), 3.09- 2.96 (m, 2H), 2.95-2.86 (m, 1H), 2.38-2.30 (m, 1H), 1.93 (s, 3H). | 436.3 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methylisoxazole-3-carboxamide | | | |
| Example 696 Method 3 Single unknown stereo- isomer | ++++ | 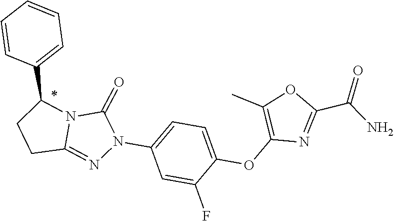 [SMILES: Cc1oc(C(N)=O)nc1Oc1ccc(-n2nc3n(c2=O)[C@H](c2ccccc2)CC3)cc1F] | white solid, yield: 21.2% | ¹H NMR (400 MHz, Chloroform-d) δ 7.85 (dd, J = 12.4, 2.8 Hz, 1H), 7.70 (dt, J = 9.0, 2.0 Hz, 1H), 7.43-73.0 (m, 3H), 7.26-7.22 (m, 2H), 7.09 (t, J = 8.8 Hz, 1H), 6.73 (brs, 1H), 5.78 (brs, 1H), 5.28 (dd, J = 7.6, 4.4 Hz, 1H), 3.11-2.98 (m, 2H), 2.97-2.87 (m, 1H), 2.57-2.46 (m, 1H), 2.38 (s, 3H). | 436.4 |
| | | (S)-4-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-5- | | | |
| | | methyloxazole-2-carboxamide | | | |
| Example 697 Method 3 Single unknown stereo- isomer | ++++ | 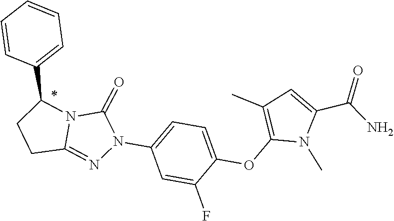 [SMILES: Cc1cc(C(N)=O)n(C)c1Oc1ccc(-n2nc3n(c2=O)[C@H](c2ccccc2)CC3)cc1F] | white solid, yield: 31.7% | ¹H NMR (400 MHz, Chloroform-d) δ 7.86 (d, = 12.8 Hz, 1H), 7.60 (d, J = 9.6 Hz, 1H), 7.43-7.31 (m, 3H), 7.26-7.24 (m, 2H), 7.00 (brs, 1H), 6.68 (t, J = 8.8 Hz, 1H), 6.44 (s, 1H), 5.37 (brs, 1H), 5.28 (dd, J = 8.0, 4.4 Hz, 1H), 3.73 (s, 3H), 3.12-2.97 (m, 2H), 2.96-2.84 (m, 1H), 2.55-2.45 (m, 1 H), 1.85 (s, 3H). | 448.3 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-1,4- | | | |
| | | dimethyl-1H-pyrrole-2-carboxa- | | | |
| | | mide | | | |
| Example 698 Method 3 Single unknown stereo- isomer | ++++ | 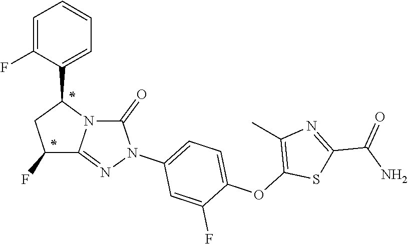 [SMILES: Cc1nc(C(N)=O)sc1Oc1ccc(-n2nc3n(c2=O)[C@H](c2ccccc2F)C[C@@H]3F)cc1F] | white solid, yield: 2.8% | ¹H NMR (400 MHz, Chloroform-d) δ 7.94 (dd, J = 12.4, 2.4 Hz, 1H), 7.77 (d, J = 8.8 Hz, 1H), 7.38-7.32 (m, 1H), 7.23-7.06 (m, 4H), 7.00 (brs, 1H), 5.84 (dd, J = 54.8, 6.4 Hz, 1H), 5.70-5.55 (m, 2H), 3.41-3.34 (m, 1H), 2.78 (dd, J = 23.2, 15.6 Hz, 1H), 2.36 (s, 3H). | 488.2 |
| | | 5-(2-fluoro-4-((5S,7S)-7-fluoro-5- | | | |
| | | (2-fluorophenyl)-3-oxo-6,7-dihy- | | | |
| | | dro-3H-pyrrolo[2,1-c][1,2,4]triazol- | | | |
| | | 2(5H)-yl)phenoxy)-4-methylthia- | | | |
| | | zole-2-carboxamide | | | |
| Example 699 Method 3 Single unknown stereo- isomer | ++++ | 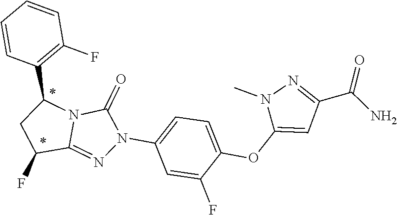 [SMILES: Cn1nc(C(N)=O)cc1Oc1ccc(-n2nc3n(c2=O)[C@H](c2ccccc2F)C[C@@H]3F)cc1F] | white solid, yield: 1.89% | ¹H NMR (400 MHz, Chloroform-d) δ 7.96 (dd, J = 12.0, 2.4 Hz, 1H), 7.80 (dt, J = 9.2, 2.0 Hz, 1H), 7.40-7.30 (m, 1H), 7.29-7.23 (m, 2H), 7.21- 7.10 (m, 2H), 6.70 (brs, 1H), 6.07 (s, 1H), 5.94-5.74 (m, 1H), 5.63 (dt, J = 8.8, 2.4 Hz, 1H), 5.38 (brs, 1H), 3.83 (s, 3H), 3.52-3.32 (m, 1H), 2.85-2.71 (m, 1H) | 471.1 |
| | | 5-(2-fluoro-4-((5S,7S)-7-fluoro-5- | | | |
| | | (2-fluorophenyl)-3-oxo-6,7-dihy- | | | |
| | | dro-3H-pyrrolo[2,1-c][1,2,4]triazol- | | | |
| | | 2(5H)-yl)phenoxy)-1-methyl-1H- | | | |
| | | pyrazole-3-carboxamide | | | |
| Example 700 Method 3 Single unknown stereo- isomer | ++++ | 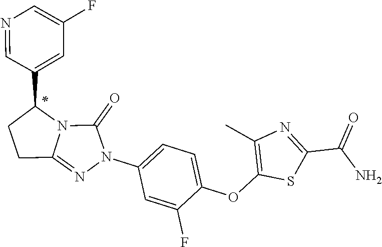 [SMILES: Cc1nc(C(N)=O)sc1Oc1ccc(-n2nc3n(c2=O)[C@H](c2cncc(F)c2)CC3)cc1F] | White solid, yield: 14.8% | ¹H NMR (400 MHz, DMSO-d₆) δ 8.57 (d, J = 2.8 Hz, 1H), 8.54 (t, J = 1.8 Hz, 1H), 8.09 (s, 1H), 7.92-7.83 (m, 2H), 7.79 (s, 1H), 7.72-7.66 (m, 1H), 7.38 (t, J = 9.1 Hz, 1H), 5.42 (t, J = 6.7 Hz, 1H), 3.11-3.00 (m, 2H), 2.98-2.82 (m, 1H), 2.48-2.36 (m, 1H), 2.31 (s, 3H). | 471.4 |
| | | (S)-5-(2-fluoro-4-(5-(5-fluoropyri- | | | |
| | | din-3-yl)-3-oxo-6,7-dihydro-3H-pyr- | | | |
| | | rolo[2,1-c][1,2,4]triazol-2(5H)- | | | |
| | | yl)phenoxy)-4-methylthiazole-2-car- | | | |
| | | boxamide | | | |
| Example 701 Method 3 Single unknown stereo- isomer | ++++ | 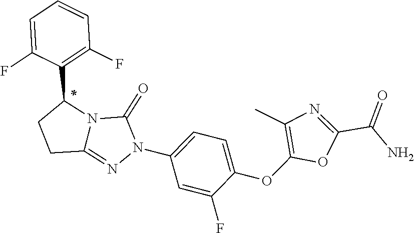 [SMILES: Cc1nc(C(N)=O)oc1Oc1ccc(-n2nc3n(c2=O)[C@H](c2c(F)cccc2F)CC3)cc1F] | white solid, yield: 29% | ¹H NMR (400 MHz, Chloroform-d) δ 7.87 (dd, J = 12.4, 2.4 Hz, 1H), 7.69 (dt, J = 9.2, 2.0 Hz, 1H), 7.37-7.28 (m, 1H), 7.09 (t, J = 8.8 Hz, 1H), 6.94 (t, J = 8.4 Hz, 2H), 6.71 (brs, 1H), 5.62 (dd, J = 8.8, 4.8 Hz, 1H), 5.43 (brs, 1H), 3.20-3.05 (m, 2H), 3.02-2.96 (m, 1H), 2.72-2.63 (m, 1H), 2.11 (s, 3H). | 472.3 |
| | | (S)-5-(4-(5-(2,6-difluorophenyl)- | | | |
| | | 3-oxo-6,7-dihydro-3H-pyrrolo[2,1-c] | | | |
| | | [1,2,4]triazol-2(5H)-yl)-2-flu- | | | |
| | | orophenoxy)-4-methyloxazole-2- | | | |
| | | carboxamide | | | |
| Example 702 Method 3 Single unknown stereo- isomer | ++++ | 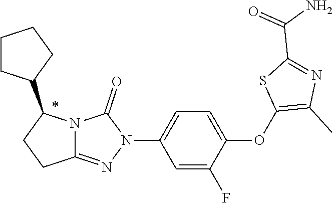 [SMILES: Cc1nc(C(N)=O)sc1Oc1ccc(-n2nc3n(c2=O)[C@H](C2CCCC2)CC3)cc1F] | white solid, yield: 9.9% | ¹H NMR (400 MHz, CDCl₃-d) δ 7.91 (dd, J = 12.4, 2.5 Hz, 1H), 7.73 (dd, J = 9.0, 1.7 Hz, 1H), 7.08 (t, J = 8.9 Hz, 1H), 6.98 (d, J = 10.5 Hz, 1H), 5.43 (s, 1H), 4.22 (td, J = 7.6, 3.4 Hz, 1H), 2.98-2.72 (m, 2H), 2.68-2.54 (m, 1H), 2.45-2.23 (m, 5H), 1.90 (dd, J = 11.8, 4.4 Hz, 1H), 1.80- 1.47 (m, 6H), 1.42-1.29 (m, 1H). | 444.3 |
| | | (S)-5-(4-(5-cyclopentyl-3-oxo-6,7- | | | |
| | | dihydro-3H-pyrrolo[2,1-c][1,2,4] | | | |
| | | triazol-2(5H)-yl)-2-fluorophenox- | | | |
| | | y)-4-methylthiazole-2-carboxamide | | | |

### Example 37: Method 4

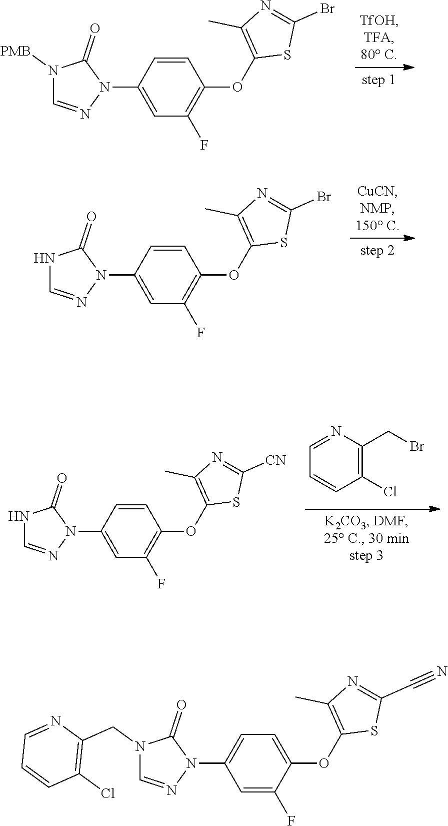

### Step 1: 2-(4-((2-bromo-4-methylthiazol-5-yl)oxy)-3-fluorophenyl)-2,4-dihydro-3H-1,2,4-triazol-3-one

A solution of methyl 2-(4-((2-bromo-4-methylthiazol-5-yl)oxy)-3-fluorophenyl)-4-(4-methoxybenzyl)-2,4-dihydro-3H-1,2,4-triazol-3-one (3.192 g, 6.5 mmol), TfOH (2.87 g, 19.0 mmol) in TFA (15 mL) was stirred at 80° C. for 16 h. The resulted solution was diluted with EA (150 mL), washed with water (150 mL) and brine (1000 mL), dried over Na₂SO₄, concentrated and purified by Combi-Flash with EA: PE=0:1˜2:1 to obtain methyl 5-(2-fluoro-4-(5-oxo-4, 5-dihydro-1H-1,2,4-triazol-1-yl)phenoxy)-4-methylthiazole-2-carboxylate (1.445 g, yield: 60%) as a green solid. LCMS (m/z): 371.3[M+H]⁺.

### Step 2: 5-(2-fluoro-4-(5-oxo-4,5-dihydro-1H-1,2,4-triazol-1-yl)phenoxy)-4-methylthiazole-2-carbonitrile

2-(4-((2-bromo-4-methylthiazol-5-yl)oxy)-3-fluorophenyl)-2,4-dihydro-3H-1,2,4-triazol-3-one (800 mg, 2.155 mmoL) and CuCN (231.6 mg, 2.586 mmoL) were dissolved in 6 mL NMP. The reactions mixture was stirred at 150° C. for 2h. Water was added to the solution and extracted with EA (30 mL×3). The organic layers were combined and evaporated to dryness and purified by C₁₈ column to give 5-(2-fluoro-4-(5-oxo-4,5-dihydro-1H-1,2,4-triazol-1-yl)phenoxy)-4-methylthiazole-2-carbonitr ile as off-white solid (400 mg, yield: 58.5%). LCMS (m/z): 318.3[M+H]⁺.

### Step 3: 5-(4-(4-((3-chloropyridin-2-yl)methyl)-5-oxo-4,5-dihydro-1H-1,2,4-triazol-1-yl)-2-fluorophenoxy)-4-methylthiazole-2-carbonitrile

To a solution of 2-(bromomethyl)-3-chloro-pyridine (1.00 eq, 200 mg, 0.969 mmol) in DMF (5 mL) was added K₂CO₃ (3.00 eq, 401 mg, 2.91 mmol), 5-[2-fluoro-4-(5-oxo-4H-1,2,4-triazol-1-yl)phenoxy]-4-methyl-thiazole-2-carbonitrile (1.00 eq, 307 mg, 0.969 mmol), the mixture was stirred for 30 min at 25° C. under nitrogen. The reaction mixture was concentrated to dryness and the residue was taken up in EA (50 mL) and the organics washed with 2×20 mL water then 1×20 mL saturated brine solution. The organics were then separated and dried (MgSO₄) before concentration to dryness. The crude was then purified by flash column chromatography eluted with 50% ACN in H₂O. The desired fractions were concentrated to dryness in vacuo to afford 5-[4-[4-[(3-chloro-2-pyridyl)methyl]-5-oxo-1,2,4-triazol-1-yl]-2-fluoro-phenoxy]-4-met hyl-thiazole-2-carbonitrile (80 mg, 0.168 mmol, 17.34% yield) as a white solid. MS (m/z): 443.2 [M+H]⁺. ¹H NMR (400 MHz, Chloroform-d) δ 8.49-8.43 (m, 1H), 8.02 (dd, J=12.4, 2.4 Hz, 1H), 7.92-7.84 (m, 1H), 7.80-7.71 (m, 2H), 7.29-7.24 (m, 1H), 7.17 (t, J=8.8 Hz, 1H), 5.17 (s, 2H), 2.44 (s, 3H).

### Example 38 was Synthesized Using a Similar Method to that Used in Example 37

| Compound | | | Appear- | | |
|---|---|---|---|---|---|
| No. | Activi- | | ance | | MS (m/z) |
| Method | ty | Structure and Name | and Yield | ¹H NMR Data | [M + H]⁺ |
| Example 37 Method 4 | ++++ | 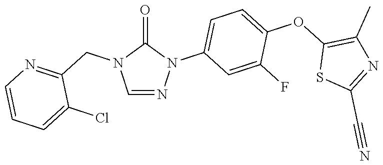 [SMILES: Cc1nc(C#N)sc1Oc1ccc(-n2ncn(Cc3ncccc3Cl)c2=O)cc1F] | white solid, yield: 17.34% | ¹H NMR (400 MHz, Chloroform-d) δ 8.49-8.43 (m, 1H), 8.02 (dd, J = 12.4, 2.4 Hz, 1H), 7.92-7.84 (m, 1H), 7.80-7.71 (m, 2H), 7.29-7.24 (m, 1H), 7.17 (t, J = 8.8 Hz, 1H), 5.17 (s, 2H), 2.44 (s, 3H). | 443.2 |
| | | 5-(4-(4-((3-chloropyridin-2-yl)meth- | | | |
| | | yl)-5-oxo-4,5-dihydro-1H-1,2,4- | | | |
| | | triazol-1-yl)-2-fluorophenoxy)-4- | | | |
| | | methylthiazole-2-carbonitrile | | | |
| Example 38 Method 4 | ++++ | 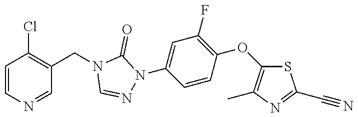 [SMILES: Cc1nc(C#N)sc1Oc1ccc(-n2ncn(Cc3cnccc3Cl)c2=O)cc1F] | white solid, yield: 23.5% | ¹H NMR (400 MHz, DMSO-d₆) δ 8.64 (s, 1H), 8.55 (d, J = 5.2 Hz, 1H), 8.40 (s, 1H), 7.99-7.86 (m, 1H), 7.80-7.67 (m, 1H), 7.62 (d, J = 5.2 Hz, 1H), 7.52 (t, J = 9.2 Hz, 1H), 5.04 (s, 2H), 2.36 (s, 3H). | 443.3 |
| | | 5-(4-(4-((4-chloropyridin-3-yl)meth- | | | |
| | | yl)-5-oxo-4,5-dihydro-1H-1,2,4- | | | |
| | | triazol-1-yl)-2-fluorophenoxy)-4- | | | |
| | | methylthiazole-2-carbonitrile | | | |

### Example 39: Method 5

### 3-(5-(4-(4-(2,6-difluorobenzyl)-5-oxo-4,5-dihydro-1H-1,2,4-triazol-1-yl)phenoxy)-4-methyl thiazol-2-yl)cyclobutane-1-carbonitrile

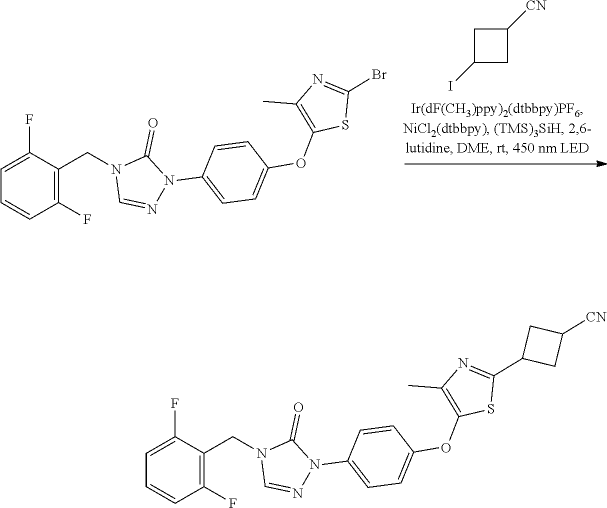 [SMILES: Cc1nc(Br)sc1Oc1ccc(-n2ncn(Cc3c(F)cccc3F)c2=O)cc1 ; N#CC1CC(I)C1 ; Cc1nc(C2CC(C#N)C2)sc1Oc1ccc(-n2ncn(Cc3c(F)cccc3F)c2=O)cc1]

To an 8 mL vial equipped with a stir bar was added photocatalyst Ir[dF(CF₃)ppy]₂ (dtbbpy)PF₆ (1.51 mg, 10.4 mol, 0.05 equiv.), 2-(4-((2-bromo-4-methylthiazol-5-yl)oxy)phenyl)-4-(2,6-difluorobenzyl)-2,4-dihydro-3H-1,2,4-triazol-3-one (100 mg, 0.21 mmol), 3-iodocyclobutane-1-carbonitrile (41.63 mg, 0.2 mmol), tris(trimethylsilyl)silane (103.47 mg, 0.42 mmol). The vial was sealed and placed under nitrogen before DME (4 mL) was added. To a separate vial was added NiCl2(dtbbpy) (4.15 mg, 10.4 μmol) and 2,6-lutidine (44.6 mg, 0.41 mmol). The catalyst vial was sealed, purged with nitrogen and then to it was added DME (2 mL). The precatalyst solution was sonicated or stirred for 5 minutes, after which, 1 mL (0.5 mol % catalyst, 2.5 μmol, 0.005 equiv.) was syringed into the reaction vessel. The solution was degassed by sparging with nitrogen while stirring for 10 minutes before sealing with Parafilm. The reaction was stirred and irradiated with a 34 W blue LED lamp (7 cm away), with cooling fan to keep the reaction temperature at 25° C. for 12 hours. The resulted mixture was diluted with water (5 mL). The resulted mixture was extracted with EtOAc (3×10 mL). The combined Organic layers were washed with brine (4 mL), dried over anhydrous Na₂SO₄, and after filtration, the filtrate was concentrated under reduced pressure. The residue was purified by flash chromatography with the following conditions: column: Gemini 5u C18 150×21.2 mm; Mobile phase B: ACN; Flow rate: 20 mL/min; Gradient: 400% B-60% B in 20 min; Detector: 214 nm. Concentrated under reduced pressure to afford the titled compound (1.6 mg, 1.6% yield) as a white solid. LC-MS (m/z): 480.1 [M+H]+. 1H NMR (400 MHz, CDCl3) δ ppm7.97 (d, J=8.4 Hz, 2H), 7.57 (s, 1H), 7.32-7.40 (m, 1H), 7.04-7.06 (m, 2H), 6.95-6.99 (m, 2H), 4.97 (s, 2H), 4.08-4.06 (m, 1H), 3.12-3.18 (i, 1H), 2.91-2.98 (i, 2H), 2.71-2.64 (i, 2H), 2.35 (s, 3H).

### Examples (Compounds) 40-42 were Synthesized Using a Similar Method to that Used in Example 39

| Com- | | | Ap- | | |
|---|---|---|---|---|---|
| pound | | | pear- | | MS |
| No. | | | ance | | (m/z) |
| Meth- | Ac- | | and | | [M + |
| od | tivity | Structure and Name | Yield | ¹H NMR Data | H]⁺ |
| Exam- ple 39 Meth- od 5 | ++++ | 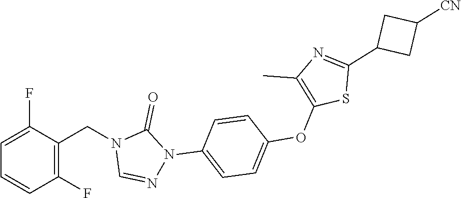 [SMILES: Cc1nc(C2CC(C#N)C2)sc1Oc1ccc(-n2ncn(Cc3c(F)cccc3F)c2=O)cc1] | white solid, yield: 1.6% | ¹H NMR (400 MHz, CDCl₃) δ 7.97 (d, J = 8.4 Hz, 2H), 7.57 (s, 1H), 7.32-7.40 (m, 1H), 7.04-7.06(m, 2H), 6.95-6.99 (m, 2H),4.97 (s, 2H), 4.08-4.06 (m, 1H), 3.12-3.18 (m, 1H), 2.91-2.98 (m, 2H), 2.71-2.64 (m, 2H), 2.35 (s, 3H). | 480.1 |
| | | 3-(5-(4-(4-(2,6-difluorobenzyl)-5- | | | |
| | | oxo-4,5-dihydro-1H-1,2,4-triazol- | | | |
| | | 1-yl)phenoxy)-4-methylthiazol-2- | | | |
| | | yl)cyclobutane-1-carbonitrile | | | |
| Exam- ple 40 Meth- od 5 | ++++ | 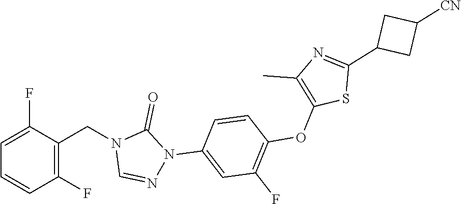 [SMILES: Cc1nc(C2CC(C#N)C2)sc1Oc1ccc(-n2ncn(Cc3c(F)cccc3F)c2=O)cc1F] | white solid, yield: 5.0% | ¹H NMR (400 MHz, CDCl₃) δ 7.90 (dd, J = 12.2, 2.3 Hz, 1H), 7.74 (d, J = 8.2 Hz, 1H), 7.60 (s, 1H), 7.39-7.36 (m, 1H), 7.00 (dd, J = 17.1, 9.5 Hz, 3H), 4.98 (s, 2H), 4.02 (d, J = 8.0 Hz, 1H), 3.29 (s, 1H), 2.82 (d, J = 8.1 Hz, 4H), 2.32 (s, 3H). | 498.1 |
| | | 3-(5-(4-(4-(2,6-difluorobenzyl)-5- | | | |
| | | oxo-4,5-dihydro-1H-1,2,4-triazol- | | | |
| | | 1-yl)-2-fluorophenoxy)-4-methyl- | | | |
| | | thiazol-2-yl)cyclobutane-1- | | | |
| | | carbonitrile | | | |
| Exam- ple 41 Meth- od 5 | ++++ | 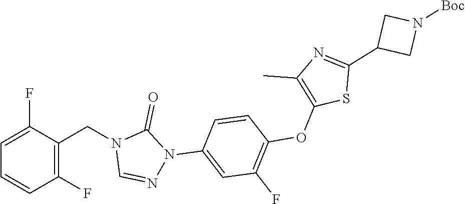 [SMILES: Cc1nc(C2CN(C(=O)OC(C)(C)C)C2)sc1Oc1ccc(-n2ncn(Cc3c(F)cccc3F)c2=O)cc1F] | white solid, yield: 4.3% | ¹H NMR (400 MHz, ) δ 7.89 (dd, J = 12.2, 2.5 Hz, 1H), 7.72-7.69 (m, 1H), 7.57 (s, 1H), 7.38-7.34 (m, 1H), 6.98 (td, J = 8.6, 4.8 Hz, 3H), 4.96 (s, 2H), 4.30 (dd, J = 11.1, 6.1 Hz, 2H), 4.11 (dd, J = 8.6, 6.0 Hz, 2H), 3.95 (ddd, J = 8.7, 7.3, 4.4 Hz, 1H), 2.28 (s, 3H), 1.43 (s, 9H). | 574.2 |
| | | tert-butyl | | | |
| | | 3-(5-(4-(4-(2,6-difluorobenzyl)-5- | | | |
| | | oxo-4,5-dihydro-1H-1,2,4-triazol- | | | |
| | | 1-yl)-2-fluorophenoxy)-4-methyl- | | | |
| | | thiazol-2-yl)azetidine-1- | | | |
| | | carboxylate | | | |
| Exam- ple 42 Meth- od 5 | ++++ | 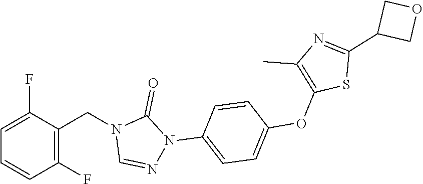 [SMILES: Cc1nc(C2COC2)sc1Oc1ccc(-n2ncn(Cc3c(F)cccc3F)c2=O)cc1] | white solid, yield: 14.3% | ¹H NMR (400 MHz, CDCl₃) δ 7.94-7.90 (m, 2H), 7.58 (s, 1H), 7.38-7.34 (m, 1H), 7.04-6.99 (m, 4H), 5.06 (dd, J = 8.4, 6.3 Hz, 2H), 4.98 (s, 2H), 4.88 (t, J = 6.5 Hz, 2H), 4.54 (td, J = 8.3, 4.1 Hz, 1H), 2.27 (s, 3H) | 457.1 |
| | | 4-(2,6-difluorobenzyl)-2-(4-((4- | | | |
| | | methyl-2-(oxetan-3-yl)thiazol-5-yl) | | | |
| | | oxy)phenyl)-2,4-dihydro-3H-1,2, | | | |
| | | 4-triazol-3-one | | | |

### Example 43: Method 6

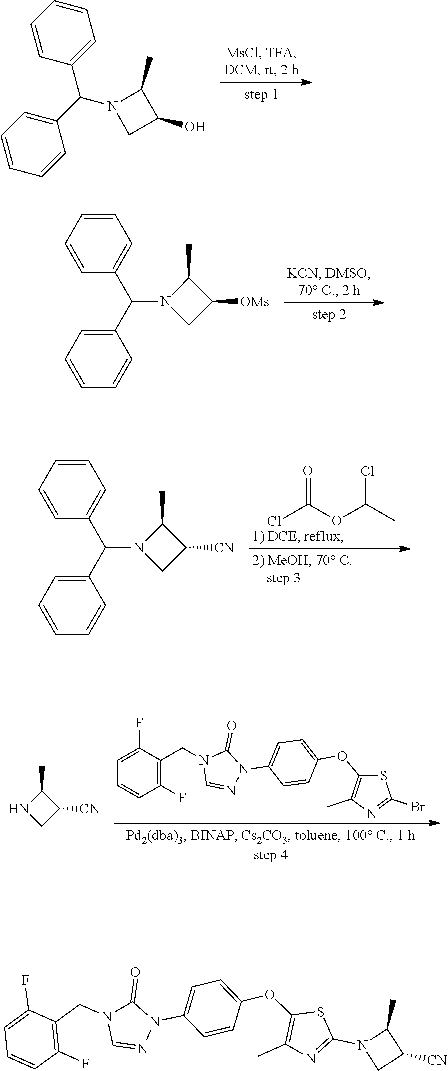

### Step 1: (2S,3S)-1-benzhydryl-2-methylazetidin-3-yl methanesulfonate

To a stirred solution of (2S,3S)-1-benzhydryl-2-methylazetidin-3-ol (1 g, 3.95 mmol), MsCl (533.52 mg, 4.68 mmol) and TEA (472.68 mg, 4.68 mmol) in DCM (50 mL). The resulted mixture was stirred for additional 1 h at 25° C.. The resulted mixture was diluted with 1N HCl (10 mL). The organic was diluted with NaHCO₃ solvent. The mixture was extracted with EtOAc (3×30 mL). The combined Organic layers were dried over anhydrous Na₂SO₄, after filtration, the filtrate was concentrated under reduced pressure. No further purification was performed. (2S,3S)-1-benzhydryl-2-methylazetidin-3-yl methanesulfonate (800 mg, yield: 61.5%) as an oil. LC-MS (m/z): 332.1 [M+H]⁺.

### Step 2: (2S,3R)-1-benzhydryl-2-methylazetidine-3-carbonitrile

To a stirred solution of (2S,3S)-1-benzhydryl-2-methylazetidin-3-yl methanesulfonate (800 mg, 2.4 mmol), KCN (234 mg, 3.6 mmol) and TEA (363.6 mg, 3.6 mmol) in DMSO (50 mL). The resulted mixture was stirred for additional 2 h at 70° C. The resulted mixture was diluted with water (50 mL). The resulted mixture was extracted with EtOAc (3×50 mL). The combined organic layers were washed with brine (50 mL), dried over anhydrous Na₂SO₄, and after filtration, the filtrate was concentrated under reduced pressure. The residue was purified by flash chromatography (PE: EtOAc=1:1) to afford (2S,3R)-1-benzhydryl-2-methylazetidine-3-carbonitrile (410 mg, yield: 65.2%) as a yellow solid. LC-MS (m/z): 263.3 [M+H]⁺.

### Step 3: (2S,3R)-2-methylazetidine-3-carbonitrile

To a stirred solution of (2S,3R)-1-benzhydryl-2-methylazetidine-3-carbonitrile (200 mg, 0.76 mmol), 1-chloroethyl carbonochloridate (163.0 mg, 1.14 mmol) in DCE (20 mL). The resulted mixture was stirred for additional 12 h at 70° C.. The reaction mixture was concentrated under reduced pressure to give a residue. MeOH (20 mL) was added in mixture. The resulted mixture was stirred for additional 1 h at 70° C. The resulted mixture was diluted with water (50 mL). The resulted mixture was extracted with EtOAc (3×50 mL). The combined Organic layers were washed with brine (50 mL), dried over anhydrous Na₂SO₄, and after filtration, the filtrate was concentrated under reduced pressure. The residue was purified by flash chromatography (PE: EtOAc=1:1) to afford (2S,3R)-2-methylazetidine-3-carbonitrile (60 mg, yield: 82.3%) as a white solid. LC-MS (m/z): 97.07 [M+H]⁺.

### Step 4: (2S,3R)-1-(5-(4-(4-(2,6-difluorobenzyl)-5-oxo-4,5-dihydro-1H-1,2,4-triazol-1-yl) phenoxy)-4-methylthiazol-2-yl)-2-methylazetidine-3-carbonitrile

To a stirred solution of (2S,3R)-2-methylazetidine-3-carbonitrile (60 mg, 0.62 mmol), 2-(4-((2-bromo-4-methylthiazol-5-yl)oxy)phenyl)-4-(2,6-difluorobenzyl)-2,4-dihydro-3H-1,2,4-triazol-3-one (297.0 mg, 0.62 mmol), BINAP (38.5 mg, 0.06 mmol) Pd₂(dba)₃ (35.6 mg, 0.062 mmol) and Cs₂CO₃ (606.4 mg, 1.86 mmol) in Toluene (20 mL). The reaction mixture was stirred at 110° C. for 2 h. The resulted mixture was diluted with water (20 mL). The resulted mixture was extracted with EtOAc (3×30 mL). The combined organic layers were washed with brine (10 mL), dried over anhydrous Na₂SO₄, and after filtration, the filtrate was concentrated under reduced pressure. The residue was purified by flash chromatography with the following conditions: column: Gemini 5u C18 150×21.2 mm; Mobile phase B: ACN; Flow rate: 20 mL/min; Gradient: 40% B-60% B in 20 min; Detector: 214 nm. The desired fractions were concentrated under reduced pressure to afford the titled compound (28.8 mg, yield: 9.4%) as a white solid LC-MS (m/z): 495.1 [M+H]⁺. ¹H NMR (400 MHz, CDCl₃) δ 7.91 (d, J=8.1 Hz, 2H), 7.58 (s, 1H), 7.39-7.35 (m, 1H), 7.02-6.98 (m, 4H), 4.98 (s, 2H), 4.70 (s, 1H), 4.38 (d, J=30.9 Hz, 2H), 3.78 (d, J=19.8 Hz, 1H), 2.16 (s, 3H), 1.70 (s, 3H)

Examples 44-58 were synthesized using a similar method to that used in Example 43

| Com- | | | Ap- | | |
|---|---|---|---|---|---|
| pound | | | pear- | | MS |
| No. | | | ance | | (m/z) |
| Meth- | | | and | | [M + |
| od | Activity | Structure and Name | Yield | ¹H NMR Data | H]⁺ |
| Ex- am- ple 43 Meth- od 6 | ++++ | 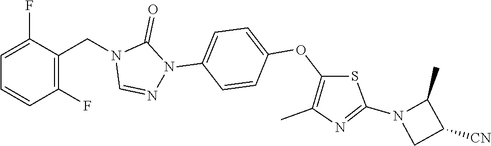 [SMILES: Cc1nc(N2C[C@@H](C#N)[C@@H]2C)sc1Oc1ccc(-n2ncn(Cc3c(F)cccc3F)c2=O)cc1] | white solid, yield: 9.4% | ¹H NMR (400 MHz, CDCl₃) δ 7.91 (d, J = 8.1 Hz, 2H), 7.58 (s, 1H), 7.39-7.35 (m, 1H), 7.02-6.98 (m, 4H), 4.98 (s, 2H), 4.70 (s, 1H), 4.38 (d, J = 30.9 Hz, | 495.1 |
| | | | | 2H), 3.78 (d, J = | |
| | | (2S,3R)-1-(5-(4-(4-(2,6-difluoro- | | 19.8 Hz, 1H), 2.16 | |
| | | benzyl)-5-oxo-4,5-dihydro-1H-1,2,4- | | (s, 3H), 1.70 (s, | |
| | | triazol-1-yl)phenoxy)-4-methyl- | | 3H) | |
| | | thiazol-2-yl)-2-methylazetidine-3- | | | |
| | | carbonitrile | | | |
| Ex- am- ple 44 Meth- od 6 | ++++ | 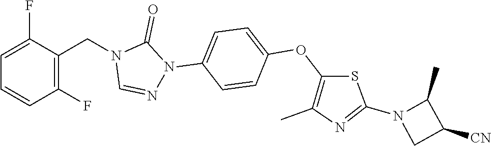 [SMILES: Cc1nc(N2C[C@H](C#N)[C@@H]2C)sc1Oc1ccc(-n2ncn(Cc3c(F)cccc3F)c2=O)cc1] | white solid, yield: 3% | ¹H NMR (400 MHz, CDCl₃) δ 7.94- 7.85 (m, 2H), 7.56 (s, 1H), 7.39-7.31 (m, 1H), 7.08-6.93 (m, 4H), 4.97 (s, 2H), 4.52-4.44 (m, 1H), 4.25 (m, 1H), 4.13 (t, J = 7.9 | 495.1 |
| | | | | Hz, 1H), 3.22 (q, | |
| | | (2S,3S)-1-(5-(4-(4-(2,6-difluoro- | | J = 15.8, 8.0 Hz, | |
| | | benzyl)-5-oxo-4,5-dihydro-1H-1,2, | | 1H), 2.11 (s, 3H), | |
| | | 4-triazol-1-yl)phenoxy)-4-methyl- | | 1.58 (d, J = 6.2 | |
| | | thiazol-2-yl)-2-methylazetidine-3- | | Hz, 3H). | |
| | | carbonitrile | | | |
| Ex- am- ple 45 Meth- od 6 | ++++ | 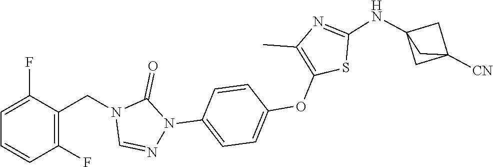 [SMILES: Cc1nc(NC23CC(C#N)(C2)C3)sc1Oc1ccc(-n2ncn(Cc3c(F)cccc3F)c2=O)cc1] | white solid, yield: 4.4% | ¹H NMR (400 MHz, CDCl₃) δ 7.92-7.83 (m, 2H), 7.55 (s, 1H), 7.42-7.29 (m, 1H), 7.07-6.94 (m, 4H), 4.97 (s, 2H), 2.11 (s, 3H), 2.36-2.20 (m, 2H), 2.16-2.11 (m, 2H), 2.08-1.98 (m, 2H). | 506.9 |
| | | 3-((5-(4-(4-(2,6-difluorobenzyl)-5- | | | |
| | | oxo-4,5-dihydro-1H-1,2,4-triazol- | | | |
| | | 1-yl)phenoxy)-4-methylthiazol-2- | | | |
| | | yl)amino)bicyclo[1.1.1]pentane- | | | |
| | | 1-carbonitrile | | | |
| Ex- am- ple 46 Meth- od 6 | ++++ | 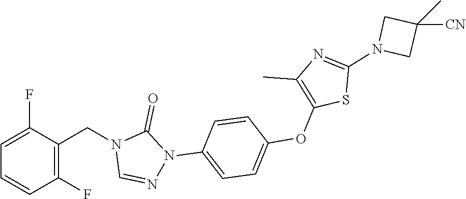 [SMILES: Cc1nc(N2CC(C)(C#N)C2)sc1Oc1ccc(-n2ncn(Cc3c(F)cccc3F)c2=O)cc1] | yellow solid, yield: 19% | ¹H NMR (400 MHz, CDCl₃) δ 7.90 (dd, J = 9.6, 2.5 Hz, 2H), 7.57 (s, 1H), 7.39-7.35 (m, 1H), 7.05-6.94 (m, 4H), 4.97 (s, 2H), 4.45 (s, 2H), 4.03 (s, 2H), 2.13 (s, 3H), 1.78 (s, 3H). | 495.1 |
| | | 1-(5-(4-(4-(2,6-difluorobenzyl)-5- | | | |
| | | oxo-4,5-dihydro-1H-1,2,4-triazol- | | | |
| | | 1-yl)phenoxy)-4-methylthiazol-2- | | | |
| | | yl)-3-methylazetidine-3- | | | |
| | | carbonitrile | | | |
| Ex- am- ple 47 Meth- od 6 | ++++ | 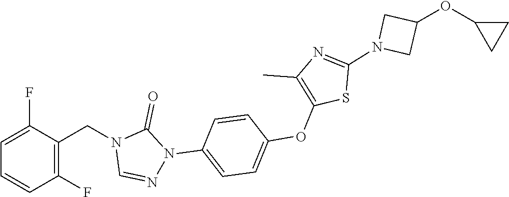 [SMILES: Cc1nc(N2CC(OC3CC3)C2)sc1Oc1ccc(-n2ncn(Cc3c(F)cccc3F)c2=O)cc1] | yellow solid, yield: 10% | ¹H NMR (400 MHz, CDCl₃) δ 7.87 (m, 2H), 7.56 (s, 1H), 7.36 (m, 1H), 7.04-6.95 (m, 4H), 4.97 (s, 2H), 4.57 (m, 1H), 4.28 (s, 2H), 4.01 (s, 2H), 3.29 (m, 1H), 2.10 (s, 3H), 0.62 (d, J = 3.8 Hz, 2H), 0.50 (m, 2H) | 512.1 |
| | | 2-(4-((2-(3-cyclopropoxyazetidin- | | | |
| | | 1-yl)-4-methylthiazol-5-yl)oxy) | | | |
| | | phenyl)-4-(2,6-difluorobenzyl)-2,4- | | | |
| | | dihydro-3H-1,2,4-triazol-3-one | | | |
| Ex- am- ple 48 Meth- od 6 | ++++ | 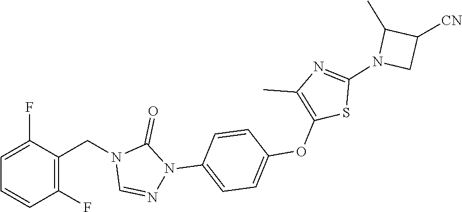 [SMILES: Cc1nc(N2CC(C#N)C2C)sc1Oc1ccc(-n2ncn(Cc3c(F)cccc3F)c2=O)cc1] | white solid, yield: 8.6% | ¹H NMR (400 MHz, CDCl₃) δ 7.92-7.83 (m, 2H), 7.55 (s, 1H), 7.42-7.29 (m, 1H), 7.07-6.94 (m, 4H), 4.97 (s, 2H), 4.58 (s, 1H), 4.33-4.20 (m, 2H), 3.67 (s,1H), 2.11 (s, 3H), 0.91-0.78 (m, 3H). | 495.1 |
| | | 1-(5-(4-(4-(2,6-difluorobenzyl)-5- | | | |
| | | oxo-4,5-dihydro-1H-1,2,4-triazol- | | | |
| | | 1-yl)phenoxy)-4-methylthiazol-2- | | | |
| | | yl)-2-methylazetidine-3- | | | |
| | | carbonitrile | | | |
| Ex- am- ple 49 Meth- od 6 | ++++ | 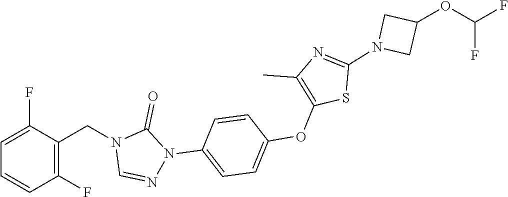 [SMILES: Cc1nc(N2CC(OC(F)F)C2)sc1Oc1ccc(-n2ncn(Cc3c(F)cccc3F)c2=O)cc1] | white solid, yield: 11% | ¹H NMR (400 MHz, DMSO-d₆) δ 8.28 (s, 1H), 7.87- 7.77 (m, 2H), 7.53-7.44 (m, 1H), 7.21-7.08 (m, 4H), 6.97 (s, 0.25H), 6.79 (s, 0.5H), 6.60 (s, 0.25H), 5.16-5.07 (m, 1H), 4.97 (s, 2H), 4.29 (dd, J = 9.0, | 522.0 |
| | | | | 7.3 Hz, 2H), 3.94 | |
| | | 4-(2,6-difluorobenzyl)-2-(4-((2-(3 | | (dd, J = 9.5, 4.0 | |
| | | -(difluoromethoxy)azetidin-1-yl)- | | Hz, 2H), 1.99 (s, 3H). | |
| | | 4-methylthiazol-5-yl)oxy)phenyl)- | | | |
| | | 2,4-dihydro-3H-1,2,4-triazol-3- | | | |
| | | one | | | |
| Ex- am- ple 50 Meth- od 6 | ++++ | 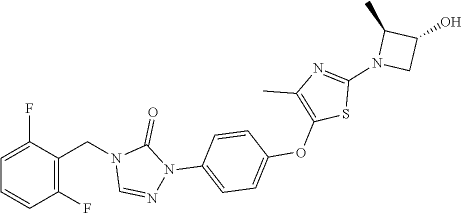 [SMILES: Cc1nc(N2C[C@@H](O)[C@@H]2C)sc1Oc1ccc(-n2ncn(Cc3c(F)cccc3F)c2=O)cc1] | white solid, yield: 0.96% | ¹H NMR (400 MHz, CDCl₃) δ 7.88 (d, J = 9.1 Hz, 2H), 7.56 (s, 1H), 7.39-7.34 (m, 1H), 7.06-6.96 (m, 4H), 4.97 (s, 2H), 4.32-4.15 (m, 4H), 2.13 (s, 3H), 1.50 (d, J = 6.3 Hz, 3H). | 486.0 |
| | | 4-(2,6-difluorobenzyl)-2-(4-((2- | | | |
| | | ((2S,3R)-3-hydroxy-2-methylazetidin- | | | |
| | | 1-yl)-4-methylthiazol-5-yl)oxy) | | | |
| | | phenyl)-2,4-dihydro-3H-1,2,4- | | | |
| | | triazol-3-one | | | |
| Ex- am- ple 51 Meth- od 6 | ++++ | 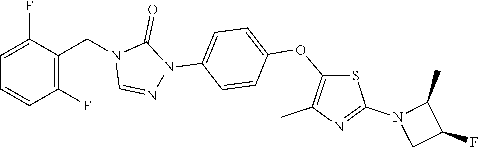 [SMILES: Cc1nc(N2C[C@H](F)[C@@H]2C)sc1Oc1ccc(-n2ncn(Cc3c(F)cccc3F)c2=O)cc1] | white solid, yield: 6.6% | ¹H NMR (400 MHz, DMSO-d₆) δ 8.28 (s, 1H), 7.90- 7.76 (m, 2H), 7.55-7.42 (m, 1H), 7.13 (ddd, J = 11.6, 9.2, 5.1 Hz, 4H), 4.97 (s, 2H), 4.15-4.03 (m, 2H), | 488.0 |
| | | | | 3.95-3.87 (m, | |
| | | 4-(2,6-difluorobenzyl)-2-(4-((2- | | 1H), 3.51 (dd, J = | |
| | | ((2S,3S)-3-fluoro-2-methylazetidin- | | 6.9, 5.2 Hz, 1H), | |
| | | 1-yl)-4-methylthiazol-5-yl)oxy) | | 1.99 (s, 3H), 1.35 | |
| | | phenyl)-2,4-dihydro-3H-1,2,4- | | (d, J = 6.4 Hz, | |
| | | triazol-3-one | | 3H). | |
| Ex- am- ple 52 Meth- od 6 | ++++ | 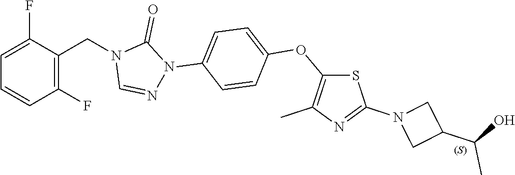 | white solid, yield: | ¹H NMR (400 MHz, CDCl₃) δ 7.88 (d, J = 9.1 Hz, 2H), 7.56 (s, 1H), 7.39-7.34 (m, 1H), 7.06-6.96 (m, 4H), 4.97 (s, 2H), 4.32-4.15 (m, 4H), 2.13 (s, 3H), 1.50 (d, J = 6.3 Hz, 3H). | 500.1 |
| | | (S)-4-(2,6-difluorobenzyl)-2-(4-((2- | | | |
| | | (3-(1-hydroxyethyl)azetidin-1-yl)- | | | |
| | | 4-methylthiazol-5-yl)oxy)phenyl)- | | | |
| | | 2,4-dihydro-3H-1,2,4-triazol- | | | |
| | | 3-one | | | |
| Ex- am- ple 53 Meth- od 6 | ++++ | 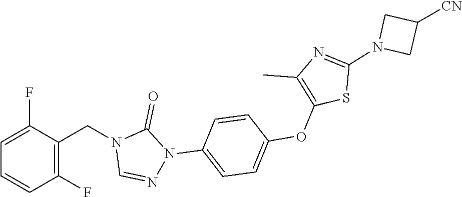 [SMILES: Cc1nc(N2CC(C#N)C2)sc1Oc1ccc(-n2ncn(Cc3c(F)cccc3F)c2=O)cc1] | white solid, yield: | ¹H NMR (400 MHz, CDCl₃) δ 7.93- 7.86 (m, 2H), 7.56 (s, 1H), 7.45- 7.31 (m, 1H), 7.11- 6.92 (m, 4H), 4.97 (s, 2H), 4.41- 4.22 (m, 4H), 3.72-3.63 (m, 1H), 2.10 (s, 3H). | 481.2 |
| | | 1-(5-(4-(4-(2,6-difluorobenzyl)-5- | | | |
| | | oxo-4,5-dihydro-1H-1,2,4-triazol- | | | |
| | | 1-yl)phenoxy)-4-methylthiazol-2- | | | |
| | | yl)azetidine-3-carbonitrile | | | |
| Ex- am- ple 54 Meth- od 6 | ++++ | 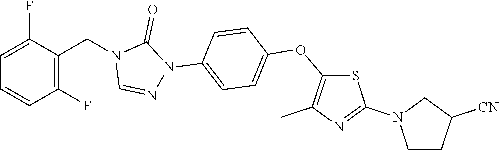 [SMILES: Cc1nc(N2CCC(C#N)C2)sc1Oc1ccc(-n2ncn(Cc3c(F)cccc3F)c2=O)cc1] | white solid, yield: | ¹H NMR (400 MHz, CDCl₃) δ 7.92- 7.85 (m, 2H), 7.56 (s, 1H), 7.40-7.32 (m, 1H), 7.09-6.94 (m, 4H), 4.97 (s, 2H), 3.90-3.48 (m, 4H), 3.35-3.21 (m, 1H), 2.56- 2.34 (m, 2H), 2.11 (s, 3H). | 495.2 |
| | | 1-(5-(4-(4-(2,6-difluorobenzyl)-5- | | | |
| | | oxo-4,5-dihydro-1H-1,2,4-triazol- | | | |
| | | 1-yl)phenoxy)-4-methylthiazol-2- | | | |
| | | yl)pyrrolidine-3-carbonitrile | | | |
| Ex- am- ple 55 Meth- od 6 | ++++ | 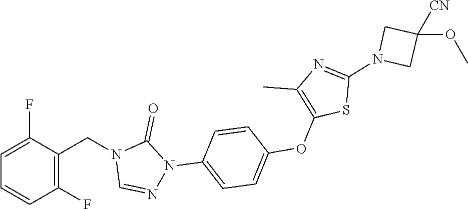 [SMILES: COC1(C#N)CN(c2nc(C)c(Oc3ccc(-n4ncn(Cc5c(F)cccc5F)c4=O)cc3)s2)C1] | white solid, yield: 20.9% | ¹H NMR (400 MHz, CDCl₃) δ 7.91- 7.86 (m, 2H), 7.56 (s, 1H), 7.37 (ddd, J = 8.4, 7.5, 4.2 Hz, 1H), 7.06- 6.95 (m, 4H), 4.97 (s, 2H), 4.45 (d, J = 8.9 Hz, 2H), 4.21 (d, J = 8.5 Hz, 2H), 3.49 (s, 3H), 2.12 (s, 3H). | 511.1 |
| | | 1-(5-(4-(4-(2,6-difluorobenzyl)-5- | | | |
| | | oxo-4,5-dihydro-1H-1,2,4-triazol- | | | |
| | | 1-yl)phenoxy)-4-methylthiazol-2- | | | |
| | | yl)-3-methoxyazetidine-3- | | | |
| | | carbonitrile | | | |
| Ex- am- ple 56 Meth- od 6 | ++++ | 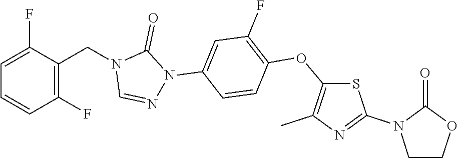 [SMILES: Cc1nc(N2CCOC2=O)sc1Oc1ccc(-n2ncn(Cc3c(F)cccc3F)c2=O)cc1F] | white solid, yield: 28.5% | ¹H NMR (400 MHz, CDCl₃) δ 7.88 (dd, J = 12.3, 2.5 Hz, 1H), 7.69 (ddd, J = 9.1, 2.5, 1.6 Hz, 1H), 7.57 (s, 1H), 7.40-7.31 (m, 1H), 7.01- 6.94 (m, 3H), 4.95 (s, 2H), 4.65- | 503.9 |
| | | | | 4.54 (m, 2H), 4.39- | |
| | | 3-(5-(4-(4-(2,6-difluorobenzyl)-5- | | 4.25 (m, 2H), | |
| | | oxo-4,5-dihydro-1H-1,2,4-triazol- | | 2.22 (s, 3H). | |
| | | 1-yl)-2-fluorophenoxy)-4-methyl- | | | |
| | | thiazol-2-yl)oxazolidin-2-one | | | |
| Ex- am- ple 57 Meth- od 6 | ++++ | 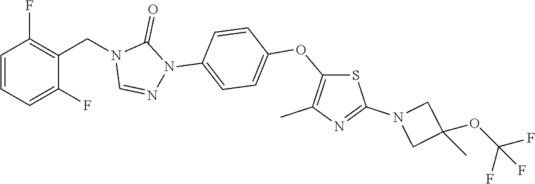 [SMILES: Cc1nc(N2CC(C)(OC(F)(F)F)C2)sc1Oc1ccc(-n2ncn(Cc3c(F)cccc3F)c2=O)cc1] | White solid, yield: 8.7% | ¹H NMR (400 MHz, DMSO-d₆) δ 8.28 (s, 1H), 7.86- 7.79 (m, 2H), 7.48 (tt, J = 8.5, 6.7 Hz, 1H), 7.22-7.07 (m, 4H), 4.97 (s, 2H), 4.15 (d, J = 9.1 Hz, 2H), 4.07 (d, J = 9.1 Hz, 2H), 2.00 (s, 3H), 1.75 (s, 3H). | 554.1 |
| | | 4-(2,6-difluorobenzyl)-2-(4-((4- | | | |
| | | methyl-2-(3-methyl-3-(trifluoro- | | | |
| | | methoxy)azetidin-1-yl)thiazol-5-yl) | | | |
| | | oxy)phenyl)-2,4-dihydro-3H-1,2,4- | | | |
| | | triazol-3-one | | | |
| Ex- am- ple 58 Meth- od 6 | ++++ | 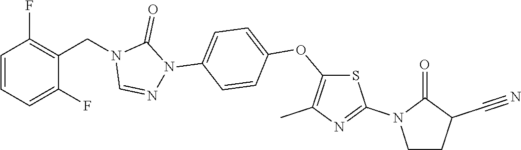 [SMILES: Cc1nc(N2CCC(C#N)C2=O)sc1Oc1ccc(-n2ncn(Cc3c(F)cccc3F)c2=O)cc1] | White solid, yield: 4.5% | ¹H NMR (400 MHz, DMSO-d₆) δ 8.29 (d, J = 5.6 Hz, 1H), 7.89-7.77 (m, 2H), 7.48 (tt, J = 8.5, 6.7 Hz, 1H), 7.22-7.06 (m, 4H), 4.97 (s, 2H), 3.65-3.11 (m, 3H), 2.96-2.85 | 509.1 |
| | | | | (m, 1H), 2.24- | |
| | | 1-(5-(4-(4-(2,6-difluorobenzyl)-5- | | 2.08 (m, 4H), | |
| | | oxo-4,5-dihydro-1H-1,2,4-triazol- | | 1.98-1.86 (m, 1H), | |
| | | 1-yl)phenoxy)-4-methylthiazol-2- | | 1.20 (d, J = 6.1 | |
| | | yl)-2-methylpyrrolidine-3- | | Hz, 3H). | |
| | | carbonitrile | | | |

### Example 59: Method 7

### 4-(2,6-difluorobenzyl)-2-(3-fluoro-4-((4-methylthiazol-5-yl)methyl)phenyl)-2,4-dihydro-3H-1,2,4-triazol-3-one

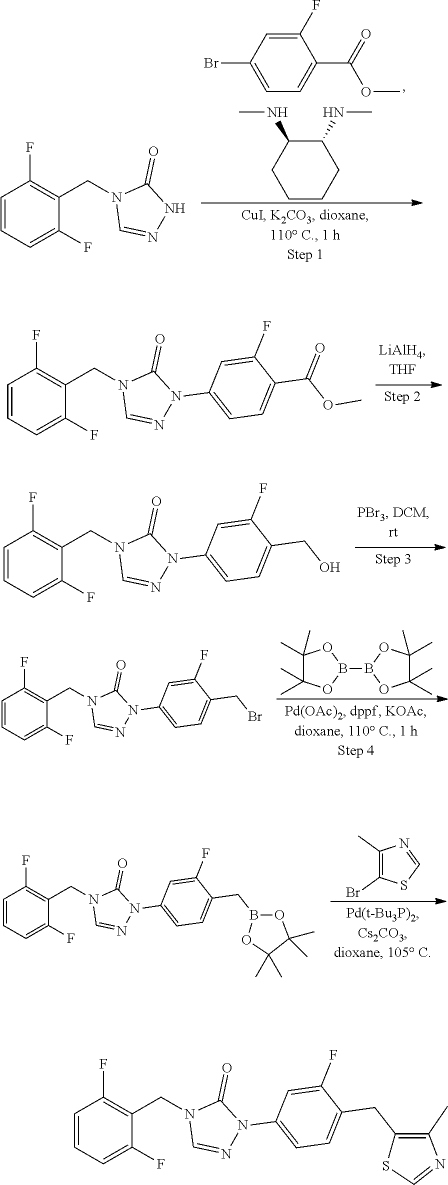

### Step 1: methyl 4-(4-(2,6-difluorobenzyl)-5-oxo-4,5-dihydro-1H-1,2,4-triazol-1-yl)-2-fluorobenzoate

A mixture solution of 4-[(2,6-difluorophenyl)methyl]-2H-1,2,4-triazol-3-one (4 g, 0.0189 mol), methyl 4-bromo-2-fluorobenzoate (5.6 g, 0.0227 mmol), Copper(I) iodide (3.6 g, 0.0189 mmol), DMDACH (8.07 g, 0.0567 mol) and K₂CO₃ (5.21 g, 0.0378 mol) in dioxane (50 mL) was stirred at 110° C. for 1 hr. After cooling, the reaction mixture was diluted with water (40 mL), extracted three times with ethyl acetate (40 mL). The organic layer were combined, and the solvent was removed under vacuum and the crude was purified through silica gel chromatography (PE/EA=1:1) to give methyl 4-(4-(2,6-difluorobenzyl)-5-oxo-4,5-dihydro-1H-1,2,4-triazol-1-yl)-2-fluorobenzoate (5 g, 66.67% yield) as a white solid. MS (m/z): 378.0 [M+H]⁺.

### Step 2: 4-(2,6-difluorobenzyl)-2-(3-fluoro-4-(hydroxymethyl)phenyl)-2,4-dihydro-3H-1,2,4-triazol-3-one

A solution of methyl 4-(4-(2,6-difluorobenzyl)-5-oxo-4,5-dihydro-1H-1,2,4-triazol-1-yl)-2-fluorobenzoate (11 g, 0.0303 mol) and LiAlH₄ (1.39 g, 0.03672 mol) in THF (20 mL) was stirred under 1 atm of N₂ atmosphere at 25° C. for 1 hr. After filtration of the reaction mixture, the filtrate was concentrated and the residue was purified through silica gel chromatography (DCM/CH₃OH=10:1) to give 4-(2,6-difluorobenzyl)-2-(3-fluoro-4-(hydroxymethyl)phenyl)-2,4-dihydro-3H-1,2,4-triazol-3-one (10.5 g, 100% yield) as an orange solid. MS (m/z): 336.1 [M+H]⁺.

### Step 3: 2-(4-(bromomethyl)-3-fluorophenyl)-4-(2,6-difluorobenzyl)-2,4-dihydro-3H-1,2,4-triazol-3-one

A mixture of 4-(2,6-difluorobenzyl)-2-(3-fluoro-4-(hydroxymethyl)phenyl)-2,4-dihydro-3H-1,2,4-triazol-3-one (10.5 g, 0.0313 mol), and PBr₃ (9.3 g, 0.0344 mol) in DCM (20 mL) was stirred at 25° C. for 2 hrs. The reaction mixture was diluted with water (40 mL), extracted three times with ethyl acetate (40 mL). The organic layer were combined, and the solvent was removed under vacuum and the crude was purified through silica gel chromatography (DCM/CH₃OH=10:1) to give 2-(4-(bromomethyl)-3-fluorophenyl)-4-(2,6-difluorobenzyl)-2,4-dihydro-3H-1,2,4-triazol-3-one (9.0 g, 72.3% yield) as white solid. MS (m/z): 398.0 [M+H]⁺.

### Step 4: 2-(4-(bromomethyl)-3-fluorophenyl)-4-(2,6-difluorobenzyl)-2,4-dihydro-3H-1,2,4-triazol-3-one

A solution of 2-[4-(bromomethyl)-3-fluorophenyl]-4-[(2,6-difluorophenyl) methyl]-1,2,4-triazol-3-one (500 mg, 1.26 mmol), 4,4,5,5-tetramethyl-2-(4,4,5,5-tetramethyl-1,3,2-dioxaborolan-2-yl)-1,3,2-dioxaborolane (637 mg, 2.51 mmol), potassium acetate (370 mg, 3.77 mmol), (acetyloxy)palladio acetate (56 mg, 0.25 mmol) and dppf (278 mg, 0.50 mmol) in dioxane (40 mL) was heated to 110° C. for 1 h under nitrogen, then cooled to rt and the solvent was removed under vacuum and the crude was purified through silica gel chromatography (DCM/MeOH=10: 1) to give 2-(4-(bromomethyl)-3-fluorophenyl)-4-(2,6-difluorobenzyl)-2,4-dihydro-3H-1,2,4-triazol-3-on e (0.2 g, 34.0% yield) as white solid. MS (m/z): 446.1 [M+H]⁺.

### Step 5: 4-(2,6-difluorobenzyl)-2-(3-fluoro-4-((4-methylthiazol-5-yl)methyl)phenyl)-2,4-dihydro-3H-1,2,4-triazol-3-one

To a solution of 4-(2,6-difluorobenzyl)-2-(3-fluoro-4-((4,4,5,5-tetramethyl-1,3,2-dioxaborolan-2-yl)methyl)phenyl)-2,4-dihydro-3H-1,2,4-triazol-3-one (1.00 eq, 100 mg, 0.225 mmol), 5-Bromo-4-methylthiazole (2.00 eq, 80 mg, 0.449 mmol) and Cs₂CO₃ (2.00 eq, 146 mg, 0.449 mmol) in 1,4-dioxane (6 mL), was added Bis(tri-tert-butylphosphine)palladium(0) (0.300 eq, 35 mg, 0.0674 mmol) at room temperature under N₂. After addition, the mixture was stirred at 85° C. for 5 h. LCMS of the reaction mixture indicated 30% product conversion. Then the reaction mixture was cooled and added water (20 mL) and extracted with EtOAc (20 mL×3). The organics were then combined and dried (Na₂SO₄) before concentration to dryness. The crude was purified with Prep-TLC (PE/EtOAc=1/1) to give the product (20 mg, 18.60% yield) as a yellow solid. MS (m/z): 417.1 [M+H]⁺. ¹H NMR (400 MHz, DMSO-d₆) δ 8.81 (s, 1H), 8.33 (s, 1H), 7.71-7.65 (m, 2H), 7.50-7.45 (m, 1H), 7.37 (t, J=8.4 Hz, 1H), 7.15 (t, J=8.1 Hz, 2H), 4.97 (s, 2H), 4.13 (s, 2H), 2.37 (s, 3H).

### Example 60: Method 7

### 2-(4-((2-(3-amino-3-(trifluoromethyl)pyrrolidin-1-yl)-4-methylthiazol-5-yl)methyl)-3-fluor ophenyl)-4-(2,6-difluorobenzyl)-2,4-dihydro-3H-1,2,4-triazol-3-one

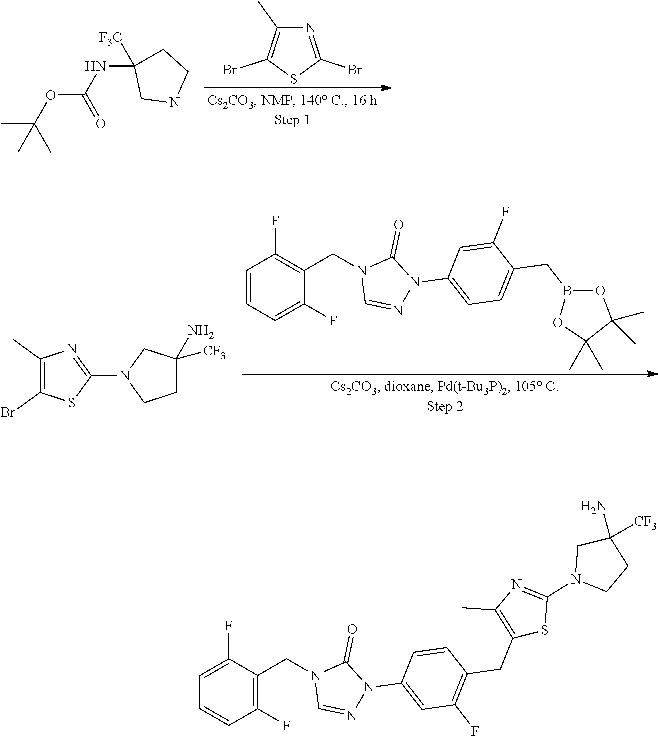

### Step 1: 1-(5-bromo-4-methylthiazol-2-yl)-3-(trifluoromethyl)pyrrolidin-3-amine

Tert-butyl (3-(trifluoromethyl)-112-pyrrolidin-3-yl)carbamate (500 mg, 1.97 mmoL),2,5-dibromo-4-methylthiazole (558 mg, 2.17 mmoL) and Cs₂CO₃ (1.284 g, 3.94 mmoL) were mixed in 10 mL NMP. Let it stir at 140° C. for 16 h. Water was added to the reaction mixture and extracted with EtOAc (30 mL×3). The organic layers were combined and evaporated to dryness and purified by C₁₈ lolumn chromatography to give 1-(5-bromo-4-methylthiazol-2-yl)-3-(trifluoromethyl)pyrrolidin-3-amine (364 mg, yield: 56%) as a brown oil. MS (m/z): 331.2 [M+H]⁺.

### Step 2: 2-(4-((2-(3-amino-3-(trifluoromethyl)pyrrolidin-1-yl)-4-methylthiazol-5-yl)methyl)-3-fluorophenyl)-4-(2,6-difluorobenzyl)-2,4-dihydro-3H-1,2,4-triazol-3-one

To a solution of 4-[(2,6-difluorophenyl)methyl]-2-[3-fluoro-4-[(4,4,5,5-tetramethyl-1,3,2-dioxaborolan-2-yl)methyl]phenyl]-1,2,4-triazol-3-one (1.20 eq, 125 mg, 0.280 mmol), Cs₂CO₃ (2.00 eq, 152 mg, 0.466 mmol), 1-(5-bromo-4-methyl-thiazol-2-yl)-3-(trifluoromethyl) pyrrolidin-3-amine (1.00 eq, 77 mg, 0.233 mmol) in 1,4-dioxane (6 mL), was added Bis(tri-tert-butylphosphine)palladium(0) (0.300 eq, 36 mg, 0.0700 mmol) at room temperature under N₂. After addition, the mixture was stirred at 110° C. for 5 h. LCMS of the reaction mixture indicated 20% product conversion. Then cooled and added water (20 mL) and extracted with EtOAc (20 mL×3). The organics were then combined and dried (Na₂SO₄) before concentration to dryness. The crude was purified with Prep-HPLC (0.1% TFA as additive) to give the product (6.0 mg, 3.77% yield) as a yellow solid. MS (m/z): 569.2 [M+H]⁺. ¹H NMR (400 MHz, DMSO-d₆) δ 8.32 (d, J=2.9 Hz, 1H), 7.72-7.61 (m, 2H), 7.57-7.41 (m, 2H), 7.33 (t, J=8.4 Hz, 1H), 7.19-7.12 (m, 2H), 4.97 (s, 2H), 3.93 (s, 2H), 3.52-3.43 (m, 2H), 3.28-3.23 (m, 2H), 2.14 (s, 3H), 2.03-1.96 (m, 2H).

Examples (Compounds) 61-67 were synthesized using a similar method to that used in Example 59.

| Com- | | | Ap- | | |
|---|---|---|---|---|---|
| pound | | | pear- | | MS |
| No. | | | ance | | (m/z) |
| Meth- | | | and | | [M + |
| od | Activity | Structure and Name | Yield | ¹H NMR Data | H]⁺ |
| Ex- am- ple 59 Meth- od 7 | ++++ | 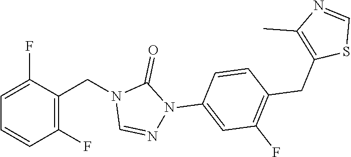 [SMILES: Cc1ncsc1Cc1ccc(-n2ncn(Cc3c(F)cccc3F)c2=O)cc1F] | yellow solid, yield: 18.60% | ¹H NMR (400 MHz, DMSO-d₆) δ 8.81 (s, 1H), 8.33 (s, 1H), 7.71-7.65 (m, 2H), 7.50-7.45 (m, 1H), 7.37 (t, J = 8.4 Hz, 1H), 7.15 (t, J = 8.1 Hz, 2H), 4.97 (s, 2H), 4.13 (s, 2H), 2.37 (s, 3H). | 417.1 |
| | | 4-(2,6-difluorobenzyl)-2-(3-fluoro- | | | |
| | | 4-((4-methylthiazol-5-yl)methyl) | | | |
| | | phenyl)-2,4-dihydro-3H-1,2,4- | | | |
| | | triazol-3-one | | | |
| Ex- | ++++ | 2-(4-((2-(3-amino-3-(trifluoromethyl) | yellow | ¹H NMR (400 MHz, DMSO-d₆) δ | 569.2 |
| am- | | pyrrolidin-1-yl)-4-methylthiazol- | solid, | 8.32 (d, J = 2.9 Hz, 1H), 7.72-7.61 | |
| ple | | 5-yl)methyl)-3-fluorophenyl)- | yield: | (m, 2H), 7.57-7.41 (m, 2H), 7.33 (t, | |
| 60 | | 4-(2,6-difluorobenzyl)-2,4- | 3.77% | J = 8.4 Hz, 1H), 7.19-7.12 (m, 2H), | |
| Meth- | | dihydro-3H-1,2,4-triazol-3- | | 4.97 (s, 2H), 3.93 (s, 2H), 3.52-3.43 | |
| od 7 | | one formate | | (m, 2H), 3.28-3.23 (m, 2H), 2.14 (s, | |
| | | | | 3H), 2.03-1.96 (m, 2H). | |
| Ex- | ++++ | (2S,3S)-1-(5-(4-(4-(2,6-difluoro- | yellow | ¹H NMR (400 MHz, DMSO-d₆) δ | 511.2 |
| am- | | benzyl)-5-oxo-4,5-dihydro-1H-1,2,4- | solid, | 10.15 (s, 1H), 8.49 (s, 1H), 8.33 (s, | |
| ple | | triazol-1-yl)-2-fluorobenzyl)-4- | yield: | 1H), 7.69-7.62 (m, 2H), 7.50-7.45 | |
| 61 | | methylthiazol-2-yl)-2- | 2.94% | (m, 1H), 7.35 (t, J = 8.5 Hz, 1H), | |
| Meth- | | methylazetidine- | | 7.19-7.12 (m, 2H), 4.97 (s, 2H), | |
| od 7 | | 3-carbonitrile formate | | 4.36-4.29 (m, 1H), 4.11-4.06 (m, | |
| | | | | 1H), 3.95 (s, 2H), 3.87 (t, J = 7.6 | |
| | | | | Hz, 1H), 3.65-3.58 (m, 1H), 2.16 (s, | |
| | | | | 3H), 1.42 (d, J = 6.0 Hz, 3H). | |
| Ex- | ++++ | 2-(4-((2-(3-amino-3-(fluoromethyl) | yellow | ¹H NMR (400 MHz, Methanol-d₄) δ | 502.1 |
| am- | | azetidin-1-yl)-4-methylthiazol-5- | solid, | 8.42 (s, 3H), 7.90 (s, 1H), 7.58 (d, J = | |
| ple | | yl)methyl)-3-fluorophenyl)-4-(2,6- | yield: | 10.1 Hz, 2H), 7.39-7.31 (m, 1H), | |
| 62 | | difluorobenzyl)-2,4-dihydro-3H- | 1.87% | 7.16 (t, J = 8.3 Hz, 1H), 6.96 (t, J = | |
| Meth- | | 1,2,4-triazol-3-one | | 8.1 Hz, 2H), 4.93 (s, 2H), 4.50-4.45 | |
| od 7 | | trifluoroacetate | | (m, 1H), 4.23-4.15 (m, 1H), 4.03 | |
| | | | | (dd, J = 9.2, 6.9 Hz, 1H), 3.85 (s, | |
| | | | | 2H), 3.70 (dd, J = 9.3, 3.7 Hz, 1H), | |
| | | | | 2.09 (s, 3H), 1.26-1.18 (m, 3H). | |
| Ex- am- ple 63 Meth- od 7 | ++++ | 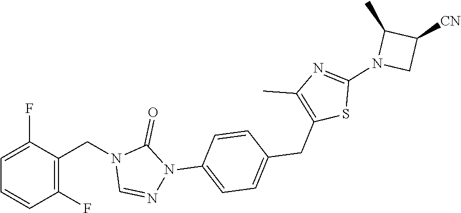 [SMILES: Cc1nc(N2C[C@H](C#N)[C@@H]2C)sc1Cc1ccc(-n2ncn(Cc3c(F)cccc3F)c2=O)cc1] | yellow solid, yield: 2.62% | ¹H NMR (400 MHz, DMSO-d₆) δ 8.27 (s, 1H), 7.80-7.74 (m, 2H), 7.49 (d, J = 6.9 Hz, 1H), 7.26 (d, J = 8.6 Hz, 2H), 7.15 (t, J = 8.1 Hz, 2H), 4.96 (s, 2H), 4.35-4.30 (m, 1H), 4.11-4.06 (m, 1H), 3.94 (s, 2H), 3.87 (t, J = 7.6 Hz, 1H), 3.64-3.58 (m, 1H), 2.16 (s, 3H), 1.42 (d, J = 6.2 Hz, 3H). | 493.2 |
| | | (2S,3S)-1-(5-(4-(4-(2,6-difluoro- | | | |
| | | benzyl)-5-oxo-4,5-dihydro-1H-1,2,4- | | | |
| | | triazol-1-yl)benzyl)-4-methyl- | | | |
| | | thiazol-2-yl)-2-methylazetidine-3- | | | |
| | | carbonitrile | | | |
| Ex- am- ple 64 Meth- od 7 | ++++ | 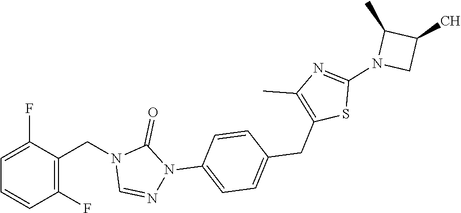 [SMILES: Cc1nc(N2C[C@H](C)[C@@H]2C)sc1Cc1ccc(-n2ncn(Cc3c(F)cccc3F)c2=O)cc1] | yellow solid, yield: 2.84% | ¹H NMR (400 MHz, DMSO-d₆) δ 8.26 (s, 1H), 7.76 (d, J = 8.6 Hz, 2H), 7.50-7.45 (m, 1H), 7.25 (d, J = 8.5 Hz, 2H), 7.15 (t, J = 8.0 Hz, 2H), 5.49 (d, J = 6.0 Hz, 1H), 4.96 (s, 2H), 4.49-4.41 (m, 1H), 4.22-4.17 (m, 1H), 4.01-3.95 (m, 1H), 3.90 (s, 2H), 3.64-3.58 (d, J = 8.4 Hz, 1H), 2.12 (s, 3H), 1.21 (d, J = 6.6 Hz, 3H). | 484.2 |
| | | 4-(2,6-difluorobenzyl)-2-(4-((2- | | | |
| | | ((2S,3S)-3-hydroxy-2-methylazetidin- | | | |
| | | 1-yl)-4-methylthiazol-5-yl)methyl) | | | |
| | | phenyl)-2,4-dihydro-3H-1,2,4- | | | |
| | | triazol-3-one | | | |
| Ex- | ++++ | 4-benzyl-2-(4-benzylphenyl)-5- | | NMR (400 MHz, CDCl₃) δ 7.90- | 355.8 |
| am- | | methyl-2,4-dihydro-3H-1,2,4- | | 7.85 (m, 2H), 7.38-7.27 (m, 8H), | |
| ple | | triazol-3-one | | 7.24-7.15 (m, 4H), 4.87 (s, 2H), | |
| 65 | | | | 3.98 (s, 2H), 2.18 (s, 3H). | |
| Meth- | | | | | |
| od 7 | | | | | |
| Ex- am- ple 66 Meth- od 7 | ++++ | 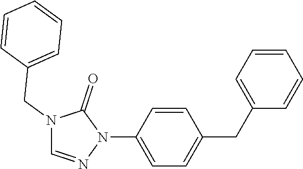 [SMILES: O=c1n(Cc2ccccc2)cnn1-c1ccc(Cc2ccccc2)cc1] | | ¹H NMR (400 MHz, CDCl₃) δ 7.90- 7.85 (m, 2H), 7.43 (s, 1 H), 7.41- 7.26 (m, 8H), 7.25-7.16 (m, 4H), 4.86 (s, 2H), 3.99 (s, 2H). | 341.8 |
| | | 4-benzyl-2-(4-benzylphenyl)-2,4- | | | |
| | | dihydro-3H-1,2,4-triazol-3-one | | | |
| Ex- am- ple 67 Meth- od 7 | +++ | 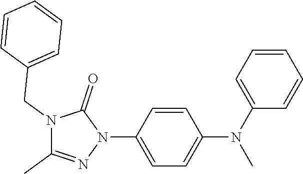 [SMILES: Cc1nn(-c2ccc(N(C)c3ccccc3)cc2)c(=O)n1Cc1ccccc1] | | ¹H NMR (400 MHz, CDCl₃) δ 7.89- 7.81 (m, 2H), 7.39-7.27 (m, 7H), 7.13 (d, J = 8.8 Hz, 2H), 7.04 (d, J = 7.6 Hz, 2H), 6.96 (t, J = 7.2 Hz, 1H), 4.89 (s, 2H), 3.33 (s, 3H), 2.20 (s, 3H). | 370.8 |
| | | 4-benzyl-5-methyl-2-(4-(methyl | | | |
| | | (phenyl)amino)phenyl)-2,4-dihydro- | | | |
| | | 3H-1,2,4-triazol-3-one | | | |

### Example 68: Method 8

### 2-(4-((2-(3-(2-aminopropan-2-yl)azetidin-1-yl)-4-methylthiazol-5-yl)oxy)phenyl)-4-(2,6-dif luorobenzyl)-2,4-dihydro-3H-1,2,4-triazol-3-one

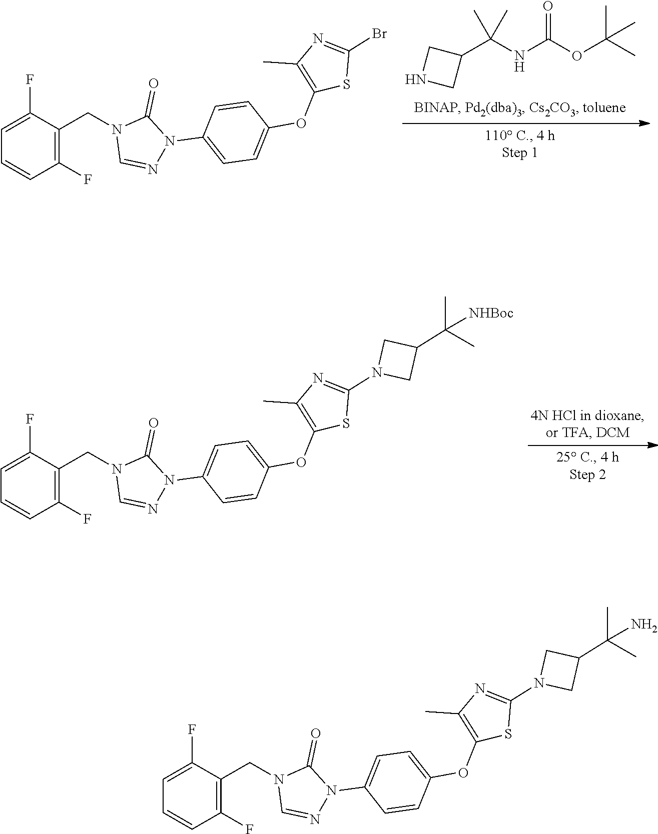 [SMILES: Cc1nc(Br)sc1Oc1ccc(-n2ncn(Cc3c(F)cccc3F)c2=O)cc1 ; CC(C)(C)OC(=O)NC(C)(C)C1CNC1 ; Cc1nc(N2CC(C(C)(C)NC(=O)OC(C)(C)C)C2)sc1Oc1ccc(-n2ncn(Cc3c(F)cccc3F)c2=O)cc1 ; Cc1nc(N2CC(C(C)(C)N)C2)sc1Oc1ccc(-n2ncn(Cc3c(F)cccc3F)c2=O)cc1]

### Step 1

### tert-butyl (2-(1-(5-(4-(4-(2,6-difluorobenzyl)-5-oxo-4,5-dihydro-1H-1,2,4-triazol-1-yl) phenoxy)-4-methylthiazol-2-yl)azetidin-3-yl)propan-2-yl)carbamate

To a stirred solution of 2-(4-((2-bromo-4-methylthiazol-5-yl)oxy)phenyl)-4-(2,6-difluorobenzyl)-2,4-dihydro-3H-1,2,4-triazol-3-one (50 mg, 0.1 mmol) and tert-butyl (2-(azetidin-3-yl)propan-2-yl)carbamate (38 mg, 0.15 mmol) in toluene (1 mL) were added BINAP (12 mg, 0.02 mmol), Pd₂(dba)₃ (9 mg, 0.01 mmol), and Cs₂CO₃ (100 mg, 0.3 mmol). The resulted mixture was stirred for additional 2 h at 100° C. under N₂. The mixture was allowed to cool down to rt. The resulted mixture was diluted with water (10 mL). The resulted mixture was extracted with EtOAc (3×10 mL). The combined organic layers were washed with brine (10 mL), dried over anhydrous Na₂SO₄, after filtration, the filtrate was concentrated under reduced pressure. The residue was purified by silica gel column chromatography, eluted with (PE: EtOAc=1:1) to afford tert-butyl (2-(1-(5-(4-(4-(2,6-difluorobenzyl)-5-oxo-4,5-dihydro-1H-1,2,4-triazol-1-yl)phenoxy)-4-methylthiazol-2-yl)azetidin-3-yl)propan-2-yl)carbamate (20 mg, 36% yield) as a white solid. MS (m/z): 613.2 [M+H]⁺. ¹H NMR (400 MHz, MeOD) δ 7.88 (s, 1H), 7.77-7.66 (m, 2H), 7.40-7.29 (m, 1H), 7.00-6.90 (m, 4H), 4.93 (s, 2H), 3.94-3.75 (m, 4H), 2.15-2.02 (m, 1H), 1.93 (s, 3H), 1.31 (s, 9H), 1.26-1.20 (m, 6H).

### Step 2: 2-(4-((2-(3-(2-aminopropan-2-yl)azetidin-1-yl)-4-methylthiazol-5-yl)oxy)phenyl)-4-(2,6-difluorobenzyl)-2,4-dihydro-3H-1,2,4-triazol-3-one

To a stirred solution of tert-butyl (2-(1-(5-(4-(4-(2,6-difluorobenzyl)-5-oxo-4,5-dihydro-1H-1,2,4-triazol-1-yl)phenoxy)-4-methylthiazol-2-yl)azetidin-3-yl)propan-2-yl)carbamate (20 mg, 0.03 mmol) in DCM (5 mL) was added 4N HCl in dioxane or TFA (0.5 mL) at rt. The resulted mixture was stirred for additional 2 h at 25° C. The resulted mixture was concentrated under reduced pressure. The residue was purified by reverse phase flash chromatography with the following conditions: column: Spherical C₁₈ 40-60 um, 40 g; Mobile phase B: ACN; Flow rate: 40 mL/min; Gradient: 35% B-60% B in 20 min; Detector: 254 nm. The fractions containing the desired product were concentrated under reduced pressure to afford the titled compound 2-(4-((2-(3-(2-aminopropan-2-yl)azetidin-1-yl)-4-methylthiazol-5-yl)oxy)phenyl)-4-(2,6-difluorobenzyl)-2,4-dihydro-3H-1,2,4-triazol-3-one (3.4 mg, 21% yield) as an off-white solid. LC-MS (m/z): 513.2 [M+H]⁺. ¹H NMR (400 MHz, MeOD) δ 7.91 (s, 1H), 7.83 (d, J=5.2 Hz, 2H), 7.42-7.28 (m, 1H), 7.20-6.90 (m, 4H), 4.94 (s, 2H), 4.43-4.19 (m, 2H), 3.69-3.45 (m, 2H), 2.25-1.90 (m, 4H), 1.57-1.25 (m, 6H).

Examples (Compounds) 69-73 were synthesized using a similar method to that used in Example 68.

| Com- | | | Ap- | | |
|---|---|---|---|---|---|
| pound | | | pear- | | MS |
| No. | | | ance | | (m/z) |
| Meth- | Ac- | | and | | [M + |
| od | tivity | Structure and Name | Yield | ¹H NMR Data | H]⁺ |
| Ex- am- ple 68 Meth- od 8 | ++++ | 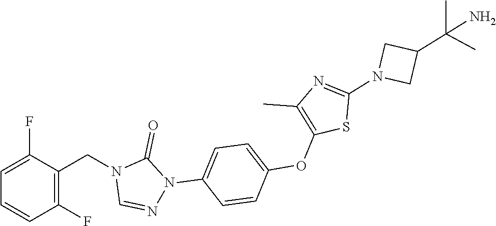 [SMILES: Cc1nc(N2CC(C(C)(C)N)C2)sc1Oc1ccc(-n2ncn(Cc3c(F)cccc3F)c2=O)cc1] | off- white solid, yield: 21% | ¹H NMR (400 MHz, MeOD) δ 7.91 (s, 1H), 7.83 (d, J = 5.2 Hz, 2H), 7.42-7.28 (m, 1H), 7.20-6.90 (m, 4H), 4.94 (s, 2H), 4.43-4.19 (m, 2H), 3.69-3.45 (m, 2H), 2.25-1.90 (m, 4H), 1.57-1.25 (m, 6H). | 513.2 |
| | | 2-(4-((2-(3-(2-aminopropan-2-yl) | | | |
| | | azetidin-1-yl)-4-methylthiazol-5- | | | |
| | | yl)oxy)phenyl)-4-(2,6-difluoro- | | | |
| | | benzyl)-2,4-dihydro-3H-1,2,4- | | | |
| | | triazol-3-one | | | |
| Ex- am- ple 69 Meth- od 8 | ++++ | 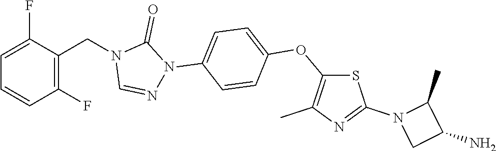 [SMILES: Cc1nc(N2C[C@@H](N)[C@@H]2C)sc1Oc1ccc(-n2ncn(Cc3c(F)cccc3F)c2=O)cc1] | white solid, yield: 24% | ¹H NMR (400 MHz, MeOD) δ 8.40 (s, 2H), 7.89 (s, 1H), 7.73 (d, J = 9.1 Hz, 2H), 7.42-7.28 (m, 1H), 7.01- 6.90 (m, 4H), 4.94 (s, 2H), 4.13 (t, J = 7.9 Hz, 1H), 4.02-3.87 (m, 1H), 3.66-3.43 (m, 2H), 1.95 (s, 3H), 1.42-1.35 (m, 3H). | 485.2 |
| | | 2-(4-((2-((2S,3R)-3-amino-2-methyl- | | | |
| | | azetidin-1-yl)-4-methylthiazol- | | | |
| | | 5-yl)oxy)phenyl)-4-(2,6-difluoro- | | | |
| | | benzyl)-2,4-dihydro-3H-1,2,4- | | | |
| | | triazol-3-one | | | |
| Ex- am- ple 70 Meth- od 8 | ++++ | 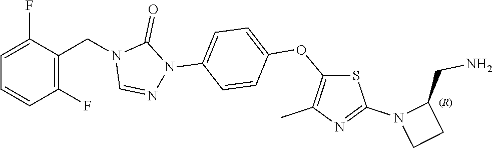 | white solid, yield: | ¹H NMR (400 MHz, MeOD) δ 8.01 (s, 1H), 7.92 (s, 2H), 7.51-7.40 (m, 1H), 7.30-7.00 (m, 4H), 5.05 (s, 2H), 4.50-4.20 (m, 2H), 3.80-3.41 (m, 3H), 2.95-2.55 (m, 2H), 2.22 (s, 3H). | 485.1 |
| | | (R)-2-(4-((2-(2-(aminomethyl) | | | |
| | | azetidin-1-yl)-4-methylthiazol-5-yl) | | | |
| | | oxy)phenyl)-4-(2,6-difluorobenzyl)- | | | |
| | | 2,4-dihydro-3H-1,2,4-triazol-3- | | | |
| | | one | | | |
| Ex- am- ple 71 Meth- od 8 | ++++ | 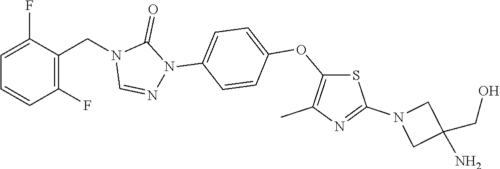 [SMILES: Cc1nc(N2CC(N)(CO)C2)sc1Oc1ccc(-n2ncn(Cc3c(F)cccc3F)c2=O)cc1] | off- white solid, yield: | ¹H NMR (400 MHz, MeOD) δ 7.90 (s, 1H), 7.82 (s, 2H), 7.34 (dd, J = 13.9, 7.0 Hz, 1H), 7.20-6.95 (m, 4H), 4.94 (s, 2H), 4.41 (s, 3H), 3.87 (s, 2H), 3.70-3.51 (m, 2H), 2.11 (s, 3H). | 501.1 |
| | | 2-(4-((2-(3-amino-3-(hydroxymethyl) | | | |
| | | azetidin-1-yl)-4-methylthiazol- | | | |
| | | 5-yl)oxy)phenyl)-4-(2,6-difluoro | | | |
| | | benzyl)-2,4-dihydro-3H-1,2,4- | | | |
| | | triazol-3-one | | | |
| Ex- am- ple 72 Meth- od 8 | ++++ | 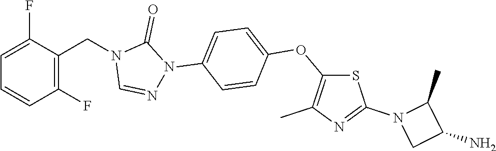 [SMILES: Cc1nc(N2C[C@@H](N)[C@@H]2C)sc1Oc1ccc(-n2ncn(Cc3c(F)cccc3F)c2=O)cc1] | white solid, yield: 55.6% | ¹H NMR (400 MHz, MeOD) δ 7.87-7.81 (m, 2H), 7.54 (s, 1H), 7.34 (tt, J = 8.4, 6.5 Hz, 1H), 7.04- 7.00 (m, 2H), 6.99-6.93 (m, 2H), 4.95 (s, 2H), 4.23 (t, J = 7.6 Hz, 1H), 4.00-3.87 (m, 1H), 3.66- 3.49 (m, 2H), 2.07-2.03 (m, 3H), 1.47 (d, J = 6.3 Hz, 3H). | 485.2 |
| | | 2-(4-((2-((2S,3R)-3-amino-2-methyl- | | | |
| | | azetidin-1-yl)-4-methylthiazol- | | | |
| | | 5-yl)oxy)phenyl)-4-(2,6-difluoro- | | | |
| | | benzyl)-2,4-dihydro-3H-1,2,4- | | | |
| | | triazol-3-one | | | |
| Ex- am- ple 73 Meth- od 8 | ++++ | 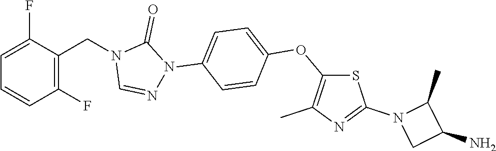 [SMILES: Cc1nc(N2C[C@H](N)[C@@H]2C)sc1Oc1ccc(-n2ncn(Cc3c(F)cccc3F)c2=O)cc1] | white solid, yield: | ¹H NMR (400 MHz, DMSO-d₆) δ 8.28 (s, 1H), 7.85-7.78 (m, 2H), 7.52-7.44 (m, 1H), 7.21-7.07 (m, 4H), 4.97 (s, 2H), 4.34 (p, J = 7.2 Hz, 1H), 4.14-4.03 (m, 1H), 3.91-3.86 (m, 1H), 3.65-3.60 (m, 1H), 1.98 (s, 3H), 1.31 (d, J = 6.8 Hz, 3H). | 485.2 |
| | | 2-(4-((2-((2S,3S)-3-amino-2-methyl- | | | |
| | | azetidin-1-yl)-4-methylthiazol-5- | | | |
| | | yl)oxy)phenyl)-4-(2,6-difluoro- | | | |
| | | benzyl)-2,4-dihydro-3H-1,2,4- | | | |
| | | triazol-3-one | | | |

### Example 74: Method 9

### 4-(2,6-difluorobenzyl)-2-(4-((2-(3-ethoxyazetidin-1-yl)-4-methylthiazol-5-yl)oxy)phenyl)-2,4-dihydro-3H-1,2,4-triazol-3-one

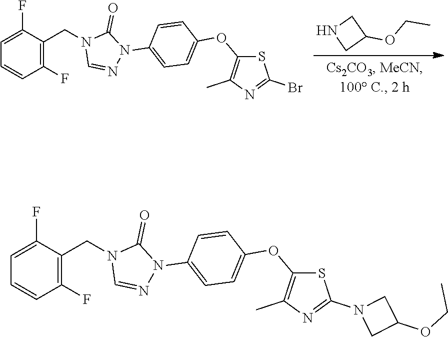

To a stirred solution of 2-(4-((2-bromo-4-methylthiazol-5-yl)oxy)phenyl)-4-(2,6-difluorobenzyl)-2,4-dihydro-3H-1,2,4-triazol-3-one (100 mg, 0.21 mmol), 73-ethoxyazetidine (32 mg, 0.32 mmol) and Cs₂CO₃ (102 mg, 0.32 mmol) in MeCN (5 mL) were added. The reaction mixture was stirred at 100° C. for 2 h. The resulted mixture was diluted with EtOAc (10 mL), the solution was successively washed with water (2×5 mL) and brine (5 mL), dried over anhydrous Na₂SO₄, and after filtration, the filtrate was concentrated under reduced pressure. The residue was purified by TLC, layered with (DCM:MeOH) (50:1) to afford the titled compound (32 mg, 30.5% yield) as a white solid. MS (m/z): 500.2 [M+H]⁺. ¹H NMR (400 MHz, CDCl₃) δ 7.92-7.83 (m, 2H), 7.55 (s, 1H), 7.42-7.29 (m, 1H), 7.07-6.94 (m, 4H), 4.97 (s, 2H), 4.54-4.40 (m, 1H), 4.3-4.21 (m, 2H), 4.04-3.93 (s, 2H), 3.48 (q, J=7.2 Hz, 2H), 2.11 (s, 3H), 1.23 (t, J=6.8 Hz, 3H).

Example (Compound) 75 was synthesized using a similar method to that used in Example 74.

| Com- | | | Ap- | | |
|---|---|---|---|---|---|
| pound | | | pear- | | MS |
| No. | | | ance | | (m/z) |
| Meth- | Ac- | | and | | [M + |
| od | tivity | Structure and Name | Yield | ¹H NMR Data | H]⁺ |
| Ex- am- ple 74 Meth- od 9 | ++++ | 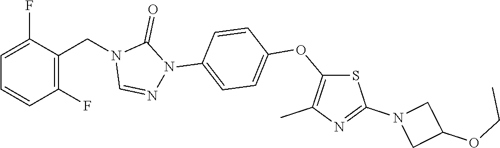 [SMILES: CCOC1CN(c2nc(C)c(Oc3ccc(-n4ncn(Cc5c(F)cccc5F)c4=O)cc3)s2)C1] | white solid, yield: 30.5% | ¹H NMR (400 MHz, CDCl₃) δ 7.92-7.83 (m, 2H), 7.55 (s, 1H), 7.42-7.29 (m, 1H), 7.07-6.94 (m, 4H), 4.97 (s, 2H), 4.54-4.40 (m, 1H), 4.3-4.21 (m, 2H), 4.04-3.93 (s, 2H), 3.48 (q, J = 7.2 Hz, 2H), 2.11 (s, 3H), 1.23 (t, J = 6.8 Hz, 3H). | 500.2 |
| | | 4-(2,6-difluorobenzyl)-2-(4-((2-(3- | | | |
| | | ethoxyazetidin-1-yl)-4-methyl- | | | |
| | | thiazol-5-yl)oxy)phenyl)-2,4- | | | |
| | | dihydro-3H-1,2,4-triazol-3-one | | | |
| Ex- am- ple 75 Meth- od 9 | ++++ | 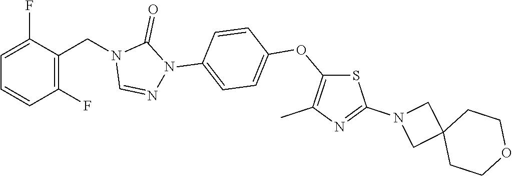 [SMILES: Cc1nc(N2CC3(CCOCC3)C2)sc1Oc1ccc(-n2ncn(Cc3c(F)cccc3F)c2=O)cc1] | white solid, yield: 15.8% | NA | 526.1 |
| | | 4-(2,6-difluorobenzyl)-2-(4-((4- | | | |
| | | methyl-2-(7-oxa-2-azaspiro[3.5] | | | |
| | | nonan-2-yl)thiazol-5-yl)oxy)phenyl)- | | | |
| | | 2,4-dihydro-3H-1,2,4-triazol-3- | | | |
| | | one | | | |

### Example 76: Method 10

### (S)-4-(2,6-difluorobenzyl)-2-(3-fluoro-4-((2-(4-hydroxy-2-oxopyrrolidin-1-yl)-4-methylthiazol-5-yl)oxy)phenyl)-2,4-dihydro-3H-1,2,4-triazol- 3-one

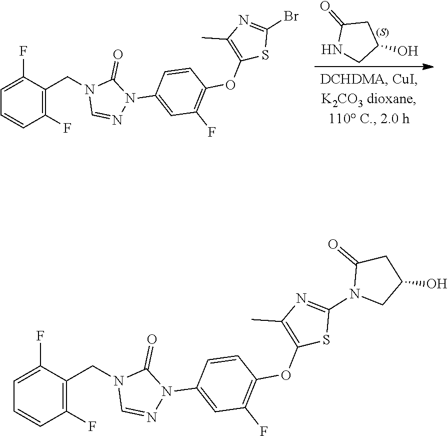

To a stirred solution of 2-(4-((2-bromo-4-methylthiazol-5-yl)oxy)-3-fluorophenyl)-4-(2,6-difluorobenzyl)-2,4-dihydro-3H-1,2,4-triazol-3-one (50 mg, 0.10 mmol) and (S)-4-hydroxypyrrolidin-2-one (10 mg, 0.10 mmol) in dioxane (10 mL) were added CuI (10 mg, 0.05 mmol), (1R,2R)—N,N′-Dimethyl-1,2-cyclohexanediamine (7 mg, 0.05 mmol) K₂CO₃ (42 mg, 0.03 mmol) at rt. The resulted mixture was stirred for additional 2 h at 110° C. under Ar. The mixture was allowed to cool down to rt. The resulted mixture was diluted with water (30 mL). The resulted mixture was extracted with EtOAc (3×15 mL). The combined Organic layers were washed with brine (20 mL), dried over anhydrous Na₂SO₄, after filtration, the filtrate was concentrated under reduced pressure. The residue was purified by silica gel column chromatography, eluted with (PE: EtOAc=1:1) to afford the titled compound (30 mg, 57.7% yield) as an off-white solid. LC-MS (m/z): 518.0 [M+H]⁺. ¹H NMR (400 MHz, DMSO) δ 8.33 (s, 1H), 7.86 (dd, J=12.6, 2.4 Hz, 1H), 7.65 (d, J=9.2 Hz, 1H), 7.51-7.43 (m, 1H), 7.21-7.08 (m, 3H), 5.45 (d, J=3.8 Hz, 1H), 4.97 (s, 2H), 4.53-4.41 (m, 1H), 4.07 (dd, J=11.4, 4.9 Hz, 1H), 3.91-3.83 (m, 1H), 2.95 (dd, J=17.4, 5.9 Hz, 1H), 2.38 (d, J=17.5 Hz, 1H), 2.15 (s, 3H).

| Com- | | | Ap- | | |
|---|---|---|---|---|---|
| pound | | | pear- | | MS |
| No. | | | ance | | (m/z) |
| Meth- | | | and | | [M + |
| od | Activity | Structure and Name | Yield | ¹H NMR Data | H]⁺ |
| Ex- am- ple 76 Meth- od 10 | ++++ | 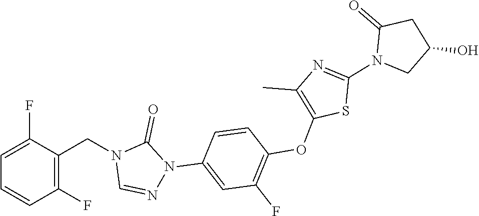 [SMILES: Cc1nc(N2C[C@@H](O)CC2=O)sc1Oc1ccc(-n2ncn(Cc3c(F)cccc3F)c2=O)cc1F] | off- white solid, yield: 57.7% | ¹H NMR (400 MHz, DMSO-d₆) δ 8.33 (s, 1H), 7.86 (dd, J = 12.6, 2.4 Hz, 1H), 7.65 (d, J = 9.2 Hz, 1H), 7.51-7.43 (m, 1H), 7.21-7.08 (m, 3H), 5.45 (d, J = 3.8 Hz, 1H), 4.97 (s, 2H), 4.53-4.41 (m, 1H), 4.07 (dd, J = 11.4, 4.9 Hz, 1H), 3.91-3.83 (m, 1H), 2.95 (dd, J = 17.4, 5.9 Hz, 1H), 2.38 (d, J = 17.5 Hz, 1H), 2.15 (s, 3H). | 518.0 |
| | | (S)-4-(2,6-difluorobenzyl)-2-(3- | | | |
| | | fluoro-4-((2-(4-hydroxy-2-oxo- | | | |
| | | pyrrolidin-1-yl)-4-methylthiazol- | | | |
| | | 5-yl)oxy)phenyl)-2,4-dihydro- | | | |
| | | 3H-1,2,4-triazol-3-one | | | |

### Example 77: Method 11

### 2-(4-(4-(2,6-difluorobenzyl)-5-oxo-4,5-dihydro-1H-1,2,4-triazol-1-yl)phenoxy)thiazole-5-carbonitrile

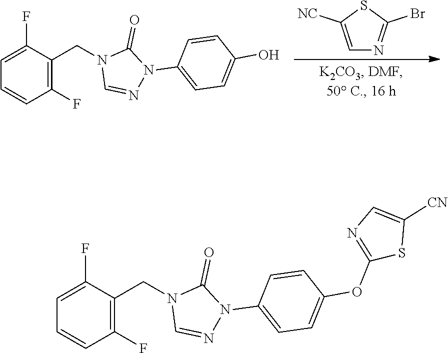 [SMILES: O=c1n(Cc2c(F)cccc2F)cnn1-c1ccc(O)cc1 ; N#Cc1cnc(Br)s1 ; N#Cc1cnc(Oc2ccc(-n3ncn(Cc4c(F)cccc4F)c3=O)cc2)s1]

A solution of 4-[(2,6-difluorophenyl)methyl]-2-(4-hydroxyphenyl)-1,2,4-triazol-3-one (50 mg, 0.16 mmol), 2-bromo-1,3-thiazole-5-carbonitrile (47 mg, 0.24 mmol), K₂CO₃ (46 mg, 0.33 mmol) in DMSO (2 mL) was stirred at 50° C. for 16 h. The resulted mixture was diluted with water (20 mL). The resulted mixture was extracted with EtOAc (3×10 mL). The combined organic layers were washed with brine (20 mL), dried over anhydrous Na₂SO₄, and after filtration, the filtrate was concentrated under reduced pressure. The residue was purified by Prep-TLC (EtOAc:PE=1:1) to afford 2-(4-(4-(2,6-difluorobenzyl)-5-oxo-4,5-dihydro-1H-1,2,4-triazol-1-yl)phenoxy)thiazole-5-carb onitrile (21.1 mg, 28.8% yield) as a white solid. LC-MS (m/z): 412.0 [M+H]⁺. ¹H NMR (400 MHz, CDCl₃) δ 8.15-8.08 (m, 1H), 7.76 (s, 1H), 7.61 (s, 1H), 7.42-7.31 (m, 2H), 7.03-6.94 (m, 4H), 4.99 (s, 2H).

Examples (Compounds) 78-79 were synthesized using a similar method to that used in Example 77.

| Com- | | | | | |
|---|---|---|---|---|---|
| pound | | | | | |
| No. | | | | | MS |
| Meth- | | | Appearance | | (m/z) |
| od | Activity | Structure and Name | and Yield | ¹H NMR Data | [M + H]⁺ |
| Ex- am- ple 77 Meth- od 11 | +++ | 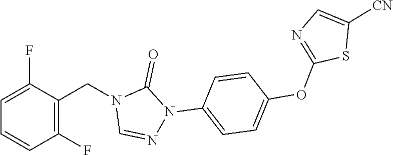 [SMILES: N#Cc1cnc(Oc2ccc(-n3ncn(Cc4c(F)cccc4F)c3=O)cc2)s1] | white solid, yield:28.8% | 1H NMR (400 MHz, CDCl₃) δ 8.15- 8.08 (m, 1H), 7.76 (s, 1H), 7.61 (s, 1H), 7.42-7.31 (m, 2H), 7.03- 6.94 (m, 4H), 4.99 (s, 2H). | 412.0 |
| | | 2-(4-(4-(2,6-difluorobenzyl)-5- | | | |
| | | oxo-4,5-dihydro-1H-1,2,4-triazol- | | | |
| | | 1-yl)phenoxy)thiazole-5- | | | |
| | | carbonitrile | | | |
| Ex- am- ple 78 Meth- od 11 | +++ | 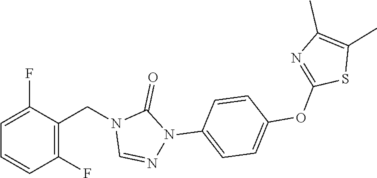 [SMILES: Cc1nc(Oc2ccc(-n3ncn(Cc4c(F)cccc4F)c3=O)cc2)sc1C] | white solid, yield: | ¹H NMR (400 MHz, DMSO-d₆) δ 8.32 (s, 1H), 7.94-7.87 (m, 2H), 7.53-7.43 (m, 1H), 7.41-7.34 (m, 2H), 7.21-7.12 (m, 2H), 4.97 (s, 2H), 2.22 (d, J = 0.7 Hz, 3H), 2.09 (d, J = 0.8 Hz, 3H). | 415.0 |
| | | 4-(2,6-difluorobenzyl)-2-(4-((4,5- | | | |
| | | dimethylthiazol-2-yl)oxy)phenyl)- | | | |
| | | 2,4-dihydro-3H-1,2,4-triazol-3- | | | |
| | | one | | | |
| Ex- am- ple79 Meth- od 11 | ++++ | 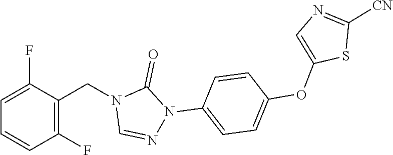 [SMILES: N#Cc1ncc(Oc2ccc(-n3ncn(Cc4c(F)cccc4F)c3=O)cc2)s1] | white solid, yield: | ¹H NMR (400 MHz, DMSO-d₆) δ 8.34 (s, 1H), 7.96 (s, 1H), 7.95- 7.90 (m, 2H), 7.54-7.46 (m, 1H), 7.46-7.41 (m, 2H), 7.17 (t, J = 8.1 Hz, 2H), 4.98 (s, 2H). | 412.0 |
| | | 5-(4-(4-(2,6-difluorobenzyl)-5- | | | |
| | | oxo-4,5-dihydro-1H-1,2,4-triazol- | | | |
| | | 1-yl)phenoxy)thiazole-2- | | | |
| | | carbonitrile | | | |

### Example 80: Method 12

### 5-(2-fluoro-4-(7-fluoro-3-oxo-5-phenyl-3,5,6,7-tetrahydro-2H-pyrrolo[2,1-c][1,2,4]triazol-2-yl)phenoxy)-4-methylthiazole-2-carboxamide

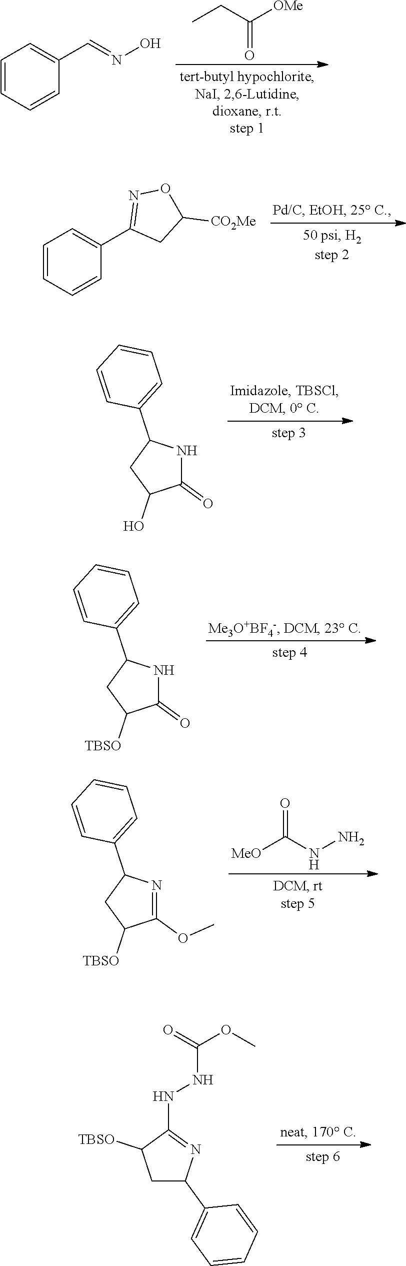

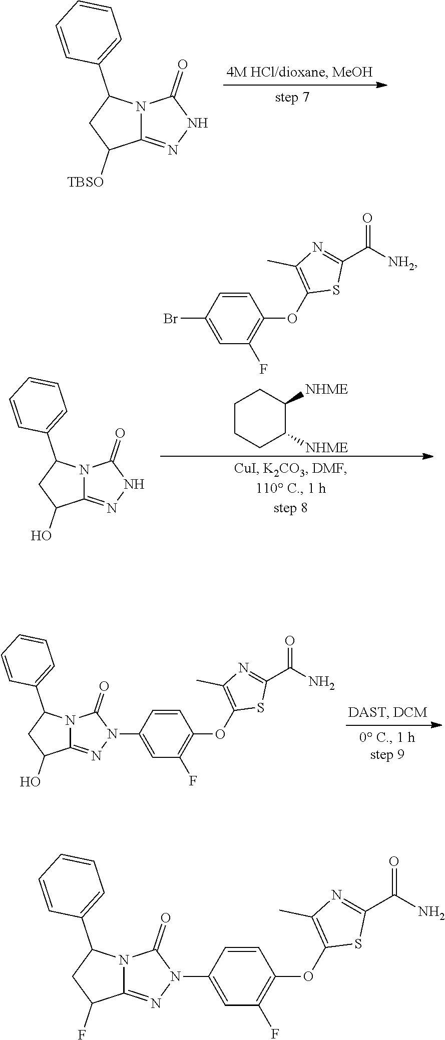

### Step 1: methyl 3-phenyl-4,5-dihydroisoxazole-5-carboxylate

To a stirred solution of benzaldehyde oxime (120 g, 0.99 mol) in 1,4-dioxane (1200 mL) was added methyl prop-2-enoate (94 g, 1.09 mol), NaI (164 g, 1.09 mmol), 2,6-lutidine (117 g, 1.09 mol) and tert-butyl hypochlorite (118 g, 1.09 mmol) batchwise at 25° C. The resulted mixture was stirred for additional 18 h at 25° C. The resulted mixture was quenched with water (1000 mL). The resulted mixture was extracted with EtOAc (3×800 mL). The combined organic layers were washed with brine (500 mL), dried over anhydrous Na₂SO₄, filtered, and the filtrate was concentrated under reduced pressure. The residue was purified by silica gel, eluted with (PE:EtOAc) (10:1) to afford crude methyl 3-phenyl-4,5-dihydroisoxazole-5-carboxylate (140 g, 65.4% yield) as a yellow solid. MS (m/z): 206.0 [M+H]⁺.

### Step 2: ethyl 5-bromo-4-methylisoxazole-3-carboxylate

To a solution of methyl 3-phenyl-4,5-dihydroisoxazole-5-carboxylate (46 g, 224 mmol) in EtOH (1000 mL) was added Pd/C (23 g) at 25° C. The mixture was stirred at 25° C. under H₂ 50 psi for 5 hours. The reaction solution was filtered, and the filtrate was concentrated in vacuo to give crude 3-hydroxy-5-phenylpyrrolidin-2-one (48 g crude) 74% purity in LCMS as a yellow oil. LC-MS (m/z) 178.0[M+H]⁺.

### Step 3: 3-((tert-butyldimethylsilyl)oxy)-5-phenylpyrrolidin-2-one

To a mixture of 3-hydroxy-5-phenylpyrrolidin-2-one (75 g, 423.2 mmol) in DCM (750 mL) was added imidazole (57.5 g, 0.85 mol) and TBSCl (95.5 g, 0.64 mmol) batchwise at rt, the reaction mixture was stirred at 30° C. for 18 h. The mixture was allowed to cool down to rt. The resulted mixture was diluted with water (300 mL). The resulted mixture was extracted with DCM (3×500 mL). The combined organic layers were washed with brine (800 mL), dried over anhydrous Na₂SO₄, and after filtration, the filtrate was concentrated under reduced pressure. The residue was purified by silica gel, eluted with (PE: EtOAc) (10:1) to afford 3-((tert-butyldimethylsilyl)oxy)-5-phenylpyrrolidin-2-one (86 g, 69.9% yield) as a yellow solid. LC-MS (m/z) 292.1[M+H]⁺.

### Step 4: 4-((tert-butyldimethylsilyl)oxy)-5-methoxy-2-phenyl-3,4-dihydro-2H-pyrrole

To a stirred solution of 3-((tert-butyldimethylsilyl)oxy)-5-phenylpyrrolidin-2-one (86 g, 291 mmol) in DCM (800 mL) was added trimethyloxonium tetrafluoroborate (47.5 g, 320 mmol). The reaction mixture was stirred at 25° C. for 18 h. The reaction solution was used directly in the next step.

### Step 5: methyl 2-(4-((tert-butyldimethylsilyl)oxy)-2-phenyl-3,4-dihydro-2H-pyrrol-5-yl) hydrazine-1-carboxylate

To a solution of 4-((tert-butyldimethylsilyl)oxy)-5-methoxy-2-phenyl-3,4-dihydro-2H-pyrrole in DCM (750 mL) was added a solution of methoxycarbohydrazide (29.5 g, 320 mmol) in DCM (200 mL) dropwise at 25° C. under N₂, and the mixture was stirred at 25° C. for 6 h, the mixture was quenched with ice-water. The mixture was extracted with DCM (100 mL×3). The combined organic layers were washed with water (500 mL×2), brine (500 mL), dried over Na₂SO₄ and concentrated. The residue was purified by silica gel, eluted with (DCM:MeOH) (30:1) to afford methyl 2-(4-((tert-butyldimethylsilyl)oxy)-2-phenyl-3,4-dihydro-2H-pyrrol-5-yl)hydrazine-1 carboxylate (67 g, 62.6% yield of 2 steps) as a yellow solid. LC-MS (m/z): 364.2 [M+H]⁺.

### Step 6: methyl 2-(4-((tert-butyldimethylsilyl)oxy)-2-phenyl-3,4-dihydro-2H-pyrrol-5-yl) hydrazine-1-carboxylate

Methyl 2-(4-((tert-butyldimethylsilyl)oxy)-2-phenyl-3,4-dihydro-2H-pyrrol-5-yl)hydrazine-1-carboxylate (30 g, 82.5 mmol) in a single-neck bottle was heated at 170° C. for 4 hours without solvent under N₂. The mixture was cooled to r.t to afford crude 7-((tert-butyldimethylsilyl)oxy)-5-phenyl-2,5,6,7-tetrahydro-3H-pyrrolo[2,1-c][1,2,4]triazol-3-one (27 g crude) 88% purity in LCMS as a yellow solid. LC-MS (m/z): 332.1 [M+H]⁺.

### Step 7

### 7-hydroxy-5-phenyl-2,5,6,7-tetrahydro-3H-pyrrolo[2,1-c][1,2,4]triazol-3-one

To a stirred solution of 7-((tert-butyldimethylsilyl)oxy)-5-phenyl-2,5,6,7-tetrahydro-3H-pyrrolo[2,1-c][1,2,4]triazol-3-one (27 g, 81.5 mmol) in MeOH (270 mL) was added 4M HCl in dioxane (30 mL) at 25° C. The reaction mixture was stirred at 25° C. for 3 hours. The mixture was treated with sat. Na₂CO₃ solution to adjust to pH=9 and concentrated. The residue was purified by silica gel, eluted with (DCM:MeOH) (20:1) to afford 7-hydroxy-5-phenyl-2,5,6,7-tetrahydro-3H-pyrrolo[2,1-c][1,2,4]triazol-3-one (4 g, 21.8% yield) as a yellow solid. LC-MS (m/z): 218.1 [M+H]⁺. ¹H NMR (400 MHz, DMSO_d6) δ 11.47 (d, J=11.6 Hz, 1H), 7.38-7.22 (m, 5H), 5.95-5.99 (m, 1H), 5.28-4.89 (m, 2H), 3.26-3.19 (m, 0.5H), 2.74-2.68 (m, 0.5 H), 2.58-2.53 (m, 0.5 H), 2.13-2.08 (m, 0.5 H).

### Step 8: 5-(2-fluoro-4-(7-hydroxy-3-oxo-5-phenyl-3,5,6,7-tetrahydro-2H-pyrrolo [2,1-c][1,2,4]triazol-2-yl)phenoxy)-4-methylthiazole-2-carboxamide

To a solution of 7-hydroxy-5-phenyl-6,7-dihydro-2H-pyrrolo[2,1-c][1,2,4]triazol-3(5H)-one (1.00 eq, 200 mg, 0.92 mmol) in DMF (4 mL), was added CuI (0.500 eq, 88 mg, 0.46 mmol), K₂CO₃ (2.00 eq, 254 mg, 1.84 mmol), 5-(4-bromo-2-fluorophenoxy)-4-methylthiazole-2-carboxamide (1.00 eq, 302 mg, 0.92 mmol), (1R,2R)—N1,N2-dimethylcyclohexane-1,2-diamine (1.00 eq, 132 mg, 0.92 mmol). The reaction mixture was stirred for 1 h at 110° C. under nitrogen. The reaction was quenched with water (20 mL) and extracted with EtOAc (20 mL×3). The combined organic layers were washed with brine (4 mL), dried over anhydrous Na₂SO₄, and after filtration, the filtrate was concentrated under reduced pressure. The residue was purified by silica gel column chromatography, eluted with (PE:EtOAc) (1:2) to afford 5-(2-fluoro-4-(7-hydroxy-3-oxo-5-phenyl-3,5,6,7-tetrahydro-2H-pyrrolo[2,1-c][1,2,4]triazol-2-yl)phenoxy)-4-methylthiazole-2-carboxa mide (300 mg, 70% yield) as a brown yellow oil. LC-MS (m/z): 468.1[M+H]⁺.

### Step 9: 5-(2-fluoro-4-(7-fluoro-3-oxo-5-phenyl-3,5,6,7-tetrahydro-2H-pyrrolo [2,1-c][1,2,4]triazol-2-yl)phenoxy)-4-methylthiazole-2-carboxamide

To a solution of 5-(2-fluoro-4-(7-hydroxy-3-oxo-5-phenyl-3,5,6,7-tetrahydro-2H-pyrrolo[2,1-c][1,2,4]triazol-2-yl)phenoxy)-4-methylthiazole-2-carboxamide (1.00 eq, 77 mg, 0.16 mmol) in DCM (1 mL), was added dropwise DAST (1.5 eq, 17 L 0.24 mmol) in DCM(0.6 mL) and stirred for 1 h at 0° C. under nitrogen. The reaction was quenched with sat. NaHCO₃ (2 mL) and extracted with DCM (10 mL×3), The combined Organic layers were dried over anhydrous Na₂SO₄, and after filtration, the filtrate was concentrated under reduced pressure. The residue was purified by silica gel column chromatography, eluted with (PE:EtOAc) (1:1) to afford 5-(2-fluoro-4-(7-fluoro-3-oxo-5-phenyl-3,5,6,7-tetrahydro-2H-pyrrolo[2,1-c][1,2,4]triazol-2-yl)phenoxy)-4-methylthiazole-2-carboxamide: polar spot (35 mg, 45% yield) and less polar spot (28.7 mg, 37% yield) as a brown yellow oil. LC-MS (m/z):470.1 [M+H]⁺.

| Compound No. | | | Appearance | | |
|---|---|---|---|---|---|
| Method | Activity | Structure and Name | and Yield | ¹H NMR Data | MS(m/z) [M + H]⁺ |
| Example 80 Method 12 Polar spot | ++++ | 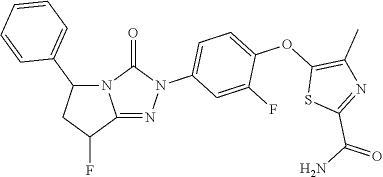 [SMILES: Cc1nc(C(N)=O)sc1Oc1ccc(-n2nc3n(c2=O)C(c2ccccc2)CC3F)cc1F] | brown yellow oil, yield: 45% | NA | 470.1 |
| | | 5-(2-fluoro-4-(7-fluoro-3-oxo-5- | | | |
| | | phenyl-3,5,6,7-tetrahydro-2H- | | | |
| | | pyrrolo[2,1-c][1,2,4]triazol-2-yl) | | | |
| | | phenoxy)-4-methylthiazole-2- | | | |
| | | carboxamide | | | |
| Example 81 Method 12 Less polar spot | ++++ | 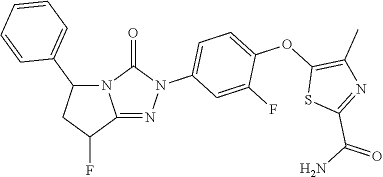 [SMILES: Cc1nc(C(N)=O)sc1Oc1ccc(-n2nc3n(c2=O)C(c2ccccc2)CC3F)cc1F] | brown yellow oil, yield: 37% | NA | 470.1 |
| | | 5-(2-fluoro-4-(7-fluoro-3-oxo-5- | | | |
| | | phenyl-3,5,6,7-tetrahydro-2H- | | | |
| | | pyrrolo[2,1-c][1,2,4]triazol-2-yl) | | | |
| | | phenoxy)-4-methylthiazole-2- | | | |
| | | carboxamide | | | |

### Example 82: Method 13

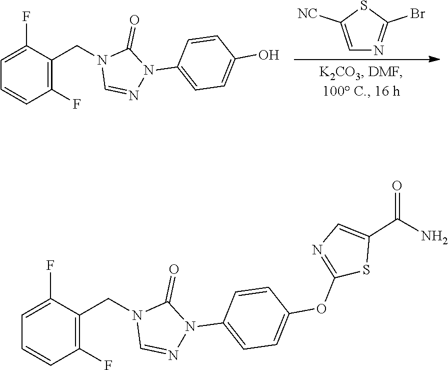 [SMILES: O=c1n(Cc2c(F)cccc2F)cnn1-c1ccc(O)cc1 ; N#Cc1cnc(Br)s1 ; NC(=O)c1cnc(Oc2ccc(-n3ncn(Cc4c(F)cccc4F)c3=O)cc2)s1]

### 2-(4-(4-(2,6-difluorobenzyl)-5-oxo-4,5-dihydro-1H-1,2,4-triazol-1-yl)phenoxy)thiazole-5-carboxamide

A solution of 4-[(2,6-difluorophenyl)methyl]-2-(4-hydroxyphenyl)-1,2,4-triazol-3-one (80 mg, 0.26 mmol), 2-bromo-1,3-thiazole-5-carbonitrile (75 mg, 0.4 mmol), K₂CO₃ (73 mg, 0.5 mmol) in DMF (2 mL) was stirred at 100° C. for 16 h. The resulted mixture was diluted with water (20 mL). The resulted mixture was extracted with EtOAc (3×10 mL). The combined Organic layers were washed with brine (20 mL), dried over anhydrous Na₂SO₄, and after filtration, the filtrate was concentrated under reduced pressure. The residue was purified by Prep-TLC (EtOAc: PE=1:1) to afford the product (25.1 mg, 20.8% yield) as a white solid. LC-MS (m/z): 430.0 [M+H]⁺. ¹H NMR (400 MHz, CDCl₃) δ 8.08 (d, J=9.1 Hz, 2H), 7.61 (d, J=15.1 Hz, 2H), 7.42-7.31 (m, 3H), 7.04-6.94 (m, 2H), 5.61 (s, 2H), 4.99 (s, 2H).

| Com- | | | Appear- | | MS |
|---|---|---|---|---|---|
| pound | | | ance | | (m/z) |
| No. | | | and | | [M + |
| Method | Activity | Structure and Name | Yield | ¹H NMR Data | H]⁺ |
| Ex- am- ple 82 Meth- od 13 | +++ | 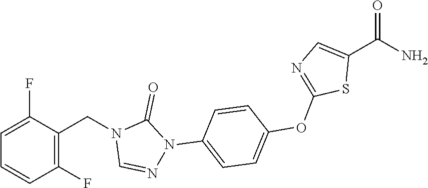 [SMILES: NC(=O)c1cnc(Oc2ccc(-n3ncn(Cc4c(F)cccc4F)c3=O)cc2)s1] | white solid, yield: 28.8% | ¹H NMR (400 MHz, CDCl₃) δ 8.08 (d, J = 9.1 Hz, 2H), 7.61 (d, J = 15.1 Hz, 2H), 7.42-7.31 (m, 3H), 7.04- 6.94 (m, 2H), 5.61 (s, 2H), 4.99 (s, 2H). | 430.0 |
| | | 2-(4-(4-(2,6-difluorobenzyl)-5- | | | |
| | | oxo-4,5-dihydro-1H-1,2,4-triazol- | | | |
| | | 1-yl)phenoxy)thiazole-5- | | | |
| | | carboxamide | | | |

### Example 83: Method 14

### 4-(2,6-difluorobenzyl)-2-(4-((4,5,6,7-tetrahydrobenzo[d]thiazol-2-yl)oxy)phenyl)-2,4-dihy dro-3H-1,2,4-triazol-3-one

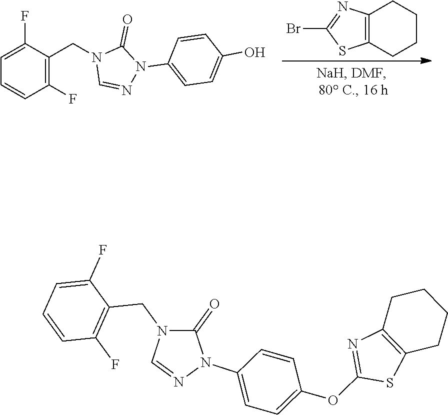 [SMILES: O=c1n(Cc2c(F)cccc2F)cnn1-c1ccc(O)cc1 ; Brc1nc2c(s1)CCCC2 ; O=c1n(Cc2c(F)cccc2F)cnn1-c1ccc(Oc2nc3c(s2)CCCC3)cc1]

A solution of 4-[(2,6-difluorophenyl)methyl]-2-(4-hydroxyphenyl)-1,2,4-triazol-3-one (50 mg, 0.16 mmol), 2-bromo-4,5,6,7-tetrahydro-1,3-benzothiazole (72 mg, 0.3 mmol), NaH (8 mg, 0.2 mmol) in DMF (3 mL) was stirred at 80° C. for 16 h. The resulted mixture was diluted with water (20 mL). The resulted mixture was extracted with EtOAc (3×10 mL). The combined organic layers were washed with brine (20 mL), dried over anhydrous Na₂SO₄, and after filtration, the filtrate was concentrated under reduced pressure. The residue was purified by Prep-HPLC to afford 4-(2,6-difluorobenzyl)-2-(4-((4,5,6,7-tetrahydrobenzo[d]thiazol-2-yl)oxy)phenyl)-2,4-dihydro-3H-1,2,4-triazol-3-one (1.1 mg, 1.5% yield) as a white solid. ¹H NMR (400 MHz, CDCl₃) δ 8.00 (d, J=9.1 Hz, 2H), 7.58 (s, 1H), 7.37 (s, 1H), 7.29 (d, J=9.1 Hz, 2H), 7.02-6.95 (m, 2H), 4.98 (s, 2H), 2.62 (d, J=5.6 Hz, 4H), 1.85-1.81 (m, 4H).

| Com- | | | Appear- | | MS |
|---|---|---|---|---|---|
| pound | | | ance | | (m/z) |
| No. | | | and | | [M + |
| Method | Activity | Structure and Name | Yield | ¹H NMR Data | H]⁺ |
| Ex- am- ple 83 Meth- od 14 | +++ | 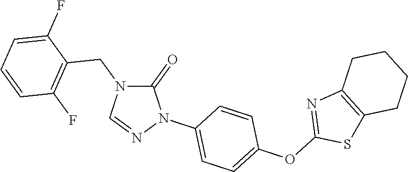 [SMILES: O=c1n(Cc2c(F)cccc2F)cnn1-c1ccc(Oc2nc3c(s2)CCCC3)cc1] | white solid, yield: 1.5% | ¹H NMR (400 MHz, CDCl₃) δ 8.00 (d, J = 9.1 Hz, 2H), 7.58 (s, 1H), 7.37 (s, 1H), 7.29 (d, J = 9.1 Hz, 2H), 7.02-6.95 (m, 2H), 4.98 (s, 2H), 2.62 (d, J = 5.6 Hz, 4H), 1.85-1.81 (m, 4H). | 441.1 |
| | | 4-(2,6-difluorobenzyl)-2-(4- | | | |
| | | ((4,5,6,7-tetrahydrobenzo[d]thiazol- | | | |
| | | 2-yl)oxy)phenyl)-2,4-dihydro- | | | |
| | | 3H-1,2,4-triazol-3-one | | | |

### Example 84: Method 15

### 4-(2,6-difluorobenzyl)-2-(4-((2-(3-ethoxyazetidin-1-yl)thiazol-5-yl)oxy)phenyl)-2,4-dihydro-3H-1,2,4-triazol-3-one

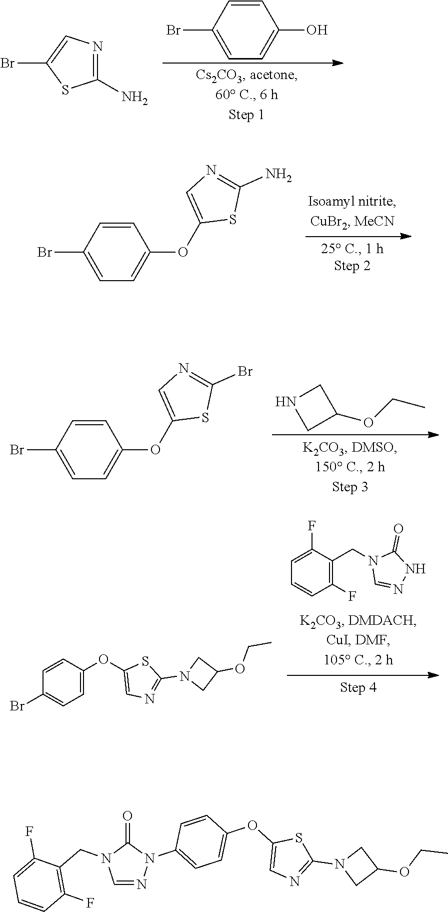

### Step 1: 5-(4-bromophenoxy)thiazol-2-amine

To a stirred solution of 5-bromothiazol-2-amine (5 g, 27.9 mmol) in acetone (100 mL) were added 4-bromophenol (4.8 g, 27.9 mmol) and Cs₂CO₃ (18.2 g, 56 mmol). The resulted mixture was stirred for additional 6 h at 60° C. under N₂. The mixture was allowed to cool down to rt. The resulted mixture was diluted with water (100 mL). The resulted mixture was extracted with EtOAc (3×100 mL). The combined Organic layers were washed with brine (100 mL), dried over anhydrous Na₂SO₄, after filtration, the filtrate was concentrated under reduced pressure. The residue was purified by silica gel column chromatography, eluted with (PE: EtOAc=2:1) to afford 5-(4-bromophenoxy)thiazol-2-amine (460 mg, 6% yield) as a white solid. MS (m/z): 270.9 [M+H]⁺.

### Step 2: 2-bromo-5-(4-bromophenoxy)thiazole

To a stirred solution of 5-(4-bromophenoxy)-1,3-thiazol-2-amine (460 mg, 1.7 mmol) in MeCN (10 mL) was added CuBr₂ (758 mg, 3.4 mmol), and the reaction mixture was cooled to 0° C. Then Isoamyl nitrite (400 mg, 3.4 mmol) was added dropwise. The resulted mixture was stirred for additional 1 h at 25° C. The mixture was quenched with NH₄Cl (10 mL). The resulted mixture was extracted with EtOAc (3×10 mL). The combined Organic layers were washed with brine (10 mL), dried over anhydrous Na₂SO₄, and after filtration, the filtrate was concentrated under reduced pressure. The residue was purified by silica gel column chromatography, eluted with (PE: EtOAc=5:1) to afford 2-bromo-5-(4-bromophenoxy)thiazole (280 mg, 27% yield) as a white solid. MS (m/z): 335.8 [M+H]⁺.

### Step 3: 5-(4-bromophenoxy)-2-(3-ethoxyazetidin-1-yl)thiazole

To a stirred solution of 2-bromo-5-(4-bromophenoxy)thiazole (280 mg, 0.83 mmol) and 3-ethoxyazetidine (115 mg, 0.83 mmol) in DMSO (5 mL) were added K₂CO₃ (345 mg, 2.5 mmol). The resulted mixture was stirred for additional 2 h at 150° C. under N₂. The mixture was allowed to cool down to rt. The resulted mixture was diluted with water (10 mL). The resulted mixture was extracted with EtOAc (3×10 mL). The combined Organic layers were washed with brine (10 mL), dried over anhydrous Na₂SO₄, after filtration, the filtrate was concentrated under reduced pressure. The residue was purified by silica gel column chromatography, eluted with (PE:EtOAc=1:1) to afford 5-(4-bromophenoxy)-2-(3-ethoxyazetidin-1-yl)thiazole (140 mg, 47% yield) as a white solid. MS (m/z): 354.9 [M+H]⁺.

### Step 4: 4-(2,6-difluorobenzyl)-2-(4-((2-(3-ethoxyazetidin-1-yl)thiazol-5-yl)oxy)phenyl)-2,4-dihydro-3H-1,2,4-triazol-3-one

To a stirred solution of 5-(4-bromophenoxy)-2-(3-ethoxyazetidin-1-yl)thiazole (130 mg, 0.366 mmol) and 4-(2,6-difluorobenzyl)-2,4-dihydro-3H-1,2,4-triazol-3-one (78 mg, 0.366 mmol) in DMF (5 mL) were added (1S,2S)—N¹,N²-dimethylcyclohexane-1,2-diamine (25 mg, 0.18 mmol), CuI (34 mg, 0.18 mmol), K₂CO₃ (138 mg, 1 mmol). The resulted mixture was stirred for additional 2 h at 105° C. under N₂. The mixture was allowed to cool down to rt. The resulted mixture was diluted with water (10 mL). The resulted mixture was extracted with EtOAc (3×10 mL). The combined Organic layers were washed with brine (10 mL), dried over anhydrous Na₂SO₄, and after filtration, the filtrate was concentrated under reduced pressure. The residue was purified by reverse phase flash chromatography with the following conditions: column: Spherical C18 40-60 um, 40 g; Mobile phase B: ACN; Flow rate: 40 mL/min; Gradient: 35% B-60% B in 20 min; Detector: 254 nm. The fractions containing the desired product were concentrated under reduced pressure to afford 4-(2,6-difluorobenzyl)-2-(4-((2-(3-ethoxyazetidin-1-yl)thiazol-5-yl)oxy)phenyl)-2,4-dihydro-3H-1,2,4-triazol-3-one (24 mg, 13% yield) as an off-white solid. LC-MS (m/z): 486.1 [M+H]⁺. ¹H NMR (400 MHz, CDCl₃) δ 7.94-7.84 (m, 2H), 7.56 (s, 1H), 7.35 (td, J=8.4, 4.2 Hz, 1H), 7.14-7.05 (m, 2H), 6.98 (p, J=3.5 Hz, 2H), 6.85 (s, 1H), 4.97 (s, 2H), 4.52-4.39 (m, 1H), 4.30-4.16 (m, 2H), 3.96 (dd, J=8.8, 4.4 Hz, 2H), 3.48 (q, J=7.0 Hz, 2H), 1.24 (t, J=7.0 Hz, 3H).

Example (Compound) 85 was synthesized using a similar method to that used in Example 84.

| Com- | | | Ap- | | |
|---|---|---|---|---|---|
| pound | | | pear- | | MS |
| No. | | | ance | | (m/z) |
| Meth- | Ac- | | and | | [M + |
| od | tivity | Structure and Name | Yield | ¹H NMR Data | H]⁺ |
| Ex- am- ple 84 Meth- od 15 | ++++ | 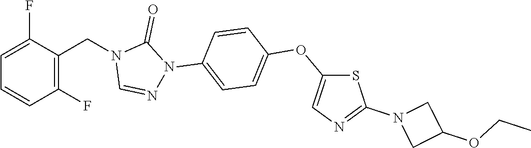 [SMILES: CCOC1CN(c2ncc(Oc3ccc(-n4ncn(Cc5c(F)cccc5F)c4=O)cc3)s2)C1] | off- white solid, yield: 13% | ¹H NMR (400 MHz, CDCl₃) δ 7.94-7.84 (m, 2H), 7.56 (s, 1H), 7.35 (td, J = 8.4, 4.2 Hz, 1H), 7.14-7.05 (m, 2H), 6.98 (p, J = 3.5 Hz, 2H), 6.85 (s, 1H), 4.97 (s, 2H), 4.52-4.39 (m, 1H), 4.30- 4.16 (m, 2H), 3.96 (dd, J = 8.8, 4.4 Hz, 2H), 3.48 (q, J = 7.0 Hz, 2H), 1.24 (t, J = 7.0 Hz, 3H). | 486.1 |
| | | 4-(2,6-difluorobenzyl)-2-(4-((2- | | | |
| | | (3-ethoxyazetidin-1-yl)thiazol-5- | | | |
| | | yl)oxy)phenyl)-2,4-dihydro-3H- | | | |
| | | 1,2,4-triazol-3-one | | | |
| Ex- am- ple 85 Meth- od 15 | + | 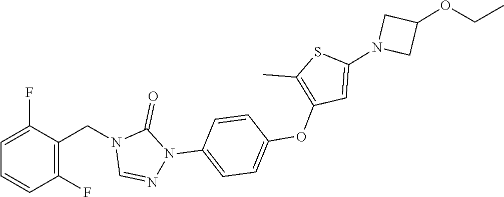 [SMILES: CCOC1CN(c2cc(Oc3ccc(-n4ncn(Cc5c(F)cccc5F)c4=O)cc3)c(C)s2)C1] | white solid, yield: 6.6% | ¹H NMR (400 MHz, DMSO-d₆) δ 9.49 (s, 1H), 7.64-7.57 (m, 2H), 7.37-7.26 (m, 1H), 6.97 (t, J = 8.2 Hz, 2H), 6.83-6.77 (m, 2H), 5.81 (s, 1H), 4.91 (s, 2H), 4.37- 4.28 (m, 1H), 4.01-3.91 (m, 2H), 3.52 (dd, J = 8.2, 4.8 Hz, 2H), 3.39 (q, J = 7.0 Hz, 2H), 2.11 (s, 3H), 1.10 (t, J = 7.0 Hz, 3H). | 499.2 |
| | | 4-(2,6-difluorobenzyl)-2-(4-((5- | | | |
| | | (3-ethoxyazetidin-1-yl)-2-methyl- | | | |
| | | thiophen-3-yl)oxy)phenyl)-2,4- | | | |
| | | dihydro-3H-1,2,4-triazol-3-one | | | |

### Example 86: Method 16

### 5-(2-chloro-4-(4-(2,6-difluorobenzyl)-5-oxo-4,5-dihydro-1H-1,2,4-triazol-1-yl)phenoxy)-4-methylthiazole-2-carbonitrile

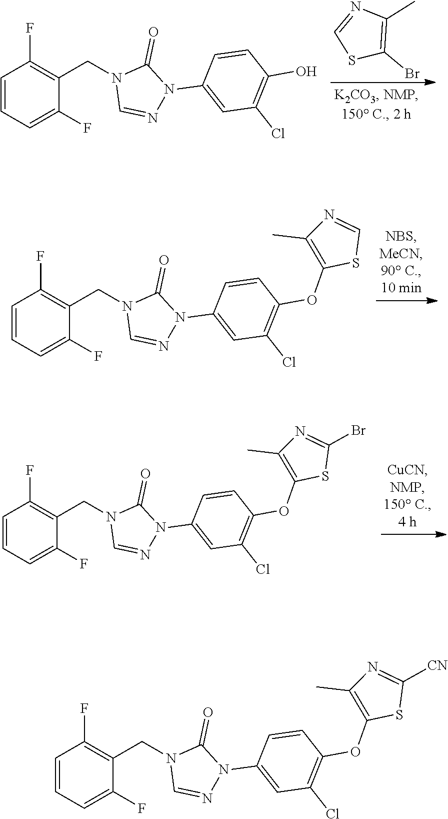

### Step 1: 2-(3-chloro-4-((4-methylthiazol-5-yl)oxy)phenyl)-4-(2,6-difluorobenzyl)-2,4-dihydro-3H-1,2,4-triazol-3-one

To a stirred solution of 2-(3-chloro-4-hydroxyphenyl)-4-(2,6-difluorobenzyl)-2,4-dihydro-3H-1,2,4-triazol-3-one (2 g, 5.92 mmol) and 5-bromo-4-methylthiazole (1.27 g, 7.11 mmol) in NMP (100 mL) were added K₂CO₃ (2.46 g, 17.77 mmol) at rt. The resulted mixture was stirred for additional 2 h at 150° C. The mixture was allowed to cool down to rt. The resulted mixture was diluted with water (200 mL). The resulted mixture was extracted with EtOAc (3×150 mL). The combined Organic layers were washed with brine (100 mL), dried over anhydrous Na₂SO₄, and after filtration, the filtrate was concentrated under reduced pressure. The residue was purified by silica gel column chromatography, eluted with (PE: EtOAc=1:0 to 1:1) to afford 2-(3-chloro-4-((4-methylthiazol-5-yl)oxy)phenyl)-4-(2,6-difluorobenzyl)-2,4-dihydro-3H-1,2,4-triazol-3-one (2.6 g, quantitative) as a brown yellow solid. MS (m/z): 436.2 [M+H]⁺.

### Step 2: 2-(4-((2-bromo-4-methylthiazol-5-yl)oxy)-3-chlorophenyl)-4-(2,6-difluorobenzyl)-2,4-dihydro-3H-1,2,4-triazol-3-one

To a stirred solution of 2-(3-chloro-4-((4-methylthiazol-5-yl)oxy)phenyl)-4-(2,6-difluorobenzyl)-2,4-dihydro-3H-1,2,4-triazol-3-one (2.6 g, 5.98 mmol) in MeCN (100 mL) were added NBS (2.13 g, 11.96 mmol) at rt. The resulted mixture was stirred for additional 10 min at 90° C. The mixture was allowed to cool down to rt. The resulted mixture was diluted with water (40 mL). The resulted mixture was extracted with EtOAc (3×80 mL). The combined Organic layers were washed with brine (80 mL), dried over anhydrous Na₂SO₄, and after filtration, the filtrate was concentrated under reduced pressure. The residue was purified by silica gel column chromatography, eluted with (PE: EtOAc=1:0-1:1) to afford 2-(4-((2-bromo-4-methylthiazol-5-yl)oxy)-3-chlorophenyl)-4-(2,6-difluorobenzyl)-2,4-dihydro-3H-1,2,4-triazol-3-one (1.68 g, 54.7% yield) as a brown solid. MS (m/z): 513.1 [M+H]⁺.

### Step 3: 5-(2-chloro-4-(4-(2,6-difluorobenzyl)-5-oxo-4,5-dihydro-1H-1,2,4-triazol-1-yl) phenoxy)-4-methylthiazole-2-carbonitrile

To a stirred solution of 2-(4-((2-bromo-4-methylthiazol-5-yl)oxy)-3-chlorophenyl)-4-(2,6-difluorobenzyl)-2,4-dihydro-3H-1,2,4-triazol-3-one (300 mg, 0.58 mmol) in NMP (15 mL) was added CuCN (131 mg, 1.46 mmol) at rt. The resulted mixture was stirred for additional 4 h at 150° C. The mixture was allowed to cool down to rt. The resulted mixture was diluted with water (40 mL). The resulted mixture was extracted with EtOAc (3×80 mL). The combined Organic layers were washed with brine (80 mL), dried over anhydrous Na₂SO₄, and after filtration, the filtrate was concentrated under reduced pressure. The residue was purified by silica gel column chromatography, eluted with (PE: EtOAc=1:0 to 1:1) to afford 5-(2-chloro-4-(4-(2,6-difluorobenzyl)-5-oxo-4,5-dihydro-1H-1,2,4-triazol-1-yl)phenoxy)-4-met hylthiazole-2-carbonitrile (1.2 mg, 0.45% yield) as a white solid. LC-MS (m/z): 461.1[M+H]⁺. ¹H NMR (400 MHz, CDCl₃) δ 8.22 (d, J=2.5 Hz, 1H), 7.98-7.93 (m, 1H), 7.61 (s, 1H), 7.37 (td, J=8.3, 4.1 Hz, 1H), 7.11 (d, J=9.0 Hz, 1H), 6.99 (t, J=7.9 Hz, 2H), 4.98 (s, 2H), 2.43 (s, 3H).

Examples (Compounds) 87-88 were synthesized using a similar method to that used in Example 86.

| Com- | | | Ap- | | |
|---|---|---|---|---|---|
| pound | | | pear- | | |
| No. | | | ance | | |
| Meth- | | | and | | MS(m/z) |
| od | Activity | Structure and Name | Yield | ¹H NMR Data | [M + H]⁺ |
| Ex- am- ple 86 Meth- od 16 | ++++ | 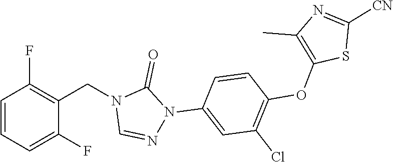 [SMILES: Cc1nc(C#N)sc1Oc1ccc(-n2ncn(Cc3c(F)cccc3F)c2=O)cc1Cl] | white solid, yield: 0.45% | ¹H NMR (400 MHz, CDCl₃) δ 8.22 (d, J = 2.5 Hz, 1H), 7.98- 7.93 (m, 1H), 7.61 (s, 1H), 7.37 (td, J = 8.3, 4.1 Hz, 1H), 7.11 (d, J = 9.0 Hz, 1H), 6.99 (t, J = 7.9 Hz, 2H), 4.98 (s, 2H), 2.43 (s, 3H). | 460.1 |
| | | 5-(2-chloro-4-(4-(2,6-difluoro- | | | |
| | | benzyl)-5-oxo-4,5-dihydro-1H- | | | |
| | | 1,2,4-triazol-1-yl)phenoxy)-4- | | | |
| | | methylthiazole-2-carbonitrile | | | |
| Ex- am- ple 87 Meth- od 16 | ++++ | 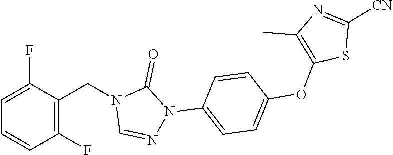 [SMILES: Cc1nc(C#N)sc1Oc1ccc(-n2ncn(Cc3c(F)cccc3F)c2=O)cc1] | white solid, yield: 60.8% | ¹H NMR (400 MHz, DMSO-d₆) δ 8.33 (s, 1H), 7.91 (d, J = 9.1 Hz, 2H), 7.56-7.41 (m, 1H), 7.34 (d, J = 9.1 Hz, 2H), 7.16 (t, J = 8.1 Hz, 2H), 4.98 (s, 2H), 2.32 (s, 3H). | 426.1 |
| | | 5-(4-(4-(2,6-difluorobenzyl)-5- | | | |
| | | oxo-4,5-dihydro-1H-1,2,4- | | | |
| | | triazol-1-yl)phenoxy)-4- | | | |
| | | methylthiazole-2-carbonitrile | | | |
| Ex- am- ple 88 Meth- od 16 | ++++ | 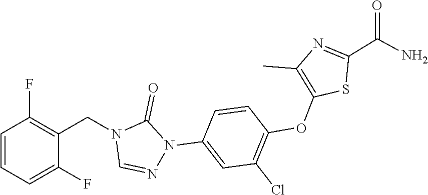 [SMILES: Cc1nc(C(N)=O)sc1Oc1ccc(-n2ncn(Cc3c(F)cccc3F)c2=O)cc1Cl] | white solid, yield: 0.45% | ¹H NMR (400 MHz, CDCl₃) δ 8.18 (d, J = 2.5 Hz, 1H), 7.89 (dd, J = 9.0, 2.5 Hz, 1H), 7.59 (s, 1H), 7.41-7.32 (m, 1H), 7.03 (d, J = 9.0 Hz, 1H), 6.99 (t, J = 7.9 Hz, 3H), 4.97 (s, 2H), 2.34 (s, 3H). | 478.1 |
| | | 5-(2-chloro-4-(4-(2,6-difluoro- | | | |
| | | benzyl)-5-oxo-4,5-dihydro-1H- | | | |
| | | 1,2,4-triazol-1-yl)phenoxy)-4- | | | |
| | | methylthiazole-2-carboxamide | | | |

### Example 89: Method 17

### N-(5-(4-(4-(2,6-difluorobenzyl)-5-oxo-4,5-dihydro-1H-1,2,4-triazol-1-yl)-2-fluorophenoxy)-4-methylthiazol-2-yl)acetamide

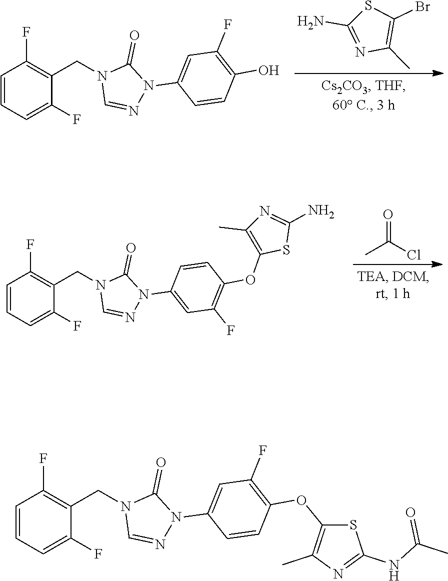

### Step 1: 2-(4-((2-amino-4-methylthiazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(3-fluoro-4-hydroxyphenyl)-2,4-dihydro-3H-1,2,4-triazol-3-one (400 mg, 1.25 mmol) and 5-bromo-4-methylthiazol-2-amine (264 mg, 1.37 mmol) in DMF (20 mL) were added Cs₂CO₃ (1.22 g, 3.74 mmol) at rt. The resulted mixture was stirred for additional 3 h at 60° C. The mixture was allowed to cool down to rt. The resulted mixture was diluted with water (100 mL). The resulted mixture was extracted with EtOAc (3×80 mL). The combined organic layers were washed with brine (100 mL), dried over anhydrous Na₂SO₄, and after filtration, the filtrate was concentrated under reduced pressure. The residue was purified by silica gel column chromatography, eluted with (PE: EtOAc=1:0 to 1:1) to afford 2-(4-((2-amino-4-methylthiazol-5-yl)oxy)-3-fluorophenyl)-4-(2,6-difluorobenzyl)-2,4-dihydro-3H-1,2,4-triazol-3-one (140 mg, 25.94% yield) as a brown yellow solid. MS (m/z): 434.3 [M+H]⁺.

### Step 2: N-(5-(4-(4-(2,6-difluorobenzyl)-5-oxo-4,5-dihydro-1H-1,2,4-triazol-1-yl)-2-fluorophenoxy)-4-methylthiazol-2-yl)acetamide

To a stirred solution of 2-(4-((2-amino-4-methylthiazol-5-yl)oxy)-3-fluorophenyl)-4-(2,6-difluorobenzyl)-2,4-dihydro-3H-1,2,4-triazol-3-one (140 mg, 0.32 mmol) and TEA (98 mg, 0.97 mmol) in DCM (15 mL) were added acetyl chloride (28 mg, 0.36 mmol) at rt. The resulted mixture was stirred for additional 2 h at rt. The mixture was concentrated under reduced pressure. The residue was purified by prep-HPLC to afford N-(5-(4-(4-(2,6-difluorobenzyl)-5-oxo-4,5-dihydro-1H-1,2,4-triazol-1-yl)-2-fluorophenoxy)-4-methylthiazol-2-yl)acetamide (10.4 mg, 6.77% yield) as a white solid. MS (m/z): 476.1 [M+H]⁺. ¹H NMR (400 MHz, CDCl₃) δ 7.88 (dd, J=12.3, 2.2 Hz, 1H), 7.70 (d, J=9.1 Hz, 1H), 7.57 (s, 1H), 7.42-7.31 (m, 1H), 7.03-6.94 (m, 3H), 4.97 (s, 2H), 2.23 (s, 3H), 2.19 (s, 3H).

Examples (Compounds) 90-91 were synthesized using a similar method to that used in Example 89.

| Com- | | | Appear- | | MS |
|---|---|---|---|---|---|
| pound | | | ance | | (m/z) |
| No. | | | and | | [M + |
| Method | Activity | Structure and Name | Yield | ¹H NMR Data | H]⁺ |
| Ex- am- ple 89 Method 17 | ++++ | 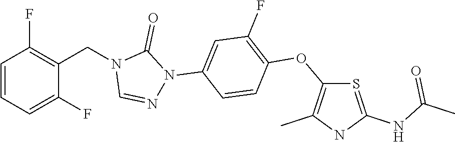 | white solid, yield: 6.77% | ¹H NMR (400 MHz, CDCl₃) δ 7.88 (dd, J = 12.3, 2.2 Hz, 1H), 7.70 (d, J = 9.1 Hz, 1H), 7.57 (s, 1H), 7.42-7.31 (m, 1H), 7.03- 6.94 (m, 3H), 4.97 (s, 2H), 2.23 (s, 3H), 2.19 (s, 3H). | 476.1 |
| | | N-(5-(4-(4-(2,6-difluorobenzyl)- | | | |
| | | 5-oxo-4,5-dihydro-1H-1,2,4- | | | |
| | | triazol-1-yl)-2-fluorophenoxy)-4- | | | |
| | | methylthiazol-2-yl)acetamide | | | |
| Ex- am- ple 90 Method 17 | +++ | 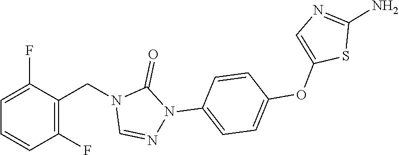 [SMILES: Nc1ncc(Oc2ccc(-n3ncn(Cc4c(F)cccc4F)c3=O)cc2)s1] | white solid, yield: | ¹H NMR (400 MHz, CDCl₃) δ ppm 7.93-7.87 (m, 2H), 7.57 (s, 1H), 7.36 (m, 1H), 7.15-7.08 (m, 2H), 7.03-6.93 (m, 2H), 6.77 (s, 1H), 4.97 (s, 2H). | 402.1 |
| | | 2-(4-((2-aminothiazol-5-yl)oxy) | | | |
| | | phenyl)-4-(2,6-difluorobenzyl)- | | | |
| | | 2,4-dihydro-3H-1,2,4-triazol-3- | | | |
| | | one | | | |
| Ex- am- ple 91 Meth- od 17 | ++++ | 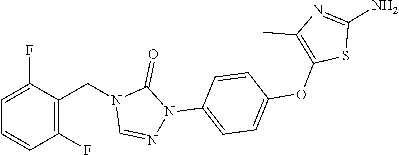 [SMILES: Cc1nc(N)sc1Oc1ccc(-n2ncn(Cc3c(F)cccc3F)c2=O)cc1] | white solid, yield: | ¹H NMR (400 MHz, CDCl₃) δ ppm 7.91-7.82 (m, 2H), 7.57 (s, 1H), 7.42-7.32 (m, 1H), 7.09- 7.02 (m, 2H), 7.01-6.93 (m, 2H), 4.97 (s, 2H), 2.06 (s, 3H). | 416.1 |
| | | 2-(4-((2-amino-4-methylthiazol- | | | |
| | | 5-yl)oxy)phenyl)-4-(2,6-difluoro- | | | |
| | | benzyl)-2,4-dihydro-3H-1,2,4- | | | |
| | | triazol-3-one | | | |

### Example 92: Method 18

### methyl 2-(4-(4-(2,6-difluorobenzyl)-5-oxo-4,5-dihydro-1H-1,2,4-triazol-1-yl)phenoxy)-5-methylthiazole-4-carboxylate

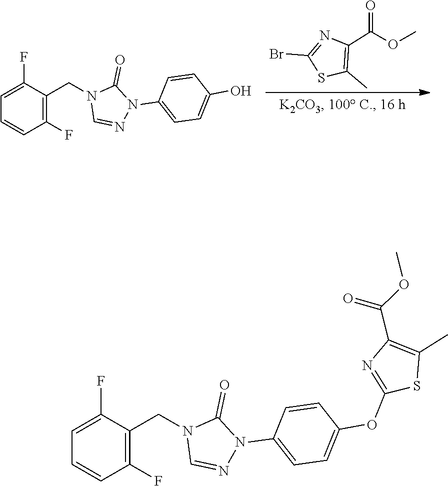 [SMILES: O=c1n(Cc2c(F)cccc2F)cnn1-c1ccc(O)cc1 ; COC(=O)c1nc(Br)sc1C ; COC(=O)c1nc(Oc2ccc(-n3ncn(Cc4c(F)cccc4F)c3=O)cc2)sc1C]

A solution of 4-[(2,6-difluorophenyl)methyl]-2-(4-hydroxyphenyl)-1,2,4-triazol-3-one (80 mg, 0.26 mmol), methyl 2-bromo-5-methyl-1,3-thiazole-4-carboxylate (93 mg, 0.4 mmol), K₂CO₃ (73 mg, 0.52 mmol) in DMF (2 mL) was stirred at 100° C. for 16 h. The resulted mixture was diluted with water (20 mL). The resulted mixture was extracted with EtOAc (3×10 mL). The combined organic layers were washed with brine (20 mL), dried over anhydrous Na₂SO₄, and after filtration, the filtrate was concentrated under reduced pressure. The residue was purified by Prep-TLC (EtOAc:PE=1:1) to afford the product (20.1 mg, 15.8% yield) as a white solid. MS (m/z): 459.0 [M+H]⁺. ¹H NMR (400 MHz, CDCl₃) δ 8.09-7.98 (m, 2H), 7.59 (s, 1H), 7.40-7.28 (m, 3H), 7.04-6.94 (m, 2H), 4.98 (s, 2H), 3.88 (s, 3H), 2.67 (s, 3H).

Examples (Compounds) 93-96 were synthesized using a similar method to that used in Example 92.

| | | | Appear- | | |
|---|---|---|---|---|---|
| Compound | | | ance | | |
| No. | | | and | | MS(m/z) |
| Method | Activity | Structure and Name | Yield | ¹H NMR Data | [M + H]⁺ |
| Example 92 Method 18 | +++ | 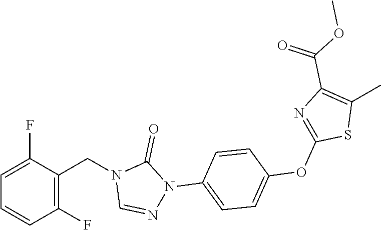 [SMILES: COC(=O)c1nc(Oc2ccc(-n3ncn(Cc4c(F)cccc4F)c3=O)cc2)sc1C] methyl 2-(4-(4-(2,6-difluorobenzyl)-5-oxo-4,5-dihydro-1H-1,2,4- triazol-1-yl)phenoxy)-5-methylthiazole-4-carboxylate | white solid, yield: 15.8% | ¹H NMR (400 MHZ, CDCl₃) δ 8.09-7.98 (m, 2H), 7.59 (s, 1H), 7.40-7.28 (m, 3H), 7.04-6.94 (m, 2H), 4.98 (s, 2H), 3.88 (s, 3H), 2.67 (s, 3H). | 459.0 |
| Example 93 Method 18 | +++ | 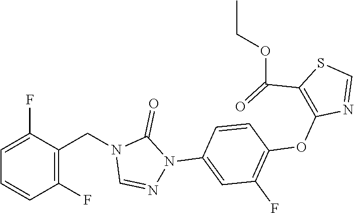 [SMILES: CCOC(=O)c1scnc1Oc1ccc(-n2ncn(Cc3c(F)cccc3F)c2=O)cc1F] ethyl 4-(4-(4-(2,6-difluorobenzyl)-5-oxo-4,5-dihydro-1H-1,2,4- triazol-1-yl)-2-fluorophenoxy)thiazole-5-carboxylate | yellow solid, yield: 60.7% | ¹H NMR (400 MHZ, CDCl₃) δ 8.54 (s, 1H), 7.93 (dd, J = 12.0, 2.3 Hz, 1H), 7.83 (d, J = 9.0 Hz, 1H), 7.57 (s, 1H), 7.37 (t, J = 7.4 Hz, 1H), 7.30 (d, J = 8.7 Hz, 1H), 6.98 (t, J = 7.8 Hz, 2H), 4.97 (s, 2H), 4.37 (d, J = 7.1 Hz, 2H), 1.37 (t, J = 7.1 Hz, 3H). | 477.0 |
| Example 94 Method 18 | ++ | 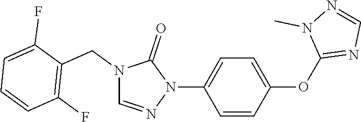 [SMILES: Cn1ncnc1Oc1ccc(-n2ncn(Cc3c(F)cccc3F)c2=O)cc1] 4-(2,6-difluorobenzyl)-2-(4-((1-methyl-1H-1,2,4-triazol-5-yl)oxy) phenyl)-2,4-dihydro-3H-1,2,4-triazol-3-one | white solid, yield: 12% | ¹H NMR (400 MHZ, CDCl₃) δ 8.08-7.99 (m, 2H), 7.58 (d, J = 3.6 Hz, 2H), 7.42-7.30 (m, 3H), 7.04-6.94 (m, 2H), 4.98 (s, 2H), 3.79 (s, 3H). | 385.1 |
| Example 95 Method 18 | +++ | 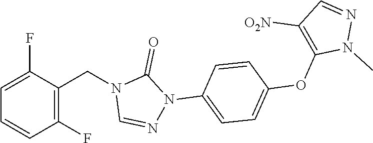 [SMILES: Cn1ncc([N+](=O)[O-])c1Oc1ccc(-n2ncn(Cc3c(F)cccc3F)c2=O)cc1] 4-(2,6-difluorobenzyl)-2-(4-((1-methyl-4-nitro-1H-pyrazol-5-yl) oxy)phenyl)-2,4-dihydro-3H-1,2,4-triazol-3-one | white solid, yield: | ¹H NMR (400 MHZ, CDCl₃) δ 8.09 (s, 1H), 7.99-7.94 (m, 2H), 7.56 (s, 1H), 7.40-7.31 (m, 1H), 7.01-6.93 (m, 4H), 4.96 (s, 2H), 3.76 (s, 3H). | 428.8 |
| Example 96 Method 18 | +++ | 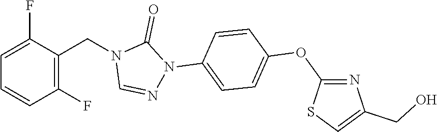 [SMILES: O=c1n(Cc2c(F)cccc2F)cnn1-c1ccc(Oc2nc(CO)cs2)cc1] 4-(2,6-difluorobenzyl)-2-(4-((4-(hydroxymethyl)thiazol-2-yl)oxy) phenyl)-2,4-dihydro-3H-1,2,4-triazol-3-one | off- white solid, yield: 46.24% | ¹H NMR (400 MHz, CDCl₃) δ 8.08-8.00 (m, 2H), 7.59 (s, 1H), 7.43-7.29 (m, 3H), 7.03-6.92 (m, 2H), 6.69 (s, 1H), 4.98 (s, 2H), 4.60 (s, 2H). | 417.4 |

### Example 97: Method 19

### 4-(2,6-difluorobenzyl)-2-(4-((2,4-dimethylthiazol-5-yl)oxy)phenyl)-2,4-dihydro-3H-1,2,4-tr iazol-3-one

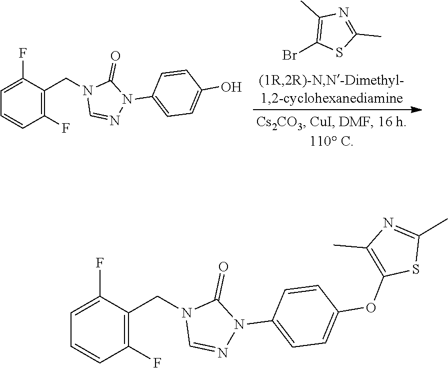

To a stirred solution of 4-[(2,6-difluorophenyl)methyl]-2-(4-hydroxyphenyl)-1,2,4-triazol-3-one (200 mg, 0.6595 mmol), 5-bromo-2,4-dimethyl-1,3-thiazole (152.01 mg, 0.7914 mmol), and 1-N,2-N-dimethylcyclohexane-1,2-diamine (18.76 mg, 0.1319 mmol) in DMF were added CuI (25.12 mg, 0.1319 mmol) and Cs₂CO₃ (537.2 mg, 1.6487 mmol). The resulted mixture was stirred for additional 16 h at 110° C. After filtration, the filtrate was concentrated under reduced pressure. The resulted mixture was diluted with water (100 mL). The resulted mixture was extracted with EtOAc (3×50 mL). The combined Organic layers were washed with brine (100 mL), dried over anhydrous Na₂SO₄, and after filtration, the filtrate was concentrated under reduced pressure. The resulted mixture was concentrated under reduced pressure. The residue was purified by Flash (PE:EA=1:2) to obtain the titled compound (65 mg, 24.48% yield) as a white solid. LC-MS (m/z): 415.4 [M+H]⁺. ¹H NMR (400 MHz, CDCl₃) δ 8.03-7.84 (m, 2H), 7.56 (s, 1H), 7.42-7.31 (m, 1H), 7.05-6.94 (m, 4H), 4.97 (s, 2H), 2.63 (s, 3H), 2.22 (s, 3H).

Examples (Compounds) 98-99 were synthesized using a similar method to that used in Example 97.

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 97 Method 19 | ++++ | 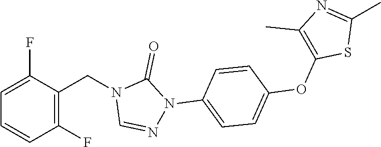 [SMILES: Cc1nc(C)c(Oc2ccc(-n3ncn(Cc4c(F)cccc4F)c3=O)cc2)s1] 4-(2,6-difluorobenzyl)-2-(4-((2,4-dimethylthiazol-5-yl)oxy)phenyl)- 2,4-dihydro-3H-1,2,4-triazol-3-one | white solid, yield: 24.48% | ¹H NMR (400 MHZ, CDCl₃) δ 8.03-7.84 (m, 2H), 7.56 (s, 1H), 7.42-7.31 (m, 1H), 7.05-6.94 (m, 4H), 4.97 (s, 2H), 2.63 (s, 3H), 2.22 (s, 3H). | 415.4 |
| Example 98 Method 19 | +++ | 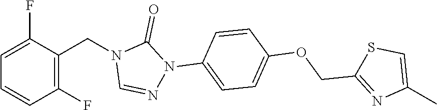 [SMILES: Cc1csc(COc2ccc(-n3ncn(Cc4c(F)cccc4F)c3=O)cc2)n1] 4-(2,6-difluorobenzyl)-2-(4-((4-methylthiazol-2-yl)methoxy) phenyl)-2,4-dihydro-3H-1,2,4-triazol-3-one | white solid, yield: | ¹H NMR (400 MHZ, CDCl₃) δ 8.01 (s, 1H), 7.85 (d, J = 9.1 Hz, 2H), 7.55 (s, 1H), 7.07-6.95 (m, 4H), 6.90 (s, 1H), 5.34 (s, 2H), 4.97 (s, 2H), 2.47 (s, 3H). | 415.4 |
| Example 99 Method 19 | ++++ | 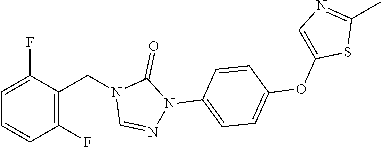 [SMILES: Cc1ncc(Oc2ccc(-n3ncn(Cc4c(F)cccc4F)c3=O)cc2)s1] 4-(2,6-difluorobenzyl)-2-(4-((2-methylthiazol-5-yl)oxy)phenyl)- 2,4-dihydro-3H-1,2,4-triazol-3-one | white solid, yield: 10.95% | ¹H NMR (400 MHZ, CDCl₃) δ 7.87-7.83 (m, 1H), 7.50 (s, 2H), 7.31-7.26 (m, 1H), 7.14 (s, 1H), 7.07-7.00 (m, 2H), 6.94-6.87 (m, 2H), 4.90 (s, 2H), 2.57 (s, 3H). | 401.4 |

### Example 100: Method 20

### 5-(4-(4-(2,6-difluorobenzyl)-5-oxo-4,5-dihydro-1H-1,2,4-triazol-1-yl)-2-fluorophenoxy)-2-methylthiazole-4-carboxamide

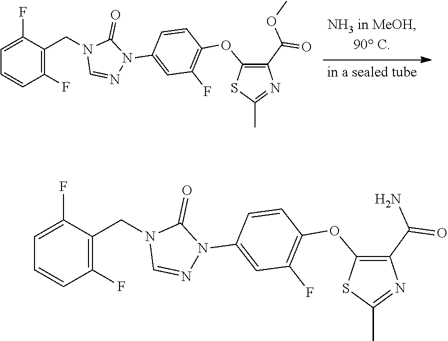 [SMILES: COC(=O)c1nc(C)sc1Oc1ccc(-n2ncn(Cc3c(F)cccc3F)c2=O)cc1F ; Cc1nc(C(N)=O)c(Oc2ccc(-n3ncn(Cc4c(F)cccc4F)c3=O)cc2F)s1]

A solution of methyl 5-(4-(4-(2,6-difluorobenzyl)-5-oxo-4,5-dihydro-1H-1,2,4-triazol-1-yl)-2-fluorophenoxy)-2-methylthiazole-4-carboxylate (332 mg, 0.697 mmol) in NH₃ in MeOH (20 mL) was stirred for 16 h at 90° C. in a sealed tube. The resulted mixture was diluted with water (20 mL). The resulted mixture was extracted with EtOAc (3×20 mL). The combined organic layers were washed with brine, dried over anhydrous Na₂SO₄, and after filtration, the filtrate was concentrated under reduced pressure. The residue was purified by reverse phase flash chromatography with the following conditions: column: Spherical C₁₈ 40-60 um, 40 g; Mobile phase B: ACN; Flow rate: 40 mL/min; Gradient: 35% B-60% B in 20 min; Detector: 254 nm. The fractions containing the desired product were collected at 51% B and concentrated under reduced pressure to afford the titled compound 5-(4-(4-(2,6-difluorobenzyl)-5-oxo-4,5-dihydro-1H-1,2,4-triazol-1-yl)-2-fluorophenoxy)-2-met hylthiazole-4-carboxamide (139 mg, 41% yield) as a white solid. LC-MS (m/z): 462.1 [M+H]⁺. ¹H NMR (400 MHz, CD₃OD) δ 8.02 (s, 1H), 7.91 (dd, J=12.3, 2.4 Hz, 1H), 7.78 (d, J=8.9 Hz, 1H), 7.50-7.41 (m, 1H), 7.35 (t, J=8.9 Hz, 1H), 7.06 (t, J=8.1 Hz, 2H), 5.04 (s, 2H), 2.58 (s, 3H).

Examples (Compounds) 101-104 were synthesized using a similar method to that used in Example 100.

| Compound | | | Appearance | | MS(m/z) |
|---|---|---|---|---|---|
| No. Method | Activity | Structure and Name | and Yield | ¹H NMR Data | [M + H]⁺ |
| Example 100 Method 20 | +++ | 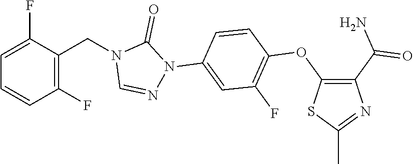 [SMILES: Cc1nc(C(N)=O)c(Oc2ccc(-n3ncn(Cc4c(F)cccc4F)c3=O)cc2F)s1] 5-(4-(4-(2,6-difluorobenzyl)-5-oxo-4,5-dihydro-1H-1,2,4-triazol-1-yl)- 2-fluorophenoxy)-2-methylthiazole-4-carboxamide | white solid, yield: 41% | ¹H NMR (400 MHZ, CD₃OD) δ 8.02 (s, 1H), 7.91 (dd, J = 12.3, 2.4 Hz, 1H), 7.78 (d, J = 8.9 Hz, 1H), 7.50-7.41 (m, 1H), 7.35 (t, J = 8.9 Hz, 1H), 7.06 (t, J = 8.1 Hz, 2H), 5.04 (s, 2H), 2.58 (s, 3H). | 462.1 |
| Example 101 Method 20 | +++ | 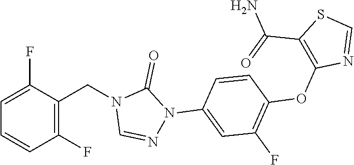 [SMILES: NC(=O)c1scnc1Oc1ccc(-n2ncn(Cc3c(F)cccc3F)c2=O)cc1F] 4-(4-(4-(2,6-difluorobenzyl)-5-oxo-4,5-dihydro-1H-1,2,4- triazol-1-yl)-2-fluorophenoxy)thiazole-5-carboxamide | white solid, yield: 8.1% | ¹H NMR (400 MHZ, CDCl₃) δ 8.57 (s, 1H), 7.98 (dd, J = 12.0, 2.4 Hz, 1H), 7.88 (d, J = 9.0 Hz, 1H), 7.59 (s, 1H), 7.42-7.35 (m, 2H), 7.00 (d, J = 7.7 Hz, 3H), 4.98 (s, 2H). | 448.0 |
| Example 102 Method 20 | ++++ | 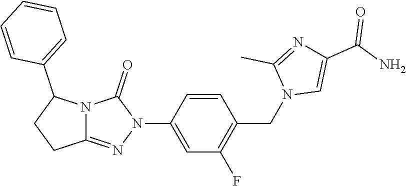 [SMILES: Cc1nc(C(N)=O)cn1Cc1ccc(-n2nc3n(c2=O)C(c2ccccc2)CC3)cc1F] 1-(2-fluoro-4-(3-oxo-5-phenyl-6,7-dihydro-3H-pyrrolo[2,1-c][1,2,4] triazol-2(5H)-yl)benzyl)-2-methyl-1H-imidazole-4-carboxamide | white solid, yield: 5.2% | ¹H NMR (400 MHZ, CDCl₃) δ 7.89 (m, 2H), 7.58 (s, 1H), 7.39 (m, 3H), 7.21 (m, 3H), 5.28 (s,1H), 5.14 (s, 2H), 3.02 (m,4H), 2.70 (s, 3H). | 433.1 |
| Example 103 Method 20 | +++ | 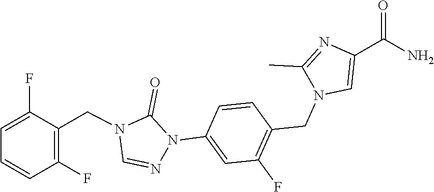 [SMILES: Cc1nc(C(N)=O)cn1Cc1ccc(-n2ncn(Cc3c(F)cccc3F)c2=O)cc1F] 1-(4-(4-(2,6-difluorobenzyl)-5-oxo-4,5-dihydro-1H-1,2,4-triazol-1-yl)-2- fluorobenzyl)-2-methyl-1H-imidazole-4-carboxamide | white solid, yield: 10.6% | ¹H NMR (400 MHZ, CDCl₃) δ 7.90 (m, 2H), 7.58 (d, J = 20.0 Hz, 2H), 7.41-7.33 (m, 1H), 7.16 (m, 1H), 7.01-6.96 (m, 2H), 5.12 (s, 2H), 4.97 (s, 2H), 2.59 (s, 3H). | 443.1 |
| Example 104 Method 20 | ++++ | 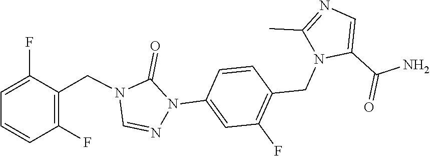 [SMILES: Cc1ncc(C(N)=O)n1Cc1ccc(-n2ncn(Cc3c(F)cccc3F)c2=O)cc1F] 1-(4-(4-(2,6-difluorobenzyl)-5-oxo-4,5-dihydro-1H-1,2,4-triazol-1-yl)- 2-fluorobenzyl)-2-methyl-1H-imidazole-5-carboxamide | white solid, yield: 2.4% | ¹H NMR (400 MHZ, CDCl₃) δ 8.17 (s, 1H), 7.84 (d, J = 11.9 Hz, 1H), 7.73 (d, J = 8.2 Hz, 1H), 7.57 (s, 1H), 7.36 (m, 1H), 7.04 (d, J = 8.0 Hz, 1H), 6.97 (d, J = 8.1 Hz, 2H), 5.72 (s, 2H), 4.96 (s, | 443.1 |
| | | | | 2H), 2.53 (s, 3H). | |

### Example 105: Method 21

### Methyl

### 5-(4-(4-(2,6-difluorobenzyl)-5-oxo-4,5-dihydro-1H-1,2,4-triazol-1-yl)-2-fluorophenoxy)thiazole-2-carboxylate

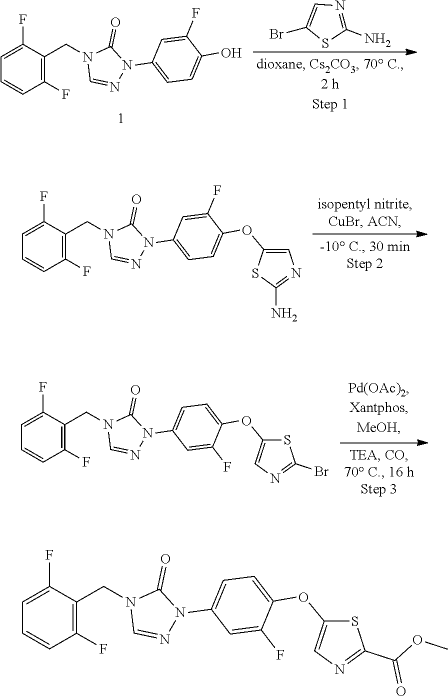 [SMILES: O=c1n(Cc2c(F)cccc2F)cnn1-c1ccc(O)c(F)c1 ; Nc1ncc(Br)s1 ; Nc1ncc(Oc2ccc(-n3ncn(Cc4c(F)cccc4F)c3=O)cc2F)s1 ; O=c1n(Cc2c(F)cccc2F)cnn1-c1ccc(Oc2cnc(Br)s2)c(F)c1 ; COC(=O)c1ncc(Oc2ccc(-n3ncn(Cc4c(F)cccc4F)c3=O)cc2F)s1]

### Step 1: 2-(4-((2-aminothiazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(3-fluoro-4-hydroxyphenyl)-2,4-dihydro-3H-1,2,4-triazol-3-one (5.0 g, 15.56 mmol) and 5-bromothiazol-2-amine (3.1 g, 17.31 mmol) in 1,4-dioxane (50 mL) were added Cs₂CO₃ (12.7 g, 38.98 mmol) at rt. The resulted mixture was stirred for additional 1 h at 70° C. The mixture was allowed to cool down to rt and concentrated under pressure 50° C. The resulted mixture was diluted with water (100 mL). The resulted mixture was extracted with EtOAc (3×50 mL). The combined Organic layers were washed with brine (100 mL), dried over anhydrous Na₂SO₄, and after filtration, the filtrate was concentrated under reduced pressure. The residue was purified by silica gel column chromatography, eluted with (CH₂Cl₂/MeOH) (1:1) to afford 2-(4-((2-aminothiazol-5-yl)oxy)-3-fluorophenyl)-4-(2,6-difluorobenzyl)-2,4-dihydro-3H-1,2,4-triazol-3-one (2.2 g, 33.48% yield) as a yellow solid. MS (m/z): 419.9 [M+H]⁺.

### Step 2: 2-(4-((2-bromothiazol-5-yl)oxy)-3-fluorophenyl)-4-(2,6-difluorobenzyl)-2,4-dihydro-3H-1,2,4-triazol-3-one

To a stirred solution of 2-(4-((2-aminothiazol-5-yl)oxy)-3-fluorophenyl)-4-(2,6-difluorobenzyl)-2,4-dihydro-3H-1,2,4-triazol-3-one (450 mg, 1.07 mmol) and Isoamyl nitrite (151 mg, 1.29 mmol) in ACN (5 mL) were added CuBr₂ (287 mg, 1.29 mmol) at −10° C. The resulted mixture was stirred for additional 1 h at −10° C. The mixture was allowed to warm to rt. The resulted mixture was diluted with water (30 mL). The resulted mixture was extracted with EtOAc (30 mL). The combined Organic layers were washed with brine (60 mL), dried over anhydrous Na₂SO₄, and after filtration, the filtrate was concentrated under reduced pressure. The residue was purified by silica gel column chromatography, eluted with (DCM/MeOH) (20:1) to afford 2-(4-((2-bromothiazol-5-yl)oxy)-3-fluorophenyl)-4-(2,6-difluorobenzyl)-2,4-dihydro-3H-1,2,4-triazol-3-one (70 mg, 12.15% yield) as a yellow oil. MS (m/z): 482.7 [M+H]⁺.

### Step 3: methyl 5-(4-(4-(2,6-difluorobenzyl)-5-oxo-4,5-dihydro-1H-1,2,4-triazol-1-yl)-2-fluorophenoxy)thiazole-2-carboxylate

To a stirred solution of 2-(4-((2-bromothiazol-5-yl)oxy)-3-fluorophenyl)-4-(2,6-difluorobenzyl)-2,4-dihydro-3H-1,2,4-triazol-3-one (60 mg, 0.12 mmol), Pd(OAc)₂ (6 mg, 0.02 mmol) and Xantphos (22 mg 0.04 mmol) in MeOH (3 mL) was added TEA (126 mg, 0.12 mmol) at rt. The resulted mixture was stirred for additional 16 h at 70′C. The solution was filtered, and the filtrate was collected. The reaction mixture was concentrated under reduced pressure at 40′C. The resulted mixture was extracted with EtOAc (3×15 mL). The combined Organic layers were washed with brine (30 mL), dried over anhydrous Na₂SO₄, and after filtration, the filtrate was concentrated under reduced pressure. The residue was purified by silica gel column chromatography, eluted with (PE:EtOAc) (1:1) to afford methyl 5-(4-(4-(2,6-difluorobenzyl)-5-oxo-4,5-dihydro-1H-1,2,4-triazol-1-yl)-2-fluorophenoxy)thiazol e-2-carboxylate (19 mg, 33.06% yield) as a white solid. LC-MS (m/z) 462.9 [M+H]⁺. ¹H NMR (400 MHz, CDCl₃) δ 7.98 (dd, J=12.0, 2.5 Hz, 1H), 7.85 (ddd, J=9.0, 2.5, 1.6 Hz, 1H), 7.61 (s, 1H), 7.45 (s, 1H), 7.42-7.34 (m, 1H), 7.28 (m, J=8.8 Hz, 1H), 7.03-6.95 (m, 2H), 4.98 (s, 2H), 3.97 (s, 3H).

Examples (Compounds) 106-108 were synthesized using a similar method to that used in Example 105.

| Com- | | | Appear- | | |
|---|---|---|---|---|---|
| pound | | | ance | | |
| No. | Ac- | | and | | MS(m/z) |
| Method | tivity | Structure and Name | Yield | ¹H NMR Data | [M + H]⁺ |
| Exam- ple 105 Method 21 | +++ | 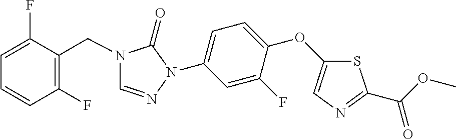 [SMILES: COC(=O)c1ncc(Oc2ccc(-n3ncn(Cc4c(F)cccc4F)c3=O)cc2F)s1] | white solid, yield: 33.06% | ¹H NMR (400 MHz, CDCl₃) δ 7.98 (dd, J = 12.0, 2.5 Hz, 1H), 7.85 (ddd, J = 9.0, 2.5, 1.6 Hz, 1H), 7.61 (s, 1H), 7.45 (s, 1H), 7.42-7.34 (m, 1H), 7.28 (m, J = 8.8 Hz, 1H), 7.03-6.95 (m, 2H), 4.98 (s, 2H), 3.97 (s, 3H). | 462.9 |
| | | methyl | | | |
| | | 5-(4-(4-(2,6-difluorobenzyl)-5- | | | |
| | | oxo-4,5-dihydro-1H-1,2,4- | | | |
| | | triazol-1-yl)-2-fluorophenoxy) | | | |
| | | thiazole-2-carboxylate | | | |
| Exam- ple 106 Method 21 | ++++ | 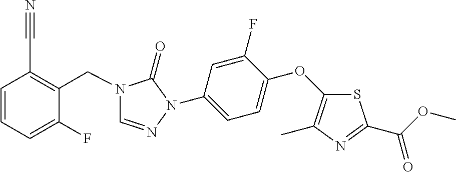 [SMILES: COC(=O)c1nc(C)c(Oc2ccc(-n3ncn(Cc4c(F)cccc4C#N)c3=O)cc2F)s1] | white solid, yield: 12.5% | ¹H NMR (400 MHz, DMSO-d₆) δ 8.42 (d, J = 1.2 Hz, 1H), 7.91 (dd, J = 12.4, 2.8 Hz, 1H), 7.78 (dd, J = 6.8, 2.0 Hz, 1H), 7.75-7.70 (m, 1H), 7.70-7.60 (m, 2H), 7.45 (t, J = 9.2 Hz, 1H), 5.13 (s, 2H), 3.87 (s, 3H), 2.34 (s, 3H). | 484.0 |
| | | methyl | | | |
| | | 5-(4-(4-(2-cyano-6-fluorobenzyl)- | | | |
| | | 5-oxo-4,5-dihydro-1H-1,2,4- | | | |
| | | triazol-1-yl)-2-fluorophenoxy)-4- | | | |
| | | methylthiazole-2-carboxylate | | | |
| Exam- ple 107 Method 21 | ++++ | 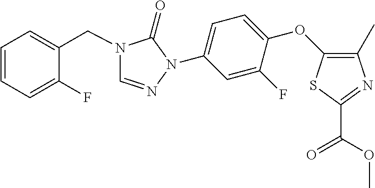 [SMILES: COC(=O)c1nc(C)c(Oc2ccc(-n3ncn(Cc4ccccc4F)c3=O)cc2F)s1] | white solid, yield: 73.2% | ¹H NMR (400 MHz, DMSO-d₆) δ 8.38 (s, 1H), 7.94 (dd, J = 12.6, 2.5 Hz, 1H), 7.76 (ddd, J = 9.0, 2.6, 1.5 Hz, 1H), 7.53-7.37 (m, 3H), 7.33-7.18 (m, 2H), 4.96 (s, 2H), 3.87 (s, 3H), 2.34 (s, 3H). | 459.1 |
| | | methyl | | | |
| | | 5-(2-fluoro-4-(4-(2-fluorobenzyl)- | | | |
| | | 5-oxo-4,5-dihydro-1H-1,2,4- | | | |
| | | triazol- | | | |
| | | 1-yl)phenoxy)-4-methylthiazole- | | | |
| | | 2-carboxylate | | | |
| Exam- ple 108 Method 21 | ++++ | 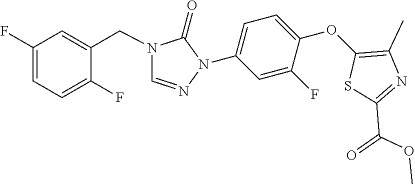 [SMILES: COC(=O)c1nc(C)c(Oc2ccc(-n3ncn(Cc4cc(F)ccc4F)c3=O)cc2F)s1] | white solid, yield: 74.9% | ¹H NMR (400 MHz, DMSO-d₆) δ 8.38 (s, 1H), 7.94 (dd, J = 12.6, 2.5 Hz, 1H), 7.76 (ddd, J = 9.1, 2.6, 1.4 Hz, 1H), 7.47 (t, J = 9.1 Hz, 1H), 7.38-7.19 (m, 3H), 4.95 (s, 2H), 3.87 (s, 3H), 2.34 (s, 3H). | 477.1 |
| | | methyl | | | |
| | | 5-(4-(4-(2,5-difluorobenzyl)-5- | | | |
| | | oxo-4,5-dihydro-1H-1,2,4-triazol- | | | |
| | | 1-yl)- | | | |
| | | 2-fluorophenoxy)-4- | | | |
| | | methylthiazole-2-carboxylate | | | |

### Example 109: Method 22

### 2-(4-((2-(3-amino-1-fluorocyclobutyl)-4-methylthiazol-5-yl)oxy)-3-fluorophenyl)-4-(2,6-difluorobenzyl)-2,4-dihydro-3H-1,2,4-triazol-3-one

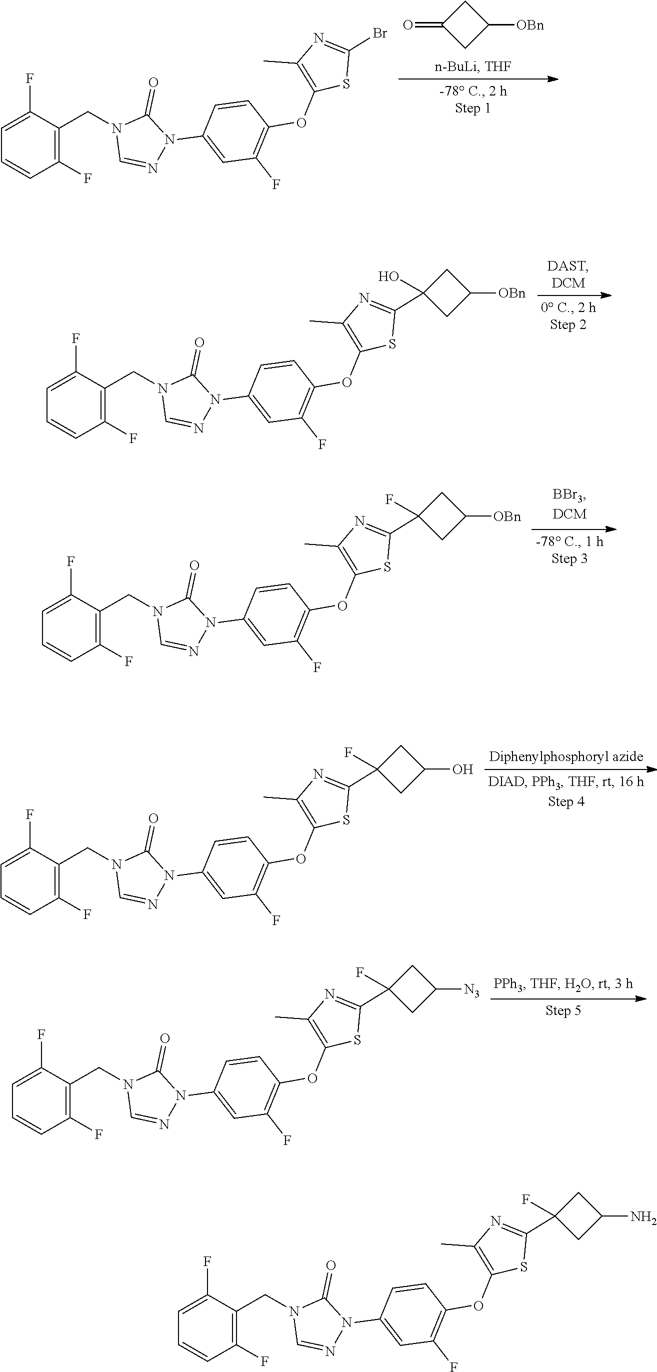

### Step 1: 2-(4-((2-(3-(benzyloxy)-1-hydroxycyclobutyl)-4-methylthiazol-5-yl)oxy)-3-fluorophenyl)-4-(2,6-difluorobenzyl)-2,4-dihydro-3H-1,2,4-triazol-3-one

To a stirred solution of 2-(4-((2-bromo-4-methylthiazol-5-yl)oxy)-3-fluorophenyl)-4-(2,6-difluorobenzyl)-2,4-dihydro-3H-1,2,4-triazol-3-one (5 g, 10.1 mmol) in THF (100 mL) at −78° C., under N₂ atmosphere, was added n-BuLi in a THF solution (4.1 mL, 2.5M, 10.1 mmol). Then the mixture solution was stirred at −78° C. for 20 min. To the reaction mixture, was added 3-(benzyloxy) cyclobutan-1-one (2.7 g, 15.2 mmol), and the resulted mixture solution was stirred at −78° C. for 100 min. The reaction was quenched with saturation NH₄Cl solution and was diluted with EtOAc (100 mL), and then the resulted solution was washed with water (30 mL) and brine (30 mL), dried over anhydrous Na₂SO₄, and after filtration, the filtrate was concentrated under reduced pressure. The residue was purified by silica gel column chromatography, eluted with (PE/EtOAc) (2:1) to afford 2-(4-((2-(3-(benzyloxy)-1-hydroxycyclobutyl)-4-methylthiazol-5-yl)oxy)-3-fluorophenyl)-4-(2,6-difluorobenzyl)-2,4-dihydro-3H-1,2,4-triazol-3-one (2.4 g, 40.0% yield) as a yellow solid. MS (m/z): 595.1 [M+H]⁺. ¹H NMR (400 MHz, CDCl₃) δ 7.90 (dd, J=12.0, 2.4 Hz, 1H), 7.72 (d, J=8.8 Hz, 1H), 7.57 (s, 1H), 7.44-7.27 (m, 6H), 7.09-6.92 (m, 3H), 4.97 (s, 2H), 4.48 (s, 2H), 4.13 (p, J=6.4 Hz, 1H), 3.06-2.96 (m, 2H), 2.53-2.43 (m, 2H), 2.29 (s, 3H).

### Step 2: 2-(4-((2-(3-(benzyloxy)-1-fluorocyclobutyl)-4-methylthiazol-5-yl)oxy)-3-fluorophenyl)-4-(2,6-difluorobenzyl)-2,4-dihydro-3H-1,2,4-triazol-3-one

To a stirred solution of 2-(4-((2-(3-(benzyloxy)-1-hydroxycyclobutyl)-4-methylthiazol-5-yl)oxy)-3-fluorophenyl)-4-(2,6-difluorobenzyl)-2,4-dihydro-3H-1,2,4-triazol-3-one (2.4 g, 4.04 mmol) in DCM (30 mL), was added Diethylamino sulfur trifluoride (0.78 g, 4.85 mmol) and the mixture solution was stirred at rt for 1 h. The resulted mixture was diluted with water (20 mL). The resulted mixture was extracted with DCM (3×20 mL). The combined organic layers were washed with brine (10 mL), dried over anhydrous Na₂SO₄, and after filtration, the filtrate was concentrated under reduced pressure. The residue was purified by silica gel column chromatography, eluted with (PE/EtOAc) (2:1) to afford 2-(4-((2-(3-(benzyloxy)-1-fluorocyclobutyl)-4-methylthiazol-5-yl)oxy)-3-fluorophenyl)-4-(2,6-difluorobenzyl)-2,4-dihydro-3H-1,2,4-triazol-3-one (1.5 g, 61.9% yield) as a white solid. MS (m/z): 597.2 [M+H]⁺.

¹H NMR (400 MHz, CDCl₃) δ 7.90 (dd, J=12.0, 2.4 Hz, 1H), 7.71 (d, J=8.8 Hz, 1H), 7.58 (s, 1H), 7.44-7.28 (m, 6H), 7.06-6.94 (m, 3H), 4.97 (s, 2H), 4.49 (s, 2H), 4.47-4.41 (m, 1H), 2.94-2.66 (m, 4H), 2.31 (s, 3H).

### Step 3: 4-(2,6-difluorobenzyl)-2-(3-fluoro-4-((2-(1-fluoro-3-hydroxycyclobutyl)-4-methylthiazol-5-yl)oxy)phenyl)-2,4-dihydro-3H-1,2,4-triazol-3-one

To a stirred solution of 2-(4-((2-(3-(benzyloxy)-1-fluorocyclobutyl)-4-methylthiazol-5-yl)oxy)-3-fluorophenyl)-4-(2,6-difluorobenzyl)-2,4-dihydro-3H-1,2,4-triazol-3-one (1.5 g, 2.5 mmol) in DCM (20 mL) at −78° C., under N₂ atmosphere, was added BBr₃ in DCM solution (3.0 mL, 1M, 2.0 mmol). Then the mixture solution was stirred at −78° C. for 1h. The reaction was quenched with a saturated NaHCO₃ solution and was diluted with DCM (20 mL), and the resulted solution was washed with water (20 mL) and brine (20 mL), dried over anhydrous Na₂SO₄, and after filtration, the filtrate was concentrated under reduced pressure. The residue was purified by silica gel column chromatography, eluted with (PE/EtOAc) (1:1) to afford 4-(2,6-difluorobenzyl)-2-(3-fluoro-4-((2-(1-fluoro-3-hydroxycyclobutyl)-4-methylthiazol-5-yl)oxy)phenyl)-2,4-dihydro-3H-1,2,4-triazol-3-one (600 mg, 47.4% yield) as a white solid. MS (m/z): 507.2 [M+H]⁺.

¹H NMR (400 MHz, CDCl₃) δ 7.98-7.87 (m, 1H), 7.73 (t, J=10.8 Hz, 1H), 7.58 (d, J=2.8 Hz, 1H), 7.44-7.32 (m, 1H), 7.12-6.95 (m, 3H), 4.97 (s, 2H), 4.60-4.29 (m, 1H), 3.25-2.95 (m, 2H), 2.88-2.57 (m, 2H), 2.32 (d, J=26.8 Hz, 3H).

### Step 4: 2-(4-((2-(3-azido-1-fluorocyclobutyl)-4-methylthiazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(3-fluoro-4-((2-(1-fluoro-3-hydroxycyclobutyl)-4-methylthiazol-5-yl)oxy)phenyl)-2,4-dihydro-3H-1,2,4-triazol-3-one (200 mg, 0.40 mmol) in THF (10 mL) at 0° C., under N₂ atmosphere, wad successively added PPh₃ (157 mg, 0.60 mmol) and DIAD (121 mg, 0.60 mmol). Then the mixture solution was stirred at 0° C. for 15 min. The reaction mixture was then added Diphenyl- phosphoryl azide (165 mg, 0.60 mmol), and stirred at rt for 16 h. The reaction was quenched with a saturated NaHCO₃ solution and was diluted with EtOAc (10 mL), the resulted solution was washed with water (10 mL) and brine (10 mL), dried over anhydrous Na₂SO₄, and after filtration, the filtrate was concentrated under reduced pressure. The residue was purified by silica gel column chromatography, eluted with (PE/EtOAc) (2:1) to afford 2-(4-((2-(3-azido-1-fluorocyclobutyl)-4-methylthiazol-5-yl)oxy)-3-fluorophenyl)-4-(2,6-difluorobenzyl)-2,4-dihydro-3H-1,2,4-triazol-3-one(130 mg, 61.2% yield) as a white solid. MS (m/z): 531.2[M+H]⁺.

### Step 5: 2-(4-((2-(3-amino-1-fluorocyclobutyl)-4-methylthiazol-5-yl)oxy)-3-fluorophenyl)-4-(2,6-difluorobenzyl)-2,4-dihydro-3H-1,2,4-triazol-3-one

To a stirred solution of 2-(4-((2-(3-azido-1-fluorocyclobutyl)-4-methylthiazol-5-yl)oxy)-3-fluorophenyl)-4-(2,6-difluorobenzyl)-2,4-dihydro-3H-1,2,4-triazol-3-one (110 mg, 0.22 mmol) in THF/H₂O mixture solvent (10/1, 10 mL), under N₂ atmosphere, was added PPh₃ (86 mg, 0.33 mmol). The mixture solution was stirred at rt for 3 h. The resulted mixture was diluted with EtOAc (10 mL), the resulted solution was washed with water (10 mL) and brine (10 mL), dried over anhydrous Na₂SO₄, and after filtration, the filtrate was concentrated under reduced pressure. The residue was purified by silica gel column chromatography, eluted with (DCM/MeOH) (15:1) to afford the titled compound as a white solid (60 mg, 54.1% yield) as a white solid. MS (m/z): 506.2 [M+H]⁺. ¹H NMR (400 MHz, CDCl₃) δ 7.90 (d, J=12.4 Hz, 1H), 7.71 (d, J=8.8 Hz, 1H), 7.57 (s, 1H), 7.44-7.30 (m, 1H), 7.06-6.92 (m, 3H), 4.97 (s, 2H), 3.51-3,38 (m, 1H), 3.18-2.81 (m, 2H), 2.65-2.32 (m, 2H), 2.29 (s, 3H).

Examples (Compounds) 110-112 were synthesized using a similar method to that used in Example 109.

| Com- | | | Appear- | | MS |
|---|---|---|---|---|---|
| pound | | | ance | | (m/z) |
| No. | Ac- | | and | | [M + |
| Method | tivity | Structure and Name | Yield | ¹H NMR Data | H]⁺ |
| Exam- ple 109 Method 22 | ++++ | 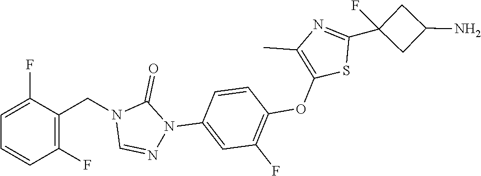 [SMILES: Cc1nc(C2(F)CC(N)C2)sc1Oc1ccc(-n2ncn(Cc3c(F)cccc3F)c2=O)cc1F] | white solid, yield: 54.1%) | ¹H NMR (400 MHz, CDCl₃) δ 7.90 (d, J = 12.4 Hz, 1H), 7.71 (d, J = 8.8 Hz, 1H), 7.57 (s, 1H), 7.44-7.30 (m, 1H), 7.06-6.92 (m, 3H), 4.97 (s, 2H), 3.51-3,38 (m, 1H), 3.18-2.81 (m, 2H), 2.65-2.32 (m, 2H), 2.29 (s, 3H). | 506.2 |
| | | 2-(4-((2-(3-amino-1-fluorocyclobutyl)- | | | |
| | | 4-methylthiazol-5-yl)oxy)-3- | | | |
| | | fluorophenyl)-4-(2,6-difluorobenzyl)- | | | |
| | | 2,4-dihydro-3H-1,2,4-triazol- | | | |
| | | 3-one | | | |
| Exam- ple 110 Method 22 | ++++ | 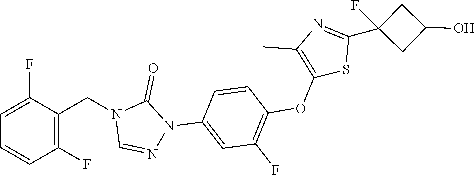 [SMILES: Cc1nc(C2(F)CC(O)C2)sc1Oc1ccc(-n2ncn(Cc3c(F)cccc3F)c2=O)cc1F] | white solid, yield: 47.4% | ¹H NMR (400 MHz, CDCl₃) δ 7.98- 7.87 (m, 1H), 7.73 (t, J = 10.8 Hz, 1H), 7.58 (d, J = 2.8 Hz, 1H), 7.44- 7.32 (m, 1H), 7.12-6.95 (m, 3H), 4.97 (s, 2H), 4.60-4.29 (m, 1H), 3.25-2.95 (m, 2H), 2.88-2.57 (m, 2H), 2.32 (d, J = 26.8 Hz, 3H). | 507.2 |
| | | 4-(2,6-difluorobenzyl)-2-(3-fluoro- | | | |
| | | 4-((2-(1-fluoro-3-hydroxycyclo- | | | |
| | | butyl)-4-methylthiazol-5-yl)oxy) | | | |
| | | phenyl)-2,4-dihydro-3H-1,2,4- | | | |
| | | triazol-3-one | | | |
| Exam- ple 111 Method 22 | ++++ | 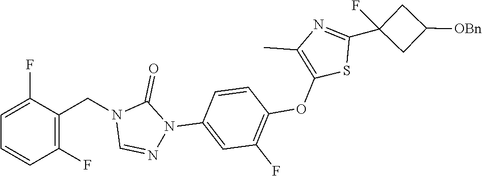 [SMILES: Cc1nc(C2(F)CC(OCc3ccccc3)C2)sc1Oc1ccc(-n2ncn(Cc3c(F)cccc3F)c2=O)cc1F] | white solid, yield: 61.9% | ¹H NMR (400 MHz, CDCl₃) δ 7.90 (dd, J = 12.0, 2.4 Hz, 1H), 7.71 (d, J = 8.8 Hz, 1H), 7.58 (s, 1H), 7.44- 7.28 (m, 6H), 7.06-6.94 (m, 3H), 4.97 (s, 2H), 4.49 (s, 2H), 4.47-4.41 (m, 1H), 2.94-2.66 (m, 4H), 2.31 (s, 3H). | 597.2 |
| | | 2-(4-((2-(3-(benzyloxy)-1-fluoro- | | | |
| | | cyclobutyl)-4-methylthiazol-5-yl) | | | |
| | | oxy)-3-fluorophenyl)-4-(2,6-difluoro- | | | |
| | | benzyl)-2,4-dihydro-3H-1,2,4- | | | |
| | | triazol-3-one | | | |
| Exam- ple 112 Method 22 | +++ | 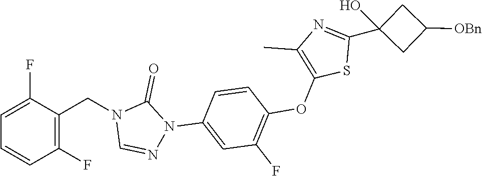 [SMILES: Cc1nc(C2(O)CC(OCc3ccccc3)C2)sc1Oc1ccc(-n2ncn(Cc3c(F)cccc3F)c2=O)cc1F] | white solid, yield: 40% | ¹H NMR (400 MHz, CDCl₃) δ 7.90 (dd, J = 12.0, 2.4 Hz, 1H), 7.72 (d, J = 8.8 Hz, 1H), 7.57 (s, 1H), 7.44- 7.27 (m, 6H), 7.09-6.92 (m, 3H), 4.97 (s, 2H), 4.48 (s, 2H), 4.13 (p, J = 6.4 Hz, 1H), 3.06-2.96 (m, 2H), 2.53-2.43 (m, 2H), 2.29 (s, 3H). | 595.1 |
| | | 2-(4-((2-(3-(benzyloxy)-1-hydroxy- | | | |
| | | cyclobutyl)-4-methylthiazol-5-yl) | | | |
| | | oxy)-3-fluorophenyl)-4-(2,6- | | | |
| | | difluorobenzyl)-2,4-dihydro-3H- | | | |
| | | 1,2,4-triazol-3-one | | | |

### Example 113: Method 23

### 3-(5-(4-(4-(2,6-difluorobenzyl)-5-oxo-4,5-dihydro-1H-1,2,4-triazol-1-yl)-2-fluorophenoxy)-4-methylthiazol-2-yl) cyclobutane-1-carboxamide

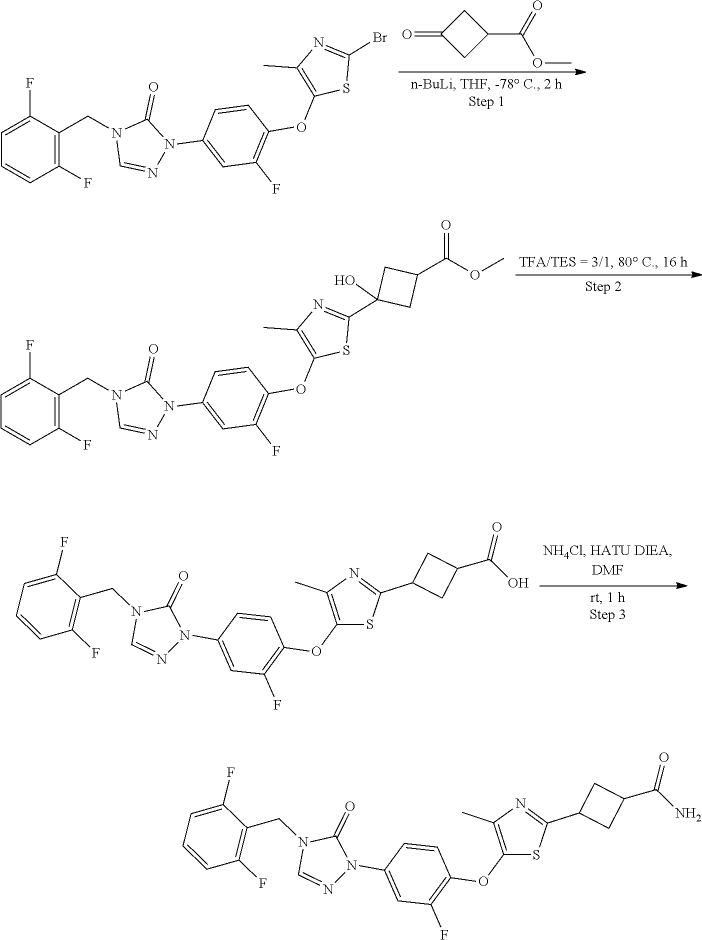

### Step 1: methyl 3-(5-(4-(4-(2,6-difluorobenzyl)-5-oxo-4,5-dihydro-1H-1,2,4-triazol-1-yl)-2-fluorophenoxy)-4-methylthiazol-2-yl)-3-hydroxycyclobutane-1-carboxylate

To a stirred solution of 2-(4-((2-bromo-4-methylthiazol-5-yl)oxy)-3-fluorophenyl)-4-(2,6-difluorobenzyl)-2,4-dihydro-3H-1,2,4-triazol-3-one (800 mg, 1.61 mmol) in THF (10 mL) at −78° C., under N₂ atmosphere, was added n-BuLi in a THF solution (0.64 mL, 2.5M, 1.61 mmol). Then the mixture solution was stirred at −78° C. for 20 min. The reaction mixture was added methyl 3-oxocyclobutane-1-carboxylate (309 mg, 2.42 mmol), and stirred at −78° C. for 100 min. The reaction was quenched with a saturated NH₄Cl solution and was diluted with EtOAc (20 mL), the resulted solution was washed with water (10 mL) and brine (10 mL), dried over anhydrous Na₂SO₄, and after filtration, the filtrate was concentrated under reduced pressure. The residue was purified by silica gel column chromatography, eluted with (PE/EtOAc) (2:1) to afford methyl 3-(5-(4-(4-(2,6-difluorobenzyl)-5-oxo-4,5-dihydro-1H-1,2,4-triazol-1-yl)-2-fluorophenoxy)-4-methylthiazol-2-yl)-3-hydroxycyclobutane-1-carboxylate (350 mg, 39.8% yield) as a yellow solid. MS (m/z): 547.1[M+H]⁺.

### Step 2: 3-(5-(4-(4-(2,6-difluorobenzyl)-5-oxo-4,5-dihydro-1H-1,2,4-triazol-1-yl)-2-fluorophenoxy)-4-methylthiazol-2-yl) cyclobutane-1-carboxylic acid

A solution of methyl 3-(5-(4-(4-(2,6-difluorobenzyl)-5-oxo-4,5-dihydro-1H-1,2,4-triazol-1-yl)-2-fluorophenoxy)-4-methylthiazol-2-yl)-3-hydroxycyclobutane-1-carboxylate (150 mg, 0.27 mmol) in TFA/TES (3/1, 5 mL) was stirred at 80° C. for O/N (overnight). The resulted mixture was diluted with a saturated NaHCO₃ solution (10 mL). The resulted mixture was extracted with DCM (3×10 mL). The combined Organic layers were washed with brine (10 mL), dried over anhydrous Na₂SO₄, and after filtration, the filtrate was concentrated under reduced pressure. The residue was purified by silica gel column chromatography, eluted with (DCM/MeOH) (15:1) to afford 3-(5-(4-(4-(2,6-difluorobenzyl)-5-oxo-4,5-dihydro-1H-1,2,4-triazol-1-yl)-2-fluorophenoxy) -4-methylthiazol-2-yl) cyclobutane-1-carboxylic acid (80 mg, 57.4% yield) as a white solid.MS (m/z): 517.2 [M+H]⁺.

### Step 3: 3-(5-(4-(4-(2,6-difluorobenzyl)-5-oxo-4,5-dihydro-1H-1,2,4-triazol-1-yl)-2-fluorophenoxy)-4-methylthiazol-2-yl) cyclobutane-1-carboxamide

A solution of 3-(5-(4-(4-(2,6-difluorobenzyl)-5-oxo-4,5-dihydro-1H-1,2,4-triazol-1-yl)-2-fluorophenoxy) -4-methylthiazol-2-yl) cyclobutane-1-carboxylic acid (80 mg, 0.16 mmol), Ammonium chloride (13 mg, 0.24 mmol), HATU (93 mg, 0.16 mmol) and DIEA (31 mg, 0.24 mmol) in DMF (5 mL) was under N₂ atmosphere and stirred at rt for 1 h. The resulted mixture was diluted with EtOAc (10 mL), the solution was washed with water (3×5 mL) and brine (5 mL), dried over anhydrous Na₂SO₄, and after filtration, the filtrate was concentrated under reduced pressure. The residue was purified by silica gel column chromatography, eluted with (DCM/MeOH=10:1) to afford the titled compound as a white solid (25.5 mg, 30.9% yield) as a white solid. MS (m/z): 516.2 [M+H]⁺. ¹H NMR (400 MHz, CDCl₃) δ 7.97 (t, J=13.2 Hz, 1H), 7.87-7.76 (m, 1H), 7.60 (d, J=4.4 Hz, 1H), 7.46-7.28 (m, 1H), 7.19-6.93 (m, 3H), 4.98 (s, 2H), 4.03 (s, 1H), 3.38-3.09 (m, 1H), 2.98-2.72 (m, 4H), 2.50 (d, J=14.0 Hz, 3H).

Examples (Compounds) 114-115 were synthesized using a similar method to that used in Example 113.

| Com- | | | Ap- | | |
|---|---|---|---|---|---|
| pound | | | pear- | | MS |
| No. | | | ance | | (m/z) |
| Meth- | Ac- | | and | | [M + |
| od | tivity | Structure and Name | Yield | ¹H NMR Data | H]⁺ |
| Ex- am- ple 113 Meth- od 23 | ++++ | 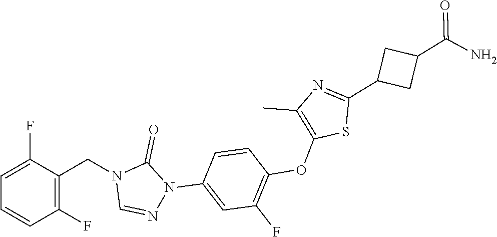 [SMILES: Cc1nc(C2CC(C(N)=O)C2)sc1Oc1ccc(-n2ncn(Cc3c(F)cccc3F)c2=O)cc1F] | white solid, yield: 30.9% | ¹H NMR (400 MHz, CDCl₃) δ 7.97 (t, J = 13.2 Hz, 1H), 7.87-7.76 (m, 1H), 7.60 (d, J = 4.4 Hz, 1H), 7.46-7.28 (m, 1H), 7.19-6.93 (m, 3H), 4.98 (s, 2H), 4.03 (s, 1H), 3.38-3.09 (m, 1H), 2.98-2.72 (m, 4H), 2.50 (d, J = 14.0 Hz, 3H). | 516.2 |
| | | 3-(5-(4-(4-(2,6-difluorobenzyl)-5- | | | |
| | | oxo-4,5-dihydro-1H-1,2,4-triazol- | | | |
| | | 1-yl)-2-fluorophenoxy)-4-methyl- | | | |
| | | thiazol-2-yl)cyclobutane-1- | | | |
| | | carboxamide | | | |
| Exam- ple 114 Meth- od 23 single un- known stereo- isomer | ++++ | 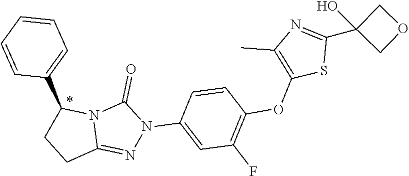 [SMILES: Cc1nc(C2(O)COC2)sc1Oc1ccc(-n2nc3n(c2=O)[C@H](c2ccccc2)CC3)cc1F] | white solid, yield: | ¹H NMR (400 MHz, CDCl₃) δ 7.89 (dd, J = 12.4, 2.0 Hz, 1H), 7.71 (d, J = 9.2 Hz, 1H), 7.45-7.25 (m, 5H), 7.06-6.99 (m, 1H), 5.32-5.34 (m, 1H), 4.92 (d, J = 6.8 Hz, 2H), 4.86 (d, J = 6.8 Hz, 2H), 3.15-2.86 (m, 4H), 2.33 (s, 3H). | 481.2 |
| | | (S)-2-(3-fluoro-4-((2-(3-hydroxy | | | |
| | | oxetan-3-yl)-4-methylthiazol-5-yl) | | | |
| | | oxy)phenyl)-5-phenyl-2,5,6,7- | | | |
| | | tetrahydro-3H-pyrrolo[2,1-c][1,2,4] | | | |
| | | triazol-3-one | | | |
| Ex- am- ple 115 Meth- od 23 | ++ | 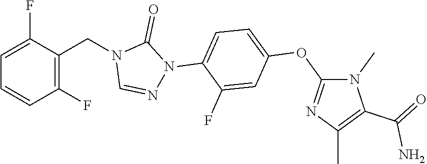 [SMILES: Cc1nc(Oc2ccc(-n3ncn(Cc4c(F)cccc4F)c3=O)c(F)c2)n(C)c1C(N)=O] | white solid, yield: 40.1% | ¹H NMR (400 MHz, DMSO-d₆) δ 8.27 (s, 1H), 7.56 (t, J = 8.7 Hz, 1H), 7.53-7.42 (m, 2H), 7.24 (br, 2H), 7.21-7.12 (m, 3H), 4.97 (s, 2H), 3.59 (s, 3H), 2.23 (s, 3H). | 459.1 |
| | | 2-(4-(4-(2,6-difluorobenzyl)-5-oxo- | | | |
| | | 4,5-dihydro-1H-1,2,4-triazol-1- | | | |
| | | yl)-3-fluorophenoxy)-1,4-dimethyl- | | | |
| | | 1H-imidazole-5-carboxamide | | | |

### Example 116: Method 24

### 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetamide

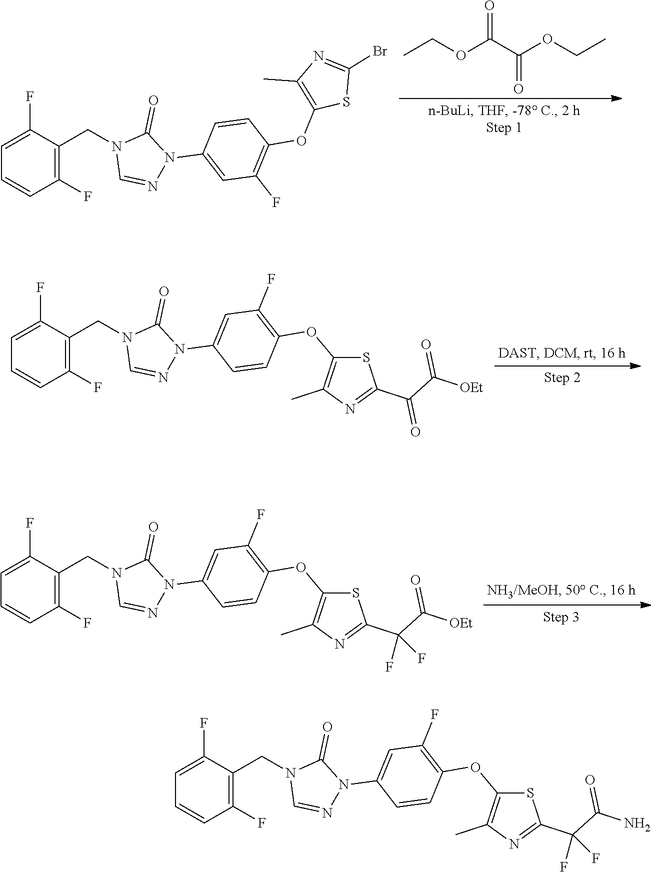

### Step 1: ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2-oxoacetate

To a stirred solution of 2-(4-((2-bromo-4-methylthiazol-5-yl)oxy)-3-fluorophenyl)-4-(2,6-difluorobenzyl)-2,4-dihydro-3H-1,2,4-triazol-3-one (500 mg, 1.01 mmol) in THF (10 mL) at −78° C., under N₂ atmosphere, added n-BuLi in THF solution (0.41 mL, 2.5M, 1.01 mmol). Then the mixture solution was stirred at −78° C. for 20 min. The reaction mixture was added diethyl oxalate (221 mg, 1.52 mmol), and stirred at −78° C. for 100 min. The reaction was quenched with saturated NH₄Cl solution and was diluted with EtOAc (10 mL), the resulted solution was washed with water (10 mL) and brine (10 mL), dried over anhydrous Na₂SO₄, and after filtration, the filtrate was concentrated under reduced pressure. The residue was purified by silica gel column chromatography, eluted with (PE/EtOAc) (2:1) to afford ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2-oxoacetate (200 mg, 38.2% yield) as a white solid. MS (m/z): 519.1 [M+H]⁺.

### Step 2: ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetate

To a stirred solution of ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2-oxoacetate (150 mg, 0.29 mmol) in DCM (10 mL), was added Diethylamino sulfur trifluoride (140 mg, 0.87 mmol) and the reaction mixture was stirred at rt for 16 h. The resulted mixture was diluted with water (10 mL). The resulted mixture was extracted with DCM (3×10 mL). The combined organic layers were washed with brine (10 mL), dried over anhydrous Na₂SO₄, and after filtration, the filtrate was concentrated under reduced pressure. The residue was purified by silica gel column chromatography, eluted with (PE/EtOAc) (2:1) to afford ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetate (100 mg, 63.9% yield) as a white solid.MS (m/z): 541.2 [M+H]⁺.

### Step 3: 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetamide

A solution of ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetate (50 mg, 0.09 mmol) in NH₃ in MeOH solution (5 mL, 7M) was stirred at 50° C. for 16 h. The resulted solution was allowed to cool down to r.t. and concentrated under reduced pressure. The residue was purified by prep-TLC (DCM/MeOH=20/1) to afford the titled compound as a white solid (25 mg, yield: 54.4%). MS (m/z): 512.2 [M+H]⁺. ¹H NMR (400 MHz, CDCl₃) δ 7.95 (dd, J=12.0, 2.4 Hz, 1H), 7.77 (d, J=9.2 Hz, 1H), 7.59 (s, 1H), 7.45-7.29 (m, 1H), 7.15-6.91 (m, 3H), 4.97 (s, 2H), 2.36 (s, 3H).

Examples (compounds) 117-118 were synthesized using a similar method to that used in Example 116.

| Com- | | | Appear- | | MS |
|---|---|---|---|---|---|
| pound | | | ance | | (m/z) |
| No. | Ac- | | and | | [M + |
| Method | tivity | Structure and Name | Yield | ¹H NMR Data | H]⁺ |
| Exam- ple 116 Method 24 | ++++ | 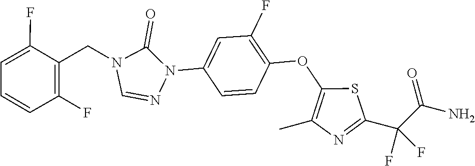 [SMILES: Cc1nc(C(F)(F)C(N)=O)sc1Oc1ccc(-n2ncn(Cc3c(F)cccc3F)c2=O)cc1F] | white solid, yield: 54.4% | ¹H NMR (400 MHz, CDCl₃) δ 7.95 (dd, J = 12.0, 2.4 Hz, 1H), 7.77 (d, J = 9.2 Hz, 1H), 7.59 (s, 1H), 7.45-7.29 (m, 1H), 7.15-6.91 (m, 3H), 4.97 (s, 2H), 2.36 (s, 3H). | 512.2 |
| | | 2-(5-(4-(4-(2,6-difluorobenzyl)-5- | | | |
| | | oxo-4,5-dihydro-1H-1,2,4-triazol- | | | |
| | | 1-yl)-2-fluorophenoxy)-4- | | | |
| | | methylthiazol-2-yl)-2,2- | | | |
| | | difluoroacetamide | | | |
| Exam- ple 117 Method 24 | +++ | 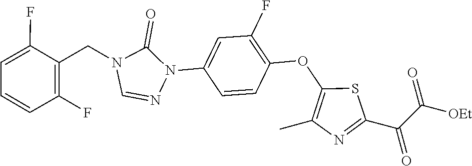 [SMILES: CCOC(=O)C(=O)c1nc(C)c(Oc2ccc(-n3ncn(Cc4c(F)cccc4F)c3=O)cc2F)s1] | whit solid, yield: 38.2% | NA | 519.1 |
| | | ethyl | | | |
| | | 2-(5-(4-(4-(2,6-difluorobenzyl)-5- | | | |
| | | oxo-4,5-dihydro-1H-1,2,4-triazol- | | | |
| | | 1-yl)-2-fluorophenoxy)-4-methyl- | | | |
| | | thiazol-2-yl)-2-oxoacetate | | | |
| Exam- ple 118 Method 24 | +++ | 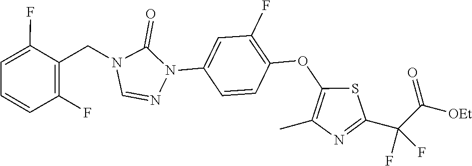 [SMILES: CCOC(=O)C(F)(F)c1nc(C)c(Oc2ccc(-n3ncn(Cc4c(F)cccc4F)c3=O)cc2F)s1] | white solid, yield: 63.9% | NA | 541.2 |
| | | ethyl | | | |
| | | 2-(5-(4-(4-(2,6-difluorobenzyl)-5- | | | |
| | | oxo-4,5-dihydro-1H-1,2,4-triazol- | | | |
| | | 1-yl)-2-fluorophenoxy)-4-methyl- | | | |
| | | thiazol-2-yl)-2,2-difluoroacetate | | | |

### Example 119: Method 25

### 4-(2,6-difluorobenzyl)-2-(3-fluoro-4-((2-(3-hydroxy-3-methylcyclobutyl)-4-methylthiazol-5-yl)oxy)phenyl)-2,4-dihydro-3H-1,2,4-triazol-3-one

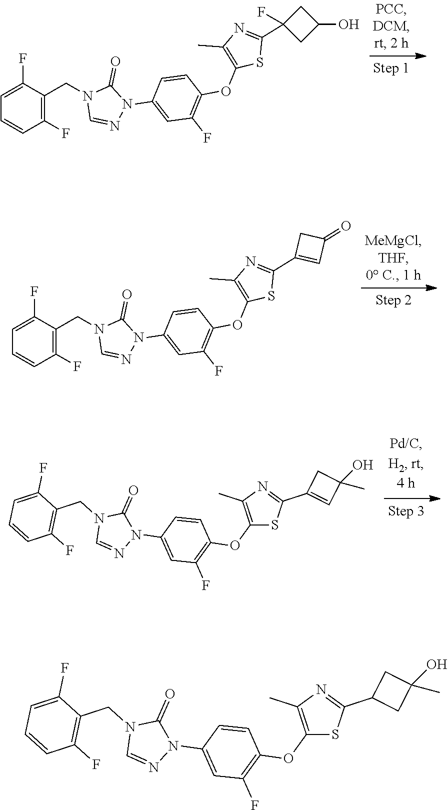 [SMILES: Cc1nc(C2(F)CC(O)C2)sc1Oc1ccc(-n2ncn(Cc3c(F)cccc3F)c2=O)cc1F ; Cc1nc(C2=CC(=O)C2)sc1Oc1ccc(-n2ncn(Cc3c(F)cccc3F)c2=O)cc1F ; Cc1nc(C2=CC(C)(O)C2)sc1Oc1ccc(-n2ncn(Cc3c(F)cccc3F)c2=O)cc1F ; Cc1nc(C2CC(C)(O)C2)sc1Oc1ccc(-n2ncn(Cc3c(F)cccc3F)c2=O)cc1F]

### Step 1: 4-(2,6-difluorobenzyl)-2-(3-fluoro-4-((4-methyl-2-(3-oxocyclobut-1-en-1-yl) thiazol-5-yl)oxy)phenyl)-2,4-dihydro-3H-1,2,4-triazol-3-one

To a stirred solution of 4-(2,6-difluorobenzyl)-2-(3-fluoro-4-((2-(1-fluoro-3-hydroxycyclobutyl)-4-methylthiazol-5-yl)oxy)phenyl)-2,4-dihydro-3H-1,2,4-triazol-3-one (300 mg, 0.59 mmol) in DCM (10 mL), was added pyridinium chlorochromate (153 mg, 0.71 mmol), and the mixture solution was stirred at rt for 2 hrs. The reaction was quenched with saturated NH₄Cl solution and was diluted with DCM (20 mL), the resulted solution was washed with water (10 mL) and brine (10 mL), dried over anhydrous Na₂SO₄, and after filtration, the filtrate was concentrated under reduced pressure. The residue was purified by silica gel column chromatography, eluted with (PE/EtOAc=2:1) to afford 4-(2,6-difluorobenzyl)-2-(3-fluoro-4-((4-methyl-2-(3-oxocyclobut-1-en-1-yl)thiazol-5-yl)oxy)p henyl)-2,4-dihydro-3H-1,2,4-triazol-3-one (160 mg, 56.0% yield) as a white solid. MS (m/z): 485.1[M+H]⁺.

### Step 2: 4-(2,6-difluorobenzyl)-2-(3-fluoro-4-((2-(3-hydroxy-3-methylcyclobut-1-en-1-yl)-4-methylthiazol-5-yl)oxy)phenyl)-2,4-dihydro-3H-1,2,4-triazol-3-one

To a stirred solution of ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2-oxoacetate (160 mg, 0.33 mmol) in THF (10 mL) at 0° C., under N₂ atmosphere, was added MeMgCl in a THF solution (0.2 mL, 3 M, 0.66 mmol). The reaction was quenched with a saturated NH₄Cl solution and was diluted with EtOAc (20 mL), the resulted solution was washed with water (10 mL) and brine (10 mL), dried over anhydrous Na₂SO₄, and after filtration, the filtrate was concentrated under reduced pressure. The residue was purified by silica gel column chromatography, eluted with (PE/EtOAc) (1:1) to afford 4-(2,6-difluorobenzyl)-2-(3-fluoro-4-((2-(3-hydroxy-3-methylcyclobut-1-en-1-yl)-4-methylthiazol-5-yl)oxy)phenyl)-2,4-dihydro-3H-1,2,4-triazol-3-one (100 mg, 60.6% yield) as a white solid. MS (m/z): 501.2 [M+H]⁺.

### Step 3: 4-(2,6-difluorobenzyl)-2-(3-fluoro-4-((2-(3-hydroxy-3-methylcyclobutyl)-4-methylthiazol-5-yl)oxy)phenyl)-2,4-dihydro-3H-1,2,4-triazol-3-one

To a stirred solution of 4-(2,6-difluorobenzyl)-2-(3-fluoro-4-((2-(3-hydroxy-3-methylcyclobut-1-en-1-yl)-4-methylthiazol-5-yl)oxy)phenyl)-2,4-dihydro-3H-1,2,4-triazol-3-one (100 mg, 0.20 mmol) in MeOH (10 mL) was added Pd/C (10%, 20 mg) under H₂ atmosphere and the reaction solution was stirred at rt for 4 hrs. The resulted solution was filtrated and concentrated under reduced pressure. The residue was purified by TLC. (DCM/MeOH) (30/1) to afford the titled compound product-1 (50 mg, 49.8% yield) as a white solid. MS (m/z): 503.2 [M+H]⁺. ¹H NMR (400 MHz, CDCl₃) δ 7.92 (d, J=12.0 Hz, 1H), 7.76 (d, J=8.4 Hz, 1H), 7.58 (s, 1H), 7.43-7.32 (m, 1H), 7.09-6.96 (m, 3H), 4.97 (s, 2H), 4.06-3.92 (m, 1H), 2.62 (t, J=9.2 Hz, 2H), 2.53-2.45 (m, 2H), 2.39 (s, 3H), 1.44 (s, 3H).

| Com- | | | Appear- | | MS |
|---|---|---|---|---|---|
| pound | | | ance | | (m/z) |
| No. | | | and | | [M + |
| Method | Activity | Structure and Name | Yield | ¹H NMR Data | H]⁺ |
| Exam- ple 119 Method 25 | ++++ | 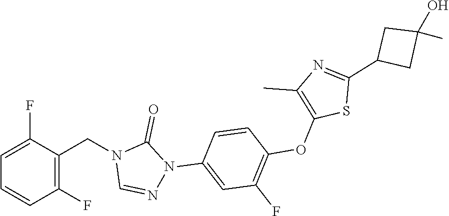 [SMILES: Cc1nc(C2CC(C)(O)C2)sc1Oc1ccc(-n2ncn(Cc3c(F)cccc3F)c2=O)cc1F] | white solid, yield: 49.8% | ¹H NMR (400 MHz, CDCl₃) δ 7.92 (d, J = 12.0 Hz, 1H), 7.76 (d, J = 8.4 Hz, 1H), 7.58 (s, 1H), 7.43-7.32 (m, 1H), 7.09-6.96 (m, 3H), 4.97 (s, 2H), 4.06-3.92 (m, 1H), 2.62 (t, J = 9.2 Hz, 2H), 2.53-2.45 (m, 2H), 2.39 (s, 3H), 1.44 (s, 3H). | 503.2 |
| | | 4-(2,6-difluorobenzyl)-2-(3-fluoro- | | | |
| | | 4-((2-(3-hydroxy-3-methylcyclo- | | | |
| | | butyl)-4-methylthiazol-5-yl) | | | |
| | | oxy)phenyl)-2,4-dihydro-3H- | | | |
| | | 1,2,4-triazol-3-one | | | |

### Example 120: Method 26

### 5-(4-(4-(2,6-difluorobenzyl)-5-oxo-4,5-dihydro-1H-1,2,4-triazol-1-yl)-2-fluorophenoxy)-2-methylthiazole-4-carbonitrile

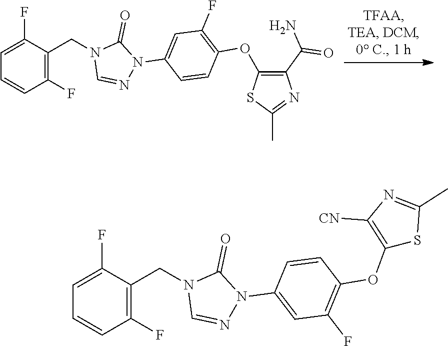

To a stirred solution of 5-(4-(4-(2,6-difluorobenzyl)-5-oxo-4,5-dihydro-1H-1,2,4-triazol-1-yl)-2-fluorophenoxy)-2-methylthiazole-4-carboxamide (139 mg, 0.301 mmol) and TEA (91 mg, 0.904 mmol) in DCM (10 mL) were added TFAA (127 mg, 0.602 mmol). The resulted mixture was stirred for additional 1 h at 0° C. The resulted mixture was diluted with water (10 mL). The resulted mixture was extracted with EtOAc (3×20 mL). The combined organic layers were washed with brine, dried over anhydrous Na₂SO₄, and after filtration, the filtrate was concentrated under reduced pressure. The residue was purified by reverse phase flash chromatography with the following conditions: column: Spherical C₁₈ 40-60 um, 40 g; Mobile phase B: ACN; Flow rate: 40 mL/min; Gradient: 35% B-60% B in 20 min; Detector: 254 nm. The fractions containing the desired product were collected at 51% B and concentrated under reduced pressure to afford the titled product (9.1 mg, 60 yield) as a white solid. LC-MS (m/z): 444.0 [M+H]⁺. ¹H NMR (400 MHz, DMSO) δ 8.39 (s, 1H), 7.96 (dd, J=12.5, 2.4 Hz, 1H), 7.80 (d, J=9.1 Hz, 1H), 7170 (t, JH 8.9 Hz, 1)H), 7.55-7.43 (t, H), 7.16 (t, J=8.1 Hz, 2H), 4.99 (s, 2H), 2.58 (s, 3H).

Examples (Compounds) 121-125 were synthesized using a similar method to that used in Example 120.

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Method 26 Exam- ple 120 | ++++ | 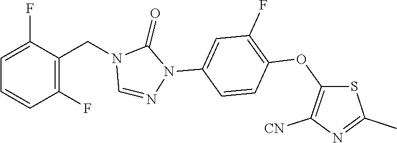 | white solid, yield: 6% | ¹H NMR (400 MHz, DMSO-d₆) δ 8.39 (s, 1H), 7.96 (dd, J = 12.5, 2.4 Hz, 1H), 7.80 (d, J = 9.1 Hz, 1H), 7.70 (t, J = 8.9 Hz, 1H), 7.55- 7.43 (m, 1H), 7.16 (t, J = 8.1 Hz, 2H), 4.99 (s, 2H), 2.58 (s, 3H). | 444.0 |
| | | 5-(4-(4-(2,6-difluorobenzyl)-5- | | | |
| | | oxo-4,5-dihydro-1H-1,2,4- | | | |
| | | triazol-1-yl)-2-fluorophenoxy)-2- | | | |
| | | methylthiazole-4-carbonitrile | | | |
| Exam- ple 121 Method 26 | +++ | 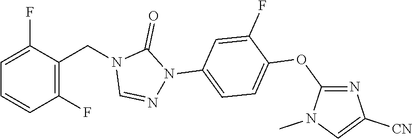 [SMILES: Cn1cc(C#N)nc1Oc1ccc(-n2ncn(Cc3c(F)cccc3F)c2=O)cc1F] | white solid, yield: 65.1% | ¹H NMR (400 MHz, DMSO-d₆) δ 8.37 (s, 1H), 7.95 (s, 1H), 7.90 (dd, J = 12.4, 2.4 Hz, 1H), 7.77 (d, J = 9.1 Hz, 1H), 7.61 (t, J = 8.9 Hz, 1H), 7.53-7.44 (m, 1H), 7.16 (t, J = 8.1 Hz, 2H), 4.99 (s, 2H), 3.64 (s, 3H). | 427.0 |
| | | 2-(4-(4-(2,6-difluorobenzyl)-5- | | | |
| | | oxo-4,5-dihydro-1H-1,2,4-triazol- | | | |
| | | 1-yl)-2-fluorophenoxy)-1-methyl- | | | |
| | | 1H-imidazole-4-carbonitrile | | | |
| Exam- ple 122 Method 26 | ++++ | 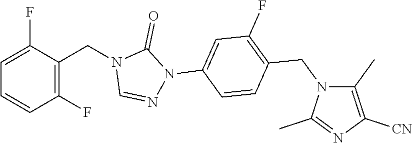 [SMILES: Cc1nc(C#N)c(C)n1Cc1ccc(-n2ncn(Cc3c(F)cccc3F)c2=O)cc1F] | white solid, yield: 9.0% | ¹H NMR (400 MHz, CDCl₃) δ 7.89 (m, 1H), 7.81 (m, 1H), 7.58 (s, 1H), 7.40-7.34 (m, 1H), 7.09 (m, 1H), 6.98 (m, 2H), 5.15 (s, 2H), 4.97 (s, 2H), 2.39 (s, 3H), 2.35 (s, 3H). | 439.1 |
| | | 1-(4-(4-(2,6-difluorobenzyl)-5- | | | |
| | | oxo-4,5-dihydro-1H-1,2,4-triazol- | | | |
| | | 1-yl)-2-fluorobenzyl)-2,5-dimethyl- | | | |
| | | 1H-imidazole-4-carbonitrile | | | |
| Exam- ple 123 Method 26 | +++ | 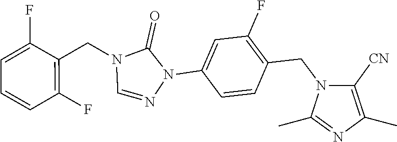 [SMILES: Cc1nc(C)n(Cc2ccc(-n3ncn(Cc4c(F)cccc4F)c3=O)cc2F)c1C#N] | white solid, yield: 1.9% | ¹H NMR (400 MHz, CDCl₃) δ 7.91 (m, 1H), 7.76 (m, 1H), 7.59 (s, 1H), 7.41-7.34 (m, 1H), 6.99 (m, 2H), 6.69 (m, 1H), 5.06 (s, 2H), 4.97 (s, 2H), 2.35 (s, 3H), 2.29 (s, 3H). | 439.1 |
| | | 1-(4-(4-(2,6-difluorobenzyl)-5- | | | |
| | | oxo-4,5-dihydro-1H-1,2,4-triazol- | | | |
| | | 1-yl)-2-fluorobenzyl)-2,4-dimethyl- | | | |
| | | 1H-imidazole-5-carbonitrile | | | |
| Exam- ple 124 Method 26 | ++++ | 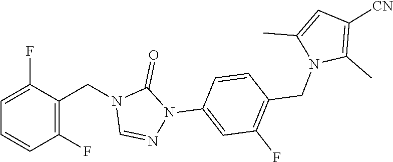 [SMILES: Cc1cc(C#N)c(C)n1Cc1ccc(-n2ncn(Cc3c(F)cccc3F)c2=O)cc1F] | white solid, yield: 9% | ¹H NMR (400 MHz, CD₃OD_SPE) δ 8.01 (s, 1H), 7.79 (dd, J = 12.0, 2.0 Hz, 1H), 7.70 (d, J = 8.5 Hz, 1H), 7.49-7.40 (m, 1H), 7.05 (t, J = 8.1 Hz, 2H), 6.59 (t, J = 8.4 Hz, 1H), 6.13 (s, 1H), 5.19 (s, 2H), 5.03 (s, 2H), 2.29 (s, 3H), 2.13 (s, 3H). | 438.1 |
| | | 1-(4-(4-(2,6-difluorobenzyl)-5- | | | |
| | | oxo-4,5-dihydro-1H-1,2,4-triazol- | | | |
| | | 1-yl)-2-fluorobenzyl)-2,5-dimethyl- | | | |
| | | 1H-pyrrole-3-carbonitrile | | | |
| Exam- ple 125 Method 26 | ++++ | 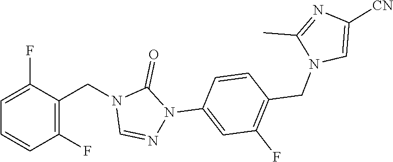 [SMILES: Cc1nc(C#N)cn1Cc1ccc(-n2ncn(Cc3c(F)cccc3F)c2=O)cc1F] | white solid, yield: 69.5% | ¹H NMR (400 MHz, CDCl₃) δ 7.95-7.88 (m, 2H), 7.61 (s, 1H), 7.44 (s, 1H), 7.37 (m, 1H), 7.18 (s, 1H), 7.00 (d, J = 7.7 Hz, 2H), 5.14 (s, 2H), 4.98 (s, 2H), 2.59 (s, 3H). | 425.1 |
| | | 1-(4-(4-(2,6-difluorobenzyl)-5- | | | |
| | | oxo-4,5-dihydro-1H-1,2,4-triazol- | | | |
| | | 1-yl)-2-fluorobenzyl)-2-methyl- | | | |
| | | 1H-imidazole-4-carbonitrile | | | |

### Example 126: Method 27

### 4-(2,6-difluorobenzyl)-2-(3-fluoro-4-((4-(2-hydroxypropan-2-yl)-2-methylthiazol-5-yl)oxy)phenyl)-2,4-dihydro-3H-1,2,4-triazol-3-one

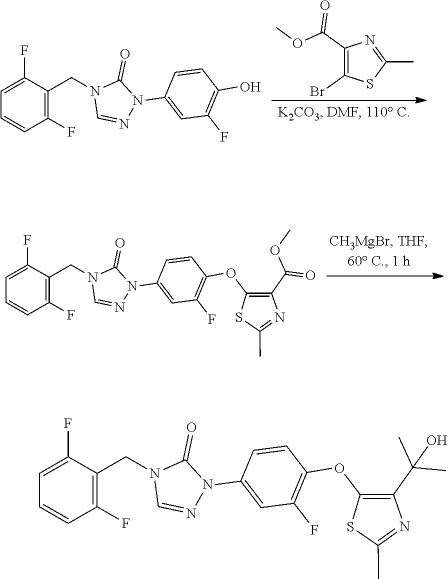

### Step 1: methyl 5-(4-(4-(2,6-difluorobenzyl)-5-oxo-4,5-dihydro-1H-1,2,4-triazol-1-yl)-2-fluorophenoxy)-2-methylthiazole-4-carboxylate

To a stirred solution of 2,4-dichloro-5-fluoropyrimidine (300 mg, 0.934 mmol) and methyl 5-bromo-2-methylthiazole-4-carboxylate (220 mg, 0.934 mmol) in DMF (15 mL) was added K₂CO₃ (194 mg, 1.40 mmol). The resulted mixture was stirred for additional 16 h at 110° C. The mixture was allowed to cool down to rt. The resulted mixture was diluted with water (30 mL). The resulted mixture was extracted with EtOAc (3×50 mL). The combined organic layers were washed with brine, dried over anhydrous Na₂SO₄, and after filtration, the filtrate was concentrated under reduced pressure. The residue was purified by silica gel column chromatography, eluted with (PE:EtOAc) (1:1) to afford methyl 5-(4-(4-(2,6-difluorobenzyl)-5-oxo-4,5-dihydro-1H-1,2,4-triazol-1-yl)-2-fluorophenoxy)-2-met hylthiazole-4-carboxylate(210 mg, 42.5% yield) as a yellow solid. MS (m/z): 477.0 [M+H]⁺.

### Step 2: 4-(2,6-difluorobenzyl)-2-(3-fluoro-4-((4-(2-hydroxypropan-2-yl)-2-methylthiazol-5-yl)oxy)phenyl)-2,4-dihydro-3H-1,2,4-triazol-3-one

To a stirred solution of methyl 5-(4-(4-(2,6-difluorobenzyl)-5-oxo-4,5-dihydro-1H-1,2,4-triazol-1-yl)-2-fluorophenoxy)-2-methylthiazole-4-carboxylate(60 mg, 0.126 mmol) in THF (10 mL) were added CH3MgBr (1.26 mL, 1.26 mmol). The resulted mixture was stirred for additional 1 h at 60° C. The resulted mixture was diluted with water (20 mL). The resulted mixture was extracted with EtOAc (3×30 mL). The combined organic layers were washed with brine, dried over anhydrous Na₂SO₄, and after filtration, the filtrate was concentrated under reduced pressure. The residue was purified by reverse phase flash chromatography with the following conditions: column: Spherical C18 40-60 um, 40 g; Mobile phase B: ACN; Flow rate: 40 mL/min; Gradient: 35% B-60% B in 20 min; Detector: 254 nm. The fractions containing the desired product were collected at 51% B and concentrated under reduced pressure to afford the titled compound 4-(2,6-difluorobenzyl)-2-(3-fluoro-4-((4-(2-hydroxypropan-2-yl)-2-methylthiazol-5-yl)oxy)phenyl)-2,4-dihydro-3H-1,2,4-triazol-3-one (9.3 mg, 15% yield) as a white solid. LC-MS (m/z): 477.0 [M+H]⁺. ¹H NMR (400 MHz, CD₃OD) δ 8.00 (s, 1H), 7.84 (dd, J=12.4, 2.4 Hz, 1H), 7.70 (dd, J=9.1, 1.6 Hz, 1H), 7.51-7.39 (m, 1H), 7.19 (t, J=9.0 Hz, 1H), 7.06 (t, J=8.1 Hz, 2H), 5.03 (s, 2H), 2.57 (s, 3H), 1.57 (s, 6H).

Examples (Compounds) 127-137 were synthesized using a similar method to that used in Example 126.

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Exam- ple 126 Method 27 | +++ | 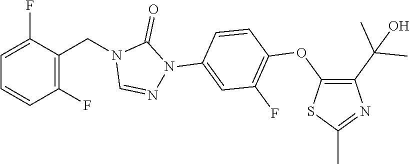 [SMILES: Cc1nc(C(C)(C)O)c(Oc2ccc(-n3ncn(Cc4c(F)cccc4F)c3=O)cc2F)s1] | yellow solid, yield: 15% | ¹H NMR (400 MHz, CD₃OD) δ 8.00 (s, 1H), 7.84 (dd, J = 12.4, 2.4 Hz, 1H), 7.70 (dd, J = 9.1, 1.6 Hz, 1H), 7.51-7.39 (m, 1H), 7.19 (t, J = 9.0 Hz, 1H), 7.06 (t, J = 8.1 Hz, 2H), 5.03 (s, 2H), 2.57 (s, 3H), 1.57 (s, 6H). | 477.0 |
| | | 4-(2,6-difluorobenzyl)-2-(3-fluoro- | | | |
| | | 4-((4-(2-hydroxypropan-2-yl)- | | | |
| | | 2-methylthiazol-5-yl)oxy)phenyl)- | | | |
| | | 2,4-dihydro-3H-1,2,4-triazol- | | | |
| | | 3-one | | | |
| Exam- ple 127 Method 27 | ++++ | 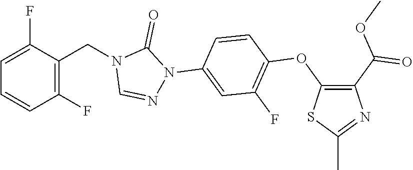 [SMILES: COC(=O)c1nc(C)sc1Oc1ccc(-n2ncn(Cc3c(F)cccc3F)c2=O)cc1F] | yellow solid, yield: 42.5% | NA | 477.0 |
| | | methyl | | | |
| | | 5-(4-(4-(2,6-difluorobenzyl)-5- | | | |
| | | oxo-4,5-dihydro-1H-1,2,4-triazol- | | | |
| | | 1-yl)-2-fluorophenoxy)-2-methyl- | | | |
| | | thiazole-4-carboxylate | | | |
| Exam- ple 128 Method 27 | +++ | 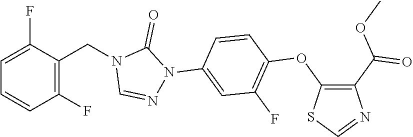 [SMILES: COC(=O)c1ncsc1Oc1ccc(-n2ncn(Cc3c(F)cccc3F)c2=O)cc1F] | yellow solid, yield: 42.6% | ¹H NMR (400 MHz, CD₃OD_SPE) δ 8.53 (s, 1H), 8.03 (s, 1H), 7.96 (dd, J = 12.3, 2.5 Hz, 1H), 7.85 7.76 (m, 1H), 7.56-7.35 (m, 2H), 7.06 (t, J = 8.1 Hz, 2H), 5.04 (s, 2H), 3.88 (s, 3H). | 463.0 |
| | | methyl | | | |
| | | 5-(4-(4-(2,6-difluorobenzyl)-5- | | | |
| | | oxo-4,5-dihydro-1H-1,2,4-triazol- | | | |
| | | 1-yl)-2-fluorophenoxy)thiazole- | | | |
| | | 4-carboxylate | | | |
| Exam- ple 129 Method 27 | +++ | 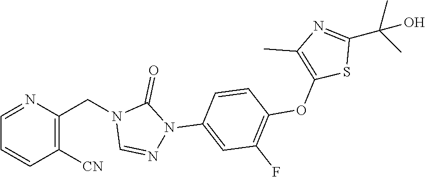 [SMILES: Cc1nc(C(C)(C)O)sc1Oc1ccc(-n2ncn(Cc3ncccc3C#N)c2=O)cc1F] | off-white solid, yield: 11.6% | ¹H NMR (400 MHz, CDCl₃) δ 8.76 (d, J = 2.4 Hz, 1H), 8.05- 7.98 (m, 1H), 7.95-7.84 (m, 1H), 7.75 (d, J = 5.2 Hz, 2H), 7.39- 7.44 (m, 1H), 7.03 (t, J = 8.8 Hz, 1H), 5.25 (s, 2H), 2.29 (s, 3H), 1.64 (s, 6H). | 467.5 |
| | | 2-((1-(3-fluoro-4-((2-(2-hydroxy- | | | |
| | | propan-2-yl)-4-methylthiazol-5- | | | |
| | | yl)oxy)phenyl)-5-oxo-1,5-dihydro- | | | |
| | | 4H-1,2,4-triazol-4-yl) | | | |
| | | methyl)nicotinonitrile | | | |
| Exam- ple 130 Method 27 | ++++ | 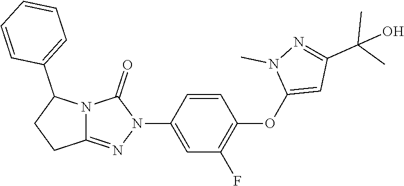 [SMILES: Cn1nc(C(C)(C)O)cc1Oc1ccc(-n2nc3n(c2=O)C(c2ccccc2)CC3)cc1F] | off-white solid, yield: 10.6% | ¹H NMR (400 MHz, CDCl₃) δ 7.94 (d, J = 12.4 Hz, 1H), 7.80 (d, J = 8.8 Hz, 1H), 7.343-7.31 (m, 3H), 7.23 (d, J = 8.2 Hz, 4H), 5.44 (s, 1H), 5.32-5.26 (m, 1H), 3.90 (d, J = 13.1 Hz, 3H), 3.16- 2.87 (m, 4H), 1.55 (s, 6H). | 450.4 |
| | | 2-(3-fluoro-4-((3-(2-hydroxypro- | | | |
| | | pan-2-yl)-1-methyl-1H-pyrazol- | | | |
| | | 5-yl)oxy)phenyl)-5-phenyl-2,5,6,7- | | | |
| | | tetrahydro-3H-pyrrolo[2,1-c] | | | |
| | | [1,2,4]triazol-3-one | | | |
| Exam- ple 131 Method 27 | + | 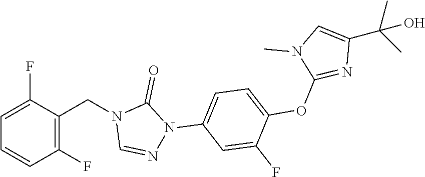 [SMILES: Cn1cc(C(C)(C)O)nc1Oc1ccc(-n2ncn(Cc3c(F)cccc3F)c2=O)cc1F] | off-white solid, yield: 30.0% | ¹H NMR (400 MHz, MeOD) δ 8.47 (s, 1H), 8.01 (s, 1H), 7.88 (dd, J = 12.3, 2.4 Hz, 1H), 7.76 (d, J = 9.1 Hz, 1H), 7.49-7.39 (m, 1H), 7.33 (t, J = 8.9 Hz, 1H), 7.06 (t, J = 8.1 Hz, 2H), 6.70 (s, 1H), 5.03 (s, 2H), 3.54 (s, 3H), 1.43 (s, 6H). | 460.1 |
| | | 4-(2,6-difluorobenzyl)-2-(3-fluoro- | | | |
| | | 4-((4-(2-hydroxypropan-2-yl)- | | | |
| | | 1-methyl-1H-imidazol-2-yl)oxy) | | | |
| | | phenyl)-2,4-dihydro-3H-1,2,4- | | | |
| | | triazol-3-one | | | |
| Exam- ple 132 Method 27 | ++ | 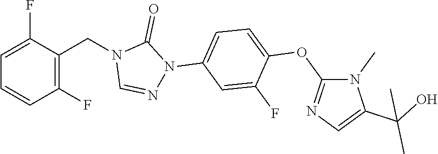 [SMILES: Cn1c(C(C)(C)O)cnc1Oc1ccc(-n2ncn(Cc3c(F)cccc3F)c2=O)cc1F] | white solid, yield: 26.2% | ¹H NMR (400 MHz, CDCl₃) δ 7.97 (s, 1H), 7.84 (dd, J = 11.1, 6.5 Hz, 1H), 7.57 (s, 1H), 7.35 (m, 1H), 7.25 (s, 1H), 6.98 (dd, J = 9.2, 6.6 Hz, 3H), 4.95 (s, 2H), 3.83 (s, 3H), 1.63 (s, 6H). | 460.1 |
| | | 4-(2,6-difluorobenzyl)-2-(3-fluoro- | | | |
| | | 4-((5-(2-hydroxypropan-2-yl)- | | | |
| | | 1-methyl-1H-imidazol-2-yl)oxy) | | | |
| | | phenyl)-2,4-dihydro-3H-1,2,4- | | | |
| | | triazol-3-one | | | |
| Exam- ple 133 Method 27 | +++ | 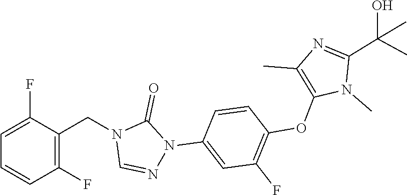 [SMILES: Cc1nc(C(C)(C)O)n(C)c1Oc1ccc(-n2ncn(Cc3c(F)cccc3F)c2=O)cc1F] | white solid, yield: 4.2% | ¹H NMR (400 MHz, CDCl₃) δ 7.96 (dd, J = 12.3, 2.3 Hz, 1H), 7.71 (d, J = 9.1 Hz, 1H), 7.58 (s, 1H), 7.37 (t, J = 6.7 Hz, 1H), 6.99 (t, J = 8.0 Hz, 3H), 4.97 (s, 2H), 3.73 (s, 3H), 2.14 (s, 3H), 1.72 (s, 6H). | 474.1 |
| | | 4-(2,6-difluorobenzyl)-2-(3-fluoro- | | | |
| | | 4-((2-(2-hydroxypropan-2-yl)- | | | |
| | | 1,4-dimethyl-1H-imidazol-5-yl) | | | |
| | | oxy)phenyl)-2,4-dihydro-3H- | | | |
| | | 1,2,4-triazol-3-one | | | |
| Exam- ple 134 Method 27 | ++++ | 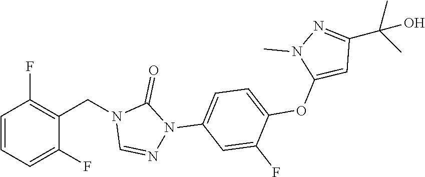 [SMILES: Cn1nc(C(C)(C)O)cc1Oc1ccc(-n2ncn(Cc3c(F)cccc3F)c2=O)cc1F] | white solid, yield: 45.6% | ¹H NMR (400 MHz, CDCl₃) δ 7.99-7.89 (m, 1H), 7.87-7.77 (m, 1H), 7.58 (d, J = 9.5 Hz, 1H), 7.42-7.34 (m, 1H), 7.26-7.21 (m, 1H), 7.03-6.96 (m, 2H), 6.06 (d, J = 6.1 Hz, 1H), 5.34 (s, 1H), 4.97 (d, J = 7.4 Hz, 2H), 3.83 (s, 3H), 1.56 (s, 3H), 1.26 (s, 3H). | 460.4 |
| | | 4-(2,6-difluorobenzyl)-2-(3-fluoro- | | | |
| | | 4-((3-(2-hydroxypropan-2-yl)- | | | |
| | | 1-methyl-1H-pyrazol-5-yl)oxy) | | | |
| | | phenyl)-2,4-dihydro-3H-1,2,4- | | | |
| | | triazol-3-one | | | |
| Exam- ple 135 Method 27 | ++ | 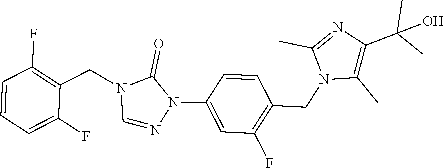 [SMILES: Cc1nc(C(C)(C)O)c(C)n1Cc1ccc(-n2ncn(Cc3c(F)cccc3F)c2=O)cc1F] | white solid, yield: 4.5% | ¹H NMR (400 MHz, CDCl₃) δ 7.90 (m, 1H), 7.75 (m, 1H), 7.59 (s, 1H), 7.37 (m, 1H), 6.98 (m, 3H), 6.69 (m, 1H), 5.06 (s, 2H), 4.97 (s, 2H), 2.47 (s, 3H), 2.21 (s, 3H), 1.62 (s, 6H). | 472.1 |
| | | 4-(2,6-difluorobenzyl)-2-(3-fluoro- | | | |
| | | 4-((4-(2-hydroxypropan-2-yl)- | | | |
| | | 2,5-dimethyl-1H-imidazol-1-yl) | | | |
| | | methyl)phenyl)-2,4-dihydro-3H- | | | |
| | | 1,2,4-triazol-3-one | | | |
| Exam- ple 136 Method 27 | ++ | 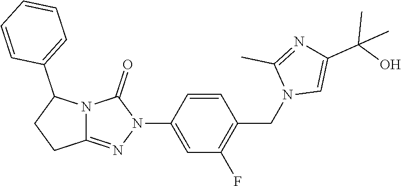 [SMILES: Cc1nc(C(C)(C)O)cn1Cc1ccc(-n2nc3n(c2=O)C(c2ccccc2)CC3)cc1F] | white solid, yield: 9.4% | ¹H NMR (400 MHz, CDCl₃) δ 7.83 (m, 2H), 7.41-7.35 (m, 3H), 7.24 (s, 2H), 7.09 (m, 1H), 6.67 (s, 1H), 5.30-5.26 (m, 1H), 5.02 (s, 2H), 3.12-2.88 (m, 4H), 2.51 (s, 3H), 1.57 (s, 6H). | 448.2 |
| | | 2-(3-fluoro-4-((4-(2-hydroxypro- | | | |
| | | pan-2-yl)-2-methyl-1H-imidazol- | | | |
| | | 1-yl)methyl)phenyl)-5-phenyl- | | | |
| | | 2,5,6,7-tetrahydro-3H-pyrrolo | | | |
| | | [2,1-c][1,2,4]triazol-3-one | | | |
| Exam- ple 137 Method 27 | ++ | 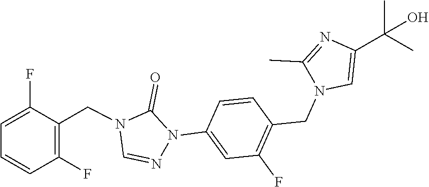 [SMILES: Cc1nc(C(C)(C)O)cn1Cc1ccc(-n2ncn(Cc3c(F)cccc3F)c2=O)cc1F] | white solid, yield: 10.2% | 1H NMR (400 MHz, CDCl3) δ 7.94-7.86 (m, 2H), 7.61 (s, 1H), 7.37 (m, 1H), 7.18 (m, 1H), 6.99 (m, 2H), 6.67 (s, 1H), 5.06 (s, 2H), 4.98 (s, 2H), 2.61 (s, 3H), 1.59 (s, 6H). | 458.2 |
| | | 4-(2,6-difluorobenzyl)-2-(3-fluoro- | | | |
| | | 4-((4-(2-hydroxypropan-2-yl)- | | | |
| | | 2-methyl-1H-imidazol-1-yl)methyl) | | | |
| | | phenyl)-2,4-dihydro-3H- | | | |
| | | 1,2,4-triazol-3-one | | | |

### Example 138: Method 28

### 2-(5-(4-(4-(2,6-difluorobenzyl)-5-oxo-4,5-dihydro-1H-1,2,4-triazol-1-yl)-2-fluorophenoxy)-2-methylthiazol-4-yl)acetamide

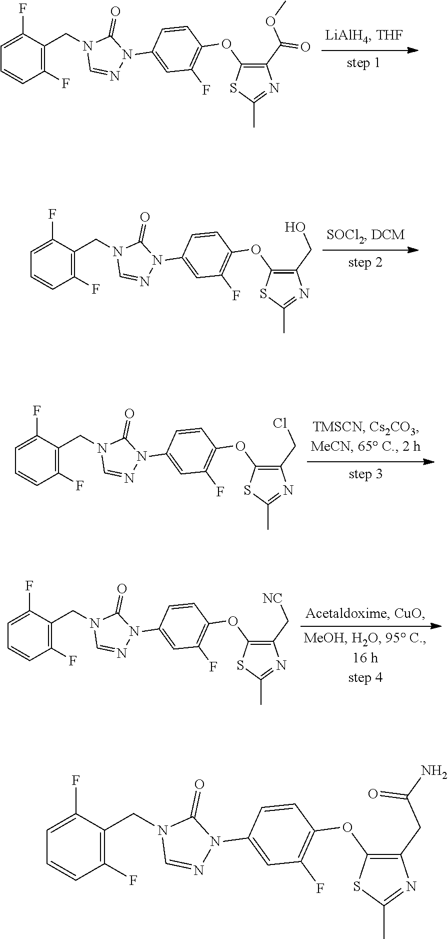 [SMILES: COC(=O)c1nc(C)sc1Oc1ccc(-n2ncn(Cc3c(F)cccc3F)c2=O)cc1F ; Cc1nc(CO)c(Oc2ccc(-n3ncn(Cc4c(F)cccc4F)c3=O)cc2F)s1 ; Cc1nc(CCl)c(Oc2ccc(-n3ncn(Cc4c(F)cccc4F)c3=O)cc2F)s1 ; Cc1nc(CC#N)c(Oc2ccc(-n3ncn(Cc4c(F)cccc4F)c3=O)cc2F)s1 ; Cc1nc(CC(N)=O)c(Oc2ccc(-n3ncn(Cc4c(F)cccc4F)c3=O)cc2F)s1]

### Step 1: 4-(2,6-difluorobenzyl)-2-(3-fluoro-4-((4-(hydroxymethyl)-2-methylthiazol-5-yl)oxy)phenyl)-2,4-dihydro-3H-1,2,4-triazol-3-one

To a stirred solution of methyl 5-(4-(4-(2,6-difluorobenzyl)-5-oxo-4,5-dihydro-1H-1,2,4-triazol-1-yl)-2-fluorophenoxy)-2-methylthiazole-4-carboxylate (323 mg, 0.678 mmol) in THF (10 mL) were added LiAlH₄ (51.5 mg, 1.36 mmol). The resulted mixture was stirred for additional 2 h at 25° C. The resulted mixture was quenched with water (0.5 mL), 10% NaOH solution (1.5 mL) and water (0.5 mL). The resulted mixture was filtered with celite and extracted with EtOAc (3×30 mL). The combined organic layers were washed with brine, dried over anhydrous Na₂SO₄, and after filtration, the filtrate was concentrated under reduced pressure. The residue was purified by silica gel column chromatography, eluted with (PE:EtOAc) (1:1) to afford 4-(2,6-difluorobenzyl)-2-(3-fluoro-4-((4-(hydroxymethyl)-2-methylthiazol-5-yl)oxy)phenyl)-2,4-dihydro-3H-1,2,4-triazol-3-one (167 mg, 49.4% yield) as a yellow solid. MS (m/z): 449.1 [M+H]⁺.

### Step 2: 2-(4-((4-(chloromethyl)-2-methylthiazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(3-fluoro-4-((4-(hydroxymethyl)-2-methylthiazol-5-yl)oxy)phenyl)-2,4-dihydro-3H-1,2,4-triazol-3-one (167 mg, 0.372 mmol) in DCM (10 mL) was added SOCl₂ (133 mg, 1.12 mmol). The resulted mixture was stirred for additional 1 h at 25° C. The resulted mixture was adjusted to pH 8 with NaHCO₃ and extracted with EtOAc (3×30 mL). The combined organic layers were washed with brine, dried over anhydrous Na₂SO₄, and after filtration, the filtrate was concentrated under reduced pressure. The residue was purified by silica gel column chromatography, eluted with (PE:EtOAc) (10:1) to afford 2-(4-((4-(chloromethyl)-2-methylthiazol-5-yl)oxy)-3-fluorophenyl)-4-(2,6-difluorobenzyl)-2,4-dihydro-3H-1,2,4-triazol-3-one (132 g, 68% yield) as a yellow oil. MS (m/z): 467.0 [M+H]⁺.

### Step 3: 2-(5-(4-(4-(2,6-difluorobenzyl)-5-oxo-4,5-dihydro-1H-1,2,4-triazol-1-yl)-2-fluorophenoxy)-2-methylthiazol-4-yl)acetonitrile

To a stirred solution of 2-(4-((4-(chloromethyl)-2-methylthiazol-5-yl)oxy)-3-fluorophenyl)-4-(2,6-difluorobenzyl)-2,4-dihydro-3H-1,2,4-triazol-3-one (132 mg, 0.283 mmol), were added TMSCN (42 mg, 0.424 mmol) and Cs₂CO₃ (184 mg, 0.565 mmol) in MeCN (10 mL). The resulted mixture was stirred for additional 2 h at 65° C. The resulted mixture was diluted with water (10 mL). The resulted mixture was extracted with EtOAc (3×20 mL). The combined organic layers were washed with brine, dried over anhydrous Na₂SO₄, and after filtration, the filtrate was concentrated under reduced pressure. The residue was purified by silica gel column chromatography, eluted with (PE:EtOAc) (2:1) to afford 2-(5-(4-(4-(2,6-difluorobenzyl)-5-oxo-4,5-dihydro-1H-1,2,4-triazol-1-yl)-2-fluorophenoxy)-2-methylthiazol-4-yl)acetonitrile (89 mg, 62% yield) as a yellow solid. LC-MS (m/z): 458.1[M+H]⁺.

### Step 4: 2-(5-(4-(4-(2,6-difluorobenzyl)-5-oxo-4,5-dihydro-1H-1,2,4-triazol-1-yl)-2-fluorophenoxy)-2-methylthiazol-4-yl)acetamide

To a stirred solution of 2-(5-(4-(4-(2,6-difluorobenzyl)-5-oxo-4,5-dihydro-1H-1,2,4-triazol-1-yl)-2-fluorophenoxy)-2-methylthiazol-4-yl)acetonitrile (89 mg, 0.195 mmol), were added CuO (30.7 mg, 0.389 mmol) and Acetaldoxime (34.4 mg, 0.584 mmol) in MeOH (5 mL) and H₂O (5 mL). The resulted mixture was stirred for additional 16 h at 95° C. The resulted mixture was diluted with water (10 mL). The resulted mixture was extracted with EtOAc (3×20 mL). The combined organic layers were washed with brine, dried over anhydrous Na₂SO₄, and after filtration, the filtrate was concentrated under reduced pressure. The residue was purified by reverse phase flash chromatography with the following conditions: column: Spherical Cis 40-60 um, 40 g; Mobile phase B: ACN; Flow rate: 40 mL/min; Gradient: 35% B-60% B in 20 min; Detector: 254 nm. The fractions containing the desired product were collected at 51% B and concentrated under reduced pressure to afford the titled compound 2-(5-(4-(4-(2,6-difluorobenzyl)-5-oxo-4,5-dihydro-1H-1,2,4-triazol-1-yl)-2-fluorophenoxy)-2-methylthiazol-4-yl)acetamide (24.8 mg, 25% yield) as a white solid. LC-MS (m/z): 476.0 [M+H]⁺. ¹H NMR (400 MHz, DMSO) δ 8.34 (s, 1H), 7.85 (dd, J=12.6, 2.4 Hz, 1H), 7.64 (d, J=9.2 Hz, 1H), 7.53-7.44 (m, 1H), 7.35 (dd, J 17.8, 8.7 Hz, 2H), 7.16 (t, J 8.1 Hz, 2H), 6.94 (s, 1H), 4.97 (s, 2H), 3.40 (s, 2H), 2.55 (s, 3H).

Examples (Compounds) 139-143 were synthesized using a similar method to that used in Example 138.

| | | | Ap- | | |
|---|---|---|---|---|---|
| Com- | | | pear- | | MS |
| pound | | | ance | | (m/z) |
| No. | | | and | | [M + |
| Method | Activity | Structure and Name | Yield | ¹H NMR Data | H]⁺ |
| Exam- ple 138 Method 28 | ++++ | 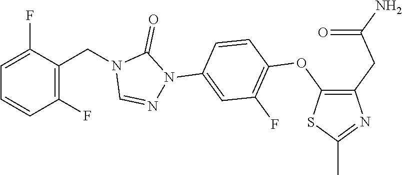 [SMILES: Cc1nc(CC(N)=O)c(Oc2ccc(-n3ncn(Cc4c(F)cccc4F)c3=O)cc2F)s1] | yellow solid, yield: 25% | ¹H NMR (400 MHz, DMSO-d₆) δ 8.34 (s, 1H), 7.85 (dd, J = 12.6, 2.4 Hz, 1H), 7.64 (d, J = 9.2 Hz, 1H), 7.53-7.44 (m, 1H), 7.35 (dd, J = 17.8, 8.7 Hz, 2H), 7.16 (t, J = 8.1 Hz, 2H), 6.94 (s, 1H), 4.97 (s, 2H), 3.40 (s, 2H), 2.55 (s, 3H). | 476.0 |
| | | 2-(5-(4-(4-(2,6-difluorobenzyl)- | | | |
| | | 5-oxo-4,5-dihydro-1H-1,2,4- | | | |
| | | triazol-1-yl)-2-fluorophenoxy)-2- | | | |
| | | methylthiazol-4-yl)acetamide | | | |
| Exam- ple 139 Method 28 | ++++ | 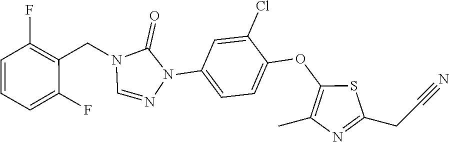 [SMILES: Cc1nc(CC#N)sc1Oc1ccc(-n2ncn(Cc3c(F)cccc3F)c2=O)cc1Cl] | off- white solid, yield: 20.3% | ¹H NMR (400 MHz, CDCl₃) δ 8.15 (d, J = 2.5 Hz, 1H), 7.86-7.84 (m, 1H), 7.58 (s, 1H), 7.37-7.34 (m, 1H), 7.03-6.95 (m, 3H), 4.97 (s, 2H), 3.99 (s, 2H), 2.29 (s, 3H). | 474.2 |
| | | 2-(5-(2-chloro-4-(4-(2,6-difluoro- | | | |
| | | benzyl)-5-oxo-4,5-dihydro-1H- | | | |
| | | 1,2,4-triazol-1-yl)phenoxy)-4- | | | |
| | | methylthiazol-2-yl)acetonitrile | | | |
| Exam- ple 140 Method 28 | ++++ | 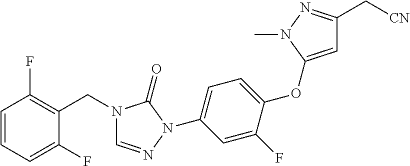 [SMILES: Cn1nc(CC#N)cc1Oc1ccc(-n2ncn(Cc3c(F)cccc3F)c2=O)cc1F] | white solid, yield: 28.0% | ¹H NMR (400 MHz, CDCl₃) δ 7.94 (dd, J = 12.1, 2.5 Hz, 1H), 7.80 (dt, J = 9.0, 2.4, 1.6 Hz, 1H), 7.59 (s, 1H), 7.36 (m, J = 13.1, 7.5, 4.2 Hz, 1H), 7.21 (t, J = 8.8 Hz, 1H), 7.01-6.95 (m, 2H), 5.50 (s, 1H), 4.97 (s, 2H), 3.75 (s, 3H), 3.61 (s, 2H). | 441.4 |
| | | 2-(5-(4-(4-(2,6-difluorobenzyl)- | | | |
| | | 5-oxo-4,5-dihydro-1H-1,2,4- | | | |
| | | triazol-1-yl)-2-fluorophenoxy)-1- | | | |
| | | methyl-1H-pyrazol-3-yl) | | | |
| | | acetonitrile | | | |
| Exam- ple 141 Method 28 | +++ | 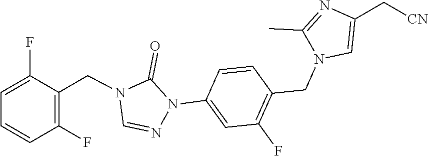 [SMILES: Cc1nc(CC#N)cn1Cc1ccc(-n2ncn(Cc3c(F)cccc3F)c2=O)cc1F] | white solid, yield: 0.63% | ¹H NMR (400 MHz, CDCl₃) δ 7.97 (m, 3H), 7.62 (s, 1H), 7.38 (s, 1H), 6.99 (m, 3H), 5.35 (s, 2H), 4.98 (s, 2H), 4.01 (s, 2H), 2.77 (s, 3H). | 439.1 |
| | | 2-(1-(4-(4-(2,6-difluorobenzyl)- | | | |
| | | 5-oxo-4,5-dihydro-1H-1,2,4- | | | |
| | | triazol-1-yl)-2-fluorobenzyl)-2- | | | |
| | | methyl-1H-imidazol-4-yl) | | | |
| | | acetonitrile | | | |
| Exam- ple 142 Method 28 | ++ | 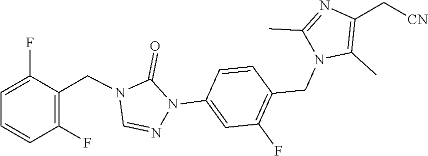 [SMILES: Cc1nc(CC#N)c(C)n1Cc1ccc(-n2ncn(Cc3c(F)cccc3F)c2=O)cc1F] | white solid, yield: 6.9% | ¹H NMR (400 MHz, CDCl₃) δ 7.91 (m, 1H), 7.61 (s, 1H), 7.38 (m, 1H), 7.19 (m, 1H), 6.99 (m, 3H), 5.35 (s, 2H), 4.97 (s, 2H), 3.65 (s, 2H), 2.79 (s, 3H), 2.42 (s, 3H). | 453.1 |
| | | 2-(1-(4-(4-(2,6-difluorobenzyl)- | | | |
| | | 5-oxo-4,5-dihydro-1H-1,2,4- | | | |
| | | triazol-1-yl)-2-fluorobenzyl)-2,5- | | | |
| | | dimethyl-1H-imidazol-4-yl) | | | |
| | | acetonitrile | | | |
| Exam- ple 143 Method 28 | ++++ | 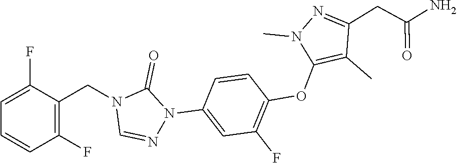 [SMILES: Cc1c(CC(N)=O)nn(C)c1Oc1ccc(-n2ncn(Cc3c(F)cccc3F)c2=O)cc1F] | white solid, yield: 43.4% | ¹H NMR (400 MHz, DMSO-d₆) δ 8.33 (s, 1H), 7.88 (dd, J = 12.4, 2.4 Hz, 1H), 7.63 (dt, J = 9.2, 2.0 Hz, 1H), 7.48 (ddd, J = 15.2, 8.4, 6.4 Hz, 1H), 7.28 (brs, 1H), 7.16 (t, J = 8.0 Hz, 2H), 6.99 (t, J = 9.2 Hz, 1H), 6.88 (brs, 1H), 4.97 (s, 2H), 3.56 (s, 3H), 3.29 (s, 2H), 1.67 (s, 3H). | 473.2 |
| | | 2-(5-(4-(4-(2,6-difluorobenzyl)- | | | |
| | | 5-oxo-4,5-dihydro-1H-1,2,4- | | | |
| | | triazol-1-yl)-2-fluorophenoxy)-1,4- | | | |
| | | dimethyl-1H-pyrazol-3-yl) | | | |
| | | acetamide | | | |

### Example 144: Method 29

### 4-(2,6-difluorobenzyl)-2-(4-((4-(difluoromethyl)-2-(3-hydroxy-3-methylazetidin-1-yl)thiazol-5-yl)oxy)-3-fluorophenyl)-2,4-dihydro-3H-1,2,4- triazol-3-one

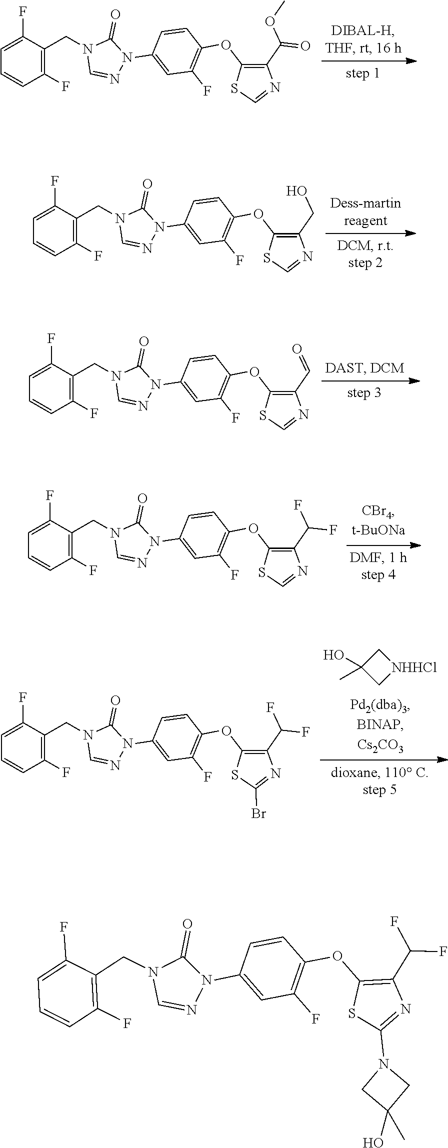

### Step 1: 4-(2,6-difluorobenzyl)-2-(3-fluoro-4-((4-(hydroxymethyl)thiazol-5-yl)oxy)phenyl)-2,4-dihydro-3H-1,2,4-triazol-3-one

To a stirred solution of methyl 5-(4-(4-(2,6-difluorobenzyl)-5-oxo-4,5-dihydro-1H-1,2,4-triazol-1-yl)-2-fluorophenoxy)thiazol e-4-carboxylate (4.13 g, 8.93 mmol) in THF (50 mL) was added Dibal-H (1.91 g, 13.4 mmol) at rt. The resulted mixture was stirred for 16 h at rt. The resulted mixture was diluted with water. The resulted mixture was extracted with EtOAc (3×100 mL). The combined organic layers were washed with brine, dried over anhydrous Na₂SO₄, and after filtration, the filtrate was concentrated under reduced pressure. The residue was purified by silica gel column chromatography, eluted with (PE:EtOAc) (1:1) to afford 4-(2,6-difluorobenzyl)-2-(3-fluoro-4-((4-(hydroxymethyl)thiazol-5-yl)oxy)phenyl)-2,4-dihydro-3H-1,2,4-triazol-3-one(1.12 g, 26% yield) as a yellow solid. MS (m/z): 435.0 [M+H]⁺.

### Step 2: 5-(4-(4-(2,6-difluorobenzyl)-5-oxo-4,5-dihydro-1H-1,2,4-triazol-1-yl)-2-fluorophenoxy)thiazole-4-carbaldehyde

To a stirred solution of 4-(2,6-difluorobenzyl)-2-(3-fluoro-4-((4-(hydroxymethyl)thiazol-5-yl)oxy)phenyl)-2,4-dihydro-3H-1,2,4-triazol-3-one(1.12 g, 2.59 mmol) in DCM (20 mL) was added Dess-martin (1.43 g, 3.36 mmol) at rt. The resulted mixture was stirred for 16 h at rt. The resulted mixture was diluted with water (30 mL). The resulted mixture was extracted with EtOAc (3×50 mL). The combined organic layers were washed with brine, dried over anhydrous Na₂SO₄, and after filtration, the filtrate was concentrated under reduced pressure. The residue was purified by silica gel column chromatography, eluted with (PE:EtOAc=3:1) to afford 5-(4-(4-(2,6-difluorobenzyl)-5-oxo-4,5-dihydro-1H-1,2,4-triazol-1-yl)-2-fluorophenoxy)thiazole-4-carbaldehyde (915 mg, 74% yield) as a yellow solid. MS (m/z): 433.0 [M+H]⁺.

### Step 3: 4-(2,6-difluorobenzyl)-2-(4-((4-(difluoromethyl)thiazol-5-yl)oxy)-3-fluorophenyl)-2,4-dihydro-3H-1,2,4-triazol-3-one

To a stirred solution of 5-(4-(4-(2,6-difluorobenzyl)-5-oxo-4,5-dihydro-1H-1,2,4-triazol-1-yl)-2-fluorophenoxy)thiazole-4-carbaldehyde (915 mg, 2.12 mmol) in DCM (10 mL) was added DAST (3.41 g, 21.2 mmol) at rt. The resulted mixture was stirred for 16 h at rt. The mixture was acidified to PH 8-9 with 1N NaHCO₃. The resulted mixture was extracted with EtOAc (3×20 mL). The combined organic layers were washed with brine, dried over anhydrous Na₂SO₄, and after filtration, the filtrate was concentrated under reduced pressure. The residue was purified by silica gel column chromatography, eluted with (PE:EtOAc=5:1) to afford 4-(2,6-difluorobenzyl)-2-(4-((4-(difluoromethyl)thiazol-5-yl)oxy)-3-fluorophenyl)-2,4-dihydro-3H-1,2,4-triazol-3-one (695 mg, 65% yield) as a yellow solid. LC-MS (m/z): 455.0 [M+H]⁺.

### Step 4: 2-(4-((2-bromo-4-(difluoromethyl)thiazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(4-((4-(difluoromethyl)thiazol-5-yl)oxy)-3-fluorophenyl)-2,4-dihydro-3H-1,2,4-triazol-3-one (695 mg, 1.53 mmol) and t-BuONa (441 mg, 4.59 mmol) in DMF (10 mL) was added CBr₄ (507 mg, 1.53 mmol) at rt. The resulted mixture was stirred for 16 h at rt. The resulted mixture was diluted with water (30 mL). The resulted mixture was extracted with EtOAc (3×50 mL). The combined organic layers were washed with brine, dried over anhydrous Na₂SO₄, and after filtration, the filtrate was concentrated under reduced pressure. The residue was purified by silica gel column chromatography, eluted with (PE:EtOAc=10:1) to afford 2-(4-((2-bromo-4-(difluoromethyl)thiazol-5-yl)oxy)-3-fluorophenyl)-4-(2,6-difluorobenzyl)-2,4-dihydro-3H-1,2,4-triazol-3-one (137 mg, 15% yield) as a yellow solid. LC-MS (m/z): 532.9, 534.9 [M+H]⁺.

### Step 5: 4-(2,6-difluorobenzyl)-2-(4-((4-(difluoromethyl)-2-(3-hydroxy-3-methylazetidin-1-yl)thiazol-5-yl)oxy)-3-fluorophenyl)-2,4-dihydro-3H-1,2,4-triazol-3-one

A mixture of 2-(4-((2-bromo-4-(difluoromethyl)thiazol-5-yl)oxy)-3-fluorophenyl)-4-(2,6-difluorobenzyl)-2,4-dihydro-3H-1,2,4-triazol-3-one(70 mg, 0.131 mmol), 3-methylazetidin-3-ol hydrochloride (24 mg, 0.197 mmol), Pd₂(dba)₃ (12 mg, 0.013 mmol), BINAP (16 mg, 0.026 mmol), and Cs₂CO₃ (64 mg, 0.197 mmol) in dioxane (10 mL) was stirred 16 h at 110° C. After cooling to rt, 30 mL of water was added. The resulted mixture was extracted with EtOAc (3×20 mL). The organic phases were combined, dried over Na₂SO₄, filtered and concentrated. The residue was purified by reverse phase flash chromatography with the following conditions: column: Spherical C18 40-60 um, 40 g; Mobile phase B: ACN; Flow rate: 40 mL/min; Gradient: 35% B-60% B in 20 min; Detector: 254 nm. The fractions containing the desired product were collected at 51% B and concentrated under reduced pressure to afford the titled compound (40 mg, 39.60 yield) as an off-white solid. LC-MS (m/z): 540.0 [M+H]⁺. ¹H NMR (400 MHz, CD₃OD_SPE) δ 8.01 (s, 1H), 7.87 (dd, J=12.4, 2.1 Hz, 1H), 7.74 (d, J=9.1 Hz, 1H), 7.50-7.40 (m, 11), 7.25 (t, J=9.0 Hz, 1H), 7.06 (t, J=8.0 Hz, 2H), 6.69 (t, J=53.5 Hz, 1H), 5.03 (s, 2H), 3.93 (d, J=3.7 Hz, 4H), 1.52 (s, 3H).

Examples (Compounds) 145-147 were synthesized using a similar method to that used in Example 144.

| Compound | | | Appear- | | |
|---|---|---|---|---|---|
| No. | Ac- | | ance | | MS(m/z) |
| Method | tivity | Structure and Name | and Yield | ¹H NMR Data | [M + H]⁺ |
| Example 144 Method 29 | ++++ | 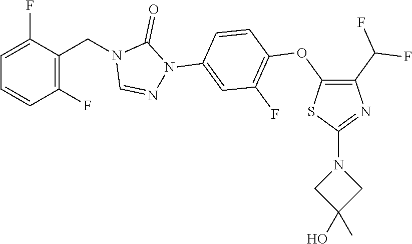 [SMILES: CC1(O)CN(c2nc(C(F)F)c(Oc3ccc(-n4ncn(Cc5c(F)cccc5F)c4=O)cc3F)s2)C1] | yellow solid, yield: 39.6% | ¹H NMR (400 MHz, CD₃OD) δ 8.01 (s, 1H), 7.87 (dd, J = 12.4, 2.1 Hz, 1H), 7.74 (d, J = 9.1 Hz, 1H), 7.50-7.40 (m, 1H), 7.25 (t, J = 9.0 Hz, 1H), 7.06 (t, J = 8.0 Hz, 2H), 6.69 (t, J = 53.5 Hz, 1H), 5.03 (s, 2H), 3.93 (d, J = 3.7 Hz, 4H), 1.52 (s, 3H). | 540.0 |
| | | 4-(2,6-difluorobenzyl)-2-(4-((4- | | | |
| | | (difluoromethyl)-2-(3-hydroxy- | | | |
| | | 3-methylazetidin-1-yl)thiazol-5- | | | |
| | | yl)oxy)-3-fluorophenyl)-2,4- | | | |
| | | dihydro-3H-1,2,4-triazol-3-one | | | |
| Example 145 Method 29 | ++++ | 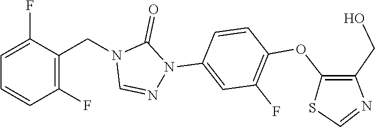 [SMILES: O=c1n(Cc2c(F)cccc2F)cnn1-c1ccc(Oc2scnc2CO)c(F)c1] | yellow solid, yield: 26% | NA | 435.0 |
| | | 4-(2,6-difluorobenzyl)-2-(3-fluoro- | | | |
| | | 4-((4-(hydroxymethyl)thiazol- | | | |
| | | 5-yl)oxy)phenyl)-2,4-dihydro- | | | |
| | | 3H-1,2,4-triazol-3-one | | | |
| Example 146 Method 29 | +++ | 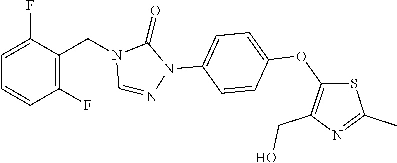 [SMILES: Cc1nc(CO)c(Oc2ccc(-n3ncn(Cc4c(F)cccc4F)c3=O)cc2)s1] | white solid, yield: 5.4% | ¹H NMR (400 MHz, CDCl₃) δ 7.94 (d, J = 8.9 Hz, 2H), 7.57 (s, 1H), 7.39-7.34 (m, 1H), 7.09 (d, J = 9.0 Hz, 2H), 7.00-6.96 (m, 2H), 4.97 (s, 2H), 4.64 (s, 2H), 2.68 (s, 3H). | 431.1 |
| | | 4-(2,6-difluorobenzyl)-2-(3-fluoro- | | | |
| | | 4-((4-(hydroxymethyl)-2-methyl- | | | |
| | | thiazol-5-yl)methyl)phenyl)- | | | |
| | | 2,4-dihydro-3H-1,2,4-triazol-3- | | | |
| | | one | | | |
| Example 147 Method 29 | ++++ | 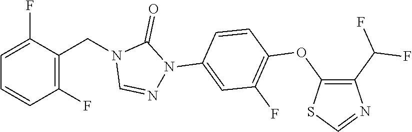 [SMILES: O=c1n(Cc2c(F)cccc2F)cnn1-c1ccc(Oc2scnc2C(F)F)c(F)c1] | yellow solid, yield: 74% | NA | 455.0 |
| | | 4-(2,6-difluorobenzyl)-2-(4-((4- | | | |
| | | (difluoromethyl)thiazol-5-yl)oxy)- | | | |
| | | 3-fluorophenyl)-2,4-dihydro- | | | |
| | | 3H-1,2,4-triazol-3-one | | | |

### Example 148: Method 30

### N-(2-aminoethyl)-5-(2-fluoro-4-(4-(2-fluorobenzyl)-5-oxo-4,5-dihydro-1H-1,2,4-triazol-1-yl)phenoxy)-4-methylthiazole-2-carboxamide

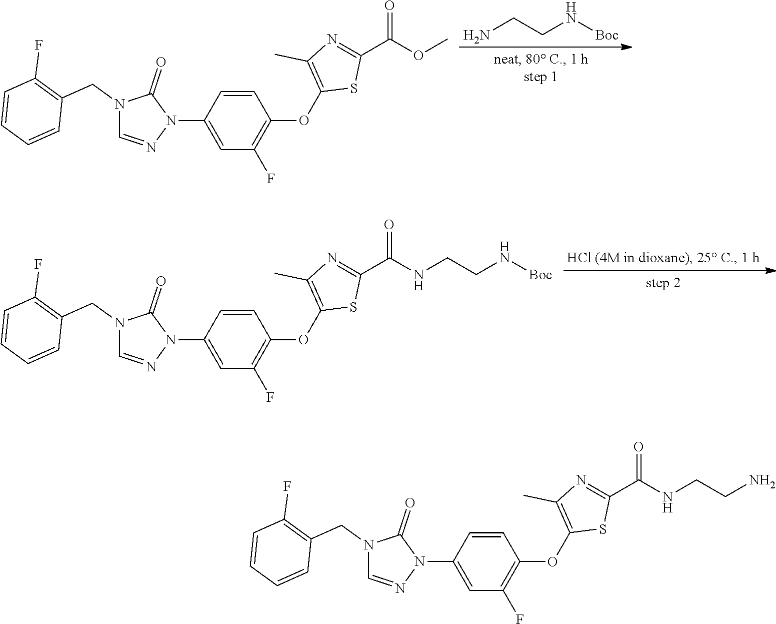

### Step 1: tert-butyl (2-(5-(2-fluoro-4-(4-(2-fluorobenzyl)-5-oxo-4,5-dihydro-1H-1,2,4-triazol-1-yl)phenoxy)-4-methylthiazole-2-carboxamido)ethyl)carbamate

A solution of methyl 5-(2-fluoro-4-(4-(2-fluorobenzyl)-5-oxo-4,5-dihydro-1H-1,2,4-triazol-1-yl)phenoxy)-4-methylthiazole-2-carboxylate (50 mg, 0.1 mmol) in tert-butyl (2-aminoethyl)carbamate (500 mg, 3.1 mmol) was stirred for 1 h at 80° C. The mixture was allowed to cool down to rt. The resulted mixture was diluted with water (30 mL). The resulted mixture was extracted with EtOAc (3×20 mL). The combined organic layers were washed with brine (30 mL), dried over anhydrous Na₂SO₄, and after filtration, the filtrate was concentrated under reduced pressure. The residue was purified by Prep-TLC, eluted with (EtOAc:PE=1:1) to afford the product (45 mg, 68.7% yield) as a white solid. MS (m/z): 531.1 [M−55]⁺. ¹H NMR (400 MHz, DMSO-d₆) δ 8.71 (t, J=5.8 Hz, 1H), 8.37 (s, 1H), 7.93 (dd, J=12.5, 2.4 Hz, 1H), 7.74 (d, J=9.2 Hz, 1H), 7.38 (dd, J=11.2, 7.1 Hz, 3H), 7.29-7.18 (m, 2H), 6.88 (s, 1H), 4.96 (s, 2H), 3.27 (d, J=6.5 Hz, 2H), 3.13-3.03 (m, 2H), 2.31 (s, 3H), 1.37 (s, 9H).

### Step 2: N-(2-aminoethyl)-5-(2-fluoro-4-(4-(2-fluorobenzyl)-5-oxo-4,5-dihydro-1H-1,2,4-triazol-1-yl)phenoxy)-4-methylthiazole-2-carboxamide

A solution of tert-butyl (2-(5-(2-fluoro-4-(4-(2-fluorobenzyl)-5-oxo-4,5-dihydro-1H-1,2,4-triazol-1-yl)phenoxy)-4-methylthiazole-2-carboxamido)ethyl)carbamate (35 mg, 0.06 mmol) in HCl (3 mL, 4M in dioxane) was stirred for 1 h at 25° C. The mixture was concentrated and purified by Prep-HPLC to afford the product (18 mg, 57% yield) as a white solid. MS (m/z): 487.0 [M+H]⁺. ¹H NMR (400 MHz, DMSO-d₆) δ 8.88 (t, J=5.8 Hz, 1H), 8.38 (s, 1H), 7.94 (dd, J=12.6, 2.3 Hz, 1H), 7.75 (d, J=9.1 Hz, 1H), 7.57 (s, 2H), 7.39 (dd, J=11.9, 6.2 Hz, 3H), 7.29-7.20 (m, 2H), 4.96 (s, 2H), 3.54-3.45 (m, 2H), 2.95 (t, J=6.0 Hz, 2H), 2.33 (s, 3H).

| Com- | | | Ap- | | |
|---|---|---|---|---|---|
| pound | | | pear- | | MS |
| No. | | | ance | | (m/z) |
| Meth- | Ac- | | and | | [M + |
| od | tivity | Structure and Name | Yield | ¹H NMR Data | H]⁺ |
| Exam- ple 148 Meth- od 30 | ++++ | 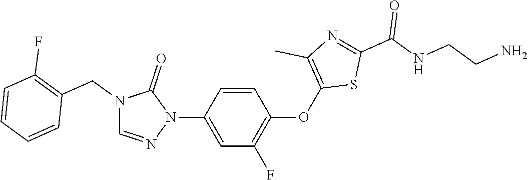 [SMILES: Cc1nc(C(=O)NCCN)sc1Oc1ccc(-n2ncn(Cc3ccccc3F)c2=O)cc1F] | white solid, yield: 57% | ¹H NMR (400 MHz, DMSO-d₆) δ 8.88 (t, J = 5.8 Hz, 1H), 8.38 (s, 1H), 7.94 (dd, J = 12.6, 2.3 Hz, 1H), 7.75 (d, J = 9.1 Hz, 1H), 7.57 (s, 2H), 7.39 (dd, J = 11.9, 6.2 Hz, 3H), 7.29-7.20 (m, 2H), 4.96 (s, 2H), 3.54-3.45 (m, 2H), 2.95 (t, J = 6.0 Hz, 2H), 2.33 (s, 3H). | 487.0 |
| | | N-(2-aminoethyl)-5-(2-fluoro-4- | | | |
| | | (4-(2-fluorobenzyl)-5-oxo-4,5- | | | |
| | | dihydro-1H-1,2,4-triazol-1-yl) | | | |
| | | phenoxy)-4-methylthiazole-2- | | | |
| | | carboxamide | | | |

### Example 149: Method 31

### (5S)-2-(3-fluoro-4-((2-(1-hydroxyethyl)-4-methylthiazol-5-yl)oxy)phenyl)-5-phenyl-2,5,6,7-tetrahydro-3H-pyrrolo[2,1-c][1,2,4]triazol-3-one

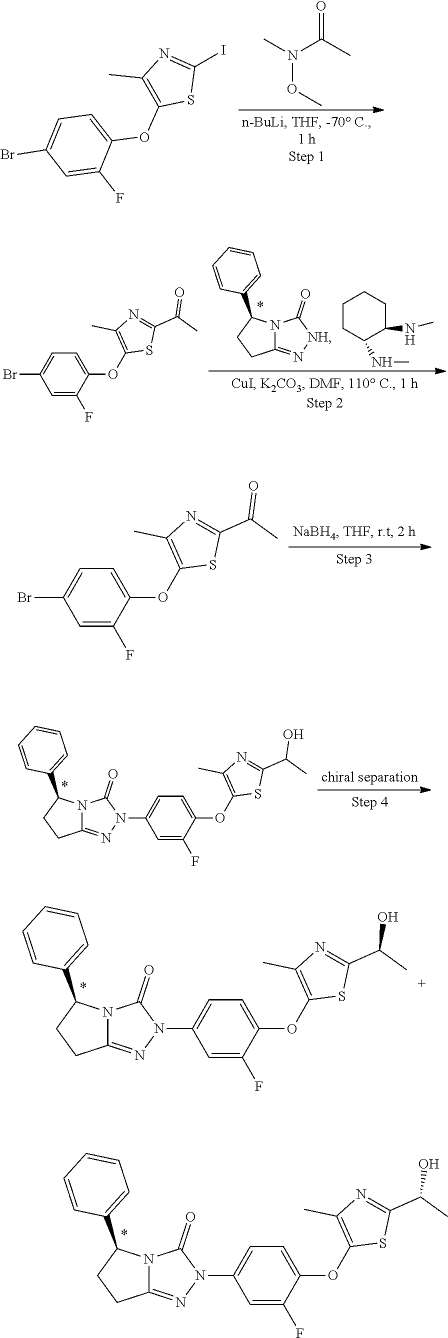

### Step 1: 1-(5-(4-bromo-2-fluorophenoxy)-4-methylthiazol-2-yl)ethan-1-one

To a stirred solution of 5-(4-bromo-2-fluorophenoxy)-2-iodo-4-methylthiazole (500 mg, 1.207 mol) in THF (70 mL) was added n-BuLi dropwise at −78° C. The resulted mixture was stirred for additional 20 mins at −78° C. Then the resulted mixture was added N-methoxy-N-methylacetamide (311.29 mg, 3.019 mol) at −78° C. The resulted mixture was stirred for additional 2h at −78° C. The reaction was then quenched by the addition of 300 mL of NH₄Cl. The resulted mixture was diluted with water (300 mL). The resulted mixture was extracted with EtOAc (3×400 mL). The combined organic layers were washed with brine (400 mL), dried over anhydrous Na₂SO₄, and after filtration, the filtrate was concentrated under reduced pressure. The residue was purified by flash (PE/EA=1:1) to afford 1-(5-(4-bromo-2-fluorophenoxy)-4-methylthiazol-2-yl)ethan-1-one (232 mg, 57.5% yield) as yellow oil. MS (m/z): 330.2 [M+H]⁺.

### Step 2: (S)-2-(4-((2-acetyl-4-methylthiazol-5-yl)oxy)-3-fluorophenyl)-5-phenyl-2,5,6,7-tetrahydro-3H1-pyrrolo[2,1-c][1,2,4]triazol-3-one

To a stirred solution of 1-(5-(4-bromo-2-fluorophenoxy)-4-methylthiazol-2-yl)ethan-1-one (60 mg, 0.1817 mol) and (S)-5-phenyl-2,5,6,7-tetrahydro-3H-pyrrolo[2,1-c][1,2,4]triazol-3-one [2,1-c][1,2,4]triazol-3-one (100 mg, 0.497 mmol) and DCHDMA(11.33 mg, 0.099 mmol), was added CuI (18.9 mg, 0.0994 mmol), and K₂CO₃ (171.72 mg, 1.2425 mmol) in DMF (5 mL) at rt. The resulted mixture was stirred for additional 1 h at 110° C. under N₂. After filtration, the filtrate was concentrated under reduced pressure. The resulted mixture was diluted with water (100 mL). The resulted mixture was extracted with EtOAc (1×400 mL). The combined Organic layers were washed with brine (100 mL), dried over anhydrous Na₂SO₄, and after filtration, the filtrate was concentrated under reduced pressure. The residue was purified by flash (PE/EA=1:1) to afford the desired product (50 mg, 54.65% yield) as a yellow oil. MS (m/z): 451.1[M+H]⁺.

### Step 3: (5S)-2-(3-fluoro-4-((2-(1-hydroxyethyl)-4-methylthiazol-5-yl)oxy)phenyl)-5-phenyl-2,5,6,7-tetrahydro-3H-pyrrolo[2,1-c][1,2,4]triazol-3-one

To a stirred solution of (S)-2-(4-((2-acetyl-4-methylthiazol-5-yl)oxy)-3-fluorophenyl)-5-phenyl-2,5,6,7-tetrahydro-3H-pyrrolo[2,1-c][1,2,4]triazol-3-one (50 mg, 0.111 mol) in THF (2 mL) was added NaBH₄ (8.39 mg, 0.2217 mol) at 0° C. The resulted mixture was stirred for additional 10 min at 0° C. under nitrogen. The mixture was added CuBr₂(4.05 g, 0.0181 mol) at 0° C. The resulted mixture was stirred for additional 34 hr at RT under nitrogen. The reaction was then quenched by the addition of 10 mL of NH₄Cl. The resulted mixture was extracted with EtOAc (3×60 mL). The combined organic layers were washed with brine (90 mL), dried over anhydrous Na₂SO₄, and after filtration, the filtrate was concentrated under reduced pressure. The residue was purified by silica gel column chromatography, eluted with (PE:EtOAc) (3:1) to get the titled compound (20 mg, 27.6% yield) as a white solid. LC-MS (m/z) 453.1 [M+H]⁺. ¹H NMR (400 MHz, CDCl₃) δ 7.87 (d, J=12.4 Hz, 1H), 7.69 (d, J=8.8 Hz, 1H), 7.41-7.32 (m, 3H), 7.24 (s, 2H), 7.00 (t, J=8.4 Hz, 1H), 5.31-5.24 (m, 1H), 5.01 (d, J=5.6 Hz, 1H), 3.11-2.86 (m, 4H), 2.30 (s, 3H), 1.60 (d, J=4.8 Hz, 3H). The racemic compound was purified by chiral separation to give two compounds:

### Peak-1 (S)-2-(3-fluoro-4-((2-((R)-1-hydroxyethyl)-4-methylthiazol-5-yl)oxy)phenyl)-5-phenyl-2,5,6,7-tetrahydro-3H-pyrrolo[2,1-c][1,2,4]triazol-3-one (single unknown stereoisomer)

¹H NMR (400 MHz, CDCl₃) δ 7.87 (dd, J=12.4, 2.0 Hz, 1H), 7.68 (d, J=9.2 Hz, 1H), 7.46-7.26 (m, 5H), 6.99 (t, J=9.2 Hz, 1H), 5.36-5.23 (m, 1H), 4.99 (q, J=6.4 Hz, 1H), 3.16-2.83 (m, 3H), 2.68-2.45 (m, 1H), 2.29 (s, 3H), 1.59 (d, J=6.8 Hz, 3H).

peak-2:

### (S)-2-(3-fluoro-4-((2-((S)-1-hydroxyethyl)-4-methylthiazol-5-yl)oxy)phenyl)-5-phenyl-2,5,6,7-tetrahydro-3H-pyrrolo[2,1-c][1,2,4]triazol-3-one (single unknown stereoisomer)

¹H NMR (400 MHz, CDCl₃) δ 7.87 (dd, J=12.4, 2.0 Hz, 1H), 7.68 (d, J=9.2 Hz, 1H), 7.46-7.26 (m, 5H), 6.99 (t, J=9.2 Hz, 1H), 5.36-5.23 (m, 1H), 4.99 (q, J=6.4 Hz, 1H), 3.16-2.83 (m, 3H), 2.68-2.45 (m, 1H), 2.29 (s, 3H), 1.59 (d, J=6.8 Hz, 3H).

Examples (Compounds) 150-151 were synthesized using a similar method to that used in Example 149.

| Compound | | | Appear- | | |
|---|---|---|---|---|---|
| No. | | | ance | | MS(m/z) |
| Method | Activity | Structure and Name | and Yield | ¹H NMR Data | [M + H]⁺ |
| Exam- ple 149 Method 31 single unknown stereo- isomer | ++++ | 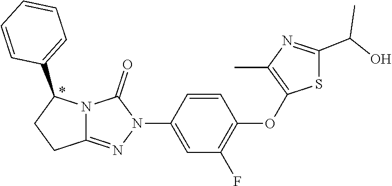 [SMILES: Cc1nc(C(C)O)sc1Oc1ccc(-n2nc3n(c2=O)[C@H](c2ccccc2)CC3)cc1F] | white solid, yield: 27.6% | ¹H NMR (400 MHz, CDCl₃) δ 7.87 (d, J = 12.4 Hz, 1H), 7.69 (d, J = 8.8 Hz, 1H), 7.41-7.32 (m, 3H), 7.24 (s, 2H), 7.00 (t, J = 8.4 Hz, 1H), 5.31-5.24 (m, 1H), 5.01 (d, J = 5.6 Hz, 1H), 3.11- 2.86 (m, 4H), 2.30 (s, 3H), 1.60 (d, J = 4.8 Hz, 3H). | 453.1 |
| | | (5S)-2-(3-fluoro-4-((2-(1-hydroxy- | | | |
| | | ethyl)-4-methylthiazol-5-yl) | | | |
| | | oxy)phenyl)-5-phenyl-2,5,6,7- | | | |
| | | tetrahydro-3H-pyrrolo[2,1-c][1,2,4] | | | |
| | | triazol-3-one | | | |
| Exam- ple 150 Method 31 single unknown stereo- isomer | ++++ | 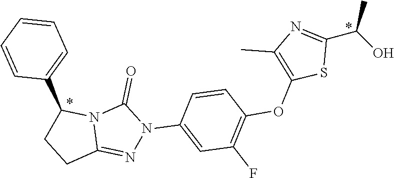 [SMILES: Cc1nc([C@@H](C)O)sc1Oc1ccc(-n2nc3n(c2=O)[C@H](c2ccccc2)CC3)cc1F] | white solid, yield: 40% | ¹H NMR (400 MHz, CDCl₃) δ 7.87 (dd, J = 12.4, 2.0 Hz, 1H), 7.68 (d, J = 9.2Hz, 1H), 7.46-7.26 (m, 5H), 6.99 (t, J = 9.2 Hz, 1H), 5.36-5.23 (m, 1H), 4.99 (q, J = 6.4 Hz, 1H), 3.16-2.83 (m, 3H), 2.68-2.45 (m, 1H), 2.29 (s, 3H), 1.59 (d, J = 6.8 Hz, 3H). | 453.1 |
| | | (S)-2-(3-fluoro-4-((2-((R)-1-hydroxy- | | | |
| | | ethyl)-4-methylthiazol-5-yl) | | | |
| | | oxy)phenyl)-5-phenyl-2,5,6,7- | | | |
| | | tetrahydro-3H-pyrrolo[2,1-c] | | | |
| | | [1,2,4]triazol-3-one | | | |
| | | Unknown absolute | | | |
| | | configuration | | | |
| Exam- ple 151 Method 31 single unknown stereo- isomer | ++++ | 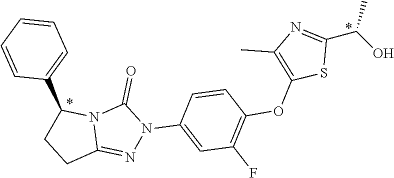 [SMILES: Cc1nc([C@H](C)O)sc1Oc1ccc(-n2nc3n(c2=O)[C@H](c2ccccc2)CC3)cc1F] | white solid, yield: 42% | ¹H NMR (400 MHz, CDCl₃) δ 7.87 (dd, J = 12.4, 2.0 Hz, 1H), 7.68 (d, J = 9.2Hz, 1H), 7.46-7.26 (m, 5H), 6.99 (t, J = 9.2 Hz, 1H), 5.36-5.23 (m, 1H), 4.99 (q, J = 6.4 Hz, 1H), 3.16-2.83 (m, 3H), 2.68-2.45 (m, 1H), 2.29 (s, 3H), 1.59 (d, J = 6.8 Hz, 3H). | 453.1 |
| | | (S)-2-(3-fluoro-4-((2-((S)-1- | | | |
| | | hydroxyethyl)-4-methylthiazol-5-yl) | | | |
| | | oxy)phenyl)-5-phenyl-2,5,6,7- | | | |
| | | tetrahydro-3H-pyrrolo[2,1-c] | | | |
| | | [1,2,4]triazol-3-one | | | |
| | | Unknown absolute | | | |
| | | configuration | | | |

### 4-(2,6-difluorobenzyl)-2-(4-((5-(difluoromethyl)thiazol-4-yl)oxy)-3-fluorophenyl)-2,4-dihy dro-3H-1,2,4-triazol-3-one

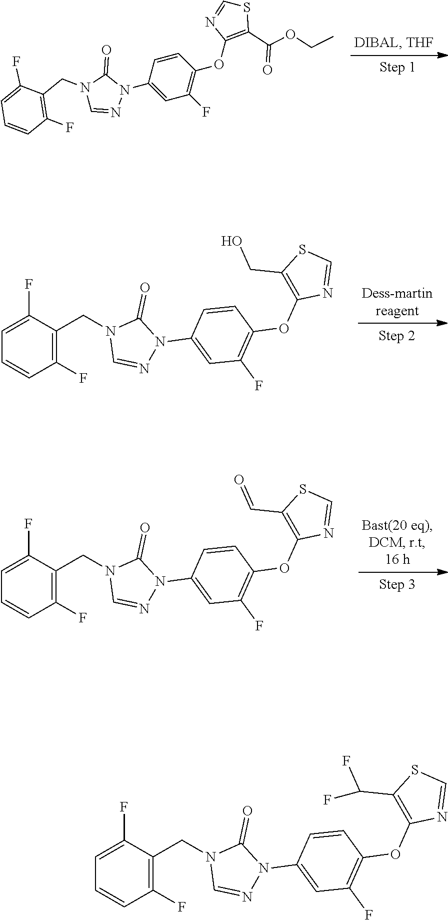 [SMILES: CCOC(=O)c1scnc1Oc1ccc(-n2ncn(Cc3c(F)cccc3F)c2=O)cc1F ; O=c1n(Cc2c(F)cccc2F)cnn1-c1ccc(Oc2ncsc2CO)c(F)c1 ; O=Cc1scnc1Oc1ccc(-n2ncn(Cc3c(F)cccc3F)c2=O)cc1F ; O=c1n(Cc2c(F)cccc2F)cnn1-c1ccc(Oc2ncsc2C(F)F)c(F)c1]

### Step 1. 4-(2,6-difluorobenzyl)-2-(3-fluoro-4-((5-(hydroxymethyl)thiazol-4-yl)oxy)phenyl)-2,4-dihydro-3H-1,2,4-triazol-3-one

Diisobutylaluminium hydride in hexane (1 M, 0.76 mL) was added to a solution of ethyl 4-(4-{4-[(2,6-difluorophenyl)methyl]-5-oxo-1,2,4-triazol-1-yl}-2-fluorophenoxy)-1,3-thiazole-5-carboxylate (180 mg, 0.38 mmol) in THF (5 mL) at 0° C. The reaction mixture was stirred at 0° C. for 1h, and water (1 mL) was added. The resulted reaction mixture was filtrated and the solvent was removed under vacuum and the crude was purified through silica gel chromatography (PE: EA=3:1) to give the desired product as a yellow solid (130 mg, 79% yield). LCMS (m/z): 435.0 [M+H]⁺.

### Step 2. 4-(4-(4-(2,6-difluorobenzyl)-5-oxo-4,5-dihydro-1H-1,2,4-triazol-1-yl)-2-fluorophenoxy)thiazole-5-carbaldehyde

Dess Martin reagent (176 mg, 0.41 mmol) was added to a solution of 4-[(2,6-difluorophenyl)methyl]-2-(3-fluoro-4-{[5-(hydroxymethyl)-1,3-thiazol-4-yl]oxy}pheny 1)-1,2,4-triazol-3-one (120 mg, 0.28 mmol) in DCM (10 mL). The mixture was stirred at rt for 1h, then filtrated, and the solvent was removed under vacuum to give the desired product as a yellow solid (120 mg, 100%). LCMS (m/z): 433.0 [M+H]⁺, RT: 1.305 min.

### Step 3. 4-(2,6-difluorobenzyl)-2-(4-((5-(difluoromethyl)thiazol-4-yl)oxy)-3-fluorophenyl)-2,4-dihydro-3H-1,2,4-triazol-3-one

To a solution of 4-(4-{4-[(2,6-difluorophenyl)methyl]-5-oxo-1,2,4-triazol-1-yl}-2-fluorophenoxy)-1,3-thiazole-5-carbaldehyde (30 mg, 0.069 mmol) in DCM (5 mL) was added DAST (224 mg, 1.39 mmol). The mixture was stirred at rt for 16h, diluted with water (30 mL), and extracted three times with ethyl acetate (20 mL). The organic layers were combined, the solvent was removed under vacuum, and the crude was purified by Prep-HPLC (column-Gemini-C18 150×21.2 mm, Sum; Mobile phase: ACN-H₂O (0.1% FA), 5%-20%) to afford the desired product as a white solid (4.7 mg, 14.9%). LCMS (m/z): 455.0 [M+H]⁺. ¹H NMR (400 MHz, DMSO-d₆) δ 8.60 (s, 1H), 7.93 (dd, J=12.1, 2.3 Hz, 1H), 7.82 (d, J=9.0 Hz, 1H), 7.58 (s, 1H), 7.37 (t, J=7.5 Hz, 2H), 7.10-6.95 (m, 3H), 4.97 (s, 2H).

| Compound No. | | | Appearance | | MS(m/z) |
|---|---|---|---|---|---|
| Method | Activity | Structure and Name | and Yield | ¹H NMR Data | [M + H]⁺ |
| Example 152 Method 32 | ++++ | 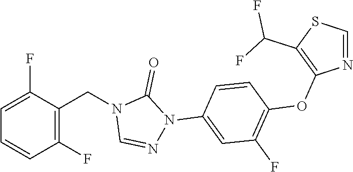 [SMILES: O=c1n(Cc2c(F)cccc2F)cnn1-c1ccc(Oc2ncsc2C(F)F)c(F)c1] | white solid, yield: 14.9% | ¹H NMR (400 MHz, DMSO-d₆) δ 8.60 (s, 1H), 7.93 (dd, J = 12.1, 2.3 Hz, 1H), 7.82 (d, J = 9.0 Hz, 1H), 7.58 (s, 1H), 7.37 (t, J = 7.5 Hz, 2H), 7.10-6.95 (m, 3H), 4.97 (s, 2H). | 455.0 |
| | | 4-(2,6-difluorobenzyl)-2-(4-((5- | | | |
| | | (difluoromethyl)thiazol-4-yl)oxy)- | | | |
| | | 3-fluorophenyl)-2,4-dihydro- | | | |
| | | 3H-1,2,4-triazol-3-one | | | |

### Example 153: Method 33

### 2-(4-((2-(3-amino-3-methylazetidin-1-yl)-4-(difluoromethyl)thiazol-5-yl)oxy)-3-fluorophen yl)-4-(2,6-difluorobenzyl)-2,4-dihydro-3H-1,2,4-triazol-3-one

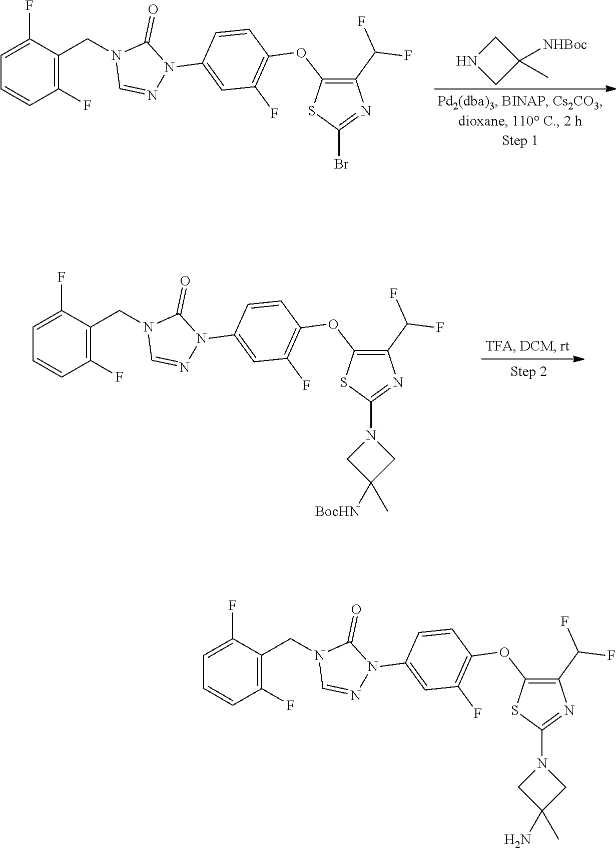 [SMILES: O=c1n(Cc2c(F)cccc2F)cnn1-c1ccc(Oc2sc(Br)nc2C(F)F)c(F)c1 ; CC1(NC(=O)OC(C)(C)C)CNC1 ; CC1(NC(=O)OC(C)(C)C)CN(c2nc(C(F)F)c(Oc3ccc(-n4ncn(Cc5c(F)cccc5F)c4=O)cc3F)s2)C1 ; CC1(N)CN(c2nc(C(F)F)c(Oc3ccc(-n4ncn(Cc5c(F)cccc5F)c4=O)cc3F)s2)C1]

### Step 1: tert-butyl (1-(5-(4-(4-(2,6-difluorobenzyl)-5-oxo-4,5-dihydro-1H-1,2,4-triazol-1-yl)-2-fluorophenoxy)-4-(difluoromethyl)thiazol-2-yl)-3-methylazetidin-3-yl)carbamate

A mixture solution of 2-(4-((2-bromo-4-(difluoromethyl)thiazol-5-yl)oxy)-3-fluorophenyl)-4-(2,6-difluorobenzyl)-2,4-dihydro-3H-1,2,4-triazol-3-one (60 mg, 0.11 mmol), tert-butyl (3-methylazetidin-3-yl)carbamate (37 mg, 0.17 mmol), Cs₂CO₃ (110 mg, 0.34 mmol), BINAP(14 mg, 0.022 mmol), Pd₂(dba)₃ (21 mg, 0.022 mmol), and dioxane (10 mL) was stirred at 110° C. for 3 hrs. Then the reaction mixture was cooled to room temperature, diluted with water (30 mL), and extracted three times with ethyl acetate (20 mL). The organic layers were combined, the solvent was removed under vacuum, and the crude was purified through silica gel chromatography (PE: EA=7:1) to give the desired product as a yellow solid (30 mg, yield: 41.9%). LCMS (m/z): 639.1 [M+H]⁺, RT: 1.428 min.

### Step 2: 2-(4-((2-(3-amino-3-methylazetidin-1-yl)-4-(difluoromethyl)thiazol-5-yl)oxy)-3-fluorophenyl)-4-(2,6-difluorobenzyl)-2,4-dihydro-3H-1,2,4-triazol-3-one

To a solution of tert-butyl {1-[4-(difluoromethyl)-5-(4-{4-[(2,6-difluorophenyl)methyl]-5-oxo-1,2,4-triazol-1-yl}-2-fluorophenoxy)-1,3-thiazol-2-yl]-3-methylazetidin-3-yl}amino formate(4) (30 mg, 0.047 mmol) in DCM(10 mL) was added TFA(2 mL). The mixture was stirred at rt for 2 h, the solvent was removed under vacuum, and the crude was purified by Prep-HPLC (column-Gemini-C18 150×21.2 mm, Sum; Mobile phase: ACN-H₂O (0.10% FA), 5%-˜20%) to afford the desired product as a white solid (4.1 mg, 16.2% yield). LCMS (m-z): 539.1 [M+H]⁺, RT: 1.098 min. ¹H NMR (400 MHz, DMSO-d₆) δ 8.36 (s, 1H), 8.30 (s, 2H), 7.88 (dd, J=12.6, 2.3 Hz, 1H), 7.72 (d, J=9.3 Hz, 1H), 7.48 (t, J 7.6 Hz, 1H), 7.33 (t, J 9.1 Hz, 1H), 7.16 (t, J=8.0 Hz, 2H), 6.97 (t, J=53.0 Hz, 1H), 4.98 (s, 2H), 4.02 (dd, J=33.8, 8.9 Hz, 4H), 1.56 (s, 3H).

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 153 Method 33 | ++++ | 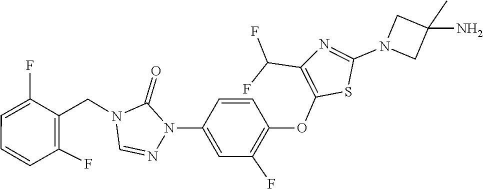 [SMILES: CC1(N)CN(c2nc(C(F)F)c(Oc3ccc(-n4ncn(Cc5c(F)cccc5F)c4=O)cc3F)s2)C1] 2-(4-((2-(3-amino-3-methylazetidin- 1-yl)-4-(difluoromethyl)thiazol- 5-yl)oxy)-3-fluorophenyl)-4- (2,6-difluorobenzyl)-2,4-dihydro- 3H-1,2,4-triazol-3-one | white solid, yield: 16.2% | ¹H NMR (400 MHz, DMSO-d₆) δ 8.36 (s, 1H), 8.30 (s, 2H), 7.88 (dd, J = 12.6, 2.3 Hz, 1H), 7.72 (d, J = 9.3 Hz, 1H), 7.48 (t, J = 7.6 Hz, 1H), 7.33 (t, J = 9.1 Hz, 1H), 7.16 (t, J = 8.0 Hz, 2H), 6.97 (t, J = 53.0 Hz, 1H), 4.98 (s, 2H), 4.02 (dd, J = 33.8, 8.9 Hz, 4H), 1.56 (s, 3H). | 539.1 |

### Example 154: Method 34

### 4-(2,6-difluorobenzyl)-2-(3-fluoro-4-((2-(2-hydroxy-2-methylpropyl)-4-methylthiazol-5-yl)oxy)phenyl)-2,4-dihydro-3H-1,2,4-triazol-3-one

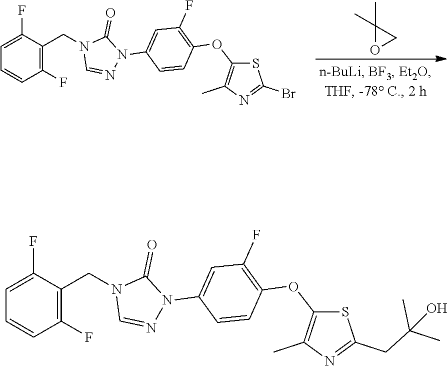

To a stirred solution of 2-(4-((2-bromo-4-methylthiazol-5-yl)oxy)-3-fluorophenyl)-4-(2,6-difluorobenzyl)-2,4-dihydro-3H-1,2,4-triazol-3-one (100 mg, 0.20 mmol) in THF (5 mL) at −78° C., under N₂ atmosphere, was added n-BuLi in THF solution (0.08 mL, 2.5M, 0.20 mmol). Then the mixture solution was stirred at −78° C. for 20 min, and was then successively added 2,2-dimethyloxirane (23 mg, 0.30 mmol) and BF₃·Et₂O (43 mg, 0.30 mmol). The resulted mixture solution was stirred at −78° C. for 100 min. The reaction was quenched with a saturated NH₄Cl solution and was diluted with EtOAc (10 mL), the solution was washed with water (10 mL) and brine (10 mL), dried over anhydrous Na₂SO₄, and after filtration, the filtrate was concentrated under reduced pressure. The residue was purified by TLC (PE/EtOAc) (1:1) to afford the titled compound as a white solid (14 mg, yield: 14.3%). MS (m/z): 491.2 [M+H]⁺. ¹H NMR (400 MHz, CDCl₃) δ 7.94-7.86 (m, 1H), 7.79-7.69 (m, 1H), 7.59-7.49 (m, 1H), 7.44-7.32 (m, 2H), 7.13-6.78 (m, 3H), 4.97 (s, 2H), 2.78 (s, 2H), 2.06 (s, 3H), 1.26 (s, 6H).

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 154 Method 34 | ++++ | 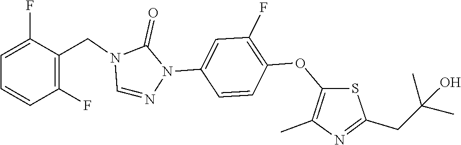 [SMILES: Cc1nc(CC(C)(C)O)sc1Oc1ccc(-n2ncn(Cc3c(F)cccc3F)c2=O)cc1F] 4-(2,6-difluorobenzyl)-2-(3-fluoro- 4-((2-(2-hydroxy-2-methylpropyl)- 4-methylthiazol-5-yl)oxy) phenyl)-2,4-dihydro-3H-1,2,4- triazol-3-one | white solid, yield: 14.3% | ¹H NMR (400 MHz, CDCl₃) δ 7.94-7.86 (m, 1H), 7.79-7.69 (m, 1H), 7.59-7.49 (m, 1H), 7.44-7.32 (m, 2H), 7.13-6.78 (m, 3H), 4.97 (s, 2H), 2.78 (s, 2H), 2.06 (s, 3H), 1.26 (s, 6H). | 491.2 |

### Example 155: Method 35

### 2-(5-(4-(4-(2,6-difluorobenzyl)-5-oxo-4,5-dihydro-1H-1,2,4-triazol-1-yl)-2-fluorophenoxy)-4-methylthiazol-2-yl)acetonitrile

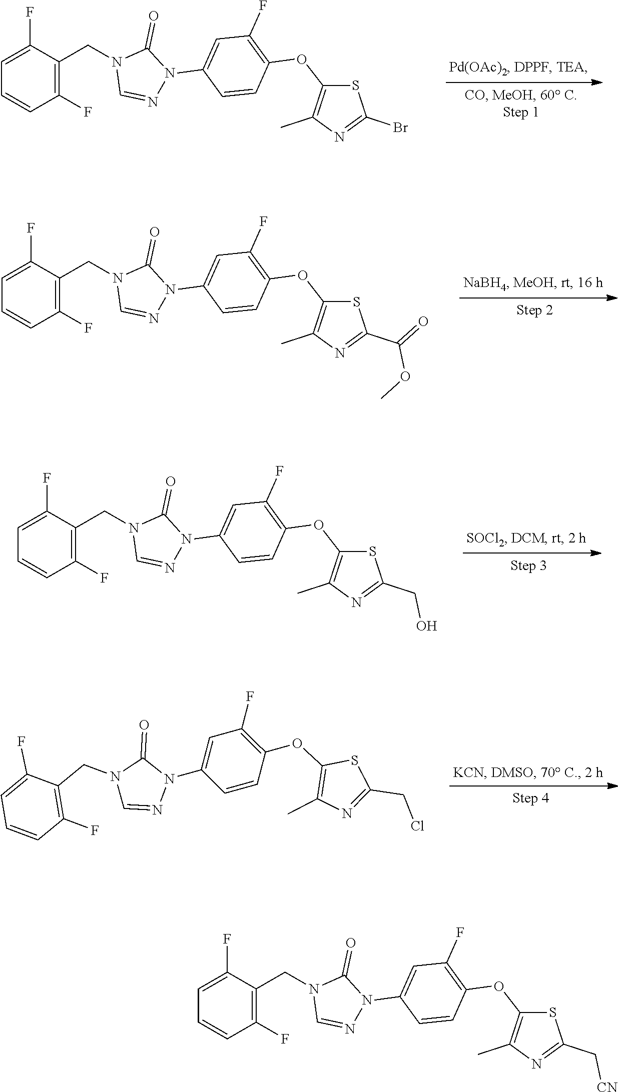 [SMILES: Cc1nc(Br)sc1Oc1ccc(-n2ncn(Cc3c(F)cccc3F)c2=O)cc1F ; COC(=O)c1nc(C)c(Oc2ccc(-n3ncn(Cc4c(F)cccc4F)c3=O)cc2F)s1 ; Cc1nc(CO)sc1Oc1ccc(-n2ncn(Cc3c(F)cccc3F)c2=O)cc1F ; Cc1nc(CCl)sc1Oc1ccc(-n2ncn(Cc3c(F)cccc3F)c2=O)cc1F ; Cc1nc(CC#N)sc1Oc1ccc(-n2ncn(Cc3c(F)cccc3F)c2=O)cc1F]

### Step 1: methyl5-(4-(4-(2,6-difluorobenzyl)-5-oxo-4,5-dihydro-1H-1,2,4-triazol-1-yl)-2-fluorophenoxy)-4-methylthiazole-2-carboxylate

To a stirred solution of 2-(4-((2-bromo-4-methylthiazol-5-yl)oxy)phenyl)-4-(2,6-difluorobenzyl)-2,4-dihydro-3H-1,2,4-triazol-3-one (1.2 g, 2.5 mmol), Pd(OAc)₂ (56 mg, 0.25 mmol), DPPF (138.5 mg, 0.25 mmol), TEA (757.5 mg, 7.5 mmol) in MeOH (50 mL) under CO. The resulted mixture was stirred for additional 12 h at 70′C. The resulted mixture was filtered and extracted with EtOAc. The combined organic layers was dried over anhydrous Na₂SO₄, after filtration, the filtrate was concentrated under reduced pressure to afford methyl 5-(4-(4-(2,6-difluorobenzyl)-5-oxo-4,5-dihydro-1H-1,2,4-triazol-1-yl)-2-fluorophenoxy)-4-met hylthiazole-2-carboxylate (960 mg, 40.0% yield) as a white solid. LC-MS (m/z): 477.1[M+H]⁺.

### Step 2: methyl5-(4-(4-(2,6-difluorobenzyl)-5-oxo-4,5-dihydro-1H-1,2,4-triazol-1-yl)-2-fluorophenoxy)-4-methylthiazole-2-carboxylate

To a stirred solution of methyl 5-(4-(4-(2,6-difluorobenzyl)-5-oxo-4,5-dihydro-1H-1,2,4-triazol-1-yl)-2-fluorophenoxy)-4-methylthiazole-2-carboxylate (340 mg, 0.71 mmol) in MeOH (20 mL) was added NaBH4 (269.8 mg, 7.1 mmol). The resulted mixture was stirred for additional 1 h at 25° C. The resulted mixture was diluted with water (20 mL). The resulted mixture was extracted with EtOAc (3×30 mL). The combined Organic layers were dried over anhydrous Na₂SO₄, and after filtration, the filtrate was concentrated under reduced pressure and afford 4-(2,6-difluorobenzyl)-2-(3-fluoro-4-((2-(hydroxymethyl)-4-methylthiazol-5-yl)oxy)phenyl)-2,4-dihydro-3H-1,2,4-triazol-3-one (210 mg, 66.0% yield) as a white solid. LC-MS (m/z): 449.1[M+H]⁺.

### Step 3: 2-(4-((2-(chloromethyl)-4-methylthiazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(3-fluoro-4-((2-(hydroxymethyl)-4-methylthiazol-5-yl)oxy)phenyl)-2,4-dihydro-3H-1,2,4-triazol-3-one (100 mg, 0.22 mmol) in DCM was added SOCl2 (20 mL) at room temperature. The resulted mixture was stirred for additional 1 h at 70′C. The filtrate was concentrated under reduced pressure to afford 2-(4-((2-(chloromethyl)-4-methylthiazol-5-yl)oxy)-3-fluorophenyl)-4-(2,6-difluorobenzyl)-2,4-dihydro-3H-1,2,4-triazol-3-one (60 mg, 58.8% yield) as a white solid. LC-MS (m/z): 467.1 [M+H]⁺.

### Step 4: 2-(5-(4-(4-(2,6-difluorobenzyl)-5-oxo-4,5-dihydro-1H-1,2,4-triazol-1-yl)-2-fluorophenoxy)-4-methylthiazol-2-yl)acetonitrile

To a stirred solution of 2-(4-((2-(chloromethyl)-4-methylthiazol-5-yl)oxy)-3-fluorophenyl)-4-(2,6-difluorobenzyl)-2,4-dihydro-3H-1,2,4-triazol-3-one (60 mg, 0.13 mmol) in DMSO (20 mL) was KCN (25.3 mg, 0.39 mmol). The resulted mixture was stirred for additional 2 h at 70° C. The resulted mixture was diluted with water (20 mL). The resulted mixture was extracted with EtOAc (3×30 mL). The combined organic layers was dried over anhydrous Na₂SO₄, and after filtration, the filtrate was concentrated under reduced pressure to afford 2-(5-(4-(4-(2,6-difluorobenzyl)-5-oxo-4,5-dihydro-1H-1,2,4-triazol-1-yl)-2-fluorophenoxy)-4-methylthiazol-2-yl)acetonitrile (4.7 mg, 7.9% yield) as a white solid. LC-MS (m/z): 458.1[M+H]⁺.

¹H NMR NMR (400 MHz, CDCl₃) δ 7.91 (dd, J=12.2, 2.4 Hz, 1H), 7.73 (m, 1H), 7.58 (s, 1H), 7.36 (ddd, J=14.9, 8.3, 6.7 Hz, 1H), 6.99 (dd, J=17.6, 8.7 Hz, 3H), 4.96 (s, 2H), 3.98 (s, 2H), 2.29 (s, 3H).

Examples (Compounds) 156-158 were synthesized using a similar method to that used in Example 155.

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 155 Method 35 | ++++ | 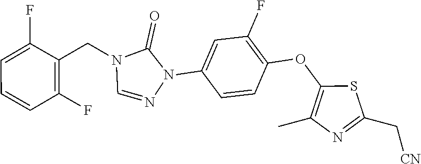 [SMILES: Cc1nc(CC#N)sc1Oc1ccc(-n2ncn(Cc3c(F)cccc3F)c2=O)cc1F] 2-(5-(4-(4-(2,6-difluorobenzyl)- 5-oxo-4,5-dihydro-1H-1,2,4- triazol-1-yl)-2-fluorophenoxy)-4- methylthiazol-2-yl)acetonitrile | white solid, yield: 7.9% | ¹H NMR NMR (400 MHz, CDCl₃) δ 7.91 (dd, J = 12.2, 2.4 Hz, 1H), 7.73 (m, 1H), 7.58 (s, 1H), 7.36 (ddd, J = 14.9, 8.3, 6.7 Hz, 1H), 6.99 (dd, J = 17.6, 8.7 Hz, 3H), 4.96 (s, 2H), 3.98 (s, 2H), 2.29 (s, 3H). | 458.1 |
| Example 156 Method 35 | ++++ | 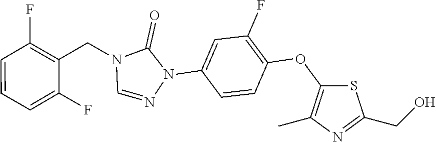 [SMILES: Cc1nc(CO)sc1Oc1ccc(-n2ncn(Cc3c(F)cccc3F)c2=O)cc1F] 4-(2,6-difluorobenzyl)-2-(3-fluoro- 4-((2-(hydroxymethyl)-4- methylthiazol-5-yl)oxy)phenyl)-2,4- dihydro-3H-1,2,4-triazol-3-one | white solid, yield: 45.1% | ¹H NMR (400 MHz, DMSO-d₆) δ 8.40 (s, 1H), 7.91-7.80 (m, 2H), 7.48 (tt, J = 8.4, 6.8 Hz, 1H), 7.19- 7.09 (m, 4H), 4.96 (s, 2H), 4.31 (s, 2H), 2.22 (s, 3H). | 449.0 |
| Example 157 Method 35 | ++++ | 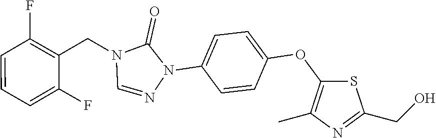 [SMILES: Cc1nc(CO)sc1Oc1ccc(-n2ncn(Cc3c(F)cccc3F)c2=O)cc1] 4-(2,6-difluorobenzyl)-2-(4-((2- (hydroxymethyl)-4-methylthiazol- 5-yl)oxy)phenyl)-2,4-dihydro- 3H-1,2,4-triazol-3-one | white solid, yield: 55.9% | ¹H NMR (400 MHz, DMSO-d₆) δ 8.29 (d, J = 0.8 Hz, 1H), 7.88- 7.80 (m, 2H), 7.48 (tt, J = 8.4, 6.8 Hz, 1H), 7.21-7.08 (m, 4H), 6.02 (t, J = 6.0 Hz, 1H), 4.97 (s, 2H), 4.60 (d, J = 6.0 Hz, 2H), 2.14 (s, 3H). | 431.0 |
| Example 158 Method 35 | ++++ | 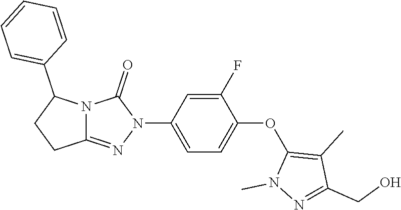 [SMILES: Cc1c(CO)nn(C)c1Oc1ccc(-n2nc3n(c2=O)C(c2ccccc2)CC3)cc1F] 2-(3-fluoro-4-((3-(hydroxymethyl)- 1,4-dimethyl-1H-pyrazol-5- yl)oxy)phenyl)-5-phenyl-2,5,6,7- tetrahydro-3H-pyrrolo[2,1-c] [1,2,4]triazol-3-one | white solid, yield: 3.07% | ¹H NMR (400 MHz, Chloroform-d) δ 7.89 (dd, J = 12.4, 2.4 Hz, 1H), 7.65 (d, J = 8.8 Hz, 1H), 7.37 (dq, J = 14.0, 7.2 Hz, 3H), 7.26-7.20 (m, 2H), 6.82 (t, J = 8.8 Hz, 1H), 5.28 (dd, J = 7.6, 4.0 Hz, 1H), 4.61 (s, 2H), 3.67 (s, 3H), 3.13-2.98 (m, 2H), 2.98-2.86 (m, 1H), 2.60-2.47 (m, 1H), 1.81 (s, 3H). | 436.2 |

### Example 159: Method 36

### 2-(4-((2-(aminomethyl)-4-methylthiazol-5-yl)oxy)-3-fluorophenyl)-4-(2,6-difluorobenzyl)-2,4-dihydro-3H-1,2,4-triazol-3-one

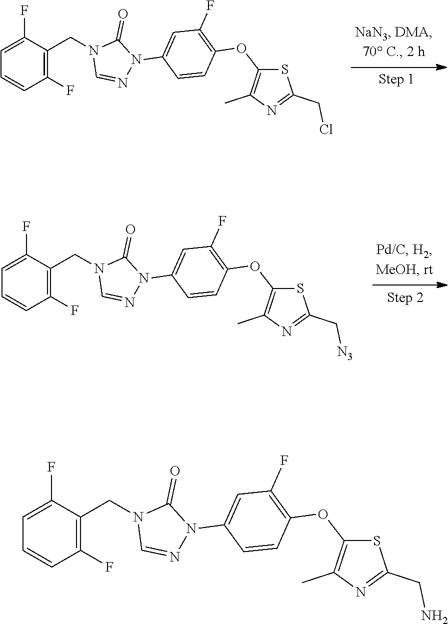 [SMILES: Cc1nc(CCl)sc1Oc1ccc(-n2ncn(Cc3c(F)cccc3F)c2=O)cc1F ; Cc1nc(CN=[N+]=[N-])sc1Oc1ccc(-n2ncn(Cc3c(F)cccc3F)c2=O)cc1F ; Cc1nc(CN)sc1Oc1ccc(-n2ncn(Cc3c(F)cccc3F)c2=O)cc1F]

### Step 1: 2-(4-((2-(azidomethyl)-4-methylthiazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(3-fluoro-4-((2-(hydroxymethyl)-4-methylthiazol-5-yl)oxy)phenyl)-2,4-dihydro-3H-1,2,4-triazol-3-one (100 mg, 0.22 mmol) and in DMA (10 mL) was added NaN3 (42.9 mg, 0.66 mmol). The resulted mixture was stirred for additional 2 h at 70° C. The filtrate was concentrated under reduced pressure to afford 2-(4-((2-(azidomethyl)-4-methylthiazol-5-yl)oxy)-3-fluorophenyl)-4-(2,6-difluorobenzyl)-2,4-d ihydro-3H-1,2,4-triazol-3-one (60 mg, 57.6% yield) as a white solid. LC-MS (m/z): 474.1[M+H]⁺.

### Step 2: 2-(4-((2-(aminomethyl)-4-methylthiazol-5-yl)oxy)-3-fluorophenyl)-4-(2,6-difluorobenzyl)-2,4-dihydro-3H-1,2,4-triazol-3-one

To a stirred solution of 2-(4-((2-(azidomethyl)-4-methylthiazol-5-yl)oxy)-3-fluorophenyl)-4-(2,6-difluorobenzyl)-2,4-dihydro-3H-1,2,4-triazol-3-one (60 mg, 0.12 mmol) in MeOH (10 mL) was added Pd/C (60 mg). The resulted mixture was stirred for additional 2 h at 25° C. under a nitrogen pressure of 1-2 atmosphere The resulted mixture was filtered and extracted with EtOAc (3×30 mL). The combined organic layers was concentrated under reduced pressure and purified by prep-HPLC to afford 2-(4-((2-(aminomethyl)-4-methylthiazol-5-yl)oxy)-3-fluorophenyl)-4-(2,6-difluorobenzyl)-2,4-dihydro-3H-1,2,4-triazol-3-one (30 mg, 55.9% yield) as a white solid. LC-MS (m/z): 448.1[M+H]⁺. ¹H NMR (400 MHz, CDCl₃) δ 7.82 (d, J=11.3 Hz, 1H), 7.65 (d, J=8.3 Hz, 1H), 7.59 (s, 1H), 7.35 (t, J=7.5 Hz, 1H), 6.97 (dd, J=14.5, 6.5 Hz, 3H), 4.96 (s, 2H), 4.36 (s, 2H), 2.20 (s, 3H).

Examples 160 was synthesized using a similar method to that used in Example 159.

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 159 Method 36 | ++++ | 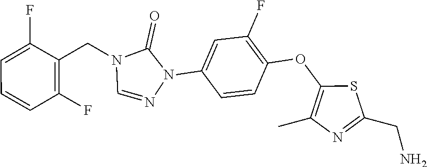 [SMILES: Cc1nc(CN)sc1Oc1ccc(-n2ncn(Cc3c(F)cccc3F)c2=O)cc1F] 2-(4-((2-(aminomethyl)- 4-methylthiazol- 5-yl)oxy)-3-fluorophenyl)- 4-(2,6-difluorobenzyl)-2,4- dihydro-3H-1,2,4-triazol-3-one | white solid, yield: 55.9% | ¹H NMR (400 MHz, CDCl₃) δ 7.82 (d, J = 11.3 Hz, 1H), 7.65 (d, J = 8.3 Hz, 1H), 7.59 (s, 1H), 7.35 (t, J = 7.5 Hz, 1H), 6.97 (dd, J = 14.5, 6.5 Hz, 3H), 4.96 (s, 2H), 4.36 (s, 2H), 2.20 (s, 3H). | 448.1 |
| Example 160 Method 36 | +++ | 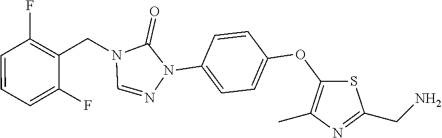 [SMILES: Cc1nc(CN)sc1Oc1ccc(-n2ncn(Cc3c(F)cccc3F)c2=O)cc1] 2-(4-((2-(aminomethyl)-4- methylthiazol-5-yl)oxy)phenyl)-4-(2, 6-difluorobenzyl)-2,4-dihydro- 3H-1,2,4-triazol-3-one | white solid, yield: 9.4% | ¹H NMR (400 MHz, DMSO-d₆) δ 8.42 (s, 1H), 7.92-7.83 (m, 2H), 7.48 (tt, J = 8.4, 6.8 Hz, 1H), 7.20- 7.10 (m, 4H), 4.97 (s, 2H), 4.32 (s, 2H), 2.23 (s, 3H). | 430.1 |

### Example 161: Method 37

### 4-(2,6-difluorobenzyl)-2-(3-phenoxycyclopentyl)-2,4-dihydro-3H-1,2,4-triazol-3-one

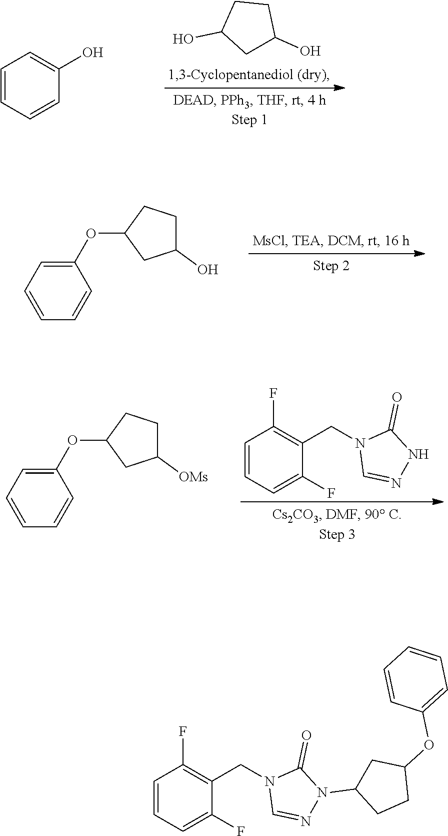 [SMILES: Oc1ccccc1 ; OC1CCC(O)C1 ; OC1CCC(Oc2ccccc2)C1 ; CS(=O)(=O)OC1CCC(Oc2ccccc2)C1 ; O=c1[nH]ncn1Cc1c(F)cccc1F ; O=c1n(Cc2c(F)cccc2F)cnn1C1CCC(Oc2ccccc2)C1]

### Step 1: 3-phenoxycyclopentan-1-ol

To a stirred solution of cyclopentane-1,3-diol (498 mg, 4.88 mmol), phenol (303 mg, 3.22 mmol) and PPh3 (1279 mg. 4.88 mmol) in THF (15 mL) under nitrogen at 0° C. was added a solution of DEAD (849 mg, 4.88 mol) in THF (5 mL) dropwise. The reaction mixture was stirred at 25° C. for 16 h. The reaction mixture was concentrated under pressure at 45° C. The residue was purified by silica gel column chromatography, eluted with (DCM: MeOH=20:1) to afford 3-phenoxycyclopentan-1-ol (468 mg, 53.9% yield) as a brown yellow oil. MS (m/z): 179.0 [M+H]⁺.

### Step 2: 3-phenoxycyclopentyl Methane Sulfonate

To a stirred solution of 3-phenoxycyclopentan-1-ol (450 mg, 2.52 mmol) and TEA (511 mg, 5.05 mmol) in DCM (10 mL) was MsCl (434 mg, 3.79 mmol) under nitrogen. The reaction mixture was stirred at 25° C. for 16 H. The reaction mixture was concentrated under pressure at 45° C. to afford 3-phenoxycyclopentyl methane sulfonate (400 mg, 61.2%) as a yellow solid. MS (m/z): 279.1 [M+Na]⁺.

### Step 3: 4-(2,6-difluorobenzyl)-2-(3-phenoxycyclopentyl)-2,4-dihydro-3H-1,2,4-triazol-3-one

To a stirred solution of 4-[(2,6-difluorophenyl) methyl]-2H-1,2,4-triazol-3-one (365 mg, 1.73 mmol) and 3-phenoxycyclopentyl methane sulfonate (443 mg, 1.73 mmol) in DMF (8 mL) was Cs2CO3 (1408 mg, 4.32 mmol) under nitrogen. The reaction mixture was stirred at 90° C. for 2h. The reaction mixture was concentrated under pressure at 50° C. The residue was extracted with EA (2×50 mL). The residue was purified by silica gel column chromatography, eluted with (PE:EtOAc) (1:1) to afford 4-(2,6-difluorobenzyl)-2-(3-phenoxycyclopentyl)-2,4-dihydro-3H-1,2,4-triazol-3-one (170 mg, 26.5% yield) as a white solid. LC-MS (m/z) 372.1 [M+H]⁺. ¹NMR (400 MHz, CDCl₃) δ 7.37 (s, 1H), 7.32 (ddd, J=13.0, 7.4, 4.2 Hz, 1H), 7.28-7.21 (m, 2H), 6.99-6.88 (m, 3H), 6.87-6.82 (m, 2H), 4.93 (ddd, J=13.3, 7.7, 5.1 Hz, 2H), 4.88 (s, 2H), 2.38-2.14 (m, 4H), 1.98-1.86 (m, 2H).

| Compound No. | | | Appearance | | |
|---|---|---|---|---|---|
| Method | Activity | Structure and Name | and Yield | ¹H NMR Data | MS(m/z) [M + H]⁺ |
| Example 161 Method 37 | ++ | 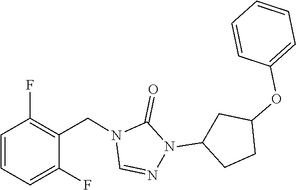 [SMILES: O=c1n(Cc2c(F)cccc2F)cnn1C1CCC(Oc2ccccc2)C1] 4-(2,6-difluorobenzyl)-2-(3- phenoxycyclopentyl)-2,4-dihydro- 3H-1,2,4-triazol-3-one | white solid, yield: 26.5% | ¹NMR (400 MHz, CDCl₃) δ 7.37 (s, 1H), 7.32 (ddd, J = 13.0, 7.4, 4.2 Hz, 1H), 7.28-7.21 (m, 2H), 6.99- 6.88 (m, 3H), 6.87-6.82 (m, 2H), 4.93 (ddd, J = 13.3, 7.7, 5.1 Hz, 2H), 4.88 (s, 2H), 2.38-2.14 (m, 4H), 1.98-1.86 (m, 2H). | 372.1 |

### Example 162: Method 38

### 4-(2,6-difluorobenzyl)-2-(4-((4-methylthiazol-5-yl)oxy)cyclohexyl)-2,4-dihydro-3H-1,2,4-tr iazol-3-one

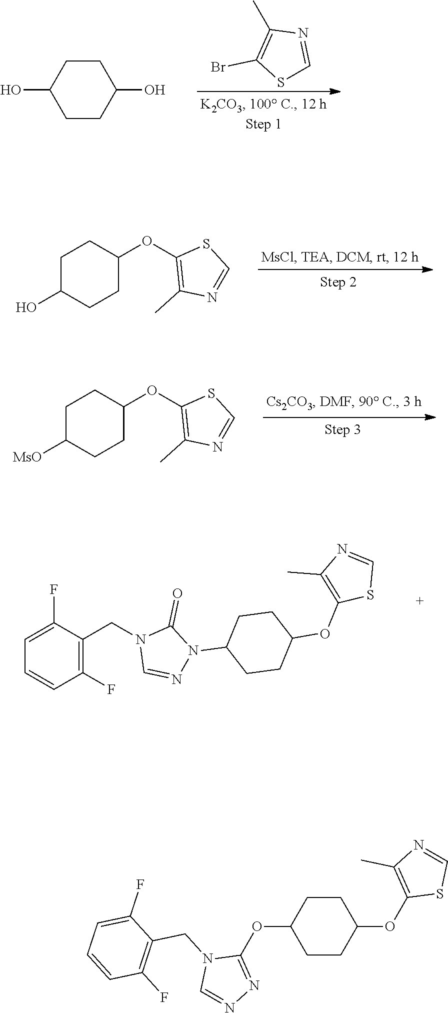

### Step 1: 4-((4-methylthiazol-5-yl)oxy)cyclohexan-1-ol

To a stirred solution of cyclohexane-1,4-diol (1 g, 8.6 mmol), 5-bromo-4-methylthiazole (1.5 g, 8.6 mmol) in DMF was K₂CO₃ (3.5 g, 25.8 mmol). The resulted mixture was stirred for additional 12 h at 100° C. The resulted mixture was diluted with water (50 mL). The resulted mixture was extracted with EtOAc (3×50 mL). The combined Organic layers were washed with brine (4 mL), dried over anhydrous Na₂SO₄, and after filtration, the filtrate was concentrated under reduced pressure. The residue was purified by flash chromatography (PE: EtOAc=1:1) to afford 4-((4-methylthiazol-5-yl)oxy)cyclohexan-1-ol (70 mg, 3.8% yield) as a yellow solid. LC-MS (m/z): 214.08 [M+H]⁺.

### Step 2: 4-((4-methylthiazol-5-yl)oxy)cyclohexyl methanesulfonate

To a stirred solution of 4-((4-methylthiazol-5-yl)oxy)cyclohexan-1-ol (45 mg, 0.21 mmol) and TEA (31.3 mg, 0.31 mmol) in DCM (5 mL) was added MsCl (23.9 mg, 0.31 mmol). The resulted mixture was stirred for additional 1 h at 25° C. The resulted mixture was diluted with water (10 mL). The resulted mixture was extracted with EtOAc (3×50 mL). The combined organic layers were washed with brine (5 mL), dried over anhydrous Na₂SO₄, and after filtration, the filtrate was concentrated under reduced pressure. The residue was purified by flash chromatography (PE:EtOAc=1:1) to afford 4-((4-methylthiazol-5-yl)oxy)cyclohexyl methanesulfonate (60 mg, 98.3% yield) as an oil. LC-MS (m/z): 292.06[M+H]⁺.

### Step 3: 4-(2,6-difluorobenzyl)-2-(4-((4-methylthiazol-5-yl)oxy)cyclohexyl)-2,4-dihydro-3H-1,2,4-triazol-3-one

To a stirred solution of 4-((4-methylthiazol-5-yl)oxy)cyclohexyl methanesulfonate (60 mg, 0.20 mmol) and 4-(2,6-difluorobenzyl)-2,4-dihydro-3H-1,2,4-triazol-3-one (42.2 mg, 0.20 mmol) in DMF (10 mL) was added Cs₂CO₃ (195.6 mg, 0.60 mmol). The resulted mixture was stirred for additional 3 h at 90° C. The resulted mixture was diluted with water (10 mL). The resulted mixture was extracted with EtOAc (3×30 mL). The combined organic layers were washed with brine (5 mL), dried over anhydrous Na₂SO₄, and after filtration, the filtrate was concentrated under reduced pressure. The residue was purified by flash chromatography with the following conditions: column: Gemini 5u C₁₈ 150×21.2 mm; Mobile phase B: ACN; Flow rate: 20 mL/min; Gradient: 40% B-60% B in 20 min; Detector: 214 nm. The desired fractions were concentrated under reduced pressure to afford 4-(2,6-difluorobenzyl)-2-(4-((4-methylthiazol-5-yl)oxy)cyclohexyl)-2,4-dihydro-3H-1,2,4-triaz ol-3-one (12 mg, 14.7% yield) as a white solid. LC-MS (m/z): 407.1 [M+H]⁺. ¹H NMR (400 MHz, CDCl₃) δ 8.68 (s, 1H), 7.40 (s, 1H), 7.35 (td, J=8.4, 4.2 Hz, 1H), 6.97 (dd, J=13.4, 5.8 Hz, 2H), 4.92 (s, 2H), 4.17 (m, 1H), 3.97 (m, 1H), 2.35 (s, 3H) 2.28 (d, J=11.1 Hz, 2H), 2.01 (d, J=10.6 Hz, 2H), 1.86 (td, J=13.3, 3.1 Hz, 2H), 1.69 (m, 2H).

| Compound No. | | | Appearance | | |
|---|---|---|---|---|---|
| Method | Activity | Structure and Name | and Yield | ¹H NMR Data | MS(m/z) [M + H]⁺ |
| Example 162 Method 38 | +++ | 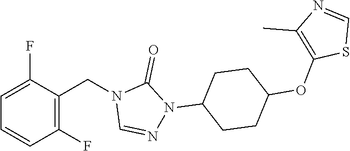 [SMILES: Cc1ncsc1OC1CCC(n2ncn(Cc3c(F)cccc3F)c2=O)CC1] 4-(2,6-difluorobenzyl)-2-(4-((4- methylthiazol-5-yl)oxy)cyclohexyl)- 2,4-dihydro-3H-1,2,4-triazol- 3-one | white solid, yield: 14.7% | ¹H NMR (400 MHz, CDCl₃) δ 8.68 (s, 1H), 7.40 (s, 1H), 7.35 (td, J = 8.4, 4.2 Hz, 1H), 6.97 (dd, J = 13.4, 5.8 Hz, 2H), 4.92 (s, 2H), 4.17 (m, 1H), 3.97 (m, 1H), 2.35 (s, 3H) 2.28 (d, J = 11.1 Hz, 2H), 2.01 (d, J = 10.6 Hz, 2H), 1.86 (td, J = 13.3, 3.1 Hz, 2H), 1.69 (m, 2H). | 407.1 |

### Example 163: Method 39

### 2-(4-((2-(1-aminoethyl)-4-methylthiazol-5-yl)oxy)-3-fluorophenyl)-4-(2,6-difluorobenzyl)-2,4-dihydro-3H-1,2,4-triazol-3-one

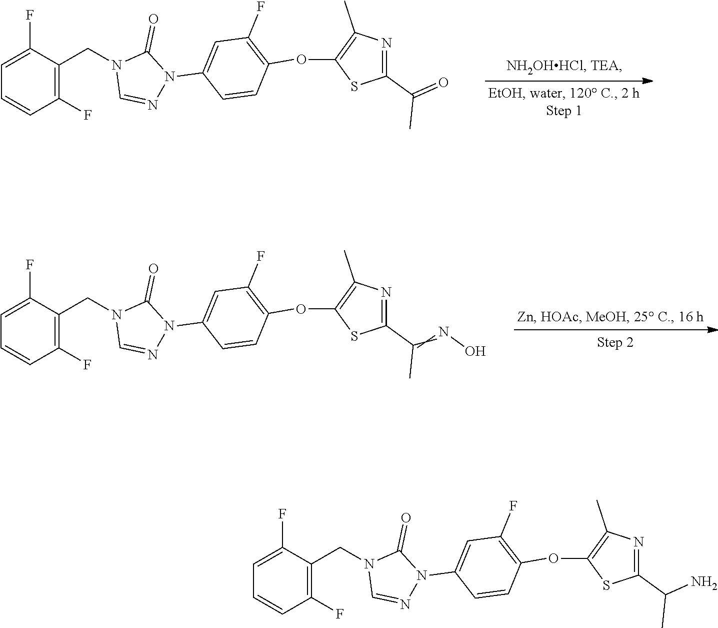 [SMILES: CC(=O)c1nc(C)c(Oc2ccc(-n3ncn(Cc4c(F)cccc4F)c3=O)cc2F)s1 ; CC(=NO)c1nc(C)c(Oc2ccc(-n3ncn(Cc4c(F)cccc4F)c3=O)cc2F)s1 ; Cc1nc(C(C)N)sc1Oc1ccc(-n2ncn(Cc3c(F)cccc3F)c2=O)cc1F]

### Step 1

### 4-(2,6-difluorobenzyl)-2-(3-fluoro-4-((2-(1-(hydroxyimino)ethyl)-4-methylthiazol-5-yl)oxy)phenyl)-2,4-dihydro-3H-1,2,4-triazol-3-one

A solution of 2-(4-((2-acetyl-4-methylthiazol-5-yl)oxy)-3-fluorophenyl)-4-(2,6-difluorobenzyl)-2,4-dihydro-3H-1,2,4-triazol-3-one (50 mg, 0.1 mmol), NH₂OH·HCl (15 mg, 0.2 mmol), and TEA (33 mg, 0.3 mmol) in DMF (2 mL) was stirred at 120° C. for 2 h. The resulted solution was diluted with EtOAc (20 mL), washed with brine (20 mL), dried over Na₂SO₄ and concentrated to give a white solid. It was used for next step without further purification. MS (m/z): 476.0 [M+H]⁺. ¹H NMR (400 MHz, DMSO-d₆) δ 11.79 (s, 1H), 8.34 (s, 1H), 7.88 (dd, J=12.7, 2.4 Hz, 1H), 7.68 (d, J=9.1 Hz, 1H), 7.47 (d, J=8.2 Hz, 1H), 7.30 (t, J=9.1 Hz, 1H), 7.16 (t, J=8.1 Hz, 2H), 4.98 (s, 2H), 2.24 (s, 3H), 2.16 (s, 3H).

### Step 2: 2-(4-((2-(1-aminoethyl)-4-methylthiazol-5-yl)oxy)-3-fluorophenyl)-4-(2,6-difluorobenzyl)-2,4-dihydro-3H-1,2,4-triazol-3-one

The above white solid was diluted with MeOH (5 mL), Zn (14 mg, 0.2 mmol) and NH₄Cl (12 mg, 0.2 mmol) was added and stirred at rt for 16 h. LCMS showed 60% of product conversion and 40% of S.M. (starting material) remained. The resulted mixture was diluted with water (30 mL). The resulted mixture was extracted with EtOAc (3×20 mL). The combined organic layers were washed with brine (30 mL), dried over anhydrous Na₂SO₄, and after filtration, the filtrate was concentrated under reduced pressure. The residue was purified by prep-HPLC to afford 2-(4-((2-(1-aminoethyl)-4-methylthiazol-5-yl)oxy)-3-fluorophenyl)-4-(2,6-difluorobenzyl)-2,4-dihydro-3H-1,2,4-triazol-3-one (13.2 mg, 26.4% yield) as a white solid.

MS (m/z): 462.0 [M+H]⁺. ¹H NMR (400 MHz, CDCl₃) δ 7.88 (d, J=12.0 Hz, 1H), 7.70 (d, J=8.9 Hz, 1H), 7.57 (s, 1H), 7.43-7.31 (m, 1H), 6.99 (q, J=8.1 Hz, 3H), 4.97 (s, 2H), 4.35 (s, 1H), 3.73 (s, 2H), 2.26 (s, 3H), 1.58 (t, J=11.0 Hz, 3H).

Example (Compound) 164 was synthesized using a similar method to that used in Example 163.

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 163 Method 39 | ++++ | 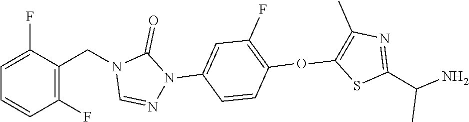 [SMILES: Cc1nc(C(C)N)sc1Oc1ccc(-n2ncn(Cc3c(F)cccc3F)c2=O)cc1F] 2-(4-((2-(1-aminoethyl)-4- methylthiazol-5-yl)oxy)-3-fluorophenyl)- 4-(2,6-difluorobenzyl)-2,4- dihydro-3H-1,2,4-triazol-3-one | white solid, yield: 26.4% | ¹H NMR (400 MHz, CDCl₃) δ 7.88 (d, J = 12.0 Hz, 1H), 7.70 (d, J = 8.9 Hz, 1H), 7.57 (s, 1H), 7.43-7.31 (m, 1H), 6.99 (q, J = 8.1 Hz, 3H), 4.97 (s, 2H), 4.35 (s, 1H), 3.73 (s, 2H), 2.26 (s, 3H), 1.58 (t, J = 11.0 Hz, 3H). | 462.0 |
| Example 164 Method 39 | ++++ | 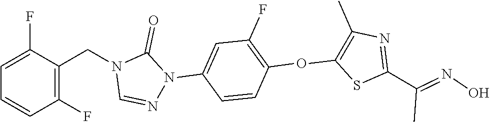 [SMILES: C/C(=N\O)c1nc(C)c(Oc2ccc(-n3ncn(Cc4c(F)cccc4F)c3=O)cc2F)s1] 4-(2,6-difluorobenzyl)-2-(3-fluoro- 4-((2-(1-(hydroxyimino)ethyl)- 4-methylthiazol-5-yl)oxy)phenyl)- 2,4-dihydro-3H-1,2,4-triazol- 3-one | white solid, yield: 18.4% | ¹H NMR (400 MHz, DMSO-d₆) δ 11.79 (s, 1H), 8.34 (s, 1H), 7.88 (dd, J = 12.7, 2.4 Hz, 1H), 7.68 (d, J = 9.1 Hz, 1H), 7.47 (d, J = 8.2 Hz, 1H), 7.30 (t, J = 9.1 Hz, 1H), 7.16 (t, J = 8.1 Hz, 2H), 4.98 (s, 2H), 2.24 (s, 3H), 2.16 (s, 3H) | 476.0 |

### Example 165: Method 40

### 4-(2,6-difluorobenzyl)-2-(2-phenoxythiazol-5-yl)-2,4-dihydro-3H-1,2,4-triazol-3-one

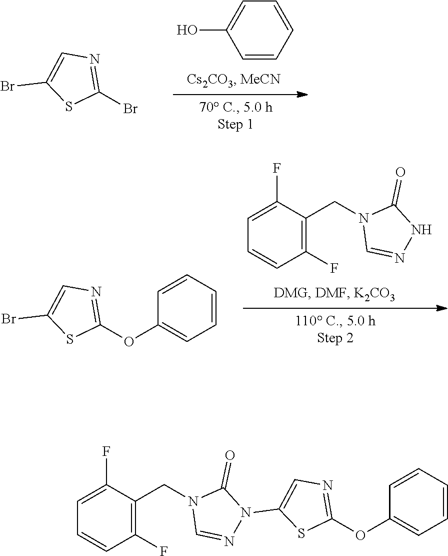 [SMILES: Brc1cnc(Br)s1 ; Oc1ccccc1 ; O=c1[nH]ncn1Cc1c(F)cccc1F ; Brc1cnc(Oc2ccccc2)s1 ; O=c1n(Cc2c(F)cccc2F)cnn1-c1cnc(Oc2ccccc2)s1]

### Step 1: 5-bromo-2-phenoxythiazole

To a stirred solution of 2,5-dibromothiazole (500 mg, 2.06 mmol) and phenol (194 mg 2.06 mmol) in ACN (8 mL) was added Cs₂CO₃ (2012 mg 6.17 mmol) at rt. The resulted mixture was stirred for additional 5 h at 70° C. The mixture was allowed to cool down to rt. The resulted mixture was diluted with water (50 mL). The resulted mixture was extracted with EtOAc (3×30 mL). The combined organic layers were washed with brine (50 mL), dried over anhydrous Na₂SO₄, and after filtration, the filtrate was concentrated under reduced pressure. The residue was purified by silica gel column chromatography, eluted with (PE:EtOAc=1:1) to afford 5-bromo-2-phenoxythiazole (350 mg, 66.4% yield) as a white solid. MS (m/z): 255.9, 257.9 [M+H]⁺.

### Step 2: 4-(2,6-difluorobenzyl)-2-(2-phenoxythiazol-5-yl)-2,4-dihydro-3H-1,2,4-triazol-3-one

To a stirred solution of 5-bromo-2-phenoxythiazole (150 mg, 0.59 mmol) and 4-(2,6-difluorobenzyl)-2,4-dihydro-3H-1,2,4-triazol-3-one (124 mg, 0.59 mmol) in DMF (5 mL) were added CuI (56 mg, 0.29 mmol), DMG (30 mg, 0.29 mmol), and K₂CO₃ (243 mg, 1.76 mmol) at rt. The resulted mixture was stirred for additional 5 h at 110° C. under Ar. The mixture was allowed to cool down to rt. The resulted mixture was diluted with water (30 mL). The resulted mixture was extracted with EtOAc (3×15 mL). The combined organic layers were washed with brine (25 mL), dried over anhydrous Na₂SO₄, and after filtration, the filtrate was concentrated under reduced pressure. The residue was purified by reverse phase flash chromatography with the following conditions: column: Spherical C₁₈ 40-60 um, 40 g; Mobile phase B: ACN; Flow rate: 40 mL/min; Gradient: 35% B-60% B in 20 min; Detector: 254 nm. The fractions containing the desired product were collected at 51% B and concentrated under reduced pressure to afford the titled compound (20 mg, 8.840 yield) as an off-white solid. LC-MS (m/z): 387.0 [M+H]⁺. ¹H NMR (400 MHz, DMSO-d₆) δ 8.37 (s, 1H), 7.52-7.42 (m, 3H), 7.41-7.24 (m, 4H), 7.15 (t, J=8.1 Hz, 2H), 4.96 (s, 2H).

Example (Compound) 166 was synthesized using a similar method to that used in Example 165.

| Com- | | | | | |
|---|---|---|---|---|---|
| pound No. | | | Appearance | | |
| Method | Activity | Structure and Name | and Yield | ¹H NMR Data | MS(m/z) [M + H]⁺ |
| Example 165 Method 40 | +++ | 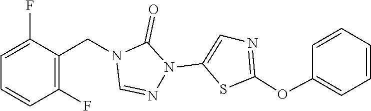 [SMILES: O=c1n(Cc2c(F)cccc2F)cnn1-c1cnc(Oc2ccccc2)s1] 4-(2,6-difluorobenzyl)-2-(2- phenoxythiazol-5-yl)-2,4-dihydro- 3H-1,2,4-triazol-3-one | white solid, yield: 8.84% | ¹H NMR (400 MHz, DMSO-d₆) δ 8.37 (s, 1H), 7.52-7.42 (m, 3H), 7.41-7.24 (m, 4H), 7.15 (t, J = 8.1 Hz, 2H), 4.96 (s, 2H). | 387.0 |
| Example 166 Method 40 | +++ | 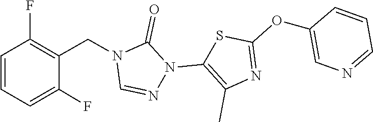 [SMILES: Cc1nc(Oc2cccnc2)sc1-n1ncn(Cc2c(F)cccc2F)c1=O] 4-(2,6-difluorobenzyl)-2-(4-methyl- 2-(pyridin-3-yloxy)thiazol-5- yl)-2,4-dihydro-3H-1,2,4-triazol- 3-one | white solid, yield: 29.2% | ¹H NMR (400 MHz, DMSO-d₆) δ 8.68 (d, J = 2.8 Hz, 1H), 8.54 (dd, J = 4.8, 1.2 Hz, 1H), 8.28 (s, 1H), 7.95-7.88 (m, 1H), 7.58-7.42 (m, 2H), 7.16 (t, J = 8.0 Hz, 2H), 4.94 (s, 2H), 2.09 (s, 3H). | 402.0 |

### Example 167: Method 41

### 3-(5-(4-(4-(2,6-difluorobenzyl)-5-oxo-4,5-dihydro-1H-1,2,4-triazol-1-yl)-2-fluorophenoxy)-4-methylthiazol-2-yl)cyclobutane-1-carboxamide

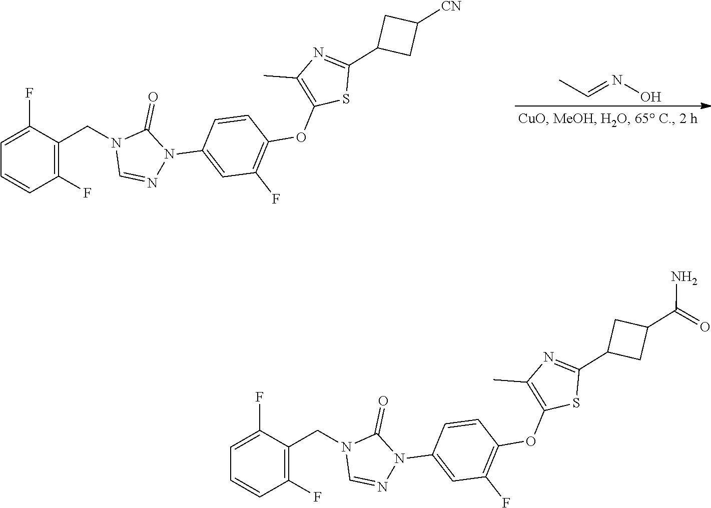

To a stirred solution of 3-(5-(4-(4-(2,6-difluorobenzyl)-5-oxo-4,5-dihydro-1H-1,2,4-triazol-1-yl)-2-fluorophenoxy)-4-methylthiazol-2-yl)cyclobutane-1-carbonitrile (2.4 mg, 4.8 mmol) and (E)-acetaldehyde oxime (0.28 mg, 4.8 mmol) in MeOH:H2O (2 mL) was added CuO(0.75 mg, 9.6 mmol). The resulted mixture was stirred for additional 2 h at 65° C. The resulted mixture was diluted with water (2 mL). The resulted mixture was extracted with EtOAc (3×5 mL). The combined organic layers were washed with brine (4 mL), dried over anhydrous Na₂SO₄, and after filtration, the filtrate was concentrated under reduced pressure. The residue was purified by flash chromatography with the following conditions: column: Gemini 5u C₁₈ 150×21.2 mm; Mobile phase B: ACN; Flow rate: 20 mL/min; Gradient: 40%₀B-60%₀B in 20 min; Detector: 214 nm. The desired fractions were concentrated under reduced pressure to afford the titled compound (1.02 mg, 41.1% yield) as an off-white solid. LC-MS (m/z): 516.1 [M+H]⁺. ¹H NMR (400 MHz, CDCl₃) δ 7.90-7.92 (d, J=10.6 Hz, 1H), 7.73 (d, J=7.3 Hz, 1H), 7.58 (s, 1H), 7.32-7.38 (m, 1H), 6.96-7.04 (m, 3H), 6.12 (br, 1H), 5.59 (br, 1H), 4.96 (s, 2H), 3.92 (s, 1H), 3.22 (s, 1H), 2.78 (br, 2H), 2.68 (br, 2H), 2.34 (s, 3H).

Example (Compound) 168 was synthesized using a similar method to that used in Example 167.

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 167 Method 41 | ++++ | 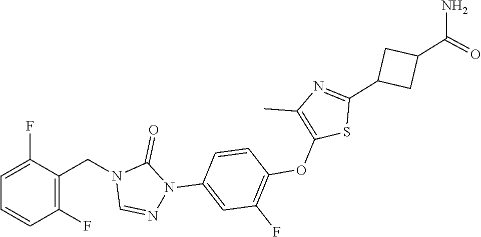 [SMILES: Cc1nc(C2CC(C(N)=O)C2)sc1Oc1ccc(-n2ncn(Cc3c(F)cccc3F)c2=O)cc1F] 3-(5-(4-(4-(2,6-difluorobenzyl)- 5-oxo-4,5-dihydro-1H-1,2,4-triazol- 1-yl)-2-fluorophenoxy)-4- methylthiazol-2-yl)cyclobutane-1- carboxamide | white solid, yield: 41.1% | ¹H NMR (400 MHz, CDCl₃) δ 7.90-7.92 (d, J = 10.6 Hz, 1H), 7.73 (d, J = 7.3 Hz, 1H), 7.58 (s, 1H), 7.32-7.38 (m, 1H), 6.96- 7.04 (m, 3H), 6.12 (br, 1H), 5.59 (br, 1H), 4.96 (s, 2H), 3.92 (s, 1H), 3.22 (s, 1H), 2.78 (br, 2H), 2.68 (br, 2H), 2.34 (s, 3H). | 516.1 |
| Example 168 Method 41 | ++++ | 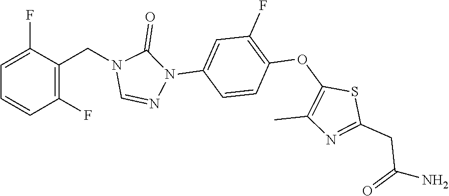 [SMILES: Cc1nc(CC(N)=O)sc1Oc1ccc(-n2ncn(Cc3c(F)cccc3F)c2=O)cc1F] 2-(5-(4-(4-(2,6-difluorobenzyl)- 5-oxo-4,5-dihydro-1H-1,2,4- triazol-1-yl)-2-fluorophenoxy)-4- methylthiazol-2-yl)acetamide | white solid, yield: 34.6% | ¹H NMR (400 MHz, DMSO-d₆) δ 8.35 (s, 1H), 7.87 (dd, J = 12.6, 2.5 Hz, 1H), 7.72- 7.63 (m, 2H), 7.48 (s, 1H), 7.19 (dt, J = 16.2, 8.6 Hz, 4H), 4.97 (s, 2H), 3.75 (s, 2H), 2.18 (s, 3H). | 476.1 |

### Example 169: Method 42

### 4-(2,6-difluorobenzyl)-2-(2-((2-methoxypyridin-4-yl)oxy)-4-methylthiazol-5-yl)-2,4-dihydr o-3H-1,2,4-triazol-3-one

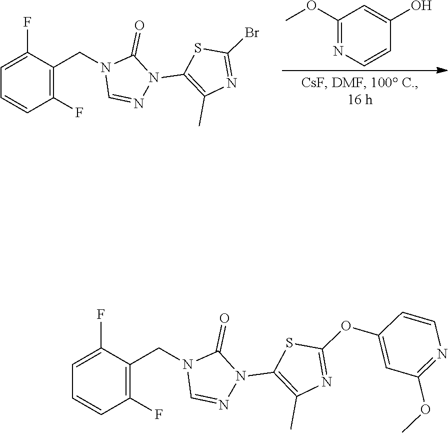

A solution of 2-(2-bromo-4-methylthiazol-5-yl)-4-(2,6-difluorobenzyl)-2,4-dihydro-3H-1,2,4-triazol-3-one (500 mg, 1.3 mmol), 2-methoxypyridin-4-ol (162 mg, 1.3 mmol) and CsF (392 mg, 2.6 mmol) in DMF (10 mL) was stirred for 16 h at 100° C. The mixture was allowed to cool down to rt. The resulted mixture was diluted with water (30 mL). The resulted mixture was extracted with EtOAc (3×20 mL). The combined organic layers were washed with brine (30 mL), dried over anhydrous Na₂SO₄. After filtration, the filtrate was concentrated under reduce pressure. The residue was purified by Combi-Flash (PE:EtOAc) (1:1) to afford 4-(2,6-difluorobenzyl)-2-(2-((2-methoxypyridin-4-yl)oxy)-4-methylthiazol-5-yl)-2,4-dihydro-3H-1,2,4-triazol-3-one (270 mg, 43.5% yield) as a brown solid. MS (m/z): 432.0 [M+H]⁺. ¹H NMR (400 MHz, CDCl₃) δ 8.21 (s, 1H), 7.57 (s, 1H), 7.44-7.34 (m, 1H), 6.99 (dd, J=13.6, 5.7 Hz, 2H), 6.87 (d, J=4.8 Hz, 1H), 6.73 (s, 1H), 4.97 (s, 2H), 3.97 (s, 3H), 2.29 (s, 3H).

| Compound No. | | | Appearance | | MS(m/z) |
|---|---|---|---|---|---|
| Method | Activity | Structure and Name | and Yield | ¹H NMR Data | [M + H]⁺ |
| Example 169 Method 42 | ++ | 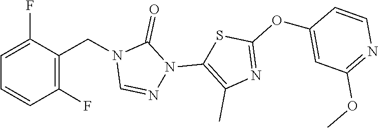 [SMILES: COc1cc(Oc2nc(C)c(-n3ncn(Cc4c(F)cccc4F)c3=O)s2)ccn1] 4-(2,6-difluorobenzyl)-2-(2-((2- methoxypyridin-4-yl)oxy)-4- methylthiazol-5-yl)-2,4-dihydro- 3H-1,2,4-triazol-3-one | white solid, yield: 43.5% | ¹H NMR (400 MHz, CDCl₃) δ 8.21 (s, 1H), 7.57 (s, 1H), 7.44- 7.34 (m, 1H), 6.99 (dd, J = 13.6, 5.7 Hz, 2H), 6.87 (d, J = 4.8 Hz, 1H), 6.73 (s, 1H), 4.97 (s, 2H), 3.97 (s, 3H), 2.29 (s, 3H). | 432.0 |

### Example 170: Method 43

### 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetic acid

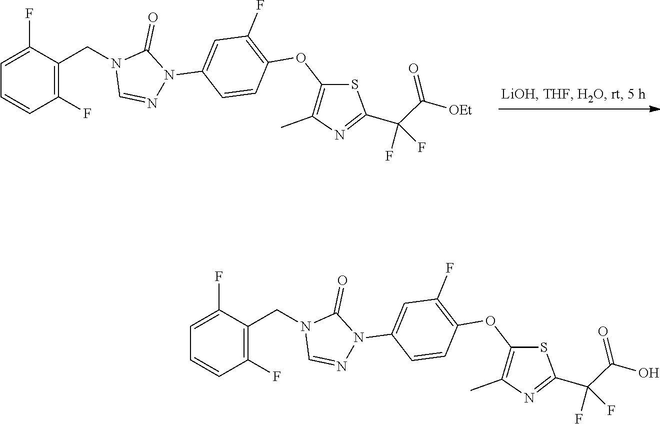 [SMILES: CCOC(=O)C(F)(F)c1nc(C)c(Oc2ccc(-n3ncn(Cc4c(F)cccc4F)c3=O)cc2F)s1 ; Cc1nc(C(F)(F)C(=O)O)sc1Oc1ccc(-n2ncn(Cc3c(F)cccc3F)c2=O)cc1F]

To a stirred solution of ethyl 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetate (30 mg, 0.06 mmol) in THF/H2O mixture solvent (3/1, 4 mL) was added LiOH. The reaction solution was stirred at rt for 5 h. The resulted solution was adjusted to pH 6 and concentrated under reduced pressure. The residue was purified by TLC. (DCM/MeOH) (10/1) to afford 2-(5-(4-(4-(2,6-difluorobenzyl)-5-oxo-4,5-dihydro-1H-1,2,4-triazol-1-yl)-2-fluorophenoxy)-4-methylthiazol-2-yl)-2,2-difluoroacetic acid (13 mg, 42.3% yield) as a white solid. MS (m/z): 513.2 [M+H]⁺. ¹H NMR (400 MHz, MeOD) δ 7.90 (s, 1H), 7.80 (dd, J=12.4, 2.4 Hz, 1H), 7.64 (d, J=9.2 Hz, 1H), 7.41-7.29 (m, 1H), 7.12 (t, J=9.2 Hz, 1H), 6.99-6.92 (m, 2H), 4.93 (s, 2H), 2.20 (s, 3H).

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 170 Method 43 | +++ | 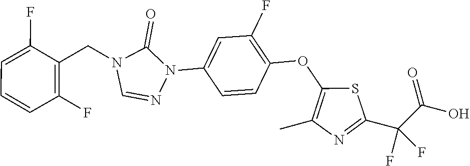 [SMILES: Cc1nc(C(F)(F)C(=O)O)sc1Oc1ccc(-n2ncn(Cc3c(F)cccc3F)c2=O)cc1F] 2-(5-(4-(4-(2,6-difluorobenzyl)- 5-oxo-4,5-dihydro-1H-1,2,4-triazol- 1-yl)-2-fluorophenoxy)-4- methylthiazol-2-yl)-2,2- difluoroacetic acid | white solid, yield: 42.3% | ¹H NMR (400 MHz, MeOD) δ 7.90 (s, 1H), 7.80 (dd, J = 12.4, 2.4 Hz, 1H), 7.64 (d, J = 9.2 Hz, 1H), 7.41-7.29 (m, 1H), 7.12 (t, J = 9.2 Hz, 1H), 6.99-6.92 (m, 2H), 4.93 (s, 2H), 2.20 (s, 3H). | 513.2 |

### Example 171: Method 44

methyl

### 5-((4-(4-(2,6-difluorobenzyl)-5-oxo-4,5-dihydro-1H-1,2,4-triazol-1-yl)phenyl)amino)thiazole-4-carboxylate

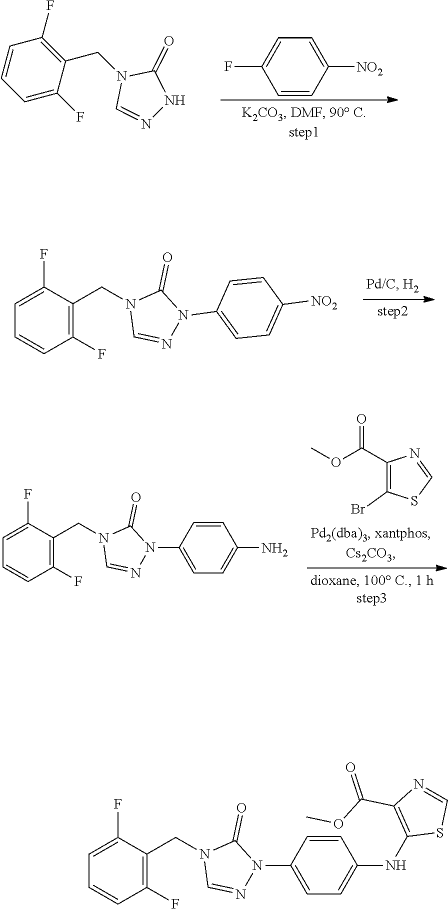

### Step 1: Preparation of 4-(2,6-difluorobenzyl)-2-(4-nitrophenyl)-2,4-dihydro-3H-1,2,4-triazol-3-one

To a stirred solution of 4-(2,6-difluorobenzyl)-2,4-dihydro-3H-1,2,4-triazol-3-one (0.5 g, 2.37 mmol) and 1-fluoro-4-nitrobenzene (334 mg, 2.37 mmol) in DMF (20 mL) were added K₂CO₃(654 mg, 4.74 mmol) at rt. The resulted mixture was stirred for additional 2 h at 90° C. The mixture was allowed to cool down to rt. The resulted mixture was diluted with water (50 mL). The resulted mixture was extracted with EtOAc (3×30 mL). The combined organic layers were washed with brine (20 mL), dried over anhydrous Na₂SO₄, and after filtration, the filtrate was concentrated under reduced pressure. The residue was purified by silica gel column chromatography, eluted with (PE:EtOAc) (1:1) to afford 4-(2,6-difluorobenzyl)-2-(4-nitrophenyl)-2,4-dihydro-3H-1,2,4-triazol-3-one (530 mg, 67.4% yield) as a brown yellow solid. LCMS (m/z): 333.0 [M+H]+, RT: 1.303 min.

### Step 2: Preparation 2-(4-aminophenyl)-4-(2,6-difluorobenzyl)-2,4-dihydro-3H-1,2,4-triazol-3-one

Pd/C (250 mg) was added to a solution of 4-[(2,6-difluorophenyl) methyl]-2-(4-nitrophenyl)-1,2,4-triazol-3-one(510 mg, 1.53 mmol) in MeOH (10 mL), the resulted mixture was stirred at rt under H₂ for 2h, and then filtrated to give the desired product as a yellow solid (450 mg, 97% yield). LCMS (m/z): 303.1 [M+H]⁺, RT: 1.015 min.

### Step 3: Preparation of methyl 5-((4-(4-(2,6-difluorobenzyl)-5-oxo-4,5-dihydro-1H-1,2,4-triazol-1-yl)phenyl)amino)thiazole-4-carboxylate

A mixture solution of 2-(4-aminophenyl)-4-(2,6-difluorobenzyl)-2,4-dihydro-3H-1,2,4-triazol-3-one (170 mg, 0.56 mmol), methyl 5-bromothiazole-4-carboxylate (150 mg, 0.67 mmol), Cs₂CO₃ (366 mg, 1.12 mmol), xantphos (65 mg, 0.11 mmol), Pd₂(dba)₃(103 mg, 0.11 mmol), and dioxane (10 mL) was stirred at 110° C. for 1 h. Then the mixture was cooled to room temperature, diluted with water (30 mL), and extracted three times with ethyl acetate (20 mL). The organic layers were combined, the solvent was removed under vacuum and the crude was purified by Prep-HPLC (column-Gemini —C₁₈ 150×21.2 mm, Sum; Mobile phase: ACN-H₂O (0.1% FA), 5%-20%) to afford the desired product as a white solid (20 mg, 8.0% yield). LCMS (m/z): 444.0 [M+H]⁺, RT: 1.246 min. ¹H NMR (400 MHz, CDCl₃) δ 9.71 (s, 1H), 8.01-7.98 (m, 3H), 7.58 (s, 1H), 7.37 (s, 1H), 7.30 (d, J=8.9 Hz, 1H), 6.99 (m, 2H), 4.98 (s, 2H), 3.98 (s, 3H).

| Compound No. | | | Appearance | | |
|---|---|---|---|---|---|
| Method | Activity | Structure and Name | and Yield | ¹H NMR Data | MS(m/z) [M + H]⁺ |
| Example 171 Method 44 | ++ | 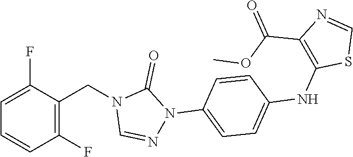 [SMILES: COC(=O)c1ncsc1Nc1ccc(-n2ncn(Cc3c(F)cccc3F)c2=O)cc1] methyl 5-((4-(4-(2,6-difluorobenzyl)-5- oxo-4,5-dihydro-1H-1,2,4-triazol- 1-yl)phenyl)amino)thiazole-4- carboxylate | white solid, yield: 8.0% | ¹H NMR (400 MHz, CDCl₃) δ 9.71 (s, 1H), 8.01-7.98 (m, 3H), 7.58 (s, 1H), 7.37 (s, 1H), 7.30 (d, J = 8.9 Hz, 1H), 6.99 (m, 2H), 4.98 (s, 2H), 3.98 (s, 3H). | 444.0 |

### Example 172: Method 45

### 4-(2,6-difluorobenzyl)-2-(4-((1,3-dimethyl-1H-pyrazol-5-yl)oxy)-3-fluorophenyl)-2,4-dihy dro-3H-1,2,4-triazol-3-one

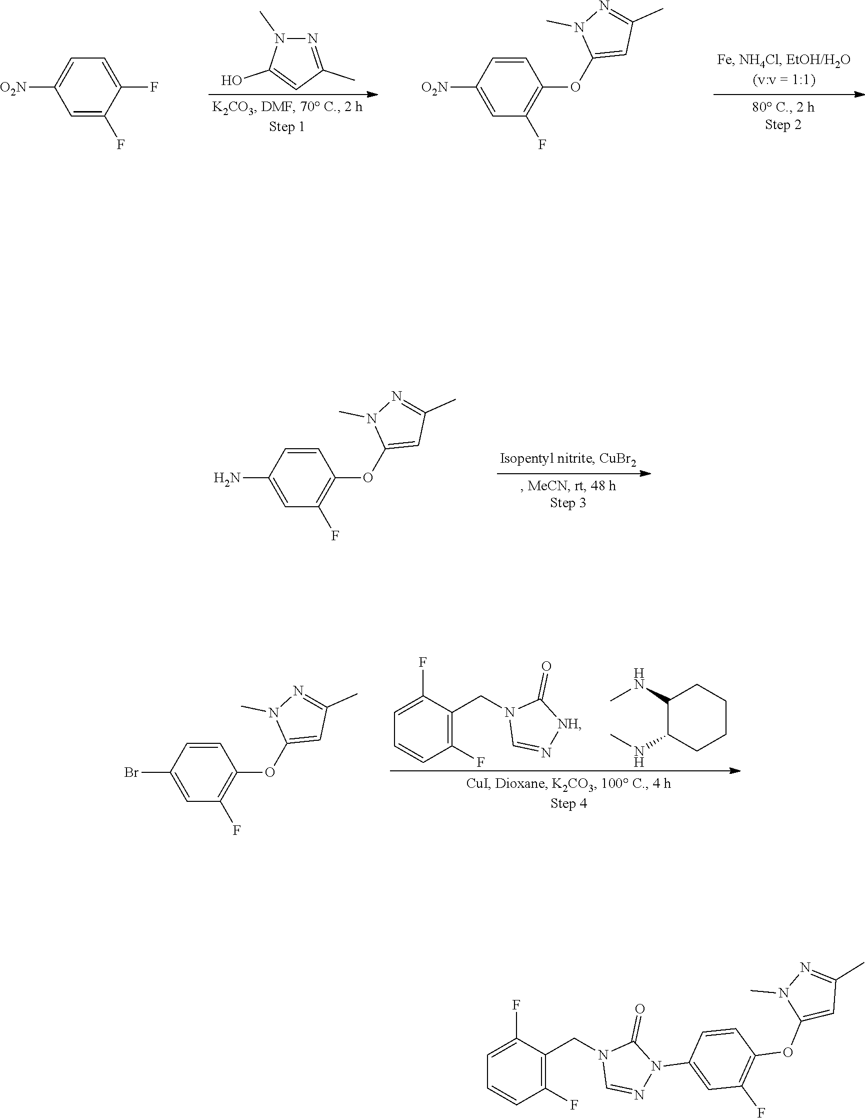

### Step 1: 5-(2-fluoro-4-nitrophenoxy)-1,3-dimethyl-1H-pyrazole

To a stirred solution of 1,2-difluoro-4-nitrobenzene (6.4 g, 0.04 mol) and 2,5-dimethylpyrazol-3-ol (4.51 g, 0.04 mol) in DMF (35 mL) were added K₂CO₃ (16.8 g, 0.08 mol) at rt. The resulted mixture was stirred for additional 2 h at 70° C. under N₂. The mixture was allowed to cool down to rt. The resulted mixture was diluted with water (30 mL). The resulted mixture was extracted with EtOAc (3×30 mL). The combined organic layers were washed with brine (100 mL), dried over anhydrous Na₂SO₄, and after filtration, the filtrate was concentrated under reduced pressure. The residue was purified by silica gel column chromatography, eluted with EA/PE=¹-15% to afford 5-(2-fluoro-4-nitrophenoxy)-1,3-dimethyl-1H-pyrazole (8.4 g, 89% yield) as a yellow solid. MS (m/z): 252.0 [M+H]⁺.

### Step 2: 4-((1,3-dimethyl-1H-pyrazol-5-yl)oxy)-3-fluoroaniline

To a stirred solution of 5-(2-fluoro-4-nitrophenoxy)-1,3-dimethylpyrazole (2.5 g, 0.01 mol) and Fe (5.5 g, 0.1 mol) in EtOH/H₂O (80 mL) were added NH₄Cl (5.3 g, 0.1 mol) at rt. The resulted mixture was stirred for additional 2 h at 80° C. The mixture was allowed to cool down to rt. The resulted mixture was diluted with water (80 mL). The resulted mixture was extracted with EtOAc (3×40 mL). The combined organic layers were washed with brine (80 mL), dried over anhydrous Na₂SO₄, and after filtration, the filtrate was concentrated under reduced pressure. The residue was purified by silica gel column chromatography, eluted with (PE:EtOAc=1-20%) to afford 4-((1,3-dimethyl-1H-pyrazol-5-yl)oxy)-3-fluoroaniline (1.2 g, 68.1% yield) as a yellow solid. MS (m/z): 222.2 [M+H]⁺.

### Step 3: 5-(4-bromo-2-fluorophenoxy)-1,3-dimethyl-1H-pyrazole

To a stirred solution of 4-[(2,5-dimethylpyrazol-3-yl)oxy]-3-fluoroaniline (1.9 g, 8.6 mmol) in MeCN (20 mL) was added isopentyl nitrite (1.2 g, 9.46 mmol) dropwise at 0° C. under N₂. The resulted mixture was stirred at 0° C. for 10 min. Then CuBr₂ (1.97 g, 9.46 mmol) was added into above reaction and stirred at r.t for 48 hrs. The resulted reaction mixture was filtered and the residue was extracted with EtOAc (3×45 mL). The combined organic layers were washed with brine (30 mL), dried over anhydrous Na₂SO₄, and after filtration, the filtrate was concentrated under reduced pressure. The residue was purified by silica gel column chromatography, eluted with (PE:EtOAc) (1:2) to afford 5-(4-bromo-2-fluorophenoxy)-1,3-dimethyl-1H-pyrazole (254 mg, 18% yield) as a brown yellow solid. LC-MS (m/z): 284.9 [M+H]⁺.

### Step 4: 4-(2,6-difluorobenzyl)-2-(4-((1,3-dimethyl-1H-pyrazol-5-yl)oxy)-3-fluorophenyl)-2,4-dihydro-3H-1,2,4-triazol-3-one

To a stirred solution of 5-(4-bromo-2-fluorophenoxy)-1,3-dimethylpyrazole, 4-[(2,6-difluorophenyl)methyl]-2H-1,2,4-triazol-3-one (506.3 mg, 0.77 mmol) and 4-[(2,6-difluorophenyl)methyl]-2H-1,2,4-triazol-3-one (250 mg, 1.18 mmol) in Dioxane (20 mL) were added Copper(I) iodide (60.3 mg, 0.59 mmol), Potassium carbonate (262.9 mg, 3.55 mmol) and (1R,2R)—N1,N2-dimethylcyclohexane-1,2-diamine (50.2 mg, 0.59 mmol) at rt. The resulted mixture was stirred for additional 4 h at 100° C. The resulted mixture was diluted with water (40 mL). The resulted mixture was extracted with EtOAc (3×20 mL). The combined organic layers were washed with brine (40 mL), dried over anhydrous Na₂SO₄, and after filtration, the filtrate was concentrated under reduced pressure. The residue was purified by silica gel column chromatography, eluted with (PE:EtOAc=1-20%) to afford 4-(2,6-difluorobenzyl)-2-(4-((1,3-dimethyl-1H-pyrazol-5-yl)oxy)-3-fluorophenyl)-2,4-dihydro-3H-1,2,4-triazol-3-one (80 mg, 21.9% yield) as a white solid. LC-MS (m/z): 416.1 [M+H]⁺. ¹H NMR (400 MHz, CDCl₃) δ 7.92 (dd, J=12.0, 2.5 Hz, 1H), 7.79-7.74 (m, 1H), 7.59 (s, 1H), 7.37 (m, J=8.4 Hz, 1H), 7.19 (t, J=8.8 Hz, 1H), 6.98 (dd, J=8.3, 7.5 Hz, 2H), 5.32 (s, 1H), 4.97 (s, 2H), 3.75 (s, 3H), 2.19 (s, 3H).

| Compound No. | | | Appearance | | MS(m/z) |
|---|---|---|---|---|---|
| Method | Activity | Structure and Name | and Yield | ¹H NMR Data | [M + H]⁺ |
| Example 172 Method 45 | ++++ | 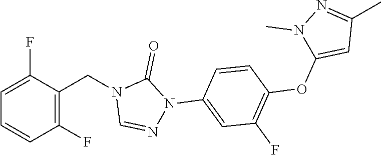 [SMILES: Cc1cc(Oc2ccc(-n3ncn(Cc4c(F)cccc4F)c3=O)cc2F)n(C)n1] 4-(2,6-difluorobenzyl)-2-(4-((1, 3-dimethyl-1H-pyrazol-5-yl)oxy)- 3-fluorophenyl)-2,4-dihydro- 3H-1,2,4-triazol-3-one | white solid, yield: 21.9% | ¹H NMR (400 MHz, CDCl₃) δ 7.92 (dd, J = 12.0, 2.5 Hz, 1H), 7.79-7.74 (m, 1H), 7.59 (s, 1H), 7.37 (m, J = 8.4 Hz, 1H), 7.19 (t, J = 8.8 Hz, 1H), 6.98 (dd, J = 8.3, 7.5 Hz, 2 H), 5.32 (s, 1H), 4.97 (s, 2H), 3.75 (s, 3H), 2.19 (s, 3H). | 416.1 |

### Example 173: Method 46

### methyl 2-(4-(4-(2,6-difluorobenzyl)-5-oxo-4,5-dihydro-1H-1,2,4-triazol-1-yl)-2-fluorophenoxy)-1-methyl-1H-imidazole-4-carboxylate

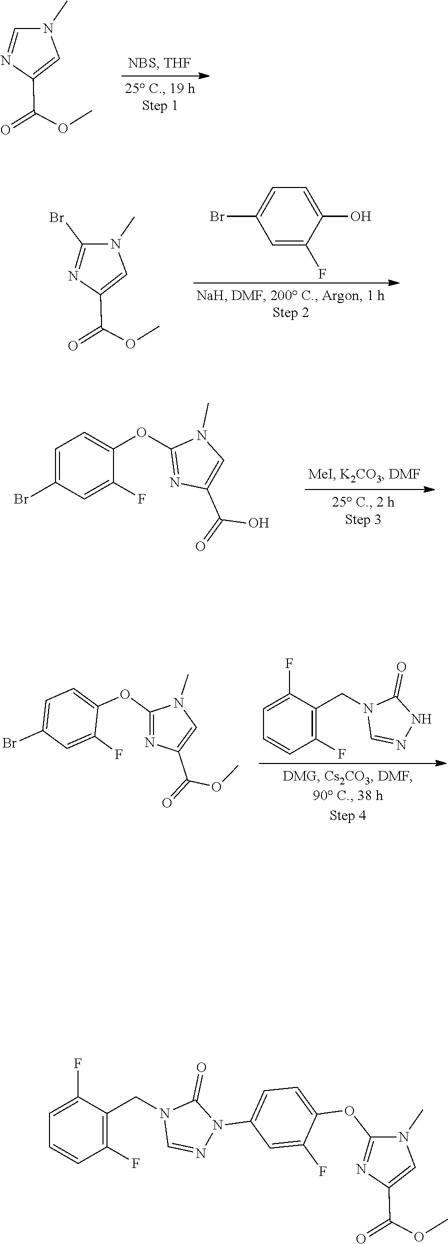

### Step 1: methyl 2-bromo-1-methyl-1H-imidazole-4-carboxylate

To a stirred solution of methyl 1-methyl-1H-imidazole-4-carboxylate (1.4 g, 10 mmol) in THF (20 mL) were added NBS (2.67 g, 15 mmol) at rt. The resulted mixture was stirred for additional 19 h at 25° C. The mixture was concentrated under reduced pressure. The resulted mixture was diluted with water (30 mL). The resulted mixture was extracted with EtOAc (3×15 mL). The combined organic layers were washed with brine (20 mL), dried over anhydrous Na₂SO₄, and after filtration, the filtrate was concentrated under reduced pressure. The residue was purified by silica gel column chromatography, eluted with (PE: EtOAc=1:1) to afford methyl 2-bromo-1-methyl-1H-imidazole-4-carboxylate (1.4 g, 64.0% yield) as a yellow solid. MS (m/z): 218.9, 220.9 [M+H]⁺.

### Step 2: 2-(4-bromo-2-fluorophenoxy)-1-methyl-1H-imidazole-4-carboxylic acid

To a solution of methyl 2-bromo-1-methyl-1H-imidazole-4-carboxylate (600 mg, 2.74 mmol), 4-bromo-2-fluorophenol (523 mg, 2.74 mmol) in DMF (10 mL) was added NaH (131 mg, 3.29 mmol) at r.t. The resulted mixture was stirred for 1 h at 200° C. under microwave conditions. The mixture was allowed to cool down to room temperature. The resulted mixture was diluted with water (100 mL). The aqueous layer was acidified to pH 3-4 with 1N HCl. The resulted mixture was extracted with EtOAc (3×45 mL). The combined organic layers were washed with brine (80 mL), dried over anhydrous Na₂SO₄, and after filtration, the filtrate was concentrated under reduced pressure to afford crude 2-(4-bromo-2-fluorophenoxy)-1-methyl-1H-imidazole-4-carboxylic acid (5 g, 97.0% yield) as a brown oil. MS (m/z): 314.9, 317.0 [M+H]⁺. It was used to the next step reaction.

### Step 3: methyl 2-(4-bromo-2-fluorophenoxy)-1-methyl-1H-imidazole-4-carboxylate

To a stirred solution of 2-(4-bromo-2-fluorophenoxy)-1-methyl-1H-imidazole-4-carboxylic acid (1.9 g, 0.006 mol) in DMF (20 mL) were added K₂CO₃ (2.49 g, 0.018 mol) and MeI (1.02 g, 0.007 mol) at rt. The resulted mixture was diluted with water (100 mL). The resulted mixture was extracted with EtOAc (3×50 mL). The combined organic layers were washed with brine (3×80 mL), dried over anhydrous Na₂SO₄, and after filtration, the filtrate was concentrated under reduced pressure. The residue was purified by silica gel column chromatography, eluted with (PE:EtOAc=1:1) to afford methyl 2-(4-bromo-2-fluorophenoxy)-1-methyl-1H-imidazole-4-carboxylate (0.4 g, 20.0% yield) as a yellow solid. MS (m/z): 329.0, 330.9 [M+H]⁺.

### Step 4: methyl 2-(4-(4-(2,6-difluorobenzyl)-5-oxo-4,5-dihydro-1H-1,2,4-triazol-1-yl)-2-fluorophenoxy)-1-methyl-1H-imidazole-4-carboxylate

To a stirred solution of methyl 2-(4-bromo-2-fluorophenoxy)-1-methyl-1H-imidazole-4-carboxylate (370 mg, 1.12 mmol), 4-(2,6-difluorobenzyl)-2,4-dihydro-3H-1,2,4-triazol-3-one (475 mg 2.25 mmol), N,N-Dimethylglycine (116 mg, 1.12 mmol) and CuI (214 mg 1.12 mmol) in DMF (10 mL) was added Cs₂CO₃ (1099 mg, 3.37 mmol) at rt. The resulted mixture was stirred for additional 38 h at 90° C. under Ar. The resulted mixture was diluted with water (80 mL). The resulted mixture was extracted with EtOAc (3×40 mL). The combined organic layers were washed with brine (60 mL), dried over anhydrous Na₂SO₄, and after filtration, the filtrate was concentrated under reduced pressure. The residue was purified by silica gel column chromatography, eluted with (PE:EtOAc=1:1) to afford the titled compound as a white solid (150 mg, yield: 29.0%). LC-MS (m/z): 460.0 [M+H]⁺. ¹H NMR (400 MHz, DMSO) δ 8.37 (s, 1H), 7.89 (dd, J=12.3, 2.4 Hz, 1H), 7.78-7.72 (m, 2H), 7.58 (t, J=8.9 Hz, 1H), 7.53-7.44 (m, 1H), 7.16 (t, J=8.1 Hz, 2H), 4.99 (s, 2H), 3.67 (s, 3H), 3.62 (s, 3H).

| Com- | | | Appear- | | |
|---|---|---|---|---|---|
| pound No. | | | ance | | MS(m/z) |
| Method | Activity | Structure and Name | and Yield | ¹H NMR Data | [M + H]⁺ |
| Example 173 Method 46 | +++ | 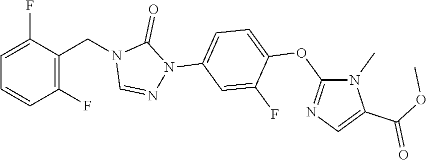 [SMILES: COC(=O)c1cnc(Oc2ccc(-n3ncn(Cc4c(F)cccc4F)c3=O)cc2F)n1C] methyl 2-(4-(4-(2,6-difluorobenzyl)-5- oxo-4,5-dihydro-1H-1,2,4-triazol- 1-yl)-2-fluorophenoxy)-1-methyl- 1H-imidazole-5-carboxylate | white solid, yield: 25.5% | ¹H NMR (400 MHz, CDCl₃) δ 7.98 (dd, J = 12.1, 2.5 Hz, 1H), 7.87 (dd, J = 9.0, 1.6 Hz, 1H), 7.57 (s, 1H), 7.44 (m, 2H), 7.36 (m, 1H), 6.98 (t, J = 7.9 Hz, 2H), 4.96 (s, 2H), 3.87 (s, 3H), 3.84 (s, 3H). | 460.1 |

### Example 174: Method 47

### 2-(4-((2-acetyl-1,4-dimethyl-1H-imidazol-5-yl)oxy)-3-fluorophenyl)-4-(2,6-difluorobenzyl)-2,4-dihydro-3H-1,2,4-triazol-3-one

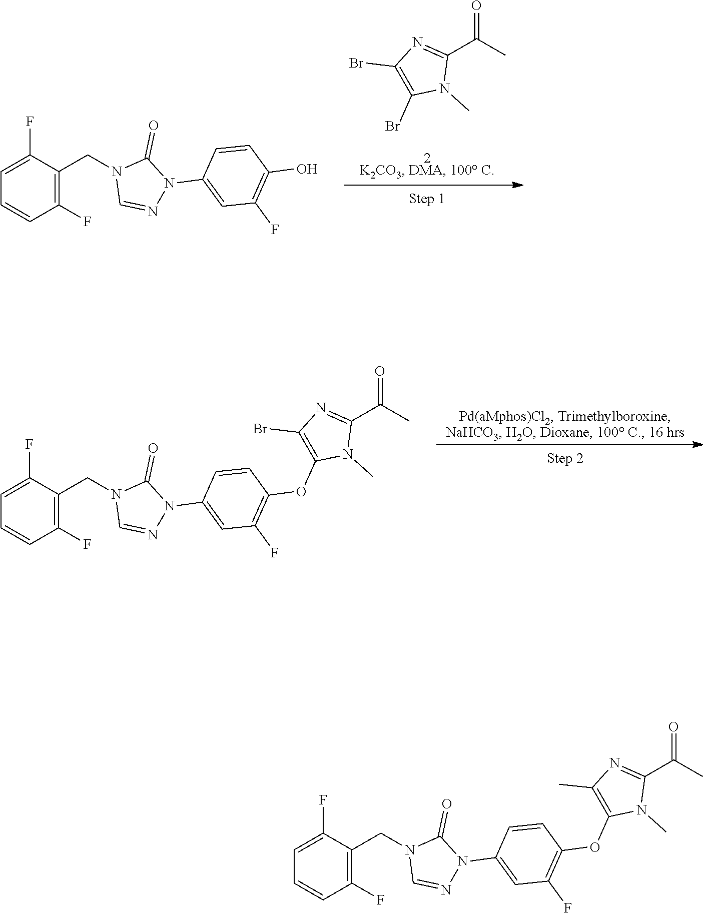

### Step 1: 2-(4-((2-acetyl-4-bromo-1-methyl-1H-imidazol-5-yl)oxy)-3-fluorophenyl)-4-(2,6-difluorobenzyl)-2,4-dihydro-3H-1,2,4-triazol-3-one

To a stirred solution of 4-(2,6-difluorobenzyl)-2-(3-fluoro-4-hydroxyphenyl)-2,4-dihydro-3H-1,2,4-triazol-3-one (1 g, 3.1 mmol), 1-(4,5-dibromo-1-methyl-1H-imidazol-2-yl) ethan-1-one (0.874 g, 3.1 mmol) in DMA (50 mL) was added K₂CO₃ (1.28 g, 9.3 mmol). The resulted mixture was stirred for additional 12 h at 100° C. The resulted mixture was diluted with water (50 mL). The resulted mixture was extracted with EtOAc (3×50 mL). The combined organic layers were washed with brine (40 mL), dried over anhydrous Na₂SO₄, and after filtration, the filtrate was concentrated under reduced pressure. The residue was purified by flash chromatography (PE:EtOAc=1:1) to afford 2-(4-((2-acetyl-4-bromo-1-methyl-1H-imidazol-5-yl)oxy)-3-fluorophenyl)-4-(2,6-difluorobenz yl)-2,4-dihydro-3H-1,2,4-triazol-3-one (220 mg, 13.5% yield) as an yellow solid. LC-MS (m/z): 522.01[M+H]⁺.

### Step 2: 2-(4-((2-acetyl-1,4-dimethyl-1H-imidazol-5-yl)oxy)-3-fluorophenyl)-4-(2,6-difluorobenzyl)-2,4-dihydro-3H-1,2,4-triazol-3-one

To a stirred solution of 2-(4-((2-acetyl-4-bromo-1-methyl-1H-imidazol-5-yl)oxy)-3-fluorophenyl)-4-(2,6-difluorobenzyl)-2,4-dihydro-3H-1,2,4-triazol-3-one (20 mg, 0.038 mmol), Trimethylboroxine (7.1 mg, 0.057 mmol) and NaHCO₃ (9.57 mg, 0.11 mmol) in Dioxane (2 mL) was added Pd(aMphos)Cl₂ (2.7 mg, 0.0038 mmol) under a argon atmosphere. The resulted mixture was stirred for additional 16 h at 100° C. The resulted mixture was diluted with water (2 mL). The resulted mixture was extracted with EtOAc (3×5 mL). The combined organic layers were washed with brine (2 mL), dried over anhydrous Na₂SO₄, and after filtration, the filtrate was concentrated under reduced pressure. The residue was purified by flash chromatography with the following conditions: column: Gemini 5u C18 150×21.2 mm; Mobile phase B: ACN; Flow rate: 20 mL/min; Gradient: 40% B-60% B in 20 min; Detector: 214 nm. The desired fractions were concentrated under reduced pressure to afford the titled compound as a white solid(1.2 mg, 6.9% yield). LC-MS (m/z): 458.1[M+H]⁺.

| Com- | | | Appear- | | |
|---|---|---|---|---|---|
| pound No. | | | ance | | MS(m/z) |
| Method | Activity | Structure and Name | and Yield | ¹H NMR Data | [M + H]⁺ |
| Example 174 Method 47 | ++++ | 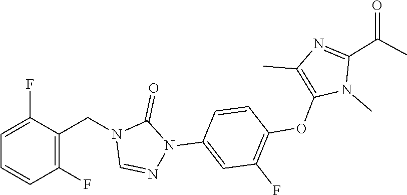 [SMILES: CC(=O)c1nc(C)c(Oc2ccc(-n3ncn(Cc4c(F)cccc4F)c3=O)cc2F)n1C] 2-(4-((2-acetyl-1,4-dimethyl-1H- imidazol-5-yl)oxy)-3-fluorophenyl)- 4-(2,6-difluorobenzyl)-2,4- dihydro-3H-1,2,4-triazol-3-one | white solid, yield: 6.9% | ¹HNMR (400 MHz, CDCl₃) δ 7.93 (dd, J = 12.3, 2.2 Hz, 1H), 7.70 (d, J = 8.6 Hz, 1H), 7.58 (s, 1H), 7.36 (m, 1H), 6.97 (m, 2H), 6.80 (t, J = 8.5 Hz, 1H), 4.96 (s, 2H), 3.80 (s, 3H), 2.63 (s, 3H), 2.05 (s, 3H). | 458.1 |

### Example 175: Method 48

### 2-(4-((1,4-dimethyl-1H-pyrazol-5-yl)oxy)-3-fluorophenyl)-5-phenyl-2,5,6,7-tetrahydro-3H-pyrrolo[2,1-c][1,2,4]triazol-3-one

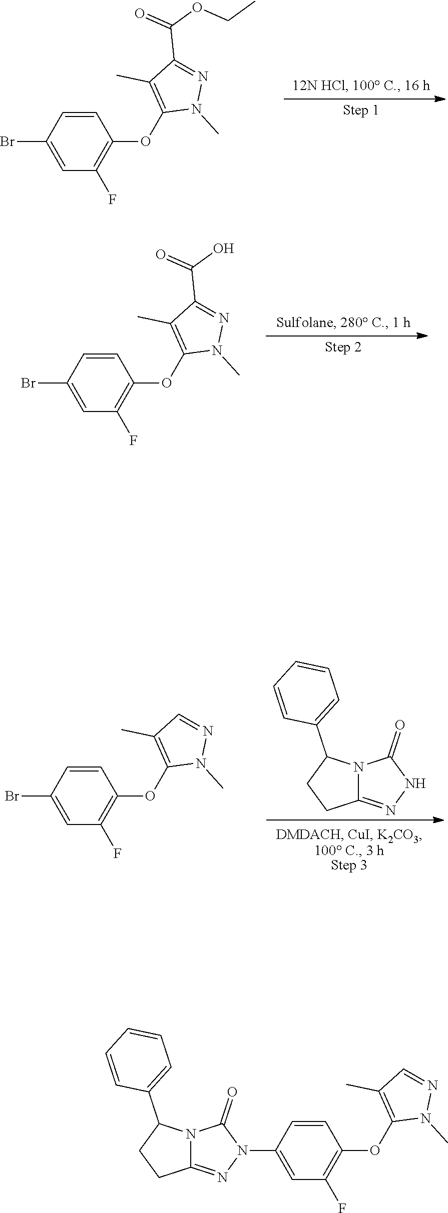

### Step 1: 5-(4-bromo-2-fluorophenoxy)-1,4-dimethyl-1H-pyrazole-3-carboxylic acid

To a stirred solution of ethyl 5-(4-bromo-2-fluorophenoxy)-1,4-dimethylpyrazole-3-Carboxylate (350 mg, 0.98 mmol) in H₂O (1 mL) was added 12N HCl (10 mL). The reaction mixture was stirred at 100° C. for 16 h. The reaction mixture was concentrated under pressure at 50° C. The residue was purified via Flash Chromatography and was eluted with CH₂Cl₂/MeOH (20:1) and give the 5-(4-bromo-2-fluorophenoxy)-1,4-dimethyl-1H-pyrazole-3-carboxylic acid (280 mg, 86.82% yield) as a yellow solid. MS (m/z): 328.9 [M+H]⁺.

### Step 2: 5-(4-bromo-2-fluorophenoxy)-1,4-dimethyl-1H-pyrazole

A solution of 5-(4-bromo-2-fluorophenoxy)-1,4-dimethylpyrazole-3-carboxylic Acid (270 mg, 0.82 mmol) in tetramethylene sulfone (10 mL) was stirred at 280° C. for 1h. The residue was extracted with EA (50 mL). The residue was purified via Flash Chromatography and was eluted with PE/EtOAc (3:1) and give the 5-(4-bromo-2-fluorophenoxy)-1,4-dimethyl-1H-pyrazole (170 mg, 64.13% yield) as a yellow oil. MS (m/z): 284.9 [M+H]⁺.

### Step 3: 2-(4-((1,4-dimethyl-1H-pyrazol-5-yl)oxy)-3-fluorophenyl)-5-phenyl-2,5,6,7-tetrahydro-3H-pyrrolo[2,1-c][1,2,4]triazol-3-one

To a stirred solution of 5-phenyl-2H,5H,6H,7H-pyrrolo[2,1-c][1,2,4]triazol-3-one (90 mg, 0.45 mmol), 5-(4-bromo-2-fluorophenoxy)-1,4-dimethylpyrazole (127 mg, 0.45 mmol), copper(I) iodide (17 mg, 0.09 mmol) and K₂CO₃ (154 mg, 1.12 mmol) in DMF (5 mL) under nitrogen at 25° C. was added a solution of DMDACH (25 mg, 0.18 mmol) in DMF (1 mL) dropwise. The reaction mixture was stirred at 110° C. for 2 h. The reaction mixture was concentrated under pressure at 50° C. The residue was extracted with EA (2×30 mL). The residue was purified via flash chromatography and was eluted with PE/EtOAc (6:1) and give the 2-(4-((1,4-dimethyl-1H-pyrazol-5-yl)oxy)-3-fluorophenyl)-5-phenyl-2,5,6,7-tetrahydro-3H-pyrrolo[2,1-c][1,2,4]triazol-3-one (5.0 mg, 2.75% yield) as a white solid. LC-MS (m/z) 406.0 [M+H]⁺. ¹H NMR (400 MHz, CDCl₃) δ 7.89 (dd, J=12.5, 2.5 Hz, 1H), 7.66 (dd, J=6.5, 4.8 Hz, 1H), 7.43-7.32 (m, 4H), 7.25 (d, J=6.9 Hz, 2H), 6.83 (t, J=8.9 Hz, 1H), 5.30-5.26 (m, 1H), 3.73 (s, 3H), 3.08-2.90 (m, 3H), 2.53 (dd, J=10.0, 5.6 Hz, 1H), 1.79 (s, 3H).

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 175 Method 48 | ++++ | 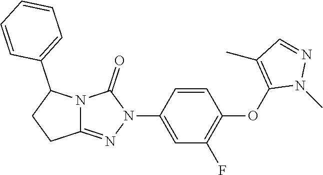 [SMILES: Cc1cnn(C)c1Oc1ccc(-n2nc3n(c2=O)C(c2ccccc2)CC3)cc1F] | white solid, yield: 2.75% | ¹H NMR (400 MHz, CDCl₃) δ 7.89 (dd, J = 12.5, 2.5 Hz, 1H), 7.66 (dd, J = 6.5, 4.8 Hz, 1H), 7.43-7.32 (m, 4H), 7.25 (d, J = 6.9 Hz, 2H), 6.83 (t, J = 8.9 Hz, 1H), 5.30-5.26 (m, 1H), 3.73 (s, 3H), 3.08-2.90 (m, 3H), 2.53 (dd, J = 10.0, 5.6 Hz, 1H), 1.79 (s, 3H). | 406.0 |
| | | 2-(4-((1,4-dimethyl-1H-pyrazol- | | | |
| | | 5-yl)oxy)-3-fluorophenyl)-5- | | | |
| | | phenyl-2,5,6,7-tetrahydro-3H- | | | |
| | | pyrrolo[2,1-c][1,2,4]triazol-3-one | | | |

### Example 176: Method 49

### 4-(2,6-difluorobenzyl)-2-(4-((1,4-dimethyl-1H-pyrazol-5-yl)oxy)phenyl)-2,4-dihydro-3H-1,2,4-triazol-3-one

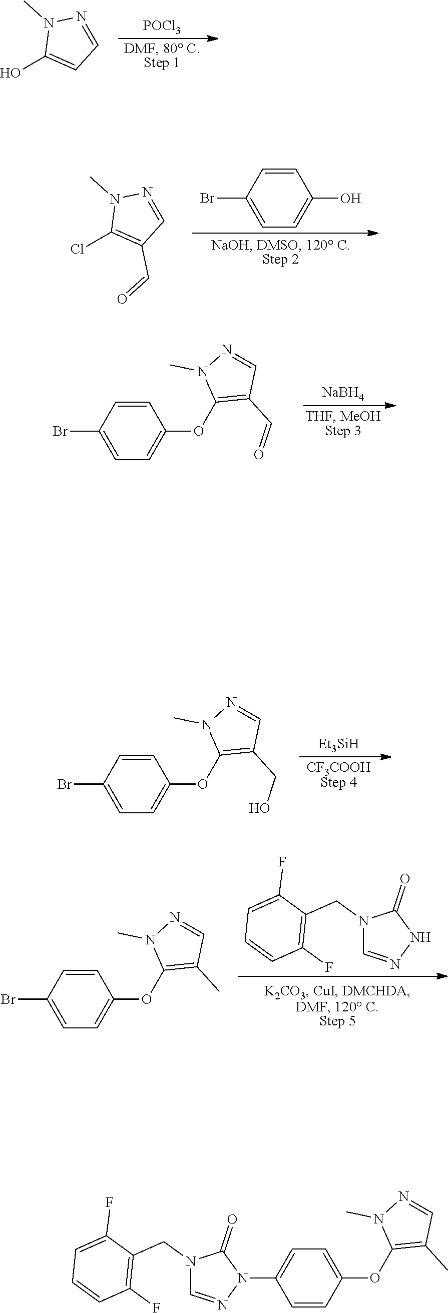

### Step 1: 5-chloro-1-methyl-1H-pyrazole-4-carbaldehyde

DMF (2.98 g, 40.8 mmol) was added to 2-methylpyrazol-3-ol (1 g, 10.2 mmol). Phosphoryl trichloride (12.51 g, 81.6 mmol) was then added dropwise over 10 min. The reaction mixture was stirred at 80° C. for 6 h, then cooled to room temperature, diluted with NaHCO₃ solution, and extracted three times with ethyl acetate (20 mL). The organic layers were combined, the solvent was removed under vacuum and the crude was purified through silica gel chromatography (PE: EA=4:1) to give the desired product as a yellow solid (1.03 g, 63% yield).

### Step 2: 5-(4-bromophenoxy)-1-methyl-1H-pyrazole-4-carbaldehyde

To a stirred solution of 5-chloro-1-methylpyrazole-4-carbaldehyde (1.03 g, 7.1 mmol), 4-bromophenol (1.60 g, 9.23 mmol) in DMSO (20 mL) was added NaOH (0.37 g, 9.23 mmol). The reaction mixture was stirred at 110° C. for 16 h, then cooled to room temperature, diluted with water, and extracted three times with ethyl acetate (50 mL). The organic layers were combined, the solvent was removed under vacuum and the crude was purified through silica gel chromatography (PE: EA=5:1) to give the desired product as a yellow solid (1.39 g, 66% yield). LCMS (m/z): 280.1, 282.0 [M+H]⁺, RT: 1.288 min.

### Step 3: (5-(4-bromophenoxy)-1-methyl-1H-pyrazol-4-yl)methanol

To a solution of 5-(4-bromophenoxy)-1-methylpyrazole-4-carbaldehyde (1.39 g, 4.9 mmol) in THF (10 mL) and MeOH (10 mL) was added NaBH₄(0.2 g, 5.3 mmol). The reaction mixture was stirred at room temperature for 16 h. The reaction was quenched with H₂O (30 mL) and extracted with EA (3×30 mL), the organic layers were combined, the solvent was removed under vacuum and the crude was purified through silica gel chromatography (PE: EA=2:1) to give the desired product as a yellow oil (1.16 g, yield: 80%). LCMS (m/z): 283.0, 285.0 [M+H]⁺,

### Step 4: 5-(4-bromophenoxy)-1,4-dimethyl-1H-pyrazole

To a solution of (5-(4-bromophenoxy)-1-methyl-1H-pyrazol-4-yl)methanol (1.16 g, 4.1 mmol) in CF₃COOH (20 mL) was added Et₃SiH(0.95 g, 8.2 mmol). The reaction mixture was stirred at room temperature for 16 h. Then the reaction was diluted with NaHCO₃ solution, and extracted three times with ethyl acetate (30 mL). The organic layers were combined, the solvent was removed under vacuum and the crude was purified through silica gel chromatography (PE: EA=15:1) to give the desired product as a yellow oil (0.93 g, 80% yield). LCMS (m/z): 267.0, 269.0 [M+H]⁺.

### Step 5: 4-(2,6-difluorobenzyl)-2-(4-((1,4-dimethyl-1H-pyrazol-5-yl)oxy)phenyl)-2,4-dihydro-3H-1,2,4-triazol-3-one

To a stirred solution of 5-(4-bromophenoxy)-1,4-dimethyl-1H-pyrazole (50 mg, 0.19 mmol), 4-(2,6-difluorobenzyl)-2,4-dihydro-3H-1,2,4-triazol-3-one (39.53 mg, 0.19 mmol), (1R,2R)—N, N/′-Dimethyl-1,2-cyclohexanediamine (5.33 mg, 0.037 mmol), Copper(I) iodide (3.57 mg, 0.019 mmol) in DMF(10 mL) was added K₂CO₃ (52 mg, 0.37 mmol). The reaction mixture was stirred at 120° C. for 16 h under N₂ atmosphere. After cooling to rt, 30 mL of water was added to the reaction mixture. The mixture was extracted with EtOAc (3×30 mL). The combined organic layers were dried over Na₂SO₄, filtered and concentrated. The crude product was purified by pre-TLC (PE: EA=2:1) to afford the desired product as a yellow solid (12.2 mg, 16% yield). LCMS (m/z): 398.1 [M+H]⁺. ¹H NMR (400 MHz, MeOH-d₄) δ 7.99 (s, 1H), 7.85 (d, J=9.2 Hz, 2H), 7.41-7.49 (m, 1H), 7.33 (s, 1H), 7.06 (t, 2H), 6.99 (d, J=9.1 Hz, 2H), 5.03 (s, 2H), 3.62 (s, 3H), 1.80 (s, 3H).

| | | | | | |
|---|---|---|---|---|---|
| Example 176 Method 49 | ++++ | 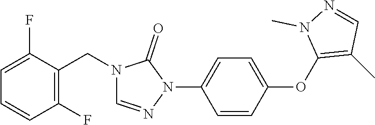 [SMILES: Cc1cnn(C)c1Oc1ccc(-n2ncn(Cc3c(F)cccc3F)c2=O)cc1] | white solid, yield: 16% | ¹H NMR (400 MHz, MeOH-d₄) δ 7.99 (s, 1H), 7.85 (d, J = 9.2 Hz, 2H), 7.41-7.49 (m, 1H), 7.33 (s, 1H), 7.06 (t, 2H), 6.99 (d, J = 9.1 Hz, 2H), 5.03 (s, 2H), 3.62 (s, 3H), 1.80 (s, 3H). | 398.1 |
| | | 5-(2-fluoro-4-(3-oxo-5-phenyl- | | | |
| | | 6,7-dihydro-3H-pyrrolo[2,1-c] | | | |
| | | [1,2,4]triazol-2(5H)-yl)phenoxy)- | | | |
| | | 1-methyl-1H-pyrazole-3- | | | |
| | | carboxamide | | | |

### Example 177: Method 50

### 4-(2,6-difluorobenzyl)-2-(4-((3,5-dimethyl-1-(oxetan-3-yl)-1H-pyrazol-4-yl)oxy)-3-fluorophenyl)-2,4-dihydro-3H-1,2,4-triazol-3-one

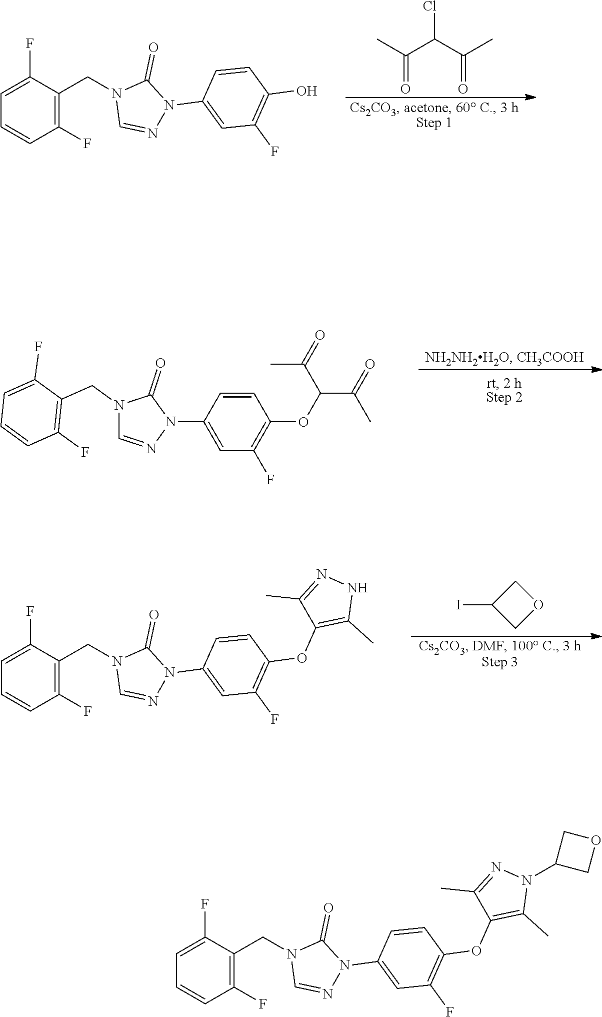

### Step 1: 3-(4-(4-(2,6-difluorobenzyl)-5-oxo-4,5-dihydro-1H-1,2,4-triazol-1-yl)-2-fluorophenoxy)pentane-2,4-dione

To a stirred solution of 4-(2,6-difluorobenzyl)-2-(3-fluoro-4-hydroxyphenyl)-2,4-dihydro-3H-1,2,4-triazol-3-one (2 g, 6.22 mmol) and 3-chloropentane-2,4-dione (1.1 g, 2.37 mmol) in Aceton (50 mL) was added Cs₂CO₃ (3042 mg, 9.33 mmol) at rt. The resulted mixture was stirred for additional 5 h at 60° C. The mixture was allowed to cool down to rt. The resulted mixture was diluted with water (50 mL). The resulted mixture was extracted with EtOAc (3×30 mL). The combined organic layers were washed with brine (20 mL), dried over anhydrous Na₂SO₄, and after filtration, the filtrate was concentrated under reduced pressure. The residue was purified by silica gel column chromatography, eluted with (PE:EtOAc) (1:1) to afford 4-(2,6-difluorobenzyl)-2-(4-nitrophenyl)-2,4-dihydro-3H-1,2,4-triazol-3-one (1300 mg, 49.8% yield) as a brown yellow oil. LCMS (m/z): 420.1 [M+H]⁺.

### Step 2: 4-(2,6-difluorobenzyl)-2-(4-((3,5-dimethyl-1H-pyrazol-4-yl)oxy)-3-fluorophenyl)-2,4-dihydro-3H-1,2,4-triazol-3-one

NH₂NH₂·H₂O (235 mg, 4.65 mmol) was added to a solution of 3-(4-{4-[(2,6-difluorophenyl)methyl]-5-oxo-1,2,4-triazol-1-yl}-2-fluorophenoxy)pentane-2,4-dione(1300 mg, 3.1 mmol) in CH₃COOH (30 mL), and the mixture was stirred at rt for 2 h. The solvent was then removed under vacuum. The residue was purified by silica gel column chromatography, eluted with (PE:EtOAc) (1:1) to afford 4-(2,6-difluorobenzyl)-2-(4-((3,5-dimethyl-1H-pyrazol-4-yl)oxy)-3-fluorophenyl)-2,4-dihydro-3H-1,2,4-triazol-3-one (1000 mg, 77.7%) as a white solid. LCMS (m/z): 416.1 [M+H]⁺. ¹H NMR (400 MHz, CDCl₃) δ 7.83 (m, 1H), 7.62-7.56 (m, 2H), 7.36 (m, 1H), 6.98 (m, 2H), 6.75 (m, 1H), 4.97 (s, 2H), 2.14 (s, 6H).

### Step 3: 4-(2,6-difluorobenzyl)-2-(4-((3,5-dimethyl-1-(oxetan-3-yl)-1H-pyrazol-4-yl)oxy)-3-fluorophenyl)-2,4-dihydro-3H-1,2,4-triazol-3-one

To a stirred solution of 4-[(2,6-difluorophenyl)methyl]-2-{4-[(3,5-dimethyl-1H-pyrazol-4-yl)oxy]-3-fluorophenyl}-1,2,4-triazol-3-one (100 mg, 0.24 mmol) and 3-iodooxetane (133 mg, 0.72 mmol) in DMF (5 mL) was added Cs₂CO₃ (314 mg, 0.96 mmol) at rt. The resulted mixture was stirred for additional 16 h at 100° C. The mixture was allowed to cool down to rt, diluted with water (30 mL), and extracted three times with ethyl acetate (20 mL). The organic layers were combined, the solvent was removed under vacuum and the crude was purified by Prep-HPLC (column-Gemini —C₁₈ 150×21.2 mm, Sum; Mobile phase: ACN-H₂O (0.1% FA), 5%-20% to afford the desired product as a white solid (10.8 mg, 9.5%). LCMS (m/z): 472.1 [M+H]⁺. ¹H NMR (400 MHz, CDCl₃) δ 7.83 (m, 1H), 7.62-7.58 (m, 1H), 7.56 (s, 1H), 7.41-7.32 (m, 1H), 6.99 (m, 2H), 6.72 (m, 1H), 5.36 (d, J=6.8 Hz, 1H), 5.21 (m, 2H), 5.00-4.96 (m, 4H), 2.12 (d, J=9.2 Hz, 6H).

The synthesis procedure of Examples (Compounds) 178-179 were similar to the procedure of Example (Compound) 177.

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 177 Method 50 | ++++ | 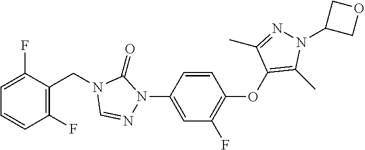 [SMILES: Cc1nn(C2COC2)c(C)c1Oc1ccc(-n2ncn(Cc3c(F)cccc3F)c2=O)cc1F] | white solid, yield: 9.5% | ¹H NMR (400 MHz, CDCl₃) δ 7.83 (m, 1H), 7.62-7.58 (m, 1H), 7.56 (s, 1H), 7.41-7.32 (m, 1H), 6.99 (m, 2H), 6.72 (m, 1H), 5.36 (d, J = 6.8 Hz, 1H), 5.21 (m, 2H), 5.00-4.96 (m, 4H), 2.12 (d, J = 9.2 Hz, 6H). | 472.1 |
| | | 4-(2,6-difluorobenzyl)-2-(4-((3,5- | | | |
| | | dimethyl-1-(oxetan-3-yl)-1H- | | | |
| | | pyrazol-4-yl)oxy)-3-fluorophenyl)- | | | |
| | | 2,4-dihydro-3H-1,2,4-triazol- | | | |
| | | 3-one | | | |
| Example 178 Method 50 | ++++ | 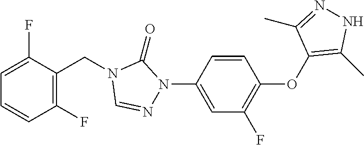 [SMILES: Cc1n[nH]c(C)c1Oc1ccc(-n2ncn(Cc3c(F)cccc3F)c2=O)cc1F] | white solid, yield: 77.7% | ¹H NMR (400 MHz, CDCl₃) δ 7.83 (m, 1H), 7.62-7.56 (m, 2H), 7.36 (m, 1H), 6.98 (m, 2H), 6.75 (m, 1H), 4.97 (s, 2H), 2.14 (s, 6H). | 416.1 |
| | | 4-(2,6-difluorobenzyl)-2-(4-((3,5- | | | |
| | | dimethyl-1H-pyrazol-4-yl)oxy)- | | | |
| | | 3-fluorophenyl)-2,4-dihydro-3H- | | | |
| | | 1,2,4-triazol-3-one | | | |
| Example 179 Method 50 | ++++ | 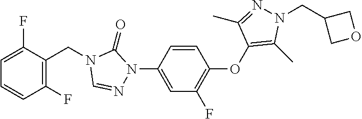 [SMILES: Cc1nn(CC2COC2)c(C)c1Oc1ccc(-n2ncn(Cc3c(F)cccc3F)c2=O)cc1F] | white solid, yield: 26.1% | ¹H NMR (400 MHz, CDCl3) δ 7.82 (m, 1H), 7.61-7.55 (m, 2H), 7.39-7.33 (m, 1H), 6.98 (m, 2H), 6.70 (m, 1H), 4.96 (s, 2H), 4.83 (m, 2H), 4.52 (m, 2H), 4.26 (d, J = 7.5 Hz, 2H), 3.56-3.49 (m, 1H), 2.13 (s, | 486.1 |
| | | 4-(2,6-difluorobenzyl)-2-(4-((3,5- | | 3H), 2.04 (s, 3H). | |
| | | dimethyl-1-(oxetan-3-ylmethyl)- | | | |
| | | 1H-pyrazol-4-yl)oxy)-3-fluoro- | | | |
| | | phenyl)-2,4-dihydro-3H-1,2,4- | | | |
| | | triazol-3-one | | | |

### Example 180: Method 51

### 4-(2,6-difluorobenzyl)-2-(3-fluoro-4-((1-(2-hydroxyethyl)-3,5-dimethyl-1H-pyrazol-4-yl)ox y)phenyl)-2,4-dihydro-3H-1,2,4-triazol-3-one

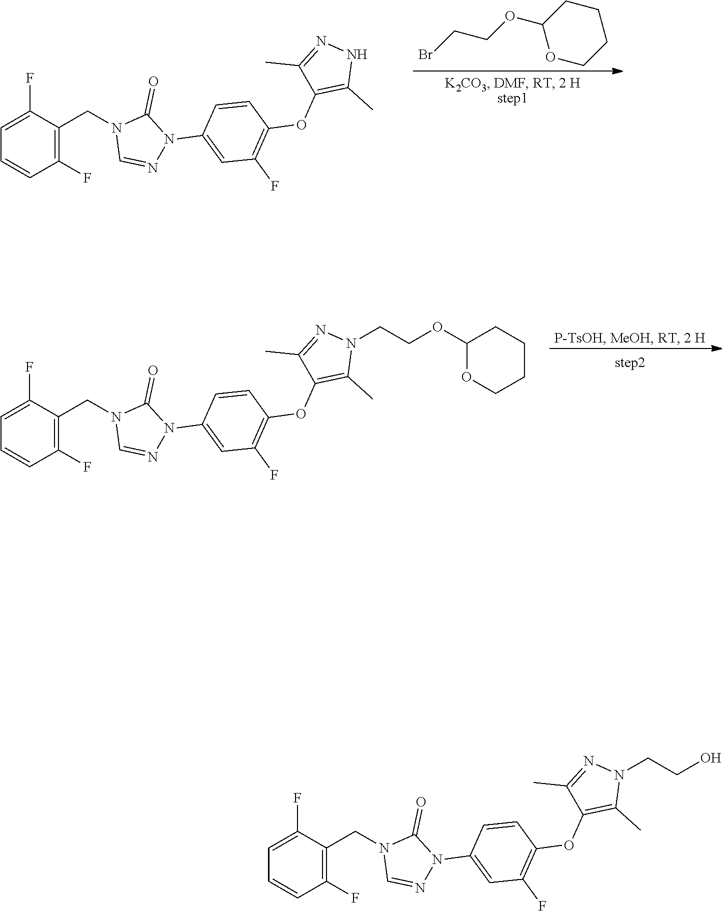

### Step 1: 4-(2,6-difluorobenzyl)-2-(4-((3,5-dimethyl-1-(2-((tetrahydro-2H-pyran-2-yl)oxy)ethyl)-1H-pyrazol-4-yl)oxy)-3-fluorophenyl)-2,4-dihydro-3H-1,2,4-triazol-3-one

To a stirred solution of 4-(2,6-difluorobenzyl)-2-(4-((3,5-dimethyl-1H-pyrazol-4-yl)oxy)-3-fluorophenyl)-2,4-dihydro-3H-1,2,4-triazol-3-one (0.1 g, 0.24 mmol) and 2-(2-bromoethoxy)tetrahydro-2H-pyran (55 mg, 0.26 mmol) in DMF (10 mL) was added K₂CO₃ (72 mg, 0.52 mmol) at rt. The resulted mixture was stirred for additional 2 h at 25° C. The resulted mixture was diluted with water (50 mL). The resulted mixture was extracted with EtOAc (3×30 mL). The combined organic layers were washed with brine (20 mL), dried over anhydrous Na₂SO₄, and after filtration, the filtrate was concentrated under reduced pressure. The residue was purified by silica gel column chromatography, eluted with (PE:EtOAc) (1:1) to afford 4-(2,6-difluorobenzyl)-2-(4-((3,5-dimethyl-1-(2-((tetrahydro-2H-pyran-2-yl)oxy)ethyl)-1H-pyrazol-4-yl)oxy)-3-fluorophenyl)-2,4-dihydro-3H-1,2,4-triazol-3-one (120 mg, 91.7% yield) as a white solid. LCMS (m/z): 544.2 [M+H]⁺

### Step 2: 4-(2,6-difluorobenzyl)-2-(3-fluoro-4-((1-(2-hydroxyethyl)-3,5-dimethyl-1H-pyrazol-4-yl)oxy)phenyl)-2,4-dihydro-3H-1,2,4-triazol-3-one

p-Toluenesulfonic acid(57 mg, 0.33 mmol) was added to a solution of 4-[(2,6-difluorophenyl)methyl]-2-[4-({3,5-dimethyl-1-[2-(oxan-2-yloxy)ethyl]pyrazol-4-yl}ox y)-3-fluorophenyl]-1,2,4-triazol-3-one(120 mg, 0.22 mmol) in MeOH(10 mL). The mixture was stirred at rt for 2h., then diluted with water (30 mL), and extracted three times with ethyl acetate (20 mL). The organic layers were combined, the solvent was removed under vacuum and the crude was purified by Prep-HPLC (column-Gemini —C18 150×21.2 mm, Sum; Mobile phase: ACN-H₂O (0.1% FA), 5%-20%) to afford the desired product as a white solid (28.4 mg, 27.99% yield). LCMS (m/z): 460.1 [M+H]⁺. ¹H NMR (400 MHz, CDCl₃) δ 7.86 (m, 1H), 7.64 (m, 1H), 7.56 (s, 1H), 7.40-7.33 (m, 1H), 6.98 (m, 2H), 6.78 (m, 1H), 4.97 (s, 2H), 4.27 (s, 2H), 4.08-4.03 (m, 2H), 2.18 (d, J=6.3 Hz, 6H).

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 180 Method 51 | ++++ | 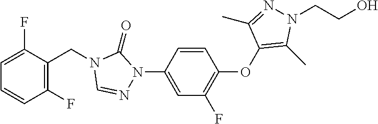 [SMILES: Cc1nn(CCO)c(C)c1Oc1ccc(-n2ncn(Cc3c(F)cccc3F)c2=O)cc1F] | white solid, yield: 28% | ¹H NMR (400 MHz, CDCl₃) δ 7.86 (m, 1H), 7.64 (m, 1H), 7.56 (s, 1H), 7.40-7.33 (m, 1H), 6.98 (m, 2H), 6.78 (m, 1H), 4.97 (s, 2H), 4.27 (s, 2H), 4.08-4.03 (m, 2H), 2.18 (d, J = 6.3 Hz, 6H). | 460.1 |
| | | 4-(2,6-difluorobenzyl)-2-(3-fluoro- | | | |
| | | 4-((1-(2-hydroxyethyl)-3,5-di- | | | |
| | | methyl-1H-pyrazol-4-yl)oxy) | | | |
| | | phenyl)-2,4-dihydro-3H-1,2,4- | | | |
| | | triazol-3-one | | | |

### Example 181: Method 52

### Ethyl-2-(4-(4-(2,6-difluorobenzyl)-5-oxo-4,5-dihydro-1H-1,2,4-triazol-1-yl)-2-fluoropheno xy)-1,5-dimethyl-1H-imidazole-4-carboxylate

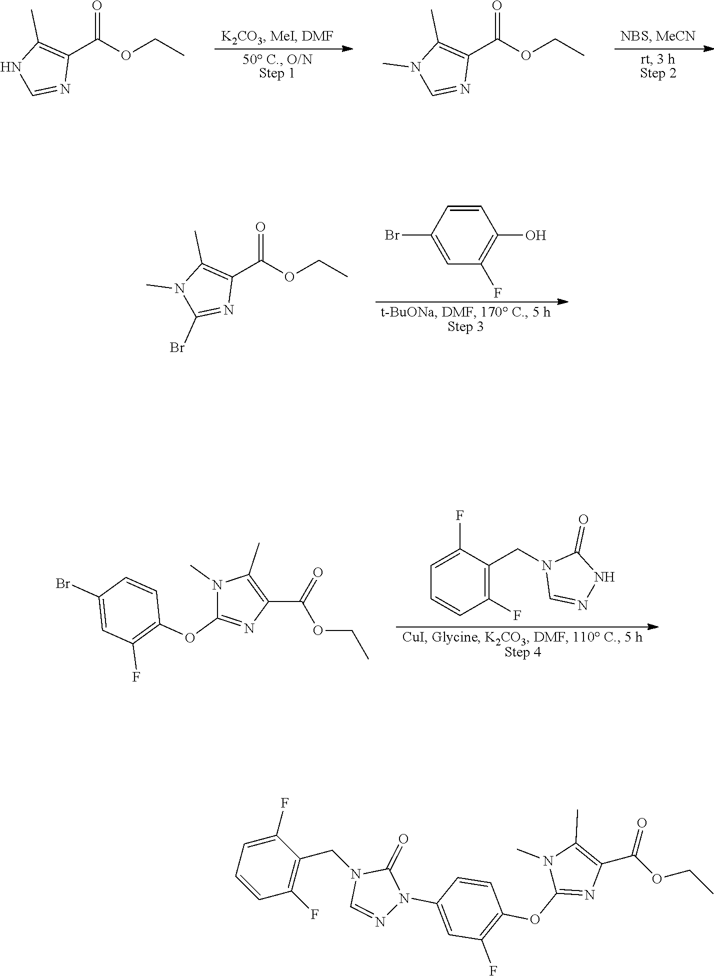

### Step 1: Ethyl-1,5-dimethyl-1H-imidazole-4-carboxylate

A solution of ethyl-5-methyl-1H-imidazole-4-carboxylate (5 g, 32.4 mmol), CH₃I (5.52 g, 38.9 mmol) and K₂CO₃ (5.37 g, 38.9 mmol) in DMF (60 mL) under N₂ atmosphere was stirred at 50° C. for O/N. The mixture was allowed to cool down to rt and was diluted with EtOAc (200 mL). The resulted solution was washed with water (3*50 mL) and brine (50 mL), dried over anhydrous Na₂SO₄, and after filtration, the filtrate was concentrated under reduced pressure. The residue was purified by silica gel column chromatography, eluted with (PE/EtOAc) (3:1) to afford ethyl-1,5-dimethyl-1H-imidazole-4-carboxylate (2 g, 36.7% yield) as a white solid. MS (m/z): 169.2[M+H]⁺.

### Step 2: Ethyl-2-bromo-1,5-dimethyl-1H-imidazole-4-carboxylate

To a stirred solution of ethyl-1,5-dimethyl-1H-imidazole-4-carboxylate (2 g, 11.9 mmol) in MeCN (20 mL), under N₂ atmosphere, was added NBS (2.12 g, 1.39 mmol) at 0° C. Then the mixture solution was stirred at rt for 3 h. The reaction was quenched with a saturated NaHCO₃ solution and was diluted with EtOAc (50 mL). The resulted solution was washed with water (20 mL) and brine (20 mL), dried over anhydrous Na₂SO₄, and after filtration, the filtrate was concentrated under reduced pressure. The residue was purified by silica gel column chromatography, eluted with (PE/EtOAc) (3:1) to afford ethyl-2-bromo-1,5-dimethyl-1H-imidazole-4-carboxylate (1.8 g, 61.5% yield) as a white solid. MS (m/z): 247.2/249.2[M+H]⁺.

### Step 3: Ethyl-2-(4-bromo-2-fluorophenoxy)-1,5-dimethyl-1H-imidazole-4-carboxylate

To a stirred solution of ethyl-2-bromo-1,5-dimethyl-1H-imidazole-4-carboxylate (1.8 g, 7.32 mmol) and 4-bromo-2-fluorophenol (1.39 g, 7.32 mmol) in DMF (30 mL) was added t-BuONa (1.05 g, 10.9 mmol). Then the mixture solution was stirred at 170° C. for 5 h. The mixture was allowed to cool down to rt and was diluted with EtOAc (60 mL). The resulted solution was washed with water (3*30 mL) and brine (30 mL), dried over anhydrous Na₂SO₄, and after filtration, the filtrate was concentrated under reduced pressure. The residue was purified by silica gel column chromatography, eluted with (PE/EtOAc) (2:1) to afford ethyl-2-(4-bromo-2-fluorophenoxy)-1,5-dimethyl-1H-imidazole-4-carboxylate (900 mg, 34.5% yield) as a yellow solid MS (m/z): 357.2[M+H]⁺.

### Step 4: Ethyl-2-(4-(4-(2,6-difluorobenzyl)-5-oxo-4,5-dihydro-1H-1,2,4-triazol-1-yl)-2-fluorophenoxy)-1,5-dimethyl-1H-imidazole-4-carboxylate

A solution of ethyl 2-(4-bromo-2-fluorophenoxy)-1,5-dimethyl-1H-imidazole-4-carboxylate (800 mg, 2.25 mmol), CuI (86 mg, 0.45 mmol), N,N-Dimethylglycine (46 mg, 0.45 mmol), and K₂CO₃ (466 mg, 3.38 mmol) in DMF (20 mL), under N₂ atmosphere, was stirred at 110° C. for 5 h. The resulted mixture was filtrated by kieselguhr, and after filtration, the filtrate was concentrated under reduced pressure. The residue was purified by silica gel column chromatography, eluted with (DCM/MeOH) (30:1) to afford the titled compound (350 mg, 32% yield) as a white solid. MS (m/z): 488.2 [M+H]⁺. ¹H NMR (400 MHz, CDCl₃) δ 7.92 (dd, J=12.4, 2.4 Hz, 1H), 7.78 (d, J=9.2 Hz, 1H), 7.63-7.55 (m, 1H), 7.43-7.32 (m, 2H), 7.02-6.95 (m, 2H), 4.97 (s, 2H), 4.31 (q, J=7.2 Hz, 2H), 3.52 (s, 3H), 2.53 (s, 3H), 1.34 (t, J=7.2 Hz, 3H).

The synthesis procedure of Examples (Compounds) 182-183 were similar to the procedure of Example 181.

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 181 Method 52 | +++ | 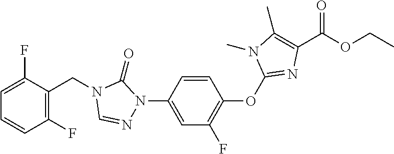 [SMILES: CCOC(=O)c1nc(Oc2ccc(-n3ncn(Cc4c(F)cccc4F)c3=O)cc2F)n(C)c1C] | white solid, yield: 32% | ¹H NMR (400 MHz, CDCl₃) δ 7.92 (dd, J = 12.4, 2.4 Hz, 1H), 7.78 (d, J = 9.2 Hz, 1H), 7.63- 7.55 (m, 1H), 7.43-7.32 (m, 2H), 7.02-6.95 (m, 2H), 4.97 (s, 2H), 4.31 (q, J = 7.2 Hz, 2H), 3.52 (s, 3H), 2.53 (s, 3H), 1.34 (t, J = 7.2 Hz, 3H). | 488.2 |
| | | ethyl | | | |
| | | 2-(4-(4-(2,6-difluorobenzyl)-5- | | | |
| | | oxo-4,5-dihydro-1H-1,2,4-triazol- | | | |
| | | 1-yl)-2-fluorophenoxy)-1,5-di- | | | |
| | | methyl-1H-imidazole-4-carboxylate | | | |
| Example 182 Method 52 | +++ | 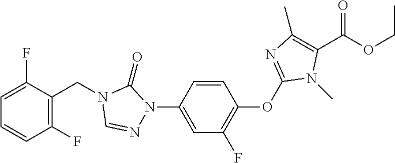 [SMILES: CCOC(=O)c1c(C)nc(Oc2ccc(-n3ncn(Cc4c(F)cccc4F)c3=O)cc2F)n1C] | white solid, yield: 31.3% | ¹H NMR (400 MHz, CDCl₃) δ 7.92 (dd, J = 12.4, 2.4 Hz, 1H), 7.78 (d, J = 9.2 Hz, 1H), 7.63- 7.55 (m, 1H), 7.43-7.32 (m, 2H), 7.02-6.95 (m, 2H), 4.97 (s, 2H), 4.26 (q, J = 7.2 Hz, 2H), 3.61 (s, 3H), 2.56 (s, 3H), 1.31 (t, J = 7.2 Hz, 3H). | 488.2 |
| | | ethyl | | | |
| | | 2-(4-(4-(2,6-difluorobenzyl)-5-oxo- | | | |
| | | 4,5-dihydro-1H-1,2,4-triazol- | | | |
| | | 1-yl)-2-fluorophenoxy)-1,4-di- | | | |
| | | methyl-1H-imidazole-5-carboxylate | | | |
| Example 183 Method 52 | ++++ | 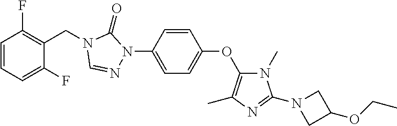 [SMILES: CCOC1CN(c2nc(C)c(Oc3ccc(-n4ncn(Cc5c(F)cccc5F)c4=O)cc3)n2C)C1] | yellow solid, yield: 26% | ¹H NMR (400 MHz, CDCl₃) δ ppm 7.93 (d, J = 8.9 Hz, 2H), 7.57 (s, 1H), 7.36 (s, 1H), 6.96 (m, 4H), 4.97 (s, 2H), 4.56 (t, J = 6.9 Hz, 2H), 4.43 (s, 1H), 4.24 (d, J = 4.9 Hz, 2H), 3.47 | 497.2 |
| | | 4-(2,6-difluorobenzyl)-2-(4-((2- | | (q, J = 7.0 Hz, 2H), 3.22 | |
| | | (3-ethoxyazetidin-1-yl)-1,4-di- | | (s, 3H), 2.07 (s, 3H), | |
| | | methyl-1H-imidazol-5-yl)oxy)phenyl)- | | 1.22 (s, 3H). | |
| | | 2,4-dihydro-3H-1,2,4-triazol- | | | |
| | | 3-one | | | |

### Example 184: Method 53

### 2-(4-((4-bromo-3-methyl-1H-pyrazol-1-yl)methyl)phenyl)-4-(2,6-difluorobenzyl)-2,4-dihydro-3H-1,2,4-triazol-3-one

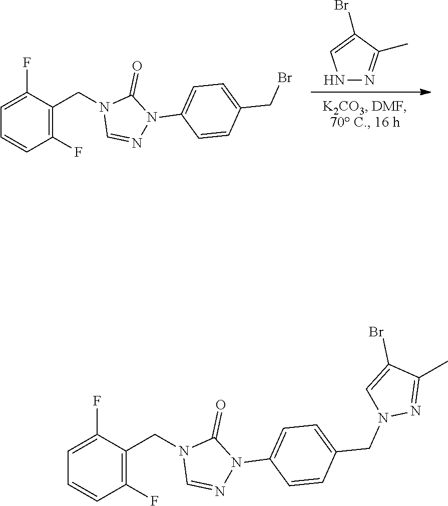

A solution of 2-[4-(bromomethyl)phenyl]-4-[(2,6-difluorophenyl)methyl]-1,2,4-triazol-3-one (50 mg, 0.13 mmol), 4-bromo-3-methyl-1H-pyrazole (25 mg, 0.15 mmol), and K₂CO₃ (36 mg, 0.26 mmol) in DMF (4 mL) was stirred at 70° C. for 16 h. The resulted mixture was diluted with water (30 mL). The resulted mixture was extracted with EtOAc (3×20 mL). The combined organic layers were washed with brine (30 mL), dried over anhydrous Na₂SO₄, and after filtration, the filtrate was concentrated under reduced pressure. The residue was purified by Chiral Prep-HPLC to afford the titled product (8.1 mg, 18.4% yield) as a white solid.MS (m/z): 460.0 [M+H]⁺. ¹H NMR (400 MHz, CDCl₃) δ 7.99-7.93 (m, 2H), 7.58 (s, 1H), 7.35 (d, J=8.4 Hz, 1H), 7.29-7.26 (m, 3H), 7.02-6.94 (m, 2H), 5.19 (s, 2H), 4.97 (s, 2H), 2.24 (s, 3H). The synthesis procedure of Examples (Compounds) 185-211 was similar to the procedure of Example 184.

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 184 Method 53 | ++++ | 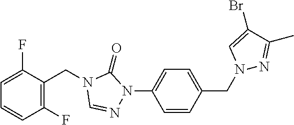 [SMILES: Cc1nn(Cc2ccc(-n3ncn(Cc4c(F)cccc4F)c3=O)cc2)cc1Br] | white solid, yield: 18.4% | ¹H NMR (400 MHz, CDCl₃) δ 7.99-7.93 (m, 2H), 7.58 (s, 1H), 7.35 (d, J = 8.4 Hz, 1H), 7.29-7.26 (m, 3H), 7.02- 6.94 (m, 2H), 5.19 (s, 2H), 4.97 (s, 2H), 2.24 (s, 3H). | 460.0 |
| | | 2-(4-((4-bromo-3-methyl-1H- | | | |
| | | pyrazol-1-yl)methyl)phenyl)-4-(2,6- | | | |
| | | difluorobenzyl)-2,4-dihydro-3H- | | | |
| | | 1,2,4-triazol-3-one | | | |
| Example 185 Method 53 | +++ | 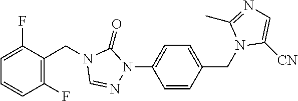 [SMILES: Cc1ncc(C#N)n1Cc1ccc(-n2ncn(Cc3c(F)cccc3F)c2=O)cc1] | White solid, yield: 11.7% | ¹H NMR (400 MHz, CDCl₃) δ 8.27 (s, 1H), 7.86-7.80 (m, 2H), 7.76 (s, 1H), 7.43 (d, J = 8.4 Hz, 1H), 7.22-7.17 (m, 2H), 7.12 (t, J = | 407.1 |
| | | 1-(4-(4-(2,6-difluorobenzyl)-5-oxo- | | 8.1 Hz, 2H), 5.28 (s, 2H), | |
| | | 4,5-dihydro-1H-1,2,4-triazol-1- | | 4.93 (s, 2H), 2.36 (s, 3H). | |
| | | yl)benzyl)-2-methyl-1H-imidazole- | | | |
| | | 5-carbonitrile | | | |
| Example 186 Method 53 | ++++ | 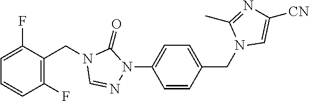 [SMILES: Cc1nc(C#N)cn1Cc1ccc(-n2ncn(Cc3c(F)cccc3F)c2=O)cc1] | white solid, yield: 9.4% | ¹H NMR (400 MHz, CDCl₃) δ 8.27 (s, 1H), 8.11 (s, 1H), 7.86- 7.81 (m, 2H), 7.44 (t, J = 8.4 Hz, 1H), 7.28-7.24 (m, 2H), | 407.1 |
| | | 1-(4-(4-(2,6-difluorobenzyl)-5-oxo- | | 7.12 (t, J = 8.1 Hz, | |
| | | 4,5-dihydro-1H-1,2,4-triazol-1- | | 2H), 5.19 (s, 2H), | |
| | | yl)benzyl)-2-methyl-1H-imidazole- | | 4.93 (s, 2H), 2.23 (s, | |
| | | 4-carbonitrile | | 3H). | |
| Example 187 Method 53 | +++ | 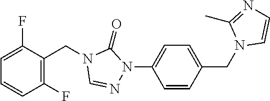 [SMILES: Cc1nccn1Cc1ccc(-n2ncn(Cc3c(F)cccc3F)c2=O)cc1] | white solid, yield: 22.4% | ¹H NMR (400 MHz, CDCl₃) δ 7.97 (d, J = 8.6 Hz, 2H), 7.58 (s, 1H), 7.42-7.32 (m, 1H), 7.12 (d, J = 8.6 Hz, 2H), 7.02-6.94 (m, 3H), 6.85 | 382.1 |
| | | 4-(2,6-difluorobenzyl)-2-(4-((2- | | (s, 1H), 5.06 (s, 2H), | |
| | | methyl-1H-imidazol-1-yl)methyl) | | 4.97 (s, 2H), 2.39 (s, 3H). | |
| | | phenyl)-2,4-dihydro-3H-1,2,4- | | | |
| | | triazol-3-one | | | |
| Example 188 Method 53 | +++ | 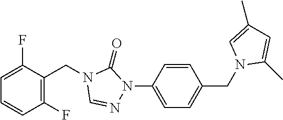 [SMILES: Cc1cc(C)n(Cc2ccc(-n3ncn(Cc4c(F)cccc4F)c3=O)cc2)c1] | off-white solid, yield: 29.6% | ¹H NMR (400 MHz, DMSO-d₆) δ 8.26 (s, 1H), 7.75-7.67 (m, 2H), 7.52- 7.45 (m, 2H), 7.21-7.12 (m, 5H), 4.96 (s, 2H), 3.74 (s, 2H), 2.06 (s, 3H), 1.88 (s, 3H). | 395.1 |
| | | 4-(2,6-difluorobenzyl)-2-(4-((2,4- | | | |
| | | dimethyl-1H-pyrrol-1-yl)methyl) | | | |
| | | phenyl)-2,4-dihydro-3H-1,2,4- | | | |
| | | triazol-3-one | | | |
| Example 189 Method 53 | ++++ | 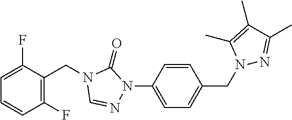 [SMILES: Cc1nn(Cc2ccc(-n3ncn(Cc4c(F)cccc4F)c3=O)cc2)c(C)c1C] | off-white solid, yield: 29.6% | ¹H NMR (400 MHz, DMSO-d₆) δ 8.28 (s, 1H), 7.79 (d, J = 8.6 Hz, 2H), 7.53-7.40 (m, 1H), 7.19- 7.09 (m, 4H), 5.16 (s, 2H), 4.96 (s, 2H), 2.06 (s, 3H), 2.04 (s, 3H), 1.83 (s, 3H). | 410.0 |
| | | 4-(2,6-difluorobenzyl)-2-(4-((3,4,5- | | | |
| | | trimethyl-1H-pyrazol-1-yl)methyl) | | | |
| | | phenyl)-2,4-dihydro-3H-1,2,4- | | | |
| | | triazol-3-one | | | |
| Example 190 Method 53 | ++++ | 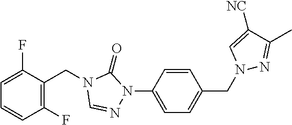 [SMILES: Cc1nn(Cc2ccc(-n3ncn(Cc4c(F)cccc4F)c3=O)cc2)cc1C#N] | white solid, yield: 25.2% | ¹H NMR (400 MHz, DMSO-d₆) δ 8.56 (s, 1H), 8.30 (s, 1H), 7.84 (d, J = 8.6 Hz, 2H), 7.56-7.41 (m, 1H), 7.36 (d, J = 8.6 Hz, 2H), 7.16 (t, J = 8.1 Hz, 2H), 5.30 (s, 2H), 4.97 (s, 2H), 2.25 (s, 3H) | 407.2 |
| | | 1-(4-(4-(2,6-difluorobenzyl)-5-oxo- | | | |
| | | 4,5-dihydro-1H-1,2,4-triazol-1- | | | |
| | | yl)benzyl)-3-methyl-1H-pyrazole- | | | |
| | | 4-carbonitrile | | | |
| Example 191 Method 53 | ++++ | 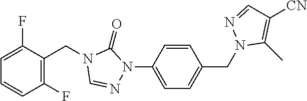 [SMILES: Cc1c(C#N)cnn1Cc1ccc(-n2ncn(Cc3c(F)cccc3F)c2=O)cc1] | white solid, yield: 25.3% | ¹H NMR (400 MHz, DMSO-d₆) δ 8.30 (s, 1H), 7.99 (s, 1H), 7.86-7.79 (m, 2H), 7.54-7.41 (m, 1H), 7.27 (d, J = 8.7 Hz, 2H), 7.22-7.08 (m, 2H), | 407.2 |
| | | 1-(4-(4-(2,6-difluorobenzyl)-5-oxo- | | 5.39 (s, 2H), 4.97 (s, 2H), | |
| | | 4,5-dihydro-1H-1,2,4-triazol-1- | | 2.40 (s, 3H). | |
| | | yl)benzyl)-5-methyl-1H-pyrazole- | | | |
| | | 4-carbonitrile | | | |
| Example 192 Method 53 | ++++ | 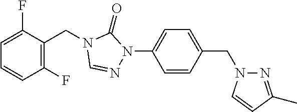 [SMILES: Cc1ccn(Cc2ccc(-n3ncn(Cc4c(F)cccc4F)c3=O)cc2)n1] | white solid, yield: 8% | ¹H NMR (400 MHz, DMSO-d₆) δ 8.29 (s, 1H), 7.81 (d, J = 8.6 Hz, 2H), 7.67 (d, J = 2.1 Hz, 1H), 7.52-7.44 (m, 1H), 7.35 (d, J = 1.5 Hz, 1H), 7.29 (d, J = 8.6 Hz, 1H), 7.19-7.12 (m, | 382.2 |
| | | 4-(2,6-difluorobenzyl)-2-(4-((3- | | 2H), 6.02 (d, J = 2.1 Hz, | |
| | | methyl-1H-pyrazol-1-yl)methyl) | | 1H), 5.21 (s, 2H), 4.96 (s, | |
| | | phenyl)-2,4-dihydro-3H-1,2,4- | | 2H), 2.13 (s, 3H). | |
| | | triazol-3-one | | | |
| Example 193 Method 53 | ++ | 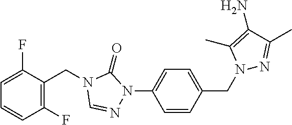 [SMILES: Cc1nn(Cc2ccc(-n3ncn(Cc4c(F)cccc4F)c3=O)cc2)c(C)c1N] | white solid, yield: 44% | ¹H NMR (400 MHz, DMSO-d₆) δ 8.27 (s, 1H), 7.75 (d, J = 8.6 Hz, 2H), 7.53-7.43 (m, 1H), 7.34 (d, J = 8.6 Hz, 2H), 7.16 (t, J = 8.1 Hz, 2H), 4.96 (s, 2H), 3.89 (s, 2H), 1.94 (s, 6H). | 411.2 |
| | | 2-(4-((4-amino-3,5-dimethyl-1H- | | | |
| | | pyrazol-1-yl)methyl)phenyl)-4- | | | |
| | | (2,6-difluorobenzyl)-2,4-dihydro- | | | |
| | | 3H-1,2,4-triazol-3-one | | | |
| Example 194 Method 53 | ++ | 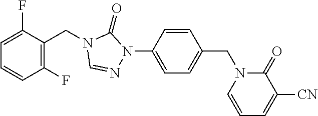 [SMILES: N#Cc1cccn(Cc2ccc(-n3ncn(Cc4c(F)cccc4F)c3=O)cc2)c1=O] | white solid, yield: 7% | ¹H NMR (400 MHz, DMSO-d₆) δ 8.29 (s, 1H), 8.26 (d, J = 5.6 Hz, 1H), 8.16 (d, J = 6.1 Hz, 1H), 7.84 (d, J = 8.3 Hz, 2H), 7.46 (d, J = 7.7 Hz, 1H), 7.41 (d, J = 8.3 Hz, 2H), | 420.1 |
| | | 1-(4-(4-(2,6-difluorobenzyl)-5-oxo- | | 7.15 (t, J = 7.9 Hz, 2H), | |
| | | 4,5-dihydro-1H-1,2,4-triazol-1- | | 6.45 (t, J = 6.9 Hz, 1H), | |
| | | yl)benzyl)-2-oxo-1,2-dihydro- | | 5.16 (s, 2H), 4.96 (s, 2H). | |
| | | pyridine-3-carbonitrile | | | |
| Example 195 Method 53 | +++ | 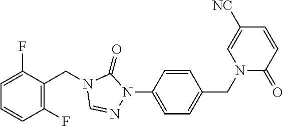 [SMILES: N#Cc1ccc(=O)n(Cc2ccc(-n3ncn(Cc4c(F)cccc4F)c3=O)cc2)c1] | white solid, yield: 31% | ¹H NMR (400 MHz, DMSO-d₆) δ 8.80 (d, J = 2.4 Hz, 1H), 8.29 (s, 1H), 7.83 (d, J = 8.7 Hz, 2H), 7.69 (dd, J = 9.5, 2.5 Hz, 1H), 7.52-7.45 (m, 1H), 7.43 (d, J = 8.7 Hz, 2H), 7.15 (t, J = 8.1 Hz, 2H), | 420.1 |
| | | 1-(4-(4-(2,6-difluorobenzyl)-5-oxo- | | 6.51 (d, J = 9.5 Hz, 1H), | |
| | | 4,5-dihydro-1H-1,2,4-triazol-1- | | 5.10 (s, 2H), 4.97 (s, 2H). | |
| | | yl)benzyl)-6-oxo-1,6-dihydro- | | | |
| | | pyridine-3-carbonitrile | | | |
| Example 196 Method 53 | ++++ | 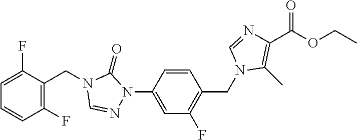 [SMILES: CCOC(=O)c1ncn(Cc2ccc(-n3ncn(Cc4c(F)cccc4F)c3=O)cc2F)c1C] | white solid, yield: 33.8% | ¹H NMR (400 MHz, CDCl₃) δ 7.88-7.79 (m, 3H), 7.59 (s, 1H), 7.37 (s, 1H), 7.26-723 (m, 2H), 7.01-6.98 (m, 1H), 5.57 (s, 2H), 4.97 (s, 2H), 4.34 (q, J = 7.1 Hz, 2H), 2.59 (s, 3H), 1.36 (t, J = 7.1 Hz, 3H). | 472.4 |
| | | ethyl | | | |
| | | 1-(4-(4-(2,6-difluorobenzyl)-5-oxo- | | | |
| | | 4,5-dihydro-1H-1,2,4-triazol-1- | | | |
| | | yl)-2-fluorobenzyl)-5-methyl-1H- | | | |
| | | imidazole-4-carboxylate | | | |
| Example 197 Method 53 | ++++ | 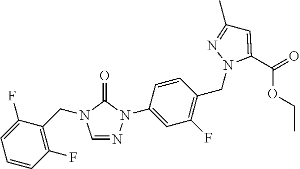 [SMILES: CCOC(=O)c1cc(C)nn1Cc1ccc(-n2ncn(Cc3c(F)cccc3F)c2=O)cc1F] | white solid, yield: 1.41% | ¹H NMR (400 MHz, CDCl₃) δ 7.77 (dd, J = 11.7, 2.0 Hz, 1H), 7.67 (dd, J = 8.5, 1.7 Hz, 1H), 7.54 (s, 1H), 7.40-7.30 (m, 1H), 7.01-6.93 (m, 2H), 6.90 (t, J = 8.3 Hz, 1H), 6.68 (s, 1H), 5.75 (s, 2H), 4.95 (s, | 472.4 |
| | | ethyl | | 2H), 4.28 (q, J = 7.1 Hz, | |
| | | 1-(4-(4-(2,6-difluorobenzyl)-5-oxo- | | 2H), 2.29 (s, 3H), 1.31 | |
| | | 4,5-dihydro-1H-1,2,4-triazol-1- | | (t, J = 7.1 Hz, 3H). | |
| | | yl)-2-fluorobenzyl)-3-methyl-1H- | | | |
| | | pyrazole-5-carboxylate | | | |
| Example 198 Method 53 | +++ | 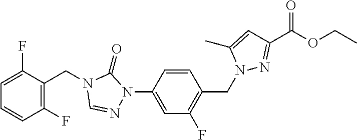 [SMILES: CCOC(=O)c1cc(C)n(Cc2ccc(-n3ncn(Cc4c(F)cccc4F)c3=O)cc2F)n1] | white solid, yield: 1.69% | ¹H NMR (400 MHz, CDCl₃) δ 7.84 (dd, J = 11.8, 2.0 Hz, 1H), 7.70 (dd, J = 8.5, 1.8 Hz, 1H), 7.56 (s, 1H), 7.41- 7.30 (m, 1H), 6.97 (dd, J = 13.9, 5.6 Hz, 3H), 6.60 (s, 1H), 5.40 (s, 2H), 4.96 (s, 2H), 4.40 | 472.4 |
| | | ethyl | | (q, J = 7.1 Hz, 2H), | |
| | | 1-(4-(4-(2,6-difluorobenzyl)-5-oxo- | | 2.21 (s, 3H), 1.39 (t, | |
| | | 4,5-dihydro-1H-1,2,4-triazol-1- | | J = 7.1 Hz, 3H). | |
| | | yl)-2-fluorobenzyl)-5-methyl-1H- | | | |
| | | pyrazole-3-carboxylate | | | |
| Example 199 Method 53 | +++ | 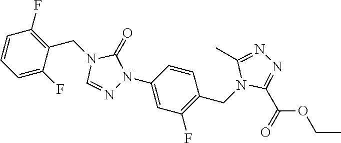 [SMILES: CCOC(=O)c1nnc(C)n1Cc1ccc(-n2ncn(Cc3c(F)cccc3F)c2=O)cc1F] | white solid, yield: 4.8% | ¹H NMR (400 MHz, CDCl₃) δ 7.82 (dd, J = 11.7, 1.4 Hz, 1H), 7.75 (d, J = 8.5 Hz, 1H), 7.57 (s, 1H), 7.36 (dd, J = 11.4, 4.6 Hz, 1H), 7.18-7.15 (m, 1H), 6.98-6.96 (m, 2H), 5.80 (s, 2H), 4.96 (s, 2H), 4.49-4.41 (m, 2H), 2.42 | 473.2 |
| | | ethyl | | (s, 3H), 1.43-1.40 (m, 3H). | |
| | | 1-(4-(4-(2,6-difluorobenzyl)-5-oxo- | | | |
| | | 4,5-dihydro-1H-1,2,4-triazol-1- | | | |
| | | yl)-2-fluorobenzyl)-5-methyl-1H- | | | |
| | | 1,2,4-triazole-3-carboxylate | | | |
| Example 200 Method 53 | +++ | 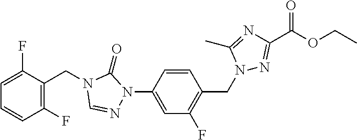 [SMILES: CCOC(=O)c1nc(C)n(Cc2ccc(-n3ncn(Cc4c(F)cccc4F)c3=O)cc2F)n1] | white solid, yield: 33.7% | ¹HNMR (400 MHz, CDCl₃) δ 7.87-7.84 (m, 1H), 7.77-7.73 (m, 1H), 7.57 (s, 1H), 7.36-7.32 (m, 1H), 7.23-7.19 (m, 1H), 6.98-6.96 (m, 2H), 5.37 (s, 2H), 4.94 (s, 2H), 4.46- 4.42 (m, 2H), 2.47 (s, 3H), 1.39-1.37 (m, 3H). | 473.2 |
| | | ethyl | | | |
| | | 1-(4-(4-(2,6-difluorobenzyl)-5-oxo- | | | |
| | | 4,5-dihydro-1H-1,2,4-triazol-1- | | | |
| | | yl)-2-fluorobenzyl)-3-methyl-1H- | | | |
| | | 1,2,4-triazole-5-carboxylate | | | |
| Example 201 Method 53 | +++ | 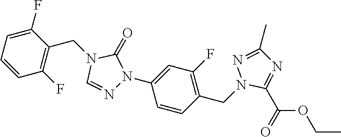 [SMILES: CCOC(=O)c1nc(C)nn1Cc1ccc(-n2ncn(Cc3c(F)cccc3F)c2=O)cc1F] | white solid, yield: 39.8% | ¹H NMR (400 MHz, CDCl₃) δ 7.82 (dd, J = 11.7, 1.4 Hz, 1H), 7.75 (d, J = 8.5 Hz, 1H), 7.57 (s, 1H), 7.36 (dd, J = 11.4, 4.6 Hz, 1H), 7.18-7.15 (m, 1H), 6.98-6.96 m, 2H), 5.80 (s, 2H), 4.96 (s, 2H), | 473.2 |
| | | 2-(1-(4-(4-(2,6-difluorobenzyl)-5- | | 4.49-4.41 (m, 2H), 2.42 | |
| | | oxo-4,5-dihydro-1H-1,2,4-triazol- | | (s, 3H), 1.43-1.40 (m, 3H). | |
| | | 1-yl)-2-fluorobenzyl)-2-methyl- | | | |
| | | 1H-imidazol-4-yl)acetonitrile | | | |
| Example 202 Method 53 | ++++ | 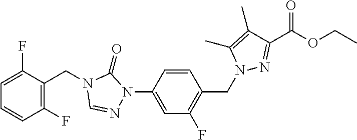 [SMILES: CCOC(=O)c1nn(Cc2ccc(-n3ncn(Cc4c(F)cccc4F)c3=O)cc2F)c(C)c1C] | off-white solid, yield: 4.9% | ¹H NMR (400 MHz, CDCl₃) δ 7.83-7.80 (m, 1H), 7.67-7.65 (m, 1H), 7.56 (s, 1H), 7.35-7.33 (m, 1H), 6.95-6.93 m, 3H), 5.38 (s, 2H), 4.94 (s, 2H), 4.39-4.37 (m, 2H), 2.19 (s, 3H), 2.07 (s, 3H), 1.39-1.37 (m, 3H). | 486.2 |
| | | ethyl | | | |
| | | 1-(4-(4-(2,6-difluorobenzyl)-5-oxo- | | | |
| | | 4,5-dihydro-1H-1,2,4-triazol-1- | | | |
| | | yl)-2-fluorobenzyl)-4,5-dimethyl- | | | |
| | | 1H-pyrazole-3-carboxylate | | | |
| Example 203 Method 53 | +++ | 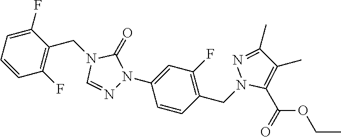 [SMILES: CCOC(=O)c1c(C)c(C)nn1Cc1ccc(-n2ncn(Cc3c(F)cccc3F)c2=O)cc1F] | off-white solid, yield: 4.1% | ¹H NMR (400 MHz, CDCl₃) δ 7.77-7.74 (m, 1H), 7.66-7.61 (m, 1H), 7.54 (s, 1H), 7.34-7.32 (m, 1H), 7.00-6.91 (m, 2H), 6.83-6.79 (m, 1H), 5.70 (s, 2H), 4.94 (s, 2H), 4.26- 4.24 (m, 2H), 2.21 (s, 3H), | 486.2 |
| | | ethyl | | 2.19 (s, 3H), 1.29-1.27 (m, | |
| | | 1-(4-(4-(2,6-difluorobenzyl)-5-oxo- | | 3H). | |
| | | 4,5-dihydro-1H-1,2,4-triazol-1- | | | |
| | | yl)-2-fluorobenzyl)-3,4-dimethyl- | | | |
| | | 1H-pyrazole-5-carboxylate | | | |
| Example 204 Method 53 | ++++ | 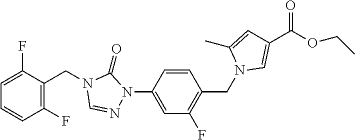 [SMILES: CCOC(=O)c1cc(C)n(Cc2ccc(-n3ncn(Cc4c(F)cccc4F)c3=O)cc2F)c1] | white solid, yield: 3.1% | ¹H NMR (400 MHz, DMSO-d₆) δ 8.34 (s, 1H), 7.75 (d, J = 12.1 Hz, 1H), 7.68 (d, J = 8.4 Hz, 1H), 7.52-7.44 (m, 2H), 7.41 (d, J = 1.2 Hz, 1H), 7.15 (t, J = 8.0 Hz, 1H), 7.05 (t, J = 8.5 Hz, 1H), 6.20 (s, 1H), 5.18 (s, 2H), 4.97 (s, 2H), | 471.1 |
| | | ethyl | | 4.13 (q, J = 7.1 Hz, 2H), | |
| | | 1-(4-(4-(2,6-difluorobenzyl)-5-oxo- | | 2.10 (s, 3H), 1.22 (t, J = | |
| | | 4,5-dihydro-1H-1,2,4-triazol-1- | | 7.1 Hz, 3H). | |
| | | yl)-2-fluorobenzyl)-5-methyl-1H- | | | |
| | | pyrrole-3-carboxylate | | | |
| Example 205 Method 53 | ++++ | 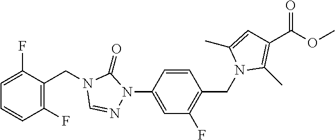 [SMILES: COC(=O)c1cc(C)n(Cc2ccc(-n3ncn(Cc4c(F)cccc4F)c3=O)cc2F)c1C] | white solid, yield: 2.8% | ¹H NMR (400 MHz, CD₃OD) δ 8.00 (s, 1H), 7.78 (dd, J = 12.0, 2.0 Hz, 1H), 7.65 (dd, J = 8.5, 1.8 Hz, 1H), 7.49-7.38 (m, 1H), 7.05 (t, J = 8.1 Hz, 2H), 6.50 (t, J = 8.4 Hz, 1H), 6.29 (s, 1H), 5.17 (s, 2H), 5.02 (s, 2H), 3.76 (s, 3H), 2.42 (s, | 471.1 |
| | | methyl | | 3H), 2.12 (s, 3H) | |
| | | 1-(4-(4-(2,6-difluorobenzyl)-5-oxo- | | | |
| | | 4,5-dihydro-1H-1,2,4-triazol-1- | | | |
| | | yl)-2-fluorobenzyl)-2,5-dimethyl- | | | |
| | | 1H-pyrrole-3-carboxylate | | | |
| Example 206 Method 53 | +++ | 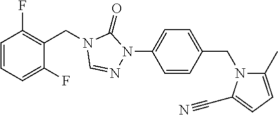 [SMILES: Cc1ccc(C#N)n1Cc1ccc(-n2ncn(Cc3c(F)cccc3F)c2=O)cc1] | white solid, yield: 20% | ¹H NMR (400 MHz, DMSO-d₆) δ 8.30 (s, 1H), 7.84 (d, J = 8.6 Hz, 2H), 7.52-7.42 (m, 2H), 7.20- 7.15 (m, 1H), 7.12-7.08 (m, 2H), 6.94 (d, J = 3.9 Hz, 1H), 6.07 (d, J = 3.9 Hz, 1H), | 406.1 |
| | | 1-(4-(4-(2,6-difluorobenzyl)-5-oxo- | | 5.26 (s, 2H), 4.97 (s, 2H), | |
| | | 4,5-dihydro-1H-1,2,4-triazol-1- | | 2.20 (s, 3H). | |
| | | yl)benzyl)-5-methyl-1H-pyrrole-2- | | | |
| | | carbonitrile | | | |
| Example 207 Method 53 | ++++ | 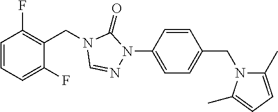 [SMILES: Cc1ccc(C)n1Cc1ccc(-n2ncn(Cc3c(F)cccc3F)c2=O)cc1] | white solid, yield: 2% | ¹H NMR (400 MHz, CDCl₃) δ 7.98 (s, 1H), 7.74-7.65 (m, 2H), 7.53- 7.39 (m, 2H), 7.22 (d, J = 6.8 Hz, 2H), 7.12-7.02 (m, 3H), 5.47 (s, 2H), 5.04 (s, 2H), 2.12 (d, J = 14.1 Hz, | 395.1 |
| | | 4-(2,6-difluorobenzyl)-2-(4-((2,5- | | 6H). | |
| | | dimethyl-1H-pyrrol-1-yl)methyl) | | | |
| | | phenyl)-2,4-dihydro-3H-1,2,4- | | | |
| | | triazol-3-one | | | |
| Example 208 Method 53 | ++++ | 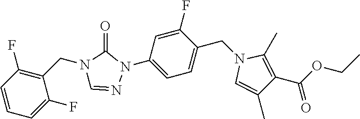 [SMILES: CCOC(=O)c1c(C)cn(Cc2ccc(-n3ncn(Cc4c(F)cccc4F)c3=O)cc2F)c1C] | white solid, yield: 32.8% | ¹H NMR (400 MHz, CDCl₃) δ 7.86-7.83 (m, 1H), 7.72-7.69 (m, 1H), 7.57 (s, 1H), 7.38-7.34 (m, 1H), 6.99-6.97 (m, 2H), 6.79-6.77 (m, 1H), 6.34 (s, 1H), 4.99-4.96 (m, 4H),\ 4.29-4.26 (m, 2H), 2.44 | 485.2 |
| | | 1-(4-(4-(2,6-difluorobenzyl)-5-oxo- | | (s, 3H), 2.22 (s, 3H), 1.35- | |
| | | 4,5-dihydro-1H-1,2,4-triazol-1- | | 1.33 (m, 3H). | |
| | | yl)-2-fluorobenzyl)-2,4-dimethyl- | | | |
| | | 1H-pyrrole-3-carboxylate | | | |
| Example 209 Method 53 | ++++ | 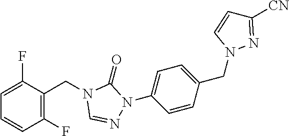 [SMILES: N#Cc1ccn(Cc2ccc(-n3ncn(Cc4c(F)cccc4F)c3=O)cc2)n1] | white solid, yield: 53.2% | ¹H NMR (400 MHz, CDCl₃) δ 8.03-7.98 (m, 2H), 7.59 (s, 1H), 7.39- 7.34 (m, 2H), 7.30 (d, J = 8.7 Hz, 2H), 7.02-6.95 (m, 2H), 6.67 (d, J = 2.4 Hz, 1H), 5.34 (s, 2H), 4.97 (s, 2H). | 393.2 |
| | | 1-(4-(4-(2,6-difluorobenzyl)-5-oxo- | | | |
| | | 4,5-dihydro-1H-1,2,4-triazol-1- | | | |
| | | yl)benzyl)-1H-pyrazole-3- | | | |
| | | carbonitrile | | | |
| Example 210 Method 53 | +++ | 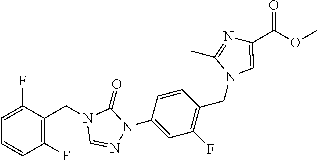 [SMILES: COC(=O)c1cn(Cc2ccc(-n3ncn(Cc4c(F)cccc4F)c3=O)cc2F)c(C)n1] | white solid, yield: 69.6% | ¹H NMR (400 MHz, CDCl₃) δ 7.93- 7.82 (m, 2H), 7.60 (s, 1H), 7.55 (s, 1H), 7.41-7.35 (m, 1H), 7.10 (m, 1H), 7.01-6.97 (m, 2H), 5.11 (s, 2H), 4.97 (s, 2H), 3.89 (s, 3H), 2.56 (s, 3H). | 458.1 |
| | | methyl | | | |
| | | 1-(4-(4-(2,6-difluorobenzyl)-5-oxo- | | | |
| | | 4,5-dihydro-1H-1,2,4-triazol-1- | | | |
| | | yl)-2-fluorobenzyl)-2-methyl-1H- | | | |
| | | imidazole-4-carboxylate | | | |
| Example 211 Method 53 | +++ | 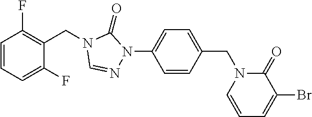 [SMILES: O=c1c(Br)cccn1Cc1ccc(-n2ncn(Cc3c(F)cccc3F)c2=O)cc1] | white solid, yield: | ¹H NMR (400 MHz, DMSO-d₆) δ 8.30 (s, 1H), 7.93 (d, J = 7.0 Hz, 2H), 7.83 (d, J = 8.6 Hz, 2H), 7.48 (t, J = 8.2 Hz, 1H), 7.40 (d, J = 8.6 Hz, 2H), 7.16 (t, J = 8.0 Hz, 2H), | 473.0, 475.0 |
| | | 3-bromo-1-(4-(4-(2,6-difluoro- | | 6.23 (t, J = 7.0 Hz, 1H), | |
| | | benzyl)-5-oxo-4,5-dihydro-1H-1,2,4- | | 5.15 (s, 2H), 4.97 (s, 2H). | |
| | | triazol-1-yl)benzyl)pyridin-2(1H)- | | | |
| | | one | | | |

### Example 212: Method 54

### 4-[(2,6-difluorophenyl)methyl]-2-(3-fluoro-4-{[4-methyl-2-(pyrrolidin-1-ylmethyl)-1,3-thiazol-5-yl]oxy}phenyl)-1,2,4-triazol-3-one

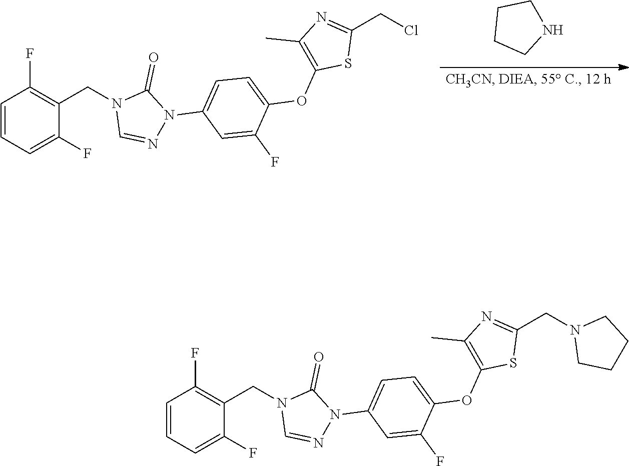

A mixture of 2-(4-{[2-(chloromethyl)-4-methyl-1,3-thiazol-5-yl]oxy}-3-fluorophenyl)-4-[(2,6-difluorophenyl)methyl]-1,2,4-triazol-3-one(100 mg, 0.2142 mmol), pyrrolidine(15.23 mg, 0.2142 mmol) and DIEA(55.37 mg, 0.4284 mmol) in CH₃CN(10 mL) was stirred at 55° C. for 12 h. The reaction mixture was stirred at 110° C. for 2 h. Water (20 mL) was added to the mixture, and the mixture was extracted with EtOAc (30 mL×3). The residue was purified by flash with the following conditions (PE:EtOAc=1:1) to afford 4-[(2,6-difluorophenyl)methyl]-2-(3-fluoro-4-{[4-methyl-2-(pyrrolidin-1-ylmethyl)-1,3-thiazol-5-yl]oxy}phenyl)-1,2,4-triazol-3-one (54 mg, 49.8% yield) as an off-white solid. MS (m/z): 502.1 [M+H]⁺. ¹H NMR (400 MHz, CDCl₃) δ 7.93-7.84 (m, 1H), 7.74-7.67 (m, 1H), 7.57 (s, 1H), 7.41-7.31 (m, 1H), 6.97 (dd, J=8.4, 7.5 Hz, 3H), 4.96 (s, 2H), 4.21-3.67 (m, 2H), 2.27 (s, 3H), 1.96-1.78 (m, 3H), 1.63-1.48 (m, 5H).

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 212 Method 54 | ++++ | 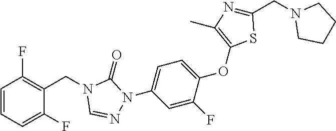 [SMILES: Cc1nc(CN2CCCC2)sc1Oc1ccc(-n2ncn(Cc3c(F)cccc3F)c2=O)cc1F] | off-white solid, yield: 49.8% | ¹H NMR (400 MHz, CDCl₃) δ 7.93-7.84 (m, 1H), 7.74-7.67 (m, 1H), 7.57 (s, 1H), 7.41- 7.31 (m, 1H), 6.97 (dd, J = 8.4, 7.5 Hz, 3H), 4.96 (s, 2H), 4.21-3.67 (m, 2H), 2.27 (s, 3H), 1.96-1.78 (m, 3H), 1.63-1.48 (m, 5H). | 502.1 |
| | | 4-(2,6-difluorobenzyl)-2-(3-fluoro- | | | |
| | | 4-((4-methyl-2-(pyrrolidin-1- | | | |
| | | ylmethyl)thiazol-5-yl)oxy)phenyl)- | | | |
| | | 2,4-dihydro-3H-1,2,4-triazol- | | | |
| | | 3-one | | | |

### Example 213: Method 55

### 2-(4-((2,4-dimethyl-1H-imidazol-1-yl)methyl)-3-fluorophenyl)-5-phenyl-2,5,6,7-tetrahydr o-3H-pyrrolo[2,1-c][1,2,4]triazol-3-one

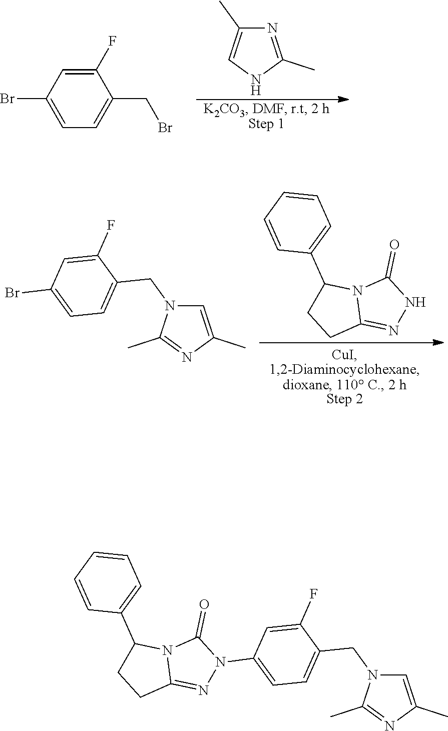

### Step 1: 1-(4-bromo-2-fluorobenzyl)-2,4-dimethyl-1H-imidazole

To a stirred solution of 4-bromo-1-(bromomethyl)-2-fluorobenzene (200 mg, 0.7522 mmol) and 2,4-dimethyl-1H-imidazole (72.22 mg, 0.7522 mmol) in DMF (4 mL) was added K₂CO₃(311.4 mg, 2.257 mmol) at rt. The resulted mixture was stirred for additional 2 h at rt. The resulted mixture was diluted with water (20 mL). The resulted mixture was extracted with EtOAc (3×20 mL). The organic layers were combined, the solvent was removed under vacuum and the crude was purified through silica gel chromatography (PE/EA=1:1) to give 1-(4-bromo-2-fluorobenzyl)-2,4-dimethyl-1H-imidazole. MS (m/z): 283.02 [M+H]⁺.

### Step 2: 2-(4-((2,4-dimethyl-1H-imidazol-1-yl)methyl)-3-fluorophenyl)-5-phenyl-2,5,6,7-tetrahydro-3H-pyrrolo[2,1-c][1,2,4]triazol-3-one

To a stirred solution of 1-(4-bromo-2-fluorobenzyl)-2,4-dimethyl-1H-imidazole (100 mg, 0.3545 mmol) and 5-phenyl-2,5,6,7-tetrahydro-3H-pyrrolo[2,1-c][1,2,4]triazol-3-one (71.30 mg, 0.3545 mmol) in dioxane (4 mL) were added copper(I) iodide (67.36 mg, 0.3545 mmol) and 1,2-Diaminocyclohexane (50.39 mg, 0.3545 mmol) at rt. The resulted mixture was stirred for additional 2 h at 110° C. under an atmosphere of nitrogen. The mixture was allowed to cool down to rt. The resulted mixture was diluted with water (20 mL). The resulted mixture was extracted with EtOAc (3×20 mL). The combined organic layers were washed with brine (20 mL). The residue was purified by reverse phase flash chromatography with the following conditions: column: Spherical C18 40-60 um, 40 g; Mobile phase B: ACN; Flow rate: 40 mL/min; Gradient: 30% B-60% B in 20 min; Detector: 254 nm. The fractions containing the desired product were collected at 55% B and concentrated under reduced pressure to afford the titled compound 2-(4-((2,4-dimethyl-1H-imidazol-1-yl)methyl)-3-fluorophenyl)-5-phenyl-2,5,6,7-tetrahydro-3H-pyrrolo[2,1-c][1,2,4]triazol-3-one (10 mg, 6.99%) yield as an off-white solid. LC-MS (m/z): 404.2 [M+H]⁺. ¹H NMR (400 MHz, CDCl₃) δ 7.85 (d, J=12.0 Hz, 1H), 7.79 (d, J=8.3 Hz, 1H), 7.38 (d, J=7.0 Hz, 2H), 7.34 (d, J=6.7 Hz, 1H), 7.28-7.23 (m, 2H), 7.09 (t, J=7.9 Hz, 1H), 6.58 (s, 1H), 5.27 (s, 1H), 5.01 (s, 2H), 3.13-2.99 (m, 2H), 2.93 (m, J=19.2, 11.8 Hz, 1H), 2.54 (s, 3H), 2.48 (s, 1H), 2.23 (s, 3H).

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 213 Method 55 | ++++ | 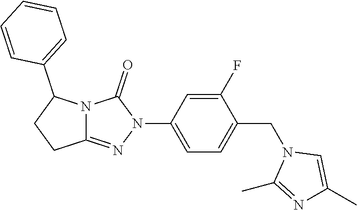 [SMILES: Cc1cn(Cc2ccc(-n3nc4n(c3=O)C(c3ccccc3)CC4)cc2F)c(C)n1] | off-white solid, yield: 7% | ¹H NMR (400 MHz, CDCl₃) δ 7.85 (d, J = 12.0 Hz, 1H), 7.79 (d, J = 8.3 Hz, 1H), 7.38 (d, J = 7.0 Hz, 2H), 7.34 (d, J = 6.7 Hz, 1H), 7.28-7.23 (m, 2H) , 7.09 (t, J = 7.9 Hz, 1H), 6.58 (s, 1H), 5.27 (s, 1H), 5.01 (s, 2H), 3.13-2.99 (m, 2H), 2.93 (m, J = 19.2, 11.8 Hz, 1H), 2.54 (s, 3H), 2.48 (s, 1H), 2.23 (s, 3H). | 404.2 |
| | | 2-(4-((2,4-dimethyl-1H-imidazo1- | | | |
| | | 1-yl)methyl)-3-fluorophenyl)- | | | |
| | | 5-phenyl-2,5,6,7-tetrahydro-3H- | | | |
| | | pyrrolo[2,1-c][1,2,4]triazol-3-one | | | |

### Example 214: Method 56

### N-(3,5-difluorobenzyl)-1-(4-(3,5-dimethyl-1H-1,2,4-triazol-1-yl)-5-fluoropyrimidin-2-yl)-N-hydroxypiperidine-4-carboxamide

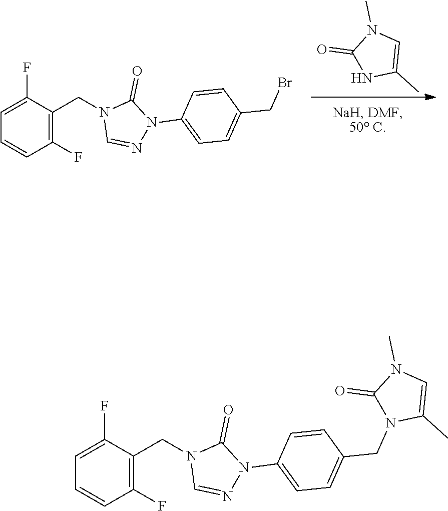

### 4-(2,6-difluorobenzyl)-2-(4-((3,5-dimethyl-2-oxo-2,3-dihydro-1H-imidazol-1-yl)methyl)ph enyl)-2,4-dihydro-3H-1,2,4-triazol-3-one

A stirred solution of 1,4-dimethyl-1,3-dihydro-2H-imidazol-2-one (106 mg, 0.95 mmol) and NaH (47 mg, 1.18 mmol) in DMF (15 mL) was stirred at 50° C. for 1h. Then the mixture was added 2-(4-(bromomethyl)phenyl)-4-(2,6-difluorobenzyl)-2,4-dihydro-3H-1,2,4-triazol-3-one (300 mg, 0.79 mmol) at 50° C. The resulted mixture was stirred for additional 2 h at 80° C. The mixture was allowed to cool down to rt. The resulted mixture was quenched by water (1 mL). The resulted mixture was filtered. The filtrate was diluted with EA (100 mL), washed with water (150 mL), dried over Na₂SO₄, filtered and the filtrate was concentrated under reduced pressure. The residue was purified by prep-HPLC to afford 4-(2,6-difluorobenzyl)-2-(4-((3,5-dimethyl-2-oxo-2,3-dihydro-1H-imidazol-1-yl)methyl)pheny 1)-2,4-dihydro-3H-1,2,4-triazol-3-one (5 mg, 1.54% yield) as a white solid. MS (m/z): 412.2 [M+H]⁺. ¹H NMR (400 MHz, CD₃OD) δ 7.97 (s, 1H), 7.84 (d, J=8.7 Hz, 2H), 7.43 (m, 1H), 7.25 (d, J=8.8 Hz, 2H), 7.05 (t, J=8.1 Hz, 2H), 6.20 (d, J=1.3 Hz, 1H), 5.02 (s, 2H), 4.87 (s, 2H), 3.24 (s, 3H), 1.94 (d, J=1.3 Hz, 3H).

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 214 Method 56 | ++ | 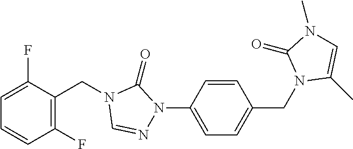 [SMILES: Cc1cn(C)c(=O)n1Cc1ccc(-n2ncn(Cc3c(F)cccc3F)c2=O)cc1] | white solid, yield: 1.54% | ¹H NMR (400 MHz, CD₃OD) δ 7.97 (s, 1H), 7.84 (d, J = 8.7 Hz, 2H), 7.43 (m, 1H), 7.25 (d, J = 8.8 Hz, 2H), 7.05 (t, J = 8.1 Hz, 2H), 6.20 (d, J = 1.3 Hz, 1H), 5.02 (s, 2H), 4.87 (s, 2H), 3.24 (s, 3H), 1.94 (d, J = 1.3 Hz, 3H). | 412.2 |
| | | 4-(2,6-difluorobenzyl)-2-(4-((3,5- | | | |
| | | dimethyl-2-oxo-2,3-dihydro-1H- | | | |
| | | imidazol-1-yl)methyl)phenyl)- | | | |
| | | 2,4-dihydro-3H-1,2,4-triazol-3- | | | |
| | | one | | | |

### Example 215: Method 57

### Ethyl 1-(4-(4-(2,6-difluorobenzyl)-5-oxo-4,5-dihydro-1H-1,2,4-triazol-1-yl)-2-fluorobenzyl)-2,5-dimethyl-1H-imidazole-4-carboxylate

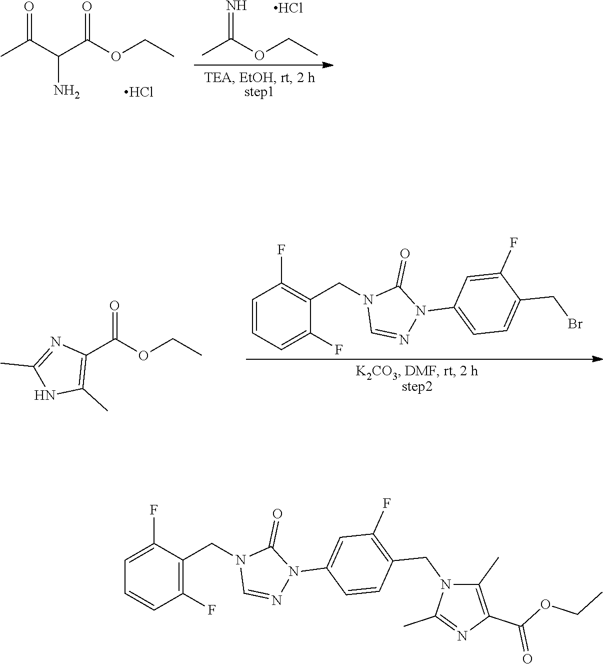

### Step 1: ethyl 2,5-dimethyl-1H-imidazole-4-carboxylate

TEA (3.89 g, 0.0385 mol) was added to a solution of ethyl 2-amino-3-oxobutanoate hydrochloride (2.0 g, 0.011 mol), ethyl ethanecarboximidate hydrochloride(3.4 g, 0.0275 mol) in EtOH (50 mL). The mixture was stirred at rt for 16h, The resulted mixture was diluted with water (20 mL). The resulted mixture was extracted with EtOAc (3×20 mL). The organic layers were combined, and the solvent was removed under vacuum and the crude was purified through silica gel chromatography (PE/EA=1:1) to give 1-(4-bromo-2-fluorobenzyl)-2,4-dimethyl-1H-imidazole as yellow solid (900 mg, 49.1% yield). LCMS (m/z): 169.1 [M+H]⁺, RT: 0.415 min.

### Step 2: ethyl 1-(4-(4-(2,6-difluorobenzyl)-5-oxo-4,5-dihydro-1H-1,2,4-triazol-1-yl)-2-fluorobenzyl)-2,5-dimethyl-1H-imidazole-4-carboxylate

To a stirred solution of ethyl 2,5-dimethyl-1H-imidazole-4-carboxylate (0.84 g, 5.02 mmol) and 2-[4-(bromomethyl)-3-fluorophenyl]-4-[(2,6-difluorophenyl)methyl]-1,2,4-triazol-3-one (2000 mg, 5.02 mmol) in DMF (40 mL) was added K₂CO₃ (2082 mg, 15.07 mmol) at rt. The resulted mixture was stirred for additional 16 h at 25° C. The resulted mixture was diluted with water (50 mL). The resulted mixture was extracted with EtOAc (3×30 mL). The combined organic layers were washed with brine (20 mL), dried over anhydrous Na₂SO₄, and after filtration, the filtrate was concentrated under reduced pressure. The residue was purified by silica gel column chromatography, eluted with (PE:EtOAc) (1:1) to afford 4-(2,6-difluorobenzyl)-2-(4-nitrophenyl)-2,4-dihydro-3H-1,2,4-triazol-3-one (1200 mg, 49.2% yield) as a white solid. LCMS (m/z): 486.1 [M+H]⁺, RT: 1.091 min. ¹H NMR (400 MHz, CDCl₃) δ 7.86 (d, J=12.0 Hz, 1H), 7.76 (d, J=7.8 Hz, 1H), 7.58 (s, 1H), 7.39-7.35 (m, 1H), 6.99 (d, J=7.8 Hz, 2H), 6.88 (s, 1H), 5.60 (s, 2H), 4.96 (s, 2H), 4.31 (d, J=7.1 Hz, 2H), 2.62 (s, 6H), 1.33 (t, J=7.1 Hz, 3H).

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 215 Method 57 | ++++ | 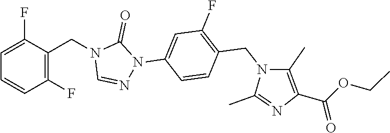 [SMILES: CCOC(=O)c1nc(C)n(Cc2ccc(-n3ncn(Cc4c(F)cccc4F)c3=O)cc2F)c1C] | white solid, yield: 49.2% | ¹H NMR (400 MHz, CDCl₃) δ 7.86 (d, J = 12.0 Hz, 1H), 7.76 (d, J = 7.8 Hz, 1H), 7.58 (s, 1H), 7.39-7.35 (m, 1H), 6.99 (d, J = 7.8 Hz, 2H), 6.88 (s, 1H), 5.60 (s, 2H), 4.96 (s, 2H), | 486.1 |
| | | ethyl | | 4.31 (d, J = 7.1 Hz, 2H), | |
| | | 1-(4-(4-(2,6-difluorobenzyl)-5- | | 2.62 (s, 6H), 1.33 | |
| | | oxo-4,5-dihydro-1H-1,2,4-triazol- | | (t, J = 7.1 Hz, 3H). | |
| | | 1-yl)-2-fluorobenzyl)-2,5-dimethyl- | | | |
| | | 1H-imidazole-4-carboxylate | | | |

### Example 216: Method 58

### 1-(4-(4-(2,6-difluorobenzyl)-5-oxo-4,5-dihydro-1H-1,2,4-triazol-1-yl)-2-fluorobenzyl)-5-methyl-1H-pyrazole-3-carboxylic Acid

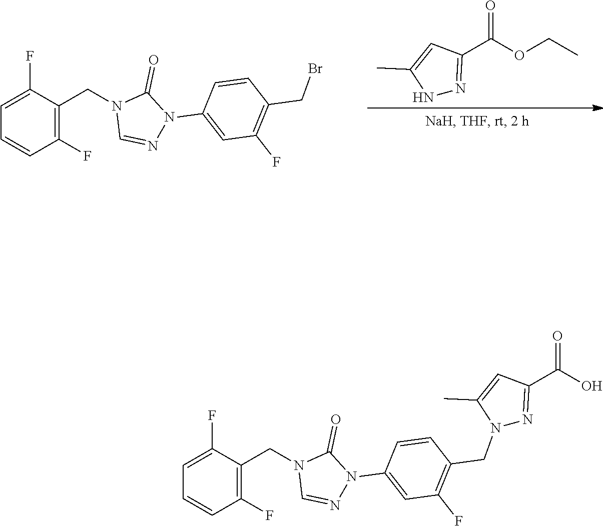 [SMILES: O=c1n(Cc2c(F)cccc2F)cnn1-c1ccc(CBr)c(F)c1 ; CCOC(=O)c1cc(C)[nH]n1 ; Cc1cc(C(=O)O)nn1Cc1ccc(-n2ncn(Cc3c(F)cccc3F)c2=O)cc1F]

A solution of 2-(4-(bromomethyl)-3-fluorophenyl)-4-(2,6-difluorobenzyl)-2,4-dihydro-3H-1,2,4-triazol-3-one (50 mg, 0.13 mmol) and NaH (8 mg, 0.19 mmol) in THF (5 mL) was stirred at rt for 30 min. Then the mixture was added ethyl 5-methyl-1H-pyrazole-3-carboxylate (23 mg, 0.15 mmol) at rt. The resulted mixture was stirred for additional 2 h at rt. The reaction was quenched by water (1 mL). The resulted mixture was diluted with water (50 mL). The resulted mixture was extracted with EtOAc (3×50 mL). The combined organic layers were washed with brine (100 mL), dried over anhydrous Na₂SO₄, and after filtration, the filtrate was concentrated under reduced pressure. The residue was purified by prep-HPLC to afford 1-(4-(4-(2,6-difluorobenzyl)-5-oxo-4,5-dihydro-1H-1,2,4-triazol-1-yl)-2-fluorobenzyl)-5-meth yl-1H-pyrazole-3-carboxylic acid (1 mg, 1.80% yield) as a white solid. MS (m/z): 444.4 [M+H]⁺. ¹H NMR (400 MHz, CDCl₃) δ 7.86 (dd, J=11.8, 2.0 Hz, 1H), 7.74 (d, J=8.3 Hz, 1H), 7.57 (s, 1H), 7.41-7.30 (m, 1H), 7.04 (t, J=8.4 Hz, 1H), 7.00-6.95 (m, 2H), 6.64 (s, 1H), 5.36 (s, 2H), 4.96 (s, 2H), 2.26 (s, 3H).

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 216 Method 58 | ++ | 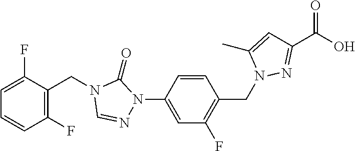 [SMILES: Cc1cc(C(=O)O)nn1Cc1ccc(-n2ncn(Cc3c(F)cccc3F)c2=O)cc1F] | white solid, yield: 1.8% | ¹H NMR (400 MHz, CDCl₃) δ 7.86 (dd, J = 11.8, 2.0 Hz, 1H), 7.74 (d, J = 8.3 Hz, 1H), 7.57 (s, 1H), 7.41-7.30 (m, 1H), 7.04 (t, J = 8.4 Hz, 1H), 7.00-6.95 (m, 2H), 6.64 (s, 1H), 5.36 (s, 2H), 4.96 (s, 2H), 2.26 (s, 3H). | 444.4 |
| | | 1-(4-(4-(2,6-difluorobenzyl)-5-oxo- | | | |
| | | 4,5-dihydro-1H-1,2,4-triazol- | | | |
| | | 1-yl)-2-fluorobenzyl)-5-methyl- | | | |
| | | 1H-pyrazole-3-carboxylic acid | | | |

### Example 217: Method 59

### 1-(4-(4-(2,6-difluorobenzyl)-5-oxo-4,5-dihydro-1H-1,2,4-triazol-1-yl)-2-fluorobenzyl)-2,5-dimethyl-1H-imidazole-4-carboxamide

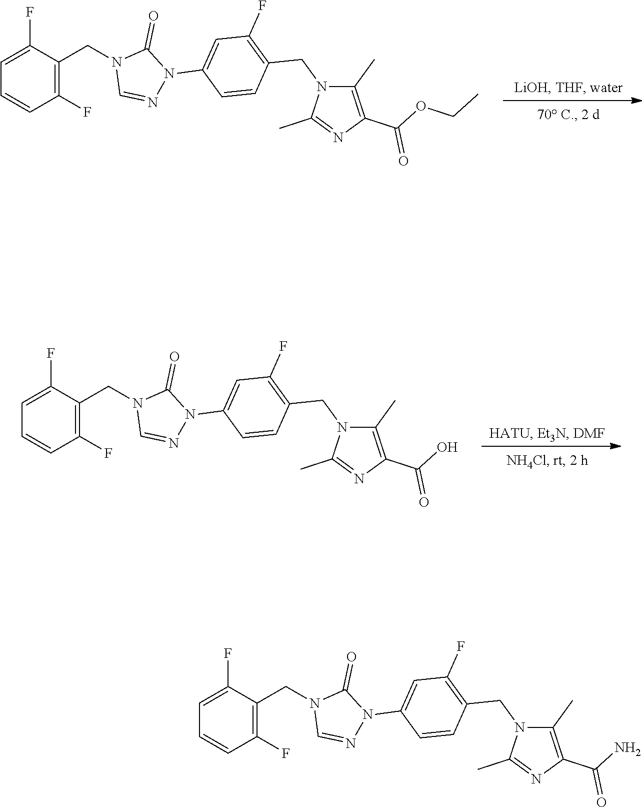

### Step 1: 1-(4-(4-(2,6-difluorobenzyl)-5-oxo-4,5-dihydro-1H-1,2,4-triazol-1-yl)-2-fluorobenzyl)-2,5-dimethyl-1H-imidazole-4-carboxylic acid

LiOH·H₂O (0.34 g, 8.24 mmol) was added to a solution of ethyl 1-[(4-{4-[(2,6-difluorophenyl)methyl]-5-oxo-1,2,4-triazol-1-yl}-2-fluorophenyl)methyl]-2,5-di methylimidazole-4-carboxylate(0.4 g, 0.82 mmol) in THF/water(1:1, 40 mL). The mixture was heated to 70° C. for 48h, and then cooled to room temperature. The solvent was removed under vacuum. The resulted mixture was diluted with water (20 mL), and adjusted to PH=3 with 1N HCl. The resulted mixture was extracted with EtOAc (3×20 mL). The organic layers were combined, and the solvent was removed under vacuum to give 1-(4-(4-(2,6-difluorobenzyl)-5-oxo-4,5-dihydro-1H-1,2,4-triazol-1-yl)-2-fluorobenzyl)-2,5-dim ethyl-1H-imidazole-4-carboxylic acid (360 mg, 95.5% yield). LCMS (m/z): 458.1 [M+H]⁺.

### Step 2: 1-(4-(4-(2,6-difluorobenzyl)-5-oxo-4,5-dihydro-1H-1,2,4-triazol-1-yl)-2-fluorobenzyl)-2,5-dimethyl-1H-imidazole-4-carboxamide

HATU (50 mg, 0.13 mmol) and triethylamine (27 mg, 0.087 mmol) were added to a solution of 1-[(4-{4-[(2,6-difluorophenyl)methyl]-5-oxo-1,2,4-triazol-1-yl}-2-fluorophenyl)methyl]-2,5-di methylimidazole-4-carboxylic acid (40 mg, 0.087 mmol) in DMF(10 mL). The mixture was stirred at rt for 30 min, then NH₄Cl (19 mg, 0.35 mmol) was added, and the mixture was stirred at rt for 2 h. The resulted mixture was diluted with water (20 mL). The resulted mixture was extracted with EtOAc (3×20 mL). The combined organic layers were washed with brine (20 mL), dried over anhydrous Na₂SO₄, and after filtration, the filtrate was concentrated under reduced pressure. The residue was purified by Prep-HPLC (column-Gemini —C18 150×21.2 mm, Sum; Mobile phase: ACN-H₂O (0.1% FA), 5%-20%) to afford 1-(4-(4-(2,6-difluorobenzyl)-5-oxo-4,5-dihydro-1H-1,2,4-triazol-1-yl)-2-fluorobenzyl)-2,5-dim ethyl-1H-imidazole-4-carboxamide (4.7 mg, 11.8% yield). LCMS (m/z): 457.1 [M+H]⁺. ¹H NMR (400 MHz, CDCl₃) δ 7.86-7.76 (m, 2H), 7.58 (s, 1H), 7.36 (m, 1H), 7.18 (s, 1H), 6.99 (m, 2H), 5.56 (s, 2H), 4.96 (s, 2H), 2.64 (s, 6H).

The synthesis procedure of Examples (Compounds) 218-219 were similar to the procedure of Example 217.

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 217 Method 59 | +++ | 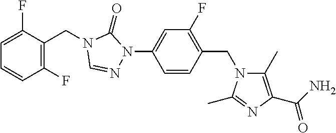 [SMILES: Cc1nc(C(N)=O)c(C)n1Cc1ccc(-n2ncn(Cc3c(F)cccc3F)c2=O)cc1F] | white solid, yield: 11.8% | ¹H NMR (400 MHz, CDCl₃) δ 7.86-7.76 (m, 2H), 7.58 (s, 1H), 7.36 (m, 1H), 7.18 (s, 1H), 6.99 (m, 2H), 5.56 (s, 2H), 4.96 (s, 2H), 2.64 (s, 6H). | 457.1 |
| | | 1-(4-(4-(2,6-difluorobenzyl)-5-oxo- | | | |
| | | 4,5-dihydro-1H-1,2,4-triazol- | | | |
| | | 1-yl)-2-fluorobenzyl)-2,5-dimethyl- | | | |
| | | 1H-imidazole-4-carboxamide | | | |
| Example 218 Method 59 | +++ | 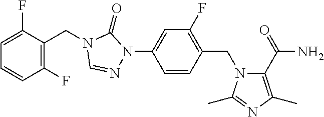 [SMILES: Cc1nc(C)n(Cc2ccc(-n3ncn(Cc4c(F)cccc4F)c3=O)cc2F)c1C(N)=O] | white solid, yield: 3% | ¹H NMR (400 MHz, CDCl₃) δ 7.91 (d, J = 11.9 Hz, 1H), 7.75 (d, J = 8.2 Hz, 1H), 7.59 (s, 1H), 7.37 (m, 1H), 6.98 (m, 2H), 6.70 (s, 1H), 5.10 (s, 2H), 4.97 | 457.1 |
| | | 1-(4-(4-(2,6-difluorobenzyl)-5-oxo- | | (s, 2H), 2.53 (s, 3H), | |
| | | 4,5-dihydro-1H-1,2,4-triazol- | | 2.42 (s, 3H). | |
| | | 1-yl)-2-fluorobenzyl)-2,4-dimethyl- | | | |
| | | 1H-imidazole-5-carboxamide | | | |
| Example 219 Method 59 | ++++ | 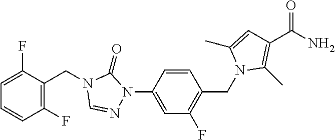 [SMILES: Cc1cc(C(N)=O)c(C)n1Cc1ccc(-n2ncn(Cc3c(F)cccc3F)c2=O)cc1F] | white solid, yield: 3.1% | ¹H NMR (400 MHz, CD₃OD) δ 8.00 (s, 1H), 7.78 (dd, J = 12.0, 1.9 Hz, 1H), 7.65 (d, J = 7.1 Hz, 1H), 7.49-7.38 (m, 1H), 7.05 (q, J = 8.0 Hz, 2H), 6.51 (t, J = 8.4 Hz, 1H), | 456.1 |
| | | 1-(4-(4-(2,6-difluorobenzyl)-5-oxo- | | 6.26 (s, 1H), 5.16 (s, | |
| | | 4,5-dihydro-1H-1,2,4-triazol- | | 2H), 5.02 (s, 2H), | |
| | | 1-yl)-2-fluorobenzyl)-2,5-dimethyl- | | 2.42 (s, 3H), 2.13 | |
| | | 1H-pyrrole-3-carboxamide | | (s, 3H). | |

### Example 220: Method 60

### 2-(3-fluoro-4-((4-(hydroxymethyl)-2-methyl-1H-imidazol-1-yl)methyl)phenyl)-5-phenyl-2,5,6,7-tetrahydro-3H-pyrrolo[2,1-c][1,2,4]triazol-3-one

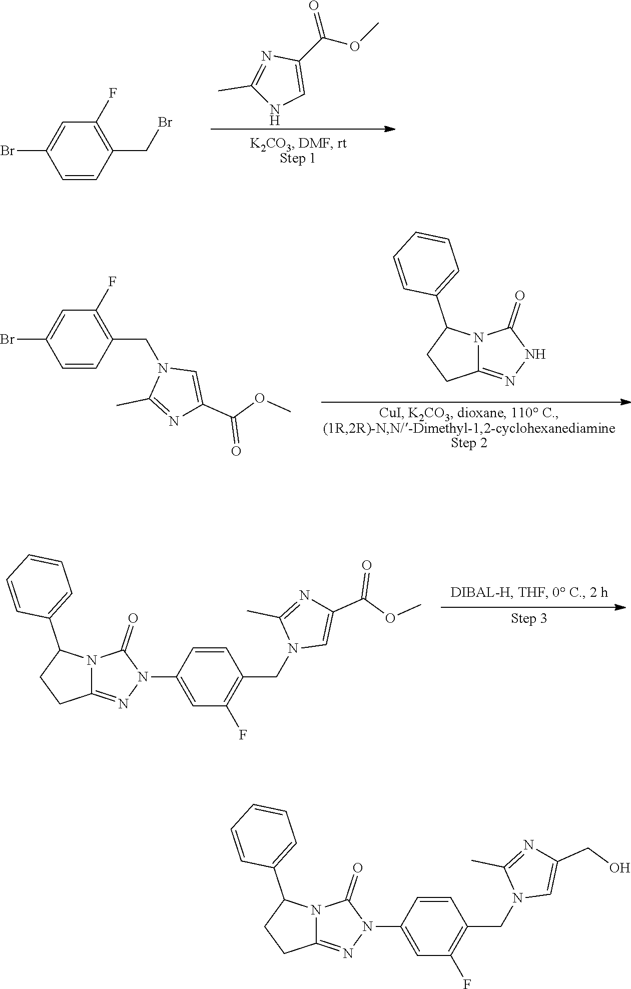

### Step 1: methyl 1-(4-bromo-2-fluorobenzyl)-2-methyl-1H-imidazole-4-carboxylate

To a stirred solution of 4-bromo-1-(bromomethyl)-2-fluorobenzene (100 mg, 0.37 mmol) and methyl 2-methyl-1H-imidazole-4-carboxylate (52.3 mg, 0.37 mmol) in DMF (5 mL) was added K₂CO₃ (154.7 mg, 1.12 mmol) at rt. The resulted mixture was stirred for additional 2 h at rt. The resulted mixture was diluted with water (20 mL). The resulted mixture was extracted with EtOAc (3×20 mL). The organic layers were combined, and the solvent was removed under vacuum and the crude was purified through silica gel chromatography (PE/EA=1:1) to give 1-(4-bromo-2-fluorobenzyl)-2,4-dimethyl-1H-imidazole as yellow solid (100 mg, 81.9% yield). LCMS (m/z): 327.3 [M+H]⁺

### Step 2: methyl 1-(2-fluoro-4-(3-oxo-5-phenyl-6,7-dihydro-3H-pyrrolo[2,1-c][1,2,4]triazol-2(5H)-yl)benzyl)-2-methyl-1H-imidazole-4-carboxylate

To a stirred solution of 5-phenyl-2H,5H,6H,7H-pyrrolo[2,1-c][1,2,4]triazol-3-one (93 mg, 0.46 mmol) and methyl 1-[(4-bromo-2-fluorophenyl)methyl]-2-methylimidazole-4-carboxylate (100 mg, 0.31 mmol) in dioxane (5 mL) were added copper(I) iodide (58 mg, 0.31 mmol) and (1R,2R)—N,N/′-Dimethyl-1,2-cyclohexanediamine (85 mg, 0.61 mmol) K₂CO₃ (154.7 mg, 1.12 mmol) at rt. The resulted mixture was stirred for additional 8 h at 110° C. under an atmosphere of Nitrogen. The mixture was allowed to cool down to rt. The resulted mixture was diluted with water (20 mL). The resulted mixture was extracted with EtOAc (3×20 mL). The combined organic layers were washed with brine (20 mL) and concentrated by reduced pressure, The residue was purified through silica gel chromatography (PE/EA=1:1) to give methyl 1-(2-fluoro-4-(3-oxo-5-phenyl-6,7-dihydro-3H-pyrrolo[2,1-c][1,2,4]triazol-2(5H)-yl)benzyl)-2-methyl-1H-imidazole-4-carboxylate as yellow solid (110 mg, 80% yield). LCMS (m/z): 448.2 [M+H]⁺

### Step 3: 2-(3-fluoro-4-((4-(hydroxymethyl)-2-methyl-1H-imidazol-1-yl)methyl)phenyl)-5-phenyl-2,5,6,7-tetrahydro-3H-pyrrolo[2,1-c][1,2,4]triazol-3-one

Diisobutylaluminium hydride in hexane (1 M,0.45 mL) was added to a solution of methyl 1-[(2-fluoro-4-{3-oxo-5-phenyl-5H,6H,7H-pyrrolo[2,1-c][1,2,4]triazol-2-yl}phenyl)methyl]-2-methylimidazole-4-carboxylate(100 mg, 0.22 mmol) in THF at 0° C. The mixture was stirred at 0° C. for 2 h, The resulted mixture was quenched with water (2 mL), and was filtered and the solvent was removed under vacuum. The residue was purified by reverse phase flash chromatography with the following conditions: column: Spherical C₁₈ 40-60 um, 40 g; Mobile phase B: ACN; Flow rate: 40 mL/min; Gradient: 30% B-60% B in 20 min; Detector: 254 nm. The fractions containing the desired product were collected at 55% B and concentrated under reduced pressure to afford the titled compound 2-(4-((2,4-dimethyl-1H-imidazol-1-yl)methyl)-3-fluorophenyl)-5-phenyl-2,5,6,7-tetrahydro-3H-pyrrolo[2,1-c][1,2,4]triazol-3-one (8 mg, 8.55% yield) as a white solid. LCMS (m/z): 420.1 [M+H]⁺. ¹H NMR (400 MHz, CDCl3) δ 7.85 (m, 2H), 7.42-7.34 (m, 3H), 7.24 (m, 2H), 7.13 (m, 1H), 6.82 (m, 1H), 5.30-5.26 (m, 1H), 5.07 (s, 2H), 4.60 (s, 2H), 3.01 (m, 4H), 2.58 (s, 3H).

The synthesis procedure of Examples (Compounds) 221-222 were similar to the procedure of Example 220.

| Compound | | | Appearance | | |
|---|---|---|---|---|---|
| No. | | | and | | MS(m/z) |
| Method | Activity | Structure and Name | Yield | ¹H NMR Data | [M + H]⁺ |
| Example 220 Method 60 | +++ | 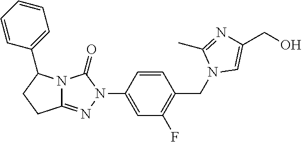 [SMILES: Cc1nc(CO)cn1Cc1ccc(-n2nc3n(c2=O)C(c2ccccc2)CC3)cc1F] | white solid, yield: 8.55% | ¹H NMR (400 MHz, CDCl₃) δ 7.85 (m, 2H), 7.42-7.34 (m, 3H), 7.24 (m, 2H), 7.13 (m, 1H), 6.82 (m, 1H), 5.30-5.26 (m, 1H), 5.07 (s, 2H), 4.60 (s, 2H), 3.01 (m, 4H), 2.58 (s, 3H). | 420.1 |
| | | 2-(3-fluoro-4-((4-(hydroxymethyl)- | | | |
| | | 2-methyl-1H-imidazol-1-yl) | | | |
| | | methyl)phenyl)-5-phenyl-2,5,6,7- | | | |
| | | tetrahydro-3H-pyrrolo[2,1-c] | | | |
| | | [1,2,4]triazol-3-one | | | |
| Example 221 Method 60 | ++ | 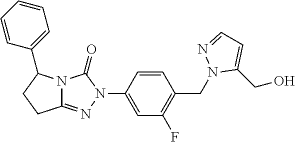 [SMILES: O=c1n(-c2ccc(Cn3nccc3CO)c(F)c2)nc2n1C(c1ccccc1)CC2] | white solid, yield: 22.1% | ¹H NMR (400 MHz, CDCl3) δ 7.76 (m, 1H), 7.68 (m, 1H), 7.41- 7.32 (m, 3H), 7.23 (m, 2H), 7.16 (s, 1H), 6.08 (s, 1H), 5.43 (s, 2H), 5.27 (s, 1H), 4.61 (s, 2H), 3.08-2.89 (m, 3H), 2.51 (m, 1H), 2.30 (s, 3H). | 420.2 |
| | | 2-(3-fluoro-4-((5-(hydroxymethyl)- | | | |
| | | 3-methyl-1H-pyrazol-1-yl)methyl) | | | |
| | | phenyl)-5-phenyl-2,5,6,7- | | | |
| | | tetrahydro-3H-pyrrolo[2,1-c] | | | |
| | | [1,2,4]triazol-3-one | | | |
| Example 222 Method 60 | ++++ | 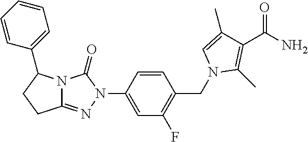 [SMILES: Cc1cn(Cc2ccc(-n3nc4n(c3=O)C(c3ccccc3)CC4)cc2F)c(C)c1C(N)=O] | white solid, yield: 5.8% | ¹H NMR (400 MHz, DMSO-d₆) δ 7.69-7.66 (m, 2H), 7.38-7.35 (m, 5H), 7.01 (t, J = 8.4 Hz, 1H), 6.69 (s, 2H), 6.48 (s, 1H), 5.30 (t, J = 6.0 Hz, 1H), 5.04 (s, 2H), 2.98-2.96 (m, 3H), 2.37-2.22 (m, 4H), 2.07 (s, 3H). | 446.2 |
| | | 1-(2-fluoro-4-(3-oxo-5-phenyl- | | | |
| | | 6,7-dihydro-3H-pyrrolo[2,1-c] | | | |
| | | [1,2,4]triazol-2(5H)-yl)benzyl)- | | | |
| | | 2,4-dimethyl-1H-pyrrole-3- | | | |
| | | carboxamide | | | |

### Example 223: Method 61

### 2-(4-((2-amino-5-fluoropyridin-4-yl)oxy)-3-fluorophenyl)-4-(2,6-difluorobenzyl)-2,4-dihyd ro-3H-1,2,4-triazol-3-one

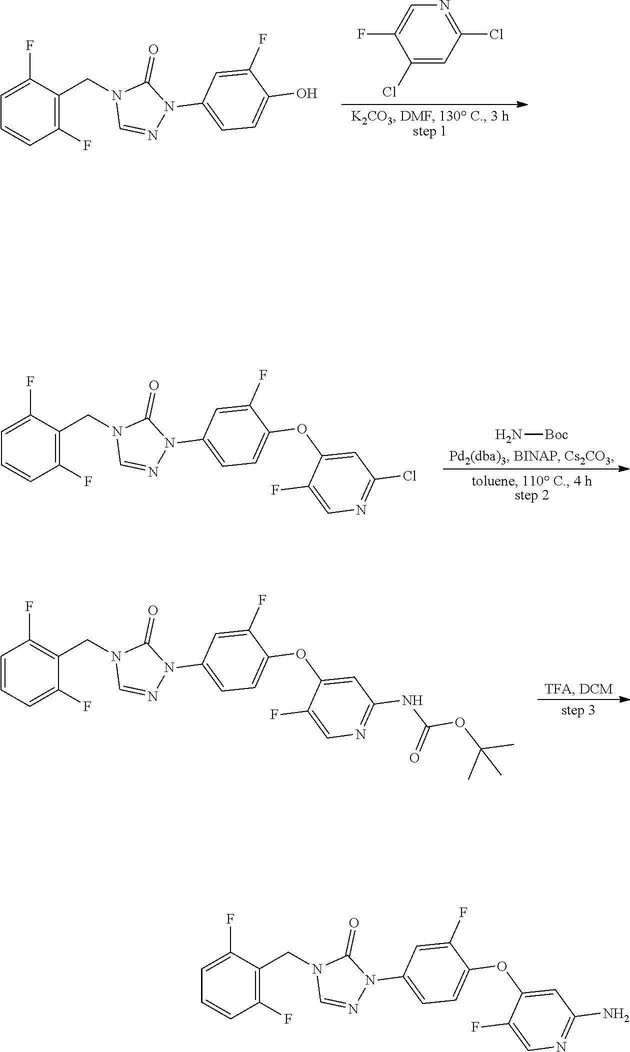

### Step 1: 2-(4-((2-chloro-5-fluoropyridin-4-yl)oxy)-3-fluorophenyl)-4-(2,6-difluorobenzyl)-2,4-dihydro-3H-1,2,4-triazol-3-one

4-(2,6-difluorobenzyl)-2-(3-fluoro-4-hydroxyphenyl)-2,4-dihydro-3H-1,2,4-triazol-3-one (1 g, 3.115 mmoL),2,4-dichloro-5-fluoropyridine (568 mg, 3.427 mmoL) and K₂CO₃ (860 mg, 6.23 mmoL) were dissolved in 10 mL DMF. The resulted mixture was stirred at 130° C. for 3 h. Water was added to the solution and the reaction mixture was extracted with EtOAc (50 mL×3). The organic layers were combined and washed with water. It was evaporated to dryness and purified by column chromatography (PE/EA=3/1) to give 2-(4-((2-chloro-5-fluoropyridin-4-yl)oxy)-3-fluorophenyl)-4-(2,6-difluorobenzyl)-2,4-dihydro-3H-1,2,4-triazol-3-one as a white solid (702 mg, yield: 50%). MS (m/z): 451.3 [M+H]⁺.

### Step 2: tert-butyl (4-(4-(4-(2,6-difluorobenzyl)-5-oxo-4,5-dihydro-1H-1,2,4-triazol-1-yl)-2-fluorophenoxy)-5-fluoropyridin-2-yl)carbamate

To a solution of Tris(dibenzylideneacetonyl)bis-palladium (0.150 eq, 152 mg, 0.166 mmol), tert-Butyl carbamate (1.50 eq, 195 mg, 1.66 mmol), Cs₂CO₃ (2.00 eq, 723 mg, 2.22 mmol) and BINAP (0.300 eq, 207 mg, 0.333 mmol) in toluene (5 mL), was added 2-[4-[(2-chloro-5-fluoro-4-pyridyl)oxy]-3-fluoro-phenyl]-4-[(2,6-difluorophenyl)methyl]-1,2,4-triazol-3-one (1.00 eq, 500 mg, 1.11 mmol) under N₂ at room temperature. The mixture was heated to 110° C. and stirred for 4 h. Then to the reaction mixture was added water (20 mL) and extracted with EtOAc (20 mL×3). The organics were then combined and dried (Na₂SO₄) before concentration to dryness. The crude was purified with flash chromatography (Silica gel column, 30 g, EtOAc/PE, 0 to 50%) to give the product tert-butyl N-[4-[4-[4-[(2,6-difluorophenyl)methyl]-5-oxo-1,2,4-triazol-1-yl]-2-fluoro-phenoxy]-5-fluoro-2-pyridyl]carbamate (300 mg, 0.452 mmol, 40.71% yield) as a yellow gum. MS (m/z): 532.2 [M+H]⁺.

### Step 3: 2-(4-((2-amino-5-fluoropyridin-4-yl)oxy)-3-fluorophenyl)-4-(2,6-difluorobenzyl)-2,4-dihydro-3H-1,2,4-triazol-3-one

2-(4-((2-amino-5-fluoropyridin-4-yl)oxy)-3-fluorophenyl)-4-(2,6-difluorobenzyl)-2,4-dihydro-3H-1,2,4-triazol-3-one (33 mg, 0.062 mmoL) was dissolved in 2 mL DCM. 1 mL DCM/TFA (v/v=1/1) was added to that solution at 0° C. The reaction mixture was stirred at r.t. for 30 min. The solvent was evaporated to dryness and purified by prep-HPLC to give 2-(4-((2-amino-5-fluoropyridin-4-yl)oxy)-3-fluorophenyl)-4-(2,6-difluorobenzyl)-2,4-dihydro-3H-1,2,4-triazol-3-one as a yellow solid (21.4 mg, yield: 79.13%). MS (m/z): 432.2 [M+H]⁺. ¹H NMR (400 MHz, DMSO-d₆) δ 8.40 (s, 1H), 7.98-7.88 (m, 2H), 7.81-7.78 (m, 1H), 7.55-5.45 (m, 2H), 7.17 (t, J=8.1 Hz, 2H), 5.86 (s, 2H), 5.82-5.77 (m, 1H), 5.00 (s, 2H).

The synthesis procedure of Examples (Compounds) 224-401 were similar to the procedure of Example 223.

| | | | Ap- | | |
|---|---|---|---|---|---|
| Com- | | | pear- | | MS |
| pound | Ac- | | ance | | (m/z) |
| No. | tiv- | | and | | [M + |
| Method | ity | Structure and Name | Yield | ¹H NMR Data | H]⁺ |
| Example 223 Method 61 | +++ | 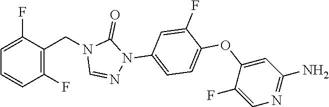 [SMILES: Nc1cc(Oc2ccc(-n3ncn(Cc4c(F)cccc4F)c3=O)cc2F)c(F)cn1] | yellow solid, yield: 79.13% | ¹H NMR (400 MHz, DMSO-d₆) δ 8.40 (s, 1H), 7.98-7.88 (m, 2H), 7.81-7.78 (m, 1H), 7.55-5.45 (m, 2H), 7.17 (t, J = 8.1 Hz, 2H), 5.86 (s, 2H), 5.82-5.77 (m, 1H), 5.00 (s, 2H). | 432.2 |
| | | 2-(4-((2-amino-5-fluoropyridin-4-yl) | | | |
| | | oxy)-3-fluorophenyl)-4-(2,6-di- | | | |
| | | fluorobenzyl)-2,4-dihydro-3H-1,2,4- | | | |
| | | triazol-3-one | | | |
| Example | ++++ | 1-(4-(2-chloro-4-(4-(2,6-difluoro- | white | ¹H NMR (400 MHz, CDCl₃) δ | 527.0 |
| 224 | | benzyl)-5-oxo-4,5-dihydro-1H-1,2,4- | solid, | 8.18 (d, J = 2.6 Hz, 1H), 7.96 (dd, | |
| Method | | triazol-1-yl)phenoxy)-3-fluoropyridin- | yield: | J = 8.9, 2.6 Hz, 1H), 7.75 (d, J = 5.8 | |
| 61 | | 2-yl)-3-methylazetidine-3-carbonitrile | 12.1% | Hz, 1H), 7.61 (s, 1H), 7.37 (tt, J = | |
| | | | | 8.4, 6.5 Hz, 1H), 7.16 (d, J = 9.0 | |
| | | | | Hz, 1H), 6.98 (dd, J = 8.2, 7.7 Hz, | |
| | | | | 2H), 6.07 (t, J = 5.7 Hz, 1H), 4.97 | |
| | | | | (s, 2H), 4.56 (d, J = 7.3 Hz, 2H), | |
| | | | | 4.11 (d, J = 7.3 Hz, 2H), 1.78 (s, | |
| | | | | 3H). | |
| Example | +++ | 1-[4-(4-{4-[(2,6-difluorophenyl) | white | ¹H NMR (400 MHz, DMSO-d₆) δ | 508.1 |
| 225 | | methyl]-5-oxo-1,2,4-triazol-1-yl}-2- | solid, | 8.02 (s, 1H), 7.91 (dd, J = 11.8, 2.4 | |
| Method | | fluorophenoxy)-5-methylpyrimidin-2- | yield: | Hz, 1H), 7.82 (dd, J = 8.9, 1.3 Hz, | |
| 61 | | yl]-3-methylazetidine-3-carbonitrile | | 1H), 7.61 (s, 1H), 7.40-7.34 (m, | |
| | | | | 1H), 7.19 (t, J = 8.6 Hz, 1H), 7.02- | |
| | | | | 6.96 (m, 2H), 4.98 (s, 2H), 4.25 (d, | |
| | | | | J = 7.7 Hz, 2H), 3.80 (d, J = 6.3 | |
| | | | | Hz, 2H), 2.19 (s, 3H), 1.64 (s, 3H). | |
| Example | ++++ | 1-(4-(4-(4-(2,6-difluorobenzyl)-5- | white | ¹H NMR (400 MHz, DMSO-d₆) δ | 511.2 |
| 226 | | oxo-4,5-dihydro-1H-1,2,4-triazol-1- | solid, | 8.39 (s, 1H), 7.92 (dd, J = 12.4, 2.5 | |
| Method | | yl)-2-fluorophenoxy)-3-fluoropyridin- | yield: | Hz, 1H), 7.84-7.71 (m, 2H), 7.55- | |
| 61 | | 2-yl)-3-methylazetidine-3-carbonitrile | | 7.42 (m, 2H), 7.22-7.10 (m, | |
| | | | | 2H), 6.32 (t, J = 5.5 Hz, 1H), 4.98 | |
| | | | | (s, 2H), 4.38 (dd, J = 8.3, 1.5 Hz, | |
| | | | | 2H), 4.07 (dd, J = 8.3, 1.5 Hz, 2H), | |
| | | | | 1.67 (s, 3H). | |
| Example 227 Method 61 | ++++ | 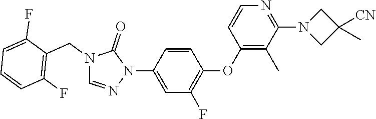 [SMILES: Cc1c(Oc2ccc(-n3ncn(Cc4c(F)cccc4F)c3=O)cc2F)ccnc1N1CC(C)(C#N)C1] | white solid, yield: | ¹H NMR (400 MHz, DMSO-d₆) δ 8.38 (s, 1H), 7.95-7.84 (m, 2H), 7.80-7.69 (m, 1H), 7.55-7.42 (m, 1H), 7.34 (t, J = 9.0 Hz, 1H), 7.17 (t, J = 8.1 Hz, 2H), 6.15 (d, J = 5.7 Hz, 1H), 4.99 (s, 2H), 4.37 (d, J = 8.2 Hz, 2H), 4.05 (d, J = 8.3 Hz, 2H), 2.10 (s, 3H), 1.65 (s, 3H). | 507.2 |
| | | 1-(4-(4-(4-(2,6-difluorobenzyl)-5- | | | |
| | | oxo-4,5-dihydro-1H-1,2,4-triazol-1- | | | |
| | | yl)-2-fluorophenoxy)-3-methylpyridin- | | | |
| | | 2-yl)-3-methylazetidine-3-carbonitrile | | | |
| Example 228 Method 61 | ++++ | 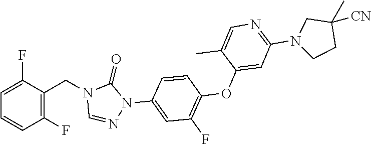 [SMILES: Cc1cnc(N2CCC(C)(C#N)C2)cc1Oc1ccc(-n2ncn(Cc3c(F)cccc3F)c2=O)cc1F] | white solid, yield: | ¹H NMR (400 MHz, DMSO-d₆) δ 8.39 (s, 1H), 7.97-7.86 (m, 2H), 7.78 (d, J = 9.0 Hz, 1H), 7.54- 7.44 (m, 1H), 7.39 (t, J = 9.0 Hz, 1H), 7.17 (t, J = 8.1 Hz, 2H), 5.60 (s, 1H), 5.00 (s, 2H), 3.76 (d, J = 10.6 Hz, 1H), 3.35 (s, 2H), 3.20 (d, J = 10.7 Hz, 1H), 2.37-2.24 (m, 1H), 2.13 (s, 3H), 2.05-1.95 (m, 1H), 1.43 (s, 3H). | 521.2 |
| | | 1-(4-(4-(4-(2,6-difluorobenzyl)-5- | | | |
| | | oxo-4,5-dihydro-1H-1,2,4-triazol-1- | | | |
| | | yl)-2-fluorophenoxy)-5-methylpyridin- | | | |
| | | 2-yl)-3-methylpyrrolidine-3-carbonitrile | | | |
| Example 229 Method 61 | ++++ | 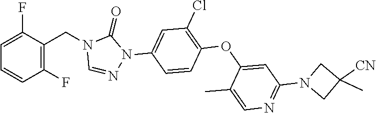 [SMILES: Cc1cnc(N2CC(C)(C#N)C2)cc1Oc1ccc(-n2ncn(Cc3c(F)cccc3F)c2=O)cc1Cl] | yellow solid, yield: 15% | 1H NMR (400 MHz, CDCl₃) δ 8.20 (d, J = 2.5 Hz, 1H), 7.98 (dd, J = 8.9, 2.5 Hz, 1H), 7.94 (s, 1H), 7.63 (s, 1H), 7.39 (m, 1H), 7.15 (d, J = 8.9 Hz, 1H), 7.00 (m, 2H), 5.34 (s, 1H), 5.00 (s, 2H), 4.26 (d, J = 6.7 Hz, 2H), 3.85 (s, 2H), 2.23 (s, 3H), 1.70 (s, 3H) | 523.1 |
| | | 1-(4-(2-chloro-4-(4-(2,6-difluoro- | | | |
| | | benzyl)-5-oxo-4,5-dihydro-1H-1,2,4- | | | |
| | | triazol-1-yl)phenoxy)-5-methylpyridin- | | | |
| | | 2-yl)-3-methylazetidine-3-carbonitrile | | | |
| Example 230 Method 61 | ++++ | 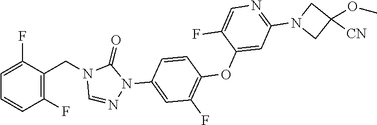 [SMILES: COC1(C#N)CN(c2cc(Oc3ccc(-n4ncn(Cc5c(F)cccc5F)c4=O)cc3F)c(F)cn2)C1] | yellow solid, yield: 20% | NMR (400 MHz,CDCl₃) δ 8.11 (d, J = 3.8 Hz, 1H), 8.04 (dd, J = 11.8, 2.5 Hz, 1H), 7.92 (ddd, J = 9.0, 2.3, 1.5 Hz, 1H), 7.64 (s, 1H), 7.38 (m, 1H), 7.26 (m, 1H), 6.99 (m, 2H), 5.53 (m, 1H), 4.99 (s, 2H), 4.55 (d, J = 9.8 Hz, 2H), 4.29 (d, J = 9.4 Hz, 2H), 3.45 (m, 3H). | 527.0 |
| | | 1-(4-(4-(4-(2,6-difluorobenzyl)-5- | | | |
| | | oxo-4,5-dihydro-1H-1,2,4-triazol-1- | | | |
| | | yl)-2-fluorophenoxy)-5-fluoropyridin- | | | |
| | | 2-yl)-3-methoxyazetidine-3-carbonitrile | | | |
| Example 231 Method 61 | ++++ | 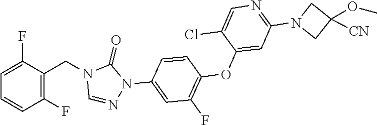 [SMILES: COC1(C#N)CN(c2cc(Oc3ccc(-n4ncn(Cc5c(F)cccc5F)c4=O)cc3F)c(Cl)cn2)C1] | yellow solid, yield: 19% | NMR (400 MHz, CDCl₃) δ 8.11 (d, J = 3.8 Hz, 1H), 8.04 (dd, J = 11.8, 2.5 Hz, 1H), 7.92 (ddd, J = 9.0, 2.3, 1.5 Hz, 1H), 7.64 (s, 1H), 7.38 (m, 1H), 7.26 (m, 1H), 6.99 (m, 2H), 5.53 (m, 1H), 4.99 (s, 2H), 4.55 (d, J = 9.8 Hz, 2H), 4.29 (d, J = 9.4 Hz, 2H), 3.45 (m, 3H). | 542.9 |
| | | 1-(5-chloro-4-(4-(4-(2,6-difluoro- | | | |
| | | benzyl)-5-oxo-4,5-dihydro-1H-1,2,4- | | | |
| | | triazol-1-yl)-2-fluorophenoxy)pyridin- | | | |
| | | 2-yl)-3-methoxyazetidine-3-carbonitrile | | | |
| Example 232 Method 61 | ++++ | 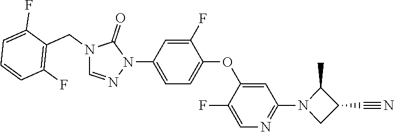 [SMILES: C[C@H]1[C@H](C#N)CN1c1cc(Oc2ccc(-n3ncn(Cc4c(F)cccc4F)c3=O)cc2F)c(F)cn1] | yellow solid, yield: 6% | 1H NMR (400 MHz, CDCl₃) δ 8.14 (d, J = 2.9 Hz, 1H), 8.02 (dd, J = 11.9, 2.3 Hz, 1H), 7.91 (d, J = 8.6 Hz, 1H), 7.65 (s, 1H), 7.39 (m, 1H), 7.24 (d, J = 8.7 Hz, 1H), 7.01 (dd, J = 13.4, 5.7 Hz, 2H), 5.56 (d, J = 5.2 Hz, 1H), 5.00 (s, 2H), 4.60 (s, 1H), 4.17 (d, J = 19.5 Hz, 2H), 3.66 (s, 1H), 1.58 (d, J = 5.1 Hz, | 511.1 |
| | | (2S,3R)-1-(4-(4-(4-(2,6-difluoro- | | 3H). | |
| | | benzyl)-5-oxo-4,5-dihydro-1H-1,2,4- | | | |
| | | triazol-1-yl)-2-fluorophenoxy)-5- | | | |
| | | fluoropyridin-2-yl)-2-methylazetidine- | | | |
| | | 3-carbonitrile | | | |
| Example 233 Method 61 | ++++ | 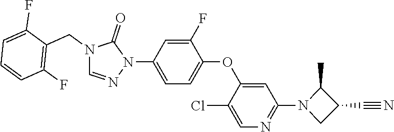 [SMILES: C[C@H]1[C@H](C#N)CN1c1cc(Oc2ccc(-n3ncn(Cc4c(F)cccc4F)c3=O)cc2F)c(Cl)cn1] | yellow solid, yield: 15% | 1H NMR (400 MHz, CDCl₃) δ 8.22 (s, 1H), 8.04 (dd, J = 11.8, 2.2 Hz, 1H), 7.93 (d, J = 8.2 Hz, 1H), 7.65 (s, 1H), 7.39 (ddd, J = 13.2, 7.5, 4.2 Hz, 1H), 7.26 (m, 2H), 7.01 (dd, J = 13.4, 5.8 Hz, 2H), 5.48 (s, 1H), 5.00 (s, 2H), 4.69 (s, 1H), 4.25 (s, 2H), 3.71 (s, 1H), 1.58 (d, J = 3.9 Hz, 3H). | 527.0 |
| | | (2S,3R)-1-(5-chloro-4-(4-(4-(2,6-di- | | | |
| | | fluorobenzyl)-5-oxo-4,5-dihydro-1H- | | | |
| | | 1,2,4-triazol-1-yl)-2-fluorophenoxy) | | | |
| | | pyridin-2-yl)-2-methylazetidine- | | | |
| | | 3-carbonitrile | | | |
| Example 234 Method 61 | +++ | 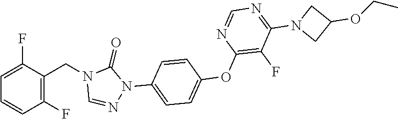 [SMILES: CCOC1CN(c2ncnc(Oc3ccc(-n4ncn(Cc5c(F)cccc5F)c4=O)cc3)c2F)C1] | white solid, yield: 45.6% | ¹H NMR (400 MHz, CDCl₃) δ 8.12-7.91 (m, 3H), 7.57 (s, 1H), 7.36 (tt, J = 8.4, 6.5 Hz, 1H), 7.23- 7.13 (m, 2H), 7.04-6.89 (m, 2H), 4.98 (s, 2H), 4.46 (t, J = 5.8 Hz, 3H), 4.25-4.12 (m, 2H), 3.50 (q, J = 7.0 Hz, 2H), 1.25 (t, J = 7.0 Hz, 3H). | 499.0 |
| | | 4-(2,6-difluorobenzyl)-2-(4-((6-(3- | | | |
| | | ethoxyazetidin-1-yl)-5-fluoro- | | | |
| | | pyrimidin-4-yl)oxy)phenyl)-2,4-dihydro- | | | |
| | | 3H-1,2,4-triazol-3-one | | | |
| Example 235 Method 61 | ++++ | 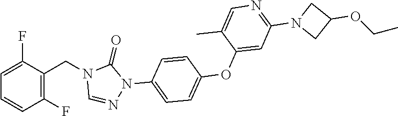 [SMILES: CCOC1CN(c2cc(Oc3ccc(-n4ncn(Cc5c(F)cccc5F)c4=O)cc3)c(C)cn2)C1] | white solid, yield: 47.6% | ¹H NMR (400 MHz, CDCl₃) δ 8.12 (d, J = 8.8 Hz, 2H), 7.86 (s, 1H), 7.64 (s, 1H), 7.43-7.36 (m, 1H), 7.15 (d, J = 8.8 Hz, 1H), 7.07-6.99 (m, 2H), 5.38 (s, 1H), 5.01 (s, 2H), 4.35 (s, 2H), 4.12 (q, J = 7.2 Hz, 1H), 3.99 (s, 3H), 3.43 (q, J = 6.8 Hz, 2H), 2.23 (s, 3H), 1.18 (t, J = | 494.1 |
| | | 4-(2,6-difluorobenzyl)-2-(4-((2-(3- | | 6.8 Hz, 3H). | |
| | | ethoxyazetidin-1-yl)-5-methylpyridin- | | | |
| | | 4-yl)oxy)phenyl)-2,4-dihydro- | | | |
| | | 3H-1,2,4-triazol-3-one | | | |
| Example 236 Method 61 | ++++ | 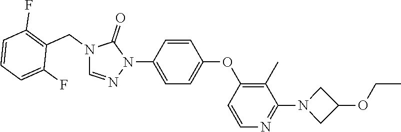 [SMILES: CCOC1CN(c2nccc(Oc3ccc(-n4ncn(Cc5c(F)cccc5F)c4=O)cc3)c2C)C1] | white solid, yield: 16.68% | 1H NMR (400 MHz, CDCl3) δ 7.93 (ddd, J = 12.1, 7.1, 4.5 Hz, 3H), 7.58 (s, 1H), 7.37 (tt, J = 8.4, 6.5 Hz, 1H), 7.11-6.90 (m, 4H), 6.16 (d, J = 5.8 Hz, 1H), 4.98 (s, 2H), 4.45-4.28 (m, 3H), 4.04 (dd, J = 8.2, 3.4 Hz, 2H), 3.50 (q, J = 7.0 Hz, 2H), 2.14 (d, J = 8.0 Hz, 3H), 1.24 (t, J = 7.0 Hz, 3H). | 494.0 |
| | | 4-(2,6-difluorobenzyl)-2-(4-((2-(3- | | | |
| | | ethoxyazetidin-1-yl)-3-methylpyridin- | | | |
| | | 4-yl)oxy)phenyl)-2,4-dihydro- | | | |
| | | 3H-1,2,4-triazol-3-one | | | |
| Example 237 Method 61 | ++++ | 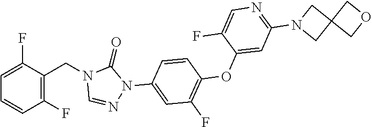 [SMILES: O=c1n(Cc2c(F)cccc2F)cnn1-c1ccc(Oc2cc(N3CC4(COC4)C3)ncc2F)c(F)c1] | white solid, yield: 11.7% | ¹H NMR (400 MHz, CDCl₃) δ 8.04-7.94 (m, 2H), 7.89-7.82 (m, 1H), 7.63 (s, 1H), 7.39 (dq, J = 8.4, 6.6 Hz, 1H), 7.21 (t, J = 8.7 Hz, 1H), 7.04-6.96 (m, 2H), 5.53 (d, J = 5.4 Hz, 1H), 4.98 (d, J = 10.0 Hz, 2H), 4.79 (s, 4H), 4.03 (s, 4H). | 514.2 |
| | | 4-(2,6-difluorobenzyl)-2-(3-fluoro- | | | |
| | | 4-((5-fluoro-2-(2-oxa-6-azaspiro | | | |
| | | [3.3]heptan-6-yl)pyridin-4-yl)oxy) | | | |
| | | phenyl)-2,4-dihydro-3H-1,2,4-triazol- | | | |
| | | 3-one | | | |
| Example 238 Method 61 | ++++ | 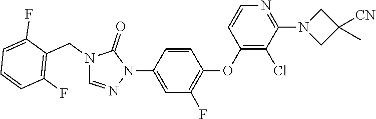 [SMILES: CC1(C#N)CN(c2nccc(Oc3ccc(-n4ncn(Cc5c(F)cccc5F)c4=O)cc3F)c2Cl)C1] | white solid, yield: 17% | ¹H NMR (400 MHz, DMSO-d₆) δ 8.40 (s, 1H), 7.98-7.88 (m, 2H), 7.82-7.74 (m, 1H), 7.54-7.41 (m, 2H), 7.17 (t, J = 8.1 Hz, 2H), 6.25 (d, J = 5.6 Hz, 1H), 4.99 (s, 2H), 4.48 (d, J = 8.6 Hz, 2H), 4.17 (d, J = 8.6 Hz, 2H), 1.65 (s, 3H). | 527.1 |
| | | 1-(3-chloro-4-(4-(4-(2,6-difluoro- | | | |
| | | benzyl)-5-oxo-4,5-dihydro-1H-1,2,4- | | | |
| | | triazol-1-yl)-2-fluorophenoxy)pyridin- | | | |
| | | 2-yl)-3-methylazetidine-3-carbonitrile | | | |
| Example 239 Method 61 | ++++ | 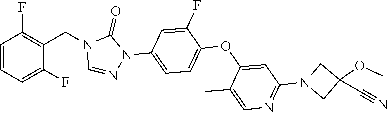 [SMILES: COC1(C#N)CN(c2cc(Oc3ccc(-n4ncn(Cc5c(F)cccc5F)c4=O)cc3F)c(C)cn2)C1] | yellow solid, yield: 6.86% | ¹H NMR (400 MHz, CDCl₃) δ 8.02-7.85 (m, 3H), 7.64 (s, 1H), 7.45-7.34 (m, 1H), 7.20 (t, J = 8.7 Hz, 1H), 7.05-6.95 (m, 2H), 5.44 (s, 1H), 5.00 (s, 2H), 4.41 (s, 2H), 4.15 (s, 2H), 3.44 (s, 3H), 2.25 (s, 3H). | 522.9 |
| | | 1-(4-(4-(4-(2,6-difluorobenzyl)-5- | | | |
| | | oxo-4,5-dihydro-1H-1,2,4-triazol-1- | | | |
| | | yl)-2-fluorophenoxy)-5-methylpyridin- | | | |
| | | 2-yl)-3-methoxyazetidine-3-carbonitrile | | | |
| Example 240 Method 61 | ++++ | 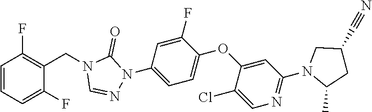 [SMILES: C[C@H]1C[C@@H](C#N)CN1c1cc(Oc2ccc(-n3ncn(Cc4c(F)cccc4F)c3=O)cc2F)c(Cl)cn1] | yellow solid, yield: 7.76% | ¹H NMR (400 MHz, CDCl₃) δ 8.17 (s, 1H), 8.06-8.00 (m, 1H), 7.91 (d, J = 9.0 Hz, 1H), 7.64 (s, 1H), 7.38 (d, J = 8.4 Hz, 1H), 7.24 (d, J = 8.7 Hz, 1H), 7.00 (t, J = 7.9 Hz, 2H), 5.60 (d, J = 10.5 Hz, 1H), 5.00 (s, 2H), 3.76 (s, 2H), 3.17 (s, 1H), 2.58-2.46 (m, 1H), 2.23-1.97 | 541.0 |
| | | (3R,5S)-1-(5-chloro-4-(4-(4-(2,6-di- | | (m, 2H), 1.25 (s, 3H). | |
| | | fluorobenzyl)-5-oxo-4,5-dihydro-1H- | | | |
| | | 1,2,4-triazol-1-yl)-2-fluorophenoxy) | | | |
| | | pyridin-2-yl)-5-methylpyrrolidine- | | | |
| | | 3-carbonitrile | | | |
| Example 241 Method 61 | ++++ | 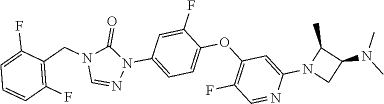 [SMILES: C[C@H]1[C@@H](N(C)C)CN1c1cc(Oc2ccc(-n3ncn(Cc4c(F)cccc4F)c3=O)cc2F)c(F)cn1] | yellow solid, yield: 17% | 1H NMR (400 MHz, CDCl₃) δ 8.09 (s, 1H), 7.99 (dd, 1H), 7.86 (dt, J = 9.0 Hz, 1H), 7.62 (s, 1H), 7.38 (m, 1H), 7.22 (t, 1H), 7.04-6.95 (t, 2H), 5.52 (s, 1H), 4.99 (s, 2H), 3.99 (s, 1H), 2.41 (s, 6H), 1.59 (m, 3H), 1.43 (s, 3H). | 529.2 |
| | | 4-(2,6-difluorobenzyl)-2-(4-((2- | | | |
| | | ((2S,3S)-3-(dimethylamino)-2-methyl- | | | |
| | | azetidin-1-yl)-5-fluoropyridin-4-yl) | | | |
| | | oxy)-3-fluorophenyl)-2,4-dihydro- | | | |
| | | 3H-1,2,4-triazol-3-one | | | |
| Example 242 Method 61 | ++++ | 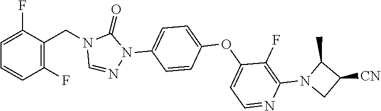 [SMILES: C[C@H]1[C@@H](C#N)CN1c1nccc(Oc2ccc(-n3ncn(Cc4c(F)cccc4F)c3=O)cc2)c1F] | white solid, yield: 12% | 1H NMR (400 MHz, DMSO-d₆) δ 8.33 (s, 1H), 8.01-7.78 (m, 3H), 7.49 (t, J = 7.5 Hz, 1H), 7.26 (d, J = 9.0 Hz, 2H), 7.17 (t, J = 8.1 Hz, 2H), 6.39 (t, J = 5.5 Hz, 1H), 4.99 (s, 2H), 4.70-4.56 (m, 1H), 4.31 (d, J = 8.2 Hz, 1H), 4.12 (t, J = 6.9 Hz, 1H), 3.57 (d, J = 7.5 Hz, 1H), | 493.2 |
| | | (2S,3S)-1-(4-(4-(4-(2,6-difluoro- | | 1.50 (s, J = 6.1 Hz, 3H). | |
| | | benzyl)-5-oxo-4,5-dihydro-1H-1,2,4- | | | |
| | | triazol-1-yl)phenoxy)-3-fluoro- | | | |
| | | pyridin-2-yl)-2-methylazetidine-3- | | | |
| | | carbonitrile | | | |
| Example 243 Method 61 | ++++ | 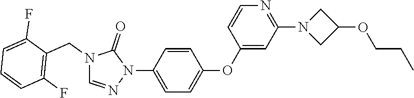 [SMILES: CCCOC1CN(c2cc(Oc3ccc(-n4ncn(Cc5c(F)cccc5F)c4=O)cc3)ccn2)C1] | white solid, yield: 21% | ¹H NMR (400 MHz, MeOH-d₄) δ 8.06 (d, J = 9.2 Hz, 3H), 7.84 (d, J = 7.3 Hz, 1H), 7.50-7.39 (m, 1H), 7.29 (d, J = 9.0 Hz, 2H), 7.05-7.09 (m, 2H), 6.60 (dd, J = 7.2, 2.3 Hz, 1H), 5.97 (d, J = 2.2 Hz, 1H), 5.06 (s, 2H), 4.46-4.51 (m, 1H), 4.37 (t, | 494.0 |
| | | 4-(2,6-difluorobenzyl)-2-(4-((2-(3- | | 2H), 4.09-3.96 (m, 2H), 3.41 (t, | |
| | | propoxyazetidin-1-yl)pyridin-4-yl) | | 2H), 1.60 (q, 2H), 0.94 (t, 3H). | |
| | | oxy)phenyl)-2,4-dihydro-3H-1,2,4- | | | |
| | | triazol-3-one | | | |
| Example 244 Method 61 | +++ | 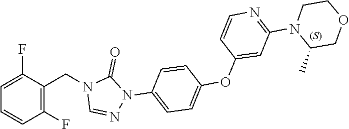 | white solid, yield: 8% | ¹H NMR (400 MHz, MeOH-d₄) δ 8.06-8.08 (m, 3H), 7.93 (d, J = 7.2 Hz, 1H), 7.42-7.52 (m, 1H), 7.31 (d, J = 9.1 Hz, 2H), 7.05-7.09 (m, 2H), 6.66 (d, J = 7.2 Hz, 1H), 6.57 (d, J = 2.3 Hz, 1H), 5.06 (s, 2H), 4.07-3.97 (m, 2H), 3.72-3.76 (m, 2H), 3.56-3.62 (m, 2H), 3.49- 3.39 (m, 1H), 1.33 (d, J = 6.8 Hz, | 480.2 |
| | | (S)-4-(2,6-difluorobenzyl)-2-(4-((2- | | 3H). | |
| | | (3-methylmorpholino)pyridin-4-yl) | | | |
| | | oxy)phenyl)-2,4-dihydro-3H-1,2,4- | | | |
| | | triazol-3-one | | | |
| Example 245 Method 61 | +++ | 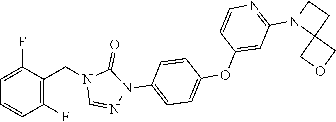 [SMILES: O=c1n(Cc2c(F)cccc2F)cnn1-c1ccc(Oc2ccnc(N3CCC34COC4)c2)cc1] | white solid, yield: 6% | ¹H NMR (400 MHz, MeOH-d₄) δ 8.14-8.00 (m, 3H), 7.94 (d, J = 7.9 Hz, 1H), 7.42-7.79 (m, 1H), 7.32 (d, J = 9.1 Hz, 2H), 7.07 (t, 2H), 6.68-6.70 (m, 2H), 5.05 (s, 2H), 4.79-4.84 (m, 4H), 4.04 (t, 2H), 2.63 (t, 2H). | 478.1 |
| | | 2-(4-((2-(6-oxa-1-azaspiro[3.3]heptan- | | | |
| | | 1-yl)pyridin-4-yl)oxy)phenyl)-4- | | | |
| | | (2,6-difluorobenzyl)-2,4-dihydro- | | | |
| | | 3H-1,2,4-triazol-3-one | | | |
| Example 246 Method 61 | ++++ | 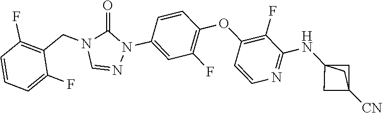 [SMILES: N#CC12CC(Nc3nccc(Oc4ccc(-n5ncn(Cc6c(F)cccc6F)c5=O)cc4F)c3F)(C1)C2] | yellow solid, yield: 6% | ¹H NMR (400 MHz, CD₃OD) δ 8.53 (s, 1H), 8.04 (s, 1H), 7.90 (dd, J = 12.2, 2.5 Hz, 1H), 7.83-7.76 (m, 1H), 7.71 (dd, J = 5.8, 0.7 Hz, 1H), 7.50-7.41 (m, 1H), 7.28 (t, J = 8.9 Hz, 1H), 7.07 (t, J = 8.1 Hz, 2H), 6.13 (t, J = 5.4 Hz, 1H), 5.05 (s, 2H), 2.62 (s, 6H). | 523.1 |
| | | 3-((4-(4-(4-(2,6-difluorobenzyl)-5- | | | |
| | | oxo-4,5-dihydro-1H-1,2,4-triazol-1- | | | |
| | | yl)-2-fluorophenoxy)-3-fluoropyridin- | | | |
| | | 2-yl)amino)bicyclo[1.1.1] | | | |
| | | pentane-1-carbonitrile | | | |
| Example 247 Method 61 | ++++ | 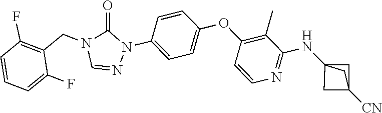 [SMILES: Cc1c(Oc2ccc(-n3ncn(Cc4c(F)cccc4F)c3=O)cc2)ccnc1NC12CC(C#N)(C1)C2] | yellow solid, yield: 2% | ¹H NMR (400 MHz, CD₃OD) δ 8.06-7.97 (m, 3H), 7.84 (d, J = 7.1 Hz, 1H), 7.49-7.43 (m, 1H), 7.25-7.19 (m, 2H), 7.07 (t, J = 8.1 Hz, 2H), 6.43 (d, J = 7.1 Hz, 1H), 5.06 (s, 2H), 2.78 (s, 6H), 2.21 (s, 3H). | 501.2 |
| | | 3-((4-(4-(4-(2,6-difluorobenzyl)-5- | | | |
| | | oxo-4,5-dihydro-1H-1,2,4-triazol-1- | | | |
| | | yl)phenoxy)-3-methylpyridin-2-yl) | | | |
| | | amino)bicyclo[1.1.1]pentane-1- | | | |
| | | carbonitrile | | | |
| Example 248 Method 61 | ++++ | 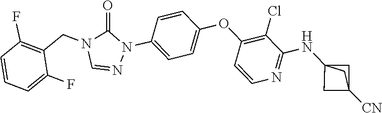 [SMILES: N#CC12CC(Nc3nccc(Oc4ccc(-n5ncn(Cc6c(F)cccc6F)c5=O)cc4)c3Cl)(C1)C2] | yellow solid, yield: 6% | ¹H NMR (400 MHz, CD₃OD) δ 8.01 (s, 1H), 7.93 (d, J = 9.1 Hz, 2H), 7.86 (d, J = 5.7 Hz, 1H), 7.49- 7.42 (m, 1H), 7.14 (d, J = 9.1 Hz, 2H), 7.07 (t, J = 8.1 Hz, 2H), 6.14 (d, J = 5.7 Hz, 1H), 5.05 (s, 2H), 2.64 (s, 6H). | 521.1 |
| | | 3-((3-chloro-4-(4-(4-(2,6-difluoro- | | | |
| | | benzyl)-5-oxo-4,5-dihydro-1H-1,2,4- | | | |
| | | triazol-1-yl)phenoxy)pyridin-2-yl) | | | |
| | | amino)bicyclo[1.1.1]pentane-1- | | | |
| | | carbonitrile | | | |
| Example 249 Method 61 | +++ | 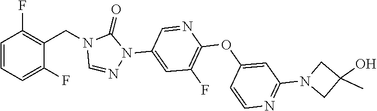 [SMILES: CC1(O)CN(c2cc(Oc3ncc(-n4ncn(Cc5c(F)cccc5F)c4=O)cc3F)ccn2)C1] | off- white solid, yield: 23% | ¹H NMR (400 MHz, CDCl₃) δ 8.68 (d, J = 1.8 Hz, 1H), 8.33 (dd, J = 10.6, 1.8 Hz, 1H), 8.06 (d, J = 6.0 Hz, 1H), 7.64 (s, 1H), 7.44-7.34 (m, 1H), 7.04-6.96 (m, 2H), 6.41 (d, J = 6.0 Hz, 1H), 6.05 (s, 1H), 4.99 (s, 2H), 4.01 (s, 4H), 1.59 (s, 3H). | 485.2 |
| | | 4-(2,6-difluorobenzyl)-2-(5-fluoro- | | | |
| | | 6-((2-(3-hydroxy-3-methylazetidin- | | | |
| | | 1-yl)pyridin-4-yl)oxy)pyridin-3-yl)- | | | |
| | | 2,4-dihydro-3H-1,2,4-triazol-3-one | | | |
| Example 250 Method 61 | ++++ | 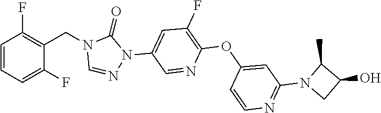 [SMILES: C[C@H]1[C@@H](O)CN1c1cc(Oc2ncc(-n3ncn(Cc4c(F)cccc4F)c3=O)cc2F)ccn1] | off- white solid, yield: 13.4% | ¹H NMR (400 MHz, CDCl₃) δ 8.68 (s, 1H), 8.32 (d, J = 10.5 Hz, 1H), 8.07 (d, J = 5.8 Hz, 1H), 7.63 (s, 1H), 7.44-7.34 (m, 1H), 7.05- 6.97 (m, 2H), 6.37 (d, J = 5.8 Hz, 1H), 6.06 (s, 1H), 4.99 (s, 2H), 4.69 (s, 1H), 4.52 (s, 1H), 4.24 (s, 1H), 3.94 (s, 1H), 1.45 (d, J = 6.4 | 485.2 |
| | | 4-(2,6-difluorobenzyl)-2-(5-fluoro- | | Hz, 4H). | |
| | | 6-((2-((2S,3S)-3-hydroxy-2-methyl- | | | |
| | | azetidin-1-yl)pyridin-4-yl)oxy) | | | |
| | | pyridin-3-yl)-2,4-dihydro-3H-1,2,4- | | | |
| | | triazol-3-one | | | |
| Example 251 Method 61 | ++++ | 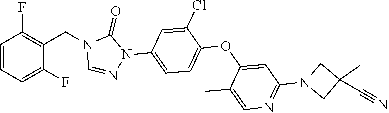 [SMILES: Cc1cnc(N2CC(C)(C#N)C2)cc1Oc1ccc(-n2ncn(Cc3c(F)cccc3F)c2=O)cc1Cl] | off- white solid, yield: 13.4% | ¹H NMR (400 MHz, CDCl₃) δ 8.04 (d, J = 8.6 Hz, 1H), 7.92 (s, 1H), 7.62 (s, 1H), 7.38 (m, 1H), 7.11 (d, J = 9.0 Hz, 2H), 7.04-6.96 (m, 2H), 5.48 (s, 1H), 5.00 (s, 2H), 4.33 (s, 2H), 3.86 (s, 2H), 2.21 (s, 3H), 1.70 (s, 3H). | 522.9 |
| | | 1-(4-(2-chloro-4-(4-(2,6-difluoro- | | | |
| | | benzyl)-5-oxo-4,5-dihydro-1H-1,2,4- | | | |
| | | triazol-1-yl)phenoxy)-5-methylpyridin- | | | |
| | | 2-yl)-3-methylazetidine-3-carbonitrile | | | |
| Example 252 Method 61 | +++ | 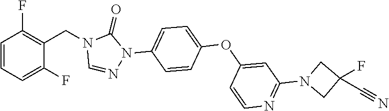 [SMILES: N#CC1(F)CN(c2cc(Oc3ccc(-n4ncn(Cc5c(F)cccc5F)c4=O)cc3)ccn2)C1] | | ¹H NMR (400 MHz, CDCl₃) δ 8.03 (dd, J = 10.4, 3.8 Hz, 3H), 7.61 (s, 1H), 7.37 (d, J = 8.4 Hz, 1H), 7.15- 7.09 (m, 2H), 7.05-6.94 (m, 2H), 6.40 (dd, J = 5.9, 2.0 Hz, 1H), 5.80 (d, J = 2.1 Hz, 1H), 5.00 (s, 2H), 4.57 (s, 2H), 4.39 (dd, J = 21.3, 10.2 Hz, 2H). | 479.1 |
| | | 1-(4-(4-(4-(2,6-difluorobenzyl)-5- | | | |
| | | oxo-4,5-dihydro-1H-1,2,4-triazol-1- | | | |
| | | yl)phenoxy)pyridin-2-yl)-3-fluoro- | | | |
| | | azetidine-3-carbonitrile | | | |
| Example 253 Method 61 | ++++ | 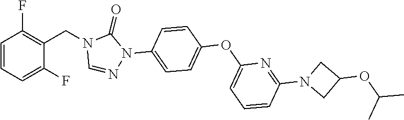 [SMILES: CC(C)OC1CN(c2cccc(Oc3ccc(-n4ncn(Cc5c(F)cccc5F)c4=O)cc3)n2)C1] | | ¹H NMR (400 MHz,CDCl₃) δ 8.04- 7.90 (m, 3H), 7.58 (s, 1H), 7.43- 7.30 (m, 1H), 7.13-7.04 (m, 2H), 6.97 (dd, J = 8.3, 7.5 Hz, 2H), 6.21 (dd, J = 5.8, 2.2 Hz, 1H), 5.73 (d, J = 2.2 Hz, 1H), 4.97 (s, 2H), 4.53- 4.34 (m, 1H), 4.23-4.06 (m, 2H), 3.80 (dd, J = 9.1, 4.8 Hz, 2H), 3.71- | 494.0 |
| | | 4-(2,6-difluorobenzyl)-2-(4-((6-(3- | | 3.54 (m, 1H), 1.16-1.13 (m, 6H). | |
| | | isopropoxyazetidin-1-yl)pyridin-2- | | | |
| | | yl)oxy)phenyl)-2,4-dihydro-3H- | | | |
| | | 1,2,4-triazol-3-one | | | |
| Example 254 Method 61 | +++ | 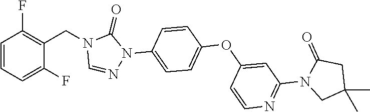 [SMILES: CC1(C)CC(=O)N(c2cc(Oc3ccc(-n4ncn(Cc5c(F)cccc5F)c4=O)cc3)ccn2)C1] | | ¹H NMR (400 MHz, CDCl₃) δ 8.17 (d, J = 5.8 Hz, 1H), 8.05- 7.99 (m, 3H), 7.57 (s, 1H), 7.36 (t, J = 8.4 Hz, 1H), 7.16-7.10 (m, 2H), 6.97 (dd, J = 8.4, 7.5 Hz, 2H), 6.58 (dd, J = 5.8, 2.3 Hz, 1H), 4.97 (s, 2H), 3.82 (s, 2H), 2.43 (s, 2H), 1.20 (s, 6H). | 492.2 |
| | | 4-(2,6-difluorobenzyl)-2-(4-((2- | | | |
| | | (4,4-dimethyl-2-oxopyrrolidin-1-yl) | | | |
| | | pyridin-4-yl)oxy)phenyl)-2,4-dihydro- | | | |
| | | 3H-1,2,4-triazol-3-one | | | |
| Example 255 Method 61 | +++ | 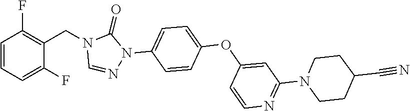 [SMILES: N#CC1CCN(c2cc(Oc3ccc(-n4ncn(Cc5c(F)cccc5F)c4=O)cc3)ccn2)CC1] | | ¹H NMR (400 MHz, CDCl₃) δ 8.05 (d, J = 5.8 Hz, 1H), 8.00 (d, J = 8.9 Hz, 2H), 7.60 (s, 1H), 7.44-7.31 (m, 1H), 7.12 (d, J = 9.0 Hz, 2H), 6.99 (t, J = 7.9 Hz, 2H), 6.26 (d, J = 4.6 Hz, 1H), 6.14 (d, J = 1.5 Hz, 1H), 4.99 (s, 2H), 3.86-3.71 (m, 2H), 3.42 (s, 2H), 2.90-2.79 (m, | 489.2 |
| | | 1-(4-(4-(4-(2,6-difluorobenzyl)-5- | | 1H), 2.01-1.86 (m, 4H). | |
| | | oxo-4,5-dihydro-1H-1,2,4-triazol-1- | | | |
| | | yl)phenoxy)pyridin-2-yl)piperidine- | | | |
| | | 4-carbonitrile | | | |
| Example 256 Method 61 | ++++ | 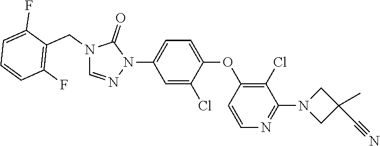 [SMILES: CC1(C#N)CN(c2nccc(Oc3ccc(-n4ncn(Cc5c(F)cccc5F)c4=O)cc3Cl)c2Cl)C1] | | ¹H NMR (400 MHz, CDCl₃) δ 8.20 (d, J = 2.5 Hz, 1H), 7.98 (dd, J = 8.9, 2.6 Hz, 1H), 7.87 (d, J = 5.6 Hz, 1H), 7.62 (s, 1H), 7.40-7.35 (m, 1H), 7.17 (d, J = 8.9 Hz, 1H), 7.02-6.96 (m, 2H), 6.01 (d, J = 5.6 Hz, 1H), 4.99 (s, 2H), 4.62 (d, J = 8.5 Hz, 2H), 4.18 (d, J = 8.5 Hz, 2H), 1.78 (s, 3H). | 543.1 |
| | | 1-(3-chloro-4-(2-chloro-4-(4-(2,6- | | | |
| | | difluorobenzyl)-5-oxo-4,5-dihydro- | | | |
| | | 1H-1,2,4-triazol-1-yl)phenoxy)pyridin- | | | |
| | | 2-yl)-3-methylazetidine-3-carbonitrile | | | |
| Example 257 Method 61 | ++++ | 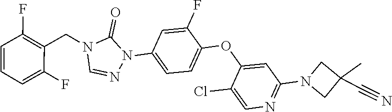 [SMILES: CC1(C#N)CN(c2cc(Oc3ccc(-n4ncn(Cc5c(F)cccc5F)c4=O)cc3F)c(Cl)cn2)C1] | | ¹H NMR (400 MHz, DMSO-d₆) δ 8.40 (s, 1H), 8.18 (s, 1H), 7.94 (dd, J = 12.4, 2.5 Hz, 1H), 7.82-7.75 (m, 1H), 7.52-7.41 (m, 2H), 7.20- 7.12 (m, 2H), 5.74 (s, 1H), 5.00 (s, 2H), 4.15 (d, J = 8.3 Hz, 2H), 3.81 (d, J = 8.3 Hz, 2H), 1.57 (s, 3H). | 527.1 |
| | | 1-(5-chloro-4-(4-(4-(2,6-difluoro- | | | |
| | | benzyl)-5-oxo-4,5-dihydro-1H-1,2,4- | | | |
| | | triazol-1-yl)-2-fluorophenoxy)pyridin- | | | |
| | | 2-yl)-3-methylazetidine-3-carbonitrile | | | |
| Example 258 Method 61 Single unknown stereo- isomer | ++++ | 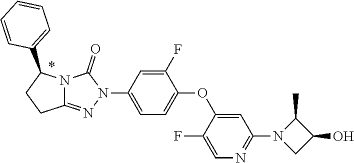 [SMILES: C[C@H]1[C@@H](O)CN1c1cc(Oc2ccc(-n3nc4n(c3=O)[C@H](c3ccccc3)CC4)cc2F)c(F)cn1] | white solid, yield: 25% | ¹H NMR (400 MHz, CDCl₃) δ 8.03-7.91 (m, 2H), 7.84 (d, J = 8.4 Hz, 1H), 7.45-7.26 (m, 5H), 7.21 (t, J = 8.7 Hz, 1H), 5.55 (d, J = 5.6 Hz, 1H), 5.30 (dd, J = 7.4, 4.2 Hz, 1H), 4.70-4.60 (m, 1H), 4.50-4.35 (m, 1H), 4.20-4.05 (m, 1H), 3.95- 3.80 (m, 1H), 3.15-2.88 (m, 3H), 2.62-2.48 (m, 1H), 1.36 (d, J = 6.3 Hz, 3H). | 492.1 |
| | | (S)-2-(3-fluoro-4-((5-fluoro-2-((2S,3S)- | | | |
| | | 3-hydroxy-2-methylazetidin-1- | | | |
| | | yl)pyridin-4-yl)oxy)phenyl)-5-phenyl- | | | |
| | | 2,5,6,7-tetrahydro-3H-pyrrolo[2,1- | | | |
| | | c][1,2,4]triazol-3-one | | | |
| Example 259 Method 61 | +++ | 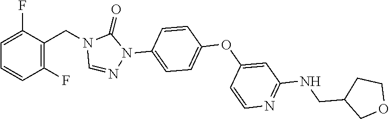 [SMILES: O=c1n(Cc2c(F)cccc2F)cnn1-c1ccc(Oc2ccnc(NCC3CCOC3)c2)cc1] | white solid, | ¹H NMR (400 MHz, CDCl3) δ 8.08 (t, J = 8.0 Hz, 2H), 7.73 (s, 1H), 7.62 (s, 1H), 7.44-7.35 (m, 1H), 7.18-7.12 (m, 2H), 7.04-6.96 (m, 2H), 6.33 (d, J = 6.5 Hz, 1H), 5.90 (d, J = 2.2 Hz, 1H), 5.00 (s, 2H), 3.94-3.79 (m, 2H), 3.79- 3.68 (m, 1H), 3.61-3.52 (m, 1H), | 480.5 |
| | | 4-(2,6-difluorobenzyl)-2-(4-((2- | | 3.18-2.99 (m, 2H), 2.65-2.56 | |
| | | (((tetrahydrofuran-3-yl)methyl)amino) | | (m, 1H), 2.23-2.08 (m, 1H), 1.69- | |
| | | pyridin-4-yl)oxy)phenyl)-2,4-dihydro- | | 1.64 (m, 1H). | |
| | | 3H-1,2,4-triazol-3-one | | | |
| Example 260 Method 61 | ++++ | 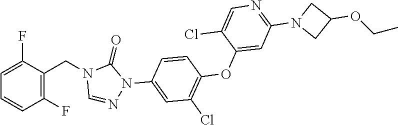 [SMILES: CCOC1CN(c2cc(Oc3ccc(-n4ncn(Cc5c(F)cccc5F)c4=O)cc3Cl)c(Cl)cn2)C1] | white solid, yield: 43.3% | ¹H NMR (400 MHz, CDCl₃) δ 8.23 (d, J = 2.4 Hz, 1H), 7.978-8.043 (s, 1H), 8.01 (m, 1H), 7.63 (s, 1H), 7.45-7.33 (m, 1H), 7.21 (d, J = 8.8 Hz, 1H), 7.03-6.95 (m, 2H), 5.34 (s, 1H), 5.00 (s, 2H), 4.35 (s, 1H), 4.21 (s, 2H), 3.89 (s, 2H), 3.48-3.38 (m, 2H), 1.19 (t, J = 7.2 | 548.4 |
| | | 2-(3-chloro-4-((5-chloro-2-(3-ethoxy- | | Hz, 3H). | |
| | | azetidin-1-yl)pyridin-4-yl)oxy) | | | |
| | | phenyl)-4-(2,6-difluorobenzyl)-2,4- | | | |
| | | dihydro-3H-1,2,4-triazol-3-one | | | |
| Example 261 Method 61 | +++ | 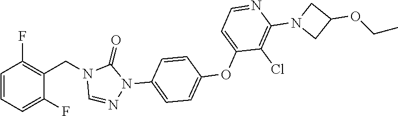 [SMILES: CCOC1CN(c2nccc(Oc3ccc(-n4ncn(Cc5c(F)cccc5F)c4=O)cc3)c2Cl)C1] | white solid, yield: 20.3% | ¹H NMR (400 MHz, CDCl3) δ 8.19-8.03 (m, 2H), 7.68-7.49 (m, 2H), 7.45-7.33 (m, 1H), 7.19- 7.07 (m, 4H), 7.03-6.95 (m, 4H), 6.59 (d, J = 5.6 Hz, 1H), 5.00 (d, J = 7.6 Hz, 2H), 4.67 (d, J = 7.8 Hz, 1H), 4.45-4.24 (m, 1H), 3.56- 3.49 (m, 1H), 2.42-1.95 (m, 3H). | 514.4 |
| | | 2-(4-((3-chloro-2-(3-ethoxyazetidin- | | | |
| | | 1-yl)pyridin-4-yl)oxy)phenyl)-4-(2,6- | | | |
| | | difluorobenzyl)-2,4-dihydro-3H- | | | |
| | | 1,2,4-triazol-3-one | | | |
| Example 262 Method 61 | ++++ | 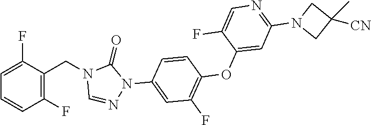 [SMILES: CC1(C#N)CN(c2cc(Oc3ccc(-n4ncn(Cc5c(F)cccc5F)c4=O)cc3F)c(F)cn2)C1] | yellow solid, yield: 22.3% | ¹H NMR (400 MHz, CDCl₃) δ 8.07-8.00 (m, 2H), 7.92 (d, J = 5.2 Hz, 1H), 7.64 (s, 1H), 7.44-7.35 (m, 1H), 7.24 (s, 1H), 7.05-6.97 (m, 2H), 5.51 (d, J = 6.4 Hz, 1H), 5.00 (s, 2H), 4.48 (s, 2H), 4.10 (s, 2H), 1.72 (s, 3H). | 511.5 |
| | | 1-(4-(4-(4-(2,6-difluorobenzyl)-5- | | | |
| | | oxo-4,5-dihydro-1H-1,2,4-triazol-1- | | | |
| | | yl)-2-fluorophenoxy)-5-fluoropyridin- | | | |
| | | 2-yl)-3-methylazetidine-3-carbonitrile | | | |
| Example 263 Method 61 | ++++ | 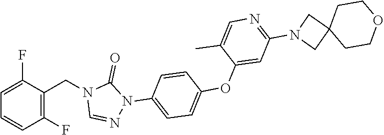 [SMILES: Cc1cnc(N2CC3(CCOCC3)C2)cc1Oc1ccc(-n2ncn(Cc3c(F)cccc3F)c2=O)cc1] | yellow solid, yield: 25% | ¹H NMR (400 MHz, CDCl₃) δ 8.09- 7.94 (m, 2H), 7.89 (s, 1H), 7.60 (d, J = 11.2 Hz, 1H), 7.43-7.33 (m, 1H), 7.15-7.06 (m, 2H), 7.03- 6.97 (m, 3H), 5.00 (s, 2H), 3.77- 3.59 (m, 4H), 2.18 (s, 3H), 1.90- 1.70 (m, 4H), 1.70 (m, 4H). | 520.2 |
| | | 4-(2,6-difluorobenzyl)-2-(4-((5- | | | |
| | | methyl-2-(7-oxa-2-azaspiro[3.5]nonan- | | | |
| | | 2-yl)pyridin-4-yl)oxy)phenyl)-2,4- | | | |
| | | dihydro-3H-1,2,4-triazol-3-one | | | |
| Example 264 Method 61 | ++++ | 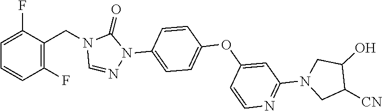 [SMILES: N#CC1CN(c2cc(Oc3ccc(-n4ncn(Cc5c(F)cccc5F)c4=O)cc3)ccn2)CC1O] | white solid, yield: 14.7% | ¹H NMR (400 MHz, CDCl₃) δ 8.09-8.02 (m, 3H), 7.61 (s, 1H), 7.41-7.33 (m, 1H), 7.18-7.10 (m, 2H), 7.04-6.95 (m, 2H), 6.36 (dd, J = 6.8, 1.6 Hz, 1H), 5.88-5.83 (m, 1H), 4.99 (s, 2H), 4.80-4.70 (m, 1H), 4.00-3.60 (m, 4H), 3.56-3.21 (m, 1H). | 491.1 |
| | | 1-(4-(4-(4-(2,6-difluorobenzyl)-5- | | | |
| | | oxo-4,5-dihydro-1H-1,2,4-triazol-1- | | | |
| | | yl)phenoxy)pyridin-2-yl)-4-hydroxy- | | | |
| | | pyrrolidine-3-carbonitrile | | | |
| Example 265 Method 61 | +++ | 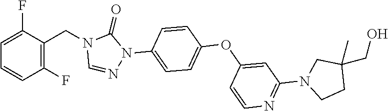 [SMILES: CC1(CO)CCN(c2cc(Oc3ccc(-n4ncn(Cc5c(F)cccc5F)c4=O)cc3)ccn2)C1] | white solid, yield: 15% | ¹H NMR (400 MHz, CDCl₃) δ 8.09-8.02 (m, 3H), 7.61 (s, 1H), 7.41-7.33 (m, 1H), 7.20-7.10 (m, 2H), 7.06-6.84 (m, 2H), 6.31 (dd, J = 6.8, 1.6 Hz, 1H), 5.81 (d, J = 2.0 Hz, 1H), 4.99 (s, 2H), 3.78- 3.37 (m, 4H), 3.17 (d, J = 10.4 Hz, 2H), 1.83-1.64 (m, 2H), 1.14 (s, | 494.1 |
| | | 4-(2,6-difluorobenzyl)-2-(4-((2-(3- | | 3H). | |
| | | (hydroxymethyl)-3-methylpyrrolidin- | | | |
| | | 1-yl)pyridin-4-yl)oxy)phenyl)-2,4- | | | |
| | | dihydro-3H-1,2,4-triazol-3-one | | | |
| Example 266 Method 61 | ++++ | 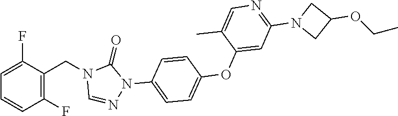 [SMILES: CCOC1CN(c2cc(Oc3ccc(-n4ncn(Cc5c(F)cccc5F)c4=O)cc3)c(C)cn2)C1] | white solid, yield: 47.6% | ¹H NMR (400 MHz, CDCl₃) δ 8.12 (d, J = 8.8 Hz, 2H), 7.86 (s, 1H), 7.64 (s, 1H), 7.43-7.36 (m, 1H), 7.15 (d, J = 8.8 Hz, 1H), 7.07-6.99 (m, 2H), 5.38 (s, 1H), 5.01 (s, 2H), 4.35 (s, 2H), 4.12 (q, J = 7.2 Hz, 1H), 3.99 (s, 3H), 3.43 (q, J = 6.8 Hz, 2H), 2.23 (s, 3H), 1.18 (t, J = | 494.1 |
| | | 4-(2,6-difluorobenzyl)-2-(4-((2-(3- | | 6.8 Hz, 3H). | |
| | | ethoxyazetidin-1-yl)-5-methylpyridin- | | | |
| | | 4-yl)oxy)phenyl)-2,4-dihydro-3H- | | | |
| | | 1,2,4-triazol-3-one | | | |
| Example 267 Method 61 | ++++ | 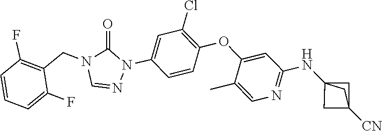 [SMILES: Cc1cnc(NC23CC(C#N)(C2)C3)cc1Oc1ccc(-n2ncn(Cc3c(F)cccc3F)c2=O)cc1Cl] | | ¹H NMR (400 MHz, CDCl₃) δ 8.36 (d, J = 2.4 Hz, 1H), 8.16 (dd, J = 9.2, 2.4 Hz, 1H), 7.77-7.62 (m, 2H), 7.59-7.34 (m, 2H), 7.24 (d, J = 9.2 Hz, 1H), 7.05-6.96 (m, 2H), 5.01 (s, 2H), 2.36 (s, 6H), 2.26 (s, 3H). | 535.2 |
| | | 3-((4-(2-chloro-4-(4-(2,6-difluoro- | | | |
| | | benzyl)-5-oxo-4,5-dihydro-1H-1,2,4- | | | |
| | | triazol-1-yl)phenoxy)-5-methyl- | | | |
| | | pyridin-2-yl)amino)bicyclo[1.1.1] | | | |
| | | pentane-1-carbonitrile | | | |
| Example 268 Method 61 | ++++ | 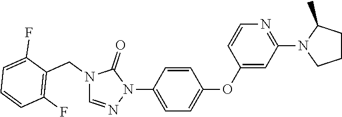 [SMILES: C[C@H]1CCCN1c1cc(Oc2ccc(-n3ncn(Cc4c(F)cccc4F)c3=O)cc2)ccn1] | white solid, yield: 19.5% | ¹H NMR (400 MHz, CDCl₃) δ 8.09 (m, 3H), 7.63 (s, 1H), 7.42-7.35 (m, 1H), 7.16 (d, J = 9.0 Hz, 2H), 7.00 (m, 2H), 6.42 (d, J = 5.2 Hz, 1H), 5.89 (s, 1H), 5.00 (s, 2H), 3.77-3.36 (m, 2H), 2.12 (d, J = 5.6 Hz, 3H), 1.79 (s, 1H), 1.26 (s, 1H), 1.17 (d, J = 5.6 Hz, 3H). | 464.1 |
| | | (S)-4-(2,6-difluorobenzyl)-2-(4-((2- | | | |
| | | (2-methylpyrrolidin-1-yl)pyridin-4- | | | |
| | | yl)oxy)phenyl)-2,4-dihydro-3H- | | | |
| | | 1,2,4-triazol-3-one | | | |
| Example 269 Method 61 | +++ | 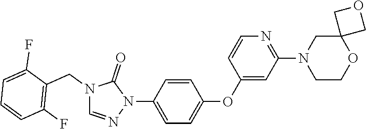 [SMILES: O=c1n(Cc2c(F)cccc2F)cnn1-c1ccc(Oc2ccnc(N3CCOC4(COC4)C3)c2)cc1] | white solid, yield: 1.36% | 1H NMR (400 MHz, CDCl3) δ 8.13 (m, 3H), 7.63 (s, 1H), 7.38 (m, 1H), 7.16 (d, J = 8.8 Hz, 2H), 7.01 (d, J = 7.8 Hz, 2H), 6.49 (s, 1H), 6.23 (s, 1H), 5.00 (s, 2H), 4.63 (d, J = 6.9 Hz, 2H), 4.44 (d, J = 6.9 Hz, 2H), 3.78 (m, 6H). | 508.2 |
| | | 2-(4-((2-(2,5-dioxa-8-azaspiro[3.5] | | | |
| | | nonan-8-yl)pyridin-4-yl)oxy)phenyl)- | | | |
| | | 4-(2,6-difluorobenzyl)-2,4-dihydro- | | | |
| | | 3H-1,2,4-triazol-3-one | | | |
| Example 270 Method 61 | ++++ | 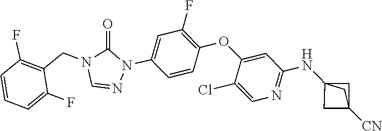 [SMILES: N#CC12CC(Nc3cc(Oc4ccc(-n5ncn(Cc6c(F)cccc6F)c5=O)cc4F)c(Cl)cn3)(C1)C2] | white solid, yield: 8.23% | ¹H NMR (400 MHz, CDCl₃) δ 8.03 (m, 2H), 7.93 (d, J = 9.0 Hz, 1H), 7.64 (s, 1H), 7.42-7.34 (m, 1H), 7.22 (d, J = 8.7 Hz, 1H), 7.00 (m, 2H), 5.71 (s, 1H), 5.00 (s, 2H), 2.44 (s, 6H). | 539.2 |
| | | 3-((5-chloro-4-(4-(4-(2,6-difluoro- | | | |
| | | benzyl)-5-oxo-4,5-dihydro-1H-1,2,4- | | | |
| | | triazol-1-yl)-2-fluorophenoxy)pyridin- | | | |
| | | 2-yl)amino)bicyclo[1.1.1]pentane- | | | |
| | | 1-carbonitrile | | | |
| Example 271 Method 61 | ++++ | 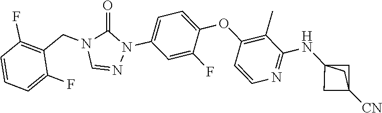 [SMILES: Cc1c(Oc2ccc(-n3ncn(Cc4c(F)cccc4F)c3=O)cc2F)ccnc1NC12CC(C#N)(C1)C2] | white solid, yield: 4.9% | ¹H NMR (400 MHz, CDCl₃) δ 8.20 (s, 1H), 8.03 (d, J = 11.8 Hz, 1H), 7.94 (d, J = 8.9 Hz, 1H), 7.63 (s, 1H), 7.39 (s, 1H), 7.21 (d, J = 8.2 Hz, 1H), 7.02-6.98 (m, 2H), 6.34 (s, 1H), 5.00 (s, 2H), 2.91 (s, 6H), 2.29 (s, 3H). | 519.2 |
| | | 3-((4-(4-(4-(2,6-difluorobenzyl)-5- | | | |
| | | oxo-4,5-dihydro-1H-1,2,4-triazol-1- | | | |
| | | yl)-2-fluorophenoxy)-3-methylpyridin- | | | |
| | | 2-yl)amino)bicyclo[1.1.1]pentane- | | | |
| | | 1-carbonitrile | | | |
| Example 272 Method 61 | ++++ | 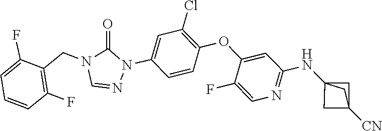 [SMILES: N#CC12CC(Nc3cc(Oc4ccc(-n5ncn(Cc6c(F)cccc6F)c5=O)cc4Cl)c(F)cn3)(C1)C2] | white solid, yield: 1.8% | ¹H NMR (400 MHz, CDCl₃) δ 8.23 (d, J = 2.3 Hz, 1H), 8.09 (d, J = 9.0 Hz, 2H), 7.62 (s, 1H), 7.37 (d, J = 8.3 Hz, 1H), 7.18 (d, J = 9.0 Hz, 2H), 6.99 (d, J = 7.9 Hz, 2H), 6.69 (d, J = 5.9 Hz, 1H), 5.00 (s, 3H), 1.75 (s, 6H). | 539.2 |
| | | 3-((4-(2-chloro-4-(4-(2,6-difluoro- | | | |
| | | benzyl)-5-oxo-4,5-dihydro-1H-1,2,4- | | | |
| | | triazol-1-yl)phenoxy)-5-fluoropyridin- | | | |
| | | 2-yl)amino)bicyclo[1.1.1]pentane- | | | |
| | | 1-carbonitrile | | | |
| Example 273 Method 61 | +++ | 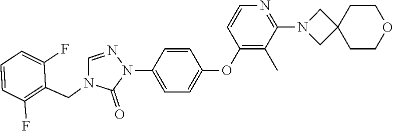 [SMILES: Cc1c(Oc2ccc(-n3ncn(Cc4c(F)cccc4F)c3=O)cc2)ccnc1N1CC2(CCOCC2)C1] | white solid, yield: 38.4% | 1H NMR (400 MHz, CDCl3) δ 8.26 (s, 1H), 7.90-7.73 (m, 3H), 7.49-7.38 (m, 1H), 7.18-7.02 (m, 4H), 6.09 (d, J = 5.6 Hz, 1H), 4.94 (s, 2H), 3.82 (s, 4H), 3.58- 3.44 (m, 4H), 2.02 (d, J = 12.8 Hz, 3H), 1.74-1.64 (m, 4H). | 520.0 |
| | | 4-(2,6-difluorobenzyl)-2-(4-((3- | | | |
| | | methyl-2-(7-oxa-2-azaspiro[3.5]nonan- | | | |
| | | 2-yl)pyridin-4-yl)oxy)phenyl)-2,4- | | | |
| | | dihydro-3H-1,2,4-triazol-3-one | | | |
| Example 274 Method 61 | ++ | 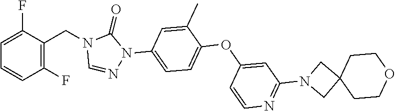 [SMILES: Cc1cc(-n2ncn(Cc3c(F)cccc3F)c2=O)ccc1Oc1ccnc(N2CC3(CCOCC3)C2)c1] | white solid, yield: 38.4% | ¹H NMR (400 MHz, CDCl₃) δ 7.99 (s, 1H), 7.84 (t, J = 4.0 Hz, 2H), 7.75 (dd, J = 8.7, 2.6 Hz, 1H), 7.43 (ddd, J = 8.4, 7.5, 4.2 Hz, 1H), 7.11-6.97 (m, 3H), 6.17 (dd, J = 6.0, 2.2 Hz, 1H), 5.66 (d, J = 2.2 Hz, 1H), 5.02 (s, 2H), 3.64-3.55 | 520.2 |
| | | 2-(4-((2-(7-oxa-2-azaspiro[3.5]nonan- | | (m, 4H), 3.28 (dt, J = 3.3, 1.7 Hz, | |
| | | 2-yl)pyridin-4-yl)oxy)-3-methyl- | | 4H), 2.16 (s, 3H), 1.79-1.71 (m, | |
| | | phenyl)-4-(2,6-difluorobenzyl)-2,4- | | 4H). | |
| | | dihydro-3H-1,2,4-triazol-3-one | | | |
| Example 275 Method 61 | +++ | 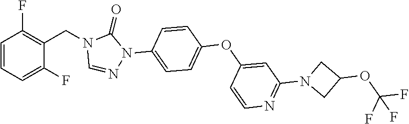 [SMILES: O=c1n(Cc2c(F)cccc2F)cnn1-c1ccc(Oc2ccnc(N3CC(OC(F)(F)F)C3)c2)cc1] | white solid, yield: 50% | ¹H NMR (400 MHz, CDCl₃) δ 8.11- 7.98 (m, 3H), 7.61 (s, 1H), 7.44- 7.31 (m, 1H), 7.18-7.09 (m, 2H), 7.05-6.94 (m, 2H), 6.34 (dd, J = 6.1, 2.1 Hz, 1H), 5.74 (d, J = 2.1 Hz, 1H), 5.09-5.03 (m, 1H), 4.99 (s, 2H), 4.45-4.31 (m, 2H), 4.17- 4.09 (m, 2H). | 520.1 |
| | | 4-(2,6-difluorobenzyl)-2-(4-((2-(3- | | | |
| | | (trifluoromethoxy)azetidin-1-yl)pyridin- | | | |
| | | 4-yl)oxy)phenyl)-2,4-dihydro-3H- | | | |
| | | 1,2,4-triazol-3-one | | | |
| Example 276 Method 61 | ++++ | 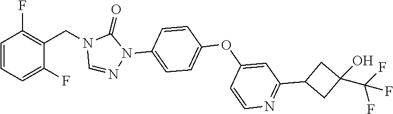 [SMILES: O=c1n(Cc2c(F)cccc2F)cnn1-c1ccc(Oc2ccnc(C3CC(O)(C(F)(F)F)C3)c2)cc1] | white solid, yield: 41% | ¹H NMR (400 MHz, CDCl₃) δ 8.05- 7.93 (m, 3H), 7.60 (s, 1H), 7.37 (ddd, J = 8.5, 6.6, 1.9 Hz, 1H), 7.16-7.08 (m, 2H), 7.05-6.94 (m, 2H), 6.31 (dd, J = 6.0, 2.1 Hz, 1H), 5.74 (d, J = 2.1 Hz, 1H), 4.99 (s, 2H), 4.27 (d, J = 9.5 Hz, 2H), 4.02 (d, J = 9.5 Hz, 2H). | 520.1 |
| | | 4-(2,6-difluorobenzyl)-2-(4-((2-(3- | | | |
| | | hydroxy-3-(trifluoromethyl)azetidin- | | | |
| | | 1-yl)pyridin-4-yl)oxy)phenyl)-2,4- | | | |
| | | dihydro-3H-1,2,4-triazol-3-one | | | |
| Example 277 Method 61 | +++ | 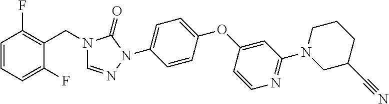 [SMILES: N#CC1CCCN(c2cc(Oc3ccc(-n4ncn(Cc5c(F)cccc5F)c4=O)cc3)ccn2)C1] | white solid, yield: 11.9% | ¹H NMR (400 MHz, CDCl₃) δ 8.07- 7.97 (m, 3H), 7.60 (s, 1H), 7.37 (tt, J = 8.4, 6.5 Hz, 1H), 7.15- 7.09 (m, 2H), 7.04-6.95 (m, 2H), 6.26 (d, J = 5.7 Hz, 1H), 6.18 (d, J = 2.0 Hz, 1H), 4.99 (s, 2H), 4.16 (d, J = 12.7 Hz, 1H), 3.62 (dt, J = | 489.1 |
| | | 1-(4-(4-(4-(2,6-difluorobenzyl)-5- | | 9.4, 4.5 Hz, 1H), 3.52 (s, 1H), 3.27 | |
| | | oxo-4,5-dihydro-1H-1,2,4-triazol-1- | | (s, 1H), 2.84-2.74 (m, 1H), 2.13- | |
| | | yl)phenoxy)pyridin-2-yl)piperidine- | | 2.04 (m, 1H), 1.96-1.79 (m, 2H), | |
| | | 3-carbonitrile | | 1.66-1.51 (m, 1H). | |
| Example 278 Method 61 | +++ | 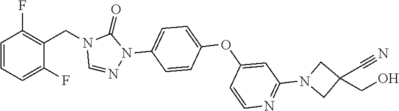 [SMILES: N#CC1(CO)CN(c2cc(Oc3ccc(-n4ncn(Cc5c(F)cccc5F)c4=O)cc3)ccn2)C1] | white solid, yield: 5% | ¹H NMR (400 MHz, CDCl₃) δ 8.08 (d, J = 9.1 Hz, 2H), 7.95 (d, J = 6.6 Hz, 1H), 7.62 (s, 1H), 7.43-7.33 (m, 1H), 7.14 (d, J = 9.0 Hz, 2H), 7.00 (t, J = 7.9 Hz, 2H), 6.45 (dd, J = 6.7, 2.1 Hz, 1H), 5.70 (d, J = 2.1 Hz, 1H), 5.00 (s, 2H), 4.42 (s, 4H), 3.98 (s, 2H). | 491.1 |
| | | 1-(4-(4-(4-(2,6-difluorobenzyl)-5- | | | |
| | | oxo-4,5-dihydro-1H-1,2,4-triazol-1- | | | |
| | | yl)phenoxy)pyridin-2-yl)-3-(hydroxy- | | | |
| | | methyl)azetidine-3-carbonitrile | | | |
| Example 279 Method 61 | +++ | 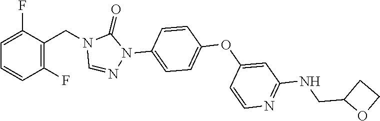 [SMILES: O=c1n(Cc2c(F)cccc2F)cnn1-c1ccc(Oc2ccnc(NCC3CCO3)c2)cc1] | white solid, yield: 13.1% | ¹H NMR (400 MHz, CDCl₃) δ 8.06- 7.95 (m, 2H), 7.89 (d, J = 6.1 Hz, 1H), 7.60 (s, 1H), 7.40-7.33 (m, 1H), 7.18-7.09 (m, 2H), 7.04- 6.95 (m, 2H), 6.25 (dd, J = 6.1, 2.2 Hz, 1H), 5.98 (d, J = 2.1 Hz, 1H), 5.61 (s, 1H), 5.05-4.96 (m, 3H), 4.66 (dd, J = 8.0, 1.9 Hz, 1H), 4.58- | 466.1 |
| | | 4-(2,6-difluorobenzyl)-2-(4-((2- | | 4.48 (m, 1H), 3.58-3.40 (m, | |
| | | ((oxetan-2-ylmethyl)amino)pyridin-4- | | 2H), 2.72-2.47 (m, 2H). | |
| | | yl)oxy)phenyl)-2,4-dihydro-3H-1,2,4- | | | |
| | | triazol-3-one | | | |
| Example 280 Method 61 | +++ | 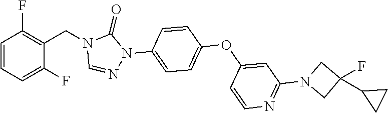 [SMILES: O=c1n(Cc2c(F)cccc2F)cnn1-c1ccc(Oc2ccnc(N3CC(F)(C4CC4)C3)c2)cc1] | white solid | ¹H NMR (400 MHz, DMSO-d₆) δ 8.32 (s, 1H), 7.99-7.97 (m, 1H), 7.94-7.90 (m, 2H), 7.52-7.45 (m, 1H), 7.25-7.21 (m, 2H), 7.19-7.16 (m, 2H), 6.27-6.24 (m, 1H), 5.93-5.90 (m, 1H), 4.99 (s, 2H), 3.92-3.90 (m, 2H), 3.86-3.80 (m, 2H), 1.39-1.36 (m, 1H), | 494.2 |
| | | 2-(4-((2-(3-cyclopropyl-3-fluoro- | | 0.60-0.58 (m, 2H), 0.44-0.41 (m, | |
| | | azetidin-1-yl)pyridin-4-yl)oxy) | | 2H). | |
| | | phenyl)-4-(2,6-difluorobenzyl)-2,4- | | | |
| | | dihydro-3H-1,2,4-triazol-3-one | | | |
| Example 281 Method 61 | ++++ | 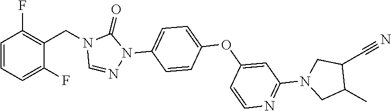 [SMILES: CC1CN(c2cc(Oc3ccc(-n4ncn(Cc5c(F)cccc5F)c4=O)cc3)ccn2)CC1C#N] | white solid, yield: 21.5% | ¹H NMR (400 MHz, CDCl₃) δ 8.06 (d, J = 6.7 Hz, 3H), 7.62 (s, 1H), 7.37 (d, J = 8.4 Hz, 1H), 7.15 (d, J = 9.0 Hz, 2H), 7.03-6.96 (m, 2H), 6.39 (s, 1H), 5.83 (d, J = 2.0 Hz, 1H), 5.00 (s, 2H), 4.04-3.64 (m, 4H), 3.30 (s, 1H), 2.66 (s, 1H), 1.33 (d, J = 6.7 Hz, 3H). | 489.1 |
| | | 1-(4-(4-(4-(2,6-difluorobenzyl)-5- | | | |
| | | oxo-4,5-dihydro-1H-1,2,4-triazol-1- | | | |
| | | yl)phenoxy)pyridin-2-yl)-4-methyl- | | | |
| | | pyrrolidine-3-carbonitrile | | | |
| Example 282 Method 61 | ++++ | 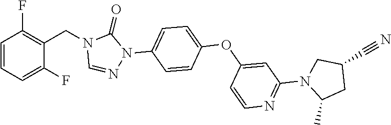 [SMILES: C[C@H]1C[C@@H](C#N)CN1c1cc(Oc2ccc(-n3ncn(Cc4c(F)cccc4F)c3=O)cc2)ccn1] | white solid, yield: 4.8% | ¹H NMR (400 MHz, CDCl3) δ 8.02 (ddd, J = 18.3, 8.5, 5.6 Hz, 3H), 7.64-7.58 (m, 1H), 7.41-7.31 (m, 1H), 7.20-7.09 (m, 2H), 7.04- 6.93 (m, 2H), 6.28-6.20 (m, 1H), 5.88 (dd, J = 8.3, 2.0 Hz, 1H), 4.99 (s, 2H), 3.91-3.59 (m, 2H), 3.36-3.08 (m, 1H), 2.59-2.37 (m, 1H), 2.33-2.03 (m, 2H), 1.39 | 488.9 |
| | | (3R,5S)-1-(4-(4-(4-(2,6-difluoro- | | (t, J = 6.4 Hz, 2H), 1.20 (d, J = 6.3 | |
| | | benzyl)-5-oxo-4,5-dihydro-1H-1,2,4- | | Hz, 1H). | |
| | | triazol-1-yl)phenoxy)pyridin-2-yl)-5- | | | |
| | | methylpyrrolidine-3-carbonitrile | | | |
| Example 283 Method 61 | ++++ | 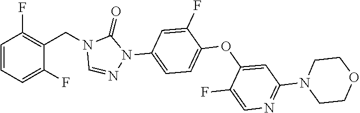 [SMILES: O=c1n(Cc2c(F)cccc2F)cnn1-c1ccc(Oc2cc(N3CCOCC3)ncc2F)c(F)c1] | | ¹H NMR (400 MHz, DMSO-d₆) δ 8.39 (s, 1H), 8.19 (d, J = 2.6 Hz, 1H), 7.95-7.92 (m, 1H), 7.80-7.75 (m, 1H), 7.53-7.43 (m, 2H), 7.20- 7.13 (m, 2H), 6.24 (d, J = 5.6 Hz, 1H), 5.00 (s, 2H), 3.64-3.58 (m, 4H), 3.29-3.24 (m, 4H). | 502.0 |
| | | 4-(2,6-difluorobenzyl)-2-(3-fluoro- | | | |
| | | 4-((5-fluoro-2-morpholinopyridin-4- | | | |
| | | yl)oxy)phenyl)-2,4-dihydro-3H-1,2,4- | | | |
| | | triazol-3-one | | | |
| Example 284 Method 61 | ++++ | 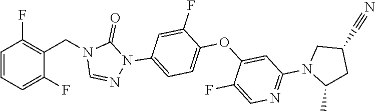 [SMILES: C[C@H]1C[C@@H](C#N)CN1c1cc(Oc2ccc(-n3ncn(Cc4c(F)cccc4F)c3=O)cc2F)c(F)cn1] | white solid, yield: 12.9% | ¹H NMR (400 MHz, CDCl₃) δ 8.11- 7.97 (m, 2H), 7.88 (dd, J = 9.0, 1.5 Hz, 1H), 7.63 (s, 1H), 7.38 (td, J = 8.4, 4.3 Hz, 1H), 7.23 (d, J = 8.7 Hz, 1H), 7.01 (dd, J = 13.6, 5.6 Hz, 2H), 5.67 (dd, J = 10.8, 5.4 Hz, 1H), 4.99 (s, 2H), 4.13 (dd, J = 25.6, 18.6 Hz, 1H), 3.82-3.52 (m, | 525.1 |
| | | (3R,5S)-1-(4-(4-(4-(2,6-difluoro- | | 2H), 3.13 (s, 1H), 2.52 (dd, J = 7.8, | |
| | | benzyl)-5-oxo-4,5-dihydro-1H-1,2,4- | | 5.2 Hz, 1H), 2.14-1.98 (m, 1H), | |
| | | triazol-1-yl)-2-fluorophenoxy)-5- | | 1.32 (d, J = 6.3 Hz, 2H), 1.15 (d, | |
| | | fluoropyridin-2-yl)-5-methylpyrrolidine- | | J = 6.3 Hz, 1H). | |
| | | 3-carbonitrile | | | |
| Example 285 Method 61 | ++++ | 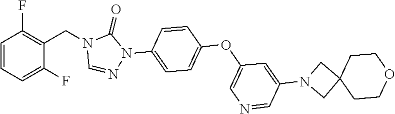 [SMILES: O=c1n(Cc2c(F)cccc2F)cnn1-c1ccc(Oc2cncc(N3CC4(CCOCC4)C3)c2)cc1] | white solid, yield: 5% | 1H NMR (400 MHz, CDCl3) δ 7.94 (d, J = 9.1 Hz, 2H), 7.78 (s, 1H), 7.59 (m, 2H), 7.37 (m, J = 14.9, 8.4, 6.5 Hz, 2H), 7.07 (d, J = 9.1 Hz, 2H), 6.99 (dd, J = 9.6, 6.2 Hz, 2H), 4.99 (s, 2H), 3.65 (d, J = 7.5 Hz, 8H), 1.85-1.81 (m, 4H). | 506.0 |
| | | 2-(4-((5-(7-oxa-2-azaspiro[3.5]nonan- | | | |
| | | 2-yl)pyridin-3-yl)oxy)phenyl)-4- | | | |
| | | (2,6-difluorobenzyl)-2,4-dihydro-3H- | | | |
| | | 1,2,4-triazol-3-one | | | |
| Example 286 Method 61 | +++ | 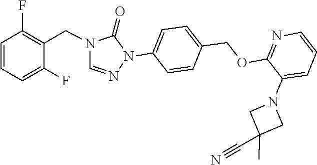 [SMILES: CC1(C#N)CN(c2cccnc2OCc2ccc(-n3ncn(Cc4c(F)cccc4F)c3=O)cc2)C1] | white solid, yield: | ¹H NMR (400 MHz, DMSO-d₆) δ 8.31 (s, 1H), 7.88 (d, J = 8.6 Hz, 2H), 7.62-7.59 (m, 1H), 7.53 (d, J = 8.6 Hz, 2H), 7.49-7.45 (m, 1H), 7.18-7.16 (m, 2H), 6.89-6.86 (m, 1H), 6.79-6.75 (m, 1H), 5.32 (s, 2H), 4.98 (s, 2H), 4.15 (d, J = 7.9 Hz, 2H), 3.82 (d, J = 7.9 Hz, 2H), 1.59 (s, 3H). | 489.2 |
| | | 1-(2-((4-(4-(2,6-difluorobenzyl)-5- | | | |
| | | oxo-4,5-dihydro-1H-1,2,4-triazol-1- | | | |
| | | yl)benzyl)oxy)pyridin-3-yl)-3- | | | |
| | | methylazetidine-3-carbonitrile | | | |
| Example 287 Method 61 | ++++ | 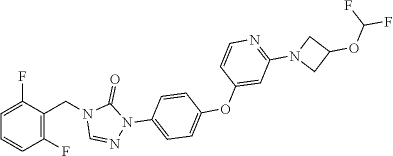 [SMILES: O=c1n(Cc2c(F)cccc2F)cnn1-c1ccc(Oc2ccnc(N3CC(OC(F)F)C3)c2)cc1] | yellow solid, yield: 15% | ¹H NMR (400 MHz, MeOH-d₄) δ 8.16-7.88 (m, 4H), 7.47-7.49 (m, 1H), 7.21 (d, J = 8.9 Hz, 2H), 7.10 (t, 2H), 6.70-6.27 (m, 2H), 5.89 (s, 1H), 5.07 (s, 3H), 4.38- 4.20 (m, 2H), 3.94 (dd, J = 9.1, 3.8 Hz, 2H). | 502.1 |
| | | 4-(2,6-difluorobenzyl)-2-(4-((2-(3- | | | |
| | | (difluoromethoxy)azetidin-1-yl)pyridin- | | | |
| | | 4-yl)oxy)phenyl)-2,4-dihydro-3H- | | | |
| | | 1,2,4-triazol-3-one | | | |
| Example 288 Method 61 | +++ | 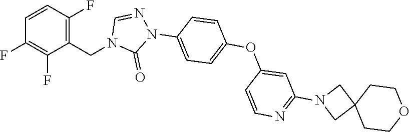 [SMILES: O=c1n(Cc2c(F)ccc(F)c2F)cnn1-c1ccc(Oc2ccnc(N3CC4(CCOCC4)C3)c2)cc1] | yellow solid, yield: 21% | ¹H NMR (400 MHz, CDCl₃) δ ppm 8.05 (s, 1H), 7.93 (m, 2H), 7.86 (d, J = 6.0 Hz, 1H), 7.34 (ddd, J = 18.9, 9.4, 5.1 Hz, 1H), 7.16 (m, 2H), 7.05 (m, 1H), 6.24 (dd, J = 6.0, 2.2 Hz, 1H), 5.78 (d, J = 2.2 Hz, 1H), 5.06 (s, 2H), 3.68 (s, 4H), 3.61 (m, 4H), 1.77 (m, 4H). | 524.0 |
| | | 2-(4-((2-(7-oxa-2-azaspiro[3.5]nonan- | | | |
| | | 2-yl)pyridin-4-yl)oxy)phenyl)-4- | | | |
| | | (2,3,6-trifluorobenzyl)-2,4-dihydro- | | | |
| | | 3H-1,2,4-triazol-3-one | | | |
| Example 289 Method 61 | ++++ | 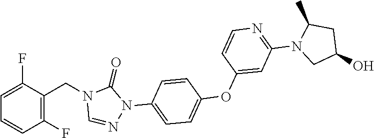 [SMILES: C[C@H]1C[C@@H](O)CN1c1cc(Oc2ccc(-n3ncn(Cc4c(F)cccc4F)c3=O)cc2)ccn1] | white solid, yield: 3.6% | ¹H NMR (400 MHz, CDCl₃) δ 8.07- 7.96 (t, 3H), 7.59 (s, 1H), 7.40- 7.32 (m, 1H), 7.16-7.10 (m, 2H), 6.98 (dd, J = 8.3, 7.5 Hz, 2H), 6.31- 6.19 (m, 1H), 5.88 (d, J = 2.1 Hz, 1H), 4.98 (s, 2H), 4.64-4.54 (m, 1H), 4.13 (s, 1H), 3.80-3.59 (m, 2H), 2.30 (m, 1H), 1.88-1.84 (m, 1H), 1.39 (d, J = 6.4 Hz, 3H). | 480.0 |
| | | 4-(2,6-difluorobenzyl)-2-(4-((2- | | | |
| | | ((2S,4R)-4-hydroxy-2-methylpyrrolidin- | | | |
| | | 1-yl)pyridin-4-yl)oxy)phenyl)-2,4- | | | |
| | | dihydro-3H-1,2,4-triazol-3-one | | | |
| Example 290 Method 61 | ++++ | 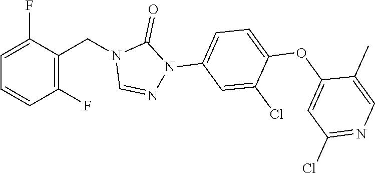 [SMILES: Cc1cnc(Cl)cc1Oc1ccc(-n2ncn(Cc3c(F)cccc3F)c2=O)cc1Cl] | off- white solid, yield: 50.0% | ¹H NMR (400 MHz, Chloroform-d) δ 8.24 (d, J = 2.4 Hz, 1H), 8.19 (d, J = 0.8 Hz, 1H), 8.04 (dd, J = 8.8, 2.4 Hz, 1H), 7.64-7.62 (m, 1H), 7.41-7.34 (m, 1H), 7.21 (d, J = 8.8 Hz, 1H), 7.05-6.88 (m, 2H), 6.38 (s, 1H), 5.00 (d, J = 1.2 Hz, 2H), 2.35 (s, 3H). | 463.2 |
| | | 2-(3-chloro-4-((2-chloro-5-methyl- | | | |
| | | pyridin-4-yl)oxy)phenyl)-4-(2,6- | | | |
| | | difluorobenzyl)-2,4-dihydro-3H-1,2,4- | | | |
| | | triazol-3-one | | | |
| Example 291 Method 61 | ++++ | 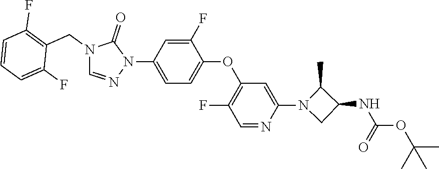 [SMILES: C[C@H]1[C@@H](NC(=O)OC(C)(C)C)CN1c1cc(Oc2ccc(-n3ncn(Cc4c(F)cccc4F)c3=O)cc2F)c(F)cn1] | yellow solid, yield: 80.3% | ¹H NMR (400 MHz, Chloroform-d) δ 8.03-7.95 (m, 2H), 7.88 (d, J = 9.2 Hz, 1H), 7.62 (s, 1H), 7.44- 7.32 (m, 1H), 7.22 (t, J = 8.8 Hz, 1H), 7.05-6.94 (m, 2H), 5.55 (d, J = 5.6 Hz, 1H), 5.02-4.97 (m, 2H), 4.97-4.91 (m, 1H), 4.61-4.38 (m, 2H), 3.76-3.71 (m, 1H), 1.44 (s, 9H), 1.29-1.23 (m, 3H) | 601.2 |
| | | tert-butyl | | | |
| | | ((2S,3S)-1-(4-(4-(4-(2,6-difluoro- | | | |
| | | benzyl)-5-oxo-4,5-dihydro-1H-1,2, | | | |
| | | 4-triazol-1-yl)-2-fluorophenoxy)- | | | |
| | | 5-fluoropyridin-2-yl)-2-methyl- | | | |
| | | azetidin-3-yl)carbamate | | | |
| Example 292 Method 61 | ++++ | 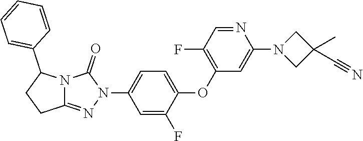 [SMILES: CC1(C#N)CN(c2cc(Oc3ccc(-n4nc5n(c4=O)C(c4ccccc4)CC5)cc3F)c(F)cn2)C1] | light yellow solid, yield: 7.1% | ¹H NMR (400 MHz, Chloroform-d) δ 8.03 (d, J = 3.2 Hz, 1H), 7.95 (dd, J = 12.0, 2.4 Hz, 1H), 7.82 (ddd, J = 9.2, 2.4, 1.6 Hz, 1H), 7.45- 7.33 (m, 3H), 7.27 (d, J = 1.6 Hz, 2H), 7.21 (t, J = 8.8 Hz, 1H), 5.56 (dd, J = 5.6, 1.2 Hz, 1H), 5.30 (dd, J = 7.6, 4.0 Hz, 1H), 4.25 (d, J = 7.6 Hz, 2H), 3.82 (d, J = 8.0 Hz, | 501.2 |
| | | 1-(5-fluoro-4-(2-fluoro-4-(3-oxo- | | 2H), 3.14-3.01 (m, 2H), 2.99- | |
| | | 5-phenyl-6,7-dihydro-3H-pyrrolo | | 2.90 (m, 1H), 2.61-2.49 (m, 1H), | |
| | | [2,1-c][1,2,4]triazol-2(5H)-yl)phenoxy) | | 1.71 (s, 3H). | |
| | | pyridin-2-yl)-3-methylazetidine- | | | |
| | | 3-carbonitrile | | | |
| Example 293 Method 61 | ++++ | 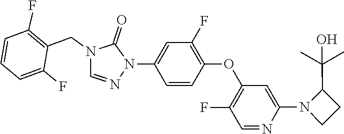 [SMILES: CC(C)(O)C1CCN1c1cc(Oc2ccc(-n3ncn(Cc4c(F)cccc4F)c3=O)cc2F)c(F)cn1] | White solid, yield: 14.4% | ¹H NMR (400 MHz, Chloroform-d) δ 7.97 (dd, J = 12.0, 2.4 Hz, 1H), 7.91 (d, J = 3.2 Hz, 1H), 7.86 (ddd, J = 9.2, 2.4, 1.6 Hz, 1H), 7.62 (s, 1H), 7.38 (tt, J = 8.4, 6.4 Hz, 1H), 7.25-7.19 (m, 2H), 7.05-6.94 (m, 2H), 5.53 (d, J = 5.6 Hz, 1H), 4.99 (d, J = 1.2 Hz, 2H), 4.29- 4.17 (m, 1H), 3.69-3.59 (m, 1H), | 530.2 |
| | | 4-(2,6-difluorobenzyl)-2-(3-fluoro- | | 3.56-3.46 (m, 1H), 2.24-2.12 | |
| | | 4-((5-fluoro-2-(2-(2-hydroxypropan- | | (m, 1H), 2.11-1.97 (m, 1H), 1.23 | |
| | | 2-yl)azetidin-1-yl)pyridin-4-yl) | | (s, 3H), 1.09 (s, 3H). | |
| | | oxy)phenyl)-2,4-dihydro-3H-1,2,4- | | | |
| | | triazol-3-one | | | |
| Example 294 Method 61 | ++++ | 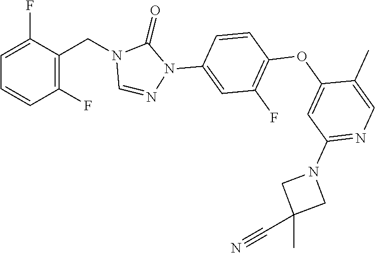 [SMILES: Cc1cnc(N2CC(C)(C#N)C2)cc1Oc1ccc(-n2ncn(Cc3c(F)cccc3F)c2=O)cc1F] | White solid, yield: 14.6% | ¹H NMR (400 MHz, Chloroform-d) δ 8.00-7.92 (m, 2H), 7.89-7.81 (m, 1H), 7.63 (d, J = 0.8 Hz, 1H), 7.38 (tt, J = 8.4, 6.4 Hz, 1H), 7.17 (t, J = 8.8 Hz, 1H), 7.05-6.94 (m, 2H), 5.42 (s, 1H), 4.99 (t, J = 1.2 Hz, 2H), 4.26 (d, J = 8.0 Hz, 2H), 3.85 (d, J = 8.0 Hz, 2H), 2.23 (s, 3H), 1.69 (s, 3H). | 507.2 |
| | | 1-(4-(4-(4-(2,6-difluorobenzyl)-5- | | | |
| | | oxo-4,5-dihydro-1H-1,2,4-triazol- | | | |
| | | 1-yl)-2-fluorophenoxy)-5-methyl- | | | |
| | | pyridin-2-yl)-3-methylazetidine-3- | | | |
| | | carbonitrile | | | |
| Example 295 Method 61 | ++++ | 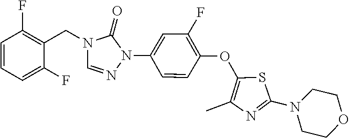 [SMILES: Cc1nc(N2CCOCC2)sc1Oc1ccc(-n2ncn(Cc3c(F)cccc3F)c2=O)cc1F] | white solid, yield: 41.7% | NA | 504.3 |
| | | 4-(2,6-difluorobenzyl)-2-(3-fluoro- | | | |
| | | 4-((4-methyl-2-morpholinothiazol- | | | |
| | | 5-yl)oxy)phenyl)-2,4-dihydro-3H- | | | |
| | | 1,2,4-triazol-3-one | | | |
| Example | ++++ | 4-(2,6-difluorobenzyl)-2-(3-fluoro- | white | NA | 517.2 |
| 296 | | 4-((4-methyl-2-(4-methylpiperazin- | solid, | | |
| Method | | 1-yl)thiazol-5-yl)oxy)phenyl)-2,4- | yield: | | |
| 61 | | dihydro-3H-1,2,4-triazol-3-one | 8.7% | | |
| Example 297 Method 61 | ++++ | 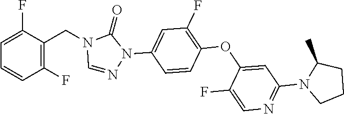 [SMILES: C[C@H]1CCCN1c1cc(Oc2ccc(-n3ncn(Cc4c(F)cccc4F)c3=O)cc2F)c(F)cn1] | white solid, yield: 9.02% | ¹H NMR (400 MHz, DMSO-d₆) δ 8.38 (s, 1H), 8.11 (d, J = 2.9 Hz, 1H), 7.92 (dd, J = 12.5, 2.6 Hz, 1H), 7.81-7.73 (m, 1H), 7.55-7.42 (m, 2H), 7.16 (t, J = 8.0 Hz, 2H), 5.75 (d, J = 5.6 Hz, 1H), 4.99 (s, 2H), 4.00-3.93 (m, 1H), 3.29-3.23 (m, 1H), 3.06 (q, J = 8.8 Hz, 1H), | 500.2 |
| | | (S)-4-(2,6-difluorobenzyl)-2-(3- | | 2.06-1.70 (m, 4H), 1.04 (d, J = 6.2 | |
| | | fluoro-4-((5-fluoro-2-(2-methyl- | | Hz, 3H). | |
| | | pyrrolidin-1-yl)pyridin-4-yl)oxy) | | | |
| | | phenyl)-2,4-dihydro-3H-1,2,4-triazol- | | | |
| | | 3-one | | | |
| Example | ++++ | 2-(3-chloro-4-((2-((4,4-dimethyl- | white | ¹H NMR (400 MHz, DMSO-d₆) δ | 547.1 |
| 298 | | 4,5-dihydrooxazol-2-yl)amino)-4- | solid, | 8.45 (s, 1H), 8.33 (s, 1H), 8.05 (d, | |
| Method | | methylthiazol-5-yl)oxy)phenyl)-4- | yield: | J = 2.6 Hz, 1H), 7.78 (dd, J = 9.1, | |
| 61 | | (2,6-difluorobenzyl)-2,4-dihydro- | 18.97% | 2.6 Hz, 1H), 7.52-7.44 (m, 1H), | |
| | | 3H-1,2,4-triazol-3-one | | 7.19-7.12 (m, 3H), 7.05 (s, 1H), | |
| | | | | 4.97 (s, 2H), 4.14 (s, 2H), 2.10 (s, | |
| | | | | 3H), 1.65 (s, 6H). | |
| Example 299 Method 61 | ++++ | 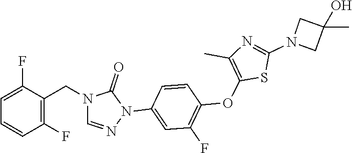 [SMILES: Cc1nc(N2CC(C)(O)C2)sc1Oc1ccc(-n2ncn(Cc3c(F)cccc3F)c2=O)cc1F] | yellow solid, yield: 23.13% | ¹H NMR (400 MHz, DMSO-d₆) δ 8.34 (s, 1H), 7.86 (dd, J = 12.7, 2.5 Hz, 1H), 7.69-7.65 (m, 1H), 7.43-7.35 (m, 1H), 7.30-7.09 (m, 3H), 5.71 (s, 1H), 4.95 (s, 2H), 3.80 (d, J = 1.9 Hz, 4H), 2.02 (s, 3H), 1.42 (s, 3H). | 504.2 |
| | | 4-(2,6-difluorobenzyl)-2-(3-fluoro- | | | |
| | | 4-((2-(3-hydroxy-3-methylazetidin- | | | |
| | | 1-yl)-4-methylthiazol-5-yl)oxy) | | | |
| | | phenyl)-2,4-dihydro-3H-1,2,4- | | | |
| | | triazol-3-one | | | |
| Example 300 Method 61 | +++ | 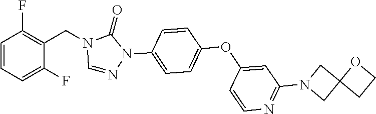 [SMILES: O=c1n(Cc2c(F)cccc2F)cnn1-c1ccc(Oc2ccnc(N3CC4(CCO4)C3)c2)cc1] | white solid, yield: 46.70% | ¹H NMR (400 MHz, DMSO-d₆) δ 8.36 (s, 1H), 8.04-7.94 (m, 3H), 7.52-7.44 (m, 1H), 7.36-7.27 (m, 2H), 7.22-7.11 (m, 2H), 6.53 (dd, J = 7.0, 2.4 Hz, 1H), 6.05 (d, J = 2.4 Hz, 1H), 5.00 (s, 2H), 4.45-4.36 (m, 4H), 4.24 (dd, J = 10.5, 1.6 Hz, 2H), 2.86 (t, J = 7.5 Hz, 2H). | 478.2 |
| | | 4-[(2,6-difluorophenyl)methyl]-2- | | | |
| | | [4-[[2-(1-oxa-6-azaspiro[3.3]heptan- | | | |
| | | 6-yl)-4-pyridyl]oxy]phenyl]-1,2,4- | | | |
| | | triazol-3-one | | | |
| Example 301 Method 61 | ++++ | 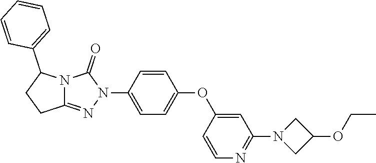 [SMILES: CCOC1CN(c2cc(Oc3ccc(-n4nc5n(c4=O)C(c4ccccc4)CC5)cc3)ccn2)C1] | white solid, yield: 26.35% | ¹H NMR (400 MHz, DMSO-d₆) δ 8.02-7.90 (m, 3H), 7.45-7.25 (m, 7H), 6.50 (d, J = 6.9 Hz, 1H), 6.02 (d, J = 2.3 Hz, 1H), 5.33 (dd, J = 7.9, 4.7 Hz, 1H), 4.42-4.39 (m, 1H), 4.32-4.28 (m, 2H), 3.97-3.91 (m, 2H), 3.46-3.41 (q, J = 7.0 Hz, 2H), 3.06-2.99 (m, 2H), 2.97-2.89 (m, 1H), 2.37-2.30 (m, 1H), 1.13 (t, J = 7.0 Hz, 3H). | 470.2 |
| | | 2-[4-[[2-(3-ethoxyazetidin-1-yl)-4- | | | |
| | | pyridyl]oxy]phenyl]-5-phenyl-6,7- | | | |
| | | dihydro-5H-pyrrolo[2,1-c][1,2,4] | | | |
| | | triazol-3-one | | | |
| Example 302 Method 61 | ++++ | 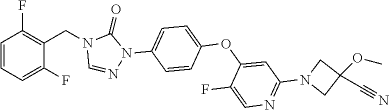 [SMILES: COC1(C#N)CN(c2cc(Oc3ccc(-n4ncn(Cc5c(F)cccc5F)c4=O)cc3)c(F)cn2)C1] | yellow solid, yield: 4.24% | ¹H NMR (400 MHz, DMSO-d₆) δ 8.33 (s, 1H), 8.18 (d, J = 3.0 Hz, 1H), 7.96-7.88 (m, 2H), 7.53-7.45 (m, 1H), 7.33-7.25 (m, 2H), 7.22-7.10 (m, 2H), 5.98 (d, J = 5.6 Hz, 1H), 4.99 (s, 2H), 4.35-4.24 (m, 2H), 4.05-3.96 (m, 2H), 3.37 | 509.2 |
| | | 1-(4-(4-(4-(2,6-difluorobenzyl)-5- | | (s, 3H). | |
| | | oxo-4,5-dihydro-1H-1,2,4-triazol- | | | |
| | | 1-yl)phenoxy)-5-fluoropyridin-2- | | | |
| | | yl)-3-methoxyazetidine-3-carbonitrile | | | |
| Example 303 Method 61 | ++++ | 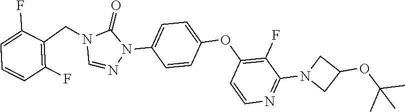 [SMILES: CC(C)(C)OC1CN(c2nccc(Oc3ccc(-n4ncn(Cc5c(F)cccc5F)c4=O)cc3)c2F)C1] | white solid, yield: 5.70% | ¹H NMR (400 MHz, DMSO-d₆) δ 8.31 (s, 1H), 7.95-7.86 (m, 2H), 7.78 (d, J = 5.6 Hz, 1H), 7.54-7.41 (m, 1H), 7.27-7.20 (m, 2H), 7.16 (t, J = 8.1 Hz, 2H), 6.27 (t, J = 5.5 Hz, 1H), 4.98 (s, 2H), 4.68-4.62 (m, 1H), 4.30-4.25 (m, 2H), 3.87-3.78 (m, 2H), 1.15 (s, 9H). | 526.2 |
| | | 2-(4-((2-(3-(tert-butoxy)azetidin-1- | | | |
| | | yl)-3-fluoropyridin-4-yl)oxy)phenyl)- | | | |
| | | 4-(2,6-difluorobenzyl)-2,4-dihydro- | | | |
| | | 3H-1,2,4-triazol-3-one | | | |
| Example 304 Method 61 | +++ | 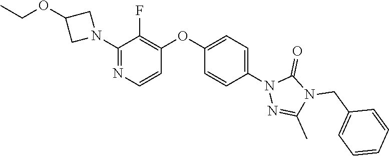 [SMILES: CCOC1CN(c2nccc(Oc3ccc(-n4nc(C)n(Cc5ccccc5)c4=O)cc3)c2F)C1] | yellow solid, yield: 3.37% | ¹H NMR (400 MHz, DMSO-d₆) δ 8.00-7.90 (m, 2H), 7.79 (d, J = 5.6 Hz, 1H), 7.44-7.20 (m, 7H), 6.28 (t, J = 5.5 Hz, 1H), 4.92 (s, 2H), 4.44-4.38 (m, 1H), 4.30-4.25 (m, 2H), 3.94-3.82 (m, 2H), 3.45 (q, J = 7.0 Hz, 2H), 2.22 (s, 3H), 1.14 (t, J = 7.0 Hz, 3H). | 476.2 |
| | | 4-benzyl-2-[4-[[2-(3-ethoxyazetidin- | | | |
| | | 1-yl)-3-fluoro-4-pyridyl]oxy]phenyl]- | | | |
| | | 5-methyl-1,2,4-triazol-3-one | | | |
| Example 305 Method 61 | +++ | 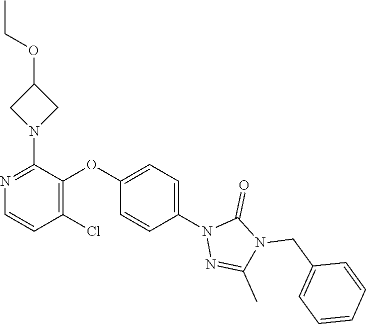 [SMILES: CCOC1CN(c2nccc(Cl)c2Oc2ccc(-n3nc(C)n(Cc4ccccc4)c3=O)cc2)C1] | yellow solid, yield: 15.71% | ¹H NMR (400 MHz, DMSO-d₆) δ 7.99 (d, J = 5.3 Hz, 1H), 7.87-7.75 (m, 2H), 7.42-7.25 (m, 5H), 6.95-6.83 (m, 3H), 4.91 (s, 2H), 4.34-4.09 (m, 3H), 3.78 (dd, J = 9.6, 4.3 Hz, 2H), 3.36 (t, J = 7.0 Hz, 2H), 2.20 (s, 3H), 1.07 (t, J = 7.0 Hz, 3H). | 492.2 |
| | | 4-benzyl-2-[4-[[4-chloro-2-(3-ethoxy- | | | |
| | | azetidin-1-yl)-3-pyridyl]oxy]phenyl]- | | | |
| | | 5-methyl-1,2,4-triazol-3-one | | | |
| Example 306 Method 61 | +++ | 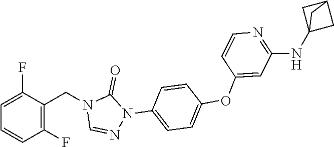 [SMILES: O=c1n(Cc2c(F)cccc2F)cnn1-c1ccc(Oc2ccnc(NC34CC(C3)C4)c2)cc1] | white solid, yield: 2.84% | ¹H NMR (400 MHz, DMSO-d₆) δ 8.35 (s, 1H), 8.02-7.87 (m, 3H), 7.53-7.45 (m, 1H), 7.33 (d, J = 8.6 Hz, 2H), 7.23-7.11 (m, 2H), 6.52 (s, 1H), 6.07 (s, 1H), 4.99 (s, 2H), 2.53 (s, 2H), 2.04 (s, 6H). | 462.2 |
| | | 2-[4-[[2-(1-bicyclo[1.1.1]pentanyl- | | | |
| | | amino)-4-pyridyl]oxy]phenyl]-4- | | | |
| | | [(2,6-difluorophenyl)methyl]-1,2,4- | | | |
| | | triazol-3-one | | | |
| Example | +++ | 2-(4-((2-chloro-4-(7-oxa-2-azaspiro | | ¹NMR (400 MHz, DMSO-d₆) δ | 540.0 |
| 307 | | [3.5]nonan-2-yl)pyridin-3-yl)oxy) | | 7.96 (d, J = 5.9 Hz, 1H), 7.91- | |
| Method | | phenyl)-4-(2,6-difluorobenzyl)- | | 7.82 (m, 1H), 7.79-7.71 (m, 2H), | |
| 61 | | 2,4-dihydro-3H-1,2,4-triazol-3-one | | 7.48-7.36 (m, 1H), 7.09-6.97 | |
| | | | | (m, 2H), 6.88-6.73 (m, 3H), 5.01 | |
| | | | | (s, 2H), 3.81 (s, 4H), 3.57-3.51 | |
| | | | | (m, 4H), 1.65 (t, J = 13.4 Hz, 4H). | |
| Example | +++ | 3-((4-(4-(4-(2,6-difluorobenzyl)-5- | white | ¹H NMR (400 MHz, MeOD) δ 8.10- | 432.2 |
| 308 | | oxo-4,5-dihydro-1H-1,2,4-triazol- | solid, | 8.02 (m, 2H), 7.97 (d, J = 2.4 Hz, | |
| Method | | 1-yl)-2-methylphenoxy)-5-fluoro- | yield: | 1H), 7.88 (dd, J = 8.8, 2.5 Hz, 1H), | |
| 61 | | pyridin-2-yl)amino)bicyclo[1.1.1] | 5.1% | 7.48 (ddd, J = 13.1, 8.5, 6.6 Hz, | |
| | | pentane-1-carbonitrile | | 1H), 7.22 (d, J = 8.8 Hz, 1H), 7.09 | |
| | | | | (t, J = 8.1 Hz, 2H), 5.84 (d, J = 6.3 | |
| | | | | Hz, 1H), 5.08 (s, 2H), 2.51 (s, 6H), | |
| | | | | 2.25 (s, 3H). | |
| Example 309 Method 61 | ++++ | 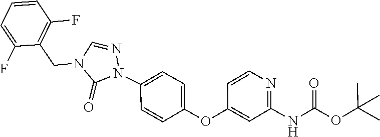 [SMILES: CC(C)(C)OC(=O)Nc1cc(Oc2ccc(-n3ncn(Cc4c(F)cccc4F)c3=O)cc2)ccn1] | yellow solid, yield: 9.27% | ¹H NMR (400 MHz, DMSO-d₆) δ 9.82 (s, 1H), 8.33 (s, 1H), 8.12 (d, J = 5.7 Hz, 1H), 8.00-7.91 (m, 2H), 7.54-7.44 (m, 1H), 7.34 (d, J = 2.3 Hz, 1H), 7.31-7.23 (m, 2H), 7.17 (t, J = 8.1 Hz, 2H), 6.61 (dd, J = 5.8, 2.4 Hz, 1H), 4.99 (s, 2H), 1.40 (s, 9H). | 496.2 |
| | | tert-butyl | | | |
| | | (4-(4-(4-(2,6-difluorobenzyl)-5-oxo- | | | |
| | | 4,5-dihydro-1H-1,2,4-triazol-1- | | | |
| | | yl)phenoxy)pyridin-2-yl)carbamate | | | |
| Example | +++ | 2-(4-((2-aminopyridin-4-yl)oxy) | white | ¹H NMR (400 MHz, DMSO-d₆) δ | 396.1 |
| 310 | | phenyl)-4-(2,6-difluorobenzyl)-2,4- | solid, | 13.11 (s, 1H), 8.36 (s, 1H), | |
| Method | | dihydro-3H-1,2,4-triazol-3-one | yield: | 8.11-7.98 (m, 2H), 7.94 (d, J = 7.2 | |
| 61 | | | 32.3% | Hz, 1H), 7.67 (brs, 2H), 7.53-7.44 | |
| | | | | (m, 1H), 7.41-7.27 (m, 2H), 7.17 (t, | |
| | | | | J = 8.1 Hz, 2H), 6.66 (dd, J = 7.2, | |
| | | | | 2.5 Hz, 1H), 6.07 (d, J = 2.5 Hz, | |
| | | | | 1H), 5.00 (s, 2H). | |
| Example 311 Method 61 | +++ | 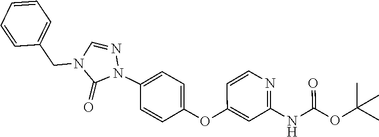 [SMILES: CC(C)(C)OC(=O)Nc1cc(Oc2ccc(-n3ncn(Cc4ccccc4)c3=O)cc2)ccn1] | white solid, yield: 1.18% | ¹H NMR (400 MHz, DMSO-d₆) δ 9.82 (s, 1H), 8.40 (s, 1H), 8.13 (d, J = 5.4 Hz, 1H), 7.99 (d, J = 8.6 Hz, 2H), 7.44-7.25 (m, 8H), 6.62 (d, J = 5.1 Hz, 1H), 4.91 (s, 2H), 1.41 (s, 9H). | 460.2 |
| | | tert-butyl | | | |
| | | (4-(4-(4-benzyl-5-oxo-4,5-dihydro- | | | |
| | | 1H-1,2,4-triazol-1-yl)phenoxy) | | | |
| | | pyridin-2-yl)carbamate | | | |
| Example | ++++ | 2-(4-((2-(azetidin-3-ylmethyl)-4- | White | ¹H NMR (400 MHz, Chloroform-d) | 470.2 |
| 312 | | methylthiazol-5-yl)oxy)phenyl)-4- | solid, | δ 7.89 (d, J = 8.8 Hz, 2H), 7.56 (s, | |
| Method | | (2,6-difluorobenzyl)-2,4-dihydro- | yield: | 1H), 7.36 (p, J = 7.6 Hz, 1H), 7.04- | |
| 61 | | 3H-1,2,4-triazol-3-one | 22.8% | 6.93 (m, 4H), 4.97 (s, 2H), 4.23 | |
| | | | | (t, J = 9.6 Hz, 2H), 3.93 (d, J = 9.6 | |
| | | | | Hz, 2H), 3.39 (d, J = 7.6 Hz, 1H), | |
| | | | | 3.24 (d, J = 7.6 Hz, 2H), 2.18 (s, | |
| | | | | 3H). | |
| Example | +++ | 3-((3-chloro-4-(2-chloro-4-(4-(2,6- | light | ¹H NMR (400 MHz, Chloroform-d) | 555.2 |
| 313 | | difluorobenzyl)-5-oxo-4,5-dihydro- | yellow | δ 8.19 (d, J = 2.4 Hz, 1H), 7.96 | |
| Method | | 1H-1,2,4-triazol-1-yl)phenoxy) | solid, | (dd, J = 8.8, 2.4 Hz, 1H), 7.84 (d, | |
| 61 | | pyridin-2-yl)amino)bicyclo[1.1.1] | yield: | J = 5.6 Hz, 1H), 7.61 (s, 1H), 7.38 | |
| | | pentane-1-carbonitrile | 19.3% | (tt, J = 8.4, 6.4 Hz, 1H), 7.15 (d, J = | |
| | | | | 9.2 Hz, 1H), 7.05-6.94 (m, 2H), | |
| | | | | 5.94 (d, J = 5.7 Hz, 1H), 5.56 (brs, | |
| | | | | 1H), 4.98 (d, J = 1.2 Hz, 2H), 2.67 | |
| | | | | (s, 6H). | |
| Example | ++++ | 1-(4-(2-chloro-4-(4-(2,6-difluoro- | white | ¹H NMR (400 MHz, Chloroform-d) | 525.2 |
| 314 | | benzyl)-5-oxo-4,5-dihydro-1H-1,2,4- | solid, | δ 8.20 (d, J = 2.4 Hz, 1H), 8.03 (d, | |
| Method | | triazol-1-yl)phenoxy)pyridin-2- | yield: | J = 6.0 Hz, 1H), 7.98 (dd, J = 8.8, | |
| 61 | | yl)-3-methoxyazetidine-3-carbonitrile | 4.2% | 2.4 Hz, 1H), 7.63 (s, 1H), 7.38 (tt, | |
| | | | | J = 8.4, 6.4 Hz, 1H), 7.18 (d, J = 8.8 | |
| | | | | Hz, 1H), 7.04-6.96 (m, 2H), 6.31 | |
| | | | | (dd, J = 6.0, 2.0 Hz, 1H), 5.71 (d, | |
| | | | | J = 2.0 Hz, 1H), 5.00 (d, J = 1.2 Hz, | |
| | | | | 2H), 4.48-4.37 (m, 2H), 4.15 (d, | |
| | | | | J = 9.2 Hz, 2H), 3.47 (s, 3H). | |
| Example 315 Method 61 | ++++ | 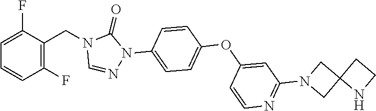 [SMILES: O=c1n(Cc2c(F)cccc2F)cnn1-c1ccc(Oc2ccnc(N3CC4(CCN4)C3)c2)cc1] | white solid, yield: 34.5% | ¹H NMR (400 MHz, DMSO-d₆) δ 8.32 (s, 1H), 7.95-7.89 (m, 3H), 7.66-7.42 (m, 1H), 7.23-7.18 (m, 3H), 7.17-7.14 (m, 1H), 6.19 (dd, J = 5.6, 2.0 Hz, 1H), 5.79 (d, J = 2.0 Hz, 1H), 4.99 (s, 2H), 3.99 (d, J = 8.8 Hz, 2H), 3.82 (d, J = 8.8 Hz, 2H), 3.26 (t, J = 7.2 Hz, 2H), 2.40 (t, J = 7.2 Hz, 2H). | 477.2 |
| | | 2-(4-((2-(1,6-diazaspiro[3.3]heptan- | | | |
| | | 6-yl)pyridin-4-yl)oxy)phenyl)-4- | | | |
| | | (2,6-difluorobenzyl)-2,4-dihydro- | | | |
| | | 3H-1,2,4-triazol-3-one | | | |
| Example 316 Method 61 | ++++ | 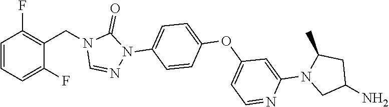 [SMILES: C[C@H]1CC(N)CN1c1cc(Oc2ccc(-n3ncn(Cc4c(F)cccc4F)c3=O)cc2)ccn1] | white solid, yield: 7.5% | ¹H NMR (400 MHz, DMSO-d₆) δ 8.32 (s, 1H), 7.95 (d, J = 5.6 Hz, 1H), 7.93-7.87 (m, 2H), 7.53- 7.43 (m, 1H), 7.26-7.11 (m, 4H), 6.12 (dd, J = 5.6, 2.0 Hz, 1H), 5.79 (d, J = 2.0 Hz, 1H), 4.98 (s, 2H), 4.07 (q, J = 6.0 Hz, 1H), 3.60 (q, | 479.2 |
| | | 2-(4-((2-((2S,4S)-4-amino-2-methyl- | | J = 7.2 Hz, 1H), 3.47 (dd, J = 10.0, | |
| | | pyrrolidin-1-yl)pyridin-4-yl)oxy) | | 6.8 Hz, 1H), 2.82 (dd, J = 10.0, 7.2 | |
| | | phenyl)-4-(2,6-difluorobenzyl)-2,4- | | Hz, 1H), 2.67 (p, J = 1.6 Hz, 1H), | |
| | | dihydro-3H-1,2,4-triazol-3-one | | 2.33 (p, J = 2.0 Hz, 1H), 1.80- | |
| | | | | 1.72 (m, 2H), 1.11 (d, J = 6.4 Hz, | |
| | | | | 3H). | |
| Example 317 Method 61 | ++ | 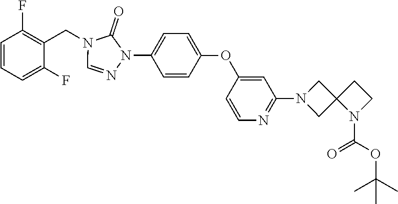 [SMILES: CC(C)(C)OC(=O)N1CCC12CN(c1cc(Oc3ccc(-n4ncn(Cc5c(F)cccc5F)c4=O)cc3)ccn1)C2] | light yellow solid, yield: 45.5% | ¹H NMR (400 MHz, Chloroform-d) δ 8.03-7.94 (m, 3H), 7.59 (s, 1H), 7.43-7.31 (m, 1H), 7.10 (d, J = 8.8 Hz, 2H), 7.04-6.94 (m, 2H), 6.22 (dd, J = 6.0, 2.0 Hz, 1H), 5.74 (d, J = 2.0 Hz, 1H), 4.99 (d, J = 1.6 Hz, 2H), 4.42 (d, J = 8.8 Hz, 2H), 4.03 (d, J = 8.8 Hz, 2H), 3.81 (t, J = 7.2 Hz, 2H), 2.45 (t, J = 7.2 Hz, 2H), 1.29 (s, 9H). | 577.2 |
| | | tert-butyl | | | |
| | | 6-(4-(4-(4-(2,6-difluorobenzyl)-5- | | | |
| | | oxo-4,5-dihydro-1H-1,2,4-triazol- | | | |
| | | 1-yl)phenoxy)pyridin-2-yl)-1,6-di- | | | |
| | | azaspiro[3.3]heptane-1-carboxylate | | | |
| Example 318 Method 61 | +++ | 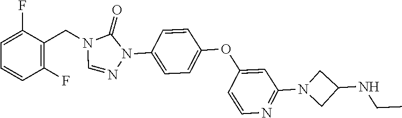 [SMILES: CCNC1CN(c2cc(Oc3ccc(-n4ncn(Cc5c(F)cccc5F)c4=O)cc3)ccn2)C1] | white solid, yield: 21.5% | ¹H NMR (400 MHz, Chloroform-d) δ 8.03-7.93 (m, 3H), 7.59 (s, 1H), 7.43-7.31 (m, 1H), 7.15-7.07 (m, 2H), 7.04-6.93 (m, 2H), 6.22 (dd, J = 6.0, 2.0 Hz, 1H), 5.73 (d, J = 2.0 Hz, 1H), 4.98 (d, J = 1.6 Hz, 2H), 4.18 (dd, J = 8.0, 6.8 Hz, 2H), 3.83-3.65 (m, 3H), 2.68 (q, J = | 479.2 |
| | | 4-(2,6-difluorobenzyl)-2-(4-((2-(3- | | 7.2 Hz, 2H), 1.15 (t, J = 7.2 Hz, | |
| | | (ethylamino)azetidin-1-yl)pyridin- | | 3H). | |
| | | 4-yl)oxy)phenyl)-2,4-dihydro-3H- | | | |
| | | 1,2,4-triazol-3-one | | | |
| Example 319 Method 61 | ++++ | 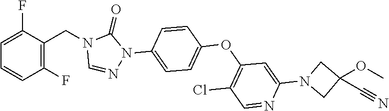 [SMILES: COC1(C#N)CN(c2cc(Oc3ccc(-n4ncn(Cc5c(F)cccc5F)c4=O)cc3)c(Cl)cn2)C1] | white solid, yield: 10.8% | ¹H NMR (400 MHz, DMSO-d₆) δ 8.33 (s, 1H), 8.19 (s, 1H), 7.99- 7.90 (m, 2H), 7.48 (tt, J = 8.4, 6.4 Hz, 1H), 7.31-7.23 (m, 2H), 7.22- 7.11 (m, 2H), 5.84 (s, 1H), 4.99 (s, 2H), 4.30 (dd, J = 9.6, 1.2 Hz, 2H), 4.01 (dd, J = 9.6, 1.2 Hz, 2H), | 525.2 |
| | | 1-(5-chloro-4-(4-(4-(2,6-difluoro- | | 3.37 (s, 3H). | |
| | | benzyl)-5-oxo-4,5-dihydro-1H-1,2,4- | | | |
| | | triazol-1-yl)phenoxy)pyridin-2- | | | |
| | | yl)-3-methoxyazetidine-3-carbonitrile | | | |
| Example 320 Method 61 | ++++ | 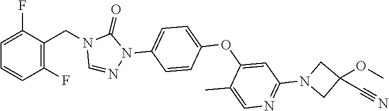 [SMILES: COC1(C#N)CN(c2cc(Oc3ccc(-n4ncn(Cc5c(F)cccc5F)c4=O)cc3)c(C)cn2)C1] | off- white solid, yield: 23.5% | ¹H NMR (400 MHz, DMSO-d₆) δ 8.32 (s, 1H), 7.97 (s, 1H), 7.95- 7.86 (m, 2H), 7.49 (tt, J = 8.4, 6.4 Hz, 1H), 7.23-7.09 (m, 4H), 5.74 (s, 1H), 4.99 (s, 2H), 4.28-4.21 (m, 2H), 3.94 (d, J = 9.6 Hz, 2H), 3.36 (s, 3H), 2.11 (s, 3H). | 505.2 |
| | | 1-(4-(4-(4-(2,6-difluorobenzyl)-5- | | | |
| | | oxo-4,5-dihydro-1H-1,2,4-triazol- | | | |
| | | 1-yl)phenoxy)-5-methylpyridin-2- | | | |
| | | yl)-3-methoxyazetidine-3-carbonitrile | | | |
| Example 321 Method 61 | ++++ | 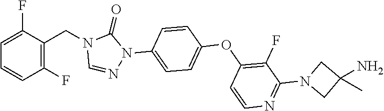 [SMILES: CC1(N)CN(c2nccc(Oc3ccc(-n4ncn(Cc5c(F)cccc5F)c4=O)cc3)c2F)C1] | Off- white solid, yield: 7.5% | ¹H NMR (400 MHz, DMSO-d₆) δ 8.38-8.29 (m, 4H), 7.96-7.89 (m, 2H), 7.87-7.81 (m, 1H), 7.49 (t, J = 8.4 Hz, 1H), 7.26-7.13 (m, 4H), 6.36 (t, J = 5.6 Hz, 1H), 4.98 (s, 2H), 4.16 (d, J = 8.8 Hz, 2H), 4.05 (d, J = 8.8 Hz, 2H), 1.58 (s, | 483.2 |
| | | 2-(4-((2-(3-amino-3-methylazetidin- | | 3H). | |
| | | 1-yl)-3-fluoropyridin-4-yl)oxy) | | | |
| | | phenyl)-4-(2,6-difluorobenzyl)-2,4- | | | |
| | | dihydro-3H-1,2,4-triazol-3-one | | | |
| Example 322 Method 61 | +++ | 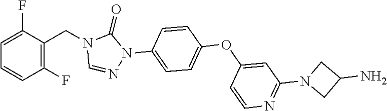 [SMILES: NC1CN(c2cc(Oc3ccc(-n4ncn(Cc5c(F)cccc5F)c4=O)cc3)ccn2)C1] | white semi- solid, yield: 9.1% | ¹H NMR (400 MHz, DMSO-d₆) δ 8.38-8.25 (m, 3H), 8.02 (dd, J = 6.4, 1.2 Hz, 1H), 7.96 (dd, J = 8.8, 1.6 Hz, 2H), 7.48 (tt, J = 8.4, 6.4 Hz, 1H), 7.28-7.21 (m, 2H), 7.16 (t, J = 8.0 Hz, 2H), 6.40 (s, 1H), 6.11 (s, 1H), 4.99 (s, 2H), 4.31- 4.22 (m, 2H), 4.17-4.07 (m, 1H), | 451.2 |
| | | 2-(4-((2-(3-aminoazetidin-1-yl)pyridin- | | 4.03-3.92 (m, 2H). | |
| | | 4-yl)oxy)phenyl)-4-(2,6-difluoro- | | | |
| | | benzyl)-2,4-dihydro-3H-1,2,4- | | | |
| | | triazol-3-one | | | |
| Example 323 Method 61 | +++ | 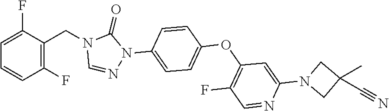 [SMILES: CC1(C#N)CN(c2cc(Oc3ccc(-n4ncn(Cc5c(F)cccc5F)c4=O)cc3)c(F)cn2)C1] | light brown solid, yield: 18.9% | ¹H NMR (400 MHz, Chloroform-d) δ 8.07-7.99 (m, 3H), 7.60 (s, 1H), 7.43-7.31 (m, 1H), 7.19-7.10 (m, 2H), 7.05-6.93 (m, 2H), 5.62 (d, J = 5.6 Hz, 1H), 4.99 (d, J = 1.2 Hz, 2H), 4.22 (d, J = 7.6 Hz, 2H), 3.79 (d, J = 7.6 Hz, 2H), 1.70 (s, | 493.2 |
| | | 1-(4-(4-(4-(2,6-difluorobenzyl)-5- | | 3H). | |
| | | oxo-4,5-dihydro-1H-1,2,4-triazol- | | | |
| | | 1-yl)phenoxy)-5-fluoropyridin-2- | | | |
| | | yl)-3-methylazetidine-3-carbonitrile | | | |
| Example 324 Method 61 | ++++ | 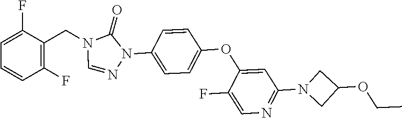 [SMILES: CCOC1CN(c2cc(Oc3ccc(-n4ncn(Cc5c(F)cccc5F)c4=O)cc3)c(F)cn2)C1] | light brown solid, yield: 37.5% | ¹H NMR (400 MHz, Chloroform-d) δ 8.04-7.95 (m, 3H), 7.59 (s, 1H), 7.42-7.30 (m, 1H), 7.18-7.09 (m, 2H), 7.04-6.93 (m, 2H), 5.64 (d, J = 5.6 Hz, 1H), 4.98 (s, 2H), 4.34 (tt, J = 6.4, 4.4 Hz, 1H), 4.07 (dd, J = 8.4, 6.4 Hz, 2H), 3.74 (dd, J = 8.4, 4.4 Hz, 2H), 3.44 (q, J = | 498.2 |
| | | 4-(2,6-difluorobenzyl)-2-(4-((2-(3- | | 7.2 Hz, 2H), 1.20 (t, J = 7.2 Hz, | |
| | | ethoxyazetidin-1-yl)-5-fluoropyridin- | | 3H). | |
| | | 4-yl)oxy)phenyl)-2,4-dihydro- | | | |
| | | 3H-1,2,4-triazol-3-one | | | |
| Example 325 Method 61 | ++++ | 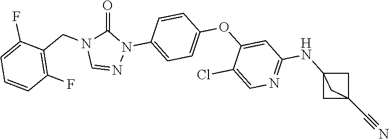 [SMILES: N#CC12CC(Nc3cc(Oc4ccc(-n5ncn(Cc6c(F)cccc6F)c5=O)cc4)c(Cl)cn3)(C1)C2] | white solid, yield: 3.4% | ¹H NMR (400 MHz, Chloroform-d) δ 8.19-8.12 (m, 2H), 7.98 (s, 1H), 7.64 (s, 1H), 7.44-7.32 (m, 1H), 7.20-7.14 (m, 2H), 6.99 (dd, J = 8.4, 7.2 Hz, 2H), 5.81 (s, 1H), 5.00 (d, J = 1.2 Hz, 2H), 2.41 (s, 6H). | 521.2 |
| | | 3-((5-chloro-4-(4-(4-(2,6-difluoro- | | | |
| | | benzyl)-5-oxo-4,5-dihydro-1H-1,2,4- | | | |
| | | triazol-1-yl)phenoxy)pyridin-2- | | | |
| | | yl)amino)bicyclo[1.1.1]pentane-1- | | | |
| | | carbonitrile | | | |
| Example 326 Method 61 | ++++ | 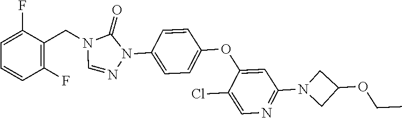 [SMILES: CCOC1CN(c2cc(Oc3ccc(-n4ncn(Cc5c(F)cccc5F)c4=O)cc3)c(Cl)cn2)C1] | Off- white solid, yield: 8.6% | ¹H NMR (400 MHz, Chloroform-d) δ 8.09 (s, 1H), 8.07-7.98 (m, 2H), 7.60 (d, J = 0.8 Hz, 1H), 7.43- 7.31 (m, 1H), 7.18-7.10 (m, 2H), 7.02-6.94 (m, 2H), 5.53 (s, 1H), 4.99 (d, J = 1.2 Hz, 2H), 4.34 (tt, J = 6.4, 4.4 Hz, 1H), 4.15-4.07 (m, 2H), 3.78 (dd, J = 9.2, 4.4 Hz, 2H), | 514.2 |
| | | 2-(4-((5-chloro-2-(3-ethoxyazetidin- | | 3.43 (q, J = 7.2 Hz, 2H), 1.19 (t, J = | |
| | | 1-yl)pyridin-4-yl)oxy)phenyl)-4- | | 7.2 Hz, 3H). | |
| | | (2,6-difluorobenzyl)-2,4-dihydro- | | | |
| | | 3H-1,2,4-triazol-3-one | | | |
| Example 327 Method 61 | ++++ | 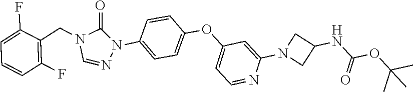 [SMILES: CC(C)(C)OC(=O)NC1CN(c2cc(Oc3ccc(-n4ncn(Cc5c(F)cccc5F)c4=O)cc3)ccn2)C1] | Off- white solid, yield: 10.0% | ¹H NMR (400 MHz, Chloroform-d) δ 8.03-7.93 (m, 3H), 7.60 (d, J = 0.8 Hz, 1H), 7.37 (tt, J = 8.4, 6.4 Hz, 1H), 7.15-7.07 (m, 2H), 7.04- 6.93 (m, 2H), 6.24 (dd, J = 6.0, 2.4 Hz, 1H), 5.74 (d, J = 2.0 Hz, | 551.2 |
| | | tert-butyl | | 1H), 4.99 (d, J = 1.2 Hz, 2H), 4.64- | |
| | | (1-(4-(4-(4-(2,6-difluorobenzyl)-5- | | 4.53 (m, 1H), 4.28 (t, J = 8.0 Hz, | |
| | | oxo-4,5-dihydro-1H-1,2,4-triazol- | | 2H), 3.74 (dd, J = 8.4, 5.2 Hz, 2H), | |
| | | 1-yl)phenoxy)pyridin-2-yl)azetidin- | | 1.44 (s, 9H). | |
| | | 3-yl)carbamate | | | |
| Example 328 Method 61 | +++ | 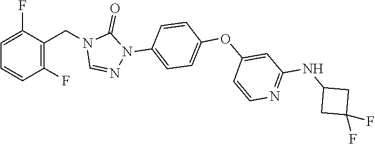 [SMILES: O=c1n(Cc2c(F)cccc2F)cnn1-c1ccc(Oc2ccnc(NC3CC(F)(F)C3)c2)cc1] | Off- white solid, yield: 27.1% | ¹H NMR (400 MHz, DMSO-d₆) δ 8.32 (brs, 1H), 7.96-7.87 (m, 3H), 7.55-7.43 (m, 1H), 7.29-7.19 (m, 2H), 7.17 (t, J = 8.0 Hz, 2H), 7.04 (d, J = 6.0 Hz, 1H), 6.23 (dd, J = 5.6, 2.0 Hz, 1H), 5.84 (d, J = 2.0 Hz, 1H), 4.99 (s, 2H), 4.16-4.05 (m, 1H), 3.01-2.85 (m, 2H), 2.47- 2.29 (m, 2H). | 486.2 |
| | | 4-(2,6-difluorobenzyl)-2-(4-((2- | | | |
| | | ((3,3-difluorocyclobutyl)amino) | | | |
| | | pyridin-4-yl)oxy)phenyl)-2,4-dihydro- | | | |
| | | 3H-1,2,4-triazol-3-one | | | |
| Example 329 Method 61 | ++++ | 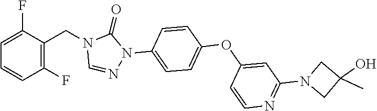 [SMILES: CC1(O)CN(c2cc(Oc3ccc(-n4ncn(Cc5c(F)cccc5F)c4=O)cc3)ccn2)C1] | white solid, yield: 13.8% | ¹H NMR (400 MHz, Chloroform-d) δ 8.02-7.93 (m, 3H), 7.59 (d, J = 0.8 Hz, 1H), 7.37 (tt, J = 8.4, 6.4 Hz, 1H), 7.15-7.07 (m, 2H), 7.04- 6.91 (m, 2H), 6.22 (dd, J = 6.0, 2.2 Hz, 1H), 5.74 (d, J = 2.0 Hz, 1H), 4.98 (d, J = 1.2 Hz, 2H), 3.93- | 466.2 |
| | | 4-(2,6-difluorobenzyl)-2-(4-((2-(3- | | 3.83 (m, 4H), 1.57 (s, 3H). | |
| | | hydroxy-3-methylazetidin-1-yl) | | | |
| | | pyridin-4-yl)oxy)phenyl)-2,4-dihydro- | | | |
| | | 3H-1,2,4-triazol-3-one | | | |
| Example 330 Method 61 | ++++ | 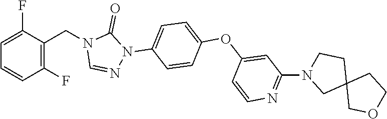 [SMILES: O=c1n(Cc2c(F)cccc2F)cnn1-c1ccc(Oc2ccnc(N3CCC4(CCOC4)C3)c2)cc1] | white solid, yield: 18.3% | ¹H NMR (400 MHz, Chloroform-d) δ 8.04-7.94 (m, 3H), 7.59 (s, 1H), 7.37 (tt, J = 8.4, 6.4 Hz, 1H), 7.17- 7.08 (m, 2H), 6.98 (t, J = 8.0 Hz, 2H), 6.19 (dd, J = 5.6, 2.0 Hz, 1H), 5.80 (d, J = 2.0 Hz, 1H), 4.99 (s, 2H), 3.98-3.84 (m, 2H), 3.74- 3.62 (m, 2H), 3.53-3.33 (m, 4H), | 506.2 |
| | | 2-(4-((2-(2-oxa-7-azaspiro[4.4] | | 2.08-1.84 (m, 6H). | |
| | | nonan-7-yl)pyridin-4-yl)oxy)phenyl)- | | | |
| | | 4-(2,6-difluorobenzyl)-2,4-dihydro- | | | |
| | | 3H-1,2,4-triazol-3-one | | | |
| Example 331 Method 61 | ++++ | 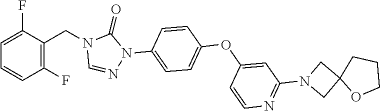 [SMILES: O=c1n(Cc2c(F)cccc2F)cnn1-c1ccc(Oc2ccnc(N3CC4(CCCO4)C3)c2)cc1] | white solid, yield: 27.2% | ¹H NMR (400 MHz, Chloroform-d) δ 8.03-7.93 (m, 3H), 7.59 (d, J = 0.8 Hz, 1H), 7.37 (tt, J = 8.4, 6.4 Hz, 1H), 7.17-7.07 (m, 2H), 7.04- 6.96 (m, 2H), 6.22 (dd, J = 5.6, 2.0 Hz, 1H), 5.75 (d, J = 2.0 Hz, 1H), 4.98 (s, 2H), 4.03-3.97 (m, 2H), 3.95-3.90 (m, 2H), 3.86 (t, J = | 492.2 |
| | | 2-(4-((2-(5-oxa-2-azaspiro[3.4]octan- | | 6.8 Hz, 2H), 2.17-2.10 (m, 2H), | |
| | | 2-yl)pyridin-4-yl)oxy)phenyl)- | | 1.99-1.89 (m, 2H). | |
| | | 4-(2,6-difluorobenzyl)-2,4-dihydro- | | | |
| | | 3H-1,2,4-triazol-3-one | | | |
| Example 332 Method 61 | +++ | 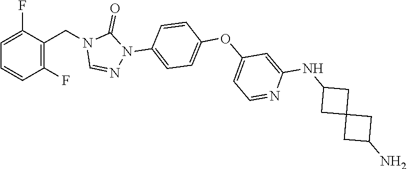 [SMILES: NC1CC2(C1)CC(Nc1cc(Oc3ccc(-n4ncn(Cc5c(F)cccc5F)c4=O)cc3)ccn1)C2] | white solid, yield: 14.5% | ¹H NMR (400 MHz, Chloroform-d) δ 9.38 (brs, 1H), 8.08 (d, J = 8.4 Hz, 2H), 7.69-7.62 (m, 2H), 7.37 (ddd, J = 15.2, 8.4, 6.4 Hz, 1H), 7.14 (d, J = 8.4 Hz, 2H), 7.03- 6.95 (m, 2H), 6.35 (d, J = 7.2 Hz, 1H), 5.81 (s, 1H), 4.98 (s, 2H), 3.77-3.59 (m, 2H), 2.55-2.33 (m, 4H), 2.16-2.01 (m, 4H), 1.42- 1.38 (m, 2H). | 505.2 |
| | | 2-(4-((2-((6-aminospiro[3.3]heptan- | | | |
| | | 2-yl)amino)pyridin-4-yl)oxy)phenyl)- | | | |
| | | 4-(2,6-difluorobenzyl)-2,4-dihydro- | | | |
| | | 3H-1,2,4-triazol-3-one | | | |
| Example 333 Method 61 | ++++ | 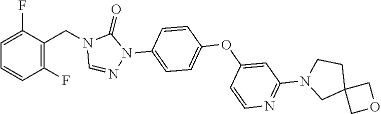 [SMILES: O=c1n(Cc2c(F)cccc2F)cnn1-c1ccc(Oc2ccnc(N3CCC4(COC4)C3)c2)cc1] | white solid, yield: 6.9% | ¹H NMR (400 MHz, Chloroform-d) δ 8.16-8.02 (m, 3H), 7.62 (s, 1H), 7.44-7.32 (m, 1H), 7.18-7.10 (m, 2H), 7.00 (t, J = 8.0 Hz, 2H), 6.47-6.33 (m, 1H), 5.80 (d, J = 2.4 Hz, 1H), 5.00 (s, 2H), 4.79- 4.66 (m, 2H), 4.59 (d, J = 6.4 Hz, 2H), 3.84-3.43 (m, 4H), 2.34 (t, | 492.2 |
| | | 2-(4-((2-(2-oxa-6-azaspiro[3.4]octan- | | J = 6.8 Hz, 2H). | |
| | | 6-yl)pyridin-4-yl)oxy)phenyl)- | | | |
| | | 4-(2,6-difluorobenzyl)-2,4-dihydro- | | | |
| | | 3H-1,2,4-triazol-3-one | | | |
| Example 334 Method 61 | ++++ | 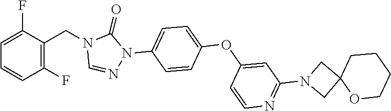 [SMILES: O=c1n(Cc2c(F)cccc2F)cnn1-c1ccc(Oc2ccnc(N3CC4(CCCCO4)C3)c2)cc1] | white solid, yield: 8.2% | ¹H NMR (400 MHz, Chloroform-d) δ 8.00 (dd, J = 7.6, 1.6 Hz, 3H), 7.60 (d, J = 1.2 Hz, 1H), 7.37 (tt, J = 8.4, 6.4 Hz, 1H), 7.16-7.08 (m, 2H), 7.04-6.93 (m, 2H), 6.26 (dd, J = 6.0, 2.0 Hz, 1H), 5.72 (d, J = 2.0 Hz, 1H), 4.99 (d, J = 1.2 Hz, 2H), 3.91-3.86 (m, 4H), 3.68 | 506.2 |
| | | 2-(4-((2-(5-oxa-2-azaspiro[3.5]nonan- | | 3.61 (m, 2H), 1.85-1.77 (m, 2H), | |
| | | 2-yl)pyridin-4-yl)oxy)phenyl)- | | 1.71-1.61 (m, 2H), 1.58-1.49 | |
| | | 4-(2,6-difluorobenzyl)-2,4-dihydro- | | (m, 2H). | |
| | | 3H-1,2,4-triazol-3-one | | | |
| Example 335 Method 61 | ++++ | 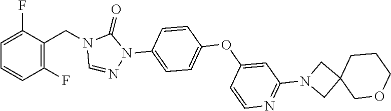 [SMILES: O=c1n(Cc2c(F)cccc2F)cnn1-c1ccc(Oc2ccnc(N3CC4(CCCOC4)C3)c2)cc1] | white solid, yield: 9.3% | ¹H NMR (400 MHz, Chloroform-d) δ 8.07-7.96 (m, 3H), 7.60 (s, 1H), 7.43-7.31 (m, 1H), 7.13 (td, J = 6.4, 2.4 Hz, 2H), 6.99 (t, J = 8.0 Hz, 2H), 6.23 (dd, J = 6.0, 2.0 Hz, 1H), 5.70 (d, J = 2.4 Hz, 1H), 4.99 (s, 2H), 3.79-3.55 (m, 7H), 1.87- 1.79 (m, 2H), 1.37-1.22 (m, 3H). | 506.2 |
| | | 2-(4-((2-(6-oxa-2-azaspiro[3.5]nonan- | | | |
| | | 2-yl)pyridin-4-yl)oxy)phenyl)- | | | |
| | | 4-(2,6-difluorobenzyl)-2,4-dihydro- | | | |
| | | 3H-1,2,4-triazol-3-one | | | |
| Example 336 Method 61 | ++++ | 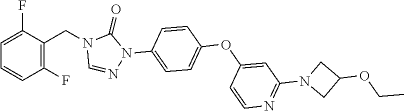 [SMILES: CCOC1CN(c2cc(Oc3ccc(-n4ncn(Cc5c(F)cccc5F)c4=O)cc3)ccn2)C1] | Off- white solid, yield: 7.8% | ¹H NMR (400 MHz, Chloroform-d) δ 8.00 (dd, J = 13.6, 7.6 Hz, 3H), 7.60 (d, J = 0.8 Hz, 1H), 7.37 (tt, J = 8.4, 6.4 Hz, 1H), 7.17-7.08 (m, 2H), 7.05-6.94 (m, 2H), 6.30- 6.25 (m, 1H), 5.73-5.68 (m, 1H), 4.99 (d, J = 1.2 Hz, 2H), 4.45- 4.35 (m, 1H), 4.33-4.13 (m, 2H), | 480.2 |
| | | 4-(2,6-difluorobenzyl)-2-(4-((2-(3- | | 4.02-3.84 (m, 2H), 3.47 (q, J = | |
| | | ethoxyazetidin-1-yl)pyridin-4-yl) | | 7.2 Hz, 2H), 1.21 (t, J = 7.2 Hz, | |
| | | oxy)phenyl)-2,4-dihydro-3H-1,2,4- | | 3H). | |
| | | triazol-3-one | | | |
| Example 337 Method 61 | ++++ | 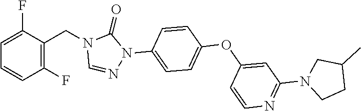 [SMILES: CC1CCN(c2cc(Oc3ccc(-n4ncn(Cc5c(F)cccc5F)c4=O)cc3)ccn2)C1] | white solid, yield: 0.9% | ¹H NMR (400 MHz, Chloroform-d) δ 8.08-7.96 (m, 3H), 7.60 (s, 1H), 7.37 (tt, J = 8.4, 6.4 Hz, 1H), 7.22- 7.09 (m, 2H), 7.04-6.94 (m, 2H), 6.25-6.19 (m, 1H), 5.80 (d, J = 2.0 Hz, 1H), 4.99 (t, J = 1.2 Hz, 2H), 3.65-3.50 (m, 2H), 3.48- 3.36 (m, 1H), 3.03-2.88 (m, 1H), | 464.2 |
| | | 4-(2,6-difluorobenzyl)-2-(4-((2-(3- | | 2.44-2.30 (m, 1H), 2.18-2.06 | |
| | | methylpyrrolidin-1-yl)pyridin-4- | | (m, 1H), 1.68-1.54 (m, 1H), 1.10 | |
| | | yl)oxy)phenyl)-2,4-dihydro-3H- | | (d, J = 6.4 Hz, 3H). | |
| | | 1,2,4-triazol-3-one | | | |
| Example 338 Method 61 | +++ | 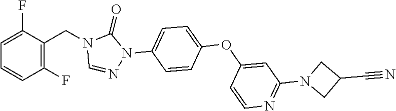 [SMILES: N#CC1CN(c2cc(Oc3ccc(-n4ncn(Cc5c(F)cccc5F)c4=O)cc3)ccn2)C1] | white solid, yield: 8.6% | ¹H NMR (400 MHz, Chloroform-d) δ 8.02 (dd, J = 8.0, 6.0 Hz, 3H), 7.60 (s, 1H), 7.37 (tt, J = 8.4, 6.4 Hz, 1H), 7.16-7.08 (m, 2H), 6.99 (t, J = 8.0 Hz, 2H), 6.34 (dd, J = 6.0, 2.1 Hz, 1H), 5.75 (d, J = 2.0 Hz, 1H), 4.99 (s, 2H), 4.30 (t, J = 8.4 Hz, 2H), 4.21 (t, J = 7.2 Hz, | 461.2 |
| | | 1-(4-(4-(4-(2,6-difluorobenzyl)-5- | | 2H), 3.63-3.52 (m, 1H). | |
| | | oxo-4,5-dihydro-1H-1,2,4-triazol- | | | |
| | | 1-yl)phenoxy)pyridin-2-yl)azetidine- | | | |
| | | 3-carbonitrile | | | |
| Example 339 Method 61 | ++++ | 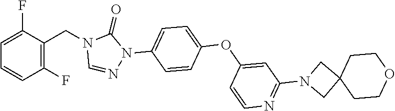 [SMILES: O=c1n(Cc2c(F)cccc2F)cnn1-c1ccc(Oc2ccnc(N3CC4(CCOCC4)C3)c2)cc1] | white solid, yield: 35.3% | ¹H NMR (400 MHz, Chloroform-d) δ 8.03-7.95 (m, 3H), 7.60 (s, 1H), 7.37 (tt, J = 8.4, 6.4 Hz, 1H), 7.16- 7.08 (m, 2H), 7.04-6.94 (m, 2H), 6.25 (dd, J = 6.0, 2.0 Hz, 1H), 5.68 (d, J = 2.0 Hz, 1H), 4.99 (s, 2H), 3.73 (s, 4H), 3.64 (t, J = 5.2 Hz, 4H), 1.80 (t, J = 5.2 Hz, 4H). | 506.2 |
| | | 2-(4-((2-(7-oxa-2-azaspiro[3.5]nonan- | | | |
| | | 2-yl)pyridin-4-yl)oxy)phenyl)- | | | |
| | | 4-(2,6-difluorobenzyl)-2,4-dihydro- | | | |
| | | 3H-1,2,4-triazol-3-one | | | |
| Example 340 Method 61 | ++++ | 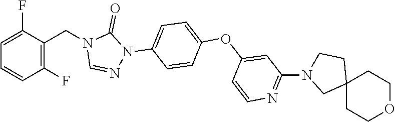 [SMILES: O=c1n(Cc2c(F)cccc2F)cnn1-c1ccc(Oc2ccnc(N3CCC4(CCOCC4)C3)c2)cc1] | white solid, yield: 29.2% | ¹H NMR (400 MHz, Chloroform-d) δ 8.07-7.93 (m, 3H), 7.60 (s, 1H), 7.37 (tt, J = 8.4, 6.4 Hz, 1H), 7.16- 7.10 (m, 2H), 7.05-6.95 (m, 2H), 6.18 (dd, J = 5.6, 2.0 Hz, 1H), 5.82 (d, J = 2.0 Hz, 1H), 4.99 (t, J = 1.2 Hz, 2H), 3.74 (dt, J = 11.6, 5.2 Hz, 2H), 3.66 (dt, J = 11.6, 5.6 | 520.2 |
| | | 2-(4-((2-(8-oxa-2-azaspiro[4.5]decan- | | Hz, 2H), 3.41 (t, J = 7.2 Hz, 2H), | |
| | | 2-yl)pyridin-4-yl)oxy)phenyl)- | | 3.36 (s, 2H), 1.89 (t, J = 7.2 Hz, | |
| | | 4-(2,6-difluorobenzyl)-2,4-dihydro- | | 2H), 1.62 (t, J = 5.6 Hz, 4H). | |
| | | 3H-1,2,4-triazol-3-one | | | |
| Example 341 Method 61 | ++ | 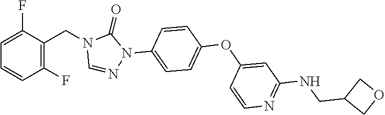 [SMILES: O=c1n(Cc2c(F)cccc2F)cnn1-c1ccc(Oc2ccnc(NCC3COC3)c2)cc1] | yellow oil, yield: 1.7% | ¹H NMR (400 MHz, Chloroform-d) δ 9.23 (brs, 1H), 8.07-8.01 (m, 3H), 7.60 (s, 1H), 7.48-7.32 (m, 1H), 7.12 (s, 2H), 6.98 (t, J = 7.6 Hz, 2H), 6.38 (s, 2H), 5.03-4.90 (m, 2H), 4.32-4.02 (m, 2H), 3.80- 3.28 (m, 4H), 2.52-2.39 (m, 1H). | 466.2 |
| | | 4-(2,6-difluorobenzyl)-2-(4-((2- | | | |
| | | ((oxetan-3-ylmethyl)amino)pyridin- | | | |
| | | 4-yl)oxy)phenyl)-2,4-dihydro-3H- | | | |
| | | 1,2,4-triazol-3-one | | | |
| Example 342 Method 61 | +++ | 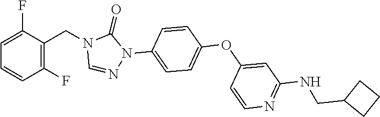 [SMILES: O=c1n(Cc2c(F)cccc2F)cnn1-c1ccc(Oc2ccnc(NCC3CCC3)c2)cc1] | white solid, yield: 12.2% | ¹H NMR (400 MHz, Chloroform-d) δ 9.15 (brs, 1H), 8.15-8.09 (m, 2H), 7.70-7.65 (m, 1H), 7.63 (s, 1H), 7.39 (ddd, J = 14.8, 8.4, 6.4 Hz, 1H), 7.16 (d, J = 8.8 Hz, 2H), 7.00 (dd, J = 8.4, 7.2 Hz, 2H), 6.34 (d, J = 6.0 Hz, 1H), 5.91 (d, J = 2.0 Hz, 1H), 5.00 (s, 2H), 3.07 (d, J = | 464.2 |
| | | 2-(4-((2-((cyclobutylmethyl)amino) | | 7.2 Hz, 2H), 2.65-2.57 (m, 1H), | |
| | | pyridin-4-yl)oxy)phenyl)-4-(2,6- | | 2.16-2.06 (m, 2H), 1.98-1.81 | |
| | | difluorobenzyl)-2,4-dihydro-3H- | | (m, 2H), 1.74-1.63 (m, 2H). | |
| | | 1,2,4-triazol-3-one | | | |
| Example 343 Method 61 | ++ | 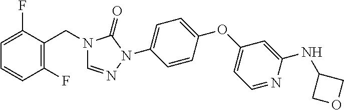 [SMILES: O=c1n(Cc2c(F)cccc2F)cnn1-c1ccc(Oc2ccnc(NC3COC3)c2)cc1] | white solid, yield: 2.6% | ¹H NMR (400 MHz, Chloroform-d) δ 10.68 (brs, 1H), 8.14 (d, J = 8.8 Hz, 2H), 7.71 (d, J = 7.2 Hz, 1H), 7.65-7.58 (m, 1H), 7.38 (p, J = 7.2 Hz, 1H), 7.16-7.12 (m, 2H), 7.00 (t, J = 8.0 Hz, 2H), 6.40 (dd, J = 7.2, 2.0 Hz, 1H), 5.66 (d, J = 2.4 | 452.2 |
| | | 4-(2,6-difluorobenzyl)-2-(4-((2- | | Hz, 1H), 5.00 (s, 2H), 4.79 (t, J = | |
| | | (oxetan-3-ylamino)pyridin-4-yl)oxy) | | 6.8 Hz, 2H), 4.71 (t, J = 6.4 Hz, | |
| | | phenyl)-2,4-dihydro-3H-1,2,4- | | 2H), 4.47 (q, J = 6.4 Hz, 1H). | |
| | | triazol-3-one | | | |
| Example 344 Method 61 | +++ | 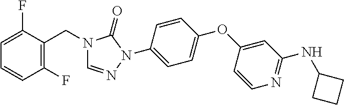 [SMILES: O=c1n(Cc2c(F)cccc2F)cnn1-c1ccc(Oc2ccnc(NC3CCC3)c2)cc1] | white solid, yield: 29.5% | ¹H NMR (400 MHz, Chloroform-d) δ 9.39 (brs, 1H), 8.12 (d, J = 8.8 Hz, 2H), 7.74-7.58 (m, 2H), 7.44- 7.33 (m, 1H), 7.16 (d, J = 8.8 Hz, 2H), 7.00 (t, J = 8.0 Hz, 2H), 6.35 (s, 1H), 5.79 (s, 1H), 5.00 (s, 2H), 3.81-3.67 (m, 1H), 2.35-2.02 | 450.2 |
| | | 2-(4-((2-(cyclobutylamino)pyridin- | | (m, 4H), 1.92-1.65 (m, 2H). | |
| | | 4-yl)oxy)phenyl)-4-(2,6-difluoro- | | | |
| | | benzyl)-2,4-dihydro-3H-1,2,4- | | | |
| | | triazol-3-one | | | |
| Example 345 Method 61 | +++ | 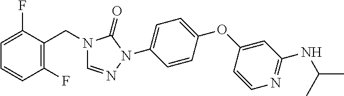 [SMILES: CC(C)Nc1cc(Oc2ccc(-n3ncn(Cc4c(F)cccc4F)c3=O)cc2)ccn1] | white solid, yield: 17.4% | ¹H NMR (400 MHz, Chloroform-d) δ 9.27 (brs, 1H), 8.15-8.07 (m, 2H), 7.70-7.57 (m, 2H), 7.38 (tt, J = 8.4, 6.4 Hz, 1H), 7.20-7.13 (m, 2H), 7.02-6.97 (m, 2H), 6.31 (d, J = 5.6 Hz, 1H), 5.91 (d, J = 2.0 Hz, 1H), 5.00 (s, 2H), 3.44 (s, 1H), | 438.2 |
| | | 4-(2,6-difluorobenzyl)-2-(4-((2-(iso- | | 1.25 (d, J = 6.0 Hz, 6H). | |
| | | propylamino)pyridin-4-yl)oxy) | | | |
| | | phenyl)-2,4-dihydro-3H-1,2,4-triazol- | | | |
| | | 3-one | | | |
| Example 346 Method 61 | +++ | 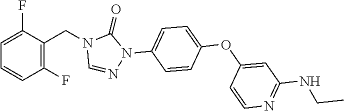 [SMILES: CCNc1cc(Oc2ccc(-n3ncn(Cc4c(F)cccc4F)c3=O)cc2)ccn1] | white solid, yield: 48.6% | ¹H NMR (400 MHz, Chloroform-d) δ 9.20 (brs, 1H), 8.15-8.08 (m, 2H), 7.67 (d, J = 7.2 Hz, 1H), 7.63 (s, 1H), 7.38 (ddd, J = 14.8, 8.4, 6.4 Hz, 1H), 7.20-7.13 (m, 2H), 7.00 (dd, J = 8.4, 7.2 Hz, 2H), 6.38- 6.32 (m, 1H), 5.90 (d, J = 2.0 Hz, 1H), 5.00 (s, 2H), 3.11 (d, J = 7.6 | 424.2 |
| | | 4-(2,6-difluorobenzyl)-2-(4-((2- | | Hz, 2H), 1.26 (t, J = 7.2 Hz, 3H). | |
| | | (ethylamino)pyridin-4-yl)oxy)phenyl)- | | | |
| | | 2,4-dihydro-3H-1,2,4-triazol-3-one | | | |
| Example 347 Method 61 | ++++ | 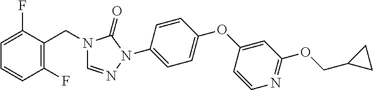 [SMILES: O=c1n(Cc2c(F)cccc2F)cnn1-c1ccc(Oc2ccnc(OCC3CC3)c2)cc1] | light pink solid, yield: 12.0% | ¹H NMR (400 MHz, Chloroform-d) δ 8.03-7.95 (m, 3H), 7.59 (s, 1H), 7.43-7.31 (m, 1H), 7.13 (d, J = 8.4 Hz, 2H), 6.99 (t, J = 8.0 Hz, 2H), 6.51 (d, J = 5.6 Hz, 1H), 6.19 (s, 1H), 4.99 (s, 2H), 4.08 (d, J = | 451.2 |
| | | 2-(4-((2-(cyclopropylmethoxy)pyridin- | | 7.2 Hz, 2H), 1.28-1.17 (m, 1H), | |
| | | 4-yl)oxy)phenyl)-4-(2,6-difluoro- | | 0.58 (d, J = 7.6 Hz, 2H), 0.31 (d, | |
| | | benzyl)-2,4-dihydro-3H-1,2,4- | | J = 5.2 Hz, 2H). | |
| | | triazol-3-one | | | |
| Example 348 Method 61: | +++ | 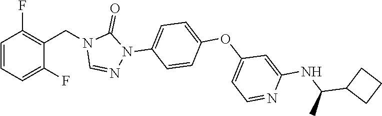 [SMILES: C[C@@H](Nc1cc(Oc2ccc(-n3ncn(Cc4c(F)cccc4F)c3=O)cc2)ccn1)C1CCC1] | white solid, yield: 15.7% | ¹H NMR (400 MHz, DMSO-d₆) δ 12.74 (brs, 1H), 8.35 (s, 1H), 8.02- 7.96 (m, 2H), 7.87 (d, J = 6.8 Hz, 1H), 7.49 (tt, J = 8.4, 6.8 Hz, 1H), 7.37-7.31 (m, 2H), 7.22-7.12 (m, 2H), 6.52 (s, 1H), 6.04 (s, 1H), 4.99 (s, 2H), 2.33-2.22 (m, 1H), 2.01-1.84 (m, 2H), 1.78-1.58 | 478.2 |
| | | (R)-2-(4-((2-((1-cyclobutylethyl) | | (m, 2H), 1.39-1.18 (m, 3H), 0.99 | |
| | | amino)pyridin-4-yl)oxy)phenyl)-4- | | (d, J = 6.3 Hz, 3H). | |
| | | (2,6-difluorobenzyl)-2,4-dihydro- | | | |
| | | 3H-1,2,4-triazol-3-one | | | |
| Example 349 Method 61: | +++ | 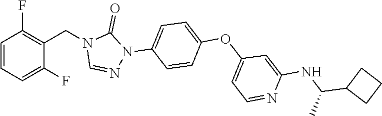 [SMILES: C[C@H](Nc1cc(Oc2ccc(-n3ncn(Cc4c(F)cccc4F)c3=O)cc2)ccn1)C1CCC1] | white solid, yield: 9.6% | ¹H NMR (400 MHz, DMSO-d₆) δ 8.35 (s, 1H), 8.01-7.95 (m, 2H), 7.87 (d, J = 6.8 Hz, 1H), 7.49 (tt, J = 8.5, 6.8 Hz, 1H), 7.33 (d, J = 8.8 Hz, 2H), 7.22-7.12 (m, 2H), 6.49 (s, 1H), 6.02 (s, 1H), 5.00 (s, 2H), 2.29 (d, J = 8.0 Hz, 1H), 2.03- 1.57 (m, 6H), 1.23 (s, 1H), 0.98 (d, | 478.2 |
| | | (S)-2-(4-((2-((1-cyclobutylethyl) | | J = 6.4 Hz, 3H). | |
| | | amino)pyridin-4-yl)oxy)phenyl)-4- | | | |
| | | (2,6-difluorobenzyl)-2,4-dihydro- | | | |
| | | 3H-1,2,4-triazol-3-one | | | |
| Example 350 Method 61: | ++++ | 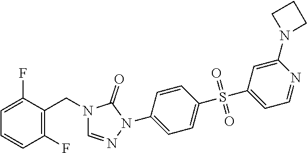 [SMILES: O=c1n(Cc2c(F)cccc2F)cnn1-c1ccc(S(=O)(=O)c2ccnc(N3CCC3)c2)cc1] | white solid, yield: 23.8% | ¹H NMR (400 MHz, DMSO-d₆) δ 8.42 (s, 1H), 8.23 (d, J = 5.2 Hz, 1H), 8.18-8.11 (m, 2H), 8.10- 8.03 (m, 2H), 7.54-7.42 (m, 1H), 7.16 (t, J = 8.0 Hz, 2H), 6.93 (dd, J = 5.2, 1.6 Hz, 1H), 6.72 (d, J = 1.6 Hz, 1H), 4.98 (s, 2H), 4.00 (t, J = 7.6 Hz, 4H), 2.37-2.29 (m, 2H). | 484.2 |
| | | 2-(4-((2-(azetidin-1-yl)pyridin-4-yl) | | | |
| | | sulfonyl)phenyl)-4-(2,6-difluoro- | | | |
| | | benzyl)-2,4-dihydro-3H-1,2,4-triazol- | | | |
| | | 3-one | | | |
| Example 351 Method 61: | +++ | 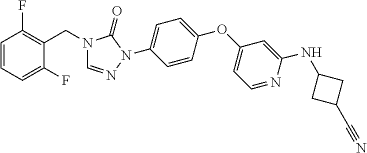 [SMILES: N#CC1CC(Nc2cc(Oc3ccc(-n4ncn(Cc5c(F)cccc5F)c4=O)cc3)ccn2)C1] | white solid, yield: 26.6% | ¹H NMR (400 MHz, DMSO-d₆) δ 8.35 (s, 1H), 8.02-7.96 (m, 2H), 7.94 (t, J = 6.4 Hz, 1H), 7.49 (tt, J = 8.4, 6.8 Hz, 1H), 7.33 (dd, J = 8.8, 4.6 Hz, 2H), 7.17 (t, J = 8.0 Hz, 2H), 6.59 (d, J = 24.0 Hz, 1H), 6.01 (d, J = 11.6 Hz, 1H), 5.00 (s, 2H), 4.22-4.11 (m, 1H), 3.09 (m, J = 9.1 Hz, 1H), 2.74 (q, J = 11.2, 6.8 Hz, 2H), 2.21 (q, J = 10.0 Hz, | 475.2 |
| | | 3-((4-(4-(4-(2,6-difluorobenzyl)-5- | | 2H). | |
| | | oxo-4,5-dihydro-1H-1,2,4-triazol- | | | |
| | | 1-yl)phenoxy)pyridin-2-yl)amino) | | | |
| | | cyclobutane-1-carbonitrile | | | |
| Example 352 Method 61: | +++ | 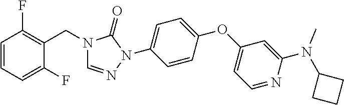 [SMILES: CN(c1cc(Oc2ccc(-n3ncn(Cc4c(F)cccc4F)c3=O)cc2)ccn1)C1CCC1] | yellow solid, yield: 2.1% | ¹H NMR (400 MHz, DMSO-d₆) δ 8.34 (s, 1H), 7.98 (d, J = 8.0 Hz, 3H), 7.49 (p, J = 7.6 Hz, 1H), 7.31 (d, J = 8.4 Hz, 2H), 7.17 (t, J = 8.0 Hz, 2H), 6.43 (d, J = 6.4 Hz, 1H), 6.33 (s, 1H), 4.99 (s, 2H), 4.47 (p, J = 8.6 Hz, 1H), 2.99 (s, 3H), 2.14 | 464.2 |
| | | 2-(4-((2-(cyclobutyl(methyl)amino) | | (t, J = 8.8 Hz, 4H), 1.69-1.54 (m, | |
| | | pyridin-4-yl)oxy)phenyl)-4-(2,6- | | 2H). | |
| | | difluorobenzyl)-2,4-dihydro-3H- | | | |
| | | 1,2,4-triazol-3-one | | | |
| Example 353 Method 61: | +++ | 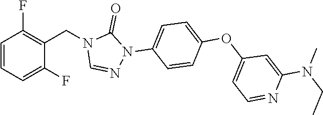 [SMILES: CCN(C)c1cc(Oc2ccc(-n3ncn(Cc4c(F)cccc4F)c3=O)cc2)ccn1] | yellow solid, yield: 10.4% | ¹H NMR (400 MHz, DMSO-d₆) δ 8.32 (s, 1H), 8.06-7.82 (m, 3H), 7.53-7.47 (m, 1H), 7.26-7.06 (m, 4H), 6.08 (dt, J = 6.4, 2.0 Hz, 2H), 4.99 (s, 2H), 3.50 (q, J = 7.2 Hz, 2H), 2.90 (s, 3H), 1.02 (t, J = 7.2 Hz, 3H). | 438.2 |
| | | 4-(2,6-difluorobenzyl)-2-(4-((2- | | | |
| | | (ethyl(methyl)amino)pyridin-4-yl)oxy) | | | |
| | | phenyl)-2,4-dihydro-3H-1,2,4- | | | |
| | | triazol-3-one | | | |
| Example 354 Method 61: | +++ | 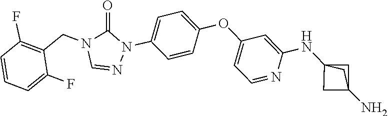 [SMILES: NC12CC(Nc3cc(Oc4ccc(-n5ncn(Cc6c(F)cccc6F)c5=O)cc4)ccn3)(C1)C2] | white solid, yield: 2.6% | ¹H NMR (400 MHz, DMSO-d₆) δ 8.61 (s, 3H), 8.34 (s, 1H), 7.99- 7.91 (m, 3H), 7.31-7.24 (m, 2H), 7.17 (t, J = 8.0 Hz, 2H), 6.36 (s, 1H), 5.96 (s, 1H), 4.99 (s, 2H), 2.24 (s, 6H). | 477.2 |
| | | 2-(4-((2-((3-aminobicyclo[1.1.1] | | | |
| | | pentan-1-yl)amino)pyridin-4-yl)oxy) | | | |
| | | phenyl)-4-(2,6-difluorobenzyl)- | | | |
| | | 2,4-dihydro-3H-1,2,4-triazol-3-one | | | |
| Example 355 Method 61: | ++++ | 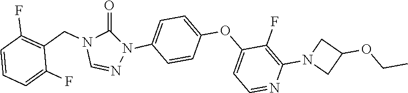 [SMILES: CCOC1CN(c2nccc(Oc3ccc(-n4ncn(Cc5c(F)cccc5F)c4=O)cc3)c2F)C1] | white solid, yield: 30.8% | ¹H NMR (400 MHz, DMSO-d₆) δ 8.32 (s, 1H), 7.94-7.87 (m, 2H), 7.83-7.75 (m, 1H), 7.55-7.43 (m, 1H), 7.27-7.12 (m, 5H), 6.27 (td, J = 5.6, 1.4 Hz, 1H), 4.98 (d, J = 5.2 Hz, 3H), 4.44-4.36 (m, 1H), | 498.2 |
| | | 4-(2,6-difluorobenzyl)-2-(4-((2-(3- | | 4.27 (t, J = 7.6 Hz, 2H), 3.89 (dd, | |
| | | ethoxyazetidin-1-yl)-3-fluoropyridin- | | J = 9.2, 4.4 Hz, 2H), 3.51-3.42 (m, | |
| | | 4-yl)oxy)phenyl)-2,4-dihydro- | | 2H), 1.18-1.04 (m, 3H). | |
| | | 3H-1,2,4-triazol-3-one | | | |
| Example 356 Method 61: | ++++ | 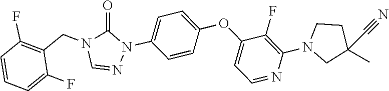 [SMILES: CC1(C#N)CCN(c2nccc(Oc3ccc(-n4ncn(Cc5c(F)cccc5F)c4=O)cc3)c2F)C1] | white solid, yield: 5.5% | ¹H NMR (400 MHz, DMSO-d₆) δ 8.32 (s, 1H), 7.88 (s, 1H), 7.81 (dd, J = 5.6, 1.6 Hz, 1H), 7.55-7.43 (m, 1H), 7.28-7.21 (m, 2H), 7.17 (t, J = 8.2 Hz, 2H), 6.27 (dd, J = 6.4, 4.5 Hz, 1H), 4.99 (s, 2H), 4.02 | 507.2 |
| | | 1-(4-(4-(4-(2,6-difluorobenzyl)-5- | | (dd, J = 10.8, 2.5 Hz, 1H), 3.78- | |
| | | oxo-4,5-dihydro-1H-1,2,4-triazol- | | 3.74 (m, 2H), 3.53 (dd, J = 10.8, | |
| | | 1-yl)phenoxy)-3-fluoropyridin-2- | | 1.6 Hz, 1H), 2.40 (dt, J = 11.6, 5.6 | |
| | | yl)-3-methylpyrrolidine-3-carbonitrile | | Hz, 1H), 2.13-1.97 (m, 1H), 1.51 | |
| | | | | (d, J = 2.0 Hz, 3H). | |
| Example 357 Method 61: | ++++ | 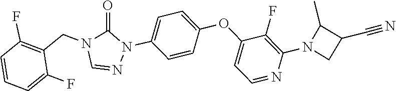 [SMILES: CC1C(C#N)CN1c1nccc(Oc2ccc(-n3ncn(Cc4c(F)cccc4F)c3=O)cc2)c1F] | white solid, yield: 4.3% | ¹H NMR (400 MHz, DMSO-d₆) δ 8.32 (d, J = 2.4 Hz, 1H), 7.91 (dt, J = 10.0, 2.8 Hz, 2H), 7.85 (dd, J = 5.6, 2.6 Hz, 1H), 7.48-7.47 (m, 1H), 7.25 (dd, J = 9.2, 2.8 Hz, 2H), 7.17 (td, J = 8.0, 2.6 Hz, 2H), 6.37 (td, J = 5.6, 2.4 Hz, 1H), 4.98 (s, | 493.2 |
| | | 1-(4-(4-(4-(2,6-difluorobenzyl)-5- | | 2H), 4.73-4.65 (m, 1H), 4.30- | |
| | | oxo-4,5-dihydro-1H-1,2,4-triazol- | | 4.22 (m, 1H), 4.14 (d, J = 7.6 Hz, | |
| | | 1-yl)phenoxy)-3-fluoropyridin-2- | | 1H), 3.94 (q, J = 6.4, 4.0 Hz, 1H), | |
| | | yl)-2-methylazetidine-3-carbonitrile | | 1.52 (dd, J = 6.4, 2.4 Hz, 3H). | |
| Example 358 Method 61: | ++++ | 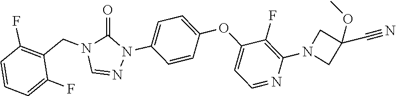 [SMILES: COC1(C#N)CN(c2nccc(Oc3ccc(-n4ncn(Cc5c(F)cccc5F)c4=O)cc3)c2F)C1] | white solid, yield: 4.9% | ¹H NMR (400 MHz, DMSO-d₆) δ 8.32 (s, 1H), 7.95-7.88 (m, 2H), 7.85 (d, J = 5.6 Hz, 1H), 7.55- 7.43 (m, 1H), 7.29-7.22 (m, 2H), 7.17 (t, J = 8.0 Hz, 2H), 6.40 (t, J = 5.5 Hz, 1H), 4.99 (s, 2H), 4.55- | 509.2 |
| | | 1-(4-(4-(4-(2,6-difluorobenzyl)-5- | | 4.48 (m, 2H), 4.25 (d, J = 9.6 Hz, | |
| | | oxo-4,5-dihydro-1H-1,2,4-triazol- | | 2H), 3.44 (s, 3H). | |
| | | 1-yl)phenoxy)-3-fluoropyridin-2- | | | |
| | | yl)-3-methoxyazetidine-3-carbonitrile | | | |
| Example 359 Method 61: SIR- 000081 | +++ | 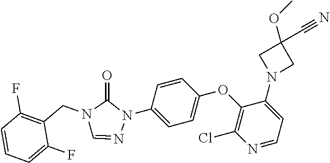 [SMILES: COC1(C#N)CN(c2ccnc(Cl)c2Oc2ccc(-n3ncn(Cc4c(F)cccc4F)c3=O)cc2)C1] | white solid, yield: 10.7% | ¹H NMR (400 MHz, DMSO-d₆) δ 8.28 (s, 1H), 8.06 (d, J = 5.6 Hz, 1H), 7.87-7.76 (m, 2H), 7.48 (tt, J = 8.4, 6.8 Hz, 1H), 7.08 (d, J = 5.4 Hz, 1H), 7.04-6.88 (m, 2H), 4.97 (s, 2H), 4.40 (dd, J = 9.6, 1.2 Hz, 2H), 4.17-4.10 (m, 2H), 3.35 (s, 3H). | 526.0 |
| | | 1-(2-chloro-3-(4-(4-(2,6-difluoro- | | | |
| | | benzyl)-5-oxo-4,5-dihydro-1H-1,2,4- | | | |
| | | triazol-1-yl)phenoxy)pyridin-4- | | | |
| | | yl)-3-methoxyazetidine-3-carbonitrile | | | |
| Example 360 Method 61: | ++ | 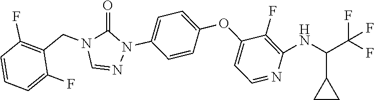 [SMILES: O=c1n(Cc2c(F)cccc2F)cnn1-c1ccc(Oc2ccnc(NC(C3CC3)C(F)(F)F)c2F)cc1] | white solid, yield: 3.9% | ¹H NMR (400 MHz, DMSO-d₆) δ 8.32 (s, 1H), 7.95-7.88 (m, 2H), 7.71 (d, J = 5.6 Hz, 1H), 7.55- 7.38 (m, 2H), 7.30-7.24 (m, 2H), 7.17 (t, J = 8.0 Hz, 2H), 6.19 (t, J = 5.6 Hz, 1H), 4.99 (s, 2H), 4.35 (p, J = 8.4 Hz, 1H), 1.30 (dd, J = 9.6, | 536.2 |
| | | 2-(4-((2-((1-cyclopropyl-2,2,2-trifluoro- | | 4.7 Hz, 1H), 0.72-0.63 (m, 1H), | |
| | | ethyl)amino)-3-fluoropyridin- | | 0.52 (q, J = 8.0, 6.8 Hz, 2H), 0.31- | |
| | | 4-yl)oxy)phenyl)-4-(2,6-difluoro- | | 0.24 (m, 1H). | |
| | | benzyl)-2,4-dihydro-3H-1,2,4-triazol- | | | |
| | | 3-one | | | |
| Example 361 Method 61: | +++ | 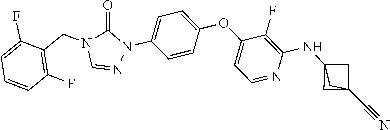 [SMILES: N#CC12CC(Nc3nccc(Oc4ccc(-n5ncn(Cc6c(F)cccc6F)c5=O)cc4)c3F)(C1)C2] | white solid, yield: 6.8% | ¹H NMR (400 MHz, Chloroform-d) δ 8.09-7.98 (m, 2H), 7.94 (d, J = 6.0 Hz, 1H), 7.61 (d, J = 0.8 Hz, 1H), 7.48-7.30 (m, 1H), 7.22- 7.11 (m, 2H), 7.04-6.87 (m, 2H), 6.30 (dd, J = 6.0, 2.4 Hz, 1H), 5.95 (d, J = 2.0 Hz, 1H), 4.99 (t, J = 1.2 Hz, 2H), 2.52 (s, 6H). | 505.2 |
| | | 3-((4-(4-(4-(2,6-difluorobenzyl)-5- | | | |
| | | oxo-4,5-dihydro-1H-1,2,4-triazol- | | | |
| | | 1-yl)phenoxy)-3-fluoropyridin-2- | | | |
| | | yl)amino)bicyclo[1.1.1]pentane-1- | | | |
| | | carbonitrile | | | |
| Example 362 Method 61: | ++ | 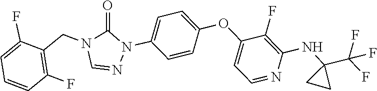 [SMILES: O=c1n(Cc2c(F)cccc2F)cnn1-c1ccc(Oc2ccnc(NC3(C(F)(F)F)CC3)c2F)cc1] | white solid, yield: 2.7% | ¹H NMR (400 MHz, DMSO-d₆) δ 8.32 (s, 1H), 7.95-7.88 (m, 2H), 7.71 (d, J = 5.6 Hz, 1H), 7.55- 7.38 (m, 2H), 7.30-7.24 (m, 2H), 7.17 (t, J = 8.0 Hz, 2H), 6.19 (t, J = 5.8 Hz, 1H), 4.99 (s, 2H), 0.72- | 522.3 |
| | | 4-(2,6-difluorobenzyl)-2-(4-((3- | | 0.63 (m, 1H), 0.52 (q, J = 8.0, 6.8 | |
| | | fluoro-2-((1-(trifluoromethyl)cyclo- | | Hz, 2H), 0.31-0.24 (m, 1H). | |
| | | propyl)amino)pyridin-4-yl)oxy) | | | |
| | | phenyl)-2,4-dihydro-3H-1,2,4-triazol- | | | |
| | | 3-one | | | |
| Example 363 Method 61: | ++++ | 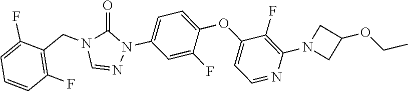 [SMILES: CCOC1CN(c2nccc(Oc3ccc(-n4ncn(Cc5c(F)cccc5F)c4=O)cc3F)c2F)C1] | yellow solid, yield: 19.4% | ¹H NMR (400 MHz, DMSO-d₆) δ 8.32 (s, 1H), 7.94-7.87 (m, 2H), 7.83-7.75 (m, 1H), 7.55-7.43 (m, 1H), 7.27-7.12 (m, 4H), 6.27 (td, J = 5.6, 1.2 Hz, 1H), 4.98 (d, J = 5.2 Hz, 3H), 4.44-4.36 (m, 1H), | 516.3 |
| | | 4-(2,6-difluorobenzyl)-2-(4-((2-(3- | | 4.27 (t, J = 7.6 Hz, 2H), 3.89 (dd, | |
| | | ethoxyazetidin-1-yl)-3-fluoropyridin- | | J = 9.2, 4.4 Hz, 2H), 3.51-3.42 (m, | |
| | | 4-yl)oxy)-3-fluorophenyl)-2,4- | | 2H), 1.18-1.04 (m, 3H). | |
| | | dihydro-3H-1,2,4-triazol-3-one | | | |
| Example 364 Method 61: | ++++ | 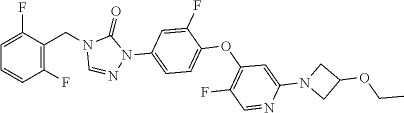 [SMILES: CCOC1CN(c2cc(Oc3ccc(-n4ncn(Cc5c(F)cccc5F)c4=O)cc3F)c(F)cn2)C1] | white solid, yield: 7.6% | ¹H NMR (400 MHz, DMSO-d₆) δ 8.38 (s, 1H), 7.91 (dd, J = 12.4, 2.4 Hz, 1H), 7.79-7.73 (m, 2H), 7.55- 7.40 (m, 2H), 7.22-7.12 (m, 2H), 6.23 (t, J = 5.6 Hz, 1H), 4.99 (s, 2H), 4.41 (tt, J = 6.4, 4.4 Hz, 1H), 4.29-4.27 (m, 2H), 3.93- 3.85 (m, 2H), 3.44 (q, J = 7.2 Hz, | 516.2 |
| | | 4-(2,6-difluorobenzyl)-2-(4-((2-(3- | | 2H), 1.14 (t, J = 7.2 Hz, 3H). | |
| | | ethoxyazetidin-1-yl)-5-fluoropyridin- | | | |
| | | 4-yl)oxy)-3-fluorophenyl)-2,4- | | | |
| | | dihydro-3H-1,2,4-triazol-3-one | | | |
| Example 365 Method 61: | ++++ | 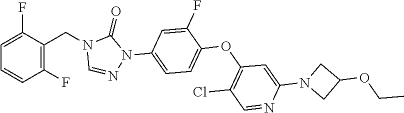 [SMILES: CCOC1CN(c2cc(Oc3ccc(-n4ncn(Cc5c(F)cccc5F)c4=O)cc3F)c(Cl)cn2)C1] | yellow solid, yield: 19.6% | ¹H NMR (400 MHz, DMSO-d₆) δ 8.39 (s, 1H), 8.13 (s, 1H), 7.93 (dd, J = 12.4, 2.4 Hz, 1H), 7.83-7.76 (m, 1H), 7.57-7.41 (m, 2H), 7.22- 7.11 (m, 2H), 5.64 (s, 1H), 5.00 (s, 2H), 4.30 (tt, J = 6.4, 4.0 Hz, 1H), 4.03 (dd, J = 9.2, 6.4 Hz, 2H), 3.64 (dd, J = 9.2, 4.0 Hz, 2H), 3.37 | 532.2 |
| | | 2-(4-((5-chloro-2-(3-ethoxyazetidin- | | (q, J = 7.2 Hz, 2H), 1.09 (t, J = 7.2 | |
| | | 1-yl)pyridin-4-yl)oxy)-3-fluoro- | | Hz, 3H). | |
| | | phenyl)-4-(2,6-difluorobenzyl)-2,4- | | | |
| | | dihydro-3H-1,2,4-triazol-3-one | | | |
| Example 366 Method 61: | ++++ | 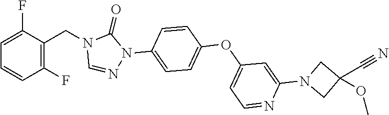 [SMILES: COC1(C#N)CN(c2cc(Oc3ccc(-n4ncn(Cc5c(F)cccc5F)c4=O)cc3)ccn2)C1] | white solid, yield: 12.7% | ¹H NMR (400 MHz, DMSO-d₆) δ 8.32 (s, 1H), 8.02 (d, J = 5.6 Hz, 1H), 7.97-7.89 (m, 2H), 7.49 (tt, J = 8.4, 6.8 Hz, 1H), 7.28-7.11 (m, 4H), 6.34 (dd, J = 5.6, 2.0 Hz, 1H), 5.98 (d, J = 2.0 Hz, 1H), 4.99 (s, 2H), 4.34 (dd, J = 9.2, 1.2 Hz, 2H), 4.05 (dd, J = 9.2, 1.0 Hz, 2H), 3.40 | 491.2 |
| | | 1-(4-(4-(4-(2,6-difluorobenzyl)-5- | | (s, 3H). | |
| | | oxo-4,5-dihydro-1H-1,2,4-triazol- | | | |
| | | 1-yl)phenoxy)pyridin-2-yl)-3- | | | |
| | | methoxyazetidine-3-carbonitrile | | | |
| Example 367 Method 61: | ++++ | 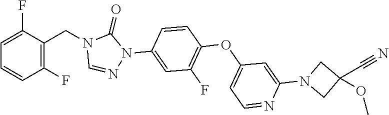 [SMILES: COC1(C#N)CN(c2cc(Oc3ccc(-n4ncn(Cc5c(F)cccc5F)c4=O)cc3F)ccn2)C1] | white solid, yield: 16.9% | ¹H NMR (400 MHz, DMSO-d₆) δ 8.39 (s, 1H), 8.02 (d, J = 5.6 Hz, 1H), 7.91 (dd, J = 12.4, 2.4 Hz, 1H), 7.78-7.69 (m, 1H), 7.55- 7.39 (m, 2H), 7.17 (t, J = 8.0 Hz, 2H), 6.35 (dd, J = 5.8, 2.0 Hz, 1H), 5.99 (d, J = 2.0 Hz, 1H), 5.00 (s, 2H), 4.35 (dd, J = 9.6, 1.2 Hz, 2H), | 509.2 |
| | | 1-(4-(4-(4-(2,6-difluorobenzyl)-5- | | 4.06 (dd, J = 9.6, 1.2 Hz, 2H), 3.40 | |
| | | oxo-4,5-dihydro-1H-1,2,4-triazol- | | (s, 3H). | |
| | | 1-yl)-2-fluorophenoxy)pyridin-2- | | | |
| | | yl)-3-methoxyazetidine-3-carbonitrile | | | |
| Example 368 Method 61: | +++ | 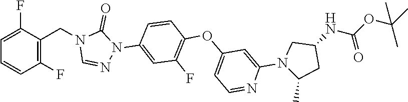 [SMILES: C[C@H]1C[C@@H](NC(=O)OC(C)(C)C)CN1c1cc(Oc2ccc(-n3ncn(Cc4c(F)cccc4F)c3=O)cc2F)ccn1] | Yellow solid, yield: 26.1% | ¹H NMR (400 MHz, DMSO-d₆) δ 8.32 (s, 1H), 7.99-7.88 (m, 3H), 7.49 (tt, J = 8.4, 6.8 Hz, 1H), 7.25- 7.07 (m, 5H), 6.14 (dd, J = 6.0, 2.0 Hz, 1H), 5.87 (s, 1H), 4.98 (s, 2H), 3.94 (q, J = 6.4 Hz, 1H), 3.67 (t, J = 9.2 Hz, 1H), 3.10 (d, J = | 579.4 |
| | | tert-butyl | | 17.6 Hz, 1H), 1.61 (dt, J = 13.2, 6.8 | |
| | | ((3R,5S)-1-(4-(4-(4-(2,6-difluoro- | | Hz, 1H), 1.38 (s, 9H), 1.19 (d, J = | |
| | | benzyl)-5-oxo-4,5-dihydro-1H-1,2, | | 6.0 Hz, 3H). | |
| | | 4-triazol-1-yl)-2-fluorophenoxy) | | | |
| | | pyridin-2-yl)-5-methylpyrrolidin-3- | | | |
| | | yl)carbamate | | | |
| Example 369 Method 61: | ++++ | 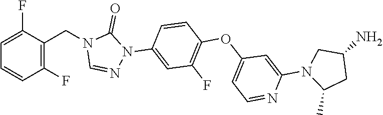 [SMILES: C[C@H]1C[C@@H](N)CN1c1cc(Oc2ccc(-n3ncn(Cc4c(F)cccc4F)c3=O)cc2F)ccn1] | yellow solid, yield: 80.2% | ¹H NMR (400 MHz, DMSO-d₆) δ 8.32 (s, 1H), 7.99-7.88 (m, 3H), 7.49 (tt, J = 8.4, 6.8 Hz, 1H), 7.25- 7.07 (m, 5H), 6.14 (dd, J = 6.0, 2.0 Hz, 1H), 5.87 (s, 1H), 4.98 (s, 2H), 3.94 (q, J = 6.4 Hz, 1H), 3.67 (t, J = 9.0 Hz, 1H), 3.10 (d, J = 17.2 Hz, 1H), 1.61 (dt, J = 13.2, 6.8 | 479.4 |
| | | 2-(4-((2-((2S,4R)-4-amino-2-methyl- | | Hz, 1H), 1.19 (d, J = 6.0 Hz, 3H). | |
| | | pyrrolidin-1-yl)pyridin-4-yl)oxy)- | | | |
| | | 3-fluorophenyl)-4-(2,6-difluoro- | | | |
| | | benzyl)-2,4-dihydro-3H-1,2,4-triazol- | | | |
| | | 3-one | | | |
| Example 370 Method 61: | ++++ | 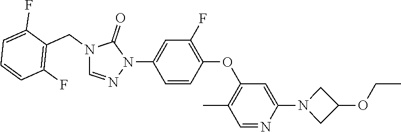 [SMILES: CCOC1CN(c2cc(Oc3ccc(-n4ncn(Cc5c(F)cccc5F)c4=O)cc3F)c(C)cn2)C1] | white solid, yield: 18.6% | ¹H NMR (400 MHz, DMSO-d₆) δ 8.39 (s, 1H), 7.95-7.87 (m, 2H), 7.80-7.73 (m, 1H), 7.55-7.43 (m, 1H), 7.38 (t, J = 9.0 Hz, 1H), 7.22-7.12 (m, 2H), 5.50 (s, 1H), 4.99 (s, 2H), 4.29 (tt, J = 6.4, 4.2 Hz, 1H), 3.97 (dd, J = 8.8, 6.4 Hz, 2H), 3.55 (dd, J = 8.8, 4.4 Hz, 2H), 3.36 (q, J = 7.2 Hz, 2H), 2.12 (s, | 512.2 |
| | | 4-(2,6-difluorobenzyl)-2-(4-((2-(3- | | 3H), 1.08 (t, J = 7.2 Hz, 3H). | |
| | | ethoxyazetidin-1-yl)-5-methyl- | | | |
| | | pyridin-4-yl)oxy)-3-fluorophenyl)- | | | |
| | | 2,4-dihydro-3H-1,2,4-triazol-3-one | | | |
| Example 371 Method 61: | +++ | 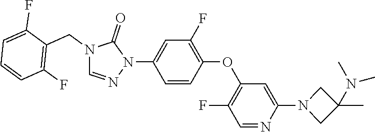 [SMILES: CN(C)C1(C)CN(c2cc(Oc3ccc(-n4ncn(Cc5c(F)cccc5F)c4=O)cc3F)c(F)cn2)C1] | yellow solid, yield: 31.1% | ¹H NMR (400 MHz, DMSO-d₆) δ 8.39 (d, J = 7.2 Hz, 1H), 8.10 (d, J = 3.2 Hz, 1H), 7.93 (dd, J = 12.4, 2.4 Hz, 1H), 7.78 (ddd, J = 9.2, 2.4, 1.2 Hz, 1H), 7.55-7.43 (m, 2H), 7.22-7.11 (m, 2H), 5.70 (d, J = 5.6 Hz, 1H), 5.00 (s, 2H), 3.56 (d, J = 7.6 Hz, 2H), 3.47 (d, J = 7.6 Hz, 2H), 1.18 (s, 3H). | 529.2 |
| | | 4-(2,6-difluorobenzyl)-2-(4-((2-(3- | | | |
| | | (dimethylamino)-3-methylazetidin- | | | |
| | | 1-yl)-5-fluoropyridin-4-yl)oxy)- | | | |
| | | 3-fluorophenyl)-2,4-dihydro-3H- | | | |
| | | 1,2,4-triazol-3-one | | | |
| Example 372 Method 61: | ++++ | 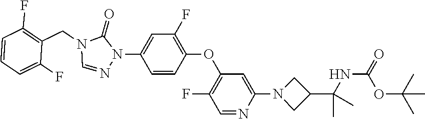 [SMILES: CC(C)(C)OC(=O)NC(C)(C)C1CN(c2cc(Oc3ccc(-n4ncn(Cc5c(F)cccc5F)c4=O)cc3F)c(F)cn2)C1] | yellow solid, yield: 19.2% | ¹H NMR (400 MHz, DMSO-d₆) δ 8.38 (s, 1H), 8.12 (d, J = 2.8 Hz, 1H), 7.93 (dd, J = 12.4, 2.4 Hz, 1H), 7.78 (ddd, J = 9.2, 2.6, 1.2 Hz, 1H), 7.55-7.42 (m, 2H), 7.22- 7.13 (m, 2H), 6.97 (t, J = 5.6 Hz, 1H), 5.84 (d, J = 5.6 Hz, 1H), 4.99 (s, 2H), 4.03 (d, J = 6.0 Hz, 1H), | 629.2 |
| | | tert-butyl | | 3.76-3.68 (m, 1H), 3.57 (q, J = | |
| | | (2-(1-(4-(4-(4-(2,6-difluorobenzyl)- | | 8.0 Hz, 1H), 3.05 (dt, J = 13.2, 6.4 | |
| | | 5-oxo-4,5-dihydro-1H-1,2,4-triazol- | | Hz, 1H), 2.51 (s, 6H), 2.22 (s, 2H), | |
| | | 1-yl)-2-fluorophenoxy)-5-fluoro- | | 2.16-1.97 (m, 1H), 1.42-1.32 | |
| | | pyridin-2-yl)azetidin-3-yl)propan- | | (m, 2H), 1.26 (s, 9H). | |
| | | 2-yl)carbamate | | | |
| Example 373 Method 61: | ++++ | 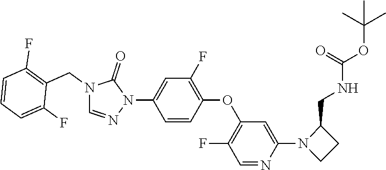 [SMILES: CC(C)(C)OC(=O)NC[C@H]1CCN1c1cc(Oc2ccc(-n3ncn(Cc4c(F)cccc4F)c3=O)cc2F)c(F)cn1] | Yellow solid, yield: 16.3% | ¹H NMR (400 MHz, DMSO-d₆) δ 8.39 (s, 1H), 8.10 (d, J = 3.2 Hz, 1H), 7.93 (dd, J = 12.4, 2.4 Hz, 1H), 7.82-7.75 (m, 1H), 7.55- 7.43 (m, 2H), 7.22-7.11 (m, 2H), 6.47 (s, 1H), 5.66 (d, J = 5.6 Hz, 1H), 4.99 (s, 2H), 3.71 (t, J = 8.4 Hz, 2H), 3.62 (dd, J = 8.4, 6.0 Hz, 2H), 3.02-2.90 (m, 1H), 1.32 (s, 9H). | 601.2 |
| | | tert-butyl | | | |
| | | (R)-((1-(4-(4-(4-(2,6-difluorobenzyl)- | | | |
| | | 5-oxo-4,5-dihydro-1H-1,2,4-triazol- | | | |
| | | 1-yl)-2-fluorophenoxy)-5-fluoro- | | | |
| | | pyridin-2-yl)azetidin-2-yl)methyl) | | | |
| | | carbamate | | | |
| Example 374 Method 61: | +++ | 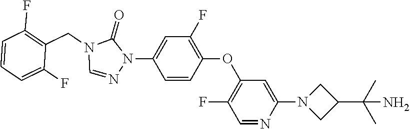 [SMILES: CC(C)(N)C1CN(c2cc(Oc3ccc(-n4ncn(Cc5c(F)cccc5F)c4=O)cc3F)c(F)cn2)C1] | white solid, yield: 16.0% | ¹H NMR (400 MHz, DMSO-d₆) δ 8.41 (s, 1H), 8.18 (s, 1H), 7.95 (dd, J = 12.4, 2.4 Hz, 1H), 7.84-7.77 (m, 1H), 7.56-7.43 (m, 2H), 7.22- 7.13 (m, 2H), 5.68 (s, 1H), 5.00 (s, 2H), 3.83 (d, J = 23.2 Hz, 10H), 2.85 (s, 1H). | 529.2 |
| | | 2-(4-((2-(3-(2-aminopropan-2-yl) | | | |
| | | azetidin-1-yl)-5-fluoropyridin-4-yl) | | | |
| | | oxy)-3-fluorophenyl)-4-(2,6-difluoro- | | | |
| | | benzyl)-2,4-dihydro-3H-1,2,4- | | | |
| | | triazol-3-one | | | |
| Example 375 Method 61: | ++++ | 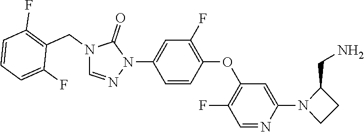 [SMILES: NC[C@H]1CCN1c1cc(Oc2ccc(-n3ncn(Cc4c(F)cccc4F)c3=O)cc2F)c(F)cn1] | white solid, yield: 56.7% | ¹H NMR (400 MHz, DMSO-d₆) δ 8.41 (s, 1H), 8.19 (d, J = 3.2 Hz, 1H), 8.11 (s, 3H), 7.98-7.90 (m, 1H), 7.83-7.77 (m, 1H), 7.59- 7.44 (m, 2H), 7.22-7.12 (m, 2H), 5.82 (d, J = 5.6 Hz, 1H), 5.00 (s, 2H), 4.39 (t, J = 6.0 Hz, 1H), 3.76- 3.63 (m, 1H), 3.62-3.44 (m, 1H), 3.09 (q, J = 5.6 Hz, 2H), 2.30- | 501.2 |
| | | (R)-2-(4-((2-(2-(aminomethyl) | | 2.20 (m, 2H). | |
| | | azetidin-1-yl)-5-fluoropyridin-4-yl) | | | |
| | | oxy)-3-fluorophenyl)-4-(2,6-difluoro- | | | |
| | | benzyl)-2,4-dihydro-3H-1,2,4- | | | |
| | | triazol-3-one | | | |
| Example 376 Method 61: | ++++ | 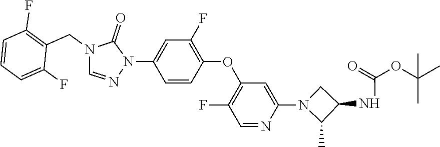 [SMILES: C[C@H]1[C@H](NC(=O)OC(C)(C)C)CN1c1cc(Oc2ccc(-n3ncn(Cc4c(F)cccc4F)c3=O)cc2F)c(F)cn1] | white solid, yield: 25.7% | ¹H NMR (400 MHz, DMSO-d₆) δ 8.40 (s, 1H), 8.22-8.18 (m, 1H), 7.94 (dd, J = 12.4, 2.4 Hz, 1H), 7.84-7.73 (m, 1H), 7.55-7.43 (m, 2H), 7.17 (t, J = 8.0 Hz, 2H), 5.86 (d, J = 5.6 Hz, 1H), 5.00 (s, 2H), 4.19 (s, 1H), 4.08-4.03 (m, 1H), 3.74-3.60 (m, 2H), 1.37 (d, J = 6.0 Hz, 3H), 1.26 (s, 9H). | 601.2 |
| | | tert-butyl | | | |
| | | ((2S,3R)-1-(4-(4-(4-(2,6-difluoro- | | | |
| | | benzyl)-5-oxo-4,5-dihydro-1H-1,2,4- | | | |
| | | triazol-1-yl)-2-fluorophenoxy)- | | | |
| | | 5-fluoropyridin-2-yl)-2-methyl- | | | |
| | | azetidin-3-yl)carbamate | | | |
| Example 377 Method 61: | ++++ | 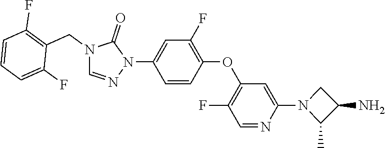 [SMILES: C[C@H]1[C@H](N)CN1c1cc(Oc2ccc(-n3ncn(Cc4c(F)cccc4F)c3=O)cc2F)c(F)cn1] | white solid, yield: 55.7% | ¹H NMR (400 MHz, DMSO-d₆) δ 8.40 (s, 1H), 8.22-8.18 (m, 1H), 7.94 (dd, J = 12.4, 2.6 Hz, 1H), 7.84-7.73 (m, 1H), 7.55-7.43 (m, 2H), 7.17 (t, J = 8.0 Hz, 2H), 5.86 (d, J = 5.6 Hz, 1H), 5.00 (s, 2H), 4.19 (s, 1H), 4.08-4.03 (m, 1H), 3.74-3.60 (m, 2H), 1.37 (d, J = 6.0 Hz, 3H). | 501.2 |
| | | 2-(4-((2-((2S,3R)-3-amino-2-methyl- | | | |
| | | azetidin-1-yl)-5-fluoropyridin-4- | | | |
| | | yl)oxy)-3-fluorophenyl)-4-(2,6-di- | | | |
| | | fluorobenzyl)-2,4-dihydro-3H-1,2,4- | | | |
| | | triazol-3-one | | | |
| Example 378 Method 61: | +++ | 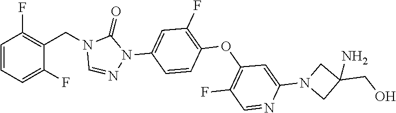 [SMILES: NC1(CO)CN(c2cc(Oc3ccc(-n4ncn(Cc5c(F)cccc5F)c4=O)cc3F)c(F)cn2)C1] | yellow solid, yield: 88.4% | ¹H NMR (400 MHz, DMSO-d₆) δ 8.60 (s, 3H), 8.41 (s, 1H), 8.22 (d, J = 3.2 Hz, 1H), 7.94 (dd, J = 12.4, 2.4 Hz, 1H), 7.83-7.76 (m, 1H), 7.57-7.43 (m, 2H), 7.17 (t, J = 8.0 Hz, 2H), 5.95 (d, J = 5.6 Hz, 1H), 5.00 (s, 2H), 3.96-3.85 (m, 4H), 3.75-3.59 (m, 2H). | 517.2 |
| | | 2-(4-((2-(3-amino-3-(hydroxy- | | | |
| | | methyl)azetidin-1-yl)-5-fluoropyridin- | | | |
| | | 4-yl)oxy)-3-fluorophenyl)-4-(2,6- | | | |
| | | difluorobenzyl)-2,4-dihydro-3H- | | | |
| | | 1,2,4-triazol-3-one | | | |
| Example 379 Method 61: | +++ | 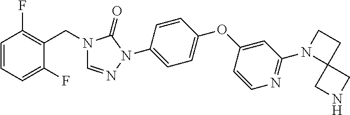 [SMILES: O=c1n(Cc2c(F)cccc2F)cnn1-c1ccc(Oc2ccnc(N3CCC34CNC4)c2)cc1] | Light- yellow solid, yield: 66.6% | ¹H NMR (400 MHz, DMSO) δ 8.34 (s, 1H), 8.07 (d, J = 6.0 Hz, 1H), 8.00-7.91 (m, 2H), 7.55-7.44 (m, 1H), 7.29-7.21 (m, 2H), 7.17 (t, J = 8.1 Hz, 2H), 6.31 (dd, J = 6.0, 2.2 Hz, 1H), 5.97 (s, 1H), 4.99 (s, 2H), 4.64 (dt, J = 11.1, 5.5 Hz, 2H), 4.13 (t, J = 9.5 Hz, 2H), 3.72 (t, J = | 477.3 |
| | | 2-(4-((2-(1,6-diazaspiro[3.3]heptan- | | 7.0 Hz, 2H), 2.58 (t, J = 7.0 Hz, | |
| | | 1-yl)pyridin-4-yl)oxy)phenyl)-4- | | 2H). | |
| | | (2,6-difluorobenzyl)-2,4-dihydro- | | | |
| | | 3H-1,2,4-triazol-3-one | | | |
| Example 380 Method 61: | +++ | 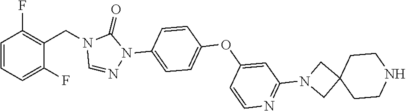 [SMILES: O=c1n(Cc2c(F)cccc2F)cnn1-c1ccc(Oc2ccnc(N3CC4(CCNCC4)C3)c2)cc1] | Light- yellow solid, yield: 62.6% | NA | 505.2 |
| | | 2-[4-[[2-(2,7-diazaspiro[3.5]nonan- | | | |
| | | 2-yl)-4-pyridyl]oxy]phenyl]-4- | | | |
| | | [(2,6-difluorophenyl)methyl]-1,2,4- | | | |
| | | triazol-3-one | | | |
| Example 381 Method 61 | ++++ | 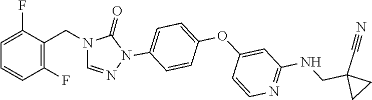 [SMILES: N#CC1(CNc2cc(Oc3ccc(-n4ncn(Cc5c(F)cccc5F)c4=O)cc3)ccn2)CC1] | Light yellow solid, yield: 9.7% | ¹H NMR (400 MHz, Chloroform-d) δ 8.03-7.95 (m, 2H), 7.92 (d, J = 6.0 Hz, 1H), 7.60 (s, 1H), 7.37 (tt, J = 8.4, 6.4 Hz, 1H), 7.17-7.08 (m, 2H), 7.04-6.94 (m, 2H), 6.26 (dd, J = 6.0, 2.0 Hz, 1H), 5.90 (d, J = 2.0 Hz, 1H), 4.99 (s, 2H), 4.79 (brs, | 475.2 |
| | | 1-(((4-(4-(4-(2,6-difluorobenzyl)- | | 1H), 3.51 (d, J = 6.0 Hz, 2H), 1.28- | |
| | | 5-oxo-4,5-dihydro-1H-1,2,4-triazol- | | 1.15 (m, 2H), 1.15-1.03 (m, 2H). | |
| | | 1-yl)phenoxy)pyridin-2-yl)amino) | | | |
| | | methyl)cyclopropane-1-carbonitrile | | | |
| Example 382 Method 61 | +++ | 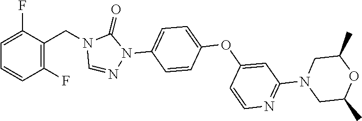 [SMILES: C[C@@H]1CN(c2cc(Oc3ccc(-n4ncn(Cc5c(F)cccc5F)c4=O)cc3)ccn2)C[C@H](C)O1] | white solid, yield: 19.5% | ¹H NMR (400 MHz, Chloroform-d) δ 8.13 (dd, J = 8.4, 6.4 Hz, 3H), 7.63 (d, J = 0.8 Hz, 1H), 7.38 (tt, J = 8.4, 6.4 Hz, 1H), 7.19-7.11 (m, 2H), 7.05-6.98 (m, 2H), 6.47 (dd, J = 7.2, 2.0 Hz, 1H), 6.14 (d, J = 2.0 Hz, 1H), 5.00 (d, J = 1.2 Hz, 2H), 3.88 (d, J = 12.8 Hz, 2H), | 494.2 |
| | | 4-(2,6-difluorobenzyl)-2-(4-((2- | | 3.78-3.66 (m, 2H), 2.77 (dd, J = | |
| | | ((2R,6S)-2,6-dimethylmorpholino) | | 12.8, 10.4 Hz, 2H), 1.25 (d, J = 6.0 | |
| | | pyridin-4-yl)oxy)phenyl)-2,4-dihydro- | | Hz, 6H). | |
| | | 3H-1,2,4-triazol-3-one | | | |
| Example 383 Method 61 | ++++ | 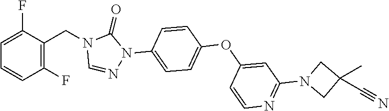 [SMILES: CC1(C#N)CN(c2cc(Oc3ccc(-n4ncn(Cc5c(F)cccc5F)c4=O)cc3)ccn2)C1] | white solid, yield: 27.3% | ¹H NMR (400 MHz, Chloroform-d) δ 8.05-7.97 (m, 3H), 7.60 (s, 1H), 7.37 (tt, J = 8.4, 6.4 Hz, 1H), 7.16- 7.07 (m, 2H), 7.04-6.93 (m, 2H), 6.31 (dd, J = 5.6, 2.0 Hz, 1H), 5.74 (d, J = 2.0 Hz, 1H), 4.99 (d, J = 1.2 Hz, 2H), 4.30 (d, J = 7.6 Hz, | 475.2 |
| | | 1-(4-(4-(4-(2,6-difluorobenzyl)-5- | | 2H), 3.87 (d, J = 8.0 Hz, 2H), 1.73 | |
| | | oxo-4,5-dihydro-1H-1,2,4-triazol- | | (s, 3H). | |
| | | 1-yl)phenoxy)pyridin-2-yl)-3- | | | |
| | | methylazetidine-3-carbonitrile | | | |
| Example 384 Method 61 | ++++ | 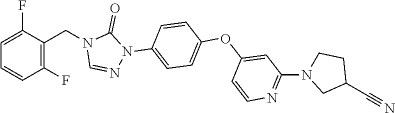 [SMILES: N#CC1CCN(c2cc(Oc3ccc(-n4ncn(Cc5c(F)cccc5F)c4=O)cc3)ccn2)C1] | white solid, yield: 7.3% | ¹H NMR (400 MHz, Chloroform-d) δ 8.06-7.96 (m, 3H), 7.60 (d, J = 0.8 Hz, 1H), 7.37 (tt, J = 8.4, 6.4 Hz, 1H), 7.17-7.09 (m, 2H), 7.04- 6.93 (m, 2H), 6.26 (dd, J = 6.0, 2.0 Hz, 1H), 5.85 (d, J = 2.0 Hz, 1H), 4.99 (t, J = 1.2 Hz, 2H), 3.86- 3.77 (m, 1H), 3.77-3.69 (m, | 475.2 |
| | | 1-(4-(4-(4-(2,6-difluorobenzyl)-5- | | 1H), 3.65-3.55 (m, 1H), 3.52- | |
| | | oxo-4,5-dihydro-1H-1,2,4-triazol- | | 3.42 (m, 1H), 3.27-3.16 (m, 1H), | |
| | | 1-yl)phenoxy)pyridin-2-yl)pyrrolidine- | | 2.46-2.27 (m, 2H). | |
| | | 3-carbonitrile | | | |
| Example 385 Method 61 | ++ | 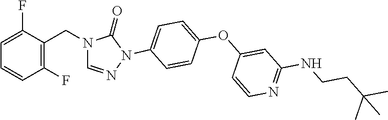 [SMILES: CC(C)(C)CCNc1cc(Oc2ccc(-n3ncn(Cc4c(F)cccc4F)c3=O)cc2)ccn1] | white solid, yield: 5.7% | ¹H NMR (400 MHz, Chloroform-d) δ 8.00 (d, J = 8.4 Hz, 2H), 7.91- 7.85 (m, 1H), 7.59 (s, 1H), 7.37 (t, J = 7.6 Hz, 1H), 7.13 (d, J = 8.4 Hz, 2H), 7.03-6.95 (m, 2H), 6.18 (d, J = 6.0 Hz, 1H), 5.87 (s, 1H), 4.99 (s, 2H), 3.20-3.10 (m, 2H), 1.54-1.45 (m, 2H), 0.92 (d, J = | 480.2 |
| | | 4-(2,6-difluorobenzyl)-2-(4-((2- | | 2.8 Hz, 9H). | |
| | | ((3,3-dimethylbutyl)amino)pyridin- | | | |
| | | 4-yl)oxy)phenyl)-2,4-dihydro-3H- | | | |
| | | 1,2,4-triazol-3-one | | | |
| Example 386 Method 61 | ++++ | 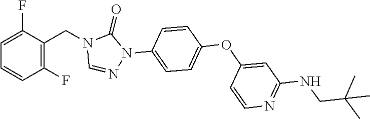 [SMILES: CC(C)(C)CNc1cc(Oc2ccc(-n3ncn(Cc4c(F)cccc4F)c3=O)cc2)ccn1] | light yellow solid, yield: 18.1% | ¹H NMR (400 MHz, Chloroform-d) δ 7.98 (d, J = 8.4 Hz, 2H), 7.94- 7.88 (m, 1H), 7.59 (s, 1H), 7.43- 7.32 (m, 1H), 7.12 (d, J = 8.4 Hz, 2H), 6.98 (t, J = 8.0 Hz, 2H), 6.20- 6.14 (m, 1H), 5.86 (s, 1H), 4.98 (s, 2H), 4.61 (brs, 1H), 3.00-2.94 (m, 2H), 0.95 (s, 9H). | 466.2 |
| | | 4-(2,6-difluorobenzyl)-2-(4-((2- | | | |
| | | (neopentylamino)pyridin-4-yl)oxy) | | | |
| | | phenyl)-2,4-dihydro-3H-1,2,4-triazol- | | | |
| | | 3-one | | | |
| Example 387 Method 61 | +++ | 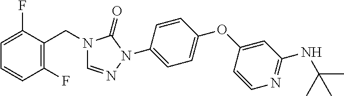 [SMILES: CC(C)(C)Nc1cc(Oc2ccc(-n3ncn(Cc4c(F)cccc4F)c3=O)cc2)ccn1] | Off- white solid, yield: 2.8% | ¹H NMR (400 MHz, Chloroform-d) δ 8.00 (d, J = 8.4 Hz, 2H), 7.87 (d, J = 5.2 Hz, 1H), 7.60 (s, 1H), 7.41-7.33 (m, 1H), 7.13 (d, J = 8.4 Hz, 2H), 7.04- 6.94 (m, 2H), 6.20-6.14 (m, 1H), 5.97 (s, 1H), 4.99 (s, 2H), 1.36 (d, | 452.2 |
| | | 2-(4-((2-(tert-butylamino)pyridin- | | J = 3.2 Hz, 9H). | |
| | | 4-yl)oxy)phenyl)-4-(2,6-difluoro- | | | |
| | | benzyl)-2,4-dihydro-3H-1,2,4-triazol- | | | |
| | | 3-one | | | |
| Example 388 Method 61 | +++ | 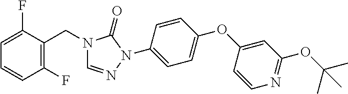 [SMILES: CC(C)(C)Oc1cc(Oc2ccc(-n3ncn(Cc4c(F)cccc4F)c3=O)cc2)ccn1] | white solid, yield: 5.7% | ¹H NMR (400 MHz, Chloroform-d) δ 8.02 (d, J = 9.2 Hz, 3H), 7.60 (s, 1H), 7.43-7.31 (m, 1H), 7.13 (d, J = 8.4 Hz, 2H), 6.99 (t, J = 8.4 Hz, 2H), 6.53 (s, 1H), 6.15 (s, 1H), 4.99 (s, 2H), 1.55 (s, 9H). | 453.2 |
| | | 2-(4-((2-(tert-butoxy)pyridin-4-yl) | | | |
| | | oxy)phenyl)-4-(2,6-difluorobenzyl)- | | | |
| | | 2,4-dihydro-3H-1,2,4-triazol-3-one | | | |
| Example 389 Method 61: | ++++ | 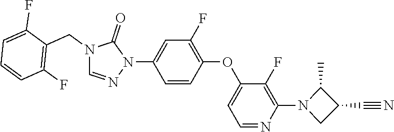 [SMILES: C[C@@H]1[C@H](C#N)CN1c1nccc(Oc2ccc(-n3ncn(Cc4c(F)cccc4F)c3=O)cc2F)c1F] | white solid, yield: 13.5% | ¹H NMR (400 MHz, DMSO-d₆) δ 8.32 (s, 1H), 7.96-7.87 (m, 2H), 7.84 (d, J = 5.6 Hz, 1H), 7.49 (tt, J = 8.4, 6.4 Hz, 1H), 7.29-7.21 (m, 2H), 7.21-7.11 (m, 2H), 6.36 (t, J = 5.6 Hz, 1H), 4.98 (s, 2H), 4.74- 4.63 (m, 1H), 4.26 (td, J = 8.8, 2.4 Hz, 1H), 4.14 (ddd, J = 8.0, 4.8, 2.4 Hz, 1H), 3.94 (td, J = 8.6, 4.6 Hz, | 493.6 |
| | | (2R,3R)-1-(4-(4-(4-(2,6-difluoro- | | 1H), 1.52 (d, J = 6.4 Hz, 3H). | |
| | | benzyl)-5-oxo-4,5-dihydro-1H-1,2,4- | | | |
| | | triazol-1-yl)-2-fluorophenoxy)- | | | |
| | | 3-fluoropyridin-2-yl)-2-methyl- | | | |
| | | azetidine-3-carbonitrile | | | |
| Example 390 Method 61: | ++++ | 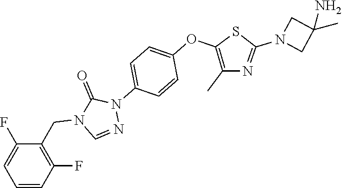 [SMILES: Cc1nc(N2CC(C)(N)C2)sc1Oc1ccc(-n2ncn(Cc3c(F)cccc3F)c2=O)cc1] | white solid, yield: 30.0% | ¹H NMR (400 MHz, DMSO-d₆) δ 8.28 (s, 1H), 7.86-7.77 (m, 2H), 7.48 (tt, J = 8.4, 6.8 Hz, 1H), 7.21- 7.01 (m, 4H), 4.97 (s, 2H), 3.75 (s, 4H), 1.98 (s, 3H), 1.41 (s, 3H) | 485.3 |
| | | 2-(4-((2-(3-amino-3-methylazetidin- | | | |
| | | 1-yl)-4-methylthiazol-5-yl)oxy) | | | |
| | | phenyl)-4-(2,6-difluorobenzyl)-2,4- | | | |
| | | dihydro-3H-1,2,4-triazol-3-one | | | |
| Example 391 Method 61: | +++ | 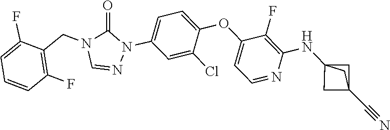 [SMILES: N#CC12CC(Nc3nccc(Oc4ccc(-n5ncn(Cc6c(F)cccc6F)c5=O)cc4Cl)c3F)(C1)C2] | white solid, yield: 16.7% | ¹H NMR (400 MHz, DMSO-d₆) δ 8.38 (s, 1H), 8.11 (d, J = 2.4 Hz, 1H), 7.87 (dd, J = 9.2, 2.4 Hz, 1H), 7.74 (d, J = 6.0 Hz, 2H), 7.60- 7.46 (m, 1H), 7.41 (d, J = 9.2 Hz, 1H), 7.22-7.11 (m, 2H), 6.09 (t, J = 5.6 Hz, 1H), 4.99 (s, 2H), 2.57 (s, 6H). | 539.4 |
| | | 3-((4-(2-chloro-4-(4-(2,6-difluoro- | | | |
| | | benzyl)-5-oxo-4,5-dihydro-1H-1,2,4- | | | |
| | | triazol-1-yl)phenoxy)-3-fluoro- | | | |
| | | pyridin-2-yl)amino)bicyclo[1.1.1] | | | |
| | | pentane-1-carbonitrile | | | |
| Example 392 Method 61: | ++++ | 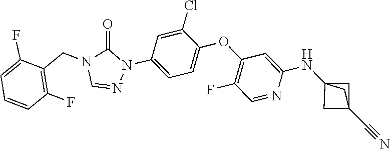 [SMILES: N#CC12CC(Nc3cc(Oc4ccc(-n5ncn(Cc6c(F)cccc6F)c5=O)cc4Cl)c(F)cn3)(C1)C2] | Yellow solid, yield: 15.6% | ¹H NMR (400 MHz, DMSO-d₆) δ 8.40 (s, 1H), 8.13 (d, J = 2.4 Hz, 1H), 8.07 (s, 1H), 7.92 (dd, J = 8.8, 2.4 Hz, 1H), 7.56-7.44 (m, 4H), 7.17 (t, J = 8.0 Hz, 2H), 5.67 (s, 1H), 5.00 (s, 2H), 2.46 (s, 6H). | 539.4 |
| | | 3-((4-(2-chloro-4-(4-(2,6-difluoro- | | | |
| | | benzyl)-5-oxo-4,5-dihydro-1H-1,2,4- | | | |
| | | triazol-1-yl)phenoxy)-5-fluoro- | | | |
| | | pyridin-2-yl)amino)bicyclo[1.1.1] | | | |
| | | pentane-1-carbonitrile | | | |
| Example 393 Method 61: | ++++ | 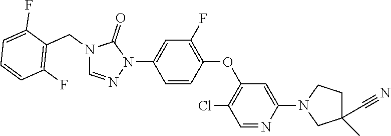 [SMILES: CC1(C#N)CCN(c2cc(Oc3ccc(-n4ncn(Cc5c(F)cccc5F)c4=O)cc3F)c(Cl)cn2)C1] | white solid, yield: 17.2% | ¹H NMR (400 MHz, DMSO-d₆) δ 8.40 (s, 1H), 8.16 (s, 1H), 7.94 (dd, J = 12.4, 2.4 Hz, 1H), 7.80 (ddd, J = 9.2, 2.4, 1.2 Hz, 1H), 7.55-7.42 (m, 2H), 7.22-7.12 (m, 2H), 5.75 (d, J = 4.8 Hz, 1H), 5.00 (s, 2H), 3.78 (d, J = 10.8 Hz, 1H), 3.39 (q, J = 8.4, 7.6 Hz, 1H), 3.24 (d, J = 10.8 Hz, 1H), 2.41-2.32 (m, 1H), | 541.0 |
| | | 1-(5-chloro-4-(4-(4-(2,6-difluoro- | | 2.10-1.97 (m, 1H), 1.43 (s, 3H), | |
| | | benzyl)-5-oxo-4,5-dihydro-1H-1,2,4- | | 1.28-1.13 (m, 1H). | |
| | | triazol-1-yl)-2-fluorophenoxy) | | | |
| | | pyridin-2-yl)-3-methylpyrrolidine- | | | |
| | | 3-carbonitrile | | | |
| Example 394 Method 61: | ++++ | 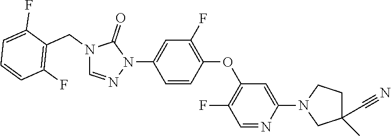 [SMILES: CC1(C#N)CCN(c2cc(Oc3ccc(-n4ncn(Cc5c(F)cccc5F)c4=O)cc3F)c(F)cn2)C1] | white solid, yield: 19.5% | ¹H NMR (400 MHz, DMSO-d₆) δ 8.40 (s, 1H), 8.16 (d, J = 2.8 Hz, 1H), 7.94 (dd, J = 12.4, 2.4 Hz, 1H), 7.82-7.75 (m, 1H), 7.55- 7.43 (m, 2H), 7.21-7.12 (m, 2H), 5.85 (d, J = 5.6 Hz, 1H), 5.00 (s, 2H), 3.78 (d, J = 10.8 Hz, 1H), 3.44-3.34 (m, 1H), 3.23 (d, J = 10.8 Hz, 1H), 2.40-2.32 (m, 1H), | 525.2 |
| | | 1-(4-(4-(4-(2,6-difluorobenzyl)-5- | | 2.10-1.97 (m, 1H), 1.44 (s, 3H). | |
| | | oxo-4,5-dihydro-1H-1,2,4-triazol- | | | |
| | | 1-yl)-2-fluorophenoxy)-5-fluoro- | | | |
| | | pyridin-2-yl)-3-methylpyrrolidine- | | | |
| | | 3-carbonitrile | | | |
| Example 395 Method 61: | ++ | 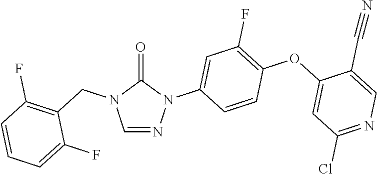 [SMILES: N#Cc1cnc(Cl)cc1Oc1ccc(-n2ncn(Cc3c(F)cccc3F)c2=O)cc1F] | yellow solid, yield: 38.4% | ¹H NMR (400 MHz, DMSO-d₆) δ 8.93 (s, 1H), 8.42 (s, 1H), 7.99 (dd, J = 12.4, 2.4 Hz, 1H), 7.86 (ddd, J = 9.2, 2.4, 1.2 Hz, 1H), 7.64 (t, J = 8.8 Hz, 1H), 7.49 (tt, J = 8.4, 6.8 Hz, 1H), 7.22-7.08 (m, 3H), 5.01 (s, 2H). | 458.0 |
| | | 6-chloro-4-(4-(4-(2,6-difluoro- | | | |
| | | benzyl)-5-oxo-4,5-dihydro-1H-1,2,4- | | | |
| | | triazol-1-yl)-2-fluorophenoxy) | | | |
| | | nicotinonitrile | | | |
| Example 396 Method 61: | +++ | 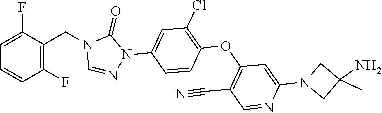 [SMILES: CC1(N)CN(c2cc(Oc3ccc(-n4ncn(Cc5c(F)cccc5F)c4=O)cc3Cl)c(C#N)cn2)C1] | yellow solid, yield: 16.1% | ¹H NMR (400 MHz, DMSO-d₆) δ 8.93 (s, 1H), 8.42 (s, 1H), 7.99 (dd, J = 12.4, 2.4 Hz, 1H), 7.86 (ddd, J = 9.2, 2.4, 1.2 Hz, 1H), 7.64 (t, J = 8.8 Hz, 1H), 7.49 (tt, J = 8.4, 6.8 Hz, 1H), 7.22-7.08 (m, 3H), 5.01 (s, 2H), 3.75 (s, 4H), 1.98 (s, 3H), 1.41 (s, 3H). | 508.1 |
| | | 6-(3-amino-3-methylazetidin-1-yl)- | | | |
| | | 4-(4-(4-(2,6-difluorobenzyl)-5- | | | |
| | | oxo-4,5-dihydro-1H-1,2,4-triazol-1- | | | |
| | | yl)-2-fluorophenoxy)nicotinonitrile | | | |
| Example 397 Method 61: | ++++ | 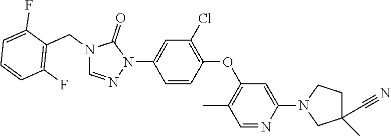 [SMILES: Cc1cnc(N2CCC(C)(C#N)C2)cc1Oc1ccc(-n2ncn(Cc3c(F)cccc3F)c2=O)cc1Cl] | white solid, yield: 16.4% | ¹H NMR (400 MHz, DMSO-d₆) δ 8.39 (s, 1H), 8.13 (d, J = 2.8 Hz, 1H), 7.97-7.86 (m, 2H), 7.55 7.43 (m, 1H), 7.35 (d, J = 9.2 Hz, 1H), 7.17 (t, J = 8.0 Hz, 2H), 5.55 (s, 1H), 5.00 (s, 2H), 3.76 (d, J = 10.8 Hz, 1H), 3.20 (d, J = 10.8 Hz, 1H), 2.36-2.31 (m, 1H), 2.12 (s, 3H), 2.01 (d, J = 12.8 Hz, 1H), | 538.2 |
| | | 1-(4-(2-chloro-4-(4-(2,6-difluoro- | | 1.43 (s, 3H), 1.17 (t, J = 7.2 Hz, | |
| | | benzyl)-5-oxo-4,5-dihydro-1H-1,2,4- | | 1H). | |
| | | triazol-1-yl)phenoxy)-5-methyl- | | | |
| | | pyridin-2-yl)-3-methylpyrrolidine- | | | |
| | | 3-carbonitrile | | | |
| Example 398 Method 61: | ++++ | 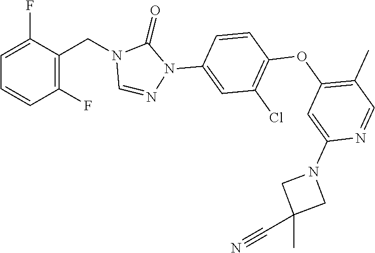 [SMILES: Cc1cnc(N2CC(C)(C#N)C2)cc1Oc1ccc(-n2ncn(Cc3c(F)cccc3F)c2=O)cc1Cl] | Off- white solid, yield 43.4% | ¹H NMR (400 MHz, Chloroform-d) δ 8.21 (d, J = 2.8 Hz, 1H), 8.00 (d, J = 8.8 Hz, 1H), 7.93 (s, 1H), 7.63 (s, 1H), 7.39 (tt, J = 8.4, 6.4 Hz, 1H), 7.17 (d, J = 9.2 Hz, 1H), 7.05- 6.95 (m, 2H), 5.32 (s, 1H), 5.00 (d, J = 1.6 Hz, 2H), 4.44-4.21 (m, 2H), 4.07-3.71 (m, 2H), 2.24 (s, 3H), 1.70 (s, 3H). | 524.2 |
| | | 1-(4-(2-chloro-4-(4-(2,6-difluoro- | | | |
| | | benzyl)-5-oxo-4,5-dihydro-1H-1,2,4- | | | |
| | | triazol-1-yl)phenoxy)-5-methyl- | | | |
| | | pyridin-2-yl)-3-methylazetidine-3- | | | |
| | | carbonitrile | | | |
| Example 399 Method 61: | +++ | 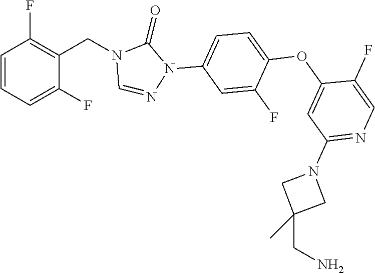 [SMILES: CC1(CN)CN(c2cc(Oc3ccc(-n4ncn(Cc5c(F)cccc5F)c4=O)cc3F)c(F)cn2)C1] | white solid, yield: 43.8% | ¹H NMR (400 MHz, CDCl₃) δ 8.47-8.25 (m, 5H), 7.96 (dd, J = 12.3, 2.5 Hz, 1H), 7.82 (ddd, J = 9.0, 2.6, 1.3 Hz, 1H), 7.66-7.42 (m, 2H), 7.24-7.05 (m, 2H), 5.78 (d, J = 6.3 Hz, 1H), 5.00 (s, 2H), 3.97 (d, J = 9.2 Hz, 2H), 3.70 (d, J = 9.1 Hz, 2H), 3.01 (q, J = 5.8 Hz, 2H), 1.31 (s, 2H). | 515.2 |
| | | 2-(4-((2-(3-(aminomethyl)-3-methyl- | | | |
| | | azetidin-1-yl)-5-fluoropyridin-4- | | | |
| | | yl)oxy)-3-fluorophenyl)-4-(2,6-di- | | | |
| | | fluorobenzyl)-2,4-dihydro-3H-1,2,4- | | | |
| | | triazol-3-one | | | |
| Example 400 Method 61: | ++++ | 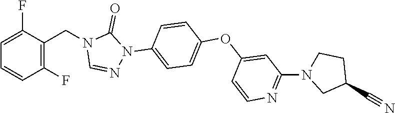 [SMILES: N#C[C@@H]1CCN(c2cc(Oc3ccc(-n4ncn(Cc5c(F)cccc5F)c4=O)cc3)ccn2)C1] | white solid, yield: 11.6% | ¹H NMR (400 MHz, DMSO-d₆) δ 8.32 (s, 1H), 7.99 (dd, J = 5.6, 1.6 Hz, 1H), 7.95-7.89 (m, 2H), 7.55- 7.43 (m, 1H), 7.27-7.11 (m, 4H), 6.19 (dt, J = 5.6, 1.6 Hz, 1H), 6.00 (d, J = 2.0 Hz, 1H), 4.99 (s, 2H), 3.71-3.64 (m, 1H), 3.63- 3.56 (m, 1H), 3.55-3.48 (m, 1H), | 475.2 |
| | | (R)-1-(4-(4-(4-(2,6-difluorobenzyl)- | | 3.48-3.39 (m, 2H), 2.38-2.28 | |
| | | 5-oxo-4,5-dihydro-1H-1,2,4-triazol- | | (m, 1H), 2.27-2.16 (m, 1H). | |
| | | 1-yl)phenoxy)pyridin-2-yl)pyrrolidine- | | | |
| | | 3-carbonitrile | | | |
| Example 401 Method 61 | ++++ | 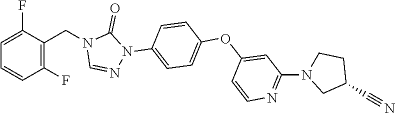 [SMILES: N#C[C@H]1CCN(c2cc(Oc3ccc(-n4ncn(Cc5c(F)cccc5F)c4=O)cc3)ccn2)C1] | white solid, yield: 12% | ¹H NMR (400 MHz, DMSO-d₆) δ 8.32 (s, 1H), 7.99 (d, J = 5.6 Hz, 1H), 7.97-7.87 (m, 2H), 7.48 (tt, J = 8.4, 6.4 Hz, 1H), 7.32-7.11 (m, 4H), 6.19 (dd, J = 5.6, 2.0 Hz, 1H), 5.99 (d, J = 2.0 Hz, 1H), 4.98 (s, 2H), 3.74-3.63 (m, 1H), 3.62- 3.57 (m, 1H), 3.55-3.48 (m, 1H), | 475.2 |
| | | (S)-1-(4-(4-(4-(2,6-difluorobenzyl)- | | 3.46-3.37 (m, 2H), 2.31 (dq, J = | |
| | | 5-oxo-4,5-dihydro-1H-1,2,4- | | 13.6, 6.8, 6.0 Hz, 1H), 2.26-2.15 | |
| | | triazol-1-yl)phenoxy)pyridin-2-yl) | | (m, 1H). | |
| | | pyrrolidine-3-carbonitrile | | | |

### Example 402: Method 62

### 4-(4-(4-(2,6-difluorobenzyl)-5-oxo-4,5-dihydro-1H-1,2,4-triazol-1-yl)-2-fluorophenoxy)-5-fluoropicolinonitrile

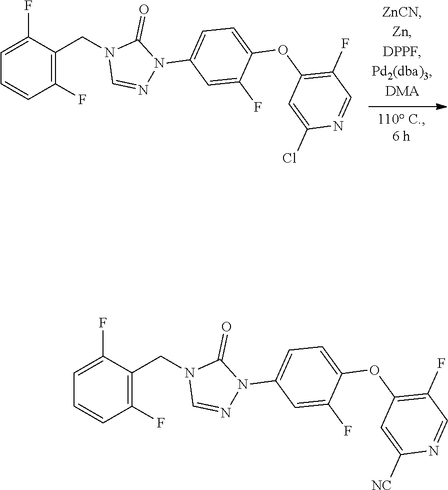 [SMILES: O=c1n(Cc2c(F)cccc2F)cnn1-c1ccc(Oc2cc(Cl)ncc2F)c(F)c1 ; N#Cc1cc(Oc2ccc(-n3ncn(Cc4c(F)cccc4F)c3=O)cc2F)c(F)cn1]

A mixture of 2-{4-[(2-chloro-5-fluoropyridin-4-yl)oxy]-3-fluorophenyl}-4-[(2,6-difluorophenyl)methyl]-1,2,4-triazol-3-one (100 mg, 0.22 mmol), ZnCN (52.09 mg,0.44 mmol), Zn(14.51 mg, 0.22 mmol), DPPF(61.48 mg, 0.11 mmol) and Pd₂(dba)₃(101.55 mg,0.11 mmol) in DMA (10 mL). The reaction mixture was stirred at 110° C. for 6 h. Water (20 mL) was added to the mixture, and the mixture was extracted with EtOAc (30 mL×3). The combined organic layers were washed with brine (4 mL), dried over anhydrous Na₂SO₄, and after filtration, the filtrate was concentrated under reduced pressure. The residue was purified by flash with the following condition (PE:EtOAc=1:1) to afford the titled compound (8.3 mg, 8.39% yield) as an off-white solid. MS (m/z): 441.9 [M+H]⁺. ¹HNMR (400 MHz, CDCl₃) δ 8.53 (d, J 2.5 Hz, 1H), 8.04 (dd, J 12.0, 2.5 Hz, 1H), 7.94 (ddd, J=9.0, 2.5, 1.5 Hz, 1H), 7.63 (s, 1H), 7.37 (tt, J=8.4, 6.5 Hz, 1H), 7.27 (d, J=8.7 Hz, 1H), 7.05 (dd, J=6.3, 1.3 Hz, 1H), 7.02-6.96 (m, 2H), 4.99 (s, 2H).

The synthesis procedure of Examples (Compounds) 403-406 were similar to the procedure of Example 402.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 402 Method 62 | ++++ | 4-(4-(4-(2,6-difluorobenzyl)-5-oxo-4,5-dihydro-1H-1,2,4- triazol-1-yl)-2-fluorophenoxy)-5-fluoropicolinonitrile | white solid, yield: 8.39% | ¹H NMR (400 MHz, DMSO-d₆) δ 8.53 (d, J = 2.5 Hz, 1H), 8.04 (dd, J = 12.0, 2.5 Hz, 1H), 7.94 (ddd, J = 9.0, 2.5, 1.5 Hz, 1H), 7.63 (s, 1H), 7.37 (tt, J = 8.4, 6.5 Hz, 1H), 7.27 (d, J = 8.7 Hz, 1H), 7.05 (dd, J = 6.3, 1.3 Hz, 1H), 7.02-6.96 (m, 2H), 4.99 (s, 2H). | 441.9 |
| Example 403 Method 62 | +++ | 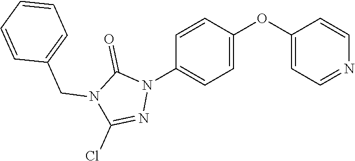 [SMILES: O=c1n(-c2ccc(Oc3ccncc3)cc2)nc(Cl)n1Cc1ccccc1] 4-benzyl-5-chloro-2-(4-(pyridin-4-yloxy)phenyl)-2,4- dihydro-3H-1,2,4-triazol-3-one | white solid, yield: 15.9% | ¹H NMR (400 MHz, MeOD) δ 8.11-8.01 (m, 2H), 7.65-7.53 (m, 7H), 7.44-7.36 (m, 4H), 4.90 (s, 2H). | 379.8 |
| Example 404 Method 62 | +++ | 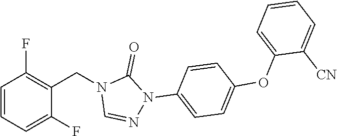 [SMILES: N#Cc1ccccc1Oc1ccc(-n2ncn(Cc3c(F)cccc3F)c2=O)cc1] 2-(4-(4-(2,6-difluorobenzyl)-5-oxo-4,5-dihydro-1H-1,2,4- triazol-1-yl)phenoxy)benzonitrile | white solid, yield: 48.6% | ¹HNMR (400 MHz, CDCl₃) δ 8.06-7.99 (m, 2H), 7.70-7.57 (m, 2H), 7.45-7.34 (m, 2H), 7.19-7.08 (m, 3H), 6.99 (dd, 2H), 6.83 (d, J = 8.6 Hz, 1H), 4.98 (s, 2H). | 405.4 |
| Example 405 Method 62 | +++ | 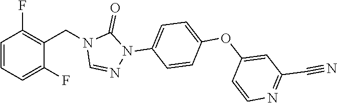 [SMILES: N#Cc1cc(Oc2ccc(-n3ncn(Cc4c(F)cccc4F)c3=O)cc2)ccn1] 4-(4-(4-(2,6-difluorobenzyl)-5-oxo-4,5-dihydro-1H-1,2,4- triazol-1-yl)phenoxy)picolinonitrile | white solid, yield: 22.3% | ¹NMR (400 MHz, CDCl₃) δ 8.56 (d, J = 5.8 Hz, 1H), 8.31 (s, 1H), 7.98-7.92 (m, 2H), 7.68 (d, J = 2.4 Hz, 1H), 7.45 (s, 1H), 7.33-7.28 (m, 2H), 7.18 (dd, J = 5.8, 2.6 Hz, 1H), 7.13 (t, J = 8.1 Hz, 2H), 4.96 (s, 2H). | 406.1 |
| Example 406 Method 62 | ++++ | 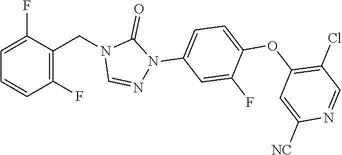 [SMILES: N#Cc1cc(Oc2ccc(-n3ncn(Cc4c(F)cccc4F)c3=O)cc2F)c(Cl)cn1] 5-chloro-4-(4-(4-(2,6-difluorobenzyl)-5-oxo-4,5-dihydro- 1H-1,2,4-triazol-1-yl)-2-fluorophenoxy)picolinonitrile | off-white solid, yield: 1.4% | NMR (400 MHz, DMSO-d₆) δ 8.64 (d, J = 3.2 Hz, 1H), 8.05 (dd, J = 11.9, 2.5 Hz, 1H), 7.96 (ddd, J = 9.0, 2.5, 1.5 Hz, 1H), 7.63 (s, 1H), 7.40-7.35 (m, 1H), 7.29-7.25 (m, 1H), 7.02-6.96 (m, 2H), 6.92 (d, J = 1.3 Hz, 1H), 4.99 (s, 2H). | 457.9 |

### Example 407: Method 63

### 4-[(2,6-difluorophenyl)methyl]-2-[4-({2-[methyl(oxan-4-ylmethyl)amino]pyridin-4-yl}oxy)phenyl]-1,2,4-triazol-3-one

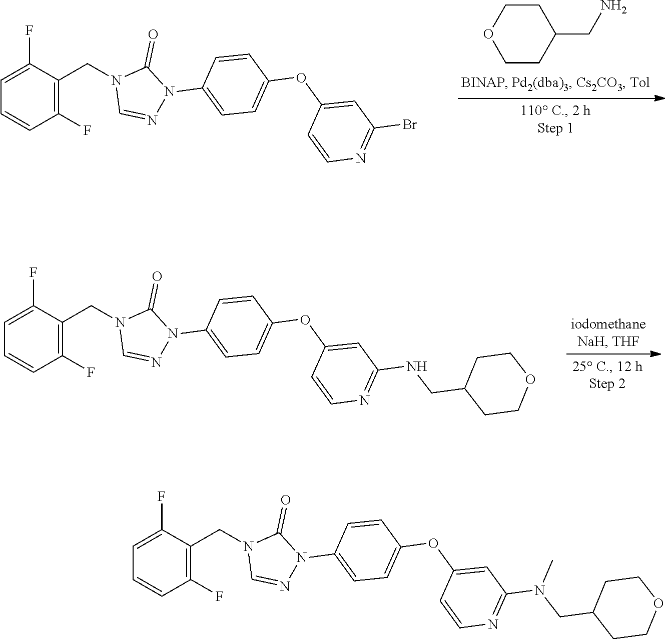 [SMILES: O=c1n(Cc2c(F)cccc2F)cnn1-c1ccc(Oc2ccnc(Br)c2)cc1 ; NCC1CCOCC1 ; O=c1n(Cc2c(F)cccc2F)cnn1-c1ccc(Oc2ccnc(NCC3CCOCC3)c2)cc1 ; CN(CC1CCOCC1)c1cc(Oc2ccc(-n3ncn(Cc4c(F)cccc4F)c3=O)cc2)ccn1]

### Step 1: 4-[(2,6-difluorophenyl)methyl]-2-[4-({2-[(oxan-4-ylmethyl)amino]pyridin-4-yl}oxy)phenyl]-1,2,4-triazol-3-one

A mixture of 2-{4-[(2-bromopyridin-4-yl)oxy]phenyl}-4-[(2,6-difluorophenyl)methyl]-1,2,4-triazol-3-one (300 mg, 0.6532 mmol), oxan-4-ylmethanamine (75.23 mg, 0.6532 mmol), Pd₂(dba)₃ (299.07 mg, 0.3266 mmol), CS₂CO₃ (851.30 mg, 2.6128 mmol) and Binap (325.28 mg, 0.5225 mmol) in toluene (15 mL) was stirred at 110° C. for 2 h. Water (20 mL) was added to the mixture, and the mixture was extracted with EtOAc (30 mL×3). The combined organic layers were washed with brine (4 mL), dried over anhydrous Na₂SO₄, and after filtration, the filtrate was concentrated under reduced pressure. The residue was purified by silica gel column chromatography, eluted with (PE:EtOAc) (1:1) to afford 4-[(2,6-difluorophenyl)methyl]-2-[4-({2-[(oxan-4-ylmethyl)amino]pyridin-4-yl}oxy)phenyl]-1,2,4-triazol-3-one (30 mg, 9.32% yield) as a white solid. MS (m/z): 494.0[M+H]⁺.

### Step 2: 4-[(2,6-difluorophenyl)methyl]-2-[4-({2-[methyl(oxan-4-ylmethyl)amino]pyridin-4-yl}oxy)phenyl]-1,2,4-triazol-3-one

A mixture of 4-[(2,6-difluorophenyl)methyl]-2-[4-({2-[(oxan-4-ylmethyl)amino]pyridin-4-yl}oxy)phenyl]-1,2,4-triazol-3-one(30 mg, 0.0608 mmol), iodomethane(8.63 mg,0.0608 mmol), and NaH (2.43 mg, 0.0608 mmol) in THF(5 mL) was stirred at 25° C. for 12 h. Water (20 mL) was added to the mixture, and the mixture was extracted with EtOAc (30 mL×3). The combined organic layers were washed with brine (4 mL), dried over anhydrous Na₂SO₄, and after filtration, the filtrate was concentrated under reduced pressure. The residue was purified by silica gel column chromatography, eluted with (PE:EtOAc=1:1) to afford (5 mg, 14.64% yield) as a white solid. MS (m/z): 508.0 [M+H]⁺. ¹H NMR (400 MHz,) δ 8.00 (dd, J=13.4, 7.3 Hz, 3H), 7.58 (s, 1H), 7.42-7.31 (m, 1H), 7.16-7.09 (m, 2H), 6.98 (dd, J=8.3, 7.5 Hz, 2H), 6.23-6.01 (m, 1H), 5.97 (d, J=2.1 Hz, 1H), 4.98 (s, 2H), 3.94 (dd, J=11.3, 3.0 Hz, 2H), 3.35 (ddd, J=14.5, 13.5, 4.7 Hz, 4H), 3.00 (s, 3H), 2.00 (s, 1H), 1.39-1.26 (m, 3H), 0.95-0.75 (m, 1H).

The synthesis procedure of Example (Compound) 408 was similar to the procedure of Example 407.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 407 Method 63 | +++ | 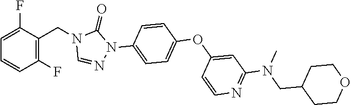 [SMILES: CN(CC1CCOCC1)c1cc(Oc2ccc(-n3ncn(Cc4c(F)cccc4F)c3=O)cc2)ccn1] 4-(2,6-difluorobenzyl)-2-(4-((2-(methyl((tetrahydro-2H-pyran- 4-yl)methyl)amino)pyridin-4-yl)oxy)phenyl)-2,4-dihydro-3H- 1,2,4-triazol-3-one | off-white solid, yield: 1.4% | NMR (400 MHz, DMSO-d₆) δ 8.00 (dd, J = 13.4, 7.3 Hz, 3H), 7.58 (s, 1H), 7.42-7.31 (m, 1H), 7.16-7.09 (m, 2H), 6.98 (dd, J = 8.3, 7.5 Hz, 2H), 6.23-6.01 (m, 1H), 5.97 (d, J = 2.1 Hz, 1H), 4.98 (s, 2H), 3.94 (dd, J = 11.3, 3.0 Hz, 2H), 3.35 (ddd, J = 14.5, 13.5, 4.7 Hz, 4H), 3.00 (s, 3H), 2.00 (s, 1H), 1.39-1.26 (m, 3H), 0.95-0.75 (m, 1H). | 508.0 |
| Example 408 Method 63 | ++++ | 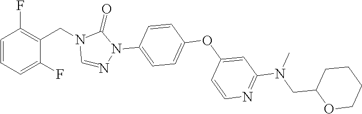 [SMILES: CN(CC1CCCCO1)c1cc(Oc2ccc(-n3ncn(Cc4c(F)cccc4F)c3=O)cc2)ccn1] 4-(2,6-difluorobenzyl)-2-(4-((2-(methyl((tetrahydro-2H-pyran- 2-yl)methyl)amino)pyridin-4-yl)oxy)phenyl)-2,4-dihydro-3H- 1,2,4-triazol-3-one | white solid, yield: 20.2% | ¹H NMR (400 MHz, DMSO-d₆) δ 8.09 (d, J = 9.0 Hz, 2H), 7.62 (s, 2H), 7.41-7.33 (m, 1H), 7.15 (s, 2H), 7.03-6.95 (m, 2H), 6.37 (dd, J = 9.6, 3.4 Hz, 1H), 6.18 (t, J = 4.0 Hz, 1H), 4.99 (s, 2H), 3.92-3.84 (m, 1H), 3.52-3.31 (m, 2H), 3.12 (ddd, J = 17.6, 15.8, 14.1 Hz, 2H), 1.95 (s, 3H), 1.82 (s, 1H), 1.52- 1.40 (m, 3H), 1.33-1.22 (m, 2H). | 507.9 |

### Example 409: Method 64

### 2-(4-((3-amino-2-(3-ethoxyazetidin-1-yl)pyridin-4-yl)oxy)phenyl)-4-(2,6-difluorobenzyl)-2,4-dihydro-3H-1,2,4-triazol-3-one

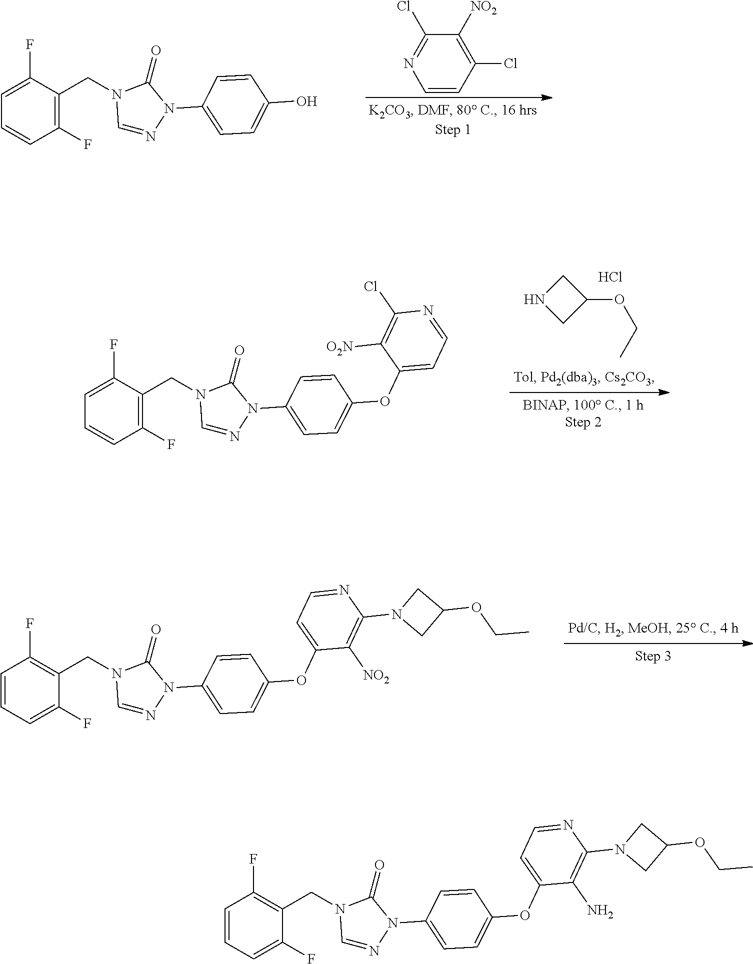

### Step 1: 2-{4-[(2-chloro-3-nitropyridin-4-yl)oxy]phenyl}-4-[(2,6-difluorophenyl)methyl]-1,2,4-triazol-3-one

A solution of 4-[(2,6-difluorophenyl)methyl]-2-(4-hydroxyphenyl)-1,2,4-triazol-3-one (300 mg, 0.99 mmol), 2,4-dichloro-3-nitropyridine (191 mg, 0.99 mmol) and K₂CO₃ (410 mg, 2.96 mmol) in DMF was stirred under nitrogen at 80° C. for 16 h. The mixture was allowed to cool down to rt. The resulted mixture was diluted with water (100 mL). The resulted mixture was extracted with EtOAc (3×80 mL). The combined Organic layers were washed with brine (100 mL), dried over anhydrous Na₂SO₄, and after filtration, the filtrate was concentrated under reduced pressure. The residue was purified by silica gel column chromatography, eluted with (PE:EtOAc) (3:7) to afford 2-{4-[(2-chloro-3-nitropyridin-4-yl)oxy]phenyl}-4-[(2,6-difluorophenyl)methyl]-1,2,4-triazol-3-one (400 mg, 88% yield) as a yellow solid. MS (m/z): 460.0 [M+H]⁺.

### Step 2: 4-[(2,6-difluorophenyl)methyl]-2-(4-{[2-(3-ethoxyazetidin-1-yl)-3-nitropyridin-4-yl]oxy}phenyl)-1,2,4-triazol-3-one

A mixture of 2-{4-[(2-chloro-3-nitropyridin-4-yl)oxy]phenyl}-4-[(2,6-difluorophenyl)methyl]-1,2,4-triazol-3-one (120 mg, 0.26 mmol), 3-ethoxyazetidine hydrochloride (39.5 mg, 0.288 mmol), tris(dibenzylideneacetone)dipalladium (47.8 mg, 0.05 mmol), Cs₂CO₃ (425 mg, 1.3 mmol) and 2,2′-bis(diphenylphosphino)-1,1′-binaphthyl(32.5 mg, 0.05 mmol) in 5 mL of Tol was degassed and purged with N₂, and then stirred at 110° C. for 1 h. The reaction mixture was concentrated under vacuum. The residue was purified by chromatography (15% EA-DCM) to afford the 4-[(2,6-difluorophenyl)methyl]-2-(4-{[2-(3-ethoxyazetidin-1-yl)-3-nitropyridin-4-yl]oxy}phenyl)-1,2,4-triazol-3-one as a white solid. (95 mg, 69% yield). LC-MS (m/z): 525.2 [M+H]⁺.

### Step 3: 2-(4-((3-amino-2-(3-ethoxyazetidin-1-yl)pyridin-4-yl)oxy)phenyl)-4-(2,6-difluorobenzyl)-2,4-dihydro-3H-1,2,4-triazol-3-one

A solution of 4-[(2,6-difluorophenyl)methyl]-2-(4-{[2-(3-ethoxyazetidin-1-yl)-3-nitropyridin-4-yl]oxy}phenyl)-1,2,4-triazol-3-one (60 mg, 0.11 mmol), Pd/C(30 mg) in MeOH(5 mL) was degassed and purged under H₂, and then stirred at 25° C. for 2 h. The mixture was filtered and the filtrate was concentrated under reduced pressure. The residue was purified by silica gel column chromatography, eluted with (MeOH:DCM=1:20) to afford 2-(4-{[3-amino-2-(3-ethoxyazetidin-1-yl)pyridin-4-yl]oxy}phenyl)-4-[(2,6-difluorophenyl)met hyl]-1,2,4-triazol-3-one (12 mg, 43% yield) as a white solid. MS (m/z): 495.2 [M+H]⁺. ¹H NMR (400 MHz, CDCl₃) δ 7.94 (d, J=9.1 Hz, 2H), 7.61 (d, J=5.7 Hz, 1H), 7.57 (s, 1H), 7.35 (tt, J=8.4, 6.5 Hz, 1H), 7.11-7.04 (m, 2H), 7.02-6.92 (m, 2H), 6.25 (d, J=5.7 Hz, 1H), 4.97 (s, 3H), 4.37 (m, 1H), 4.31 (m, 1H), 4.02 (q, 8.0 Hz, 2H), 3.51-3.47 (m, 2H), 1.23 (t, J=7.0 Hz, 3H).

The synthesis procedure of Example (Compound) 410 was similar to the procedure of Example 409.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 409 Method 64 | +++ | 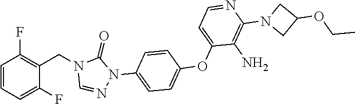 [SMILES: CCOC1CN(c2nccc(Oc3ccc(-n4ncn(Cc5c(F)cccc5F)c4=O)cc3)c2N)C1] 2-(4-((3-amino-2-(3-ethoxyazetidin-1-yl)pyridin-4-yl)oxy) phenyl)-4-(2,6-difluorobenzyl)-2,4-dihydro-3H-1,2,4-triazol- 3-one | white solid, yield: 43% | ¹H NMR (400 MHZ, CDCl₃) δ 7.94 (d, J = 9.1 Hz, 2H), 7.61 (d, J = 5.7 Hz, 1H), 7.57 (s, 1H), 7.35 (tt, J = 8.4, 6.5 Hz, 1H), 7.11-7.04 (m, 2H), 7.02- 6.92 (m, 2H), 6.25 (d, J = 5.7 Hz, 1H), 4.97 (s, 3H), 4.37 (m, 1H), 4.31 (m, 1H), 4.02 (q, 8.0 Hz, 2H), 3.51-3.47 (m, 2H), 1.23 (t, J = 7.0 Hz, 3H). | 495.2 |
| Example 410 Method 64 | +++ | 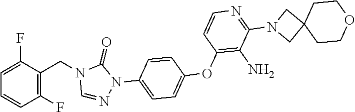 [SMILES: Nc1c(Oc2ccc(-n3ncn(Cc4c(F)cccc4F)c3=O)cc2)ccnc1N1CC2(CCOCC2)C1] 2-(4-((3-amino-2-(7-oxa-2-azaspiro[3.5]nonan-2-yl)pyridin-4- yl)oxy)phenyl)-4-(2,6-difluorobenzyl)-2,4-dihydro-3H-1,2,4- triazol-3-one | white solid, yield: 38% | ¹H NMR (400 MHz, CDCl₃) δ 7.98-7.90 (m, 2H), 7.61 (d, J = 5.8 Hz, 1H), 7.57 (s, 1H), 7.36 (tt, J = 8.4, 6.6 Hz, 1H), 7.12- 7.04 (m, 2H), 6.97 (dd, J = 8.3, 7.6 Hz, 2H), 6.24 (d, J = 5.8 Hz, 1H), 4.97 (s, 2H), 3.93 (s, 4H), 3.70-3.63 (t, 4H), 3.52 (s, 2H), 1.88-1.83 (t, 4H). | 521.1 |

### Example 411: Method 65

### 2-(4-((5-chloro-2-(1,6-diazaspiro[3.3]heptan-6-yl)pyridin-4-yl)oxy)-3-fluorophenyl)-4-(2,6-difluorobenzyl)-2,4-dihydro-3H-1,2,4-triazol-3-one hydrochloride

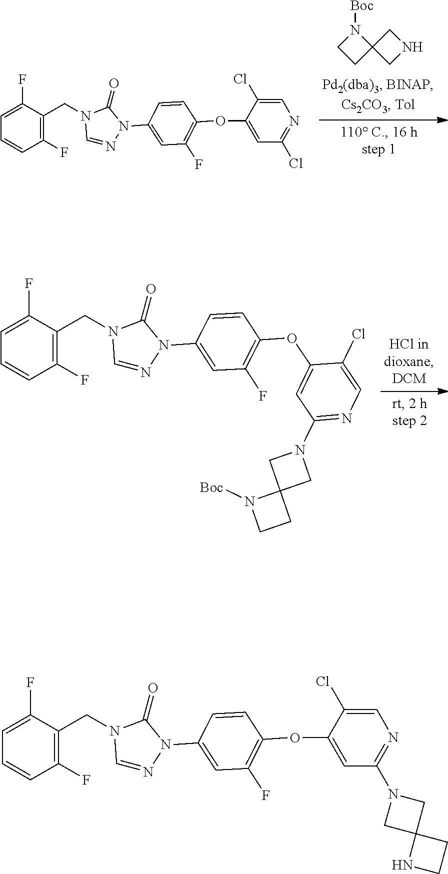 [SMILES: CC(C)(C)OC(=O)N1CCC12CNC2 ; O=c1n(Cc2c(F)cccc2F)cnn1-c1ccc(Oc2cc(Cl)ncc2Cl)c(F)c1 ; CC(C)(C)OC(=O)N1CCC12CN(c1cc(Oc3ccc(-n4ncn(Cc5c(F)cccc5F)c4=O)cc3F)c(Cl)cn1)C2 ; O=c1n(Cc2c(F)cccc2F)cnn1-c1ccc(Oc2cc(N3CC4(CCN4)C3)ncc2Cl)c(F)c1]

### Step 1: tert-butyl 6-(5-chloro-4-(4-(4-(2,6-difluorobenzyl)-5-oxo-4,5-dihydro-1H-1,2,4-triazol-1-yl)-2-fluorophenoxy)pyridin-2-yl)-1,6-diazaspiro[3.3]heptane-1-carboxylate

A solution of 2-{4-[(2,5-dichloropyridin-4-yl)oxy]-3-fluorophenyl}-4-[(2,6-difluorophenyl) methyl]-1,2,4-triazol-3-one (80 mg, 0.17 mmol), tert-butyl {1,6-diazaspiro[3.3]heptan-1-yl}formate (41 mg, 0.2 mmol), Pd2(dba)3 (47 mg, 0.5 mmol), BINAP (32 mg, 0.05 mmol), Cs₂CO₃ (112 mg, 0.34 mmol) in toluene (5 mL) was stirred at 110° C. for 16 h. The resulted mixture was diluted with water (30 mL). The resulted mixture was extracted with EtOAc (3×20 mL). The combined organic layers were washed with brine (30 mL), dried over anhydrous Na₂SO₄, and after filtration, the filtrate was concentrated under reduced pressure. The residue was purified by Prep-TLC (EtOAc: PE=1:1) to afford the titled compound (70.0 mg, 58.4% yield) as a white solid. MS (m/z): 629.3 [M+H]⁺. ¹H NMR (400 MHz, CDCl₃) δ 8.08 (s, 1H), 7.98 (dd, J=11.9, 2.2 Hz, 1H), 7.86 (d, J=8.5 Hz, 1H), 7.63 (s, 1H), 7.39 (tt, J=8.4, 6.5 Hz, 1H), 7.21 (t, J=8.7 Hz, 1H), 7.04-6.96 (m, 2H), 5.45 (d, J=0.8 Hz, 1H), 5.00 (s, 2H), 4.56 (s, OH), 4.34 (d, J=7.8 Hz, 1H), 3.97 (d, J=7.9 Hz, 2H), 3.78 (t, J=7.2 Hz, 2H), 2.41 (t, J=7.2 Hz, 2H), 1.34 (d, J=51.6 Hz, 9H).

### Step 2: 2-(4-((5-chloro-2-(1,6-diazaspiro[3.3]heptan-6-yl)pyridin-4-yl)oxy)-3-fluorophenyl)-4-(2,6-difluorobenzyl)-2,4-dihydro-3H-1,2,4-triazol-3-one hydrochloride

A solution of tert-butyl 6-(5-chloro-4-(4-(4-(2,6-difluorobenzyl)-5-oxo-4,5-dihydro-1H-1,2,4-triazol-1-yl)-2-fluorophenoxy)pyridin-2-yl)-1,6-diazaspiro[3.3]heptane-1-carboxylate (60 mg, 0.1 mmol), and HCl (1 mL, 4M in dioxane) in DCM (2 mL) was stirred at rt for 2 h. The resulted mixture was concentrated under reduced pressure to afford the titled compound 411(25.1 mg, 45.7% yield) as a white solid. MS (m/z): 529.3 [M+H]⁺. ¹H NMR (400 MHz, CDCl₃) δ 10.69 (s, 1H), 8.04 (d, J=45.1 Hz, 3H), 7.65 (s, 1H), 7.38 (s, 2H), 6.99 (s, 3H), 5.58 (s, 2H), 5.00 (s, 3H), 4.57 (s, 1H), 4.06 (s, 2H), 2.92 (d, J=29.6 Hz, 2H).

The synthesis procedure of Examples (Compounds) 412-424 was similar to the procedure of Example 411.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 411 Method 65 | ++++ | 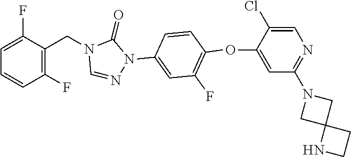 [SMILES: O=c1n(Cc2c(F)cccc2F)cnn1-c1ccc(Oc2cc(N3CC4(CCN4)C3)ncc2Cl)c(F)c1] 2-(4-((5-chloro-2-(1,6-diazaspiro[3.3]heptan-6-yl)pyridin- 4-yl)oxy)-3-fluorophenyl)-4-(2,6-difluorobenzyl)- 2,4-dihydro-3H-1,2,4-triazol-3-one hydrochloride | white solid, yield: 45.7% | ¹H NMR (400 MHz, CDCl₃) δ 10.69 (s, 1H), 8.04 (d, J = 45.1 Hz, 3H), 7.65 (s, 1H), 7.38 (s, 2H), 6.99 (s, 3H), 5.58 (s, 2H), 5.00 (s, 3H), 4.57 (s, 1H), 4.06 (s, 2H), 2.92 (d, J = 29.6 Hz, 2H). | 529.3 |
| Example 412 Method 65 | +++ | 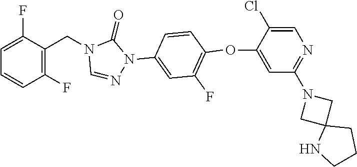 [SMILES: O=c1n(Cc2c(F)cccc2F)cnn1-c1ccc(Oc2cc(N3CC4(CCCN4)C3)ncc2Cl)c(F)c1] 2-(4-((5-chloro-2-(2,5-diazaspiro[3.4]octan-2-yl)pyridin-4-yl) oxy)-3-fluorophenyl)-4-(2,6-difluorobenzyl)-2,4-dihydro-3H- 1,2,4-triazol-3-one | white solid, yield: 35% | ¹H NMR (400 MHz, DMSO- d₆) δ 9.38 (s, 2H), 8.39 (s, 1H), 8.18 (s, 1H), 7.94 (dd, J = 12.3, 2.5 Hz, 1H), 7.80 (d, J = 7.9 Hz, 1H), 7.47 (q, J = 8.7 Hz, 2H), 7.16 (t, J = 8.1 Hz, 2H), 5.68 (s, 1H), 5.00 (s, 2H), 4.11 (d, J = 9.5 Hz, 2H), 3.94 (d, J = 9.4 Hz, 2H), 3.20-3.16 (m, 2H), 2.11 (t, J = 7.2 Hz, 2H), 1.95-1.81 (m, 2H). | 543.2 |
| Example 413 Method 65 | ++++ | 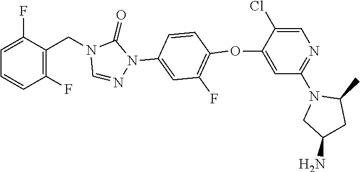 [SMILES: C[C@H]1C[C@@H](N)CN1c1cc(Oc2ccc(-n3ncn(Cc4c(F)cccc4F)c3=O)cc2F)c(Cl)cn1] 2-(4-((2-((2S,4R)-4-amino-2-methylpyrrolidin-1-yl)-5- chloropyridin-4-yl)oxy)-3-fluorophenyl)-4-(2,6- difluorobenzyl)-2,4-dihydro-3H-1,2,4-triazol-3-one | white solid, yield: 68.5% | ¹H NMR (400 MHz, DMSO- d₆) δ 8.06 (s, 2H), 7.96 (d, J = 12.1 Hz, 1H), 7.86 (d, J = 8.9 Hz, 1H), 7.50-7.42 (m, 1H), 7.35 (t, J = 8.7 Hz, 1H), 7.07 (t, J = 8.0 Hz, 2H), 5.72 (s, 1H), 5.05 (s, 2H), 3.99-3.87 (m, 1H), 3.87-3.78 (m, 1H), 3.76-3.62 (m, 1H), 2.69-2.56 (m, 1H), 1.75-1.66 (m, 2H), 1.22 (d, J = 6.0 Hz, 3H). | 531.3 |
| Example 414 Method 65 | + | 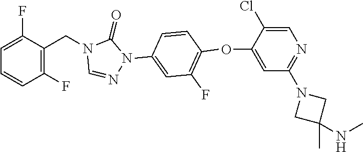 [SMILES: CNC1(C)CN(c2cc(Oc3ccc(-n4ncn(Cc5c(F)cccc5F)c4=O)cc3F)c(Cl)cn2)C1] 2-(4-((5-chloro-2-(3-methyl-3-(methylamino)azetidin-1-yl) pyridin-4-yl)oxy)-3-fluorophenyl)-4-(2,6-difluorobenzyl)-2,4- dihydro-3H-1,2,4-triazol-3-one | white solid, yield: 70% | ¹H NMR (400 MHZ, MeOD) δ 8.36 (s, 1H), 8.03-7.93 (m, 2H), 7.87 (s, 1H), 7.43 (s, 1H), 7.40-7.30 (m, 1H), 6.97 (t, J = 8.0 Hz, 2H), 6.11 (s, 1H), 5.04 (s, 2H), 3.67-3.44 (m, 4H), 2.98 (s, 3H), 1.51 (s, 3H). | 531.1 |
| Example 415 Method 65 | ++++ | 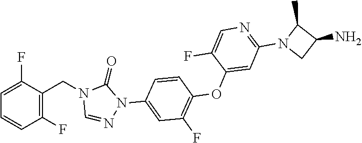 [SMILES: C[C@H]1[C@@H](N)CN1c1cc(Oc2ccc(-n3ncn(Cc4c(F)cccc4F)c3=O)cc2F)c(F)cn1] 2-(4-((2-((2S,3S)-3-amino-2-methylazetidin-1-yl)-5- fluoropyridin-4-yl)oxy)-3-fluorophenyl)-4-(2,6- difluorobenzyl)-2,4-dihydro-3H-1,2,4-triazol-3-one | white solid, yield: 79.8% | ¹H NMR (400 MHz, DMSO- d₆) δ 8.51-8.32 (m, 4H), 8.19 (d, J = 3.0 Hz, 1H), 7.94 (dd, J = 12.5, 2.5 Hz, 1H), 7.84- 7.72 (m, 1H), 7.53-7.41 (m, 2H), 7.24-7.10 (m, 2H), 5.90 (d, J = 5.7 Hz, 1H), 5.00 (s, 2H), 4.45 (p, J = 6.6 Hz, 1H), 4.05-3.87 (m, 2H), 3.75 (dd, J = 9.2, 2.9 Hz, 1H), 1.34 (d, J = 6.6 Hz, 3H). | 501.1 |
| Example 416 Method 65 | ++++ | 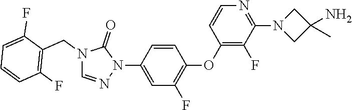 [SMILES: CC1(N)CN(c2nccc(Oc3ccc(-n4ncn(Cc5c(F)cccc5F)c4=O)cc3F)c2F)C1] 2-(4-((2-(3-amino-3-methylazetidin-1-yl)-3-fluoropyridin- 4-yl)oxy)-3-fluorophenyl)-4-(2,6-difluorobenzyl)-2,4-dihydro- 3H-1,2,4-triazol-3-one | yellow solid, yield: 12% | ¹H NMR (400 MHZ, CD₃OD) δ 8.46 (s, 1H), 8.05 (s, 1H), 7.93 (dd, J = 12.3, 2.4 Hz, 1H), 7.84 (d, J = 9.0 Hz, 1H), 7.75 (d, J = 5.8 Hz, 1H), 7.51-7.40 (m, 1H), 7.33 (t, J = 8.8 Hz, 1H), 7.07 (t, J = 8.1 Hz, 1H), 6.26 (t, J = 5.7 Hz, 1H), 5.05 (s, 2H), 4.27-4.06 (m, 4H), 1.66 (s, 3H). | 501.2 |
| Example 417 Method 65 | ++++ | 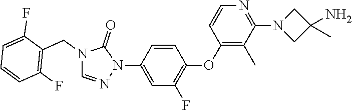 [SMILES: Cc1c(Oc2ccc(-n3ncn(Cc4c(F)cccc4F)c3=O)cc2F)ccnc1N1CC(C)(N)C1] 2-(4-((2-(3-amino-3-methylazetidin-1-yl)-3-methylpyridin- 4-yl)oxy)-3-fluorophenyl)-4-(2,6-difluorobenzyl)-2,4-dihydro- 3H-1,2,4-triazol-3-one | yellow solid, yield: 23% | ¹H NMR (400 MH_{Z}, CD₃OD) δ 8.04 (s, 1H), 7.89 (dd, J = 12.2, 2.4 Hz, 1H), 7.83-7.75 (m, 2H), 7.50-7.40 (m, 1H), 7.23 (t, J = 8.8 Hz, 1H), 7.06 (t, J = 8.1 Hz, 2H), 6.12 (d, J = 5.8 Hz, 1H), 5.05 (s, 2H), 4.04 (d, J = 2.1 Hz, 4H), 2.21 (s, 3H), 1.58 (s, 3H). | 497.2 |
| Example 418 Method 65 | ++++ | 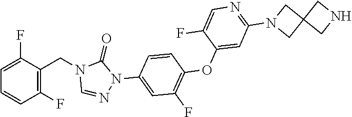 [SMILES: O=c1n(Cc2c(F)cccc2F)cnn1-c1ccc(Oc2cc(N3CC4(CNC4)C3)ncc2F)c(F)c1] 4-(2,6-difluorobenzyl)-2-(3-fluoro-4-((5-fluoro-2-(2,6- diazaspiro[3.3]heptan-2-yl)pyridin-4-yl)oxy)phenyl)-2,4- dihydro-3H-1,2,4-triazol-3-one | | ¹H NMR (400 MHz, DMSO- d₆) δ 8.40 (s, 1H), 8.11 (d, J = 3.0 Hz, 1H), 7.93 (dd, J = 12.4, 2.5 Hz, 1H), 7.79-7.75 (m, 1H), 7.53-7.42 (m, 2H), 7.17 (t, J = 8.1 Hz, 2H), 5.79-5.68 (m, 1H), 5.00 (s, 2H), 3.86 (s, 4H), 3.52 (s, 2H), 3.17 (d, J = 5.1 Hz, 2H). | 513.5 |
| Example 419 Method 65 | +++ | 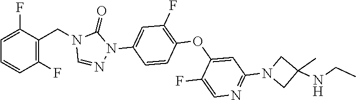 [SMILES: CCNC1(C)CN(c2cc(Oc3ccc(-n4ncn(Cc5c(F)cccc5F)c4=O)cc3F)c(F)cn2)C1] 4-(2,6-difluorobenzyl)-2-(4-((2-(3-(ethylamino)-3- methylazetidin-1-yl)-5-fluoropyridin-4-yl)oxy)-3- fluorophenyl)-2,4-dihydro-3H-1,2,4-triazol-3-one | | ¹H NMR (400 MHz, DMSO- d₆) δ 8.41 (s, 1H), 8.16 (d, J = 2.8 Hz, 1H), 7.94 (dd, J = 12.4, 2.8 Hz, 1H), 7.82-7.77 (m, 1H), 7.53-7.45 (m, 2H), 7.22-7.13 (m, 2H), 5.79 (d, J = 5.6 Hz, 1H), 5.00 (s, 2H), 3.91 (d, J = 8.4 Hz, 2H), 3.69 (d, J = 8.8 Hz, 2H), 2.81 (q, J = 6.8 Hz, 2H), 1.49 (s, 3H), 1.13 (t, J = 6.8 Hz, 3H). | 529.2 |
| Example 420 Method 65 | ++++ | 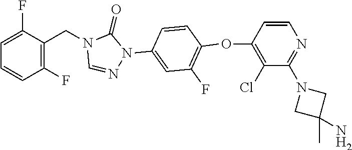 [SMILES: CC1(N)CN(c2nccc(Oc3ccc(-n4ncn(Cc5c(F)cccc5F)c4=O)cc3F)c2Cl)C1] 2-(4-((2-(3-amino-3-methylazetidin-1-yl)-3-chloropyridin- 4-yl)oxy)-3-fluorophenyl)-4-(2,6-difluorobenzyl)-2,4- dihydro-3H-1,2,4-triazol-3-one | white solid, yield: 28.7% | ¹H NMR (400 MHz, CDCl₃) δ 7.99 (m, 1H), 7.88 (d, J = 9.0 Hz, 1H), 7.71 (d, J = 4.9 Hz, 1H), 7.40-7.35 (m, 1H), 7.21 (d, J = 8.7 Hz, 1H), 7.01-6.97 (m, 2H), 6.18 (d, J = 6.6 Hz, 1H), 4.98 (s, 2H), 4.50 (d, J = 9.3 Hz, 2H), 3.56 (m, 2H), 1.78 (s, 3H). | 517.1 |
| Example 421 Method 65 | ++++ | 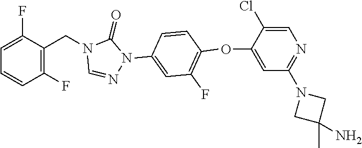 [SMILES: CC1(N)CN(c2cc(Oc3ccc(-n4ncn(Cc5c(F)cccc5F)c4=O)cc3F)c(Cl)cn2)C1] 2-(4-((2-(3-amino-3-methylazetidin-1-yl)-5-chloropyridin- 4-yl)oxy)-3-fluorophenyl)-4-(2,6-difluorobenzyl)-2,4- dihydro-3H-1,2,4-triazol-3-one | white solid, yield: 13.3% | ¹H NMR (400 MHz, CDCl₃) δ 8.05 (s, 1H), 7.82 (m, 2H), 7.64 (s, 1H), 7.39 (s, 1H), 7.23 (d, J = 8.6 Hz, 1H), 6.99 (m, 2H), 5.44 (s, 1H), 4.97 (s, 2H), 3.85 (d, J = 39.5 Hz, 4H), 1.57 (s, 3H). | 517.2 |
| Example 422 Method 65 | ++++ | 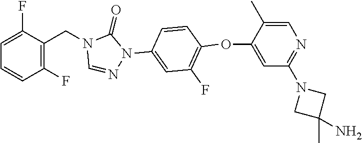 [SMILES: Cc1cnc(N2CC(C)(N)C2)cc1Oc1ccc(-n2ncn(Cc3c(F)cccc3F)c2=O)cc1F] 2-(4-((2-(3-amino-3-methylazetidin-1-yl)-5-methylpyridin- 4-yl)oxy)-3-fluorophenyl)-4-(2,6-difluorobenzyl)-2,4- dihydro-3H-1,2,4-triazol-3-one | white solid, yield: 12.3% | ¹H NMR (400 MHz, CDCl₃) δ 7.99 (d, J = 11.7 Hz, 1H), 7.89 (d, J = 8.8 Hz, 1H), 7.62 (d, J = 7.0 Hz, 2H), 7.39-7.33 (m, 1H), 7.22 (s, 1H), 6.98 (m, 2H), 5.46 (s, 1H), 4.97 (s, 2H), 4.46 (s, 2H), 4.02 (s, 2H), 2.24 (s, 3H), 1.66 (s, 3H). | 497.2 |
| Example 423 Method 65 | ++++ | 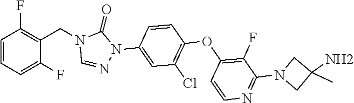 [SMILES: CC1(N)CN(c2nccc(Oc3ccc(-n4ncn(Cc5c(F)cccc5F)c4=O)cc3Cl)c2F)C1] 2-(4-((2-(3-amino-3-methylazetidin-1-yl)-3-fluoropyridin-4- yl)oxy)-3-chlorophenyl)-4-(2,6-difluorobenzyl)-2,4-dihydro- 3H-1,2,4-triazol-3-one | white solid, yield: 18.5% | ¹H NMR (400 MHz, CDCl₃) δ 8.21 (d, J = 2.4 Hz, 1H), 8.00 (m, 1H), 7.63 (s, 1H), 7.59 (s, 1H), 7.37 (m, 1H), 7.23 (d, J = 9.0 Hz, 1H), 6.99 (m, 2H), 6.15 (m, 1H), 4.98 (s, 2H), 4.93 (s, 2H), 4.41 (d, J = 9.3 Hz, 2H), 1.81 (s, 3H). | 517.2 |
| Example 424 Method 65 | +++ | 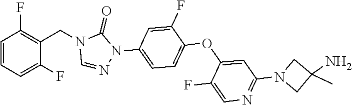 [SMILES: CC1(N)CN(c2cc(Oc3ccc(-n4ncn(Cc5c(F)cccc5F)c4=O)cc3F)c(F)cn2)C1] 2-(4-((2-(3-amino-3-methylazetidin-1-yl)-5-fluoropyridin- 4-yl)oxy)-3-fluorophenyl)-4-(2,6-difluorobenzyl)-2,4-dihydro- 3H-1,2,4-triazol-3-one | white solid, yield: | ¹H NMR (400 MHz, CD₃OD) δ 8.04 (s, 1H), 7.98 (d, J = 3.0 Hz, 1H), 7.93-7.90 (m, 1H), 7.81 (d, J = 7.8 Hz, 1H), 7.52- 7.39 (m, 1H), 7.35-7.32 (m, 1H), 7.06 (t, J = 8.0 Hz, 2H), 5.75 (d, J = 5.5 Hz, 1H), 5.04 (s, 2H), 3.91 (d, J = 8.2 Hz, 2H), 3.85 (d, J = 8.7 Hz, 2H), 1.57 (s, 3H). | 501.1 |

### Example 425: Method 66

### 2-(4-((2-((2S,3S)-3-amino-2-methylazetidin-1-yl)-5-chloropyridin-4-yl)oxy)-3-fluorophenyl)-4-(2,6-difluorobenzyl)-2,4-dihydro-3H-1,2,4-triazol-3-one

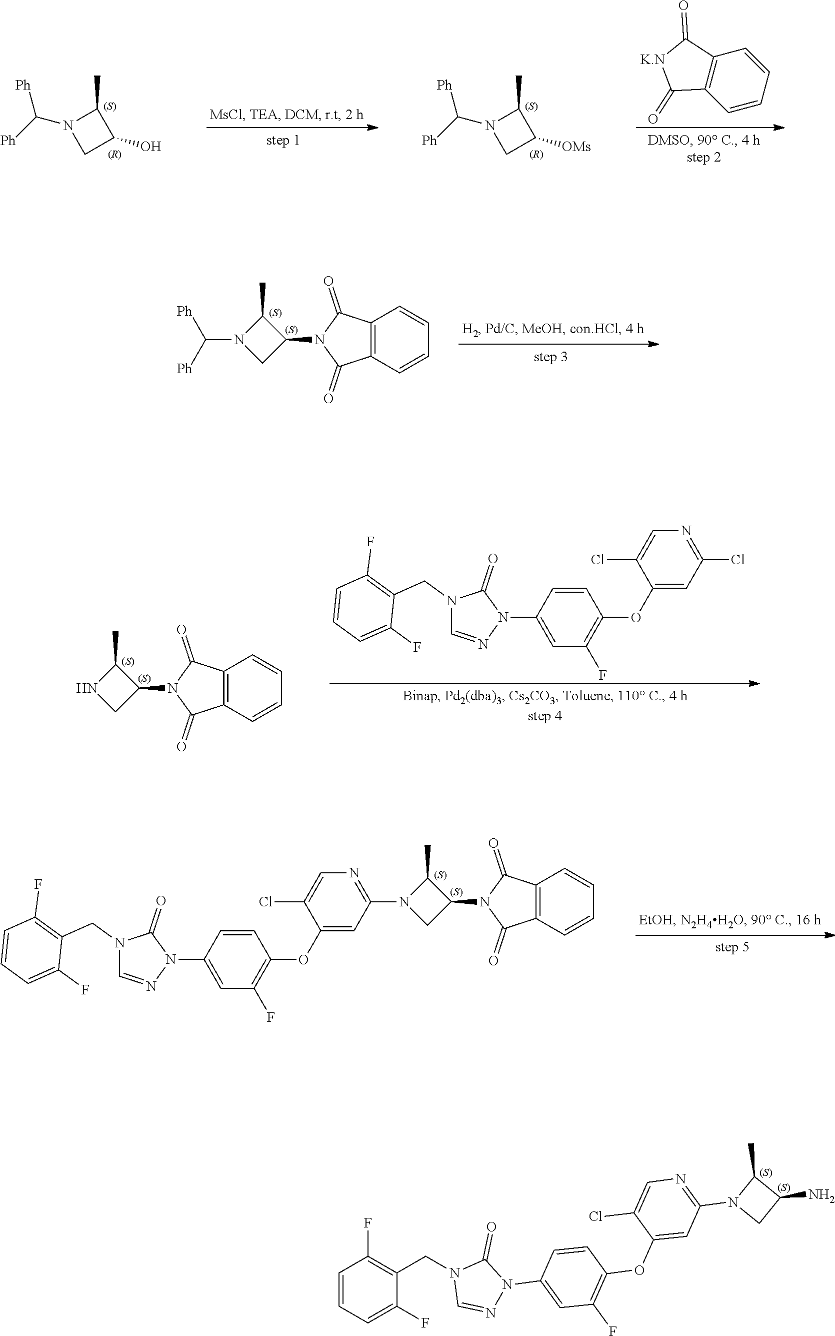

### Step 1: (2S,3R)-1-benzhydryl-2-methylazetidin-3-yl methanesulfonate

To a stirred solution of (2S,3R)-1-(diphenylmethyl)-2-methylazetidin-3-ol (1.8 g, 7.1 mmol) and TEA (3.9 g, 38.6 mol) in DCM (20 mL) was added MsCl (4.63 g, 14.2 mmol). The reaction was stirred at r.t. for 2h, and concentrated to obtain a crude product (2S,3R)-1-benzhydryl-2-methylazetidin-3-yl methanesulfonate (715 mg, 46.4% yield) as a white oil. MS (m/z): 332.1 [M+H]⁺.

### Step 2: 2-((2S,3S)-1-benzhydryl-2-methylazetidin-3-yl)isoindoline-1,3-dione

To a stirred solution of (2S,3R)-1-(diphenylmethyl)-2-methylazetidin-3-yl methanesulfonate (690 mg, 2.0 mmol) in DMSO (15 mL) was added potassium phthalimide (578.4 mg, 3.1 mmol). The reaction was stirred at 90° C. for 4 h under N₂, The mixture was allowed to cool down to rt. The resulted mixture was diluted with water (20 mL). The resulted mixture was extracted with EtOAc (3×10 mL). The combined organic layers were washed with brine (10 mL), dried over anhydrous Na₂SO₄, and after filtration, the filtrate was concentrated under reduced pressure. The residue was purified by silica gel column chromatography, eluted with (EA/PE=1-15%) to afford the titled product (398 mg, 50% yield) as a yellow solid. MS (m/z): 383.0 [M+H]⁺.

### Step 3: 2-((2S,3S)-2-methylazetidin-3-yl)isoindoline-1,3-dione

To a stirred solution of 2-((2S,3S)-1-benzhydryl-2-methylazetidin-3-yl)isoindoline-1,3-dione (398 mg, 1.0 mmol) in MeOH (15 mL) were added Pd/C (1 g, 50% wet) and con. HCl (3d). The reaction mixture was stirred at rt for 4 h under H₂. The reaction mixture was concentrated to afford the crude product 2-((2S,3S)-2-methylazetidin-3-yl)isoindoline-1,3-dione (100 mg, 44% yield) as a yellow oil. MS (m/z): 217.2 [M+H]⁺.

### Step 4: 2-((2S,3S)-1-(5-chloro-4-(4-(4-(2,6-difluorobenzyl)-5-oxo-4,5-dihydro-1H-1,2,4-triazol-1-yl)-2-fluorophenoxy)pyridin-2-yl)-2-methylazetidin-3-yl)isoindoline-1,3-dione

To a stirred solution of 2-{4-[(2,5-dichloropyridin-4-yl)oxy]-3-fluorophenyl}-4-[(2,6-difluorophenyl)methyl]-1,2,4-triazol-3-one (80 mg, 0.17 mmol) and 2-[(2S,3S)-2-methylazetidin-3-yl]isoindole-1,3-dione (74.0 mg, 0.34 mmol) in toluene (20 mL) were added BINAP (12.1 mg, 0.08 mmol), tris(dibenzylideneacetone)dipalladium (16.3 mg, 0.08 mmol) and Cs₂CO₃ (94.6 mg, 0.68 mmol). The reaction was stirred at 110° C. for 4 h under N₂. The mixture was allowed to cool down to rt. The resulted mixture was diluted with water (20 mL). The resulted mixture was extracted with EtOAc (3×10 mL). The combined organic layers were washed with brine (10 mL), dried over anhydrous Na₂SO₄, and after filtration, the filtrate was concentrated under reduced pressure. The residue was purified by silica gel column chromatography, eluted with (MeOH/DCM=1-10%) to afford the titled product 2-((2S,3S)-1-(5-chloro-4-(4-(4-(2,6-difluorobenzyl)-5-oxo-4,5-dihydro-1H-1,2,4-triazol-1-yl)-2-fluorophenoxy)pyridin-2-yl)-2-methylazetidin-3-yl)isoindoline-1,3-dione (50 mg, 16.7% yield) as a white solid. MS (m/z): 647.0 [M+H]⁺.

### Step 5: 2-(4-((2-((2S,3S)-3-amino-2-methylazetidin-1-yl)-5-chloropyridin-4-yl)oxy)-3-fluorophenyl)-4-(2,6-difluorobenzyl)-2,4-dihydro-3H-1,2,4-triazol-3-one

To a stirred solution of 2-[(2S,3S)-1-[5-chloro-4-(4-{4-[(2,6-difluorophenyl)methyl]-5-oxo-1,2,4-triazol-1-yl}-2-fluorophenoxy)pyridin-2-yl]-2-methylazetidin-3-yl]isoindole-1,3-dione (50 mg, 0.07 mmol) in EtOH (20 mL) was added N₂H₄·H₂O (147.2 mg, 0.72 mmol) in a sealed tube. The reaction was stirred at 90° C. for 16 h. The resulted mixture was diluted with water (20 mL). The resulted mixture was extracted with EtOAc (3×10 mL). The combined organic layers were washed with brine (10 mL), dried over anhydrous Na₂SO₄, and after filtration, the filtrate was concentrated under reduced pressure. The residue was purified by silica gel column chromatography, eluted with (EA/PE=1-15%) to afford the titled product 2-(4-((2-((2S,3S)-3-amino-2-methylazetidin-1-yl)-5-chloropyridin-4-yl)oxy)-3-fluorophenyl)-4-(2,6-difluorobenzyl)-2,4-dihydro-3H-1,2,4-triazol-3-one (9 mg, 19.2% yield) as a white solid. MS (m/z): 517.0 [M+H]⁺. ¹H NMR (400 MHz, CDCl₃) δ 8.06 (s, 1H), 7.88 (d, J=11.7 Hz, 1H), 7.80 (d, J=8.9 Hz, 1H), 7.64 (s, 1H), 7.41-7.34 (m, 1H), 7.22 (t, J=8.8 Hz, 1H), 7.00 (t, J=7.9 Hz, 2H), 5.49 (s, 1H), 4.96 (s, 2H), 4.11 (dd, J=20.6, 13.6 Hz, 1H), 3.60-3.48 (m, 3H), 1.41 (d, J=6.2 Hz, 3H).

The synthesis procedure of Example (Compound) 426 was similar to the procedure of Example 425.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 425 Method 66 | ++++ | 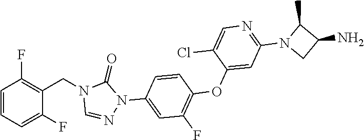 [SMILES: C[C@H]1[C@@H](N)CN1c1cc(Oc2ccc(-n3ncn(Cc4c(F)cccc4F)c3=O)cc2F)c(Cl)cn1] 2-(4-((2-((2S,3S)-3-amino-2-methylazetidin-1-yl)-5- chloropyridin-4-yl)oxy)-3-fluorophenyl)-4-(2,6- difluorobenzyl)-2,4-dihydro-3H-1,2,4-triazol-3-one | white solid, yield: 19.2% | ¹H NMR (400 MHz, CDCl₃) δ 8.06 (s, 1H), 7.88 (d, J = 11.7 Hz, 1H), 7.80 (d, J = 8.9 Hz, 1H), 7.64 (s, 1H), 7.41- 7.34 (m, 1H), 7.22 (t, J = 8.8 Hz, 1H), 7.00 (t, J = 7.9 Hz, 2H), 5.49 (s, 1H), 4.96 (s, 2H), 4.11 (dd, J = 20.6, 13.6 Hz, 1H), 3.60-3.48 (m, 3H), 1.41 (d, J = 6.2 Hz, 3H). | 517.0 |
| Example 426 Method 66 | ++++ | 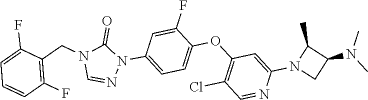 [SMILES: C[C@H]1[C@@H](N(C)C)CN1c1cc(Oc2ccc(-n3ncn(Cc4c(F)cccc4F)c3=O)cc2F)c(Cl)cn1] 2-(4-((5-chloro-2-((2S,3S)-3-(dimethylamino)-2-methylazetidin- 1-yl)pyridin-4-yl)oxy)-3-fluorophenyl)-4-(2,6-difluorobenzyl)- 2,4-dihydro-3H-1,2,4-triazol-3-one | yellow solid, yield: 21% | ¹H NMR (400 MHz, CDCl₃) δ 8.09 (s, 1H), 7.99 (dd, 1H), 7.86 (dt, J = 9.0 Hz, 1H), 7.62 (s, 1H), 7.38 (m, 1H), 7.22 (t, 1H), 7.04-6.95 (t, 2H), 5.52 (s, 1H), 4.99 (s, 2H), 3.99 (s, 1H), 2.41 (s, 6H), 1.59 (m, 3H), 1.43 (s, 3H). | 545.2 |

### Example 427: Method 67

### 4-benzyl-5-chloro-2-(4-(pyridin-3-yloxy)phenyl)-2,4-dihydro-3H-1,2,4-triazol-3-one

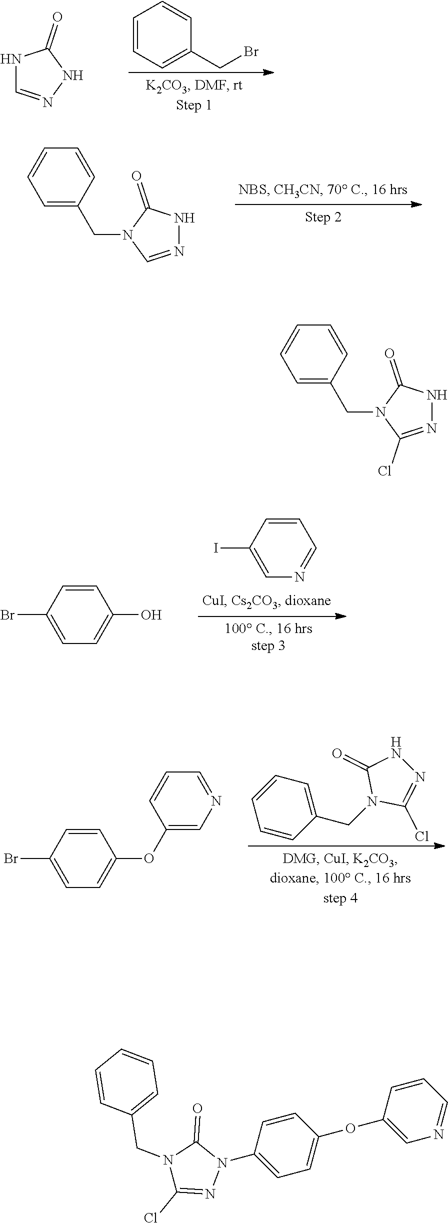

### Step 1: 4-benzyl-2,4-dihydro-3H-1,2,4-triazol-3-one

At 0° C., to the solution of 2,4-dihydro-3H-1,2,4-triazol-3-one (50 g, 0.59 mol) in DMF(1000 mL) was slowly added dropwise Benzyl bromide (102.6 g, 0.6 mol). After that, the mixture was stirred at r.t. for 16 hours. The solvent was removed in vacuo. To the residue was added tert-Butyl methyl ether (200 mL) and water (200 mL), and the resulted mixture was stirred for min. The solid was collected by filtration to give 4-benzyl-2,4-dihydro-3H-1,2,4-triazol-3-one (60 g, yield: 58%) as white solid. LC-MS (m/z): 176.1[M+H]⁺.

### Step 2: 4-benzyl-5-chloro-2,4-dihydro-3H-1,2,4-triazol-3-one

A mixture of 4-benzyl-2,4-dihydro-3H-1,2,4-triazol-3-one (20 g, 114.3 mmol) and NBS (40 g, 228.6 mmol) in Acetonitrile (350 mL) was stirred at 70° C. for 12 hours under nitrogen atmosphere. After cooling down, the mixture was removed in vacuo. The residue was purified by column chromatography on silica gel (eluting Petroleum ether: Ethyl acetate=10:1 to 2:1) to give 4-benzyl-5-chloro-2,4-dihydro-3H-1,2,4-triazol-3-one (15 g, yield: 62%) as a white solid. LC-MS (m/z): 210.1[M+H]⁺. ¹H NMR (400 MHz, CDCl₃) δ ppm 10.46 (s, 1H), 7.35 (m, 5H), 4.87 (s, 2H).

### Step 3: 3-(4-bromophenoxy)pyridine

A solution of 4-bromophenol (500 mg, 2.9 mmol), 3-iodopyridine (592 mg, 2.9 mmol), 1-(pyridin-2-yl)propan-2-one (39 mg, 0.3 mmol), and Cs₂CO₃ (1.88 g, 5.8 mmol) in dioxane (15 mL) was stirred at 100° C. for 16 h. The resulted mixture was diluted with water (20 mL). The resulted mixture was extracted with EtOAc (3×10 mL). The combined organic layers were washed with brine (20 mL), dried over anhydrous Na₂SO₄, and after filtration, the filtrate was concentrated under reduced pressure. The residue was purified by Combi-Flash (EtOAc: PE=0:1 to 1:3) to afford 3-(4-bromophenoxy)pyridine (400 mg, 49.8% yield) as a colorless oil. LC-MS (m/z): 252.0 [M+H]⁺.

### Step 4: 4-benzyl-5-chloro-2-(4-(pyridin-3-yloxy)phenyl)-2,4-dihydro-3H-1,2,4-triazol-3-one

A solution of 4-benzyl-5-chloro-2H-1,2,4-triazol-3-one (100 mg, 0.5 mmol), 3-(4-bromophenoxy)pyridine (143 mg, 0.6 mmol), DMG, and CuI (9 mg, 0.05 mmol), K₂CO₃(132 mg, 1 mmol) in dioxane (5 mL) was stirred at 110° C. for 16 h. The resulted mixture was diluted with water (20 mL). It was extracted with EtOAc (3×10 mL). The combined organic layers were washed with brine (20 mL), dried over anhydrous Na₂SO₄, and after filtration, the filtrate was concentrated under reduced pressure. The residue was purified by prep-HPLC to afford the titled product (17.1 mg, 9.1% yield) as a white solid. LC-MS (m/z): 379.1 [M+H]⁺. ¹H NMR (400 MHz, DMSO-d6) δ 8.40 (d, J 7.5 Hz, 2H), 7.87 (d, J 9.1 Hz, 2H), 7.50-7.31 (m, 7H), 7.20 (d, J=9.1 Hz, 2H), 4.95 (s, 2H).

The synthesis procedure of Examples (Compounds) 428-430 was similar to the procedure of Example 427.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 427 Method 67 | ++++ | 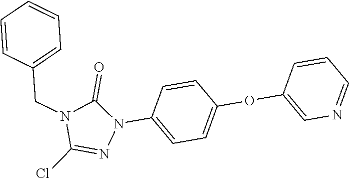 [SMILES: O=c1n(-c2ccc(Oc3cccnc3)cc2)nc(Cl)n1Cc1ccccc1] 4-benzyl-5-chloro-2-(4-(pyridin-3-yloxy)phenyl)-2,4- dihydro-3H-1,2,4-triazol-3-one | off-white solid, yield: 9.1% | ¹H NMR (400 MHz, DMSO-d₆) δ 8.40 (d, J = 7.5 Hz, 2H), 7.87 (d, J = 9.1 Hz, 2H), 7.50-7.31 (m, 7H), 7.20 (d, J = 9.1 Hz, 2H), 4.95 (s, 2H). | 379.1 |
| Example 428 Method 67 | ++++ | 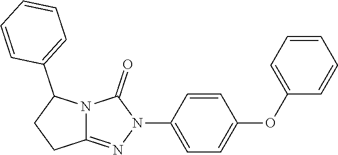 [SMILES: O=c1n(-c2ccc(Oc3ccccc3)cc2)nc2n1C(c1ccccc1)CC2] 2-(4-phenoxyphenyl)-5-phenyl-2,5,6,7-tetrahydro-3H- pyrrolo[2,1-c][1,2,4]triazol-3-one | white solid, yield: 1.9% | ¹H NMR (400 MHz, CDCl₃) δ 7.91-7.85 (m, 2H), 7.41-7.29 (m, 5H), 7.27-7.26 (m, 1H), 7.24 (ddd, J = 2.4, 1.2, 0.7 Hz, 1H), 7.10-6.97 (m, 5H), 5.28 (dt, J = 8.0, 3.8 Hz, 1H), 3.11- 2.86 (m, 3H), 2.51 (s, 1H). | 370.2 |
| Example 429 Method 67 | +++ | 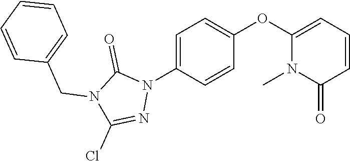 [SMILES: Cn1c(Oc2ccc(-n3nc(Cl)n(Cc4ccccc4)c3=O)cc2)cccc1=O] 6-(4-(4-benzyl-3-chloro-5-oxo-4,5-dihydro-1H-1,2,4-triazol- 1-yl)phenoxy)-1-methylpyridin-2(1H)-one | white solid, yield: 20% | ¹H NMR (400 MHz, DMSO-d₆) δ 7.95 (m, 2H), 7.38 (m, 7H), 7.32 (m, 1H), 6.11 (dd, J = 9.1, 0.9 Hz, 1H), 5.29 (dd, J = 7.7, 1.0 Hz, 1H), 4.96 (s, 2H), 3.49 (s, 3H). | 408.9 |
| Example 430 Method 67 | +++ | 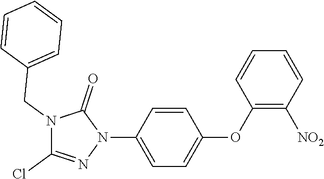 [SMILES: O=c1n(-c2ccc(Oc3ccccc3[N+](=O)[O-])cc2)nc(Cl)n1Cc1ccccc1] 4-benzyl-5-chloro-2-(4-(2-nitrophenoxy)phenyl)-2,4- dihydro-3H-1,2,4-triazol-3-one | yellow solid, yield: 22% | ¹H NMR (400 MHz, DMSO-d₆) δ ppm 8.09 (dd, J = 8.2, 1.6 Hz, 1H), 7.87 (m, 2H), 7.72 (ddd, J = 8.4, 7.5, 1.7 Hz, 1H), 7.39 (m, 6H), 7.21 (m, 3H), 4.95 (s, 2H). | 445.1 |

### Example 431: Method 68

### 4-(2,6-difluorobenzyl)-2-(4-(pyridazin-4-yloxy)phenyl)-2,4-dihydro-3H-1,2,4-triazol-3-one

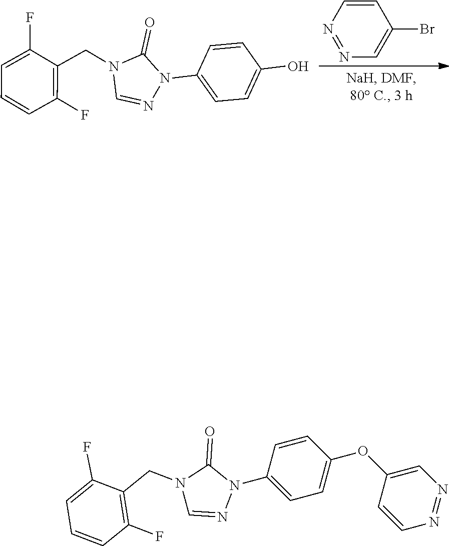 [SMILES: O=c1n(Cc2c(F)cccc2F)cnn1-c1ccc(O)cc1 ; Brc1ccnnc1 ; O=c1n(Cc2c(F)cccc2F)cnn1-c1ccc(Oc2ccnnc2)cc1]

### 4-(2,6-difluorobenzyl)-2-(4-(pyridazin-4-yloxy)phenyl)-2,4-dihydro-3H-1,2,4-triazol-3-one

A solution of 4-[(2,6-difluorophenyl)methyl]-2-(4-hydroxyphenyl)-1,2,4-triazol-3-one (100 mg, 0.32 mmol), 4-bromopyridazine (52 mg, 0.33 mmol), and NaH (26 mg, 0.66 mmol) in DMF (3 mL) was stirred at 80° C. for 3h. It was diluted with water (20 mL) and extracted with EtOAc (3×10 mL). The combined organic layers were washed with brine (20 mL), and dried over anhydrous Na₂SO₄. After filtration, the filtrate was concentrated under reduced pressure. The residue was purified by prep-HPLC to afford the titled product (12.0 mg, 9.2% yield) as a white solid. LC-MS (m/z): 382.0 [M+H]⁺. ¹H NMR (400 MHz, CDCl₃) δ 9.30 (d, J=6.2 Hz, 1H), 9.07 (d, J=3.0 Hz, 1H), 8.16-8.11 (m, 1H), 7.63 (s, 1H), 7.42-7.34 (m, 4H), 7.19-7.13 (m, 1H), 7.04-6.96 (m, 2H), 5.00 (s, 2H).

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 431 Method 68 | ++ | 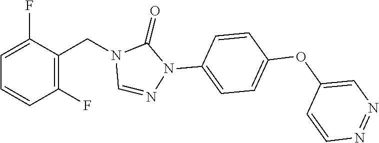 [SMILES: O=c1n(Cc2c(F)cccc2F)cnn1-c1ccc(Oc2ccnnc2)cc1] 4-(2,6-difluorobenzyl)-2-(4-(pyridazin-4-yloxy)phenyl)-2,4- dihydro-3H-1,2,4-triazol-3-one | white solid, yield: 9.2% | ¹H NMR (400 MHz, CDCl₃) δ 9.30 (d, J = 6.2 Hz, 1H), 9.07 (d, J = 3.0 Hz, 1H), 8.16-8.11 (m, 1H), 7.63 (s, 1H), 7.42-7.34 (m, 4H), 7.19-7.13 (m, 1H), 7.04- 6.96 (m, 2H), 5.00 (s, 2H) | 382.0 |

### Example 432: Method 69

### 2-(4-((3-bromopyridin-4-yl)oxy)phenyl)-4-(2,6-difluorobenzyl)-2,4-dihydro-3H-1,2,4-triaz ol-3-one

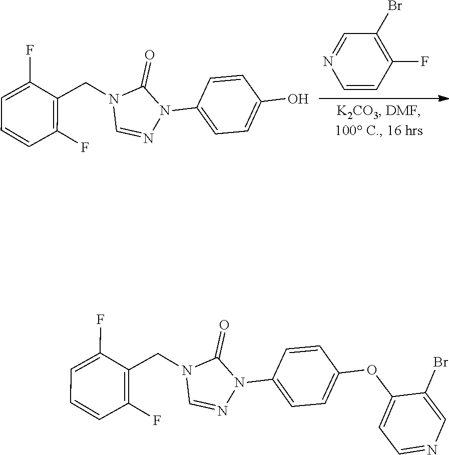 [SMILES: O=c1n(Cc2c(F)cccc2F)cnn1-c1ccc(O)cc1 ; Fc1ccncc1Br ; O=c1n(Cc2c(F)cccc2F)cnn1-c1ccc(Oc2ccncc2Br)cc1]

### 2-(4-((3-bromopyridin-4-yl)oxy)phenyl)-4-(2,6-difluorobenzyl)-2,4-dihydro-3H-1,2,4-triaz ol-3-one

A solution of 4-[(2,6-difluorophenyl)methyl]-2-(4-hydroxyphenyl)-1,2,4-triazol-3-one (100 mg, 0.3 mmol), 2-bromo-4-fluoropyridine (87 mg, 0.5 mmol), K₂CO₃ (91 mg, 0.65 mmol) in DMF (5 mL) was stirred at 100° C. for 16 h. The resulted mixture was diluted with water (20 mL). The resulted mixture was extracted with EtOAc (3×10 mL). The combined organic layers were washed with brine (20 mL), dried over anhydrous Na₂SO₄, and after filtration, the filtrate was concentrated under reduced pressure. The residue was purified by prep-TLC (EtOAc. PE=1: 1) to afford the titled product (15.1 mg, 9.1% yield) as a white solid. [M+H]⁺=461. ¹H NMR (400 MHz, CDCl₃) δ 8.71 (d, J=3.9 Hz, 1H), 8.32 (d, J=4.2 Hz, 1H), 8.07 (dd, J=7.8, 5.4 Hz, 2H), 7.61 (s, 1H), 7.37 (dd, J=14.9, 6.7 Hz, 1H), 7.17 (dd, J=8.9, 2.1 Hz, 2H), 7.00 (dd, J=13.5, 5.7 Hz, 2H), 6.70-6.60 (m, 1H), 5.00 (s, 2H).

The synthesis procedure of Examples (Compounds) 433-438 was similar to the procedure of Example 432.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 432 Method 69 | ++++ | 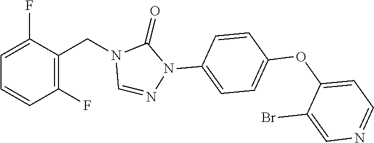 [SMILES: O=c1n(Cc2c(F)cccc2F)cnn1-c1ccc(Oc2ccncc2Br)cc1] 2-(4-((3-bromopyridin-4-yl)oxy)phenyl)-4-(2,6-difluorobenzyl)- 2,4-dihydro-3H-1,2,4-triazol-3-one | white solid, yield: 9.1% | ¹H NMR (400 MHz, CDCl₃) δ 8.71 (d, J = 3.9 Hz, 1H), 8.32 (d, J = 4.2 Hz, 1H), 8.07 (dd, J = 7.8, 5.4 Hz, 2H), 7.61 (s, 1H), 7.37 (dd, J = 14.9, 6.7 Hz, 1H), 7.17 (dd, J = 8.9, 2.1 Hz, 2H), 7.00 (dd, J = 13.5, 5.7 Hz, 2H), 6.70- 6.60 (m, 1H), 5.00 (s, 2H). | 459.0, 461.0 |
| Example 433 Method 69 | +++ | 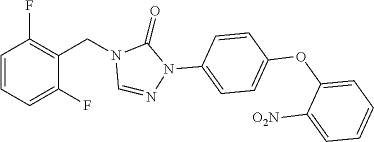 [SMILES: O=c1n(Cc2c(F)cccc2F)cnn1-c1ccc(Oc2ccccc2[N+](=O)[O-])cc1] 4-(2,6-difluorobenzyl)-2-(4-(2-nitrophenoxy)phenyl)-2,4- dihydro-3H-1,2,4-triazol-3-one | white solid | NA | 425.1 |
| Example 434 Method 69 | ++++ | 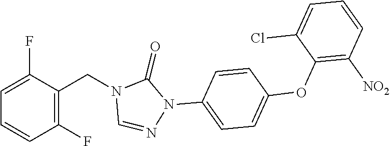 [SMILES: O=c1n(Cc2c(F)cccc2F)cnn1-c1ccc(Oc2c(Cl)cccc2[N+](=O)[O-])cc1] 2-(4-(2-chloro-6-nitrophenoxy)phenyl)-4-(2,6-difluorobenzyl)- 2,4-dihydro-3H-1,2,4-triazol-3-one | white solid, | ¹H NMR (400 MHz, CDCl₃) δ 7.93-7.86 (m, 3H), 7.73 (dd, J = 8.2, 1.6 Hz, 1H), 7.56 (s, 1H), 7.42-7.31 (m, 2H), 6.98 (p, J = 3.6 Hz, 2H), 6.91-6.82 (m, 2H), 4.97 (s, 2H) | 459.1 |
| Example 435 Method 69 | +++ | 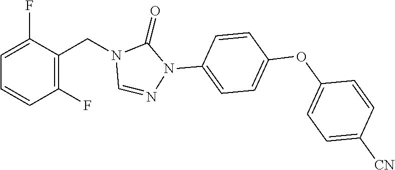 [SMILES: N#Cc1ccc(Oc2ccc(-n3ncn(Cc4c(F)cccc4F)c3=O)cc2)cc1] 4-(4-(4-(2,6-difluorobenzyl)-5-oxo-4,5-dihydro-1H-1,2,4-triazol- 1-yl)phenoxy)benzonitrile | white solid, yield: 20.1% | ¹H NMR (400 MHz, CDCl₃) δ 8.04-7.97 (m, 1H), 7.64- 7.57 (m, 2H), 7.37 (ddd, J = 13.1, 7.5, 4.2 Hz, 1H), 7.14- 7.08 (m, 1H), 7.04-6.95 (m, 2H), 4.99 (s, 2H). | 405.1 |
| Example 436 Method 69 | +++ | 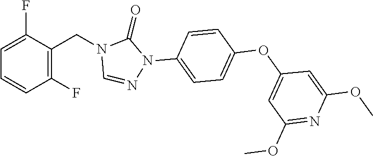 [SMILES: COc1cc(Oc2ccc(-n3ncn(Cc4c(F)cccc4F)c3=O)cc2)cc(OC)n1] 4-(2,6-difluorobenzyl)-2-(4-((2,6-dimethoxypyridin-4-yl)oxy) phenyl)-2,4-dihydro-3H-1,2,4-triazol-3-one | white solid, yield: 20.1% | ¹H NMR (400 MHz, CDCl₃) δ 7.96 (m, 2H), 7.59 (s, 1H), 7.37 (tt, J = 8.4, 6.5 Hz, 1H), 7.19 (m, 2H), 6.99 (m, 2H), 5.96 (d, J = 1.8 Hz, 1H), 5.88 (d, J = 1.8 Hz, 1H), 4.99 (s, 2H), 3.78 (d, J = 4.1 Hz, 6H). | 441.1 |
| Example 437 Method 69 | ++++ | 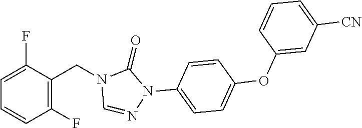 [SMILES: N#Cc1cccc(Oc2ccc(-n3ncn(Cc4c(F)cccc4F)c3=O)cc2)c1] 3-(4-(4-(2,6-difluorobenzyl)-5-oxo-4,5-dihydro-1H-1,2,4- triazol-1-yl)phenoxy)benzonitrile | | ¹H NMR (400 MHz, CDCl₃) δ 8.01-7.96 (m, 2H), 7.58 (s, 1H), 7.43-7.33 (m, 4H), 7.22- 7.17 (m, 2H), 7.09-7.04 (m, 1H), 7.01-6.95 (m, 2H), 4.98 (s, 2H). | 405.1 |
| Example 438 Method 69 | +++ | 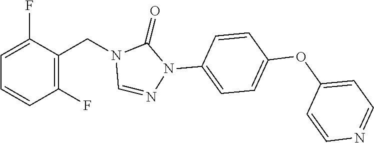 [SMILES: O=c1n(Cc2c(F)cccc2F)cnn1-c1ccc(Oc2ccncc2)cc1] 4-(2,6-difluorobenzyl)-2-(4-(pyridin-4-yloxy)phenyl)-2,4- dihydro-3H-1,2,4-triazol-3-one | white solid, yield: 19.1% | ¹H NMR (400 MHz, DMSO- d₆) δ 8.46 (dd, J = 4.7, 1.6 Hz, 2H), 8.33 (s, 1H), 7.99- 7.92 (m, 2H), 7.53-7.43 (m, 1H), 7.31-7.24 (m, 2H), 7.21-7.12 (m, 2H), 6.93 (dd, J = 4.7, 1.6 Hz, 2H), 4.99 (s, 2H). | 381.1 |

### Example 439: Method 70

### 2-(4-(2-aminophenoxy)phenyl)-4-(2,6-difluorobenzyl)-2,4-dihydro-3H-1,2,4-triazol-3-one

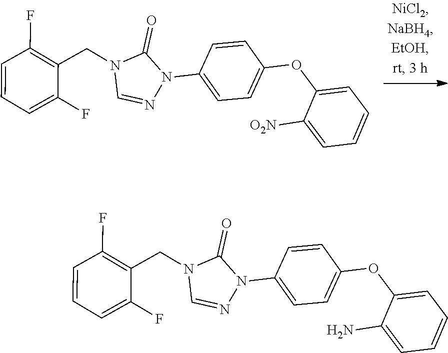 [SMILES: O=c1n(Cc2c(F)cccc2F)cnn1-c1ccc(Oc2ccccc2[N+](=O)[O-])cc1 ; Nc1ccccc1Oc1ccc(-n2ncn(Cc3c(F)cccc3F)c2=O)cc1]

To a solution of 4-(2,6-difluorobenzyl)-2-(4-(2-nitrophenoxy)phenyl)-2,4-dihydro-3H-1,2,4-triazol-3-one (90 mg, 0.21 mmol) in EtOH (10 mL) was added NiCl₂ (82 mg 0.64 mmol). The mixture was stirred at RT for 10 min and NaBH₄ (24 mg 0.64 mmol) was added. It was then stirred at 25° C. for 3 h. The resulted mixture was diluted with water (40 mL). The resulted mixture was extracted with EtOAc (3×20 mL). The combined organic layers were washed with brine (40 mL), dried over anhydrous Na₂SO₄, and after filtration, the filtrate was concentrated under reduced pressure. The residue was purified by reverse phase flash chromatography with the following conditions: column: Spherical C18 40-60 um, 40 g; Mobile phase B: ACN; Flow rate: 40 mL/min; Gradient: 35% B-60% B in 20 min; Detector: 254 nm. The fractions containing the desired product were collected at 51% B and concentrated under reduced pressure to afford the titled compound (20 mg, 23.9%) as an off-white solid. LC-MS (m/z): 395.1 [M+H]⁺. ¹H NMR (400 MHz, DMSO) δ 8.26 (s, 1H), 7.76 (d, J=9.1 Hz, 2H), 7.53-7.42 (m, 1H), 7.16 (t, J=8.1 Hz, 2H), 7.01-6.88 (m, 3H), 6.85-6.75 (m, 2H), 6.60-6.50 (m, 1H), 4.96 (s, 2H).

The synthesis procedure of Examples (Compounds) 440-443 was similar to the procedure of Example 439.

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 439 Method 70 | ++++ | 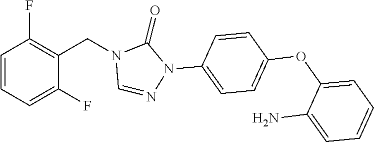 [SMILES: Nc1ccccc1Oc1ccc(-n2ncn(Cc3c(F)cccc3F)c2=O)cc1] 2-(4-(2-aminophenoxy)phenyl)-4-(2,6-difluorobenzyl)-2,4- dihydro-3H-1,2,4-triazol-3-one | white solid, yield: 9.1% | ¹H NMR (400 MHz, DMSO- d₆) δ 8.26 (s, 1H), 7.76 (d, J = 9.1 Hz, 2H), 7.53-7.42 (m, 1H), 7.16 (t, J = 8.1 Hz, 2H), 7.01-6.88 (m, 3H), 6.85-6.75 (m, 2H), 6.60-6.50 (m, 1H), 4.96 (s, 2H). | 395.1 |
| Example 440 Method 70 | ++++ | 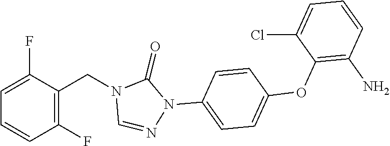 [SMILES: Nc1cccc(Cl)c1Oc1ccc(-n2ncn(Cc3c(F)cccc3F)c2=O)cc1] 2-(4-(2-amino-6-chlorophenoxy)phenyl)-4-(2,6-difluorobenzyl)- 2,4-dihydro-3H-1,2,4-triazol-3-one | white solid, | ¹H NMR (400 MHz, CDCl₃) δ 7.87-7.79 (m, 2H), 7.54 (s, 1H), 7.42-7.30 (m, 1H), 6.99- 6.93 (m, 4H), 6.85 (dd, J = 8.0, 1.4 Hz, 1H), 6.76 (dd, J = 8.0, 1.2 Hz, 1H), 4.96 (s, 2H). | 429.1 |
| Example 441 Method 70 | ++++ | 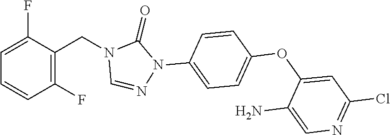 [SMILES: Nc1cnc(Cl)cc1Oc1ccc(-n2ncn(Cc3c(F)cccc3F)c2=O)cc1] 2-(4-((5-amino-2-chloropyridin-4-yl)oxy)phenyl)-4-(2,6- difluorobenzyl)-2,4-dihydro-3H-1,2,4-triazol-3-one | | ¹H NMR (400 MHz, CDCl₃) δ 8.09-8.00 (m, 2H), 7.87 (s, 1H), 7.60 (s, 1H), 7.36 (tt, J = 8.4, 6.5 Hz, 1H), 7.17- 7.10 (m, 2H), 7.02-6.94 (m, 2H), 6.49 (s, 1H), 4.98 (s, 2H). | 429.6 |
| Example 442 Method 70 | ++++ | 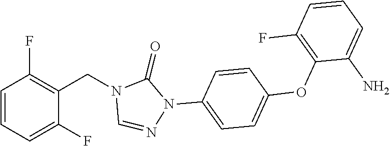 [SMILES: Nc1cccc(F)c1Oc1ccc(-n2ncn(Cc3c(F)cccc3F)c2=O)cc1] 2-(4-(2-amino-6-fluorophenoxy)phenyl)-4-(2,6-difluorobenzyl)- 2,4-dihydro-3H-1,2,4-triazol-3-one | white solid, yield: 26.9% | ¹H NMR (400 MHz, DMSO- d₆) δ 7.86-7.80 (m, 2H), 7.52 (d, J = 8.6 Hz, 1H), 7.40-7.30 (m, 1H), 7.00-6.91 (m, 5H), 6.61-6.50 (m, 2H), 4.97 (d, J = 9.6 Hz, 2H). | 413.4 |
| Example 443 Method 70 | +++ | 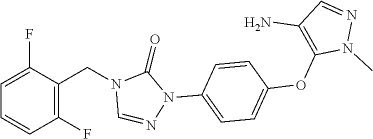 [SMILES: Cn1ncc(N)c1Oc1ccc(-n2ncn(Cc3c(F)cccc3F)c2=O)cc1] 2-(4-((4-amino-1-methyl-1H-pyrazol-5-yl)oxy)phenyl)-4-(2,6- difluorobenzyl)-2,4-dihydro-3H-1,2,4-triazol-3-one | white solid, yield: | ¹H NMR (400 MHz, CDCl₃) δ 7.92-7.87 (m, 2H), 7.57 (s, 1H), 7.42-7.32 (m, 1H), 7.22 (s, 1H), 7.03-6.94 (m, 4H), 4.97 (s, 2H), 3.60 (s, 3H). | 399.1 |

### Example 444: Method 71

### 4-benzyl-5-chloro-2-[4-(pyridin-4-yloxy)phenyl]-1,2,4-triazol-3-one

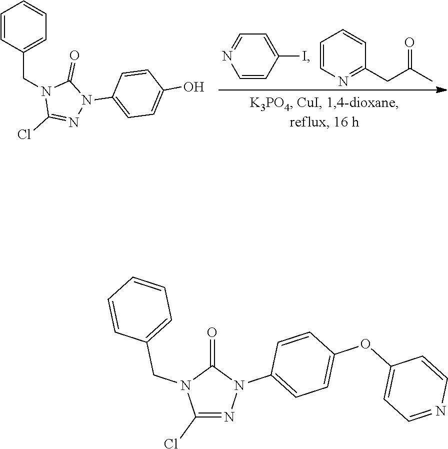

To a stirred 4-benzyl-5-chloro-2-(4-hydroxyphenyl)-1,2,4-triazol-3-one (20 mg, 0.0663 mmol), 4-iodopyridine (31.95 mg, 0.0663 mmol), CuI (2.52 mg, 0.0132 mmol), 1-(pyridin-2-yl)propan-2-one (1.80 mg, 0.0132 mmol) in 1,4-dioxane was added K₃PO₄(35.18 mg, 0.1657 mmol). The resulted mixture was stirred for additional 16 hrs at 120° C. After filtration, the filtrate was concentrated under reduced pressure. The resulted mixture was diluted with water (100 mL). The resulted mixture was extracted with EtOAc (3×50 mL). The combined organic layers were washed with brine (100 mL), dried over anhydrous Na₂SO₄. After filtration, the filtrate was concentrated under reduced pressure. The residue was purified by flash (PE:EA=2:1) to obtain the titled compound (4 mg, 15.92% yield) as a white solid. LC-MS (m/z): 379.8 [M+H]⁺. ¹H NMR (400 MHz, MeOD) δ 8.11-8.01 (m, 2H), 7.65-7.53 (m, 7H), 7.44-7.36 (m, 4H), 4.90 (s, 2H).

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 444 Method 71 | +++ | 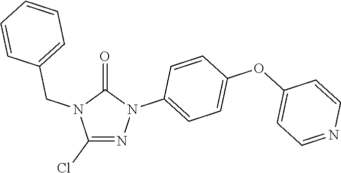 [SMILES: O=c1n(-c2ccc(Oc3ccncc3)cc2)nc(Cl)n1Cc1ccccc1] 4-benzyl-5-chloro-2-(4-(pyridin-4-yloxy)phenyl)-2,4- dihydro-3H-1,2,4-triazol-3-one | white solid, yield: 15.92% | ¹H NMR (400 MHz, MeOD) δ 8.11-8.01 (m, 2H), 7.65- 7.53 (m, 7H), 7.44-7.36 (m, 4H), 4.90 (s, 2H). | 379.8 |

### Example 445: Method 72

### (R)-5-(2-fluoro-4-(3-oxo-5-phenyl-5,6-dihydrooxazolo[2,3-c][1,2,4]triazol-2(3H)-yl)phenoxy)-4-methylthiazole-2-carboxamide

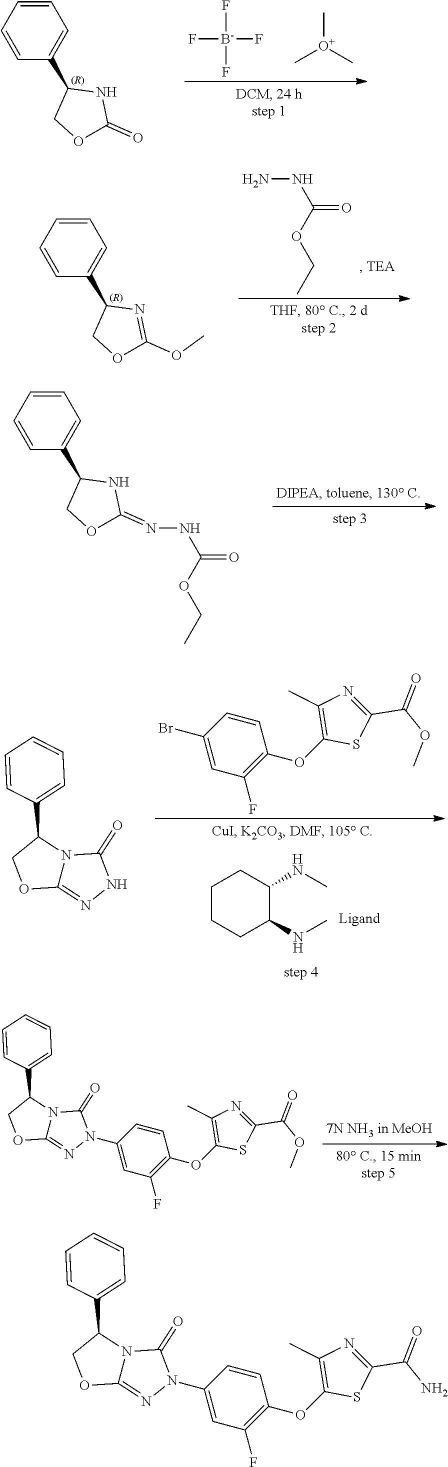

### Step 1: (R)-2-methoxy-4-phenyl-4,5-dihydrooxazole

To a stirred solution of (4R)-4-phenyloxazolidin-2-one (1.00 eq, 5.00 g, 30.6 mmol) in DCM (50 mL) was added trimethyloxonium tetrafluoroborate (1.20 eq, 5439 mg, 36.8 mmol) at room temperature. After addition, the mixture was stirred at 25° C. for 12 h under N₂. LCMS indicated the reaction was completed. Then, to the reaction mixture was added cooled NaHCO₃ aq. (50 mL) and extracted with DCM (30 mL×3). The organics were then combined and dried (over Na₂SO₄) before concentration to dryness. The crude was directly used for next step without purification. MS (m/z): 178.1 [M+H]⁺.

### Step 2: ethyl (R)-2-(4-phenyloxazolidin-2-ylidene)hydrazine-1-carboxylate

To a solution of (R)-2-methoxy-4-phenyl-4,5-dihydrooxazole (1.00 eq, 5.00 g, 28.2 mmol) in THF (50 mL), were added TEA (1.20 eq, 4.7 mL, 33.9 mmol) and ethyl N-aminocarbamate (1.20 eq, 3525 mg, 33.9 mmol) at room temperature. After addition, the mixture was stirred at 80° C. for 2 d. LCMS indicated the reaction was completed. Then the reaction mixture was cooled and concentrated to dryness. The crude was purified with silica gel column (EtOAc/MeOH=10/1) to give the product (3 g) and purified with C18 column (ACN/water=5% to 80%) to give the product (1.30 g, 4.43 mmol, 15.71% yield) as a yellow gum. MS (m/z): 250.1 [M+H]⁺. ¹H NMR (400 MHz, DMSO-d₆) δ 8.95 (s, 1H), 8.58 (s, 1H), 7.46-7.18 (m, 5H), 4.99-4.90 (m, 1H), 4.66 (t, J=8.2 Hz, 1H), 4.08-3.91 (m, 3H), 1.17 (t, J=7.1 Hz, 3H).

### Step 3: (R)-5-phenyl-5,6-dihydrooxazolo[2,3-c][1,2,4]triazol-3(2H)-one

To a solution of ethyl (R)-2-(4-phenyloxazolidin-2-ylidene)hydrazine-1-carboxylate (1.00 eq, 650 mg, 2.61 mmol) in toluene (40 mL), was added DIEA (1.00 eq, 0.45 mL, 2.61 mmol) at room temperature in a sealed tube. After addition, the mixture was stirred at 130° C. for 12 h. LCMS indicated about 10% product conversion. Then the reaction mixture was added with water (20 mL) and extracted with EtOAc (20 mL×3). The organics were then combined and dried (Na₂SO₄) before concentration to dryness. The crude was purified with C₁₈ column and prep-TLC (DCM/MeOH=10/1) to give the product (15 mg, 0.0701 mmol, 2.69% yield) as a white solid. MS (m/z): 204.1 [M+H]⁺. ¹H NMR (400 MHz, DMSO-d₆) δ 10.78 (s, 1H), 7.52-7.24 (m, 5H), 5.52 (dd, J=8.0, 5.2 Hz, 1H), 5.36 (t, J=8.4 Hz, 1H), 4.76 (dd, J=8.6, 5.2 Hz, 1H).

### Step 4: methyl (R)-5-(2-fluoro-4-(3-oxo-5-phenyl-5,6-dihydrooxazolo[2,3-c][1,2,4]triazol-2(3H)-yl)phenoxy)-4-methylthiazole-2-carboxylate

To a solution of (R)-5-phenyl-5,6-dihydrooxazolo[2,3-c][1,2,4]triazol-3(2H)-one (1.00 eq, 20 mg, 0.0984 mmol) in 1,4-dioxane (1 mL), were added CuI (3.00 eq, 56 mg, 0.295 mmol), K₂CO₃ (3.00 eq, 41 mg, 0.295 mmol), methyl 5-(4-bromo-2-fluoro-phenoxy)-4-methyl-thiazole-2-carboxylate (1.20 eq, 41 mg, 0.118 mmol), and (1R,2R)—N,N′-Dimethyl-1,2-cyclohexanediamine (6.00 eq, 84 mg, 0.591 mmol). The reaction mixture was stirred for 60 min at 100° C. under nitrogen. The reaction mixture was concentrated to dryness and the residue was taken up in EtOAc (30 mL) and the organics were washed with water (10 mL×3). The organics were then separated and dried with Na₂SO₄ before concentration to dryness. The crude was then purified by Prep-TLC (EtOAc/PE=1/10) to give the product (10 mg, 0.0213 mmol, 21.7% yield) as a yellow oil. MS (m/z): 469.1 [M+H]⁺.

### Step 5: (R)-5-(2-fluoro-4-(3-oxo-5-phenyl-5,6-dihydrooxazolo[2,3-c][1,2,4]triazol-2(3H)-yl) phenoxy)-4-methylthiazole-2-carboxamide Formate

A solution of (R)-5-(2-fluoro-4-(3-oxo-5-phenyl-5,6-dihydrooxazolo[2,3-c][1,2,4]triazol-2(3H)-yl)phenoxy)-4-methylthiazole-2-carboxylate (1.00 eq, 10 mg, 0.0213 mmol) in 7 N NH₃ in MeOH (2.0 mL) was stirred at 80° C. for 15 min. LCMS indicated the reaction was completed. Then it was evaporated to dryness. The crude product was purified by prep-HPLC (0.1% TFA as additive) to give the product (1.0 mg, 0.00221 mmol, 10.33% yield) as a white solid. MS (m/z): 454.1 [M+H]⁺. ¹H NMR (400 MHz, DMSO-d₆) δ 8.43 (s, 2H), 8.08 (s, 1H), 7.82-7.72 (m, 2H), 7.61-7.58 (m, 1H), 7.49-7.41 (m, 4H), 7.36 (t, J=9.1 Hz, 1H), 5.69 (dd, J=8.1, 6.0 Hz, 1H), 5.48 (t, J=8.4 Hz, 1H), 4.88 (dd, J=8.7, 6.0 Hz, 1H), 2.31 (s, 3H).

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 445 Method 72 | ++++ | 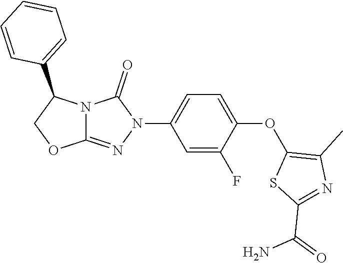 [SMILES: Cc1nc(C(N)=O)sc1Oc1ccc(-n2nc3n(c2=O)[C@H](c2ccccc2)CO3)cc1F] (R)-5-(2-fluoro-4-(3-oxo-5-phenyl-5,6-dihydrooxazolo [2,3-c][1,2,4]triazol-2(3H)-yl)phenoxy)-4-methylthiazole- 2-carboxamide | white solid, yield: 10.3% | ¹H NMR (400 MHz, DMSO-d₆) δ 8.43 (s, 2H), 8.08 (s, 1H), 7.82-7.72 (m, 2H), 7.61-7.58 (m, 1H), 7.49-7.41 (m, 4H), 7.36 (t, J = 9.1 Hz, 1H), 5.69 (dd, J = 8.1, 6.0 Hz, 1H), 5.48 (t, J = 8.4 Hz, 1H), 4.88 (dd, J = 8.7, 6.0 Hz, 1H), 2.31 (s, 3H). | 454.1 |

### Example 446: Method 73

### 2-(4-((2-(2,2-difluoro-1-hydroxyethyl)-4-methylthiazol-5-yl)oxy)-3-fluorophenyl)-4-(2,6-di fluorobenzyl)-2,4-dihydro-3H-1,2,4-triazol-3-one

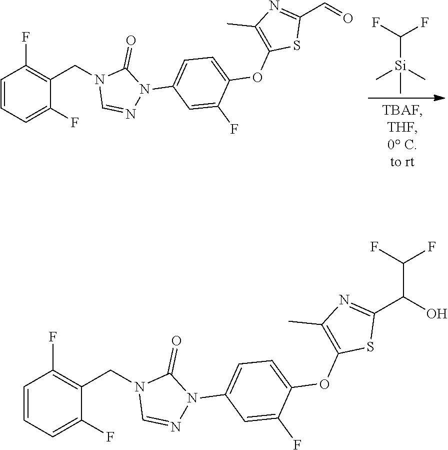

To a solution of difluoromethyl(trimethyl)silane (2.00 eq, 278 mg, 2.24 mmol) and 5-(4-(4-(2,6-difluorobenzyl)-5-oxo-4,5-dihydro-1H-1,2,4-triazol-1-yl)-2-fluorophenoxy)-4-methylthiazole-2-carbaldehyde (1.00 eq, 500 mg, 1.12 mmol) in THF (10 mL), was added TBAF (0.200 eq, 0.22 mL, 0.224 mmol) in THF (0.8 mL) at 0° C. After addition, the mixture was stirred at 0° C. for 2 h. And the mixture was then stirred at 25° C. for 12 h. LCMS indicated about 30% product conversion. Then the reaction mixture was added with water (20 mL) and extracted with EtOAc (20 mL×3). The organics were then combined and dried (with Na₂SO₄) before concentration to dryness. The crude was purified with silica gel column (PE/EtOAc=1/1) to give the product (75 mg, 0.120 mmol, 10.75% yield) as a white solid. MS (m/z): 499.1 [M+H]⁺. ¹H NMR (400 MHz, Methanol-d₄) δ 7.91 (s, 1H), 7.78 (dd, J=12.4, 2.5 Hz, 1H), 7.62 (dt, J=9.0, 2.0 Hz, 1H), 7.39-7.31 (m, 1H), 7.04 (t, J=9.0 Hz, 1H), 6.96 (t, J=8.1 Hz, 2H), 6.07-5.79 (m, 1H), 4.93 (s, 2H), 4.87-4.80 (m, 1H), 2.83 (d, J=52.8 Hz, 1H), 2.18 (s, 3H).

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 446 Method 73 | ++++ | 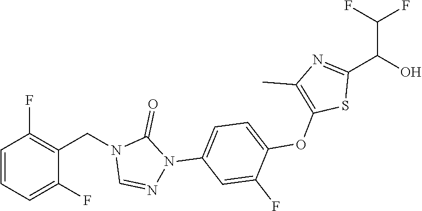 [SMILES: Cc1nc(C(O)C(F)F)sc1Oc1ccc(-n2ncn(Cc3c(F)cccc3F)c2=O)cc1F] 2-(4-((2-(2,2-difluoro-1-hydroxyethyl)-4-methylthiazol- 5-yl)oxy)-3-fluorophenyl)-4-(2,6-difluorobenzyl)- 2,4-dihydro-3H-1,2,4-triazol-3-one | white solid, yield: 10.75% | ¹H NMR (400 MHz, Methanol-d₄) δ 7.91 (s, 1H), 7.78 (dd, J = 12.4, 2.5 Hz, 1H), 7.62 (dt, J = 9.0, 2.0 Hz, 1H), 7.39-7.31 (m, 1H), 7.04 (t, J = 9.0 Hz, 1H), 6.96 (t, J = 8.1 Hz, 2H), 6.07- 5.79 (m, 1H), 4.93 (s, 2H), 4.87-4.80 (m, 1H), 2.83 (d, J = 52.8 Hz, 1H), 2.18 (s, 3H). | 499.1 |

### Example 447: Method 74

### 2-(4-((2-(1-amino-2,2-difluoroethyl)-4-methylthiazol-5-yl)oxy)-3-fluorophenyl)-4-(2,6-difluorobenzyl)-2,4-dihydro-3H-1,2,4-triazol-3-one

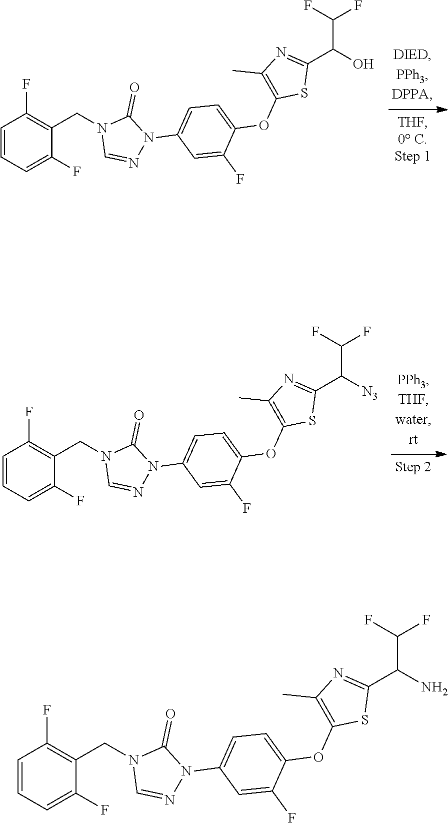 [SMILES: Cc1nc(C(O)C(F)F)sc1Oc1ccc(-n2ncn(Cc3c(F)cccc3F)c2=O)cc1F ; Cc1nc(C(N=[N+]=[N-])C(F)F)sc1Oc1ccc(-n2ncn(Cc3c(F)cccc3F)c2=O)cc1F ; Cc1nc(C(N)C(F)F)sc1Oc1ccc(-n2ncn(Cc3c(F)cccc3F)c2=O)cc1F]

### Step 1: Step 1: 2-(4-((2-(1-azido-2,2-difluoroethyl)-4-methylthiazol-5-yl)oxy)-3-fluorophenyl)-4-(2,6-difluorobenzyl)-2,4-dihydro-3H-1,2,4-triazol-3-one

To a solution of 2-(4-((2-(2,2-difluoro-1-hydroxyethyl)-4-methylthiazol-5-yl)oxy)-3-fluorophenyl)-4-(2,6-difluorobenzyl)-2,4-dihydro-3H-1,2,4-triazol-3-one (1.00 eq, 30 mg, 0.06 mmol), Triphenylphosphine (1.20 eq, 19 mg, 0.07 mmol) and DIAD (1.20 eq, 14 mg, 0.07 mmol) in THF (2 mL), was added DPPA (1.20 eq, 20 mg, 0.07 mmol) at 0° C. under N₂. After addition, the mixture was stirred at 0° C. for 2 h. LCMS indicated the reaction was completed. Then added water (20 mL) and extracted with EtOAc (20 mL×3). The organics were then combined and dried (Na₂SO₄) before concentration to dryness. The crude was purified with Prep-TLC (PE/EtOAc=1/1) to give the product (10 mg, 0.0191 mmol, 32.32% yield) as a white solid. MS (m/z): 524.1 [M+H]⁺.

### Step 2: 2-(4-((2-(1-amino-2,2-difluoroethyl)-4-methylthiazol-5-yl)oxy)-3-fluorophenyl)-4-(2,6-difluorobenzyl)-2,4-dihydro-3H-1,2,4-triazol-3-one formate

To a solution of 2-(4-((2-(1-azido-2,2-difluoroethyl)-4-methylthiazol-5-yl)oxy)-3-fluorophenyl)-4-(2,6-difluorobenzyl)-2,4-dihydro-3H-1,2,4-triazol-3-one (1.00 eq, 50 mg, 0.0955 mmol) in THF (3 mL) and water (0.6 mL), was added PPh₃ (3.00 eq, 75 mg, 0.287 mmol) at room temperature under N₂. After addition, the mixture was stirred at 25° C. for 2 h. LCMS indicated the reaction was completed. Then the reaction mixture was added with water (20 mL) and extracted with EtOAc (20 mL×3). The organics were then combined and dried (Na₂SO₄) before concentration to dryness. The crude was purified with Prep-HPLC (20%-70% ACN in water-0.1% TFA as additive) to give the product (0.9 mg, 0.002 mmol, 1.73% yield) as a white solid. MS (m/z): 498.1 [M+H]⁺. ¹H NMR (400 MHz, Methanol-d₄) δ 8.47 (s, 2H), 7.91 (s, 1H), 7.77 (dd, J=12.5, 2.6 Hz, 1H), 7.62 (dt, J=9.3, 2.0 Hz, 1H), 7.39-7.31 (m, 1H), 7.04 (t, J=9.0 Hz, 1H), 6.96 (t, J=8.1 Hz, 2H), 6.16-5.88 (m, 1H), 4.93 (s, 2H), 4.27 (s, 1H), 2.17 (s, 3H).

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Example 447 Method 74 | ++++ | 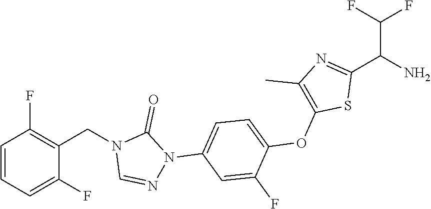 [SMILES: Cc1nc(C(N)C(F)F)sc1Oc1ccc(-n2ncn(Cc3c(F)cccc3F)c2=O)cc1F] 2-(4-((2-(1-amino-2,2-difluoroethyl)-4-methylthiazol-5-yl)oxy)- 3-fluorophenyl)-4-(2,6-difluorobenzyl)-2,4-dihydro-3H-1,2,4- triazol-3-one formate | white solid, yield: 1.73% | ¹H NMR (400 MHz, Methanol-d₄) δ 8.47 (s, 2H), 7.91 (s, 1H), 7.77 (dd, J = 12.5, 2.6 Hz, 1H), 7.62 (dt, J = 9.3, 2.0 Hz, 1H), 7.39-7.31 (m, 1H), 7.04 (t, J = 9.0 Hz, 1H), 6.96 (t, J = 8.1 Hz, 2H), 6.16-5.88 (m, 1H), 4.93 (s, 2H), 4.27 (s, 1H), 2.17 (s, 3H). | 498.1 |

### Example 448: Method 75

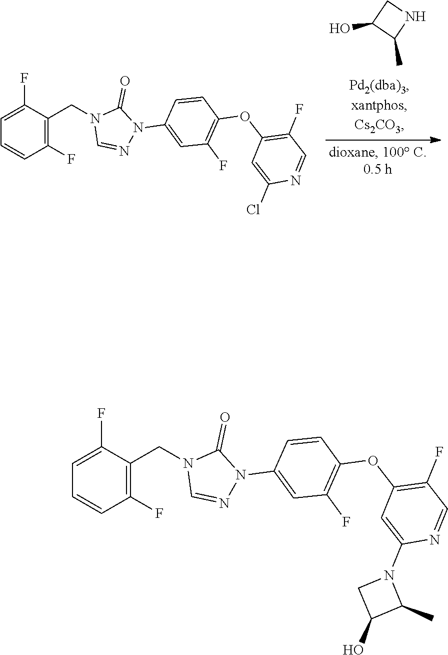

To a solution of 2-[4-[(2-chloro-5-fluoro-4-pyridyl)oxy]-3-fluoro-phenyl]-4-[(2,6-difluorophenyl)methyl]-1,2,4-triazol-3-one (1.00 eq, 100 mg, 0.222 mmol) in 1,4-Dioxane (10 mL), were added xantphos (0.400 eq, 51 mg, 0.0887 mmol), Pd₂(dba)₃ (0.200 eq, 41 mg, 0.0444 mmol), and (2 S, 3S)-2-methylazetidin-3-ol; hydrochloride (1.50 eq, 41 mg, 0.333 mmol). The mixture was stirred for 30 mci at 100° C. under nitrogen. The reaction was concentrated to dryness and the residue was taken up in EA (20 mL). The organics were washed with 2×20 mL water and then with 1×20 mL saturated brine solution. The organics were then separated and dried (MgSO₄) before concentration to dryness. The crude was then purified by flash column chromatography eluted with 80% ACN in H₂O. The desired fractions were concentrated to dryness in vacuo to afford 4-[(2,6-difluorophenyl)methyl]-2-[3-fluoro-4-[[5-fluoro-2-[(2S,3S)-3-hydroxy-2-methyl-azetidin-1-yl]-4-pyridyl]oxy]phenyl]-1,2,4-triazol-3-one (2.7 mg, 0.00431 mmol, 1.94 yield) as a white solid. MS (m/z): 502.2 [M+H]⁺. ¹H NMR (400 MHz, Chloroform-d) δ 8.08 (d, J=12.8 Hz, 1H), 7.98-7.94 (m, 1H), 7.81 (dd, J=12.4, 7.6 Hz, 1H), 7.64 (s, 1H), 7.49-7.33 (m, 1H), 7.31-7.23 (m, 2H), 7.00 (t, J=8.0 Hz, 2H), 5.49 (d, J=6.0 Hz, 1H), 5.00 (s, 2H), 4.87-4.66 (m, 2H), 4.50-4.39 (m, 1H), 4.28-4.15 (m, 1H), 1.26 (s, 3H).

| Compound | | | | | MS |
|---|---|---|---|---|---|
| No. | | | Appearance | | (m/z) |
| Method | Activity | Structure and Name | and Yield | ¹H NMR Data | [M + H]⁺ |
| Method 75 Example 448 | ++++ | 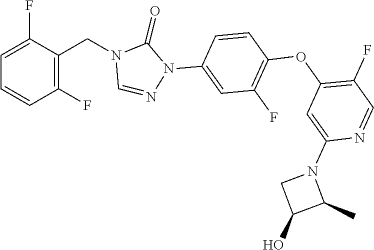 [SMILES: C[C@H]1[C@@H](O)CN1c1cc(Oc2ccc(-n3ncn(Cc4c(F)cccc4F)c3=O)cc2F)c(F)cn1] 4-(2,6-difluorobenzyl)-2-(3-fluoro-4-((5-fluoro-2-((2S,3S)-3- hydroxy-2-methylazetidin-1-yl)pyridin-4-yl)oxy)phenyl)-2,4- dihydro-3H-1,2,4-triazol-3-one | white solid, yield: 1.94% | ¹H NMR (400 MHz, Chloroform-d) δ 8.08 (d, J = 12.8 Hz, 1H), 7.98-7.94 (m, 1H), 7.81 (dd, J = 12.4, 7.6 Hz, 1H), 7.64 (s, 1H), 7.49-7.33 (m, 1H), 7.31-7.23 (m, 2H), 7.00 (t, J = 8.0 Hz, 2H), 5.49 (d, J = 6.0 Hz, 1H), 5.00 (s, 2H), 4.87-4.66 (m, 2H), 4.50-4.39 (m, 1H), 4.28- 4.15 (m, 1H), 1.26 (s, 3H). | 502.2 |

### Example 449: Method 76

### 2-(4-((2-acetyl-4-methylthiazol-5-yl)oxy)-3-fluorophenyl)-4-(2,6-difluorobenzyl)-2,4-dihydro-3H-1,2,4-triazol-3-one

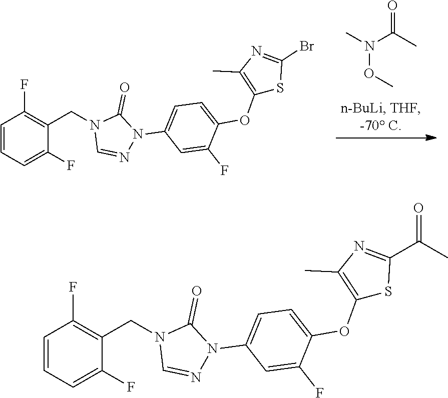

To a solution of 2-[4-(2-bromo-4-methyl-thiazol-5-yl)oxy-3-fluoro-phenyl]-4-[(2,6-difluorophenyl)methyl]-1,2,4-triazol-3-one (1.00 eq, 500 mg, 1.01 mmol) and N-methoxy-N-methyl-acetamide (1.20 eq, 124 mg, 1.21 mmol) in THF (20 mL), was added n-BuLi (2.20 eq, 1.0 mL, 2.21 mmol), at −70° C. under N₂. After addition, the mixture was stirred at −70° C. for 1 h. LCMS indicated the reaction was completed. Then the reaction mixture was added with water (20 mL) and extracted with EtOAc (20 mL×3). The organics were then combined and dried (Na₂SO₄) before concentration to dryness. The crude was purified with silica gel column (PE/EtOAc=1/1) to give the product (220 mg, 0.463 mmol, 46.10% yield) as a white solid. MS (m/z): 461.1 [M+H]⁺. ¹H NMR (400 MHz, DMSO-d₆) δ 8.37 (s, 1H), 7.92 (dd, J=12.5, 2.6 Hz, 1H), 7.76-7.71 (m, 1H), 7.56-7.40 (m, 2H), 7.16 (t, J=8.1 Hz, 2H), 4.98 (s, 2H), 2.55 (s, 3H), 2.36 (s, 3H).

| Com- | | | Appear- | | MS |
|---|---|---|---|---|---|
| pound | | | ance | | (m/z) |
| No. | Ac- | | and | | [M + |
| Method | tivity | Structure and Name | Yield | ¹H NMR Data | H]⁺ |
| Ex- am- ple 449 Method 76 | ++++ | 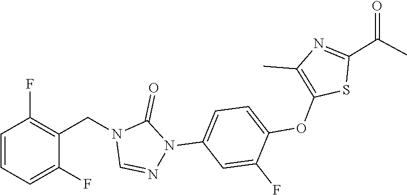 [SMILES: CC(=O)c1nc(C)c(Oc2ccc(-n3ncn(Cc4c(F)cccc4F)c3=O)cc2F)s1] | white solid, yield: 46.10% | ¹H NMR (400 MHz, DMSO-d₆) δ 8.37 (s, 1H), 7.92 (dd, J = 12.5, 2.6 Hz, 1H), 7.76- 7.71 (m, 1H), 7.56-7.40 (m, 2H), 7.16 (t, J = 8.1 Hz, 2H), 4.98 (s, 2H), 2.55 (s, 3H), 2.36 (s, 3H). | 461.1 |
| | | 2-(4-((2-acetyl-4-methylthiazol- | | | |
| | | 5-yl)oxy)-3-fluorophenyl)-4- | | | |
| | | (2,6-difluorobenzyl)-2,4-dihydro- | | | |
| | | 3H-1,2,4-triazol-3-one | | | |

### Example 450: Method 77

### 2-(4-((2-(3-amino-3-(difluoromethyl)azetidin-1-yl)-4-methylthiazol-5-yl)oxy)-3-fluorophen yl)-4-(2,6-difluorobenzyl)-2,4-dihydro-3H-1,2,4-triazol-3-one

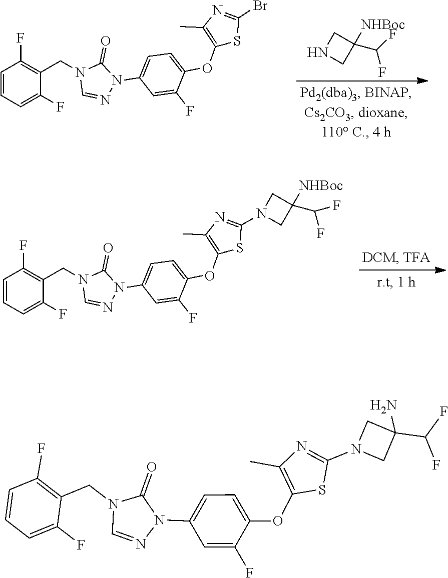 [SMILES: Cc1nc(Br)sc1Oc1ccc(-n2ncn(Cc3c(F)cccc3F)c2=O)cc1F ; CC(C)(C)OC(=O)NC1(C(F)F)CNC1 ; Cc1nc(N2CC(NC(=O)OC(C)(C)C)(C(F)F)C2)sc1Oc1ccc(-n2ncn(Cc3c(F)cccc3F)c2=O)cc1F ; Cc1nc(N2CC(N)(C(F)F)C2)sc1Oc1ccc(-n2ncn(Cc3c(F)cccc3F)c2=O)cc1F]

Step 1: To a solution of 2-(4-((2-bromo-4-methylthiazol-5-yl)oxy)-3-fluorophenyl)-4-(2,6-difluorobenzyl)-2,4-dihydro-3H-1,2,4-triazol-3-one (1.00 eq, 300 mg, 0.603 mmol), tert-butyl (3-(difluoromethyl)azetidin-3-yl)carbamate hydrochloride (1.50 eq, 234 mg, 0.905 mmol) and Cs₂CO₃ (2.00 eq, 393 mg, 1.21 mmol) in toluene (5 mL), was added Tris(dibenzylideneacetonyl)bis-palladium (0.150 eq, 83 mg, 0.0905 mmol) under N₂ at room temperature. After addition, the mixture was heated to 110° C. and stirred for 3 h. Then the mixture was added with water (10 mL) and extracted with EtOAc (10 mL×3). The organics were then combined and dried (Na₂SO₄) before concentration to dryness. The crude was purified with silica gel column (PE/EtOAc=1/1) to give the product (140 mg, 0.208 mmol, 34.52% yield) as a white solid. MS (m/z): 621.2 [M+H]⁺.

Step 2: To a solution of tert-butyl N-[3-(difluoromethyl)-1-[5-[4-[4-[(2,6-difluorophenyl)methyl]-5-oxo-1,2,4-triazol-1-yl]-2-fluoro-phenoxy]-4-methyl-thiazol-2-yl]azetidin-3-yl]carbamate (1.00 eq, 120 mg, 0.179 mmol) in DCM (1 mL), was added TFA (50.0 eq, 0.68 mL, 8.93 mmol) at room temperature. After addition, the mixture was stirred at 25° C. for 2 h. LCMS indicated the reaction was completed. The reaction mixture was concentrated under reduced pressure and purified with Prep-HPLC to give the product (50 mg, 0.0755 mmol, 42.28% yield) as a white solid. MS (m/z): 539.1 [M+H]⁺. ¹H NMR (400 MHz, DMSO-d₆) δ 8.32 (s, 1H), 7.83 (dd, J=12.6, 2.5 Hz, 1H), 7.67-7.63 (m, 1H), 7.52-7.45 (m, 1H), 7.19-7.12 (m, 3H), 6.19-5.92 (m, 1H), 4.97 (s, 2H), 4.00 (d, J=8.4 Hz, 2H), 3.72 (d, J=8.4 Hz, 2H), 2.02 (s, 3H).

The synthesis procedure of Examples (Compounds) 451-458 were similar to the procedure of Example 450.

| Com- | | | Ap- | | |
|---|---|---|---|---|---|
| pound | | | pear- | | MS |
| No. | | | ance | | (m/z) |
| Meth- | Ac- | | and | | [M + |
| od | tivity | Structure and Name | Yield | ¹H NMR Data | H]⁺ |
| Ex- am- ple 450 Meth- od 77 | ++++ | 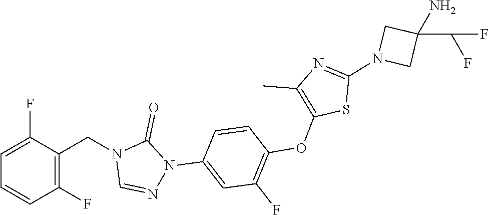 [SMILES: Cc1nc(N2CC(N)(C(F)F)C2)sc1Oc1ccc(-n2ncn(Cc3c(F)cccc3F)c2=O)cc1F] | white solid, yield: 42.28% | ¹H NMR (400 MHz, DMSO-d₆) δ 8.32 (s, 1H), 7.83 (dd, J = 12.6, 2.5 Hz, 1H), 7.67- 7.63 (m, 1H), 7.52-7.45 (m, 1H), 7.19-7.12 (m, 3H), 6.19-5.92 (m, 1H), 4.97 (s, 2H), 4.00 (d, J = 8.4 Hz, 2H), 3.72 (d, J = 8.4 Hz, 2H), 2.02 (s, 3H). | 539.1 |
| | | 2-(4-((2-(3-amino-3-(difluoromethyl) | | | |
| | | azetidin-1-yl)-4-methylthiazol- | | | |
| | | 5-yl)oxy)-3-fluorophenyl)-4-(2,6- | | | |
| | | difluorobenzyl)-2,4-dihydro-3H- | | | |
| | | 1,2,4-triazol-3-one | | | |
| Ex- am- ple 451 Meth- od 77 | ++++ | 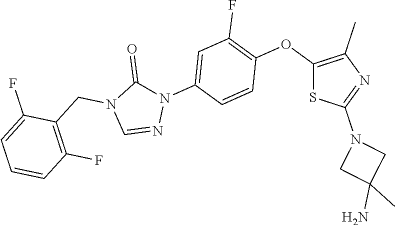 [SMILES: Cc1nc(N2CC(C)(N)C2)sc1Oc1ccc(-n2ncn(Cc3c(F)cccc3F)c2=O)cc1F] | yellow solid, yield: 32.69% | ¹H NMR (400 MHz, DMSO-d₆) δ 8.35 (d, J = 10.6 Hz, 3H), 7.84 (dd, J = 12.7, 2.6 Hz, 1H), 7.69-7.64 (m, 1H), 7.52-7.43 (m, 2H), 7.19-7.11 (m, 3H), 4.97 (s, 2H), 4.04 (d, J = 8.8 Hz, 2H), 3.93 (d, J = 8.8 Hz, 2H), 2.04 (s, 3H), 1.57 (s, 3H). | 503.1 |
| | | 2-(4-((2-(3-amino-3-methylazetidin- | | | |
| | | 1-yl)-4-methylthiazol-5-yl)oxy)- | | | |
| | | 3-fluorophenyl)-4-(2,6-difluoro | | | |
| | | benzyl)-2,4-dihydro-3H-1,2,4- | | | |
| | | triazol-3-one | | | |
| Ex- am- ple 452 Meth- od 77 | ++++ | 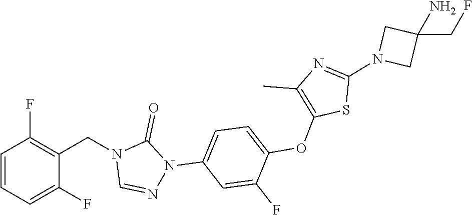 [SMILES: Cc1nc(N2CC(N)(CF)C2)sc1Oc1ccc(-n2ncn(Cc3c(F)cccc3F)c2=O)cc1F] | white solid, yield: 61.17% | NA | 521.1 |
| | | 2-(4-((2-(3-amino-3-(fluoromethyl) | | | |
| | | azetidin-1-yl)-4-methylthiazol-5- | | | |
| | | yl)oxy)-3-fluorophenyl)-4-(2,6- | | | |
| | | difluorobenzyl)-2,4-dihydro-3H- | | | |
| | | 1,2,4-triazol-3-one | | | |
| Ex- am- ple 453 Meth- od 77 | ++++ | 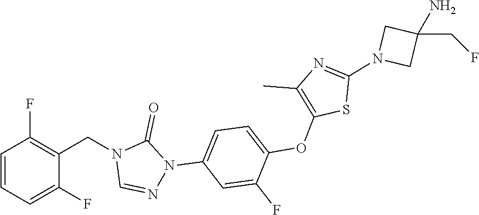 [SMILES: Cc1nc(N2CC(N)(CF)C2)sc1Oc1ccc(-n2ncn(Cc3c(F)cccc3F)c2=O)cc1F] | white solid, yield: 25.55% | ¹H NMR (400 MHz, Methanol-d₄) δ 8.01 (s, 1H), 7.69 (d, J = 9.7 Hz, 2H), 7.43 (d, J = 8.4 Hz, 1H), 7.27 (d, J = 8.2 Hz, 1H), 7.05 (t, J = 8.1 Hz, 2H), 5.03 (s, 2H), 4.34 (s, 1H), 4.26 (s, 1H), 4.19 (d, J = 9.5 Hz, 2H), 4.10 (d, J = 9.2 Hz, 2H), 3.99 (s, 2H), 2.24 (s, 3H). | 519.2 |
| | | 2-(4-((2-(3-amino-3-(fluoromethyl) | | | |
| | | azetidin-1-yl)-4-methylthiazol-5- | | | |
| | | yl)methyl)-3-fluorophenyl)-4-(2,6- | | | |
| | | difluorobenzyl)-2,4-dihydro-3H- | | | |
| | | 1,2,4-triazol-3-one | | | |
| Ex- am- ple 454 Meth- od 77 | ++++ | 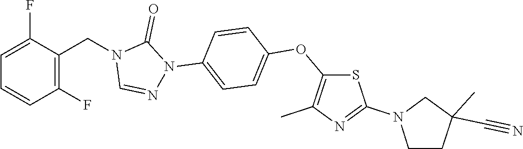 [SMILES: Cc1nc(N2CCC(C)(C#N)C2)sc1Oc1ccc(-n2ncn(Cc3c(F)cccc3F)c2=O)cc1] | White solid, yield: 56.4% | ¹H NMR (400 MHz, Chloroform-d) δ 7.91-7.83 (m, 2H), 7.55 (s, 1H), 7.36 (tt, J = 8.4, 6.4 Hz, 1H), 7.07- 7.02 (m, 2H), 7.01- 6.94 (m, 2H), 4.97 (d, J = 1.2 Hz, 2H), 3.95 (d, J = | 509.2 |
| | | | | 10.4 Hz, 1H), 3.72- | |
| | | 1-(5-(4-(4-(2,6-difluorobenzyl)-5- | | 3.62 (m, 1H), | |
| | | oxo-4,5-dihydro-1H-1,2,4-triazol- | | 3.62-3.54 (m, | |
| | | 1-yl)phenoxy)-4-methylthiazol-2- | | 1H), 3.42 (d, J = | |
| | | yl)-3-methylpyrrolidine-3- | | 10.4 Hz, 1H), 2.58- | |
| | | carbonitrile | | 2.47 (m, 1H), | |
| | | | | 2.19-2.01 (m, | |
| | | | | 4H), 1.57 (s, 3H). | |
| Ex- am- ple 455 Meth- od 77 | ++++ | 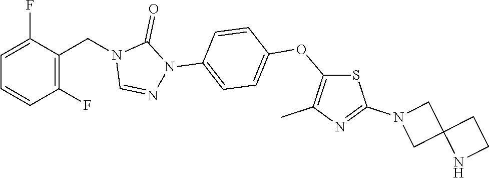 [SMILES: Cc1nc(N2CC3(CCN3)C2)sc1Oc1ccc(-n2ncn(Cc3c(F)cccc3F)c2=O)cc1] | light yellow solid, yield: 70.2% | ¹H NMR (400 MHz, Chloroform-d) δ 10.56 (brs, 1H), 7.90 (d, J = 8.4 Hz, 2H), 7.57 (s, 1H), 7.34 (tt, J = 8.4, 6.4 Hz, 1H), 7.04 (d, J = 8.4 Hz, 2H), 6.95 (t, J = 8.0 Hz, 2H), 5.64 (brs, 2H), 4.94 | 497.2 |
| | | | | (brs, 2H), 4.77 | |
| | | 4-(2,6-difluorobenzyl)-2-(4-((4- | | (brs, 2H), 4.13 | |
| | | methyl-2-(1,6-diazaspiro[3.3]heptan- | | (brs, 2H), 3.00 (s, | |
| | | 6-yl)thiazol-5-yl)oxy)phenyl)-2,4- | | 2H), 2.20 (s, 3H). | |
| | | dihydro-3H-1,2,4-triazol-3-one | | | |
| Ex- am- ple 456 Meth- od 77 | ++++ | 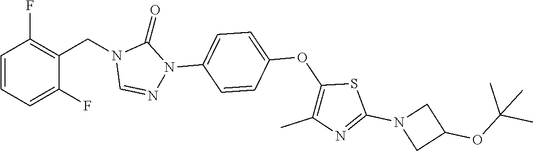 [SMILES: Cc1nc(N2CC(OC(C)(C)C)C2)sc1Oc1ccc(-n2ncn(Cc3c(F)cccc3F)c2=O)cc1] | light yellow solid, yield: 28.5% | ¹H NMR (400 MHz, Chloroform-d) δ 7.91-7.82 (m, 2H), 7.56 (s, 1H), 7.45-7.30 (m, 1H), 7.06-6.94 (m, 4H), 4.97 (d, J = 1.2 Hz, 2H), 4.64 (tt, J = 6.4, 5.2 Hz, 1H), 4.28-4.24 | 528.2 |
| | | | | (m, 2H), 3.97- | |
| | | 2-(4-((2-(3-(tert-butoxy)azetidin- | | 3.89 (m, 2H), 2.10 | |
| | | 1-yl)-4-methylthiazol-5-yl)oxy) | | (s, 3H), 1.19 (s, 9H). | |
| | | phenyl)-4-(2,6-difluorobenzyl)-2,4- | | | |
| | | dihydro-3H-1,2,4-triazol-3-one | | | |
| Ex- am- ple 457 Meth- od 77 | ++++ | 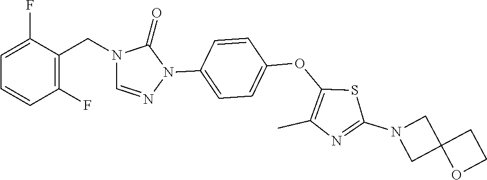 [SMILES: Cc1nc(N2CC3(CCO3)C2)sc1Oc1ccc(-n2ncn(Cc3c(F)cccc3F)c2=O)cc1] | light yellow yield: 18.0% | ¹H NMR (400 MHz, Chloroform-d) δ 7.90 (d, J = 9.2 Hz, 2H), 7.56 (s, 1H), 7.39-7.32 (m, 1H), 7.05- 6.94 (m, 4H), 4.97 (d, J = 1.6 Hz, 2H), 4.56 (t, J = 7.4 Hz, 2H), 4.40 (brs, 4H), 2.93 (t, J = 7.2 Hz, 2H), 2.17 (s, 3H). | 498.2 |
| | | 4-(2,6-difluorobenzyl)-2-(4-((4- | | | |
| | | methyl-2-(1-oxa-6-azaspiro[3.3] | | | |
| | | heptan-6-yl)thiazol-5-yl)oxy)phenyl)- | | | |
| | | 2,4-dihydro-3H-1,2,4-triazol-3-one | | | |
| Ex- am- ple 458 Meth- od 77: | ++++ | 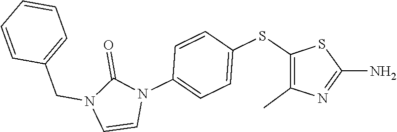 [SMILES: Cc1nc(N)sc1Sc1ccc(-n2ccn(Cc3ccccc3)c2=O)cc1] | off- white solid, yield: 69.6% | NA | 396.1 |
| | | 2-(4-((2-amino-4-methylthiazol-5- | | | |
| | | yl)thio)phenyl)-4-benzyl-2,4- | | | |
| | | dihydro-3H-1,2,4-triazol-3-one | | | |

### Example 459: Method 78

### 4-(2,6-difluorobenzyl)-2-(4-((2-(1-hydroxyethyl)-4-methylthiazol-5-yl)oxy)phenyl)-2,4-dihydro-3H-1,2,4-triazol-3-one

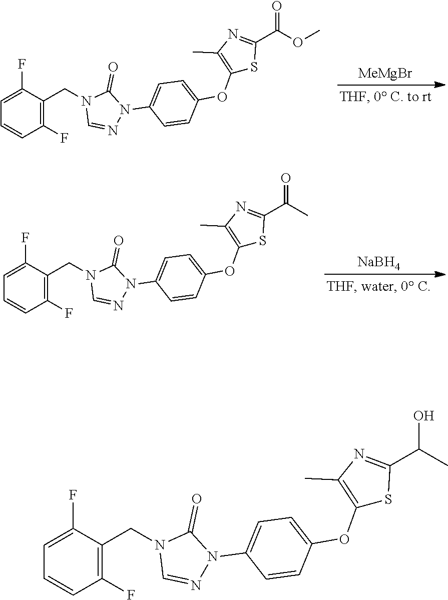 [SMILES: "MeMgBr" ; COC(=O)c1nc(C)c(Oc2ccc(-n3ncn(Cc4c(F)cccc4F)c3=O)cc2)s1 ; CC(=O)c1nc(C)c(Oc2ccc(-n3ncn(Cc4c(F)cccc4F)c3=O)cc2)s1 ; "NaBH4" ; Cc1nc(C(C)O)sc1Oc1ccc(-n2ncn(Cc3c(F)cccc3F)c2=O)cc1]

Step 1: To a solution of methyl 5-(4-(4-(2,6-difluorobenzyl)-5-oxo-4,5-dihydro-1H-1,2,4-triazol-1-yl)phenoxy)-4-methylthiazole-2-carboxylate (1.00 eq, 100 mg, 0.218 mmol) in THF (2 mL), was added Methylmagnesium bromide (1.00 eq, 0.21 mL, 0.218 mmol) at 0° C. under N₂. After addition, the mixture was stirred at 25° C. for 5 h. Then the mixture was added with water (20 mL) and extracted with EtOAc (20 mL×3). The organics were then combined and dried (Na₂SO₄) before concentration to dryness. The crude was purified with Prep-TLC (PE/EtOAc=1/1) to give the product 2-[4-(2-acetyl-4-methyl-thiazol-5-yl)oxyphenyl]-4-[(2,6-difluorophenyl)methyl]-1,2,4-triazol-3-one (20 mg, 0.0407 mmol, 18.65% yield) as a white solid. MS (m/z): 443.1 [M+H]⁺.

### Step 2: 4-(2,6-difluorobenzyl)-2-(4-((2-(1-hydroxyethyl)-4-methylthiazol-5-yl)oxy)phenyl)-2,4-dihydro-3H-1,2,4-triazol-3-one

To a solution of 2-(4-((2-acetyl-4-methylthiazol-5-yl)oxy)phenyl)-4-(2,6-difluorobenzyl)-2,4-dihydro-3H-1,2,4-triazol-3-one (1.00 eq, 20 mg, 0.045 mmol) in THF (1 mL) and water (0.5 mL), was added NaBH₄ (3.00 eq, 5 mg, 0.135 mmol) at 0° C. under N₂. After addition, the mixture was stirred at 0° C. for 1 h. LCMS indicated the reaction was completed. Then the mixture was added with water (2 mL) and extracted with EtOAc (5 mL×3). The organics were then combined and dried (Na₂SO₄) before concentration to dryness. The crude was purified with silica gel column (PE EtOAc=1/1) to give the racemic product (4.0 mg, 7.880 yield) as a white solid. MS (m/z): 445.1 [M+H]⁺. H NMR (400 MHz, DMSO-d₆) δ 8.28 (s, 1H), 7.91-7.81 (m, 2H), 7.52-7.44 (m, 1H), 7.25-7.08 (m, 4H), 6.08 (d, J=5.2 Hz, 1H), 4.97 (s, 2H), 4.82-4.74 (m, 1H), 2.14 (s, 3H), 1.43 (d, J=6.4 Hz, 3H). The racemic product was purified by chiral T-PLC to give two products.

| Compound | | | Appear- | | |
|---|---|---|---|---|---|
| No. | Ac- | | ance | | MS(m/z) |
| Method | tivity | Structure and Name | and Yield | ¹H NMR Data | [M + H]⁺ |
| Example 459 Method 78 | ++++ | 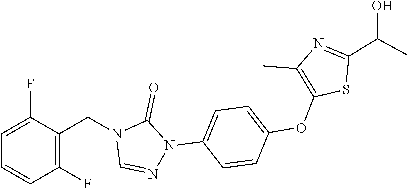 [SMILES: Cc1nc(C(C)O)sc1Oc1ccc(-n2ncn(Cc3c(F)cccc3F)c2=O)cc1] | white solid, yield: 7.88% | ¹H NMR (400 MHz, DMSO-d₆) δ 8.28 (s, 1H), 7.91-7.81 (m, 2H), 7.52-7.44 (m, 1H), 7.25-7.08 (m, 4H), 6.08 (d, J = 5.2 Hz, 1H), 4.97 (s, 2H), 4.82-4.74 (m, 1H), 2.14 (s, 3H), 1.43 (d, J = 6.4 Hz, 3H). | 445.1 |
| | | 4-(2,6-difluorobenzyl)-2-(4-((2- | | | |
| | | (1-hydroxyethyl)-4-methylthiazol- | | | |
| | | 5-yl)oxy)phenyl)-2,4-dihydro- | | | |
| | | 3H-1,2,4-triazol-3-one | | | |
| Example 460 Method 78 | ++++ | 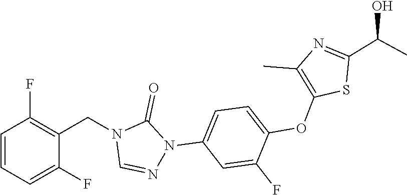 [SMILES: Cc1nc([C@H](C)O)sc1Oc1ccc(-n2ncn(Cc3c(F)cccc3F)c2=O)cc1F] | yellow solid, yield: 20.56% | ¹H NMR (400 MHz, DMSO-d₆) δ 8.34 (s, 1H), 7.87 (dd, J = 12.6, 2.5 Hz, 1H), 7.69-7.65 (m, 1H), 7.52-7.45 (m, 1H), 7.22-7.12 (m, 3H), 6.09 (d, J = 5.2 Hz, 1H), 4.97 (s, 2H), 4.84-4.68 (m, 1H), 2.18 (s, 3H), 1.41 (d, J = 6.5 Hz, 3H). | 463.1 |
| | | (S)-4-(2,6-difluorobenzyl)-2-(3- | | | |
| | | fluoro-4-((2-(1-hydroxyethyl)-4- | | | |
| | | methylthiazol-5-yl)oxy)phenyl)- | | | |
| | | 2,4-dihydro-3H-1,2,4-triazol-3- | | | |
| | | one | | | |
| | | Unknown absolute | | | |
| | | configuration | | | |
| Example 461 Method 78 | ++++ | 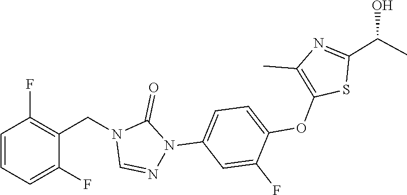 [SMILES: Cc1nc([C@@H](C)O)sc1Oc1ccc(-n2ncn(Cc3c(F)cccc3F)c2=O)cc1F] | yellow solid, yield: 20.29% | ¹H NMR (400 MHz, DMSO-d₆) δ 8.34 (s, 1H), 7.87 (dd, J = 12.6, 2.5 Hz, 1H), 7.69-7.65 (m, 1H), 7.52-7.45 (m, 1H), 7.22-7.12 (m, 3H), 6.09 (d, J = 5.2 Hz, 1H), 4.97 (s, 2H), 4.84-4.68 (m, 1H), 2.18 (s, 3H), 1.41 (d, J = 6.5 Hz, 3H). | 463.1 |
| | | (R)-4-(2,6-difluorobenzyl)-2-(3- | | | |
| | | fluoro-4-((2-(1-hydroxyethyl)-4- | | | |
| | | methylthiazol-5-yl)oxy)phenyl)- | | | |
| | | 2,4-dihydro-3H-1,2,4-triazol-3- | | | |
| | | one | | | |
| | | Unknown absolute | | | |
| | | configuration | | | |

### Example 462: Method 79

### 4-(2,6-difluorobenzyl)-2-(3-fluoro-4-((2-(2-hydroxypropan-2-yl)-4-methylthiazol-5-yl)oxy)phenyl)-2,4-dihydro-3H-1,2,4-triazol-3-one

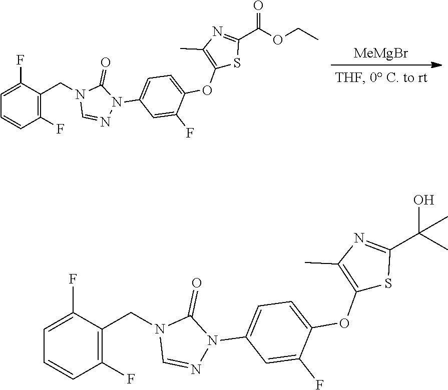 [SMILES: "MeMgBr" ; CCOC(=O)c1nc(C)c(Oc2ccc(-n3ncn(Cc4c(F)cccc4F)c3=O)cc2F)s1 ; Cc1nc(C(C)(C)O)sc1Oc1ccc(-n2ncn(Cc3c(F)cccc3F)c2=O)cc1F]

To a solution of methyl ethyl 5-(4-(4-(2,6-difluorobenzyl)-5-oxo-4,5-dihydro-1H-1,2,4-triazol-1-yl)-2-fluorophenoxy)-4-met hylthiazole-2-carboxylate (1.00 eq, 200 mg, 0.420 mmol) in THF (10 mL), was added dropwise MeMgBr (2.20 eq, 0.93 mL, 0.924 mmol) at 0° C. After addition, the mixture was stirred at room temperature for 1 h. TLC indicated the reaction was completed. Then the mixture was added with saturated NH₄Cl aq. (20 mL) and extracted with EtOAc (20 mL×3). The organics were then combined and dried (Na₂SO₄) before concentration to dryness. The crude was purified with Prep-TLC (PE/EtOAc=1/1) to give the product (121 mg, 0.248 mmol, 59.04% yield) as a white solid. MS (m/z): 477.1 [M+H]⁺. ¹H NMR (400 MHz, DMSO-d₆) δ 8.35 (s, 1H), 7.87 (dd, J=12.6, 2.5 Hz, 1H), 7.69-7.65 (m, 1H), 7.52-7.44 (m, 1H), 7.22-7.13 (m, 3H), 5.96 (s, 1H), 4.98 (s, 2H), 2.18 (s, 3H), 1.46 (s, 6H).

The synthesis procedure of Examples (Compounds) 463-473 were similar to the procedure of Example 462.

| | | | | | MS |
|---|---|---|---|---|---|
| Compound | | | Appear- | | (m/z) |
| No. | | | ance | | [M + |
| Method | Activity | Structure and Name | and Yield | ¹H NMR Data | H]⁺ |
| Example 462 Method 79 | ++++ | 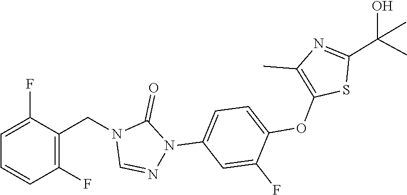 [SMILES: Cc1nc(C(C)(C)O)sc1Oc1ccc(-n2ncn(Cc3c(F)cccc3F)c2=O)cc1F] | white solid, yield: 59.04% | ¹H NMR (400 MHz, DMSO-d₆) δ 8.35 (s, 1H), 7.87 (dd, J = 12.6, 2.5 Hz, 1H), 7.69-7.65 (m, 1H), 7.52-7.44 (m, 1H), 7.22-7.13 (m, 3H), 5.96 (s, 1H), 4.98 (s, 2H), 2.18 (s, 3H), 1.46 (s, 6H). | 477.1 |
| | | 4-(2,6-difluorobenzyl)-2-(3-fluoro- | | | |
| | | 4-((2-(2-hydroxypropan-2-yl)- | | | |
| | | 4-methylthiazol-5-yl)oxy)phenyl)- | | | |
| | | 2,4-dihydro-3H-1,2,4-triazol- | | | |
| | | 3-one | | | |
| Example 463 Method 79 | ++++ | 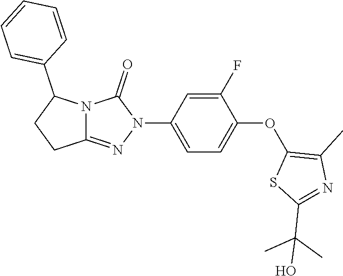 [SMILES: Cc1nc(C(C)(C)O)sc1Oc1ccc(-n2nc3n(c2=O)C(c2ccccc2)CC3)cc1F] | white solid, yield: 5.97% | ¹H NMR (400 MHz, DMSO-d₆) δ 7.84 (dd, J = 12.8, 2.4 Hz, 1H), 7.70-7.62 (m, 1H), 7.43-7.36 (m, 2H), 7.36-7.29 (m, 3H), 7.20 (t, J = 9.2 Hz, 1H), 5.94 (s, 1H), 5.31 (dd, J = 8.0, 4.8 Hz, 1H), 3.09-2.98 (m, 2H), 2.97- 2.85 (m, 1H), 2.39-2.27 (m, 1H), 2.19 (s, 3H), 1.46 (s, 6H). | 467.2 |
| | | 2-(3-fluoro-4-((2-(2-hydroxypro- | | | |
| | | pan-2-yl)-4-methylthiazol-5-yl) | | | |
| | | oxy)phenyl)-5-phenyl-2,5,6,7- | | | |
| | | tetrahydro-3H-pyrrolo[2,1-c] | | | |
| | | [1,2,4]triazol-3-one | | | |
| Example 464 Method 79 | +++ | 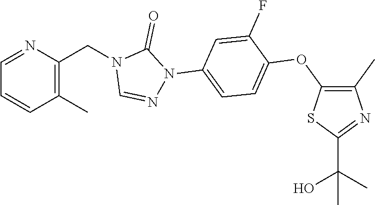 [SMILES: Cc1cccnc1Cn1cnn(-c2ccc(Oc3sc(C(C)(C)O)nc3C)c(F)c2)c1=O] | white solid, yield: 26.3% | ¹H NMR (400 MHz, DMSO-d₆) δ 8.33-8.30 (m, 1H), 8.28 (s, 1H), 7.92 (dd, J = 12.4, 2.4 Hz, 1H), 7.74 (dt, J = 9.2, 1.6 Hz, 1H), 7.64 (d, J = 7.6 Hz, 1H), 7.28- 7.19 (m, 2H), 5.94 (brs, 1H), 5.03 (s, 2H), 2.38 (s, 3H), 2.19 (s, 3H), 1.47 (s, 6H). | 456.2 |
| | | 2-(3-fluoro-4-((2-(2-hydroxypro- | | | |
| | | pan-2-yl)-4-methylthiazol-5-yl) | | | |
| | | oxy)phenyl)-4-((3-methylpyridin- | | | |
| | | 2-yl)methyl)-2,4-dihydro-3H- | | | |
| | | 1,2,4-triazol-3-one | | | |
| Example 465 Method 79 | ++++ | 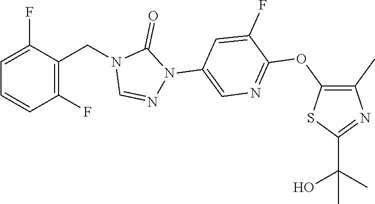 [SMILES: Cc1nc(C(C)(C)O)sc1Oc1ncc(-n2ncn(Cc3c(F)cccc3F)c2=O)cc1F] | white solid, yield: 19.8% | ¹H NMR (400 MHz, DMSO-d₆) δ 8.48 (d, J = 2.4 Hz, 1H), 8.40 (s, 1H), 8.30 (dd, J = 11.2, 2.4 Hz, 1H), 7.49 (ddd, J = 14.8, 8.4, 6.4 Hz, 1H), 7.16 (t, J = 8.0 Hz, 2H), 5.95 (brs, 1H), 4.99 (s, 2H), 2.17 (s, 3H), 1.48 (s, 6H). | 478.2 |
| | | 4-(2,6-difluorobenzyl)-2-(5-fluoro- | | | |
| | | 6-((2-(2-hydroxypropan-2-yl)- | | | |
| | | 4-methylthiazol-5-yl)oxy)pyridin- | | | |
| | | 3-yl)-2,4-dihydro-3H-1,2,4- | | | |
| | | triazol-3-one | | | |
| Example 466 Method 79 | ++++ | 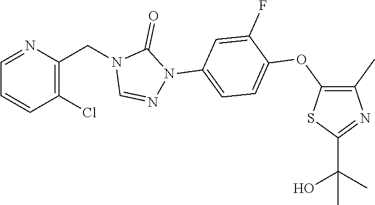 [SMILES: Cc1nc(C(C)(C)O)sc1Oc1ccc(-n2ncn(Cc3ncccc3Cl)c2=O)cc1F] | white solid, yield: 32.6% | ¹H NMR (400 MHz, DMSO-d₆) δ 8.47 (dd, J = 4.8, 1.2 Hz, 1H), 8.33 (s, 1H), 8.01 (dd, J = 8.0, 1.2 Hz, 1H), 7.92 (dd, J = 12.8, 2.8 Hz, 1H), 7.74 (dt, J = 9.2, 2.0 Hz, 1H), 7.42 (dd, J = 8.0, 4.8 Hz, 1H), 7.24 (t, J = 9.2 Hz, 1H), 5.96 (s, 1H), 5.16 (s, 2H), 2.19 (s, 3H), 1.47 (s, 6H). | 476.2 |
| | | 4-((3-chloropyridin-2-yl)methyl)- | | | |
| | | 2-(3-fluoro-4-((2-(2-hydroxy- | | | |
| | | propan-2-yl)-4-methylthiazol-5- | | | |
| | | yl)oxy)phenyl)-2,4-dihydro-3H- | | | |
| | | 1,2,4-triazol-3-one | | | |
| Example 467 Method 79 | +++ | 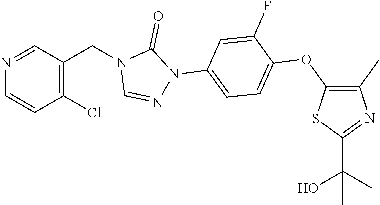 [SMILES: Cc1nc(C(C)(C)O)sc1Oc1ccc(-n2ncn(Cc3cnccc3Cl)c2=O)cc1F] | white solid, yield: 7.0% | ¹H NMR (400 MHz, DMSO-d₆) δ 8.63 (s, 1H), 8.54 (d, J = 5.2 Hz, 1H), 8.36 (s, 1H), 7.89 (dd, J = 12.8, 2.4 Hz, 1H), 7.70 (dt, J = 9.2, 2.0 Hz, 1H), 7.62 (d, J = 5.2 Hz, 1H), 7.22 (t, J = 9.2 Hz, 1H), 5.95 (brs, 1H), 5.03 (s, 2H), 2.18 (s, 3H), 1.46 (s, 6H). | 476.2 |
| | | 4-((4-chloropyridin-3-yl)methyl)- | | | |
| | | 2-(3-fluoro-4-((2-(2-hydroxy- | | | |
| | | propan-2-yl)-4-methylthiazol-5- | | | |
| | | yl)oxy)phenyl)-2,4-dihydro-3H- | | | |
| | | 1,2,4-triazol-3-one | | | |
| Example 468 Method 79 | ++++ | 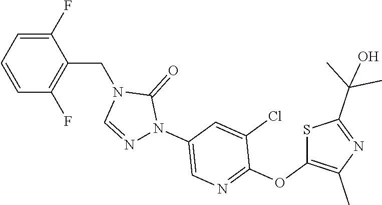 [SMILES: Cc1nc(C(C)(C)O)sc1Oc1ncc(-n2ncn(Cc3c(F)cccc3F)c2=O)cc1Cl] | light brown solid, yield: 21.6% | ¹H NMR (400 MHz, Chloroform-d) δ 8.70 (d, J = 2.4 Hz, 1H), 8.53 (d, J = 2.4 Hz, 1H), 7.61 (s, 1H), 7.38 (tt, J = 8.8, 6.4 Hz, 1H), 6.99 (dd, J = 9.2, 6.4 Hz, 2H), 4.98 (s, 2H), 2.97 (brs, 1H), 2.35 (s, 3H), 1.66 (s, 6H). | 494.2 |
| | | 2-(5-chloro-6-((2-(2-hydroxypro- | | | |
| | | pan-2-yl)-4-methylthiazol-5-yl) | | | |
| | | oxy)pyridin-3-yl)-4-(2,6-difluoro- | | | |
| | | benzyl)-2,4-dihydro-3H-1,2,4- | | | |
| | | triazol-3-one | | | |
| Example 469 Method 79: | ++++ | 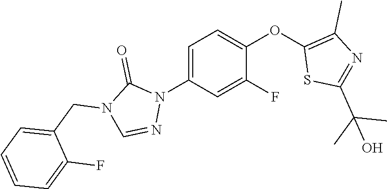 [SMILES: Cc1nc(C(C)(C)O)sc1Oc1ccc(-n2ncn(Cc3ccccc3F)c2=O)cc1F] | white solid, yield: 39.0% | ¹H NMR (400 MHz, DMSO-d₆) δ 8.35 (s, 1H), 7.90 (dd, J = 12.6, 2.5 Hz, 1H), 7.71 (ddd, J = 9.1, 2.6, 1.5 Hz, 1H), 7.43-7.40 (m, 2H), 7.31-7.18 (m, 3H), 5.94 (s, 1H), 4.96 (s, 2H), 2.19 (s, 3H), 1.47 (s, 6H). | 459.1 |
| | | 2-(3-fluoro-4-((2-(2-hydroxypro- | | | |
| | | pan-2-yl)-4-methylthiazol-5-yl) | | | |
| | | oxy)phenyl)-4-(2-fluorobenzyl)- | | | |
| | | 2,4-dihydro-3H-1,2,4-triazol-3- | | | |
| | | one | | | |
| Example 470 Method 79: | ++++ | 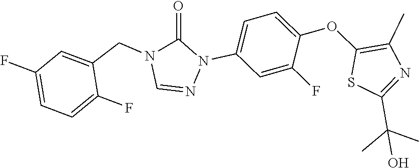 [SMILES: Cc1nc(C(C)(C)O)sc1Oc1ccc(-n2ncn(Cc3cc(F)ccc3F)c2=O)cc1F] | white solid, yield: 40.0% | ¹H NMR (400 MHz, DMSO-d₆) δ 8.36 (s, 1H), 7.89 (dd, J = 12.6, 2.5 Hz, 1H), 7.70 (dt, J = 9.2, 1.9 Hz, 1H), 7.39-7.16 (m, 4H), 5.94 (s, 1H), 4.95 (s, 2H), 2.19 (s, 3H), 1.47 (s, 6H). | 477.1 |
| | | 4-(2,5-difluorobenzyl)-2-(3-fluoro- | | | |
| | | 4-((2-(2-hydroxypropan-2-yl)- | | | |
| | | 4-methylthiazol-5-yl)oxy)phenyl)- | | | |
| | | 2,4-dihydro-3H-1,2,4-triazol- | | | |
| | | 3-one | | | |
| Example 471 | ++ | 4-((4-chloropyridin-2-yl)methyl)- | white solid, | ¹H NMR (400 MHz, DMSO-d₆) | 476.1 |
| Method 79: | | 2-(3-fluoro-4-((2-(2-hydroxy- | yield: 6.1% | δ 8.51 (d, J = 5.4 Hz, 1H), 8.37 (s, | |
| | | propan-2-yl)-4-methylthiazol-5- | | 1H), 7.90 (dd, J = 12.6, 2.5 Hz, | |
| | | yl)oxy)phenyl)-2,4-dihydro-3H- | | 1H), 7.76-7.69 (m, 1H), 7.63 (d, | |
| | | 1,2,4-triazol-3-one | | J = 2.0 Hz, 1H), 7.50 (dd, J = 5.4, | |
| | | | | 2.1 Hz, 1H), 7.23 (t, J = 9.1 Hz, | |
| | | | | 1H), 5.95 (s, 1H), 5.04 (s, 2H), | |
| | | | | 2.19 (s, 3H), 1.47 (s, 6H). | |
| Example 472 | +++ | 4-(2,6-difluorobenzyl)-2-(3-fluoro- | yellow oil, | ¹H NMR (400 MHz, CDCl₃) δ | 461.0 |
| Method 79 | | 4-((3-(2-hydroxypropan-2-yl)- | yield: | 7.97 (dd, J = 11.6, 2.0 Hz, 1H), | |
| | | 4-methylisoxazol-5-yl)oxy) | 10.3% | 7.80 (d, J = 9.2 Hz, 1H), 7.62 (s, | |
| | | phenyl)-2,4-dihydro-3H-1,2,4- | | 1H), 7.40-7.38 (m, 1H), 7.21 (t, | |
| | | triazol-3-one | | J = 8.4, 1H), 7.01 (t, J = 7.6 Hz, | |
| | | | | 2H), 5.00 (s, 2H), 2.24 (brs, 1H), | |
| | | | | 2.03 (s, 3H), 1.40 (s, 6H). | |
| Example 473 Method 79 | ++++ | 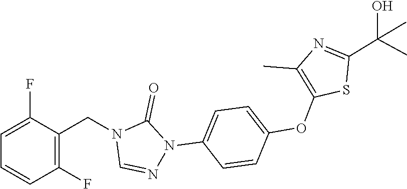 [SMILES: Cc1nc(C(C)(C)O)sc1Oc1ccc(-n2ncn(Cc3c(F)cccc3F)c2=O)cc1] | white solid, yield: 39.78% | ¹H NMR (400 MHz, DMSO-d₆) δ 8.29 (s, 1H), 7.91-7.76 (m, 2H), 7.52-7.44 (m, 1H), 7.19-7.07 (m, 4H), 5.95 (s, 1H), 4.97 (s, 2H), 2.13 (s, 3H), 1.47 (s, 6H). | 458.1 |
| | | 4-(2,6-difluorobenzyl)-2-(4-((2- | | | |
| | | (2-hydroxypropan-2-yl)-4-methyl- | | | |
| | | thiazol-5-yl)oxy)phenyl)-2,4- | | | |
| | | dihydro-3H-1,2,4-triazol-3-one | | | |

### Example 474: Method 80

### 4-(2,6-difluorobenzyl)-2-(4-((2-((4,4-dimethyl-4,5-dihydrooxazol-2-yl)amino)-5-fluoropyridin-4-yl)oxy)-3-fluorophenyl)-2,4-dihydro-3H-1,2,4- triazol-3-one

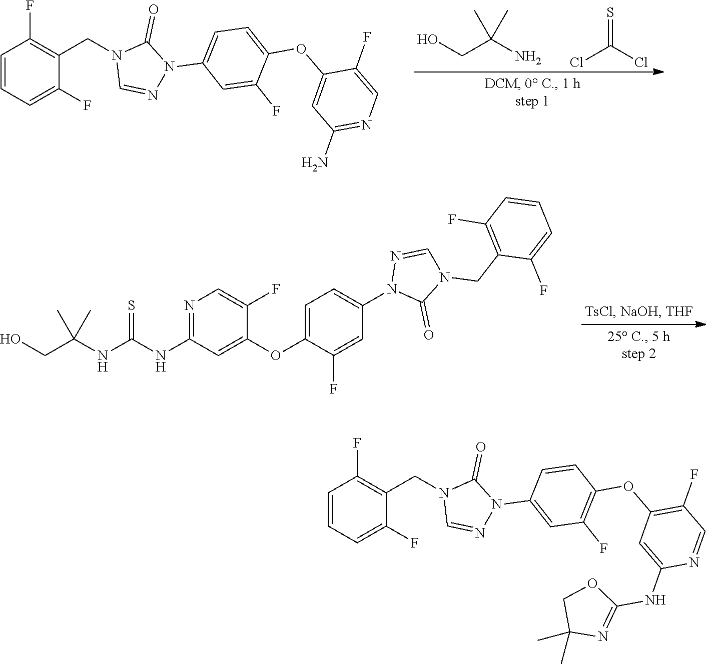

Step 1: To a solution of 2-(4-((2-amino-5-fluoropyridin-4-yl)oxy)-3-fluorophenyl)-4-(2,6-difluorobenzyl)-2,4-dihydro-3H-1,2,4-triazol-3-one (1.00 eq, 50 mg, 0.104 mmol), 2-amino-2-methyl-propan-1-ol (1.20 eq, 11.2 mg, 0.125 mmol) and Et₃N (3.00 eq, 32 mg, 0.313 mmol) in DCM (1 mL), was added thiocarbonyl dichloride (1.20 eq, 14 mg, 0.125 mmol) at 0° C. under N₂. After addition, the mixture was stirred at 0° C. for 1 h. LCMS indicated that 50% SM remained, with 30% product conversion. Then the mixture was added with water (20 mL) and extracted with DCM (20 mL×3). The organics were then combined and dried (Na₂SO₄) before concentration to dryness. The crude was purified with Prep-TLC (PE/EtOAc=1/1) to give the product (25 mg, 0.0378 mmol, 36.21% yield) as a white solid. MS (m/z): 563.1 [M+H]⁺.

Step 2: To a solution of 1-(4-(4-(4-(2,6-difluorobenzyl)-5-oxo-4,5-dihydro-1H-1,2,4-triazol-1-yl)-2-fluorophenoxy)-5-fluoropyridin-2-yl)-3-(1-hydroxy-2-methylpropan-2-yl)thiourea (1.00 eq, 25 mg, 0.0378 mmol) in THF (2 mL), were added NaOH (5.00 eq, 7.6 mg, 0.189 mmol) and TsCl (2.00 eq, 14 mg, 0.0756 mmol) at room temperature. After addition, the mixture was stirred at 25° C. for 5 h. LCMS indicated the reaction was completed. The mixture was extracted with EtOAc (20 mL×3). The organic layers were then combined and dried (Na₂SO₄) before concentration to dryness. The crude was purified with prep-TLC (100% EtOAc-0.11% NH₄₀H as additive) to give the product (4.0 mg, 0.00727 mmol, 19.24% yield) as a white solid. MS (m/z): 529.2 [M+H]⁺. ¹H NMR (400 MHz, DMSO-d₆) δ 9.00 (s, 1H), 8.38 (s, 1H), 8.24 (d, J=3.0 Hz, 1H), 7.94 (dd, J=12.4, 2.5 Hz, 1H), 7.83-7.77 (m, 1H), 7.56-7.41 (m, 2H), 7.23-7.08 (m, 2H), 6.12 (s, 1H), 5.00 (s, 2H), 3.96 (s, 2H), 1.22 (s, 6H).

| Compound No. | | | Appearance | | MS(m/z) |
|---|---|---|---|---|---|
| Method | Activity | Structure and Name | and Yield | ¹H NMR Data | [M + H]⁺ |
| Example 474 Method 80 | +++ | 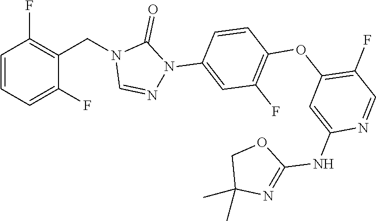 [SMILES: CC1(C)COC(Nc2cc(Oc3ccc(-n4ncn(Cc5c(F)cccc5F)c4=O)cc3F)c(F)cn2)=N1] | white solid, yield: 19.24% | ¹H NMR (400 MHz, DMSO-d₆) δ 9.00 (s, 1H), 8.38 (s, 1H), 8.24 (d, J = 3.0 Hz, 1H), 7.94 (dd, J = 12.4, 2.5 Hz, 1H), 7.83-7.77 (m, 1H), 7.56-7.41 (m, 2H), 7.23-7.08 (m, 2H), 6.12 (s, 1H), 5.00 (s, 2H), 3.96 (s, 2H), 1.22 (s, 6H). | 529.2 |
| | | 4-(2,6-difluorobenzyl)-2-(4-((2- | | | |
| | | ((4,4-dimethyl-4,5-dihydrooxazol- | | | |
| | | 2-yl)amino)-5-fluoropyridin- | | | |
| | | 4-yl)oxy)-3-fluorophenyl)-2,4- | | | |
| | | dihydro-3H-1,2,4-triazol-3-one | | | |

### Example 475: Method 81

### 5-(4-(4-(2,6-difluorobenzyl)-5-oxo-4,5-dihydro-1H-1,2,4-triazol-1-yl)-2-fluorophenoxy)-4-methylisoxazole-3-carbonitrile

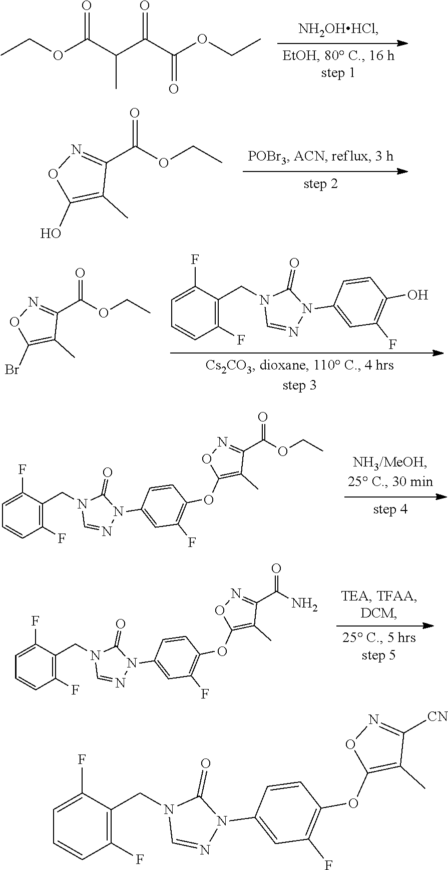

### Step 1: ethyl 5-hydroxy-4-methylisoxazole-3-carboxylate

The solution of 1,4-diethyl 2-methyl-3-oxobutanedioate (15 g, 74.2 mmol) and hydroxylamine hydrochloride (8.8 g, 126 mmol) in EtOH (150 mL) was stirred at 80° C. for 16 hrs. The mixture was cool down to r.t. The resulted mixture was diluted with water (100 mL). The resulted mixture was extracted with EtOAc (3×80 mL). The combined organic layers were washed with brine (100 mL), dried over anhydrous Na₂SO₄, and after filtration, the filtrate was concentrated under reduced pressure. The residue was purified by silica gel, eluted with (PE:EtOAc=20:1) to afford ethyl 5-hydroxy-4-methylisoxazole-3-carboxylate (8.0 g, 59.8% yield) as a white solid. MS (m/z): 172.0 [M+H]⁺.

### Step 2: ethyl 5-bromo-4-methylisoxazole-3-carboxylate

To a stirred solution of ethyl 3-hydroxy-4-methyl-1,2-oxazole-5-carboxylate (8.0 g, 46.7 mmol) in MeCN (160 mL) was added POBr₃ (92.1 mg, 0.32 mmol). The reaction mixture was heated to reflux for 3 hours. The mixture was allowed to cool down to rt. The resulted mixture was diluted with sodium bicarbonate solution (100 mL). The resulted mixture was extracted with EtOAc (3×50 mL). The combined organic layers were washed with brine (50 mL), dried over anhydrous Na₂SO₄, and after filtration, the filtrate was concentrated under reduced pressure. The residue was purified by silica gel, eluted with (PE:EtOAc=40:1) to afford ethyl 5-bromo-4-methylisoxazole-3-carboxylate (9.0 g, 78.1% yield) as a yellow oil. LC-MS (m/z): 235.0[M+H]⁺.

### Step 3: ethyl 5-(4-(4-(2,6-difluorobenzyl)-5-oxo-4,5-dihydro-1H-1,2,4-triazol-1-yl)-2-fluorophenoxy)-4-methylisoxazole-3-carboxylate

To a stirred solution of 4-(2,6-difluorobenzyl)-2-(3-fluoro-4-hydroxyphenyl)-2,4-dihydro-3H-1,2,4-triazol-3-one (300 mg, 0.93 mmol) in 1,4-dioxane (6 mL) were added ethyl 5-bromo-4-methylisoxazole-3-carboxylate (327 mg, 1.4 mmol) and Cs₂CO₃ (912 mg, 2.8 mmol) under nitrogen. The mixture was stirred at 110° C. for 4 hours. The mixture was allowed to cool down to r.t. The resulted mixture was diluted with water (10 mL). The resulted mixture was extracted with EtOAc (3×5 mL). The combined organic layers were washed with brine (10 mL), dried over anhydrous Na₂SO₄, and after filtration, the filtrate was concentrated under reduced pressure. The residue was purified by Prep-TLC (PE/EA=2:1) to afford the titled product (110 mg, 23.6% yield) as a yellow solid. LC-MS (m/z): 475.0[M+H]⁺. ¹H NMR (400 MHz, CDCl₃) δ 7.98 (dd, J=12.0, 2.4 Hz, 1H), 7.84-7.81 (m, 1H), 7.62 (s, 1H), 7.44-7.35 (m, 1H), 7.21 (t, J=8.8 Hz, 1H), 7.06-6.96 (m, 2H), 5.00 (s, 2H), 4.48-4.43 (m, 2H), 2.09 (s, 3H), 1.44 (t, J=6.8 Hz, 3H).

### Step 4: 5-(4-(4-(2,6-difluorobenzyl)-5-oxo-4,5-dihydro-1H-1,2,4-triazol-1-yl)-2-fluorophenoxy)-4-methylisoxazole-3-carboxamide

To a stirred solution of ethyl 5-(4-(4-(2,6-difluorobenzyl)-5-oxo-4,5-dihydro-1H-1,2,4-triazol-1-yl)-2-fluorophenoxy)-4-methylisoxazole-3-carboxylate (50 mg, 0.10 mmol) in MeOH (2 mL) was added MeOH/NH₃ (2 mL) at 25° C. The mixture was stirred at 25° C. for 30 min. The mixture was concentrated under reduced pressure. The residue was purified by Prep-TLC (PE/EA=2:1) to afford the titled product (15 mg, 30.36% yield) as an off-white solid. LC-MS (m/z): 446.0 [M+H]⁺. ¹H NMR (400 MHz, CDCl₃) δ 7.98 (dd, J=12.0, 2.4 Hz, 1H), 7.85-7.82 (m, 1H), 7.62 (s, 1H), 7.41-7.37 (m, 1H), 7.28-7.22 (m, 1H), 7.03-6.99 (m, 2H), 4.99 (s, 2H), 2.12 (s, 3H).

### Step 5: 5-(4-(4-(2,6-difluorobenzyl)-5-oxo-4,5-dihydro-1H-1,2,4-triazol-1-yl)-2-fluorophenoxy)-4-methylisoxazole-3-carbonitrile

To a solution of 5-(4-(4-(2,6-difluorobenzyl)-5-oxo-4,5-dihydro-1H-1,2,4-triazol-1-yl)-2-fluorophenoxy)-4-methylisoxazole-3-carboxamide (20 mg, 0.04 mmol) in DCM (5 mL) were added triethylamine (23 mg, 0.22 mmol) and TFAA (28 mg, 0.13 mmol) under nitrogen at 0° C. The mixture was stirred at 25° C. for 5h, and then the reaction was quenched by ice-water. The mixture was extracted with DCM (50 mL×3). The combined organic layers were washed with water (50 mL×1), brine (50 mL), dried over Na₂SO₄ and concentrated. The residue was purified by prep-TLC (PE/EA=10:1) to give the titled product (5 mg, 24.7% yield) as a white solid. LC-MS (m/z): 428.0 [M+H]⁺. ¹H NMR (400 MHz, CDCl₃) δ 8.01 (dd, J=11.6, 2.0 Hz, 1H), 7.89 (d, J=9.2 Hz, 1H), 7.63 (s, 1H), 7.42-7.38 (m, 1H), 7.31 (s, 1H), 7.01 (t, J=8.0 Hz, 1H), 5.00 (s, 2H), 2.02 (s, 3H).

The synthesis procedure of Examples (Compounds) 476-477 were similar to the procedure of Example 475.

| Compound | | | | | |
|---|---|---|---|---|---|
| No. | | | Appearance | | MS(m/z) |
| Method | Activity | Structure and Name | and Yield | ¹H NMR Data | [M + H]⁺ |
| Example 475 Method 81 | ++++ | 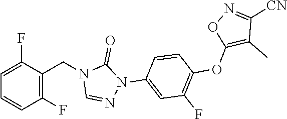 [SMILES: Cc1c(C#N)noc1Oc1ccc(-n2ncn(Cc3c(F)cccc3F)c2=O)cc1F] 5-(4-(4-(2,6-difluorobenzyl)-5- oxo-4,5-dihydro-1H-1,2,4-triazol- 1-yl)-2-fluorophenoxy)-4-methyl- isoxazole-3-carbonitrile | white solid, yield: 24.7% | ¹H NMR (400 MHZ, CDCl₃) δ 8.01 (dd, J = 11.6, 2.0 Hz, 1H), 7.89 (d, J = 9.2 Hz, 1H), 7.63 (s, 1H), 7.42-7.38 (m, 1H), 7.31 (s, 1H), 7.01 (t, J = 8.0 Hz, 1H), 5.00 (s, 2H), 2.02 (s, 3H). | 428.0 |
| Example 476 Method 81 | ++++ | 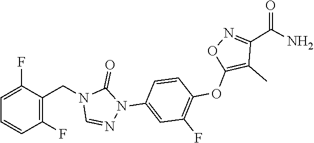 [SMILES: Cc1c(C(N)=O)noc1Oc1ccc(-n2ncn(Cc3c(F)cccc3F)c2=O)cc1F] 5-(4-(4-(2,6-difluorobenzyl)-5- oxo-4,5-dihydro-1H-1,2,4-triazol- 1-yl)-2-fluorophenoxy)-4-methyl- isoxazole-3-carboxamide | off-white solid, yield: 30.36% | ¹H NMR (400 MHZ, CDCl₃) δ 7.98 (dd, J = 12.0, 2.4 Hz, 1H), 7.85-7.82 (m, 1H), 7.62 (s, 1H), 7.41-7.37 (m, 1H), 7.28-7.22 (m, 1H), 7.03-6.99 (m, 2H), 4.99 (s, 2H), 2.12 (s, 3H). | 446.0 |
| Example 477 Method 81 | +++ | 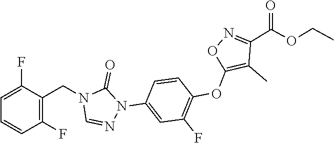 [SMILES: CCOC(=O)c1noc(Oc2ccc(-n3ncn(Cc4c(F)cccc4F)c3=O)cc2F)c1C] ethyl 5-(4-(4-(2,6-difluorobenzyl)-5- oxo-4,5-dihydro-1H-1,2,4-triazol- 1-yl)-2-fluorophenoxy)-4-methyl- isoxazole-3-carboxylate | yellow solid, yield: 23.6% | ¹H NMR (400 MHZ, CDCl₃) δ 7.98 (dd, J = 12.0, 2.4 Hz, 1H), 7.84-7.81 (m, 1H), 7.62 (s, 1H), 7.44-7.35 (m, 1H), 7.21 (t, J = 8.8 Hz, 1H), 7.06-6.96 (m, 2H), 5.00 (s, 2H), 4.48-4.43 (m, 2H), 2.09 (s, 3H), 1.44 (t, J = 6.8 Hz, 3H). | 475.0 |

### Example 478: Method 82

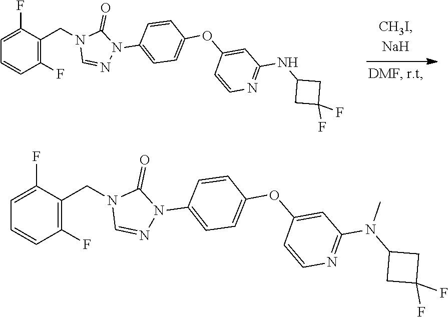 [SMILES: O=c1n(Cc2c(F)cccc2F)cnn1-c1ccc(Oc2ccnc(NC3CC(F)(F)C3)c2)cc1 ; CN(c1cc(Oc2ccc(-n3ncn(Cc4c(F)cccc4F)c3=O)cc2)ccn1)C1CC(F)(F)C1]

To a solution of 4-[(2,6-difluorophenyl)methyl]-2-(4-hydroxyphenyl)-1,2,4-triazol-3-one (1.00 eq, 50 mg, 0.103 mmol) in DMF (4 mL), were added NaH (1.20 eq, 5 mg, 0.120 mmol), CH₃I (2 eq, 29 mg, 0.206 mmol). The reaction mixture was stirred for 0.5 h at 25° C. under nitrogen. The reaction was concentrated to dryness and the residue was taken up in EA (20 mL). The organics were washed with 2×20 mL water and then 1×20 mL saturated brine solution. The organics were then separated and dried (MgSO₄) before concentration to dryness. The crude was then purified by flash column chromatography eluted with 80% ACN in H₂O. The desired fractions were concentrated to dryness in vacuo to afford 4-(2,6-difluorobenzyl)-2-(4-((2-((3,3-difluorocyclobutyl)(methyl)amino)pyridin-4-yl)oxy)phen yl)-2,4-dihydro-3H-1,2,4-triazol-3-one (7 mg, 13.3% yield) as a white solid. MS (m/z): 500.2 [M+H]⁺. ¹H NMR (400 MHz, Chloroform-d) δ 8.25-8.08 (m, 3H), 7.63 (s, 1H), 7.44-7.32 (m, 1H), 7.15 (d, J=8.8 Hz, 2H), 7.00 (dd, J=8.4, 7.2 Hz, 2H), 6.53 (d, J=6.0 Hz, 1H), 6.05 (s, 1H), 5.00 (s, 2H), 4.53-4.34 (m, 1H), 3.17 (s, 3H), 3.12-3.00 (m, 2H), 2.82-2.68 (m, 2H).

The synthesis procedure of Example (Compound) 479 was similar to the procedure of Example 478.

| Compound | | | | | |
|---|---|---|---|---|---|
| No. | | | Appearance | | MS(m/z) |
| Method | Activity | Structure and Name | and Yield | ¹H NMR Data | [M + H]⁺ |
| Example 478 Method 82 | +++ | 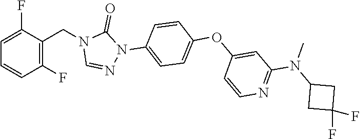 [SMILES: CN(c1cc(Oc2ccc(-n3ncn(Cc4c(F)cccc4F)c3=O)cc2)ccn1)C1CC(F)(F)C1] 4-(2,6-difluorobenzyl)-2-(4-((2- ((3,3-difluorocyclobutyl)(methy 1)amino)pyridin-4-yl)oxy)pheny 1)-2,4-dihydro-3H-1,2,4-triazol- 3-one | white solid, yield: 13.3 % | ¹H NMR (400 MHZ, Chloroform-d) δ 8.25-8.08 (m, 3H), 7.63 (s, 1H), 7.44-7.32 (m, 1H), 7.15 (d, J = 8.8 Hz, 2H), 7.00 (dd, J = 8.4, 7.2 Hz, 2H), 6.53 (d, J = 6.0 Hz, 1H), 6.05 (s, 1H), 5.00 (s, 2H), 4.53-4.34 (m, 1H), 3.17 (s, 3H), 3.12-3.00 (m, 2H), 2.82-2.68 (m, 2H). | 500.2 |
| Example 479 Method 82 | ++ | 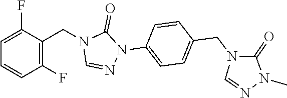 [SMILES: Cn1ncn(Cc2ccc(-n3ncn(Cc4c(F)cccc4F)c3=O)cc2)c1=O] 4-(2,6-difluorobenzyl)-2-(4-((1- methyl-5-oxo-1,5-dihydro-4H-1, 2,4-triazol-4-yl)methyl)phenyl)- 2,4-dihydro-3H-1,2,4-triazol-3- one | white solid, yield: 16.2 % | ¹H NMR (400 MHZ, Chloroform-d) δ 8.03-7.95 (m, 2H), 7.58 (s, 1H), 7.42-7.31 (m, 3H), 7.25 (s, 1H), 7.03-6.93 (m, 2H), 4.97 (d, J = 1.2 Hz, 2H), 4.78 (s, 2H), 3.48 (s, 3H). | 399.2 |

### Example 480: Method 83

### tert-butyl2-(4-(4-(4-(2,6-difluorobenzyl)-5-oxo-4,5-dihydro-1H-1,2,4-triazol-1-yl)phenoxy)pyridin-2-yl)-2,7-diazaspiro[3.5]nonane-7-carboxylate

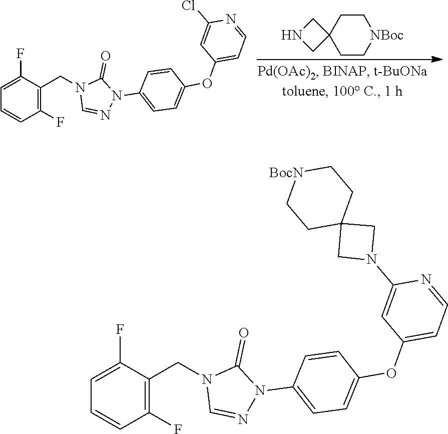 [SMILES: O=c1n(Cc2c(F)cccc2F)cnn1-c1ccc(Oc2ccnc(Cl)c2)cc1 ; CC(C)(C)OC(=O)N1CCC2(CC1)CNC2 ; CC(C)(C)OC(=O)N1CCC2(CC1)CN(c1cc(Oc3ccc(-n4ncn(Cc5c(F)cccc5F)c4=O)cc3)ccn1)C2]

To a solution of 2-(4-((2-chloropyridin-4-yl)oxy)phenyl)-4-(2,6-difluorobenzyl)-2,4-dihydro-3H-1,2,4-triazol-3-one (100 mg, 241.1 umol) in toluene (4 mL) were added tert-butyl 2,7-diazaspiro[3.5]nonane-7-carboxylate (109 mg, 482.2 umol), Pd(OAc)₂ (54 mg, 241.1 umol), BINAP (150 mg, 241.1 umol) and t-BuONa (93 mg, 964.3 umol) under Ar. The reaction mixture was stirred at 100° C. for 1 h. The target product was detected by LCMS. The reaction mixture was taken up in EtOAc (100 mL) and the organics washed with 2×50 mL water and then 1×50 mL saturated brine solution. The organics were then separated and dried (MgSO₄) before concentration to dryness. The crude was then purified by reversed-phase column to obtain tert-butyl2-(4-(4-(4-(2,6-difluorobenzyl)-5-oxo-4,5-dihydro-1H-1,2,4-triazol-1-yl)phenoxy)pyridin-2-yl)-2,7-diazaspiro[3.5]nonane-7-carboxylate (22 mg, 15.1% yield) as a white solid. MS (m/z): 605.2 [M+H]⁺. ¹H NMR (400 MHz, DMSO) δ 8.36 (s, 1H), 7.99 (t, J=7.6 Hz, 3H), 7.66-7.51 (m, 3H), 7.36-7.27 (m, 2H), 7.17 (t, J=8.1 Hz, 2H), 5.00 (s, 2H), 3.92-3.66 (m, 8H), 1.72-1.62 (m, 4H), 1.38 (s, 9H).

The synthesis procedure of Examples (Compounds) 481-506 were similar to the procedure of Example 480.

| Com- | | | Appear- | | |
|---|---|---|---|---|---|
| pound | | | ance | | MS |
| No. | | | and | | (m/z) |
| Method | Activity | Structure and Name | Yield | ¹H NMR Data | [M + H]⁺ |
| Example 480 Method 83 | ++++ | 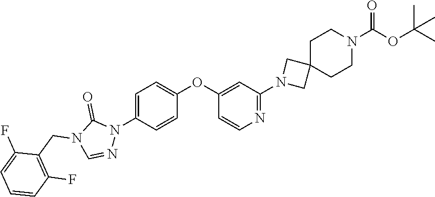 [SMILES: CC(C)(C)OC(=O)N1CCC2(CC1)CN(c1cc(Oc3ccc(-n4ncn(Cc5c(F)cccc5F)c4=O)cc3)ccn1)C2] tert-butyl2- (4-(4-(4-(2,6-difluorobenzyl)- 5-oxo-4,5-dihydro-1H-1,2,4- triazol-1-yl)phenoxy)pyridin- 2-yl)-2,7-diazaspiro[3.5]nonane- 7-carboxylate | white solid., yield: 15.1% | ¹H NMR (400 MHZ, DMSO-d₆) δ 8.36 (s, 1H), 7.99 (t, J = 7.6 Hz, 3H), 7.66-7.51 (m, 3H), 7.36-7.27 (m, 2H), 7.17 (t, J = 8.1 Hz, 2H), 5.00 (s, 2H), 3.92-3.66 (m, 8H), 1.72-1.62 (m, 4H), 1.38 (s, 9H). | 605.2 |
| Example 481 Method 83 | +++ | 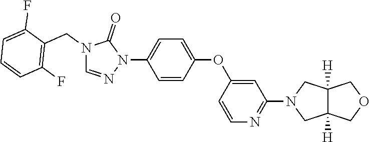 [SMILES: O=c1n(Cc2c(F)cccc2F)cnn1-c1ccc(Oc2ccnc(N3C[C@H]4COC[C@H]4C3)c2)cc1] 4-(2,6-difluorobenzyl)-2-(4-((2- ((3aR,6aS)-tetrahydro-1H-furo [3,4-c]pyrrol-5(3H)-yl)pyridin-4- yl)oxy)phenyl)-2,4-dihydro-3H- 1,2,4-triazol-3-one | white solid, yield: 13.5% | ¹H NMR (400 MHZ, DMSO-d₆) δ 8.36 (s, 1H), 8.09-7.93 (m, 3H), 7.49 (tt, J = 8.5, 6.6 Hz, 1H), 7.40-7.30 (m, 2H), 7.17 (t, J = 8.1 Hz, 2H), 6.55 (dd, J = 7.0, 2.3 Hz, 1H), 6.22 (d, J = 2.4 Hz, 1H), 5.00 (s, 2H), 3.78 (dd, J = 8.9, 6.4 Hz, 2H), 3.67-3.59 (m, 2H), 3.37 (dd, J = 11.1, 3.2 Hz, 2H), 3.08 (dt, J = 7.0, 3.6 Hz, 2H), 1.32-1.18 (m, 2H). | 492.2 |
| Example 482 Method 83: | +++ | 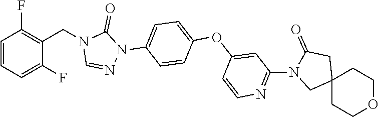 [SMILES: O=C1CC2(CCOCC2)CN1c1cc(Oc2ccc(-n3ncn(Cc4c(F)cccc4F)c3=O)cc2)ccn1] 2-(4-(4-(4-(2,6-difluorobenzyl)- 5-oxo-4,5-dihydro-1H-1,2,4- triazol-1-yl)phenoxy)pyridin-2- yl)-8-oxa-2-azaspiro[4.5]decan- 3-one | white solid, yield: 6.2% | ¹H NMR (400 MHZ, DMSO-d₆) δ 8.34 (s, 1H), 8.26 (d, J = 5.8 Hz, 1H), 8.00-7.89 (m, 2H), 7.86 (d, J = 2.3 Hz, 1H), 7.49 (tt, J = 8.5, 6.6 Hz, 1H), 7.33-7.24 (m, 2H), 7.17 (t, J = 8.1 Hz, 2H), 6.77 (dd, J = 5.8, 2.4 Hz, 1H), 4.99 (s, 2H), 3.84 (s, 2H), 3.68-3.45 (m, 4H), 2.54 (s, 2H), 1.66-1.50 (m, 4H). | 434.2 |
| Example 483 Method 83: | +++ | 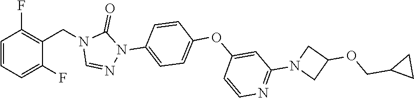 [SMILES: O=c1n(Cc2c(F)cccc2F)cnn1-c1ccc(Oc2ccnc(N3CC(OCC4CC4)C3)c2)cc1] 2-(4-((2-(3-(cyclopropylmethoxy) azetidin-1-yl)pyridin-4-yl)oxy) phenyl)-4-(2,6-difluorobenzyl)- 2,4-dihydro-3H-1,2,4-triazol-3- one | white solid, yield: 2.5% | ¹H NMR (400 MHZ, DMSO-d₆) δ 8.35 (s, 1H), 8.06-7.92 (m, 3H), 7.49 (tt, J = 8.5, 6.6 Hz, 1H), 7.35-7.28 (m, 2H), 7.23-7.12 (m, 2H), 6.47 (dd, J = 6.9, 2.3 Hz, 1H), 6.04 (d, J = 2.4 Hz, 1H), 4.99 (s, 2H), 4.58 (td, J = 6.8, 3.5 Hz, 1H), 4.34-4.21 (m, 2H), 3.84 (dd, J = 9.5, 4.3 Hz, 2H), 3.50-3.42 (m, 2H), 1.99 (p, J = 7.0, 6.5 Hz, 1H), 1.30-1.20 (m, 4H). | 506.1 |
| Example 484 Method 83: | +++ | 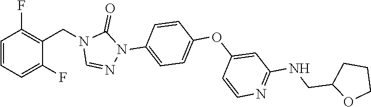 [SMILES: O=c1n(Cc2c(F)cccc2F)cnn1-c1ccc(Oc2ccnc(NCC3CCCO3)c2)cc1] 4-(2,6-difluorobenzyl)-2-(4-((2- (((tetrahydrofuran-2-yl)methyl) amino)pyridin-4-yl)oxy)phenyl)- 2,4-dihydro-3H-1,2,4-triazol-3- one | white solid, yield: 11.3% | ¹H NMR (400 MHZ, DMSO-d₆) δ 8.54 (s, 1H), 8.36 (s, 1H), 8.07-7.98 (m, 2H), 7.94 (d, J = 7.2 Hz, 1H), 7.49 (tt, J = 8.5, 6.6 Hz, 1H), 7.42-7.32 (m, 2H), 7.17 (t, J = 8.1 Hz, 2H), 6.64 (dd, J = 7.4, 2.4 Hz, 1H), 6.28 (s, 1H), 5.01 (s, 2H), 3.98-3.88 (m, 1H), 3.78-3.68 (m, 1H), 3.63 (td, J = 7.7, 6.3 Hz, 1H), 3.42 (dt, J = 14.0, 4.3 Hz, 1H), 3.35-3.18 (m, 1H), 2.02-1.88 (m, 1H), 1.91-1.73 (m, 2H), 1.53-1.50 (m, 1H). | 480.1 |
| Example 485 Method 83: | +++ | 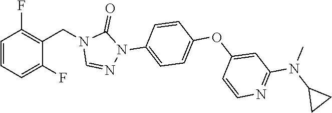 [SMILES: CN(c1cc(Oc2ccc(-n3ncn(Cc4c(F)cccc4F)c3=O)cc2)ccn1)C1CC1] 2-(4-((2-(cyclopropyl(methyl) amino)pyridin-4-yl)oxy)phenyl)- 4-(2,6-difluorobenzyl)-2,4- dihydro-3H-1,2,4-triazol-3-one | white solid, yield: 2.8% | NA | 450.1 |
| Example 486 Method 83: | +++ | 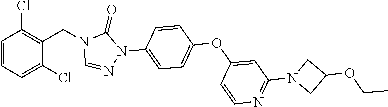 [SMILES: CCOC1CN(c2cc(Oc3ccc(-n4ncn(Cc5c(Cl)cccc5Cl)c4=O)cc3)ccn2)C1] 4-(2,6-dichlorobenzyl)-2-(4-((2- (3-ethoxyazetidin-1-yl)pyridin-4- yl)oxy)phenyl)-2,4-dihydro-3H- 1,2,4-triazol-3-one | white solid, yield: 16.6% | ¹H NMR (400 MHZ, DMSO-d₆) δ 8.17 (s, 1H), 8.06-7.93 (m, 3H), 7.58 (d, J = 8.0 Hz, 2H), 7.47 (dd, J = 8.8, 7.3 Hz, 1H), 7.36-7.27 (m, 2H), 6.47 (dd, J = 6.8, 2.4 Hz, 1H), 6.00 (d, J = 2.4 Hz, 1H), 5.12 (s, 2H), 4.39 (dq, J = 6.7, 4.0, 3.3 Hz, 1H), 4.27 (dd, J = 9.6, 6.4 Hz, 2H), 3.89 (dd, J = 9.8, 3.9 Hz, 2H), 3.50-3.42 (m, 2H), 1.13 (t, J = 7.0 Hz, 3H). | 512.1 |
| Example 487 Method 83: | +++ | 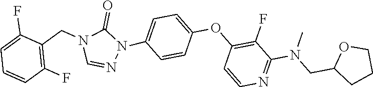 [SMILES: CN(CC1CCCO1)c1nccc(Oc2ccc(-n3ncn(Cc4c(F)cccc4F)c3=O)cc2)c1F] 4-(2,6-difluorobenzyl)-2-(4-((3- fluoro-2-(methyl((tetrahydrofuran- 2-yl)methyl)amino)pyridin-4- yl)oxy)phenyl)-2,4-dihydro-3H- 1,2,4-triazol-3-one | light- brown solid, yield: 5.9% | ¹H NMR (400 MHZ, DMSO-d₆) δ 8.31 (s, 1H), 7.94-7.84 (m, 2H), 7.79 (t, J = 6.0 Hz, 1H), 7.56-7.42 (m, 2H), 7.32-7.20 (m, 2H), 6.98-6.88 (m, 1H), 6.26 (t, J = 5.3 Hz, 1H), 4.97 (s, 2H), 4.06 (dt, J = 7.4, 3.6 Hz, 1H), 3.82-3.70 (m, 1H), 3.68-3.57 (m, 3H), 3.12 (s, 3H), 2.11-1.58 (m, 4H). | 512.1 |
| Example 488 Method 83: | ++++ | 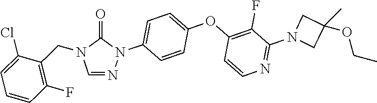 [SMILES: CCOC1(C)CN(c2nccc(Oc3ccc(-n4ncn(Cc5c(F)cccc5Cl)c4=O)cc3)c2F)C1] 4-(2-chloro-6-fluorobenzyl)-2- (4-((2-(3-ethoxy-3-methylazetidin- 1-yl)-3-fluoropyridin-4-yl)oxy) phenyl)-2,4-dihydro-3H-1,2,4- triazol-3-one | light- brown solid, yield: 9.3% | ¹H NMR (400 MHZ, DMSO-d₆) δ 8.27 (s, 1H), 7.94-7.88 (m, 2H), 7.78 (d, J = 5.6 Hz, 1H), 7.54-7.19 (m, 5H), 6.28 (t, J = 5.5 Hz, 1H), 5.03 (s, 2H), 4.41 (p, J = 5.2 Hz, 1H), 4.28 (ddd, J = 8.8, 6.2, 2.1 Hz, 2H), 3.89 (dd, J = 8.9, 4.3 Hz, 2H), 2.02-1.92 (m, 1H), 1.24 (s, 3H), 1.14 (t, J = 7.0 Hz, 3H). | 528.1 |
| Example 489 Method 83: | +++ | 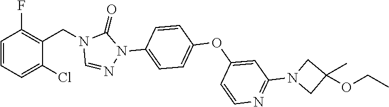 [SMILES: CCOC1(C)CN(c2cc(Oc3ccc(-n4ncn(Cc5c(F)cccc5Cl)c4=O)cc3)ccn2)C1] 4-(2-chloro-6-fluorobenzyl)-2- (4-((2-(3-ethoxy-3-methylazetidin- 1-yl)pyridin-4-yl)oxy)phenyl)- 2,4-dihydro-3H-1,2,4-triazol-3- one | off- white solid, yield: 15.% | ¹H NMR (400 MHZ, DMSO-d₆) δ 8.36 (s, 1H), 8.05-7.95 (m, 3H), 7.49 (tt, J = 8.4, 6.7 Hz, 1H), 7.39-7.28 (m, 2H), 7.23-7.11 (m, 2H), 6.56 (dd, J = 7.0, 2.4 Hz, 1H), 6.02 (d, J = 2.4 Hz, 1H), 5.00 (s, 2H), 4.06 (d, J = 9.4 Hz, 2H), 3.95 (d, J = 9.4 Hz, 2H), 3.41 (q, J = 6.9 Hz, 2H), 1.46 (s, 3H), 1.12 (t, J = 6.9 Hz, 3H). | 494.1 |
| Example 490 Method 83: | ++++ | 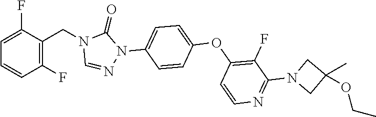 [SMILES: CCOC1(C)CN(c2nccc(Oc3ccc(-n4ncn(Cc5c(F)cccc5F)c4=O)cc3)c2F)C1] 4-(2,6-difluorobenzyl)-2-(4-((2- (3-ethoxy-3-methylazetidin-1- yl)-3-fluoropyridin-4-yl)oxy) phenyl)-2,4-dihydro-3H-1,2,4- triazol-3-one | off- white solid, yield: 13.0% | ¹H NMR (400 MHZ, DMSO-d₆) δ 8.32 (s, 1H), 7.95-7.86 (m, 2H), 7.79 (d, J = 5.7 Hz, 1H), 7.49 (tt, J = 8.5, 6.6 Hz, 1H), 7.31-7.21 (m, 2H), 7.21-7.11 (m, 2H), 6.29 (t, J = 5.6 Hz, 1H), 4.99 (s, 2H), 4.04-3.80 (m, 4H), 3.43 (q, J = 6.9 Hz, 2H), 1.49 (s, 3H), 1.13 (t, J = 6.9 Hz, 3H). | 512.1 |
| Example 491 Method 83: | ++++ | 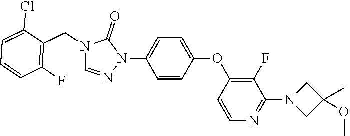 [SMILES: COC1(C)CN(c2nccc(Oc3ccc(-n4ncn(Cc5c(F)cccc5Cl)c4=O)cc3)c2F)C1] 4-(2-chloro-6-fluorobenzyl)-2- (4-((3-fluoro-2-(3-methoxy-3- methylazetidin-1-yl)pyridin-4-yl) oxy)phenyl)-2,4-dihydro-3H-1,2, 4-triazol-3-one | white solid, yield: 9.2% | ¹H NMR (400 MHZ, DMSO-d₆) δ 8.32 (s, 1H), 7.95-7.86 (m, 2H), 7.79 (d, J = 5.7 Hz, 1H), 7.49 (tt, J = 8.4, 6.6 Hz, 1H), 7.28-7.21 (m, 2H), 7.21-7.11 (m, 2H), 6.29 (t, J = 5.5 Hz, 1H), 4.98 (s, 2H), 4.03 (d, J = 8.7 Hz, 2H), 3.90 (dd, J = 9.3, 2.2 Hz, 2H), 3.20 (s, 3H), 1.48 (s, 3H). | 498.1 |
| Example 492 Method 83: | ++++ | 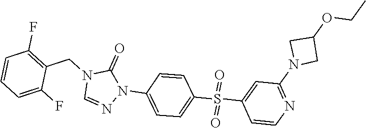 [SMILES: CCOC1CN(c2cc(S(=O)(=O)c3ccc(-n4ncn(Cc5c(F)cccc5F)c4=O)cc3)ccn2)C1] 4-(2,6-difluorobenzyl)-2-(4-((2- (3-ethoxyazetidin-1-yl)pyridin-4- yl)sulfonyl)phenyl)-2,4-dihydro- 3H-1,2,4-triazol-3-one | White solid, yield: 7.1% | ¹H NMR (400 MHZ, DMSO-d₆) δ 8.42 (s, 1H), 8.24 (d, J = 5.2 Hz, 1H), 8.20-8.12 (m, 2H), 8.12-8.04 (m, 2H), 7.48 (tt, J = 8.5, 6.7 Hz, 1H), 7.16 (t, J = 8.1 Hz, 2H), 6.96 (dd, J = 5.3, 1.6 Hz, 1H), 6.79 (d, J = 1.6 Hz, 1H), 4.98 (s, 2H), 4.22 (dd, J = 9.3, 6.3 Hz, 2H), 3.82 (dd, J = 9.4, 4.0 Hz, 2H), 3.44 (q, J = 7.0 Hz, 2H), 1.14 (t, J = 7.0 Hz, 3H). | 528.1 |
| Example 493 Method 83: | ++++ | 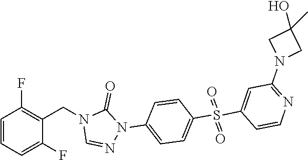 [SMILES: CC1(O)CN(c2cc(S(=O)(=O)c3ccc(-n4ncn(Cc5c(F)cccc5F)c4=O)cc3)ccn2)C1] 4-(2,6-difluorobenzyl)-2-(4-((2- (3-hydroxy-3-methylazetidin-1- yl)pyridin-4-yl)sulfonyl)phenyl)- 2,4-dihydro-3H-1,2,4-triazol-3- one | White solid, yield: 8.2% | ¹H NMR (400 MHZ, DMSO-d₆) δ 8.42 (s, 1H), 8.24 (d, J = 5.3 Hz, 1H), 8.19-8.11 (m, 2H), 8.11-8.03 (m, 2H), 7.48 (tt, J = 8.4, 6.6 Hz, 1H), 7.16 (t, J = 8.1 Hz, 2H), 6.95 (dd, J = 5.3, 1.6 Hz, 1H), 6.78 (d, J = 1.6 Hz, 1H), 4.98 (s, 2H), 3.90 (d, J = 8.6 Hz, 2H), 3.83 (d, J = 8.5 Hz, 2H), 1.42 (s, 3H). | 514.1 |
| Example 494 Method 83: | ++++ | 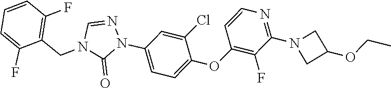 [SMILES: CCOC1CN(c2nccc(Oc3ccc(-n4ncn(Cc5c(F)cccc5F)c4=O)cc3Cl)c2F)C1] 2-(3- chloro-4-((2-(3-ethoxyazetidin- 1-yl)-3-fluoropyridin-4-yl)oxy) phenyl)-4-(2,6-difluorobenzyl)- 2,4-dihydro-3H-1,2,4-triazol- 3-one | White solid, yield: 21.2% | ¹H NMR (400 MHZ, DMSO-d₆) δ 8.40 (s, 1H), 8.15 (dd, J = 8.7, 2.9 Hz, 2H), 7.91 (dd, J = 9.0, 2.5 Hz, 1H), 7.55-7.43 (m, 2H), 7.22-7.11 (m, 2H), 5.70 (d, J = 5.7 Hz, 1H), 5.00 (s, 2H), 4.30 (tt, J = 6.3, 4.2 Hz, 1H), 4.04 (dd, J = 9.1, 6.3 Hz, 2H), 3.64 (dd, J = 9.1, 4.2 Hz, 2H), 3.38 (q, J = 7.0 Hz, 2H), 1.09 (t, J = 7.0 Hz, 3H). | 532.1 |
| Example 495 Method 83: | ++++ | 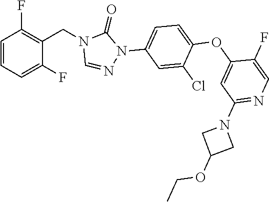 [SMILES: CCOC1CN(c2cc(Oc3ccc(-n4ncn(Cc5c(F)cccc5F)c4=O)cc3Cl)c(F)cn2)C1] 2-(3-chloro-4-((2-(3- ethoxyazetidin-1-yl)-5- fluoropyridin-4-yl)oxy)phenyl)- 4-(2,6-difluorobenzyl)-2,4- dihydro-3H-1,2,4-triazol-3-one | White solid, yield: 14.7% | ¹H NMR (400 MHZ, DMSO-d₆) δ 8.39 (s, 1H), 8.12 (d, J = 2.6 Hz, 1H), 7.89 (dd, J = 9.0, 2.6 Hz, 1H), 7.76 (d, J = 5.7 Hz, 1H), 7.57-7.37 (m, 2H), 7.17 (t, J = 8.1 Hz, 2H), 6.16 (t, J = 5.5 Hz, 1H), 4.99 (s, 2H), 4.03 (d, J = 8.7 Hz, 2 H), 3.90 (dd, J = 9.0, 2.2 Hz, 2H), 3.20 (s, 3H), 1.48 (s, 3H). | 532.1 |
| Example 496 Method 83: | ++++ | 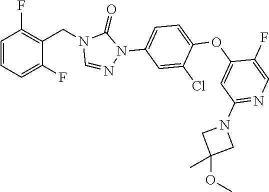 [SMILES: COC1(C)CN(c2cc(Oc3ccc(-n4ncn(Cc5c(F)cccc5F)c4=O)cc3Cl)c(F)cn2)C1] 2-(3-chloro-4-((5-fluoro-2-(3- methoxy-3-methylazetidin-1-yl) pyridin-4-yl)oxy)phenyl)-4-(2,6- difluorobenzyl)-2,4-dihydro-3H- 1,2,4-triazol-3-one | White solid, yield: 14.6% | ¹H NMR (400 MHz, DMSO-d₆) δ 8.39 (s, 1H), 8.12 (d, J = 2.6 Hz, 1H), 7.89 (dd, J = 9.0, 2.6 Hz, 1H), 7.76 (d, J = 5.7 Hz, 1H), 7.57-7.36 (m, 2H), 7.17 (t, J = 8.1 Hz, 2H), 6.16 (t, J = 5.5 Hz, 1H), 4.99 (s, 2H), 4.03 (d, J = 8.7 Hz, 2H), 3.90 (dd, J = 9.0, 2.2 Hz, 2H), 3.20 (s, 3H), 1.48 (s, 3H). | 532.1 |
| Example 497 Method 83: | ++++ | 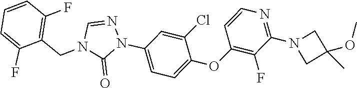 [SMILES: COC1(C)CN(c2nccc(Oc3ccc(-n4ncn(Cc5c(F)cccc5F)c4=O)cc3Cl)c2F)C1] 2-(3-chloro-4-((3-fluoro-2-(3- methoxy-3-methylazetidin-1-yl) pyridin-4-yl)oxy)phenyl)-4-(2,6- difluorobenzyl)-2,4-dihydro-3H- 1,2,4-triazol-3-one | White solid, yield: 22.8% | ¹H NMR (400 MHZ, DMSO-d₆) δ 8.40 (s, 1H), 8.16 (dd, J = 11.9, 2.9 Hz, 2H), 7.92 (dd, J = 9.0, 2.6 Hz, 1H), 7.53-7.42 (m, 2H), 7.16 (td, J = 8.1, 4.4 Hz, 2H), 5.71 (d, J = 5.8 Hz, 1H), 5.00 (s, 2H), 3.76 (d, J = 8.6 Hz, 2H), 3.65 (d, J = 8.6 Hz, 2H), 3.13 (s, 3H), 1.38 (s, 3H). | 532.1 |
| Example 498 Method 83 | +++ | 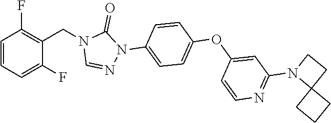 [SMILES: O=c1n(Cc2c(F)cccc2F)cnn1-c1ccc(Oc2ccnc(N3CCC34CCC4)c2)cc1] 2-(4-((2-(1-azaspiro[3.3]heptan- 1-yl)pyridin-4-yl)oxy)phenyl)-4- (2,6-difluorobenzyl)-2,4-dihydro- 3H-1,2,4-triazol-3-one | Off- white solid, yield: 19.8% | ¹H NMR (400 MHZ, DMSO-d₆) δ 8.32 (s, 1H), 7.98 (d, J = 5.8 Hz, 1H), 7.95-7.88 (m, 2H), 7.51-7.42 (m, 1H), 7.26-7.20 (m, 2H), 7.20-7.11 (m, 2H), 6.18 (dd, J = 5.8, 2.2 Hz, 1H), 5.78 (d, J = 2.2 Hz, 1H), 4.98 (s, 2H), 3.67 (t, J = 7.1 Hz, 2H), 2.83 (dt, J = 11.7, 9.2 Hz, 2H), 2.40-2.30 (m, 2H), 2.04-1.89 (m, 2H), 1.76-1.53 (m, 2H). | 476.1 |
| Example 499 Method 83 | +++ | 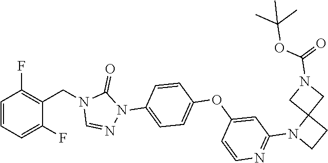 [SMILES: CC(C)(C)OC(=O)N1CC2(CCN2c2cc(Oc3ccc(-n4ncn(Cc5c(F)cccc5F)c4=O)cc3)ccn2)C1] tert-butyl 1-(4-(4-(4-(2,6-difluorobenzyl)- 5-oxo-4,5-dihydro-1H-1,2,4- triazol-1-yl)phenoxy)pyridin-2- yl)-1,6-diazaspiro[3.3]heptane- 6-carboxylate | White solid, yield: 41.2% | ¹H NMR (400 MHZ, DMSO-d₆) δ 8.32 (s, 1H), 7.98 (d, J = 5.8 Hz, 1H), 7.95-7.87 (m, 2H), 7.53-7.46 (m, 1H), 7.28-7.20 (m, 2H), 7.17 (t, J = 8.1 Hz, 2H), 6.33-6.18 (m, 1H), 5.83 (s, 1H), 4.98 (s, 2H), 4.43 (d, J = 9.4 Hz, 2H), 3.88 (d, J = 9.4 Hz, 2H), 3.70 (t, J = 7.0 Hz, 2H), 2.52-2.47 (m, 2H), 1.31 (s, 9H). | 577.2 |
| Example 500 Method 83 | ++++ | 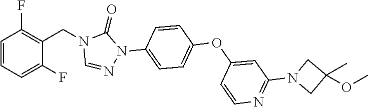 [SMILES: COC1(C)CN(c2cc(Oc3ccc(-n4ncn(Cc5c(F)cccc5F)c4=O)cc3)ccn2)C1] 4-(2,6-difluorobenzyl)-2-(4-((2- (3-methoxy-3-methylazetidin-1- yl)pyridin-4-yl)oxy)phenyl)-2,4- dihydro-3H-1,2,4-triazol-3-one | Light- yellow solid, yield: 17.7% | ¹H NMR (400 MHZ, DMSO-d₆) δ 8.32 (s, 1H), 7.98-7.89 (m, 3H), 7.49 (tt, J = 8.5, 6.7 Hz, 1H), 7.25-7.11 (m, 4H), 6.23 (dd, J = 5.8, 2.2 Hz, 1H), 5.84 (d, J = 2.2 Hz, 1H), 4.99 (s, 2H), 3.80 (d, J = 8.5 Hz, 2H), 3.69 (d, J = 8.6 Hz, 2H), 3.16 (s, 3H), 1.42 (s, 3H). | 480.2 |
| Example 501 Method 83 | ++++ | 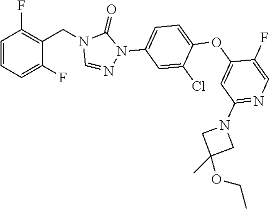 [SMILES: CCOC1(C)CN(c2cc(Oc3ccc(-n4ncn(Cc5c(F)cccc5F)c4=O)cc3Cl)c(F)cn2)C1] 2-(3-chloro-4-((2-(3-ethoxy-3- methylazetidin-1-yl)-5- fluoropyridin-4-yl)oxy)phenyl)- 4-(2,6-difluorobenzyl)-2,4- dihydro-3H-1,2,4-triazol-3-one | Off- white solid, yield: 7.1% | ¹H NMR (400 MHZ, DMSO-d₆) δ 8.40 (s, 1H), 8.16-8.11 (m, 2H), 7.91 (dd, J = 9.0, 2.6 Hz, 1H), 7.52-7.42 (m, 2H), 7.17 (t, J = 8.1 Hz, 2H), 5.69 (d, J = 5.7 Hz, 1H), 4.99 (d, J = 5.6 Hz, 2H), 3.79-3.65 (m, 2H), 3.39-3.31 (m, 2H), 1.40 (s, 3H), 1.10-1.03 (m, 3H). | 546.1 |
| Example 502 Method 83 | ++++ | 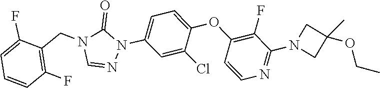 [SMILES: CCOC1(C)CN(c2nccc(Oc3ccc(-n4ncn(Cc5c(F)cccc5F)c4=O)cc3Cl)c2F)C1] 2-(3-chloro-4-((2-(3-ethoxy-3- methylazetidin-1-yl)-3- fluoropyridin-4-yl)oxy)phenyl)- 4-(2,6-difluorobenzyl)-2,4- dihydro-3H-1,2,4-triazol-3-one | White solid, yield: 11.9% | ¹H NMR (400 MHZ, DMSO-d₆) δ 8.38 (s, 1H), 8.11 (d, J = 2.6 Hz, 1H), 7.88 (dd, J = 9.0, 2.6 Hz, 1H), 7.76 (d, J = 5.6 Hz, 1H), 7.45 (dd, J = 24.1, 8.3 Hz, 2H), 7.25-7.09 (m, 2H), 6.15 (t, J = 5.5 Hz, 1H), 4.99 (s, 2H), 4.01 (d, J = 8.6 Hz, 2H), 3.90 (dd, J = 8.7, 2.2 Hz, 2H), 3.39-3.31 (m, 2H), 1.49 (s, 3H), 1.13 (t, J = 7.0 Hz, 3H). | 546.1 |
| Example 503 Method 83 | ++++ | 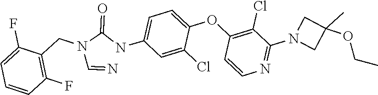 [SMILES: CCOC1(C)CN(c2nccc(Oc3ccc(NC(=O)N(C=N)Cc4c(F)cccc4F)cc3Cl)c2Cl)C1] 2-(3-chloro-4-((3-chloro-2-(3- ethoxy-3-methylazetidin-1-yl) pyridin-4-yl)oxy)phenyl)-4-(2, 6-difluorobenzyl)-2,4-dihydro- 3H-1,2,4-triazol-3-one | White solid, yield: 22.4% | NA | 562.1 |
| Example 504 Method 83 | ++++ | 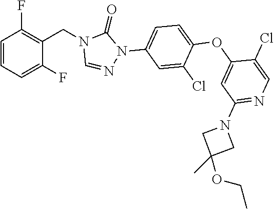 [SMILES: CCOC1(C)CN(c2cc(Oc3ccc(-n4ncn(Cc5c(F)cccc5F)c4=O)cc3Cl)c(Cl)cn2)C1] 2-(3-chloro-4-((5-chloro-2-(3- ethoxy-3-methylazetidin-1-yl) pyridin-4-yl)oxy)phenyl)-4-(2, 6-difluorobenzyl)-2,4-dihydro- 3H-1,2,4-triazol-3-one | White solid, yield: 18.9% | NA | 562.1 |
| Example 505 Method 83 | ++++ | 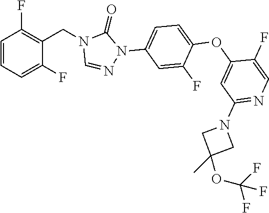 [SMILES: CC1(OC(F)(F)F)CN(c2cc(Oc3ccc(-n4ncn(Cc5c(F)cccc5F)c4=O)cc3F)c(F)cn2)C1] 4-(2,6-difluorobenzyl)-2-(3- fluoro-4-((5-fluoro-2-(3-methyl-3- (trifluoromethoxy)azetidin-1-yl) pyridin-4-yl)oxy)phenyl)-2,4- dihydro-3H-1,2,4-triazol-3-one | White solid, yield: 23.6% | ¹H NMR (400 MHZ, DMSO-d₆) δ 8.40 (s, 1H), 8.17 (d, J = 3.0 Hz, 1H), 7.93 (dd, J = 12.4, 2.5 Hz, 1H), 7.79 (ddd, J = 9.1, 2.6, 1.3 Hz, 1H), 7.48 (td, J = 8.7, 5.6 Hz, 2H), 7.17 (t, J = 8.1 Hz, 2H), 5.90 (d, J = 5.6 Hz, 1H), 5.00 (s, 2H), 3.99 (d, J = 9.2 Hz, 2H), 3.93 (d, J = 9.3 Hz, 2H), 1.68 (d, J = 2.1 Hz, 3H). | 570.1 |
| Example 506 Method 83 | ++++ | 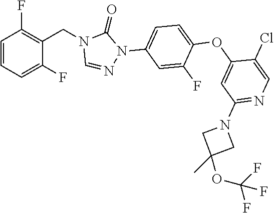 [SMILES: CC1(OC(F)(F)F)CN(c2cc(Oc3ccc(-n4ncn(Cc5c(F)cccc5F)c4=O)cc3F)c(Cl)cn2)C1] 2-(4-((5-chloro-2-(3-methyl-3- (trifluoromethoxy)azetidin-1-yl) pyridin-4-yl)oxy)-3-fluorophenyl)- 4-(2,6-difluorobenzyl)-2,4- dihydro-3H-1,2,4-triazol-3-one | Off- white solid, yield: 23.6% | ¹H NMR (400 MHZ, DMSO-d₆) δ 8.40 (s, 1H), 8.17 (s, 1H), 7.94 (dd, J = 12.4, 2.5 Hz, 1H), 7.80 (dt, J = 8.9, 1.8 Hz, 1H), 7.56-7.45 (m, 2H), 7.23-7.12 (m, 2H), 5.78 (s, 1H), 5.00 (s, 2H), 4.02 (d, J = 9.4 Hz, 2H), 3.94 (d, J = 9.4 Hz, 2H), 1.75-1.57 (s, 3H). | 586.1 |

### Example 507: Method 84

### 2-(4-(2-(3-amino-3-methylazetidin-1-yl)-4-methylthiazole-5-carbonyl)phenyl)-4-(2,6-difluorobenzyl)-2,4-dihydro-3H-1,2,4-triazol-3-one trifluoroacetate

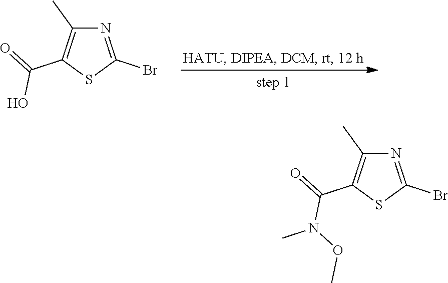

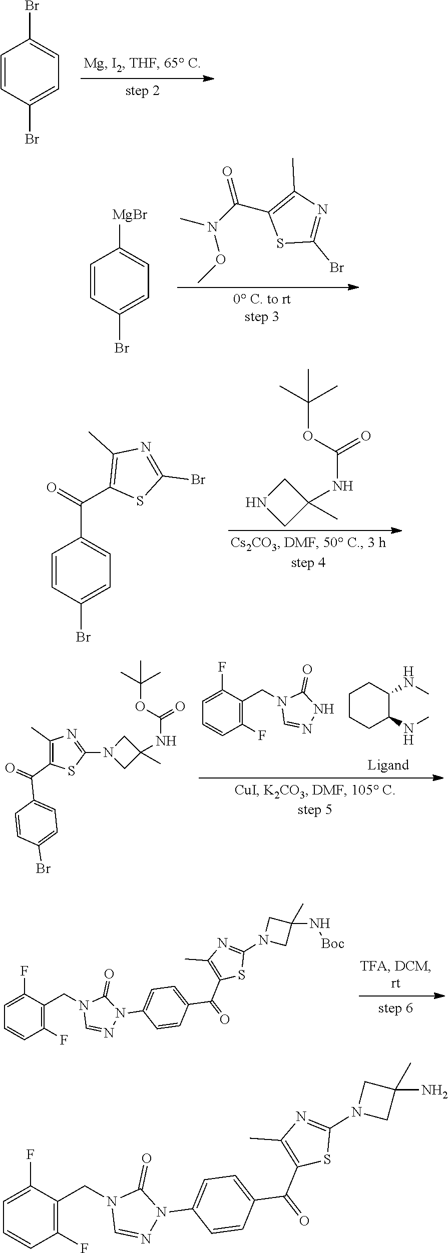

Step 1: To a solution of 2-bromo-4-methyl-thiazole-5-carboxylic acid (1.00 eq, 1.14 g, 5.13 mmol), HATU (1.20 eq, 2.34 g, 6.16 mmol), and N-Methylhydroxylamine hydrochloride (1.00 eq, 0.43 g, 5.13 mmol) in DCM (12 mL), was added DIPEA (2.00 eq, 1.33 g, 10.3 mmol) at room temperature. After addition, the mixture was stirred at 25° C. for 12 h. LCMS indicated the reaction was completed. Then the mixture was added with water (30 mL) and extracted with DCM (30 mL×3). The organics were then combined and dried (Na₂SO₄) before concentration to dryness. The crude was purified by flash chromatography (silica gel column, EtOAc/PE=0 to 50%) to give the product 2-bromo-N-methoxy-N,4-dimethyl-thiazole-5-carboxamide (800 mg, 2.72 mmol, 52.90% yield) as a white solid. MS (m/z): 265.0 [M+H]⁺. ¹H NMR (400 MHz, Chloroform-d) δ 3.72 (s, 4H), 3.33 (s, 3H), 2.76 (s, 3H).

Step 2: To a solution of Mg (1.50 eq, 1.55 g, 63.6 mmol) and iodine (0.0100 eq, 108 mg, 0.424 mmol) in THF (100 mL), was added dropwise 1,4-dibromobenzene (1.00 eq, 10.00 g, 42.4 mmol) in THF (20 mL) at 65° C. under N₂. After addition, the mixture was stirred at 65° C. for 2 h, which mixture was used directly for the next step.

Step 3: To a solution of 2-bromo-N-methoxy-N,4-dimethyl-thiazole-5-carboxamide (1.00 eq, 400 mg, 1.51 mmol) in THF (10 mL) was added dropwise bromo-(4-bromophenyl)magnesium (1.20 eq, 471 mg, 1.81 mmol) in 3 mL THF at 0° C. under N₂. After addition, the mixture was stirred at 25° C. for 12 h. LCMS indicated the reaction was completed. Then the mixture was added with water (30 mL) and extracted with EtOAc (30 mL×3). The organics were then combined and dried (Na₂SO₄) before concentration to dryness. The crude was purified by flash chromatography (Silica gel column, EtOAc/PE=0 to 50%) to give the product (2-bromo-4-methyl-thiazol-5-yl)-(4-bromophenyl)methanone (300 mg, 0.665 mmol, 44.06% yield) as a white solid. MS (m/z): 359.9 [M+H]⁺. ¹H NMR (400 MHz, Chloroform-d) δ 7.66 (d, J=1.8 Hz, 3H), 2.58 (s, 3H).

Step 4: To a solution of (2-bromo-4-methyl-thiazol-5-yl)-(4-bromophenyl)methanone (1.00 eq, 1500 mg, 4.15 mmol), tert-butyl N-(3-methylazetidin-3-yl)carbamate (1.00 eq, 774 mg, 4.15 mmol) in DMF (5 mL), was added Cs₂CO₃ (2.00 eq, 2707 mg, 8.31 mmol) at room temperature. After addition, the mixture was stirred at 50° C. for 3 h. LCMS indicated the reaction was completed. Then the mixture was added with water (20 mL) and extracted with EtOAc (20 mL×3). The organics were then combined and dried (Na₂SO₄) before concentration to dryness. The crude was purified by flash chromatography (Silica gel column, EtOAc/PE=0 to 50%) to give the product tert-butyl (1-(5-(4-bromobenzoyl)-4-methylthiazol-2-yl)-3-methylazetidin-3-yl)carbamate (1200 mg, 2.57 mmol, 61.93% yield) as a yellow solid. MS (m/z): 466.1 [M+H]⁺. ¹H NMR (400 MHz, DMSO-d₆) δ 7.71 (d, J=8.5 Hz, 2H), 7.58 (d, J=8.5 Hz, 2H), 4.18 (d, J=8.7 Hz, 2H), 3.91 (d, J=8.8 Hz, 2H), 2.24 (d, J=2.1 Hz, 3H), 1.49 (s, 3H), 1.39 (s, 9H).

Step 5: To a solution of tert-butyl (1-(5-(4-bromobenzoyl)-4-methylthiazol-2-yl)-3-methylazetidin-3-yl)carbamate (1.00 eq, 100 mg, 0.214 mmol), 4-(2,6-difluorobenzyl)-2,4-dihydro-3H-1,2,4-triazol-3-one (1.20 eq, 54 mg, 0.257 mmol), CuI (0.500 eq, 20 mg, 0.107 mmol), K₂CO₃ (2.00 eq, 59 mg, 0.429 mmol) in 1,4-Dioxane (1 mL), was added (1R,2R)—N1,N2-dimethylcyclohexane-1,2-diamine (0.500 eq, 15 mg, 0.107 mmol) at room temperature under N₂. After addition, the mixture was stirred at 105° C. for 5 h. Then the mixture was cooled and added with water (20 mL) and extracted with EtOAc (20 mL×3). The organics were then combined and dried (Na₂SO₄) before concentration to dryness. The crude was purified by Prep-TLC (EtOAc/PE=1/1) to give the product tert-butyl (1-(5-(4-(4-(2,6-difluorobenzyl)-5-oxo-4,5-dihydro-1H-1,2,4-triazol-1-yl)benzoyl)-4-methylthiazol-2-yl)-3-methylazetidin-3-yl)carbamate (4.0 mg, 0.00610 mmol, 2.85% yield) as a yellow solid. MS (m/z): 597.2 [M+H]⁺. ¹H NMR (400 MHz, Chloroform-d) δ 8.15 (d, J=8.4 Hz, 2H), 7.77 (d, J=8.7 Hz, 2H), 7.64 (s, 1H), 7.41-7.34 (m, 1H), 7.03-6.95 (m, 2H), 4.99 (s, 2H), 4.85-4.62 (m, 4H), 2.44 (s, 3H), 1.63 (s, 3H), 1.45 (s, 9H).

Step 6: To a solution of tert-butyl (1-(5-(4-(4-(2,6-difluorobenzyl)-5-oxo-4,5-dihydro-1H-1,2,4-triazol-1-yl)benzoyl)-4-methylthiazol-2-yl)-3-methylazetidin-3-yl)carbamate (1.00 eq, 10 mg, 0.0176 mmol) in DCM (0.5 mL), was slowly added TFA (50.0 eq, 0.067 mL, 0.879 mmol) at room temperature. After addition, the mixture was stirred for 2 h at 25° C. LCMS showed completion of the reaction. The reaction was concentrated and lyophilized to give 2-(4-(2-(3-amino-3-methylazetidin-1-yl)-4-methylthiazole-5-carbonyl)phenyl)-4-(2,6-difluorobenzyl)-2,4-dihydro-3H-1,2,4-triazol-3-one trifluoroacetate (2.2 mg, 0.00359 mmol, 20.40% yield) as a white solid. MS (m/z): 497.2 [M+H]⁺. ¹H NMR (400 MHz, DMSO-d₆) δ 8.44 (brs, 3H), 8.40 (s, 1H), 8.10-7.98 (m, 2H), 7.80-7.71 (m, 2H), 7.53-7.45 (m, 1H), 7.21-7.11 (m, 2H), 5.00 (s, 2H), 4.18 (d, J=9.6 Hz, 2H), 4.12 (s, 2H), 2.27 (s, 3H), 1.58 (s, 3H).

The synthesis procedure of Example (Compound) 508 was similar to the procedure of Example 507.

| Compound No. | | | Appearance | | MS(m/z) |
|---|---|---|---|---|---|
| Method | Activity | Structure and Name | and Yield | ¹H NMR Data | [M + H]⁺ |
| Example 507 Method 84 | +++ | 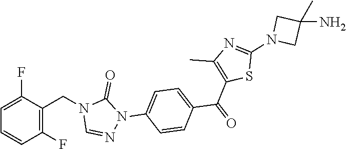 [SMILES: Cc1nc(N2CC(C)(N)C2)sc1C(=O)c1ccc(-n2ncn(Cc3c(F)cccc3F)c2=O)cc1] 2-(4-(2- (3-amino-3-methylazetidin- 1-yl)-4-methylthiazole-5- carbonyl)phenyl)-4-(2,6- difluorobenzyl)-2,4-dihydro- 3H-1,2,4-triazol-3-one | white solid, yield: 20.4% | ¹H NMR (400 MHZ, DMSO-d₆) δ 8.44 (brs, 3H), 8.40 (s, 1H), 8.10-7.98 (m, 2H), 7.80-7.71 (m, 2H), 7.53-7.45 (m, 1H), 7.21-7.11 (m, 2H), 5.00 (s, 2H), 4.18 (d, J = 9.6 Hz, 2H), 4.12 (s, 2H), 2.27 (s, 3H), 1.58 (s, 3H). | 497.2 |
| Example 508 Method 84 | +++ | 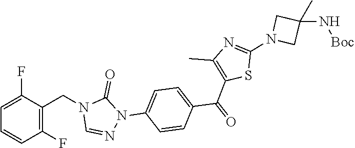 [SMILES: Cc1nc(N2CC(C)(NC(=O)OC(C)(C)C)C2)sc1C(=O)c1ccc(-n2ncn(Cc3c(F)cccc3F)c2=O)cc1] tert-butyl(1- (5-(4-(4-(2,6-difluorobenzyl)- 5-oxo-4,5-dihydro-1H-1,2,4- triazol-1-yl)benzoyl)-4- methylthiazol-2-yl)-3- methylazetidin-3-yl)carbamate | yellow solid, yield: 2.85% | ¹H NMR (400 MHZ, Chloroform-d) δ 8.15 (d, J = 8.4 Hz, 2H), 7.77 (d, J = 8.7 Hz, 2H), 7.64 (s, 1H), 7.41-7.34 (m, 1H), 7.03-6.95 (m, 2H), 4.99 (s, 2H), 4.85-4.62 (m, 4H), 2.44 (s, 3H), 1.63 (s, 3H), 1.45 (s, 9H). | 597.2 |

### Example 509: Method 85

### 2-(4-((2-(3-amino-3-methylazetidin-1-yl)-4-methylthiazol-5-yl)methyl)phenyl)-4-(2,6-difluorobenzyl)-2,4-dihydro-3H-1,2,4-triazol-3-one

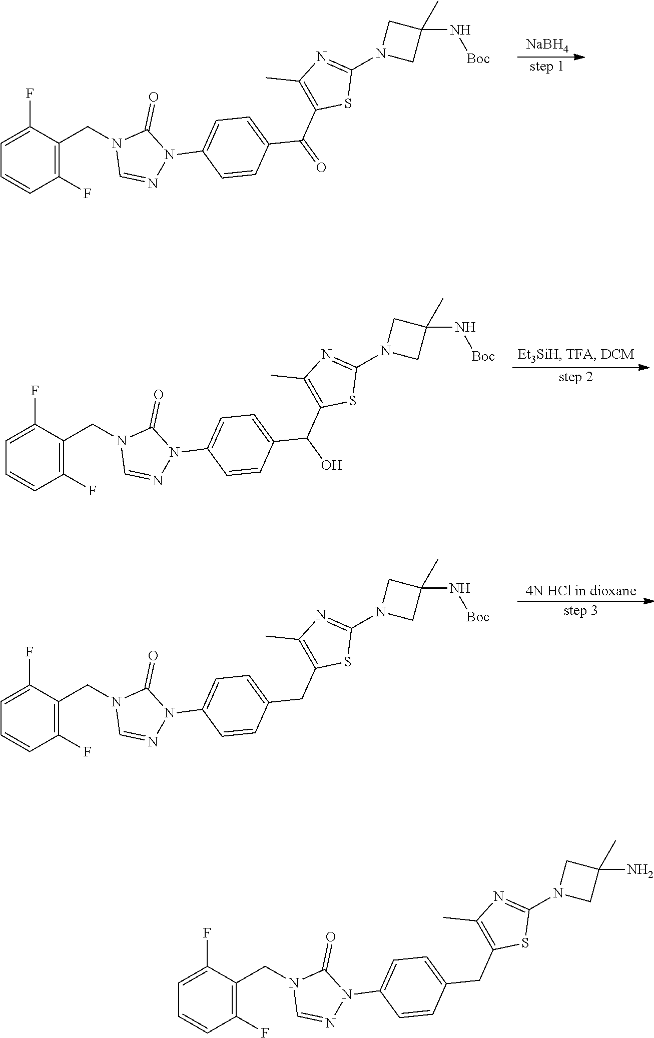

Step 1: To a solution of tert-butyl (1-(5-(4-(4-(2,6-difluorobenzyl)-5-oxo-4,5-dihydro-1H-1,2,4-triazol-1-yl)benzoyl)-4-methylthiazol-2-yl)-3-methylazetidin-3-yl)carbamate (1.00 eq, 100 mg, 0.134 mmol) in THF (1 mL) and water (1 mL), was added NaBH₄ (2.00 eq, 10 mg, 0.268 mmol) at 0° C. After addition, the mixture was stirred at 0° C. for 2 h. LCMS indicated the reaction was completed. Then the mixture was added with water (20 mL) and extracted with EtOAc (20 mL×3). The organics were then combined and dried (Na₂SO₄) before concentration to dryness. The crude was purified by flash chromatography (silica gel column, 10 g, EtOAc/PE, 0 to 100%) to give the product tert-butyl (1-(5-((4-(4-(2,6-difluorobenzyl)-5-oxo-4,5-dihydro-1H-1,2,4-triazol-1-yl)phenyl)(hydroxy)methyl)-4-methylthiazol-2-yl)-3-methylazetidin-3- yl)carbamate (60 mg, 0.0902 mmol, 67.27% yield) as a yellow gum. MS (m/z): 599.2 [M+H]⁺.

Step 2: To a stirred solution of tert-butyl (1-(5-((4-(4-(2,6-difluorobenzyl)-5-oxo-4,5-dihydro-1H-1,2,4-triazol-1-yl)phenyl)(hydroxy)methyl)-4-methylthiazol-2-yl)-3-methylazetidin-3-yl)carbamate (1.00 eq, 100 mg, 0.167 mmol), in DCM (1 mL) was added triethylsilane (10.0 eq, 194 mg, 1.67 mmol) at room temperature. After addition, the mixture was stirred at 25° C. for 2 h. LCMS indicated the reaction was completed. Then the mixture was added with water (20 mL) and extracted with DCM (20 mL×3). The organics were then combined and dried (Na₂SO₄) before concentration to dryness. The crude was purified with Prep-TLC (50% EtOAc in PE) to give the product tert-butyl (1-(5-(4-(4-(2,6-difluorobenzyl)-5-oxo-4,5-dihydro-1H-1,2,4-triazol-1-yl)benzyl)-4-methylthiazol-2-yl)-3-methylazetidin-3-yl)carbamate (50 mg, 0.0858 mmol, 51.37% yield) as a yellow solid. MS (m/z): 583.2 [M+H]⁺. ¹H NMR (400 MHz, DMSO-d₆) δ 8.27 (s, 1H), 7.76 (d, J=8.6 Hz, 2H), 7.52-7.44 (m, 1H), 7.39 (brs, 1H), 7.25 (d, J=8.6 Hz, 2H), 7.16 (t, J=8.1 Hz, 2H), 4.96 (s, 2H), 3.98-3.86 (m, 4H), 3.68 (d, J=7.8 Hz, 2H), 2.13 (s, 3H), 1.45 (s, 3H), 1.37 (s, 9H).

Step 3: A solution of tert-butyl (1-(5-(4-(4-(2,6-difluorobenzyl)-5-oxo-4,5-dihydro-1H-1,2,4-triazol-1-yl)benzyl)-4-methylthiazol-2-yl)-3-methylazetidin-3-yl)carbamate (1.00 eq, 30 mg, 0.0515 mmol) in 4N HCl in dioxane (0.50 mL) was stirred for 2 h at 25° C. LCMS showed completion. The reaction was concentrated and lyophilized to give the product (13 mg, 0.0238 mmol, 46.22% yield) as a white solid. MS (m/z): 483.2 [M+H]⁺. ¹H NMR (400 MHz, DMSO-d₆) δ 8.72 (s, 3H), 8.30 (s, 1H), 7.79 (d, J=8.6 Hz, 2H), 7.52-7.44 (m, 1H), 7.30 (d, J=8.6 Hz, 2H), 7.16 (t, J=8.1 Hz, 2H), 4.97 (s, 2H), 4.23 (d, J=9.6 Hz, 2H), 4.08 (d, J=9.6 Hz, 2H), 3.99 (s, 2H), 2.24 (s, 3H), 1.57 (s, 3H).

The synthesis procedure of Examples (Compounds) 510-512 was similar to the procedure of Example 509.

| Compound No. | | | Appearance | | MS(m/z) |
|---|---|---|---|---|---|
| Method | Activity | Structure and Name | and Yield | ¹H NMR Data | [M + H]⁺ |
| Example 509 Method 85 | ++++ | 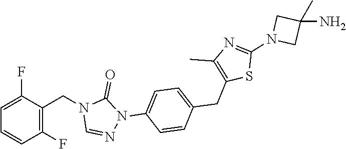 [SMILES: Cc1nc(N2CC(C)(N)C2)sc1Cc1ccc(-n2ncn(Cc3c(F)cccc3F)c2=O)cc1] 2-(4-((2-(3-amino-3- methylazetidin-1-yl)-4- methylthiazol-5-yl)methyl) phenyl)-4-(2,6-difluorobenzyl)- 2,4-dihydro-3H-1,2,4-triazol- 3-one | white solid, yield: 46.22% | ¹H NMR (400 MHZ, DMSO-d₆) δ 8.72 (s, 3H), 8.30 (s, 1H), 7.79 (d, J = 8.6 Hz, 2H), 7.52-7.44 (m, 1H), 7.30 (d, J = 8.6 Hz, 2H), 7.16 (t, J = 8.1 Hz, 2H), 4.97 (s, 2H), 4.23 (d, J = 9.6 Hz, 2H), 4.08 (d, J = 9.6 Hz, 2H), 3.99 (s, 2H), 2.24 (s, 3H), 1.57 (s, 3H). | 483.2 |
| Example 510 Method 85 | +++ | 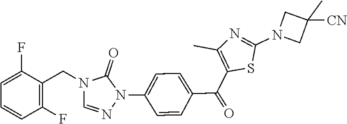 [SMILES: Cc1nc(N2CC(C)(C#N)C2)sc1C(=O)c1ccc(-n2ncn(Cc3c(F)cccc3F)c2=O)cc1] 1-(5-(4-(4-(2,6-difluorobenzyl)- 5-oxo-4,5-dihydro-1H-1,2,4- triazol-1-yl)benzoyl)-4- methylthiazol-2-yl)-3- methylazetidine-3-carbonitrile | yellow solid, yield: 44.57% | ¹H NMR (400 MHZ, DMSO-d₆) δ 8.39 (s, 1H), 8.03 (d, J = 8.8 Hz, 2H), 7.76 (d, J = 8.7 Hz, 2H), 7.57-7.42 (m, 1H), 7.17 (t, J = 8.1 Hz, 2H), 5.00 (s, 2H), 4.45 (d, J = 8.8 Hz, 2H), 4.09 (d, J = 8.8 Hz, 2H), 2.26 (s, 3H), 1.68 (s, 3H). | 507.1 |
| Example 511 Method 85 | ++ | 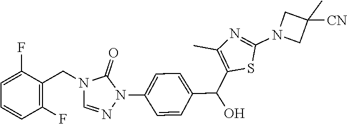 [SMILES: Cc1nc(N2CC(C)(C#N)C2)sc1C(O)c1ccc(-n2ncn(Cc3c(F)cccc3F)c2=O)cc1] 1-(5-((4-(4-(2,6-difluorobenzyl)- 5-oxo-4,5-dihydro-1H-1,2,4- triazol-1-yl)phenyl)(hydroxy) methyl)-4-methylthiazol-2-yl)- 3-methylazetidine-3-carbonitrile | yellow gum, yield: 61.63% | ¹H NMR (400 MHZ, DMSO-d₆) δ 8.28 (s, 1H), 7.79 (d, J = 8.7 Hz, 2H), 7.52-7.44 (m, 1H), 7.44-7.37 (m, 2H), 7.16 (t, J = 8.1 Hz, 2H), 6.07 (d, J = 3.7 Hz, 1H), 5.88 (d, J = 3.7 Hz, 1H), 4.97 (s, 2H), 4.19 (dd, J = 8.0, 1.9 Hz, 2H), 3.87 (dd, J = 8.1, 6.6 Hz, 2H), 2.18 (s, 3H), 1.60 (s, 3H). | 509.1 |
| Example 512 Method 85 | ++++ | 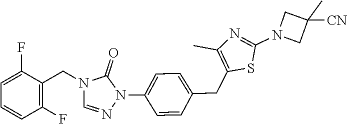 [SMILES: Cc1nc(N2CC(C)(C#N)C2)sc1Cc1ccc(-n2ncn(Cc3c(F)cccc3F)c2=O)cc1] 1-(5-(4-(4-(2,6-difluorobenzyl)- 5-oxo-4,5-dihydro-1H-1,2,4- triazol-1-yl)benzyl)-4- methylthiazol-2-yl)-3- methylazetidine-3-carbonitrile | white solid, yield: 16.02% | ¹H NMR (400 MHZ, DMSO-d₆) δ 8.28 (s, 1H), 7.77 (d, J = 8.7 Hz, 2H), 7.52-7.44 (m, 1H), 7.26 (d, J = 8.7 Hz, 2H), 7.16 (t, J = 8.1 Hz, 2H), 4.96 (s, 2H), 4.20 (d, J = 8.0 Hz, 2H), 3.94 (s, 2H), 3.88 (d, J = 8.0 Hz, 2H), 2.15 (s, 3H), 1.61 (s, 3H). | 493.2 |

### Example 513: Method 86

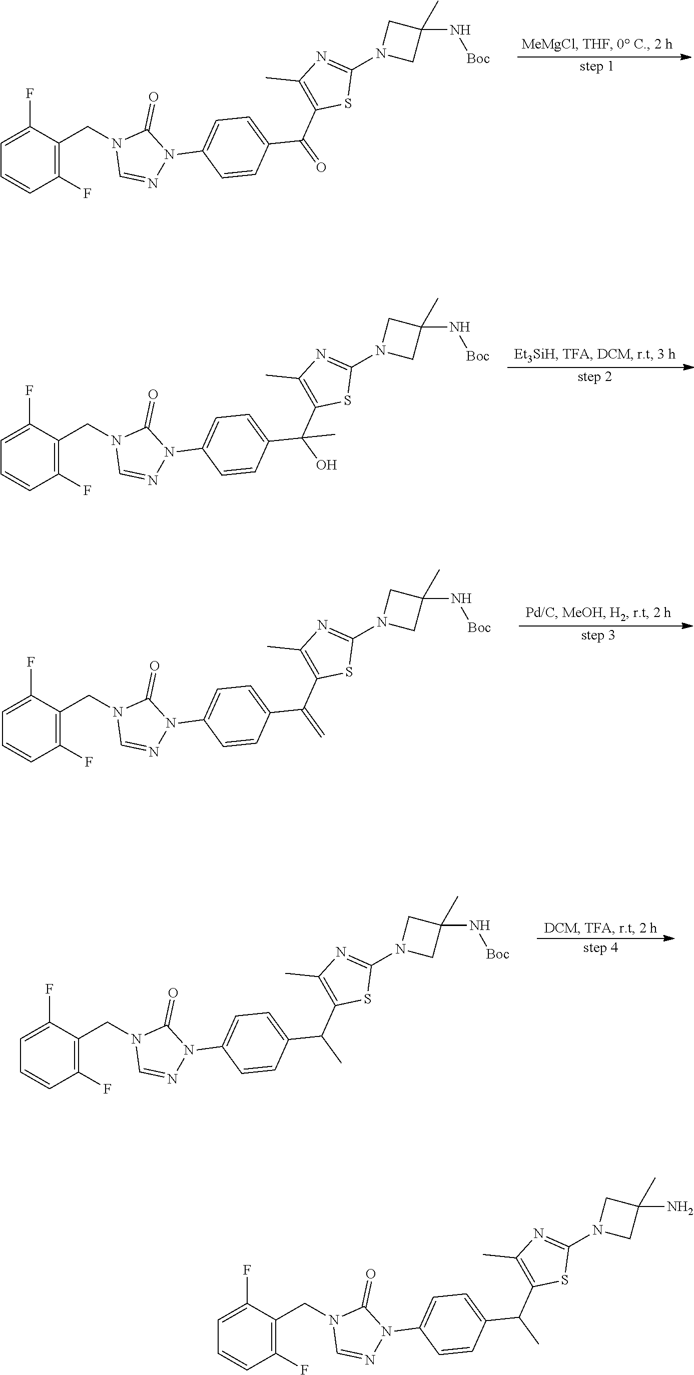

Step 1: To a solution of tert-butyl N-[1-[5-[4-[4-[(2,6-difluorophenyl)methyl]-5-oxo-1,2,4-triazol-1-yl]benzoyl]-4-methyl-thiazol-2-yl]-3-methyl-azetidin-3-yl]carbamate (1.00 eq, 150 mg, 0.239 mmol) in THF (10 mL), was added dropwise MeMgCl (6.00 eq, 0.48 mL, 1.43 mmol) at 0° C. under N₂. After addition, the mixture was stirred at 0° C. for 2 h. LCMS indicated the reaction was completed. Then the mixture was added with water (20 mL) and extracted with EtOAc (20 mL×3). The organics were then combined and dried (Na₂SO₄) before concentration to dryness. The crude was purified with Prep-TLC (50% EtOAc in PE) to give the product tert-butyl N-[1-[5-[1-[4-[4-[(2,6-difluorophenyl)methyl]-5-oxo-1,2,4-triazol-1-yl]phenyl]-1-hydroxy-ethyl]-4-methyl-thiazol-2-yl]-3-methyl-azetidin-3-yl]carb amate (100 mg, 0.147 mmol, 61.50% yield), as a white solid. MS (m/z): 613.2 [M+H]⁺. ¹H NMR (400 MHz, DMSO-d₆) δ 8.28 (s, 1H), 7.78 (d, J=8.8 Hz, 2H), 7.51-7.45 (m, 3H), 7.16 (t, J=8.1 Hz, 2H), 5.94 (s, 1H), 4.97 (s, 2H), 3.96 (d, J=7.9 Hz, 2H), 3.70 (d, J=7.8 Hz, 2H), 1.77 (d, J=2.4 Hz, 6H), 1.47 (s, 3H), 1.38 (s, 9H).

Step 2: To a solution of tert-butyl N-[1-[5-[1-[4-[4-[(2,6-difluorophenyl)methyl]-5-oxo-1,2,4-triazol-1-yl]phenyl]-1-hydroxy-ethyl]-4-methyl-thiazol-2-yl]-3-methyl-azetidin-3-yl]carbamate (1.00 eq, 100 mg, 0.163 mmol) in DCM (5 mL), were added triethylsilane (10.0 eq, 190 mg, 1.63 mmol) and TFA (10.0 eq, 0.12 mL, 1.63 mmol) at room temperature. After addition, the mixture was stirred at 25° C. for 3 h. LCMS indicated the reaction was completed. Then the reaction mixture was concentrated to dryness. The crude was purified with Prep-TLC (10% MeOH in DCM) to give the product tert-butyl N-[1-[5-[1-[4-[4-[(2,6-difluorophenyl)methyl]-5-oxo-1,2,4-triazol-1-yl]phenyl]vinyl]-4-methyl-thiazol-2-yl]-3-methyl-azetidin-3-yl]carbamate (55 mg, 0.0832 mmol, 51.0% yield) as a white solid. MS (m/z): 595.2 [M+H]⁺. ¹H NMR (400 MHz, DMSO-d₆) δ 8.32 (s, 1H), 7.87 (d, J=8.8 Hz, 2H), 7.52-7.44 (m, 2H), 7.42 (d, J=8.8 Hz, 2H), 7.16 (t, J=8.1 Hz, 2H), 5.54 (s, 1H), 5.27 (s, 1H), 4.98 (s, 2H), 4.03 (d, J=8.0 Hz, 2H), 3.77 (d, J=8.0 Hz, 2H), 1.93 (s, 3H), 1.49 (s, 3H), 1.39 (s, 9H).

Step 3: To a solution of tert-butyl N-[1-[5-[1-[4-[4-[(2,6-difluorophenyl)methyl]-5-oxo-1,2,4-triazol-1-yl]phenyl]vinyl]-4-methyl-thiazol-2-yl]-3-methyl-azetidin-3-yl]carbamat e (1.00 eq, 50 mg, 0.0841 mmol) in methanol (2 mL), was added Pd/C (10 mg) at room temperature. After addition, the mixture was stirred at 25° C. for 2 h under H₂ atmosphere. LCMS indicated the reaction was completed. Then the reaction mixture was filtered and concentrated to dryness. The crude was purified with Prep-TLC (50% EtOAc in PE) to give the product tert-butyl N-[1-[5-[1-[4-[4-[(2,6-difluorophenyl)methyl]-5-oxo-1,2,4-triazol-1-yl]phenyl]ethyl]-4-methyl-thiazol-2-yl]-3-methyl-azetidin-3-yl]carbamat e (25 mg, 0.0398 mmol, 47.34% yield), as a white solid. MS (m/z): 597.2 [M+H]⁺. ¹H NMR (400 MHz, DMSO-d₆) δ 8.27 (s, 1H), 7.76 (d, J=8.5 Hz, 2H), 7.54-7.42 (m, 2H), 7.33 (d, J=8.4 Hz, 2H), 7.16 (t, J=8.0 Hz, 2H), 4.96 (s, 2H), 4.32 (q, J=7.0 Hz, 1H), 3.94 (d, J=7.9 Hz, 2H), 3.69 (d, J=7.8 Hz, 2H), 2.07 (s, 3H), 1.50 (d, J=7.1 Hz, 3H), 1.45 (s, 3H), 1.37 (s, 9H).

Step 4: To a solution of tert-butyl N-[1-[5-[1-[4-[4-[(2,6-difluorophenyl)methyl]-5-oxo-1,2,4-triazol-1-yl]phenyl]ethyl]-4-methyl-thiazol-2-yl]-3-methyl-azetidin-3-yl]carbamat e (1.00 eq, 21 mg, 0.0352 mmol) in DCM (1 mL), was added TFA (100 eq, 0.27 mL, 3.52 mmol) at room temperature. After addition, the mixture was stirred at 25° C. for 2 h. LCMS indicated the reaction was completed. Then the reaction mixture was concentrated to dryness and lyophilized to give the product 2-[4-[1-[2-(3-amino-3-methyl-azetidin-1-yl)-4-methyl-thiazol-5-yl]ethyl]phenyl]-4-[(2,6-difluorophenyl)methyl]-1,2,4-triazol-3-one; 2,2,2-trifluoroacetic acid (20 mg, 0.0324 mmol, 92.14% yield), as a white solid. MS (m/z): 497.2 [M+H]⁺. ¹H NMR (400 MHz, DMSO-d₆) δ 8.33 (s, 3H), 8.29 (s, 1H), 7.77 (d, J=8.6 Hz, 2H), 7.52-7.44 (m, 1H), 7.34 (d, J=8.7 Hz, 2H), 7.20-7.11 (m, 2H), 4.96 (s, 2H), 4.40-4.35 (m, 1H), 4.02-3.98 (m, 2H), 3.92-3.89 (m, 2H), 2.13 (s, 3H), 1.54 (s, 3H), 1.52 (d, J=7.1 Hz, 3H).

The synthesis procedure of Examples (Compounds) 514-516 was similar to the procedure of Example 513.

| Compound No. | | | Appearance | | MS(m/z) |
|---|---|---|---|---|---|
| Method | Activity | Structure and Name | and Yield | ¹H NMR Data | [M + H]⁺ |
| Example 513 Method 86 | +++ | 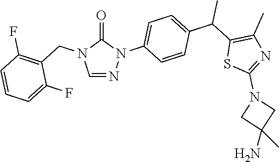 [SMILES: Cc1nc(N2CC(C)(N)C2)sc1C(C)c1ccc(-n2ncn(Cc3c(F)cccc3F)c2=O)cc1] 2-(4-(1- (2-(3-amino-3-methylazetidin- 1-yl)-4-methylthiazol-5-yl)ethyl) phenyl)-4-(2,6-difluorobenzyl)- 2,4-dihydro-3H-1,2,4-triazol-3- one | white solid, yield: 92.14% | ¹H NMR (400 MHZ, DMSO-d₆) δ 8.33 (s, 3H), 8.29 (s, 1H), 7.77 (d, J = 8.6 Hz, 2H), 7.52-7.44 (m, 1H), 7.34 (d, J = 8.7 Hz, 2H), 7.20-7.11 (m, 2H), 4.96 (s, 2H), 4.40-4.35 (m, 1H), 4.02-3.98 (m, 2H), 3.92-3.89 (m, 2H), 2.13 (s, 3H), 1.54 (s, 3H), 1.52 (d, J = 7.1 Hz, 3H). | 497.2 |
| Example 514 Method 86 | ++++ | 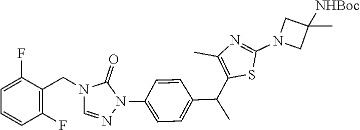 [SMILES: Cc1nc(N2CC(C)(NC(=O)OC(C)(C)C)C2)sc1C(C)c1ccc(-n2ncn(Cc3c(F)cccc3F)c2=O)cc1] tert-butyl (1-(5-(1-(4-(4-(2,6-difluorobenzyl)- 5-oxo-4,5-dihydro-1H-1,2,4-triazol- 1-yl)phenyl)ethyl)-4-methylthiazol- 2-yl)-3-methylazetidin-3-yl) carbamate | white solid, yield: 47.34% | ¹H NMR (400 MHZ, DMSO-d₆) δ 8.27 (s, 1H), 7.76 (d, J = 8.5 Hz, 2H), 7.54-7.42 (m, 2H), 7.33 (d, J = 8.4 Hz, 2H), 7.16 (t, J = 8.0 Hz, 2H), 4.96 (s, 2H), 4.32 (q, J = 7.0 Hz, 1H), 3.94 (d, J = 7.9 Hz, 2H), 3.69 (d, J = 7.8 Hz, 2H), 2.07 (s, 3H), 1.50 (d, J = 7.1 Hz, 3H), 1.45 (s, 3H), 1.37 (s, 9H). | 597.2 |
| Example 515 Method 86 | ++++ | 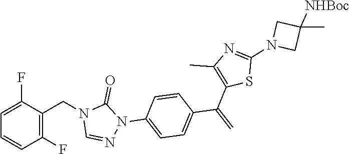 [SMILES: C=C(c1ccc(-n2ncn(Cc3c(F)cccc3F)c2=O)cc1)c1sc(N2CC(C)(NC(=O)OC(C)(C)C)C2)nc1C] 1-(5-((4-(4-(2,6-difluorobenzyl)-5- oxo-4,5-dihydro-1H-1,2,4-triazol- 1-yl)phenyl)(hydroxy)methyl)-4- methylthiazol-2-yl)-3- methylazetidine-3-carbonitrile | white solid, yield: 51.0% | ¹H NMR (400 MHZ, DMSO-d₆) δ 8.32 (s, 1H), 7.87 (d, J = 8.8 Hz, 2H), 7.52-7.44 (m, 2H), 7.42 (d, J = 8.8 Hz, 2H), 7.16 (t, J = 8.1 Hz, 2H), 5.54 (s, 1H), 5.27 (s, 1H), 4.98 (s, 2H), 4.03 (d, J = 8.0 Hz, 2H), 3.77 (d, J = 8.0 Hz, 2H), 1.93 (s, 3H), 1.49 (s, 3H), 1.39 (s, 9H). | 595.2 |
| Example 516 Method 86 | ++++ | 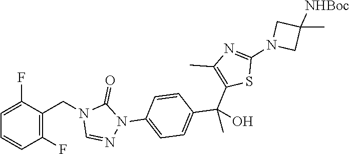 [SMILES: Cc1nc(N2CC(C)(NC(=O)OC(C)(C)C)C2)sc1C(C)(O)c1ccc(-n2ncn(Cc3c(F)cccc3F)c2=O)cc1] tert-butyl (1-(5-(1-(4-(4-(2,6-difluorobenzyl)- 5-oxo-4,5-dihydro-1H-1,2,4-triazol- 1-yl)phenyl)-1-hydroxyethyl)- 4-methylthiazol-2-yl)-3- methylazetidin-3-yl)carbamate | white solid, yield: 61.50% | ¹H NMR (400 MHZ, DMSO-d₆) δ 8.28 (s, 1H), 7.78 (d, J = 8.8 Hz, 2H), 7.51-7.45 (m, 3H), 7.16 (t, J = 8.1 Hz, 2H), 5.94 (s, 1H), 4.97 (s, 2H), 3.96 (d, J = 7.9 Hz, 2H), 3.70 (d, J = 7.8 Hz, 2H), 1.77 (d, J = 2.4 Hz, 6H), 1.47 (s, 3H), 1.38 (s, 9H). | 613.2 |

### Example 517: Method 87

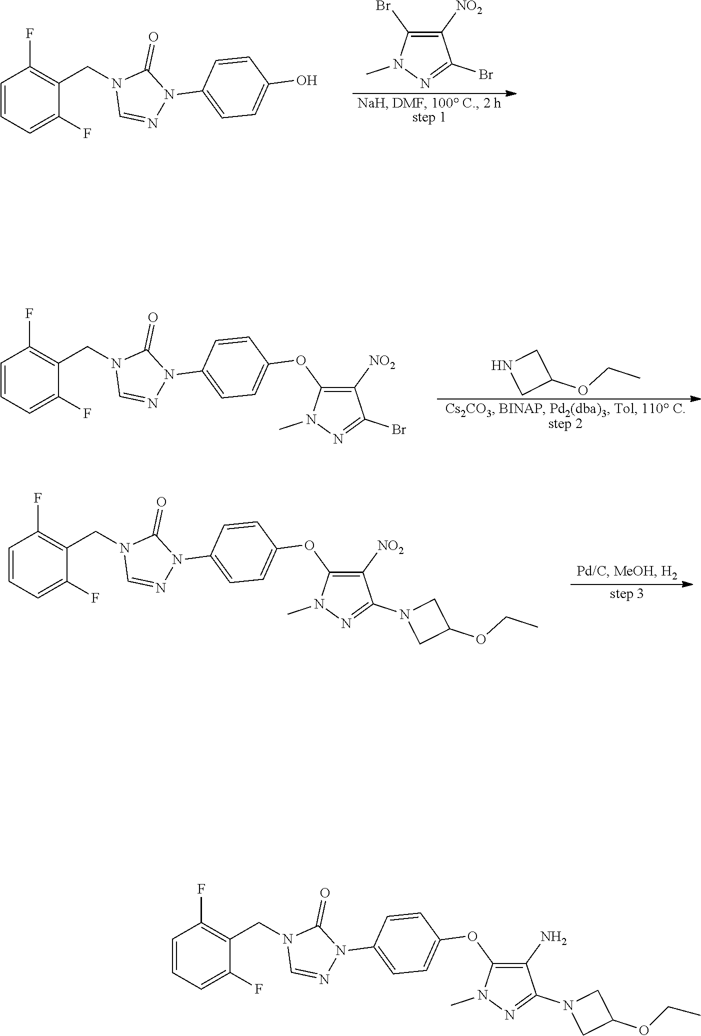

Step 1: To a solution of 4-[(2,6-difluorophenyl)methyl]-2-(4-hydroxyphenyl)-1,2,4-triazol-3-one (1.00 eq, 100 mg, 0.330 mmol), and 3,5-dibromo-1-methyl-4-nitro-pyrazole (1.10 eq, 103 mg, 0.363 mmol) in DMF (2 mL), was added NaH (1.20 eq, 16 mg, 0.396 mmol) at room temperature under N₂. After addition, the mixture was stirred at 100° C. for 2 h. Then the mixture was added with water (20 mL) and extracted with EtOAc (10 mL×3). The organics were then combined and dried (Na₂SO₄) before concentration to dryness. The crude 2-[4-(5-bromo-2-methyl-4-nitro-pyrazol-3-yl)oxyphenyl]-4-[(2,6-difluorophenyl)methyl]-1,2,4-triazol-3-one (100 mg, 0.118 mmol, 35.87% yield) was used for next step directly. MS (m/z): 507.0 [M+H]⁺.

Step 2: To a solution of 2-[4-(5-bromo-2-methyl-4-nitro-pyrazol-3-yl)oxyphenyl]-4-[(2,6-difluorophenyl)methyl]-1,2,4-triazol-3-one (1.00 eq, 100 mg, 0.197 mmol), 3-ethoxyazetidine (1.00 eq, 20 mg, 0.197 mmol), Cs₂CO₃ (3.00 eq, 193 mg, 0.591 mmol), and BINAP (0.400 eq, 49 mg, 0.0789 mmol) in toluene (1 mL), was added at Pd₂(dba)₃ (0.200 eq, 36 mg, 0.0394 mmol) room temperature under N₂. After addition, the mixture was stirred at 110° C. for 2 h. Then the mixture was added with water (20 mL) and extracted with EtOAc (10 mL×3). The organics were then combined and dried (Na₂SO₄) before concentration to dryness. The crude was purified with prep-TLC (PE/EtOAc=1:1) to give the product 4-[(2,6-difluorophenyl)methyl]-2-[4-[5-(3-ethoxyazetidin-1-yl)-2-methyl-4-nitro-pyrazol-3-yl]oxyphenyl]-1,2,4-triazol-3-one (17 mg,0.0290 mmol, 14.71% yield) as a white solid. MS (m/z): 528.2 [M+H]⁺. ¹H NMR (400 MHz, DMSO-d₆) δ 8.30 (s, 1H), 7.86 (d, J=9.1 Hz, 2H), 7.53-7.45 (m, 1H), 7.16 (t, J=8.1 Hz, 2H), 7.10-7.02 (m, 2H), 4.97 (s, 2H), 4.35 (t, J=5.7 Hz, 1H), 4.08 (dd, J=8.1, 6.2 Hz, 2H), 3.71 (dd, J=8.2, 5.0 Hz, 2H), 3.43-3.40 (m, 2H), 3.37 (s, 3H), 1.13 (t, J=7.0 Hz, 3H).

Step 3: To a solution of 4-[(2,6-difluorophenyl)methyl]-2-[4-[5-(3-ethoxyazetidin-1-yl)-2-methyl-4-nitro-pyrazol-3-yl]oxyphenyl]-1,2,4-triazol-3-one (1.00 eq, 17 mg, 0.0322 mmol) in Methanol (2 mL), was added at Pd/C (3.0 mg) room temperature under H₂. After addition, the mixture was stirred at room temperature for 12 h. Then it was evaporated to dryness. The crude was purified with prep-HPLC (0.1% TFA as additive) to give the product 2-[4-[4-amino-5-(3-ethoxyazetidin-1-yl)-2-methyl-pyrazol-3-yl]oxyphenyl]-4-[(2,6-difluorophenyl)methyl]-1,2,4-triazol-3-one (5.4 mg, 33.68% yield) as a pink solid. MS (m/z): 498.2 [M+H]⁺. ¹H NMR (400 MHz, DMSO-d₆) δ 8.30 (s, 1H), 7.86 (d, J=9.1 Hz, 2H), 7.53-7.45 (m, 1H), 7.16 (t, J=8.1 Hz, 2H), 7.10-7.02 (m, 2H), 4.97 (s, 2H), 4.35 (t, J=5.7 Hz, 1H), 4.08 (dd, J=8.1, 6.2 Hz, 2H), 3.71 (dd, J=8.2, 5.0 Hz, 2H), 3.43-3.40 (m, 2H), 3.37 (s, 3H), 1.13 (t, J=7.0 Hz, 3H).

| Com- | | | | | |
|---|---|---|---|---|---|
| pound | | | Appearance | | |
| No. | | | and | | MS(m/z) |
| Method | Activity | Structure and Name | Yield | ¹H NMR Data | [M + H]⁺ |
| Example 517 Method 87 | ++ | 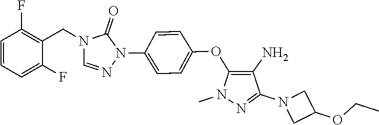 [SMILES: CCOC1CN(c2nn(C)c(Oc3ccc(-n4ncn(Cc5c(F)cccc5F)c4=O)cc3)c2N)C1] 2-(4-((4- amino-3-(3-ethoxyazetidin- 1-yl)-1-methyl-1H-pyrazol- 5-yl)oxy)phenyl)-4-(2,6- difluorobenzyl)-2,4-dihydro- 3H-1,2,4-triazol-3-one | pink solid, yield: 33.68% | ¹H NMR (400 MHZ, DMSO-d₆) δ 8.30 (s, 1H), 7.86 (d, J = 9.1 Hz, 2H), 7.53-7.45 (m, 1H), 7.16 (t, J = 8.1 Hz, 2H), 7.10-7.02 (m, 2H), 4.97 (s, 2H), 4.35 (t, J = 5.7 Hz, 1H), 4.08 (dd, J = 8.1, 6.2 Hz, 2H), 3.71 (dd, J = 8.2, 5.0 Hz, 2H), 3.43-3.40 (m, 2H), 3.37 (s, 3H), 1.13 (t, J = 7.0 Hz, 3H). | 498.2 |

### Example 518: Method 88

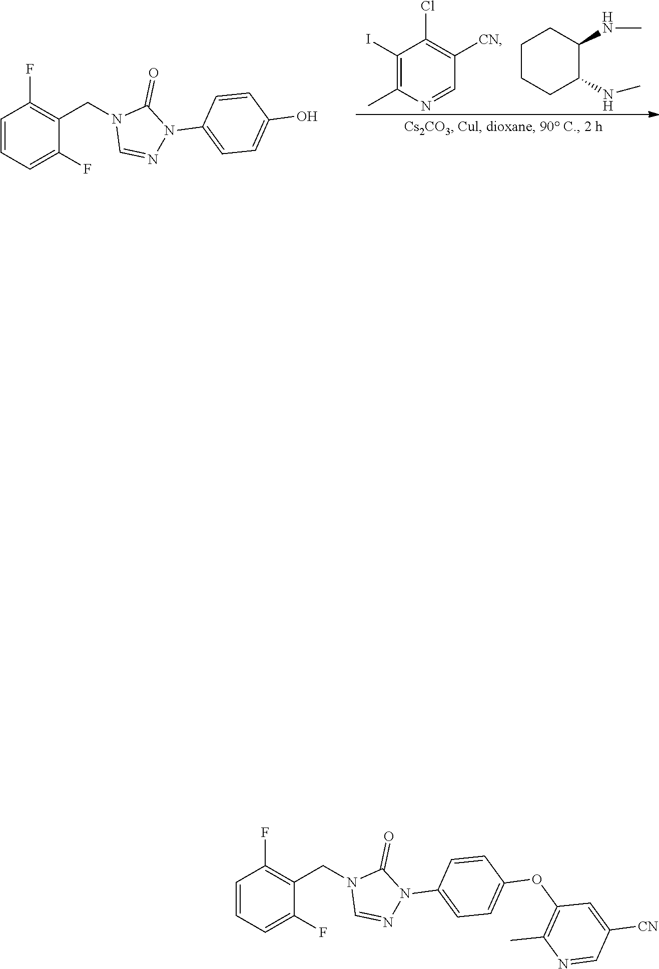

To a solution 4-[(2,6-difluorophenyl)methyl]-2-(4-hydroxyphenyl)-1,2,4-triazol-3-one (1.00 eq, 100 mg, 0.330 mmol), 4-chloro-5-iodo-6-methyl-pyridine-3-carbonitrile (1.00 eq, 92 mg, 0.330 mmol), copper(I) iodide (0.500 eq, 31 mg, 0.165 mmol), and (1R,2R)—N,N′-Dimethyl-1,2-cyclohexanediamine (0.500 eq, 23 mg, 0.165 mmol) in 1,4-Dioxane (3 mL), was added Cs₂CO₃ (2.00 eq, 215 mg, 0.660 mmol) at room temperature under N₂. After addition, the mixture was stirred at 90° C. for 2 h. Then the mixture was added with water (20 mL) and extracted with DCM (10 mL×3). The organics were then combined and dried (Na₂SO₄) before concentration to dryness. The crude was purified with prep-HPLC to give the product (33.3 mg, 24.08% yield) as a white solid. MS (m/z): 420.1 [M+H]⁺. ¹H NMR (400 MHz, DMSO-d₆) δ 8.84 (s, 1H), 8.36 (s, 1H), 8.05-7.96 (m, 2H), 7.55-7.43 (m, 1H), 7.43-7.36 (m, 2H), 7.17 (t, J=8.1 Hz, 2H), 6.70 (s, 1H), 5.00 (s, 2H), 2.42 (s, 3H).

| Compound No. | | | Appearance | | MS(m/z) |
|---|---|---|---|---|---|
| Method | Activity | Structure and Name | and Yield | ¹H NMR Data | [M + H]⁺ |
| Example 518 Method 88 | ++ | 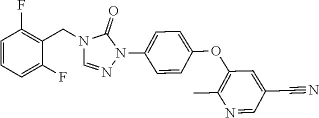 [SMILES: Cc1ncc(C#N)cc1Oc1ccc(-n2ncn(Cc3c(F)cccc3F)c2=O)cc1] 5-(4-(4-(2,6-difluorobenzyl)-5- oxo-4,5-dihydro-1H-1,2,4- triazol-1-yl)phenoxy)-6- methylnicotinonitrile | white solid, yield: 24.08% | ¹H NMR (400 MHZ, DMSO-d₆) δ 8.84 (s, 1H), 8.36 (s, 1H), 8.05-7.96 (m, 2H), 7.55-7.43 (m, 1H), 7.43-7.36 (m, 2H), 7.17 (t, J = 8.1 Hz, 2H), 6.70 (s, 1H), 5.00 (s, 2H), 2.42 (s, 3H). | 420.1 |

### Example 519: Method 89

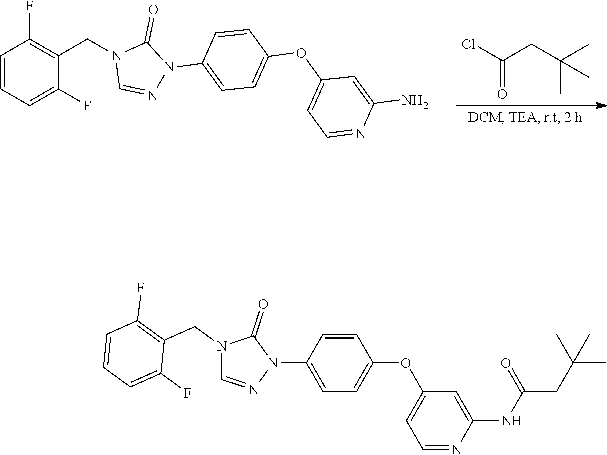

To a solution of 2-[4-[(2-amino-4-pyridyl)oxy]phenyl]-4-[(2,6-difluorophenyl)methyl]-1,2,4-triazol-3-one (1.00 eq, 90 mg, 0.228 mmol) and triethylamine (3.00 eq, 69 mg, 0.683 mmol) in DCM (10 mL), was added 3,3-dimethylbutanoyl chloride (1.00 eq, 31 mg, 0.228 mmol) at room temperature under N₂. After addition, the mixture was stirred at room temperature and stirred for 2 h. Then the mixture was added with water (20 mL) and extracted with DCM (10 mL×3). The organics were then combined and dried (Na₂SO₄) before concentration to dryness. The crude was purified with Prep-HPLC to give the product N-[4-[4-[4-[(2,6-difluorophenyl)methyl]-5-oxo-1,2,4-triazol-1-yl]phenoxy]-2-pyridyl]-3,3-dim ethyl-butanamide (1.1 mg, 0.00212 mmol, 0.93% yield) as a white solid. MS (m/z): 494.2 [M+H]⁺. ¹H NMR (400 MHz, DMSO-d₆) δ 10.53 (s, 1H), 8.34 (s, 1H), 8.19 (d, J=5.9 Hz, 1H), 8.00-7.89 (m, 2H), 7.62 (d, J=2.4 Hz, 1H), 7.52-7.45 (m, 1H), 7.36-7.26 (m, 2H), 7.17 (t, J=8.1 Hz, 2H), 6.71 (dd, J=5.8, 2.4 Hz, 1H), 4.99 (s, 2H), 2.24 (s, 2H), 0.97 (s, 9H).

| Compound No. | | | Appearance | | MS(m/z) |
|---|---|---|---|---|---|
| Method | Activity | Structure and Name | and Yield | ¹H NMR Data | [M + H]⁺ |
| Example 519 Method 89 | +++ | 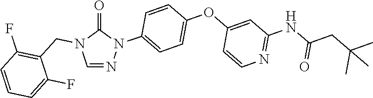 [SMILES: CC(C)(C)CC(=O)Nc1cc(Oc2ccc(-n3ncn(Cc4c(F)cccc4F)c3=O)cc2)ccn1] N-[4-[4-[4-[(2,6-difluorophenyl) methyl]-5-oxo-1,2,4-triazol-1-yl] phenoxy]-2-pyridyl]-3,3- dimethyl-butanamide | white solid, yield: 0.93% | ¹H NMR (400 MHZ, DMSO-d₆) δ 10.53 (s, 1H), 8.34 (s, 1H), 8.19 (d, J = 5.9 Hz, 1H), 8.00-7.89 (m, 2H), 7.62 (d, J = 2.4 Hz, 1H), 7.52-7.45 (m, 1H), 7.36-7.26 (m, 2H), 7.17 (t, J = 8.1 Hz, 2H), 6.71 (dd, J = 5.8, 2.4 Hz, 1H), 4.99 (s, 2H), 2.24 (s, 2H), 0.97 (s, 9H). | 494.2 |

### Example 520: Method 90

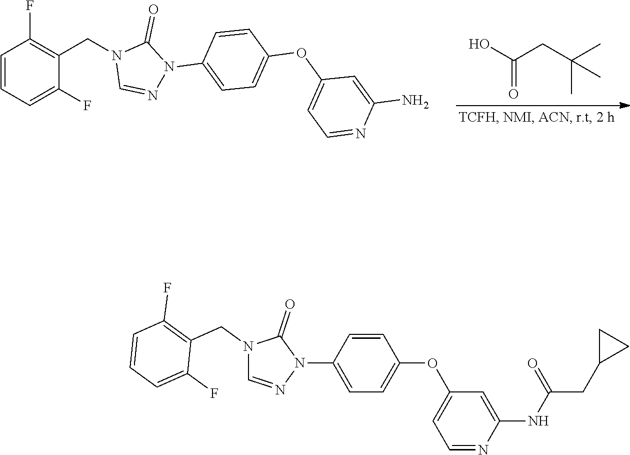

A solution of 2-[4-[(2-amino-4-pyridyl)oxy]phenyl]-4-[(2,6-difluorophenyl)methyl]-1,2,4-triazol-3-one (1.00 eq, 100 mg, 0.253 mmol), 2-cyclopropylacetic acid (1.20 eq, 30 mg, 0.304 mmol), TCFH (1.20 eq, 85 mg, 0.304 mmol) and NMI (3.50 eq, 73 mg, 0.885 mmol) in MeCN (5 mL) was stirred at 25° C. After addition, the mixture was stirred for 2 h. Then the mixture was added with water (20 mL) and extracted with DCM (10 mL×3). The organics were then combined and dried (Na₂SO₄) before concentration to dryness. The crude was purified with Prep-HPLC to give the product 2-cyclopropyl-N-[4-[4-[4-[(2,6-difluorophenyl)methyl]-5-oxo-1,2,4-triazol-1-yl]phenoxy]-2-pyridyl]acetamide (8.0 mg, 0.0159 mmol, 6.29% yield) as white solid. MS (m/z): 478.2 [M+H]⁺. ¹H NMR (400 MHz, DMSO-d₆) δ 10.45 (s, 1H), 8.20 (s, 1H), 8.06 (d, J=5.9 Hz, 1H), 7.88-7.76 (m, 2H), 7.47-7.25 (m, 2H), 7.21-7.07 (m, 2H), 7.02 (t, J=8.1 Hz, 2H), 6.62 (dd, J=5.9, 2.4 Hz, 1H), 4.85 (s, 2H), 2.09 (d, J=7.1 Hz, 2H), 0.84 (m, 1H), 0.38-0.21 (m, 2H), 0.07-0.09 (m, 2H).

| Compound No. | | | Appearance | | MS(m/z) |
|---|---|---|---|---|---|
| Method | Activity | Structure and Name | and Yield | ¹H NMR Data | [M + H]⁺ |
| Example 520 Method 90 | +++ | 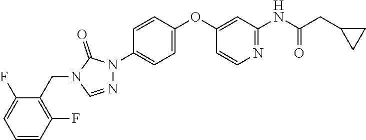 [SMILES: O=C(CC1CC1)Nc1cc(Oc2ccc(-n3ncn(Cc4c(F)cccc4F)c3=O)cc2)ccn1] 2-cyclopropyl-N-[4-[4-[4-[(2,6- difluorophenyl)methyl]-5-oxo- 1,2,4-triazol-1-yl]phenoxy]-2- pyridyl]acetamide | white solid, yield: 6.29% | ¹H NMR (400 MHZ, DMSO-d₆) δ 10.45 (s, 1H), 8.20 (s, 1H), 8.06 (d, J = 5.9 Hz, 1H), 7.88-7.76 (m, 2H), 7.47-7.25 (m, 2H), 7.21-7.07 (m, 2H), 7.02 (t, J = 8.1 Hz, 2H), 6.62 (dd, J = 5.9, 2.4 Hz, 1H), 4.85 (s, 2H), 2.09 (d, J = 7.1 Hz, 2H), 0.84 (m, 1H), 0.38-0.21 (m, 2H), 0.07-0.09 (m, 2H). | 478.2 |

### Example 521: Method 91

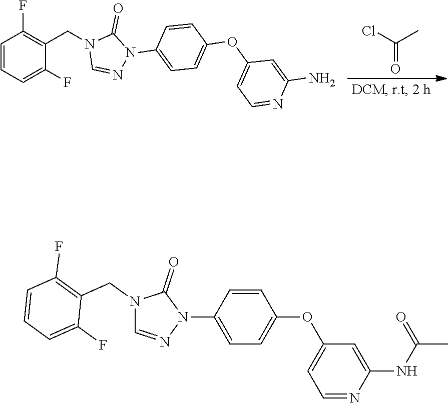

To a solution of 2-[4-[(2-amino-4-pyridyl)oxy]phenyl]-4-[(2,6-difluorophenyl)methyl]-1,2,4-triazol-3-one (1.00 eq, 50 mg, 0.126 mmol) in DCM (5 mL), was added acetyl chloride (1.00 eq, 9.9 mg, 0.126 mmol) at 25° C. under N₂. After addition, the mixture was stirred at room temperature and stirred for 2 h. The reaction mixture was concentrated to dryness and purified by prep-TLC (20% MeOH in DCM) to give the product N-[4-[4-[4-[(2,6-difluorophenyl)methyl]-5-oxo-1,2,4-triazol-1-yl]phenoxy]-2-pyridyl]acetamide (2.9 mg,0.00630 mmol, 4.98% yield) as a yellow solid. MS (m/z): 438.1 [M+H]⁺. ¹H NMR (400 MHz, DMSO-d₆) δ 10.54 (s, 1H), 8.33 (s, 1H), 8.18 (d, J=5.8 Hz, 1H), 8.01-7.92 (m, 2H), 7.66 (d, J=2.3 Hz, 1H), 7.52-7.45 (m, 1H), 7.29-7.23 (m, 2H), 7.17 (t, J=8.1 Hz, 2H), 6.66 (dd, J=5.7, 2.4 Hz, 1H), 4.99 (s, 2H), 2.03 (s, 3H).

| Compound No. | | | Appearance | | MS(m/z) |
|---|---|---|---|---|---|
| Method | Activity | Structure and Name | and Yield | ¹H NMR Data | [M + H]⁺ |
| Example 521 Method 91 | +++ | 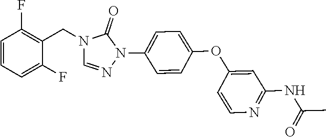 [SMILES: CC(=O)Nc1cc(Oc2ccc(-n3ncn(Cc4c(F)cccc4F)c3=O)cc2)ccn1] N-[4-[4-[4-[(2,6-difluorophenyl) methyl]-5-oxo-1,2,4-triazol-1-yl] phenoxy]-2-pyridyl]acetamide | yellow solid, yield: 4.98% | ¹H NMR (400 MHZ, DMSO-d₆) δ 10.54 (s, 1H), 8.33 (s, 1H), 8.18 (d, J = 5.8 Hz, 1H), 8.01-7.92 (m, 2H), 7.66 (d, J = 2.3 Hz, 1H), 7.52-7.45 (m, 1H), 7.29-7.23 (m, 2H), 7.17 (t, J = 8.1 Hz, 2H), 6.66 (dd, J = 5.7, 2.4 Hz, 1H), 4.99 (s, 2H), 2.03 (s, 3H). | 438.1 |

### Example 522: Method 92

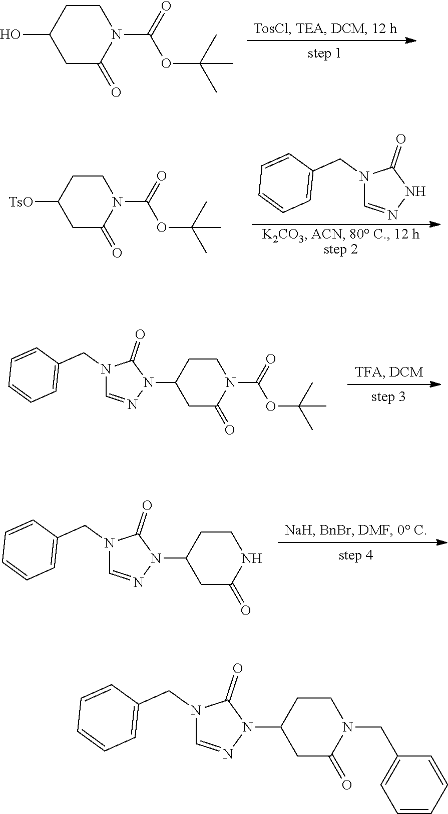

Step 1: To a solution of tert-butyl 4-hydroxy-2-oxopiperidine-1-carboxylate (1.00 eq, 1 g, 4.65 mmol), and TEA (2.0 eq, 1.33 g, 6.97 mmol) in DCM (15 mL), was added TsCl (1.5 eq, 1.33 g, 6.97 mmol) at 25° C. under N₂. After addition, the mixture was stirred at room temperature for 12 h. The reaction mixture was washed with NaHCO₃ aq. (10 mL) and brine (10 mL) and concentrated to give the product (2 g, crude) as a yellow solid, which was used directly for next step. MS (m/z): 370.1 [M+H]⁺.

### Step 2: tert-butyl 4-(4-benzyl-5-oxo-4,5-dihydro-1H-1,2,4-triazol-1-yl)-2-oxopiperidine-1-carboxylate

To a solution of 4-(2,6-difluorobenzyl)-2,4-dihydro-3H-1,2,4-triazol-3-one (1.00 eq, 200 mg, 1.19 mmol) and tert-butyl 2-oxo-4-(tosyloxy)piperidine-1-carboxylate (2.00 eq, 700 mg, 2.39 mmol) in ACN (20 mL), was added K₂CO₃ (2.00 eq, 240 mg, 2.39 mmol) at 25° C. After addition, the mixture was stirred at 80° C. for 15 h. The reaction mixture was cooled and concentrated. The residue was purified by silica gel column (DCM/MeOH=20/1) to give the product (250 mg, 58.8% yield) as an off-white solid. MS (m/z): 373.2 [M+H]⁺. ¹H NMR (400 MHz, DMSO-d₆) δ 8.10 (d, J=3.1 Hz, 1H), 7.49-7.16 (m, 5H), 4.79 (d, J=3.1 Hz, 2H), 4.62-4.54 (m, 1H), 3.61 (dd, J=16.8, 6.5 Hz, 2H), 3.04-2.79 (m, 1H), 2.68-2.59 (m, 1H), 2.13-1.93 (m, 2H), 1.61-1.27 (m, 9H).

### Step 3: 4-(4-benzyl-5-oxo-4,5-dihydro-1H-1,2,4-triazol-1-yl)piperidin-2-one

4-(4-benzyl-5-oxo-4,5-dihydro-1H-1,2,4-triazol-1-yl)-2-oxopiperidine-1-carboxylate (250 mg, 0.671 mmoL) was dissolved in 4 mL DCM. 3 mL DCM/TFA (V/V=1/1) was added to the solution. The reaction mixture was stirred at r.t. for 1h. The solvent was evaporated to dryness to give the product of TFA salt (195 mg, 98% yield) as a yellow gum and used for next step without further purification. MS (m/z): 272.1 [M+H]⁺. ¹H NMR (400 MHz, DMSO-d₆) δ 8.09 (s, 1H), 7.60 (s, 1H), 7.42-7.26 (m, 5H), 4.79 (s, 2H), 4.52-4.42 (m, 1H), 4.00-3.91 (m, 2H), 3.22-3.11 (m, 2H), 1.91 (d, J=6.7 Hz, 2H).

### Step 4: 1-benzyl-4-(4-benzyl-5-oxo-4,5-dihydro-1H-1,2,4-triazol-1-yl)piperidin-2-one

4-(4-benzyl-5-oxo-4,5-dihydro-1H-1,2,4-triazol-1-yl)piperidin-2-one (100 mg, 0.37 mmol) was dissolved in 2 mL DMF. NaH (29 mg, 0.73 mmol) was added to the solution at 0° C. The reaction mixture was stirred at 0° C. for 1h. BnBr (126 mg, 0.73 mmol) was added. The reaction mixture was stirred at 0° C. for 2 h. The reaction was quenched with a NH₄Cl solution and extracted with EA (3×5 mL). The organic layers were combined and purified by prep-HPLC to give the desired product (18 mg, 13.5% yield) as a white solid. MS (m/z): 363.2 [M+H]⁺. ¹H NMR (400 MHz, DMSO-d₆) δ 8.10 (d, J=2.4 Hz, 1H), 7.40-7.22 (m, 10H), 4.79 (s, 2H), 4.62 (d, J=14.9 Hz, 1H), 4.56-4.28 (m, 1H), 4.44 (d, J=15.0 Hz, 1H), 3.24-3.12 (m, 2H), 2.67 (m, 2H), 2.07-1.95 (m, 2H).

| Compound No. | | | Appearance | | MS(m/z) |
|---|---|---|---|---|---|
| Method | Activity | Structure and Name | and Yield | ¹H NMR Data | [M + H]⁺ |
| Example 522 Method 92 | + | 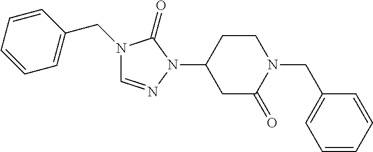 [SMILES: O=C1CC(n2ncn(Cc3ccccc3)c2=O)CCN1Cc1ccccc1] | yellow gum, yield: 98% | ¹H NMR (400 MHZ, DMSO-d₆) δ 8.10 (d, J = 2.4 Hz, 1H), 7.40-7.22 (m, 10H), 4.79 (s, 2H), 4.62 (d, J = 14.9 Hz, 1H), 4.56-4.28 (m, 1H), 4.44 (d, J = 15.0 Hz, 1H), 3.24-3.12 (m, 2H), 2.67 (m, 2H), 2.07-1.95 (m, 2H). | 363.2 |
| | | 1-benzyl-4-(4-benzyl-5-oxo-4,5- | | | |
| | | dihydro-1H-1,2,4-triazol-1-yl) | | | |
| | | piperidin-2-one | | | |

### Example 523: Method 93

### 2-(4-((2-(cyclopropylamino)pyridin-4-yl)oxy)phenyl)-4-(2,6-difluorobenzyl)-2,4-dihydro-3H-1,2,4-triazol-3-one

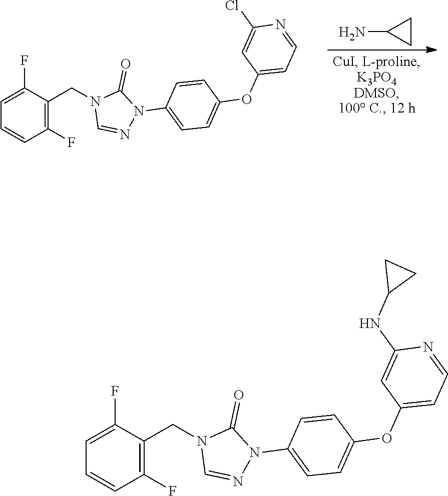

To a solution of 2-(4-((2-chloropyridin-4-yl)oxy)phenyl)-4-(2,6-difluorobenzyl)-2,4-dihydro-3H-1,2,4-triazol-3-one (100 mg, 241.1 umol) in DMSO (4 mL) were added cyclopropanamine (28 mg, 482.2 umol), L-proline (55 mg, 482.2 umol), CuI (46 mg, 241.1 umol) and K₃PO₄ (205 mg, 964.3 umol) under Ar. The reaction mixture was stirred at 100° C. for 12 h. The target product was detected by LCMS. The reaction mixture was taken up in EtOAc (100 mL) and the organics were washed with 2×50 mL water and then 1×50 mL saturated brine solution. The organics were then separated and dried (MgSO4) before concentration to dryness. The crude was then purified by HPLC. 2-(4-((2-(cyclopropylamino)pyridin-4-yl)oxy)phenyl)-4-(2,6-difluorobenzyl)-2,4-dihydro-3H-1,2,4-triazol-3-one (14 mg, 13.3% yield) was obtained as a white solid. MS (m/z): 436.1 [M+H]⁺. ¹H NMR (400 MHz, CDCl₃) δ 8.13 (d, J=8.6 Hz, 2H), 7.73 (brs, 1H), 7.64 (s, 1H), 7.45-7.33 (m, 1H), 7.27 (s, 1H), 7.19 (d, J=8.6 Hz, 2H), 7.00 (t, J=7.8 Hz, 2H), 6.49-6.30 (m, 2H), 5.00 (s, 2H), 2.38 (s, 1H), 0.77 (d, J=6.4 Hz, 2H), 0.65 (s, 2H).

The synthesis procedure of Examples (Compounds) 524-533 was similar to the procedure of Example 523.

| Compound No. | | | Appearance | | MS(m/z) |
|---|---|---|---|---|---|
| Method | Activity | Structure and Name | and Yield | ¹H NMR Data | [M + H]⁺ |
| Example 523 Method 93 | +++ | 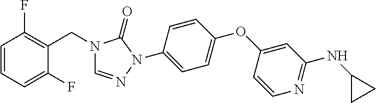 [SMILES: O=c1n(Cc2c(F)cccc2F)cnn1-c1ccc(Oc2ccnc(NC3CC3)c2)cc1] | white solid, yield: 13.3% | ¹H NMR (400 MHZ, CDCl₃) δ 8.13 (d, J = 8.6 Hz, 2H), 7.73 (brs, 1H), 7.64 (s, 1H), 7.45-7.33 (m, 1H), 7.27 (s, 1H), 7.19 (d, J = 8.6 Hz, 2H), 7.00 (t, J = 7.8 Hz, 2H), 6.49-6.30 (m, 2H), 5.00 (s, 2H), 2.38 (s, 1H), 0.77 (d, J = 6.4 Hz, | 436.1 |
| | | | | 2H), 0.65 (s, 2H). | |
| | | 2-(4-((2-(cyclopropylamino) | | | |
| | | pyridin-4-yl)oxy)phenyl)-4-(2,6- | | | |
| | | difluorobenzyl)-2,4-dihydro-3H- | | | |
| | | 1,2,4-triazol-3-one | | | |
| Example 524 Method 93 | ++++ | 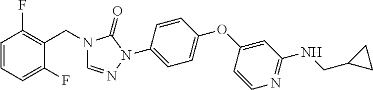 [SMILES: O=c1n(Cc2c(F)cccc2F)cnn1-c1ccc(Oc2ccnc(NCC3CC3)c2)cc1] | Off-white solid, yield: 10.2% | ¹H NMR (400 MHZ, CDCl₃) δ 8.19-8.04 (m, 2H), 7.74-7.57 (m, 2H), 7.39 (ddd, J = 15.0, 8.5, 6.5 Hz, 1H), 7.22-7.09 (m, 2H), 7.00 (t, J = 7.9 Hz, 2H), 6.36 (dd, J = 7.0, 2.3 Hz, 1H), 5.93 (d, | 450.2 |
| | | | | J = 2.3 Hz, 1H), 5.01 (s, 2H), | |
| | | 2-(4-((2-((cyclopropylmethyl) | | 3.56-3.48 (m, 2H), 2.88-2.82 (m, | |
| | | amino)pyridin-4-yl)oxy)phenyl)-4- | | 1H), 0.58 (dd, J = 7.9, 5.2 Hz, 2H), | |
| | | (2,6-difluorobenzyl)-2,4-dihydro- | | 0.22 (q, J = 5.3 Hz, 2H). | |
| | | 3H-1,2,4-triazol-3-one | | | |
| Example 525 Method 93 | +++ | 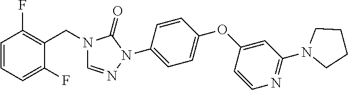 [SMILES: O=c1n(Cc2c(F)cccc2F)cnn1-c1ccc(Oc2ccnc(N3CCCC3)c2)cc1] | white solid, yield: 12% | NA | 450.2 |
| | | 4-(2,6-difluorobenzyl)-2-(4-((2- | | | |
| | | (pyrrolidin-1-yl)pyridin-4-yl)oxy) | | | |
| | | phenyl)-2,4-dihydro-3H-1,2,4- | | | |
| | | triazol-3-one | | | |
| Example 526 Method 93 | ++ | 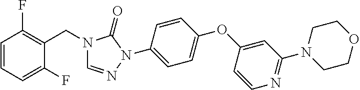 [SMILES: O=c1n(Cc2c(F)cccc2F)cnn1-c1ccc(Oc2ccnc(N3CCOCC3)c2)cc1] | white solid, yield: 13.4% | ¹H NMR (400 MHZ, CDCl₃) δ 8.13 (dd, J = 13.7, 8.1 Hz, 3H), 7.63 (d, J = 11.3 Hz, 1H), 7.39 (tt, J = 8.4, 6.5 Hz, 1H), 7.19- 7.11 (m, 2H), 7.00 (t, J = 7.9 Hz, 2H), 6.53 (dd, J = 7.0, | 466.2 |
| | | | | 2.2 Hz, 1H), 6.13 (d, J = 2.2 Hz, | |
| | | 4-(2,6-difluorobenzyl)-2-(4-((2- | | 1H), 5.00 (s, 2H), 3.81 (t, J = 4.8 | |
| | | morpholinopyridin-4-yl)oxy)phenyl)- | | Hz, 4H), 3.55 (t, J = 4.9 Hz, 4H). | |
| | | 2,4-dihydro-3H-1,2,4-triazol-3-one | | | |
| Example 527 Method 93 | +++ | 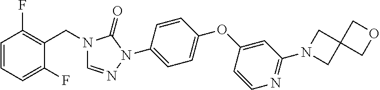 [SMILES: O=c1n(Cc2c(F)cccc2F)cnn1-c1ccc(Oc2ccnc(N3CC4(COC4)C3)c2)cc1] | white solid, yield: 13.4% | ¹H NMR (400 MHZ, CDCl₃) δ 7.98 (d, J = 8.6 Hz, 2H), 7.79- 7.60 (m, 1H), 7.56 (s, 1H), 7.40-7.23 (m, 1H), 7.05 (d, J = 8.3 Hz, 2H), 6.90 (t, J = 7.9 Hz, 2H), 6.33 (d, J = 6.3 | 478.2 |
| | | | | Hz, 1H), 5.57 (s, 1H), 4.91 | |
| | | 2-(4-((2-(2-oxa-6-azaspiro[3.3] | | (s, 2H), 4.12-3.44 (m, 8H). | |
| | | heptan-6-yl)pyridin-4-yl)oxy)pheny)- | | | |
| | | 4-(2,6-difluorobenzyl)-2,4-dihydro- | | | |
| | | 3H-1,2,4-triazol-3-one | | | |
| Example 528 Method 93 | +++ | 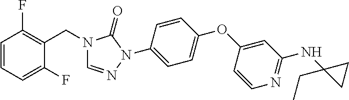 [SMILES: CCC1(Nc2cc(Oc3ccc(-n4ncn(Cc5c(F)cccc5F)c4=O)cc3)ccn2)CC1] | white solid, yield: 2.7% | NA | 464.2 |
| | | 4-(2,6-difluorobenzyl)-2-(4-((2-((1- | | | |
| | | ethylcyclopropyl)amino)pyridin- | | | |
| | | 4-yl)oxy)phenyl)-2,4-dihydro-3H- | | | |
| | | 1,2,4-triazol-3-one | | | |
| Example 529 Method 93 | +++ | 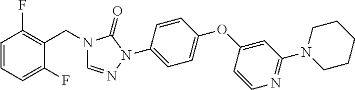 [SMILES: O=c1n(Cc2c(F)cccc2F)cnn1-c1ccc(Oc2ccnc(N3CCCCC3)c2)cc1] | white solid, yield: 2.7% | ¹H NMR (400 MHZ, CDCl₃) δ 8.21-8.01 (m, 3H), 7.64 (s, 1H), 7.39 (tt, J = 8.4, 6.5 Hz, 1H), 7.20-7.09 (m, 2H), 7.08-6.93 (m, 2H), 6.42 (dd, J = 7.1, 2.2 Hz, 1H), 6.11 (d, J = 2.3 Hz, 1H), 5.00 (s, | 464.2 |
| | | | | 2H), 3.64-3.48 (m, 4H), 1.80-1.62 | |
| | | 4-(2,6-difluorobenzyl)-2-(4-((2- | | (m, 6H). | |
| | | (piperidin-1-yl)pyridin-4-yl)oxy) | | | |
| | | phenyl)-2,4-dihydro-3H-1,2,4-triazol- | | | |
| | | 3-one | | | |
| Example 530 Method 93 | +++ | 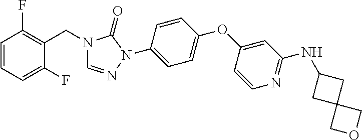 [SMILES: O=c1n(Cc2c(F)cccc2F)cnn1-c1ccc(Oc2ccnc(NC3CC4(COC4)C3)c2)cc1] | white solid, yield: 5.1% | NA | 492.2 |
| | | 2-(4-((2-((2-oxaspiro[3.3]heptan- | | | |
| | | 6-yl)amino)pyridin-4-yl)oxy)phenyl)- | | | |
| | | 4-(2,6-difluorobenzyl)-2,4-dihydro- | | | |
| | | 3H-1,2,4-triazol-3-one | | | |
| Example 531 Method 93 | + | 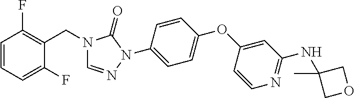 [SMILES: CC1(Nc2cc(Oc3ccc(-n4ncn(Cc5c(F)cccc5F)c4=O)cc3)ccn2)COC1] | white solid, yield: 3.6% | NA | 466.2 |
| | | 4-(2,6-difluorobenzyl)-2-(4-((2-((3- | | | |
| | | methyloxetan-3-yl)amino)pyridin- | | | |
| | | 4-yl)oxy)phenyl)-2,4-dihydro- | | | |
| | | 3H-1,2,4-triazol-3-one | | | |
| Example 532 Method 93 | ++++ | 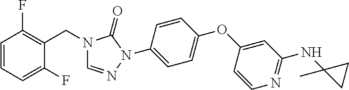 [SMILES: CC1(Nc2cc(Oc3ccc(-n4ncn(Cc5c(F)cccc5F)c4=O)cc3)ccn2)CC1] | white solid, yield: 2.8% | NA | 450.2 |
| | | 4-(2,6-difluorobenzyl)-2-(4-((2-((1- | | | |
| | | methylcyclopropyl)amino)pyridin- | | | |
| | | 4-yl)oxy)phenyl)-2,4-dihydro- | | | |
| | | 3H-1,2,4-triazol-3-one | | | |
| Example 533 Method 93 | ++++ | 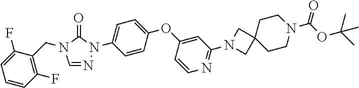 [SMILES: CC(C)(C)OC(=O)N1CCC2(CC1)CN(c1cc(Oc3ccc(-n4ncn(Cc5c(F)cccc5F)c4=O)cc3)ccn1)C2] | white solid, yield: 15.1% | ¹H NMR (400 MHZ, DMSO-d₆) δ 8.36 (s, 1H), 7.99 (t, J = 7.6 Hz, 3H), 7.66-7.51 (m, 3H), 7.36-7.27 (m, 2H), 7.17 (t, J = 8.1 Hz, 2H), 5.00 (s, 2H), 3.92-3.66 (m, 8H), 1.72-1.62 (m, 4H), 1.38 (s, 9H). | 605.2 |
| | | tert-butyl2-(4-(4-(4-(2,6-difluorobenzyl)- | | | |
| | | 5-oxo-4,5-dihydro-1H-1,2, | | | |
| | | 4-triazol-1-yl)phenoxy)pyridin-2- | | | |
| | | yl)-2,7-diazaspiro[3.5]nonane-7- | | | |
| | | carboxylate | | | |

### Example 534: Method 94

### Ethyl 4-(4-(4-(2,6-difluorobenzyl)-5-oxo-4,5-dihydro-1H-1,2,4-triazol-1-yl)-2-fluorophenoxy)oxazole-5-carboxylate

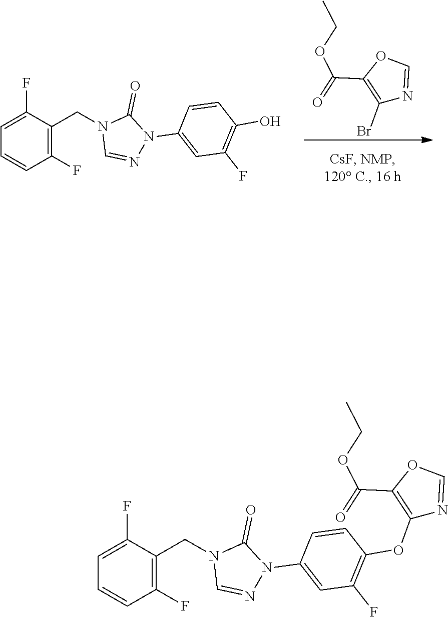 [SMILES: O=c1n(Cc2c(F)cccc2F)cnn1-c1ccc(O)c(F)c1 ; CCOC(=O)c1ocnc1Br ; CCOC(=O)c1ocnc1Oc1ccc(-n2ncn(Cc3c(F)cccc3F)c2=O)cc1F]

A solution of ethyl 4-bromooxazole-5-carboxylate (20 mg, 0.091 mmol), 4-(2,6-difluorobenzyl)-2-(3-fluoro-4-hydroxyphenyl)-2,4-dihydro-3H-1,2,4-triazol-3-one (29.2 mg, 0.091 mmol) in NMP (3 mL) was added CsF (16.6 mg, 0.11 mmol). The reaction mixture was stirred at 120° C. for 16 h. The mixture was filtered and purified by prep-HPLC with the following conditions: column: Sunfire 5 um 19-150 nm; Mobile phase B: ACN; Flow rate: 20 mL/min; Gradient: 40% B-65% B in 8 min; Detector: 220 nm. The fractions containing the desired product were collected at 55% B and concentrated under reduced pressure to afford the titled product (4 mg, 15.6% yield) as a yellow oil. MS (m/z): 461.0 [M+H]⁺. ¹H NMR (400 MHz, CDCl₃) δ 7.95-7.98 (m, 1H), 7.85 (t, J=7.6 Hz, 1H), 7.73 (s, 1H), 7.61 (s, 1H), 7.29-7.42 (m, 2H), 7.01 (t, J=7.6 Hz, 2H), 5.00 (s, 2H), 4.41-4.47 (m, 2H), 1.41 (t, J=7.2 Hz, 3H).

| Compound No. | | | Appearance | | MS(m/z) |
|---|---|---|---|---|---|
| Method | Activity | Structure and Name | and Yield | ¹H NMR Data | [M + H]⁺ |
| Example 534 Method 94 | ++++ | 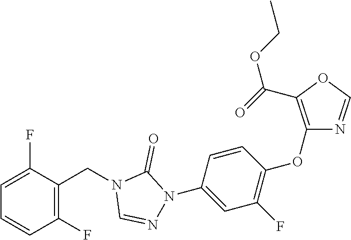 [SMILES: CCOC(=O)c1ocnc1Oc1ccc(-n2ncn(Cc3c(F)cccc3F)c2=O)cc1F] | yellow oil, yield: 15.6% | ¹H NMR (400 MHZ, CDCl₃) δ7.95-7.98 (m, 1H), 7.85 (t, J = 7.6 Hz, 1H), 7.73 (s, 1H), 7.61 (s, 1H), 7.29-7.42 (m, 2H), 7.01 (t, J = 7.6 Hz, 2H), 5.00 (s, 2H), 4.41-4.47 (m, 2H), 1.41 (t, J = 7.2 Hz, 3H). | 461.0 |
| | | ethyl | | | |
| | | 4-(4-(4-(2,6-difluorobenzyl)-5-oxo- | | | |
| | | 4,5-dihydro-1H-1,2,4-triazol-1- | | | |
| | | yl)-2-fluorophenoxy)oxazole-5- | | | |
| | | carboxylate | | | |

### Example 535: Method 95

### 4-(2,6-difluorobenzyl)-2-(4-((2-(3-hydroxy-3-methylbut-1-yn-1-yl)pyridin-4-yl)oxy)phenyl)-2,4-dihydro-3H-1,2,4-triazol-3-one

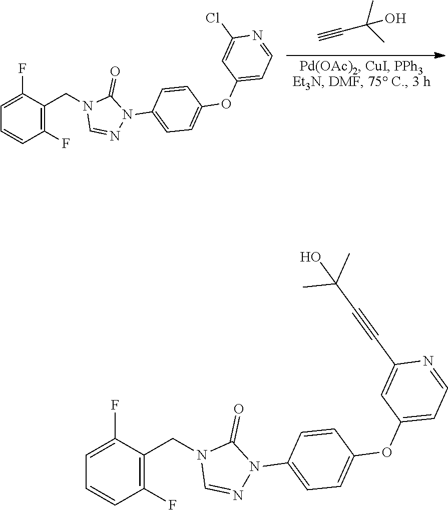

To a solution of 2-(4-((2-chloropyridin-4-yl)oxy)phenyl)-4-(2,6-difluorobenzyl)-2,4-dihydro-3H-1,2,4-triazol-3-one (100 mg, 241.1 umol) in DMF (5 mL) were added 2-methylbut-3-yn-2-ol (40 mg, 482.2 umol), Pd(OAc)₂ (54 mg, 241.1 umol), CuI (46 mg, 241.1 umol) and Et₃N (98 mg, 241.1 umol) under Ar. The reaction mixture was stirred at 75° C. for 3 h. The target product was detected by LCMS. The reaction mixture was taken up in EtOAc (100 mL) and the organics were washed with 2×50 mL water and then 1×50 mL saturated brine solution. The organics were then separated and dried (MgSO₄) before concentration to dryness. The crude was then purified by reversed-phase column. 4-(2,6-difluorobenzyl)-2-(4-((2-(3-hydroxy-3-methylbut-1-yn-1-yl)pyridin-4-yl)oxy)phenyl)-2,4-dihydro-3H-1,2,4-triazol-3-one (16 mg, 14.4% yield) was obtained as a white solid. MS (m/z): 463.1 [M+H]⁺. ¹H NMR (400 MHz, DMSO-d₆) δ 8.44 (s, 1H), 8.34 (s, 1H), 8.04-7.91 (m, 2H), 7.49 (tt, J=8.5, 6.6 Hz, 1H), 7.37-7.27 (m, 2H), 7.24-7.12 (m, 2H), 6.99 (dd, J=5.7, 2.6 Hz, 1H), 6.88 (d, J=2.5 Hz, 1H), 4.99 (s, 2H), 1.43 (s, 6H).

| Compound | | | | | |
|---|---|---|---|---|---|
| No. | | | Appearance | | MS(m/z) |
| Method | Activity | Structure and Name | and Yield | ¹H NMR Data | [M + H]⁺ |
| Example 535 Method 95 | +++ | 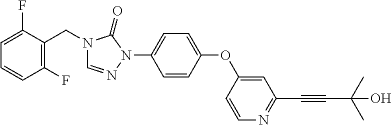 [SMILES: CC(C)(O)C#Cc1cc(Oc2ccc(-n3ncn(Cc4c(F)cccc4F)c3=O)cc2)ccn1] | white solid, yield: 14.4% | ¹H NMR (400 MHZ, DMSO-d₆) δ 8.44 (s, 1H), 8.34 (s, 1H), 8.04-7.91 (m, 2H), 7.49 (tt, J = 8.5, 6.6 Hz, 1H), 7.37-7.27 (m, 2H), 7.24-7.12 (m, 2H), 6.99 (dd, J = 5.7, 2.6 Hz, 1H), 6.88 (d, J = 2.5 Hz, 1H), 4.99 (s, 2H), 1.43 (s, | 463.1 |
| | | | | 6H). | |
| | | 4-(2,6-difluorobenzyl)-2-(4-((2- | | | |
| | | (3-hydroxy-3-methylbut-1-yn-1- | | | |
| | | yl)pyridin-4-yl)oxy)phenyl)-2,4- | | | |
| | | dihydro-3H-1,2,4-triazol-3-one | | | |

### Example 536: Method 96

### 4-(2,6-difluorobenzyl)-2-(4-(3,6-dihydro-2H-pyran-4-yl)phenyl)-2,4-dihydro-3H-1,2,4-triazol-3-one

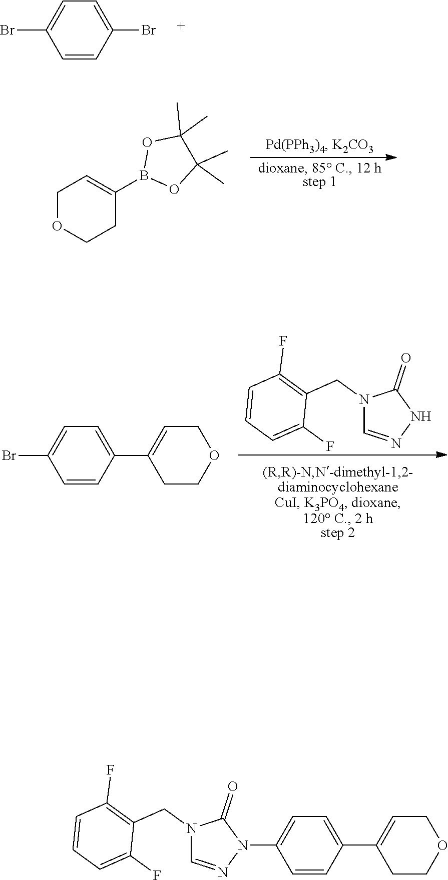

### Step 1: 4-(4-bromophenyl)-3,6-dihydro-2H-pyran

To a solution of 1,4-dibromobenzene (100 mg, 0.424 mmol) in 1,4-Dioxane (8 mL) and Water (2 mL) were added 3,6-Dihydro-4-(4,4,5,5-tetramethyl-1,3,2-dioxaborolan-2-yl)-2H-pyran (89 mg, 0.424 mmol), tetrakis(triphenylphosphine)palladium (49 mg, 0.0424 mmol) and tetrakis(triphenylphosphine)palladium (49 mg, 0.0424 mmol) under Ar. The reaction mixture was stirred at 85° C. for 12 h. The reaction was concentrated to dryness and the residue was taken up in EtOAc (100 mL) and the organics were washed with 2×50 mL water and then 1×50 mL saturated brine solution. The organics were then separated and dried (MgSO₄) before concentration to dryness. The crude was then purified by flash column chromatography eluted with 10˜50% EtOAc in PE. The desired fractions were concentrated to dryness in vacuo. 4-(4-bromophenyl)-3,6-dihydro-2H-pyran (86 mg, 84.8% yield) was obtained as a white solid. MS (m/z): 239.1 [M+H]⁺.

### Step 2: 4-(2,6-difluorobenzyl)-2-(4-(3,6-dihydro-2H-pyran-4-yl)phenyl)-2,4-dihydro-3H-1,2,4-triazol-3-one

To a solution of 4-(4-bromophenyl)-3,6-dihydro-2H-pyran (1.00 eq, 113 mg, 0.474 mmol) in 1,4-Dioxane (6 mL) were added 4-[(2,6-difluorophenyl)methyl]-1H-1,2,4-triazol-5-one (100 mg, 0.474 mmol), CuI (90 mg, 0.474 mmol), (R,R)—N,N′-dimethyl-1,2-diaminocyclohexane (67 mg, 0.474 mmol) and K₃PO₄ (302 mg, 1.42 mmol) under Ar. The reaction mixture was stirred at 120° C. for 2 h. The target product was detected by LCMS. The reaction mixture was concentrated to dryness and the residue was taken up in EtOAc (100 mL) and the organics were washed with 2×50 mL water and then 1×50 mL saturated brine solution. The organics were then separated and dried (MgSO₄) before concentration to dryness. The crude was then purified by HPLC. 4-[(2,6-difluorophenyl)methyl]-2-[4-(3,6-dihydro-2H-pyran-4-yl)phenyl]-1,2,4-triazol-3-one (3.0 mg, 1.72% yield) was obtained as a white solid. MS (m/z): 370.1 [M+H]⁺. ¹H NMR (400 MHz, DMSO-d₆) δ 7.89-.81 (m, 1H), 7.58-7.39 (m, 3H), 7.28-7.03 (m, 4H), 6.32-6.20 (m, 1H), 4.98 (s, 2H), 4.22 (d, J=2.9 Hz, 2H), 3.82 (t, J=5.4 Hz, 2H), 1.99 (dt, J=13.5, 7.2 Hz, 2H).

| Compound No. | | | Appearance | | MS(m/z) |
|---|---|---|---|---|---|
| Method | Activity | Structure and Name | and Yield | ¹H NMR Data | [M + H]⁺ |
| Example 536 Method 96 | ++ | 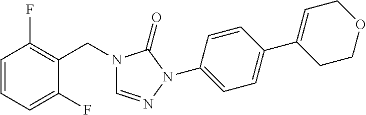 [SMILES: O=c1n(Cc2c(F)cccc2F)cnn1-c1ccc(C2=CCOCC2)cc1] | white solid, yield: 1.7% | ¹H NMR (400 MHZ, DMSO-d₆) δ 7.89-.81 (m, 1H), 7.58-7.39 (m, 3H), 7.28-7.03 (m, 4H), 6.32-6.20 (m, 1H), 4.98 (s, 2H), 4.22 (d, J = 2.9 Hz, 2H), 3.82 (t, J = 5.4 Hz, 2H), 1.99 (dt, J = 13.5, 7.2 Hz, 2H). | 370.1 |
| | | 4-(2,6-difluorobenzyl)-2-(4-(3,6- | | | |
| | | dihydro-2H-pyran-4-yl)phenyl)- | | | |
| | | 2,4-dihydro-3H-1,2,4-triazol-3-one | | | |

### Example 537: Method 97

### 2-(4-((3-(3-amino-3-methylazetidin-1-yl)-5-methyl-1H-pyrazol-1-yl)methyl)phenyl)-4-(2,6-difluorobenzyl)-2,4-dihydro-3H-1,2,4-triazol-3-one

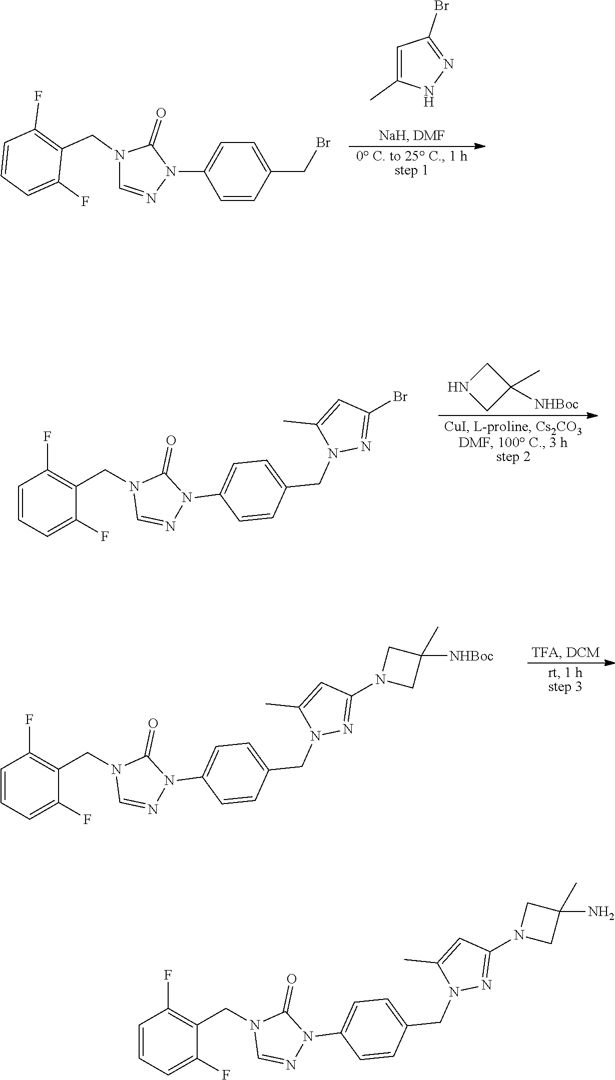

### Step 1: 2-(4-((3-bromo-5-methyl-1H-pyrazol-1-yl)methyl)phenyl)-4-(2,6-difluorobenzyl)-2,4-dihydro-3H-1,2,4-triazol-3-one

To a solution of 3-bromo-5-methyl-1H-pyrazole (254 mg, 1.58 mmol) in DMF (15 mL) was added NaH (105 mg, 2.63 mmol) under Ar at 0° C. The reaction mixture was stirred at 0° C. for 30 min and then 2-[4-(bromomethyl)phenyl]-4-[(2,6-difluorophenyl) methyl]-1,2,4-triazol-3-one (500 mg, 1.32 mmol) was added. The reaction mixture was stirred at 25° C. for 1 h. The target product was detected by LCMS. The reaction mixture was taken up in EtOAc (200 mL) and the organics were washed with 2×100 mL water and then 1×100 mL saturated brine solution. The organics were then separated and dried (MgSO₄) before concentration to dryness. The crude was then purified by flash column chromatography eluted with 10-60% EtOAc in PE. The desired fractions were concentrated to dryness under vacuum. 2-[4-[(3-bromo-5-methyl-pyrazol-1-yl)methyl]phenyl]-4-[(2,6-difluorophenyl)methyl]-1,2,4-triazol-3-one (310 mg, 51.21% yield) was obtained as a white solid. MS (m/z): 460.1 [M+H]⁺.

### Step 2: tert-butyl (1-(1-(4-(4-(2,6-difluorobenzyl)-5-oxo-4,5-dihydro-1H-1,2,4-triazol-1-yl)benzyl)-5-methyl-1H-pyrazol-3-yl)-3-methylazetidin-3-yl)carbamate

To a solution of 2-[4-[(3-bromo-5-methyl-pyrazol-1-yl)methyl]phenyl]-4-[(2,6-difluorophenyl)methyl]-1,2,4-triazol-3-one (100 mg, 0.217 mmol) in DMF (5 mL) were added tert-butyl N-(3-methylazetidin-3-yl)carbamate (61 mg, 0.326 mmol), CuI (41 mg, 0.217 mmol), L-proline (30 mg, 0.261 mmol) and Cs₂CO₃ (212 mg, 0.652 mmol) under Ar. The reaction mixture was stirred at 100° C. for 3 h. The target product was detected by LCMS. The reaction mixture was taken up in EtOAc (00 mL) and the organics were washed with 2×50 mL water and then 1×50 mL saturated brine solution. The organics were then separated and dried (MgSO₄) before concentration to dryness. The crude was then purified by HPLC to give tert-butyl N-[1-[1-[[4-[4-[(2,6-difluorophenyl)methyl]-5-oxo-1,2,4-triazol-1-yl]phenyl]methyl]-5-methyl-pyrazol-3-yl]-3-methyl-azetidin-3-yl]carbamate (10 mg, 8.14% yield) was obtained as a off-white solid. MS (m/z): 566.2 [M+H]⁺.

### Step 3: 2-(4-((3-(3-amino-3-methylazetidin-1-yl)-5-methyl-1H-pyrazol-1-yl)methyl)phenyl)-4-(2,6-difluorobenzyl)-2,4-dihydro-3H-1,2,4-triazol-3-one

To a solution of tert-butyl N-[1-[1-[[4-[4-[(2,6-difluorophenyl)methyl]-5-oxo-1,2,4-triazol-1-yl]phenyl]methyl]-5-methyl-pyrazol-3-yl]-3-methyl-azetidin-3-yl]carbamate (6 mg, 0.0106 mmol) in DCM (2 mL) was added TFA (2 mL). The reaction mixture was stirred at 25° C. for 1 h. SM was consumed completely and the target was detected by LCMS. The solvent was removed under vacuum. The crude was purified by HPLC. 2-[4-[[3-(3-amino-3-methyl-azetidin-1-yl)-5-methyl-pyrazol-1-yl]methyl]phenyl]-4-[(2,6-difluorophenyl)methyl]-1,2,4-triazol-3-one (4.1 mg, 83.0% yield) was obtained as a white solid. MS (m/z): 466.2 [M+H]⁺. ¹H NMR (400 MHz, DMSO-d₆) δ 8.29 (s, 1H), 7.85-7.71 (m, 2H), 7.48 (tt, J=8.4, 6.7 Hz, 1H), 7.16 (dt, J=8.2, 3.8 Hz, 4H), 5.36 (s, 1H), 5.08 (s, 2H), 4.96 (s, 2H), 3.58-3.22 (m, 4H), 2.11 (s, 3H), 1.40 (s, 3H).

The synthesis procedure of Examples (Compounds) 538-541 was similar to the procedure of Example 537.

| Compound No. | | | Appearance | | MS(m/z) |
|---|---|---|---|---|---|
| Method | Activity | Structure and Name | and Yield | ¹H NMR Data | [M + H]⁺ |
| Example 537 Method 97 | ++ | 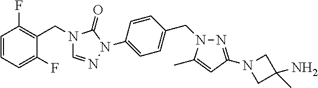 [SMILES: Cc1cc(N2CC(C)(N)C2)nn1Cc1ccc(-n2ncn(Cc3c(F)cccc3F)c2=O)cc1] | White solid, yield: 83.0% | ¹H NMR (400 MHZ, DMSO-d₆) δ 8.29 (s, 1H), 7.85-7.71 (m, 2H), 7.48 (tt, J = 8.4, 6.7 Hz, 1H), 7.16 (dt, J = 8.2, 3.8 Hz, 4H), 5.36 (s, 1H), 5.08 (s, 2H), 4.96 (s, 2H), 3.58-3.22 (m, 4H), 2.11 (s, 3H), | 466.2 |
| | | | | 1.40 (s, 3H). | |
| | | 2-(4-((3-(3-amino-3-methylazetidin- | | | |
| | | 1-yl)-5-methyl-1H-pyrazol-1- | | | |
| | | yl)methyl)phenyl)-4-(2,6-difluorobenzyl)- | | | |
| | | 2,4-dihydro-3H-1,2,4-triazol-3-one | | | |
| Example 538 Method 97 | ++++ | 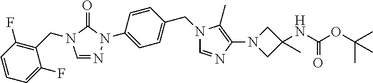 [SMILES: Cc1c(N2CC(C)(NC(=O)OC(C)(C)C)C2)ncn1Cc1ccc(-n2ncn(Cc3c(F)cccc3F)c2=O)cc1] | light green solid, yield: 5.7% | ¹H NMR (400 MHZ, Chloroform-d) δ 7.99 (d, J = 8.4 Hz, 2H), 7.59 (s, 1H), 7.50 (d, J = 14.4 Hz, 1H), 7.36 (ddd, J = 15.2, 8.4, 6.4 Hz, 1H), 7.13 (d, J = 8.4 Hz, 2H), 7.02-6.94 (m, 2H), 5.01-4.99 (m, 2H), 4.98- | 566.2 |
| | | | | 4.96 (m, 2H), 4.12 (brs, 2H), 3.91 | |
| | | tert-butyl | | (brs, 2H), 2.04 (s, 3H), 1.66 (s, 3H), | |
| | | (1-(1-(4-(4-(2,6-difluorobenzyl)-5- | | 1.43 (s, 9H). | |
| | | oxo-4,5-dihydro-1H-1,2,4-triazol- | | | |
| | | 1-yl)benzyl)-5-methyl-1H-imidazol- | | | |
| | | 4-yl)-3-methylazetidin-3-yl) | | | |
| | | carbamate | | | |
| Example 539 Method 97 | ++++ | 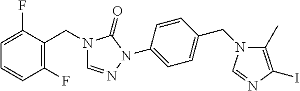 [SMILES: Cc1c(I)ncn1Cc1ccc(-n2ncn(Cc3c(F)cccc3F)c2=O)cc1] | White solid, yield: 1.67% | ¹H NMR (400 MHZ, Chloroform-d) δ 8.00 - 7.92 (m, 2H), 7.62-7.56 (m, 1H), 7.54 (s, 1H), 7.36 (tt, J = 8.4, 6.4 Hz, 1H), 7.15-7.08 (m, 2H), 7.03-6.93 (m, 2H), 5.08 (s, 2H), 4.97 (t, J = 1.2 Hz, 3H), 2.09 | 508.2 |
| | | | | (s, 3H). | |
| | | 4-(2,6-difluorobenzyl)-2-(4-((4-iodo- | | | |
| | | 5-methyl-1H-imidazol-1-yl)methyl)phenyl)- | | | |
| | | 2,4-dihydro-3H-1,2,4-triazol-3-one | | | |
| Example 540 Method 97 | ++++ | 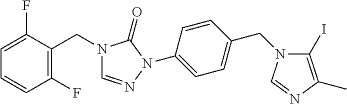 [SMILES: Cc1ncn(Cc2ccc(-n3ncn(Cc4c(F)cccc4F)c3=O)cc2)c1I] | White solid, yield: 0.3% | ¹H NMR (400 MHZ, Chloroform-d) δ 8.38 (s, 1H), 8.01 (d, J = 8.0 Hz, 2H), 7.59 (s, 1H), 7.36 (tt, J = 8.4, 6.4 Hz, 1H), 7.24 (d, J = 8.8 Hz, 3H), 7.03-6.92 (m, 2H), 5.17 (s, 2H), 4.97 (s, 2H), 2.34 (s, 3H). | 508.3 |
| | | 4-(2,6-difluorobenzyl)-2-(4-((5-iodo- | | | |
| | | 4-methyl-1H-imidazol-1-yl)methyl)phenyl)- | | | |
| | | 2,4-dihydro-3H-1,2,4-triazol-3-one | | | |
| Example 541 Method 97 | ++++ | 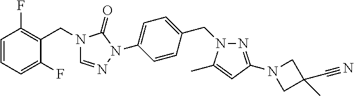 [SMILES: Cc1cc(N2CC(C)(C#N)C2)nn1Cc1ccc(-n2ncn(Cc3c(F)cccc3F)c2=O)cc1] | Off-white solid, yield: 14.5% | ¹H NMR (400 MHZ, DMSO-d₆) δ 8.29 (d, J = 4.2 Hz, 1H), 7.88-7.73 (m, 2H), 7.48 (tt, J =8.4, 6.6 Hz, 1H), 7.20-7.10 (m, 4H), 5.42 (d, J = 0.8 Hz, 1H), 5.10 (s, 2H), 4.97 (s, 2H), 3.99 (d, J = 7.5 Hz, 2H), 3.68 (d, J = 7.5 Hz, 2H), 2.12 (s, 3H), | 476.2 |
| | | | | 1.60 (s, 3H). | |
| | | 1-(1-(4-(4-(2,6-difluorobenzyl)-5- | | | |
| | | oxo-4,5-dihydro-1H-1,2,4-triazol- | | | |
| | | 1-yl)benzyl)-5-methyl-1H-pyrazol- | | | |
| | | 3-yl)-3-methylazetidine-3-carbonitrile | | | |

### Example 542: Method 98

### 4-(2,6-difluorobenzyl)-2-(4-((2-(2,5-dihydro-1H-pyrrol-3-yl)-4-methylthiazol-5-yl)oxy)phenyl)-2,4-dihydro-3H-1,2,4-triazol-3-one

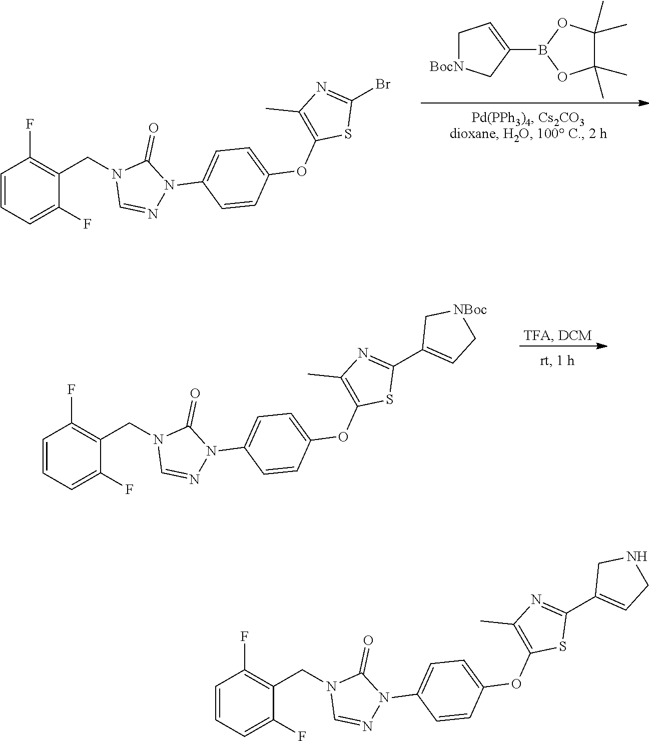

### Step 1: tert-butyl 3-(5-(4-(4-(2,6-difluorobenzyl)-5-oxo-4,5-dihydro-1H-1,2,4-triazol-1-yl) phenoxy)-4-methylthiazol-2-yl)-2,5-dihydro-1H-pyrrole-1-carboxylate

To a solution of 2-[4-(2-bromo-4-methyl-thiazol-5-yl)oxyphenyl]-4-[(2,6-difluorophenyl)methyl]-1,2,4-triazol-3-one (500 mg, 1.04 mmol) in 1,4-dioxane (4 mL) and H₂O (1 mL) were added tert-butyl 3-(4,4,5,5-tetramethyl-1,3,2-dioxaborolan-2-yl)-2,5-dihydropyrrole-1-carboxylate (462 mg, 1.56 mmol), tetrakis(triphenylphosphine)palladium (362 mg, 0.313 mmol) and Cs₂CO₃ (848 mg, 2.61 mmol) under Ar. The reaction mixture was stirred at 100° C. for 2 h. The target product was detected by LCMS. The reaction mixture was taken up in EtOAc (200 mL) and the organics were washed with 2×100 mL water and then 1×100 mL saturated brine solution. The organics were then separated and dried (MgSO₄) before concentration to dryness. The crude was then purified by flash column chromatography eluted with 10-60% EtOAc in PE. The desired fractions were concentrated to dryness in vacuo. Tert-butyl 3-[5-[4-[4-[(2,6-difluorophenyl)methyl]-5-oxo-1,2,4-triazol-1-yl]phenoxy]-4-methyl-thiazol-2-yl]-2,5-dihydropyrrole-1-carboxylate (326 mg, 55.1% yield) was obtained as an off-white solid. MS (m/z): 568.2 [M+H]⁺. ¹H NMR (400 MHz, DMSO) δ 8.30 (s, 1H), 7.86 (d, J=9.0 Hz, 2H), 7.48 (tt, J=8.5, 6.6 Hz, 1H), 7.30-7.06 (m, 4H), 6.46 (dt, J=11.6, 2.2 Hz, 1H), 4.97 (s, 2H), 4.40 (dd, J=10.7, 5.7 Hz, 2H), 4.21 (h, J=3.5 Hz, 2H), 2.20 (s, 3H), 1.44 (d, J=4.9 Hz, 9H).

### Step 2: 4-(2,6-difluorobenzyl)-2-(4-((2-(2,5-dihydro-1H-pyrrol-3-yl)-4-methylthiazol-5-yl)oxy)phenyl)-2,4-dihydro-3H-1,2,4-triazol-3-one

To a solution of tert-butyl 3-[5-[4-[4-[(2,6-difluorophenyl)methyl]-5-oxo-1,2,4-triazol-1-yl]phenoxy]-4-methyl-thiazol-2-yl]-2,5-dihydropyrrole-1-carboxylate (50 mg, 0.0881 mmol) in DCM (2 mL) was added TFA (2 mL). The reaction mixture was stirred at 25° C. for 1 h. SM was consumed completely and the target product was detected by LCMS. The crude was purified by reversed-phase column. 4-[(2,6-difluorophenyl) methyl]-2-[4-[2-(2,5-dihydro-1H-pyrrol-3-yl)-4-methyl-thiazol-5-yl]oxyphenyl]-1,2,4-triazol-3-one (36 mg, 87.4% yield) was obtained as a brown solid. MS (m/z): 468.2 [M+H]⁺. ¹H NMR (400 MHz, DMSO-d₆) δ 8.31 (s, 1H), 7.92-7.77 (m, 2H), 7.49 (tt, J=8.5, 6.6 Hz, 1H), 7.26-7.07 (m, 4H), 6.53 (p, J=2.2 Hz, 1H), 4.98 (s, 2H), 4.36 (dq, J=6.2, 3.0 Hz, 2H), 4.17 (dq, J=5.7, 2.8 Hz, 2H), 2.22 (s, 3H).

The synthesis procedure of Examples (Compounds) 543-548 were similar to the procedure of Example 542.

| Compound No. | | | Appearance | | MS(m/z) |
|---|---|---|---|---|---|
| Method | Activity | Structure and Name | and Yield | ¹H NMR Data | [M + H]⁺ |
| Example 542 Method 98 | ++++ | 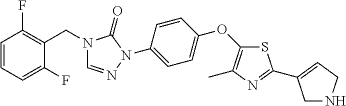 [SMILES: Cc1nc(C2=CCNC2)sc1Oc1ccc(-n2ncn(Cc3c(F)cccc3F)c2=O)cc1] | brownsolid, yield: 87.4% | ¹H NMR (400 MHZ, DMSO-d₆) δ 8.31 (s, 1H), 7.92-7.77 (m, 2H), 7.49 (tt, J = 8.5, 6.6 Hz, 1H), 7.26-7.07 (m, 4H), 6.53 (p, J = 2.2 Hz, 1H), 4.98 (s, 2H), 4.36 (dq, J = 6.2, 3.0 Hz, 2H), 4.17 (dq, J = 5.7, 2.8 Hz, 2H), 2.22 (s, 3H). | 468.2 |
| | | 4-(2,6-difluorobenzyl)-2-(4-((2-(2,5- | | | |
| | | dihydro-1H-pyrrol-3-yl)-4- | | | |
| | | methylthiazol-5-yl)oxy)phenyl)-2,4- | | | |
| | | dihydro-3H-1,2,4-triazol-3-one | | | |
| Example 543 Method 97 | ++ | 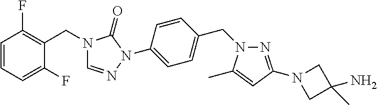 [SMILES: Cc1cc(N2CC(C)(N)C2)nn1Cc1ccc(-n2ncn(Cc3c(F)cccc3F)c2=O)cc1] | White solid, yield: 83.0% | ¹H NMR (400 MHZ, DMSO-d₆) δ 8.29 (s, 1H), 7.85-7.71 (m, 2H), 7.48 (tt, J = 8.4, 6.7 Hz, 1H), 7.16 (dt, J = 8.2, 3.8 Hz, 4H), 5.36 (s, 1H), 5.08 (s, 2H), 4.96 (s, 2H), 3.58-3.22 (m, 4H), 2.11 (s, 3H), 1.40 (s, 3H). | 466.2 |
| | | 2-(4-((3-(3-amino-3-methylazetidin- | | | |
| | | 1-yl)-5-methyl-1H-pyrazol-1- | | | |
| | | yl)methyl)phenyl)-4-(2,6-difluorobenzyl)- | | | |
| | | 2,4-dihydro-3H-1,2,4-triazol-3-one | | | |
| Example 544 Method 98 | ++++ | 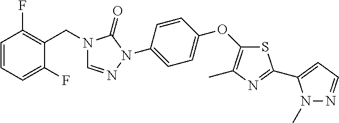 [SMILES: Cc1nc(-c2ccnn2C)sc1Oc1ccc(-n2ncn(Cc3c(F)cccc3F)c2=O)cc1] | Off-white solid , yield: 9.98% | ¹H NMR (400 MHZ, DMSO-d₆) δ 8.30 (d, J = 0.9 Hz, 1H), 7.92-7.83 (m, 2H), 7.55-7.45 (m, 2H), 7.33-7.21 (m, 2H), 7.16 (t, J = 8.1 Hz, 2H), 6.72 (d, J = 2.0 Hz, 1H), 4.97 (s, 2H), 4.15 (s, 3H), 2.28 (s, 3H). | 481.1 |
| | | 4-(2,6-difluorobenzyl)-2-(4-((4-methyl- | | | |
| | | 2-(1-methyl-1H-pyrazol-5- | | | |
| | | yl)thiazol-5-yl)oxy)phenyl)-2,4-dihydro- | | | |
| | | 3H-1,2,4-triazol-3-one | | | |
| Example 545 Method 98 | ++++ | 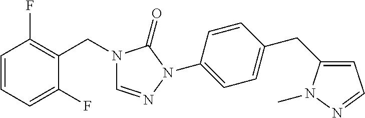 [SMILES: Cn1nccc1Cc1ccc(-n2ncn(Cc3c(F)cccc3F)c2=O)cc1] | yellow solid, yield: 55.8% | ¹H NMR (400 MHZ, DMSO-d₆) δ 8.29 (s, 1H), 7.87-7.74 (m, 2H), 7.48 (tt, J = 8.4, 6.6 Hz, 1H), 7.34-7.23 (m, 3H), 7.20-7.10 (m, 2H), 5.97 (d, J = 1.8 Hz, 1H), 4.97 (s, 2H), 4.03 (s, 2H), 3.67 (s, 3H). | 382.2 |
| | | 4-(2,6-difluorobenzyl)-2-(4-((1-methyl- | | | |
| | | 1H-pyrazol-5-yl)methyl)phenyl)- | | | |
| | | 2,4-dihydro-3H-1,2,4-triazol-3-one | | | |
| Example 546 Method 98 | ++++ | 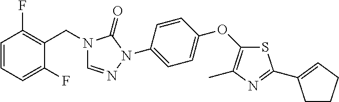 [SMILES: Cc1nc(C2=CCCC2)sc1Oc1ccc(-n2ncn(Cc3c(F)cccc3F)c2=O)cc1] | white solid, yield: 20.6% | NA | 467.1 |
| | | 2-(4-((2-(cyclopent-1-en-1-yl)-4- | | | |
| | | methylthiazol-5-yl)oxy)phenyl)-4- | | | |
| | | (2,6-difluorobenzyl)-2,4-dihydro- | | | |
| | | 3H-1,2,4-triazol-3-one | | | |
| Example 547 Method 98 | ++++ | 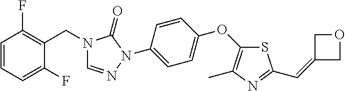 [SMILES: Cc1nc(C=C2COC2)sc1Oc1ccc(-n2ncn(Cc3c(F)cccc3F)c2=O)cc1] | white solid, yield: 47.1% | ¹H NMR (400 MHZ, DMSO-d₆) δ 8.28 (d, J = 2.5 Hz, 1H), 7.84 (dd, J = 9.3, 2.7 Hz, 2H), 7.48 (tt, J = 8.5, 6.6 Hz, 1H), 7.24-7.08 (m, 4H), 6.45 (t, J = 2.4 Hz, 1H), 5.42-5.20 (m, 4H), 4.97 (s, 2H), 2.18 (s, 3H). | 469.1 |
| | | 4-(2,6-difluorobenzyl)-2-(4-((4-methyl- | | | |
| | | 2-(oxetan-3-ylidenemethyl)thiazol- | | | |
| | | 5-yl)oxy)phenyl)-2,4-dihydro- | | | |
| | | 3H-1,2,4-triazol-3-one | | | |
| Example 548 Method 98 | ++++ | 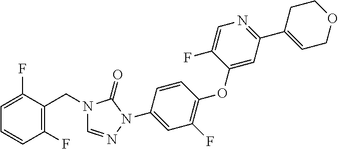 [SMILES: O=c1n(Cc2c(F)cccc2F)cnn1-c1ccc(Oc2cc(C3=CCOCC3)ncc2F)c(F)c1] | white solid, yield: 27.4% | ¹H NMR (400 MHZ, CDCl₃) δ 8.42 (s, 1H), 8.00 (m, 1H), 7.88 (d, J = 8.7 Hz, 1H), 7.63 (s, 1H), 7.43-7.34 (m, 1H), 7.23 (d, J = 8.7 Hz, 1H), 7.00 (m, 2H), 6.74 (d, J = 6.4 Hz, 1H), 6.45 (s, 1H), 5.00 (s, 2H), 4.29 (d, J = 1.6 Hz, 2H), 3.87 (s, 2H), 2.48 (s, 2H). | 499.2 |
| | | 4-(2,6-difluorobenzyl)-2-(4-((2-(3,6- | | | |
| | | dihydro-2H-pyran-4-yl)-5-fluoropyridin- | | | |
| | | 4-yl)oxy)-3-fluorophenyl)-2,4- | | | |
| | | dihydro-3H-1,2,4-triazol-3-one | | | |

### Example 549: Method 99

### 4-(2,6-difluorobenzyl)-2-(4-((4-methyl-2-(pyrrolidin-3-yl)thiazol-5-yl)oxy)phenyl)-2,4-dihydro-3H-1,2,4-triazol-3-one

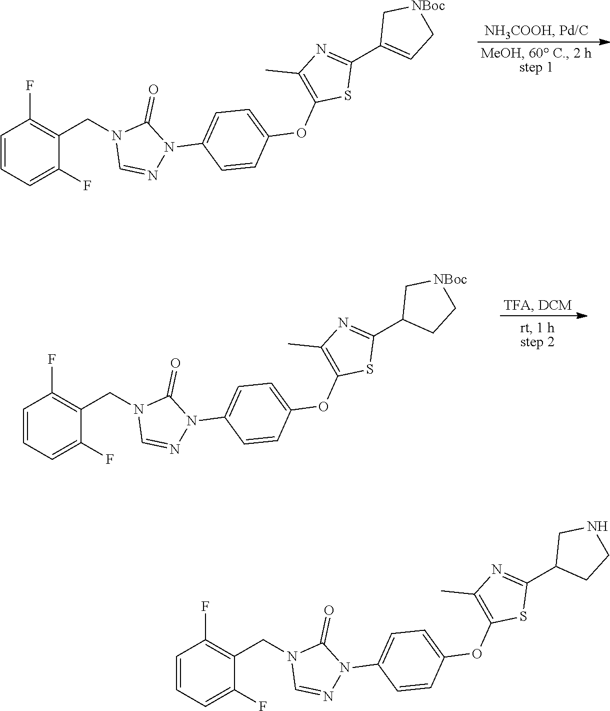

### Step 1: tert-butyl 3-(5-(4-(4-(2,6-difluorobenzyl)-5-oxo-4,5-dihydro-1H-1,2,4-triazol-1-yl)phenoxy)-4-methylthiazol-2-yl)pyrrolidine-1-carboxylate

To a solution of tert-butyl 3-[5-[4-[4-[(2,6-difluorophenyl)methyl]-5-oxo-1,2,4-triazol-1-yl]phenoxy]-4-methyl-thiazol-2-yl]-2,5-dihydropyrrole-1-carboxylate (200 mg, 0.352 mmol) in methanol (8 mL) were added ammonium formate (111 mg, 1.76 mmol) and palladium (150 mg, 0.0705 mmol) under Ar. The reaction mixture was stirred at 60° C. for 2 h. The target product was detected by LCMS. The reaction mixture was concentrated to dryness and the residue was taken up in EtOAc (100 mL) and the organic layers were washed with 2×100 mL water and then 1×100 mL saturated brine solution. The organics were then separated and dried (MgSO₄) before concentration to dryness. The crude was then purified by reversed-phase column. The desired fractions were concentrated to dryness under vacuum. Tert-butyl 3-[5-[4-[4-[(2,6-difluorophenyl)methyl]-5-oxo-1,2,4-triazol-1-yl]phenoxy]-4-methyl-thiazol-2-yl]pyrrolidine-1-carboxylate (137 mg, 68.3% yield) was obtained as a white solid. MS (m/z): 570.2 [M+H]⁺.

### Step 2: 4-(2,6-difluorobenzyl)-2-(4-((4-methyl-2-(pyrrolidin-3-yl)thiazol-5-yl)oxy)phenyl)-2,4-dihydro-3H-1,2,4-triazol-3-one

To a solution of tert-butyl 3-[5-[4-[4-[(2,6-difluorophenyl)methyl]-5-oxo-1,2,4-triazol-1-yl]phenoxy]-4-methyl-thiazol-2-yl]pyrrolidine-1-carboxylate (50 mg, 0.263 mmol) in DCM (4 mL) was added TFA (1.0 mL, 13.2 mmol). The reaction mixture was stirred at 25° C. for 2 h. The reaction mixture was concentrated to dryness and the residue was purified by reversed-phase column. 4-[(2,6-difluorophenyl)methyl]-2-[4-(4-methyl-2-pyrrolidin-3-yl-thiazol-5-yl)oxyphenyl]-1,2,4-triazol-3-one (86 mg, 69.6% yield) was obtained as a white solid. MS (m/z): 470.2 [M+H]⁺. ¹H NMR (400 MHz, DMSO-d₆) δ 8.30 (s, 1H), 7.93-7.74 (m, 2H), 7.49 (tt, J=8.5, 6.6 Hz, 1H), 7.26-7.07 (m, 4H), 4.97 (s, 2H), 3.84 (p, J=7.7 Hz, 1H), 3.60 (dd, J=11.6, 8.0 Hz, 1H), 3.46-3.20 (m, 3H), 2.38 (dtd, J=13.0, 7.5, 5.6 Hz, 1H), 2.18 (s, 3H), 2.15-2.06 (m, 1H).

The synthesis procedure of Examples (Compounds) 550-553 were similar to the procedure of Example 549.

| Compound No. | | | Appearance | | MS(m/z) |
|---|---|---|---|---|---|
| Method | Activity | Structure and Name | and Yield | ¹H NMR Data | [M + H]⁺ |
| Example 549 Method 99 | ++++ | 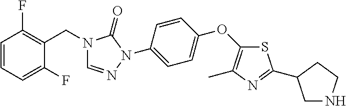 [SMILES: Cc1nc(C2CCNC2)sc1Oc1ccc(-n2ncn(Cc3c(F)cccc3F)c2=O)cc1] | White solid, yield: 69.6% | ¹H NMR (400 MHZ, DMSO-d₆) δ 8.30 (s, 1H), 7.93-7.74 (m, 2H), 7.49 (tt, J = 8.5, 6.6 Hz, 1H), 7.26-7.07 (m, 4H), 4.97 (s, 2H), 3.84 (d, J = 7.7 Hz, 1H), 3.60 (dd, J = 11.6, 8.0 Hz, 1H), 3.46-3.20 (m, 3H), 2.38 (dtd, J = 13.0, 7.5, 5.6 Hz, | 470.2 |
| | | | | 1H), 2.18 (s, 3H), 2.15-2.06 (m,1H). | |
| | | 4-(2,6-difluorobenzyl)-2-(4-((4-methyl- | | | |
| | | 2-(pyrrolidin-3-yl)thiazol- | | | |
| | | 5-yl)oxy)phenyl)-2,4-dihydro-3H-1, | | | |
| | | 2,4-triazol-3-one | | | |
| Example 550 Method 99 | ++++ | 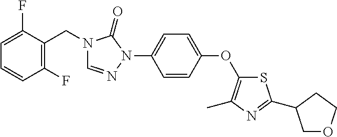 [SMILES: Cc1nc(C2CCOC2)sc1Oc1ccc(-n2ncn(Cc3c(F)cccc3F)c2=O)cc1] | white solid, yield: 22.9% | ¹H NMR (400 MHZ, DMSO-d₆) δ 8.29 (s, 1H), 7.88-7.80 (m, 2H), 7.48 (tt, J = 8.4, 6.7 Hz, 1H), 7.21- 7.10 (m, 4H), 4.97 (s, 2H), 4.00 (dd, J = 8.4, 7.2 Hz, 1H), 3.87 (td, J = 8.4, 5.6 Hz, 1H), 3.82-3.65 (m, 3H), 2.39-2.26 (m, 1H), 2.15 (s, 3H), 2.14-2.05 (m, 1H). | 471.2 |
| | | 4-(2,6-difluorobenzyl)-2-(4-((4-methyl- | | | |
| | | 2-(tetrahydrofuran-3-yl)thiazol- | | | |
| | | 5-yl)oxy)phenyl)-2,4-dihydro- | | | |
| | | 3H-1,2,4-triazol-3-one | | | |
| Example 551 | ++++ | 4-(2,6-difluorobenzyl)-2-(4-((4-methyl- | White solid, | ¹H NMR (400 MHZ, Chloroform-d) | 485.2 |
| Method 99 | | 2-(tetrahydro-2H-pyran-4- | yield: 17.3% | δ 7.94-7.86 (m, 2H), 7.56 (s, 1H), | |
| | | yl)thiazol-5-yl)oxy)phenyl)-2,4-dihydro- | | 7.36 (tt, J = 8.4, 6.4 Hz, 1H), 7.07- | |
| | | 3H-1,2,4-triazol-3-one | | 6.92 (m, 4H), 4.97 (d, J = 1.2 Hz, | |
| | | | | 2H), 4.05 (ddd, J = 11.6, 4.4, 2.0 | |
| | | | | Hz, 2H), 3.52 (td, J = 11.6, 2.0 Hz, | |
| | | | | 2H), 3.16 (d, J = 12.0 Hz, 1H), 2.25 | |
| | | | | (s, 3H), 2.07-2.00 (m, 2H), 1.91- | |
| | | | | 1.76 (m, 2H). | |
| Example 552 Method 99 | ++++ | 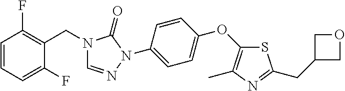 [SMILES: Cc1nc(CC2COC2)sc1Oc1ccc(-n2ncn(Cc3c(F)cccc3F)c2=O)cc1] | Off-white solid, yield: 16.6% | ¹H NMR (400 MHZ, DMSO-d₆) δ 8.28 (s, 1H), 7.92-7.75 (m, 2H), 7.48 (ddd, J = 15.0, 8.5, 6.7 Hz, 1H), 7.25-7.04 (m, 4H), 4.97 (s, 2H), 4.69 (dd, J = 7.7, 6.0 Hz, 2H), 4.37 (t, J = 6.1 Hz, 2H), 3.38-3.28 | 471.1 |
| | | | | (m, 1H), 3.24 (d, J = 7.7 Hz, 2H), | |
| | | 4-(2,6-difluorobenzyl)-2-(4-((4-methyl- | | 2.13 (s, 3H). | |
| | | 2-(oxetan-3-ylmethyl)thiazol- | | | |
| | | 5-yl)oxy)phenyl)-2,4-dihydro-3H- | | | |
| | | 1,2,4-triazol-3-one | | | |
| Example 553 Method 99 | +++ | 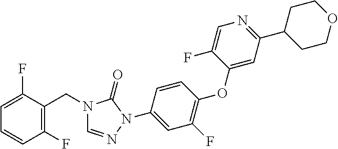 [SMILES: O=c1n(Cc2c(F)cccc2F)cnn1-c1ccc(Oc2cc(C3CCOCC3)ncc2F)c(F)c1] | white solid, yield: 28.7% | ¹H NMR (400 MHZ, CDCl₃) δ 8.39 (d, J = 2.1 Hz, 1H), 8.00 (m, 1H), 7.88 (d, J = 9.0 Hz, 1H), 7.63 (s, 1H), 7.43-7.35 (m, 1H), 7.24 (m, 1H), 7.00 (m, 2H), 6.53 (d, J = 6.5 Hz, 1H), 5.00 (s, 2H), 4.02 (m, 2H), 3.50-3.41 (m, 2H), 2.87-2.76 (m, 1H), 1.74 (m, 4H). | 501.2 |
| | | 4-(2,6-difluorobenzyl)-2-(3-fluoro- | | | |
| | | 4-((5-fluoro-2-(tetrahydro-2H-pyran- | | | |
| | | 4-yl)pyridin-4-yl)oxy)phenyl)- | | | |
| | | 2,4-dihydro-3H-1,2,4-triazol-3-one | | | |

### Example 554: Method 100

### 4-(2,6-difluorobenzyl)-2-(4-((4-methyl-2-(1-methylpyrrolidin-3-yl)thiazol-5-yl)oxy)phenyl)-2,4-dihydro-3H-1,2,4-triazol-3-one

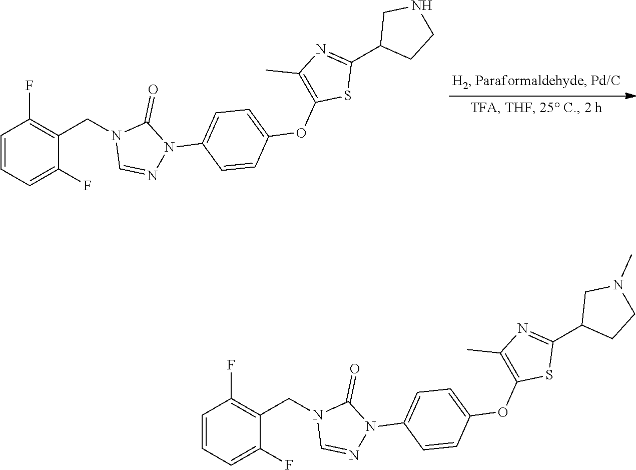 [SMILES: Cc1nc(C2CCNC2)sc1Oc1ccc(-n2ncn(Cc3c(F)cccc3F)c2=O)cc1 ; Cc1nc(C2CCN(C)C2)sc1Oc1ccc(-n2ncn(Cc3c(F)cccc3F)c2=O)cc1]

To a solution of 4-[(2,6-difluorophenyl)methyl]-2-[4-(4-methyl-2-pyrrolidin-3-yl-thiazol-5-yl)oxyphenyl]-1,2,4-triazol-3-one (50 mg, 0.106 mmol) in THF (10 mL) were added paraformaldehyde (19 mg, 0.213 mmol) and Pd/C (20 mg), TFA (a drop) under Ar. The reaction mixture was stirred at 25° C. for 2 h under H₂ (15 psi). The target product was detected by LCMS. The reaction mixture was filtered through a pad of Celite and the pad was washed with EtOH (50 mL). The combined filtrates were concentrated to dryness to give a crude product. The crude was purified by reversed-phase column. 4-[(2,6-difluorophenyl)methyl]-2-[4-[4-methyl-2-(1-methylpyrrolidin-3-yl)thiazol-5-yl]oxyphenyl]-1,2,4-triazol-3-one (18 mg, 35.0% yield) was obtained as a white solid. MS (m/z): 484.1 [M+H]⁺.1H NMR (400 MHz, DMSO-d₆) δ 8.29 (s, 1H), 7.90-7.75 (m, 2H), 7.48 (tt, J=8.5, 6.7 Hz, 1H), 7.23-7.07 (m, 4H), 4.97 (s, 2H), 3.58 (ddt, J=10.3, 7.4, 5.4 Hz, 1H), 2.75 (dd, J=9.2, 7.4 Hz, 1H), 2.62 (td, J=8.8, 5.3 Hz, 2H), 2.44 (td, J=8.5, 6.2 Hz, 1H), 2.26 (s, 3H), 2.13 (s, 3H), 2.32-2.20 (m, 1H), 1.96 (tdd, J=11.9, 9.2, 6.6 Hz, 1H).H), 3.52 (dd, J=8.0, 5.9 Hz, 1H), 2.06-1.89 (m, 1H), 0.97 (d, J=6.2 Hz, 3H).

| Compound No. | | | Appearance | | MS(m/z) |
|---|---|---|---|---|---|
| Method | Activity | Structure and Name | and Yield | ¹H NMR Data | [M + H]⁺ |
| Example 554 Method 100 | ++++ | 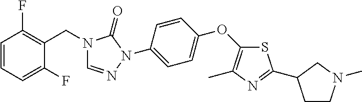 [SMILES: Cc1nc(C2CCN(C)C2)sc1Oc1ccc(-n2ncn(Cc3c(F)cccc3F)c2=O)cc1] | White solid, yield: 35.0% | ¹H NMR (400 MHZ, DMSO-d₆) δ 8.29 (s, 1H), 7.90-7.75 (m, 2H), 7.48 (tt, J = 8.5, 6.7 Hz, 1H), 7.23-7.07 (m, 4H), 4.97 (s, 2H), 3.58 (ddt, J = 10.3, 7.4, 5.4 Hz, 1H), 2.75 (dd, J = 9.2, 7.4 Hz, 1H), 2.62 (td, J = 8.8, 5.3 Hz, 2H), 2.44 (td, | 484.1 |
| | | | | J = 8.5, 6.2 Hz, 1H), 2.26 (s, 3H), | |
| | | 4-(2,6-difluorobenzyl)-2-(4-((4- | | 2.13 (s, 3H), 2.32-2.20 (m, 1H), | |
| | | methyl-2-(1-methylpyrrolidin-3-yl) | | 1.96 (tdd, J = 11.9, 9.2, 6.6 Hz, 1H). | |
| | | thiazol-5-yl)oxy)phenyl)-2,4-dihydro- | | | |
| | | 3H-1,2,4-triazol-3-one | | | |

### Example 555: Method 101

### 2-(4-((2-(3-(aminomethyl)-3-methylazetidin-1-yl)-5-fluoropyridin-4-yl)oxy)-3-fluorophenyl)-4-(2,6-difluorobenzyl)-2,4-dihydro-3H-1,2,4-triazol-3-one

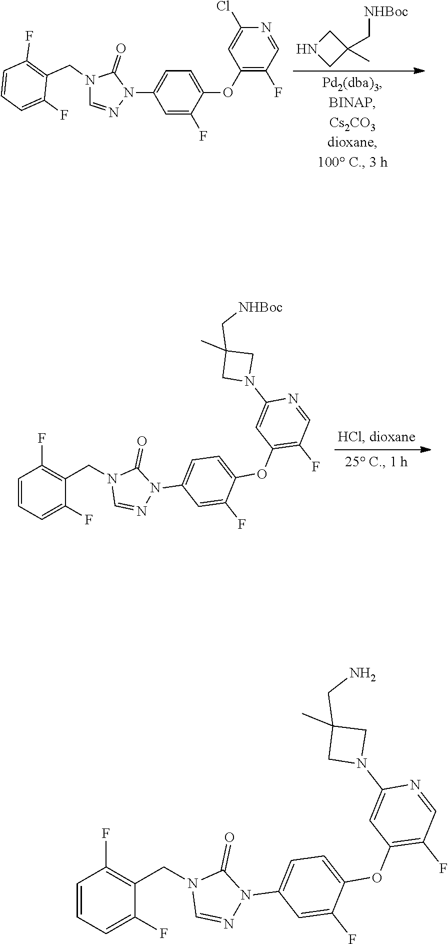

### Step 1: tert-butyl ((1-(4-(4-(4-(2,6-difluorobenzyl)-5-oxo-4,5-dihydro-1H-1,2,4-triazol-1-yl)-2-fluorophenoxy)-5-fluoropyridin-2-yl)-3-methylazetidin-3-yl)methyl)carbamate

To a solution of 2-[4-[(2-chloro-5-fluoro-4-pyridyl)oxy]-3-fluoro-phenyl]-4-[(2,6-difluorophenyl)methyl]-1,2,4-triazol-3-one (80 mg, 0.177 mmol) in 1,4-dioxane (4 mL) were added tert-butyl N-[(3-methylazetidin-3-yl)methyl]carbamate (53 mg, 0.266 mmol), Pd₂(dba)₃ (65 mg, 0.0710 mmol), BINAP (66 mg, 0.106 mmol) and cesium carbonate (173 mg, 0.532 mmol) under Ar. The reaction mixture was stirred at 100° C. for 3 h. The target product was detected by LCMS. The reaction mixture was concentrated to dryness and the residue was taken up in EtOAc (100 mL) and the organic layers were washed with 2×100 mL water and then 1×100 mL saturated brine solution. The organics were then separated and dried (MgSO₄) before concentration to dryness. The crude was then purified by reversed-phase column. The desired fractions were concentrated to dryness in vacuo. Tert-butyl N-[[1-[4-[4-[4-[(2,6-difluorophenyl)methyl]-5-oxo-1,2,4-triazol-1-yl]-2-fluoro-phenoxy]-5-fluoro-2-pyridyl]-3-methyl-azetidin-3-yl]methyl]carbamate (63 mg, 57.8% yield) was obtained as a white solid. MS (m/z): 615.2 [M+H]⁺. ¹H NMR (400 MHz, DMSO-d₆) δ 8.40 (s, 1H), 8.10 (d, J=3.0 Hz, 1H), 7.93 (dd, J=12.4, 2.5 Hz, 1H), 7.79 (ddd, J=9.0, 2.6, 1.3 Hz, 1H), 7.56-7.40 (m, 2H), 7.23-7.13 (m, 2H), 7.06 (t, J=6.3 Hz, 1H), 5.61 (d, J=5.6 Hz, 1H), 5.00 (s, 2H), 3.62 (d, J=7.9 Hz, 2H), 3.03 (d, J=6.3 Hz, 2H), 1.35 (s, 9H), 1.15 (s, 3H).

### Step 2: 2-(4-((2-(3-(aminomethyl)-3-methylazetidin-1-yl)-5-fluoropyridin-4-yl)oxy)-3-fluorophenyl)-4-(2,6-difluorobenzyl)-2,4-dihydro-3H-1,2,4-triazol-3-one

To a solution of tert-butyl N-[[1-[4-[4-[4-[(2,6-difluorophenyl)methyl]-5-oxo-1,2,4-triazol-1-yl]-2-fluoro-phenoxy]-5-fluoro-2-pyridyl]-3-methyl-azetidin-3-yl]methyl]carbamate (30 mg, 0.0488 mmol) in 1,4-dioxane (2 mL) was added hydrochloric acid (0.5 mL, 4 N). The reaction mixture was stirred at 25° C. for 1 h. The target product was detected by LCMS. The reaction mixture was concentrated to dryness and the residue was purified by reversed-phase column. 2-[4-[[2-[3-(aminomethyl)-3-methyl-azetidin-1-yl]-5-fluoro-4-pyridyl]oxy]-3-fluoro-phenyl]-4-[(2,6-difluorophenyl)methyl]-1,2,4-triazol-3-one (11 mg, 43.8% yield) was obtained as a white solid. MS (m/z): 515.2 [M+H]⁺. ¹H NMR (400 MHz, CDCl₃) δ 8.47-8.25 (m, 5H), 7.96 (dd, J=12.3, 2.5 Hz, 1H), 7.82 (ddd, J=9.0, 2.6, 1.3 Hz, 1H), 7.66-7.42 (m, 2H), 7.24-7.05 (m, 2H), 5.78 (d, J=6.3 Hz, 1H), 5.00 (s, 2H), 3.97 (d, J=9.2 Hz, 2H), 3.70 (d, J=9.1 Hz, 2H), 3.01 (q, J=5.8 Hz, 2H), 1.31 (s, 2H).

The synthesis procedure of Examples (Compounds) 556-557 was similar to the procedure of Example 555.

| Compound | | | | | |
|---|---|---|---|---|---|
| No. | | | Appearance | | MS(m/z) |
| Method | Activity | Structure and Name | and Yield | ¹H NMR Data | [M + H]⁺ |
| Example 555 Method 101 | ++++ | 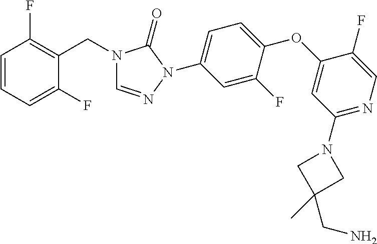 [SMILES: CC1(CN)CN(c2cc(Oc3ccc(-n4ncn(Cc5c(F)cccc5F)c4=O)cc3F)c(F)cn2)C1] | white solid, 43.8% yield. | ¹H NMR (400 MHZ, CDCl₃) δ 8.47-8.25 (m, 2H), 7.96 (dd, J = 12.3, 2.5 Hz, 1H), 7.82 (ddd, J = 9.0, 2.6, 1.3 Hz, 1H), 7.66-7.42 (m, 2H), 7.24-7.05 (m, 2H), 5.78 (d, J = 6.3 Hz, 1H), 5.00 (s, 2H), 3.97 (d, J = 9.2 Hz, 2H), 3.70 (d, J = 9.1 Hz, 2H), 3.01 (q, J = 5.8 Hz, 2H), 1.31 (s, 3H). | 515.2 |
| | | 2-(4-((2-(3-(aminomethyl)-3- | | | |
| | | methylazetidin-1-yl)-5-fluoropyridin- | | | |
| | | 4-yl)oxy)-3-fluorophenyl)-4-(2,6- | | | |
| | | difluorobenzyl)-2,4-dihydro-3H-1,2,4- | | | |
| | | triazol-3-one | | | |
| Example 556 Method 101 | ++++ | 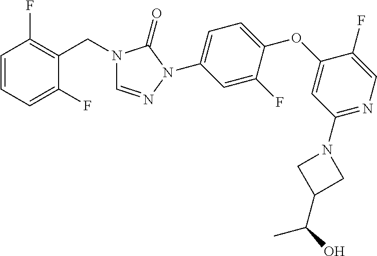 [SMILES: C[C@H](O)C1CN(c2cc(Oc3ccc(-n4ncn(Cc5c(F)cccc5F)c4=O)cc3F)c(F)cn2)C1] | white solid, yield: 6.1% | ¹H NMR (400 MHZ, DMSO-d₆) δ 8.39 (s, 1H), 8.10 (d, J = 3.0 Hz, 1H), 7.93 (dd, J = 12.4, 2.5 Hz, 1H), 7.78 (ddd, J = 9.0, 2.6, 1.3 Hz, 1H), 7.48 (td, J = 8.7, 5.6 Hz, 2H), 7.28-7.04 (m, 2H), 5.65 (d, J = 5.6 Hz, 1H), 5.00 (s, 2H), 4.67 (d, J = 4.9 Hz, 1H), 3.83-3.72 (m, 2H), 3.72-3.60 (m, 2H), 3.52 (dd, J = 8.0, 5.9 Hz, 1H), 2.06-1.89 (m, 1H), 0.97 (d, J = 6.2 Hz, 3H). | 516.1 |
| | | (S)-4-(2,6-difluorobenzyl)-2-(3- | | | |
| | | fluoro-4-((5-fluoro-2-(3-(1- | | | |
| | | hydroxyethyl)azetidin-1-yl)pyridin- | | | |
| | | 4-yl)oxy)phenyl)-2,4-dihydro-3H- | | | |
| | | 1,2,4-triazol-3-one | | | |
| Example 557 Method 101 | ++++ | 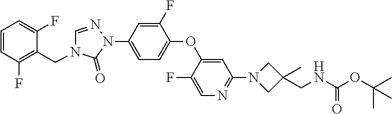 [SMILES: CC1(CNC(=O)OC(C)(C)C)CN(c2cc(Oc3ccc(-n4ncn(Cc5c(F)cccc5F)c4=O)cc3F)c(F)cn2)C1] | white solid, yield: 57.8% | ¹H NMR (400 MHZ, DMSO-d₆) δ 8.40 (s, 1H), 8.10 (d, J = 3.0 Hz, 1H), 7.93 (dd, J = 12.4, 2.5 Hz, 1H), 7.79 (ddd, J = 9.0, 2.6, 1.3 Hz, 1H), 7.56-7.40 (m, 2H), 7.23-7.13 (m, 1H), 7.06 (t, J = 6.3 Hz, 1H), 5.61 (d, J = 5.6 Hz, 1H), 5.00 (s, 2H), 3.97 (d, J = 8.0 Hz, 2H), 3.62 (d, J = 7.9 Hz, 2H), 3.03 (d, J = 6.3 Hz, 2H), 1.35 (s, 9H), 1.15 (s, 3H). | 615.2 |
| | | tert-butyl | | | |
| | | ((1-(4-(4-(4-(2,6-difluorobenzyl)- | | | |
| | | 5-oxo-4,5-dihydro-1H-1,2,4-triazol- | | | |
| | | 1-yl)-2-fluorophenoxy)-5-fluoropyridin- | | | |
| | | 2-yl)-3-methylazetidin-3-yl)methyl)carbamate | | | |

### Example 558: Method 102

### 2-(4-((2-cyclopentyl-4-methylthiazol-5-yl)oxy)phenyl)-4-(2,6-difluorobenzyl)-2,4-dihydro-3H-1,2,4-triazol-3-one

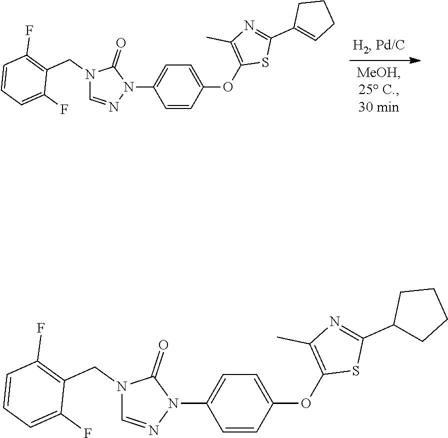 [SMILES: Cc1nc(C2=CCCC2)sc1Oc1ccc(-n2ncn(Cc3c(F)cccc3F)c2=O)cc1 ; Cc1nc(C2CCCC2)sc1Oc1ccc(-n2ncn(Cc3c(F)cccc3F)c2=O)cc1]

To a solution of 2-[4-[2-(cyclopenten-1-yl)-4-methyl-thiazol-5-yl]oxyphenyl]-4-[(2,6-difluorophenyl)methyl]-1,2,4-triazol-3-one (60 mg, 0.129 mmol) in Methanol (10 mL) was added Palladium (4.1 mg, 0.0386 mmol) under H₂ (15 Psi). The reaction mixture was stirred at 25° C. for 30 min. The target product was detected by LCMS. The suspension was filtered through a pad of Celite and the filter cake was washed with EtOH (100 mL). The combined filtrates were concentrated under vacuum. The crude was purified by flash column chromatography eluted with 10-50% EtOAc in PE. 2-[4-(2-cyclopentyl-4-methyl-thiazol-5-yl)oxyphenyl]-4-[(2,6-difluorophenyl)methyl]-1,2,4-triazol-3-one (15 mg, 24.9% yield) was obtained as a white solid. MS (m/z): 469.1 [M+H]⁺. ¹H NMR (400 MHz, DMSO-d₆) δ 8.28 (d, J=2.9 Hz, 1H), 7.88-7.77 (m, 2H), 7.52-7.45 (m, 1H), 7.20-7.07 (m, 4H), 4.97 (s, 2H), 3.34-3.20 (m, 1H), 2.10-1.88 (m, 4H), 1.80-1.56 (m, 4H).

| Compound No. | | | Appearance | | MS(m/z) |
|---|---|---|---|---|---|
| Method | Activity | Structure and Name | and Yield | ¹H NMR Data | [M + H]⁺ |
| Example 558 Method 102 | ++++ | 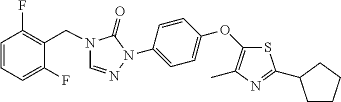 [SMILES: Cc1nc(C2CCCC2)sc1Oc1ccc(-n2ncn(Cc3c(F)cccc3F)c2=O)cc1] | white solid, yield: 24.9% | ¹H NMR (400 MHZ, DMSO-d₆) δ 8.28 (d, J = 2.9 Hz, 1H), 7.88-7.77 (m, 2H), 7.52-7.45 (m, 1H), 7.20-7.07 (m, 4H), 4.97 (s, 2H), 3.34-3.20 (m, 1H), 2.10-1.88 (m, 4H), 1.80-1.56 (m, 4H). | 469.1 |
| | | 2-(4-((2-cyclopentyl-4-methylthiazol- | | | |
| | | 5-yl)oxy)phenyl)-4-(2,6-difluorobenzyl)- | | | |
| | | 2,4-dihydro-3H-1,2,4-triazol-3-one | | | |

### Example 559: Method 103

### 5-(4-(4-(2,6-difluorobenzyl)-5-oxo-4,5-dihydro-1H-1,2,4-triazol-1-yl)-2-fluorophenoxy)-4-methylthiazole-2-carboxamide

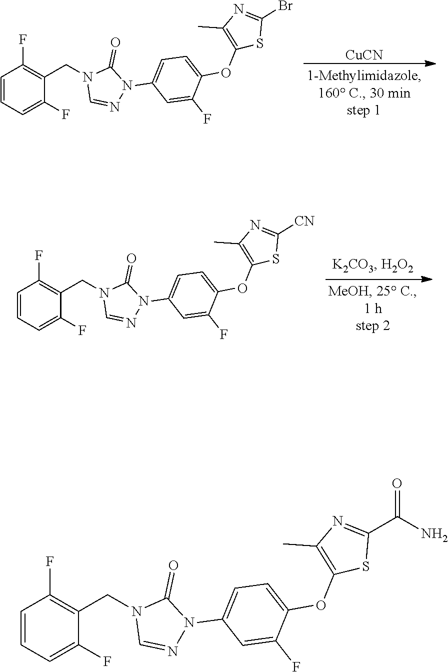 [SMILES: Cc1nc(Br)sc1Oc1ccc(-n2ncn(Cc3c(F)cccc3F)c2=O)cc1F ; "CuCN" ; Cc1nc(C#N)sc1Oc1ccc(-n2ncn(Cc3c(F)cccc3F)c2=O)cc1F ; Cc1nc(C(N)=O)sc1Oc1ccc(-n2ncn(Cc3c(F)cccc3F)c2=O)cc1F]

### Step 2: 5-(4-(4-(2,6-difluorobenzyl)-5-oxo-4,5-dihydro-1H-1,2,4-triazol-1-yl)-2-fluorophenoxy)-4-methylthiazole-2-carboxamide

To a solution of 5-[4-[4-[(2,6-difluorophenyl)methyl]-5-oxo-1,2,4-triazol-1-yl]-2-fluoro-phenoxy]-4-methyl-thiazole-2-carbonitrile (421 mg, 0.949 mmol) in Methanol (15 mL) were added potassium carbonate (656 mg, 4.75 mmol) and Hydrogen peroxide (6.0 mL, 300%). The reaction mixture was stirred at 25° C. for 1 h. The target was detected by LCMS. The reaction was taken up in EtOAc (200 mL) and the organics were washed with 2×100 mL water and then 1×100 mL saturated brine solution. The organics were then separated and dried (MgSO₄) before concentration to dryness. The crude was then purified by flash column chromatography eluted with 10-80% EtOAc in PE. The desired fractions were concentrated to dryness in vacuo. 5-[4-[4-[(2,6-difluorophenyl)methyl]-5-oxo-1,2,4-triazol-1-yl]-2-fluoro-phenoxy]-4-methyl-thiazole-2-carboxamide (164 mg, 37.400 yield) was obtained as a white solid. MS (m/z): 462.1 [M+H]⁺. ¹H NMR (400 MHz, DMSO-d₆) δ 8.35 (s, 1H), 8.09 (s, 1H), 7.90 (dd, J=12.6, 2.5 Hz, 1H), 7.79 (s, 1H), 7.71 (ddd, J=9.0, 2.7, 1.5 Hz, 1H), 7.48 (tt, J=8.5, 6.6 Hz, 1H), 7.38 (t, J=9.1 Hz, 1H), 7.26-7.08 (m, 2H), 4.98 (s, 2H), 2.30 (s, 3H).

The synthesis procedure of Examples (Compounds) 560-576 was similar to the procedure of Example 559.

| Compound | | | | | |
|---|---|---|---|---|---|
| No. | | | Appearance | | MS(m/z) |
| Method | Activity | Structure and Name | and Yield | ¹H NMR Data | [M + H]⁺ |
| Example 559 Method 103 | ++++ | 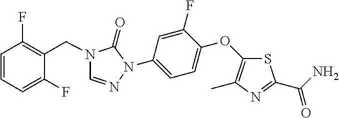 [SMILES: Cc1nc(C(N)=O)sc1Oc1ccc(-n2ncn(Cc3c(F)cccc3F)c2=O)cc1F] | white solid, yield: 37.4% | ¹H NMR (400 MHZ, DMSO-d₆) δ 8.35 (s, 1H), 8.09 (s, 1H), 7.90 (dd, J = 12.6, 2.5 Hz, 1H), 7.79 (s, 1H), 7.71 (ddd, J = 9.0, 2.7, 1.5 Hz, 1H), 7.48 (tt, J = 8.5, 6.6 Hz, 1H), 7.38 (t, J = 9.1 Hz, 1H), 7.26-7.08 (m, 2H), 4.98 (s, 2H), 2.30 (s, 3H). | 462.1 |
| | | 5-(4-(4-(2,6-difluorobenzyl)-5-oxo- | | | |
| | | 4,5-dihydro-1H-1,2,4-triazol-1- | | | |
| | | yl)-2-fluorophenoxy)-4-methylthiazole- | | | |
| | | 2-carboxamide | | | |
| Example 560 Method 103 | ++++ | 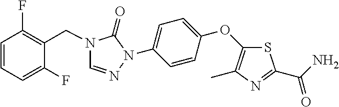 [SMILES: Cc1nc(C(N)=O)sc1Oc1ccc(-n2ncn(Cc3c(F)cccc3F)c2=O)cc1] | white solid, yield: 70.4% | ¹H NMR (400 MHZ, DMSO-d₆) δ 8.30 (s, 1H), 8.09 (s, 1H), 7.95-7.84 (m, 2H), 7.79 (s, 1H), 7.48 (tt, J = 8.5, 6.6 Hz, 1H), 7.31-7.21 (m, 2H), 7.21-7.08 (m, 2H), 4.98 (s, 2H), 2.26 (s, 3H). | 444.1 |
| | | 5-(4-(4-(2,6-difluorobenzyl)-5-oxo- | | | |
| | | 4,5-dihydro-1H-1,2,4-triazol-1- | | | |
| | | yl)phenoxy)-4-methylthiazole-2- | | | |
| | | carboxamide | | | |
| Example 561 Method 103 | ++++ | 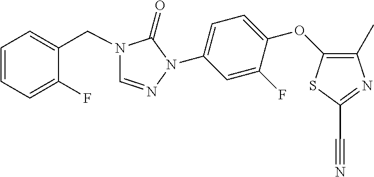 [SMILES: Cc1nc(C#N)sc1Oc1ccc(-n2ncn(Cc3ccccc3F)c2=O)cc1F] | Off-white solid, yield: 77.6 | ¹H NMR (400 MHZ, DMSO-d₆) δ 8.39 (s, 1H), 7.96 (dd, J = 12.5, 2.5 Hz, 1H), 7.77 (ddd, J = 9.1, 2.5, 1.4 Hz, 1H), 7.52 (t, J = 9.1 Hz, 1H), 7.47-7.35 (m, 2H), 7.31-7.16 (m, 2H), 4.97 (s, 2H), 2.36 (s, 3H). | 426.1 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-4-methylthiazole- | | | |
| | | 2-carbonitrile | | | |
| Example 562 Method 103 | ++++ | 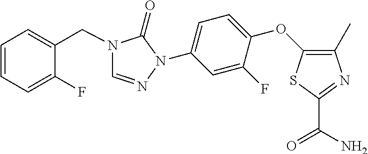 [SMILES: Cc1nc(C(N)=O)sc1Oc1ccc(-n2ncn(Cc3ccccc3F)c2=O)cc1F] | white solid, yield: 38.4% | ¹H NMR (400 MHZ, DMSO-d₆) δ 8.37 (s, 1H), 8.09 (s, 1H), 7.93 (dd, J = 12.6, 2.5 Hz, 1H), 7.80 (s, 1H), 7.74 (ddd, J = 9.1, 2.5, 1.4 Hz, 1H), 7.49-7.36 (m, 3H), 7.32-7.16 (m, 2H), 4.96 (s, 2H), 2.31 (s, 3H). | 444.1 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-4-methylthiazole- | | | |
| | | 2-carboxamide | | | |
| Example 563 Method 103 | ++++ | 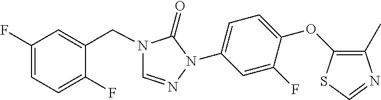 [SMILES: Cc1ncsc1Oc1ccc(-n2ncn(Cc3cc(F)ccc3F)c2=O)cc1F] | light-yellow solid, yield: 21.9% | ¹H NMR (400 MHZ, DMSO-d₆) δ 8.73 (s, 1H), 8.36 (s, 1H), 7.90 (dd, J = 12.6, 2.5 Hz, 1H), 7.71 (ddd, J = 9.1, 2.6, 1.5 Hz, 1H), 7.42-7.12 (m, 4H), 4.95 (s, 2H), 2.26 (s, 3H). 8.38 (d, J = 2.7 Hz, 1H), 8.10 (s, 3H). | 419.1 |
| | | 4-(2,5-difluorobenzyl)-2-(3-fluoro- | | | |
| | | 4-((4-methylthiazol-5-yl)oxy)phenyl)- | | | |
| | | 2,4-dihydro-3H-1,2,4-triazol-3-one | | | |
| Example 564 Method 103 | ++++ | 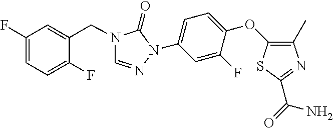 [SMILES: Cc1nc(C(N)=O)sc1Oc1ccc(-n2ncn(Cc3cc(F)ccc3F)c2=O)cc1F] | white solid, yield: 55.2% | ¹H NMR (400 MHZ, DMSO-d₆) δ 1H), 7.93 (dd, J = 12.6, 2.5 Hz, 1H), 7.88-7.67 (m, 2H), 7.56-7.36 (m, 1H), 7.29 (dddd, J = 16.9, 8.7, 7.2, 4.2 Hz, 2H), 7.14 (dt, J = 17.2, 7.9 Hz, 1H), 4.95 (s, 2H), 2.31 (s, 3H). 8.51 (d, J = 5.4 Hz, 1H), 8.38 (s, 3H). | 462.1 |
| | | 5-(4-(4-(2,5-difluorobenzyl)-5-oxo- | | | |
| | | 4,5-dihydro-1H-1,2,4-triazol-1- | | | |
| | | yl)-2-fluorophenoxy)-4-methylthiazole- | | | |
| | | 2-carboxamide | | | |
| Example 565 Method 103 | +++ | 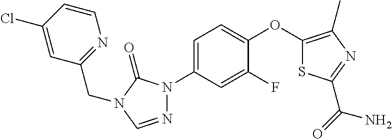 [SMILES: Cc1nc(C(N)=O)sc1Oc1ccc(-n2ncn(Cc3cc(Cl)ccn3)c2=O)cc1F] | white solid, yield: 12.5% | ¹H NMR (400 MHZ, DMSO-d₆) δ 1H), 8.10 (s, 1H), 7.94 (dd, J = 12.6, 2.5 Hz, 1H), 7.86-7.70 (m, 2H), 7.64 (d, J = 2.0 Hz, 1H), 7.50 (dd, J = 5.4, 2.0 Hz, 1H), 7.40 (t, J = 9.1 Hz, 1H), 5.05 (s, 2H), 2.31 (s, 3H). | 461.1 |
| | | 5-(4-(4-((4-chloropyridin-2-yl)me- | | | |
| | | 5-oxo-4,5-dihydro-1H-1,2,4- | | | |
| | | triazol-1-yl)-2-fluorophenoxy)-4- | | | |
| | | methylthiazole-2-carboxamide | | | |
| Example 566 Method 103 | ++++ | 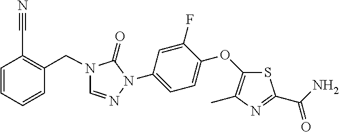 [SMILES: Cc1nc(C(N)=O)sc1Oc1ccc(-n2ncn(Cc3ccccc3C#N)c2=O)cc1F] | Yellow solid, yield: 62.6% | ¹H NMR (400 MHZ, DMSO-d₆) δ 8.39 (s, 1H), 8.09 (s, 1H), 7.97- 7.87 (m, 2H), 7.83-7.68 (m, 3H), 7.59-7.51 (m, 1H), 7.47 (d, J = 8.0 Hz, 1H), 7.40 (t, J = 9.2 Hz, 1H), 5.12 (s, 2H), 2.31 (s, 3H). | 451.0 |
| | | 5-(4-(4-(2-cyanobenzyl)-5-oxo-4,5- | | | |
| | | dihydro-1H-1,2,4-triazol-1-yl)- | | | |
| | | 2-fluorophenoxy)-4-methylthiazole- | | | |
| | | 2-carboxamide | | | |
| Example 567 Method 103 | ++++ | 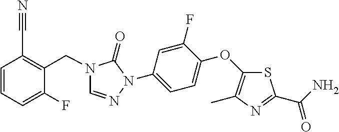 [SMILES: Cc1nc(C(N)=O)sc1Oc1ccc(-n2ncn(Cc3c(F)cccc3C#N)c2=O)cc1F] | Yellow solid, yield: 57.9% | ¹H NMR (400 MHZ, DMSO-d₆) δ 8.40 (d, J = 1.2 Hz, 1H), 8.09 (s, 1H), 7.89 (dd, J = 12.8, 2.4 Hz, 1H), 7.81-7.74 (m, 2H), 7.73-7.60 (m, 3H), 7.37 (t, J = 9.2 Hz, 1H), 5.12 (s, 2H), 2.30 (s, 3H). | 469.0 |
| | | 5-(4-(4-(2-cyano-6-fluorobenzyl)- | | | |
| | | 5-oxo-4,5-dihydro-1H-1,2,4-triazol- | | | |
| | | 1-yl)-2-fluorophenoxy)-4-methylthiazole- | | | |
| | | 2-carboxamide | | | |
| Example 568 Method 103 | ++++ | 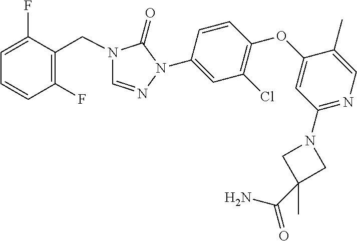 [SMILES: Cc1cnc(N2CC(C)(C(N)=O)C2)cc1Oc1ccc(-n2ncn(Cc3c(F)cccc3F)c2=O)cc1Cl] | White solid, yield: 38.5% | ¹H NMR (400 MHZ, Chloroform-d) δ 8.20 (d, J = 2.4 Hz, 1H), 7.97 (dd, J = 8.8, 2.4 Hz, 1H), 7.91 (s, 1H), 7.62 (s, 1H), 7.38 (tt, J = 8.4, 6.4 Hz, 1H), 7.15 (d, J = 8.8 Hz, 1H), 7.05- 6.94 (m, 2H), 6.13 (brs, 1H), 5.49- 5.33 (m, 2H), 4.99 (d, J = 1.2 Hz, 2H), 4.16 (d, J = 8.0 Hz, 2H), 3.73 (d, J = 8.0 Hz, 2H), 2.22 (s, 3H), 1.53 (s, 3H). | 541.2 |
| | | 1-(4-(2-chloro-4-(4-(2,6-difluorobenzyl)- | | | |
| | | 5-oxo-4,5-dihydro-1H-1,2,4-triazol- | | | |
| | | 1-yl)phenoxy)-5-methylpyridin-2-yl)- | | | |
| | | 3-methylazetidine-3-carboxamide | | | |
| Example 569 Method 103 | ++++ | 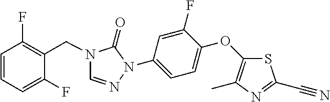 [SMILES: Cc1nc(C#N)sc1Oc1ccc(-n2ncn(Cc3c(F)cccc3F)c2=O)cc1F] | yellow solid, yield: 35.5% | ¹H NMR (400 MHZ, DMSO-d₆) δ 8.38 (s, 1H), 7.93 (dd, J = 12.4, 2.4 Hz, 1H), 7.74 (ddd, J = 9.2, 2.6, 1.2 Hz, 1H), 7.55-7.45 (m, 2H), 7.16 (t, J = 8.0 Hz, 2H), 4.98 (s, 2H), 2.35 (s, 3H). | 444.1 |
| | | 5-(4-(4-(2,6-difluorobenzyl)-5- | | | |
| | | oxo-4,5-dihydro-1H-1,2,4-triazol- | | | |
| | | 1-yl)-2-fluorophenoxy)-4-methylthiazole- | | | |
| | | 2-carbonitrile | | | |
| Example 570 Method 103 | +++ | 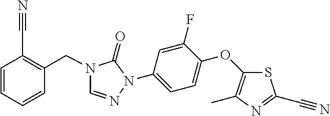 [SMILES: Cc1nc(C#N)sc1Oc1ccc(-n2ncn(Cc3ccccc3C#N)c2=O)cc1F] | Yellow solid, yield: 26.6% | ¹H NMR (400 MHZ, DMSO-d₆) δ 8.40 (s, 1H), 8.01-7.87 (m, 2H), 7.85-7.65 (m, 2H), 7.59-7.45 (m, 3H), 5.12 (s, 2H), 2.36 (s, 3H). | 433.0 |
| | | 5-(4-(4-(2-cyanobenzyl)-5-oxo-4,5- | | | |
| | | dihydro-1H-1,2,4-triazol-1-yl)-2- | | | |
| | | fluorophenoxy)-4-methylthiazole-2-carbonitrile | | | |
| Example 571 Method 103 | ++++ | 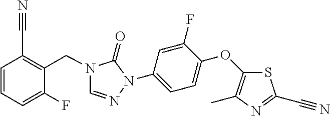 [SMILES: Cc1nc(C#N)sc1Oc1ccc(-n2ncn(Cc3c(F)cccc3C#N)c2=O)cc1F] | Yellow solid, yield: 24.1% | ¹H NMR (400 MHZ, DMSO-d₆) δ 8.42 (d, J = 1.2 Hz, 1H), 7.92 (dd, J = 12.4, 2.4 Hz, 1H), 7.84-7.71 (m, 2H), 7.71-7.60 (m, 2H), 7.51 (t, J = 9.2 Hz, 1H), 5.13 (d, J = 1.2 Hz, 2H), 2.36 (d, J = 4.8 Hz, 3H). | 451.0 |
| | | 5-(4-(4-(2-cyano-6-fluorobenzyl)-5- | | | |
| | | oxo-4,5-dihydro-1H-1,2,4-triazol- | | | |
| | | 1-yl)-2-fluorophenoxy)-4-methylthiazole- | | | |
| | | 2-carbonitrile | | | |
| Example 572 Method 103 | ++++ | 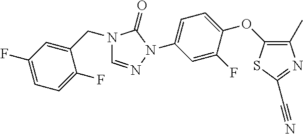 [SMILES: Cc1nc(C#N)sc1Oc1ccc(-n2ncn(Cc3cc(F)ccc3F)c2=O)cc1F] | Light-yellow solid, yield: 72.3% | ¹H NMR (400 MHZ, DMSO-d₆) δ 8.39 (s, 1H), 7.95 (dd, J = 12.5, 2.5 Hz, 1H), 7.77 (ddd, J = 9.1, 2.6, 1.4 Hz, 1H), 7.53 (t, J = 9.1 Hz, 1H), 7.40-7.14 (m, 3H), 4.95 (s, 2H), 2.36 (s, 3H). | 444.1 |
| | | 5-(4-(4-(2,5-difluorobenzyl)-5-oxo- | | | |
| | | 4,5-dihydro-1H-1,2,4-triazol- | | | |
| | | 1-yl)-2-fluorophenoxy)-4-methylthiazole- | | | |
| | | 2-carbonitrile | | | |
| Example 573 Method 103 | +++ | 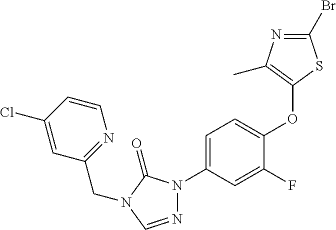 [SMILES: Cc1nc(Br)sc1Oc1ccc(-n2ncn(Cc3cc(Cl)ccn3)c2=O)cc1F] | light-yellow solid, yield: 65.4% | ¹H NMR (400 MHZ, DMSO-d₆) δ 8.51 (d, J = 5.4 Hz, 1H), 8.37 (s, 1H), 7.91 (dd, J = 12.6, 2.5 Hz, 1H), 7.73 (ddd, J = 9.1, 2.6, 1.5 Hz, 1H), 7.69-7.59 (m, 1H), 7.50 (dd, J = 5.4, 2.0 Hz, 1H), 7.36 (t, J = 9.1 Hz, 1H), 5.05 (s, 2H), 2.23 (s, 3H). | 496.1, 498.1 |
| | | 2-(4-((2-bromo-4-methylthiazol- | | | |
| | | 5-yl)oxy)-3-fluorophenyl)-4-((4- | | | |
| | | chloropyridin-2-yl)methyl)-2,4- | | | |
| | | dihydro-3H-1,2,4-triazol-3-one | | | |
| Example 574 Method 103 | +++ | 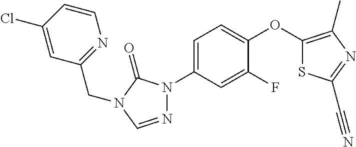 [SMILES: Cc1nc(C#N)sc1Oc1ccc(-n2ncn(Cc3cc(Cl)ccn3)c2=O)cc1F] | Off-white solid, yield: 70.7% | ¹H NMR (400 MHZ, DMSO-d₆) δ 8.51 (d, J = 5.4 Hz, 1H), 8.40 (s, 1H), 7.97 (dd, J = 12.5, 2.5 Hz, 1H), 7.79 (ddd, J = 9.1, 2.6, 1.4 Hz, 1H), 7.64 (d, J = 2.0 Hz, 1H), 7.57-7.45 (m, 2H), 5.05 (s, 2H), 2.36 (s, 3H). | 443.1 |
| | | 5-(4-(4-((4-chloropyridin-2-yl)methyl)- | | | |
| | | 5-oxo-4,5-dihydro-1H-1,2, | | | |
| | | 4-triazol-1-yl)-2-fluorophenoxy)- | | | |
| | | 4-methylthiazole-2-carbonitrile | | | |
| Example 575 Method 103 | ++ | 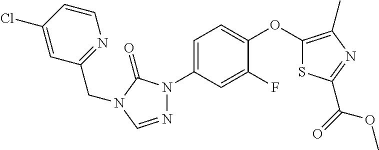 [SMILES: COC(=O)c1nc(C)c(Oc2ccc(-n3ncn(Cc4cc(Cl)ccn4)c3=O)cc2F)s1] | white solid, yield: 28.4% | ¹H NMR (400 MHZ, DMSO-d₆) δ 8.51 (d, J = 5.4 Hz, 1H), 8.40 (s, 1H), 7.96 (dd, J = 12.5, 2.5 Hz, 1H), 7.78 (ddd, J = 9.1, 2.5, 1.4 Hz, 1H), 7.64 (d, J = 2.0 Hz, 1H), 7.54-7.42 (m, 2H), 5.05 (s, 2H), 3.87 (s, 3H), 2.35 (s, 3H). | 476.1 |
| | | methyl | | | |
| | | 5-(4-(4-((4-chloropyridin-2-yl)methyl)- | | | |
| | | 5-oxo-4,5-dihydro-1H-1,2,4- | | | |
| | | triazol-1-yl)-2-fluorophenoxy)-4- | | | |
| | | methylthiazole-2-carboxylate | | | |
| Example 576 Method 103 | ++++ | 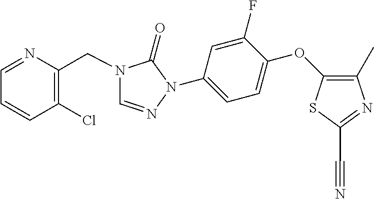 [SMILES: Cc1nc(C#N)sc1Oc1ccc(-n2ncn(Cc3ncccc3Cl)c2=O)cc1F] | white solid, yield: 17.3% | ¹H NMR (400 MHZ, Chloroform-d) δ 8.49-8.43 (m, 1H), 8.02 (dd, J = 12.4, 2.4 Hz, 1H), 7.92-7.84 (m, 1H), 7.80-7.71 (m, 2H), 7.29- 7.24 (m, 1H), 7.17 (t, J = 8.8 Hz, 1H), 5.17 (s, 2H), 2.44 (s, 3H). | 443.2 |
| | | 5-(4-(4-((3-chloropyridin-2-yl)methyl)- | | | |
| | | 5-oxo-4,5-dihydro-1H-1,2, | | | |
| | | 4-triazol-1-yl)-2-fluorophenoxy)- | | | |
| | | 4-methylthiazole-2-carbonitrile | | | |

### Example 577: Method 104

### 4-(2,6-difluorobenzyl)-2-(4-((2-(3-hydroxypentan-3-yl)-4-methylthiazol-5-yl)oxy)phenyl)-2,4-dihydro-3H-1,2,4-triazol-3-one

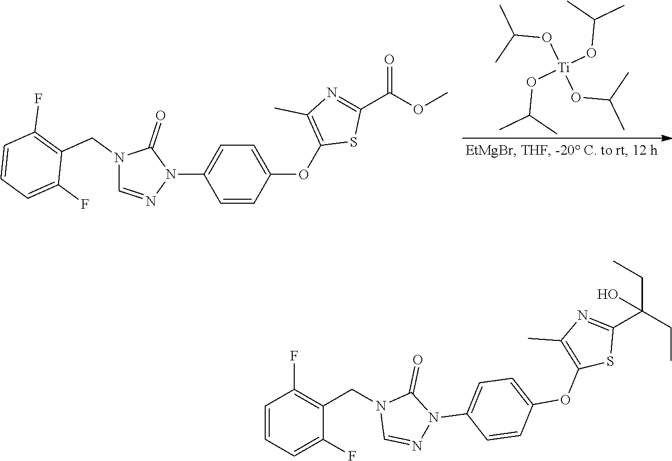

To a solution of methyl 5-(4-(4-(2,6-difluorobenzyl)-5-oxo-4,5-dihydro-1H-1,2,4-triazol-1-yl)phenoxy)-4-methylthiazole-2-carboxylate (150 mg, 0.317 mmol) in TIFF (20 mL) was added titanium(IV) transferrin (126 mg, 0.444 mmol) at −20° C. under Ar.

Then bromo(ethyl)magnesium (118 mg, 0.889 mmol) was added slowly. The resulted mixture was left to warm to rt and stirred 12 h at rt. The target was detected by LCMS. The reaction was taken up in EtOAc (100 mL) and the organics were washed with 2×100 mL water and then 1×100 m saturated brine solution. The organics were then separated and dried (MgSO₄) before concentration to dryness. The crude was then purified by pre-HPLC. 4-(2,6-difluorobenzyl)-2-(4-((2-(3-hydroxypentan-3-yl)-4-methylthiazol-5-yl)oxy)phenyl)-2,4-dihydro-3H-1,2,4-triazol-3-one (22 mg, 14.200 yield) was obtained as a yellow solid was obtained as a yellow solid. MS (m/z): 487.1 [M+H]⁺. ¹H NMR (400 MHz, DMSO) δ 8.28 (s, 1H), 7.90-7.77 (m, 2H), 7.48 (tt, J 8.4, 6.6 Hz, 1H), 7.16 (t, J=8.0 Hz, 2H), 7.12-7.03 (m, 2H), 5.47 (brs, 1H), 4.98 (s, 2H), 2.14 (s, 3H), 1.78 (ddq, J=39.1, 14.3, 7.4 Hz, 4H), 0.79 (t, J=7.4 Hz, 6H).

The synthesis procedure of Examples (Compounds) 578-584 was similar to the procedure of Example 577.

| | | | Appear- | | |
|---|---|---|---|---|---|
| Compound | | | ance | | MS |
| No. | | | and | | (m/z) |
| Method | Activity | Structure and Name | Yield | ¹H NMR Data | [M + H]⁺ |
| Example 577 Method 104 | ++++ | 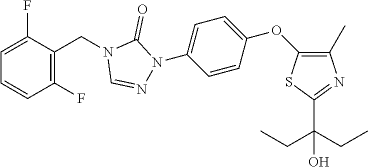 [SMILES: CCC(O)(CC)c1nc(C)c(Oc2ccc(-n3ncn(Cc4c(F)cccc4F)c3=O)cc2)s1] | yellow solid, yield: 14.2% | ¹H NMR (400 MHz, DMSO-d₆) δ 8.28 (s, 1H), 7.90-7.77 (m, 2H), 7.48 (tt, J = 8.4, 6.6 Hz, 1H), 7.16 (t, J = 8.0 Hz, 2H), 7.12-7.03 (m, 2H), 5.47 (brs, 1H), 4.98 (s, 2H), 2.14 (s, 3H), 1.78 (ddq, J = 39.1, 14.3, 7.4 Hz, 4H), 0.79 (t, J = 7.4 Hz, 6H). | 487.1 |
| | | 4-(2,6-difluorobenzyl)-2-(4-((2-(3- | | | |
| | | hydroxypentan-3-yl)-4-methylthiazol- | | | |
| | | 5-yl)oxy)phenyl)-2,4-dihydro- | | | |
| | | 3H-1,2,4-triazol-3-one | | | |
| Example 578 Method 104 | +++ | 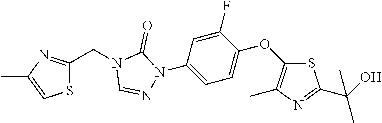 [SMILES: Cc1csc(Cn2cnn(-c3ccc(Oc4sc(C(C)(C)O)nc4C)c(F)c3)c2=O)n1] | white solid, yield: 14.1% | NA | 462.1 |
| | | 2-(3-fluoro-4-((2-(2-hydroxypropan- | | | |
| | | 2-yl)-4-methylthiazol-5-yl)oxy) | | | |
| | | phenyl)-4-((4-methylthiazol-2-yl) | | | |
| | | methyl)-2,4-dihydro-3H-1,2,4- | | | |
| | | triazole-3-one | | | |
| Example 579 Method 104 | ++++ | 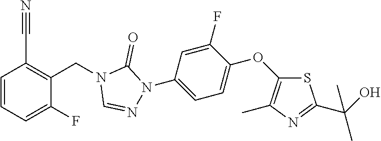 [SMILES: Cc1nc(C(C)(C)O)sc1Oc1ccc(-n2ncn(Cc3c(F)cccc3C#N)c2=O)cc1F] | white solid, yield: 16.3% | ¹H NMR (400 MHz, DMSO-d₆) δ 8.39 (d, J = 1.2 Hz, 1H), 7.85 (dd, J = 12.8, 2.4 Hz, 1H), 7.77 (dd, J = 6.8, 2.0 Hz, 1H), 7.70-7.58 (m, 3H), 7.20 (t, J = 9.2 Hz, 1H), 5.94 (s, 1H), 5.12 (s, 2H), 2.18 (s, 3H), 1.46 (s, 6H). | 484.1 |
| | | 3-fluoro-2-((1-(3-fluoro-4-((2-(2- | | | |
| | | hydroxypropan-2-yl)-4-methylthiazol- | | | |
| | | 5-yl)oxy)phenyl)-5-oxo-1,5- | | | |
| | | dihydro-4H-1,2,4-triazol-4-yl) | | | |
| | | methyl)benzonitrile | | | |
| Example 580 Method 104 | ++++ | 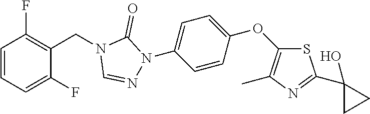 [SMILES: Cc1nc(C2(O)CC2)sc1Oc1ccc(-n2ncn(Cc3c(F)cccc3F)c2=O)cc1] | yellow solid, yield: 17.9% | ¹H NMR (400 MHz, DMSO-d₆) δ 8.32 (s, 1H), 7.98-7.86 (m, 2H), 7.49 (tt, J = 8.4, 6.6 Hz, 1H), 7.37-7.23 (m, 2H), 7.23-7.07 (m, 2H), 4.99 (s, 2H), 3.05 (q, J = 7.3 Hz, 2H), 2.32 (s, 3H), 1.10 (t, J = 7.3 Hz, 2H). | 457.1 |
| | | 4-(2,6-difluorobenzyl)-2-(4-((2-(1- | | | |
| | | hydroxycyclopropyl)-4-methylthiazol- | | | |
| | | 5-yl)oxy)phenyl)-2,4-dihydro- | | | |
| | | 3H-1,2,4-triazol-3-one | | | |
| Example 581 Method 104 | ++++ | 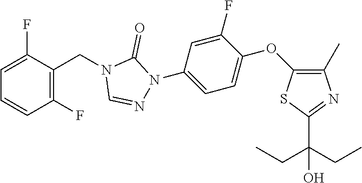 [SMILES: CCC(O)(CC)c1nc(C)c(Oc2ccc(-n3ncn(Cc4c(F)cccc4F)c3=O)cc2F)s1] | yelow solid, yield: 25.3% | ¹H NMR (400 MHz, DMSO-d₆) δ 8.34 (s, 1H), 7.87 (dd, J = 12.6, 2.5 Hz, 1H), 7.68 (ddd, J = 9.1, 2.6, 1.5 Hz, 1H), 7.49 (tt, J = 8.5, 6.6 Hz, 1H), 7.16 (t, J = 8.2 Hz, 3H), 5.48 (s, 1H), 4.98 (s, 2H), 2.18 (s, 3H), 1.92-1.65 (m, 4H), 0.78 (t, J = 7.4 Hz, 6H). | 505.1 |
| | | 4-(2,6-difluorobenzyl)-2-(3-fluoro- | | | |
| | | 4-((2-(3-hydroxypentan-3-yl)-4- | | | |
| | | methylthiazol-5-yl)oxy)phenyl)- | | | |
| | | 2,4-dihydro-3H-1,2,4-triazol-3-one | | | |
| Example 582 Method 104 | ++++ | 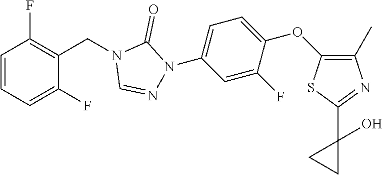 [SMILES: Cc1nc(C2(O)CC2)sc1Oc1ccc(-n2ncn(Cc3c(F)cccc3F)c2=O)cc1F] | yellow solid, yield: 22.7% | ¹H NMR (400 MHz, DMSO-d₆) δ 8.37 (s, 1H), 7.92 (dd, J = 12.6, 2.5 Hz, 1H), 7.74 (ddd, J = 9.1, 2.6, 1.5 Hz, 1H), 7.48 (dtd, J = 11.8, 8.8, 5.9 Hz, 2H), 7.22-7.11 (m, 2H), 4.99 (s, 2H), 3.04 (q, J = 7.3 Hz, 2H), 2.36 (s, 3H), 1.09 (t, J = 7.3 Hz, 2H). | 475.1 |
| | | 4-(2,6-difluorobenzyl)-2-(3-fluoro- | | | |
| | | 4-((2-(1-hydroxycyclopropyl)-4- | | | |
| | | methylthiazol-5-yl)oxy)phenyl)- | | | |
| | | 2,4-dihydro-3H-1,2,4-triazol-3-one | | | |
| Example 583 Method 104 | ++++ | 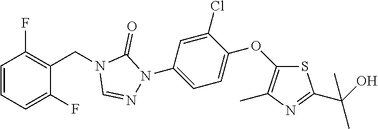 [SMILES: Cc1nc(C(C)(C)O)sc1Oc1ccc(-n2ncn(Cc3c(F)cccc3F)c2=O)cc1Cl] | white solid, yield: 12.8% | ¹H NMR (400 MHz, DMSO-d₆) δ 8.34 (s, 1H), 8.07 (d, J = 2.8 Hz, 1H), 7.80 (dd, J = 9.2, 2.4 Hz, 1H), 7.48 (tt, J = 8.4, 6.8 Hz, 1H), 7.20- 7.10 (m, 2H), 5.94 (s, 1H), 5.12 (s, 2H), 2.19 (s, 3H), 1.47 (s, 6H). | 493.8 |
| | | 2-(3-chloro-4-((2-(2-hydroxypropan- | | | |
| | | 2-yl)-4-methylthiazol-5-yl)oxy) | | | |
| | | phenyl)-4-(2,6-difluorobenzyl)- | | | |
| | | 2,4-dihydro-3H-1,2,4-triazol-3-one | | | |
| Example 584 Method 104 | +++ | 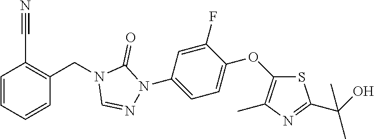 [SMILES: Cc1nc(C(C)(C)O)sc1Oc1ccc(-n2ncn(Cc3ccccc3C#N)c2=O)cc1F] | Yellow solid, yield: 14.7% | ¹H NMR (400 MHz, DMSO-d₆) δ 8.37 (s, 1H), 7.94-7.86 (m, 2H), 7.76-7.68 (m, 2H), 7.59-7.51 (m, 1H), 7.47 (d, J = 8.0 Hz, 1H), 7.23 (t, J = 9.2 Hz, 1H), 5.94 (s, 1H), 5.12 (s, 2H), 2.19 (s, 3H), 1.47 (s, 6H). | 466.1 |
| | | 2-((1-(3-fluoro-4-((2-(2-hydroxypropan-2- | | | |
| | | yl)-4-methylthiazol-5-yl) | | | |
| | | oxy)phenyl)-5-oxo-1,5-dihydro-4H- | | | |
| | | 1,2,4-triazol-4-yl)methyl)benzonitrile | | | |

### Example 585: Method 105

### 4-(2,6-difluorobenzyl)-2-(3-fluoro-4-((2-(3-hydroxyoxetan-3-yl)-4-methylthiazol-5-yl)oxy)phenyl)-2,4-dihydro-3H-1,2,4-triazol-3-one

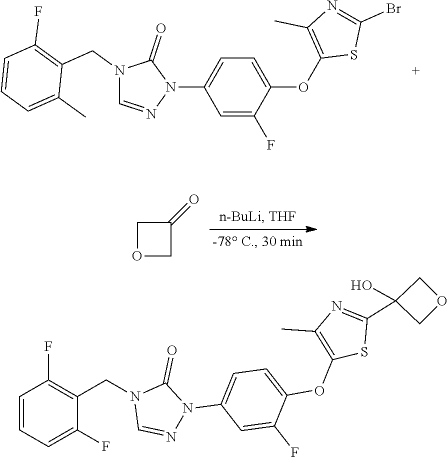 [SMILES: Cc1cccc(F)c1Cn1cnn(-c2ccc(Oc3sc(Br)nc3C)c(F)c2)c1=O ; O=C1COC1 ; Cc1nc(C2(O)COC2)sc1Oc1ccc(-n2ncn(Cc3c(F)cccc3F)c2=O)cc1F]

To a solution of 2-[4-(2-bromo-4-methyl-thiazol-5-yl)oxy-3-fluoro-phenyl]-4-[(2,6-difluorophenyl)methyl]-1,2,4-triazol-3-one (150 mg, 0.302 mmol) in TIFF (20 mL) was added butyllithium (23 mg, 0.362 mmol) at −78 TC under Ar. The reaction mixture was stirred at −78 TC for 5 min and oxetan-3-one (26 mg, 0.362 mmol) was added. The reaction mixture was stirred at −78 TC for 30 min. The reaction was taken up in EtOAc (100 mL) and the organics were washed with 2×50 mL water and then 1×50 mL saturated brine solution. The organics were then separated and dried (MgSO₄) before concentration to dryness. The crude was then purified by reversed-phase column. 4-[(2,6-difluorophenyl)methyl]-2-[3-fluoro-4-[2-(3-hydroxyoxetan-3-yl)-4-methyl-thiazol-5-yl]oxy-phenyl]-1,2,4-triazol-3-one (65 mg, 43.9 yield) was obtained as a white solid. MS (m/z): 491.1 [M+H]⁺. ¹H NMR (400 MHz, DMSO-d₆) δ 8.34 (s, 1H), 7.87 (dd, J=12.6, 2.6 Hz, 1H), 7.67 (ddd, J=9.1, 2.6, 1.4 Hz, 1H), 7.48 (tt, J=8.4, 6.6 Hz, 1H), 7.32-7.08 (m, 4H), 4.98 (s, 2H), 4.91-4.85 (m, 2H),4-3,4.71-4.63 (m, 2H), 2.26 (s, 3H).

The synthesis procedure of Example (Compound) 586 was similar to the procedure of Example 585.

| | | | | | MS |
|---|---|---|---|---|---|
| | | | | | (m/z) |
| Compound | | | Appearance | | [M + |
| No. Method | Activity | Structure and Name | and Yield | ¹H NMR Data | H]⁺ |
| Example 585 Method 105 | ++++ | 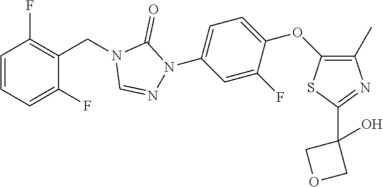 [SMILES: Cc1nc(C2(O)COC2)sc1Oc1ccc(-n2ncn(Cc3c(F)cccc3F)c2=O)cc1F] | white solid, yield: 43.9% | ¹H NMR (400 MHz, DMSO-d₆) δ 8.34 (s, 1H), 7.87 (dd, J = 12.6, 2.6 Hz, 1H), 7.67 (ddd, J = 9.1, 2.6, 1.4 Hz, 1H), 7.48 (tt, J = 8.4, 6.6 Hz, 1H), 7.32-7.08 (m, 4H), 4.98 (s, 2H), 4.91-4.85 (m, 2H), 4.71-4.63 (m, 2H), 2.26 (s, 3H). | 491.1 |
| | | 4-(2,6-difluorobenzyl)-2-(3-fluoro- | | | |
| | | 4-((2-(3-hydroxyoxetan-3-yl)- | | | |
| | | 4-methylthiazol-5-yl)oxy)phenyl)- | | | |
| | | 2,4-dihydro-3H-1,2,4-triazol-3-one | | | |
| Example 586 Method 105 | ++++ | 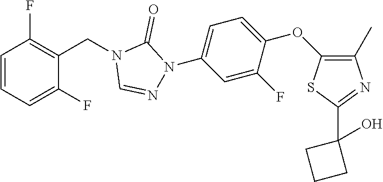 [SMILES: Cc1nc(C2(O)CCC2)sc1Oc1ccc(-n2ncn(Cc3c(F)cccc3F)c2=O)cc1F] | white solid, yield: 32.8% | ¹H NMR (400 MHz, DMSO-d₆) δ 8.34 (s, 1H), 7.87 (dd, J = 12.6, 2.5 Hz, 1H), 7.67 (ddd, J = 9.1, 2.6, 1.4 Hz, 1H), 7.48 (tt, J = 8.5, 6.7 Hz, 1H), 7.18 (dt, J = 16.5, 8.6 Hz, 3H), 6.46 (s, 1H), 4.98 (s, 2H), 2.56-2.46 (m, 2H), 2.28-2.18 (m, 2H), 2.21 (s, 3H), 1.92-1.76 (m, 2H). | 489.1 |
| | | 4-(2,6-difluorobenzyl)-2-(3-fluoro- | | | |
| | | 4-((2-(1-hydroxycyclobutyl)- | | | |
| | | 4-methylthiazol-5-yl)oxy)phenyl)- | | | |
| | | 2,4-dihydro-3H-1,2,4-triazol-3-one | | | |

### Example 587: Method 106

### 2-(4-((2-((2S,3S)-3-amino-2-methylazetidine-1-carbonyl)-4-methylthiazol-5-yl)oxy)-3-fluorophenyl)-4-(2-fluorobenzyl)-2,4-dihydro-3H-1,2,4-triazol-3-one

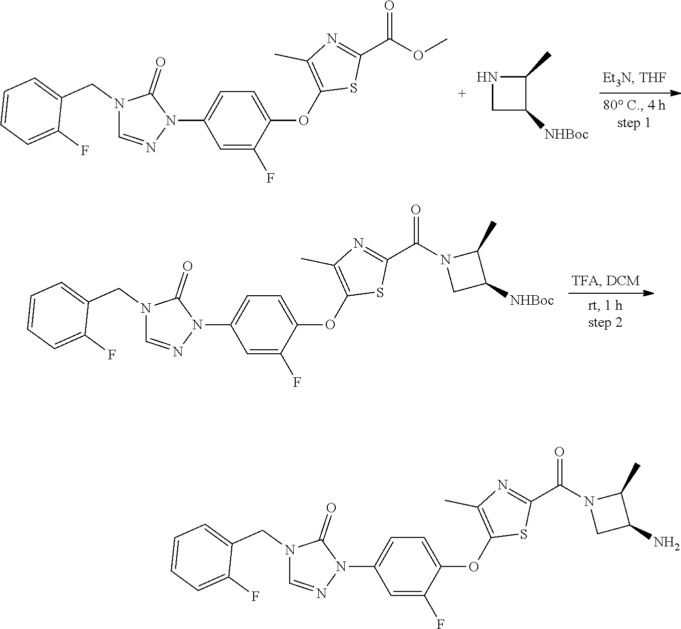 [SMILES: COC(=O)c1nc(C)c(Oc2ccc(-n3ncn(Cc4ccccc4F)c3=O)cc2F)s1 ; C[C@@H]1NC[C@@H]1NC(=O)OC(C)(C)C ; Cc1nc(C(=O)N2C[C@H](NC(=O)OC(C)(C)C)[C@@H]2C)sc1Oc1ccc(-n2ncn(Cc3ccccc3F)c2=O)cc1F ; Cc1nc(C(=O)N2C[C@H](N)[C@@H]2C)sc1Oc1ccc(-n2ncn(Cc3ccccc3F)c2=O)cc1F]

### Step 1: tert-butyl ((2S,3S)-1-(5-(2-fluoro-4-(4-(2-fluorobenzyl)-5-oxo-4,5-dihydro-1H-1,2,4-triazol-1-yl)phenoxy)-4-methylthiazole-2-carbonyl)-2-methylazetidin-3-yl)carbamate

To a solution of methyl 5-[2-fluoro-4-[4-[(2-fluorophenyl)methyl]-5-oxo-1,2,4-triazol-1-yl]phenoxy]-4-methyl-thiazole-2-carboxylate (60 mg, 0.131 mmol) in THF (5 mL) were added 2,2-dimethyl-N-[rac-(2S,3S)-2-methylazetidin-3-yl]propanamide (45 mg, 0.262 mmol) and Et₃N (2 mL). The reaction mixture was stirred at 80° C. for 4 h. The target was detected by LCMS. The reaction was taken up in EtOAc (100 mL) and the organics were washed with 2×50 mL water and then 1×50 mL saturated brine solution. The organics were then separated and dried (MgSO₄) before concentration to dryness. The crude was then purified by flash column chromatography eluted with 10-80% EtOAc in PE. Tert-butyl ((2S,3S)-1-(5-(2-fluoro-4-(4-(2-fluorobenzyl)-5-oxo-4,5-dihydro-1H-1,2,4-triazol-1-yl)phenoxy)-4-methylthiazole-2-carbonyl)-2- methylazetidin-3-yl)carbamate (53 mg, 66.1% yield) was obtained as a white solid. MS (m/z): 613.2 [M+H]⁺.

Step 2: To a solution of tert-butyl N-[(2S,3S)-1-[5-[2-fluoro-4-[4-[(2-fluorophenyl)methyl]-5-oxo-1,2,4-triazol-1-yl]phenoxy]-4-methyl-thiazole-2-carbonyl]-2-methyl-azetidin-3-yl]carba mate (50 mg, 0.0816 mmol) in DCM (3 mL) was added TFA (3 mL). The reaction mixture was stirred at 25° C. for 1 h. The crude was purified by reversed-phase column. 2-[4-[2-[(2S,3S)-3-amino-2-methyl-azetidine-1-carbonyl]-4-methyl-thiazol-5-yl]oxy-3-fluoro-phenyl]-4-[(2-fluorophenyl)methyl]-1,2,4-triazol-3-one (21 mg, 50.2%) yield was obtained as an off-white solid. MS (m/z): 613.2 [M+H]⁺. ¹H NMR (400 MHz, DMSO-d₆) δ 8.38 (s, 1H), 7.93 (dd, J=12.5, 2.5 Hz, 1H), 7.75 (d, J=9.1 Hz, 1H), 7.41 (qd, J=7.4, 2.9 Hz, 3H), 7.33-7.14 (m, 2H), 4.96 (s, 2H), 4.77 (dd, J=10.6, 7.1 Hz, 1H), 4.55 (dd, J=11.4, 4.6 Hz, 1H), 4.35-4.00 (m, 2H), 2.32 (s, 3H), 1.51 (d, J=6.7 Hz, 3H).

The synthesis procedure of Examples (Compounds) 588-611 was similar to the procedure of Example 587.

| Com- | | | Appear- | | MS |
|---|---|---|---|---|---|
| pound | | | ance | | (m/z) |
| No. | Activ- | | and | | [M + |
| Method | ity | Structure and Name | Yield | ¹H NMR Data | H]⁺ |
| Example 587 Method 106 | ++++ | 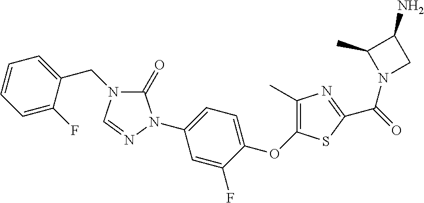 [SMILES: Cc1nc(C(=O)N2C[C@H](N)[C@@H]2C)sc1Oc1ccc(-n2ncn(Cc3ccccc3F)c2=O)cc1F] | white solid, yield: 50.2% | ¹H NMR (400 MHz, DMSO-d₆) δ 8.38 (s, 1H), 7.93 (dd, J = 12.5, 2.5 Hz, 1H), 7.75 (d, J = 9.1 Hz, 1H), 7.41 (qd, J = 7.4, 2.9 Hz, 3H), 7.33-7.14 (m, 2H), 4.96 (s, 2H), 4.77 (dd, J = 10.6, 7.1 Hz, 1H), 4.55 (dd, J = 11.4, 4.6 Hz, 1H), 4.35-4.00 (m, 2H), 2.32 (s, 3H), 1.51 (d, J = 6.7 Hz, 3H). | 613.2 |
| | | 2-(4-((2-((2S,3S)-3-amino-2-methylazetidine- | | | |
| | | 1-carbonyl)-4-methylthiazol- | | | |
| | | 5-yl)oxy)-3-fluorophenyl)- | | | |
| | | 4-(2-fluorobenzyl)-2,4-dihydro-3H- | | | |
| | | 1,2,4-triazol-3-one | | | |
| Example 588 Method 106 | ++++ | 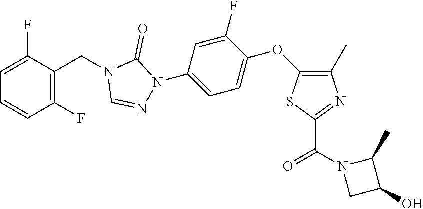 [SMILES: Cc1nc(C(=O)N2C[C@H](O)[C@@H]2C)sc1Oc1ccc(-n2ncn(Cc3c(F)cccc3F)c2=O)cc1F] | white solid, yield: 49.2% | ¹H NMR (400 MHz, DMSO-d₆) δ 8.35 (s, 1H), 7.89 (dd, J = 12.8, 2.4 Hz, 1H), 7.74-7.66 (m, 1H), 7.48 (tt, J = 8.4, 6.4 Hz, 1H), 7.37 (q, J = 9.2 Hz, 1H), 7.15 (t, J = 8.0 Hz, 2H), 5.61-5.50 (m, 1H), 4.97 (s, 2H), 4.78-4.69 (m, 1H), 4.65- 4.50 (m, 2H), 4.29-4.19 (m, 1H), 2.28 (d, J = 6.9 Hz, 3H), 1.48-1.28 (m, 3H). | 532.2 |
| | | 4-(2,6-difluorobenzyl)-2-(3-fluoro- | | | |
| | | 4-((2-((2S,3S)-3-hydroxy-2-methylazetidine- | | | |
| | | 1-carbonyl)-4-methylthiazol- | | | |
| | | yl)oxy)phenyl)-2,4-dihydro- | | | |
| | | 3H-1,2,4-triazol-3-one | | | |
| Example 589 Method 106 | ++++ | 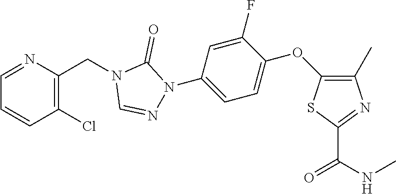 [SMILES: CNC(=O)c1nc(C)c(Oc2ccc(-n3ncn(Cc4ncccc4Cl)c3=O)cc2F)s1] | white solid, yield: 32.5% | ¹H NMR (400 MHz, Chloroform- d) δ 8.47 (dd, J = 4.4, 1.6 Hz, 1H), 7.97 (dd, J = 12.4, 2.4 Hz, 1H), 7.80 (dt, J = 9.2, 2.0 Hz, 1H), 7.77-7.71 (m, 2H), 7.29-7.23 (m, 1H), 7.13- 7.03 (m, 2H), 5.16 (s, 2H), 3.00 (d, J = 5.2 Hz, 3H), 2.34 (s, 3H). | 475.2 |
| | | 5-(4-(4-((3-chloropyridin-2-yl) | | | |
| | | methyl)-5-oxo-4,5-dihydro-1H-1,2,4- | | | |
| | | triazol-1-yl)-2-fluorophenoxy)-N,4- | | | |
| | | dimethylthiazole-2-carboxamide | | | |
| Example 590 Method 106 | ++++ | 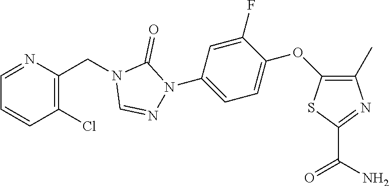 [SMILES: Cc1nc(C(N)=O)sc1Oc1ccc(-n2ncn(Cc3ncccc3Cl)c2=O)cc1F] | white solid, yield: 24.1% | ¹H NMR (400 MHz, Chloroform- d) δ 8.47 (dd, J = 4.8, 1.2 Hz, 1H), 7.98 (dd, J = 12.4, 2.4 Hz, 1H), 7.81 (dt, J = 9.2, 2.0 Hz, 1H), 7.78-7.72 (m, 2H), 7.29-7.23 (m, 1H), 7.11 (t, J = 8.8 Hz, 1H), 6.99 (brs, 1H), 5.44 (brs, 1H), 5.17 (s, 2H), 2.36 (s, 3H). | 461.3 |
| | | 5-(4-(4-((3-chloropyridin-2-yl)methyl)- | | | |
| | | 5-oxo-4,5-dihydro-1H-1,2,4- | | | |
| | | triazol-1-yl)-2-fluorophenoxy)-4- | | | |
| | | methylthiazole-2-carboxamide | | | |
| Example 591 Method 106 | ++++ | 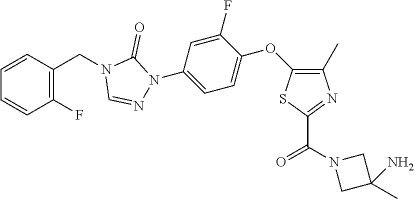 [SMILES: Cc1nc(C(=O)N2CC(C)(N)C2)sc1Oc1ccc(-n2ncn(Cc3ccccc3F)c2=O)cc1F] | white solid, yield: 71.9% | ¹H NMR (400 MHz, Chloroform- d) δ 8.81 (brs, 2H), 7.89-7.81 (m, 1H), 7.69 (d, J = 8.8 Hz, 1H), 7.56 (s, 1H), 7.44-7.30 (m, 2H), 7.19- 7.06 (m, 2H), 7.02 (t, J = 8.8 Hz, 1H), 4.93-4.83 (m, 3H), 4.67- 4.56 (m, 1H), 4.55-4.41 (m, 1H), 4.17-3.93 (m, 1H), 2.25 (s, 3H), 1.68 (s, 3H). | 513.2 |
| | | 2-(4-((2-(3-amino-3-methylazetidine- | | | |
| | | 1-carbonyl)-4-methylthiazol-5- | | | |
| | | yl)oxy)-3-fluorophenyl)-4-(2- | | | |
| | | fluorobenzyl)-2,4-dihydro-3H-1,2,4- | | | |
| | | triazol-3-one | | | |
| Example 592 Method 106 | ++++ | 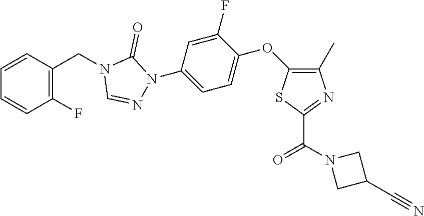 [SMILES: Cc1nc(C(=O)N2CC(C#N)C2)sc1Oc1ccc(-n2ncn(Cc3ccccc3F)c2=O)cc1F] | white solid, yield: 15.7% | ¹H NMR (400 MHz, DMSO-d₆) δ 8.37 (s, 1H), 7.93 (dd, J = 12.4, 2.4 Hz, 1H), 7.74 (dt, J = 9.1, 1.9 Hz, 1H), 7.46-7.34 (m, 3H), 7.29- 7.18 (m, 2H), 4.95 (s, 2H), 4.86 (t, J = 9.6 Hz, 1H), 4.72 (dd, J = 10.0, 6.0 Hz, 1H), 4.35 (t, J = 9.6 Hz, 1H), 4.23 (dd, J = 10.0, 6.0 Hz, 1H), 3.96-3.84 (m, 1H), 2.30 (s, 3H). | 509.2 |
| | | 1-(5-(2-fluoro-4-(4-(2-fluorobenzyl)- | | | |
| | | 5-oxo-4,5-dihydro-1H-1,2,4- | | | |
| | | triazol-1-yl)phenoxy)-4-methylthiazole- | | | |
| | | 2-carbonyl)azetidine-3-carbonitrile | | | |
| Example 593 Method 106 | ++++ | 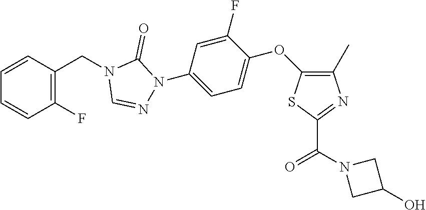 [SMILES: Cc1nc(C(=O)N2CC(O)C2)sc1Oc1ccc(-n2ncn(Cc3ccccc3F)c2=O)cc1F] | white solid, yield: 40.3% | ¹H NMR (400 MHz, DMSO-d₆) δ 8.37 (s, 1H), 7.93 (dd, J = 12.4, 2.4 Hz, 1H), 7.74 (dt, J = 9.2, 2.0 Hz, 1H), 7.46-7.34 (m, 3H), 7.29- 7.19 (m, 2H), 5.78 (d, J = 6.4 Hz, 1H), 4.96 (s, 2H), 4.77 (dd, J = 11.2, 6.4 Hz, 1H), 4.60-4.48 (m, 1H), 4.27 (dd, J = 10.8, 5.6 Hz, 2H), 3.84-3.75 (m, 1H), 2.30 (s, 3H). | 500.2 |
| | | 2-(3-fluoro-4-((2-(3-hydroxyazetidine- | | | |
| | | 1-carbonyl)-4-methylthiazol- | | | |
| | | 5-yl)oxy)phenyl)-4-(2-fluorobenzyl)- | | | |
| | | 2,4-dihydro-3H-1,2,4-triazol-3-one | | | |
| Example 594 Method 106 | +++ | 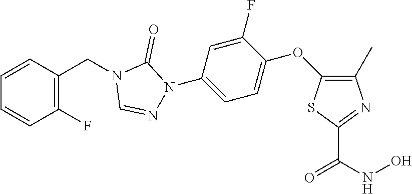 [SMILES: Cc1nc(C(=O)NO)sc1Oc1ccc(-n2ncn(Cc3ccccc3F)c2=O)cc1F] | white solid, yield: 34.7% | ¹H NMR (400 MHz, DMSO-d₆) δ 8.63 (brs, 1H), 8.36 (s, 1H), 7.98- 7.86 (m, 1H), 7.71 (dt, J = 9.2, 2.0 Hz, 1H), 7.45-7.35 (m, 2H), 7.34- 7.19 (m, 3H), 5.32 (brs, 1H), 4.95 (s, 2H), 2.23 (s, 3H). | 460.2 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-N-hydroxy-4- | | | |
| | | methylthiazole-2-carboxamide | | | |
| Example 595 Method 106 | ++++ | 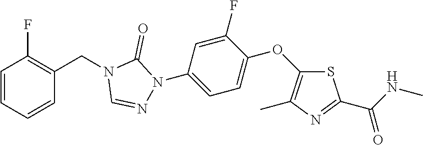 [SMILES: CNC(=O)c1nc(C)c(Oc2ccc(-n3ncn(Cc4ccccc4F)c3=O)cc2F)s1] | white solid, yield: 49.5% | NA | 458.1 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-N,4-dimethylthiazole-2-carboxamide | | | |
| Example 596 Method 106 | ++++ | 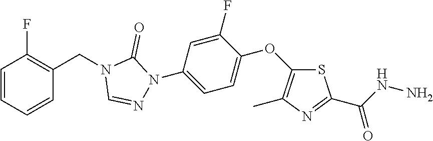 [SMILES: Cc1nc(C(=O)NN)sc1Oc1ccc(-n2ncn(Cc3ccccc3F)c2=O)cc1F] | white solid, yield: 57.2% | ¹H NMR (400 MHz, DMSO-d₆) δ 8.37 (s, 1H), 7.93 (dd, J = 12.8, 2.4 Hz, 1H), 7.77-7.63 (m, 1H), 7.46- 7.29 (m, 3H), 7.30-7.16 (m, 2H), 4.96 (s, 2H), 2.30 (s, 3H). | 459.1 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-4-methylthiazole- | | | |
| | | 2-carbohydrazide | | | |
| Example 597 Method 106 | ++++ | 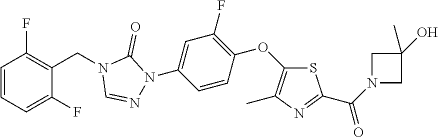 [SMILES: Cc1nc(C(=O)N2CC(C)(O)C2)sc1Oc1ccc(-n2ncn(Cc3c(F)cccc3F)c2=O)cc1F] | Yellow solid, yield: 57.6% | ¹H NMR (400 MHz, DMSO-d₆) δ 8.37 (s, 1H), 7.93 (dd, J = 12.8, 2.4 Hz, 1H), 7.74 (dt, J = 9.2, 2.0 Hz, 1H), 7.46-7.35 (m, 3H), 7.30- 7.18 (m, 2H), 5.72 (s, 1H), 4.96 (s, 2H), 4.45 (d, J = 10.4 Hz, 1H), 4.37 (d, J = 10.4 Hz, 1H), 3.98-3.85 (m, 2H), 2.29 (s, 3H), 1.41 (s, 3H). | 532.2 |
| | | 4-(2,6-difluorobenzyl)-2-(3-fluoro- | | | |
| | | 4-((2-(3-hydroxy-3-methylazetidine-1- | | | |
| | | carbonyl)-4-methylthiazol- | | | |
| | | 5-yl)oxy)phenyl)-2,4-dihydro-3H- | | | |
| | | 1,2,4-triazol-3-one | | | |
| Example 598 Method 106 | ++++ | 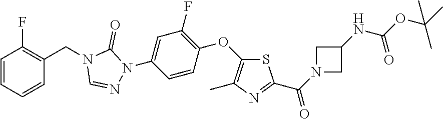 [SMILES: Cc1nc(C(=O)N2CC(NC(=O)OC(C)(C)C)C2)sc1Oc1ccc(-n2ncn(Cc3ccccc3F)c2=O)cc1F] | Yellow solid, yield: 61.2% | NA | 599.2 |
| | | tert-butyl | | | |
| | | (1-(5-(2-fluoro-4-(4-(2-fluorobenzyl)- | | | |
| | | 5-oxo-4,5-dihydro-1H-1,2,4- | | | |
| | | triazol-1-yl)phenoxy)-4-methylthiazole- | | | |
| | | 2-carbonyl)azetidin-3-yl)carbamate | | | |
| Example 599 Method 106 | ++++ | 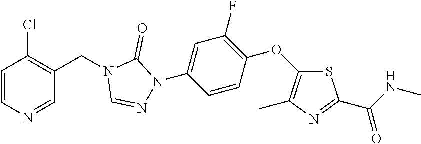 [SMILES: CNC(=O)c1nc(C)c(Oc2ccc(-n3ncn(Cc4cnccc4Cl)c3=O)cc2F)s1] | white solid, yield: 42.9% | ¹H NMR (400 MHz, DMSO-d₆) δ 8.70 (d, J = 5.2 Hz, 1H), 8.63 (s, 1H), 8.54 (d, J = 5.2 Hz, 1H), 8.38 (s, 1H), 7.92 (dd, J = 12.4, 2.4 Hz, 1H), 7.73 (dd, J = 9.2, 2.4 Hz, 1H), 7.62 (d, J = 5.2 Hz, 1H), 5.04 (s, 2H), 3.05 (s, 3H), 2.31 (s, 3H). | 475.8 |
| | | 5-(4-(4-((4-chloropyridin-3-yl) | | | |
| | | methyl)-5-oxo-4,5-dihydro-1H-1,2,4- | | | |
| | | triazol-1-yl)-2-fluorophenoxy)-N,4- | | | |
| | | dimethylthiazole-2-carboxamide | | | |
| Example 600 Method 106 | ++++ | 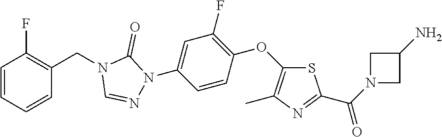 [SMILES: Cc1nc(C(=O)N2CC(N)C2)sc1Oc1ccc(-n2ncn(Cc3ccccc3F)c2=O)cc1F] | white solid, yield: 64.2% | ¹H NMR (400 MHz, DMSO-d₆) δ 8.37 (s, 1H), 8.29 (s, 1H), 7.93 (dd, J = 12.8, 2.4 Hz, 1H), 7.78-7.71 (m, 1H), 7.46-7.34 (m, 3H), 7.30- 7.18 (m, 2H), 4.96 (s, 2H), 4.71 (dd, J = 10.4, 7.6 Hz, 1H), 4.26-4.12 (m, 2H), 3.84-3.73 (m, 1H), 3.68 (dd, J = 10.4, 5.2 Hz, 1H), 2.29 (s, 3H). | 499.1 |
| | | 2-(4-((2-(3-aminoazetidine-1-carbonyl)- | | | |
| | | 4-methylthiazol-5-yl)oxy)-3- | | | |
| | | fluorophenyl)-4-(2-fluorobenzyl)- | | | |
| | | 2,4-dihydro-3H-1,2,4-triazol-3-one | | | |
| Example 601 Method 106 Single unknown stereo- isomer | ++++ | 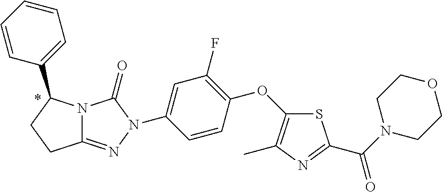 [SMILES: Cc1nc(C(=O)N2CCOCC2)sc1Oc1ccc(-n2nc3n(c2=O)[C@H](c2ccccc2)CC3)cc1F] | white solid, yield: 16.4% | ¹H NMR (400 MHz, DMSO-d₆) δ 7.88 (dd, J = 12.8, 2.4 Hz, 1H), 7.71 (dd, J = 9.2, 2.4 Hz, 1H), 7.40 (dd, J = 11.2, 8.0 Hz, 3H), 7.36-7.30 (m, 3H), 5.32 (dd, J = 7.6, 4.8 Hz, 1H), 4.36 (s, 2H), 3.67 (dd, J = 5.6, 3.6 Hz, 6H), 3.13-2.83 (m, 3H), 2.35 (dd, J = 10.8, 6.4 Hz, 1H), 2.31 (s, 3H). | 522.2 |
| | | (S)-2-(3-fluoro-4-((4-methyl-2- | | | |
| | | (morpholine-4-carbonyl)thiazol-5-yl) | | | |
| | | oxy)phenyl)-5-phenyl-2,5,6,7- | | | |
| | | tetrahydro-3H-pyrrolo[2,1-c][1,2,4] | | | |
| | | triazol-3-one | | | |
| Example 602 Method 106 Single unknown stereo- isomer | ++++ | 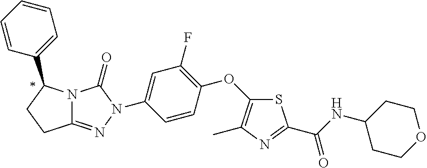 [SMILES: Cc1nc(C(=O)NC2CCOCC2)sc1Oc1ccc(-n2nc3n(c2=O)[C@H](c2ccccc2)CC3)cc1F] | white solid, yield: 21.4% | NA | 536.2 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)-4- | | | |
| | | methyl-N-(tetrahydro-2H-pyran-4- | | | |
| | | yl)thiazole-2-carboxamide | | | |
| Example 603 Method 106 | ++++ | 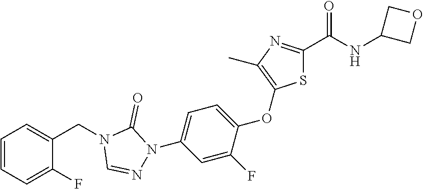 [SMILES: Cc1nc(C(=O)NC2COC2)sc1Oc1ccc(-n2ncn(Cc3ccccc3F)c2=O)cc1F] | Off- white solid, yield: 85.7% | ¹H NMR (400 MHz, DMSO-d₆) δ 9.54 (d, J = 6.8 Hz, 1H), 8.37 (s, 1H), 7.93 (dd, J = 12.6, 2.5 Hz, 1H), 7.74 (dt, J = 9.1, 2.0 Hz, 1H), 7.40 (td, J = 8.2, 7.3, 4.3 Hz, 3H), 7.33-7.15 (m, 2H), 4.96 (s, 2H), 4.68 (dd, J = 7.1, 3.5 Hz, 4H), 2.34 (s, 3H). | 500.1 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-4-methyl-N-(oxetan- | | | |
| | | 3-yl)thiazole-2-carboxamide | | | |
| Example 604 Method 106 | ++++ | 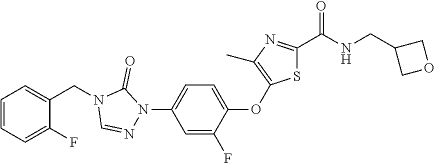 [SMILES: Cc1nc(C(=O)NCC2COC2)sc1Oc1ccc(-n2ncn(Cc3ccccc3F)c2=O)cc1F] | white solid, yield: 68.1% | ¹H NMR (400 MHz, DMSO-d₆) δ 9.01 (t, J = 6.1 Hz, 1H), 8.37 (s, 1H), 7.93 (dd, J = 12.6, 2.6 Hz, 1H), 7.73 (dt, J = 9.1, 1.9 Hz, 1H), 7.50-7.35 (m, 3H), 7.32-7.16 (m, 2H), 4.96 (s, 2H), 4.61 (dd, J = 7.8, 6.1 Hz, 2H), 4.33 (t, J = 6.0 Hz, 2H), 3.52 (t, J = 6.5 Hz, 2H), 3.17 (h, J = 6.9 Hz, 1H), 2.31 (s, 3H). | 514.1 |
| | | 5-(2-fluoro-4-(4-(2-fluorobenzyl)- | | | |
| | | 5-oxo-4,5-dihydro-1H-1,2,4-triazol- | | | |
| | | 1-yl)phenoxy)-4-methyl-N-(oxetan- | | | |
| | | 3-ylmethyl)thiazole-2-carboxamide | | | |
| Example 605 Method 106 | ++++ | 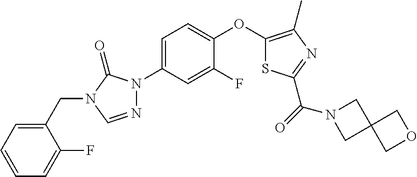 [SMILES: Cc1nc(C(=O)N2CC3(COC3)C2)sc1Oc1ccc(-n2ncn(Cc3ccccc3F)c2=O)cc1F] | white solid, yield: 42.5% | ¹H NMR (400 MHz, DMSO-d₆) δ 8.37 (s, 1H), 7.93 (dd, J = 12.6, 2.5 Hz, 1H), 7.75 (dt, J = 9.1, 1.9 Hz, 1H), 7.40 (qd, J = 8.8, 5.4 Hz, 3H), 7.32-7.12 (m, 2H), 4.96 (s, 2H), 4.81-4.63 (m, 6H), 4.25 (s, 2H), 2.31 (s, 3H). | 526.1 |
| | | 2-(3-fluoro-4-((4-methyl-2-(2-oxa- | | | |
| | | 6-azaspiro[3.3]heptane-6-carbonyl) | | | |
| | | thiazol-5-yl)oxy)phenyl)-4-(2- | | | |
| | | fluorobenzyl)-2,4-dihydro-3H-1,2,4-triazol-3-one | | | |
| Example 606 Method 106 | ++++ | 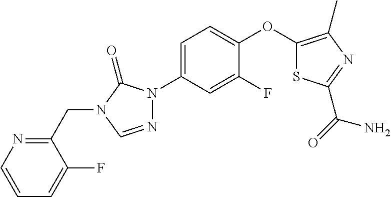 [SMILES: Cc1nc(C(N)=O)sc1Oc1ccc(-n2ncn(Cc3ncccc3F)c2=O)cc1F] | white solid, yield: 23.8% | ¹H NMR (400 MHz, DMSO-d₆) δ 8.40 (s, 1H), 8.10 (s, 1H), 7.94 (dd, J = 12.6, 2.5 Hz, 1H), 7.88-7.71 (m, 3H), 7.56-7.34 (m, 3H), 5.14 (d, J = 1.8 Hz, 2H), 2.31 (s, 3H). | 445.1 |
| | | 5-(2-fluoro-4-(4-((3-fluoropyridin- | | | |
| | | 2-yl)methyl)-5-oxo-4,5-dihydro- | | | |
| | | 1H-1,2,4-triazol-1-yl)phenoxy)-4- | | | |
| | | methylthiazole-2-carboxamide | | | |
| Example 607 Method 106 | ++++ | 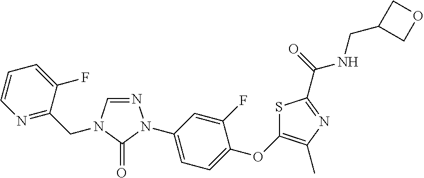 [SMILES: Cc1nc(C(=O)NCC2COC2)sc1Oc1ccc(-n2ncn(Cc3ncccc3F)c2=O)cc1F] | white solid, yield: 31.3% | ¹H NMR (400 MHz, DMSO-d₆) δ 8.84 (t, J = 6.0 Hz, 1H), 8.48-8.34 (m, 2H), 7.94 (dd, J = 12.5, 2.5 Hz, 1H), 7.88-7.68 (m, 2H), 7.60-7.32 (m, 2H), 5.15 (s, 2H), 3.68 (d, J = 5.5 Hz, 2H), 3.48 (ddt, J = 22.1, 10.9, 5.5 Hz, 3H), 3.33 (dp, J = 20.2, 6.8 Hz, 2H), 2.31 (s, 3H). | 515.1 |
| | | 5-(2-fluoro-4-(4-((3-fluoropyridin- | | | |
| | | 2-yl)methyl)-5-oxo-4,5-dihydro- | | | |
| | | 1H-1,2,4-triazol-1-yl)phenoxy)-4- | | | |
| | | methyl-N-(oxetan-3-ylmethyl) | | | |
| | | thiazole-2-carboxamide | | | |
| Example 608 Method 106 | ++++ | 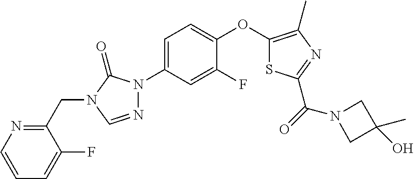 [SMILES: Cc1nc(C(=O)N2CC(C)(O)C2)sc1Oc1ccc(-n2ncn(Cc3ncccc3F)c2=O)cc1F] | Off- white solid, yield: 17.0% | ¹H NMR (400 MHz, DMSO-d₆) δ 8.40-8.36 (m, 2H), 7.94 (dd, J = 12.6, 2.6 Hz, 1H), 7.86-7.71 (m, 2H), 7.55-7.35 (m, 2H), 5.14 (d, J = 1.7 Hz, 2H), 4.51-4.32 (m, 2H), 3.92 (q, J = 10.3 Hz, 2H), 2.30 (s, 3H), 1.41 (s, 3H). | 515.1 |
| | | 2-(3-fluoro-4-((2-(3-hydroxy-3- | | | |
| | | methylazetidine-1-carbonyl)-4- | | | |
| | | methylthiazol-5-yl)oxy)phenyl)-4-((3- | | | |
| | | fluoropyridin-2-yl)methyl)-2,4- | | | |
| | | dihydro-3H-1,2,4-triazol-3-one | | | |
| Example 609 Method 106 | ++++ | 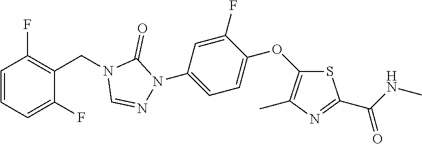 [SMILES: CNC(=O)c1nc(C)c(Oc2ccc(-n3ncn(Cc4c(F)cccc4F)c3=O)cc2F)s1] | white solid, yield: 37.8% | ¹H NMR (400 MHz, DMSO-d₆) δ 8.82-8.70 (m, 1H), 8.41 (s, 1H), 7.95 (dd, J = 12.6, 2.5 Hz, 1H), 7.76 (ddd, J = 9.1, 2.6, 1.4 Hz, 1H), 7.53 (tt, J = 8.5, 6.6 Hz, 1H), 7.42 (t, J = 9.1 Hz, 1H), 7.21 (t, J = 8.1 Hz, 2H), 5.03 (s, 2H), 2.82 (d, J = 4.8 Hz, 3H), 2.35 (s, 3H). | 476.1 |
| | | 5-(4-(4-(2,6-difluorobenzyl)-5- | | | |
| | | oxo-4,5-dihydro-1H-1,2,4-triazol-1- | | | |
| | | yl)-2-fluorophenoxy)-N,4- | | | |
| | | dimethylthiazole-2-carboxamide | | | |
| Example 610 Method 106 | ++++ | 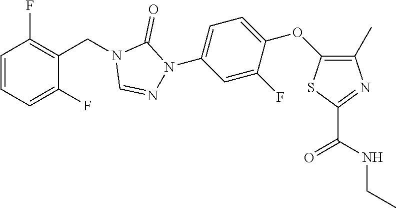 [SMILES: CCNC(=O)c1nc(C)c(Oc2ccc(-n3ncn(Cc4c(F)cccc4F)c3=O)cc2F)s1] | white solid, yield: 53.5% | ¹H NMR (400 MHz, DMSO-d₆) δ 8.77 (t, J = 6.0 Hz, 1H), 8.35 (s, 1H), 7.90 (dd, J = 12.6, 2.6 Hz, 1H), 7.70 (ddd, J = 9.1, 2.6, 1.5 Hz, 1H), 7.48 (tt, J = 8.4, 6.6 Hz, 1H), 7.37 (t, J = 9.1 Hz, 1H), 7.16 (t, J = 8.1 Hz, 2H), 4.98 (s, 2H), 3.30-3.19 (m, 2H), 2.30 (s, 3H), 1.10 (t, J = 7.2 Hz, 3H). | 490.1 |
| | | 5-(4-(4-(2,6-difluorobenzyl)-5- | | | |
| | | oxo-4,5-dihydro-1H-1,2,4-triazol-1- | | | |
| | | yl)-2-fluorophenoxy)-N-ethyl-4- | | | |
| | | methylthiazole-2-carboxamide | | | |
| Example 611 Method 106 | ++++ | 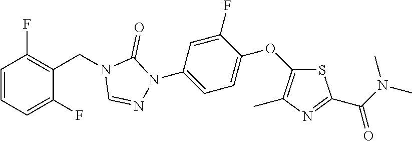 [SMILES: Cc1nc(C(=O)N(C)C)sc1Oc1ccc(-n2ncn(Cc3c(F)cccc3F)c2=O)cc1F] | white solid, yield: 66.2% | ¹H NMR (400 MHz, DMSO-d₆) δ 8.36 (s, 1H), 7.90 (dd, J = 12.6, 2.5 Hz, 1H), 7.72 (ddd, J = 9.1, 2.5, 1.4 Hz, 1H), 7.59-7.45 (m, 1H), 7.40 (t, J = 9.1 Hz, 1H), 7.16 (t, J = 8.1 Hz, 2H), 4.99 (s, 2H), 3.53 (s, 3H), 3.01 (s, 3H), 2.30 (s, 3H). | 490.1 |
| | | 5-(4-(4-(2,6-difluorobenzyl)-5- | | | |
| | | oxo-4,5-dihydro-1H-1,2,4-triazol-1- | | | |
| | | yl)-2-fluorophenoxy)-N,N,4-trimethyl- | | | |
| | | thiazole-2-carboxamide | | | |

### Example 612: Method 107

### tert-butyl (5-((4-(4-benzyl-5-oxo-4,5-dihydro-1H-1,2,4-triazol-1-yl)phenyl)thio)-4-methylthiazol-2-yl)carbamate

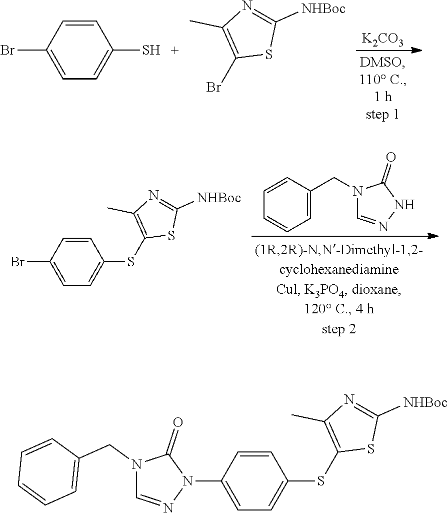

### Step 1: tert-butyl (5-((4-bromophenyl)thio)-4-methylthiazol-2-yl)carbamate

To a solution of 4-bromobenzenethiol (800 mg, 4.23 mmol) in DMSO (15 mL) were added tert-butyl (5-bromo-4-methylthiazol-2-yl) carbamate (1.24 g, 4.23 mmol) and K₂CO₃ (1.75 g, 12.7 mmol). The reaction mixture was stirred at 110° C. for 1 h. The target was detected by LCMS. The reaction was taken up in EtOAc (200 mL) and the organics were washed with 2×100 mL water and then 1×100 mL saturated brine solution. The organics were then separated and dried (MgSO₄) before concentration to dryness. The crude was then purified by flash column chromatography eluted with 10-75% EtOAc in PE. The desired fractions were concentrated to dryness in vacuo. Tert-butyl (5-((4-bromophenyl)thio)-4-methylthiazol-2-yl)carbamate (1.26 g, 74.2% yield) was obtained as a light-yellow solid. MS (m/z): 400.9 [M+H]⁺.

### Step 2: tert-butyl (5-((4-(4-benzyl-5-oxo-4,5-dihydro-1H-1,2,4-triazol-1-yl)phenyl)thio)-4-methylthiazol-2-yl)carbamate

To a solution of tert-butyl (5-((4-bromophenyl)thio)-4-methylthiazol-2-yl)carbamate (163 mg, 496.9 umol) in dioxane (6 mL) were added 4-benzyl-2,4-dihydro-3H-1,2,4-triazol-3-one (100 mg, 496.9 umol), (1R,2R)—N,N′-Dimethyl-1,2-cyclohexanediamine (78 mg, 546.6 umol), CuI (95 mg, 496.9 umol) and K₃PO₄ (317 mg, 1.49 mmol) under Ar. The reaction mixture was stirred at 120° C. for 4 h. The target was detected by LCMS. The reaction was taken up in EtOAc (100 mL) and the organics were washed with 2×50 mL water and then 1×50 mL saturated brine solution. The organics were then separated and dried (MgSO₄) before concentration to dryness. The crude was then purified by flash column chromatography eluted 10-80% EtOAc in PE. The desired fractions were concentrated to dryness in vacuo. Tert-butyl(5-((4-(4-benzyl-5-oxo-4,5-dihydro-1H-1,2,4-triazol-1-yl)phenyl)thio)-4-methylthiazol-2-yl)carbamate (60 mg, 29.8% yield) was obtained as an off-white solid. MS (m/z): 496.2 [M+H]⁺

| | | | | | MS |
|---|---|---|---|---|---|
| Compound | | | Appearance | | (m/z) |
| No. Method | Activity | Structure and Name | and Yield | ¹H NMR Data | [M + H]⁺ |
| Example 612 Method 107 | ++++ | 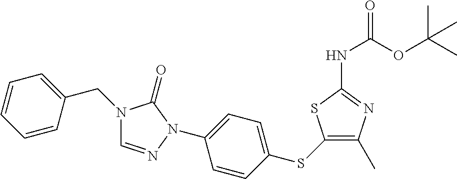 [SMILES: Cc1nc(NC(=O)OC(C)(C)C)sc1Sc1ccc(-n2ncn(Cc3ccccc3)c2=O)cc1] | Off-white solid, yield: 29.8% | NA | 496.2 |
| | | tert-butyl | | | |
| | | (5-((4-(4-benzyl-5-oxo-4,5-dihydro- | | | |
| | | 1H-1,2,4-triazol-1-yl)phenyl) | | | |
| | | thio)-4-methylthiazol-2-yl)carbamate | | | |

### Example 613: Method 108

### 2-(4-((2-(1-aminocyclopropyl)-4-methylthiazol-5-yl)oxy)-3-fluorophenyl)-4-(2,6-difluorobenzyl)-2,4-dihydro-3H-1,2,4-triazol-3-one

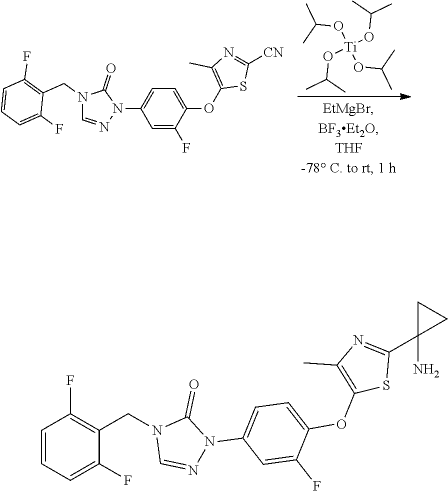

To a solution of 5-[4-[4-[(2,6-difluorophenyl)methyl]-5-oxo-1,2,4-triazol-1-yl]-2-fluoro-phenoxy]-4-methyl-thiazole-2-carbonitrile (300 mg, 0.677 mmol) in THF (30 mL) were added Titanium tetraisopropanolate (212 mg, 0.744 mmol) and ethylmagnesium bromide (180 mg, 1.35 mmol) at −78° C. under Ar. The reaction mixture was stirred at −78° C. for 10 min and then was added boron trifluoride etherate (211 mg, 1.49 mmol). The reaction mixture was allowed to warm to rt and stirred at rt for 1 h. The target was detected by LCMS. The reaction mixture was taken up in EtOAc (200 mL) and the organics were washed with 2×100 mL water and then 1×100 mL saturated brine solution. The organics were then separated and dried (MgSO₄) before concentration to dryness. The crude was then purified by prep-HPLC. 2-[4-[2-(1-aminocyclopropyl)-4-methyl-thiazol-5-yl]oxy-3-fluoro-phenyl]-4-[(2,6-difluorophenyl)methyl]-1,2,4-triazol-3-one (9.0 mg, 2.8% yield) was obtained as a white solid. MS (m/z): 474.1 [M+H]⁺. ¹H NMR (400 MHz, DMSO-d₆) δ 8.47 (s, 1H), 8.33 (s, 1H), 7.85 (dd, J=12.6, 2.6 Hz, 1H), 7.66 (dt, J=9.1, 1.9 Hz, 1H), 7.48 (ddd, J=15.0, 8.5, 6.6 Hz, 1H), 7.16 (td, J=8.6, 8.1, 5.8 Hz, 3H), 4.97 (s, 2H), 2.10 (s, 3H), 1.35-1.03 (m, 4H).

| Compound | | | Appear- | | MS |
|---|---|---|---|---|---|
| No. | | | ance | | (m/z) |
| Method | Activity | Structure and Name | and Yield | ¹H NMR Data | [M + H]⁺ |
| Example 613 Method 108 | ++++ | 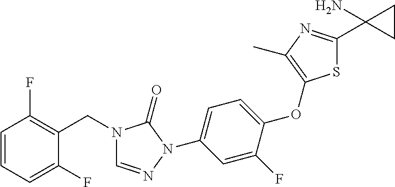 [SMILES: Cc1nc(C2(N)CC2)sc1Oc1ccc(-n2ncn(Cc3c(F)cccc3F)c2=O)cc1F] | white solid, yield: 2.8% | ¹H NMR (400 MHz, DMSO-d₆) δ 8.47 (s, 1H), 8.33 (s, 1H), 7.85 (dd, J = 12.6, 2.6 Hz, 1H), 7.66 (dt, J = 9.1, 1.9 Hz, 1H), 7.48 (ddd, J = 15.0, 8.5, 6.6 Hz, 1H), 7.16 (td, J = 8.6, 8.1, 5.8 Hz, 3H), 4.97 (s, 2H), 2.10 (s, 3H), 1.35-1.03 (m, 4H). | 474.1 |
| | | 2-(4-((2-(1-aminocyclopropyl)- | | | |
| | | 4-methylthiazol-5-yl)oxy)-3- | | | |
| | | fluorophenyl)-4-(2,6-difluorobenzyl)- | | | |
| | | 2,4-dihydro-3H-1,2,4-triazol-3-one | | | |

### Example 614: Method 109

### 2-(4-((2-(3-aminooxetan-3-yl)-4-methylthiazol-5-yl)oxy)-3-fluorophenyl)-4-(2,6-difluorobenzyl)-2,4-dihydro-3H-1,2,4-triazol-3-one

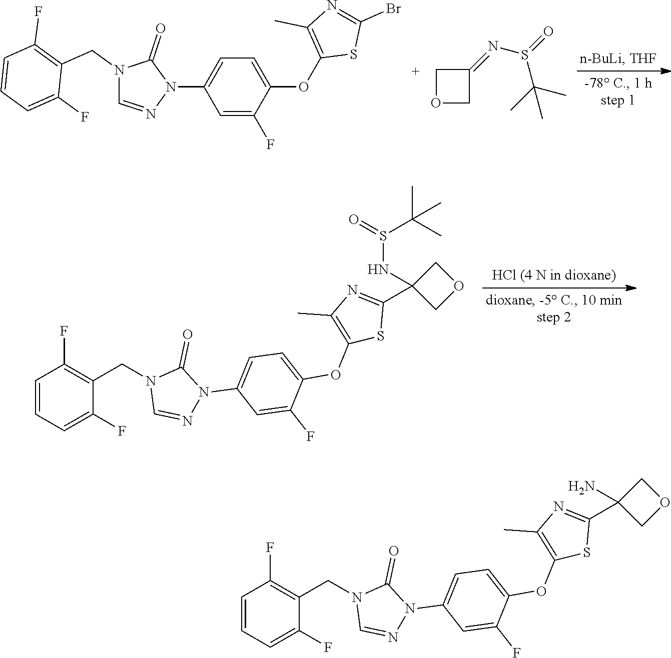 [SMILES: Cc1nc(Br)sc1Oc1ccc(-n2ncn(Cc3c(F)cccc3F)c2=O)cc1F ; CC(C)(C)S(=O)N=C1COC1 ; Cc1nc(C2(NS(=O)C(C)(C)C)COC2)sc1Oc1ccc(-n2ncn(Cc3c(F)cccc3F)c2=O)cc1F ; Cc1nc(C2(N)COC2)sc1Oc1ccc(-n2ncn(Cc3c(F)cccc3F)c2=O)cc1F]

### Step 1: N-(3-(5-(4-(4-(2,6-difluorobenzyl)-5-oxo-4,5-dihydro-1H-1,2,4-triazol-1-yl)-2-fluorophenoxy)-4-methylthiazol-2-yl)oxetan-3-yl)-2-methylpropane-2-sulfinamide

To a solution of 2-[4-(2-bromo-4-methyl-thiazol-5-yl)oxy-3-fluoro-phenyl]-4-[(2,6-difluorophenyl)methyl]-1,2,4-triazol-3-one (200 mg, 0.402 mmol) in THF (20 mL) was added butyllithium (39 mg, 0.603 mmol) at −78° C. under Ar. The reaction mixture was stirred at −78° C. for 5 min and then was added 2-methyl-N-(oxetan-3-ylidene)propane-2-sulfinamide (106 mg, 0.603 mmol). The reaction mixture was allowed to warm to rt and stirred for 1 h. Target was detected by LCMS. The reaction mixture was taken up in EtOAc (200 mL) and the organics were washed with 2×100 mL water and then 1×100 mL saturated brine solution. The organics were then separated and dried (MgSO₄) before concentration to dryness. The crude was then purified by Pre-TLC (PE:EA=2:3). N-[3-[5-[4-[4-[(2,6-difluorophenyl)methyl]-5-oxo-1,2,4-triazol-1-yl]-2-fluoro-phenoxy]-4-methyl-thiazol-2-yl]oxet an-3-yl]-2-methyl-propane-2-sulfinamide (41 mg, 17.2% yield) was obtained as a colorless oil. MS (m/z): 594.1 [M+H]⁺.

### Step 2: 2-(4-((2-(3-aminooxetan-3-yl)-4-methylthiazol-5-yl)oxy)-3-fluorophenyl)-4-(2,6-difluorobenzyl)-2,4-dihydro-3H-1,2,4-triazol-3-one

To a solution of N-[3-[5-[4-[4-[(2,6-difluorophenyl)methyl]-5-oxo-1,2,4-triazol-1-yl]-2-fluoro-phenoxy]-4-methyl-thiazol-2-yl]oxetan-3-yl]-2-methyl-propane-2-sulfinamide (30 mg, 0.0505 mmol) in 1,4-Dioxane (3 mL) was added hydrochloric acid (1 mL, 4 N in dioxane). The reaction mixture was stirred at −5° C. for 10 min. The target was detected by LCMS. 5 mL Et₃N was added slowly at 0° C. and then the reaction mixture was taken up in EtOAc (100 mL) and the organics were washed with 2×50 mL water and then 1×50 mL saturated brine solution. The organics were then separated and dried (MgSO₄) before concentration to dryness. The crude was then purified by reversed-phase column. 2-[4-[2-(3-aminooxetan-3-yl)-4-methyl-thiazol-5-yl]oxy-3-fluoro-phenyl]-4-[(2,6-difluorophenyl)methyl]-1,2,4-triazol-3-one (8.3 mg, 33.6% yield) was obtained as a white solid. MS (m/z): 490.1 [M+H]⁺. ¹H NMR (400 MHz, DMSO) δ 8.34 (s, 1H), 7.86 (dd, J=12.6, 2.6 Hz, 1H), 7.67 (dt, J=9.0, 1.9 Hz, 1H), 7.48 (tt, J=8.5, 6.6 Hz, 1H), 7.27-7.08 (m, 3H), 4.97 (s, 2H), 4.84 (d, J=5.7 Hz, 2H), 4.52 (d, J=5.7 Hz, 2H), 2.25 (s, 3H).

| | | | Appear- | | MS |
|---|---|---|---|---|---|
| | | | ance | | (m/z) |
| Compound | | | and | | [M + |
| No. Method | Activity | Structure and Name | Yield | ¹H NMR Data | H]⁺ |
| Example 614 Method 109 | ++++ | 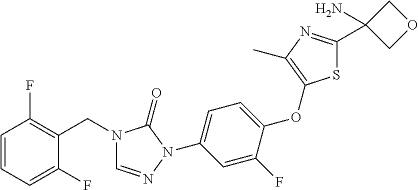 [SMILES: Cc1nc(C2(N)COC2)sc1Oc1ccc(-n2ncn(Cc3c(F)cccc3F)c2=O)cc1F] | white solid, yield 33.6% | ¹H NMR (400 MHz, DMSO-d₆) δ 8.34 (s, 1H), 7.86 (dd, J = 12.6, 2.6 Hz, 1H), 7.67 (dt, J = 9.0, 1.9 Hz, 1H), 7.48 (tt, J = 8.5, 6.6 Hz, 1H), 7.27-7.08 (m, 3H), 4.97 (s, 2H), 4.84 (d, J = 5.7 Hz, 2H), 4.52 (d, J = 5.7 Hz, 2H), 2.25 (s, 3H). | 490.1 |
| | | 2-(4-((2-(3-aminooxetan-3-yl)-4- | | | |
| | | methylthiazol-5-yl)oxy)-3-fluorophenyl)- | | | |
| | | 4-(2,6-difluorobenzyl)- | | | |
| | | 2,4-dihydro-3H-1,2,4-triazol-3-one | | | |

### Example 615: Method 110

### 4-((4-(4-benzyl-5-oxo-4,5-dihydro-1H-1,2,4-triazol-1-yl)phenyl)sulfonyl)picolinonitrile

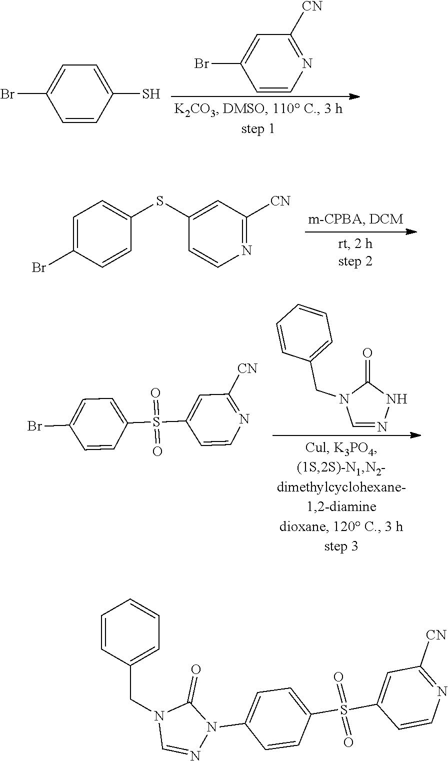

### Step 1: 4-((4-bromophenyl)thio)picolinonitrile

To a solution of 4-bromobenzenethiol (1 g, 5.29 mmol) in DMSO (10 mL) were added 4-bromopicolinonitrile (1.16 g, 6.35 mmol) and K₂CO₃ (2.19 g, 15.9 mmol). The reaction mixture was stirred at 110° C. for 3 h. The target was detected by LCMS. The reaction mixture was taken up in EtOAc (300 mL) and the organics were washed with 2×150 mL water and then 1×150 mL saturated brine solution. The organics were then separated and dried (MgSO₄) before concentration to dryness. The crude was then purified by flash column chromatography eluted with 10-90% EtOAc in PE. The desired fractions were concentrated to dryness in vacuo. 4-((4-bromophenyl)thio)picolinonitrile (1.25 g, 81.2% yield) was obtained as a yellow solid. MS (m/z):291.1 [M+H]⁺.

### Step 2: 4-((4-bromophenyl)sulfonyl)picolinonitrile

To a solution of 4-((4-bromophenyl)thio)picolinonitrile (1.2 g, 4.12 mmol) in DCM (15 mL) was added m-CPBA (2.84 g, 16.5 mmol) slowly. The reaction mixture was stirred at rt for 2 h. The target was detected by LCMS. The reaction mixture was taken up in EtOAc (300 mL) and the organics were washed with 2×150 mL water and then 1×150 mL saturated brine solution. The organics were then separated and dried (MgSO₄) before concentration to dryness. The crude was then purified by flash column chromatography eluted with 10-90% EtOAc in PE. The desired fractions were concentrated to dryness in vacuo. 4-((4-bromophenyl)sulfonyl)picolinonitrile (1.13 g, 84.8% yield) was obtained as a white solid. MS (m/z): 323.1 [M+H]⁺.

### Step 3: 4-((4-(4-benzyl-5-oxo-4,5-dihydro-1H-1,2,4-triazol-1-yl)phenyl)sulfonyl) picolinonitrile

To a solution of 4-benzyl-2,4-dihydro-3H-1,2,4-triazol-3-one (100 mg, 570.8 umol) in dioxane (6 mL) were added 4-((4-bromophenyl)sulfonyl)picolinonitrile (184 mg, 570.8 umol), CuI (109 mg, 570.8 umol), K₃P₄ (364 mg, 1.71 mmol) and (1S,2S)—N¹,N²-dimethylcyclohexane-1,2-diamine (82 mg, 570.8 umol) under Ar. The reaction mixture was stirred at 120° C. for 3 h. The target was detected by LCMS. The reaction mixture was taken up in EtOAc (100 mL) and the organics were washed with 2×50 mL water and then 1×50 mL saturated brine solution. The organics were then separated and dried (MgSO₄) before concentration to dryness. The crude was then purified by Prep-HPLC. 4-((4-(4-benzyl-5-oxo-4,5-dihydro-1H-1,2,4-triazol-1-yl)phenyl)-sulfonyl)picolinonitrile (3 mg, 1.3% yield) was obtained as a white solid. MS (m/z): 418.1 [M+H]⁺. The synthesis procedure of Examples (Compounds) 616-618 were similar to the procedure of Example 615.

| Com- | | | | | |
|---|---|---|---|---|---|
| pound | | | Appearance | | MS |
| No. | Activ- | | and | | (m/z) |
| Method | ity | Structure and Name | Yield | ¹H NMR Data | [M + H]⁺ |
| Example 615 Method 110 | ++++ | 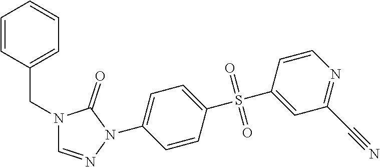 [SMILES: N#Cc1cc(S(=O)(=O)c2ccc(-n3ncn(Cc4ccccc4)c3=O)cc2)ccn1] | White solid, yield: 1.3% | NA | 418.1 |
| | | 4-((4-(4-benzyl-5-oxo-4,5-dihydro-1H- | | | |
| | | 1,2,4-triazol-1-yl)phenyl)sulfonyl)picolinonitrile | | | |
| Example 616 Method 110 | ++++ | 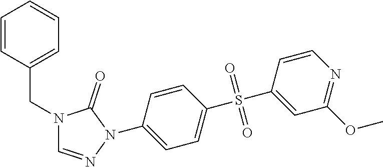 [SMILES: COc1cc(S(=O)(=O)c2ccc(-n3ncn(Cc4ccccc4)c3=O)cc2)ccn1] | White solid, yield: 6.2% | ¹H NMR (400 MHz, CDCl₃) δ 8.30 (d, J = 5.3 Hz, 1H), 8.28- 8.20 (m, 2H), 8.06-7.96 (m, 2H), 7.63 (s, 1H), 7.49 (s, 1H), 7.45-7.19 (m, 6H), 4.86 (s, 2H), 3.96 (s, 3H). | 423.1 |
| | | 4-benzyl-2-(4-((2-methoxypyridin- | | | |
| | | 4-yl)sulfonyl)phenyl)-2,4-dihydro- | | | |
| | | 3H-1,2,4-triazol-3-one | | | |
| Example 617 Method 110 | ++++ | 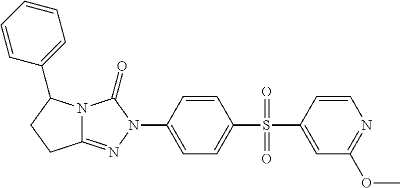 [SMILES: COc1cc(S(=O)(=O)c2ccc(-n3nc4n(c3=O)C(c3ccccc3)CC4)cc2)ccn1] | White solid, yield: 11.2% | ¹H NMR (400 MHz, CDCl₃) δ 8.29 (dd, J = 5.3, 0.7 Hz, 1H), 8.25-8.12 (m, 2H), 8.04-7.90 (m, 2H), 7.50-7.32 (m, 3H), 7.31-7.16 (m, 4H), 5.28 (dd, J = 7.8, 4.3 Hz, 1H), 3.95 (s, 3H), 3.17-2.88 (m, 3H), 2.64-2.43 (m, 1H). | 449.1 |
| | | 2-(4-((2-methoxypyridin-4-yl) | | | |
| | | sulfonyl)phenyl)-5-phenyl-2,5,6,7- | | | |
| | | tetrahydro-3H-pyrrolo[2,1-c][1,2,4] | | | |
| | | triazol-3-one | | | |
| Example 618 Method 110 | ++++ | 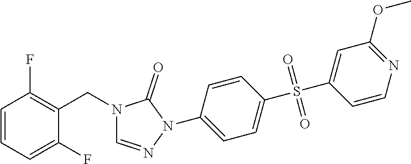 [SMILES: COc1cc(S(=O)(=O)c2ccc(-n3ncn(Cc4c(F)cccc4F)c3=O)cc2)ccn1] | White solid, yield: 8.3% | ¹H NMR (400 MHz, CDCl₃) δ 8.30 (dd, J = 5.3, 0.7 Hz, 1H), 8.27-8.18 (m, 2H), 8.07-7.92 (m, 2H), 7.63 (s, 1H), 7.37 (tt, J = 8.4, 6.5 Hz, 1H), 7.28-7.26 (m, 1H), 7.24-7.18 (m, 1H), 7.05-6.94 (m, 2H), 4.97 (t, J = 1.3 Hz, 2H), 3.95 (s, 3H). | 459.1 |
| | | 4-(2,6-difluorobenzyl)-2-(4-((2- | | | |
| | | methoxypyridin-4-yl)sulfonyl) | | | |
| | | phenyl)-2,4-dihydro-3H-1,2,4-triazol-3-one | | | |

### Example 619: Method 111

### 4-benzyl-2-(4-((4-cyclopentylpiperazin-1-yl)sulfonyl)phenyl)-5-methyl-2,4-dihydro-3H-1,2,4-triazol-3-one

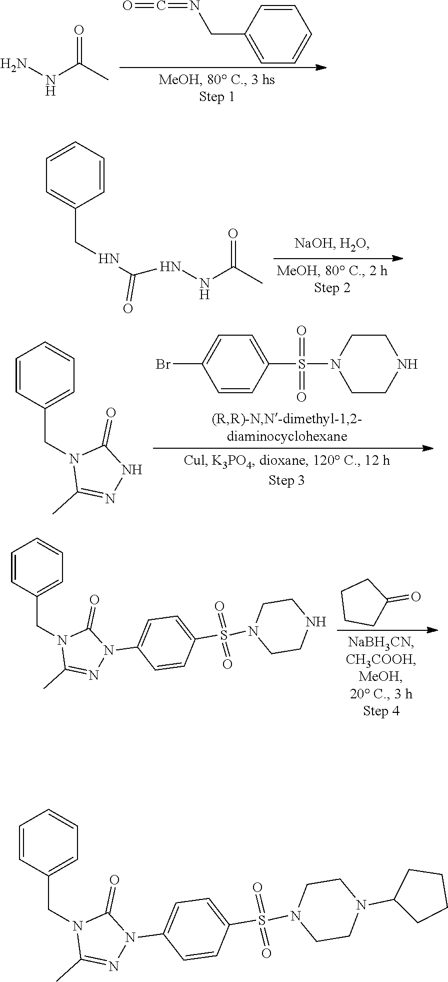

### Step 1 and Step 2: 4-benzyl-5-methyl-2,4-dihydro-3H-1,2,4-triazol-3-one and 4-benzyl-5-methyl-2,4-dihydro-3H-1,2,4-triazol-3-one

The mixture of acetohydrazide (1.8 g, 13.5 mmol) and isocyanatomethyl)benzene (1 g, 13.5 mmol) in ethanol (25 mL) was stirred at 80° C. for 3 hours. After cooling, to the mixture was added 2N NaOH aqueous solution (20 mL). Then the mixture was heated to 80° C., and stirred for 2 hours. After cooling, the mixture was adjusted to pH at 5 with concentrated HCl. The precipitate was collected by filtration, washed with water and dried to give 4-benzyl-5-methyl-2,4-dihydro-3H-1,2,4-triazol-3-one (1.6 g, 2 steps yield: 31%) as a white solid. LC-MS (m/z) 190.1[M+H]⁺. ¹H NMR (400 MHz, CDCl₃) δ ppm 10.5 (br, 1H), 7.30 (m, 5H), 4.83 (s, 2H), 2.11 (s, 3H).

### Step 3: 4-benzyl-5-methyl-2-(4-(piperazin-1-ylsulfonyl)phenyl)-2,4-dihydro-3H-1,2,4-triazol-3-one

To a solution of 4-benzyl-5-methyl-2,4-dihydro-3H-1,2,4-triazol-3-one (1 g, 5.28 mmol) in dioxane (20 mL) was added 1-((4-bromophenyl)sulfonyl)piperazine (1.61 g, 5.28 mmol) and CuI (1 g, 5.28 mmol), (R,R)—N,N′-dimethyl-1,2-diaminocyclohexane (751 mg, 5.28 mmol) and K₃PO₄ (2.24 g, 10.6 mmol) under Ar. The reaction mixture was stirred at 120° C. for 12 h. The target was detected by LCMS. The reaction mixture was taken up in EtOAc (500 mL) and the organics were washed with 2×200 mL water and then 1×200 mL saturated brine solution. The organics were then separated and dried (MgSO₄) before concentration to dryness. The crude was then purified by flash column chromatography eluted with 10-90% EtOAc in PE. The desired fractions were concentrated to dryness in vacuo. 4-benzyl-5-methyl-2-(4-(piperazin-1-ylsulfonyl)phenyl)-2,4-dihydro-3H-1,2,4-triazol-3-one (1.2 g, 54.9% yield) was obtained as a yellow solid. MS (m/z): 414.1 [M+H]⁺.

### Step 4: 4-benzyl-2-(4-((4-cyclopentylpiperazin-1-yl)sulfonyl)phenyl)-5-methyl-2,4-dihydro-3H-1,2,4-triazol-3-one

To a solution of 4-benzyl-5-methyl-2-(4-(piperazin-1-ylsulfonyl)phenyl)-2,4-dihydro-3H-1,2,4-triazol-3-one (100 mg, 241.8 umol) in MeOH (10 mL) were added cyclopentanone (40 mg, 483.7 umol), NaBH₃CN (31 mg, 483.7 umol) and CH₃COOH (44 mg, 725.5 umol) under Ar. The reaction mixture was stirred at 20° C. for 3 h. The target was detected by LCMS. The reaction mixture was taken up in EtOAc (100 mL) and the organics were washed with 2×50 mL water and then 1×50 mL saturated brine solution. The organics were then separated and dried (MgSO₄) before concentration to dryness. The crude was then purified by flash column chromatography eluted with 10-90% EtOAc in PE. The desired fractions were concentrated to dryness in vacuo. 4-benzyl-2-(4-((4-cyclopentylpiperazin-1-yl)sulfonyl)phenyl)-5-methyl-2,4-dihydro-3H-1,2,4-t riazol-3-one (11 mg, 9.4% yield) was obtained as a white solid. MS (m/z): 482.2 [M+H]⁺.

The synthesis procedure of Examples (Compounds) 620-626 was similar to the procedure of Example 619.

| Com- | | | Appear- | | MS |
|---|---|---|---|---|---|
| pound | | | ance | | (m/z) |
| No. | Activ- | | and | | [M + |
| Method | ity | Structure and Name | Yield | ¹H NMR Data | H]⁺ |
| Example 619 Method 111 | + | 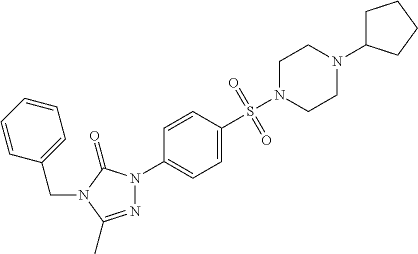 [SMILES: Cc1nn(-c2ccc(S(=O)(=O)N3CCN(C4CCCC4)CC3)cc2)c(=O)n1Cc1ccccc1] | White solid, yield: 9.4% | NA | 482.2 |
| | | 4-benzyl-2-(4-((4-cyclopentylpiperazin-1- | | | |
| | | yl)sulfonyl)phenyl)-5-methyl- | | | |
| | | 2,4-dihydro-3H-1,2,4-triazol-3-one | | | |
| Example 620 Method 111 | ++ | 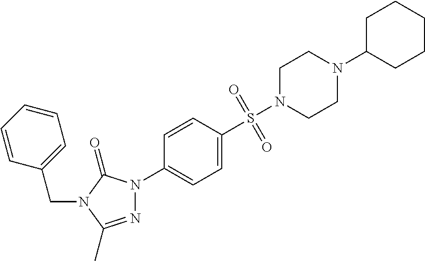 [SMILES: Cc1nn(-c2ccc(S(=O)(=O)N3CCN(C4CCCCC4)CC3)cc2)c(=O)n1Cc1ccccc1] | White solid, yield: 9.4% | NA | 496.2 |
| | | 4-benzyl-2-(4-((4-cyclohexylpiperazin-1- | | | |
| | | yl)sulfonyl)phenyl)-5-methyl- | | | |
| | | 2,4-dihydro-3H-1,2,4-triazol-3-one | | | |
| Example 621 Method 111 | + | 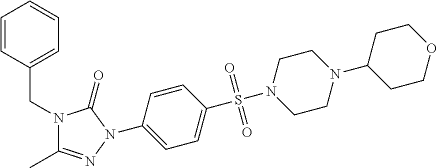 [SMILES: Cc1nn(-c2ccc(S(=O)(=O)N3CCN(C4CCOCC4)CC3)cc2)c(=O)n1Cc1ccccc1] | White solid, yield: 2.5% | NA | 449.8 |
| | | 4-benzyl-5-methyl-2-(4-((4-(tetrahydro- | | | |
| | | 2H-pyran-4-yl)piperazin-1- | | | |
| | | yl)sulfonyl)phenyl)-2,4-dihydro-3H- | | | |
| | | 1,2,4-triazol-3-one | | | |
| Example 622 Method 111 | +++ | 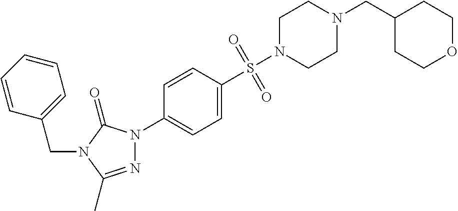 [SMILES: Cc1nn(-c2ccc(S(=O)(=O)N3CCN(CC4CCOCC4)CC3)cc2)c(=O)n1Cc1ccccc1] | White solid, yield: 3.2% | NA | 512.2 |
| | | 4-benzyl-5-methyl-2-(4-((4-((tetrahydro- | | | |
| | | 2H-pyran-4-yl)methyl)piperazin- | | | |
| | | 1-yl)sulfonyl)phenyl)-2,4- | | | |
| | | dihydro-3H-1,2,4-triazol-3-one | | | |
| Example 623 Method 111 | + | 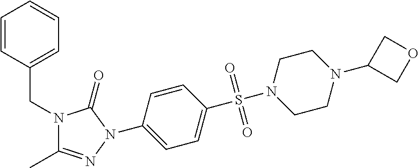 [SMILES: Cc1nn(-c2ccc(S(=O)(=O)N3CCN(C4COC4)CC3)cc2)c(=O)n1Cc1ccccc1] | White solid, yield: 2.6% | NA | 470.2 |
| | | 4-benzyl-5-methyl-2-(4-((4- | | | |
| | | oxetan-3-yl)piperazin-1-yl)sulfonyl) | | | |
| | | phenyl)-2,4-dihydro-3H-1,2,4-triazol-3-one | | | |
| Example 624 Method 111 | ++ | 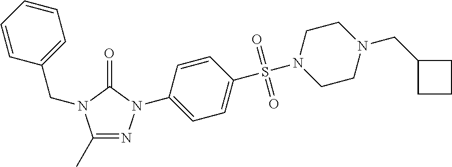 [SMILES: Cc1nn(-c2ccc(S(=O)(=O)N3CCN(CC4CCC4)CC3)cc2)c(=O)n1Cc1ccccc1] | White solid, yield: 1.7% | ¹H NMR (400 MHz, CDCl₃) δ 8.28 (dt, J = 8.8, 3.1 Hz, 2H), 7.82-7.72 (m, 2H), 7.44-7.24 (m, 5H), 4.91 (t, J = 3.1 Hz, 2H), 3.35-2.64 (m, 8H), 2.23 (s, 3H), 2.17-2.09 (m, 2H), 2.02- 1.72 (m, 7H) | 482.2 |
| | | 4-benzyl-2-(4-((4-cyclobutylmethyl) | | | |
| | | piperazin-1-yl)sulfonyl)phenyl)- | | | |
| | | 5-methyl-2,4-dihydro-3H-1,2,4- | | | |
| | | triazol-3-one | | | |
| Example 625 Method 111 | +++ | 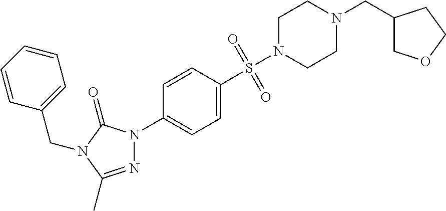 [SMILES: Cc1nn(-c2ccc(S(=O)(=O)N3CCN(CC4CCOC4)CC3)cc2)c(=O)n1Cc1ccccc1] | White solid, yield: 4.2% | ¹H NMR (400 MHz, CDCl₃) δ 8.27 (p, J = 4.9 Hz, 2H), 7.91- 7.72 (m, 2H), 7.44-7.22 (m, 5H), 4.91 (s, 2H), 3.96-3.36 (m, 4H), 3.28-2.46 (m, 10H), 2.24 (s, 3H), 2.10-1.50 (m, 3H). | 498.2 |
| | | 4-benzyl-5-methyl-2-(4-((4- | | | |
| | | ((tetrahydrofuran-3-yl)methyl)piperazin- | | | |
| | | 1-yl)sulfonyl)phenyl)-2,4-dihydro- | | | |
| | | 3H-1,2,4-triazol-3-one | | | |
| Example 626 Method 111 | ++ | 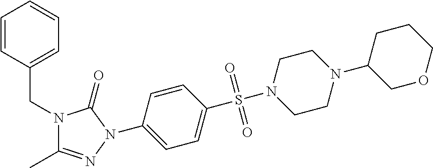 [SMILES: Cc1nn(-c2ccc(S(=O)(=O)N3CCN(C4CCCOC4)CC3)cc2)c(=O)n1Cc1ccccc1] | White solid, yield: 1.7% | NA | 498.2 |
| | | 4-benzyl-5-methyl-2-(4-((4-(tetrahydro- | | | |
| | | 2H-pyran-3-yl)piperazin-1- | | | |
| | | yl)sulfonyl)phenyl)-2,4-dihydro-3H- | | | |
| | | 1,2,4-triazol-3-one | | | |

### Example 627: Method 112

### 2-(4-((2-amino-4-methylthiazol-5-yl)sulfonyl)phenyl)-4-benzyl-2,4-dihydro-3H-1,2,4-triaz ol-3-one

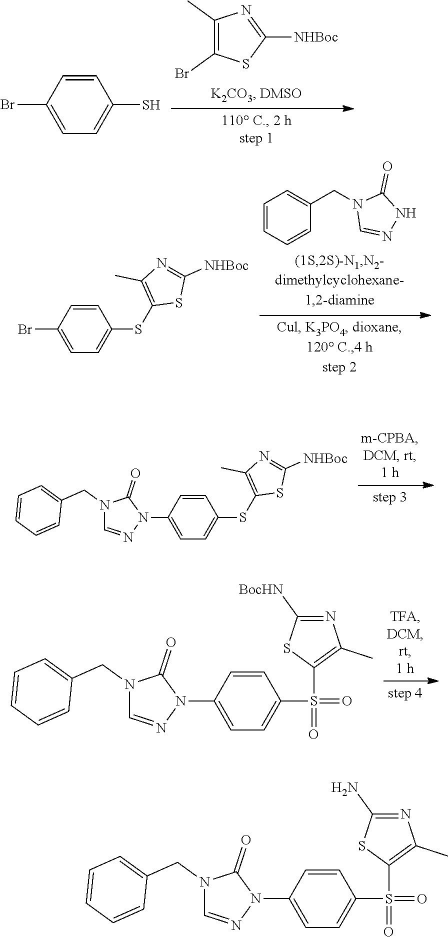

### Step 1: tert-butyl (5-((4-bromophenyl)thio)-4-methylthiazol-2-yl)carbamate

To a solution of 4-bromobenzenethiol (800 mg, 4.23 mmol) in DMSO (15 mL) were added tert-butyl (5-bromo-4-methylthiazol-2-yl)carbamate (1.24 g, 4.23 mmol) and K₂CO₃ (1.75 g, 12.7 mmol). The reaction mixture was stirred at 110° C. for 2 h. The target was detected by LCMS. The reaction mixture was taken up in EtOAc (300 mL) and the organics were washed with 2×150 mL water and then 1×150 mL saturated brine solution. The organics were then separated and dried (MgSO₄) before concentration to dryness. The crude was then purified by flash column chromatography eluted with 10-90% EtOAc in PE. The desired fractions were concentrated to dryness in vacuo. Tert-butyl (5-((4-bromophenyl)thio)-4-methylthiazol-2-yl)carbamate (1.3 g, 76.6% yield) was obtained as a white solid. MS (m/z):401.1 [M+H]⁺.

### Step 2: tert-butyl (5-((4-(4-benzyl-5-oxo-4,5-dihydro-1H-1,2,4-triazol-1-yl)phenyl)thio)-4-methylthiazol-2-yl)carbamate

To a solution of 4-benzyl-2,4-dihydro-3H-1,2,4-triazol-3-one (150 mg, 856.2 umol) in dioxane (10 mL) were added tert-butyl (5-((4-bromophenyl)thio)-4-methylthiazol-2-yl)carbamate (344 mg, 856.2 umol), (1S,2S)—N¹,N²-dimethylcyclohexane-1,2-diamine (134 mg, 941.8 umol) and K₃PO₄ (545 mg, 2.57 mmol) under Ar. The reaction mixture was stirred at 120° C. for 4 h. The target was detected by LCMS. The reaction mixture was taken up in EtOAc (100 mL) and the organics were washed with 2×50 mL water and then 1×50 mL saturated brine solution. The organics were then separated and dried (MgSO₄) before concentration to dryness. The crude was then purified by flash column chromatography eluted with 10-90% EtOAc in PE. The desired fractions were concentrated to dryness in vacuo. Tert-butyl (5-((4-(4-benzyl-5-oxo-4,5-dihydro-1H-1,2,4-triazol-1-yl)phenyl)thio)-4-methylthiazol-2-yl)carbamate (280 mg, 65.9% yield) was obtained as a white solid. MS (m/z):496.1 [M+H]⁺. ¹H NMR (400 MHz, CDCl₃) δ 7.94-7.80 (m, 2H), 7.47-7.30 (m, 6H), 7.25-7.19 (m, 2H), 4.85 (s, 2H), 2.41 (s, 3H), 1.53 (s, 9H).

### Step 3: tert-butyl (5-((4-(4-benzyl-5-oxo-4,5-dihydro-1H-1,2,4-triazol-1-yl)phenyl) sulfonyl)-4-methylthiazol-2-yl)carbamate

To a solution of tert-butyl (5-((4-(4-benzyl-5-oxo-4,5-dihydro-1H-1,2,4-triazol-1-yl)phenyl)thio)-4-methylthiazol-2-yl)carbamate (200 mg, 403.5 umol) in DCM (10 mL) was added m-CPBA (140 mg, 807.1 umol) slowly. The reaction mixture was stirred at r.t. for 1 h. Target was detected by LCMS. The reaction mixture was taken up in EtOAc (100 mL) and the organics were washed with 2×50 mL water and then 1×50 mL saturated brine solution. The organics were then separated and dried (MgSO₄) before concentration to dryness. The crude was then purified by flash column chromatography eluted with 10-90% EtOAc in PE. The desired fractions were concentrated to dryness in vacuo. Tert-butyl (5-((4-(4-benzyl-5-oxo-4,5-dihydro-1H-1,2,4-triazol-1-yl)phenyl)sulfonyl)-4-methylthiazol-2-yl)carbamate (153 mg, 71.9% yield) was obtained as a white solid. MS (m/z): 528.1 [M+H]⁺. ¹H NMR (400 MHz, CDCl₃) δ 8.26-8.13 (m, 2H), 7.97 (td, J=7.0, 2.0 Hz, 2H), 7.82-.66 (m, 1H), 7.49-7.17 (m, 5H), 4.90 (d, J=7.0 Hz, 2H), 2.58-2.36 (m, 3H), 1.54 (d, J=7.0 Hz, 9H).

### Step 4: 2-(4-((2-amino-4-methylthiazol-5-yl)sulfonyl)phenyl)-4-benzyl-2,4-dihydro-3H-1,2,4-triazol-3-one

To a solution of tert-butyl (5-((4-(4-benzyl-5-oxo-4,5-dihydro-1H-1,2,4-triazol-1-yl)phenyl)sulfonyl)-4-methylthiazol-2-yl)carbamate (60 mg, 113.7 umol) in DCM (3 mL) was added TFA (3 mL). The reaction mixture was stirred at r.t. for 1 h. The target was detected by LCMS. The crude was purified by reversed-phase column. 2-(4-((2-amino-4-methylthiazol-5-yl)sulfonyl)phenyl)-4-benzyl-2,4-dihydro-3H-1,2,4-triazol-3-one (27 mg, 55.5% yield) was obtained as a white solid. MS (m/z):428.1 [M+H]⁺. ¹H NMR (400 MHz, CDCl₃) δ 8.19 (dd, J=8.8, 4.8 Hz, 2H), 8.00 (d, J=4.4 Hz, 1H), 7.90 (dd, J 8.7, 4.7 Hz, 2H), 7.75 (d, J=4.4 Hz, 2H), 7.60 (d, J=4.4 Hz, 2H), 7.48-7.16 (i, 1H), 4.90 (s, 2H), 2.35 (d, J=4.5 Hz, 3H).

The synthesis procedure of Examples (Compounds) 628-629 were similar to the procedure of Example 627.

| Com- | | | Appear- | | MS |
|---|---|---|---|---|---|
| pound | | | ance | | (m/z) |
| No. | | | and | | [M + |
| Method | Activity | Structure and Name | Yield | ¹H NMR Data | H]⁺ |
| Example 627 Method 112 | +++ | 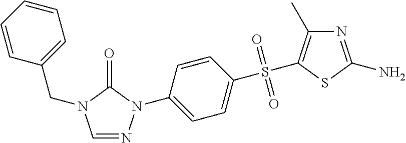 [SMILES: Cc1nc(N)sc1S(=O)(=O)c1ccc(-n2ncn(Cc3ccccc3)c2=O)cc1] | White solid, yield: 55.5% | ¹H NMR (400 MHz, CDCl₃) δ 8.19 (dd, J = 8.8, 4.8 Hz, 2H), 8.00 (d, J = 4.4 Hz, 1H), 7.90 (dd, J = 8.7, 4.7 Hz, 2H), 7.75 (d, J = 4.4 Hz, 2H), 7.60 (d, J = 4.4 Hz, 2H), 7.48- 7.16 (m, 1H), 4.90 (s, 2H), 2.35 (d, J = 4.5 Hz, 3H). | 428.1 |
| | | 2-(4-((2-amino-4-methylthiazol-5- | | | |
| | | yl)sulfonyl)phenyl)-4-benzyl-2,4- | | | |
| | | dihydro-3H-1,2,4-triazol-3-one | | | |
| Example 628 Method 112 | ++++ | 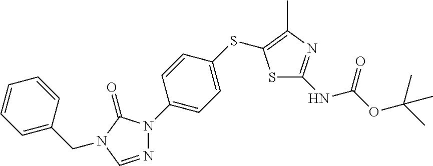 [SMILES: Cc1nc(NC(=O)OC(C)(C)C)sc1Sc1ccc(-n2ncn(Cc3ccccc3)c2=O)cc1] | White solid, yield: 65.9% | ¹H NMR (400 MHz, CDCl₃) δ 7.94-7.80 (m, 2H), 7.47-7.30 (m, 6H), 7.25-7.19 (m, 2H), 4.85 (s, 2H), 2.41 (s, 3H), 1.53 (s, 9H). | 496.1 |
| | | tert-butyl | | | |
| | | (5-((4-(4-benzyl-5-oxo-4,5-dihydro- | | | |
| | | 1H-1,2,4-triazol-1-yl)phenyl)thio)- | | | |
| | | 4-methylthiazol-2-yl)carbamate | | | |
| Example 629 Method 112 | +++ | 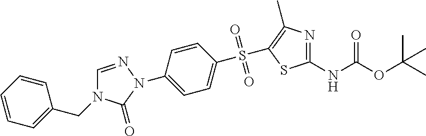 [SMILES: Cc1nc(NC(=O)OC(C)(C)C)sc1S(=O)(=O)c1ccc(-n2ncn(Cc3ccccc3)c2=O)cc1] | White solid, yield: 71.9% | ¹H NMR (400 MHz, CDCl₃) δ 8.26-8.13 (m, 2H), 7.97 (td, J = 7.0, 2.0 Hz, 2H), 7.82-.66 (m, 1H), 7.49-7.17 (m, 5H), 4.90 (d, J = 7.0 Hz, 2H), 2.58-2.36 (m, 3H), 1.54 (d, J = 7.0 Hz, 9H). | 528.1 |
| | | tert-butyl | | | |
| | | (5-((4-(4-benzyl-5-oxo-4,5-dihydro- | | | |
| | | 1H-1,2,4-triazol-1-yl)phenyl) | | | |
| | | sulfonyl)-4-methylthiazol-2-yl)carbamate | | | |

### Example 630: Method 113

### 6-(4-(4-(2,6-difluorobenzyl)-5-oxo-4,5-dihydro-11H-1,2,4-triazol-1-yl)phenoxy)pyrimidine-4-carbonitrile

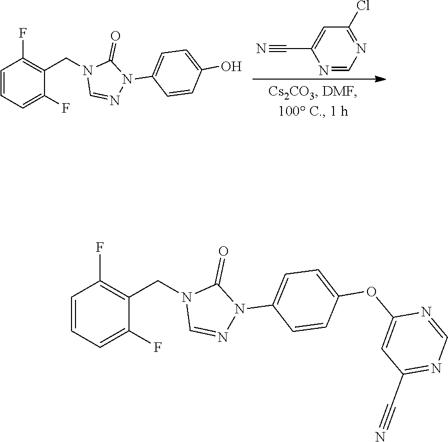

4-(2,6-difluorobenzyl)-2-(4-hydroxyphenyl)-2,4-dihydro-3H-1,2,4-triazol-3-one (303 mg, 1 mmol), 6-chloropyrimidine-4-carbonitrile (153 mg, 1.1 mmol) and Cs₂CO₃ (651.6 mg, 2 mmol) were dissolved in 5 mL DMF. The reaction mixture was taken up in EtOAc (100 mL) and the organic layers were washed with 2×50 mL water and then 1×50 mL saturated brine solution. The organics were then separated and dried (MgSO₄) before concentration to dryness. The crude was then purified by flash column chromatography eluted with 10-90% EtOAc in PE to give the titled compound (39 mg, 9.6% yield) as a white solid. MS (m/z): 407.2 [M+H]⁺. ¹H NMR (400 MHz, DMSO-d₆) δ 8.92 (d, J=1.2 Hz, 1H), 8.33 (s, 1H), 8.00 (d, J=1.1 Hz, 1H), 7.98-7.91 (m, 2H), 7.60-7.43 (m, 1H), 7.42-7.29 (m, 2H), 7.17 (t, J=8.0 Hz, 2H), 4.99 (s, 2H).

| | | | Appear- | | MS |
|---|---|---|---|---|---|
| Compound | | | ance | | (m/z) |
| No. Method | Activity | Structure and Name | and Yield | ¹H NMR Data | [M + H]⁺ |
| Example 630 Method 113 | ++ | 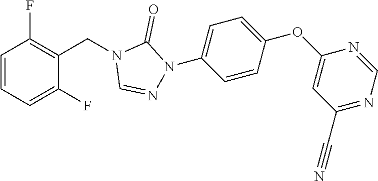 [SMILES: N#Cc1cc(Oc2ccc(-n3ncn(Cc4c(F)cccc4F)c3=O)cc2)ncn1] | white solid, yield: 9.6% | ¹H NMR (400 MHz, DMSO-d₆) δ 8.92 (d, J = 1.2 Hz, 1H), 8.33 (s, 1H), 8.00 (d, J = 1.1 Hz, 1H), 7.98-7.91 (m, 2H), 7.60-7.43 (m, 1H), 7.42-7.29 (m, 2H), 7.17 (t, J = 8.0 Hz, 2H), 4.99 (s, 2H). | 407.2 |
| | | 6-(4-(4-(2,6-difluorobenzyl)-5- | | | |
| | | oxo-4,5-dihydro-1H-1,2,4-triazol- | | | |
| | | 1-yl)phenoxy)pyrimidine-4-carbonitrile | | | |

### Example 631: Method 114

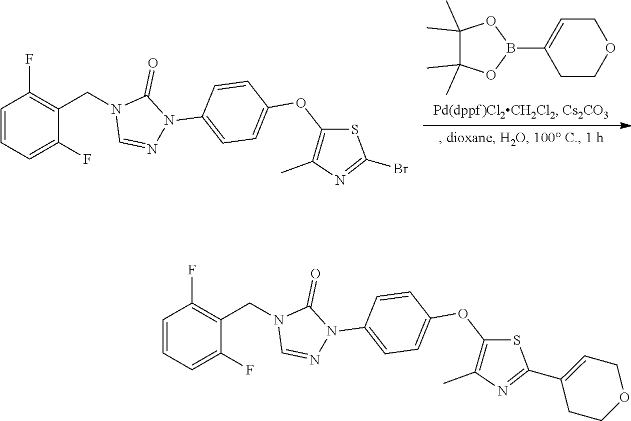 [SMILES: Cc1nc(Br)sc1Oc1ccc(-n2ncn(Cc3c(F)cccc3F)c2=O)cc1 ; CC1(C)OB(C2=CCOCC2)OC1(C)C ; Cc1nc(C2=CCOCC2)sc1Oc1ccc(-n2ncn(Cc3c(F)cccc3F)c2=O)cc1]

To a solution of 2-(Cyclohex-1-en-1-yl)-4,4,5,5-tetramethyl-1,3,2-dioxaborolane (1.50 eq, 65 mg, 0.313 mmol) in DME (10 mL), were added 2-[4-(2-bromo-4-methyl-thiazol-5-yl)oxyphenyl]-4-[(2,6-difluorophenyl)methyl]-1,2,4-t riazol-3-one (1.00 eq, 100 mg, 0.209 mmol), Pd(dppf)Cl₂·CH₂Cl₂ (0.300 eq, 51 mg, 0.0626 mmol), Cs₂CO₃ (2.50 eq, 170 mg, 0.522 mmol), 1,4-Dioxane (10 mL), and Water (1.5 mL). The reaction mixture was stirred for 1.0 h at 100° C. under nitrogen. The reaction was concentrated to dryness and the residue was taken up in EA (50 mL) and the organics were washed with 2×30 mL water and then 1×30 mL saturated brine solution. The organics were then separated and dried (MgSO₄) before concentration to dryness. The crude was then purified by flash column chromatography eluted with 50% EA in PE. The desired fractions were concentrated to dryness in vacuo to afford 4-[(2,6-difluorophenyl)methyl]-2-[4-[2-(3,6-dihydro-2H-pyran-4-yl)-4-methyl-thiazol-5-yl]oxyphenyl]-1,2,4-triazol-3-one (100 mg, 89.40% yield) as a white solid. MS (m/z): 483.2 [M+H]⁺. ¹H NMR (400 MHz, Chloroform-d) δ 7.92 (d, J=7.2 Hz, 2H), 7.57 (s, 1H), 7.36 (tt, J=8.4, 6.4 Hz, 1H), 7.05 (d, J=8.2 Hz, 2H), 7.02-6.94 (m, 2H), 6.65 (s, 1H), 4.97 (d, J=1.2 Hz, 2H), 4.34 (s, 2H), 3.91 (s, 2H), 2.63 (s, 2H), 2.32 (s, 3H).

The synthesis procedure of Examples (Compounds) 632-633 was similar to the procedure of Example 631.

| Com- | | | Appear- | | MS |
|---|---|---|---|---|---|
| pound | Ac- | | ance | | (m/z) |
| No. | tiv- | | and | | [M + |
| Method | ity | Structure and Name | Yield | ¹H NMR Data | H]⁺ |
| Example 631 Method 114 | ++++ | 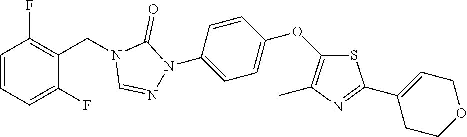 [SMILES: Cc1nc(C2=CCOCC2)sc1Oc1ccc(-n2ncn(Cc3c(F)cccc3F)c2=O)cc1] | White solid, yield: 89.4% | ¹H NMR (400 MHz, Chloroform-d) δ 7.92 (d, J = 7.2 Hz, 2H), 7.57 (s, 1H), 7.36 (tt, J = 8.4, 6.4 Hz, 1H), 7.05 (d, J = 8.2 Hz, 2H), 7.02-6.94 (m, 2H), 6.65 (s, 1H), 4.97 (d, J = 1.2 Hz, 2H), 4.34 (s, 2H), 3.91 (s, 2H), 2.63 (s, 2H), 2.32 (s, 3H). | 483.2 |
| | | 4-(2,6-difluorobenzyl)-2-(4-((2- | | | |
| | | (3,6-dihydro-2H-pyran-4-yl)-4- | | | |
| | | methylthiazol-5-yl)oxy)phenyl)- | | | |
| | | 2,4-dihydro-3H-1,2,4-triazol-3- | | | |
| | | one | | | |
| Example 632 Method 114 | ++++ | 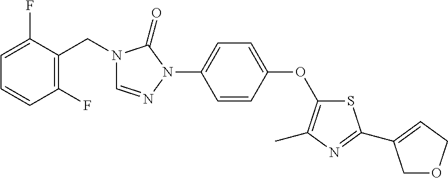 [SMILES: Cc1nc(C2=CCOC2)sc1Oc1ccc(-n2ncn(Cc3c(F)cccc3F)c2=O)cc1] | white solid, yield: 56.5% | NA | 469.2 |
| | | 4-(2,6-difluorobenzyl)-2-(4-((2- | | | |
| | | (2,5-dihydrofuran-3-yl)-4-methylthiazol- | | | |
| | | 5-yl)oxy)phenyl)-2,4-dihydro- | | | |
| | | 3H-1,2,4-triazol-3-one | | | |
| Example 633 Method 114 | ++++ | 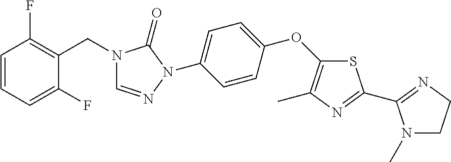 [SMILES: Cc1nc(C2=NCCN2C)sc1Oc1ccc(-n2ncn(Cc3c(F)cccc3F)c2=O)cc1] | white solid, yield: 10.2% | ¹H NMR (400 MHz, DMSO-d₆) δ 8.31 (d, J = 3.6 Hz, 1H), 7.92- 7.83 (m, 2H), 7.48 (tt, J = 8.4, 6.8 Hz, 1H), 7.27-7.11 (m, 4H), 4.98 (s, 2H), 3.92-3.49 (m, 2H), 3.43 (t, J = 10.0 Hz, 2H), 2.51 (p, J = 2.0 Hz, 3H), 2.26 (d, J = 11.2 Hz, 3H). | 483.2 |
| | | 4-(2,6-difluorobenzyl)-2-(4-((4- | | | |
| | | methyl-2-(1-methyl-4,5-dihydro- | | | |
| | | 1H-imidazol-2-yl)thiazol-5-yl) | | | |
| | | oxy)phenyl)-2,4-dihydro-3H-1,2,4- | | | |
| | | triazol-3-one | | | |

### Example 634: Method 115

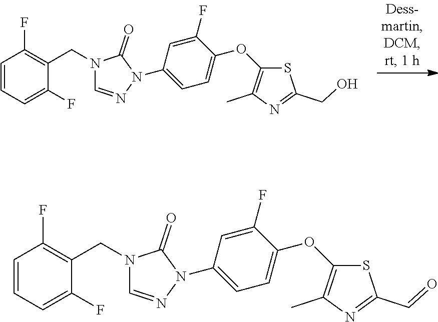 [SMILES: Cc1nc(CO)sc1Oc1ccc(-n2ncn(Cc3c(F)cccc3F)c2=O)cc1F ; Cc1nc(C=O)sc1Oc1ccc(-n2ncn(Cc3c(F)cccc3F)c2=O)cc1F]

To a solution of 4-[(2,6-difluorophenyl)methyl]-2-[3-fluoro-4-[2-(hydroxymethyl)-4-methyl-thiazol-5-yl]oxy-phenyl]-1,2,4-triazol-3-one (1.00 eq, 45 mg, 0.0999 mmol), in DCM (2 mL), Dess-Martin (3.00 eq, 127 mg, 0.300 mmol) was added at room temperature under N₂. After addition, the mixture was stirred at r.t for 3 h. Then the mixture added with water (10 mL) and extracted with EtOAc (10 mL×3). The organics were then combined and dried (Na₂SO₄) before concentration to dryness. The crude was purified via silica chromatography with a gradient of 0-60% EtOAc in PE to give the product 5-[4-[4-[(2,6-difluorophenyl)methyl]-5-oxo-1,2,4-triazol-1-yl]-2-fluoro-phenoxy]-4-m ethyl-thiazole-2-carbaldehyde (18 mg, 21.7% yield) as a white solid. MS (m/z): 447.1[M+H]⁺. ¹H NMR (400 MHz, DMSO-d₆) δ 9.74 (s, 1H), 7.91-7.80 (m, 2H), 7.48 (tt, J=8.4, 6.8 Hz, 1H), 7.19-7.09 (m, 4H), 4.98 (s, 2H), 2.22 (s, 3H).

| Compound | | | Appearance | | MS(m/z) |
|---|---|---|---|---|---|
| No. Method | Activity | Structure and Name | and Yield | ¹H NMR Data | [M + H]⁺ |
| Example 634 Method 115 | ++++ | 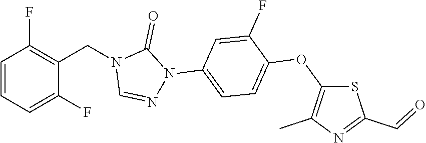 [SMILES: Cc1nc(C=O)sc1Oc1ccc(-n2ncn(Cc3c(F)cccc3F)c2=O)cc1F] 5-(4-(4-(2,6-difluorobenzyl)-5-oxo-4,5-dihydro-1H-1,2,4-triazol-1-yl)- 2-fluorophenoxy)-4-methylthiazole-2-carbaldehyde | white solid, yield: 21.7% | ¹H NMR (400 MHz, DMSO-d₆) δ 9.74 (s, 1H), 7.91-7.80 (m, 2H), 7.48 (tt, J = 8.4, 6.8 Hz, 1H), 7.19-7.09 (m, 4H), 4.98 (s, 2H), 2.22 (s, 3H). | 447.1 |

### Example 635: Method 116

### (S)-4-methyl-5-((4-(3-oxo-5-phenyl-6,7-dihydro-3H-pyrrolo[2,1-c][1,2,4]triazol-2(5H)-yl)cyclohexyl)oxy)thiazole-2-carboxamide

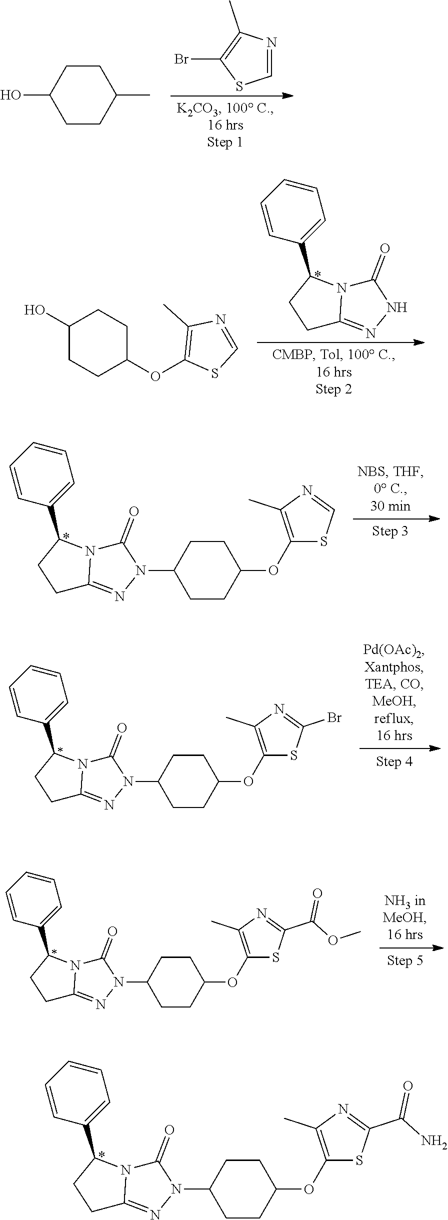

### Step 1: 4-((4-methylthiazol-5-yl)oxy)cyclohexan-1-ol

The mixture of 4-methylcyclohexan-1-ol (4 g, 35 mmol), 5-bromo-4-methylthiazole (6.23 g, 35 mmol) and Potassium carbonate (6.9 g, 50 mmol) was stirred at 100° C. for 16 hrs. After cooling, the residue was purified by column chromatography on silica gel eluted with (Petroleum ether: Ethyl acetate=10:1 to 3:1) to give 4-((4-methylthiazol-5-yl)oxy)cyclohexan-1-ol (300 mg, yield: 4%) as white solid. LC-MS (m/z): 214.1 [M+H]⁺.

### Step 2: (S)-2-(4-((4-methylthiazol-5-yl)oxy)cyclohexyl)-5-phenyl-2,5,6,7-tetrahydro-3H-pyrrolo[2,1-c][1,2,4]triazol-3-one

The mixture of 4-((4-methylthiazol-5-yl)oxy)cyclohexan-1-ol (300 mg, 1.4 mmol), (S)-5-phenyl-2,5,6,7-tetrahydro-3H-pyrrolo[2,1-c][1,2,4]triazol-3-one (300 mg, 1.4 mmol) and CMBP (390 mg, 1.6 mmol) in dried toluene (10 mL) was stirred at 100° C. for 16 hours under nitrogen atmosphere. After cooling, the solvent was removed in vacuo. The residue was purified by column chromatography on silica gel (eluting methanol/dichloromethane 1:100˜3:100) to give (S)-2-(4-((4-methylthiazol-5-yl)oxy)cyclohexyl)-5-phenyl-2,5,6,7-tetrahydro-3H-pyrrolo[2,1-c][1,2,4]triazol-3-one (400 mg contained 50% of tributylphosphine) as a brown oil.

### Step 3: (S)-2-(4-((2-bromo-4-methylthiazol-5-yl)oxy)cyclohexyl)-5-phenyl-2,5,6,7-tetrahydro-3H-pyrrolo[2,1-c][1,2,4]triazol-3-one

At 0° C., to the solution of (S)-2-(4-((4-methylthiazol-5-yl)oxy)cyclohexyl)-5-phenyl-2,5,6,7-tetrahydro-3H-pyrrolo[2,1-c][1,2,4]triazol-3-one (400 mg containing 50% of tributylphosphine) in THF (5 mL) was added dropwise NBS (112 mg, 0.63 mmol) in THF (3 mL). After addition, the mixture was stirred at 0° C. for 30 min. The solvent was removed in vacuo. The residue was purified by column chromatography on silica gel (eluting methanol/dichloromethane 1:100˜2:100) to give (S)-2-(4-((2-bromo-4-methylthiazol-5-yl)oxy)cyclohexyl)-5-phenyl-2,5,6,7-tetrahydro-3H-pyrrolo[2,1-c][1,2,4]triazol-3-one (90 mg, yield: 38%) as a yellow oil.

### Step 4: methyl (S)-4-methyl-5-((4-(3-oxo-5-phenyl-6,7-dihydro-3H-pyrrolo[2,1-c][1,2,4]triazol-2(5H)-yl)cyclohexyl)oxy)thiazole-2-carboxylate

The mixture of (S)-2-(4-((2-bromo-4-methylthiazol-5-yl)oxy)cyclohexyl)-5-phenyl-2,5,6,7-tetrahydro-3H-pyrrolo[2,1-c][1,2,4]triazol-3-one (90 mg, 0.2 mmol), palladium (II) acetate (11 mg, 0.01 mmol) and xantphos (12 mg, 0.02 mmol) in MeOH (5 mL) was stirred at reflux for 16 hours under CO balloon. The solvent was removed to give a crude product without further purification.

### Step 5: (S)-4-methyl-5-((4-(3-oxo-5-phenyl-6,7-dihydro-3H-pyrrolo[2,1-c][1,2,4]triazol-2(5H)-yl)cyclohexyl)oxy)thiazole-2-carboxamide

To the crude (S)-4-methyl-5-((4-(3-oxo-5-phenyl-6,7-dihydro-3H-pyrrolo[2,1-c][1,2,4]triazol-2(5H)-yl)cyclohexyl)oxy)thiazole-2-carboxylate was added amine/methanol solution (3 mL), and then was sealed. The mixture was stirred at 70° C. for 16 hrs. The solvent was removed in vacuo. The residue was purified by Prep-HPLC (Gemini-C₁₈ 150×21.2 mm, 5 um, eluting: ACN-H₂O (0.10% FA), gradient: 30-70). The product was frozen with liquid nitrogen, and lyophilized overnight to give (S)-4-methyl-5-((4-(3-oxo-5-phenyl-6,7-dihydro-3H-pyrrolo[2,1-c][1,2,4]triazol-2(5H)-yl)cyclohexyl)oxy)thiazole-2-carboxamide (1.4 mg, 2 steps yield: 2%). LC-MS (m/z): 440.1 [M+H]⁺. ¹H NMR (400 MHz, CDCl₃) δ ppm 7.35 (m, 3H), 7.17 (m, 2H), 6.02 (s, 1H), 5.27 (s, 1H), 4.30 (m, 1H), 4.16 (m, 1H), 3.10-2.78 (m, 3H), 2.55-2.44 (m, 1H), 2.36 (s, 3H), 2.31-2.14 (m, 4H), 1.80 (m, 4H).

The synthesis procedure of Example (Compound) 636 was similar to the procedure of Example 635.

| Compound | | | Appearance | | MS(m/z) |
|---|---|---|---|---|---|
| No. Method | Activity | Structure and Name | and Yield | ¹H NMR Data | [M + H]⁺ |
| Example 635 Method 116 single unknown stereoisomer | ++ | 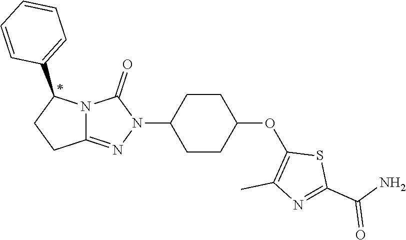 [SMILES: Cc1nc(C(N)=O)sc1OC1CCC(n2nc3n(c2=O)[C@H](c2ccccc2)CC3)CC1] (S)-4-methyl-5-((4-(3-oxo-5-phenyl-6,7-dihydro-3H-pyrrolo[2,1-c] [1,2,4]triazol-2(5H)-yl)cyclohexyl)oxy)thiazole-2-carboxamide | white solid, yield: 2% | ¹H NMR (400 MHZ, CDCl₃) δ ppm 7.35 (m, 3H), 7.17 (m, 2H), 6.02 (s, 1H), 5.27 (s, 1H), 4.30 (m, 1H), 4.16 (m, 1H), 3.10-2.78 (m, 3H), 2.55-2.44 (m, 1H), 2.36 (s, 3H), 2.31-2.14 (m, 4H), 1.80 (m, 4H). | 440.1 |
| Example 636 Method 116 single unknown stereoisomer | +++ | 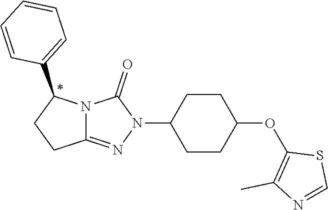 [SMILES: Cc1ncsc1OC1CCC(n2nc3n(c2=O)[C@H](c2ccccc2)CC3)CC1] (S)-2-(4-((4-methylthiazol-5-yl)oxy)cyclohexyl)-5-phenyl- 2,5,6,7-tetrahydro-3H-pyrrolo[2,1-c][1,2,4]triazol-3-one | white solid, yield: 29% | ¹H NMR (400 MHZ, CDCl₃) δ 8.72 (s, 1H), 7.32-7.39 (m, 3H), 7.21-7.09 (m, 2H), 5.26 (dd, J = 7.9, 4.0 Hz, 1H), 4.12-4.16 (m, 1H), 3.96-3.98 (m, 1H), 2.81-3.03 (m, 3H), 2.55- 2.43 (m, 1H), 2.36 (s, 3H), 2.29-2.34 (m, 2H), 2.14- 2.01 (m, 2H), 1.84-19.4 (m, 2H), 1.67-1.73 (m, 2H) | 397.1 |

### Example 637: Method 117

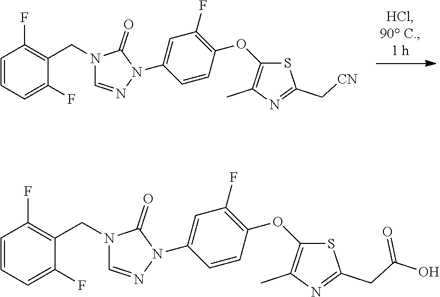 [SMILES: Cc1nc(CC#N)sc1Oc1ccc(-n2ncn(Cc3c(F)cccc3F)c2=O)cc1F ; Cc1nc(CC(=O)O)sc1Oc1ccc(-n2ncn(Cc3c(F)cccc3F)c2=O)cc1F]

A solution of 2-[5-[4-[4-[(2,6-difluorophenyl)methyl]-5-oxo-1,2,4-triazol-1-yl]-2-fluoro-phenoxy]-4-methyl-thiazol-2-yl]acetonitrile (1.00 eq, 100 mg, 0.219 mmol) in HCl (6 M/L, 3.0 mL) was stirred at 90° C. for 3 h. Then the mixture was added with water (10 mL) and extracted with EtOAc (5 mL×3), washed with brine (10 mL). The organics were then combined and dried (Na₂SO₄) before concentration to dryness. The crude was purified with Flash (PE/EtOAc=1:1) to give the product 2-[5-[4-[4-[(2,6-difluorophenyl)methyl]-5-oxo-1,2,4-triazol-1-yl]-2-fluoro-phenoxy]-4-methyl-thiazol-2-yl]acetic acid (20 mg, 18.2500 yield) as a white solid. MS (m/z): 477.2 [M+H]⁺. ¹H NMR (400 MHz, DMSO-d₆) δ 8.34 (s, 1H), 7.87 (dd, J=12.8, 2.8 Hz, 1H), 7.67 (d, J=2.0 Hz, 1H), 7.54-7.42 (m, 1H), 7.19 (dt, J=26.0, 8.8 Hz, 3H), 4.97 (s, 2H), 3.94 (s, 2H), 2.19 (s, 3H).

| Compound | | | Appear- | | |
|---|---|---|---|---|---|
| No. | | | ance | | MS(m/z) |
| Method | Activity | Structure and Name | and Yield | ¹H NMR Data | [M + H]⁺ |
| Example 637 Method 117 | ++++ | 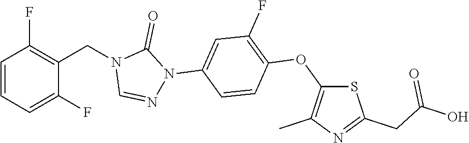 [SMILES: Cc1nc(CC(=O)O)sc1Oc1ccc(-n2ncn(Cc3c(F)cccc3F)c2=O)cc1F] 2-(5-(4-(4-(2,6-difluorobenzyl)-5-oxo-4,5-dihydro-1H-1,2,4-triazol-1-yl)- 2-fluorophenoxy)-4-methylthiazol-2-yl)acetic acid | white solid, yield: 10.8% | ¹H NMR (400 MHZ, DMSO-d6) δ 8.34 (s, 1H), 7.87 (dd, J = 12.8, 2.8 Hz, 1H), 7.67 (d, J = 2.0 Hz, 1H), 7.54-7.42 (m, 1H), 7.22 (t, J = 9.2 Hz, 1H), 7.16 (t, J = 8.0 Hz, 2H), 4.97 (s, 2H), 3.94 (s, 2H), 2.19 (s, 3H). | 477.0 |

### Example 638: Method 118

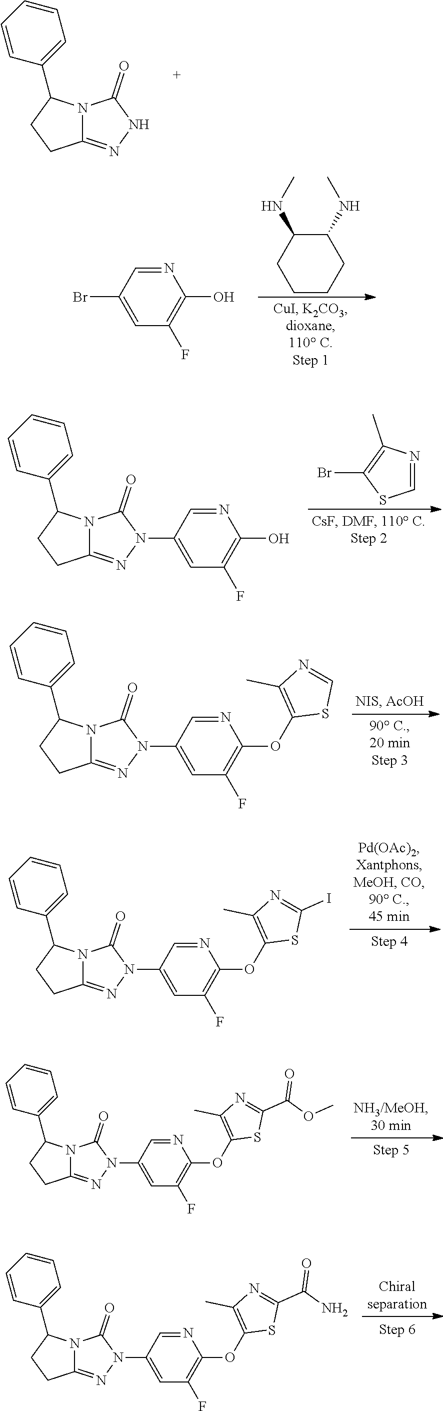

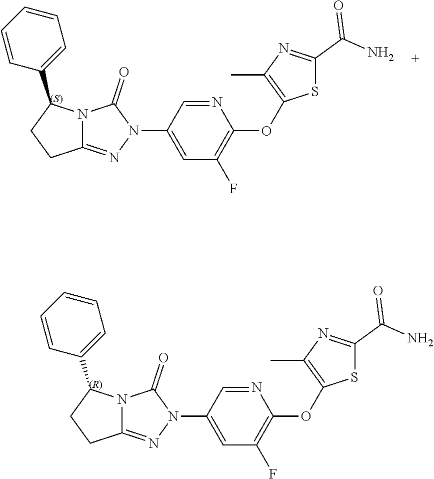

### Step 1: 2-(5-fluoro-6-hydroxypyridin-3-yl)-5-phenyl-2,5,6,7-tetrahydro-3H-pyrrolo [2,1-c][1,2,4]triazol-3-one

To a stirred solution of 5-phenyl-2,5,6,7-tetrahydropyrrolo[2,1-c][1,2,4]triazol-3-one (1.00 eq, 1500 mg, 7.45 mmol), 5-bromo-3-fluoro-pyridin-2-ol (1.00 eq, 1431 mg, 7.45 mmol), (1R,2R)—N,N′-Dimethyl-1,2-cyclohexanediamine (1.00 eq, 1060 mg, 7.45 mmol), K₂CO₃ (3.00 eq, 3086 mg, 22.40 mmol) in DMF (30 mL) was added CuI (1.00 eq, 1420 mg, 7.45 mmol) at room temperature under N₂. After addition, the mixture was stirred at 110° C. for 1 h. Then the mixture was added with water (20 mL) and extracted with EtOAc (10 mL×3), washed with brine (10 mL×5). The organics were then combined and dried (Na₂SO₄) before concentration to dryness. The crude was purified with Flash (PE/EtOAc=1:1) to give the product 2-(5-fluoro-6-hydroxy-3-pyridyl)-5-phenyl-6,7-dihydro-5H-pyrrolo[2,1-c][1,2,4]triazol-3-one (1002 mg, 38.66% yield) as a yellow solid. MS (m/z): 313.3 [M+H]⁺.

### Step 2: 2-(5-fluoro-6-((4-methylthiazol-5-yl)oxy)pyridin-3-yl)-5-phenyl-2,5,6,7-tetrahydro-3H-pyrrolo[2,1-c][1,2,4]triazol-3-one

To a solution of 2-(5-fluoro-6-hydroxy-3-pyridyl)-5-phenyl-6,7-dihydro-5H-pyrrolo 20 [2,1-c][1,2,4]triazol-3-one (1.00 eq, 1000 mg, 3.20 mmol), and 5-Bromo-4-methylthiazole (1.00 eq, 570 mg, 3.20 mmol) in NMP (15 mL), CsF (2.00 eq, 973 mg, 6.40 mmol) was added at room temperature under N₂. After addition, the mixture was stirred at 110° C. for 1 h. Then the mixture was added with water (20 mL) and extracted with EtOAc (10 mL×3) washed with brine (10 mL×5). The organics were then combined and dried (Na₂SO₄) before concentration to dryness. The crude was purified with flash (PE/EtOAc=1:1) to give the product 2-(5-fluoro-6-((4-methylthiazol-5-yl)oxy)pyridin-3-yl)-5-phenyl-2,5,6,7-tetrahydro-3H-pyrrolo[2,1-c][1,2,4]triazol-3-one (800 mg, 54.31% yield) as a white solid. MS (m/z): 409.4 [M+H]⁺.

### Step 3: 2-(5-fluoro-6-((2-iodo-4-methylthiazol-5-yl)oxy)pyridin-3-yl)-5-phenyl-2,5,6,7-tetrahydro-3H-pyrrolo[2,1-c][1,2,4]triazol-3-one

To a solution of 2-(5-fluoro-6-((4-methylthiazol-5-yl)oxy)pyridin-3-yl)-5-phenyl-2,5,6,7-tetrahydro-3H-pyrrolo[2,1-c][1,2,4]triazol-3-one (1.00 eq, 400 mg, 0.979 mmol) in Acetic acid (10 mL), NIS (2.00 eq, 1441 mg, 6.40 mmol) was added at room temperature under N₂. After addition, the mixture was stirred at 90° C. for 1 h. Then the mixture was added with water (10 mL) and extracted with EtOAc (10 mL×3). The organics were then combined and dried (Na₂SO₄) before concentration to dryness. The crude was purified via silica chromatography with a gradient of 0-60% EtOAc in PE to give the product 2-[5-fluoro-6-(2-iodo-4-methyl-thiazol-5-yl)oxy-3-pyridyl]-5-phenyl-6,7-dihydro-5H-pyrrolo[2,1-c][1,2,4]triazol-3-one (350 mg, 63.79% yield) as a yellow solid. LC-MS (m/z) 536.3[M+H]⁺.

### Step 4: Methyl 5-((3-fluoro-5-(3-oxo-5-phenyl-6,7-dihydro-3H-pyrrolo[2,1-c][1,2,4]triazol-2(5H)-yl)pyridin-2-yl)oxy)-4-methylthiazole-2-carboxylate

To a solution of 2-[5-fluoro-6-(2-iodo-4-methyl-thiazol-5-yl)oxy-3-pyridyl]-5-phenyl-6,7-dihydro-5H-pyrrolo[2,1-c][1,2,4]triazol-3-one (1.00 eq, 350 mg, 0.654 mmol), Xantphos (0.400 eq, 151 mg, 0.262 mmol), and Et₃N (3.00 eq, 198 mg, 1.96 mmol) in methanol (5 mL), Pd(OAc)₂ (0.200 eq, 29 mg, 0.131 mmol) was added at room temperature under CO. After addition, the mixture was stirred at 70° C. for 2 h. Then the mixture was added with water (5 mL) and extracted with EtOAc (4 mL×3). The organics were then combined and dried (Na₂SO₄) before concentration to dryness. The crude was purified with prep-TLC (PE/EtOAc=1:1) to give the product methyl 5-((3-fluoro-5-(3-oxo-5-phenyl-6,7-dihydro-3H-pyrrolo [2,1-c][1,2,4]triazol-2(5H)-yl)pyridin-2-yl)oxy)-4-methylthiazole-2-carboxylate (200 mg, 62.17% yield) as a black solid. MS (m/z): 468.5 [M+H]⁺.

### Step 5: 5-((3-fluoro-5-(3-oxo-5-phenyl-6,7-dihydro-3H-pyrrolo[2,1-c][1,2,4]triazol-2(5H)-yl)pyridin-2-yl)oxy)-4-methylthiazole-2-carboxamide

A solution of 5-((3-fluoro-5-(3-oxo-5-phenyl-6,7-dihydro-3H-pyrrolo[2,1-c][1,2,4]triazol-2(5H)-yl)pyridin-2-yl)oxy)-4-methylthiazole-2-carboxylate (1.00 eq, 200 mg, 0.428 mmol) in NH₃ (7M in MeOH) (5 mL) was stirred at 70° C. for 1 h. The reaction was concentrated to dryness then triturated with MeOH to give 5-((3-fluoro-5-(3-oxo-5-phenyl-6,7-dihydro-3H-pyrrolo[2,1-c][1,2,4]triazol-2(5H)-yl)pyridin-2-yl)oxy)-4-methylthiazole-2-carboxamide (120 mg, 59.51% yield). MS (m/z): 453.5 [M+H]⁺.

Step 6: the product from step 5 (120 mg) was purified by chiral separation by SFC under basic condition to give two products.

### (S)-5-((3-fluoro-5-(3-oxo-5-phenyl-6,7-dihydro-3H-pyrrolo[2,1-c][1,2,4]triazol-2(5H)-yl)pyridin-2-yl)oxy)-4-methylthiazole-2- carboxamide

40 mg white solid, yield: 33.3%. MS (m/z): 453.5 [M+H]⁺. ¹H NMR (400 MHz, Chloroform-d) δ 8.72 (d, J=2.4 Hz, 1H), 8.60 (d, J=2.4 Hz, 1H), 7.58 (brs, 1H), 7.46-7.32 (m, 3H), 7.28-7.26 (m, 2H), 5.29 (dd, J=8.0, 4.4 Hz, 1H), 3.15-3.01 (m, 2H), 3.01-2.89 (m, 1H), 2.62-2.52 (m, 4H).

### (R)-5-((3-fluoro-5-(3-oxo-5-phenyl-6,7-dihydro-3H-pyrrolo[2,1-c][1,2,4]triazol-2(5H)-yl)pyridin-2-yl)oxy)-4-methylthiazole-2- carboxamide

42 mg white solid, yield: 35%. MS (m/z): 453.5 [M+H]⁺. ¹H NMR (400 MHz, Chloroform-d) δ 8.74 (d, J=2.4 Hz, 1H), 8.61 (d, J=2.4 Hz, 1H), 7.79 (brs, 1H), 7.46-7.32 (m, 3H), 7.28-7.26 (m, 2H), 5.30 (dd, J=8.0, 4.0 Hz, 1H), 3.15-2.98 (m, 3H), 2.64-2.51 (m, 4H).

The synthesis procedure of Examples (Compounds) 639-641 were similar to the procedure of Example 638.

| Compound | | | Appearance | | MS(m/z) |
|---|---|---|---|---|---|
| No. Method | Activity | Structure and Name | and Yield | ¹H NMR Data | [M + H]⁺ |
| Example 638 Method 118 single unknown stereoisomer | ++++ | 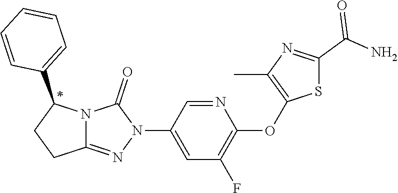 [SMILES: Cc1nc(C(N)=O)sc1Oc1ncc(-n2nc3n(c2=O)[C@H](c2ccccc2)CC3)cc1F] (S)-5-((3-fluoro-5-(3-oxo-5-pheny1-6,7-dihydro-3H-pyrrolo[2,1-c] [1,2,4]triazol-2(5H)-yl)pyridin-2-yl)oxy)-4-methylthiazole-2-carboxamide | white solid, yield: 33.5% | ¹H NMR (400 MHZ, Chloroform-d) δ 8.72 (d, J = 2.4 Hz, 1H), 8.60 (d, J = 2.4 Hz, 1H), 7.58 (brs, 1H), 7.46-7.32 (m, 3H), 7.28-7.26 (m, 2H), 5.29 (dd, J = 8.0, 4.4 Hz, 1H), 3.15-3.01 (m, 2H), 3.01-2.89 (m, 1H), 2.62-2.52 (m, 4H). | 453.4 |
| Example 639 Method 118 single unknown stereoisomer | +++ | 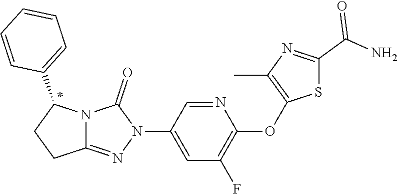 [SMILES: Cc1nc(C(N)=O)sc1Oc1ncc(-n2nc3n(c2=O)[C@@H](c2ccccc2)CC3)cc1F] (R)-5-((3-fluoro-5-(3-oxo-5-phenyl-6,7-dihydro-3H-pyrrolo[2,1-c] [1,2,4]triazol-2(5H)-yl)pyridin-2-yl)oxy)-4-methylthiazole-2-carboxamide | white solid, yield: 33.2% | 1H NMR (400 MHZ, Chloroform-d) δ 8.74 (d, J = 2.4 Hz, 1H), 8.61 (d, J = 2.4 Hz, 1H), 7.79 (brs, 1H), 7.46-7.32 (m, 3H), 7.28-7.26 (m, 2H), 5.30 (dd, J = 8.0, 4.0 Hz, 1H), 3.15-2.98 (m, 3H), 2.64-2.51 (m, 4H). | 453.4 |
| Example 640 Method 118 single unknown stereoisomer | ++++ | 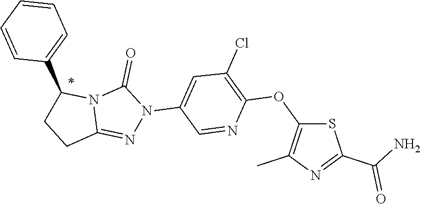 [SMILES: Cc1nc(C(N)=O)sc1Oc1ncc(-n2nc3n(c2=O)[C@H](c2ccccc2)CC3)cc1Cl] (S)-5-((3-chloro-5-(3-oxo-5-phenyl-6,7-dihydro-3H-pyrrolo[2,1-c] [1,2,4]triazol-2(5H)-yl)pyridin-2-yl)oxy)-4-methylthiazole-2-carboxamide | white solid, yield: 11.6% | 1H NMR (400 MHZ, Chloroform-d) δ 8.72 (d, J = 2.4 Hz, 2H), 8.60 (d, J = 2.4 Hz, 2H), 7.46-7.32 (m, 5H), 5.29 (dd, J = 8.0, 4.2 Hz, 1H), 3.19-2.89 (m, 3H), 2.61-2.55 (m, 1H), 2.53 (s, 3H). | 469.2 |
| Example 641 Method 118 single unknown stereoisomer | ++ | 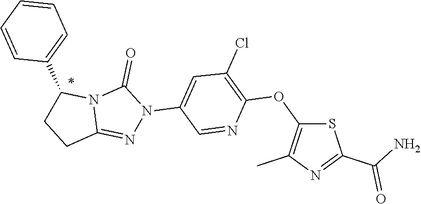 [SMILES: Cc1nc(C(N)=O)sc1Oc1ncc(-n2nc3n(c2=O)[C@@H](c2ccccc2)CC3)cc1Cl] (R)-5-((3-chloro-5-(3-oxo-5-phenyl-6,7-dihydro-3H-pyrrolo[2,1-c] [1,2,4]triazol-2(5H)-yl)pyridin-2-yl)oxy)-4-methylthiazole-2-carboxamide | white solid, yield: 10.9% | 1H NMR (400 MHz, Chloroform-d) δ 8.74 (d, J = 2.4 Hz, 2H), 8.61 (d, J = 2.3 Hz, 2H), 7.46-7.32 (m, 5H), 5.30 (dd, J = 7.9, 4.2 Hz, 1H), 3.15-2.86 (m, 3H), 2.61-2.56 (m, 1H), 2.57 (s, 3H). | 469.2 |

### Example 642: Method 119

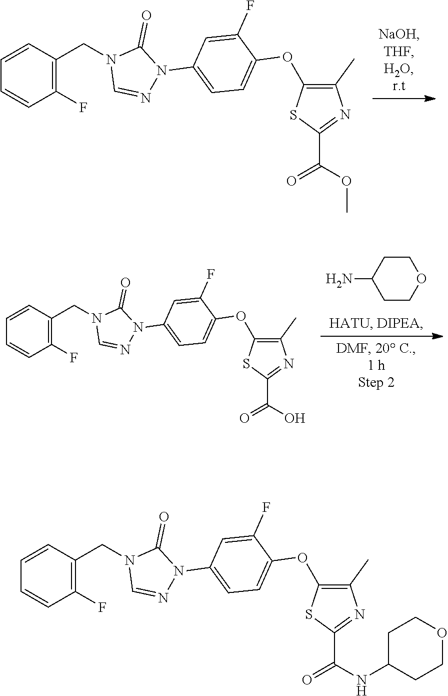 [SMILES: COC(=O)c1nc(C)c(Oc2ccc(-n3ncn(Cc4ccccc4F)c3=O)cc2F)s1 ; Cc1nc(C(=O)O)sc1Oc1ccc(-n2ncn(Cc3ccccc3F)c2=O)cc1F ; NC1CCOCC1 ; Cc1nc(C(=O)NC2CCOCC2)sc1Oc1ccc(-n2ncn(Cc3ccccc3F)c2=O)cc1F]

### Step 1: 5-(2-fluoro-4-(4-(2-fluorobenzyl)-5-oxo-4,5-dihydro-1H-1,2,4-triazol-1-yl)phenoxy)-4-methylthiazole-2-carboxylic Acid

To a solution of methyl 5-[2-fluoro-4-[4-[(2-fluorophenyl)methyl]-5-oxo-1,2,4-triazol-1-yl]phenoxy]-4-methyl-thiazole-2-carboxylate (0.1000 eq, 91 mg, 0.198 mmol) in THF (5 mL), was added NaOH (aq) (2.00 eq, 2.0 mL, 3.95 mmol) at 40° C. The mixture was stirred for 20 min at 40° C. under nitrogen. The reaction was concentrated to dryness. The crude was then adjusted to pH at 4 with 1 N HCl (aq.) and the residue was taken up in EtOAc (50 mL) and the organic layers were washed with 2×20 mL water. The organics were then separated and dried (MgSO₄) before concentration to dryness. The crude mixture was used in the next step reaction without purification. MS (m/z): 445.2 [M+H]⁺.

### Step 2: 5-(2-fluoro-4-(4-(2-fluorobenzyl)-5-oxo-4,5-dihydro-1H-1,2,4-triazol-1-yl) phenoxy)-4-methyl-N-(tetrahydro-2H-pyran-4-yl)thiazole-2-carboxamide

To a solution of 5-[2-fluoro-4-[4-[(2-fluorophenyl)methyl]-5-oxo-1,2,4-triazol-1-yl]phenoxy]-4-methyl-thiazole-2-carboxylic acid (1.00 eq, 70 mg, 0.158 mmol) in DMF (5 mL), were added HATU (1.10 eq, 66 mg, 0.173 mmol), DIPEA (1.50 eq, 30 mg, 0.236 mmol), and Tetrahydro-2H-pyran-4-amine (1.00 eq, 16 mg, 0.158 mmol) at 20° C. The mixture was stirred for 20 min at 20° C. under nitrogen. The reaction mixture was concentrated to dryness and the residue was taken up in EtOAc (50 mL) and the organics were washed with 2×20 mL water and then 1×20 mL saturated brine solution. The organics were then separated and dried (MgSO₄) before concentration to dryness. The crude was then purified by flash column chromatography eluted with 60 ACN in H₂O. The desired fractions were concentrated to dryness in vacuo to afford 5-[2-fluoro-4-[4-[(2-fluorophenyl)methyl]-5-oxo-1,2,4-triazol-1-yl]phenoxy]-4-methyl-N-tetrahydropyran-4-yl-thiazole-2-carboxamide (30 mg, 0.053 mmol, 33.94% yield) as a white solid. MS (m/z): 528.2 [M+H]⁺. ¹H NMR (400 MH z, DMSO-d₆) δ 8.72 (d, J=8.4 Hz, 1H), 8.37 (s, 1H), 7.92 (dd, J=12.4, 2.8 Hz, 1H), 7.73 (dt, J=9.2, 2.0 Hz, 1H), 7.46-7.34 (m, 3H), 7.30-7.18 (m, 2H), 4.96 (s, 2H), 3.96 (ddt, J=14.4, 10.4, 5.2 Hz, 1H), 3.85 (dd, J=11.2, 3.6 Hz, 2H), 3.35 (dt, J=11.36, 6.0 Hz, 2H), 2.31 (s, 3H), 1.77-1.61 (m, 4H).

| Compound | | | Appear- | | |
|---|---|---|---|---|---|
| No. | | | ance and | | MS(m/z) |
| Method | Activity | Structure and Name | Yield | ¹H NMR Data | [M + H]⁺ |
| Example 642 Method 119 | ++++ | 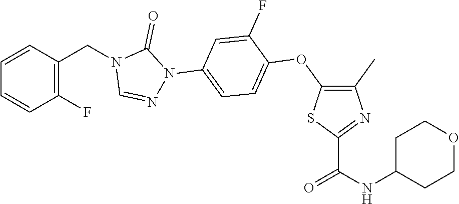 [SMILES: Cc1nc(C(=O)NC2CCOCC2)sc1Oc1ccc(-n2ncn(Cc3ccccc3F)c2=O)cc1F] 5-(2-fluoro-4-(4-(2-fluorobenzyl)-5-oxo-4,5-dihydro-1H-1,2,4-triazol-1-yl) phenoxy)-4-methyl-N-(tetrahydro-2H-pyran-4-yl)thiazole-2-carboxamide | white solid, yield: 33.9% | ¹H NMR (400 MHZ, DMSO-d₆) δ 8.72 d, J = 8.4 Hz, 1H), 8.37 (s, 1H), 7.92 (dd, J = 12.4, 2.8 Hz, 1H), 7.73 (dt, J = 9.2, 2.0 Hz, 1H), 7.46-7.34 (m, 3H), 7.30-7.18 (m, 2H), 4.96 (s, 2H), 3.96 (ddt, J = 14.4, 10.4, 5.2 Hz, 1H), 3.85 (dd, J = 11.2, 3.6 Hz, 2H), 3.35 (dt, J = 11.6, 6.0 Hz, 2H), 2.31 (s, 3H), 1.77-1.61 (m, 4H). | 528.2 |

### Example 643: Method 120

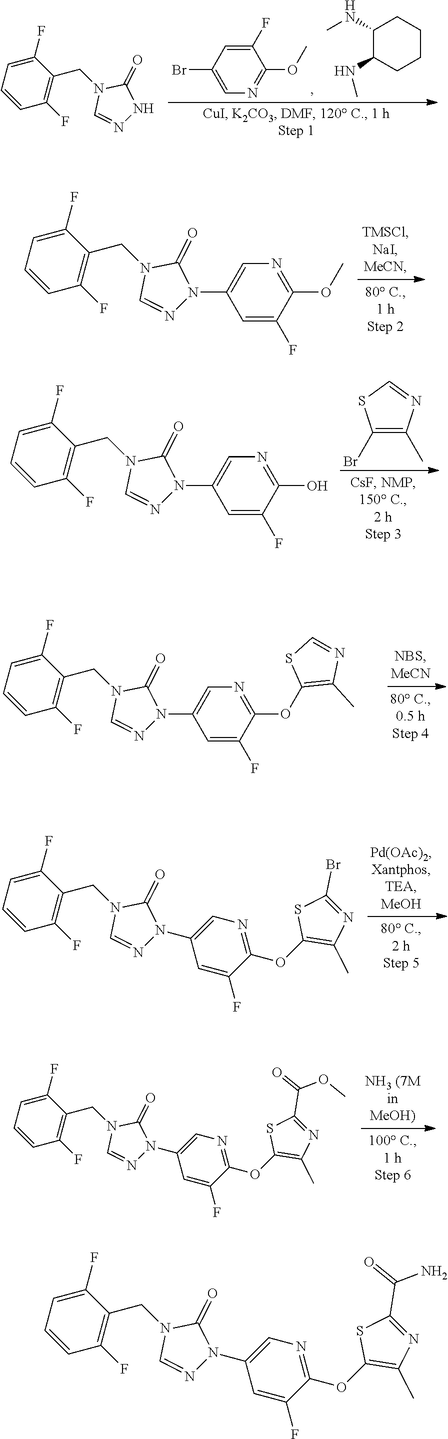

### Step 1: 4-[(2,6-difluorophenyl)methyl]-2-(5-fluoro-6-methoxy-3-pyridyl)-1,2,4-triazol-3-one

To a solution of 4-[(2,6-difluorophenyl)methyl]-1H-1,2,4-triazol-5-one (1.00 eq, 6000 mg, 28.4 mmol) in DMF (60 mL), was added CuI (0.500 eq, 2699 mg, 14.2 mmol), K₂CO₃ (2.00 eq, 7842 mg, 56.8 mmol), (1R,2R)—N¹,N²-dimethylcyclohexane-1,2-diamine (1.00 eq, 4035 mg, 28.4 mmol), and 5-bromo-3-fluoro-2-methoxy-pyridine (1.00 eq, 5853 mg, 28.4 mmol). The mixture was stirred for 1 h at 120° C. under nitrogen atmosphere. The suspension was filtered through a pad of Celite or silica gel and the pad or filter cake was washed with EA (100 mL×3). The combined filtrates were washed with brine (200 mL×3). The organics were then separated and dried (MgSO₄) before concentration to dryness. The crude was then triturated with MeOH/PE (5:2). The mixture was filtered and the filter cake was washed with 100 mL of PE, dried in vacuum to give 4-[(2,6-difluorophenyl)methyl]-2-(5-fluoro-6-methoxy-3-pyridyl)-1,2,4-triazol-3-one (4000 mg, 11.3 mmol, 39.8% yield) as a white solid. MS (m/z): 337.1 [M+H]⁺.

### Step 2: 4-[(2,6-difluorophenyl)methyl]-2-(5-fluoro-6-hydroxy-3-pyridyl)-1,2,4-triazol-3-one

To a solution of NaI (3.00 eq, 5317 mg, 35.7 mmol) in MeCN (200 mL), was added chlorotrimethylsilane (3.00 eq, 3890 mg, 35.7 mmol), and 4-[(2,6-difluorophenyl)methyl]-2-(5-fluoro-6-methoxy-3-pyridyl)-1,2,4-triazol-3-one (1.00 eq, 4000 mg, 11.9 mmol). The mixture was stirred for 1 h at 80° C. under nitrogen atmosphere. The reaction was concentrated to dryness and the residue was taken up in EA (300 mL) and the organics were washed with 2×100 mL water and then 1×100 mL saturated brine solution. The organics were then separated and dried (MgSO₄) before concentration to dryness. The crude was then triturated with MeOH to afford 4-[(2,6-difluorophenyl)methyl]-2-(5-fluoro-6-hydroxy-3-pyridyl)-1,2,4-triazol-3-one (4000 mg, 11.8 mmol, 99.2% yield) as a brown solid. MS (m/z): 323.1 [M+H]⁺.

### Step 3: 4-[(2,6-difluorophenyl)methyl]-2-[5-fluoro-6-(4-methylthiazol-5-yl)oxy-3-pyridyl]-1,2,4-triazol-3-one

To a solution of 4-[(2,6-difluorophenyl)methyl]-2-(5-fluoro-6-hydroxy-3-pyridyl)-1,2,4-triazol-3-one (1.00 eq, 4000 mg, 12.4 mmol) in NMP (100 mL) was added 5-bromo-4-methylthiazole (1.10 eq, 2431 mg, 13.7 mmol), and CsF (3.00 eq, 5660 mg, 37.2 mmol). The mixture was stirred for 2 h at 150° C. under nitrogen atmosphere. The reaction was concentrated to dryness and the residue was taken up in EA (500 mL) and the organics were washed with 2×100 mL water and then 1×100 mL saturated brine solution. The organics were then separated and dried (MgSO₄) before concentration to dryness. The crude was then purified by flash column chromatography eluted with 100% EA in PE. The desired fractions were concentrated to dryness in vacuo to afford 4-[(2,6-difluorophenyl)methyl]-2-[5-fluoro-6-(4-methylthiazol-5-yl)oxy-3-pyridyl]-1,2,4-triazol-3-one (1300 mg, 2.94 mmol, 23.72% yield) as a yellow solid. MS (m/z): 420.1 [M+H]⁺. ¹H NMR (400 MHz, DMSO-d₆) δ 8.76 (s, 1H), 8.49 (d, J=2.0 Hz, 1H), 8.41 (s, 1H), 8.31 (dd, J=11.2, 2.0 Hz, 1H), 7.49 (ddd, J=15.2, 8.4, 6.4 Hz, 1H), 7.16 (t, J=8.0 Hz, 2H), 4.99 (s, 2H), 2.26 (s, 3H).

### Step 4: 2-[6-(2-bromo-4-methyl-thiazol-5-yl)oxy-5-fluoro-3-pyridyl]-4-[(2,6-difluorophenyl)methyl]-1,2,4-triazol-3-one

To a solution of 4-[(2,6-difluorophenyl)methyl]-2-[5-fluoro-6-(4-methylthiazol-5-yl)oxy-3-pyridyl]-1,2,4-triazol-3-one (1.00 eq, 1300 mg, 3.10 mmol) in MeCN (20 mL), was added NBS (1.10 eq, 607 mg, 3.41 mmol). The mixture was stirred for 30 min at 80° C. under nitrogen atmosphere. The reaction was quenched with Na₂S₂O₃(aq.) and the residue was taken up in EA (100 mL) and the organics were washed with 2×100 mL water and then 1×100 mL saturated brine solution. The organics were then separated and dried (MgSO₄) before concentration to dryness. The residues was triturated with MeOH to afford 2-[6-(2-bromo-4-methyl-thiazol-5-yl)oxy-5-fluoro-3-pyridyl]-4-[(2,6-difluorophenyl)methyl]-1,2,4-triazol-3-one (1100 mg, 2.14 mmol, 69.08% yield) as a yellow solid. MS (m/z): 498.2,500.2 [M+H]⁺. H NMR (400 MHz, DMSO-d₆) δ 8.53 (d, J=2.0 Hz, 1H), 8.42 (s, 1H), 8.34 (dd, J=11.2, 2.0 Hz, 1H), 7.49 (tt, J=8.4, 6.8 Hz, 1H), 7.16 (t, J=8.0 Hz, 2H), 4.99 (s, 2H), 2.28 (s, 3H).

### Step 5: methyl 5-[[5-[4-[(2,6-difluorophenyl)methyl]-5-oxo-1,2,4-triazol-1-yl]-3-fluoro-2-pyridyl]oxy]-4-methyl-thiazole-2-carboxylate

To a solution of 2-[6-(2-bromo-4-methyl-thiazol-5-yl)oxy-5-fluoro-3-pyridyl]-4-[(2,6-difluorophenyl)methyl]-1,2,4-triazol-3-one (1.00 eq, 1000 mg, 2.01 mmol) in Methanol (50 mL), was added Pd(OAc)₂ (0.500 eq, 225 mg, 1.00 mmol), Xantphos (0.800 eq, 929 mg, 1.61 mmol), and TEA (3.00 eq, 0.84 mL, 6.02 mmol). The mixture was stirred for 2 h at 80° C. under CO atmosphere. The mixture was diluted with DCM (200 mL). The suspension was filtered and the filter cake was washed with MeOH. The combined filtrates were concentrated to dryness to give the product methyl 5-[[5-[4-[(2,6-difluorophenyl)methyl]-5-oxo-1,2,4-triazol-1-yl]-3-fluoro-2-pyridyl]oxy]-4-met hyl-thiazole-2-carboxylate (650 mg, 1.28 mmol, 63.77% yield) as a green solid. MS (m/z): 478.2 [M+H]⁺. ¹H NMR (400 MHz, DMSO-d₆) δ 8.58 (d, J=2.4 Hz, 1H), 8.43 (s, 1H), 8.37 (dd, J=11.2, 2.0 Hz, 1H), 7.55-7.43 (m, 1H), 7.16 (t, J=8.0 Hz, 2H), 5.00 (s, 2H), 3.90 (s, 3H), 2.41 (s, 3H).

### Step 6: 5-[[5-[4-[(2,6-difluorophenyl)methyl]-5-oxo-1,2,4-triazol-1-yl]-3-fluoro-2-pyridyl]oxy]-4-methyl-thiazole-2-carboxamide

To a solution of methyl 5-[[5-[4-[(2,6-difluorophenyl)methyl]-5-oxo-1,2,4-triazol-1-yl]-3-fluoro-2-pyridyl]oxy]-4-methyl-thiazole-2-carboxylate (1.00 eq, 89 mg, 0.187 mmol) in Methanol (5 mL), was added NH₃ (7 M in MeOH) (10 mL). The mixture was stirred for 1 h at 100° C. under nitrogen. The reaction mixture was concentrated to dryness. The crude was then triturated with MeOH and the solid 5-[[5-[4-[(2,6-difluorophenyl)methyl]-5-oxo-1,2,4-triazol-1-yl]-3-fluoro-2-pyridyl]oxy]-4-met hyl-thiazole-2-carboxamide (56 mg, 0.113 mmol, 60.34% yield) was obtained as a yellow solid. MS (m/z): 463.2 [M+H]⁺. ¹H NMR (400 MHz, DMSO-d₆) δ 8.54 (d, J=2.4 Hz, 1H), 8.42 (s, 1H), 8.35 (dd, J=11.2, 2.4 Hz, 1H), 8.06 (brs, 1H), 7.76 (brs, 1H), 7.55-7.43 (m, 1H), 7.16 (t, J=8.0 Hz, 2H), 5.00 (s, 2H), 2.34 (s, 3H).

The synthesis procedure of Examples (Compounds) 644-647 were similar to the procedure of Example 643.

| Compound | | | Appearance | | MS(m/z) |
|---|---|---|---|---|---|
| No. Method | Activity | Structure and Name | and Yield | ¹H NMR Data | [M + H]⁺ |
| Example 643 Method 120 | ++++ | 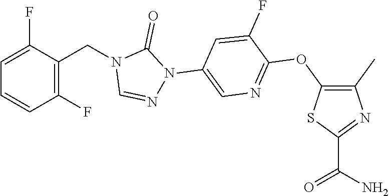 [SMILES: Cc1nc(C(N)=O)sc1Oc1ncc(-n2ncn(Cc3c(F)cccc3F)c2=O)cc1F] 5-((5-(4-(2,6-difluorobenzyl)-5-oxo-4,5-dihydro-1H-1,2,4-triazol- 1-yl)-3-fluoropyridin-2-yl)oxy)-4-methylthiazole-2-carboxamide | yellow solid, yield: 60.1% | ¹H NMR (400 MHZ, DMSO-d₆) δ 8.54 (d, J = 2.4 Hz, 1H), 8.42 (s, 1H), 8.35 (dd, J = 11.2, 2.4 Hz, 1H), 8.06 (brs, 1H), 7.76 (brs, 1H), 7.55- 7.43 (m, 1H), 7.16 (t, J = 8.0 Hz, 2H), 5.00 (s, 2H), 2.34 (s, 3H). | 463.2 |
| Example 644 Method 120 | ++++ | 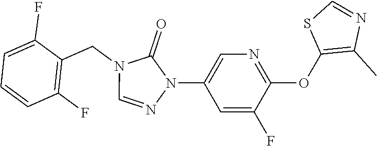 [SMILES: Cc1ncsc1Oc1ncc(-n2ncn(Cc3c(F)cccc3F)c2=O)cc1F] 4-(2,6-difluorobenzyl)-2-(5-fluoro-6-((4-methylthiazol-5-yl)oxy) pyridin-3-y1)-2,4-dihydro-3H-1,2,4-triazol-3-one | white solid, yield: 23.7%. | ¹H NMR (400 MHZ, DMSO-d₆) δ 8.76 (s, 1H), 8.49 (d, J = 2.0 Hz, 1H), 8.41 (s, 1H), 8.31 (dd, J = 11.2, 2.0 Hz, 1H), 7.49 (ddd, J = 15.2, 8.4, 6.4 Hz, 1H), 7.16 (t, J = 8.0 Hz, 2H), 4.99 (s, 2H), 2.26 (s, 3H). | 420.2 |
| Example 645 Method 120 | ++++ | 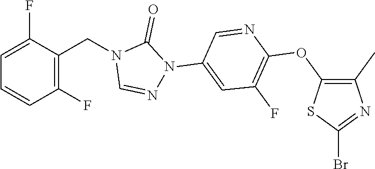 [SMILES: Cc1nc(Br)sc1Oc1ncc(-n2ncn(Cc3c(F)cccc3F)c2=O)cc1F] 2-(6-((2-bromo-4-methylthiazol-5-y1)oxy)-5-fluoropyridin-3-yl)- 4-(2,6-difluorobenzyl)-2,4-dihydro-3H-1,2,4-triazol-3-one | white solid, yield: 23.7% | ¹H NMR (400 MHZ, DMSO-d₆) δ 8.53 (d, J = 2.0 Hz, 1H), 8.42 (s, 1H), 8.34 (dd, J = 11.2, 2.0 Hz, 1H), 7.49 (tt, J = 8.4, 6.8 Hz, 1H), 7.16 (t, J = 8.0 Hz, 2H), 4.99 (s, 2H), 2.28 (s, 3H). | 498.2, 500.2 |
| Example 646 Method 120 | ++++ | 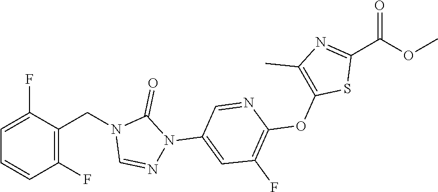 [SMILES: COC(=O)c1nc(C)c(Oc2ncc(-n3ncn(Cc4c(F)cccc4F)c3=O)cc2F)s1] methyl 5-((5-(4-(2,6-difluorobenzyl)-5-oxo-4,5-dihydro-1H-1,2,4-triazol- 1-yl)-3-fluoropyridin-2-yl)oxy)-4-methylthiazole-2-carboxylate | brown solid, yield: 63.7% | ¹H NMR (400 MHZ, DMSO-d₆) δ 8.58 (d, J = 2.4 Hz, 1H), 8.43 (s, 1H), 8.37 (dd, J = 11.2, 2.0 Hz, 1H), 7.55-7.43 (m, 1H), 7.16 (t, J = 8.0 Hz, 2H), 5.00 (s, 2H), 3.90 (s, 3H), 2.41 (s, 3H). | 478.2 |
| Example 647 Method 120 | ++++ | 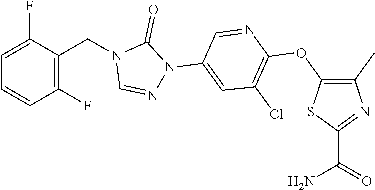 [SMILES: Cc1nc(C(N)=O)sc1Oc1ncc(-n2ncn(Cc3c(F)cccc3F)c2=O)cc1Cl] 5-((3-chloro-5-(4-(2,6-difluorobenzy1)-5-oxo-4,5-dihydro-1H- 1,2,4-triazol-1-yl)pyridin-2-yl)oxy)-4-methylthiazole-2-carboxamide | white solid, yield: 12.7% | ¹H NMR (400 MHZ, DMSO-d₆) δ 8.66 (d, J = 2.4 Hz, 1H), 8.53 (d, J = 2.4 Hz, 1H), 8.44-8.40 (m, 1H), 8.08-8.03 (m, 1H), 7.78-7.73 (m, 1H), 7.51-7.47 (m, 1H), 7.16 (t, J = 8.0 Hz, 2H), 4.99 (s, 2H), 2.36 (s, 3H). | 479.2 |

### Example 648: Method 121

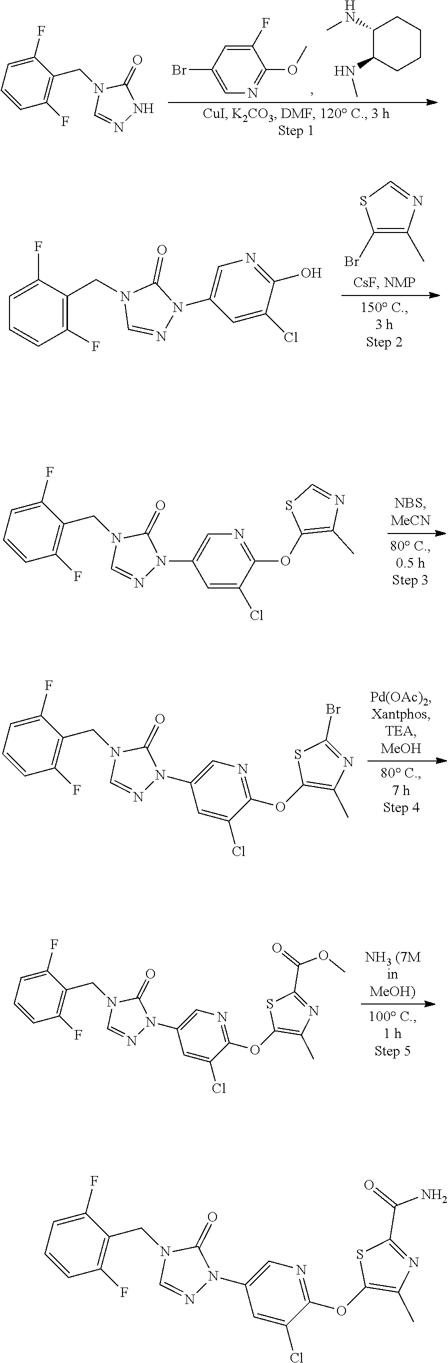

### Step 1: 2-(5-chloro-6-hydroxy-3-pyridyl)-4-[(2,6-difluorophenyl)methyl]-1,2,4-triazol-3-one

To a solution of 4-[(2,6-difluorophenyl)methyl]-1H-1,2,4-triazol-5-one (1.00 eq, 6000 mg, 28.4 mmol) in DMF (200 mL), was added 5-bromo-3-chloro-pyridin-2-ol (1.20 eq, 7107 mg, 34.1 mmol), CuI (1.00 eq, 5398 mg, 28.4 mmol), DMG (1.00 eq, 2927 mg, 28.4 mmol), and Cs₂CO₃ (3.00 eq, 27703 mg, 85.2 mmol). The mixture was stirred for 3 h at 120° C. under nitrogen atmosphere. The reaction mixture was concentrated to dryness and the residue was taken up in EA (200 mL) and the organics were washed with 2×50 mL water and then 1×50 mL saturated brine solution. The water phase was adjusted to pH 5 by HCl (aq., 1 M) and extracted with EA (200 mL). The organics were then separated and dried (MgSO₄) before concentration to dryness. The crude was then triturated with EA/MeOH to afford 2-(5-chloro-6-hydroxy-3-pyridyl)-4-[(2,6-difluorophenyl)methyl]-1,2,4-triazol-3-one (2600 mg, 7.68 mmol, 27.02% yield) as a yellow solid. MS (m/z): 339.1 [M+H]⁺.

### Step 2: 2-[5-chloro-6-(4-methylthiazol-5-yl)oxy-3-pyridyl]-4-[(2,6-difluorophenyl)methyl]-1,2,4-triazol-3-one

To a solution of 2-(5-chloro-6-hydroxy-3-pyridyl)-4-[(2,6-difluorophenyl)methyl]-1,2,4-triazol-3-one (1.00 eq, 2600 mg, 7.67 mmol) in NMP(150 mL), was added 5-Bromo-4-methylthiazole (1.20 eq, 1639 mg, 9.17 mmol), and CsF (2.00 eq, 2316 mg, 15.2 mmol). The mixture was stirred for 3 h at 150° C. under nitrogen atmosphere. The reaction mixture was concentrated to dryness and the residue was taken up in EA (200 mL) and the organics were washed with 2×50 mL water and then 1×50 mL saturated brine solution. The organics were then separated and dried (MgSO₄) before concentration to dryness. The crude was then purified by flash column chromatography eluted with 30% EA in PE. The desired fractions were concentrated to dryness in vacuo to afford 2-[5-chloro-6-(4-methylthiazol-5-yl)oxy-3-pyridyl]-4-[(2,6-difluorophenyl)methyl]-1,2,4-triaz ol-3-one (700 mg, 1.53 mmol, 20.01% yield). MS (m/z): 436.1 [M+H]⁺. ¹H NMR (400 MHz, Chloroform-d) δ 8.72 (d, J=2.4 Hz, 1H), 8.53 (d, J=2.4 Hz, 1H), 8.49 (s, 1H), 7.62 (s, 1H), 7.38 (tt, J=8.4, 6.5 Hz, 1H), 7.06-6.92 (m, 2H), 4.98 (t, J=1.2 Hz, 2H), 2.42 (s, 3H).

### Step 3: 2-[6-(2-bromo-4-methyl-thiazol-5-yl)oxy-5-chloro-3-pyridyl]-4-[(2,6-difluorophenyl)methyl]-1,2,4-triazol-3-one

To a solution of 2-[5-chloro-6-(4-methylthiazol-5-yl)oxy-3-pyridyl]-4-[(2,6-difluorophenyl) methyl]-1,2,4-triazol-3-one (1.00 eq, 300 mg, 0.688 mmol) in MeCN (20 mL), was added NBS (1.20 eq, 146 mg, 0.826 mmol). The mixture was stirred for 30 min at 80° C. under nitrogen atmosphere. The reaction mixture was concentrated to dryness and the residue was taken up in EA(50 mL) and the organics were washed with 2×20 mL water and then 1×20 mL saturated brine solution. The organics were then separated and dried (MgSO₄) before concentration to dryness. The desired fractions were concentrated to dryness in vacuo to afford 2-[6-(2-bromo-4-methyl-thiazol-5-yl)oxy-5-chloro-3-pyridyl]-4-[(2,6-difluorophenyl)methyl]-1,2,4-triazol-3-one (300 mg, 0.53 mmol, 76.20% yield). The product was used in the next step without purification. MS (m/z): 514.2 [M+H]⁺.

### Step 4: 5-[[3-chloro-5-[4-[(2,6-difluorophenyl)methyl]-5-oxo-1,2,4-triazol-1-yl]-2-pyridyl]oxy]-4-methyl-thiazole-2-carboxylate

To a solution of 2-[6-(2-bromo-4-methyl-thiazol-5-yl)oxy-5-chloro-3-pyridyl]-4-[(2,6-difluorophenyl)methyl]-1,2,4-triazol-3-one (1.00 eq, 250 mg, 0.486 mmol) in methanol (10 mL), were added Pd(OAc)₂ (0.200 eq, 22 mg, 0.0971 mmol), xantphos (0.400 eq, 112 mg, 0.194 mmol), and TEA (2.00 eq, 0.14 mL, 0.971 mmol). The mixture was stirred for 7 h at 80° C. under CO atmosphere. The reaction mixture was concentrated to dryness and the residue was taken up in EA (50 mL) and the organics were washed with 2×30 mL water and then 1×30 mL saturated brine solution. The organics were then separated and dried (MgSO₄) before concentration to dryness. The crude was then purified by flash column chromatography eluted 50% EA in PE. The desired fractions were concentrated to dryness in vacuo to afford 5-[[3-chloro-5-[4-[(2,6-difluorophenyl)methyl]-5-oxo-1,2,4-triazol-1-yl]-2-pyridyl]oxy]-4-met hyl-thiazole-2-carboxylate (150 mg, 0.292 mmol, 60.03% yield) as a yellow solid. MS (m/z): 494.1 [M+H]⁺. ¹H NMR (400 MHz, Chloroform-d) δ 8.78 (d, J=2.4 Hz, 1H), 8.60 (d, J=2.4 Hz, 1H), 7.64 (s, 1H), 7.39 (tt, J=8.4, 6.4 Hz, 1H), 7.00 (t, J=8.0 Hz, 2H), 4.99 (s, 2H), 4.00 (s, 3H), 2.54 (s, 3H).

### Step 5: 5-[[3-chloro-5-[4-[(2,6-difluorophenyl)methyl]-5-oxo-1,2,4-triazol-1-yl]-2-pyridyl]oxy]-4-methyl-thiazole-2-carboxamide

A solution of methyl 5-[[3-chloro-5-[4-[(2,6-difluorophenyl)methyl]-5-oxo-1,2,4-triazol-1-yl]-2-pyridyl]oxy]-4-methyl-thiazole-2-carboxylate (1.00 eq, 100 mg, 0.202 mmol) in NH₃ (7M in MeOH)(5.0 mL) was stirred for 1 h at 100° C. The reaction mixture was concentrated to dryness and the residue was taken up in EA(50 mL) and the organics were washed with 2×20 mL water and then 1×20 mL saturated brine solution. The organics were then separated and dried (MgSO₄) before concentration to dryness. The crude was then purified by flash column chromatography eluted with 50% ACN in ACN/H₂O. The desired fractions were concentrated to dryness in vacuo to afford 5-[[3-chloro-5-[4-[(2,6-difluorophenyl)methyl]-5-oxo-1,2,4-triazol-1-yl]-2-pyridyl]oxy]-4-methyl-thiazole-2-carboxamide (13 mg, 0.0244 mmol, 12.07% yield) as a white solid. MS (m/z): 479.2 [M+H]⁺. ¹H NMR (400 MHz, DMSO-d₆) δ 8.66 (d, J=2.4 Hz, 1H), 8.53 (d, J=2.4 Hz, 1H), 8.44-8.40 (m, 1H), 8.08-8.03 (m, 1H), 7.78-7.73 (m, 1H), 7.51-7.47 (m, 1H), 7.16 (t, J=8.0 Hz, 2H), 4.99 (s, 2H), 2.36 (s, 3H).

The synthesis procedure of Example (Compound) 649 was similar to the procedure of Example 648.

| Compound | | | Appearance | | MS(m/z) |
|---|---|---|---|---|---|
| No. Method | Activity | Structure and Name | and Yield | ¹H NMR Data | [M + H]⁺ |
| Example 648 Method 121 | ++++ | 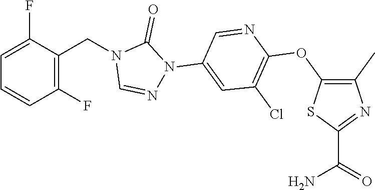 [SMILES: Cc1nc(C(N)=O)sc1Oc1ncc(-n2ncn(Cc3c(F)cccc3F)c2=O)cc1Cl] 5-((3-chloro-5-(4-(2,6-difluorobenzyl)-5-oxo-4,5-dihydro-1H- 1,2,4-triazol-1-yl)pyridin-2-yl)oxy)-4-methylthiazole-2-carboxamide | white solid, yield: 12.7% | ¹H NMR (400 MHZ, DMSO-d₆) δ 8.66 (d, J = 2.4 Hz, 1H), 8.53 (d, J = 2.4 Hz, 1H), 8.44-8.40 (m, 1H), 8.08-8.03 (m, 1H), 7.78-7.73 (m, 1H), 7.51-7.47 (m, 1H), 7.16 (t, J = 8.0 Hz, 2H), 4.99 (s, 2H), 2.36 (s, 3H). | 479.2 |
| Example 649 Method 121 | ++++ | 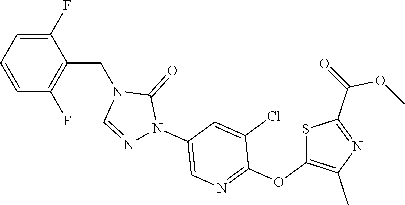 [SMILES: COC(=O)c1nc(C)c(Oc2ncc(-n3ncn(Cc4c(F)cccc4F)c3=O)cc2Cl)s1] methyl 5-((3-chloro-5-(4-(2,6-difluorobenzyl)-5-oxo-4,5-dihydro-1H- 1,2,4-triazol-1-yl)pyridin-2-yl)oxy)-4-methylthiazole-2-carboxylate | yellow solid, yield: 60.3% | ¹H NMR (400 MHZ, Chloroform-d) δ 8.78 (d, J = 2.4 Hz, 1H), 8.60 (d, J = 2.4 Hz, 1H), 7.64 (s, 1H), 7.39 (tt, J = 8.4, 6.4 Hz, 1H), 7.00 (t, J = 8.0 Hz, 2H), 4.99 (s, 2H), 4.00 (s, 3H), 2.54 (s, 3H). | 494.2 |

### Example 650: Method 122

### 2-(5-(4-(4-(2,6-difluorobenzyl)-5-oxo-4,5-dihydro-1H-1,2,4-triazol-1-yl)-2-fluorophenoxy)-4-methylisoxazol-3-yl)acetonitrile

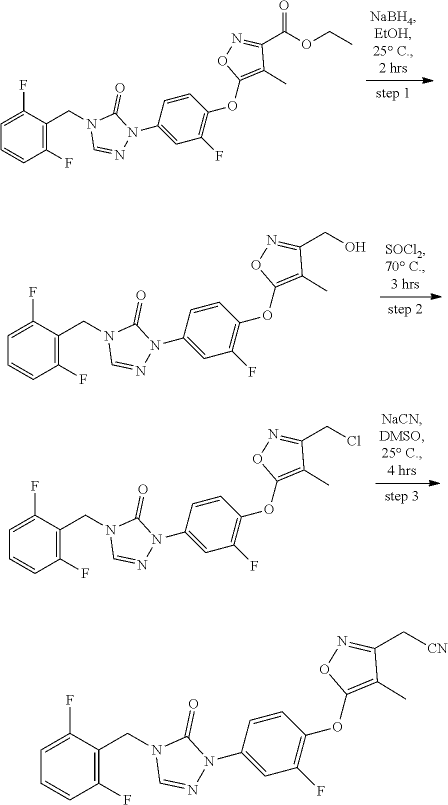 [SMILES: CCOC(=O)c1noc(Oc2ccc(-n3ncn(Cc4c(F)cccc4F)c3=O)cc2F)c1C ; Cc1c(CO)noc1Oc1ccc(-n2ncn(Cc3c(F)cccc3F)c2=O)cc1F ; Cc1c(CCl)noc1Oc1ccc(-n2ncn(Cc3c(F)cccc3F)c2=O)cc1F ; Cc1c(CC#N)noc1Oc1ccc(-n2ncn(Cc3c(F)cccc3F)c2=O)cc1F]

### Step 1: 4-(2,6-difluorobenzyl)-2-(3-fluoro-4-((3-(hydroxymethyl)-4-methylisoxazol-5-yl)oxy)phenyl)-2,4-dihydro-3H-1,2,4-triazol-3-one

To a stirred solution of ethyl 5-(4-(4-(2,6-difluorobenzyl)-5-oxo-4,5-dihydro-1H-1,2,4-triazol-1-yl)-2-fluorophenoxy)-4-methylisoxazole-3-carboxylate (200 mg, 0.42 mmol) in ethanol (10 mL) was added sodium borohydride (31.9 mg, 0.84 mmol) at 25° C. The reaction mixture was stirred at 25° C. for 2 h. The mixture was diluted with water (10 mL). The resulted mixture was extracted with EtOAc (3×80 mL). The combined organic layers were washed with brine (100 mL), dried over anhydrous Na₂SO₄, filtered, and the filtrate was concentrated under reduced pressure. The residue was purified by prep-TLC (PE/EA=1:1) to afford 4-(2,6-difluorobenzyl)-2-(3-fluoro-4-((3-(hydroxymethyl)-4-methylisoxazol-5-yl)oxy)phenyl)-2,4-dihydro-3H-1,2,4-triazol-3-one (160 mg, 83.4% yield) as an off-white solid. MS (m/z): 433.0 [M+H]⁺.

### Step 2: 2-(4-((3-(chloromethyl)-4-methylisoxazol-5-yl)oxy)-3-fluorophenyl)-4-(2,6-difluorobenzyl)-2,4-dihydro-3H-1,2,4-triazol-3-one

The solution of methyl 1-(4-(3,5-dimethyl-1H-1,2,4-triazol-1-yl)-5-fluoropyrimidin-2-yl) piperidine-4-carboxylate (50 mg, 0.11 mmol) in SOCl₂ (2 mL) was stirred at 70° C. for 3 h. The mixture was adjusted to PH=9 with 2N NaHCO₃. The resulted mixture was extracted with EtOAc (3×5 mL). The combined organic layers were washed with brine (10 mL), dried over anhydrous Na₂SO₄, and after filtration, the filtrate was concentrated under reduced pressure. The residue was purified by prep-TLC (PE/EA=3:1) to afford 2-(4-((3-(chloromethyl)-4-methylisoxazol-5-yl)oxy)-3-fluorophenyl)-4-(2,6-difluorobenzyl)-2,4-dihydro-3H-1,2,4-triazol-3-one (40 mg, 72.9% yield) as a yellow oil. LC-MS (m/z) 451.1 [M+H]⁺.

### Step 3: 2-(5-(4-(4-(2,6-difluorobenzyl)-5-oxo-4,5-dihydro-1H-1,2,4-triazol-1-yl)-2-fluorophenoxy)-4-methylisoxazol-3-yl)acetonitrile

To a stirred solution of 2-(4-((3-(chloromethyl)-4-methylisoxazol-5-yl)oxy)-3-fluorophenyl)-4-(2,6-difluorobenzyl)-2,4-dihydro-3H-1,2,4-triazol-3-one (80 mg, 0.17 mmol) in DMSO (50 mL) was added NaCN (10.4 mg, 0.21 mmol). The mixture was stirred at 25° C. for 4 hours. The resulted mixture was diluted with water (4 mL). The resulted mixture was extracted with EtOAc (3×2 mL). The combined organic layers were washed with brine (4 mL), dried over anhydrous Na₂SO₄, and after filtration, the filtrate was concentrated under reduced pressure. The residue was purified by prep-TLC (PE/EA=1:1) to afford the desired product (18 mg, 22.8% yield) as an off-white solid. LC-MS (m/z): 442.1 [M+H]⁺. ¹H NMR (400 MHz, DMSO-d6) δ 7.98 (dd, J=12.0, 2.4 Hz, 1H), 7.5-7.83 (d, J=8.8 Hz, 1H), 7.62 (s, 1H), 7.41-7.37 (m, 1H), 7.24 (d, J=8.8 Hz, 1H), 7.01 (t, J=8.0 Hz, 1H), 5.00 (s, 2H), 3.72 (s, 2H), 1.97 (s, 3H).

| Compound | | | Appearance | | MS(m/z) |
|---|---|---|---|---|---|
| No. Method | Activity | Structure and Name | and Yield | ¹H NMR Data | [M + H]⁺ |
| Example 650 Method 122 | ++++ | 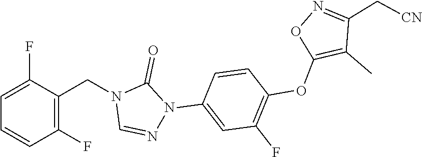 [SMILES: Cc1c(CC#N)noc1Oc1ccc(-n2ncn(Cc3c(F)cccc3F)c2=O)cc1F] 2-(5-(4-(4-(2,6-difluorobenzyl)-5-oxo-4,5-dihydro-1H-1,2,4- triazol-1-yl)-2-fluorophenoxy)-4-methylisoxazol-3-yl)acetonitrile | off-white solid, yield: 22.8% | ¹H NMR (400 MHZ, DMSO-d₆) δ 7.98 (dd, J = 12.0, 2.4 Hz, 1H), 7.5-7.83 (d, J = 8.8 Hz, 1H), 7.62 (s, 1H), 7.41-7.37 (m, 2H), 7.24 (d, J = 8.8 Hz, 1H), 7.01 (t, J = 8.0 Hz, 1H), 5.00 (s, 2H), 3.72 (s, 2H), 1.97 (s, 3H). | 442.1 |

### Example 651: Method 123

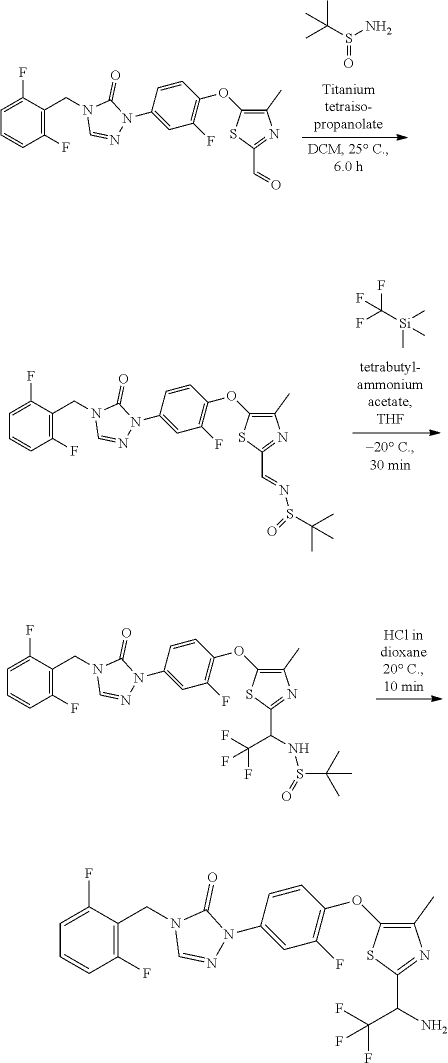

### Step 1: (E)-N-((5-(4-(4-(2,6-difluorobenzyl)-5-oxo-4,5-dihydro-1H-1,2,4-triazol-1-yl)-2-fluorophenoxy)-4-methylthiazol-2-yl)methylene)-2-methylpropane-2-sulfinamide

To a solution of 5-[4-[4-[(2,6-difluorophenyl)methyl]-5-oxo-1,2,4-triazol-1-yl]-2-fluoro-phenoxy]-4-methyl-thiazole-2-carbaldehyde (1.00 eq, 300 mg, 0.672 mmol) in DCM (4 mL), was added titanium tetraisopropanolate (5.00 eq, 954 mg, 3.36 mmol). The mixture was stirred for 10 min at 25° C. under nitrogen. Tert-butanesulfinamide (3.00 eq, 244 mg, 2.02 mmol) was added to the solution. The mixture was stirred at 25° C. for 6 h under nitrogen. The reaction mixture was concentrated to dryness and the residue was taken up in EtOAc (50 mL) and the organics were washed with 2×20 mL water and then 1×20 mL saturated brine solution. The organics were then separated and dried (MgSO₄) before concentration to dryness. The crude was then purified by flash column chromatography eluted with 70% EtOAc in PE. The desired fractions were concentrated to dryness in vacuo to afford (NE)-N-[[5-[4-[4-[(2,6-difluorophenyl)methyl]-5-oxo-1,2,4-triazol-1-yl]-2-fluoro-phenoxy]-4-methyl-thiazol-2-yl]methylene]-2-methyl-propane-2-sulfinamide (230 mg, 62.27% yield) as a yellow oil. MS (m/z): 550.2 [M+H]⁺.

### Step 2: N-(1-(5-(4-(4-(2,6-difluorobenzyl)-5-oxo-4,5-dihydro-1H-1,2,4-triazol-1-yl)-2-fluorophenoxy)-4-methylthiazol-2-yl)-2,2,2-trifluoroethyl)-2-methylpropane-2-sulfinamide

To a solution of (NE)-N-[[5-[4-[4-[(2,6-difluorophenyl)methyl]-5-oxo-1,2,4-triazol-1-yl]-2-fluoro-phenoxy]-4-methyl-thiazol-2-yl]methylene]-2-methyl-propane-2-sulfinamide (1.00 eq, 200 mg, 0.364 mmol) in THF (15 mL), were added trimethyl(trifluoromethyl)silane (2.00 eq, 103 mg, 0.728 mmol), and tetrabutylammonium acetate (1.10 eq, 114 mg, 0.400 mmol). The mixture was stirred for 30 min at −20° C. under nitrogen. The reaction mixture was concentrated to dryness and the residue was taken up in EA (50 mL) and the organics were washed with 2×20 mL water and then 1×20 mL saturated brine solution. The organics were then separated and dried (MgSO₄) before concentration to dryness. The crude was then purified by flash column chromatography eluted with 60% ACN in ACN/H₂O. The desired fractions were concentrated to dryness in vacuo to afford N-[1-[5-[4-[4-[(2,6-difluorophenyl)methyl]-5-oxo-1,2,4-triazol-1-yl]-2-fluoro-phenoxy]-4-met hyl-thiazol-2-yl]-2,2,2-trifluoro-ethyl]-2-methyl-propane-2-sulfinamide (100 mg, 0.161 mmol, 44.35% yield) as a crude oil. MS (m/z): 620.2 [M+H]⁺.

### Step 3: 2-(4-((2-(1-amino-2,2,2-trifluoroethyl)-4-methylthiazol-5-yl)oxy)-3-fluorophenyl)-4-(2,6-difluorobenzyl)-2,4-dihydro-3H-1,2,4-triazol-3-one

To a solution of N-[1-[5-[4-[4-[(2,6-difluorophenyl)methyl]-5-oxo-1,2,4-triazol-1-yl]-2-fluoro-phenoxy]-4-methyl-thiazol-2-yl]-2,2,2-trifluoro-ethyl]-2-methyl-propane-2-sulfinamide (1.00 eq, 100 mg, 0.161 mmol) in 1,4-Dioxane (10 mL), was added HCl (4 M in dioxane) (4.0 mL). The mixture was stirred for 10 min at −20° C. under nitrogen. The reaction was concentrated to dryness. The crude was then purified by HPLC. The desired fractions were concentrated to dryness in vacuo to afford 2-(4-((2-(1-amino-2,2,2-trifluoroethyl)-4-methylthiazol-5-yl)oxy)-3-fluorophenyl)-4-(2,6-difluorobenzyl)-2,4-dihydro-3H-1,2,4-triazol-3-one (1 mg, 1.1% yield) as a white solid. MS (m/z): 516.2 [M+H]⁺.

| Compound | | | Appearance | | MS(m/z) |
|---|---|---|---|---|---|
| No. Method | Activity | Structure and Name | and Yield | ¹H NMR Data | [M + H]⁺ |
| Example 651 Method 123 | +++ | 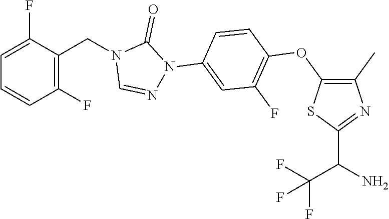 [SMILES: Cc1nc(C(N)C(F)(F)F)sc1Oc1ccc(-n2ncn(Cc3c(F)cccc3F)c2=O)cc1F] 2-(4-((2-(1-amino-2,2,2-trifluoroethyl)-4-methylthiazol-5-yl)oxy)-3- fluorophenyl)-4-(2,6-difluorobenzyl)-2,4-dihydro-3H-1,2,4-triazol-3-one | yellow solid | NA | 516.3 |

### Example 652: Method 124

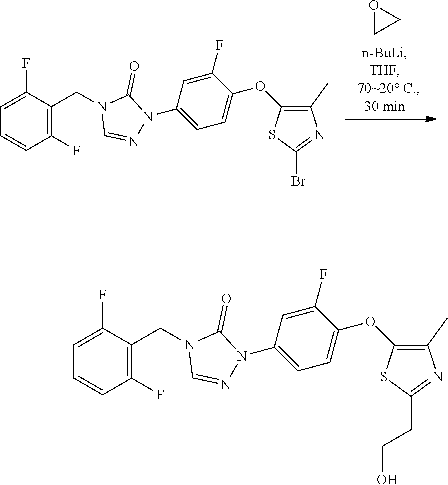

To a solution of 2-[4-(2-bromo-5-methyl-thiazol-4-yl)oxy-3-fluoro-phenyl]-4-[(2,6-difluorophenyl)methyl]-1,2,4-triazol-3-one (1.00 eq, 200 mg, 0.402 mmol) in THF (10 mL), was added n-BuLi (1.10 eq, 0.18 mL, 0.442 mmol) at −70° C. The mixture was stirred for 10 min at −70° C. under nitrogen. To this was added oxirane (10.0 eq, 1.0 mL, 4.02 mmol) at −70° C. The mixture was stirred for another 30 min at −70° C., and then slowly warmed to rt under nitrogen. The reaction was concentrated to dryness and the residue was taken up in EtOAc (200 mL) and the organics were washed with 2×100 mL water and then 1×100 mL saturated brine solution. The organics were then separated and dried (MgSO4) before concentration to dryness. The crude was then purified by flash column chromatography eluted with 50% EtOAc in PE. The desired fractions were concentrated to dryness in vacuo to afford 4-[(2,6-difluorophenyl)methyl]-2-[3-fluoro-4-[2-(2-hydroxyethyl)-5-methyl-thiazol-4-yl]oxy-phenyl]-1,2,4-triazol-3-one (6.0 mg, 2.71% yield) as a yellow oil MS (m/z): 463.2 [M+H]⁺. ¹H NMR (400 MHz, Chloroform-d) δ 7.89 (dd, J=12.4, 2.4 Hz, 1H), 7.79-7.73 (m, 1H), 7.60-7.56 (m, 1H), 7.43-7.31 (m, 1H), 7.08-6.94 (m, 3H), 4.97 (s, 2H), 3.74 (t, J=6.0 Hz, 2H), 2.82 (q, J=6.4 Hz, 2H), 2.21 (s, 3H).

| Compound | | | Appearance | | MS(m/z) |
|---|---|---|---|---|---|
| No. Method | Activity | Structure and Name | and Yield | ¹H NMR Data | [M + H]⁺ |
| Example 652 Method 124 | +++ | 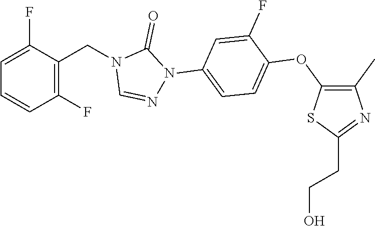 [SMILES: Cc1nc(CCO)sc1Oc1ccc(-n2ncn(Cc3c(F)cccc3F)c2=O)cc1F] 4-(2,6-difluorobenzyl)-2-(3-fluoro-4-((2-(2-hydroxyethyl)-4- methylthiazol-5-yl)oxy)phenyl)-2,4-dihydro-3H-1,2,4-triazol-3-one | white solid, yield: 2.7% | ¹H NMR (400 MHZ, Chloroform-d) δ 7.89 (dd, J = 12.4, 2.4 Hz, 1H), 7.79-7.73 (m, 1H), 7.60-7.56 (m, 1H), 7.43-7.31 (m, 1H), 7.08-6.94 (m, 3H), 4.97 (s, 2H), 3.74 (t, J = 6.0 Hz, 2H), 2.82 (q, J = 6.4 Hz, 2H), 2.21 (s, 3H). | 463.2 |

### Example 653: Method 125

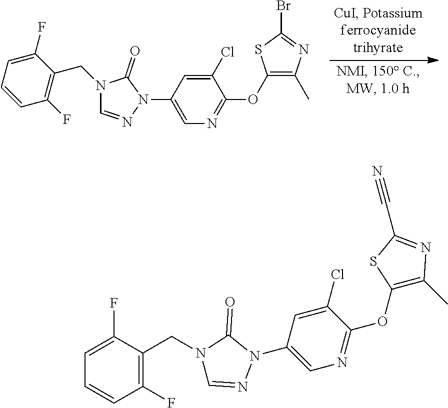 [SMILES: Cc1nc(Br)sc1Oc1ncc(-n2ncn(Cc3c(F)cccc3F)c2=O)cc1Cl ; Cc1nc(C#N)sc1Oc1ncc(-n2ncn(Cc3c(F)cccc3F)c2=O)cc1Cl]

To a solution of 2-[6-(2-bromo-4-methyl-thiazol-5-yl)oxy-5-chloro-3-pyridyl]-4-[(2,6-difluorophenyl)methyl]-1,2,4-triazol-3-one (1.00 eq, 100 mg, 0.194 mmol) in NMI (3 mL), was added potassium ferrocyanidetrihydrate (2.50 eq, 131 mg, 0.486 mmol), and CuI (2.17 eq, 80 mg, 0.421 mmol). The mixture was stirred for 1.0 h at 150° C. under microwave. The reaction was concentrated to dryness. The crude was then purified by flash column chromatography eluted with 50% ACN in H₂O. The desired fractions were concentrated to dryness in vacuo to afford 5-[[3-chloro-5-[4-[(2,6-difluorophenyl)methyl]-5-oxo-1,2,4-triazol-1-yl]-2-pyridyl]oxy]-4-methyl-thiazole-2-carbonitrile (6.0 mg, 0.0124 mmol, 6.37% yield) as a white solid. MS (m/z): 461.2 [M+H]⁺. ¹H NMR (400 MHz, Chloroform-d) δ 8.82 (d, J=2.4 Hz, 1H), 8.63 (d, J=2.4 Hz, 1H), 7.65 (s, 1H), 7.39 (t, J=7.6 Hz, 1H), 7.05-6.96 (m, 2H), 5.00 (s, 2H), 2.57 (s, 3H).

| Compound | | | Appearance | | MS(m/z) |
|---|---|---|---|---|---|
| No. Method | Activity | Structure and Name | and Yield | ¹H NMR Data | [M + H]⁺ |
| Example 653 Method 125 | +++ | 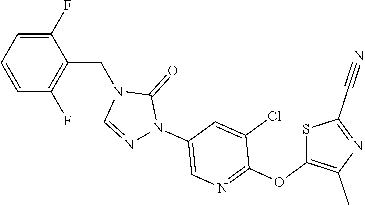 [SMILES: Cc1nc(C#N)sc1Oc1ncc(-n2ncn(Cc3c(F)cccc3F)c2=O)cc1Cl] 5-((3-chloro-5-(4-(2,6-difluorobenzyl)-5-oxo-4,5-dihydro-1H- 1,2,4-triazol-1-yl)pyridin-2-yl)oxy)-4-methylthiazole-2-carbonitrile | white solid, yield: 6.4% | ¹H NMR (400 MHZ, Chloroform-d) δ 8.82 (d, J = 2.4 Hz, 1H), 8.63 (d, J = 2.4 Hz, 1H), 7.65 (s, 1H), 7.39 (t, J = 7.6 Hz, 1H), 7.05-6.96 (m, 2H), 5.00 (s, 2H), 2.57 (s, 3H). | 461.2 |

### Example 654: Method 126

### Ethyl 4-((4-(4-(2,6-difluorobenzyl)-5-oxo-4,5-dihydro-1H-1,2,4-triazol-1-yl)-2-fluorophenoxy)methyl)thiazole-2-carboxylate

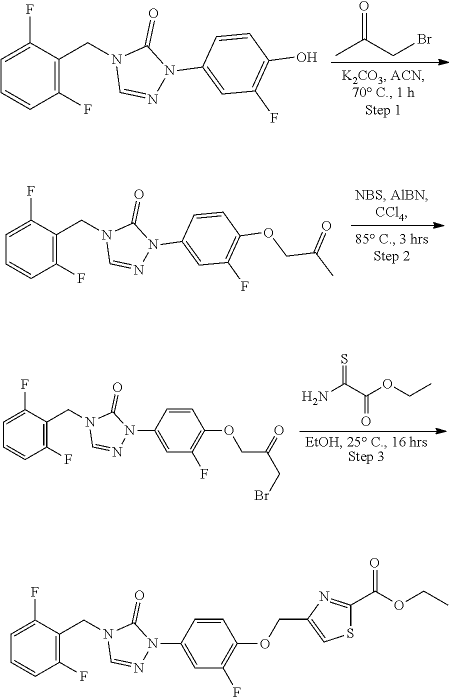

### Step 1: 4-(2,6-difluorobenzyl)-2-(3-fluoro-4-(2-oxopropoxy)phenyl)-2,4-dihydro-3H-1,2,4-triazol-3-one

To a solution of 4-[(2,6-difluorophenyl)methyl]-2-(3-fluoro-4-hydroxy-phenyl)-1,2,4-triazol-3-one (1.00 eq, 10 g, 31.1 mmol) in MeCN (200 mL), were added 1-bromopropan-2-one (1.20 eq, 5117 mg, 37.4 mmol), and K₂CO₃ (2.00 eq, 8591 mg, 62.3 mmol). The reaction mixture was stirred for 1 h at 70° C. under nitrogen. The solvent was evaporated to dryness. Water was added and it was extracted with EA (150 mL×3). The organic layers were evaporated to dryness to give 10 g white solid, yield: 85.14%. LC-MS (m/z): 378.4 [M+H]⁺. It was used for next step without further purification.

### Step 2: 2-(4-(3-bromo-2-oxopropoxy)-3-fluorophenyl)-4-(2,6-difluorobenzyl)-2,4-dihydro-3H-1,2,4-triazol-3-one

A solution of 4-(2,6-difluorobenzyl)-2-(3-fluoro-4-(2-oxopropoxy)phenyl)-2,4-dihydro-3H-1,2,4-triazol-3-one (500 mg, 1.33 mmol), NBS (259 mg, 1.46 mmol) and AIBN (21.8 mg, 0.13 mmol) in CCl₄ (10 mL) was stirred at 85° C. for 3 h under N₂. The mixture was evaporated in vacuo. The residue was purified by column (PE/EA=5:1) to afford compound 2-(4-(3-bromo-2-oxopropoxy)-3-fluorophenyl)-4-(2,6-difluorobenzyl)-2,4-dihydro-3H-1,2,4-tri azol-3-one (350 mg, crude) as a yellow oil. LC-MS (m/z): 457.3 [M+H]⁺.

### Step 3: ethyl4-((4-(4-(2,6-difluorobenzyl)-5-oxo-4,5-dihydro-1H-1,2,4-triazol-1-yl)-2-fluorophenoxy)methyl)thiazole-2-carboxylate

A solution of 2-(4-(3-bromo-2-oxopropoxy)-3-fluorophenyl)-4-(2,6-difluorobenzyl)-2,4-dihydro-3H-1,2,4-triazol-3-one (350 mg, 0.77 mmol), and ethyl 2-amino-2-thioxoacetate (306.5 mg, 2.30 mmol) in EtOH (10 mL) was stirred at 25° C. for 16 h under N₂. The mixture was evaporated in vacuo. The residue was purified by prep-HPLC with the following conditions: column: Sunfire 5 um 19-150 nm; Mobile phase B: ACN; Flow rate: 20 mL/min; Gradient: 20% B-70% B in 8 min; Detector: 220 nm. The fractions containing the desired product were collected at 55% B and concentrated under reduced pressure to afford ethyl4-((4-(4-(2,6-difluorobenzyl)-5-oxo-4,5-dihydro-1H-1,2,4-triazol-1-yl)-2-fluorophenoxy)methyl)thiazole-2-carboxylate (34 mg, 9.0% yield) as a white solid. LC-MS (m/z): 491.0 [M+H]⁺.

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 654 Method 126 | ++ | 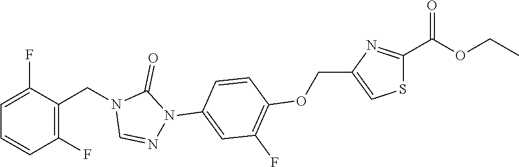 [SMILES: CCOC(=O)c1nc(COc2ccc(-n3ncn(Cc4c(F)cccc4F)c3=O)cc2F)cs1] ethyl4-((4-(4-(2,6-difluorobenzyl)-5-oxo-4,5-dihydro-1H-1,2,4-triazol-1-yl)- 2-fluorophenoxy)methyl)thiazole-2-carboxylate | white solid, yield: 9.0% | ¹H NMR (400 MHz, CDCl₃) δ 7.67-7.63 (m, 2H), 7.58 (s, 1H), 7.42-7.34 (m, 1H), 7.32 (s,1H), 7.27 (s, 1H), 6.99 (t, J = 7.6 Hz, 2H), 4.98 (s, 2H), 4.51-4.46 (m, 2H), 4.22 (s, 2H), 1.45 (t, J = 7.2 Hz, 3H). | 491.0 |

### Example 655: Method 127

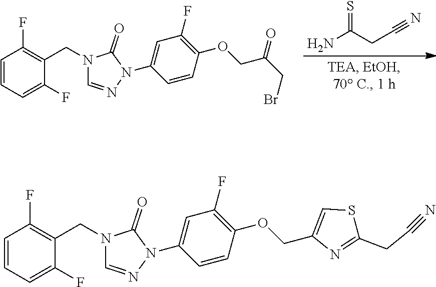 [SMILES: O=C(CBr)COc1ccc(-n2ncn(Cc3c(F)cccc3F)c2=O)cc1F ; N#CCC(N)=S ; N#CCc1nc(COc2ccc(-n3ncn(Cc4c(F)cccc4F)c3=O)cc2F)cs1]

To a solution of 2-[4-(3-bromo-2-oxo-propoxy)-3-fluoro-phenyl]-4-[(2,6-difluorophenyl) methyl]-1,2,4-triazol-3-one (1.00 eq, 150 mg, 0.329 mmol) in Ethanol (10 mL) were added 2-cyanothioacetamide (2.00 eq, 66 mg, 0.658 mmol), and TEA (3.00 eq, 0.14 mL, 0.986 mmol). The mixture was stirred for 1 h at 70° C. under nitrogen.

The reaction mixture was concentrated to dryness and the residue was taken up in EA (50 mL) and the organics were washed with 2×20 mL water and then 1×20 mL saturated brine solution. The organics were then separated and dried (MgSO₄) before concentration to dryness. The crude was then purified by flash column chromatography eluted with 50% ACN in H₂O. The desired fractions were concentrated to dryness in vacuo to afford 2-[4-[[4-[4-[(2,6-difluorophenyl)methyl]-5-oxo-1,2,4-triazol-1-yl]-2-fluoro-phenoxy]methyl]thiazol-2-yl]acetonitrile (6.0 mg, 3.95% yield) as a yellow solid. MS (m/z): 458.2 [M+H]⁺. ¹H NMR (400 MHz, Chloroform-d) δ 7.80 (dd, J=12.4, 2.4 Hz, 1H), 7.69 (dd, J=8.8, 2.0 Hz, 1H), 7.56 (s, 1H), 7.40 (s, 1H), 7.39-7.30 (m, 1H), 7.06 (t, J=8.8 Hz, 1H), 7.02-6.94 (m, 2H), 5.25 (d, J=1.2 Hz, 2H), 4.97 (s, 2H), 4.14 (s, 2H).

| Compound | | | Appear- | | |
|---|---|---|---|---|---|
| No. | | | ance | | MS(m/z) |
| Method | Activity | Structure and Name | and Yield | ¹H NMR Data | [M + H]⁺ |
| Example 655 Method 127 | +++ | 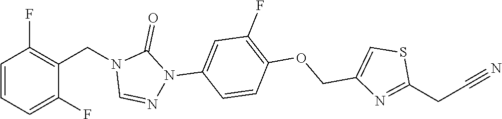 [SMILES: N#CCc1nc(COc2ccc(-n3ncn(Cc4c(F)cccc4F)c3=O)cc2F)cs1] 2-(4-((4-(4-(2,6-difluorobenzyl)-5-oxo-4,5-dihydro-1H-1,2,4-triazol-1-yl)- 2-fluorophenoxy)methyl)thiazol-2-yl)acetonitrile | yellow solid, yield: 21.6% | ¹H NMR (400 MHZ, Chloroform-d) δ 7.80 (dd, J = 12.4, 2.4 Hz, 1H), 7.69 (dd, J = 8.8, 2.0 Hz, 1H), 7.56 (s, 1H), 7.40 (s, 1H), 7.39-7.30 (m, 1H), 7.06 (t, J = 8.8 Hz, 1H), 7.02- 6.94 (m, 2H), | 458.2 |
| | | | | 5.25 (d, J = 1.2 | |
| | | | | Hz, 2H), 4.97 | |
| | | | | (s, 2H), 4.14 (s, 2H). | |

### Example 656: Method 128

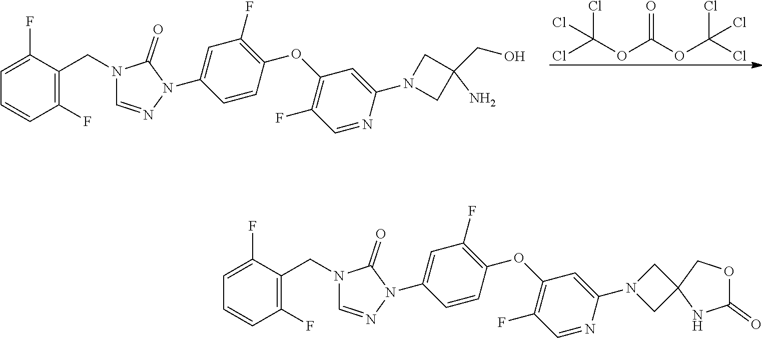 [SMILES: NC1(CO)CN(c2cc(Oc3ccc(-n4ncn(Cc5c(F)cccc5F)c4=O)cc3F)c(F)cn2)C1 ; O=C(OC(Cl)(Cl)Cl)OC(Cl)(Cl)Cl ; O=C1NC2(CO1)CN(c1cc(Oc3ccc(-n4ncn(Cc5c(F)cccc5F)c4=O)cc3F)c(F)cn1)C2]

To a solution of 2-[4-[[2-[3-amino-3-(hydroxymethyl)azetidin-1-yl]-5-fluoro-4-pyridyl]oxy]-3-fluoro-phenyl]-4-[(2,6-difluorophenyl)methyl]-1,2,4-triazol-3-one, and hydrochloride (1.00 eq, 64 mg, 0.116 mmol) in DCM (8 mL), was added TEA (2.00 eq, 0.032 mL, 0.232 mmol), and bis(trichloromethyl) carbonate (0.400 eq, 14 mg, 0.0465 mmol). The reaction mixture was stirred for 20 min at 0° C. under nitrogen. The reaction mixture was concentrated to dryness and the residue was taken up in EA (50 mL) and the organics were washed with 2×30 mL water and then 1×30 mL saturated brine solution. The organics were then separated and dried (MgSO₄) before concentration to dryness. The crude was then purified by flash column chromatography eluted with 50% ACN in H₂O. The desired fractions were concentrated to 8 dryness in vacuo to afford 2-[4-[4-[4-[(2,6-difluorophenyl)methyl]-5-oxo-1,2,4-triazol-1-yl]-2-fluoro-phenoxy]-5-fluoro-2-pyridyl]-7-oxa-2,5-diazaspiro[3.4]octan-6-one (14 mg, 21.33% yield) as a white solid. MS (m/z): 543.2 [M+H]⁺. ¹H NMR (400 MHz, Chloroform-d) δ 8.02 (d, J=2.8 Hz, 1H), 7.96 (dd, J=12.0, 2.4 Hz, 1H), 7.85 (ddd, J=9.2, 2.4, 1.6 Hz, 1H), 7.62 (d, J=0.8 Hz, 1H), 7.38 (tt, J=8.4, 6.4 Hz, 1H), 7.21 (t, J=8.8 Hz, 1H), 7.05-6.94 (m, 2H), 5.81 (brs, 1H), 5.59 (dd, J=5.6, 1.2 Hz, 1H), 4.99 (t, J=1.2 Hz, 2H), 4.55 (s, 2H), 4.06-3.97 (m, 4H).

| Com- | | | Appear- | | |
|---|---|---|---|---|---|
| pound | | | ance | | |
| No. | | | and | | MS(m/z) |
| Method | Activity | Structure and Name | Yield | ¹H NMR Data | [M + H]⁺ |
| Example 656 Method 128 | ++++ | 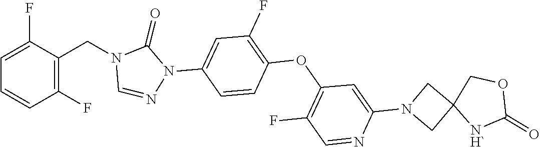 2-(4-(4-(4-(2,6-difluorobenzyl)-5-oxo-4,5-dihydro-1H-1,2,4-triazol- 1-yl)-2-fluorophenoxy)-5-fluoropyridin-2-yl)-7-oxa-2,5-diazaspiro[3.4]octan-6-one | white solid, yield: 21.3% | ¹H NMR (400 MHZ, Chloroform-d) δ 8.02 (d, J = 2.8 Hz, 1H), 7.96 (dd, J = 12.0, 2.4 Hz, 1H), 7.85 (ddd, J = 9.2, 2.4, 1.6 Hz, 1H), 7.62 (d, J = 0.8 Hz, 1H), 7.38 (tt, J = 8.4, 6.4 Hz, 1H), 7.21 (t, J = 8.8 Hz, 1H), 7.05- 6.94 (m, 2H), 5.81 | 543.2 |
| | | | | (brs, 1H), 5.59 | |
| | | | | (dd, J = 5.6, 1.2 Hz, | |
| | | | | 1H), 4.99 (t, | |
| | | | | J = 1.2 Hz, 2H), | |
| | | | | 4.55 (s, 2H), | |
| | | | | 4.06-3.97 (m, 4H). | |

### Example 657: Method 129

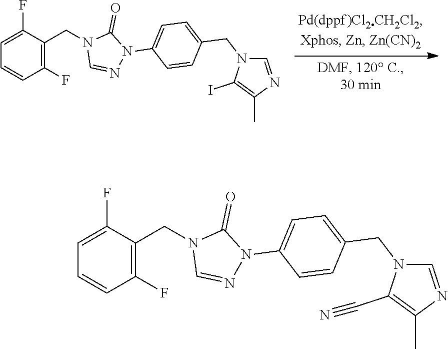 [SMILES: Cc1ncn(Cc2ccc(-n3ncn(Cc4c(F)cccc4F)c3=O)cc2)c1I ; Cc1ncn(Cc2ccc(-n3ncn(Cc4c(F)cccc4F)c3=O)cc2)c1C#N]

To a solution of 4-(2,6-difluorobenzyl)-2-(4-((5-iodo-4-methyl-1H-imidazol-1-yl)methyl)phenyl)-2,4-dihydro-3H-1,2,4-triazol-3-one (1.00 eq, 100 mg, 0.197 mmol) in DMF (10 mL), was added Zn (5.00 eq, 64 mg, 0.986 mmol), Pd(dppf)Cl₂, CH₂Cl₂ (0.300 eq, 48 mg, 0.0591 mmol), Xphos-g3 (0.600 eq, 100 mg, 0.118 mmol), and Zn(CN)₂ (4.00 eq, 140 mg, 0.789 mmol). The reaction mixture was stirred for 30 min at 120° C. under nitrogen. The reaction was concentrated to dryness and the residue was taken up in EA (100 mL) and the organics were washed with 2×50 mL water and then 1×50 mL saturated brine solution. The organics were then separated and dried (MgSO₄) before concentration to dryness. The crude was then purified by flash column chromatography eluted with 50% ACN in H₂O. The desired fractions were concentrated to dryness in vacuo to afford 3-[[4-[4-[(2,6-difluorophenyl)methyl]-5-oxo-1,2,4-triazol-1-yl]phenyl]methyl]-5-methyl-imidazole-4-carbonitrile (18 mg, 22.47% yield) as a white solid. MS (m/z): 407.2 [M+H]⁺. ¹H NMR (400 MHz, Chloroform-d) δ 8.07-7.98 (m, 2H), 7.64-7.56 (m, 2H), 7.41-7.33 (m, 1H), 7.34-7.29 (m, 2H), 7.03-6.92 (m, 2H), 5.16 (s, 2H), 4.97 (t, J=1.2 Hz, 2H), 2.39 (s, 3H).

| Compound | | | Appearance | | MS(m/z) |
|---|---|---|---|---|---|
| No. Method | Activity | Structure and Name | and Yield | ¹H NMR Data | [M + H]⁺ |
| Example 657 Method 129 | ++++ | 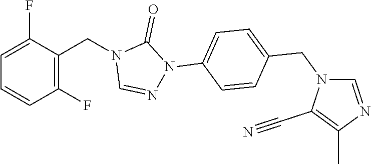 [SMILES: Cc1ncn(Cc2ccc(-n3ncn(Cc4c(F)cccc4F)c3=O)cc2)c1C#N] 1-(4-(4-(2,6-difluorobenzyl)-5-oxo-4,5-dihydro-1H-1,2,4- triazol-1-yl)benzyl)-4-methyl-1H-imidazole-5-carbonitrile | White solid, yield: 22.4% | ¹H NMR (400 MHZ, Chloroform-d) δ 8.07-7.98 (m, 2H), 7.64-7.56 (m, 2H), 7.41-7.33 (m, 1H), 7.34-7.29 (m, 2H), 7.03-6.92 (m, 2H), 5.16 (s, 2H), 4.97 (t, J = 1.2 Hz, 2H), 2.39 (s, 3H). | 407.2 |

### Example 658: Method 130

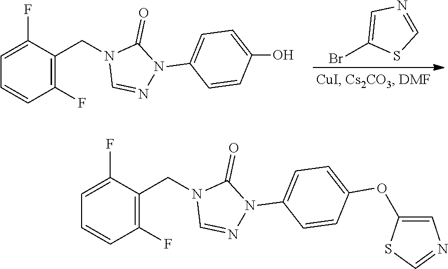 [SMILES: O=c1n(Cc2c(F)cccc2F)cnn1-c1ccc(O)cc1 ; Brc1cncs1 ; O=c1n(Cc2c(F)cccc2F)cnn1-c1ccc(Oc2cncs2)cc1]

To a solution of 4-[(2,6-difluorophenyl)methyl]-2-(4-hydroxyphenyl)-1,2,4-triazol-3-one (1.00 eq, 100 mg, 0.330 mmol) in DMF (4 mL), was added 5-bromothiazole (1.20 eq, 65 mg, 0.396 mmol), CuI (1.00 eq, 63 mg, 0.330 mmol), and Cs₂CO₃ (3.00 eq, 322 mg, 0.989 mmol). The reaction mixture was stirred for 0.5 h at 120° C. under nitrogen. The reaction was concentrated to dryness and the residue was taken up in EA (20 mL) and the organics were washed with 2×20 mL water and then 1×20 mL saturated brine solution. The organics were then separated and dried (MgSO₄) before concentration to dryness. The crude was then purified by flash column chromatography eluted with 80% ACN in H₂O. The desired fractions were concentrated to dryness in vacuo to afford 4-[(2,6-difluorophenyl)methyl]-2-(4-thiazol-5-yloxyphenyl)-1,2,4-triazol-3-one (27 mg, 20.13% yield) as a yellow solid. MS (m/z): 387.2 [M+H]⁺. ¹H NMR (400 MHz, DMSO-d₆) δ 8.75 (s, 1H), 8.30 (s, 1H), 7.91-7.83 (m, 2H), 7.63 (s, 1H), 7.48 (tt, J=8.4, 6.8 Hz, 1H), 7.29-7.21 (m, 2H), 7.21-7.10 (m, 2H), 4.97 (s, 2H).

| Compound | | | | | |
|---|---|---|---|---|---|
| No. | Activi- | | Appearance | | |
| Method | ty | Structure and Name | and Yield | ¹H NMR Data | MS (m/z) [M + H]⁺ |
| Example 658 Method 130 | ++++ | 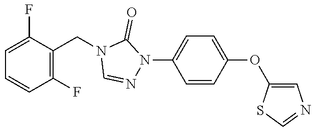 [SMILES: O=c1n(Cc2c(F)cccc2F)cnn1-c1ccc(Oc2cncs2)cc1] | yellow solid, yield: 20.13% | ¹H NMR (400 MHz, DMSO-d₆) δ 8.75 (s, 1H), 8.30 (s, 1H), 7.91- 7.83 (m, 2H), 7.63 (s, 1H), 7.48 (tt, J = 8.4, 6.8 Hz, 1H), 7.29- 7.21 (m, 2H), 7.21-7.10 (m, 2H), 4.97 (s, 2H). | 387.2 |
| | | 4-(2,6-difluorobenzyl)-2-(4-thi- | | | |
| | | azol-5-yloxy)phenyl)-2,4-dihydro- | | | |
| | | 3H-1,2,4-triazol-3-one | | | |

### Example 659: Method 131

### 2-(4-((2-aminothiazol-4-yl)methoxy)-3-fluorophenyl)-4-(2,6-difluorobenzyl)-2,4-dihydro-3H-1,2,4-triazol-3-one

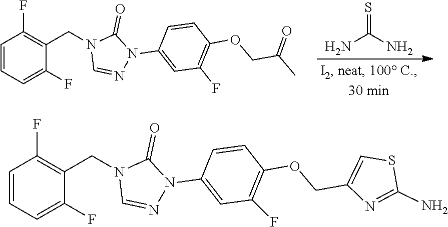

A mixture of 2-(4-acetonyloxy-3-fluoro-phenyl)-4-[(2,6-difluorophenyl)methyl]-1,2,4-triazol-3-one (1.1 g, 2.92 mmol), thiourea (333 mg, 4.37 mmol), and I₂ (1.00 eq, 738 mg, 2.92 mmol) was stirred for 30 min at 100° C. under nitrogen. Water was added to the mixture and the mixture was extracted with EtOAc (50 mL×3). The organic layers were evaporated to dryness and purified by prep-HPLC to give 126.5 mg white solid, yield: 10%. MS (m/z): 434.4 [M+H]⁺. ¹H NMR (400 MHz, CDCl₃) δ 8.05 (s, 1H), 7.78 (dd, J=12.4, 2.4 Hz, 1H), 7.70-7.63 (m, 1H), 7.55 (s, 1H), 7.40-7.32 (m, 1H), 7.08 (t, J=8.8 Hz, 1H), 7.01-6.95 (m, 2H), 6.54 (s, 1H), 5.01 (s, 2H), 4.96 (s, 2H).

| Compound | | | | | |
|---|---|---|---|---|---|
| No. | Activi- | | Appearance | | MS (m/z) |
| Method | ty | Structure and Name | and Yield | ¹H NMR Data | [M + H]⁺ |
| Example 659 Method 131 | +++ | 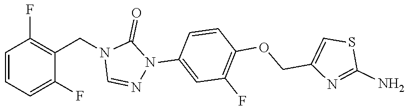 [SMILES: Nc1nc(COc2ccc(-n3ncn(Cc4c(F)cccc4F)c3=O)cc2F)cs1] | white solid, yield: 10% | ¹H NMR (400 MHz, CDCl₃) δ 8.05 (s, 1H), 7.78 (dd, J = 12.4, 2.4 Hz, 1H), 7.70-7.63 (m, 1H), 7.55 (s, 1H), 7.40-7.32 (m, 1H), 7.08 (t, J = 8.8 Hz, 1H), 7.01-6.95 (m, 2H), 6.54 (s, 1H), 5.01 (s, 2H), 4.96 (s, 2H). | 434.4 |
| | | 2-(4-((2-aminothiazol-4-yl)meth- | | | |
| | | oxy)-3-fluorophenyl)-4-(2,6-difluoro- | | | |
| | | benzyl)-2,4-dihydro-3H-1,2,4- | | | |
| | | triazol-3-one | | | |

### Example 660: Method 132

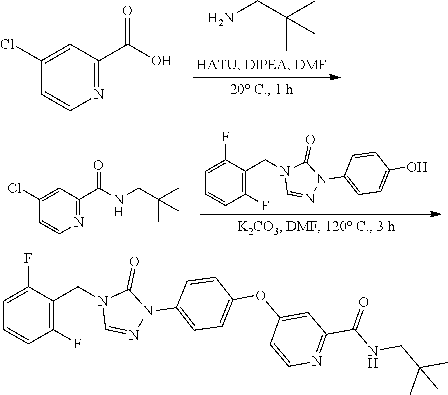

### Step 1: 4-chloro-N-neopentylpicolinamide

To a solution of 4-chloropicolinic acid (1.0 eq, 439 mg, 2.7 mmol) in DMF(20 mL), were added HATU (1.5 eq, 1600 mg, 4.2 mmol), DIPEA(2.0 eq, 724 mg, 5.6 mmol), and 2,2-dimethylpropan-1-amine(2.0 eq, 487 mg, 5.6 mmol). The mixture was stirred for 1 h at 20° C. under nitrogen. The reaction was diluted with EA(100 mL) and the organics were washed with 2×50 mL water and then 1×50 mL saturated brine solution. The organics were then separated and dried (MgSO₄) before concentration to dryness. The crude was then purified by flash column chromatography eluted with 70% ACN in H₂O to afford 4-chloro-N-neopentylpicolinamide (200 mg, 31.6% yield) as a yellow oil. MS (m/z): 227.2 [M+H]⁺

### Step 2: 4-(4-(4-(2,6-difluorobenzyl)-5-oxo-4,5-dihydro-1H-1,2,4-triazol-1-yl) phenoxy)-N-neopentylpicolinamide

To a solution of 4-chloro-N-neopentylpicolinamide (1.0 eq, 100 mg, 0.44 mmol) in DMF(5 mL), were added 4-(2,6-difluorobenzyl)-2-(4-hydroxyphenyl)-2,4-dihydro-3H-1,2,4-triazol-3-one (1.0 eq, 133 mg, 0.44 mmol), and K₂CO₃ (3.0 eq, 182 mg, 1.31 mmol). The mixture was stirred for 3 h at 120° C. under nitrogen. The reaction mixture was diluted with EA (100 mL) and the organics were washed with 2×50 mL water and then 1×50 mL saturated brine solution. The organics were then separated and dried (MgSO₄) before concentration to dryness. The crude was then purified by flash column chromatography eluted with 80% ACN in H₂O to afford 4-(4-(4-(2,6-difluorobenzyl)-5-oxo-4,5-dihydro-1H-1,2,4-triazol-1-yl)phenoxy)-N-neopentylpicolinamide(27 mg, 12.5% yield) as a light pink solid. MS (m/z): 494.2 [M+H]⁺. ¹H NMR (400 MHz, Chloroform-d) δ 8.38 (d, J=5.6 Hz, 1H), 8.21-8.13 (m, 1H), 8.08-8.00 (m, 2H), 7.74 (d, J=2.4 Hz, 1H), 7.60 (s, 1H), 7.37 (tt, J=8.4, 6.4 Hz, 1H), 7.18-7.09 (m, 2H), 7.03-6.96 (m, 2H), 6.94-6.90 (m, 1H), 4.99 (d, J=1.2 Hz, 2H), 3.25 (d, J=6.4 Hz, 2H), 0.98 (s, 9H).

| Compound | Ac- | | Appear- | | MS |
|---|---|---|---|---|---|
| No. | tiv- | | ance | | (m/z) |
| Method | ity | Structure and Name | and Yield | ¹H NMR Data | [M + H]⁺ |
| Example 660 Method 132 | +++ | 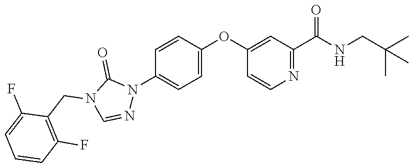 [SMILES: CC(C)(C)CNC(=O)c1cc(Oc2ccc(-n3ncn(Cc4c(F)cccc4F)c3=O)cc2)ccn1] | Light pink solid, yield: 12.5% | ¹H NMR (400 MHz, Chloroform-d) δ 8.38 (d, J = 5.6 Hz, 1H), 8.21-8.13 (m, 1H), 8.08-8.00 (m, 2H), 7.74 (d, J = 2.4 Hz, 1H), 7.60 (s, 1H), 7.37 (tt, J = 8.4, 6.4 Hz, 1H), 7.18- 7.09 (m, 2H), 7.03-6.96 (m, 2H), 6.94-6.90 (m, 1H), 4.99 (d, J = 1.2 Hz, 2H), 3.25 (d, J = 6.4 Hz, 2H), 0.98 (s, 9H). | 494.2 |
| | | 4-(4-(4-(2,6-difluorobenzyl)-5-oxo- | | | |
| | | 4,5-dihydro-1H-1,2,4-triazol- | | | |
| | | 1-yl)phenoxy)-N-neopentylpico- | | | |
| | | linamide | | | |

### Example 661: Method 133

### 4-(2,6-difluorobenzyl)-2-(4-((2-methoxypyridin-4-yl)oxy)phenyl)-2,4-dihydro-3H-1,2,4-tri azol-3-one

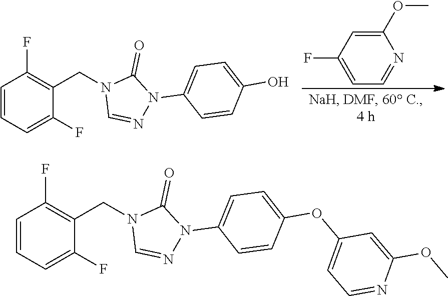

4-(2,6-difluorobenzyl)-2-(4-hydroxyphenyl)-2,4-dihydro-3H-1,2,4-triazol-3-one (100 mg, 0.329 mmoL) was dissolved in 3 mL DMF. NaH (19.7 mg, 0.493 mmoL) was added to the solution at 0° C. The mixture was stirred at room temperature for 30 min. 4-fluoro-2-methoxypyridine (50.2 mg, 0.395 mmoL) was then added to the mixture. The mixture was then stirred at 60° C. for 4h. Water was added to quench the reaction and the reaction mixture was extracted with EtOAc (15 mL×3). The organic layers were evaporated to dryness and purified by Prep-TLC (PE/EA=4/3) to give 38 mg light-yellow oil, yield: 28.2%. MS (m/z): 411.3 [M+H]⁺. ¹H NMR (400 MHz, CDCl₃) δ 8.15-7.99 (m, 3H), 7.61 (s, 1H), 7.52 (s, 1H), 7.15 (d, J=8.4 Hz, 2H), 7.03-6.95 (m, 2H), 6.65 (s, 1H), 6.26 (s, 1H), 4.99 (s, 2H), 4.04 (s, 3H).

The synthesis procedure of Example (Compounds) 662 was similar to the procedure of Example 661.

| Compound | | | | | |
|---|---|---|---|---|---|
| No. | Activi- | | Appearance | | MS (m/z) |
| Method | ty | Structure and Name | and Yield | ¹H NMR Data | [M + H]⁺ |
| Example 661 Method 133 | +++ | 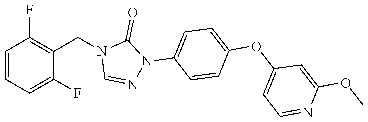 [SMILES: COc1cc(Oc2ccc(-n3ncn(Cc4c(F)cccc4F)c3=O)cc2)ccn1] | light-yellow oil, yield: 28.2% | ¹H NMR (400 MHz, CDCl₃) δ 8.15-7.99 (m, 3H), 7.61 (s, 1H), 7.52 (s, 1H), 7.15 (d, J = 8.4 Hz, 2H), 7.03-6.95 (m, 2H), 6.65 (s, 1H), 6.26 (s, 1H), 4.99 (s, 2H), 4.04 (s, 3H). | 411.3 |
| | | 4-(2,6-difluorobenzyl)-2-(4-((2- | | | |
| | | methoxypyridin-4-yl)oxy)phenyl)- | | | |
| | | 2,4-dihydro-3H-1,2,4-triazol- | | | |
| | | 3-one | | | |
| Example 662 Method 133 | +++ | 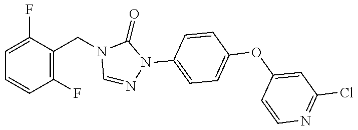 [SMILES: O=c1n(Cc2c(F)cccc2F)cnn1-c1ccc(Oc2ccnc(Cl)c2)cc1] | white solid, yield: 65.0% | ¹H NMR (400 MHz, Chloroform-d) δ 8.29-8.22 (m, 1H), 8.07 (d, J = 8.0 Hz, 2H), 7.61 (s, 1H), 7.40-7.35 (m, 1H), 7.15 (d, J = 8.4 Hz, 2H), 7.04-6.95 (m, 2H), 6.84 (s, 2H), 5.00 (s, 2H). | 415.2 |
| | | 2-(4-((2-chloropyridin-4-yl)oxy) | | | |
| | | phenyl)-4-(2,6-difluorobenzyl)- | | | |
| | | 2,4-dihydro-3H-1,2,4-triazol-3- | | | |
| | | one | | | |

### Example 663: Method 134

### 2-(4-((3-bromo-5-methyl-1H-1,2,4-triazol-1-yl)methyl)phenyl)-4-(2,6-difluorobenzyl)-2,4-dihydro-3H-1,2,4-triazol-3-one

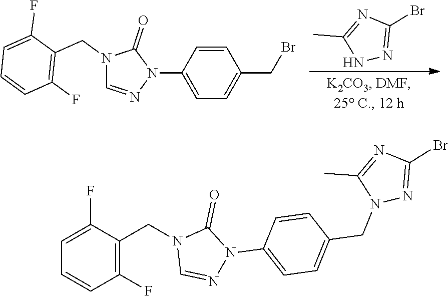

To a stirred solution of 2-(4-(bromomethyl)phenyl)-4-(2,6-difluorobenzyl)-2,4-dihydro-3H-1,2,4-triazol-3-one (500 mg, 3.08 mol) in DMF (10 mL) were added 3-bromo-5-methyl-1H-1,2,4-triazole (1.17 g, 3.08 mol) and potassium carbonate (1.27 g, 9.26 mmol) under N₂. The mixture was stirred at 25° C. for 12 hrs. The mixture was quenched by ice-water. The mixture was extracted with EA (5 mL×3). The combined organic layers were washed with water (10 mL×1), brine (20 mL), dried over Na₂SO₄ and concentrated. The residue was purified by column chromatography (PE/EA=50:1-5:1) to give compound 2-(4-((3-bromo-5-methyl-1H-1,2,4-triazol-1-yl)methyl)phenyl)-4-(2,6-difluorobenzyl)-2,4-dihy dro-3H-1,2,4-triazol-3-one (300 mg, 20.6% yield) as a yellow oil. MS (m/z): 461.0, 463.0 [M+H]⁺. ¹H NMR (400 MHz, DMSO-d₆): δ 8.31 (s, 1H), 7.86 (d, J 6.8 Hz, 1H), 7.46-7.50 (m, 2H), 7.34 (d, J 8.8 Hz, 1H), 7.16 (t, J 8.0 Hz, 1H), 5.35 (s, 2H), 4.97 (s, 2H), 2.43 (s, 3H)

| Compound | | | | | |
|---|---|---|---|---|---|
| No. | Activi- | | Appearance | | |
| Method | ty | Structure and Name | and Yield | ¹H NMR Data | MS (m/z) [M + H]⁺ |
| Example 663 Method 134 | +++ | 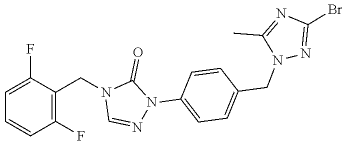 [SMILES: Cc1nc(Br)nn1Cc1ccc(-n2ncn(Cc3c(F)cccc3F)c2=O)cc1] | yellow oil, yield: 20.6% | ¹H NMR (400 MHz, DMSO-d₆): δ 8.31 (s, 1H), 7.86 (d, J = 6.8 Hz, 1H), 7.46-7.50 (m, 2H), 7.34 (d, J = 8.8 Hz, 1H), 7.16 (t, J = 8.0 Hz, 1H), 5.35 (s, 2H), 4.97 (s, 2H), 2.43 (s, 3H) | 461.0, 463.0 |
| | | 2-(4-((3-bromo-5-methyl-1H-1,2,4- | | | |
| | | triazol-1-yl)methyl)phenyl)- | | | |
| | | 4-(2,6-difluorobenzyl)-2,4-dihy- | | | |
| | | dro-3H-1,2,4-triazol-3-one | | | |

### Example 664: Method 135

### 1-(1-(4-(4-(2,6-difluorobenzyl)-5-oxo-4,5-dihydro-1H-1,2,4-triazol-1-yl)benzyl)-2-methyl-1H-imidazol-4-yl)-3-methylazetidine-3-carbonitrile

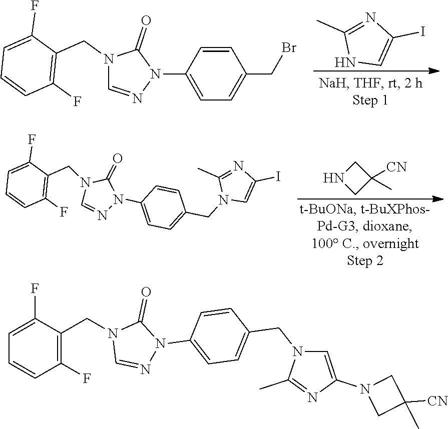

### Step 1: 4-(2,6-difluorobenzyl)-2-(4-((4-iodo-2-methyl-1H-imidazol-1-yl)methyl)phenyl)-2,4-dihydro-3H-1,2,4-triazol-3-one

To a stirred solution of 4-iodo-2-methyl-1H-imidazole (1.32 g, 6.3 mmol) in THF (200 mL) was added NaH (0.42 g, 10.6 mmol) at 0° C. The mixture was stirred at 0° C. for 30 min. 2-(4-(bromomethyl)phenyl)-4-(2,6-difluorobenzyl)-2,4-dihydro-3H-1,2,4-triazol-3-one (2 g, 5.3 mmol) was then added to the mixture at 0° C. The mixture was stirred at rt for 2 hr. The reaction was quenched with NH₄Cl solution (60 mL) and extracted with EA (10 mL×3). The combined organic layers were washed with brine (10 mL), dried over Na₂SO₄ and concentrated. The residue was purified by prep-TLC (PE/EA=1:1) to give 4-(2,6-difluorobenzyl)-2-(4-((4-iodo-2-methyl-1H-imidazol-1-yl)methyl)phenyl)-2,4-dihydro-3H-1,2,4-triazol-3-one (1 g, 37.7% yield) as a white solid. MS (m/z): 508.1 [M+H]⁺.

### Step 2: 1-(1-(4-(4-(2,6-difluorobenzyl)-5-oxo-4,5-dihydro-1H-1,2,4-triazol-1-yl)benzyl)-2-methyl-1H-imidazol-4-yl)-3-methylazetidine-3-carbonitrile

A solution of 4-(2,6-difluorobenzyl)-2-(4-((4-iodo-2-methyl-1H-imidazol-1-yl)methyl)phenyl)-2,4-dihydro-3H-1,2,4-triazol-3-one (100 mg, 0.20 mmol), 3-methylazetidine-3-carbonitrile (37.89 mg, 0.39 mmol), t-BuONa (56.82 mg, 0.59 mmol) and t-BuXPhos-Pd-G3 (78.29 mg, 0.1 mmol) in 1,4-dioxane (5 mL) was stirred at 100° C. overnight under N₂. The reaction mixture was quenched with H₂O (15 mL) and extracted with EA (5 mL×3). The combined organic layers were washed with brine (5 mL), dried over Na₂SO₄ and concentrated. The residue was purified by prep-TLC (DCMIMeOH=15:1) to afford the titled compound (14 mg, 14.90 yield) as yellow oil. MS (m/z): 476.1 [M+H]⁺. ¹H NMR (400 MHz, CD₃OD): δ7.99 (s, 1H), 7.88 (d, J=8.8 Hz, 2H), 7.44-7.42 (m, 1H), 7.23 (d, J=8.8 Hz, 2H), 7.05 (t, J=8.0 Hz, 2H), 6.30 (s, 1H), 5.05 (s, 2H), 5.03 (s, 2H), 4.00 (d, J=7.6 Hz, 2H), 3.67 (d, J=7.2 Hz, 2H), 2.26 (s, 3H), 1.67 (s, 3H).

The synthesis procedure of Examples (Compounds) 665-666 was similar to the procedure of Example 664.

| Compound | Ac- | | Appear- | | |
|---|---|---|---|---|---|
| No. | tivi- | | ance | | MS (m/z) |
| Method | ty | Structure and Name | and Yield | ¹H NMR Data | [M + H]⁺ |
| Example 664 Method 135 | ++ | 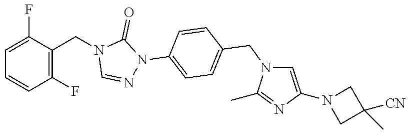 [SMILES: Cc1nc(N2CC(C)(C#N)C2)cn1Cc1ccc(-n2ncn(Cc3c(F)cccc3F)c2=O)cc1] | yellow oil, yield: 14.9% | ¹H NMR (400 MHz, CD₃OD): δ 7.99 (s, 1H), 7.88 (d, J = 8.8 Hz, 2H), 7.44-7.42 (m, 1H), 7.23 (d, J = 8.8 Hz, 2H), 7.05 (t, J = 8.0 Hz, 2H), 6.30 (s, 1H), 5.05 (s, 2H), 5.03 (s, 2H), 4.00 (d, J = 7.6 Hz, 2H), 3.67 (d, J = 7.2 Hz, 2H), 2.26 (s, 3H), 1.67 (s, 3H). | 476.1 |
| | | 1-(1-(4-(4-(2,6-difluorobenzyl)- | | | |
| | | 5-oxo-4,5-dihydro-1H-1,2,4-tri- | | | |
| | | azol-1-yl)benzyl)-2-methyl-1H-imid- | | | |
| | | azol-4-yl)-3-methylazetidine- | | | |
| | | 3-carbonitrile | | | |
| Example 665 Method 135 | ++ | 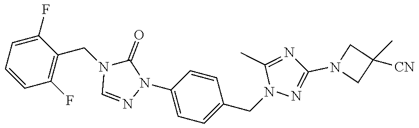 [SMILES: Cc1nc(N2CC(C)(C#N)C2)nn1Cc1ccc(-n2ncn(Cc3c(F)cccc3F)c2=O)cc1] | yellow oil, yield: 45% | ¹H NMR (400 MHz, CDCl₃): δ 7.98 (d, J = 6.0 Hz, 2H), 7.59 (s, 2H), 7.36-7.40 (m, 2H), 7.22 (d, J = 8.8 Hz, 2H), 7.00 (t, J = 8.0 Hz, 2H), 5.14 (s, 2H), 4.99 (s, 2H), 4.35 (d, J = 7.6 Hz, 2H), 3.91 (d, J = 7.6 Hz, 2H), 2.32 (s, 3H), 1.77 (s, 3H). | 447.1 |
| | | 1-(1-(4-(4-(2,6-difluorobenzyl)- | | | |
| | | 5-oxo-4,5-dihydro-1H-1,2,4-tri- | | | |
| | | azol-1-yl)benzyl)-5-methyl-1H-1,2,4- | | | |
| | | triazol-3-yl)-3-methylazetidine- | | | |
| | | 3-carbonitrile | | | |
| Example 666 Method 135 | ++++ | 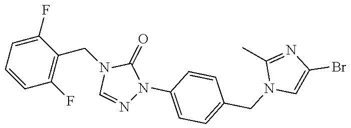 [SMILES: Cc1nc(Br)cn1Cc1ccc(-n2ncn(Cc3c(F)cccc3F)c2=O)cc1] | yellow solid, yield: 48.2% | ¹H NMR (400 MHz, DMSO-d₆): δ 8.30 (s, 1H), 7.86 (d, J = 8.8 Hz, 2H), 7.49-7.44 (m, 1H), 7.30-7.28 (m, 3H), 7.16 (t, J = 8.0 Hz, 2H), 5.13 (s, 2H), 4.98 (s, 2H), 2.23 (s, 3H). | 460.0, 462.0 |
| | | 2-(4-((4-bromo-2-methyl-1H-imid- | | | |
| | | azol-1-yl)methyl)phenyl)-4-(2,6- | | | |
| | | difluorobenzyl)-2,4-dihydro- | | | |
| | | 3H-1,2,4-triazol-3-one | | | |

### Example 667: Method 136

### 5-(2-fluoro-4-(5-(4-fluorophenyl)-3-oxo-6,7-dihydro-3H-pyrrolo[2,1-c][1,2,4]triazol-2(5H)-yl)phenoxy)-4-methylthiazole-2-carboxamide

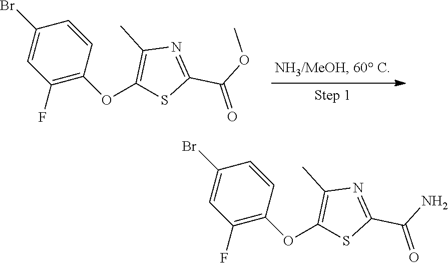 [SMILES: COC(=O)c1nc(C)c(Oc2ccc(Br)cc2F)s1 ; Cc1nc(C(N)=O)sc1Oc1ccc(Br)cc1F]

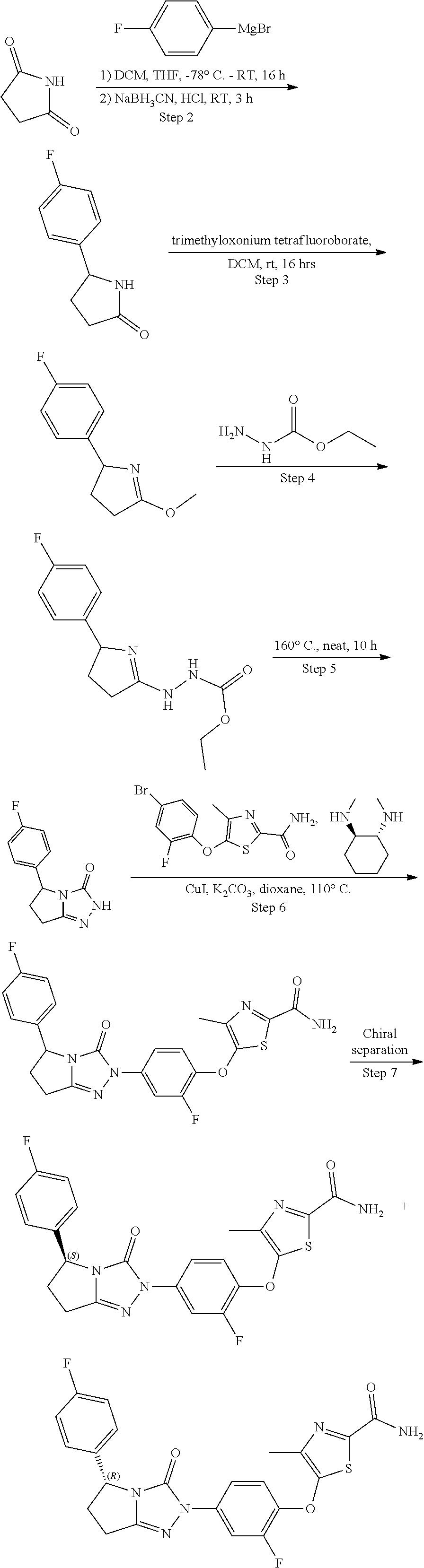

### Step 1: 5-(4-bromo-2-fluorophenoxy)-4-methylthiazole-2-carboxamide

To a solution of methyl 5-(4-bromo-2-fluoro-phenoxy)-4-methyl-thiazole-2-carboxylate (1.00 eq, 100 mg, 0.289 mmol) in 1 mL of (NH₃/MeOH, 7M/L) at room temperature under N₂. After addition, the mixture was stirred at 70° C. for 1 h. Then the mixture was concentrated to give the product 5-(4-bromo-2-fluoro-phenoxy)-4-methyl-thiazole-2-carboxamide (90 mg, 84.67% yield) as a yellow solid. MS (m/z): 331.2 [M+H]⁺.

### Step 2: 5-(4-fluorophenyl)pyrrolidin-2-one

To a stirred solution of pyrrolidine-2,5-dione (6 g, 60.6 mmol) in DCM (40 mL) was added the solution of (4-fluorophenyl)magnesium bromide in THF (1 N, 120 mL, 120 mmol) at −78° C. under Ar. The resulted mixture was allowed to naturally warmed to rt and stirred overnight. At which time, NaBH₃CN (4.57 g, 72.7 mmol) was added in and the reaction mixture was acidified to PH=4 with 6N HCl. The reaction mixture was stirred for additional 2 h at r.t. The resulted mixture was diluted with water (200 mL). The resulted mixture was extracted with EtOAc (3×80 mL). The combined organic layers were washed with brine (100 mL), dried over anhydrous Na₂SO₄, and after filtration, the filtrate was concentrated under reduced pressure. The residue was purified by silica gel column chromatography, eluted with (PE:EtOAc) (1:1) to afford 5-(4-fluorophenyl)pyrrolidin-2-one (3 g, 27.6% yield) as a pale yellow solid. MS (m/z): 180.1 [M+H]⁺.

### Step 3: 2-(4-fluorophenyl)-5-methoxy-3,4-dihydro-2H-pyrrole

To a stirred solution of 5-(4-fluorophenyl)pyrrolidin-2-one (1.5 g, 8.3 mmol) in DCM (20 mL) were added trimethyloxonium tetrafluoroborate (1.49 g, 10.1 mmol) at rt. The resulted mixture was stirred at rt for 16 hours. The mixture was used in next step directly without further purification. MS (m/z): 194.1 [M+H]⁺.

### Step 4: ethyl 2-(2-(4-fluorophenyl)-3,4-dihydro-2H-pyrrol-5-yl)hydrazine-1-carboxylate

To a stirred solution of last step was added ethyl hydrazinecarboxylate (1.06 g, 10.1 mmol) at r.t. The resulted mixture was stirred for additional 2 h at rt. The mixture was washed with sat. NaHCO₃ solution (20 mL). The aqueous layers were extracted with EtOAc (3×15 mL). The combined organic layers were washed with brine (30 mL), dried over anhydrous Na₂SO₄, and after filtration, the filtrate was concentrated under reduced pressure. The residue was purified by silica gel column chromatography, eluted with (PE:EtOAc) (2:1) to afford ethyl 2-(2-(4-fluorophenyl)-3,4-dihydro-2H-pyrrol-5-yl)hydrazine-1-carboxylate (0.9 g, 43.6% yield) as a white solid. LC-MS (m/z): 266.1[M+H]⁺.

### Step 5: 5-(4-fluorophenyl)-2,5,6,7-tetrahydro-3H-pyrrolo[2,1-c][1,2,4]triazol-3-one

Ethyl 2-(2-(4-fluorophenyl)-3,4-dihydro-2H-pyrrol-5-yl)hydrazine-1-carboxylate (900 mg, 3.39 mmol) was stirred for 10 h at 160° C. without a solvent. It was purified by silica gel column chromatography, eluted with (DCM:MeOH=20:1) to afford the titled compound (120 mg, 16.14% yield) as a yellow solid. LC-MS (m/z): 220.1 [M+H]⁺. ¹H NMR (400 MHz, DMSO) δ 11.26 (s, 1H), 7.38-7.11 (m, 4H), 5.17 (dd, J=8.1, 4.5 Hz, 1H), 3.03-2.88 (m, 1H), 2.88-2.78 (m, 1H), 2.78-2.66 (m, 1H), 2.34-2.18 (m, 1H).

### Step 6: 5-(2-fluoro-4-(5-(4-fluorophenyl)-3-oxo-6,7-dihydro-3H-pyrrolo[2,1-c][1,2,4]triazol-2(5H)-yl)phenoxy)-4-methylthiazole-2-carboxamide

To a solution of 5-(4-fluorophenyl)-2,5,6,7-tetrahydropyrrolo[2,1-c][1,2,4]triazol-3-one (1.00 eq, 74 mg, 0.339 mmol), 5-(4-bromophenoxy)-4-methyl-thiazole-2-carboxamide (1.00 eq, 106 mg, 0.339 mmol), (1R,2R)—N,N′-Dimethyl-1,2-cyclohexanediamine (1.00 eq, 48 mg, 0.339 mmol), and K₂CO₃ (2.00 eq, 94 mg, 0.678 mmol) in DMF (3 mL), was added CuI (0.500 eq, 32 mg, 0.169 mmol) at room temperature under N₂. After addition, the mixture was stirred at 110° C. for 1 h. Then the reaction mixture was added with water (1 mL) and extracted with EtOAc (1 mL×3). The organics were then combined and dried (Na₂SO₄) before concentration to dryness. The crude was purified by prep-HPLC to give the product 5-[4-[5-(4-fluorophenyl)-3-oxo-6,7-dihydro-5H-pyrrolo[2,1-c][1,2,4]triazol-2-yl]phenoxy]-4-m ethyl-thiazole-2-carboxamide (35 mg, 21.74% yield) as a white solid. MS (m/z): 470.2 [M+H]⁺.

Step 7: The 5-(2-fluoro-4-(5-(4-fluorophenyl)-3-oxo-6,7-dihydro-3H-pyrrolo[2,1-c][1,2,4]triazol-2(5H)-yl)phenoxy)-4-methylthiazole-2-carboxamide (35 mg) was purified by chiral separation by SFC under basic condition to give two products.

### (S)-5-(2-fluoro-4-(5-(4-fluorophenyl)-3-oxo-6,7-dihydro-3H-pyrrolo[2,1-c][1,2,4]triazol-2(5H)-yl)phenoxy)-4-methylthiazole-2-carboxamide

9.7 mg, 27.71% yield, white solid. MS (m/z): 470.2 [M+H]⁺. ¹H NMR (400 MHz, DMSO-d6) δ 8.10 (s, 1H), 7.87 (dd, J=12.8, 2.4 Hz, 1H), 7.80 (s, 1H), 7.73-7.65 (m, 1H), 7.48-7.31 (m, 3H), 7.27-7.17 (m, 2H), 5.33 (dd, J=7.2, 5.2 Hz, 1H), 3.10-2.82 (m, 3H), 2.43-2.34 (m, 1H), 2.31 (s, 3H).

### (R)-5-(2-fluoro-4-(5-(4-fluorophenyl)-3-oxo-6,7-dihydro-3H-pyrrolo[2,1-c][1,2,4]triazol-2(5H)-yl)phenoxy)-4-methylthiazole-2-carboxamide

9.7 mg, 27.71% yield, white solid. MS (m/z): 470.2 [M+H]⁺. ¹H NMR (400 MHz, DMSO-d6) δ 8.10 (s, 1H), 7.87 (dd, J=12.8, 2.6 Hz, 1H), 7.80 (s, 1H), 7.73-7.65 (m, 1H), 7.45-7.33 (m, 3H), 7.26-7.17 (m, 2H), 5.33 (dd, J=7.4, 5.2 Hz, 1H), 3.11-2.81 (m, 3H), 2.40-2.32 (m, 1H), 2.32 (s, 3H).

The synthesis procedure of Examples (Compounds) 668-669 was similar to the procedure of Example 667.

| Compoud No. | Activi- | | Appearance | | MS (m/z) |
|---|---|---|---|---|---|
| Method | ty | Structure and Name | and Yield | ¹H NMR Data | [M + H]⁺ |
| Example 667 Method 136 | ++++ | 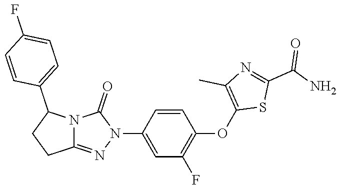 [SMILES: Cc1nc(C(N)=O)sc1Oc1ccc(-n2nc3n(c2=O)C(c2ccc(F)cc2)CC3)cc1F] | White solid, yield: 27.71% | NA | 470.2 |
| | | 5-(2-fluoro-4-(5-(4-fluorophenyl)- | | | |
| | | 3-oxo-6,7-dihydro-3H-pyrrolo[1,2- | | | |
| | | c][1,2,4]triazol-2(5H)-yl)phenox- | | | |
| | | y)-4-methylthiazole-2-carboxamide | | | |
| Example 668 Method 136 single unknown stereoisomer | ++++ | 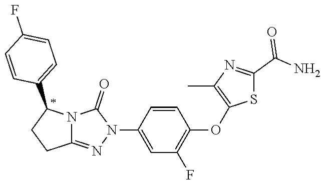 [SMILES: Cc1nc(C(N)=O)sc1Oc1ccc(-n2nc3n(c2=O)[C@H](c2ccc(F)cc2)CC3)cc1F] | | ¹H NMR (400 MHz, DMSO-d₆) δ 8.10 (s, 1H), 7.87 (dd, J = 12.8, 2.4 Hz, 1H), 7.80 (s, 1H), 7.73-7.65 (m, 1H), 7.48-7.31 (m, 3H), 7.27- 7.17 (m, 2H), 5.33 (dd, J = 7.2, 5.2 Hz, 1H), 3.10-2.82 (m, 3H), 2.43- 2.34 (m, 1H), 2.31 (s, 3H). | 470.2 |
| | | (S)-5-(2-fluoro-4-(5-(4-fluorophe- | | | |
| | | nyl)-3-oxo-6,7-dihydro-3H-pyrrolo | | | |
| | | [2,1-c][1,2,4]triazol-2(5H)-yl)phe- | | | |
| | | noxy)-4-methylthiazole-2-carbox- | | | |
| | | amide | | | |
| Example 669 Method 136 single unknown stereoisomer | ++ | 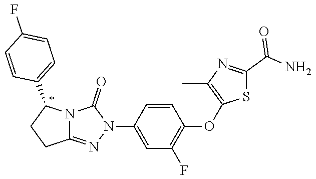 [SMILES: Cc1nc(C(N)=O)sc1Oc1ccc(-n2nc3n(c2=O)[C@@H](c2ccc(F)cc2)CC3)cc1F] | | ¹H NMR (400 MHz, DMSO-d₆) δ 8.10 (s, 1H), 7.87 (dd, J = 12.8, 2.6 Hz, 1H), 7.80 (s, 1H), 7.73-7.65 (m, 1H), 7.45-7.33 (m, 3H), 7.26- 71.7 (m, 2H), 5.33 (dd, J = 7.4, 5.2 Hz, 1H), 3.11-2.81 (m, 3H), 2.40- 2.32 (m, 1H), 2.32 (s, 3H). | 470.2 |
| | | (R)-5-(2-fluoro-4-(5-(4-fluorophe- | | | |
| | | nyl)-3-oxo-6,7-dihydro-3H-pyrrolo | | | |
| | | [2,1-c][1,2,4]triazol-2(5H)-yl)phe- | | | |
| | | noxy)-4-methylthiazole-2-carbox- | | | |
| | | amide | | | |

### Example 670: Method 137

### 5-(2-fluoro-4-(5-(4-fluorophenyl)-3-oxo-6,7-dihydro-3H-pyrrolo[2,1-c][1,2,4]triazol-2(5H)-yl)phenoxy)-4-methylthiazole-2-carboxamide

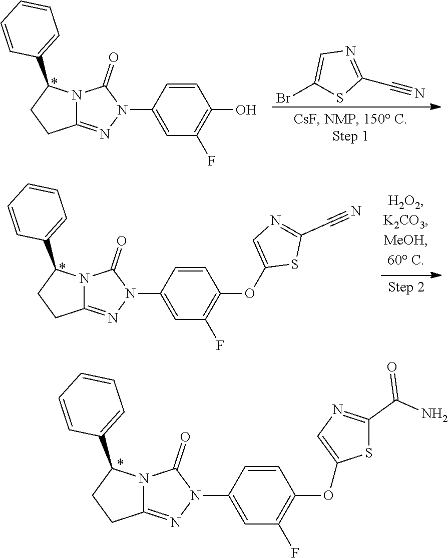 [SMILES: O=c1n(-c2ccc(O)c(F)c2)nc2n1[C@H](c1ccccc1)CC2 ; N#Cc1ncc(Br)s1 ; N#Cc1ncc(Oc2ccc(-n3nc4n(c3=O)[C@H](c3ccccc3)CC4)cc2F)s1 ; NC(=O)c1ncc(Oc2ccc(-n3nc4n(c3=O)[C@H](c3ccccc3)CC4)cc2F)s1]

### Step 1: 5-(4-bromo-2-fluorophenoxy)-4-methylthiazole-2-carboxamide

To a solution of 5-bromothiazole-2-carbonitrile (1.00 eq, 100 mg, 0.528 mmol), 2-(3-fluoro-4-hydroxy-phenyl)-5-phenyl-6,7-dihydro-5H-pyrrolo[2,1-c][1,2,4]triazol-3-one (1.00 eq, 164 mg, 0.528 mmol) in NMP (5 mL), and CsF (2.00 eq, 161 mg, 1.06 mmol) was added at room temperature under N₂. After addition, the mixture was stirred at 150° C. for 1 h. Then the mixture was added with water (2 mL) and extracted with EtOAc (1 mL×3). The organics were then combined and dried (Na₂SO₄) before concentration to dryness. The crude was purified with prep-TLC (PE/EtOAc=1:1) to give the product 5-[2-fluoro-4-(3-oxo-5-phenyl-6,7-dihydro-5H-pyrrolo[2,1-c][1,2,4]triazol-2-yl)phenoxy]thiaz ole-2-carbonitrile (45 mg, 18.07% yield) as a white solid. MS (m/z): 420.2 [M+H]⁺.

### Step 2: 5-(2-fluoro-4-(5-(4-fluorophenyl)-3-oxo-6,7-dihydro-3H-pyrrolo[2,1-c][1,2,4]triazol-2(5H)-yl)phenoxy)-4-methylthiazole-2-carboxamide

To a solution of 5-[2-fluoro-4-[(5S)-3-oxo-5-phenyl-6,7-dihydro-5H-pyrrolo[2,1-c][1,2,4]triazol-2-yl]phenoxy]-4-methyl-thiazole-2-carbonitrile (1.00 eq, 130 mg, 0.300 mmol) in MeOH (5 mL), and H₂O₂(5 mL), K₂CO₃ (3.00 eq, 83 mg, 0.600 mmol) was added at room temperature under N₂. After addition, the mixture was stirred at 50° C. for 1 h. Then the mixture was added with water (5 mL) and extracted with EtOAc (5 mL×3). The organics were then combined and dried (Na₂SO₄) before concentration to dryness. The crude was purified by prep-HPLC to give the product 5-[2-fluoro-4-[(5S)-3-oxo-5-phenyl-6,7-dihydro-5H-pyrrolo[2,1-c][1,2,4]triazol-2-yl]phenoxy]-4-methyl-thiazole-2-carboxamide (65 mg, 0.137 mmol, 46.0% yield) as a white solid. MS (m/z): 438.2 [M+H]⁺. ¹H NMR (400 MHz, DMSO-d6) δ 8.14 (s, 1H), 7.90 (dd, J=12.8, 2.5 Hz, 1H), 7.83 (s, 1H), 7.78-7.71 (m, 1H), 7.65 (s, 1H), 7.54 (t, J=9.2 Hz, 1H), 7.44-7.30 (m, 5H), 5.32 (dd, J=7.8, 4.8 Hz, 1H), 3.10-2.82 (m, 3H), 2.42-2.28 (m, 1H).

| Compound | | | Appear- | | |
|---|---|---|---|---|---|
| No. | Activi- | | ance | | |
| Method | ty | Structure and Name | and Yield | ¹H NMR Data | MS (m/z) [M + H]⁺ |
| Example 670 Method 137 single unknown stereoisomer | ++++ | 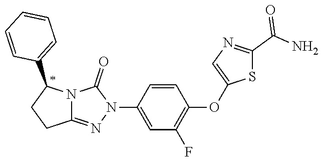 [SMILES: NC(=O)c1ncc(Oc2ccc(-n3nc4n(c3=O)[C@H](c3ccccc3)CC4)cc2F)s1] | White solid, yield: 46.0% | ¹H NMR (400 MHz, DMSO-d₆) δ 8.14 (s, 1H), 7.90 (dd, J = 12.8, 2.5 Hz, 1H), 7.83 (s, 1H), 7.78-7.71 (m, 1H), 7.65 (s, 1H), 7.54 (t, J = 9.2 Hz, 1H), 7.44-7.30 (m, 5H), 5.32 (dd, J = 7.8, 4.8 Hz, 1H), 3.10-2.82 (m, 3H), 2.42-2.28 (m, 1H). | 438.2 |
| | | (S)-5-(2-fluoro-4-(3-oxo-5-phenyl- | | | |
| | | 6,7-dihydro-3H-pyrrolo[2,1-c][1,2,4] | | | |
| | | triazol-2(5H)-yl)phenoxy)thia- | | | |
| | | zole-2-carboxamide | | | |

### Example 671: Method 138

### 4-(2,6-difluorobenzyl)-2-(4-((5-methoxypyridin-3-yl)oxy)phenyl)-2,4-dihydro-3H-1,2,4-tri azol-3-one

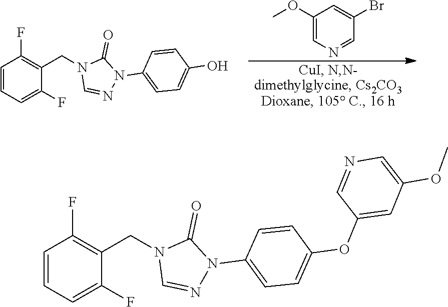

To a stirred solution 4-[(2,6-difluorophenyl)methyl]-2-(4-hydroxyphenyl)-1,2,4-triazol (100 mg, 0.32 mmol) and 3-bromo-5-methoxypyridine (93.0 mg, 0.49 mmol) in dioxane (15 mL) were added Cs₂CO₃ (322.3 mg, 0.98 mmol), copper(I) iodide (31.4 mg, 0.16 mmol), and N,N-dimethylglycine (16.5 mg, 0.16 mmol). The reaction mixture was stirred at 105° C. for 16 h under N₂. The resulted mixture was diluted with water (20 mL). The resulted mixture was extracted with EtOAc (3×10 mL). The combined organic layers were washed with brine (10 mL), dried over anhydrous Na₂SO₄, and after filtration, the filtrate was concentrated under reduced pressure. The residue was purified by silica gel column chromatography, and eluted with (MeOH:DCM=1-10%) to afford 4-(2,6-difluorobenzyl)-2-(4-((5-methoxypyridin-3-yl)oxy)phenyl)-2,4-dihydro-3H-1,2,4-triazol-3-one (20 mg, 14.8% yield) as a white solid. MS (m/z): 410.6 [M+H]⁺. ¹H NMR (400 MHz, CDCl₃): δ 8.09 (s, 1H), 7.99-7.91 (m, 2H), 7.57 (s, 1H), 7.35 (m, J=13.1, 7.5, 4.2 Hz, 1H), 7.25 (s, 1H), 7.11-7.03 (m, 2H), 7.02-6.92 (m, 2H), 6.82 (s, 2H), 4.97 (s, 2H), 3.81 (s, 3H).

| Compound | | | | | |
|---|---|---|---|---|---|
| No. | Activi- | | Appearance | | |
| Method | ty | Structure and Name | and Yield | ¹H NMR Data | MS (m/z) [M + H]⁺ |
| Example 671 Method 138 | ++++ | 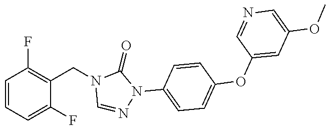 [SMILES: COc1cncc(Oc2ccc(-n3ncn(Cc4c(F)cccc4F)c3=O)cc2)c1] | white solid, yield: 14.8% | ¹H NMR (400 MHz, DMSO-d₆) δ 8.09 (s, 1H), 7.99-7.91 (m, 2H), 7.57 (s, 1H), 7.35 (m, J = 13.1, 7.5, 4.2 Hz, 1H), 7.25 (s, 1H), 7.11-7.03 (m, 2H), 7.02- 6.92 (m, 2H), 6.82 (s, 2H), 4.97 (s, 2H), 3.81 (s, 3H). | 410.6 |
| | | 4-(2,6-difluorobenzyl)-2-(4-((5- | | | |
| | | methoxypyridin-3-yl)oxy)phenyl)- | | | |
| | | 2,4-dihydro-3H-1,2,4-triazol- | | | |
| | | 3-one | | | |

### Example 672: Method 139

### 4-(2,6-difluorobenzyl)-2-(4-((3-fluoro-2-(7-oxa-2-azaspiro[3.5]nonan-2-yl)pyridin-4-yl)oxy)phenyl)-2,4-dihydro-3H-1,2,4-triazol-3-one

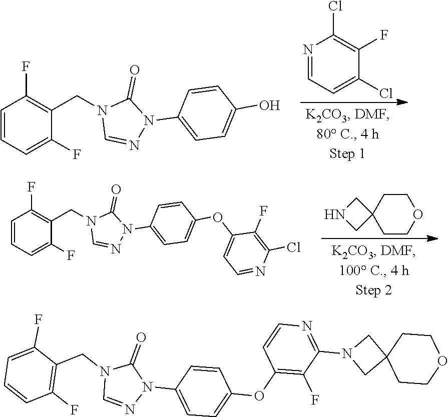

### Step 1: 2-(4-((2-chloro-3-fluoropyridin-4-yl)oxy)phenyl)-4-(2,6-difluorobenzyl)-2,4-dihydro-3H-1,2,4-triazol-3-one

To a stirred solution of 4-[(2,6-difluorophenyl)methyl]-2-(4-hydroxyphenyl)-1,2,4-triazol-3-one (500 mg, 1.64 mmol) and 2,4-dichloro-3-fluoropyridine(410.48 mg, 2.47 mmol) in DMF (20 mL) was added potassium carbonate (455.73 mg, 3.29 mmol) under N₂. The reaction mixture was stirred at 80° C. for 4h, washed with water (100 mL), extracted with EA(2×20 mL) and the combined organic layers were washed with brine, dried (Na₂SO₄), filtered and concentrated under reduced pressure. The residue was purified by a silica gel chromatography with EA/PE (1-15%) to obtain 2-(4-((2-chloro-3-fluoropyridin-4-yl)oxy)phenyl)-4-(2,6-difluorobenzyl)-2,4-dihydro-3H-1,2,4-triazol-3-one (225 mg, 30.7% yield) as a white solid. MS (m/z): 432.0 [M+H]⁺.

### Step 2: 4-(2,6-difluorobenzyl)-2-(4-((3-fluoro-2-(7-oxa-2-azaspiro[3.5]nonan-2-yl) pyridin-4-yl)oxy)phenyl)-2,4-dihydro-3H-1,2,4-triazol-3-one

2-{4-[(2-chloro-3-fluoropyridin-4-yl)oxy]phenyl}-4-[(2,6-difluorophenyl)methyl]-1,2,4-triazol-3-one (100 mg, 0.23 mmol) in DMF (15 mL) was added K₂CO₃ (95.82 mg, 0.69 mmol) and 7-oxa-2-azaspiro[3.5]nonane (35.27 mg, 0.27 mmol). The reaction mixture was stirred at 100° C. for 4 h, washed with water (20 mL), extracted with EA(2×20 mL) and the combined organic layers were washed with brine, dried (Na₂SO₄), filtered and concentrated under reduced pressure. The residue was purified by silica gel chromatography with EA/PE (1-15%) to get 4-(2,6-difluorobenzyl)-2-(4-((3-fluoro-2-(7-oxa-2-azaspiro[3.5]nonan-2-yl)pyridin-4-yl)oxy)phenyl)-2,4-dihydro-3H-1,2,4-triazol-3-one (15 mg, 12.30 yield) as a white solid. MS (m/z): 524.0 [M+H]⁺. 1H NMR (400 MHz, CDCl₃) δ 7.99 (d, J=9.1 Hz, 2H), 7.75 (d, J=5.9 Hz, 1H), 7.59 (s, 1H), 7.42-7.31 (i, 1H), 7.18-7.07 (m, 2H), 7.05-6.93 (i, 2H), 6.15 (t, J 5.6 Hz, 1H), 4.99 (s, 2H), 4.01 (s, 4H), 3.73-3.58 (m, 4H), 1.92-1.80 (n, 4H).

The synthesis procedure of Example (Compound) 673 was similar to the procedure of Example 672.

| Compound | | | | | |
|---|---|---|---|---|---|
| No. | Activi- | | Appearance | | MS (m/z) |
| Method | ty | Structure and Name | and Yield | ¹H NMR Data | [M + H]⁺ |
| Example 672 Method 139 | ++++ | 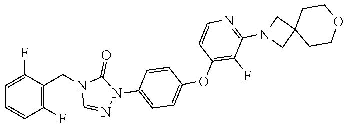 [SMILES: O=c1n(Cc2c(F)cccc2F)cnn1-c1ccc(Oc2ccnc(N3CC4(CCOCC4)C3)c2F)cc1] | white solid, yield: 12.3% | ¹H NMR (400 MHz, CDCl3) δ 7.99 (d, J = 9.1 Hz, 2H), 7.75 (d, J = 5.9 Hz, 1H), 7.59 (s, 1H), 7.42-7.31 (m, 1H), 7.18-7.07 (m, 2H), 7.05-6.93 (m, 2H), 6.15 (t, J = 5.6 Hz, 1H), 4.99 (s, 2H), 4.01 (s, 4H), 3.73-3.58 (m, 4H), 1.92-1.80 (m, 4H). | 524.0 |
| | | 4-(2,6-difluorobenzyl)-2-(4-((3- | | | |
| | | fluoro-2-(7-oxa-2-azaspiro[3.5] | | | |
| | | nonan-2-yl)pyridin-4-yl)oxy)phe- | | | |
| | | nyl)-2,4-dihydro-3H-1,2,4-triazol- | | | |
| | | 3-one | | | |
| Example 673 Method 139 | + | 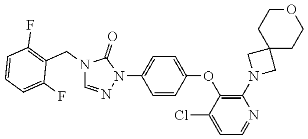 [SMILES: O=c1n(Cc2c(F)cccc2F)cnn1-c1ccc(Oc2c(Cl)ccnc2N2CC3(CCOCC3)C2)cc1] | white solid, yield: 35.1% | ¹H NMR (400 MHz, DMSO-d₆) δ 7.96 (s, 1H), 7.80 (dd, J = 13.2, 7.4 Hz, 3H), 7.50-7.33 (m, 1H), 7.03 (t, J = 8.1 Hz, 2H), 6.85 (d, J = 9.1 Hz, 2H), 6.47 (d, J = 5.7 Hz, 1H), 5.01 (s, 2H), 3.81 (s, 4H), 3.60-3.47 (m, 4H), 1.72- 1.63 (m, 4H). | 540.0 |
| | | 2-(4-((4-chloro-2-(7-oxa-2-azaspiro | | | |
| | | [3.5]nonan-2-yl)pyridin-3-yl) | | | |
| | | oxy)phenyl)-4-(2,6-difluoroben- | | | |
| | | zyl)-2,4-dihydro-3H-1,2,4-triazol- | | | |
| | | 3-one | | | |

### Example 674: Method 140

### 4-(2,6-difluorobenzyl)-2-(4-((2-(3-ethoxyazetidin-1-yl)-3,5-difluoropyridin-4-yl)oxy)pheny 1)-2,4-dihydro-3H-1,2,4-triazol-3-one

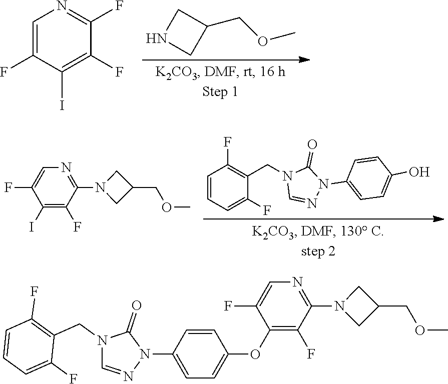

### Step 1: 3,5-difluoro-4-iodo-2-(3-(methoxymethyl)azetidin-1-yl)pyridine

2,3,5-trifluoro-4-iodopyridine (518 mg, 2 mmol), 3-(methoxymethyl)azetidine (220 mg, 2.2 mmol) and K₂CO₃ (552 mg, 4 mmol) were mixed in DMF (10 mL). The mixture was stirred at 25° C. for 16 hrs. The mixture was diluted with EA (50 mL) and washed with water (30 mL×3). The organic phase was concentrated and the residue was purified by column chromatography (PE: EA=2:1) to afford the titled product as a yellow solid (450 mg, 66.2% yield). MS (m/z): 341.1 [M+H]⁺.

### Step 2: 4-(2,6-difluorobenzyl)-2-(4-((2-(3-ethoxyazetidin-1-yl)-3,5-difluoropyridin-4-yl)oxy)phenyl)-2,4-dihydro-3H-1,2,4-triazol-3-one

A mixture solution of 3,5-difluoro-4-iodo-2-(3-(methoxymethyl)azetidin-1-yl)pyridine(263 mg, 0.77 mmol), 4-(2,6-difluorobenzyl)-2-(4-hydroxyphenyl)-2,4-dihydro-3H-1,2,4-triazol-3-one (235 mg, 0.77 mmol) and dipotassium carbonate (160 mg, 1.16 mmol) in DMF (10 mL) was stirred at 130° C. for 16 hrs. The mixture was diluted with EA (30 mL) and washed with water (30 mL×3). The organic phase was concentrated and the residue was purified by pre-TLC (PE: EA=2:1) to afford the titled product as a yellow solid (8.3 mg, 2% yield). MS (m/z): 516.2 [M+H]⁺, RT: 1.529 min.

| Compound | | | Appear- | | |
|---|---|---|---|---|---|
| No. | Activi- | | ance | ¹H NMR | MS (m/z) |
| Method | ty | Structure and Name | and Yield | Data | [M + H]⁺ |
| Example 674 Method 140 | ++++ | 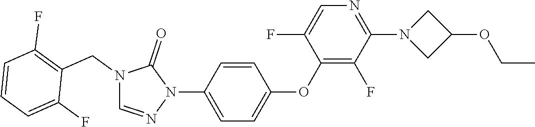 [SMILES: CCOC1CN(c2ncc(F)c(Oc3ccc(-n4ncn(Cc5c(F)cccc5F)c4=O)cc3)c2F)C1] | yellow solid, yield: 2% | NA | 516.2 |
| | | 4-(2,6-difluorobenzyl)-2-(4-((2-(3- | | | |
| | | ethoxyazetidin-1-yl)-3,5-diflu- | | | |
| | | oropyridin-4-yl)oxy)phenyl)-2,4- | | | |
| | | dihydro-3H-1,2,4-triazol-3-one | | | |

### Example 675: Method 141

### 4-(4-(4-(2,6-difluorobenzyl)-5-oxo-4,5-dihydro-1H-1,2,4-triazol-1-yl)-2-fluorophenoxy)-3-(methylamino)benzontrile

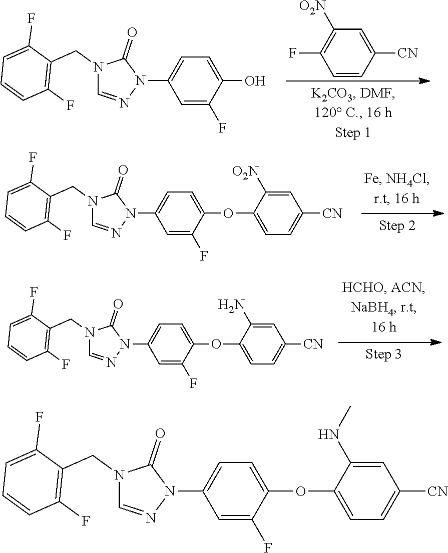 [SMILES: O=c1n(Cc2c(F)cccc2F)cnn1-c1ccc(O)c(F)c1 ; N#Cc1ccc(F)c([N+](=O)[O-])c1 ; N#Cc1ccc(Oc2ccc(-n3ncn(Cc4c(F)cccc4F)c3=O)cc2F)c([N+](=O)[O-])c1 ; N#Cc1ccc(Oc2ccc(-n3ncn(Cc4c(F)cccc4F)c3=O)cc2F)c(N)c1 ; CNc1cc(C#N)ccc1Oc1ccc(-n2ncn(Cc3c(F)cccc3F)c2=O)cc1F]

### Step 1: 4-(4-(4-(2,6-difluorobenzyl)-5-oxo-4,5-dihydro-1H-1,2,4-triazol-1-yl)-2-fluorophenoxy)-3-nitrobenzonitrile

To a stirred solution of 4-[(2,6-difluorophenyl)methyl]-2-(3-fluoro-4-hydroxyphenyl)-1,2,4-triazol-3-one (1.0 g, 3.11 mmol), 4-fluoro-3-nitrobenzonitrile (0.51 g, 3.11 mmol) in DMF (15 mL) was added K₂CO₃ (1.3 g, 9.33 mmol) under nitrogen. The reaction mixture was stirred at 120° C. for 16 h. The mixture was allowed to cool down to rt. The resulted mixture was diluted with water (1000 mL). The resulted mixture was extracted with EtOAc (3×80 mL). The combined organic layers were washed with brine (100 mL), dried over anhydrous Na₂SO₄, and after filtration, the filtrate was concentrated under reduced pressure. The residue was purified by silica gel column chromatography, eluted with (PE:EtOAc=1:1) to afford 4-(4-(4-(2,6-difluorobenzyl)-5-oxo-4,5-dihydro-1H-1,2,4-triazol-1-yl)-2-fluorophenoxy)-3-nitrobenzonitrile (0.40 g, 28.68% yield) as a brown yellow solid. MS (m/z):468.1 [M+H]⁺.

### Step 2: 3-amino-4-(4-(4-(2,6-difluorobenzyl)-5-oxo-4,5-dihydro-1H-1,2,4-triazol-1-yl)-2-fluorophenoxy)benzonitrile

To a stirred solution of 4-(4-{4-[(2,6-difluorophenyl)methyl]-5-oxo-1,2,4-triazol-1-yl}-2-fluorophenoxy)-3-nitrobenzonitrile (730 mg, 1.56 mmol) and NH₄Cl (501 mg, 9.37 mmol) in EtOH (20 mL) was added Fe (872 mg, 15.62 mmol) under nitrogen. The reaction mixture was stirred at 25° C. for 16 h. The solution was filtered and the filtrate was collected. The resulted mixture was diluted with water (40 mL). The resulted mixture was extracted with EtOAc (3×40 mL). The combined organic layers were washed with brine (80 mL), dried over anhydrous Na₂SO₄. After filtration, the filtrate was concentrated under reduced pressure. The residue was purified by silica gel column chromatography, eluted with (DCM:MeOH=20:1) to afford 3-amino-4-(4-(4-(2,6-difluorobenzyl)-5-oxo-4,5-dihydro-1H-1,2,4-triazol-1-yl)-2-fluorophenoxy)benzonitrile (634 mg, 92.74% yield) as a yellow solid. MS (m/z): 438.1 [M+H]⁺.

### Step 3: 4-(4-(4-(2,6-difluorobenzyl)-5-oxo-4,5-dihydro-1H-1,2,4-triazol-1-yl)-2-fluorophenoxy)-3-(methylamino)benzonitrile

To a solution of 3-amino-4-(4-{4-[(2,6-difluorophenyl)methyl]-5-oxo-1,2,4-triazol-1-yl}-2-fluorophenoxy)benzonitrile (50 mg 0.11 mmol) and NaBH₄ (43 mg, 1.14 mmol) in ACN (5 mL) stirred under nitrogen at 25° C. was added a solution of formaldehyde (17 mg, 0.57 mmol) in ACN (1 mL) dropwise. The reaction mixture was stirred at 25° C. for 8 h. The resulted mixture was diluted with water (40 mL). The resulted mixture was extracted with EtOAc (3×40 mL). The combined organic layers were washed with brine (80 mL), dried over anhydrous Na₂SO₄, and after filtration, the filtrate was concentrated under reduced pressure. The residue was purified by silica gel column chromatography, eluted with (DCM:MeOH=20:1) to afford 4-(4-(4-(2,6-difluorobenzyl)-5-oxo-4,5-dihydro-1H-1,2,4-triazol-1-yl)-2-fluorophenoxy)-3-(methylamino)benzonitrile (5 mg, 10.2% yield) as a white solid. LC-MS (m/z): 452.1 [M+H]⁺. ¹H NMR (400 MHz, CDCl₃) δ 7.94 (dd, J=12.0, 2.5 Hz, 1H), 7.83-7.79 (m, 1H), 7.61 (s, 1H), 7.41-7.35 (m, 1H), 7.13 (t, J=8.8 Hz, 1H), 7.03-6.96 (m, 2H), 6.87 (dt, J=6.0, 1.8 Hz, 2H), 6.57 (d, J=8.0 Hz, 1H), 4.99 (s, 2H), 2.93 (s, 3H).

The synthesis procedure of Examples (Compounds) 676-679 was similar to the procedure of Example 675.

| | | | | | MS |
|---|---|---|---|---|---|
| Compound | | | | | (m/z) |
| No. | Activi- | | Appearance | | [M + |
| Method | ty | Structure and Name | and Yield | ¹H NMR Data | H]⁺ |
| Example 675 Method 141 | +++ | 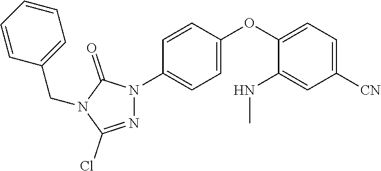 [SMILES: CNc1cc(C#N)ccc1Oc1ccc(-n2nc(Cl)n(Cc3ccccc3)c2=O)cc1] | yellow solid, yield: 28% | ¹H NMR (400 MHz, DMSO-d₆) δ ppm 7.85 (m, 2H), 7.40 (m, 2H), 7.35 (m, 3H), 7.16 (m, 2H), 6.96 (dd, J = 5.8, 1.9 Hz, 2H), 6.77 (d, J = 8.6 Hz, 1H), 6.02 (d, J = 5.1 Hz, 1H), 4.94 (s, 2H), 2.75 (d, J = 4.9 Hz, 3H). | 432.0 |
| | | 4-benzyl-5-chloro-2-(4-(2-(meth- | | | |
| | | ylamino)phenoxy)phenyl)-2,4-di- | | | |
| | | hydro-3H-1,2,4-triazol-3-one | | | |
| Example 676 Method 141 | ++++ | 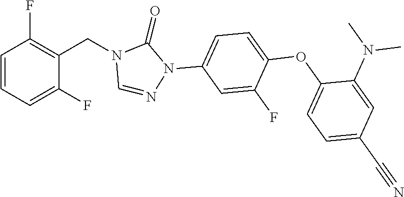 [SMILES: CN(C)c1cc(C#N)ccc1Oc1ccc(-n2ncn(Cc3c(F)cccc3F)c2=O)cc1F] | white solid, yield: 8.5% | ¹H NMR (400 MHz, CDCl₃) δ 7.97 (dd, J = 12.1, 2.4 Hz, 1H), 7.83 (d, J = 8.9 Hz, 1H), 7.61 (s, 1H), 7.38 (ddd, J = 15.0, 8.4, 6.5 Hz, 2H), 7.24-7.09 (m, 2H), 7.00 (dd, J = 13.3, 5.8 Hz, 2H), 6.72 (d, J = 8.4 Hz, 1H), 4.99 (s, 2H), 2.97 (s, 6H). | 466.1 |
| | | 4-(4-(4-(2,6-difluorobenzyl)-5-oxo- | | | |
| | | 4,5-dihydro-1H-1,2,4-triazol- | | | |
| | | 1-yl)-2-fluorophenoxy)-3-(dimeth- | | | |
| | | ylamino)benzonitrile | | | |
| Example 677 Method 141 | +++ | 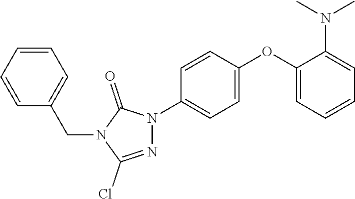 [SMILES: CN(C)c1ccccc1Oc1ccc(-n2nc(Cl)n(Cc3ccccc3)c2=O)cc1] | yellow solid, yield: 18% | ¹H NMR (400 MHz, CDCl₃) δ ppm 7.98 (m, 2H), 7.77 (dd, J = 8.2, 1.5 Hz, 1H), 7.44 (m, 1H), 7.37 (dd, J = 3.3, 1.7 Hz, 4H), 7.30 (m, 4H), 6.97 (dd, J = 8.4, 1.3 Hz, 1H), 4.97 (s, 2H), 3.32 (s, 6H). | 420.6 |
| | | 4-benzyl-5-chloro-2-(4-(2-(dimeth- | | | |
| | | ylamino)phenoxy)phenyl)-2,4- | | | |
| | | dihydro-3H-1,2,4-triazol-3-one | | | |
| Example 678 Method 139 | ++++ | 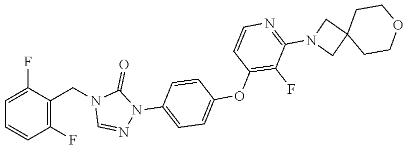 [SMILES: O=c1n(Cc2c(F)cccc2F)cnn1-c1ccc(Oc2ccnc(N3CC4(CCOCC4)C3)c2F)cc1] | white solid, yield: 12.3% | ¹H NMR (400 MHz, CDCl3) δ 7.99 (d, J = 9.1 Hz, 2H), 7.75 (d, J = 5.9 Hz, 1H), 7.59 (s, 1H), 7.42-7.31 (m, 1H), 7.18-7.07 (m, 2H), 7.05-6.93 (m, 2H), 6.15 (t, J = 5.6 Hz, 1H), 4.99 (s, 2H), 4.01 (s, 4H), 3.73-3.58 (m, 4H), 1.92-1.80 (m, 4H). | 524.0 |
| | | 4-(2,6-difluorobenzyl)-2-(4-((3- | | | |
| | | fluoro-2-(7-oxa-2-azaspiro[3.5] | | | |
| | | nonan-2-yl)pyridin-4-yl)oxy)phe- | | | |
| | | nyl)-2,4-dihydro-3H-1,2,4-triazol- | | | |
| | | 3-one | | | |
| Example 679 Method 141 | ++++ | 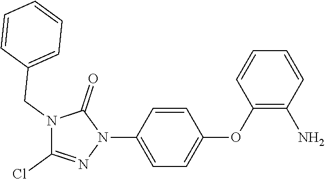 [SMILES: Nc1ccccc1Oc1ccc(-n2nc(Cl)n(Cc3ccccc3)c2=O)cc1] | yellow solid, yield: 25% | ¹H NMR (400 MHz, DMSO-d₆) δ ppm 7.73 (m, 2H), 7.38 (dt, J = 16.7, 7.8 Hz, 5H), 6.96 (m, 3H), 6.81 (dd, J = 8.0, 1.41 Hz, 2H), 6.56 (td, J = 7.8, 1.6 Hz, 1H), 4.95 (s, 1H), 4.93 (s, 2H). | 393.1 |
| | | 2-(4-(2-aminophenoxy)phenyl)- | | | |
| | | 4-benzyl-5-chloro-2,4-dihydro-3H- | | | |
| | | 1,2,4-tirazol-3-one | | | |

### Example 680: Method 142

### 2-(4-((2-(6-cyclopropyl-2,6-diazaspiro[3.3]heptan-2-yl)-5-fluoropyridin-4-yl)oxy)-3-fluoro phenyl)-4-(2,6-difluorobenzyl)-2,4-dihydro-3H-1,2,4-triazol-3-one

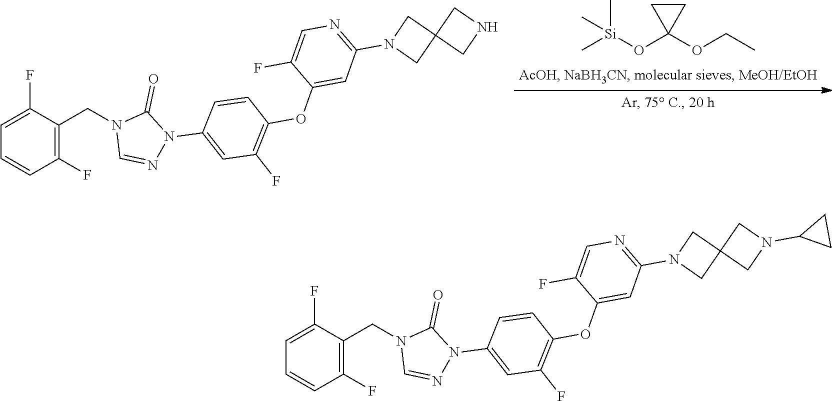 [SMILES: O=c1n(Cc2c(F)cccc2F)cnn1-c1ccc(Oc2cc(N3CC4(CNC4)C3)ncc2F)c(F)c1 ; CCOC1(O[Si](C)(C)C)CC1 ; O=c1n(Cc2c(F)cccc2F)cnn1-c1ccc(Oc2cc(N3CC4(C3)CN(C3CC3)C4)ncc2F)c(F)c1]

### 2-(4-((2-(6-cyclopropyl-2,6-diazaspiro[3.3]heptan-2-yl)-5-fluoropyridin-4-yl)oxy)-3-fluoro phenyl)-4-(2,6-difluorobenzyl)-2,4-dihydro-3H-1,2,4-triazol-3-one

To a stirred solution of 4-(2,6-difluorobenzyl)-2-(3-fluoro-4-((5-fluoro-2-(2,6-diazaspiro[3.3]heptan-2-yl)pyridin-4-yl)oxy)phenyl)-2,4-dihydro-3H-1,2,4-triazol-3-one (100 mg, 0.20 mmol) in MeOH (3 mL) and EtOH (3 mL) were added (1-ethoxycyclopropoxy)trimethylsilane (204 mg, 1.17 mmol), acetic acid (117 mg, 1.95 mmol), NaBH₃CN (61 mg, 0.98 mmol) and molecular sieves (50 mg) at rt. The resulted mixture was stirred for additional 20 h at 75° C. under Ar. The mixture was allowed to cool down to rt. The resulted mixture was diluted with water (100 mL). The resulted mixture was extracted with EtOAc (3×80 mL). The combined organic layers were washed with brine (100 mL), dried over anhydrous Na₂SO₄. After filtration, the filtrate was concentrated under reduced pressure. The residue was purified by silica gel column chromatography, eluted with (PE:EtOAc=1:1) to afford the titled compound (54 mg, 50.1% yield) as a white solid. LC-MS (m/z): 553.2 [M+H]⁺. ¹H NMR (400 MHz, DMSO-d₆) δ 8.40 (s, 1H), 8.11 (d, J=3.0 Hz, 1H), 7.92 (dd, J=12.4, 2.5 Hz, 1H), 7.80-7.70 (m, 1H), 7.52-7.38 (m, 2H), 7.20-7.09 (m, 2H), 5.71 (d, J=5.6 Hz, 1H), 5.00 (s, 2H), 3.85 (s, 4H), 3.33 (s, 4H), 1.81 (s, 1H), 0.30 (d, J=4.8 Hz, 2H), 0.18 (s, 2H).

| | | | Appear- | | MS |
|---|---|---|---|---|---|
| Compound | | | ance | | (m/z) |
| No. | Activi- | | and | | [M + |
| Method | ty | Structure and Name | Yield | ¹H NMR Data | H]⁺ |
| Example 680 Method 142 | ++++ | 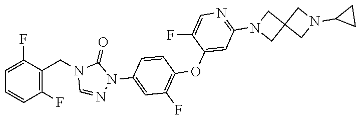 [SMILES: O=c1n(Cc2c(F)cccc2F)cnn1-c1ccc(Oc2cc(N3CC4(C3)CN(C3CC3)C4)ncc2F)c(F)c1] 2-(4-((2-(6-cyclopropyl-2,6-diaza- spiro[3.3]heptan-2-yl)-5-fluoro- | white solid, yield: 50.1% | ¹H NMR (400 MHz, DMSO-d₆) δ 8.40 (s, 1H), 8.11 (d, J = 3.0 Hz, 1H), 7.92 (dd, J = 12.4, 2.5 Hz, 1H), 7.80-7.70 (m, 1H), 7.52-7.38 (m, 2H), 7.20-7.09 (m, 2H), 5.71 (d, J = 5.6 Hz, 1H), 5.00 (s, 2H), 3.85 (s, 4H), 3.33 (s, 4H), 1.81 (s, 1H), 0.30 (d, J = 4.8 Hz, 2H), 0.18 (s, 2H). | 553.2 |
| | | pyridin-4-yl)oxy)-3-fluorophenyl)- | | | |
| | | 4-(2,6-difluorobenzyl)-2,4-di- | | | |
| | | hydro-3H-1,2,4-triazol-3-one | | | |

### Example 681: Method 143

### 4-benzyl-5-chloro-2-(4-(2,3-dihydro-4H-benzo[b][1,4]oxazin-4-yl)phenyl)-2,4-dihydro-3H-1,2,4-triazol-3-one

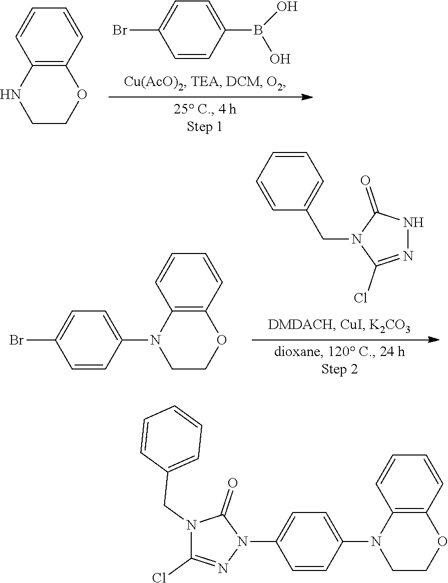

### Step 1: 4-(4-bromophenyl)-3,4-dihydro-2H-benzo[b][1,4]oxazine

A solution of 3,4-dihydro-2H-benzo[b][1,4]oxazine (300 mg, 2.22 mmol), (4-bromophenyl)boronic acid (1337 mg 6.66 mmol), Cu(OAc)₂ (121 mg 0.67 mmol), TEA (674 mg 6.66 mmol) in DCM (20 mL) was stirred at 25° C. for 4 h under O₂. The resulted mixture was diluted with water (30 mL). The resulted mixture was extracted with DCM (3×20 mL). The combined organic layers were washed with brine (30 mL), dried over anhydrous Na₂SO₄, and after filtration, the filtrate was concentrated under reduced pressure. The residue was purified by silica gel column chromatography, eluted with (PE:EtOAc=10:1) to afford 4-(4-bromophenyl)-3,4-dihydro-2H-benzo[b][1,4]oxazine (250 mg, 38.8% yield) as a colorless oil. MS (m/z): 289.9, 291.7 [M+H]⁺.

### Step 2: 4-benzyl-5-chloro-2-(4-(2,3-dihydro-4H-benzo[b][1,4]oxazin-4-yl)phenyl)-2,4-dihydro-3H-1,2,4-triazol-3-one

A mixture solution of 4-(4-bromophenyl)-3,4-dihydro-2H-benzo[b][1,4]oxazine (104.5 mg, 0.5 mmol), 4-benzyl-5-chloro-2,4-dihydro-3H-1,2,4-triazol-3-one (159.5 mg, 0.55 mmol), copper(I) iodide (9.5 mg, 0.05 mmol), DMDACH (14.2 mg, 0.1 mol) and K₂CO₃ (138 mg, 1 mmol) in dioxane (5 mL) was stirred at 120° C. for 24 hrs. After cooling, the mixture was diluted with water (10 mL), extracted three times with ethyl acetate (40 mL). Organic layers were combined, and solvent was removed under vacuum and the crude was purified through silica gel chromatography (PE/EA=1:1) to give 4-benzyl-5-chloro-2-(4-(2,3-dihydro-4H-benzo[b][1,4]oxazin-4-yl)phenyl)-2,4-dihydro-3H-1,2,4-triazol-3-one (30 mg, 28.6% yield) as a white solid. LC-MS (m/z): 418.8 [M+H]⁺. ¹H NMR (400 MHz, DMSO-d₆) δ 7.85-7.76 (m, 2H), 7.42-7.30 (m, 7H), 6.87-6.81 (m, 2H), 6.75-6.68 (m, 2H), 4.95 (s, 2H), 4.30-4.18 (m, 2H), 3.77-3.64 (m, 2H).

| Compound | | | Appear- | | |
|---|---|---|---|---|---|
| No. | Activi- | | ance | | MS (m/z) |
| Method | ty | Structure and Name | and Yield | ¹H NMR Data | [M + H]⁺ |
| Example 681 Method 143 | +++ | 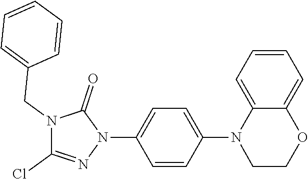 [SMILES: O=c1n(-c2ccc(N3CCOc4ccccc43)cc2)nc(Cl)n1Cc1ccccc1] | colorless oil, yield: 38.8% | ¹H NMR (400 MHz, DMSO-d₆) δ 7.85-7.76 (m, 2H), 7.42-7.30 (m, 7H), 6.87-6.81 (m, 2H), 6.75-6.68 (m, 2H), 4.95 (s, 2H), 4.30-4.18 (m, 2H), 3.77-3.64 (m, 2H). | 418.8 |
| | | 4-benzyl-5-chloro-2-(4-(2,3-dihy- | | | |
| | | dro-4H-benzo[b][1,4]oxazin-4- | | | |
| | | yl)phenyl)-2,4-dihydro-3H-1,2,4- | | | |
| | | triazol-3-one | | | |

### Example 682: Method 144

### 3-(3-(4-(4-(2,6-difluorobenzyl)-5-oxo-4,5-dihydro-1H-1,2,4-triazol-1-yl)benzyl)-2-oxopyridin-1(2H)-yl)cyclobutane-1-carbonitrile

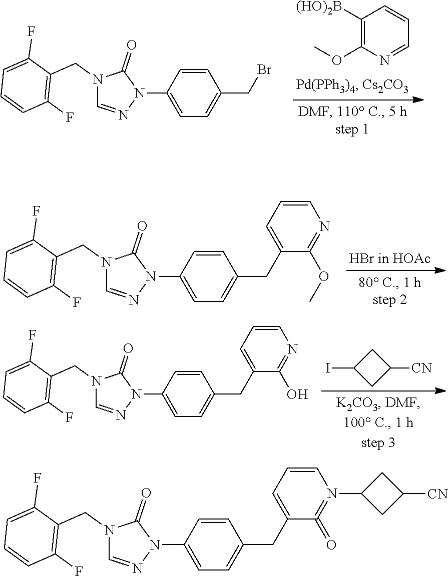

### Step 1: 4-(2,6-difluorobenzyl)-2-(4-((2-methoxypyridin-3-yl)methyl)phenyl)-2,4-dihydro-3H-1,2,4-triazol-3-one

Cs₂CO₃ (771 mg, 2.37 mmol) and Pd(PPh₃)₄(91 mg, 0.079 mmol) were added to a solution of 2-[4-(bromomethyl)phenyl]-4-[(2,6-difluorophenyl)methyl]-1,2,4-triazol-3-one(300 mg, 0.79 mmol), and (2-methoxypyridin-3-yl)boranediol(144 mg, 0.95 mmol) in DMF(10 mL). The mixture was heated to 110° C. for 5 h, then cooled to rt, the solvent was removed under vacuum and the crude was purified through silica gel chromatography (PE: EA=3:1) to give the desired product as yellow solid (140 mg, 43.4%). LCMS (m/z): 409.1 [M+H]⁺, RT: 1.556 min.

### Step 2. 4-(2,6-difluorobenzyl)-2-(4-((2-hydroxypyridin-3-yl)methyl)phenyl)-2,4-dihydro-3H-1,2,4-triazol-3-one

A solution of 4-[(2,6-difluorophenyl)methyl]-2-{4-[(2-methoxypyridin-3-yl)methyl]phenyl}-1,2,4-triazol-3-one(10 mg, 0.025 mmol) in HBr in AcOH(2 mL) was heated to 80° C. for 1h, cooled to rt, and the solvent was removed under vacuum to give the desired product as a yellow solid (8 mg, 82.9% yield). LCMS (m/z): 395.1 [M+H]⁺, RT: 1.346 min.

### Step 3. Preparation of 3-(3-(4-(4-(2,6-difluorobenzyl)-5-oxo-4,5-dihydro-1H-1,2,4-triazol-1-yl)benzyl)-2-oxopyridin-1(2H)-yl)cyclobutane-1-carbonitrile

3-iodocyclobutane-1-carbonitrile(63 mg, 0.30 mmol) and K₂CO₃ (105 mg, 0.76 mmol) were added to a solution of 4-[(2,6-difluorophenyl)methyl]-2-{4-[(2-hydroxypyridin-3-yl)methyl]phenyl}-1,2,4-triazol-3-one(100 mg, 0.25 mmol) in DMF (10 mL). The mixture was heated to 100° C. for 1h, then cooled to r.t., and the solvent was removed under vacuum to give a crude product which was purified by Prep-HPLC (column-Gemini —C18 150×21.2 mm, Sum; Mobile phase: ACN-H₂O (0.1% FA), 5%-20%) to afford the desired product as a white solid (2.5 mg, 2.1% yield). LCMS (m/z): 474.1[M+H]⁺, RT: 1.212 min. ¹H NMR (400 MHz, CDCl₃) δ 7.87 (d, J=8.5 Hz, 2H), 7.56 (s, 1H), 7.40-7.33 (m, 1H), 7.28 (s, 1H), 7.23 (d, J=7.0 Hz, 1H), 7.08 (d, J=6.9 Hz, 1H), 6.98 (m, 3H), 6.20-6.05 (m, 1H), 5.26-5.16 (m, 1H), 4.97 (s, 2H), 3.83 (s, 2H), 3.27 (d, J=3.8 Hz, 1H), 3.04-2.83 (m, 4H).

The synthesis procedure of Examples (Compounds) 683-684 were similar to the procedure of Example 682.

| Com- | | | Appear- | | MS |
|---|---|---|---|---|---|
| pound | | | ance | | (m/z) |
| No. | Activi- | | and | | [M + |
| Method | ty | Structure and Name | Yield | ¹H NMR Data | H]⁺ |
| Example 682 Method 144 | +++ | 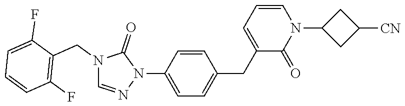 [SMILES: N#CC1CC(n2cccc(Cc3ccc(-n4ncn(Cc5c(F)cccc5F)c4=O)cc3)c2=O)C1] 3-(3-(4-(4-(2,6-difluorobenzyl)- 5-oxo-4,5-dihydro-1H-1,2,4-tri- | white solid, yield: 3.2% | ¹H NMR (400 MHz, CDCl₃) δ 7.87 (d, J = 8.5 Hz, 2H), 7.56 (s, 1H), 7.40-7.33 (m, 1H), 7.28 (s, 1H), 7.23 (d, J = 7.0 Hz, 1H), 7.08 (d, J = 6.9 Hz, 1H), 6.98 (m, 3H), 6.20-6.05 (m, 1H), 5.26-5.16 (m, 1H), 4.97 (s, 2H), 3.83 (s, 2H), 3.27 (d, J = 3.8 Hz, 1H), 3.04-2.83 (m, 4H). | 474.1 |
| | | azol-1-yl)benzyl)-2-oxopyridin-1 | | | |
| | | (2H)-yl)cyclobutane-1-carbonitrile | | | |
| Example 683 Method 144 | ++++ | 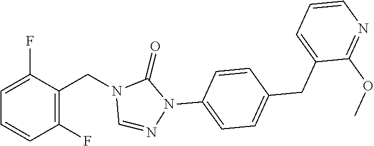 [SMILES: COc1ncccc1Cc1ccc(-n2ncn(Cc3c(F)cccc3F)c2=O)cc1] | white solid, yield: 43.4% | ¹H NMR (400 MHz, DMSO-d₆) δ 8.27 (s, 1H), 8.03 (m, 1H), 7.75 (d, J = 8.6 Hz, 2H), 7.48 (d, J = 6.3 Hz, 2H), 7.27 (d, J = 8.7 Hz, 2H), 7.16 (m, 2H), 6.92 (m, 1H), 4.96 (s, 2H), 3.87 (s, 2H), 3.85 (s, 3H). | 409.1 |
| | | 4-(2,6-difluorobenzyl)-2-(4-((2- | | | |
| | | methoxypyridin-3-yl)methyl)phe- | | | |
| | | nyl)-2,4-dihydro-3H-1,2,4-triazol- | | | |
| | | 3-one | | | |
| Example 684 Method 144 | +++ | 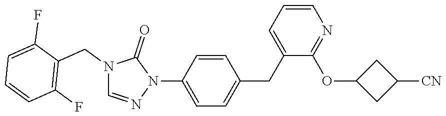 [SMILES: N#CC1CC(Oc2ncccc2Cc2ccc(-n3ncn(Cc4c(F)cccc4F)c3=O)cc2)C1] 3-((3-(4-(4-(2,6-difluorobenzyl)- | white solid, yield: 2.2% | ¹H NMR (400 MHz, CDCl₃) δ 7.98 (m, 1H), 7.86 (d, J = 8.6 Hz, 2H), 7.57 (s, 1H), 7.39-7.31 (m, 2H), 7.21 (d, J = 8.6 Hz, 2H), 7.00-6.95 (m, 2H), 6.82 (m, 1H), 5.48 (m, 1H), 4.97 (s, 2H), 3.88 (s, 2H), 3.13-3.05 (m, 1H), 2.88-2.81 (m, 2H), 2.50-2.43 (m, 2H). | 474.1 |
| | | 5-oxo-4,5-dihydro-1H-1,2,4-tri- | | | |
| | | azol-1-yl)benzyl)pyridin-2-yl)oxy) | | | |
| | | cyclobutane-1-carbonitrile | | | |

### Example 2. Biological Assay

Compounds 1-702 of the disclosure were tested for biological activity, following the experimental procedures described below.

### Materials:

Cell line: HT-29 (ATCC® HTB-38™)
Culture medium: McCOY's 5A, Gibco, Cat No. 16600-082
FBS, Gibco, Cat No. 10099-141 C
Trypsin: Gibco, Cat No. 25200-056
DMSO: Sigma, Cat No. 67-68-5, 1L
Assay plate: Corning #3903
Compound dilution plate: Corning #3357
Inducers: TNFα, GenScript, Cat No. Z01001-50,
SmacM, Cat. No., HY-15989, MedChemExpress (MCE)
Z_VAD FMK, TargetMol, T6013
Cell Titer-Glo® Luminescent Cell Viability Assay Kit: Promega, Cat No. G7573
EnVision: PerkinElmer, 2105-0010

### Cell Seeding

1. HT-29 cells were checked every day to make sure that they were healthy and growing as expected. They were subjected to sub-culturing when they were approximately 80% confluent.
2. The culture medium, McCOY's 5A medium (Gibco, Cat No. 16600-082) with 10% fetal bovine serum or FBS (Gibco, Cat No. 10099-141C), was pre-warmed in a 37° C. water bath for at least 30 min.
3. When the cells had reached a desired level of confluency of 80% in a T75 flask, the medium was aspirated, and the cells were washed with warm phosphate buffered saline or PBS two times.
4. 2-3 ml fresh warm trypsin (Gibco, Cat No. 25200-056) solution was added to the washed cells. The flask with the cells was transferred to a 37° C. incubator.
5. After 5 minutes, the side of the flask was tapped, and the flask was examined under a microscope for detachment of the cells to the flask. If necessary, the cells were kept in the incubator for an additional 5-10 minutes, with occasional tapping, until lifting was complete.
6. The trypsin reaction was neutralized by transferring 6-9 ml cell culture medium to sterile 15 ml conical tubes, and by centrifuging the cell culture at 300×g for 7 minutes to pellet the cells (supernatant decanted).
7. The cells were resuspended in fresh cell culture medium and the cell counting was performed using a hemocytometer.
8. 100 μl of the resuspended cell culture medium containing ˜5,000 cells were transferred into each well of the sterile 96-well cell culture plate (Corning 3903) and cultured overnight at 37° C. with 5% CO2.

### Compound Titration and Treatment:

1. All test compounds were dissolved in DMSO (Dimethyl sulfoxide) to create a 20 mM stock.
2. 3 μl of 20 mM stock of each compound was mixed with 27 μl DMSO, and the compound solution was further diluted at a titration ratio of 1:3 (20 μl compound solution+40 μl DMSO) till the 10 points end.
3. All culture medium was removed from assay plates filled with HT-29 cell cultures. The cells were then washed with 1 PBS, and resuspsended in fresh, FBS-free McCOY's 5A medium containing a cocktail of TNF-α (10 ng/ml), a SMAC mimetic compound (6 μM) and Z-VAD-fluoromethylketone or zVAD-FMK (10 μM) to stimulate the HT-29 cells to increase RIP1 kinase levels and necroptosis.
4. 0.5 μL of the diluted compound solution was added to the corresponding 96-well assay plates.
5. The assay plates were incubated for 20 hours at 37° C. with 5% CO2.

### Cell Viability Detection

1. The CellTiter-Glo® Luminescent Cell Viability Assay was employed to detect the ATP levels of viable HT-29 cells.
2. The CellTiter-Glo® buffer and the lyophilized substrate were equilibrated to room temperature prior to use.
3. The CellTiter-Glo® substrate was resuspended with CellTiter-Glo® buffer, then mixed by gently vortexing to obtain a homogeneous solution.
4. 20 μl of the enzyme/substrate mixture was transferred by multi-channel pipetting into 96-well assay plates.
5. The assay plates were placed on an orbital shaker and the contents were shaken for 3 minutes to induce cell lysis.
6. The assay plates were incubated at room temperature for 10 minutes to stabilize the luminescent signal.
7. The luminescence signals were read and recorded with EnVision.
8. The geometric mean IC50 values were calculated from 10 points response dose with duplicates.

IC₅₀ value ranges of Compounds 1-702 are provided as follows: ++++=IC₅₀<0.1 μM; +++=0.1 μM≤IC₅₀<1 μM; ++=1 μM≤IC₅₀≤10 μM; +=IC₅₀>10 μM.

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

The invention claimed is:

1. A compound of the following structural Formula I: 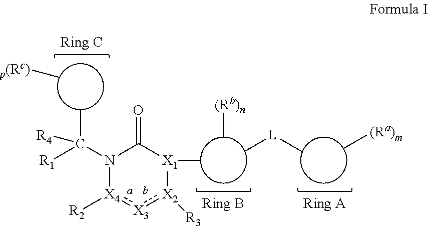
a tautomer thereof, a solvate or stereoisomer of the compound or the tautomer, or a pharmaceutically acceptable salt of the foregoing, wherein:
X₁ is C or N; X₂ is C or N; X₃ is C, N, or absent; X₄ is C or N;
Ring A is phenyl, 5- to 9-membered heteroaryl, 5- to 6-membered cycloalkyl, or 5- to 9-membered heterocyclyl;
Ring B is phenyl, 5- to 9-membered heteroaryl, 5- to 6-membered cycloalkyl, or 4- to 8-membered heterocyclyl;
Ring C is phenyl, 5- to 6-membered heteroaryl, 5- to 6-membered cycloalkyl, or 5- to 6-membered heterocyclyl;
bond a and bond b are each independently selected from a single bond and a double bond, provided that bond a and bond b cannot be double bond at the same time, and when X₃ is absent, the bond between X₂ and X₄ is a single bond or a double bond;
R^{a}, for each occurrence, is independently selected from halogen, cyano, ═O, NO₂, optionally substituted C₁ to C₆ alkyl, optionally substituted C₂ to C₆ alkenyl, optionally substituted C₂ to C₆ alkynyl, optionally substituted acyl, optionally substituted 3 to 10-membered cycloalkyl, optionally substituted 3 to 10-membered heterocyclyl, optionally substituted phenyl, optionally substituted 5 to 10-membered heteroaryl, optionally substituted nitrogen, and optionally substituted oxygen;
R^{b}, for each occurrence, is independently selected from halogen, CN, ═O, and C₁-C₄ alkyl optionally substituted by 1 to 3 groups of halogen;
R^{c}, for each occurrence, is independently, is selected from halogen, CN, C₁-C₆ alkyl, OR^{s1}, and —C(═O)OR^{s1};
R₁ is H, R₂ is selected from H, halogen, CN, OR^{s1}, —NR^{p1}R^{q1}, ═O, and C₁ to C₃ alkyl optionally substituted by 1 to 3 groups selected from halogen, or R₁ and R₂ join to form a 5- to 6-membered carbocycle or heterocycle optionally substituted by 1 to 3 groups selected from halogen and C₁ to C₃ alkyl optionally substituted by 1 to 3 groups selected from halogen;
R₃ is selected from H and ═O, provided that when R₃ is ═O, X₂ is C;
R₄ is selected from H and C₁ to C₃ alkyl;
L is selected from —NR^{x}—, —(CH₂)ᵤO(CH₂)ᵤ—, —(CH₂)ᵤS(═O)_{w}—(CH₂)ᵤ—, —S(═O)_{w}(═NR^{x})—, —NR^{x}S(═O)_{w}—, —S(═O)_{w}(NR^{x})—, —C(═O)—, and C₁-C₃ alkylene, wherein the C₁-C₃ alkylene of L is optionally substituted by 1 to 2 groups selected from OH, C₁-C₃ alkyl, and ═CHR^{x}, wherein the C₁-C₃ alkyl of the C₁-C₃ alkylene of L optionally join to form a C₃-C₄ cycloalkyl; wherein
R^{p1} and R^{q1}, for each occurrence, are each independently selected from hydrogen and C₁-C₄ alkyl optionally substituted with 1 to 3 groups selected from halogen, CN, and OH;
R^{s1}, for each occurrence, is independently selected from hydrogen and C₁-C₄ alkyl optionally substituted with 1 to 3 groups selected from halogen, CN, and OH; and
R^{x} is selected from H and C₁-C₄ alkyl;
m and p are each an integer independently selected from 0, 1, 2, 3, and 4;
n is an integer selected from 0, 1, and 2;
w, for each occurrence, is an integer independently selected from 0, 1, and 2;
u, for each occurrence, is an integer independently selected from 0, 1, and 2;
provided that the compounds is not: 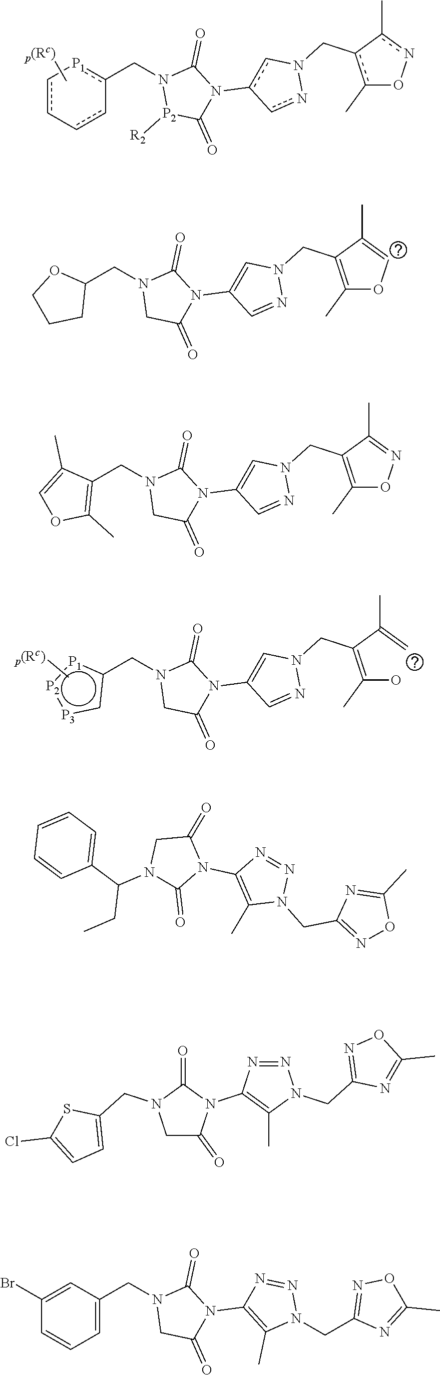 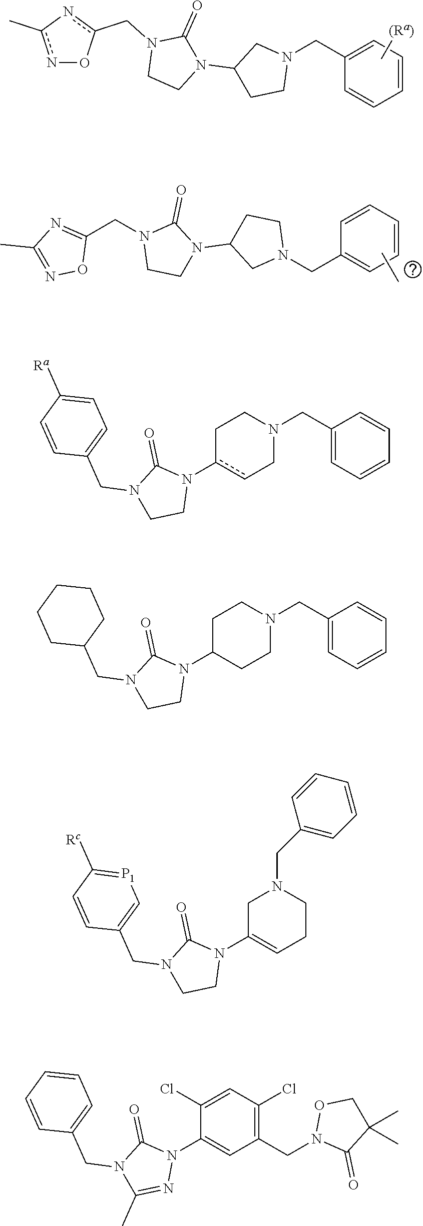 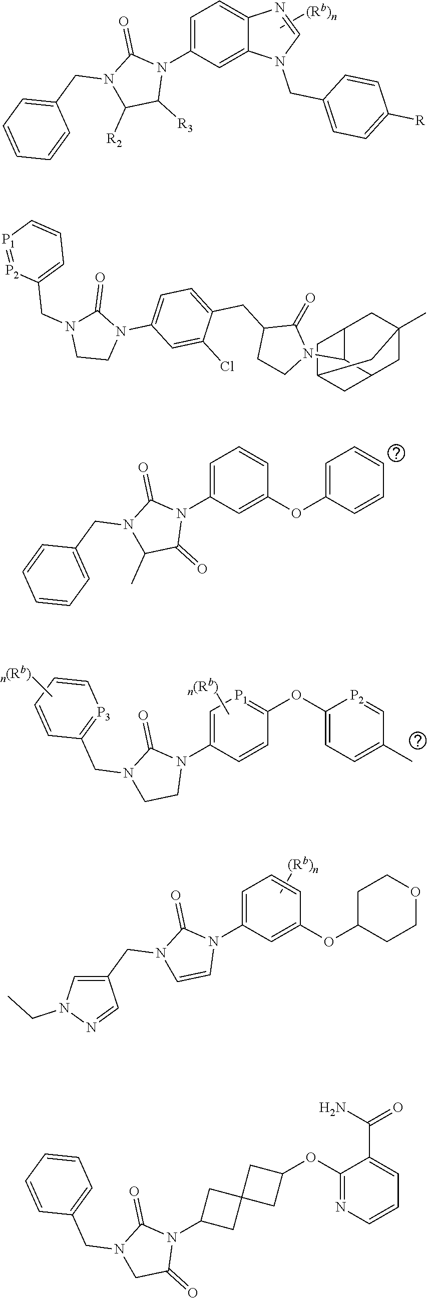 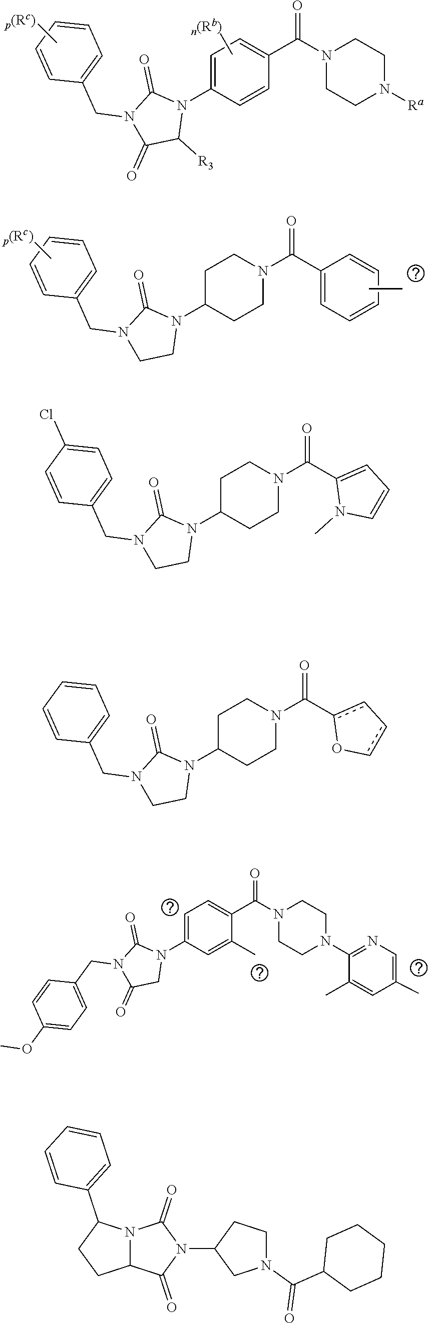 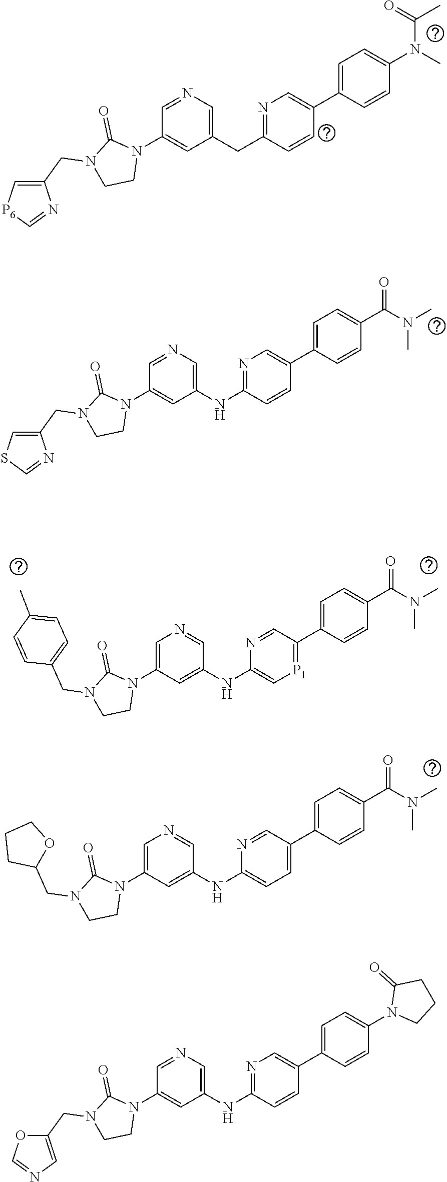 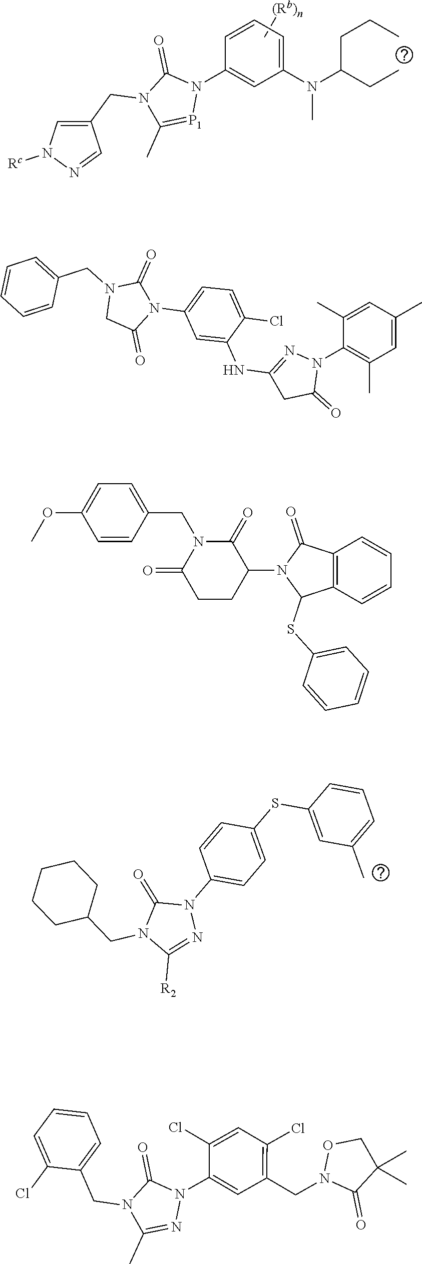 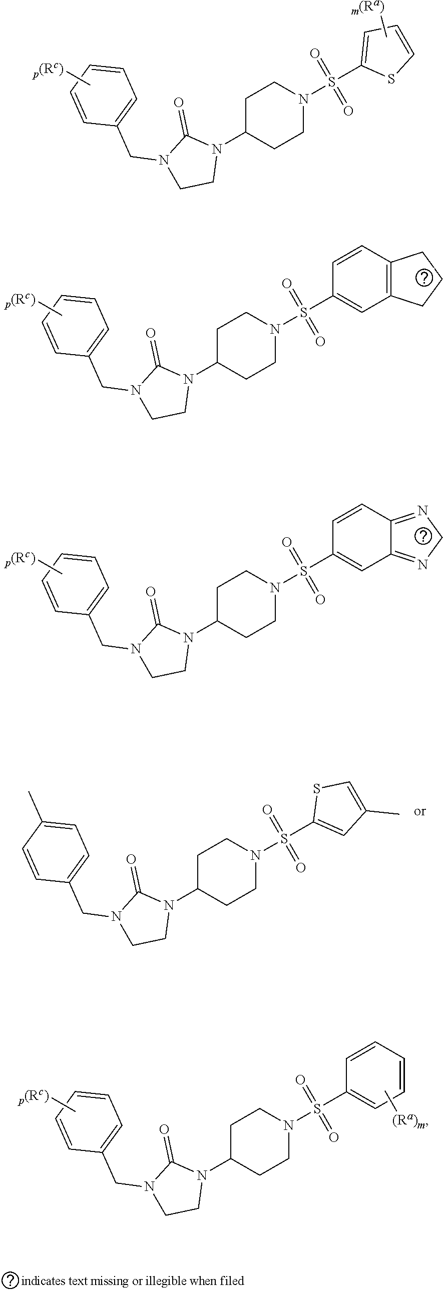
wherein P₁, P₂, and P₃, for each occurrence, are each independently selected from C and N, P₆ is independently selected from S and O.

2. The compound, tautomer, solvate, stereoisomer, or pharmaceutically acceptable salt of claim 1, wherein Ring A is a phenyl, pyridinyl, pyrimidinyl, pyrazinyl, thiazolyl, pyrazolyl, imidazolyl, pyrrolyl, pyridazinyl, piperazinyl, oxazolyl, isoxazolyl, triazolyl, cyclopentyl, cyclohexanyl, tetrahydro-furanyl, or tetrahydro-pyranyl group.

3. The compound, tautomer, solvate, stereoisomer, or pharmaceutically acceptable salt of claim 1, wherein Ring B is a phenyl, pyridinyl, thiazolyl, cyclopentenyl, cyclobutanyl, cyclohexanyl, piperidyl, or pyrrolidinyl group, or a 5- to 8-membered bicyclic group optionally containing one or two N atoms.

4. The compound, tautomer, solvate, stereoisomer, or pharmaceutically acceptable salt of claim 1, wherein Ring C is phenyl, pyridinyl, thiazolyl, isothiazolyl, oxazolyl, cyclopentyl, cyclopentenyl, cyclohexanyl, cyclohexenyl, isoxazolyl, tetrahydro-pyranyl, or dihydro-pyranyl group.

5. The compound of claim 1, wherein the compound has the following structural Formula IIa: 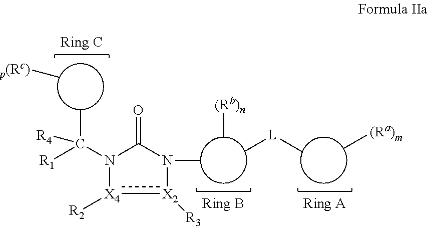
a tautomer thereof, a solvate or stereoisomer of the compound or the tautomer, or a pharmaceutically acceptable salt of the foregoing, wherein R¹ and R² do not join to form a 5- to 6-membered carbocycle or heterocycle.

6. The compound of claim 1, wherein the compound has the following structural Formula IIb: 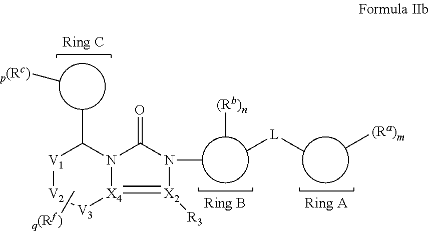
a tautomer thereof, a solvate or stereoisomer of the compound or the tautomer, or a pharmaceutically acceptable salt of the foregoing, wherein V₁, V₂, and V₃ are each independently selected from C, O, and N, R^{f}, for each occurrence, is independently selected from C₁-C₃ alkyl and halogen, and q is selected from 0, 1, and 2.

7. The compound of claim 1, wherein the compound has the following structural Formula IIc: 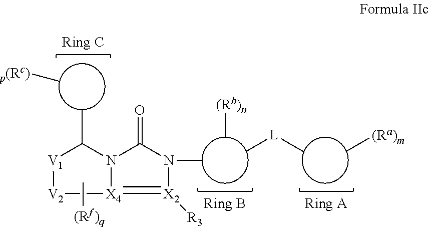
a tautomer thereof, a solvate or stereoisomer of the compound or the tautomer, or a pharmaceutically acceptable salt of the foregoing, wherein V₁ and V₂ are each independently selected from C, O, and N, R^{f}, for each occurrence, is independently selected from C₁-C₃ alkyl and halogen, and q is selected from 0, 1, and 2.

8. The compound of claim 1, wherein the compound has the following structural Formula IId: 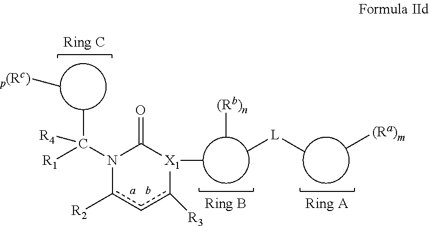
a tautomer thereof, a solvate or stereoisomer of the compound or the tautomer, or a pharmaceutically acceptable salt of the foregoing, wherein R¹ and R² do not join to form a 5- to 6-membered carbocycle or heterocycle.

9. The compound of claim 1, wherein the compound has the following structural Formula IIIa: 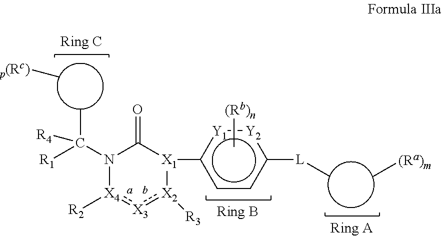
a tautomer thereof, a solvate or stereoisomer of the compound or the tautomer, or a pharmaceutically acceptable salt of the foregoing, wherein Y₁ and Y₂ are each independently selected from C and N.

10. The compound of claim 1, wherein the compound has the following structural Formula IIIb: 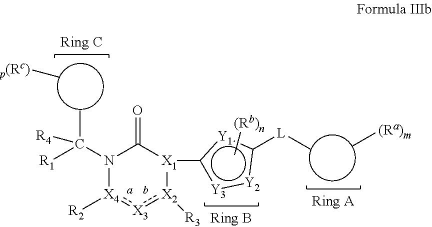
a tautomer thereof, a solvate or stereoisomer of the compound or the tautomer, or a pharmaceutically acceptable salt of the foregoing, wherein Y₁, is selected from S, C, O, and N, Y₂ and Y₃ are each independently selected from S, C, O and N.

11. The compound of claim 1, wherein the compound has the following structural Formula IIIc: 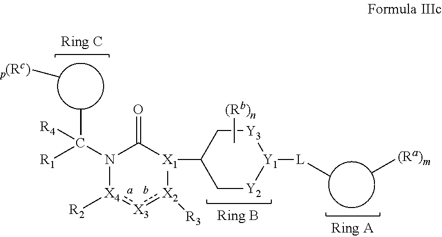
a tautomer thereof, a solvate or stereoisomer of the compound or the tautomer, or a pharmaceutically acceptable salt of the foregoing, wherein Y₁ is selected from C and N, Y₂ and Y₃ are each independently C or absent.

12. The compound of claim 1, wherein the compound has the following structural Formula IIId: 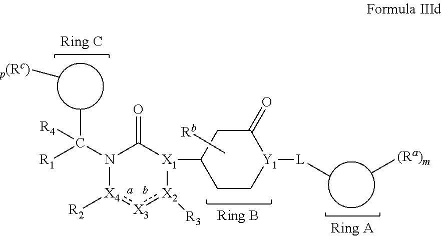
a tautomer thereof, a solvate or stereoisomer of the compound or the tautomer, or a pharmaceutically acceptable salt of the foregoing, wherein Y₁ is selected from C and N.

13. The compound of claim 1, wherein the compound has the following structural Formula IIIe: 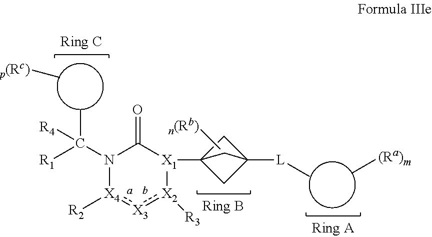
a tautomer thereof, a solvate or stereoisomer of the compound or the tautomer, or a pharmaceutically acceptable salt of the foregoing.

14. The compound of claim 1, wherein the compound has the following structural Formula IIIf: 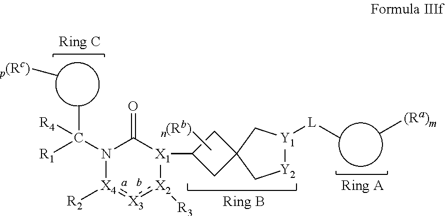
a tautomer thereof, a solvate or stereoisomer of the compound or the tautomer, or a pharmaceutically acceptable salt of the foregoing, wherein Y₁ is selected from C and N, and Y₂ is C or absent.

15. The compound of claim 1, wherein the compound has the following structural Formula IVa: 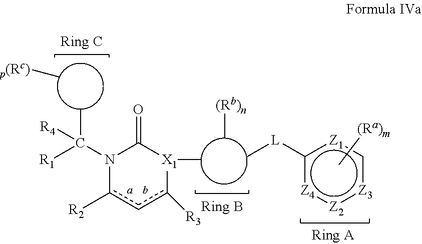
a tautomer thereof, a solvate or stereoisomer of the compound or the tautomer, or a pharmaceutically acceptable salt of the foregoing, wherein Z₁, Z₂, Z₃ and Z₄ are each independently selected from C and N.

16. The compound of any one of claim 1, wherein the compound has the following structural Formula IVb: 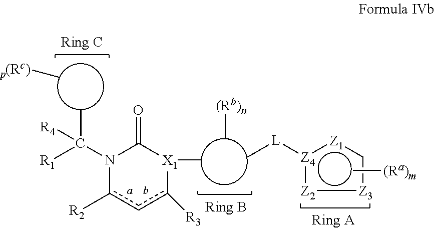
a tautomer thereof, a solvate or stereoisomer of the compound or the tautomer, or a pharmaceutically acceptable salt of the foregoing, wherein Z₁, Z₂, and Z₃, are each independently selected from S, O, C, and N, and Z₄ is selected from C and N.

17. The compound of claim 1, wherein the compound has the following structural Formula Va: 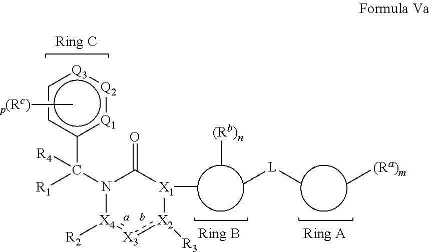
a tautomer thereof, a solvate or stereoisomer of the compound or the tautomer, or a pharmaceutically acceptable salt of the foregoing, wherein Q₁, Q₂, and Q₃ are each independently selected from C and N.

18. The compound of claim 1, wherein the compound has the following structural Formula Vb: 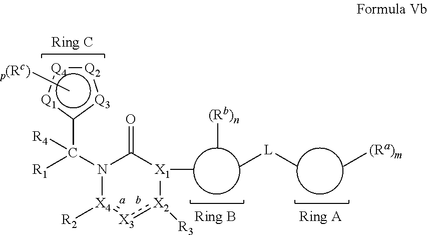
a tautomer thereof, a solvate or stereoisomer of the compound or the tautomer, or a pharmaceutically acceptable salt of the foregoing, wherein Q₁, Q₂, Q₃, and Q₄ are each independently selected from C, N, S, and O.

19. The compound of claim 1, wherein the compound has the following structural Formula Vc: 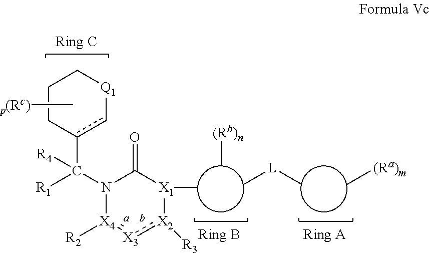
a tautomer thereof, a solvate or stereoisomer of the compound or the tautomer, or a pharmaceutically acceptable salt of the foregoing, wherein Q₁ is C, O, or absent.

20. The compound of claim 1, wherein the compound has the following structural Formula Vd: 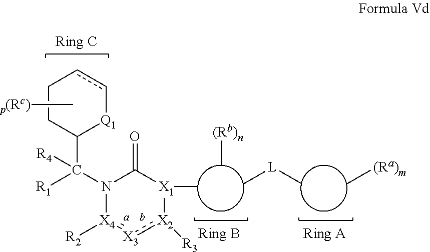
a tautomer thereof, a solvate or stereoisomer of the compound or the tautomer, or a pharmaceutically acceptable salt of the foregoing, wherein Q₁ is C or absent.

21. The compound of claim 1, wherein the compound has the following structural Formula VIa: 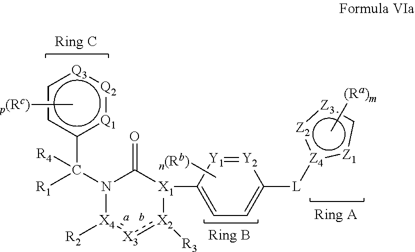
a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z₁, Z₂, and Z₃, are each independently selected from S, O, C, and N, Z₄ is selected from C and N.

22. The compound of claim 1, wherein the compound has the following structural Formula VIb: 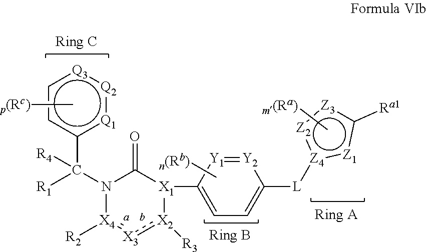
a tautomer thereof, a solvate or stereoisomer of the compound or the tautomer, or a pharmaceutically acceptable salt of the foregoing, wherein R^{a}, for each occurrence, is independently selected from absent, halogen, CN, NO₂, NH₂, —NH(C₁-C₃ alkyl), —OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{a1} is selected from optionally substituted C₁-C₃ alkyl, optionally substituted 3- to 6-membered cycloalkyl, optionally substituted 3- to 6-membered heterocyclyl, —NC(═O)R^{p2}, and —NR^{p2}R^{q2} wherein R^{p2} and R^{q2}, for each occurrence, are each independently selected from hydrogen and optionally substituted C₁-C₆ alkyl, or R^{p2} and R^{q2} join and form an optionally substituted 3 to 10-membered heterocyclyl.

23. The compound of claim 1, a tautomer thereof, a solvate or stereoisomer of the compound or the tautomer, or a pharmaceutically acceptable salt of the foregoing, wherein R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, and NO₂, and R^{a1} is selected from COOMe, COOEt, 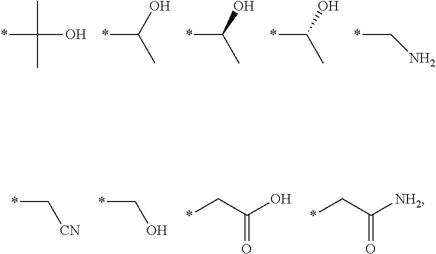
—NR^{p2}R^{q2}, wherein R^{p2} and R^{q2} join and form a 3 to 10-membered heterocyclyl optionally substituted by 1 to 2 groups selected from halogen, OH, CN, —O(C₁-C₃ alkyl), NH₂, NHBoc, NH(C₁-C₃ alkyl), and C₁-C₃ alkyl optionally substituted with 1 to 3 groups selected from halogen, CN, NH₂, NHBoc, and OH.

24. The compound of claim 1, wherein the compound has the following structural Formula VIc: 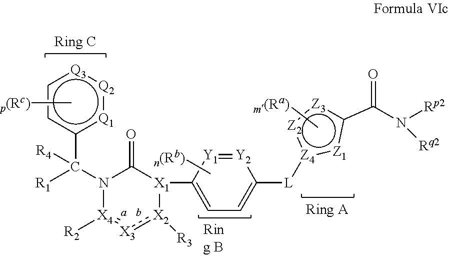
a tautomer thereof, a solvate or stereoisomer of the compound or the tautomer, or a pharmaceutically acceptable salt of the foregoing, wherein R^{a}, for each occurrence, is independently selected from absent, halogen, CN, NO₂, NH₂, —NH(C₁-C₃ alkyl), OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{p2} and R^{q2} are independently selected from H, optionally substituted C₁ to C₆ alkyl, optionally substituted 3 to 10-membered cycloalkyl, optionally substituted 3 to 10-membered heterocyclyl, and optionally substituted 3 to 10-membered heteroaryl.

25. The compound of claim 1, a tautomer thereof, a solvate or stereoisomer of the compound or the tautomer, or a pharmaceutically acceptable salt of the foregoing, wherein R^{a}, for each occurrence, is independently selected from absent, CH₃, CF₂, F, Cl, CN, NH₂, NHCH₃, and OH.

26. The compound of claim 1, wherein the compound has the following structural Formula VId: 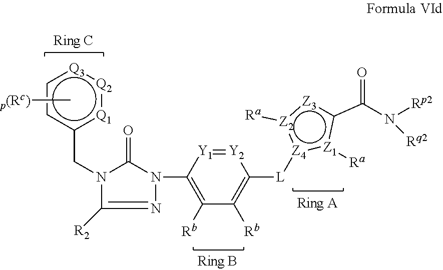
a tautomer thereof, a solvate or stereoisomer of the compound or the tautomer, or a pharmaceutically acceptable salt of the foregoing, wherein L is selected from —O—, —N(R^{x})—, —CH₂—, —S—, —S(═O)—, —S(═O)₂—, and C₁-C₃ alkylene optionally substituted by 1 to 2 groups selected from C₁-C₂ alkyl, wherein the C₁-C₂ alkyl of the C₁-C₃ alkylene of L optionally join to form a C₃-C₄ cycloalkyl, R^{a}, for each occurrence, is independently selected from H and C₁-C₃ alkyl, R^{b}, for each occurrence, is independently selected from absent, F, Cl, Br, CH₃, and CN, R^{c} is selected from F, Cl, Br, CH₃, —OCH₃, and CN, p is 0, 1, or 2, provided that when L is —O— or —N(R^{x})—, Z₄ is C, and when L is —CH₂—, —S—, —S(═O)—, —S(═O)₂—, or C₁-C₃ alkylene, Z₄ is C or N.

27. The compound of claim 1, wherein the compound has the following structural Formula VIe: 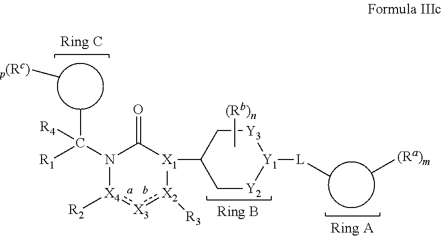
a tautomer thereof, a solvate or stereoisomer of the compound or the tautomer, or a pharmaceutically acceptable salt of the foregoing, wherein V₁ and V₂ are each independently selected from C, N, and O, L is selected from —O—, —N(R^{x})—, —CH₂—, —S—, —S(═O)—, —S(═O)₂—, and C₁-C₃ alkylene optionally substituted by 1 to 2 groups selected from C₁-C₂ alkyl, wherein the C₁-C₂ alkyl of the C₁-C₃ alkylene of L optionally join to form a C₃-C₄ cycloalkyl, R^{a}, for each occurrence, is independently selected from H and C₁-C₃ alkyl, R^{b}, for each occurrence, is independently selected from absent, F, Cl, Br, CH₃, and CN, R^{c} is selected from F, Cl, Br, —OCH₃, CH₃, and CN, R^{f}, for each occurrence, is independently selected from C₁-C₃ alkyl and halogen, p is 0, 1, or 2, q is 0, 1, or 2, provided that when L is —O— or —N(R^{x})—, Z₄ is C, and when L is —CH₂—, —S—, —S(═O)—, —S(═O)₂—, or C₁-C₃ alkylene, Z₄ is C or N.

28. The compound of claim 1, wherein the compound has the following structural Formula VIf: 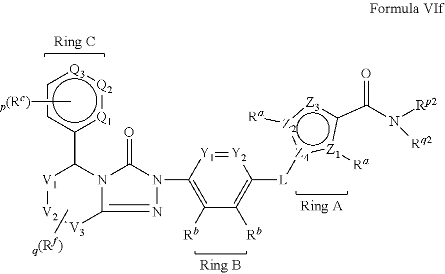
a tautomer thereof, a solvate or stereoisomer of the compound or the tautomer, or a pharmaceutically acceptable salt of the foregoing, wherein V₁, V₂, and V₃ are each independently selected from C, N, and O, L is selected from —O—, —N(R^{x})—, —CH₂—, —S—, —S(═O)—, —S(═O)₂—, and C₁-C₃ alkylene optionally substituted by 1 to 2 groups selected from C₁-C₂ alkyl, wherein the C₁-C₂ alkyl of the C₁-C₃ alkylene of L optionally join to form a C₃-C₄ cycloalkyl, R^{a}, for each occurrence, is independently selected from H and C₁-C₃ alkyl, R^{b}, for each occurrence, is independently selected from absent, F, Cl, Br, CH₃, and CN, R^{c} is selected from F, Cl, Br, CH₃, —OCH₃, and CN, R^{f}, for each occurrence, is independently selected from C₁-C₃ alkyl and halogen, p is 0, 1, or 2, q is 0, 1, or 2, provided that when L is —O— or —N(R^{x})—, Z₄ is C, and when L is —CH₂—, —S(═O)—, —S(═O)₂—, or C₁-C₃ alkylene optionally substituted by C₃-C₄ cycloalkyl, Z₄ is C or N.

29. The compound of claim 1, wherein the compound has the following structural Formula VIIa: 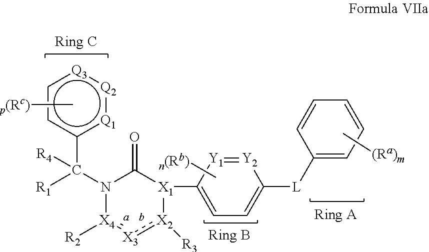
a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N.

30. The compound of claim 1, wherein the compound has the following structural Formula VIIb: 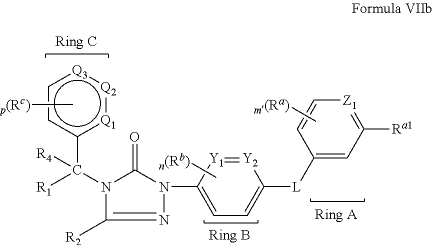
a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z₁ is selected from C and N, R^{a}, for each occurrence, is independently selected from absent, halogen, CN, NO₂, NH₂, —NH(C₁-C₃ alkyl), —OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{a1} is selected from optionally substituted C₁-C₃ alkyl, optionally substituted 3- to 6-membered cycloalkyl, optionally substituted 3- to 6-membered heterocyclyl, —NC(═O)R^{p2}, and —NR^{p2}R^{q2}, wherein R^{p2} and R^{q2}, for each occurrence, are each independently selected from hydrogen and optionally substituted C₁-C₆ alkyl, or R^{p2} and R^{q2} join and form an optionally substituted 3 to 10-membered heterocyclyl.

31. The compound of claim 1, a tautomer thereof, a solvate or stereoisomer of the compound or the tautomer, or a pharmaceutically acceptable salt of the foregoing, wherein R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, and NO₂, and R^{a1} is selected from COOMe, COOEt, 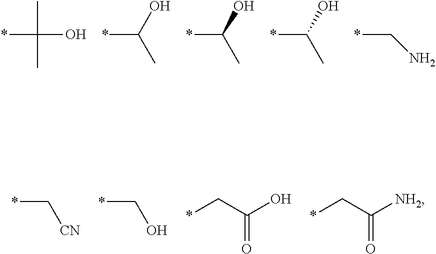
—NR^{p2}R^{q2}, wherein R^{p2} and R^{q2} join and form a 3 to 10-membered heterocyclyl optionally substituted by 1 to 2 groups selected from halogen, OH, CN, —O(C₁-C₃ alkyl), NH₂, NHBoc, NH(C₁-C₃ alkyl), and C₁-C₃ alkyl optionally substituted with 1 to 3 groups selected from halogen, CN, NH₂, NHBoc, and OH.

32. The compound of claim 1, wherein the compound has the following structural Formula VIIc: 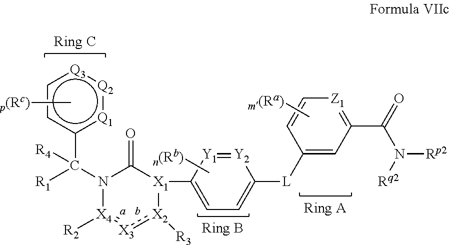
a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z₁ is selected from C and N, R^{a}, for each occurrence, is independently selected from absent, halogen, CN, NO₂, NH₂, —NH(C₁-C₃ alkyl), —OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, m′ is 0, 1, and 2, R^{P2} and R^{q2}, are independently selected from H, optionally substituted C₁ to C₆ alkyl, optionally substituted 3 to 10-membered cycloalkyl, optionally substituted 3 to 10-membered heterocyclyl, and optionally substituted 3 to 10-membered heteroaryl.

33. The compound of claim 1, a tautomer thereof, a solvate or stereoisomer of the compound or the tautomer, or a pharmaceutically acceptable salt of the foregoing, wherein R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, and NO₂, and wherein the 3 to 10-membered cycloalkyl, 3 to 10-membered heterocyclyl, and 3 to 10-membered heteroaryl of R^{P2} and R^{q2} are optionally substituted with 1 to 2 groups selected from halogen, OH, CN, —O(C₁-C₃ alkyl), NH₂, NHBoc, NH(C₁-C₃ alkyl), and C₁-C₃ alkyl optionally substituted with halogen, CN, NH₂, NHBoc, and OH.

34. The compound of claim 1, wherein the compound has the following structural Formula VIIIa, VIIIb, or VIIIc: 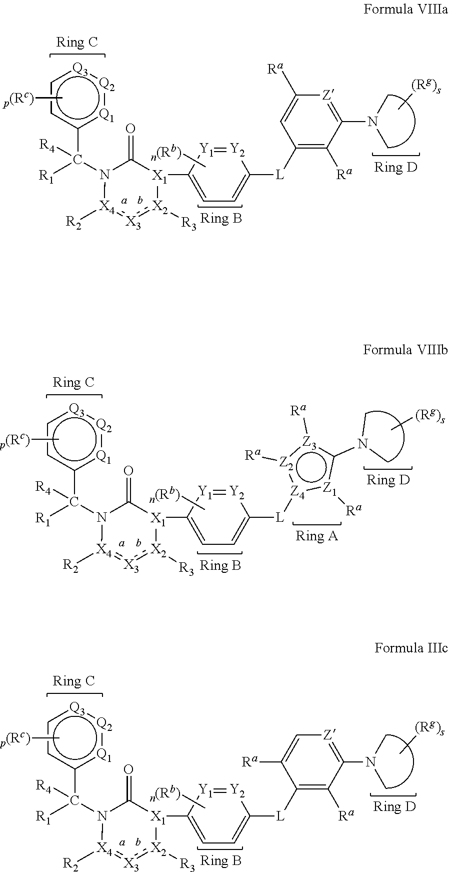
a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z′ is selected from C and N, Z₁, Z₂, and Z₃, are each independently selected from S, O, C, and N, Z₄ is selected from C and N, R^{a}, for each occurrence, is independently selected from absent, halogen, CN, NO₂, NH₂, —NH(C₁-C₃ alkyl), —OH, —O(C₁-C₃ alkyl), —C(═O)H, —C(═O)O(C₁-C₃alkyl), —C(═O)NH₂, and C₁-C₃ alkyl optionally substituted by 1 to 3 groups selected from halogen, OH, CN, and NH₂, Ring D is 3 to 10-membered heterocyclyl, R^{g}, for each occurrence, is independently selected from halogen, OH, CN, —O(C₁-C₃ alkyl), NH₂, NHBoc, NH(C₁-C₃ alkyl), and C₁-C₃ alkyl optionally substituted with 1 to 3 groups selected from halogen, CN, NH₂, NHBoc, and OH, s is an integer selected from 0, 1, and 2.

35. The compound of claim 1, wherein the compound has the following structural Formula VIIId, VIIIe, or VIIIf: 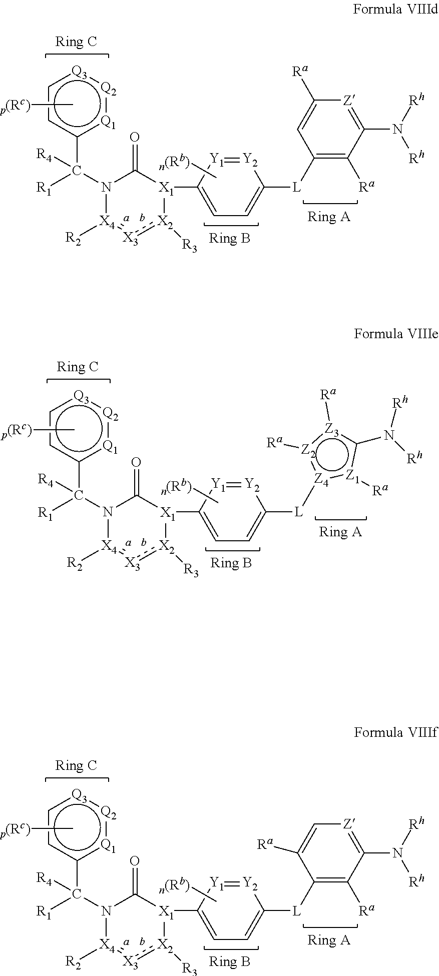
a tautomer thereof, a solvate or stereoisomer of the compound or the tautomer, or a pharmaceutically acceptable salt of the foregoing, wherein Q₁, Q₂, and Q₃ are each independently selected from C and N, Y₁ and Y₂ are each independently selected from C and N, Z′ is selected from C and N, Z₁, Z₂ and Z₃ are each independently selected from S, O, C and N, Z₄ is selected from C and N, R^{a}, for each occurrence, is independently selected from absent, halogen, CN, NO₂, NH₂, NH(C₁-C₃ alkyl), OH, —O(C₁-C₃ alkyl), —C(═O)H, —C(═O)O(C₁-C₃ alkyl), —C(═O)NH₂, and C₁-C₃ alkyl optionally substituted by 1 to 3 groups selected from halogen, OH, CN, and NH₂, R^{h}, for each occurrence, is independently selected from H, C₁-C₃ alkyl optionally substituted with 1-3 groups selected from halogen and 3 to 4-membered cycloalkyl, 3-6 membered cycloalkyl optionally substituted by 1 to 3 groups selected from halogen and C₁-C₃ alkyl, and 3-6 membered heterocyclyl optionally substituted by 1 to 3 groups selected from halogen and C₁-C₃ alkyl.

36. The compound, tautomer, solvate, stereoisomer, or pharmaceutically acceptable salt of claim 1, wherein: 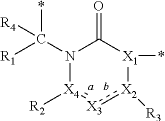
of Formula I is selected from: 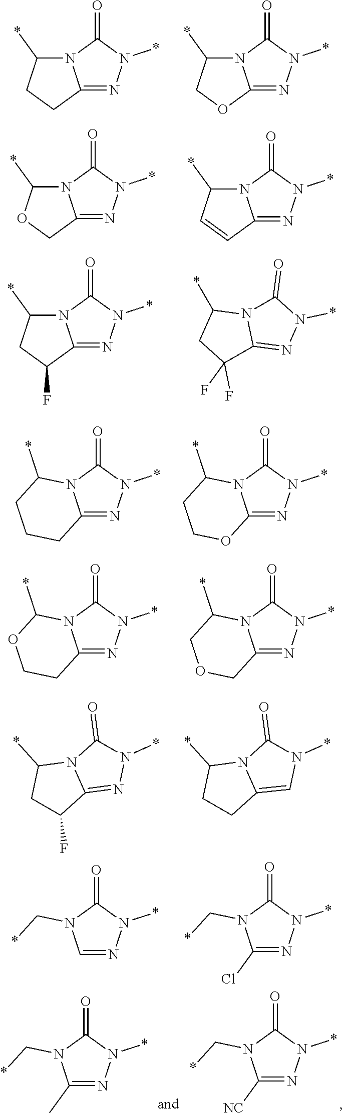
Ring B substituted with n groups of R^{b} is selected from: 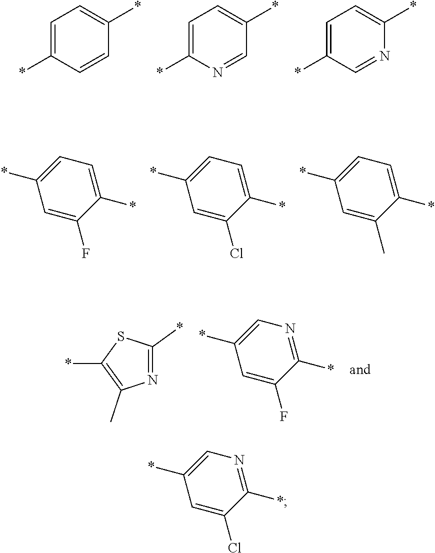
Ring C substituted with p groups of R^{c} is selected from: 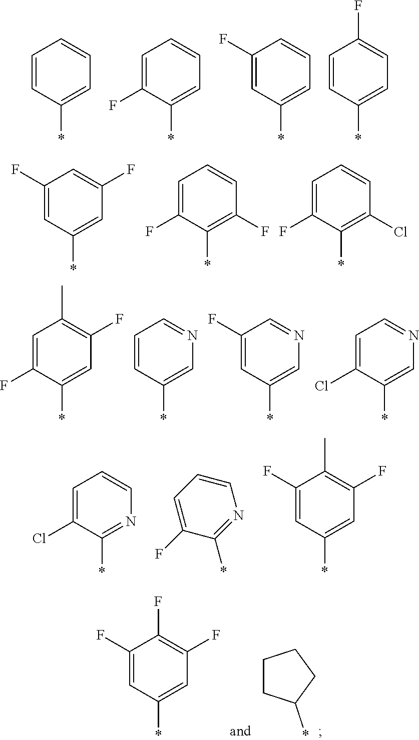
and L is selected from:

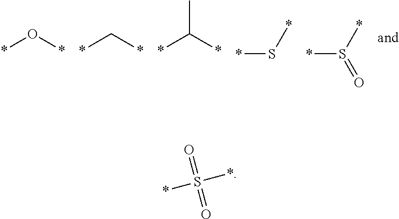

37. The compound, tautomer, solvate, stereoisomer, or pharmaceutically acceptable salt of claim 1, wherein Ring A substituted with m groups of R^{a} is selected from: 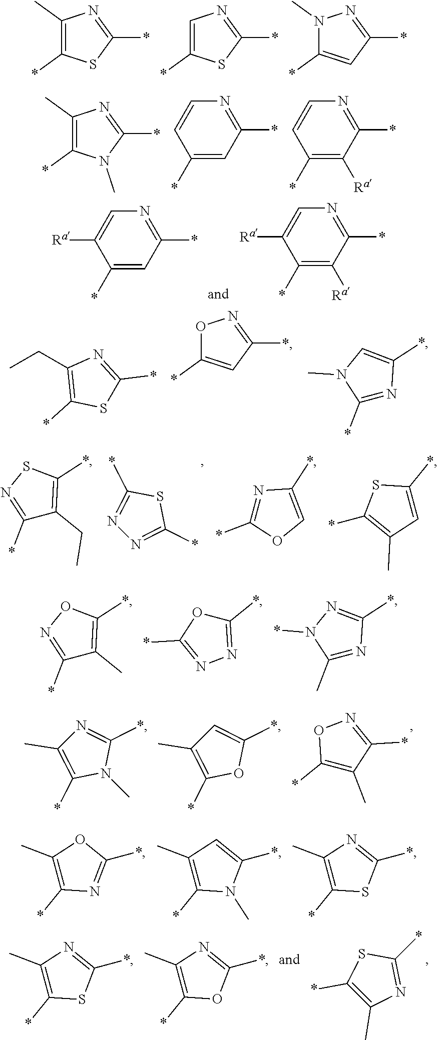
wherein R^{a′} for each occurrence, is independently selected from F, Cl, —OCH₃, CH₃, NH₂, and CN; L is —O—; the position denoted by the * on the left side of the above structures is connected to L, and the position denoted by the * on the right side of the above structures is connected to an R^{a}.

38. The compound, tautomer, solvate, stereoisomer, or pharmaceutically acceptable salt of claim 1, wherein Ring A substituted by m groups of R^{a} is selected from: 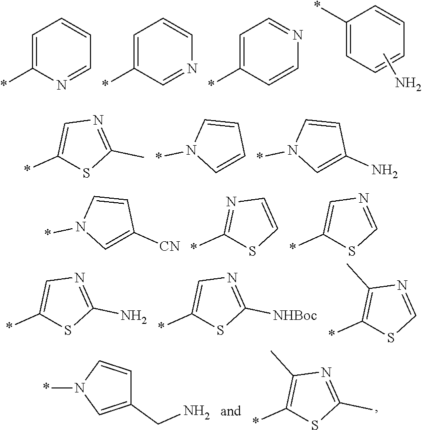
and L is —SO₂—.

39. The compound, tautomer, solvate, stereoisomer, or pharmaceutically acceptable salt of claim 1, wherein 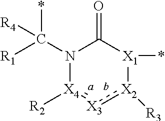
of Formula I is selected from: 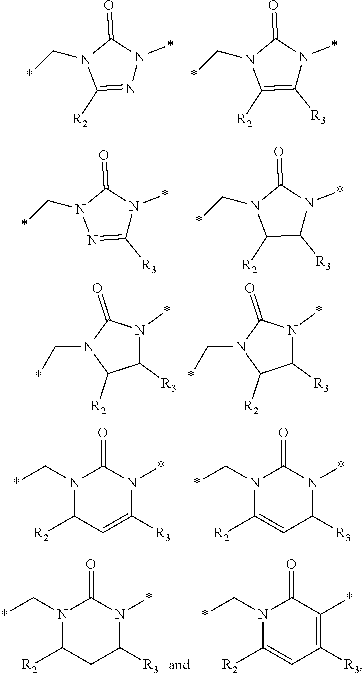
wherein, R² is selected from H, halogen, CN, —NH₂, OH, OCH₃, ═O, and C₁ to C₃ alkyl optionally substituted by 1 to 3 groups selected from halogen.

40. The compound, tautomer, solvate, stereoisomer, or pharmaceutically acceptable salt of claim 1, wherein 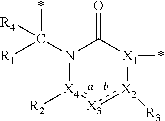
of Formula I is selected from:

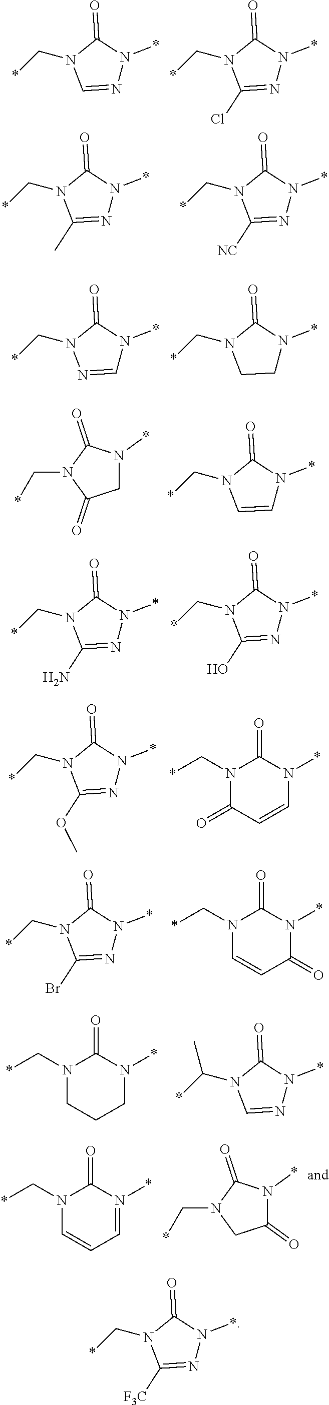

41. The compound, tautomer, solvate, stereoisomer, or pharmaceutically acceptable salt of claim 1, wherein 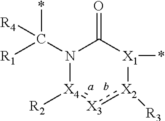
of Formula I is selected from: 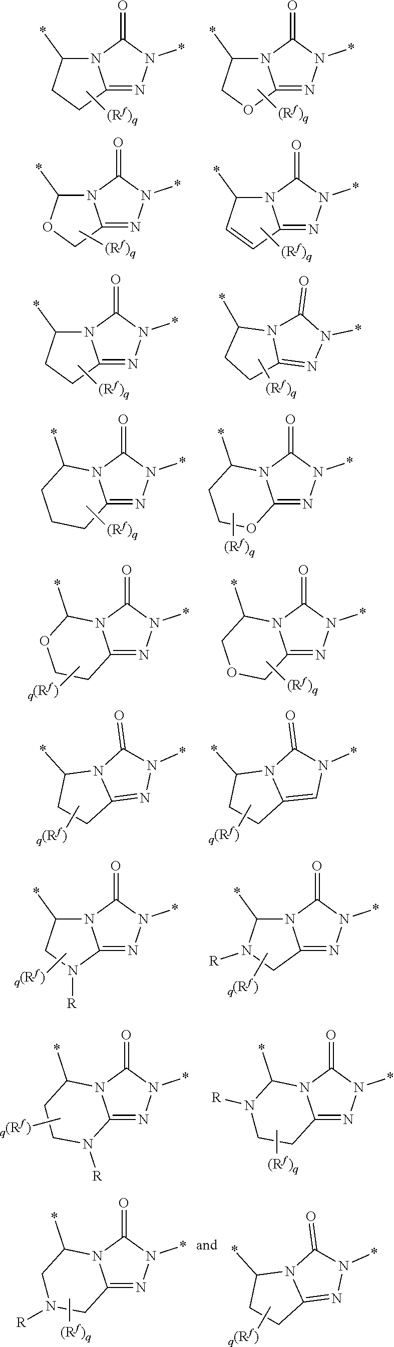
wherein R^{f} for each occurrence, is independently selected from C₁-C₂ alkyl and halogen, and q is selected from 0, 1, and 2.

42. The compound, tautomer, solvate, stereoisomer, or pharmaceutically acceptable salt of claim 1, wherein the 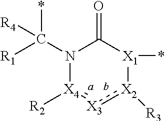
of Formula I is selected from:

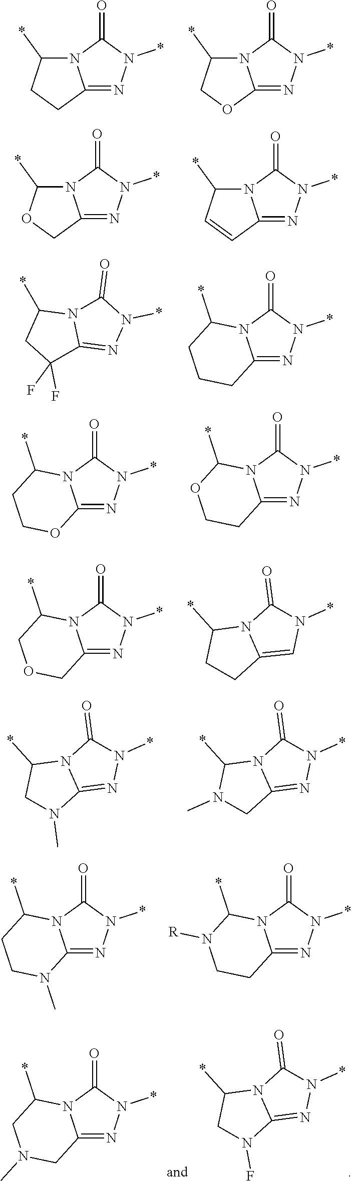

43. The compound, tautomer, solvate, stereoisomer, or pharmaceutically acceptable salt of claim 1, wherein Ring A is selected from: 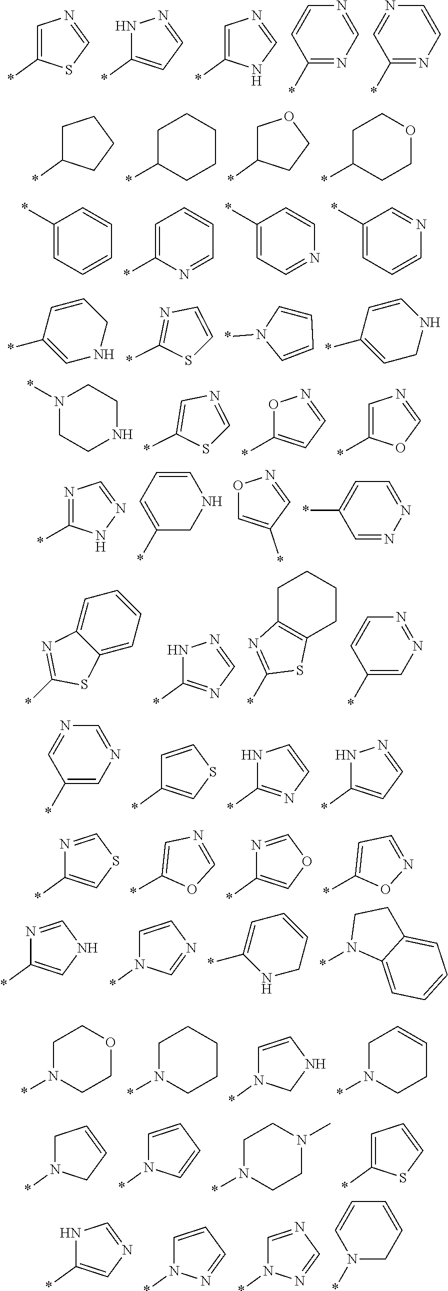 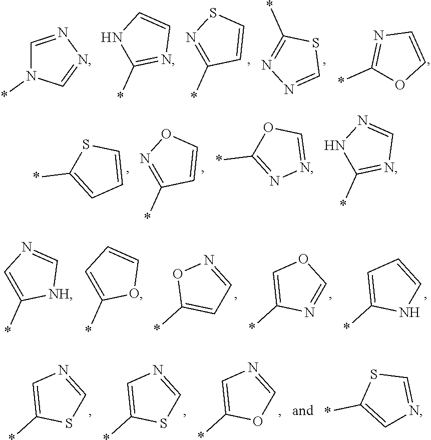
wherein Ring A is substituted with m groups of R^{a}.

44. The compound, tautomer, solvate, stereoisomer, or pharmaceutically acceptable salt of claim 1, wherein Ring A substituted by m groups of R^{a} is selected from: 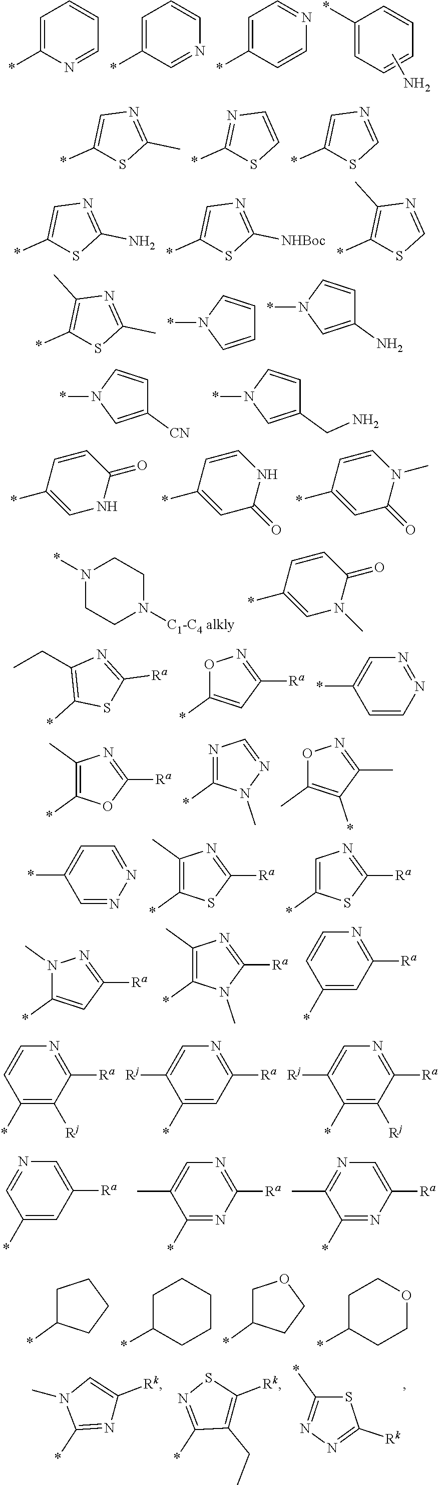 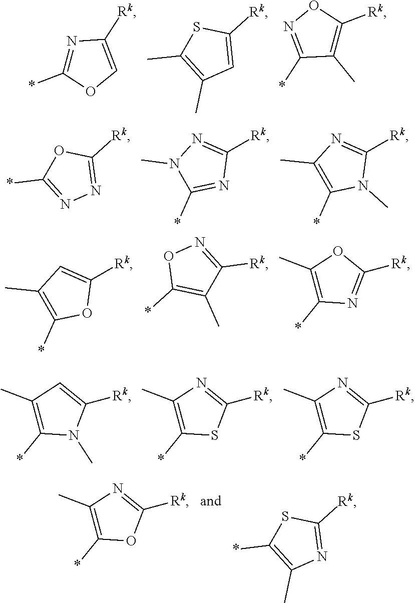
wherein R^{k} is selected from —C(═O)NH₂, —C(═O)NHCH₃, —C(═O)N(CH₃)₂, and R₁, for each occurrence, is independently selected from F, Cl, CH₃, and CN.

45. The compound, tautomer, solvate, stereoisomer, or pharmaceutically acceptable salt of claim 1, wherein Ring B is selected from: 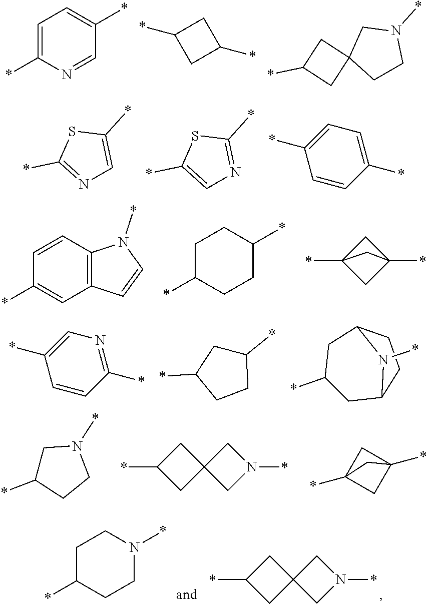
wherein Ring B is substituted with n groups of R^{b}.

46. The compound, tautomer, solvate, stereoisomer, or pharmaceutically acceptable salt of claim 1, wherein Ring B substituted by n groups of R^{b} is selected from:

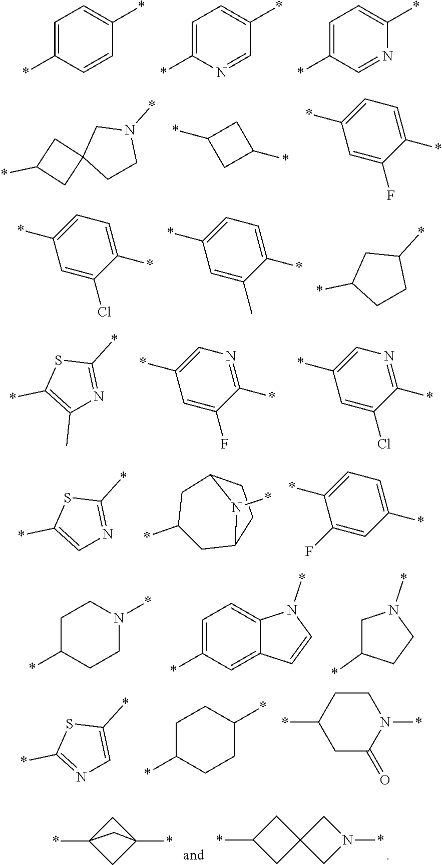

47. The compound, tautomer, solvate, stereoisomer, or pharmaceutically acceptable salt of claim 1, wherein Ring C is selected from: 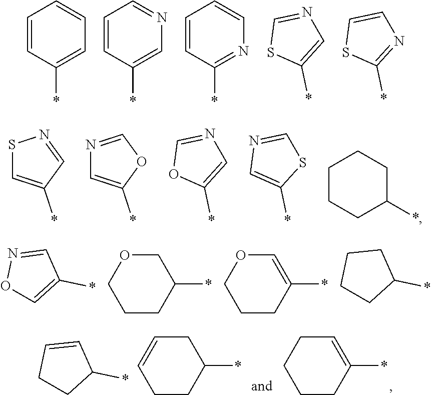
wherein Ring C is substituted with p groups of R^{c}.

48. The compound, tautomer, solvate, stereoisomer, or pharmaceutically acceptable salt of claim 1, wherein Ring C substituted by p groups of R^{c} is selected from:

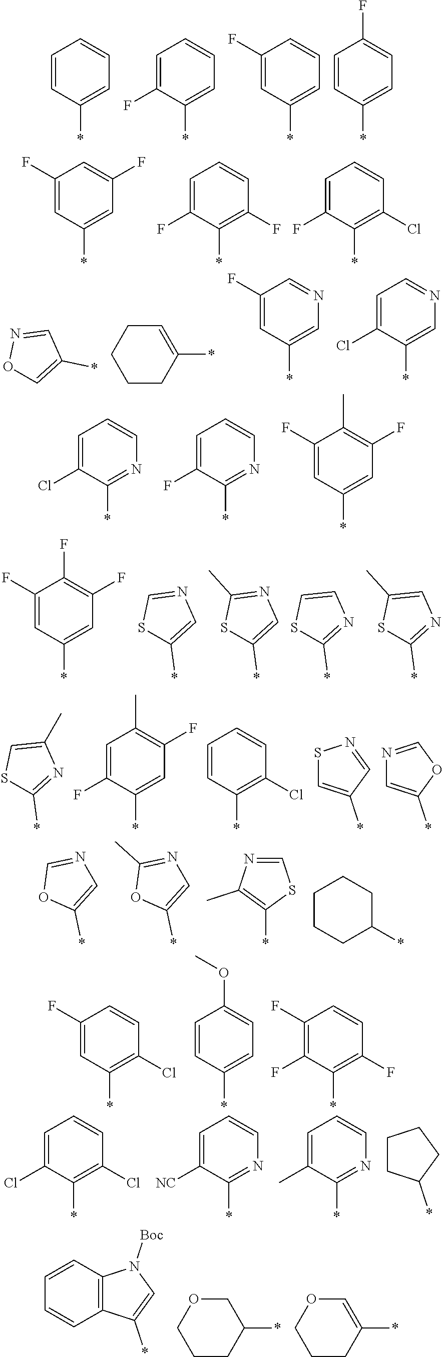

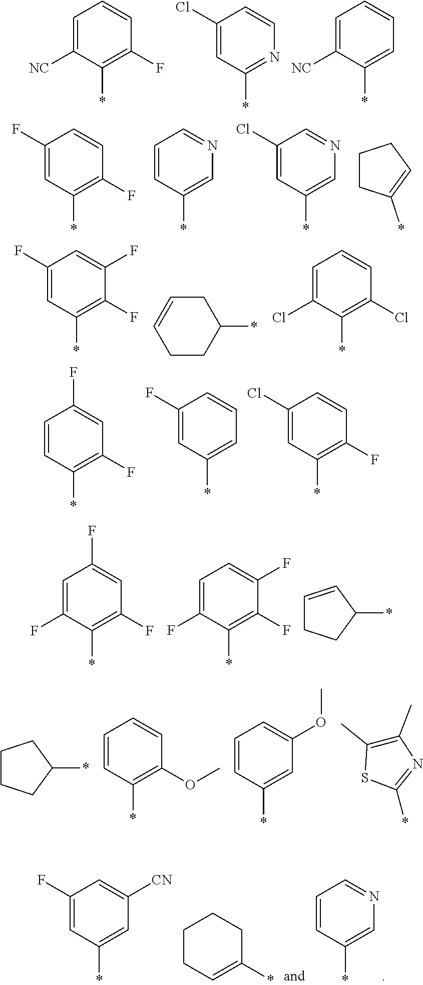

49. The compound, tautomer, solvate, stereoisomer, or pharmaceutically acceptable salt of claim 1, wherein R^{a}, for each occurrence, is independently selected from absent; halogen; cyano; ═O; NO₂;
C₁ to C₆ alkyl optionally substituted by 1 to 4 groups selected from halogen, CN, —OR^{s}, —C(═O)NR^{p}R^{q}, —C(═O)OR^{s}, —N3, ═NRp, ═NOR^{s}, —NR^{p}R^{q}, 3 to 10-membered cycloalkyl, and 3 to 10-membered heterocyclyl, wherein the 3 to 10-membered cycloalkyl and the 3 to 10-membered heterocyclyl are each optionally substituted by 1 to 4 groups selected from C₁ to C₆ alkyl, halogen, and OR^{s};
C₂ to C₆ alkenyl optionally substituted by 1 to 4 groups selected from halogen, CN, —OR^{s}, —C(═O)NR^{p}R^{q}, —C(═O)OR^{s}, —N3, ═NRp, ═NOR^{s}, —NR^{p}R^{q}, 3 to 10-membered cycloalkyl, and 3 to 10-membered heterocyclyl, wherein the 3 to 10-membered cycloalkyl and the 3 to 10-membered heterocyclyl are each optionally substituted by 1 to 4 groups selected from C₁ to C₆ alkyl, halogen, and OR^{s};
C₂ to C₆ alkynyl optionally substituted by 1 to 4 groups selected from halogen, CN, —OR^{s}, —C(═O)NR^{p}R^{q}, —C(═O)OR^{s}, —N3, ═NRp, =NOR^{s}, —NR^{p}R^{q}, 3 to 10-membered cycloalkyl, and 3 to 10-membered heterocyclyl, wherein the 3 to 10-membered cycloalkyl and the 3 to 10-membered heterocyclyl are each optionally substituted by 1 to 4 groups selected from C₁ to C₆ alkyl, halogen, and OR^{s};
3 to 10-membered cycloalkyl optionally substituted by 1 to 4 groups selected from halogen, CN, OR^{s}, —C(═O)NR^{p}R^{q}, —C(═O)OR^{s}, and —NR^{p}R^{q};
3 to 10-membered heterocyclyl optionally substituted by 1 to 4 groups selected from halogen, CN, OR^{s}, —C(═O)NR^{p}R^{q}, —C(═O)OR^{s}, and —NR^{p}R^{q};
—C(═O)R^{s};
—C(═O)OR^{s};
—C(═O)(C═O)OR^{s};
—C(═O)NR^{p}R^{q}NR^{p}R^{q};
—C(═O)NR^{p}R^{q}OR^{s};
—C(═O)NR^{p}R^{q};
—NR^{p}R_{q};
—NR^{p}C(═O)R^{s}, wherein R^{p} and R^{s} are defined below in this claim or the R^{p} and R^{s} of NR^{p}C(═O)R^{s} join and form a 5 to 10-membered heterocyclyl;
—NR^{p2}C(═O)OR^{s2}, wherein R^{p2} and R^{s2} are defined below in this claim or the R^{p2} and R^{s2} of —NR^{p2}C(═O)OR^{s2} join and form a 5 to 10-membered heterocyclyl;
—OR^{s}:
wherein:
R^{p} and R^{q}, for each occurrence, are independently selected from hydrogen and C₁-C₆ alkyl, or R^{p} and R^{q} join and form a 3 to 10-membered heterocyclyl, wherein:
the C₁-C₄ alkyl of any one of R^{p} and R^{q} is optionally substituted with 1 to 3 groups selected from halogen, —NR^{p1}C(═O)OR^{s1}, cyano, —OH, —OR^{s1}, —O(C₁ to C₃ alkyl)OR^{s1}, 3 to 10-membered cycloalkyl, 3 to 10-membered heterocyclyl, and phenyl; wherein
the 3 to 10-membered heterocyclyl of any one of R^{p} and R^{q}, and the 3 to 10-membered cycloalkyl and 3 to 10-membered heterocyclyl of the C₁-C₄ alkyl of any one of R^{p} and R^{q}, are each optionally substituted with 1 to 3 groups selected from halogen, CN, ═O, NR^{p1}R^{q1}, OR^{s1}, —NR^{p1}C(═O)R^{s1}, —NR^{p1}C(═O)OR^{s1}, 3 to 10-membered cycloalkyl, and C₁ to C₃ alkyl optionally substituted with C₃-C₄ cycloalkyl;
R^{s}, for each occurrence, is independently selected from hydrogen, C₁-C₆ alkyl, phenyl, 5 to 6-membered heteroaryl, 3 to 10-membered cycloalkyl, and 3 to 10-membered heterocyclyl,
wherein the C₁-C₆ alkyl, phenyl, 5 to 6-membered heteroaryl,
3 to 10-membered cycloalkyl, and 3 to 10-membered heterocyclyl of R^{s} are each optionally substituted with 1 to 3 groups selected from halogen, NR^{p1}R^{q1}, —NR^{p1}C(═O)OR^{s1}, cyano, —OH, —O(C₁ to C₃ alkyl), —O(C₁ to C₃ alkyl)OH, —O(C₁ to C₃ alkyl)O(C₁ to C₃ alkyl), 3 to 10-membered cycloalkyl, 3 to 10-membered heterocyclyl, and phenyl;
R^{p1} and R^{q1}, for each occurrence, are each independently selected from hydrogen and C₁-C₄ alkyl optionally substituted by 1 to 3 groups of halogen, CN, and OH;
R^{s1}, for each occurrence, is independently selected from hydrogen and C₁-C₄ alkyl optionally substituted by 1 to 3 groups of halogen, CN, and OH;
R^{p2}, for each occurrence, is independently selected from hydrogen and C₁-C₄ alkyl optionally substituted by 1 to 3 groups of halogen, CN, and OH;
R^{s2}, for each occurrence, is independently selected from hydrogen and C₁-C₄ alkyl optionally substituted by 1 to 3 groups of halogen, CN, and OH.

50. The compound, tautomer, solvate, stereoisomer, or pharmaceutically acceptable salt of claim 1, wherein R^{a} is selected from absent, NH₂, NO₂, ═O, cyano, I, F, Cl, Br, —CH₃, —CH(CH₃)₂, —CH₂CN, —CF₃, —CH₂OH, —CH₂CH₂OH, —C(CH₃)₂OH, —CH₂C(CH₃)₂OH, —CHF₂, —CHCH₃OH, —CH₂CONH₂, —CH₂COOH, —CHCH₃NH₂, —CH₂OH, —CH₂CH₂OH, —CH₂N₃, —CH₂NH₂, —CH₂OCH₃,

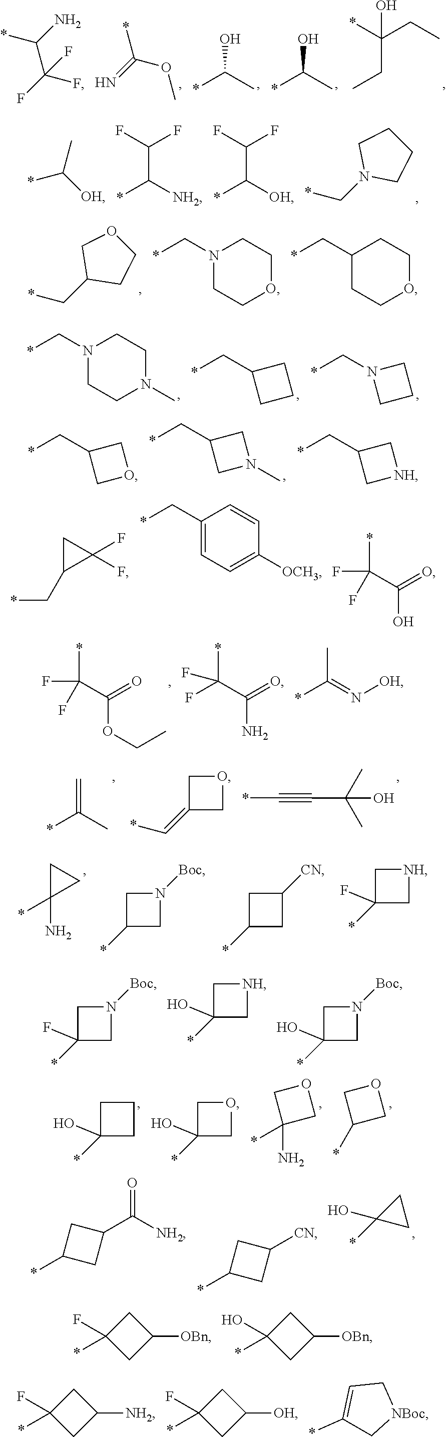

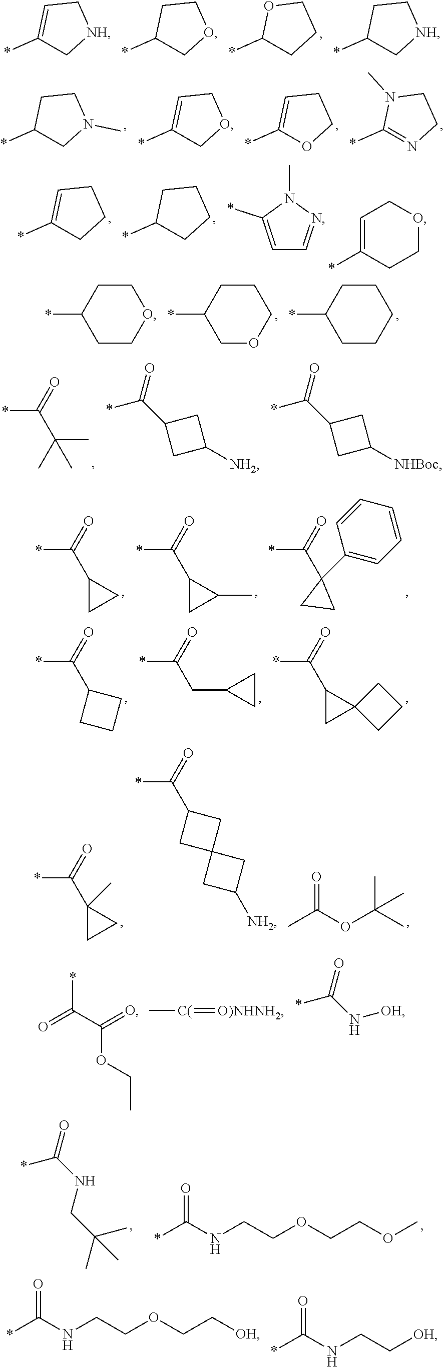

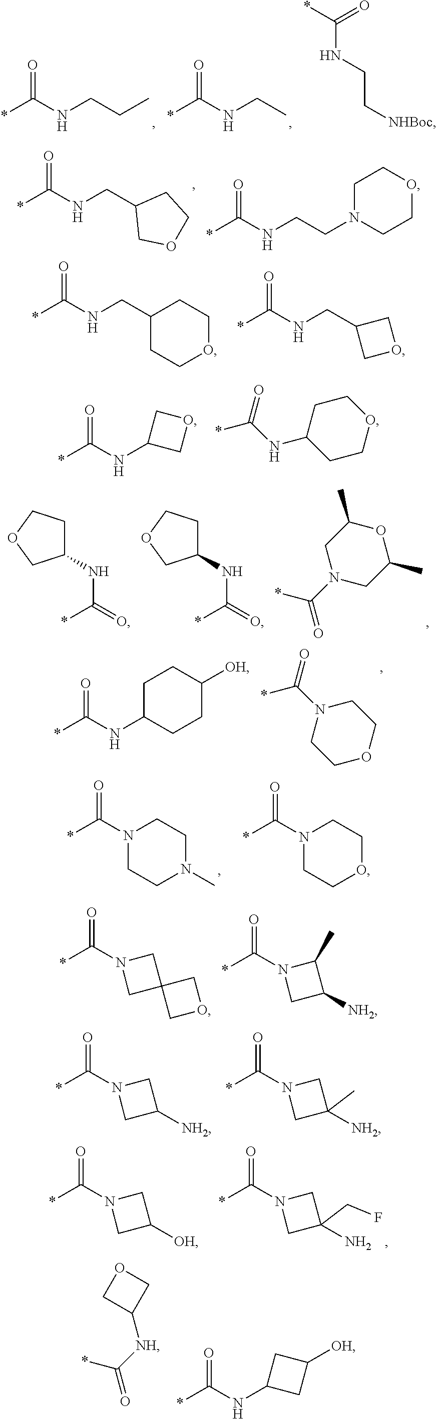

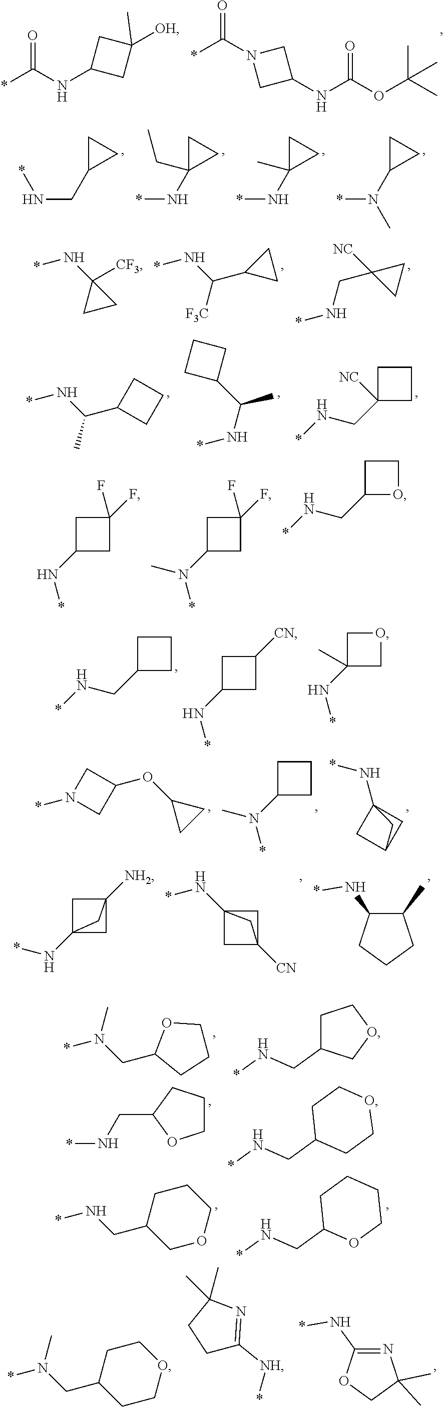

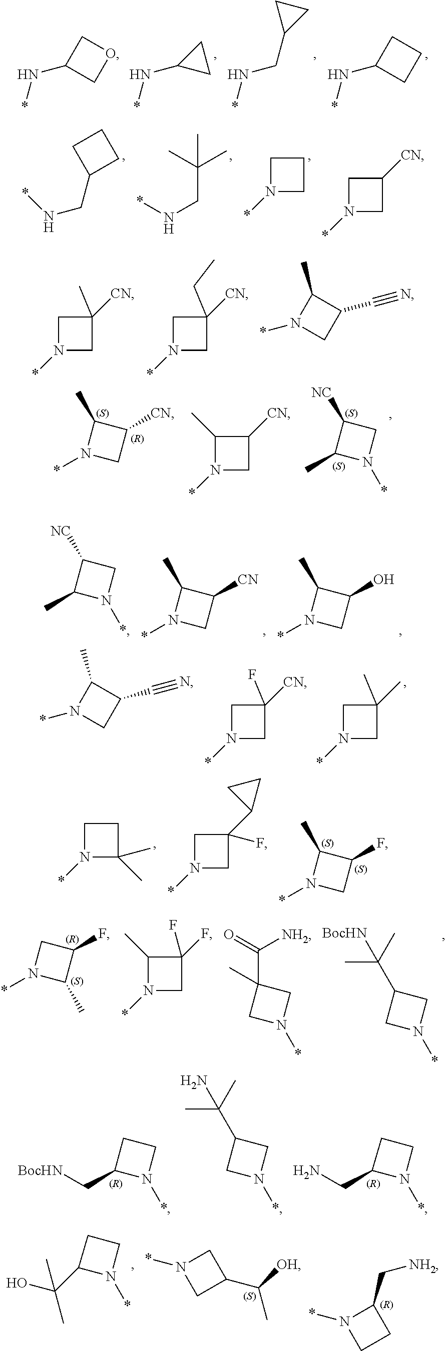

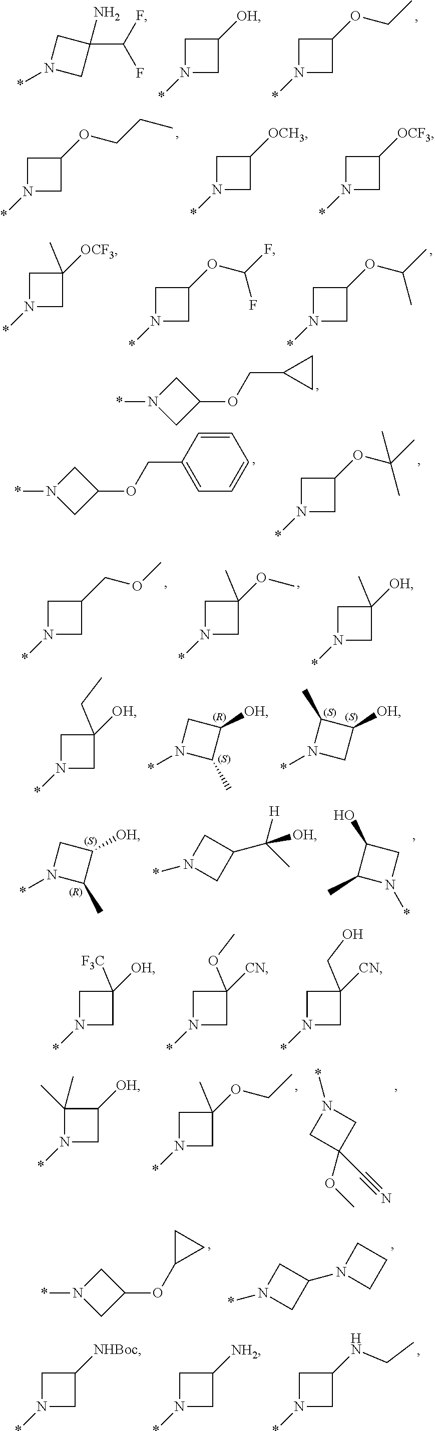

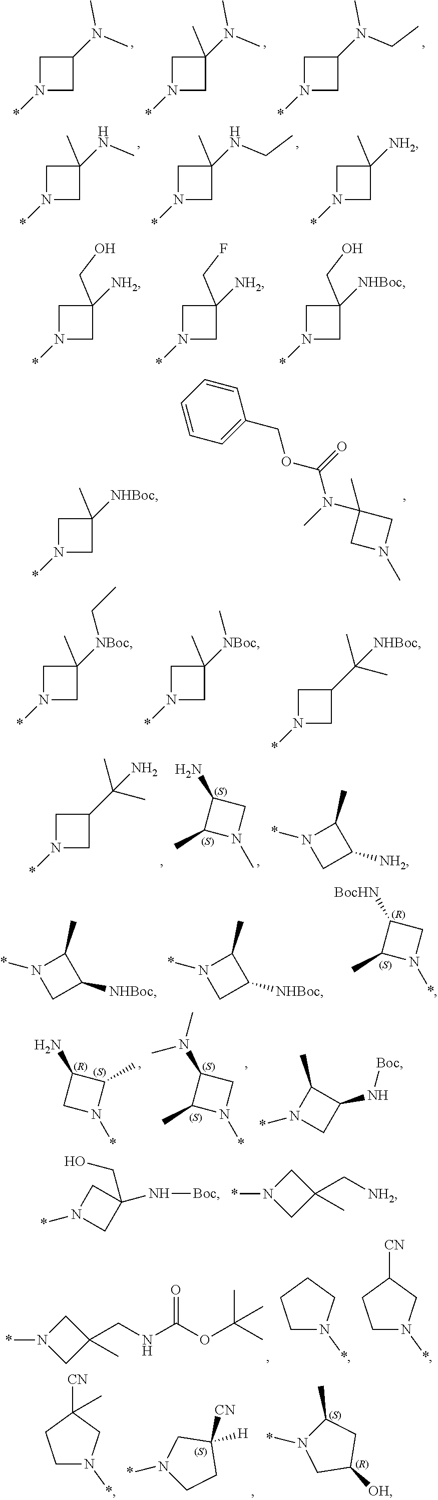

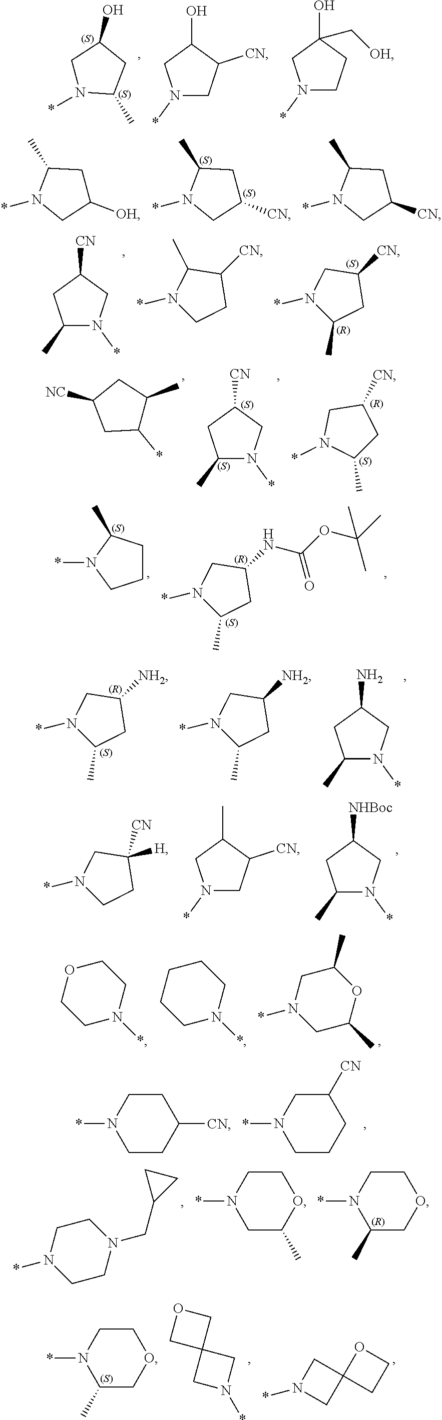

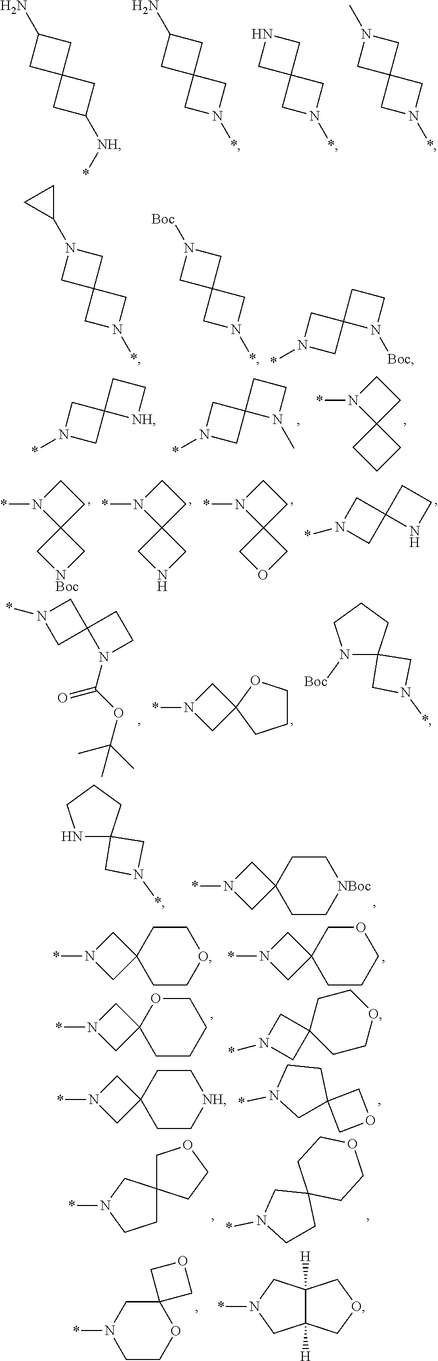

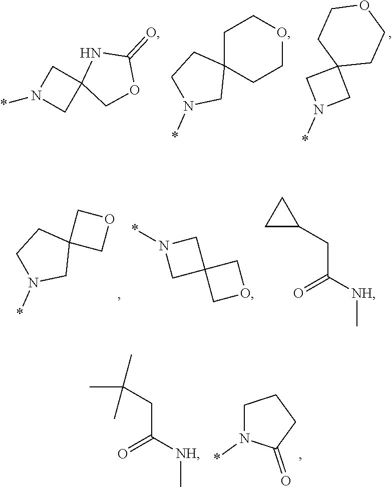

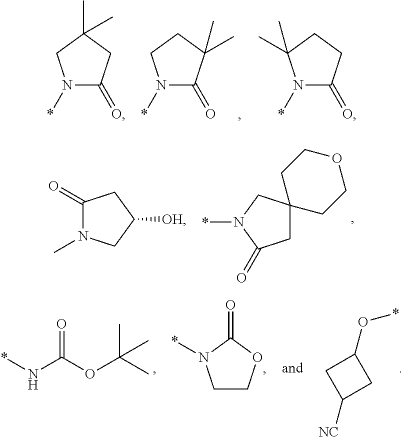

51. The compound, tautomer, solvate, stereoisomer, or pharmaceutically acceptable salt of claim 1, wherein R^{a}, for each occurrence, is independently selected from absent, CH₃, CF₂, F, Cl, CN, NH₂, NHCH₃, OH, —CH₂OH, —COOMe, —COOEt, —CONH₂, —C(═O)H, —CH₂CN, —CH₂NH₂, CF₃, NO₂,

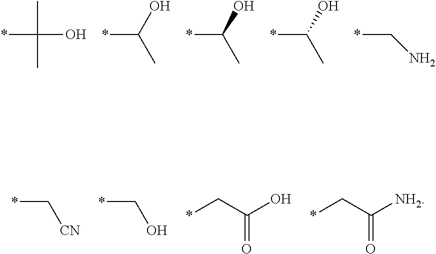

52. The compound, tautomer, solvate, stereoisomer, or pharmaceutically acceptable salt of claim 1, wherein R^{b}, for each occurrence, is independently selected from absent, halogen, ═O, and C₁-C₂ alkyl.

53. The compound, tautomer, solvate, stereoisomer, or pharmaceutically acceptable salt of claim 1, wherein R^{b}, for each occurrence, is independently selected from absent, —CH₃, ═O, F, and C₁.

54. The compound, tautomer, solvate, stereoisomer, or pharmaceutically acceptable salt of claim 1, wherein R^{c}, for each occurrence, is independently selected from absent, C₁-C₃ alkyl, CN, halogen, —OR^{s1}, and —C(═O)OR^{s1}, wherein R^{s1} is H or C₁-C₄ alkyl.

55. The compound, tautomer, solvate, stereoisomer, or pharmaceutically acceptable salt of claim 1, wherein R^{c}, for each occurrence, is independently selected from absent, CH₃, CN, F, Cl, —OCH₃, and —C(═O)OC(CH₃)₃.

56. The compound, tautomer, solvate, stereoisomer, or pharmaceutically acceptable salt of claim 1, wherein R₂ is selected from H, halogen, CN, OR^{s1}, —NR^{p1}R^{q1}, ═O, and C₁ to C₂ alkyl optionally substituted by 1 to 3 groups selected from halogen, wherein R^{s1}, R^{p1}, and R^{q1} are independently selected from H and CH₃.

57. The compound, tautomer, solvate, stereoisomer, or pharmaceutically acceptable salt of claim 1, wherein R₂ is selected from H, CH₃, CF₃, CN, F, Cl, Br, OH, OCH₃, NH₂, and ═O.

58. The compound, tautomer, solvate, stereoisomer, or pharmaceutically acceptable salt of claim 1, wherein R₁ and R₂ join to form a 5- to 6-membered ring optionally substituted by 1 to 2 groups selected from halogen and C₁ to C₂ alkyl optionally substituted by 1 to 2 groups selected from halogen.

59. The compound, tautomer, solvate, stereoisomer, or pharmaceutically acceptable salt of claim 1, wherein R₁ and R₂ join to form a 5- to 6-membered ring optionally substituted by 1 to 2 groups selected from F and CH₃.

60. The compound, tautomer, solvate, stereoisomer, or pharmaceutically acceptable salt of claim 1, wherein L is selected from —N(R^{x})—, —(CH₂)ᵤO(CH₂)ᵤ—, —(CH₂)ᵤS(═O)_{w}—(CH₂)ᵤ—, —S(═O)(═NR^{x})—, —(NR^{x})S(═O)_{w}—, —S(═O)_{w}(NR^{x})—, —C(═O)—, and C₁-C₃ alkylene, wherein the C₁-C₃ alkylene of L is optionally substituted by 1 to 2 groups selected from OH, C₁-C₃ alkyl, and ═CHR^{x}, wherein the C₁-C₃ alkyl of the C₁-C₃ alkylene of L optionally join to form a C₃-C₄ cycloalkyl, wherein R^{x} is selected from H and C₁-C₂ alkyl, u, for each occurrence, is independently 0 or 1.

61. The compound, tautomer, solvate, stereoisomer, or pharmaceutically acceptable salt of claim 1, wherein L is selected from

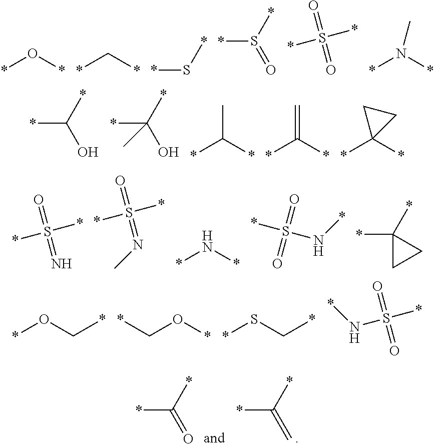

62. The compound, tautomer, solvate, stereoisomer, or pharmaceutically acceptable salt of claim 1, wherein L is selected from

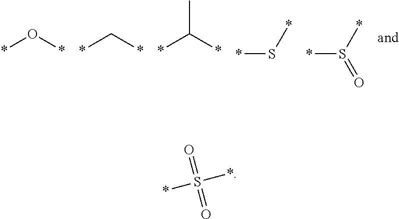

63. The compound according to claim 1, wherein the compound is selected from Compound 1 through Compound 702, a tautomer thereof, a solvate or stereoisomer of the compound or the tautomer, or a pharmaceutically acceptable salt of the foregoing.

64. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

65. A method of treating a disease or condition, comprising administering to a subject, a therapeutically effective amount of a compound according to claim 1, a tautomer thereof, a solvate or stereoisomer of the compound or the tautomer, or a pharmaceutically acceptable salt of the foregoing or the pharmaceutical composition comprising the compound, tautomer, solvate, stereoisomer, or pharmaceutically acceptable salt; wherein the disease or condition is selected from a inflammatory disease, an immune disease, an allergic disease, transplant rejection, a necrotic cell disease, a neurodegenerative disease, a central nervous system (CNS) disease, ischemic brain injury, an ocular disease, an infectious disease, and a malignancy.

66. The method according to claim 65, wherein the disease or condition is mediated by receptor-interacting protein 1 (RIP1) signaling.

67. A method of treating a disease or condition mediated by receptor-interacting protein 1 (RIP1) signaling, comprising administering to a subject, a therapeutically effective amount of a compound according to claim 1, a tautomer thereof, a solvate or stereoisomer of the compound or the tautomer, or a pharmaceutically acceptable salt of the foregoing or the pharmaceutical composition comprising the compound, tautomer, solvate, stereoisomer, or pharmaceutically acceptable salt.

68. The method according to claim 65, wherein the disease or condition is selected from ulcerative colitis, Crohn's disease, psoriasis, rheumatoid arthritis, amyotrophic lateral sclerosis (ALS), Alzheimer's disease, and a viral infection.

69. A method of inhibiting receptor-interacting protein 1 (RIP1), comprising contacting the RIP1 protein or a fragment thereof with a compound according to claim 1, a tautomer thereof, a solvate or stereoisomer of the compound or the tautomer, or a pharmaceutically acceptable salt of the foregoing or the pharmaceutical composition comprising the compound, tautomer, solvate, stereoisomer, or pharmaceutically acceptable salt.
